(19) United States
(12) Patent Application Publication
(10) Pub. No.: US 2023/0157155 A1
(43) Pub. Date: May 18, 2023

(54) LIGHT EMITTING DEVICE AND AMINE COMPOUND FOR LIGHT EMITTING DEVICE

(71) Applicant: Samsung Display Co., Ltd., Yongin-si (KR)

(72) Inventors: MINJI KIM, Hwaseong-si (KR); HANKYU PAK, Suwon-si (KR); DONGJUN KIM, Suwon-si (KR); Chaeyeong KIM, Yongin-si (KR); HYUNBIN PARK, Suwon-si (KR); BYEONGWOOK YOO, Hwaseong-si (KR); Jeongmin LEE, Hwaseong-si (KR); EUNJAE JEONG, Hwaseong-si (KR); SEOWON CHO, Asan-si (KR); SOHEE JO, Cheonan-si (KR); JIYONG CHOI, Cheonan-si (KR); SANGHYUN HAN, Hwaseong-si (KR)

(21) Appl. No.: 17/809,819

(22) Filed: Jun. 29, 2022

(30) Foreign Application Priority Data
Oct. 29, 2021 (KR) 10-2021-0146503
Jan. 5, 2022 (KR) 10-2022-0001805

(51) Int. Cl. H01L 51/00; C07C 211/61; C07F 7/08; C07D 209/88

(52) U.S. Cl. CPC H01L 51/006; C07C 211/61; H01L 51/0094; C07F 7/081; H01L 51/0061; C07D 209/88; H01L 51/0052; H01L 51/0058; H01L 51/0072; H01L 51/5064

(57) ABSTRACT

A light emitting device includes a first electrode, a second electrode on the first electrode, and at least one functional layer between the first electrode and the second electrode and including an amine compound represented by Formula 1.

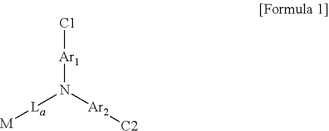

### CROSS-REFERENCE TO RELATED APPLICATIONS

This application claims priority to and the benefit of Korean Patent Application Nos. 10-2021-0146503, filed on Oct. 29, 2021, and 10-2022-0001805, filed on Jan. 5, 2022, the entire contents of each of which are hereby incorporated by reference.

### BACKGROUND

### 1. Field

Embodiments of the present disclosure herein relate to an amine compound used in a light emitting device, and, for example, to an amine compound used in a hole transport region, and a light emitting device including the same.

### 2. Related Art

Recently, the development of an organic electroluminescence display as an image display is being actively conducted. The organic electroluminescence display is different from a liquid crystal display and is a so-called a self-luminescent type display in which holes and electrons injected from a first electrode and a second electrode recombine in an emission layer so that a light emitting material including an organic compound in the emission layer emits light to achieve a display.

In the application of an organic electroluminescence device to a display, the decrease of a driving voltage and the increase of emission efficiency and life of the organic electroluminescence device are desired, and development of materials for an organic electroluminescence device stably achieving the requirements is being continuously conducted.

In addition, in order to achieve an organic electroluminescence device having high efficiency, development of a material for a hole transport region is being conducted.

### SUMMARY

Embodiments of the present disclosure provide a light emitting device having improved emission efficiency and device life.

Embodiments of the present disclosure also provide an amine compound which may improve the emission efficiency and device life of a light emitting device.

An embodiment of the present disclosure provides a light emitting device including a first electrode, a second electrode on the first electrode, and at least one functional layer between the first electrode and the second electrode, and including an amine compound represented by Formula 1 below.

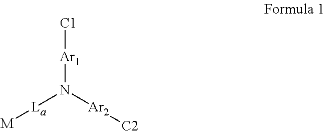

In Formula 1, Ar₁ and Ar₂ are each independently a substituted or unsubstituted arylene group of 6 to 30 ring-forming carbon atoms, or a substituted or unsubstituted heteroarylene group of 2 to 30 ring-forming carbon atoms, Lₐ is a direct linkage, a substituted or unsubstituted arylene group of 6 to 30 ring-forming carbon atoms, or a substituted or unsubstituted heteroarylene group of 2 to 30 ring-forming carbon atoms, C1 and C2 are each independently a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group, a case where both C1 and C2 are substituted or unsubstituted adamantyl groups is excluded, and M is represented by any one selected from among Formula 1-a to Formula 1-c below.

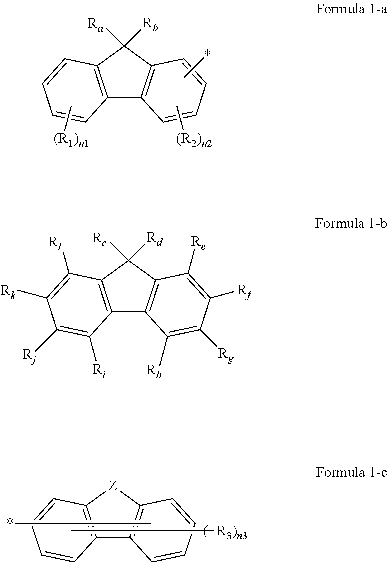

In Formula 1-a to Formula 1-c, Z is NR₄, O, or S, R₁ and R₂ are each independently a substituted or unsubstituted aryl group of 6 to 30 ring-forming carbon atoms, or a substituted or unsubstituted heteroaryl group of 2 to 30 ring-forming carbon atoms, and Rₐ to Rₗ, R₃, and R₄ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, in Formula 1-b, any one selected from among R_{c} to Rₗ is a part connected with Formula 1, in Formula 1-a and Formula 1-

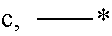 [SMILES: *C]

is a part connected with Formula 1, in a case where M in Formula 1 is represented by Formula 1-b or Formula 1-c, at least one selected from among C1 and C2 in Formula 1 is a bicycloheptanyl group, n1 is an integer of 0 to 4, n2 is an integer of 0 to 3, where the sum of n1 and n2 is 1 or more, and n3 is an integer of 0 to 7.

In an embodiment, the at least one functional layer may include an emission layer, a hole transport region between the first electrode and the emission layer, and an electron transport region between the emission layer and second electrode, and the hole transport region may include the amine compound.

In an embodiment, the amine compound represented by Formula 1 may be a monoamine compound.

In an embodiment, the amine compound represented by Formula 1 may be represented by any one selected from among Formula 2-1 to Formula 2-8 below.

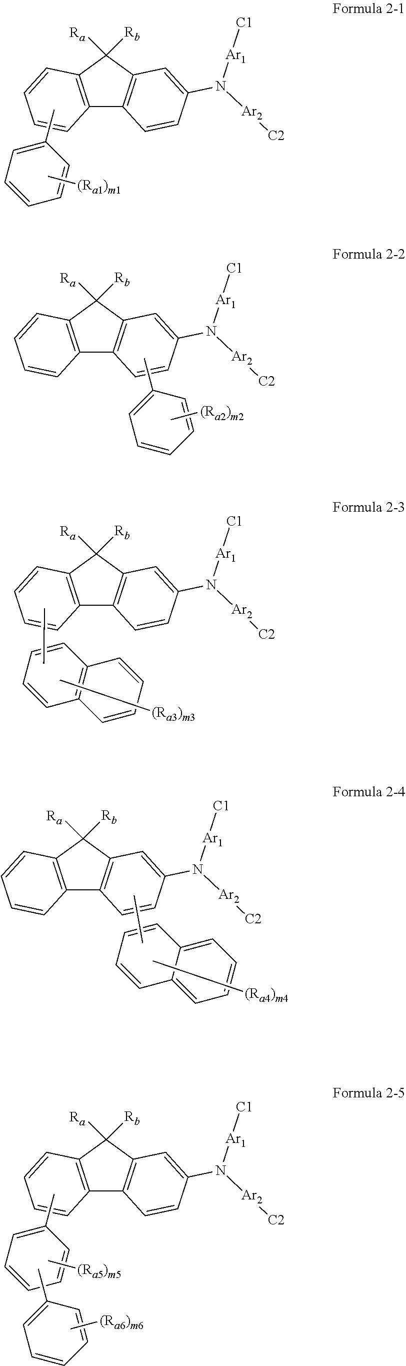

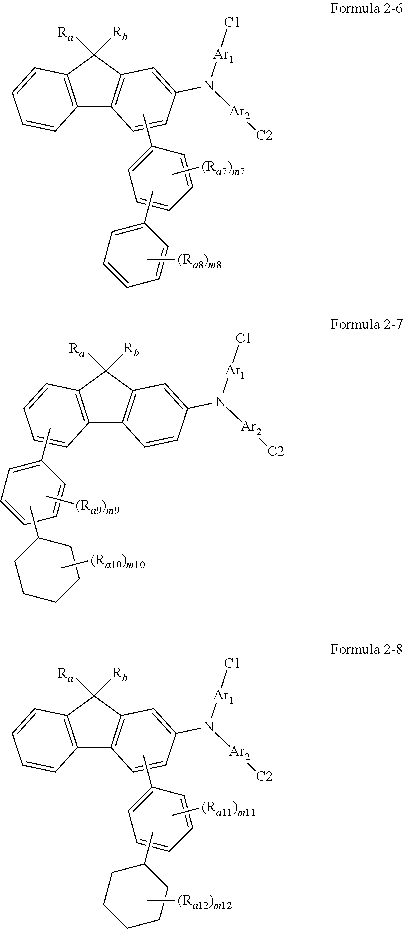

In Formula 2-1 to Formula 2-8, Rₐ₁ to Rₐ₁₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, m1, m2, m6, and m8 are each independently an integer of 0 to 5, m3 and m4 are each independently an integer of 0 to 7, m5, m7, m9, and m11 are each independently an integer of 0 to 4, and m10, and m12 are each independently an integer of 0 to 11.

In Formula 2-1 to Formula 2-8, the same description of Rₐ, R_{b}, Ar₁, Ar₂, C1 and C2 provided with respect to Formula 1 and Formula 1-a may be applied.

In an embodiment, the amine compound represented by Formula 1 may be represented by Formula 3-1 or Formula 3-2 below.

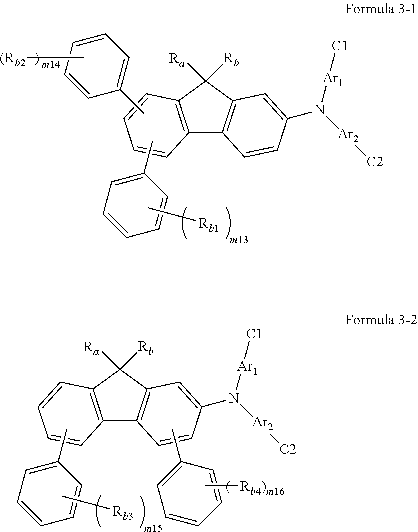

In Formula 3-1 and Formula 3-2, R_{b1} to R_{b4} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and m13 to m16 are each independently an integer of 0 to 5.

In Formula 3-1 and Formula 3-2, the same description of Rₐ, R_{b}, Ar₁, Ar₂, C1 and C2 provided with respect to Formula 1 and Formula 1-a may be applied.

In an embodiment, the amine compound represented by Formula 1 is represented by any one selected from among Formula 4-1 to Formula 4-15 below.

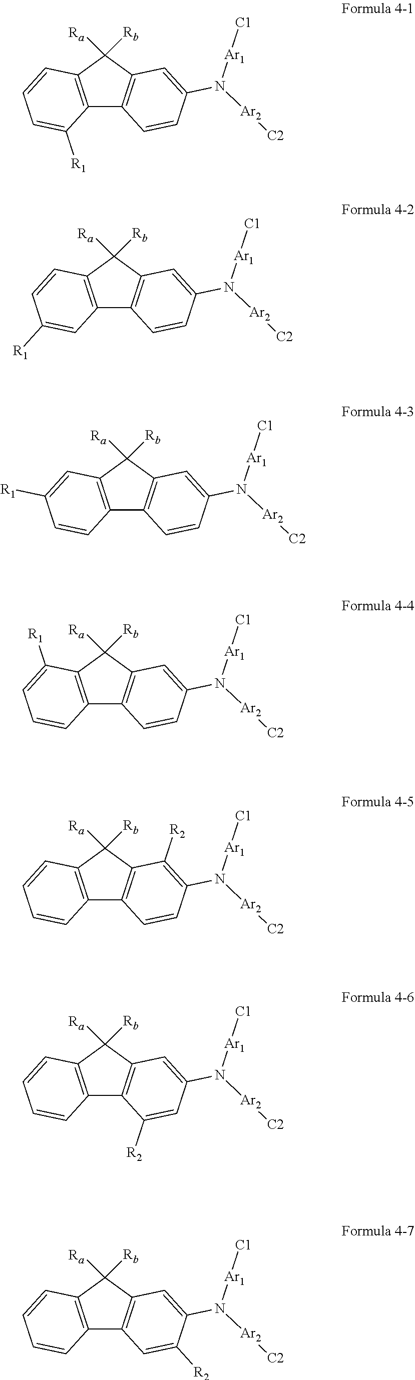

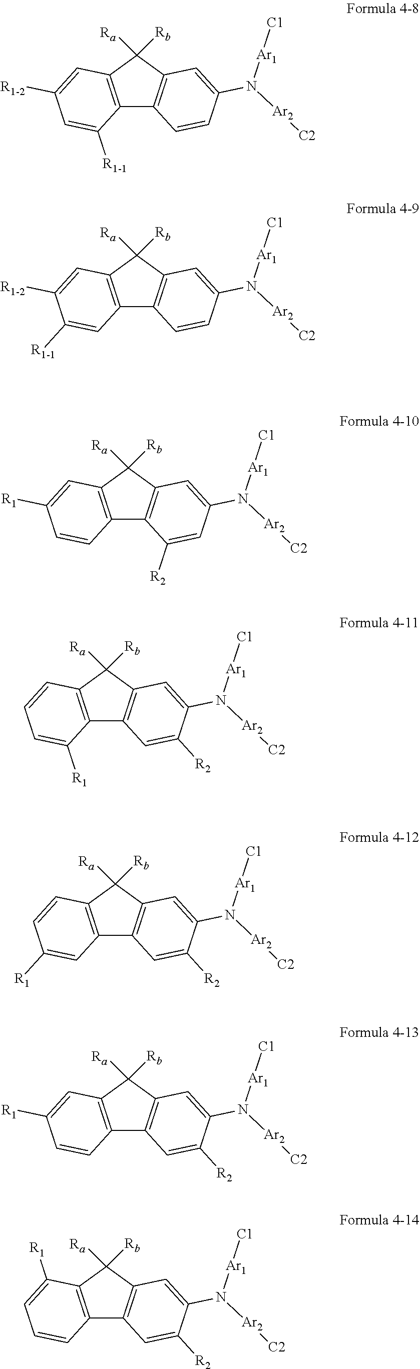

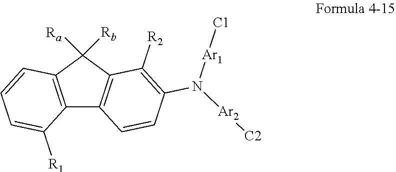

In Formula 4-1 to Formula 4-15, R₁₋₁ and R₁₋₂ may be each independently a substituted or unsubstituted aryl group of 6 to 30 ring-forming carbon atoms, or a substituted or unsubstituted heteroaryl group of 2 to 30 ring-forming carbon atoms.

In Formula 4-1 to Formula 4-15, the same description of R₁, R₂, Rₐ, R_{b}, Ar₁, Ar₂, C1, and C2 provided with respect to Formula 1 and Formula 1-a may be applied.

In an embodiment, the amine compound represented by Formula 1 may be represented by any one selected from among Formula 5-1 to Formula 5-3 below.

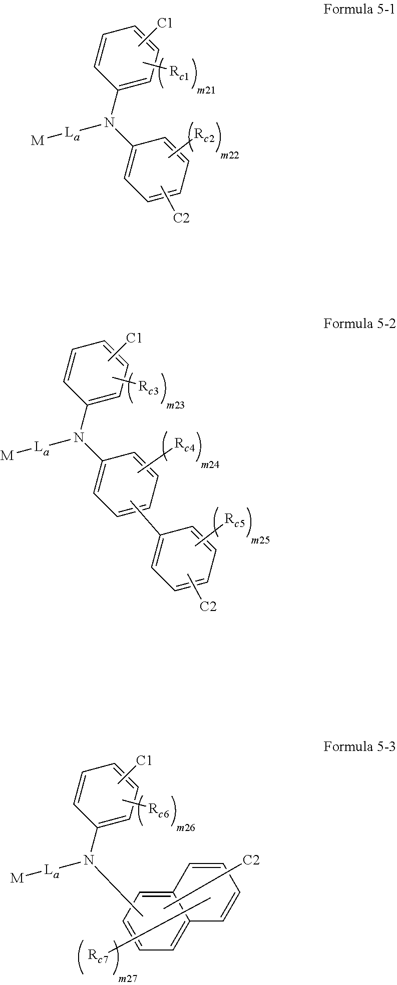

In Formula 5-1 to Formula 5-3, R_{c1} to R_{c7} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, m21 to m26 are each independently an integer of 0 to 4, and m27 is an integer of 0 to 6.

In Formula 5-1 to Formula 5-3, the same description of M, Lₐ, C1, and C2 provided with respect to Formula 1 may be applied.

In an embodiment, the amine compound represented by Formula 1 may be represented by any one selected from among Formula 6-1 to Formula 6-8 below.

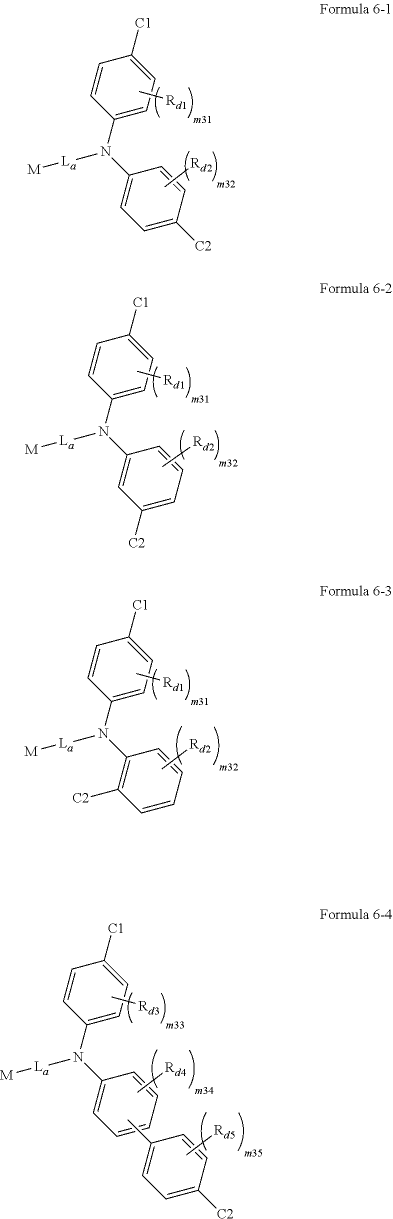

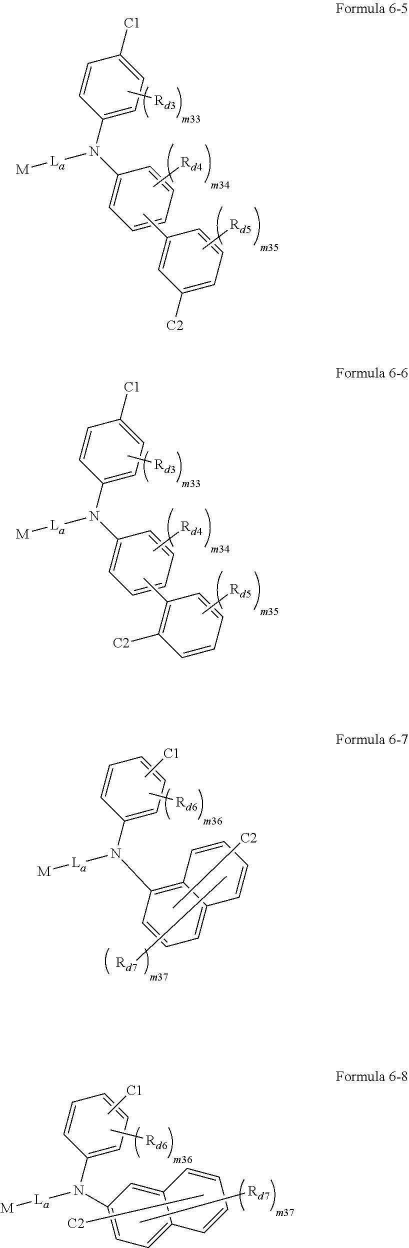

In Formula 6-1 to Formula 6-8, R_{d1} to R_{d7} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, m31 to m36 are each independently an integer of 0 to 4, and m37 is an integer of 0 to 6.

In Formula 6-1 to Formula 6-8, the same description of M, Lₐ, C1, and C2 provided with respect to Formula 1 may be applied.

In an embodiment, C1 and C2 may be each independently represented by any one selected from among Formula 7-1 to Formula 7-8 below.

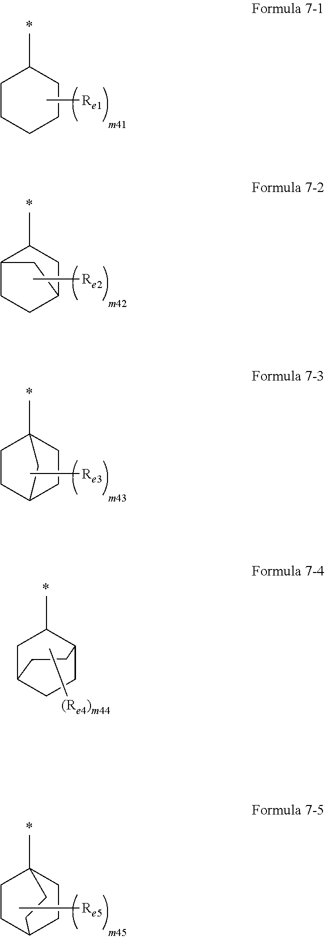

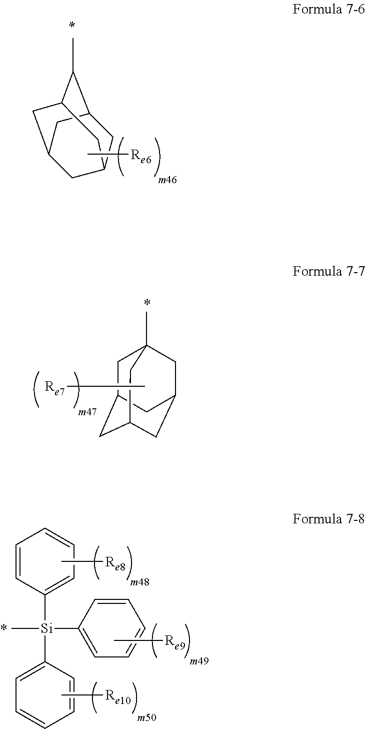

In Formula 7-1 to Formula 7-8, Rₑ₁ to Rₑ₁₀ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, m41 to m43 are each independently an integer of 0 to 11, m44 and m45 are each independently an integer of 0 to 13, m46 and m47 are each independently an integer of 0 to 15, and m48 to m50 are each independently an integer of 0 to 5.

In an embodiment, the amine compound represented by Formula 1 may be represented by any one selected from among Formula 8-1 to Formula 8-5 below.

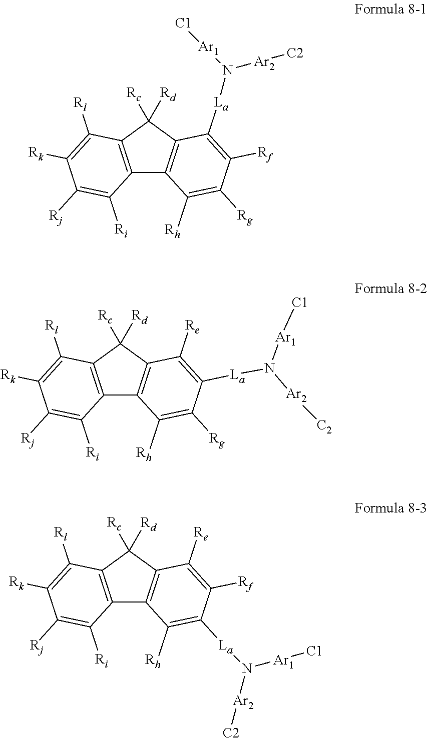

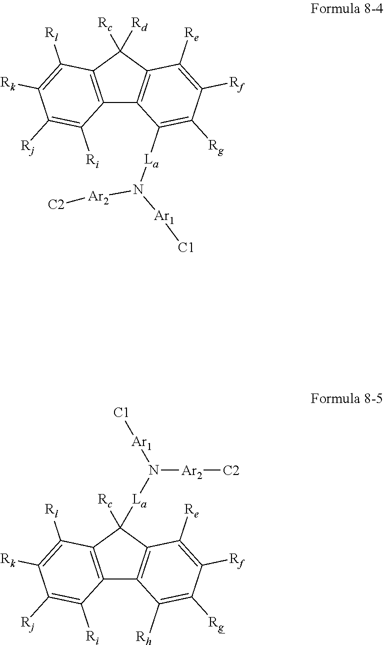

In Formula 8-1 to Formula 8-5, at least one selected from among C1 and C2 may be a substituted or unsubstituted bicycloheptanyl group.

In Formula 8-1 to Formula 8-5, the same description of Ar₁, Ar₂, Lₐ, C1, C2, and R_{c} to Rₗ provided with respect to Formula 1 and Formula 1-b may be applied.

In an embodiment, the amine compound represented by Formula 1 may be represented by any one selected from among Formula 9-1 to Formula 9-4 below.

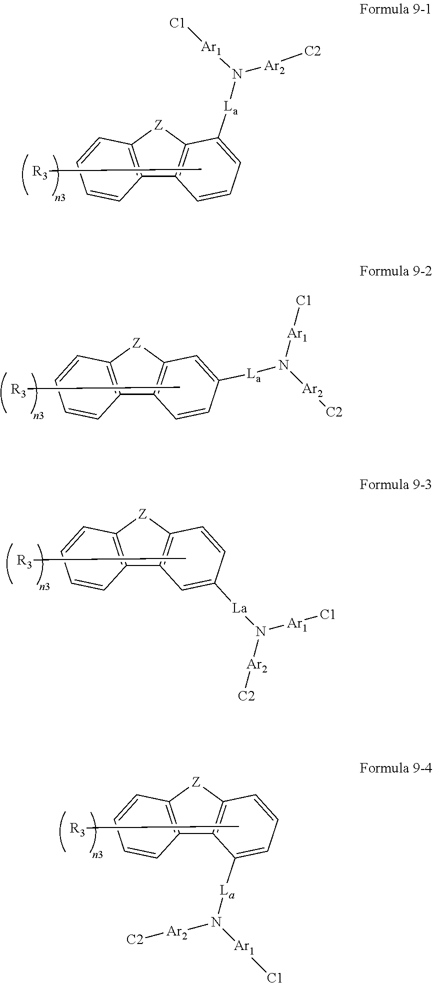

In Formula 9-1 to Formula 9-4, at least one selected from among C1 and C2 may be a substituted or unsubstituted bicycloheptanyl group.

In Formula 9-1 to Formula 9-4, the same description of Z, R₃, Ar₁, Ar₂, Lₐ, C1, and C2 provided with respect to Formula 1 and Formula 1-c may be applied.

In an embodiment, the amine compound may be at least one selected from among the compounds represented in Compound Group 1.

In an embodiment, the at least one functional layer may include an emission layer, a first hole transport layer between the first electrode and the emission layer, a second hole transport layer between the first hole transport layer and the emission layer, and an electron transport region between the emission layer and the second electrode, and the first hole transport layer may include the amine compound.

In an embodiment, the at least one functional layer may further include a third hole transport layer between the second hole transport layer and the emission layer, and the third hole transport layer may include the amine compound.

In an embodiment, the second hole transport layer may include an amine derivative compound represented by Formula 10 below.

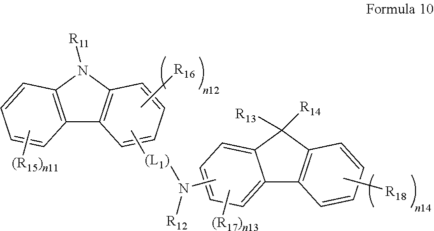

In Formula 10, L₁ is a direct linkage, a substituted or unsubstituted arylene group of 6 to 30 ring-forming carbon atoms, or a substituted or unsubstituted heteroarylene group of 2 to 30 ring-forming carbon atoms, R₁₁ to R₁₄ are each independently a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, R₁₅ to R₁₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, n11 and n14 are each independently an integer of 0 to 4, and n12 and n13 are each independently an integer of 0 to 3.

In an embodiment, the amine derivative compound represented by Formula 10 may be represented by Formula 11-1 or Formula 11-2 below.

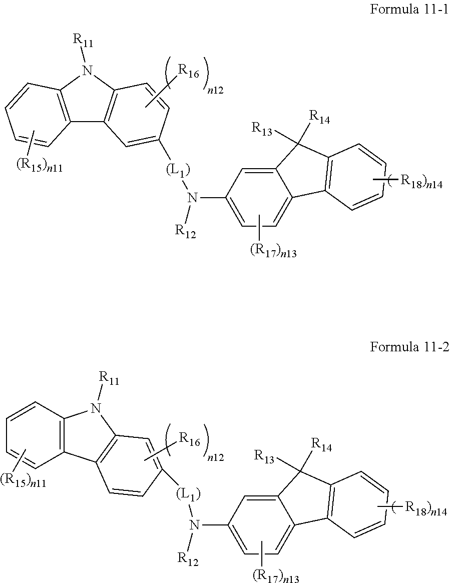

In Formula 11-1 and Formula 11-2, the same description of L₁, R₁₁ to R₁₄, R₁₅ to R₁₈, and n11 to n14 provided with respect to Formula 10 may be applied.

In an embodiment, the amine derivative compound represented by Formula 10 may be represented by any one selected from among compounds represented in Compound Group 2.

In an embodiment, the at least one functional layer may include an emission layer, a hole transport layer between the first electrode and the emission layer, and a hole transport auxiliary layer between the hole transport layer and the emission layer, and the hole transport auxiliary layer may include the amine compound.

In an embodiment, the hole transport auxiliary layer may include a first hole transport auxiliary layer on the hole transport layer, and a second hole transport auxiliary layer on the first hole transport auxiliary layer, and the first hole transport auxiliary layer may include the amine compound.

In an embodiment, a refractive index of the first hole transport auxiliary layer in a wavelength range of about 450 nm to about 700 nm may be about 1.55 to about 1.80, and a refractive index of the second hole transport auxiliary layer in a wavelength range of about 450 nm to about 700 nm may be about 1.65 to about 1.90.

In an embodiment, an absolute value of the highest occupied molecular orbital (HOMO) energy level of the second hole transport auxiliary layer may be greater than an absolute value of the HOMO energy level of the first hole transport auxiliary layer.

An amine compound according to an embodiment of the present disclosure is represented by Formula 1 above.

### BRIEF DESCRIPTION OF THE FIGURES

The accompanying drawings are included to provide a further understanding of the subject matter of the present disclosure and are incorporated in and constitute a part of this specification. The drawings illustrate embodiments of the present disclosure and, together with the description, serve to explain principles of the present disclosure. In the drawings:

FIG. 1 is a plan view of a display apparatus according to an embodiment of the present disclosure;

FIG. 2 is a cross-sectional view of a display apparatus according to an embodiment of the present disclosure;

FIG. 3 is a cross-sectional view schematically showing a light emitting device according to an embodiment of the present disclosure;

FIG. 4 is a cross-sectional view schematically showing a light emitting device according to an embodiment of the present disclosure;

FIG. 5 is a cross-sectional view schematically showing a light emitting device according to an embodiment of the present disclosure;

FIG. 6 is a cross-sectional view schematically showing a light emitting device according to an embodiment of the present disclosure;

FIG. 7 is a cross-sectional view schematically showing a light emitting device according to an embodiment of the present disclosure;

FIG. 8 is a cross-sectional view schematically showing a light emitting device according to an embodiment of the present disclosure;

FIG. 9 and FIG. 10 are respective cross-sectional views of display apparatuses according to embodiments;

FIG. 11 is a cross-sectional view showing a display apparatus according to an embodiment; and

FIG. 12 is a cross-sectional view showing a display apparatus according to an embodiment.

### DETAILED DESCRIPTION

The subject matter of the present disclosure may have various modifications and may be embodied in different forms, and example embodiments will be explained in more detail with reference to the accompany drawings. The subject matter of the present disclosure may, however, be embodied in different forms and should not be construed as limited to the embodiments set forth herein. Rather, the present disclosure is intended to cover all modifications, equivalents, and substituents which are included in the spirit and technical scope of the appended claims, and equivalents thereof.

Like reference numerals refer to like elements throughout. In the drawings, the dimensions of structures may be exaggerated for clarity of illustration. It will be understood that, although the terms first, second, etc. may be used herein to describe various elements, these elements should not be limited by these terms. These terms are only used to distinguish one element from another element. Thus, a first element could be termed a second element without departing from the spirit and scope of the present disclosure. Similarly, a second element could be termed a first element. As used herein, the singular forms are intended to include the plural forms as well, unless the context clearly indicates otherwise.

In the present description, it will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

In the present description, when a layer, a film, a region, a plate, etc. is referred to as being “on” or “above” another part, it can be “directly on” the other part, or intervening layers may also be present. On the contrary, when a layer, a film, a region, a plate, etc. is referred to as being “under” or “below” another part, it can be “directly under” the other part, or intervening layers may also be present. Also, when an element is referred to as being “on” another element, it can be under the other element.

In the present description, the term “substituted or unsubstituted” corresponds to substituted or unsubstituted with at least one substituent selected from the group consisting of a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the exemplified substituents may be substituted or unsubstituted. For example, a biphenyl group may be interpreted as an aryl group or a phenyl group substituted with a phenyl group.

In the present description, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the present description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the present description, the halogen atom may be a fluorine atom, a chlorine atom, a bromine atom or an iodine atom.

In the present description, the alkyl group may be a linear, branched or cyclic type. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the present description, a cycloalkyl group may be a cyclic alkyl group. The carbon number of the cycloalkyl group may be 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptanyl group, a bicyclooctanyl group, etc., without limitation.

In the present description, an aryl group may be an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the present description, a fluorenyl group may be substituted, and two substituents may be combined with each other to form a spiro structure. Examples of a substituted fluorenyl group are as follows, but an embodiment of the present disclosure is not limited thereto.

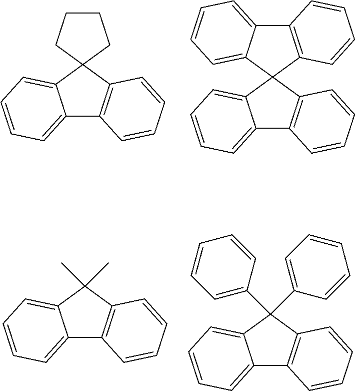

In the present description, a heteroaryl group may include one or more selected from among B, O, N, P, Si and S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the present description, the explanation of the aryl group may be applied to an arylene group except that the arylene group is a divalent group. The explanation on the heteroaryl group may be applied to a heteroarylene group except that the heteroarylene group is a divalent group.

In the present description, a silyl group may include an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the present description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

In the present description, a direct linkage may be a single bond.

In the present description,

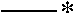 [SMILES: *C]

means a position to De connected.

Hereinafter, embodiments of the present disclosure will be further explained by referring to the drawings.

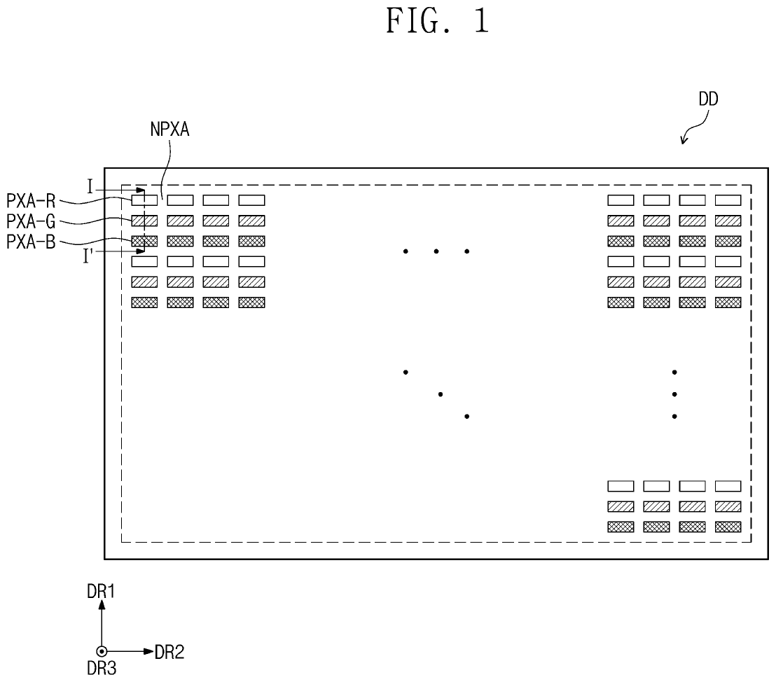

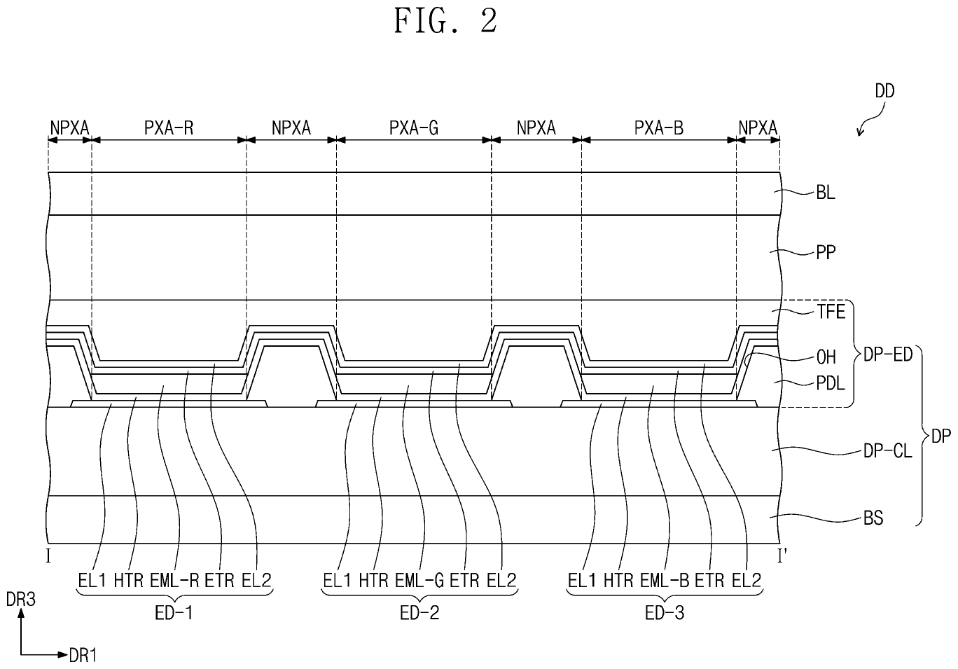

FIG. 1 is a plan view showing an embodiment of a display apparatus DD. FIG. 2 is a cross-sectional view of a display apparatus DD of an embodiment. FIG. 2 is a cross-sectional view showing a part corresponding to line I-I′ in FIG. 1.

The display apparatus DD may include a display panel DP and an optical layer PP on the display panel DP. The display panel DP includes light emitting devices ED-1, ED-2 and ED-3. The display apparatus DD may include multiple light emitting devices ED-1, ED-2 and ED-3. The optical layer PP may be on the display panel DP and control reflected light from external light at the display panel DP. The optical layer PP may include, for example, a polarization layer or a color filter layer. In some embodiments, different from the drawings, the optical layer PP may be omitted in the display apparatus DD of an embodiment.

A base substrate BL may be on the optical layer PP. The base substrate BL may be a member providing a base surface that the optical layer PP is on. The base substrate BL may be a glass substrate, a metal substrate, a plastic substrate, etc. However, an embodiment of the present disclosure is not limited thereto, and the base substrate BL may be an inorganic layer, an organic layer or a composite material layer. In addition, different from the drawings, the base substrate BL may be omitted in an embodiment.

The display apparatus DD according to an embodiment may further include a plugging layer. The plugging layer may be between a display device layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL provided on the base layer BS and a display device layer DP-ED. The display device layer DP-ED may include a pixel definition layer PDL, light emitting devices ED-1, ED-2 and ED-3 in the pixel definition layer PDL, and an encapsulating layer TFE on the light emitting devices ED-1, ED-2 and ED-3.

The base layer BS may be a member providing a base surface that the display device layer DP-ED is on. The base layer BS may be a glass substrate, a metal substrate, a plastic substrate, etc. However, an embodiment of the present disclosure is not limited thereto, and the base layer BS may be an inorganic layer, an organic layer or a composite material layer.

In an embodiment, the circuit layer DP-CL is on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting devices ED-1, ED-2 and ED-3 of the display device layer DP-ED.

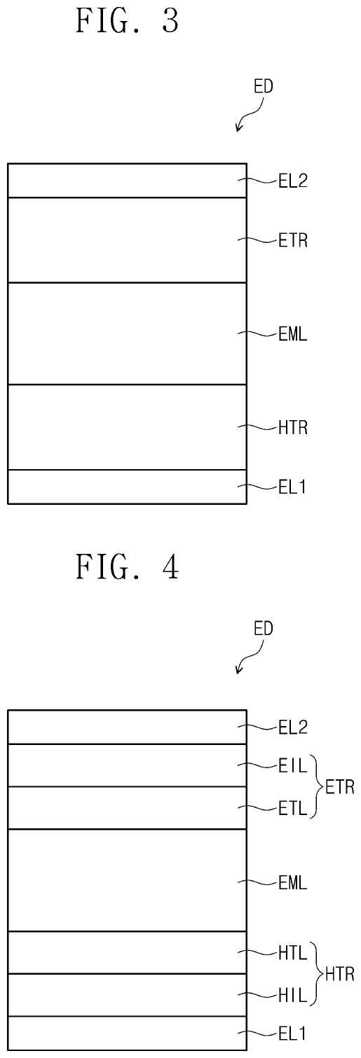

Each of the light emitting devices ED-1, ED-2 and ED-3 may have the structures of light emitting devices ED of embodiments according to FIG. 3 to FIG. 6, which will be further explained herein below. Each of the light emitting devices ED-1, ED-2 and ED-3 may include a first electrode EL1, a hole transport region HTR, emission layers EML-R, EML-G and EML-B, an electron transport region ETR, and a second electrode EL2.

FIG. 2 shows an embodiment where the emission layers EML-R, EML-G and EML-B of light emitting devices ED-1, ED-2 and ED-3 are in opening portions OH defined in a pixel definition layer PDL, and a hole transport region HTR, an electron transport region ETR and a second electrode EL2 are provided as common layers in all light emitting devices ED-1, ED-2 and ED-3. However, an embodiment of the present disclosure is not limited thereto. Different from FIG. 2, in an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G and EML-B, and the electron transport region ETR of the light emitting devices ED-1, ED-2 and ED-3 may be patterned by an ink jet printing method and provided.

An encapsulating layer TFE may cover the light emitting devices ED-1, ED-2 and ED-3. The encapsulating layer TFE may encapsulate the display device layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to an embodiment may include at least one inorganic layer (hereinafter, referred to as an encapsulating inorganic layer). In addition, the encapsulating layer TFE according to an embodiment may include at least one organic layer (hereinafter, encapsulating organic layer) and at least one encapsulating inorganic layer.

The encapsulating inorganic layer protects the display device layer DP-ED from moisture/oxygen, and the encapsulating organic layer protects the display device layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be on the second electrode EL2 and may be provided while filling the opening portion OH.

Referring to FIG. 1 and FIG. 2, the display apparatus DD may include a non-luminous area NPXA and luminous areas PXA-R, PXA-G and PXA-B. The luminous areas PXA-R, PXA-G and PXA-B may be areas that emit light produced from the light emitting devices ED-1, ED-2 and ED-3, respectively. The luminous areas PXA-R, PXA-G and PXA-B may be spaced apart from each other on a plane.

The luminous areas PXA-R, PXA-G and PXA-B may be areas spaced apart by the pixel definition layer PDL. The non-luminous areas NPXA may be areas between neighboring luminous areas PXA-R, PXA-G and PXA-B and may be areas corresponding to the pixel definition layer PDL. In the present disclosure, each of the luminous areas PXA-R, PXA-G and PXA-B may correspond to each pixel. The pixel definition layer PDL may divide the light emitting devices ED-1, ED-2 and ED-3. The emission layers EML-R, EML-G and EML-B of the light emitting devices ED-1, ED-2 and ED-3 may be respectively provided and divided in the opening portions OH defined in the pixel definition layer PDL.

The luminous areas PXA-R, PXA-G and PXA-B may be divided into multiple groups according to the color of light produced from the light emitting devices ED-1, ED-2 and ED-3. In the display apparatus DD of an embodiment, shown in FIG. 1 and FIG. 2, three luminous areas PXA-R, PXA-G and PXA-B that emit red light, green light and blue light, respectively, are illustrated as an embodiment. For example, the display apparatus DD of an embodiment may include a red luminous area PXA-R, a green luminous area PXA-G and a blue luminous area PXA-B, which are spaced apart from each other.

In the display apparatus DD according to an embodiment, multiple light emitting devices ED-1, ED-2 and ED-3 may emit light having different wavelength regions. For example, in an embodiment, the display apparatus DD may include a first light emitting device ED-1 emitting red light, a second light emitting device ED-2 emitting green light, and a third light emitting device ED-3 emitting blue light. In some embodiments, the red luminous area PXA-R, the green luminous area PXA-G, and the blue luminous area PXA-B of the display apparatus DD may correspond to the first light emitting device ED-1, the second light emitting device ED-2, and the third light emitting device ED-3, respectively.

However, an embodiment of the present disclosure is not limited thereto, and the first to third light emitting devices ED-1, ED-2 and ED-3 may emit light in the same wavelength region, or at least one thereof may emit light in a different wavelength region. For example, all the first to third light emitting devices ED-1, ED-2 and ED-3 may emit blue light.

The luminous areas PXA-R, PXA-G and PXA-B in the display apparatus DD according to an embodiment may be arranged in a stripe shape. Referring to FIG. 1, multiple red luminous areas PXA-R, multiple green luminous areas PXA-G and multiple blue luminous areas PXA-B may be arranged along a second directional axis DR2. In addition, the red luminous area PXA-R, the green luminous area PXA-G and the blue luminous area PXA-B may be arranged by turns along a first directional axis DR1.

In FIG. 1 and FIG. 2, the areas of the luminous areas PXA-R, PXA-G and PXA-B are shown similar, but an embodiment of the present disclosure is not limited thereto. The areas of the luminous areas PXA-R, PXA-G and PXA-B may be different from each other according to the wavelength region of light emitted. The areas of the luminous areas PXA-R, PXA-G and PXA-B may mean areas on a plane defined by the first directional axis DR1 and the second directional axis DR2.

The arrangement type of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various suitable combinations according to the properties of display quality required or desired for the display apparatus DD. For example, the arrangement type of the luminous areas PXA-R, PXA-G and PXA-B may be a PENTILE® arrangement structure (e.g., an RGBG matrix, RGBG structure, or RGBG matrix structure), or a diamond arrangement structure. PENTILE® is a duly registered trademark of Samsung Display Co., Ltd.

In addition, the areas of the luminous areas PXA-R, PXA-G and PXA-B may be different from each other. For example, in an embodiment, the area of the green luminous area PXA-G may be smaller than the area of the blue luminous area PXA-B, but an embodiment of the present disclosure is not limited thereto.

Hereinafter, FIG. 3 to FIG. 8 are cross-sectional views schematically showing light emitting devices according to embodiments. The light emitting device ED according to an embodiment may include a first electrode EL1, a second electrode EL2 facing the first electrode EL1, and at least one functional layer between the first electrode EL1 and the second electrode EL2. The at least one functional layer may include a hole transport region HTR, an emission layer EML, and an electron transport region ETR stacked in order. In some embodiments, the light emitting device ED of an embodiment may include a first electrode EL1, a hole transport region HTR, an emission layer EML, an electron transport region ETR, and a second electrode EL2 stacked in order.

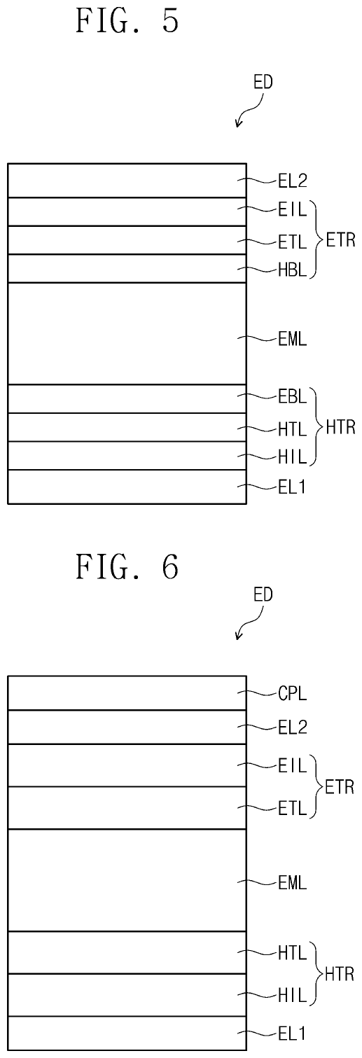

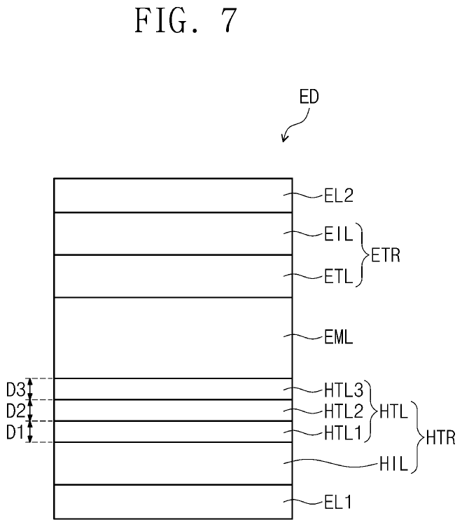

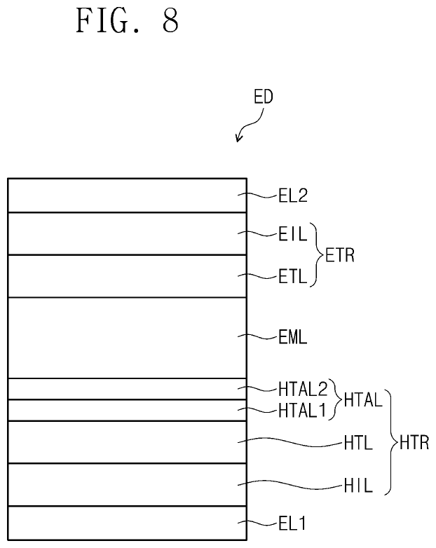

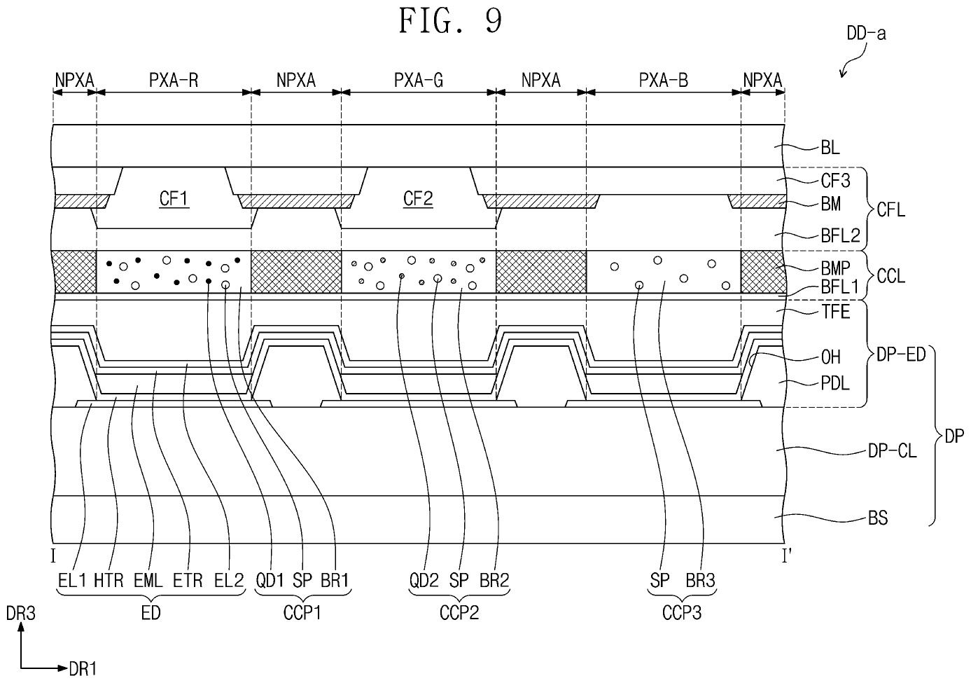

When compared with FIG. 3, FIG. 4 shows the cross-sectional view of a light emitting device ED of an embodiment, wherein a hole transport region HTR includes a hole injection layer HIL and a hole transport layer HTL, and an electron transport region ETR includes an electron injection layer EIL and an electron transport layer ETL. In addition, when compared with FIG. 3, FIG. 5 shows the cross-sectional view of a light emitting device ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 6 shows the cross-sectional view of a light emitting device ED of an embodiment, including a capping layer CPL on the second electrode EL2. When compared with FIG. 4, FIG. 7 shows the cross-sectional view of a light emitting device ED of an embodiment, including multiple hole transport layers HTL1, HTL2 and HTL3. When compared with FIG. 4, FIG. 8 shows the cross-sectional view of a light emitting device ED of an embodiment, wherein a hole transport region HTR includes a hole transport auxiliary layer HTAL between an emission layer EML and a hole transport layer HTL.

The light emitting device ED of an embodiment may include the amine compound of an embodiment, which will be further explained herein below, in at least one functional layer such as a hole transport region HTR, an emission layer EML, and an electron transport region ETR.

The first electrode EU has conductivity (e.g., electrical conductivity). The first electrode EL1 may be formed using a metal material, a metal alloy and/or a conductive compound. The first electrode EU may be an anode or a cathode. However, an embodiment of the present disclosure is not limited thereto. In addition, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, or oxides thereof.

If the first electrode EU is the transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is the transflective electrode or the reflective electrode, the first electrode EU may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds thereof, or mixtures thereof (for example, a mixture of Ag and Mg). Also, the first electrode EL1 may have a structure including multiple layers including a reflective layer or a transflective layer formed using the above materials, and a transmissive conductive layer formed using ITO, IZO, ZnO, or ITZO. For example, the first electrode EL1 may include a three-layer structure of ITO/Ag/ITO. However, an embodiment of the present disclosure is not limited thereto. The first electrode EL1 may include the above-described metal materials, combinations of two or more metal materials selected from the above-described metal materials, or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission auxiliary layer, or an electron blocking layer EBL.

The hole transport region HTR may have a single layer formed using a single material, a single layer formed using multiple different materials, or a multilayer structure including multiple layers formed using multiple different materials.

For example, the hole transport region HTR may have the structure of a single layer of a hole injection layer HIL or a hole transport layer HTL, and may have a structure of a single layer formed using a hole injection material and a hole transport material. In some embodiments, the hole transport region HTR may have a structure of a single layer formed using multiple different materials, or a structure stacked from the first electrode EL1 of hole injection layer HIL/hole transport layer HTL, hole injection layer HIL/hole transport layer HTL/buffer layer, hole injection layer HIL/buffer layer, hole transport layer HTL/buffer layer, or hole injection layer HIL/hole transport layer HTL/electron blocking layer EBL, without limitation.

If the hole transport region HTR includes the hole transport layer HTL, the hole transport layer HTL may have a single layer formed using a single material, a single layer formed using multiple different materials, or a multiple structure having multiple layers having multiple different materials. If the hole transport layer HTL has a multiple structure (e.g., a multilayer structure), as shown in FIG. 7, the hole transport layer HTL may include first to third hole transport layers HTL1, HTL2 and HTL3. The hole transport layer HTL may include a first hole transport layer HTL1 on the first electrode EL1, a second hole transport layer HTL2 on the first hole transport layer HTL1, and a third hole transport layer HTL3 on the second hole transport layer HTL2. In the light emitting device ED of an embodiment, the hole transport layer HTL may include multiple hole transport layers HTL1, HTL2 and HTL3 in the order of first hole transport layer HTL1/second hole transport layer HTL2/third hole transport layer HTL3 in a thickness direction.

Referring to FIG. 7, in the light emitting device ED of an embodiment, the hole transport region HTR may include a first hole transport layer HTL1 adjacent to a hole injection layer HIL, a third hole transport layer HTL3 adjacent to an emission layer EML, and a second hole transport layer HTL2 between the first hole transport layer HTL1 and the third hole transport layer HTL3. Each of the first hole transport layer HTL1 and the third hole transport layer HTL3 may include the amine compound of an embodiment, represented by Formula 1. The second hole transport layer HTL2 may include an amine derivative compound of an embodiment, represented by Formula 10.

In an embodiment, the first hole transport layer HTL1 and the third hole transport layer HTL3 may be layers having a refractive index smaller than that of the second hole transport layer HTL2. The first refractive index of the first hole transport layer HTL1 may be smaller than the second refractive index of the second hole transport layer HTL2, and the third refractive index of the third hole transport layer HTL3 may be smaller than the second refractive index of the second hole transport layer HTL2.

The hole transport region HTR may include the first to third hole transport layers HTL1, HTL2 and HTL3. Based on the second hole transport layer HTL2 having a relatively higher refractive index when compared to the first and third hole transport layers HTL1 and HTL3, the first hole transport layer HTL1 may be under the second hole transport layer HTL2, and the third hole transport layer HTL3 may be on the second hole transport layer HTL2. In the light emitting device ED of an embodiment, the hole transport region HTR may include multiple hole transport layers HTL1, HTL2 and HTL3 in the order of hole transport layer having a low refractive index/hole transport layer having a high refractive index/hole transport layer having a low refractive index in a thickness direction.

At a wavelength of about 460 nm, a difference between the first refractive index of the first hole transport layer HTL1 and the second refractive index of the second hole transport layer HTL2 may be greater than about 0.1. For example, at a wavelength of about 460 nm, a difference between the first refractive index and the second refractive index may be about 0.2 or more. In addition, at a wavelength of about 460 nm, a difference between the third refractive index of the third hole transport layer HTL3 and the second refractive index of the second hole transport layer HTL2 may be greater than about 0.1. For example, at a wavelength of about 460 nm, a difference between the third refractive index and the second refractive index may be about 0.2 or more.

At a wavelength of about 460 nm, the first refractive index of the first hole transport layer HTL1 and the third refractive index of the third hole transport layer HTL3 may be about 1.30 to about 1.80 for each. In addition, at a wavelength of about 460 nm, the second refractive index of the second hole transport layer HTL2 may be about 1.85 to about 2.40. For example, the first refractive index of the first hole transport layer HTL1 and the third refractive index of the third hole transport layer HTL3 may be about 1.40 to about 1.60 for each, and the second refractive index of the second hole transport layer HTL2 may be about 1.90 to about 2.00.

The thickness of the hole transport region HTR may be about 300 Å to about 15,000 Å, for example, the thickness of the hole transport region HTR may be about 300 Å to about 5,000 Å. The thicknesses D1, D2 and D3 of the first to third hole transport layers HTL1, HTL2 and HTL3, included in the hole transport region HTR may be about 100 Å to about 1,000 Å for each.

The thickness ratio (D1:D2:D3) of the first to third hole transport layers HTL1, HTL2 and HTL3, included in the hole transport region HTR may be about 0.1:0.8:0.1 to about 0.45:0.1:0.45. For example, in an embodiment, the thickness (D1) of the first hole transport layer HTL1 and the thickness (D3) of the third hole transport layer HTL3 may be substantially the same, and the thickness (D2) of the second hole transport layer HTL2 may be different from the thickness (D1) of the first hole transport layer HTL1 and the thickness (D3) of the third hole transport layer HTL3. However, an embodiment of the present disclosure is not limited thereto, and the thickness (D1) of the first hole transport layer HTL1 and the thickness (D3) of the third hole transport layer HTL3 may be different from each other. The thickness ratio (D1:D2:D3) of the first to third hole transport layers HTL1, HTL2 and HTL3 may be controlled to a suitable or optimal range according to the wavelength region of light emitted from the emission layer EML, the display quality required or desired for the display apparatus DD (FIG. 2), and the type of the hole transport materials used in each of the hole transport layers HTL1, HTL2 and HTL3 of the hole transport region HTR.

For example, in the light emitting device ED of an embodiment, if the emission layer EML emits blue light having a central wavelength in a wavelength region of about 430 nm to about 470 nm, the thickness ratio (D1:D2:D3) of the first to third hole transport layers HTL1, HTL2 and HTL3 may be 1:1:1. The light emitting device ED of an embodiment may include multiple hole transport layers HTL1, HTL2 and HTL3 in the order of hole transport layer having a low refractive index/hole transport layer having a high refractive index/hole transport layer having a low refractive index, and may show improved emission efficiency properties. The light emitting device ED of an embodiment includes the hole transport layers HTL1, HTL2 and HTL3 of the hole transport region HTR, having refractive index differences to minimize or reduce the destructive interference and extinction of light emitted from internal functional layers and to provide or improve constructive interference by the hole transport layers HTL1 HTL2 and HTL3, having refractive index differences, thereby showing high light extraction efficiency.

In some embodiments, the first hole transport layer HTL1 may be directly on the first electrode EL1. In addition, the third hole transport layer HTL3 may be directly under the emission layer EML.

In the present description, when a layer, a film, a region, a plate, etc. is referred to as being “directly on” the other part, it can mean that intervening layers, films, regions, plates, etc. are not present. For example, the “directly on” means that two layers are provided without using an additional member such as an adhesion member therebetween.

At a wavelength of about 460 nm in the light emitting device ED of an embodiment, the refractive index of the first electrode EL1 may be about 1.80 to about 2.40. For example, the refractive index of the first electrode EU may be about 1.90 to about 2.00. In some embodiments, the refractive index of the first electrode EU may be greater than the first refractive index of the first hole transport layer HTL1, and the refractive index difference at about 460 nm between adjacent first hole transport layer HTL1 and first electrode EL1 may be greater than about 0.1.

In addition, in the light emitting device ED of an embodiment, the refractive index of the emission layer EML at a wavelength of about 460 nm may be about 1.80 to about 2.24. For example, the refractive index of the emission layer EML may be about 1.90 to about 2.00. In some embodiments, the refractive index of the emission layer EML may be greater than the third refractive index of the third hole transport layer HTL3, and the refractive index difference at about 460 nm between adjacent third hole transport layer HTL3 and emission layer EML may be greater than about 0.1.

In some embodiments, the light emitting device ED of an embodiment includes a hole transport region HTR which includes hole transport layers HTL1 and HTL3, having refractive index differences from neighboring first electrode EL1 or emission layer EML, and may show high light extraction efficiency properties and improved emission efficiency properties.

The first hole transport layer HTL1 and the third hole transport layer HTL3 may each independently include an amine compound represented by Formula 1. The amine compound represented by Formula 1 may have a refractive index value of about 1.30 to about 1.80 at a wavelength of about 460 nm. Each of the first hole transport layer HTL1 and the third hole transport layer HTL3 may be formed using any one of the amine compounds represented by Formula 1, or mixtures thereof.

Referring to FIG. 8, the hole transport region HTR may further include a hole transport auxiliary layer HTAL between the emission layer EML and the hole transport layer HTL. The hole transport auxiliary layer HTAL may include a first hole transport auxiliary layer HTAL1 on the hole transport layer HTL, and a second hole transport auxiliary layer HTAL2 between the first hole transport auxiliary layer HTAL1 and the emission layer EML. The first hole transport auxiliary layer HTAL1 may include the amine compound of an embodiment, represented by Formula 1.

In an embodiment, the first hole transport auxiliary layer HTAL1 may be a layer having a smaller refractive index than the second hole transport auxiliary layer HTAL2. The fourth refractive index of the first hole transport auxiliary layer HTAL1 may be smaller than the fifth refractive index of the second hole transport auxiliary layer HTAL2.

At a wavelength of about 450 nm to about 700 nm, the difference between the fourth refractive index of the first hole transport auxiliary layer HTAL1 and the fifth refractive index of the second hole transport auxiliary layer HTAL2 may be greater than about 0.1. For example, at a wavelength of about 450 nm to about 700 nm, the difference between the fourth refractive index and the fifth refractive index may be about 0.2 or more. At a wavelength of about 450 nm to about 700 nm, the fourth refractive index of the first hole transport auxiliary layer HTAL1 may be about 1.55 to about 1.80. In addition, at a wavelength of about 450 nm to about 700 nm, the fifth refractive index of the second hole transport auxiliary layer HTAL2 may be about 1.65 to about 1.90.

In an embodiment, the first hole transport auxiliary layer HTAL1 may be directly on the hole transport layer HTL. In addition, the second hole transport auxiliary layer HTAL2 may be directly under the emission layer EML.

In an embodiment, the second hole transport auxiliary layer HTAL2 may have the highest occupied molecular orbital (HOMO) energy level which is deeper than that of the first hole transport auxiliary layer HTAL1.

In an embodiment, the HOMO energy levels of the hole transport layer HTL, the first hole transport auxiliary layer HTAL1, and the second hole transport auxiliary layer HTAL2 may satisfy Equation 1 below.

HOMO_{HTL}≥HOMO_{HTAL1}>HOMO_{HTAL2} Equation 1

In Equation 1, HOMO_{HTL} represents the HOMO energy level of the hole transport layer HTL, HOMO_{HTAL1} represents the HOMO energy level of the first hole transport auxiliary layer HTAL1, and HOMO_{HTAL2} represents the HOMO energy level of the second hole transport auxiliary layer HTAL2.

Referring to Equation 1, the HOMO energy level of the first hole transport auxiliary layer HTAL1 may be the same as the HOMO energy level of the hole transport layer HTL or deeper than the HOMO energy level of the hole transport layer. In addition, the HOMO energy level of the second hole transport auxiliary layer HTAL2 may be deeper than the HOMO energy level of the first hole transport auxiliary layer HTAL1. Because the first hole transport auxiliary layer HTAL1 which is adjacent to the hole transport layer HTL has the same as or deeper than the HOMO energy level of the hole transport layer HTL, holes may be easily injected. Because the second hole transport auxiliary layer HTAL2 has a deeper HOMO energy level than the first hole transport auxiliary layer HTAL1, the energy barrier of the emission layer EML becomes small, and holes may easily move to the emission layer EML.

In an embodiment, the HOMO energy level of the first hole transport auxiliary layer HTAL1 may be about −5.05 eV to about −5.30 eV. In addition, the HOMO energy level of the second hole transport auxiliary layer HTAL2 may be about −5.20 eV to about −5.40 eV. The light emitting device ED of an embodiment includes first and second hole transport auxiliary layers HTAL1 and HTAL2, having different energy levels, and hole transport capacity is improved, and excellent emission efficiency and improved device-life characteristics may be shown.

The first hole transport auxiliary layer HTAL1 may include the amine compound represented by Formula 1. The amine compound represented by Formula 1 may have the HOMO energy level of about −5.05 eV to about −5.30 eV. The first hole transport auxiliary layer HTAL1 may be formed using any one selected from among the amine compounds represented by Formula 1, or mixtures thereof.

In the light emitting device ED of an embodiment, the hole transport region HTR may include the amine compound of an embodiment. In the light emitting device ED of an embodiment, the hole transport layer HTL may include the amine compound represented by Formula 1. In addition, as shown in FIG. 7, if the light emitting device ED includes multiple hole transport layers HTL1, HTL2 and HTL3, the first hole transport layer HTL1 and/or the third hole transport layer HTL3 may include the amine compound represented by Formula 1.

The amine compound of an embodiment has a structure in which an amine group is connected with a first fused ring core and essentially includes a first substituent and a second substituent in a molecular structure. In an embodiment, the first fused ring may be a substituted or unsubstituted fluorenyl group, a substituted or unsubstituted dibenzofuran group, a substituted or unsubstituted dibenzothiophene group, or a substituted or unsubstituted carbazole group. In an embodiment, the first substituent and the second substituent may be each independently one substituent selected from among a substituted or unsubstituted adamantyl group, a substituted or unsubstituted cyclohexyl group, a substituted or unsubstituted cycloheptanyl group, a substituted or unsubstituted bicyclooctanyl group, and a substituted or unsubstituted triphenylsilyl group. In the present disclosure, the first fused ring may be any one substituent selected from among Formula 1-a to Formula 1-c, which will be further explained herein below.

The amine compound of an embodiment includes an amine group, and has a structure in which the nitrogen atom of the amine group is connected with one carbon atom selected from among the carbon atoms composing the first fused ring. The first fused ring and the nitrogen atom of the amine group may be directly linked, or combined via a first connecting group. The first substituent and the second substituent may be combined with the amine group of the amine compound of an embodiment via a second connecting group and a third connecting group, respectively. In an embodiment, the first connecting group to the third connecting group may be substituted or unsubstituted arylene groups of 6 to 30 ring-forming carbon atoms, or substituted or unsubstituted heteroarylene groups of 2 to 30 ring-forming carbon atoms. Because the amine compound of an embodiment, having such a structure shows high thermal properties, if applied to the light emitting device ED of an embodiment, the improvement of device life and efficiency may be achieved, holes and electrons in an emission layer EML may be balanced due to excellent charge transport capacity, the deposition temperature may be reduced during the deposition of the device, and thin film stability may be improved. In addition, the amine compound of an embodiment includes the first substituent and the second substituent, and the intermolecular interaction may be reduced, and low packing density may be achieved. Accordingly, the amine compound of an embodiment may have a low refractive index, and through diversely changing the combination of the first substituent and the second substituent, the refractive index of the molecule may be changed.

The amine compound of an embodiment may include at least one third substituent connected with the first fused ring, or may include a structure in which at least one selected from among the first substituent and the second substituent, which is connected with the nitrogen atom of the amine group, is a substituted or unsubstituted bicycloheptanyl group.

The amine compound of an embodiment may include at least one third substituent connected with the first fused ring. The third substituent may be a group connected with at least one benzene ring selected from among the benzene rings composing the first fused ring to expand a π-conjugation structure. For example, the third substituent may be a substituent having at least one or more π bonds which may be connected with the first fused ring and conjugated. In an embodiment, the third substituent may be a substituted or unsubstituted aryl group of 6 to 30 ring-forming carbon atoms, or a substituted or unsubstituted heteroaryl group of 2 to 30 ring-forming carbon atoms. By introducing at least one third substituent to the first fused ring, the π-conjugation may be expanded widely, the molecular stability of a polaron state may be improved, and accordingly, the device life and efficiency may be improved. In addition, the total molecular weight of the amine compound may be increased due to the third substituent, and there are benefits of markedly increasing the glass transition temperature. In the present disclosure, the third substituent may be the substituent represented by R₁ and R₂ in Formula 1-a.

Otherwise, the amine compound of an embodiment may include a structure in which at least one selected from among the first substituent and the second substituent connected with the nitrogen atom of the amine group is a substituted or unsubstituted bicycloheptanyl group. For example, the amine compound of an embodiment may include a first substituent which is one substituent selected from a substituted or unsubstituted adamantyl group, a substituted or unsubstituted cyclohexyl group, a substituted or unsubstituted bicycloheptanyl group, a substituted or unsubstituted bicyclooctanyl group, and a substituted or unsubstituted triphenylsilyl group, and a second substituent of a substituted or unsubstituted bicycloheptanyl group, connected with the nitrogen atom. In the amine compound of an embodiment, because at least one selected from among the first substituent and the second substituent includes the substituted or unsubstituted bicycloheptanyl group, the amine compound according to an embodiment, represented by Formula 1 may have high glass transition temperature properties, and due to such high glass transition temperature properties, excellent properties of heat resistance and durability may be shown.

The amine compound of an embodiment is represented by Formula 1 below.

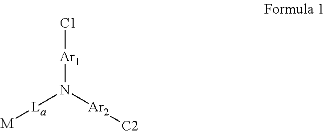

In Formula 1, Ar₁ and Ar₂ are each independently a substituted or unsubstituted arylene group of 6 to 30 ring-forming carbon atoms, or a substituted or unsubstituted heteroarylene group of 2 to 30 ring-forming carbon atoms. In an embodiment, Ar₁ and Ar₂ may be each independently a substituted or unsubstituted phenylene group, a substituted or unsubstituted divalent biphenyl group, or a substituted or unsubstituted divalent naphthyl group. For example, Ar₁ and Ar₂ may be each independently an unsubstituted phenylene group, an unsubstituted divalent biphenyl group, or an unsubstituted divalent naphthyl group.

In Formula 1, Lₐ is a direct linkage, a substituted or unsubstituted arylene group of 6 to 30 ring-forming carbon atoms, or a substituted or unsubstituted heteroarylene group of 2 to 30 ring-forming carbon atoms. In an embodiment, Lₐ may be a direct linkage, or a substituted or unsubstituted arylene group of 6 to 30 ring-forming carbon atoms. For example, Lₐ may be a direct linkage, or a substituted or unsubstituted phenylene group.

In Formula 1, C1 and C2 are each independently a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group. In some embodiments, the amine compound of an embodiment may include one first substituent selected from a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group combined with Ar₁, and one second substituent selected from a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group combined with Ar₂. For example, C1 and C2 may be each independently a substituted or unsubstituted adamantyl group, a substituted or unsubstituted cyclohexyl group, a substituted or unsubstituted bicyclo[2,2,1]heptanyl group, a substituted or unsubstituted bicyclo[2,2,2]octanyl group, or a substituted or unsubstituted triphenylsilyl group. In an embodiment, C1 and C2 may be the same or different. However, a case where both C1 and C2 are substituted or unsubstituted adamantyl groups is excluded from Formula 1.

In Formula 1, M is represented by any one selected from among Formula 1-a to Formula 1-c below. The amine compound represented by Formula 1 may include a first fused ring which is any one selected from Formula 1-a to Formula 1-c below. The amine group in the amine compound represented by Formula 1 may be connected with the first fused ring which is any one selected from Formula 1-a to Formula 1-c below.

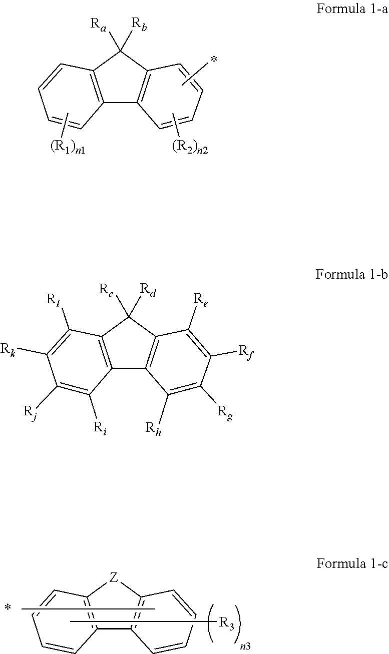

In Formula 1-c, Z is NR₄, O, or S.

In Formula 1-a, R₁ and R₂ are each independently a substituted or unsubstituted aryl group of 6 to 30 ring-forming carbon atoms, or a substituted or unsubstituted heteroaryl group of 2 to 30 ring-forming carbon atoms. In an embodiment, R₁ and R₂ may be each independently a substituted or unsubstituted aryl group of 6 to 30 ring-forming carbon atoms. For example, R₁ and R₂ may be each independently a substituted or unsubstituted phenyl group, a substituted or unsubstituted biphenyl group, or a substituted or unsubstituted naphthyl group. R₁ and R₂ may be each independently an unsubstituted phenyl group, an unsubstituted biphenyl group, or an unsubstituted naphthyl group.

In Formula 1-b, Rₐ to Rₗ, R₃ and R₄ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. Otherwise, each of Rₐ to Rₗ, R₃ and R₄ is combined with an adjacent group to form a ring. In an embodiment, Rₐ to R_{d} may be each independently a substituted or unsubstituted alkyl group of 1 to 10 carbon atoms, or a substituted or unsubstituted aryl group of 6 to 20 ring-forming carbon atoms. In addition, each group of Rₐ and R_{b}, and R_{c} and R_{d} may be combined with each other to form a ring. If Rₐ and R_{b} are combined with each other to form a ring, the substituent represented by Formula 1-a may have a spiro structure. In addition, if R_{c} and R_{c} are combined with each other to form a ring, the substituent represented by Formula 1-b may have a spiro structure. In an embodiment, Rₐ to Rₗ, R₃ and R₄ may be each independently a hydrogen atom.

In Formula 1-b, any one selected from among R_{c} to Rₗ is a part connected with Formula 1. In some embodiments, in Formula 1-b, in R_{c} to Rₗ, the remaining substituents excluding the substituent combined with the nitrogen atom of the amine compound represented by Formula 1 may be each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring. For example, in R_{c} to Rₗ, the remaining substituents excluding the substituent combined with the nitrogen atom of the amine compound represented by Formula 1 may be a hydrogen atom.

In Formula 1-a and Formula 1-c,

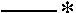 [SMILES: *C]

is a part connected with Formula 1.

In Formula 1-a, n1 is the number of R₁, and n1 is an integer of 0 to 4. n2 is the number of R₂ and is an integer of 0 to 3.

If n1 is 0, the amine compound of an embodiment may not be substituted with R₁. Formula 1-a where n1 is 4, and all of R₁ are hydrogen atoms, may be the same as Formula 1-a where n1 is 0. If n1 is an integer of 2 or more, multiple R₁ may all be the same, or at least one selected from among multiple R₁ may be different.

If n2 is 0, the amine compound of an embodiment may not be substituted with R₂. Formula 1-a where n2 is 3, and all of R₂ are hydrogen atoms, may be the same as Formula 1-a where n2 is 0. If n2 is an integer of 2 or more, multiple R₂ may all be the same, or at least one selected from among multiple R₂ may be different.

In Formula 1-a, the sum of n1 and n2 is 1 or more. If M is represented by Formula 1-a in Formula 1, the first fused ring represented by Formula 1-a in the amine compound of an embodiment may include at least one third substituent. In the amine compound of an embodiment, the first fused ring represented by Formula 1-a may include at least one substituent represented by R₁, or at least one substituent represented by R₂. In an embodiment, n1 and n2 may be each independently an integer of 0 to 2, and the sum of n1 and n2 may be 1 or 2. For example, if the sum of n1 and n2 is 1, n1 may be 1, and n2 may be 0 in Formula 1, or n1 may be 0, and n2 may be 1 in Formula 1. If the sum of n1 and n2 is 2, n1 and n2 of Formula 1 may be 1 each, or n1 may be 2, and n2 may be 0. However, an embodiment of the present disclosure is not limited thereto.

In Formula 1, if M is represented by Formula 1-b or Formula 1-c, at least one selected from among C1 and C2 in Formula 1 may be a substituted or unsubstituted bicycloheptanyl group. In some embodiments, if M is represented by Formula 1-b or Formula 1-c in Formula 1, the amine compound represented by Formula 1 may essentially include a substituted or unsubstituted bicycloheptanyl group. For example, the amine compound represented by Formula 1 may include one first substituent selected from a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group, combined with Ar₁, and a second substituent of a substituted or unsubstituted bicycloheptanyl group combined with Ar₂.

In Formula 1-c, n3 is an integer of 0 to 7. If n3 is 0, the amine compound of an embodiment may not be substituted with R₃. Formula 1-c where n3 is 7, and all of R₃ are hydrogen atoms, may be the same as Formula 1-c where n3 is 0. If n3 is an integer of 2 or more, multiple R₃ may all be the same, or at least one selected from among multiple R₃ may be different.

In an embodiment, the amine compound represented by Formula 1 may be represented by Formula 1-1 below.

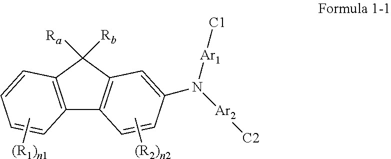

Formula 1-1 represents a case of the structure of Formula 1 where M is represented by Formula 1-a, and the carbon position of the first fused ring represented by Formula 1-a, to which the nitrogen atom of Formula 1 is bonded is specified. The amine compound of an embodiment, represented by Formula 1 is directly bonded to a fluorenyl group represented by Formula 1-a, and in this case, the nitrogen atom of the amine group may be connected with the carbon at position 2 or carbon at position 7 of the fluorenyl group represented by Formula 1-a. In some embodiments, the carbon numbers of the fluorenyl group are shown in Formula a shown below.

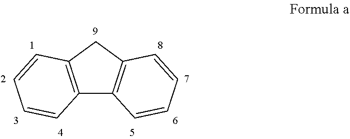 [SMILES: c1ccc2c(c1)Cc1ccccc1-2]

In Formula 1-1, the same description of Rₐ, R_{b}, R₁, R₂, n1, n2, Ar₁, Ar₂, C1 and C2 provided with respect to Formula 1 and Formula 1-a may be applied.

In an embodiment, the amine compound represented by Formula 1 may be represented by any one selected from among Formula 2-1 to Formula 2-8 below.

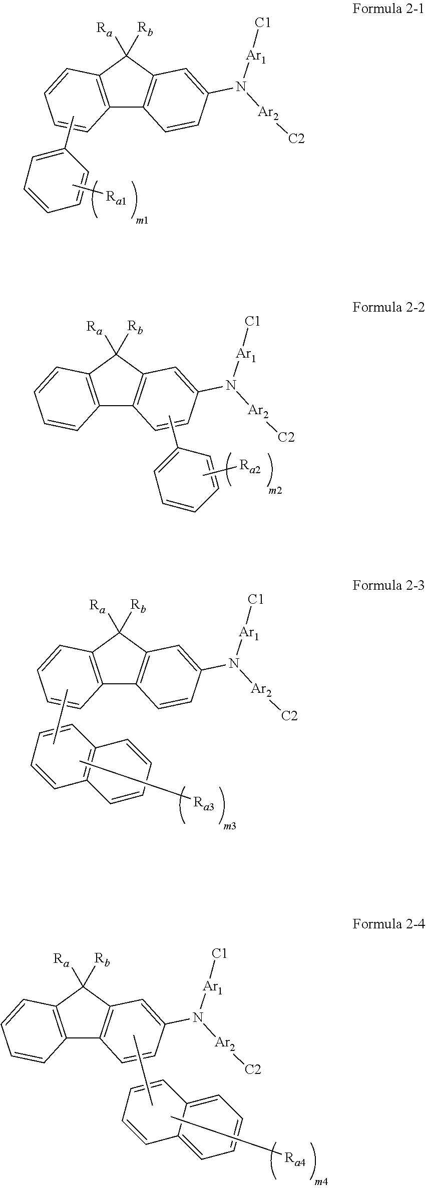

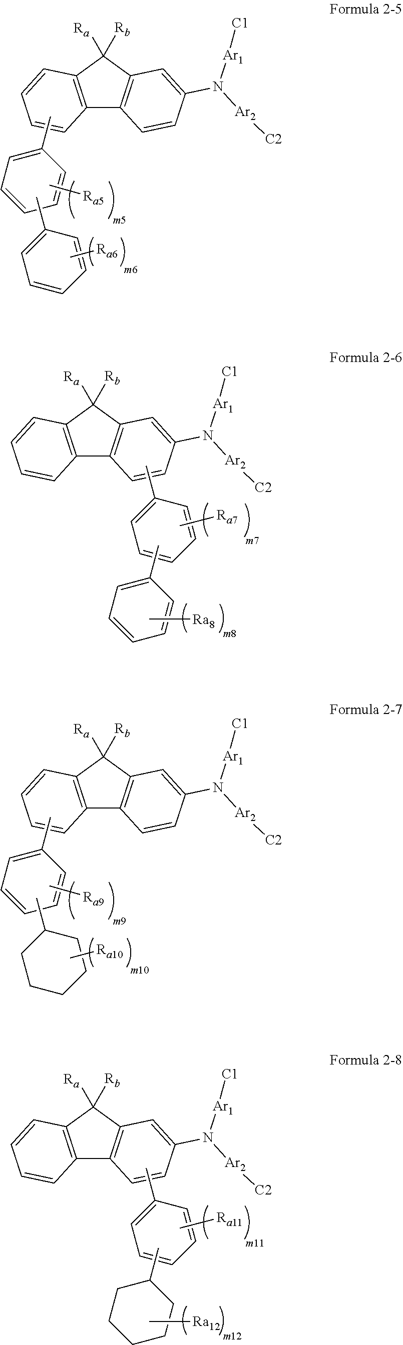

Formula 2-1 to Formula 2-8 represent cases of Formula 1 where M is represented by Formula 1-a, the carbon position of the first fused ring structure represented by Formula 1-a to which the nitrogen atom of Formula 1 is bonded is specified, and the number and type of substituents connected with the substituent represented by Formula 1-a are specified. For example, in the structure of Formula 1-a, the number and type of R₁ and R₂ are specified.

Formula 2-1 represents a case of Formula 1 where M is represented by Formula 1-a, n1 is 1, and n2 is 0 in Formula 1-a, and the substituent represented by R₁ is a substituted or unsubstituted phenyl group. Formula 2-2 represents a case where n1 is 0, and n2 is 1 in Formula 1-a, and the substituent represented by R₂ is a substituted or unsubstituted phenyl group. Formula 2-3 represents a case of Formula 1 where n1 is 1, and n2 is 0 in Formula 1-a, and the substituent represented by R₁ is a substituted or unsubstituted naphthyl group. Formula 2-4 represents a case where n1 is 0, and n2 is 1 in Formula 1-a, and the substituent represented by R₂ is a substituted or unsubstituted naphthyl group. Formula 2-5 represents a case where n1 is 1, and n2 is 0 in Formula 1-a, and the substituent represented by R₁ is a substituted or unsubstituted biphenyl group. Formula 2-6 represents a case where n1 is 0, and n2 is 1 in Formula 1-a, and the substituent represented by R₂ is a substituted or unsubstituted biphenyl group. Formula 2-7 represents a case where n1 is 1, and n2 is 0 in Formula 1-a, and the substituent represented by R₁ is a substituted or unsubstituted cyclohexylphenyl group. Formula 2-8 represents a case where n1 is 0, and n2 is 1 in Formula 1-a, and the substituent represented by R₂ is a substituted or unsubstituted cyclohexylphenyl group.

In Formula 2-1 to Formula 2-8, Rₐ₁ to Rₐ₁₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Rₐ₁ to Rₐ₁₂ may be each independently a hydrogen atom, or a substituted or unsubstituted phenyl group.

In Formula 2-1 to Formula 2-8, m1, m2, m6, and m8 are each independently an integer of 0 to 5, m3 and m4 are each independently an integer of 0 to 7, m5, m7, m9, and m11 are each independently an integer of 0 to 4, and m10 and m12 are each independently an integer of 0 to 11.

In the cases where m1, m2, m6, and m8 are 0, the amine compounds of embodiments may be unsubstituted with Rₐ₁, Rₐ₂, Rₐ₆, and Rₐ₈, respectively. The cases where m1, m2, m6, and m8 are 5, and Rₐ₁, Rₐ₂, Rₐ₆, and Rₐ₈ are hydrogen atoms, may be the same as the cases of Formula 2-1, Formula 2-2, Formula 2-5 and Formula 2-6, where m1, m2, m6, and m8 are 0. If m1, m2, m6, and m8 are integers of 2 or more, each of multiple Rₐ₁, Rₐ₂, Rₐ₆, and Rₐ₈ may all be the same, or at least one selected from among multiple Rₐ₁, Rₐ₂, Rₐ₆, and Rₐ₈ may be different.

In the cases where m5, m7, m9, and m11 are 0, the amine compounds of embodiments may be unsubstituted with Rₐ₅, Rₐ₇, Rₐ₉, and Rₐ₁₁, respectively. The cases where m5, m7, m9, and m11 are 4, and Rₐ₅, Rₐ₇, Rₐ₉, and Rₐ₁₁ are hydrogen atoms, may be the same as the cases of Formula 2-5 to Formula 2-8, where m5, m7, m9, and m11 are 0. If m5, m7, m9, and m11 are integers of 2 or more, each of multiple Rₐ₅, Rₐ₇, Rₐ₉, and Rₐ₁₁ may all be the same, or at least one selected from among multiple Rₐ₅, Rₐ₇, Rₐ₉, and Rₐ₁₁ may be different.

The cases where m3 and m4 are 0, may mean the amine compounds of embodiments unsubstituted with Rₐ₃ and Rₐ₄, respectively. The cases where m3 and m4 are 7, and Rₐ₃ and Rₐ₄ are all hydrogen atoms, may be the same as the cases of Formula 2-3 and Formula 2-4 where m3 and m4 are 0. If m3 and m4 are integers of 2 or more, multiple Rₐ₃ and Rₐ₄ may all be the same, or at least one selected from among multiple Rₐ₃ and Rₐ₄ may be different.

The cases where m10 and m12 are 0, may mean the amine compounds of embodiments unsubstituted with Rₐ₁₀ and Rₐ₁₂, respectively. The cases where m10 and m12 are 11, and Rₐ₁₀ and Rₐ₁₂ are all hydrogen atoms may be the same as the cases of Formula 2-7 and Formula 2-8 where m10 and m12 are 0. If m10 and m12 are integers of 2 or more, multiple Rₐ₁₀ and Rₐ₁₂ may all be the same, or at least one selected from among multiple Rₐ₁₀ and Rₐ₁₂ may be different.

In Formula 2-1 to Formula 2-8, the same description of Rₐ, R_{b}, Ar₁, Ar₂, C1 and C2 referring to Formula 1 and Formula 1-a may be applied.

In an embodiment, the amine compound represented by Formula 1 may be represented by Formula 3-1 or Formula 3-2 below.

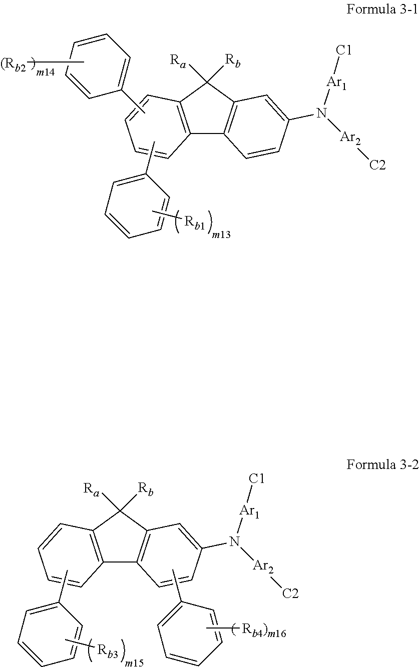

Formula 3-1 and Formula 3-2 represent the structure of Formula 1 where M is represented by Formula 1-a, the carbon position of the first fused ring represented by Formula 1-a to which the nitrogen atom of Formula 1 is bonded is specified, and the number and type of substituents connected with the substituent represented by Formula 1-a are specified. For example, in the structure of Formula 1-a, the number and type of R₁ and R₂ are specified.

Formula 3-1 represents a case where n1 is 2, and n2 is 0 in Formula 1-a, and all substituents represented by R₁ are substituted or unsubstituted phenyl groups. Formula 3-2 represents a case where n1 and n2 are 1 in Formula 1-a, and the substituent represented by R₁ is a substituted or unsubstituted phenyl group, and the substituent represented by R₂ is a substituted or unsubstituted phenyl group.

In Formula 3-1 and Formula 3-2, R_{b1} to R_{b4} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R_{b1} to R_{b4} may be a hydrogen atom.

In Formula 3-1 and Formula 3-2, m13 to m16 are each independently an integer of 0 to 5. If m13 to m16 are 0, the amine compounds of embodiments may be unsubstituted with R_{b1} to R_{b4}, respectively. In Formula 3-1 and Formula 3-2, the cases where m13 to m16 are 5, and R_{b1} to R_{b4} are hydrogen atoms, may be the same as Formula 3-1 and Formula 3-2, where m13 to m16 are 0, respectively. If m13 to m16 are integers of 2 or more, each of multiple R_{b1} to R_{b4} may all be the same, or at least one selected from among multiple R_{b1} to R_{b4} may be different.

In Formula 3-1 and Formula 3-2, the same description of Rₐ, R_{b}, Ar₁, Ar₂, C1 and C2 referring to Formula 1 and Formula 1-a may be applied.

In an embodiment, the amine compound represented by Formula 1 may be represented by any one selected from among Formula 4-1 to Formula 4-15 below.

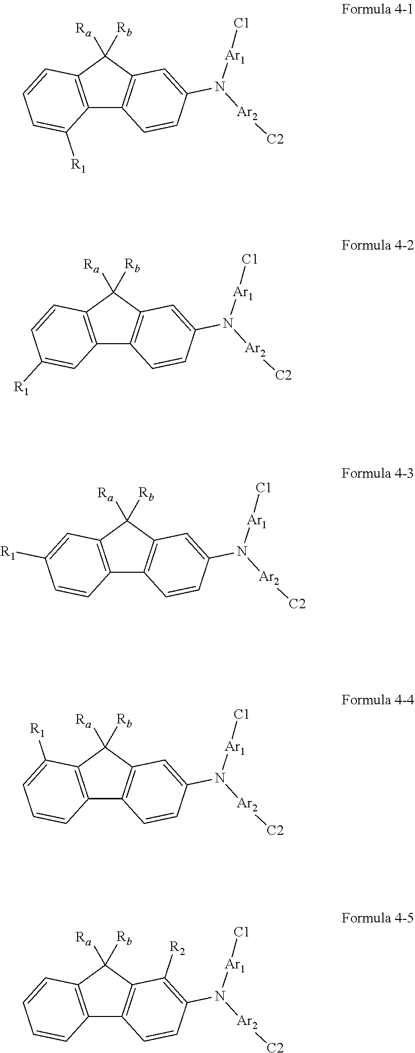

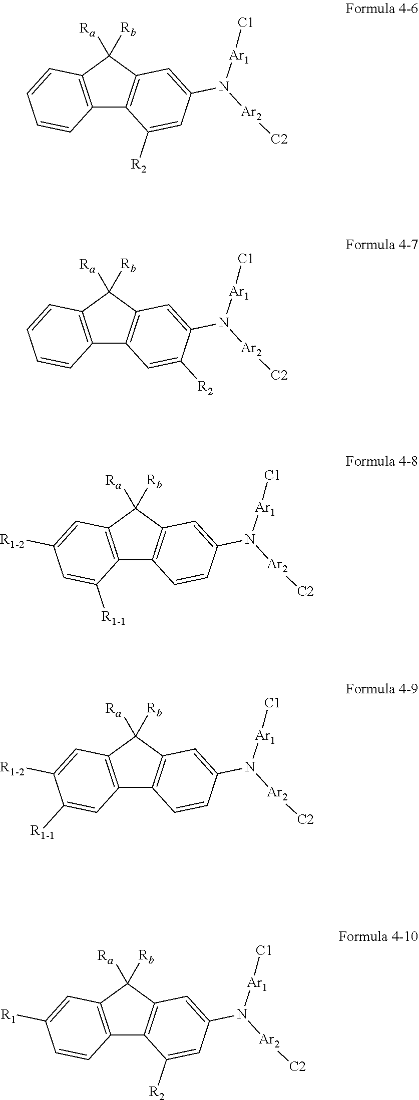

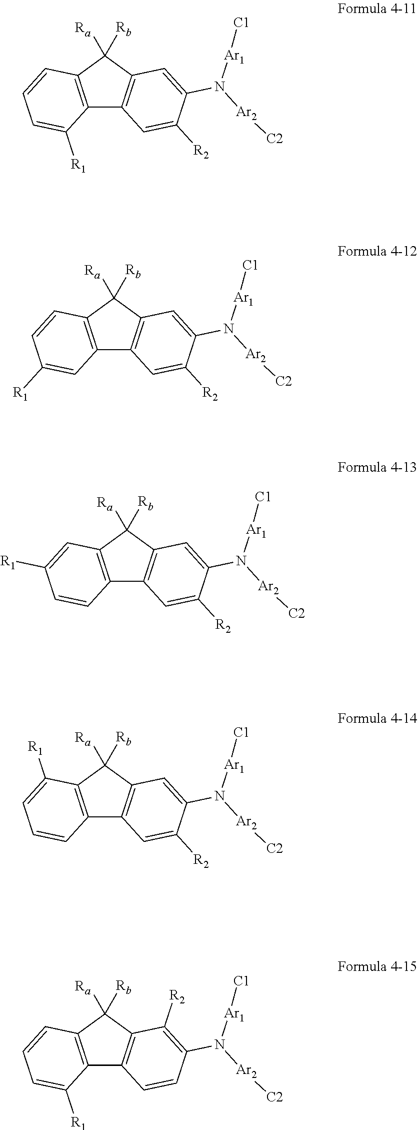

Formula 4-1 to Formula 4-15 represent the structure of Formula 1 where m is represented by Formula 1-a, and the number and connecting position of substituents connected with the first fused ring represented by Formula 1-a are specified. For example, in the first fused ring structure represented by Formula 1-a, the number and connecting positions of R₁ and R₂ are specified.

In Formula 4-8 and Formula 4-9, R₁₋₁ and R₁₋₂ are each independently a substituted or unsubstituted aryl group of 6 to 30 ring-forming carbon atoms, or a substituted or unsubstituted heteroaryl group of 2 to 30 ring-forming carbon atoms. For example, R₁₋₁ and R₁₋₂ may be each independently a substituted or unsubstituted phenyl group, a substituted or unsubstituted biphenyl group, or a substituted or unsubstituted naphthyl group.

In Formula 4-1 to Formula 4-15, the same description of R₁, R₂, Rₐ, R_{b}, Ar₁, Ar₂, C1, and C2 referring to Formula 1 and Formula 1-a may be applied.

In an embodiment, the amine compound represented by Formula 1 may be represented by any one selected from among Formula 5-1 to Formula 5-3 below.

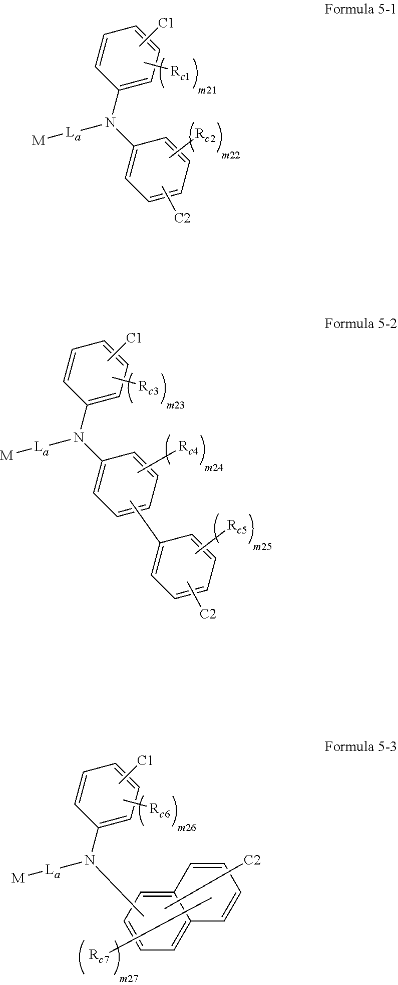

Formula 5-1 to Formula 5-3 represent Formula 1 where Ar₁ and Ar₂ are specified as particular structures. Formula 5-1 represents Formula 1 where Ar₁ and Ar₂ are each independently a substituted or unsubstituted phenylene group. Formula 5-2 represents Formula 1 where Ar₁ is a substituted or unsubstituted phenylene group, and Ar₂ is a substituted or unsubstituted divalent biphenyl group. Formula 5-3 represents Formula 1 where Ar₁ is a substituted or unsubstituted phenylene group, and Ar₂ is a substituted or unsubstituted divalent naphthyl group.

C1 and C2 included in the amine compound of an embodiment may not be directly bonded to the amine group but may be bonded to the nitrogen atom of the amine group via Ar₁ and Ar₂. In an embodiment, Ar₁ and Ar₂ connecting C1 and C2 to the nitrogen atom of the amine group may be each independently a substituted or unsubstituted phenylene group, a substituted or unsubstituted divalent biphenyl group, or a substituted or unsubstituted divalent naphthyl group.

In Formula 5-1 to Formula 5-3, R_{c1} to R_{c7} may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In an embodiment, R_{c1} to R_{c7} may be each independently a hydrogen atom or a substituted or unsubstituted phenyl group.

In Formula 5-1 to Formula 5-3, m21 to m26 are each independently an integer of 0 to 4, and m27 is an integer of 0 to 6.

If m21 to m26 are 0, the amine compounds of embodiments may be unsubstituted with R_{c1} to R_{c6}, respectively. The cases where m21 to m26 are 4, and R_{c1} to R_{c6} are hydrogen atoms, may be the same as Formula 5-1 to Formula 5-3, where m21 to m26 are 0. If m21 to m26 are integers of 2 or more, each of multiple R_{c1} to R_{c6} may be the same, or at least one selected from among multiple R_{c1} to R_{c6} may be different.

If m27 is 0, the amine compound of an embodiment may be unsubstituted with R_{c7}. Formula 4-3 where m27 is 6, and all R_{c7} are hydrogen atoms, may be the same as Formula 5-3 where m27 is 0. If m27 is an integer of 2 or more, multiple R_{c7} may all be the same, or at least one selected from among multiple R_{c7} may be different.

The same description of M, Lₐ, C1, and C2 referring to Formula 1 may be applied.

In an embodiment, the amine compound represented by Formula 1 may be represented by any one selected from among Formula 6-1 to Formula 6-8 below.

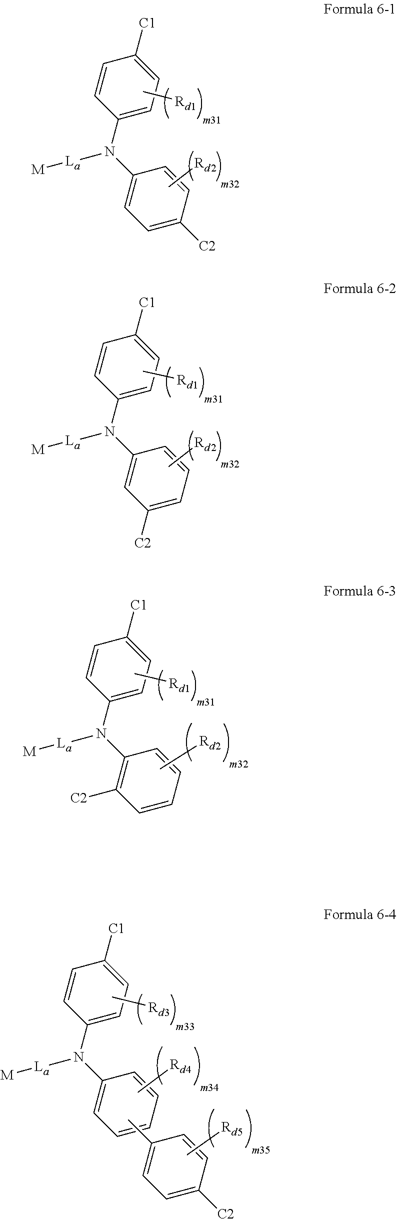

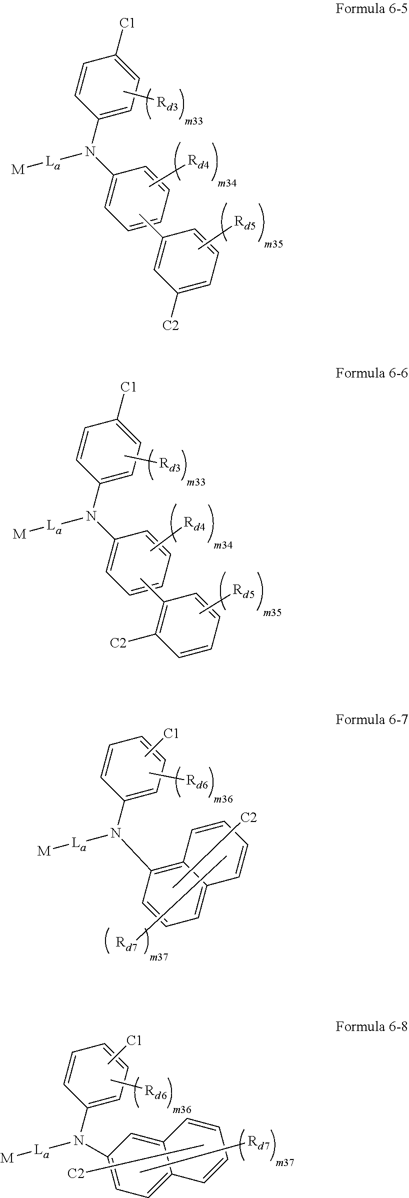

Formula 6-1 to Formula 6-8 represent Formula 1 where Ar₁ and Ar₂ are specified as particular structures, and the positions of C1 and C2 connected with Ar₁ and Ar₂, respectively, are specified.

In Formula 6-1 to Formula 6-8, R_{d1} to R_{d7} may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R_{d1} to R_{d7} may be each independently a hydrogen atom, or a substituted or unsubstituted phenyl group.

In Formula 6-1 to Formula 6-8, m31 to m36 are each independently an integer of 0 to 4, and m37 is an integer of 0 to 6.

If m31 to m36 are 0, the amine compounds of embodiments may be unsubstituted with R_{d1} to R_{d6}, respectively. The cases where m31 to m36 are 4, and R_{d1} to R_{d6} are all hydrogen atoms, may be the same as Formula 6-1 to Formula 6-8 where m31 to m36 are 0. If m31 to m36 are integers of 2 or more, each of multiple R_{d1} to R_{d6} may be the same, or at least one of multiple R_{d1} to R_{d6} may be different.

If m37 is 0, the amine compound of an embodiment may be unsubstituted with R_{d7}. Formula 6-7 and Formula 6-8, where m37 is 6, and all R_{d7} are hydrogen atoms may be the same as Formula 6-7 and Formula 6-8, where m37 is 0. If m37 is an integer of 2 or more, each of multiple R_{d7} may be the same, or at least one selected from among multiple R_{d7} may be different.

The same description of M, Lₐ, C1, and C2 referring to Formula 1 may be applied.

In an embodiment, C1 and C2 may be each independently represented by any one selected from among Formula 7-1 to Formula 7-8 below.

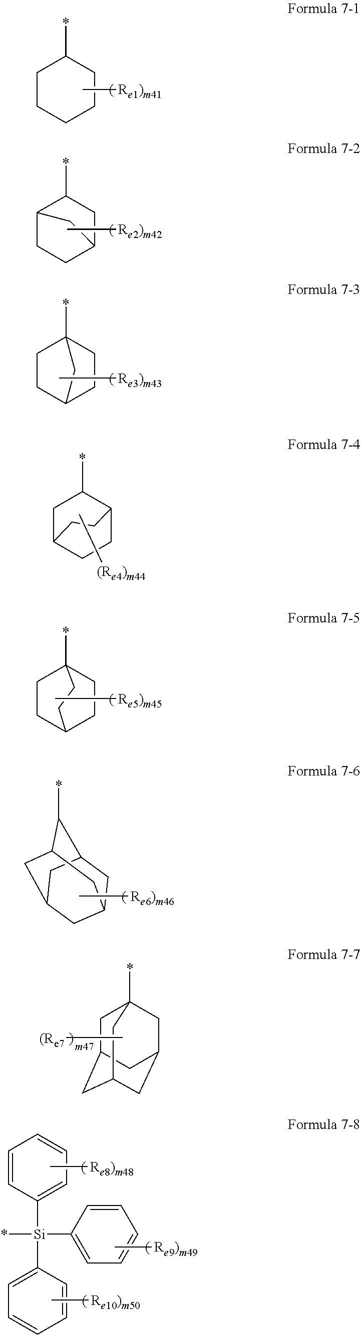

In Formula 7-1 to Formula 7-8, Rₑ₁ to Rₑ₁₀ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Rₑ₁ to Rₑ₁₀ may be a hydrogen atom.

In Formula 7-1 to Formula 7-8, m41 to m43 are each independently an integer of 0 to 11, m44 and m45 are each independently an integer of 0 to 13, m46 and m47 are each independently an integer of 0 to 15, and m48 to m50 are each independently an integer of 0 to 5.

If m41 to m43 are 0, the amine compounds of embodiments may be unsubstituted with Rₑ₁ to Rₑ₃, respectively. The cases where m41 to m43 are 11, and Rₑ₁ to Rₑ₃ are hydrogen atoms, may be the same as Formula 7-1 to Formula 7-3 where m41 to m43 are 0, respectively. If m41 to m43 are integers of 2 or more, each of multiple Rₑ₁ to Rₑ₃ may all be the same, or at least one selected from among multiple Rₑ₁ to Rₑ₃ may be different.

If m44 and m45 are 0, the amine compounds of embodiments may be unsubstituted with Rₑ₄ and Rₑ₅, respectively. The cases where m44 and m45 are 13, and Rₑ₄ and Rₑ₅ are hydrogen atoms, may be the same as Formula 7-4 and Formula 7-5 where m44 and m45 are 0, respectively. If m44 and m45 are integers of 2 or more, each of multiple Rₑ₄ and Rₑ₅ may all be the same, or at least one selected from among multiple Rₑ₄ and Rₑ₅ may be different.

If m46 and m47 are 0, the amine compounds of embodiments may be unsubstituted with Rₑ₆ and Rₑ₇, respectively. The cases where m46 and m47 are 15, and Rₑ₆ and Rₑ₇ are hydrogen atoms, may be the same as Formula 7-6 and Formula 7-7 where m46 and m47 are 0, respectively. If m46 and m47 are integers of 2 or more, each of multiple Rₑ₆ and Rₑ₇ may all be the same, or at least one selected from among multiple Rₑ₆ and Rₑ₇ may be different.

If m48 to m50 are 0, the amine compounds of embodiments may be unsubstituted with Rₑ₈ to Rₑ₁₀, respectively. The cases where m48 to m50 are 5, and Rₑ₈ to Rₑ₁₀ are hydrogen atoms, may be the same as Formula 7-8 where m48 to m50 are 0. If m48 to m50 are integers of 2 or more, each of multiple Rₑ₈ to Rₑ₁₀ may all be the same, or at least one selected from among multiple Rₑ₈ to Rₑ₁₀ may be different.

In an embodiment, the amine compound represented by Formula 1 may be represented by any one selected from among Formula 8-1 to Formula 8-5 below.

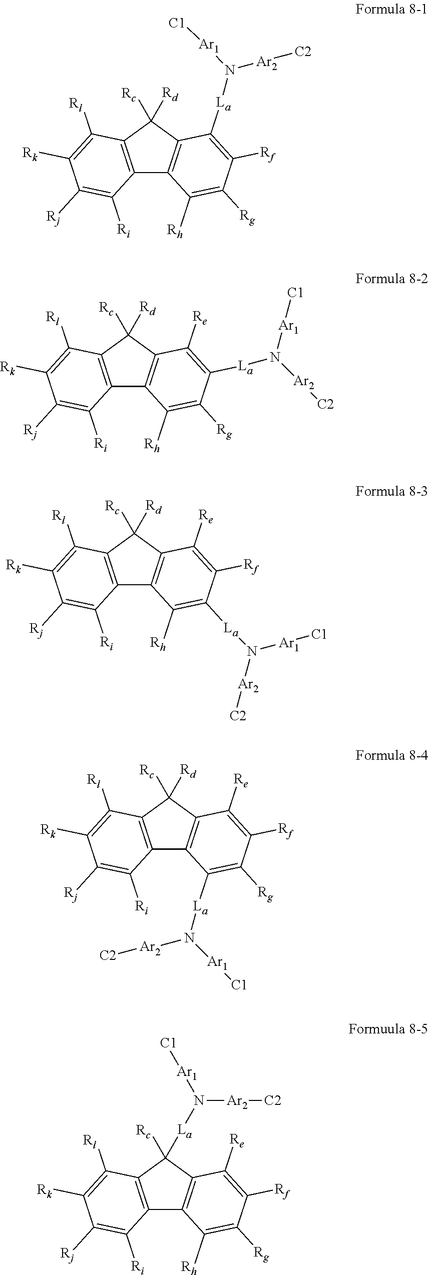

Formula 8-1 to Formula 8-5 represent the structure of Formula 1 where M is represented by Formula 1-b, and the position where the nitrogen atom of Formula 1 is connected with the substituent represented by Formula 1-b is specified.

In Formula 8-1 to Formula 8-5, at least one selected from among C1 and C2 may be a substituted or unsubstituted bicycloheptanyl group.

In Formula 8-1 to Formula 8-5, the same description of Ar₁, Ar₂, Lₐ, C1, C2, and R_{c} to Rₗ referring to Formula 1 and Formula 1-b may be applied.

In an embodiment, the amine compound represented by Formula 1 may be represented by any one selected from among Formula 9-1 to Formula 9-4 below.

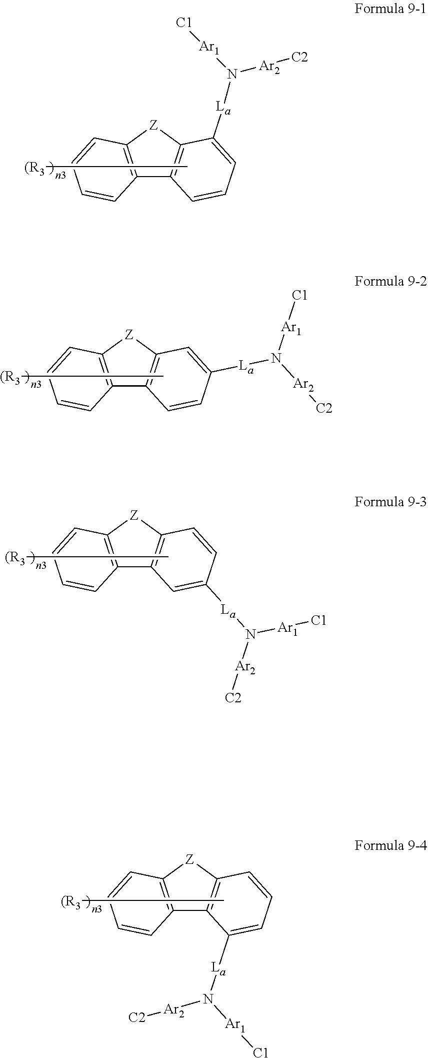

Formula 9-1 to Formula 9-4 represent the structure of Formula 1 where M is represented by Formula 1-c, and the position where the nitrogen atom of Formula 1 is connected with the substituent represented by Formula 1-c is specified.

In Formula 9-1 to Formula 9-4, at least one selected from among C1 and C2 may be a substituted or unsubstituted bicycloheptanyl group.

In Formula 9-1 to Formula 9-4, the same description of Z, R₃, Ar₁, Ar₂, Lₐ, n3, C1, and C2 referring to Formula 1 and Formula 1-c may be applied.

In the amine compound of an embodiment, represented by Formula 1, Ar₁ and Ar₂ may be each independently represented by any one selected from among Formulae L-1 to L-15 below.

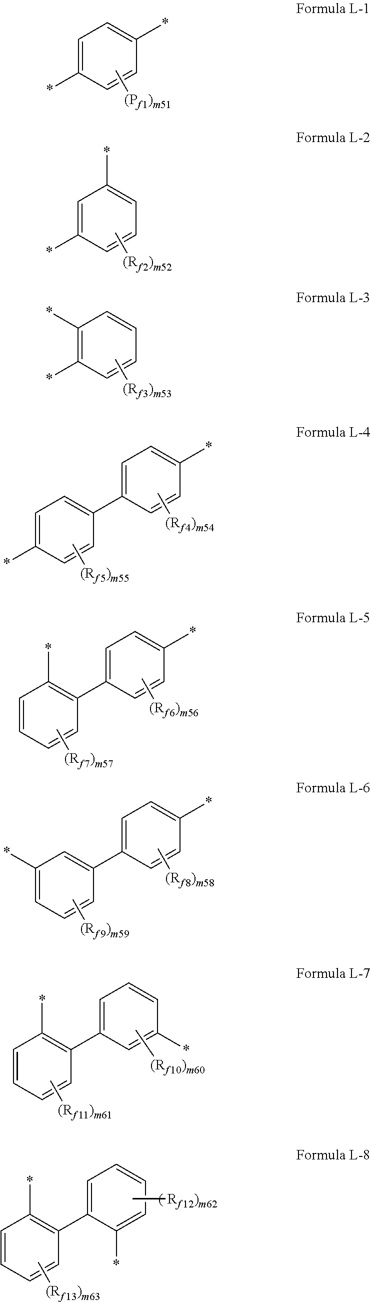

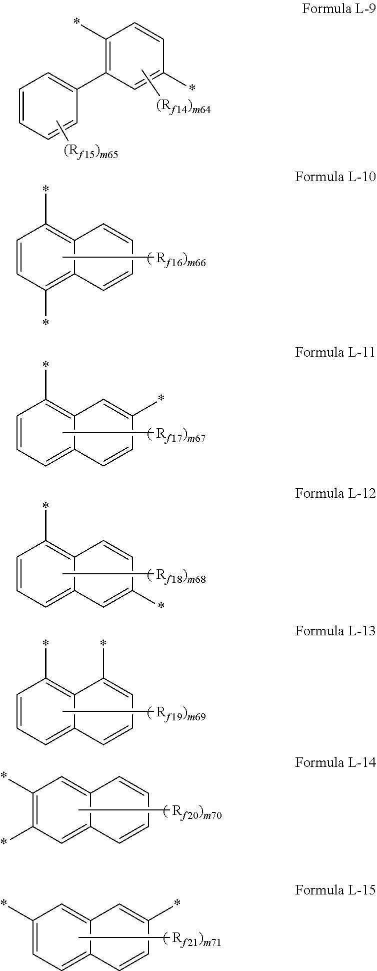

In Formula L-1 to Formula L-15, R_{f1} to R_{f21} may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula L-1 to Formula L-15, m51 to m63 are each independently an integer of 0 to 4, m64 is an integer of 0 to 3, m65 is an integer of 0 to 5, and m66 to m71 are each independently an integer of 0 to 6.

If m51 to m63 are 0, the amine compounds of embodiments may be unsubstituted with R_{f1} to R_{f13}. The cases where m51 to m63 are 4, and R_{f1} to R_{f13} are all hydrogen atoms, may be the same as the cases where m51 to m63 are 0. If m51 to m63 are integers of 2 or more, each of multiple R_{f1} to R_{f13} may be the same, or at least one of multiple R_{f1} to R_{f13} may be different.

If m64 is 0, the amine compound of an embodiment may be unsubstituted with R_{f14}. The case where m64 is 3, and R_{f14} are all hydrogen atoms, may be the same as the case where m64 is 0. If m64 is an integer of 2 or more, multiple R_{f14} may all be the same, or at least one of multiple R_{f14} may be different.

If m65 is 0, the amine compound of an embodiment may be unsubstituted with R_{f15}. The case where m65 is 5, and R_{f15} are all hydrogen atoms, may be the same as the case where m65 is 0. If m65 is an integer of 2 or more, multiple R_{f15} may all be the same, or at least one of multiple R_{f15} may be different.

If m66 to m71 are 0, the amine compounds of embodiments may be unsubstituted with R_{f16} to R_{f21}, respectively. The cases where m66 to m71 are 6, and R_{f16} to R_{f21} are all hydrogen atoms, may be the same as the cases where m66 to m71 are 0. If m66 to m71 are integers of 2 or more, each of multiple R_{f16} to R_{f21} may be the same, or at least one of multiple R_{f16} to R_{f21} may be different.

In an embodiment, the amine compound represented by Formula 1 may be represented by any one selected from among the compounds in Compound Group 1 below. The hole transport region HTR may include at least one selected from the compounds represented in Compound Group 1 below.

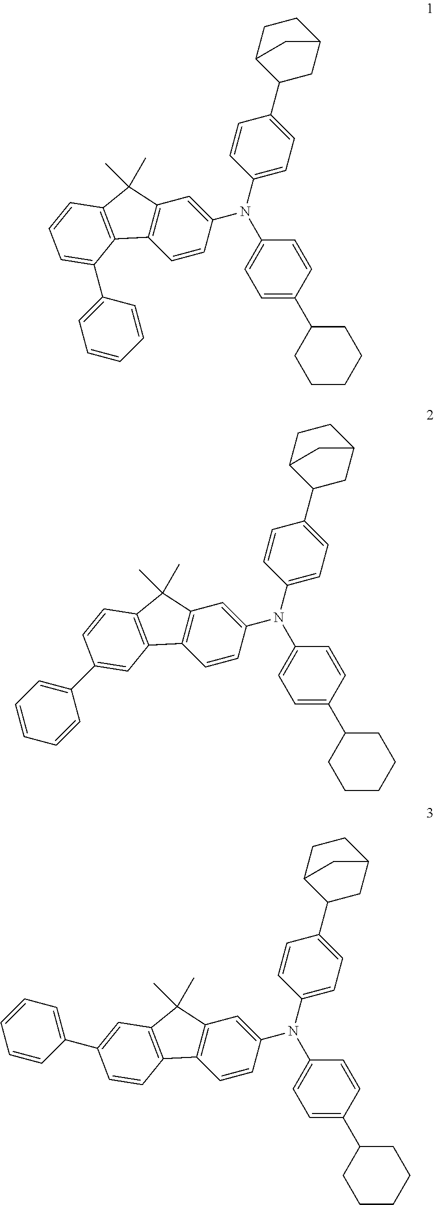

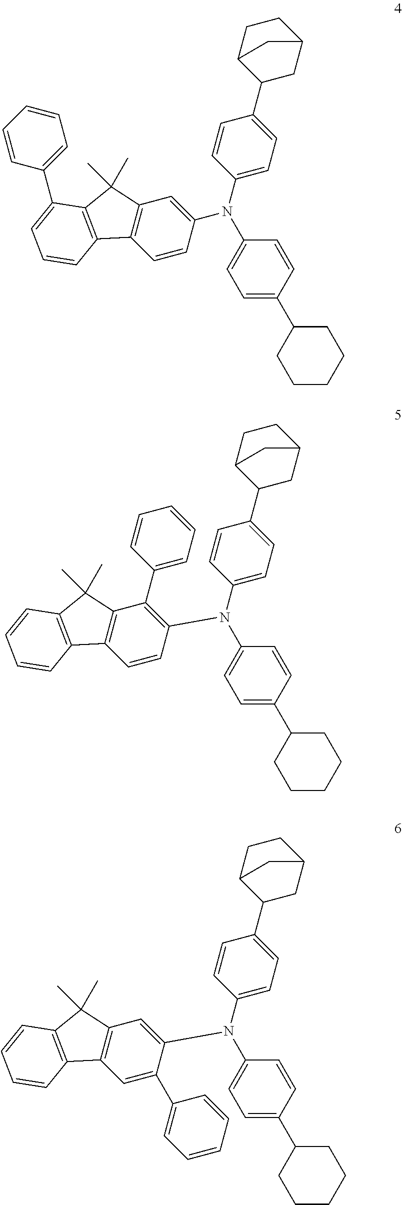

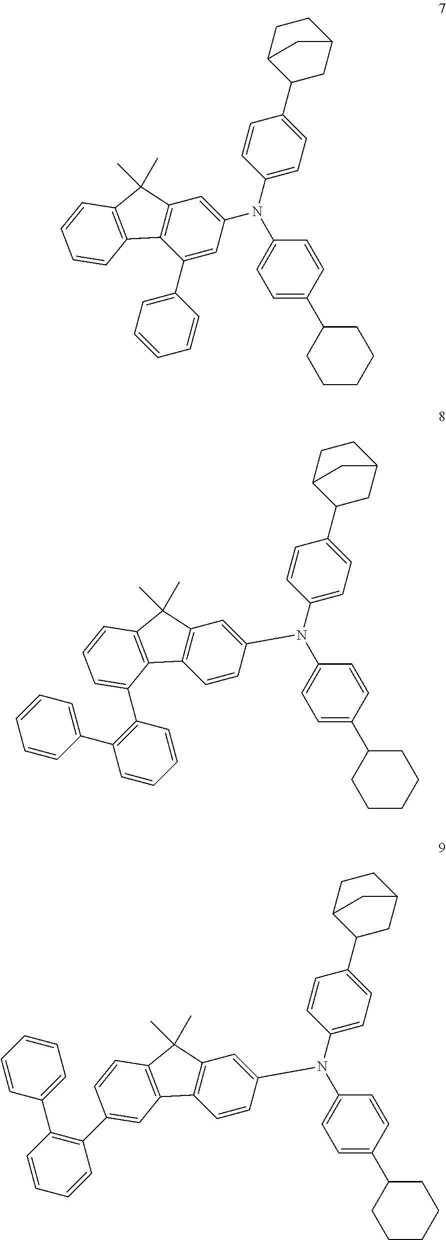 [SMILES: CC1(C)c2ccccc2-c2c(-c3ccccc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4CC5CCC4C5)cc3)cc21]

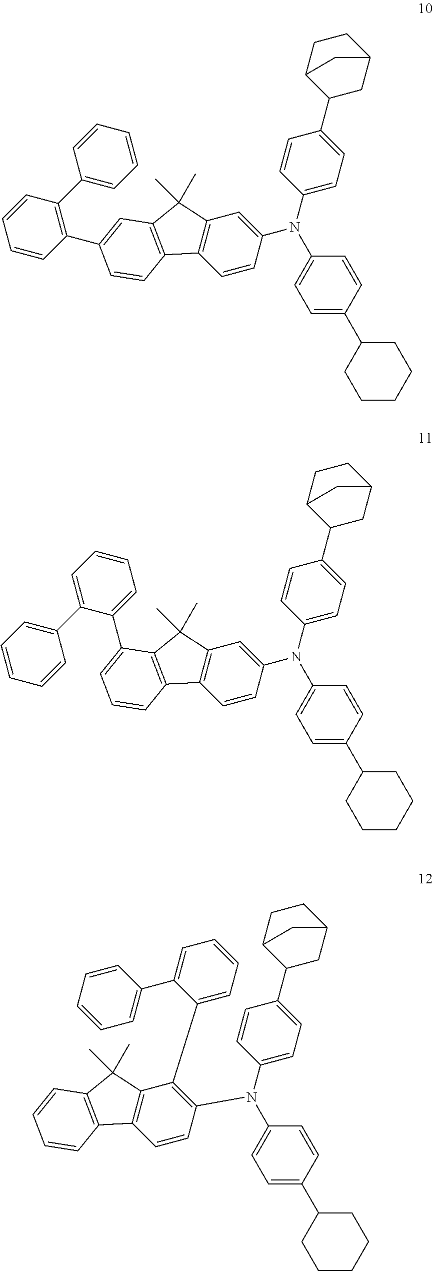 [SMILES: CC1(C)c2cc(-c3ccccc3-c3ccccc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4C5)cc3)c(-c3ccccc3-c3ccccc3)c21]

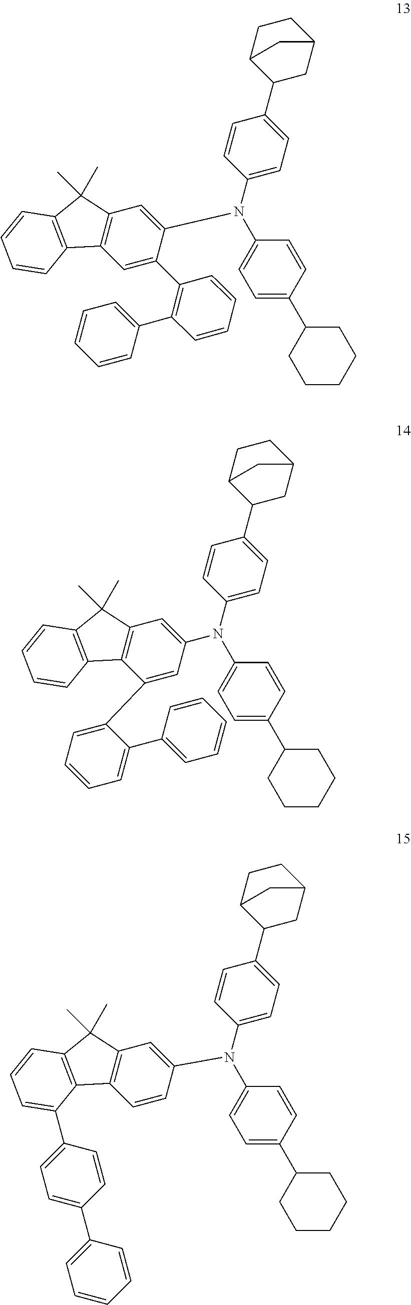 [SMILES: CC1(C)c2ccccc2-c2cc(-c3ccccc3-c3ccccc3)c(N(c3ccc(C4CCCCC4)cc3)c3ccc(C4CC5CCC4C5)cc3)cc21 ; CC1(C)c2ccccc2-c2c(-c3ccccc3-c3ccccc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(-c4ccccc4)cc3)cccc21]

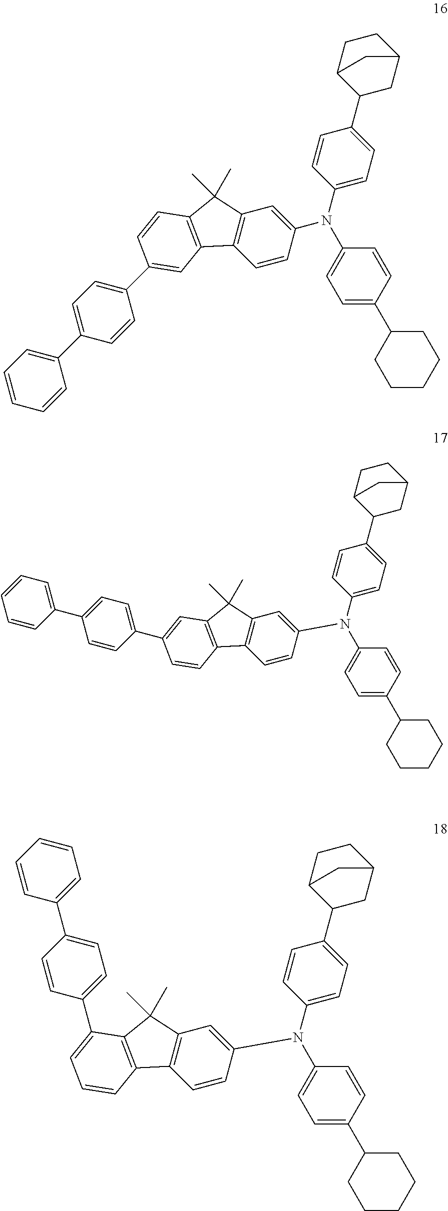 [SMILES: CC1(C)c2ccc(-c3ccc(-c4ccccc4)cc3)cc2-c2ccc(N(c3ccc(C4CCCCC4)cc3)c3ccc(C4CC5CCC4C5)cc3)cc21 ; CC1(C)c2cc(-c3ccc(-c4ccccc4)cc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc(-c4ccccc4)cc3)c21]

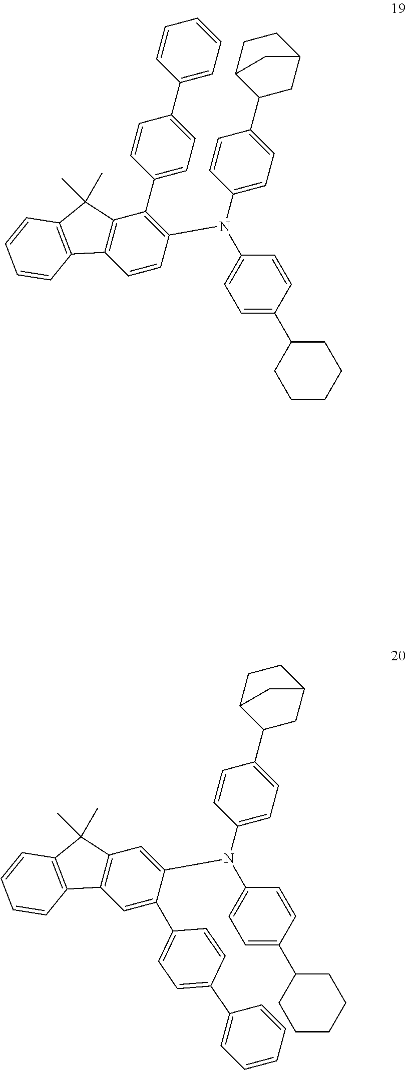 [SMILES: CC1(C)c2ccccc2-c2ccc(N(c3ccc(C4CCCCC4)cc3)c3ccc(C4CC5CCC4C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4CC5CCC4C5)cc3)cc21]

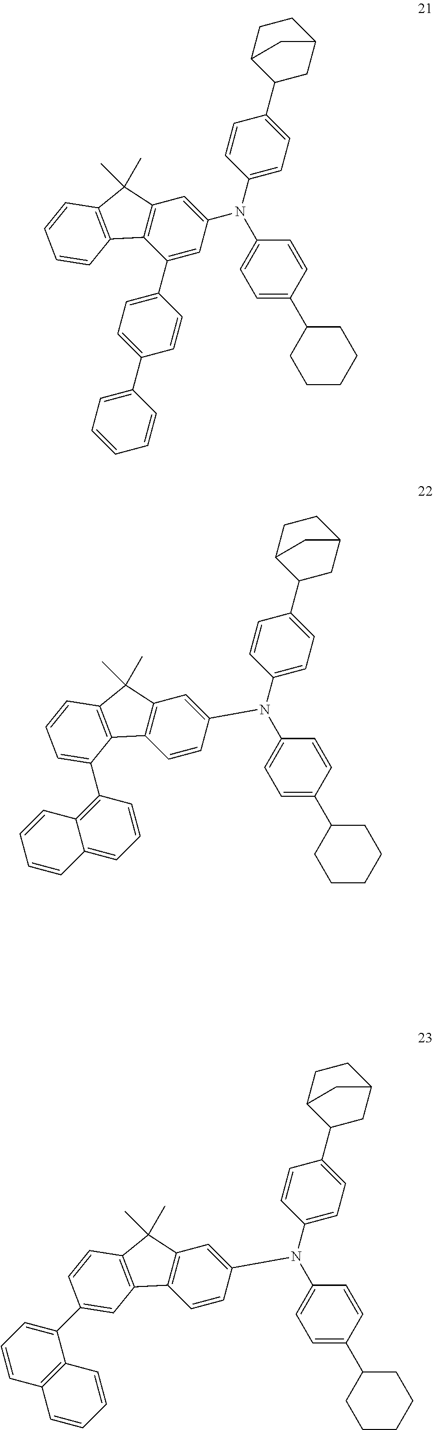 [SMILES: CC1(C)c2ccccc2-c2c(-c3ccc(-c4ccccc4)cc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CC5CCC4C5)cc3)cc21]

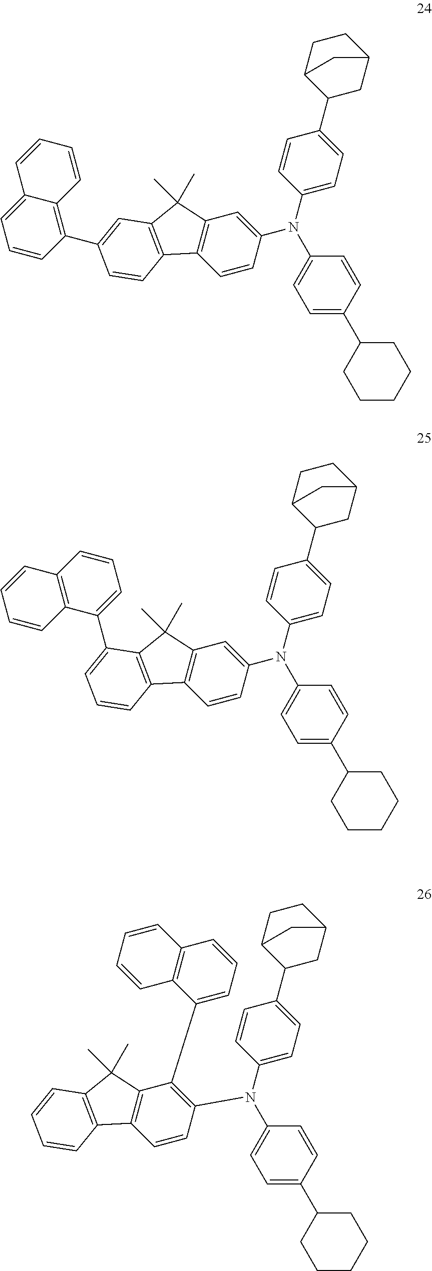 [SMILES: CC1(C)c2cc(-c3cccc4ccccc34)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3cccc4ccccc34)c21 ; CC1(C)c2ccccc2-c2ccc(N(c3ccc(C4CCCCC4)cc3)c3ccc(C4CC5CCC4C5)cc3)c(-c3cccc4ccccc34)c21]

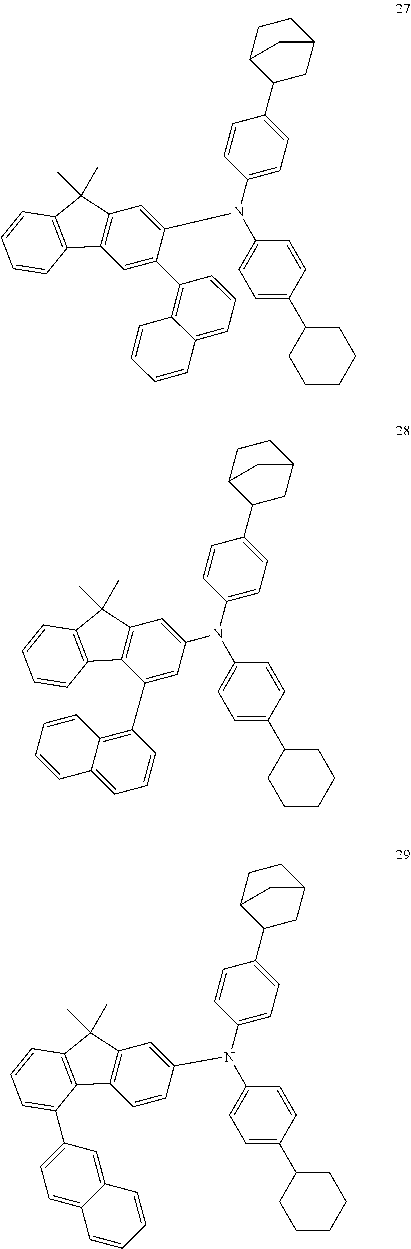 [SMILES: CC1(C)c2ccccc2-c2cc(-c3cccc4ccccc34)c(N(c3ccc(C4CCCCC4)cc3)c3ccc(C4CC5CCC4C5)cc3)cc21 ; CC1(C)c2ccccc2-c2c(-c3cccc4ccccc34)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc4ccccc4c3)cccc21]

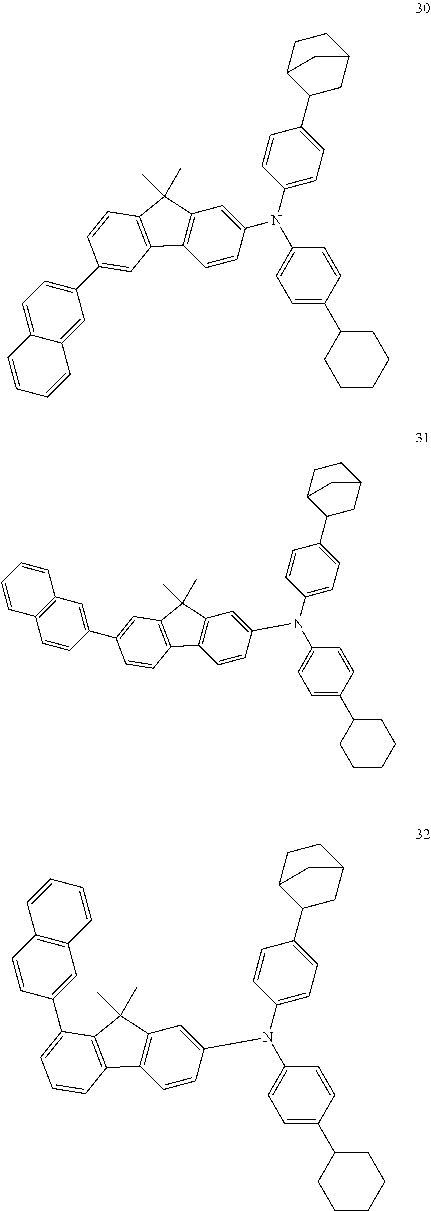 [SMILES: CC1(C)c2ccc(-c3ccc4ccccc4c3)cc2-c2ccc(N(c3ccc(C4CCCCC4)cc3)c3ccc(C4CC5CCC4C5)cc3)cc21 ; CC1(C)c2cc(-c3ccc4ccccc4c3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc4ccccc4c3)c21]

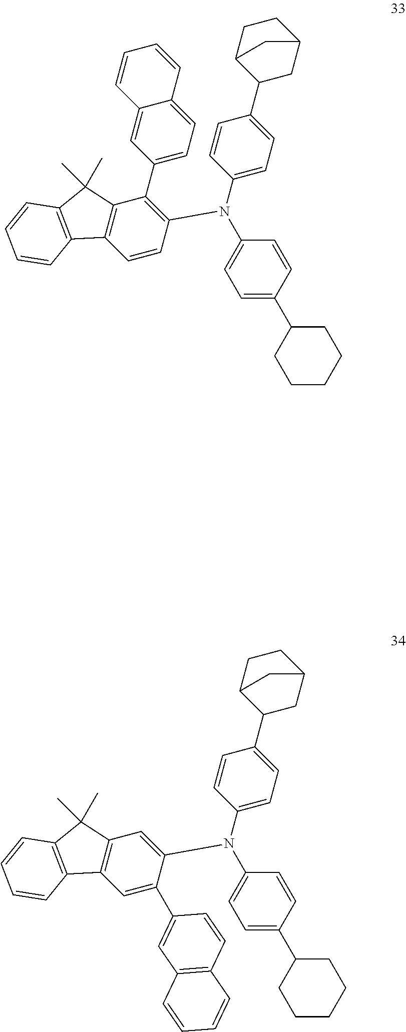 [SMILES: CC1(C)c2ccccc2-c2ccc(N(c3ccc(C4CCCCC4)cc3)c3ccc(C4CC5CCC4C5)cc3)c(-c3ccc4ccccc4c3)c21 ; CC1(C)c2ccccc2-c2cc(-c3ccc4ccccc4c3)c(N(c3ccc(C4CCCCC4)cc3)c3ccc(C4CC5CCC4C5)cc3)cc21]

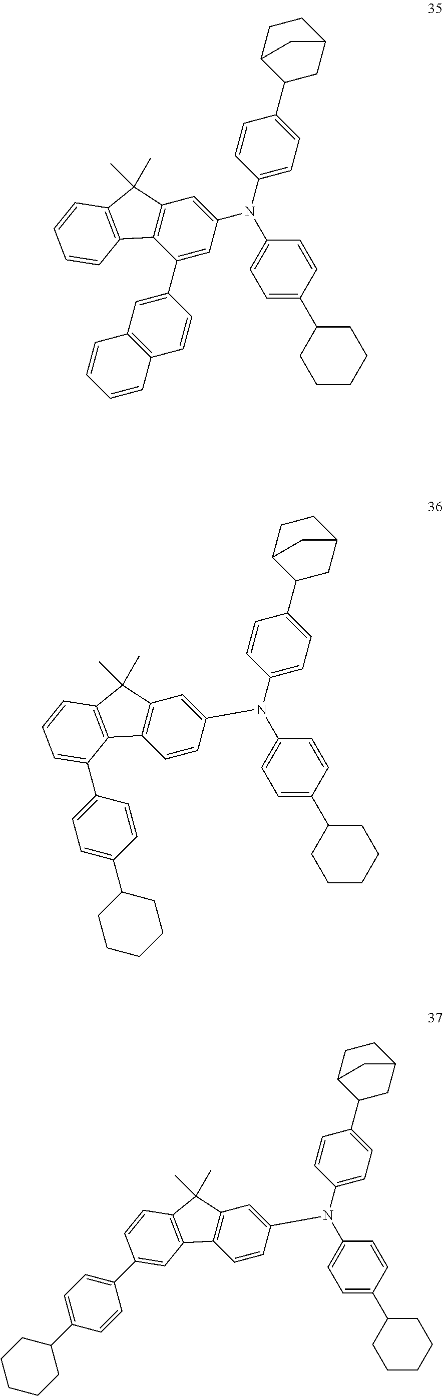 [SMILES: CC1(C)c2ccccc2-c2c(-c3ccc4ccccc4c3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CCCCC4)cc3)c3ccc(C4CC5CCC4C5)cc3)cc21]

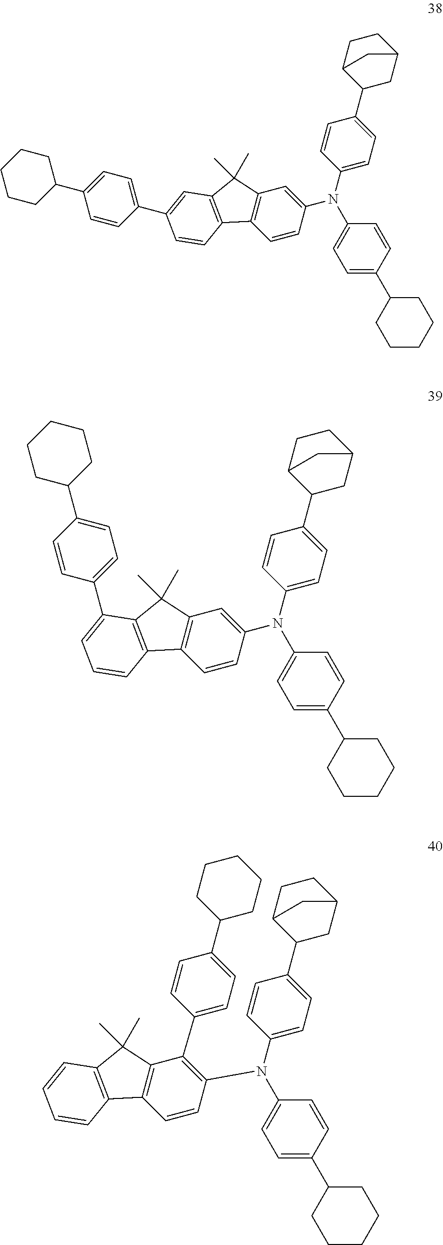 [SMILES: CC1(C)c2cc(-c3ccc(C4CCCCC4)cc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc(C4CCCCC4)cc3)c21 ; CC1(C)c2ccccc2-c2ccc(N(c3ccc(C4CCCCC4)cc3)c3ccc(C4CC5CCC4C5)cc3)c(-c3ccc(C4CCCCC4)cc3)c21]

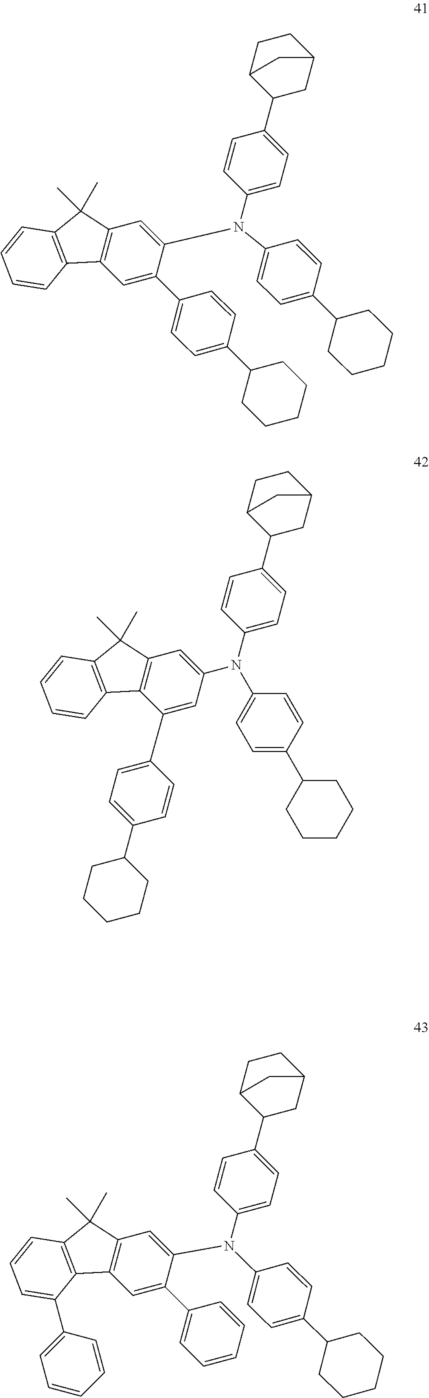 [SMILES: CC1(C)c2ccccc2-c2cc(-c3ccc(C4CCCCC4)cc3)c(N(c3ccc(C4CCCCC4)cc3)c3ccc(C4CC5CCC4C5)cc3)cc21 ; CC1(C)c2ccccc2-c2c(-c3ccc(C4CCCCC4)cc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)c(-c3ccccc3)cc2-c2c(-c3ccccc3)cccc21]

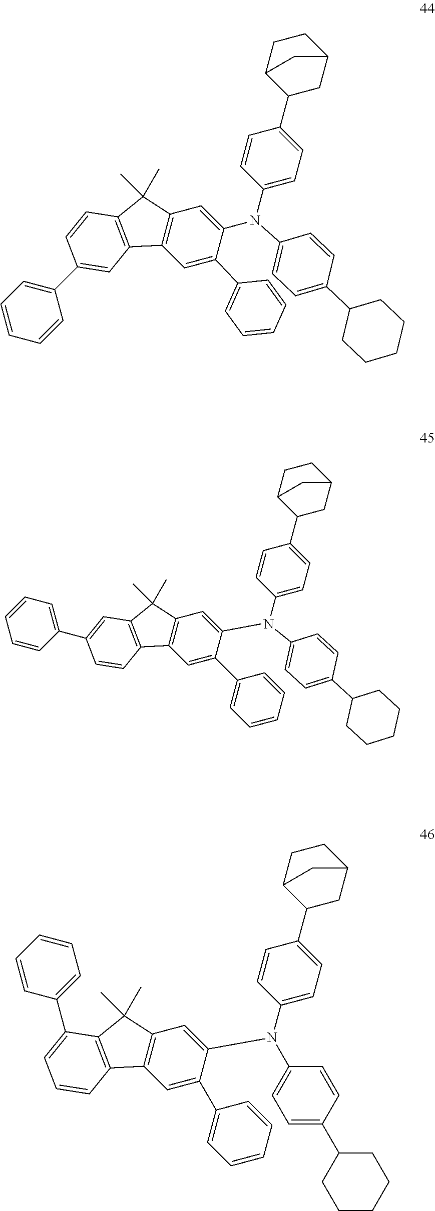 [SMILES: CC1(C)c2ccc(-c3ccccc3)cc2-c2cc(-c3ccccc3)c(N(c3ccc(C4CCCCC4)cc3)c3ccc(C4CC5CCC4C5)cc3)cc21 ; CC1(C)c2cc(-c3ccccc3)ccc2-c2cc(-c3ccccc3)c(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)c(-c3ccccc3)cc2-c2cccc(-c3ccccc3)c21]

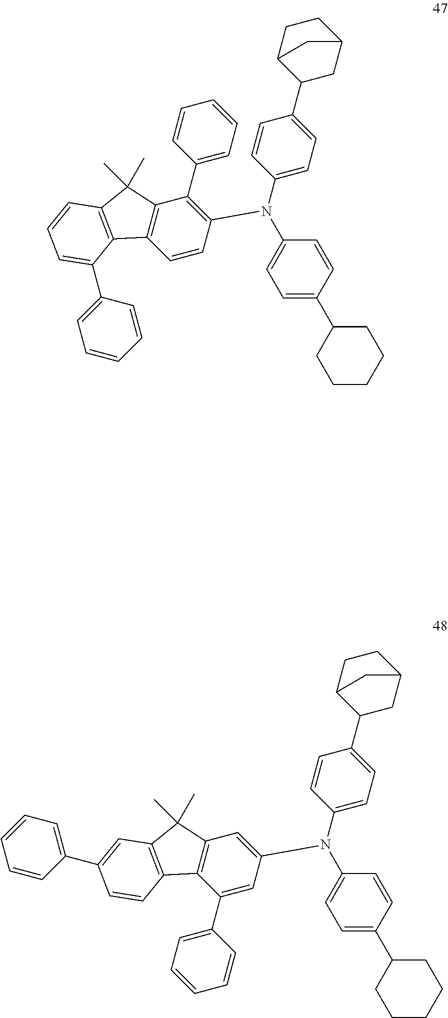 [SMILES: CC1(C)c2cccc(-c3ccccc3)c2-c2ccc(N(c3ccc(C4CCCCC4)cc3)c3ccc(C4CC5CCC4C5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4CC5CCC4C5)cc3)cc21]

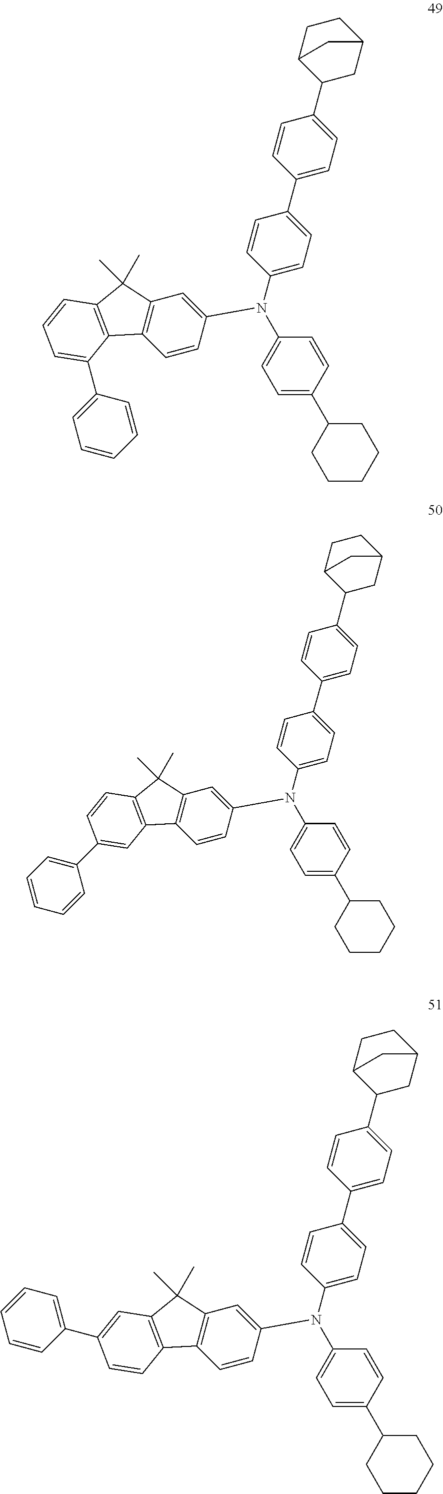 [SMILES: CC1(C)c2cc(N(c3ccc(-c4ccc(C5CC6CCC5C6)cc4)cc3)c3ccc(C4CCCCC4)cc3)ccc2-c2c(-c3ccccc3)cccc21 ; CC1(C)c2ccc(-c3ccccc3)cc2-c2ccc(N(c3ccc(-c4ccc(C5CC6CCC5C6)cc4)cc3)c3ccc(C4CCCCC4)cc3)cc21 ; CC1(C)c2cc(-c3ccccc3)ccc2-c2ccc(N(c3ccc(-c4ccc(C5CC6CCC5C6)cc4)cc3)c3ccc(C4CCCCC4)cc3)cc21]

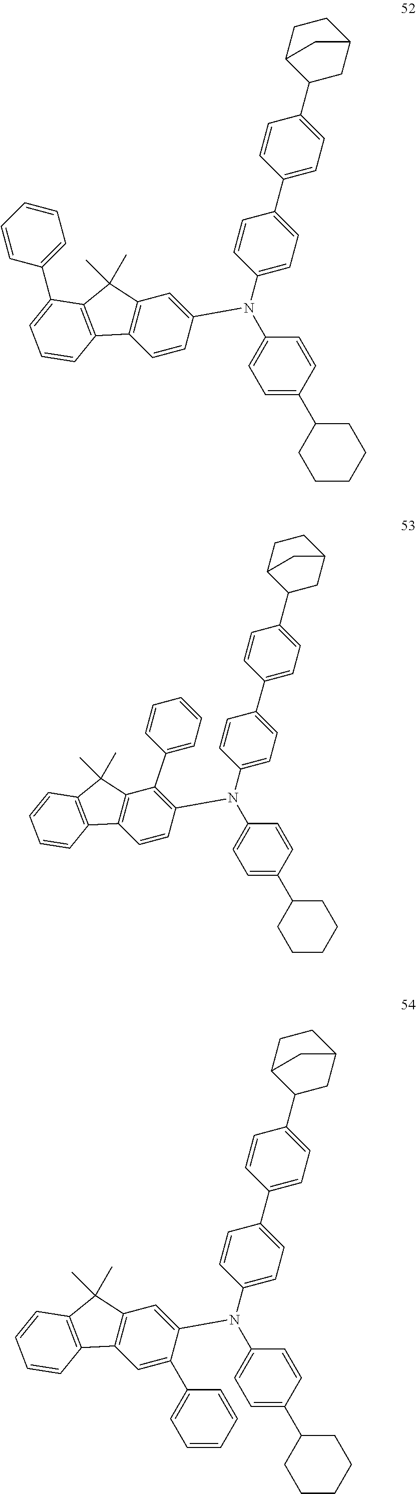 [SMILES: CC1(C)c2cc(N(c3ccc(-c4ccc(C5CC6CCC5C6)cc4)cc3)c3ccc(C4CCCCC4)cc3)ccc2-c2cccc(-c3ccccc3)c21 ; CC1(C)c2ccccc2-c2ccc(N(c3ccc(-c4ccc(C5CC6CCC5C6)cc4)cc3)c3ccc(C4CCCCC4)cc3)c(-c3ccccc3)c21 ; CC1(C)c2ccccc2-c2cc(-c3ccccc3)c(N(c3ccc(-c4ccc(C5CC6CCC5C6)cc4)cc3)c3ccc(C4CCCCC4)cc3)cc21]

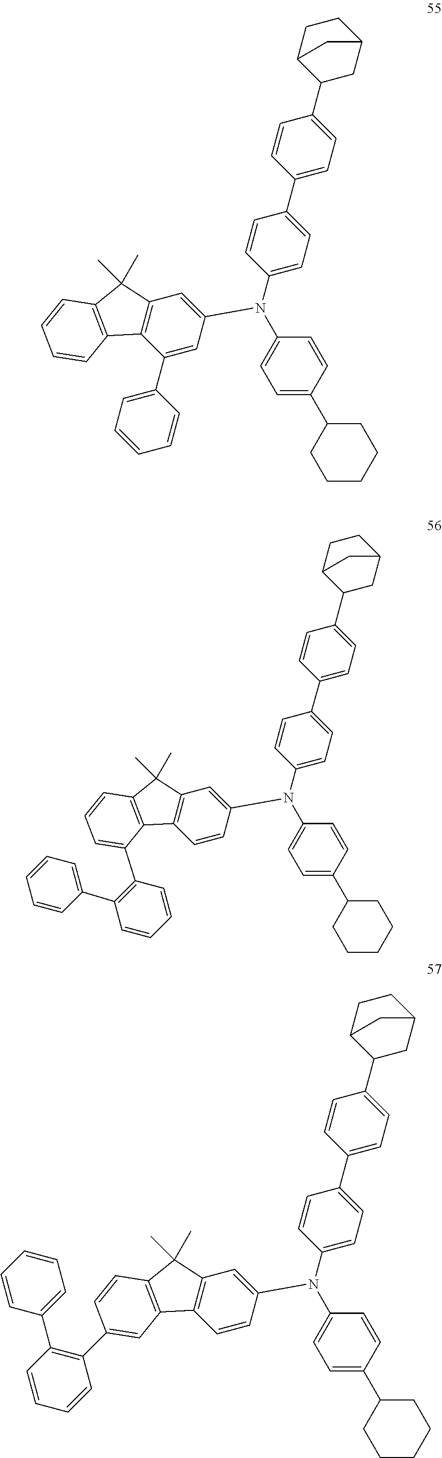

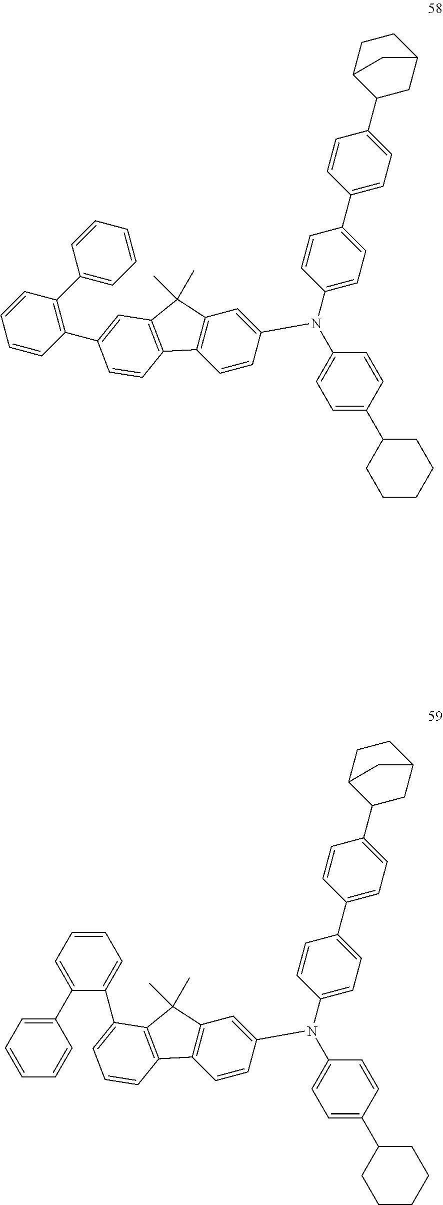 [SMILES: CC1(C)c2cc(-c3ccccc3-c3ccccc3)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3ccccc3-c3ccccc3)c21]

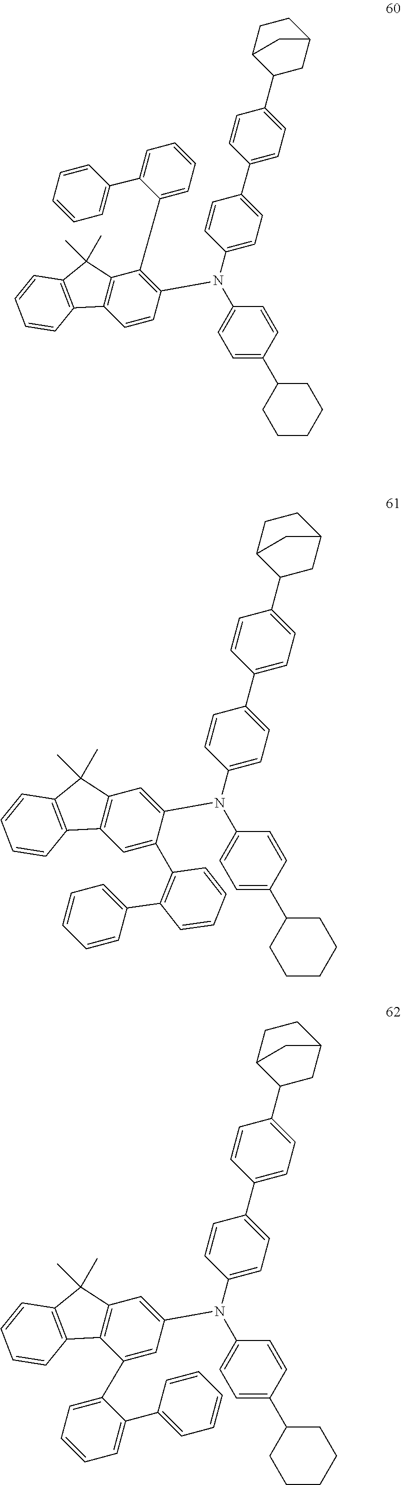 [SMILES: CC1(C)c2ccccc2-c2ccc(N(c3ccc(-c4ccc(C5CC6CCC5C6)cc4)cc3)c3ccc(C4CCCCC4)cc3)c(-c3ccccc3-c3ccccc3)c21 ; CC1(C)c2ccccc2-c2cc(-c3ccccc3-c3ccccc3)c(N(c3ccc(-c4ccc(C5CC6CCC5C6)cc4)cc3)c3ccc(C4CCCCC4)cc3)cc21 ; CC1(C)c2ccccc2-c2c(-c3ccccc3-c3ccccc3)cc(N(c3ccc(-c4ccc(C5CC6CCC5C6)cc4)cc3)c3ccc(C4CCCCC4)cc3)cc21]

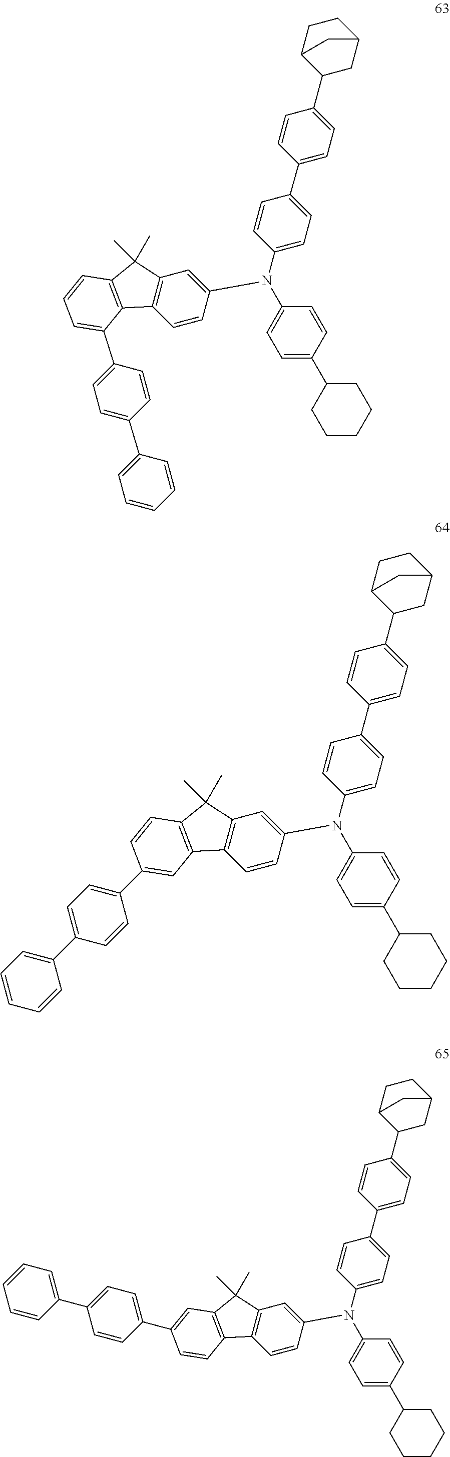 [SMILES: CC1(C)c2cc(N(c3ccc(-c4ccc(C5CC6CCC5C6)cc4)cc3)c3ccc(C4CCCCC4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(-c4ccc(C5CC6CCC5C6)cc4)cc3)c3ccc(C4CCCCC4)cc3)cc21 ; CC1(C)c2cc(-c3ccc(-c4ccccc4)cc3)ccc2-c2ccc(N(c3ccc(-c4ccc(C5CC6CCC5C6)cc4)cc3)c3ccc(C4CCCCC4)cc3)cc21]

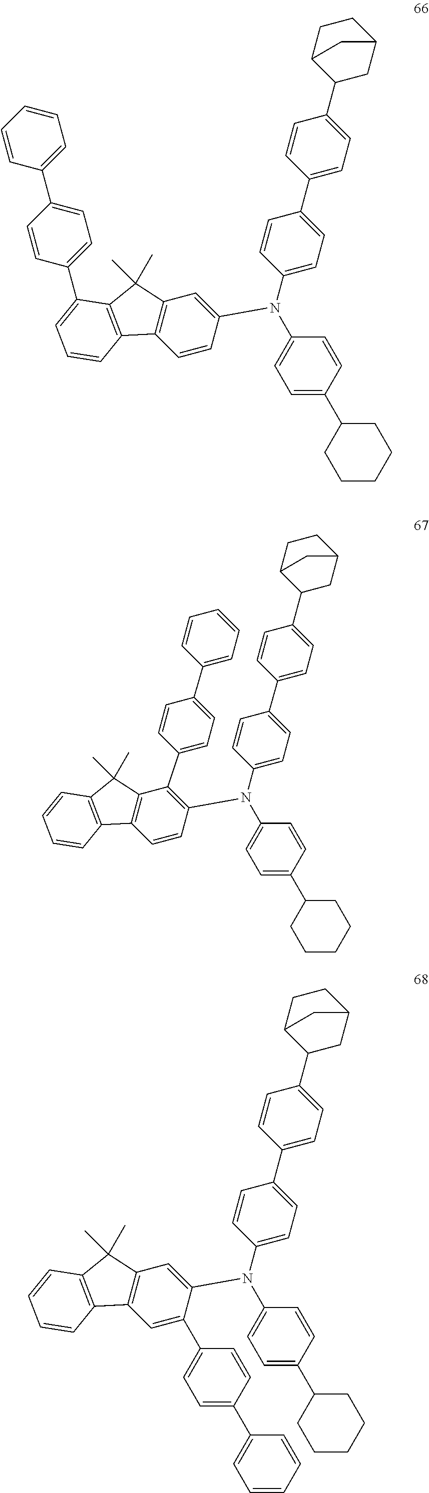

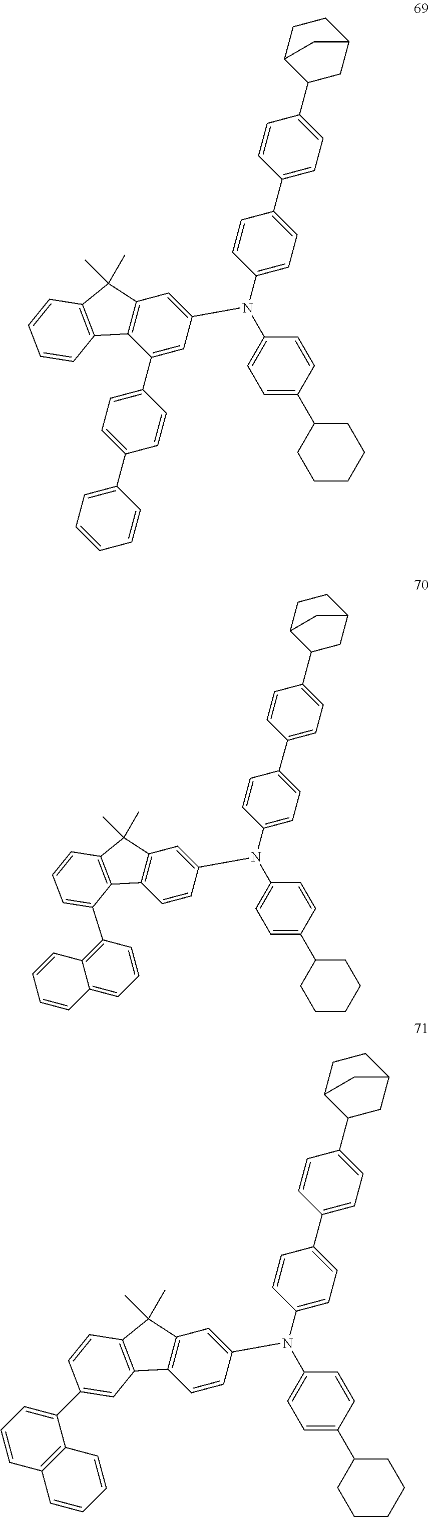

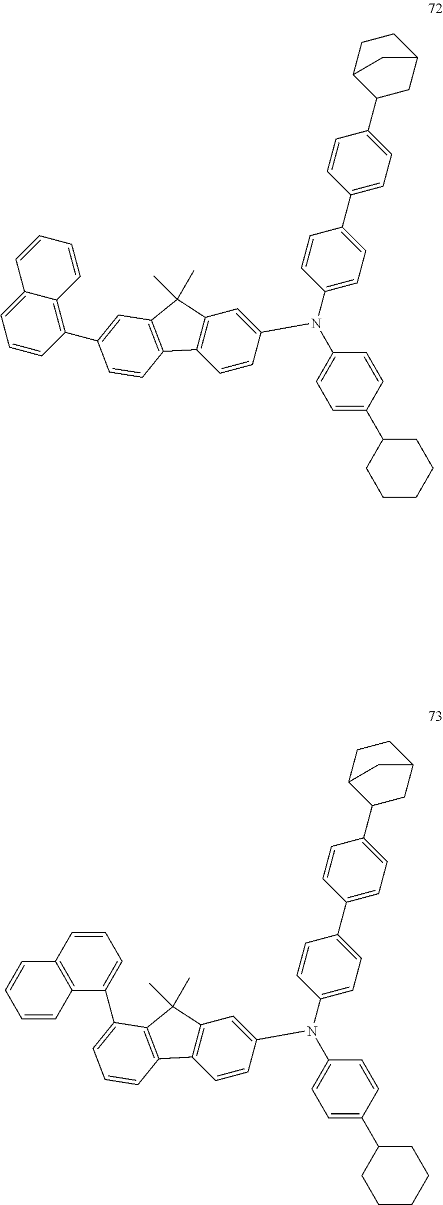 [SMILES: CC1(C)c2cc(-c3cccc4ccccc34)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3cccc4ccccc34)c21]

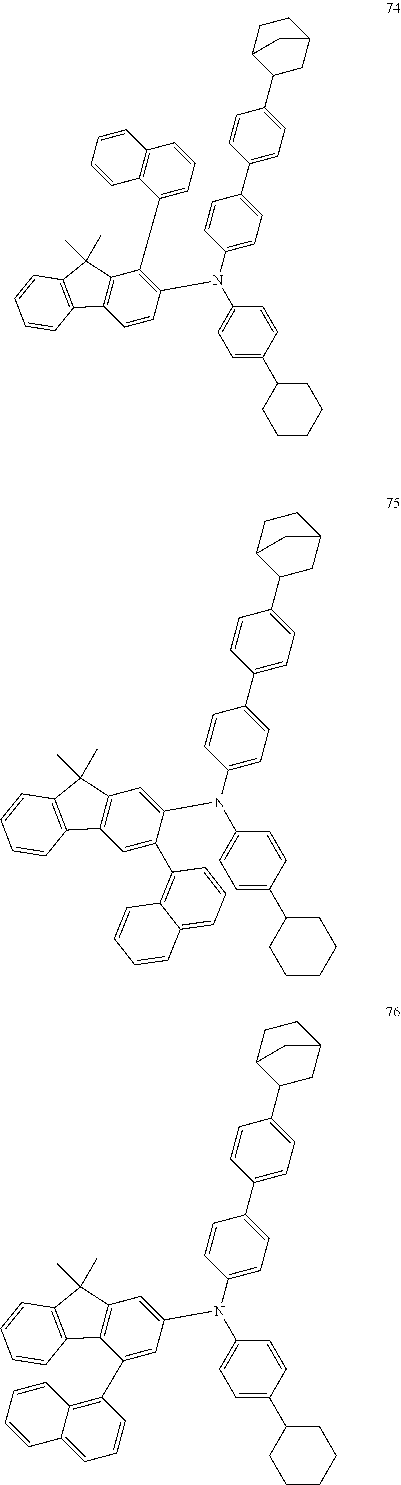 [SMILES: CC1(C)c2ccccc2-c2ccc(N(c3ccc(-c4ccc(C5CC6CCC5C6)cc4)cc3)c3ccc(C4CCCCC4)cc3)c(-c3cccc4ccccc34)c21 ; CC1(C)c2ccccc2-c2cc(-c3cccc4ccccc34)c(N(c3ccc(-c4ccc(C5CC6CCC5C6)cc4)cc3)c3ccc(C4CCCCC4)cc3)cc21 ; CC1(C)c2ccccc2-c2c(-c3cccc4ccccc34)cc(N(c3ccc(-c4ccc(C5CC6CCC5C6)cc4)cc3)c3ccc(C4CCCCC4)cc3)cc21]

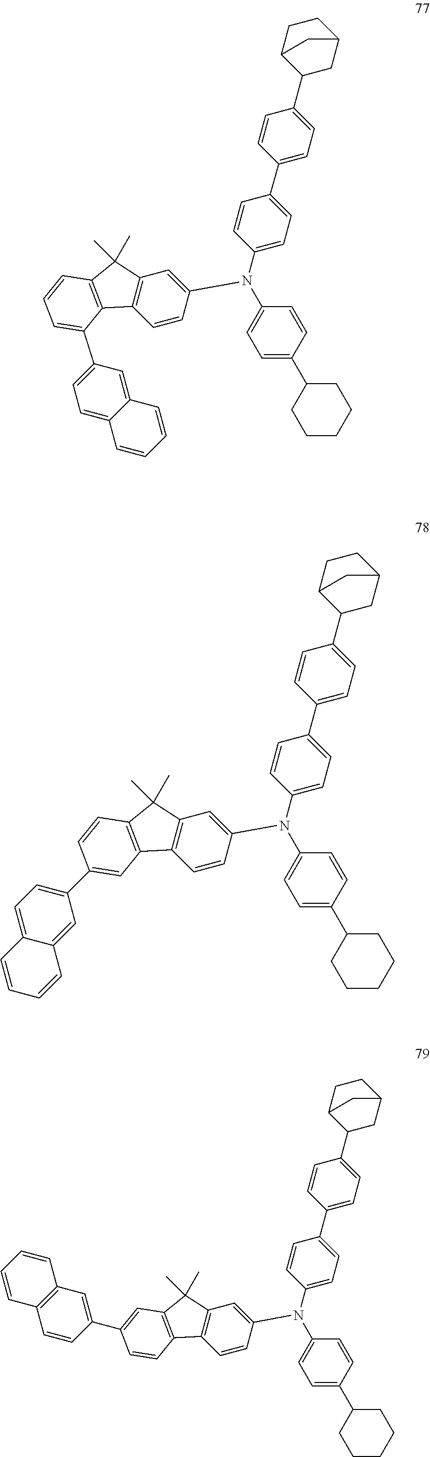 [SMILES: CC1(C)c2cc(N(c3ccc(-c4ccc(C5CC6CCC5C6)cc4)cc3)c3ccc(C4CCCCC4)cc3)ccc2-c2c(-c3ccc4ccccc4c3)cccc21 ; CC1(C)c2ccc(-c3ccc4ccccc4c3)cc2-c2ccc(N(c3ccc(-c4ccc(C5CC6CCC5C6)cc4)cc3)c3ccc(C4CCCCC4)cc3)cc21 ; CC1(C)c2cc(-c3ccc4ccccc4c3)ccc2-c2ccc(N(c3ccc(-c4ccc(C5CC6CCC5C6)cc4)cc3)c3ccc(C4CCCCC4)cc3)cc21]

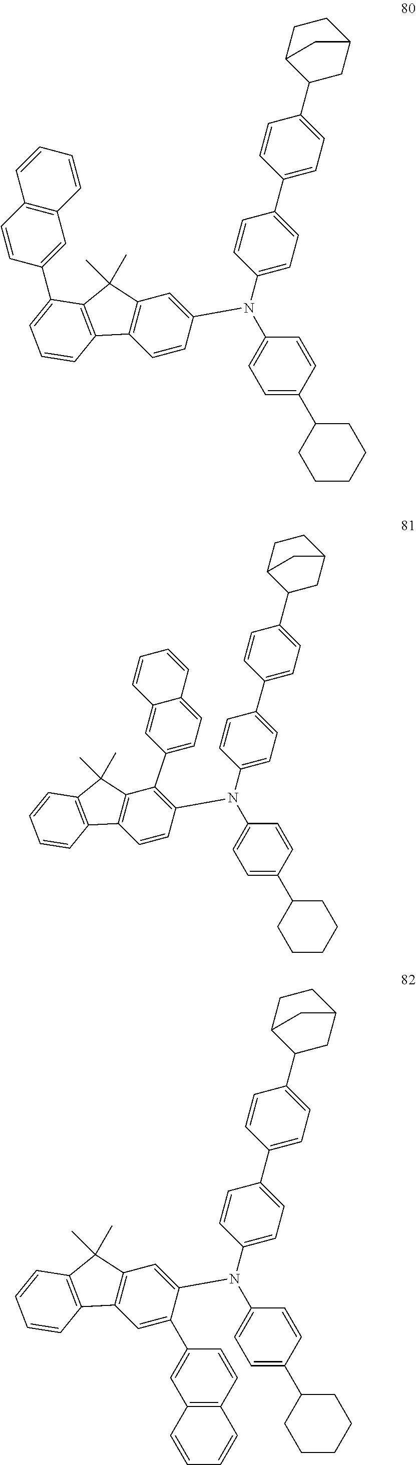

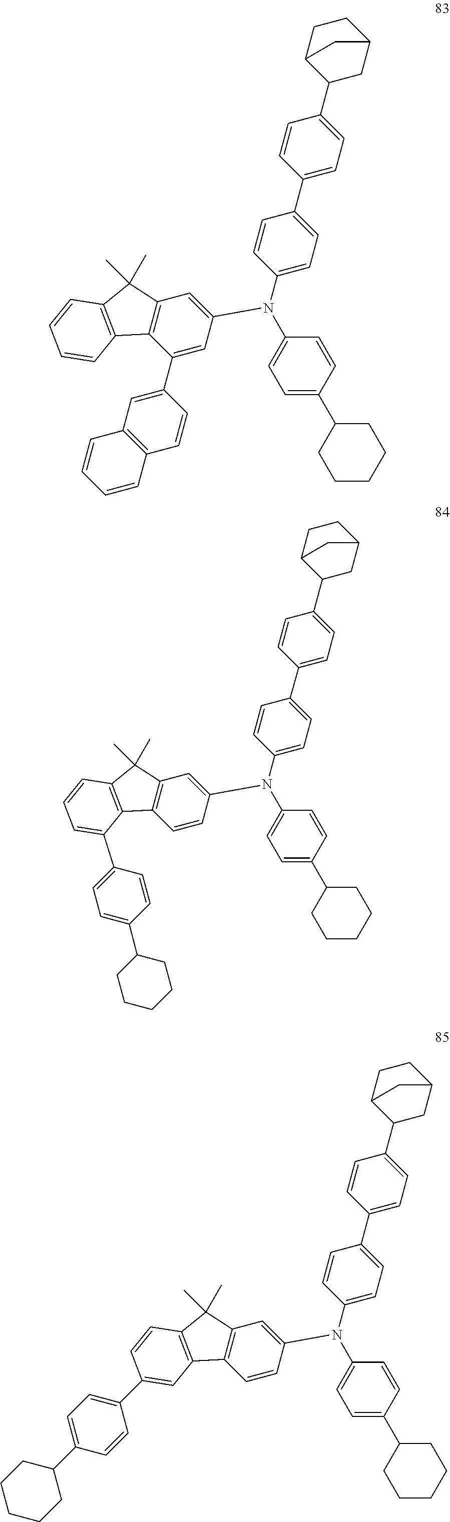

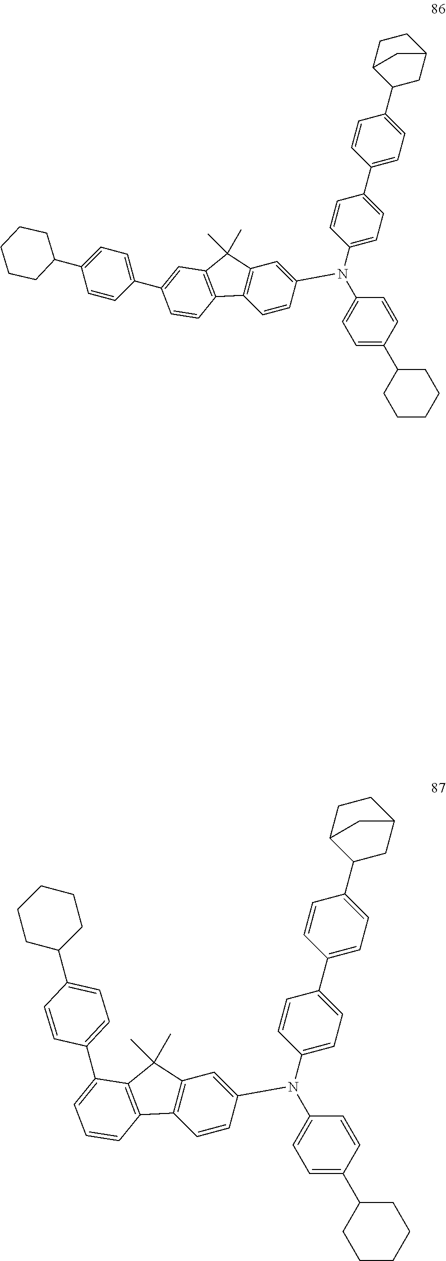 [SMILES: CC1(C)c2cc(-c3ccc(C4CCCCC4)cc3)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3ccc(C4CCCCC4)cc3)c21]

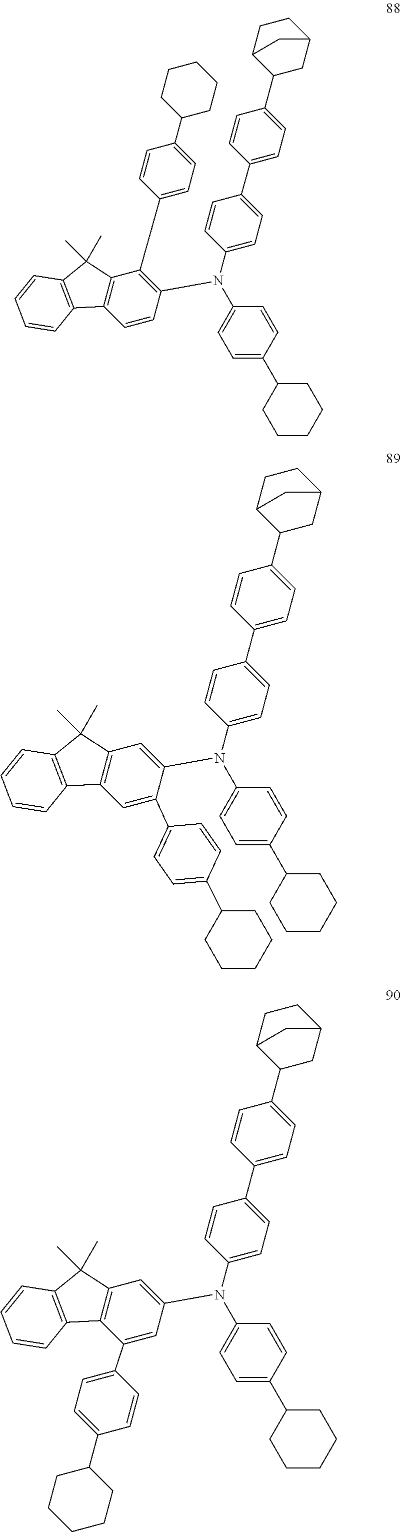

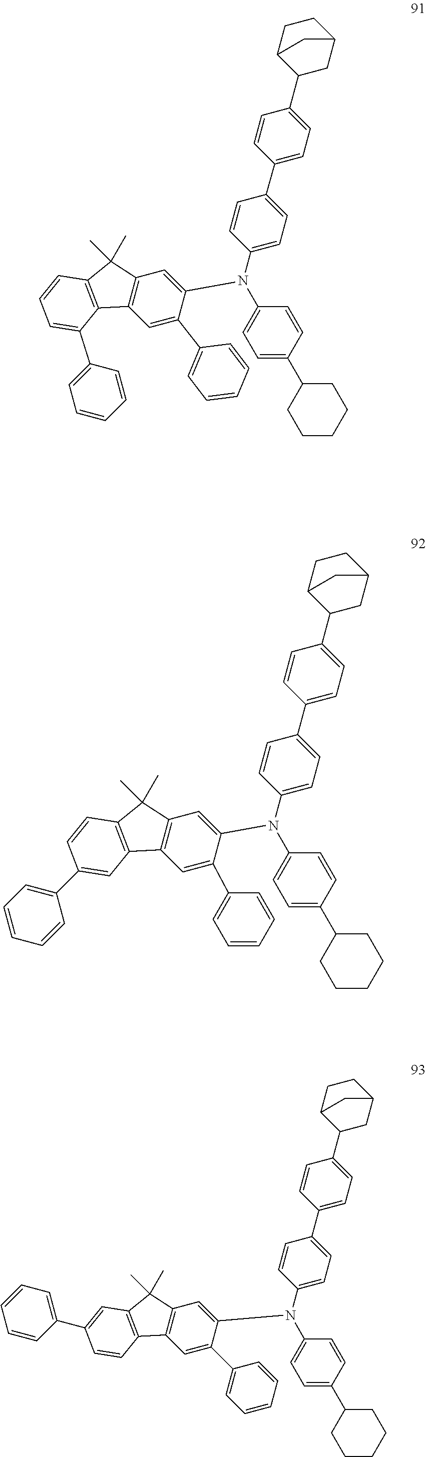 [SMILES: CC1(C)c2cc(N(c3ccc(-c4ccc(C5CC6CCC5C6)cc4)cc3)c3ccc(C4CCCCC4)cc3)c(-c3ccccc3)cc2-c2c(-c3ccccc3)cccc21 ; CC1(C)c2ccc(-c3ccccc3)cc2-c2cc(-c3ccccc3)c(N(c3ccc(-c4ccc(C5CC6CCC5C6)cc4)cc3)c3ccc(C4CCCCC4)cc3)cc21 ; CC1(C)c2cc(-c3ccccc3)ccc2-c2cc(-c3ccccc3)c(N(c3ccc(-c4ccc(C5CC6CCC5C6)cc4)cc3)c3ccc(C4CCCCC4)cc3)cc21]

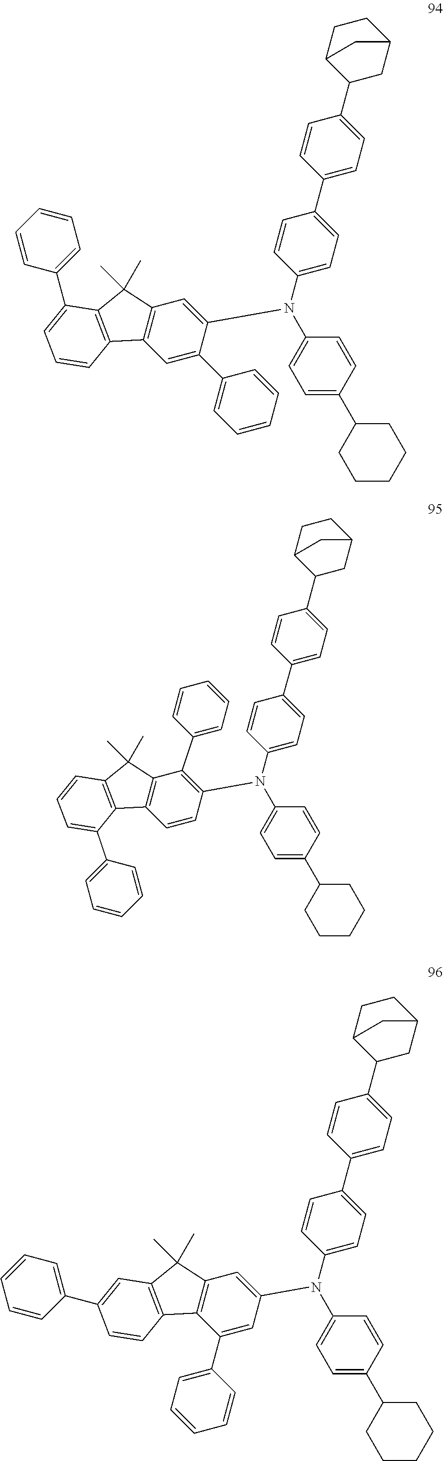

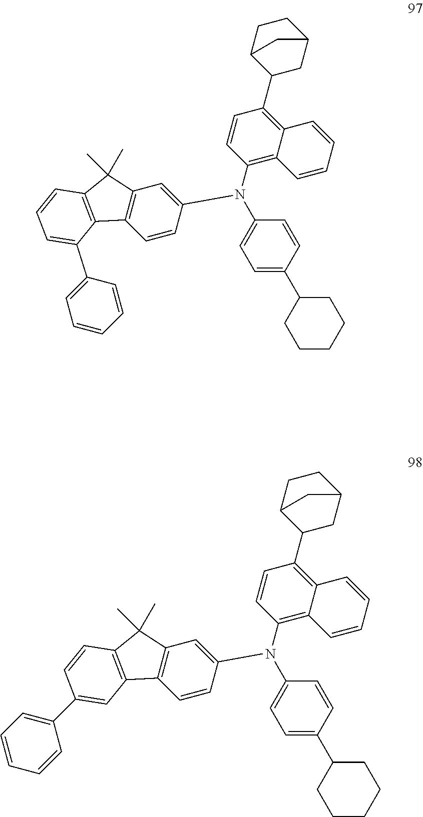 [SMILES: CC1(C)c2cc(N(c3ccc(C4CCCCC4)cc3)c3ccc(C4CC5CCC4C5)c4ccccc34)ccc2-c2c(-c3ccccc3)cccc21 ; CC1(C)c2ccc(-c3ccccc3)cc2-c2ccc(N(c3ccc(C4CCCCC4)cc3)c3ccc(C4CC5CCC4C5)c4ccccc34)cc21]

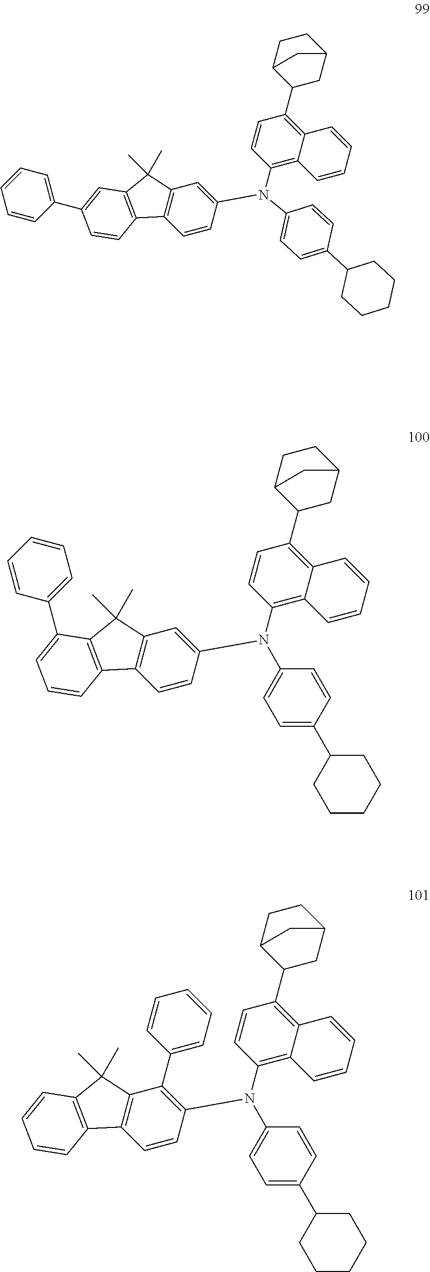 [SMILES: CC1(C)c2cc(-c3ccccc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccccc3)c21 ; CC1(C)c2ccccc2-c2ccc(N(c3ccc(C4CCCCC4)cc3)c3ccc(C4CC5CCC4C5)c4ccccc34)c(-c3ccccc3)c21]

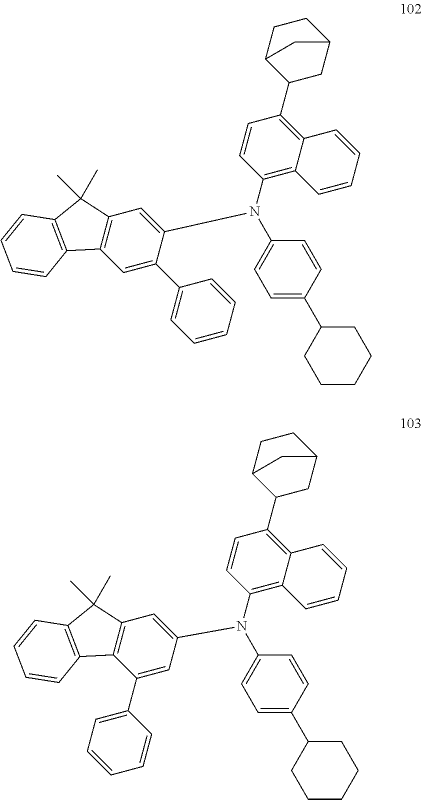 [SMILES: CC1(C)c2ccccc2-c2cc(-c3ccccc3)c(N(c3ccc(C4CCCCC4)cc3)c3ccc(C4CC5CCC4C5)c4ccccc34)cc21 ; CC1(C)c2ccccc2-c2c(-c3ccccc3)cc(N(c3ccc(C4CCCCC4)cc3)c3ccc(C4CC5CCC4C5)c4ccccc34)cc21]

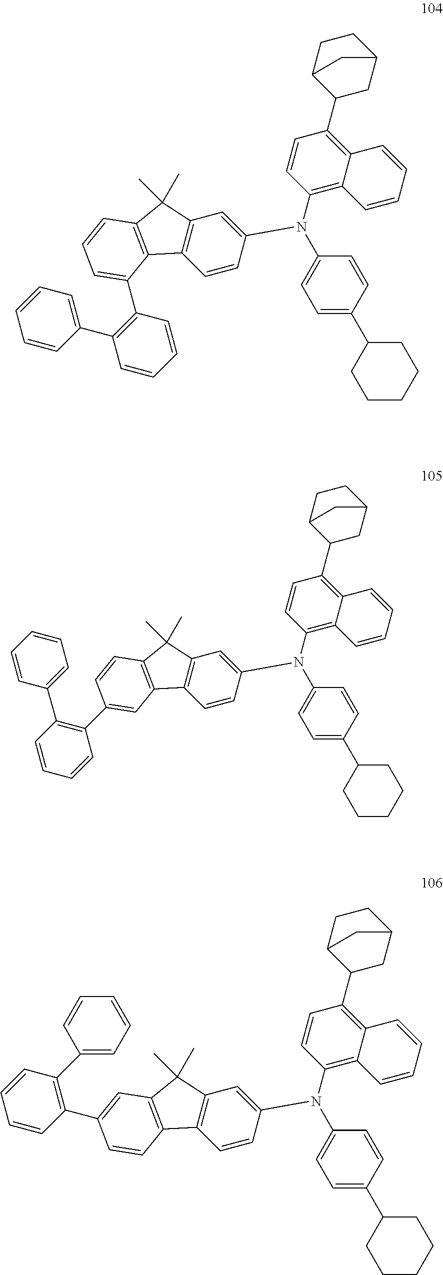 [SMILES: CC1(C)c2cc(N(c3ccc(C4CCCCC4)cc3)c3ccc(C4CC5CCC4C5)c4ccccc34)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4CC5CCC4C5)c4ccccc34)cc21 ; CC1(C)c2cc(-c3ccccc3-c3ccccc3)ccc2-c2ccc(N(c3ccc(C4CCCCC4)cc3)c3ccc(C4CC5CCC4C5)c4ccccc34)cc21]

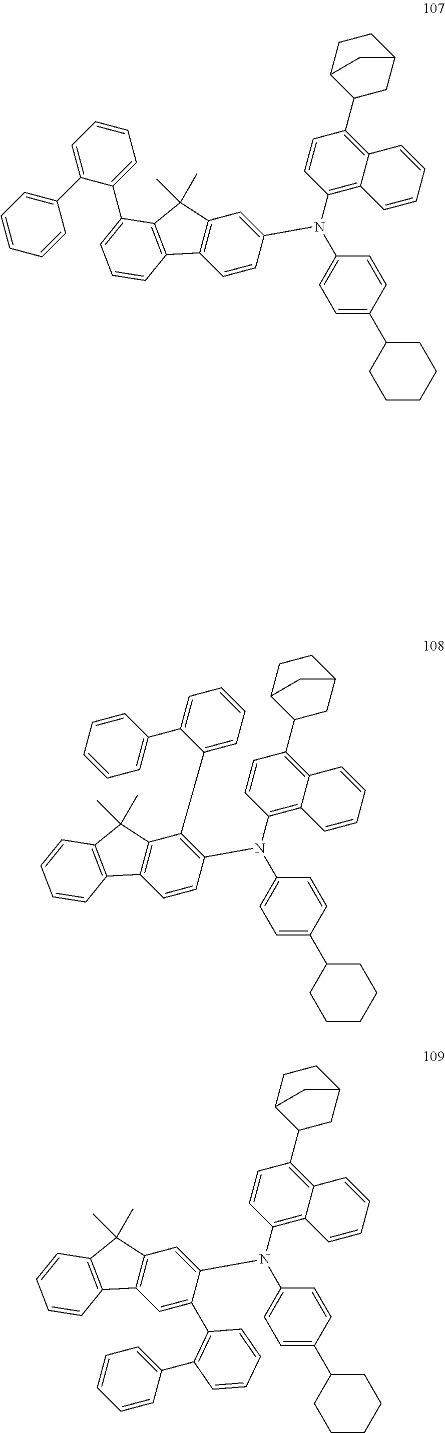

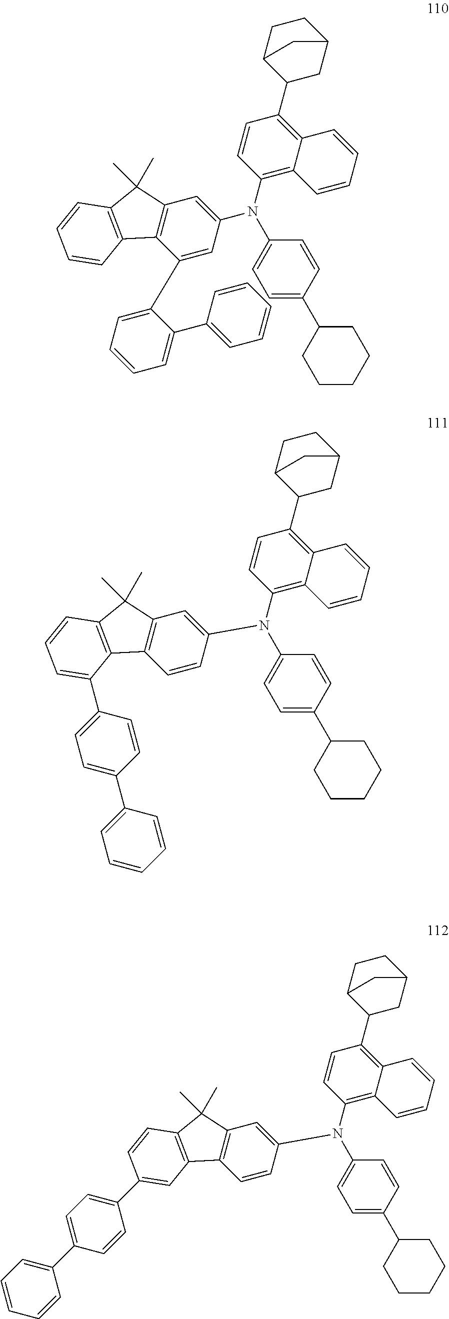 [SMILES: CC1(C)c2ccccc2-c2c(-c3ccccc3-c3ccccc3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4CC5CCC4C5)c4ccccc34)cc21]

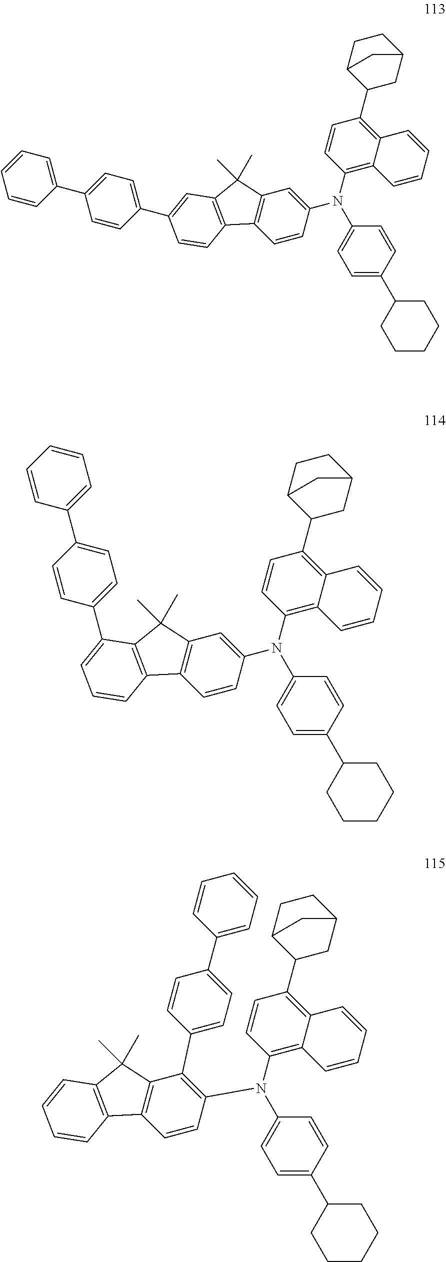 [SMILES: CC1(C)c2cc(-c3ccc(-c4ccccc4)cc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc(-c4ccccc4)cc3)c21 ; CC1(C)c2ccccc2-c2ccc(N(c3ccc(C4CCCCC4)cc3)c3ccc(C4CC5CCC4C5)c4ccccc34)c(-c3ccc(-c4ccccc4)cc3)c21]

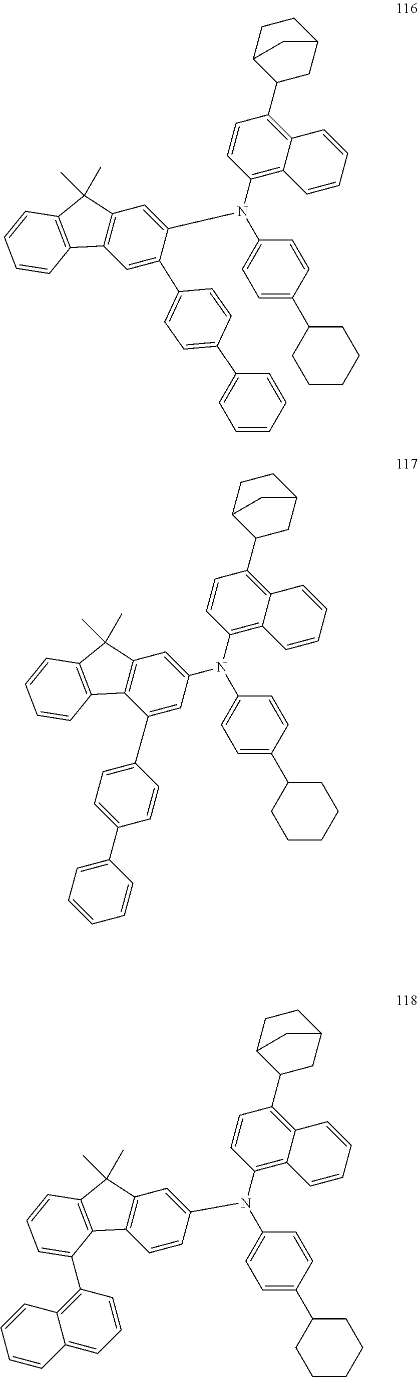

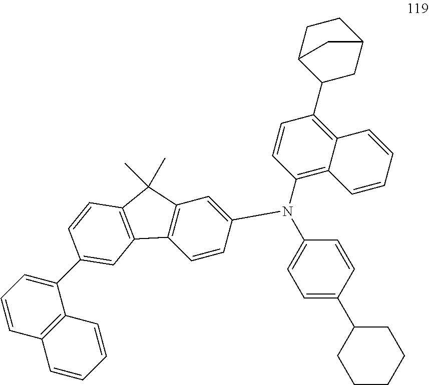 [SMILES: CC1(C)c2ccc(-c3cccc4ccccc34)cc2-c2ccc(N(c3ccc(C4CCCCC4)cc3)c3ccc(C4CC5CCC4C5)c4ccccc34)cc21]

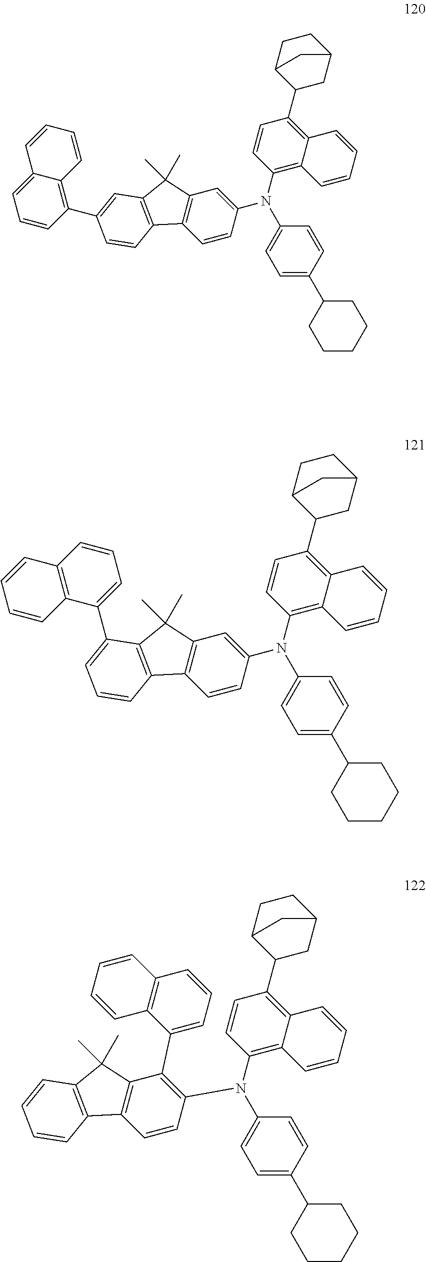 [SMILES: CC1(C)c2cc(-c3cccc4ccccc34)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3cccc4ccccc34)c21 ; CC1(C)c2ccccc2-c2ccc(N(c3ccc(C4CCCCC4)cc3)c3ccc(C4CC5CCC4C5)c4ccccc34)c(-c3cccc4ccccc34)c21]

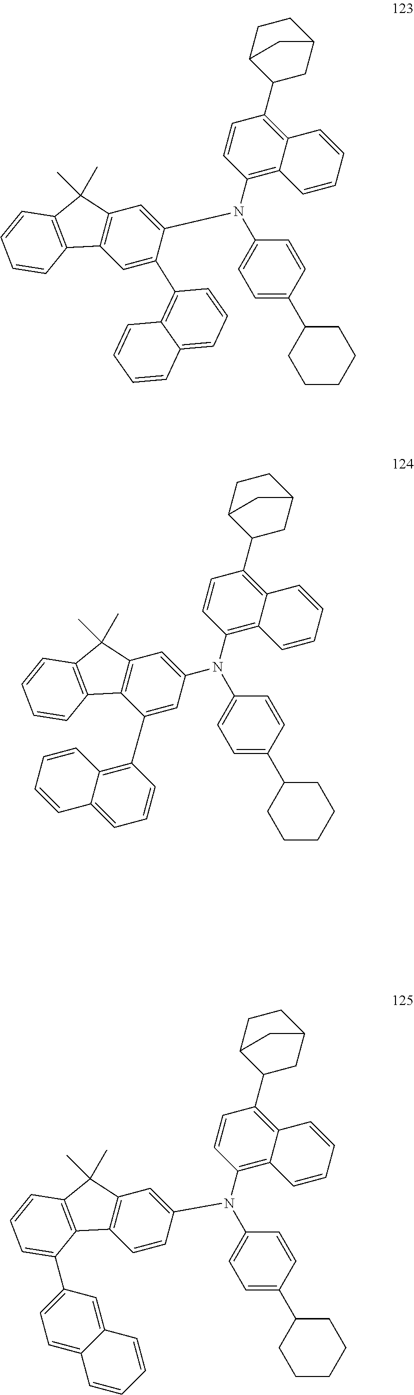 [SMILES: CC1(C)c2ccccc2-c2cc(-c3cccc4ccccc34)c(N(c3ccc(C4CCCCC4)cc3)c3ccc(C4CC5CCC4C5)c4ccccc34)cc21 ; CC1(C)c2ccccc2-c2c(-c3cccc4ccccc34)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc4ccccc4c3)cccc21]

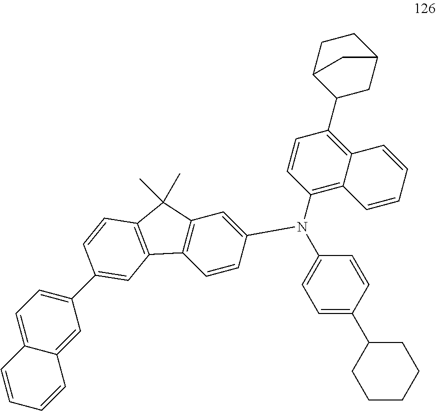 [SMILES: CC1(C)c2ccc(-c3ccc4ccccc4c3)cc2-c2ccc(N(c3ccc(C4CCCCC4)cc3)c3ccc(C4CC5CCC4C5)c4ccccc34)cc21]

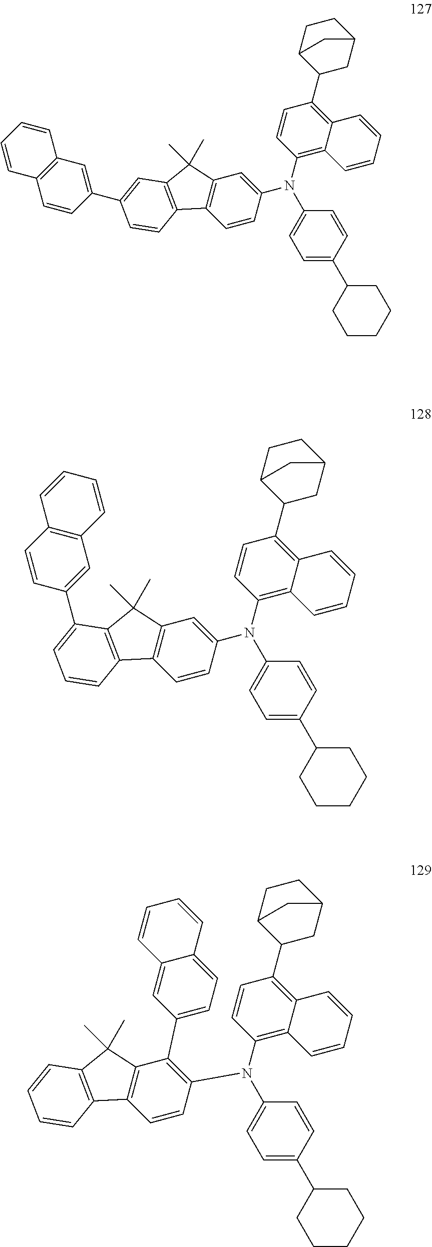 [SMILES: CC1(C)c2cc(-c3ccc4ccccc4c3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc4ccccc4c3)c21 ; CC1(C)c2ccccc2-c2ccc(N(c3ccc(C4CCCCC4)cc3)c3ccc(C4CC5CCC4C5)c4ccccc34)c(-c3ccc4ccccc4c3)c21]

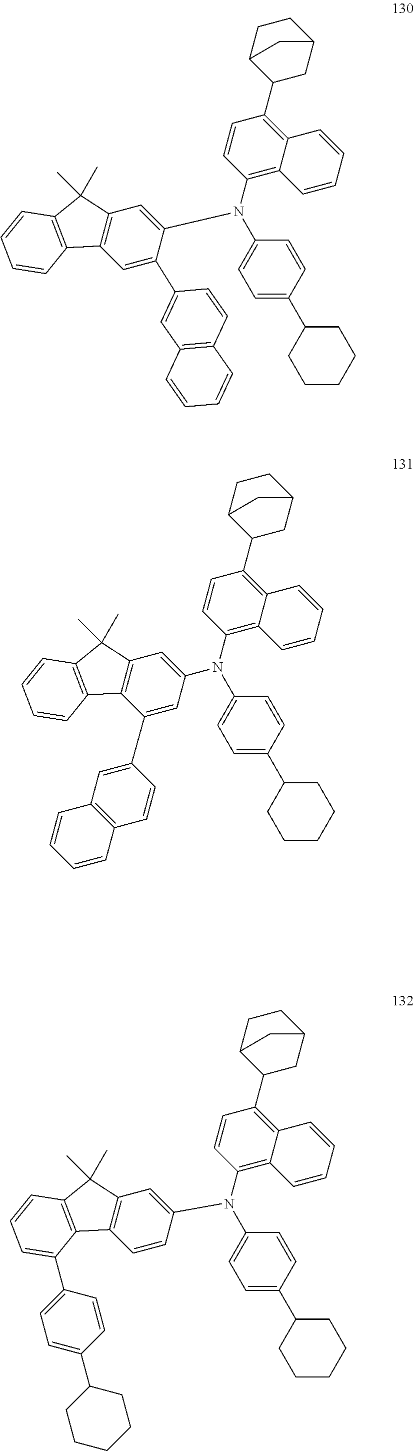 [SMILES: CC1(C)c2ccccc2-c2cc(-c3ccc4ccccc4c3)c(N(c3ccc(C4CCCCC4)cc3)c3ccc(C4CC5CCC4C5)c4ccccc34)cc21 ; CC1(C)c2ccccc2-c2c(-c3ccc4ccccc4c3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc(C4CCCCC4)cc3)cccc21]

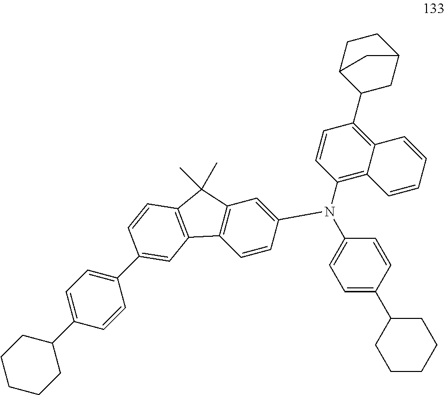 [SMILES: CC1(C)c2ccc(-c3ccc(C4CCCCC4)cc3)cc2-c2ccc(N(c3ccc(C4CCCCC4)cc3)c3ccc(C4CC5CCC4C5)c4ccccc34)cc21]

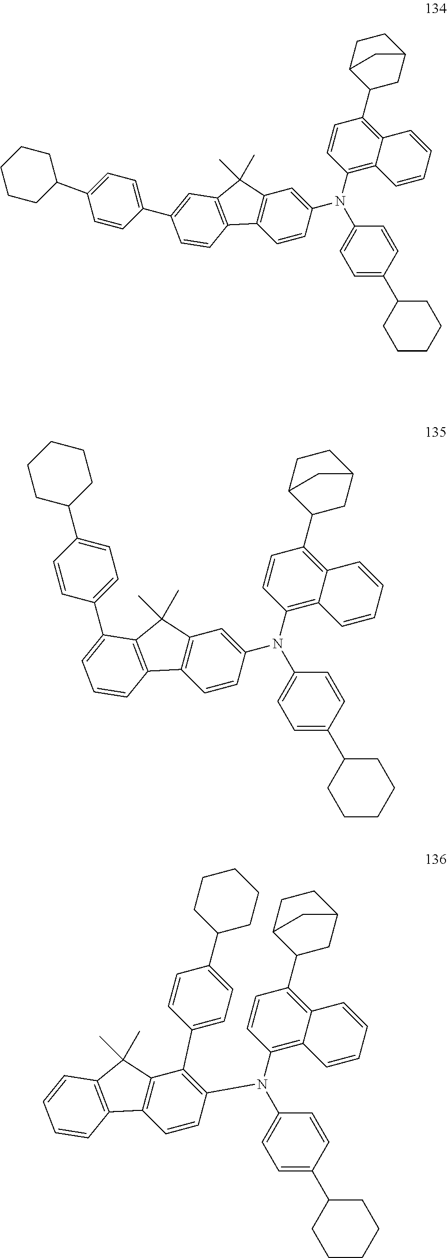 [SMILES: CC1(C)c2cc(-c3ccc(C4CCCCC4)cc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc(C4CCCCC4)cc3)c21 ; CC1(C)c2ccccc2-c2ccc(N(c3ccc(C4CCCCC4)cc3)c3ccc(C4CC5CCC4C5)c4ccccc34)c(-c3ccc(C4CCCCC4)cc3)c21]

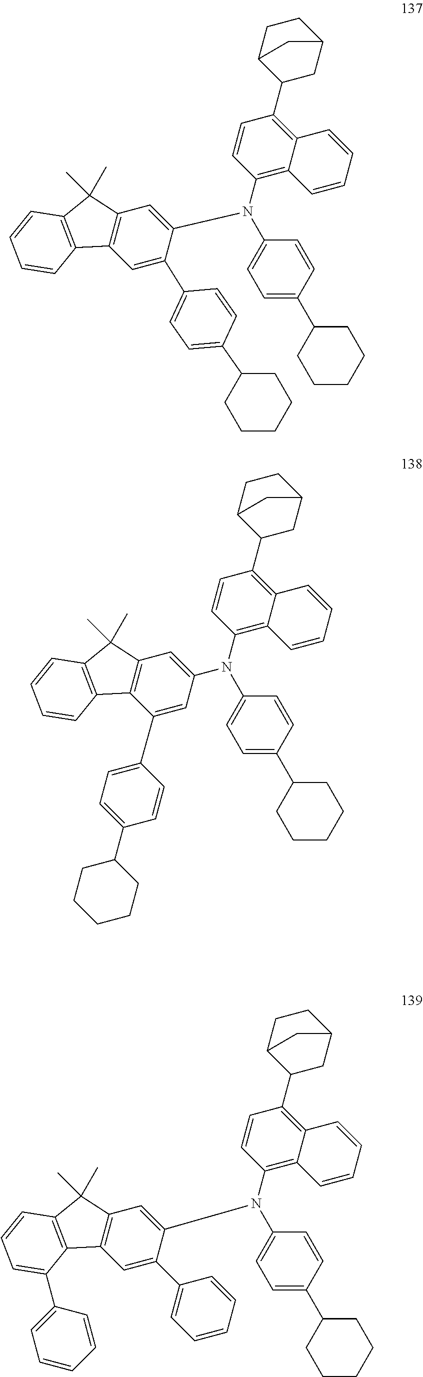

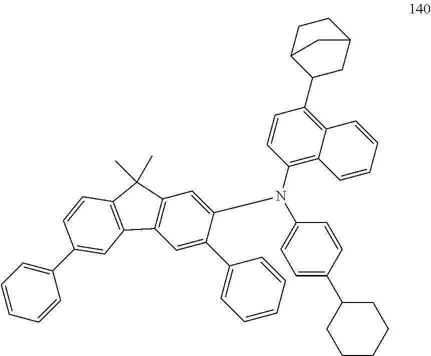 [SMILES: CC1(C)c2ccc(-c3ccccc3)cc2-c2cc(-c3ccccc3)c(N(c3ccc(C4CCCCC4)cc3)c3ccc(C4CC5CCC4C5)c4ccccc34)cc21]

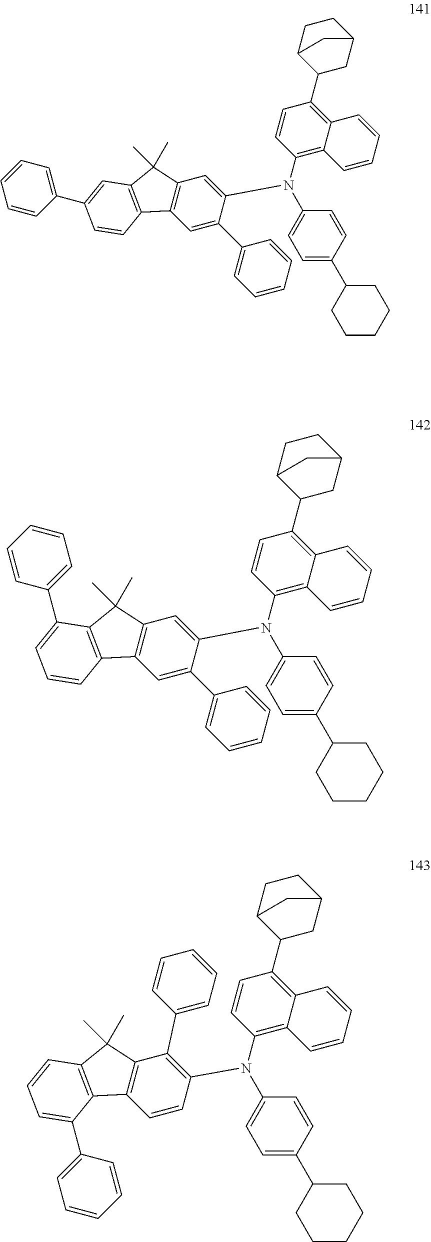 [SMILES: CC1(C)c2cc(-c3ccccc3)ccc2-c2cc(-c3ccccc3)c(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4CC5CCC4C5)c4ccccc34)c(-c3ccccc3)c21]

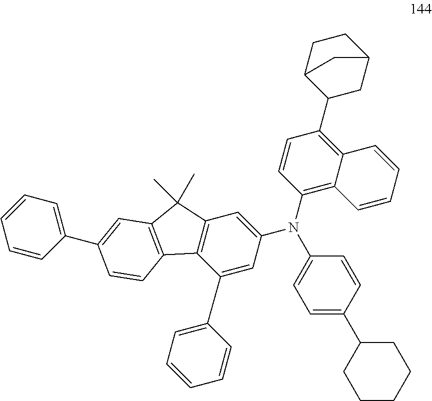 [SMILES: CC1(C)c2cc(-c3ccccc3)ccc2-c2c(-c3ccccc3)cc(N(c3ccc(C4CCCCC4)cc3)c3ccc(C4CC5CCC4C5)c4ccccc34)cc21]

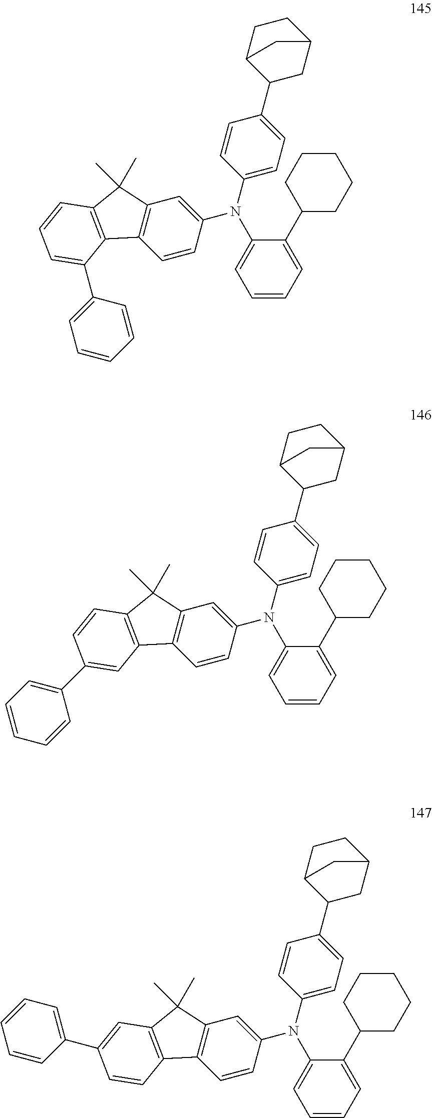 [SMILES: CC1(C)c2cc(N(c3ccc(C4CC5CCC4C5)cc3)c3ccccc3C3CCCCC3)ccc2-c2c(-c3ccccc3)cccc21 ; CC1(C)c2ccc(-c3ccccc3)cc2-c2ccc(N(c3ccc(C4CC5CCC4C5)cc3)c3ccccc3C3CCCCC3)cc21 ; CC1(C)c2cc(-c3ccccc3)ccc2-c2ccc(N(c3ccc(C4CC5CCC4C5)cc3)c3ccccc3C3CCCCC3)cc21]

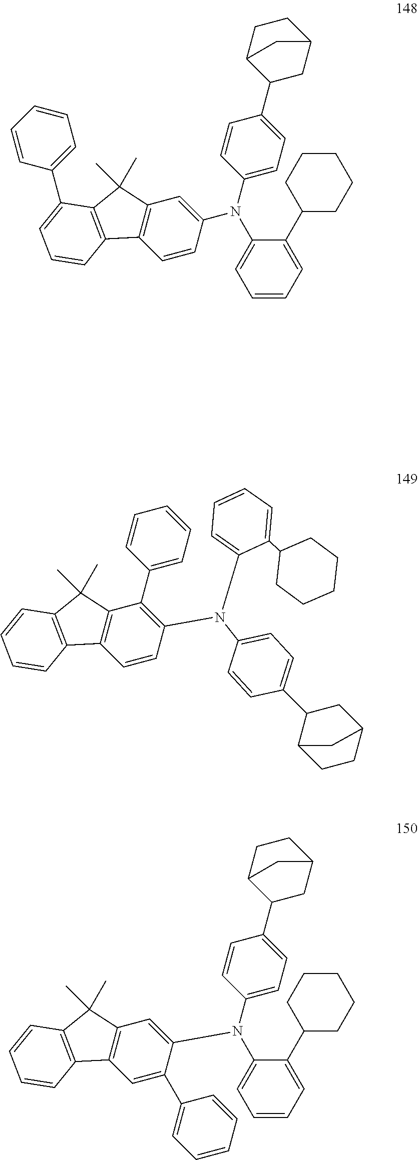 [SMILES: CC1(C)c2cc(N(c3ccc(C4CC5CCC4C5)cc3)c3ccccc3C3CCCCC3)ccc2-c2cccc(-c3ccccc3)c21 ; CC1(C)c2ccccc2-c2ccc(N(c3ccc(C4CC5CCC4C5)cc3)c3ccccc3C3CCCCC3)c(-c3ccccc3)c21 ; CC1(C)c2ccccc2-c2cc(-c3ccccc3)c(N(c3ccc(C4CC5CCC4C5)cc3)c3ccccc3C3CCCCC3)cc21]

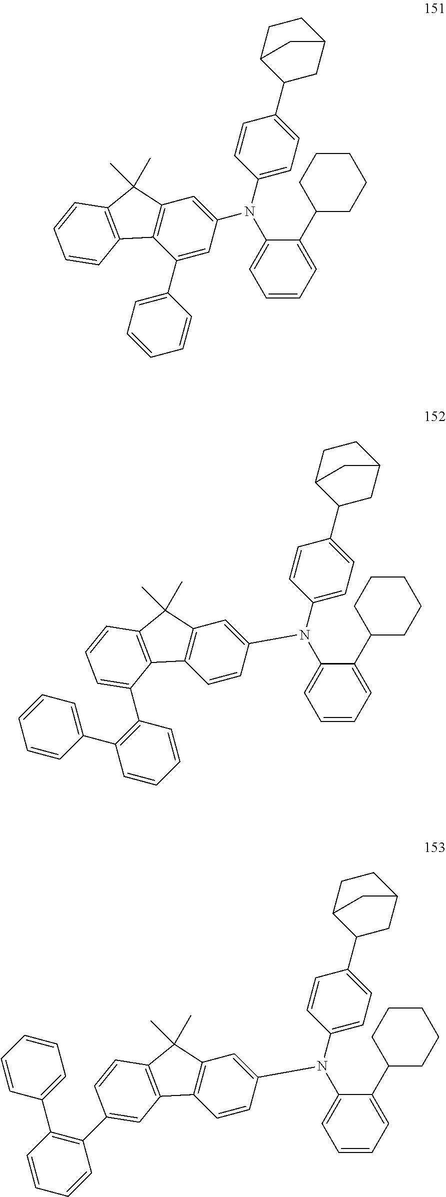 [SMILES: CC1(C)c2ccccc2-c2c(-c3ccccc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3ccccc3C3CCCCC3)cc21]

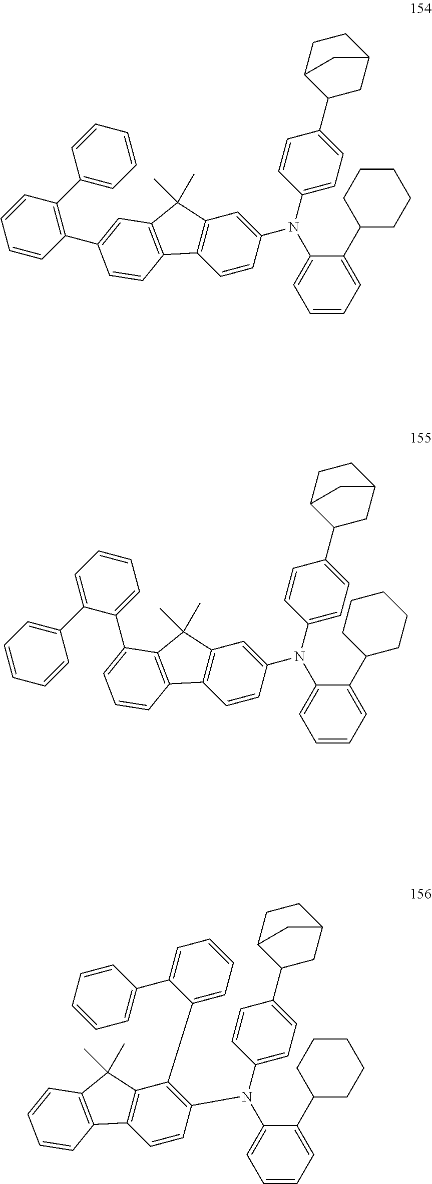 [SMILES: CC1(C)c2cc(-c3ccccc3-c3ccccc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccccc3-c3ccccc3)c21 ; CC1(C)c2ccccc2-c2ccc(N(c3ccc(C4CC5CCC4C5)cc3)c3ccccc3C3CCCCC3)c(-c3ccccc3-c3ccccc3)c21]

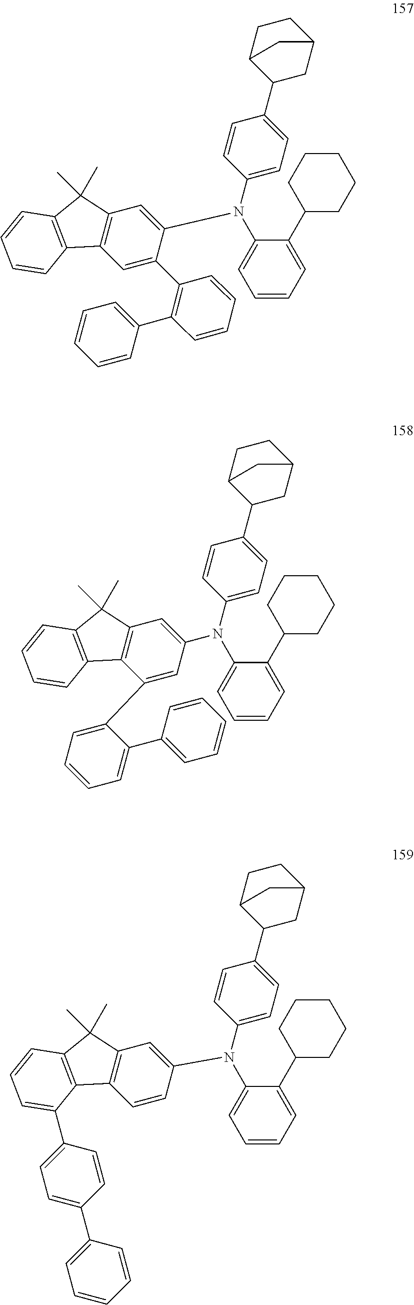 [SMILES: CC1(C)c2ccccc2-c2cc(-c3ccccc3-c3ccccc3)c(N(c3ccc(C4CC5CCC4C5)cc3)c3ccccc3C3CCCCC3)cc21 ; CC1(C)c2ccccc2-c2c(-c3ccccc3-c3ccccc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc(-c4ccccc4)cc3)cccc21]

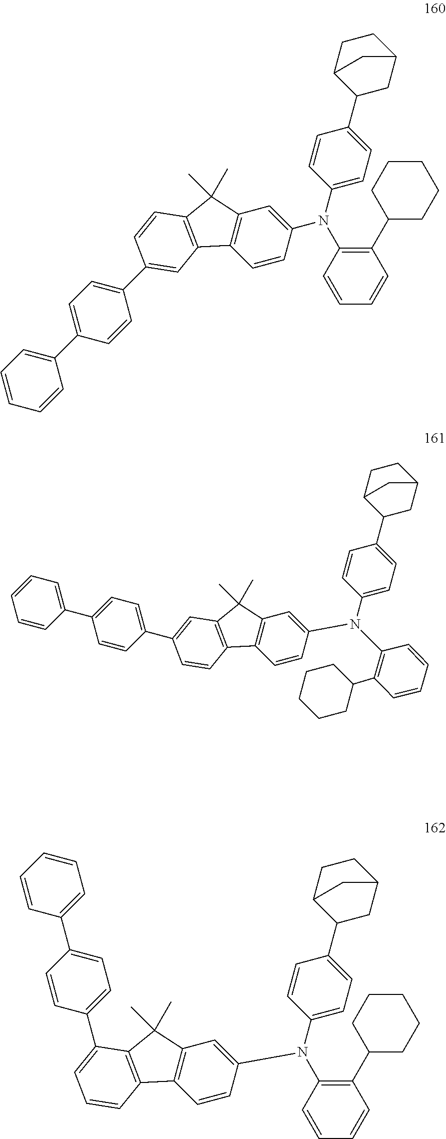 [SMILES: CC1(C)c2ccc(-c3ccc(-c4ccccc4)cc3)cc2-c2ccc(N(c3ccc(C4CC5CCC4C5)cc3)c3ccccc3C3CCCCC3)cc21 ; CC1(C)c2cc(-c3ccc(-c4ccccc4)cc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc(-c4ccccc4)cc3)c21]

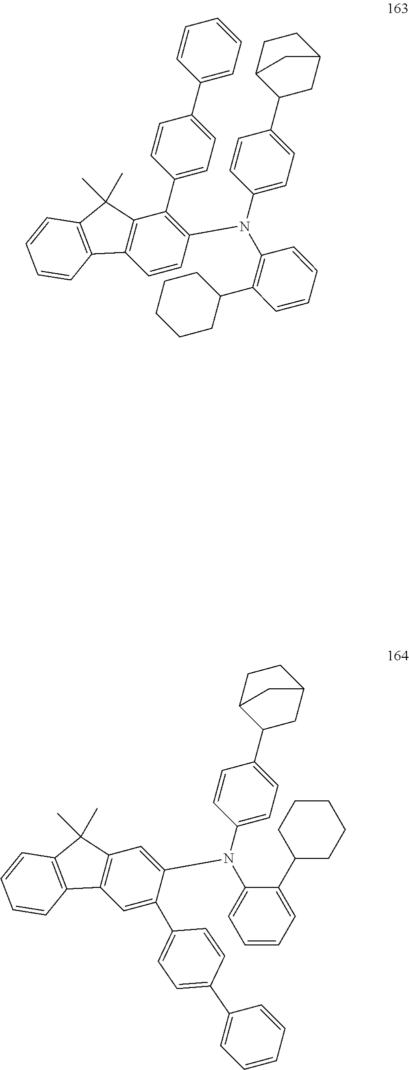 [SMILES: CC1(C)c2ccccc2-c2ccc(N(c3ccc(C4CC5CCC4C5)cc3)c3ccccc3C3CCCCC3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3ccccc3C3CCCCC3)cc21]

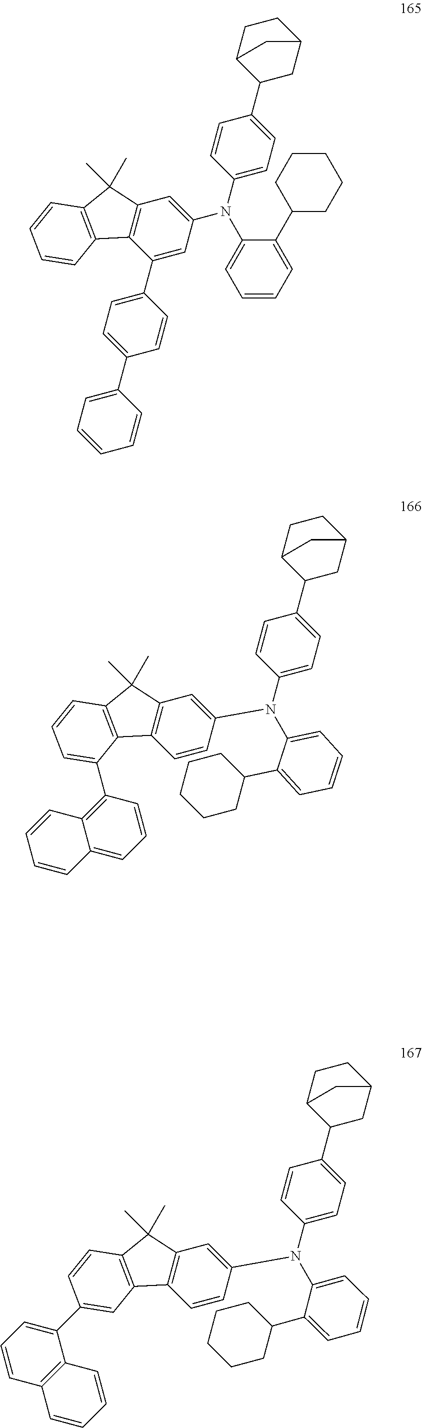 [SMILES: CC1(C)c2ccccc2-c2c(-c3ccc(-c4ccccc4)cc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccccc3C3CCCCC3)cc21]

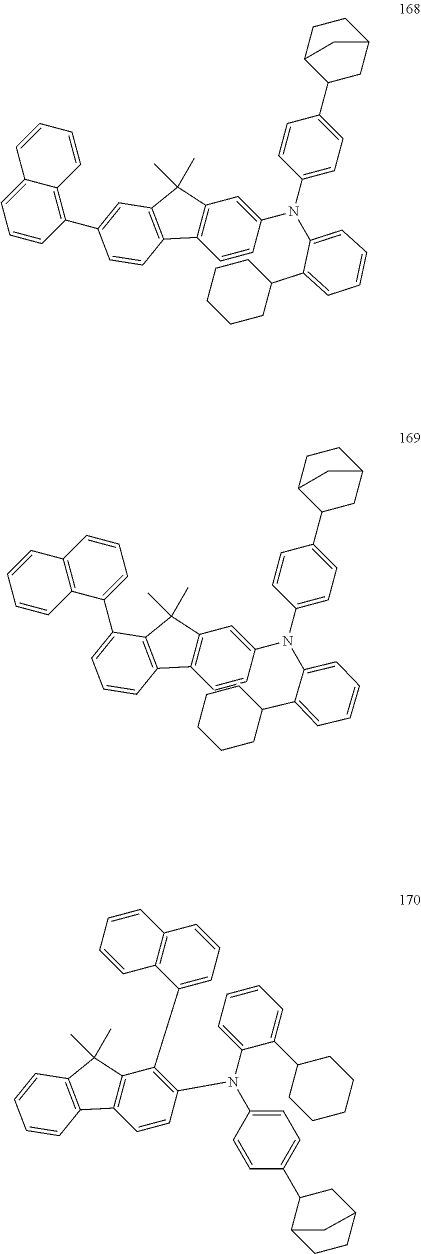 [SMILES: CC1(C)c2cc(-c3cccc4ccccc34)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3cccc4ccccc34)c21 ; CC1(C)c2ccccc2-c2ccc(N(c3ccc(C4CC5CCC4C5)cc3)c3ccccc3C3CCCCC3)c(-c3cccc4ccccc34)c21]

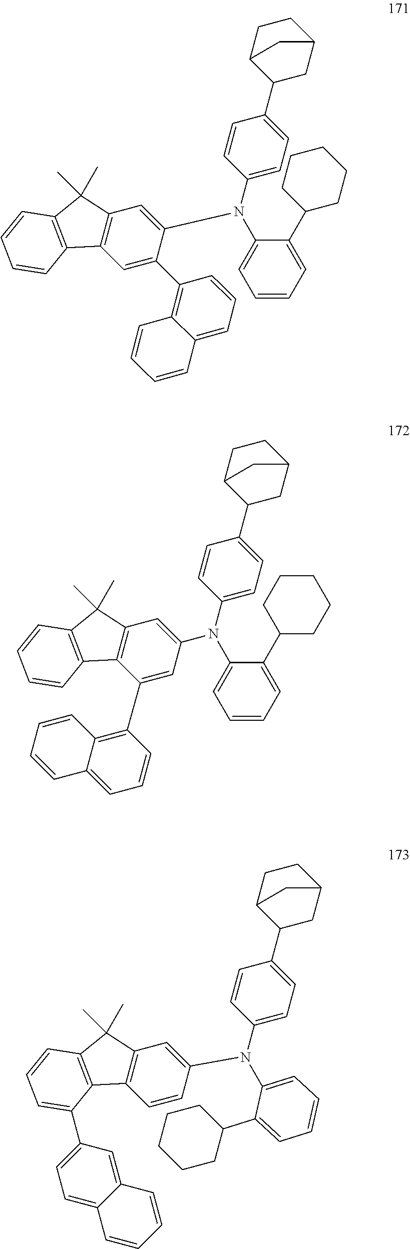 [SMILES: CC1(C)c2ccccc2-c2cc(-c3cccc4ccccc34)c(N(c3ccc(C4CC5CCC4C5)cc3)c3ccccc3C3CCCCC3)cc21 ; CC1(C)c2ccccc2-c2c(-c3cccc4ccccc34)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc4ccccc4c3)cccc21]

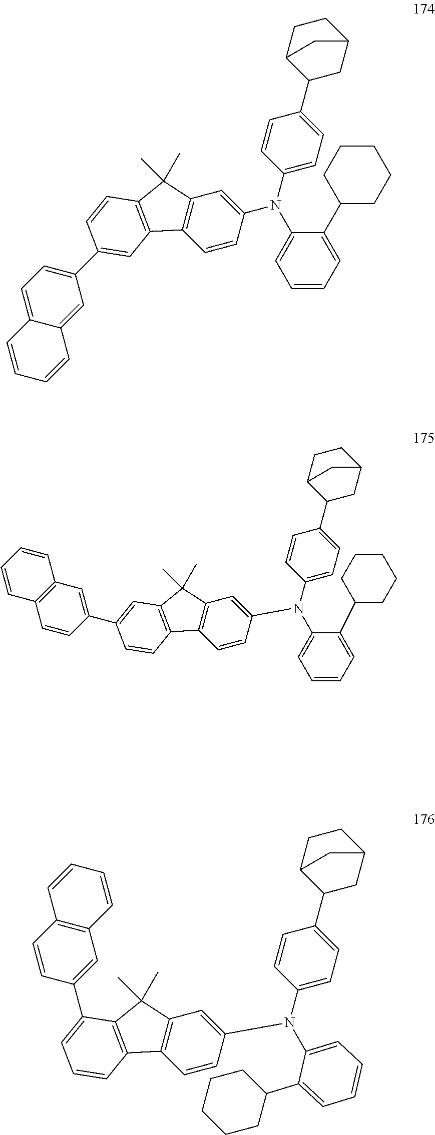 [SMILES: CC1(C)c2ccc(-c3ccc4ccccc4c3)cc2-c2ccc(N(c3ccc(C4CC5CCC4C5)cc3)c3ccccc3C3CCCCC3)cc21 ; CC1(C)c2cc(-c3ccc4ccccc4c3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc4ccccc4c3)c21]

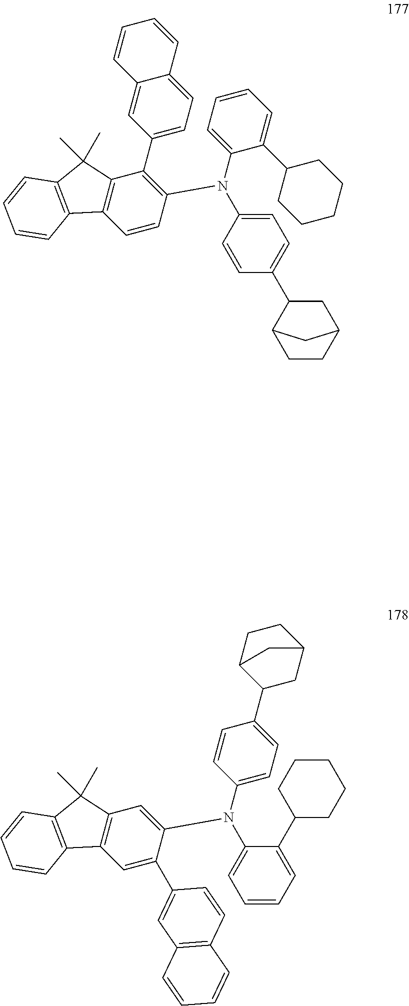 [SMILES: CC1(C)c2ccccc2-c2ccc(N(c3ccc(C4CC5CCC4C5)cc3)c3ccccc3C3CCCCC3)c(-c3ccc4ccccc4c3)c21 ; CC1(C)c2ccccc2-c2cc(-c3ccc4ccccc4c3)c(N(c3ccc(C4CC5CCC4C5)cc3)c3ccccc3C3CCCCC3)cc21]

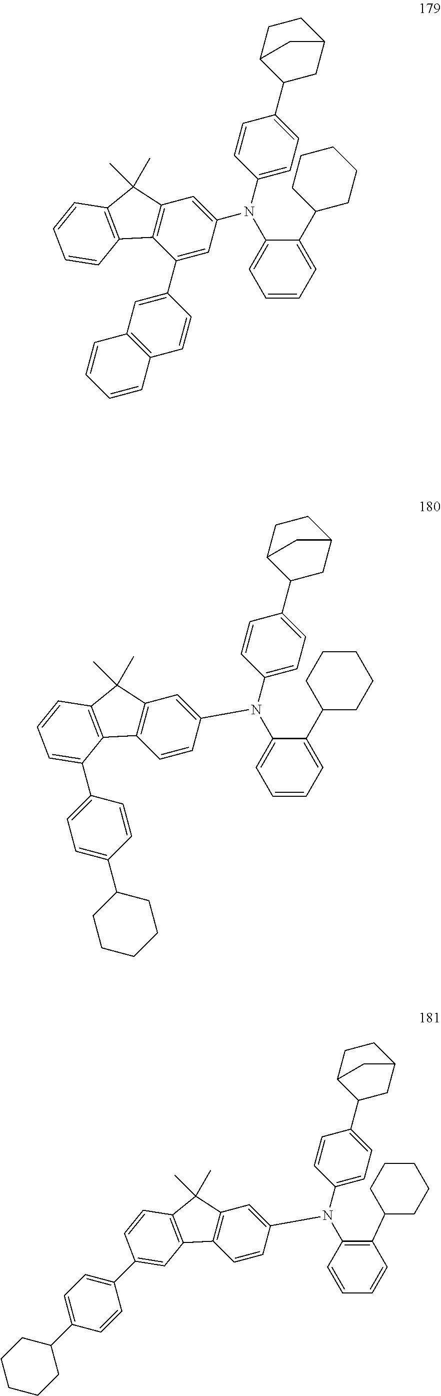 [SMILES: CC1(C)c2ccccc2-c2c(-c3ccc4ccccc4c3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3ccccc3C3CCCCC3)cc21]

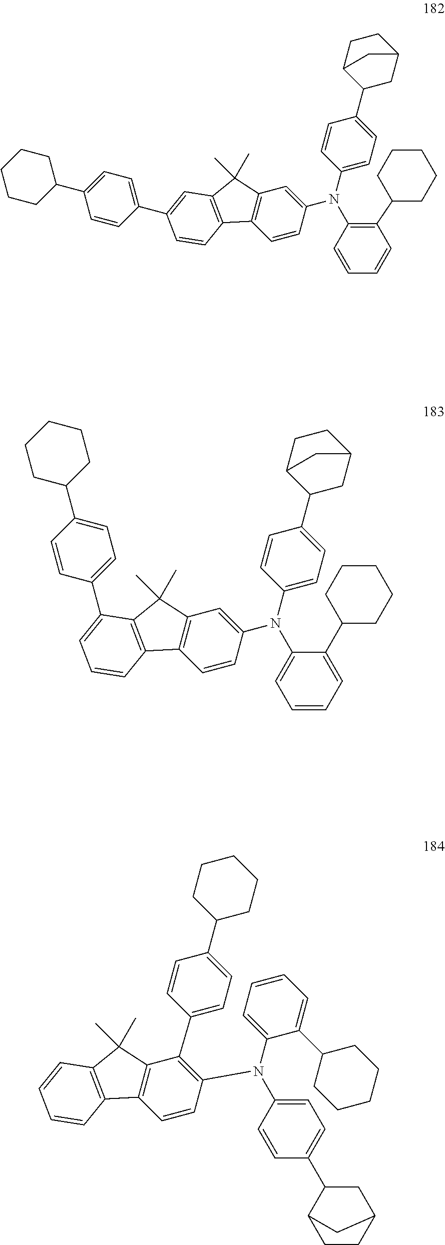 [SMILES: CC1(C)c2cc(-c3ccc(C4CCCCC4)cc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc(C4CCCCC4)cc3)c21 ; CC1(C)c2ccccc2-c2ccc(N(c3ccc(C4CC5CCC4C5)cc3)c3ccccc3C3CCCCC3)c(-c3ccc(C4CCCCC4)cc3)c21]

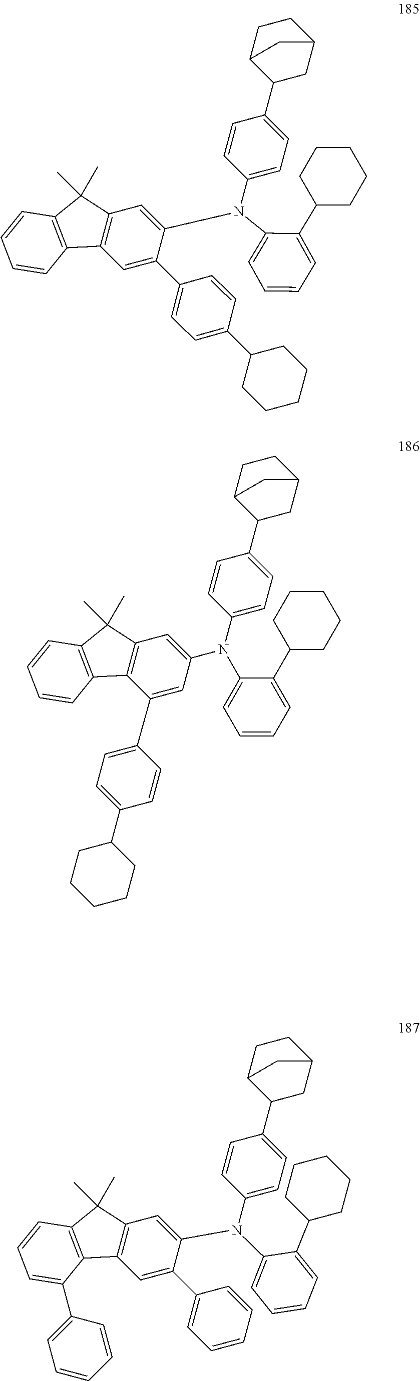

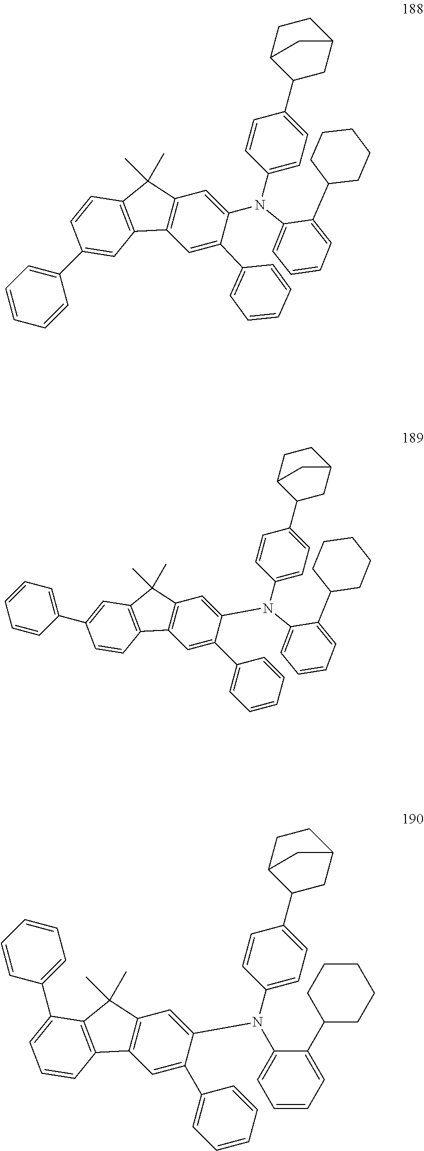 [SMILES: CC1(C)c2ccc(-c3ccccc3)cc2-c2cc(-c3ccccc3)c(N(c3ccc(C4CC5CCC4C5)cc3)c3ccccc3C3CCCCC3)cc21 ; CC1(C)c2cc(-c3ccccc3)ccc2-c2cc(-c3ccccc3)c(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)c(-c3ccccc3)cc2-c2cccc(-c3ccccc3)c21]

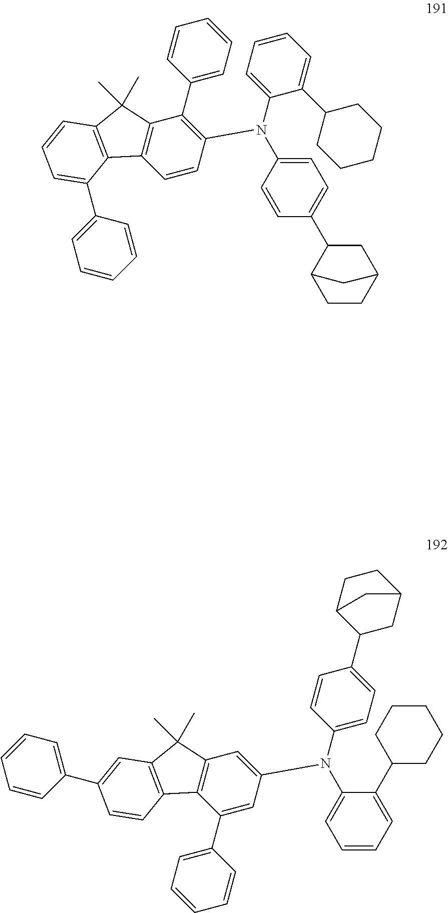 [SMILES: CC1(C)c2cccc(-c3ccccc3)c2-c2ccc(N(c3ccc(C4CC5CCC4C5)cc3)c3ccccc3C3CCCCC3)c(-c3ccccc3)c21 ; CC1(C)c2cc(-c3ccccc3)ccc2-c2c(-c3ccccc3)cc(N(c3ccc(C4CC5CCC4C5)cc3)c3ccccc3C3CCCCC3)cc21]

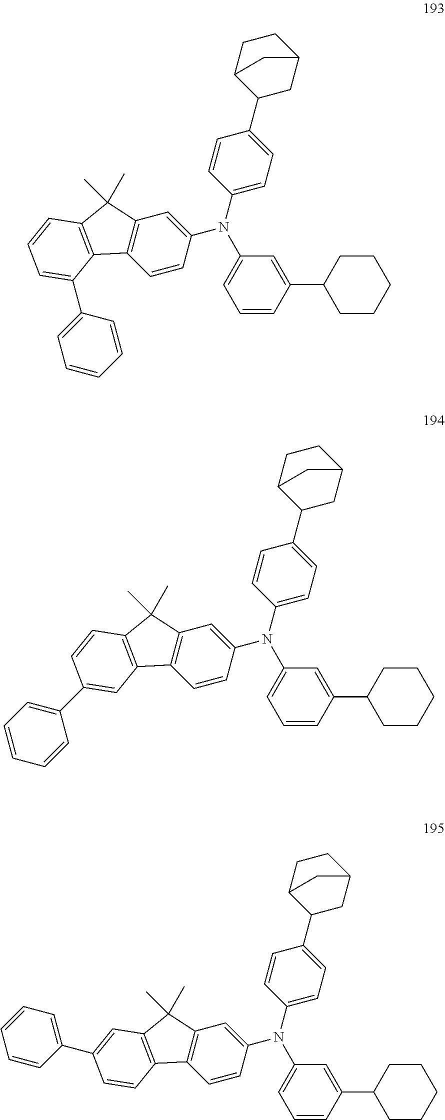 [SMILES: CC1(C)c2cc(N(c3ccc(C4CC5CCC4C5)cc3)c3cccc(C4CCCCC4)c3)ccc2-c2c(-c3ccccc3)cccc21 ; CC1(C)c2ccc(-c3ccccc3)cc2-c2ccc(N(c3ccc(C4CC5CCC4C5)cc3)c3cccc(C4CCCCC4)c3)cc21 ; CC1(C)c2cc(-c3ccccc3)ccc2-c2ccc(N(c3ccc(C4CC5CCC4C5)cc3)c3cccc(C4CCCCC4)c3)cc21]

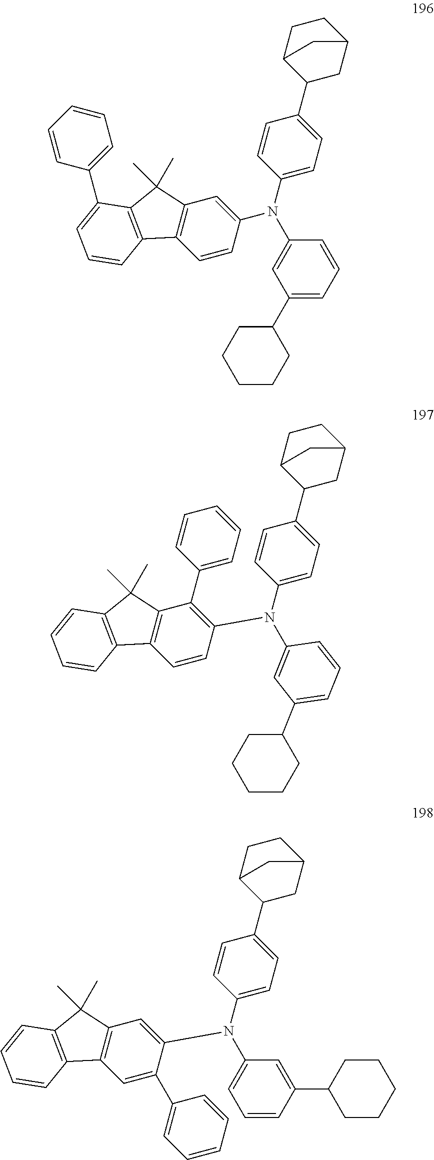

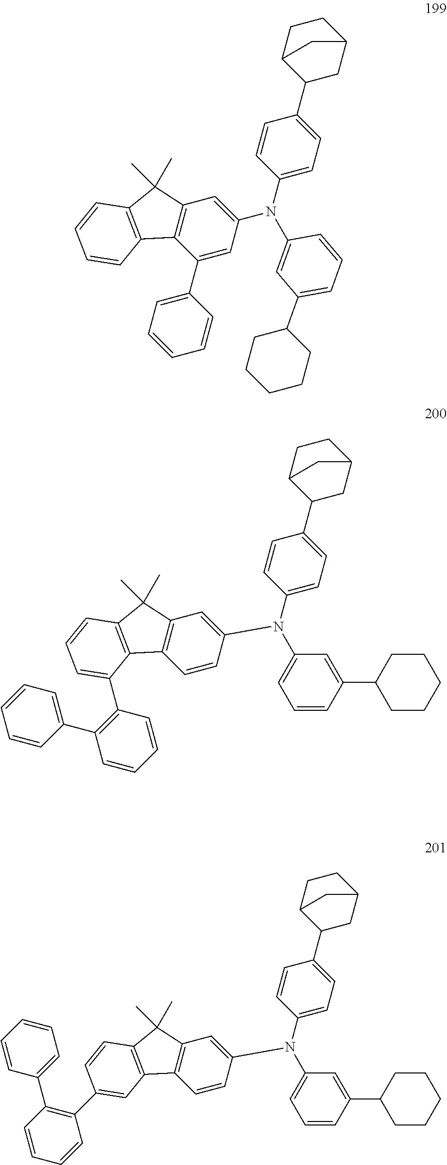 [SMILES: CC1(C)c2ccccc2-c2c(-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3cccc(C4CCCCC4)c3)cc21]

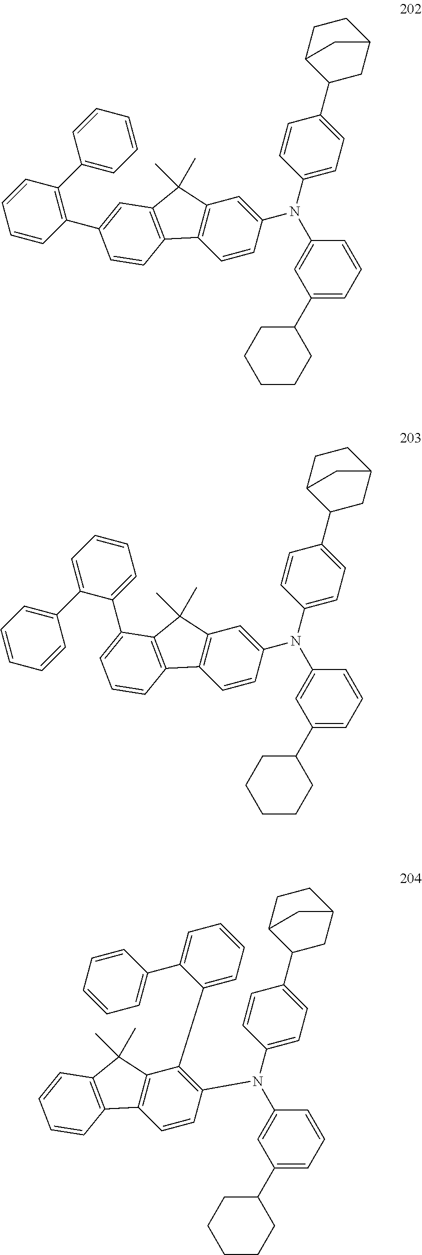 [SMILES: CC1(C)c2cc(-c3ccccc3-c3ccccc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccccc3-c3ccccc3)c21 ; CC1(C)c2ccccc2-c2ccc(N(c3ccc(C4CC5CCC4C5)cc3)c3cccc(C4CCCCC4)c3)c(-c3ccccc3-c3ccccc3)c21]

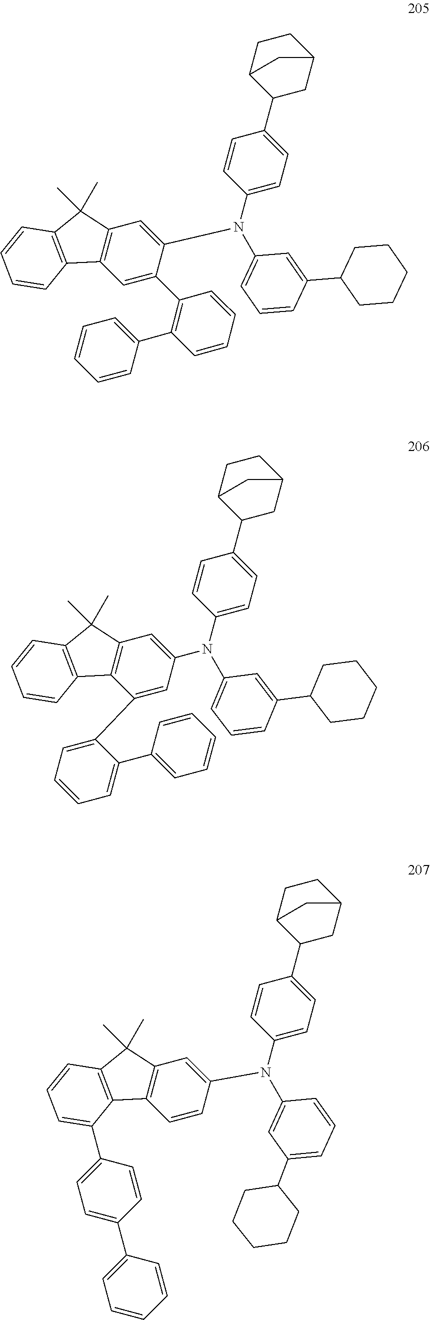 [SMILES: CC1(C)c2ccccc2-c2cc(-c3ccccc3-c3ccccc3)c(N(c3ccc(C4CC5CCC4C5)cc3)c3cccc(C4CCCCC4)c3)cc21 ; CC1(C)c2ccccc2-c2c(-c3ccccc3-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc(-c4ccccc4)cc3)cccc21]

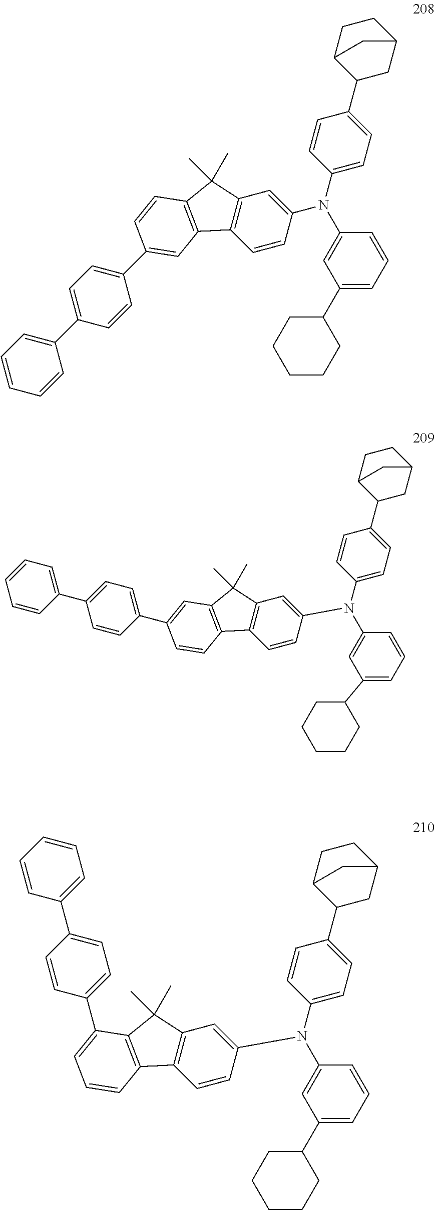 [SMILES: CC1(C)c2ccc(-c3ccc(-c4ccccc4)cc3)cc2-c2ccc(N(c3ccc(C4CC5CCC4C5)cc3)c3cccc(C4CCCCC4)c3)cc21 ; CC1(C)c2cc(-c3ccc(-c4ccccc4)cc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc(-c4ccccc4)cc3)c21]

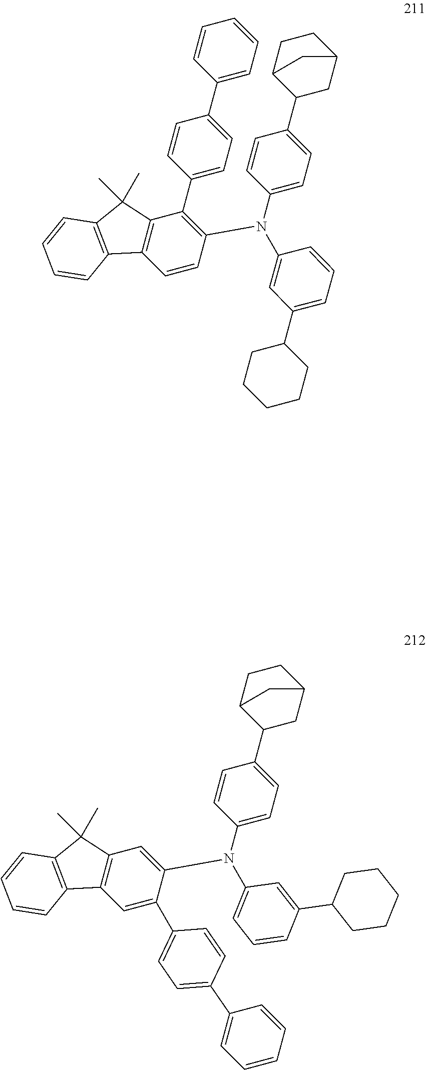 [SMILES: CC1(C)c2ccccc2-c2ccc(N(c3ccc(C4CC5CCC4C5)cc3)c3cccc(C4CCCCC4)c3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3cccc(C4CCCCC4)c3)cc21]

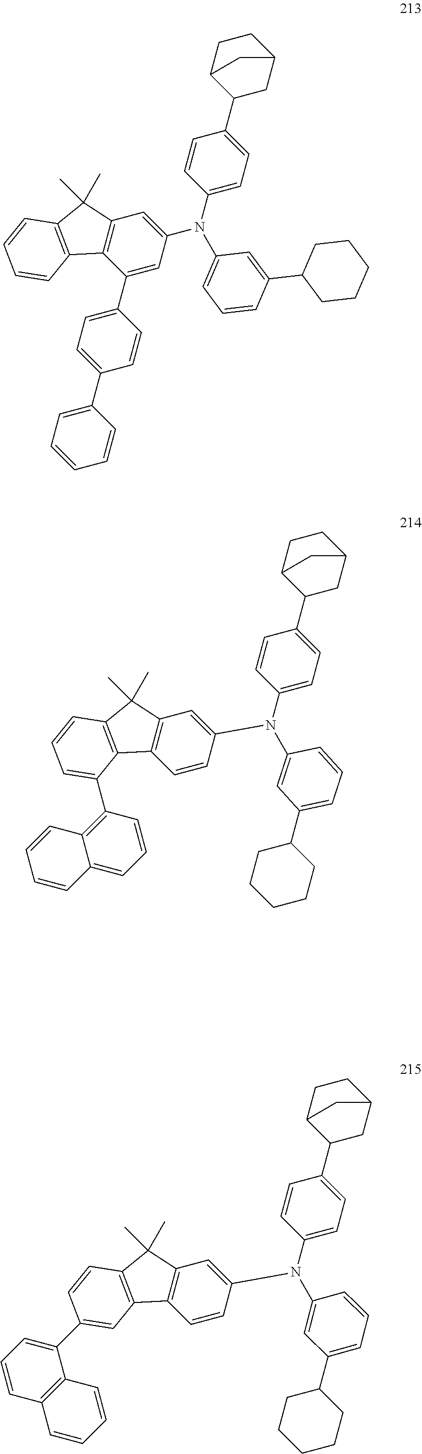 [SMILES: CC1(C)c2ccccc2-c2c(-c3ccc(-c4ccccc4)cc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cccc(C4CCCCC4)c3)cc21]

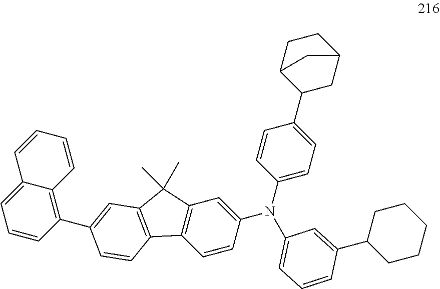 [SMILES: CC1(C)c2cc(-c3cccc4ccccc34)ccc2-c2ccc(N(c3ccc(C4CC5CCC4C5)cc3)c3cccc(C4CCCCC4)c3)cc21]

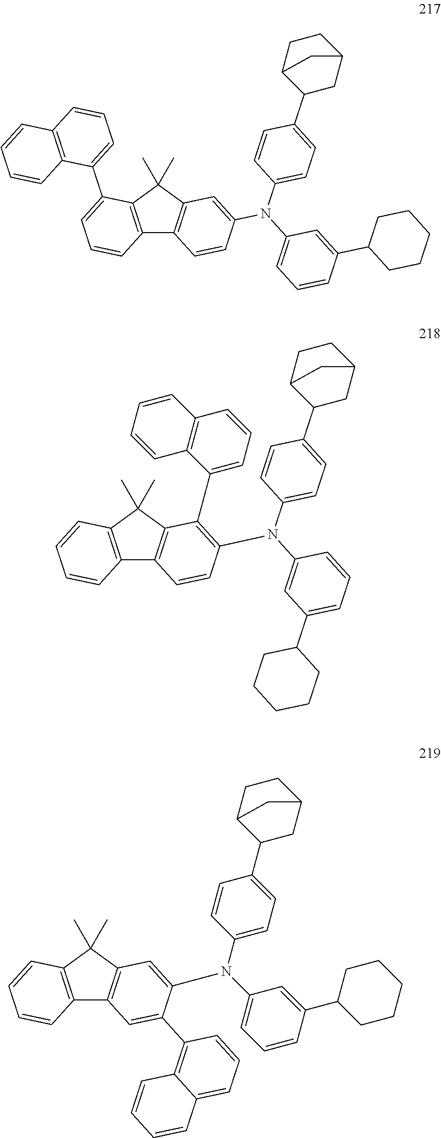 [SMILES: CC1(C)c2cc(N(c3ccc(C4CC5CCC4C5)cc3)c3cccc(C4CCCCC4)c3)ccc2-c2cccc(-c3cccc4ccccc34)c21 ; CC1(C)c2ccccc2-c2ccc(N(c3ccc(C4CC5CCC4C5)cc3)c3cccc(C4CCCCC4)c3)c(-c3cccc4ccccc34)c21 ; CC1(C)c2ccccc2-c2cc(-c3cccc4ccccc34)c(N(c3ccc(C4CC5CCC4C5)cc3)c3cccc(C4CCCCC4)c3)cc21]

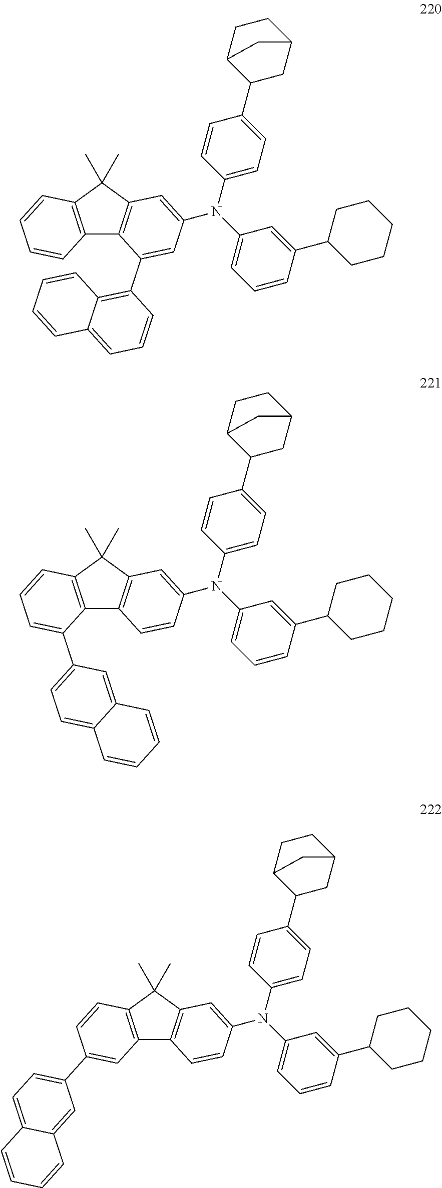 [SMILES: CC1(C)c2ccccc2-c2c(-c3cccc4ccccc34)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3cccc(C4CCCCC4)c3)cc21]

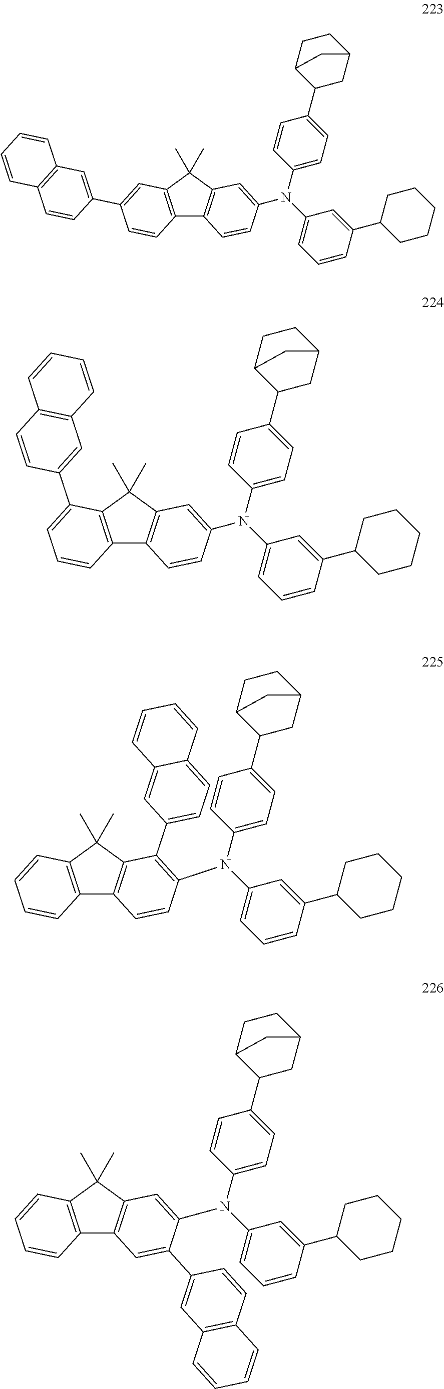 [SMILES: CC1(C)c2cc(-c3ccc4ccccc4c3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc4ccccc4c3)c21 ; CC1(C)c2ccccc2-c2ccc(N(c3ccc(C4CC5CCC4C5)cc3)c3cccc(C4CCCCC4)c3)c(-c3ccc4ccccc4c3)c21 ; CC1(C)c2ccccc2-c2cc(-c3ccc4ccccc4c3)c(N(c3ccc(C4CC5CCC4C5)cc3)c3cccc(C4CCCCC4)c3)cc21]

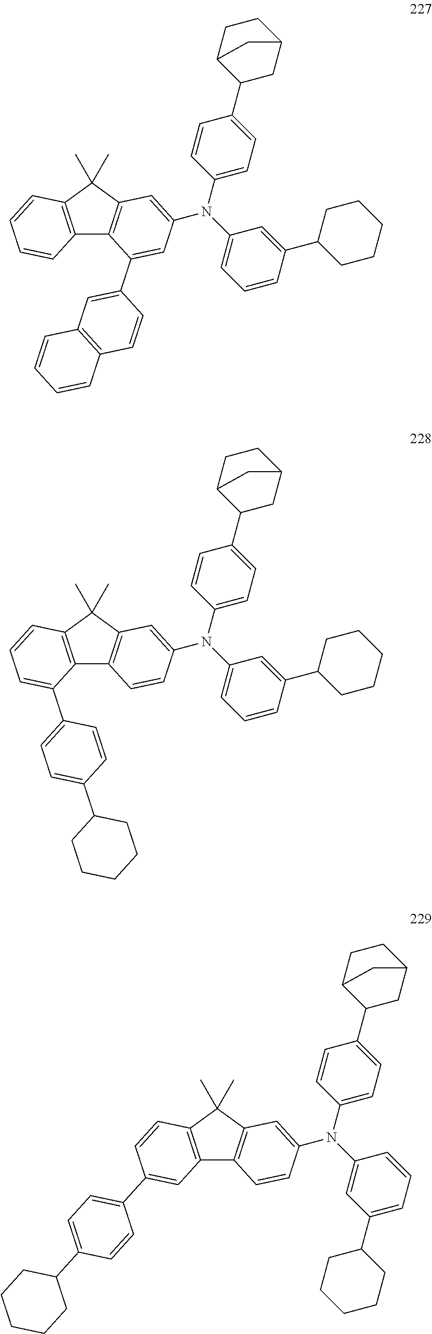 [SMILES: CC1(C)c2ccccc2-c2c(-c3ccc4ccccc4c3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cccc(C4CCCCC4)c3)cc21]

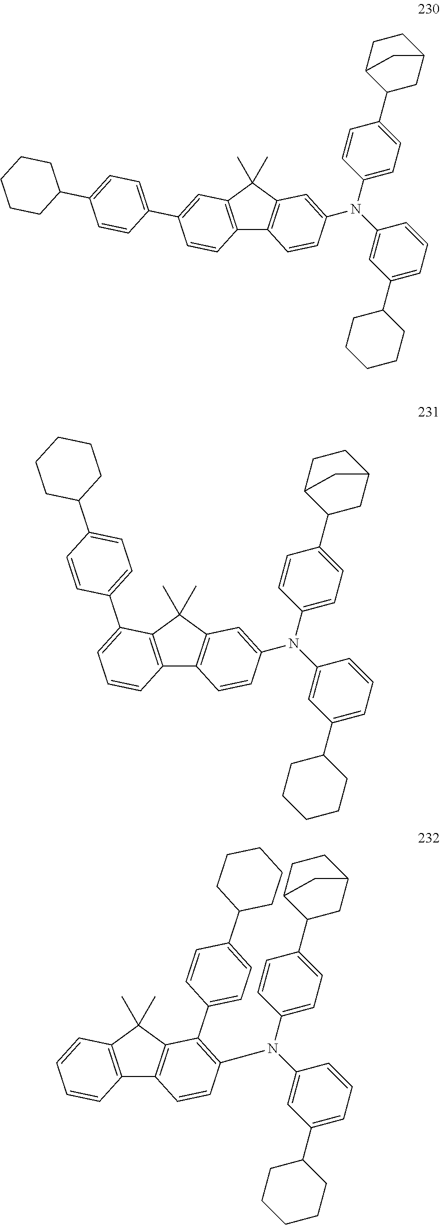

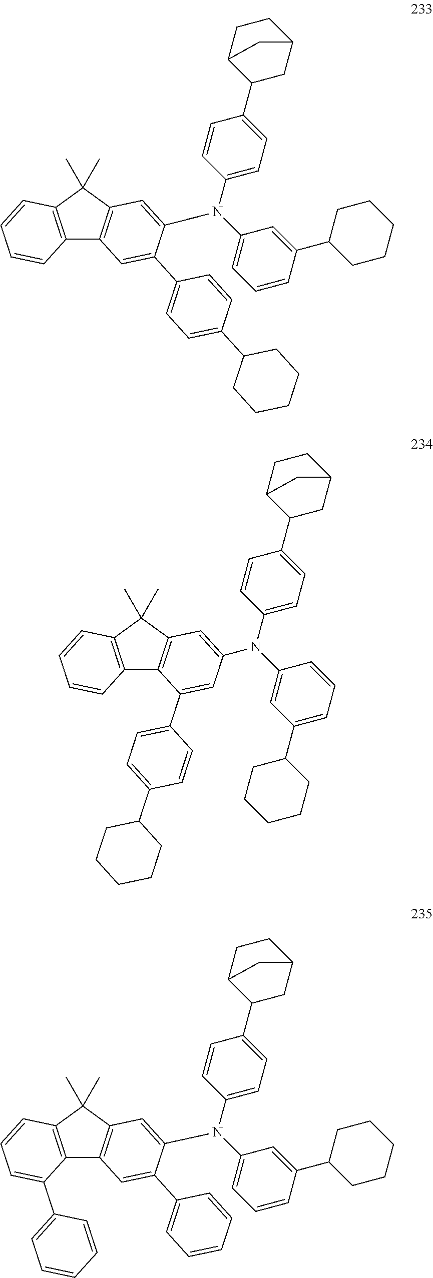 [SMILES: CC1(C)c2ccccc2-c2cc(-c3ccc(C4CCCCC4)cc3)c(N(c3ccc(C4CC5CCC4C5)cc3)c3cccc(C4CCCCC4)c3)cc21 ; CC1(C)c2ccccc2-c2c(-c3ccc(C4CCCCC4)cc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)c(-c3ccccc3)cc2-c2c(-c3ccccc3)cccc21]

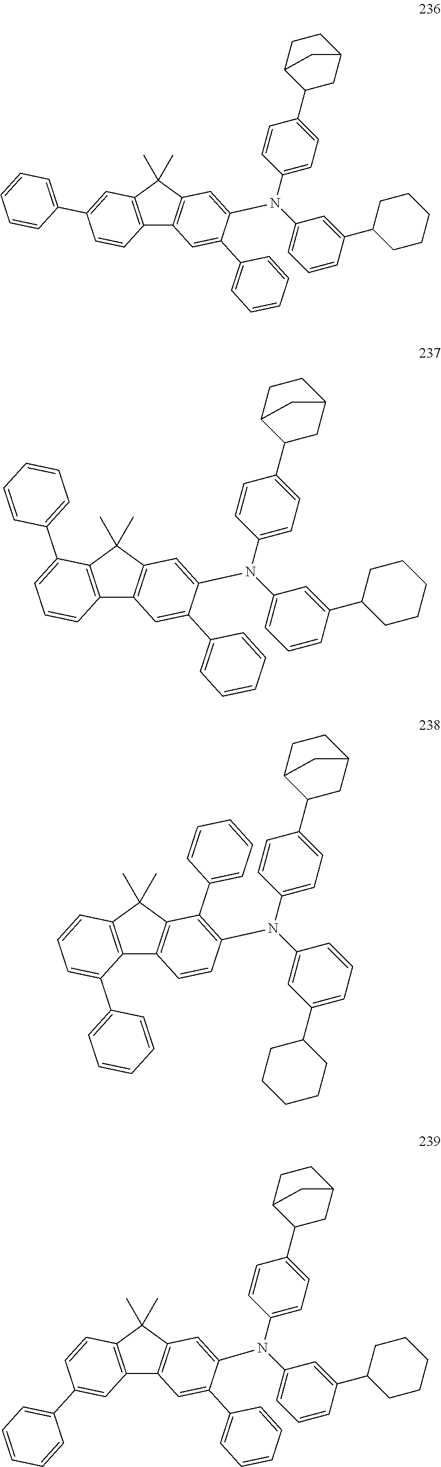 [SMILES: CC1(C)c2cc(-c3ccccc3)ccc2-c2cc(-c3ccccc3)c(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3cccc(C4CCCCC4)c3)c(-c3ccccc3)c21 ; CC1(C)c2ccc(-c3ccccc3)cc2-c2cc(-c3ccccc3)c(N(c3ccc(C4CC5CCC4C5)cc3)c3cccc(C4CCCCC4)c3)cc21]

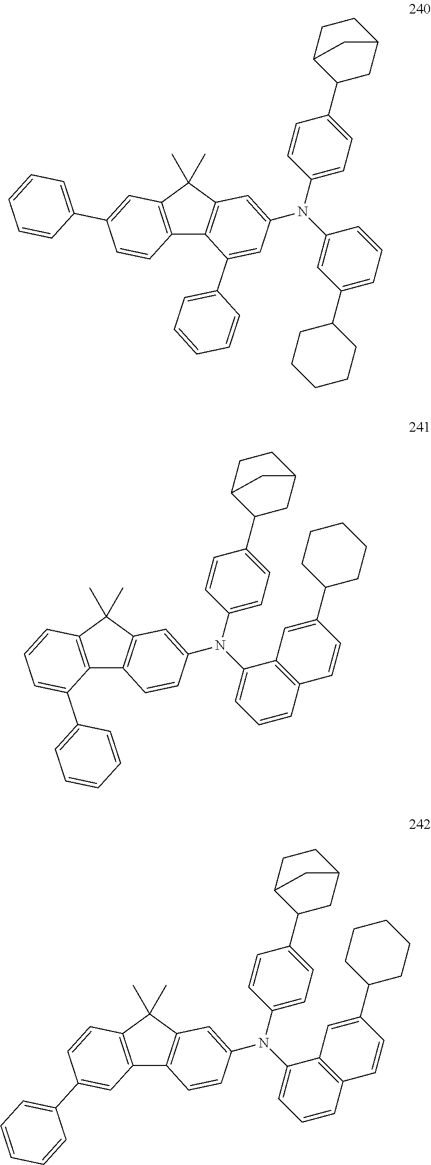 [SMILES: CC1(C)c2cc(-c3ccccc3)ccc2-c2c(-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc4ccc(C5CCCCC5)cc34)ccc2-c2c(-c3ccccc3)cccc21 ; CC1(C)c2ccc(-c3ccccc3)cc2-c2ccc(N(c3ccc(C4CC5CCC4C5)cc3)c3cccc4ccc(C5CCCCC5)cc34)cc21]

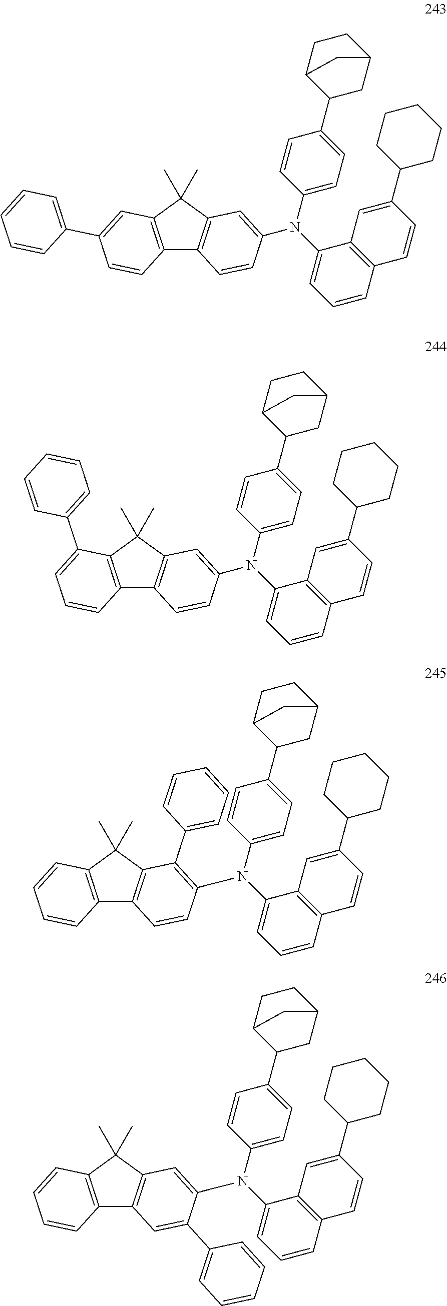

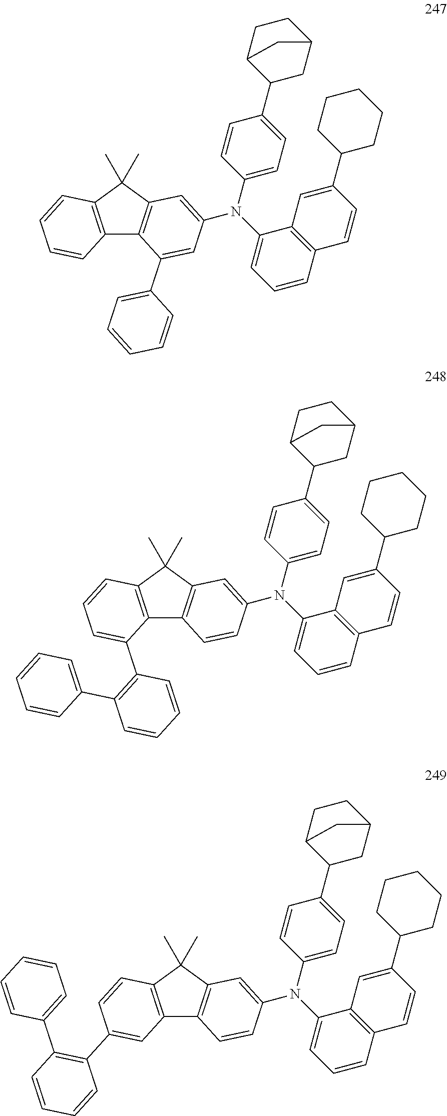 [SMILES: CC1(C)c2ccccc2-c2c(-c3ccccc3)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3cccc4ccc(C5CCCCC5)cc34)cc21]

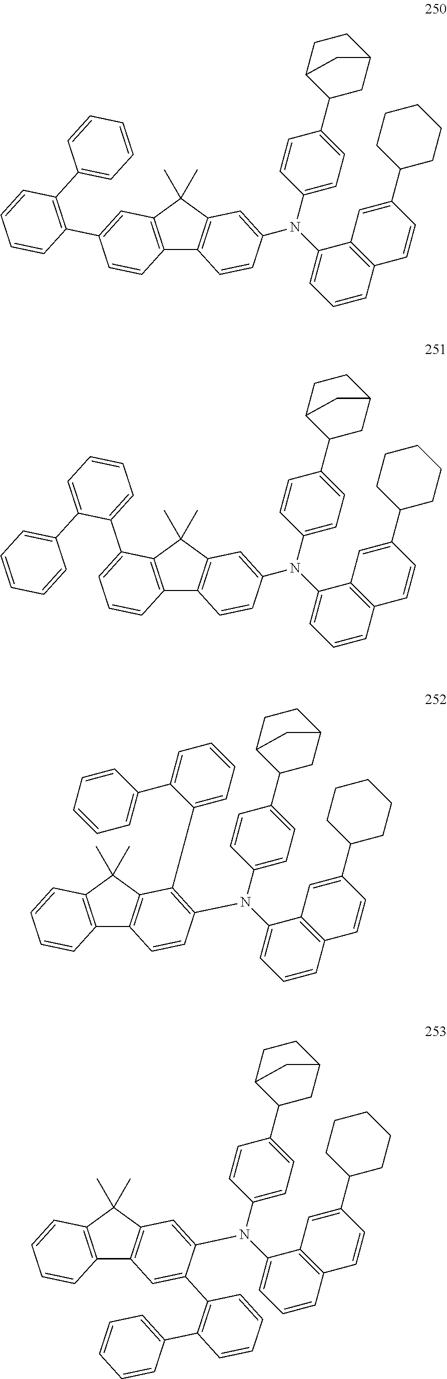 [SMILES: CC1(C)c2cc(-c3ccccc3-c3ccccc3)ccc2-c2ccc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2cccc(-c3ccccc3-c3ccccc3)c21 ; CC1(C)c2ccccc2-c2ccc(N(c3ccc(C4CC5CCC4C5)cc3)c3cccc4ccc(C5CCCCC5)cc34)c(-c3ccccc3-c3ccccc3)c21 ; CC1(C)c2ccccc2-c2cc(-c3ccccc3-c3ccccc3)c(N(c3ccc(C4CC5CCC4C5)cc3)c3cccc4ccc(C5CCCCC5)cc34)cc21]

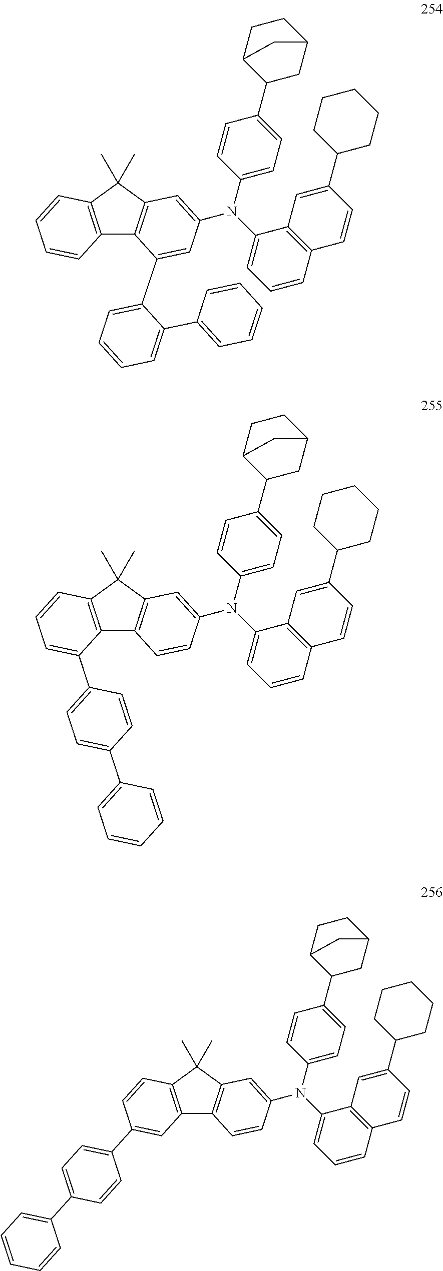 [SMILES: CC1(C)c2ccccc2-c2c(-c3ccccc3-c3ccccc3)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3cccc4ccc(C5CCCCC5)cc34)cc21]

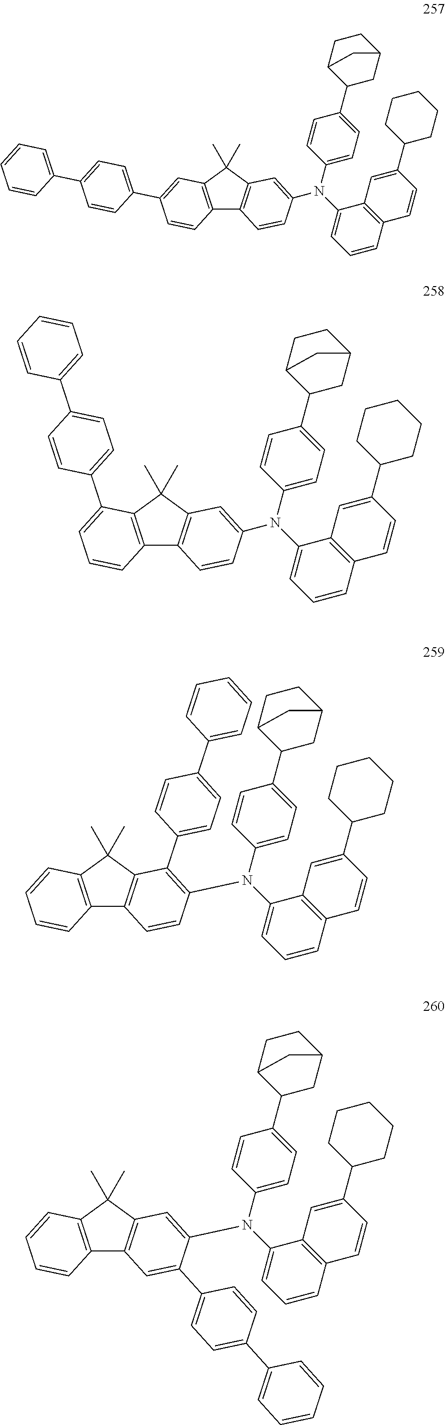 [SMILES: CC1(C)c2cc(-c3ccc(-c4ccccc4)cc3)ccc2-c2ccc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2cccc(-c3ccc(-c4ccccc4)cc3)c21 ; CC1(C)c2ccccc2-c2ccc(N(c3ccc(C4CC5CCC4C5)cc3)c3cccc4ccc(C5CCCCC5)cc34)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3cccc4ccc(C5CCCCC5)cc34)cc21]

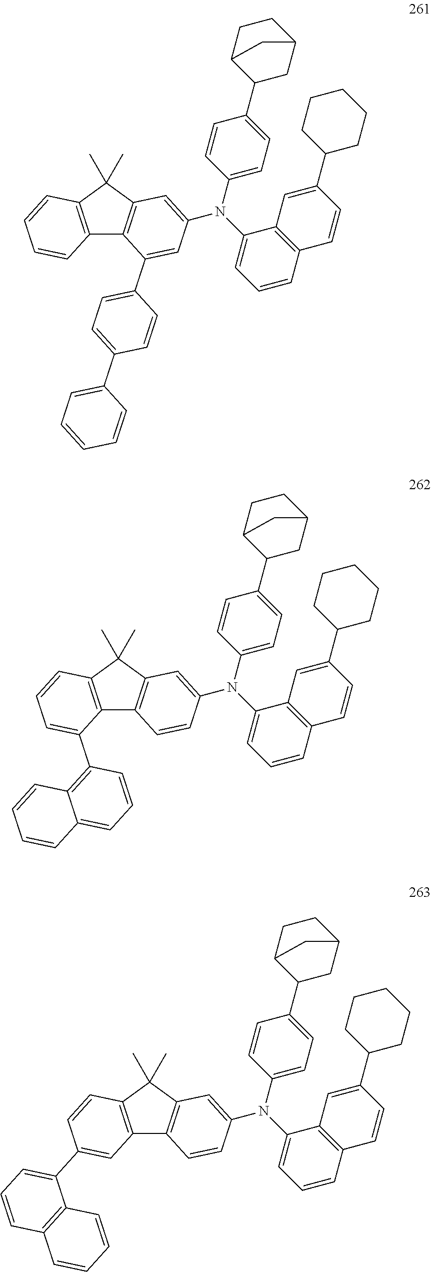 [SMILES: CC1(C)c2ccccc2-c2c(-c3ccc(-c4ccccc4)cc3)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cccc4ccc(C5CCCCC5)cc34)cc21]

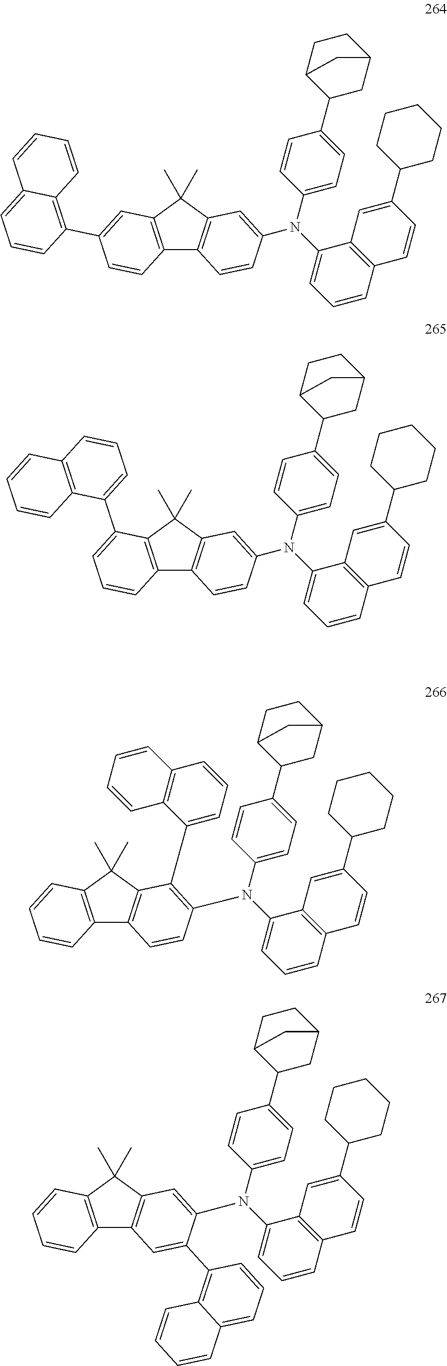

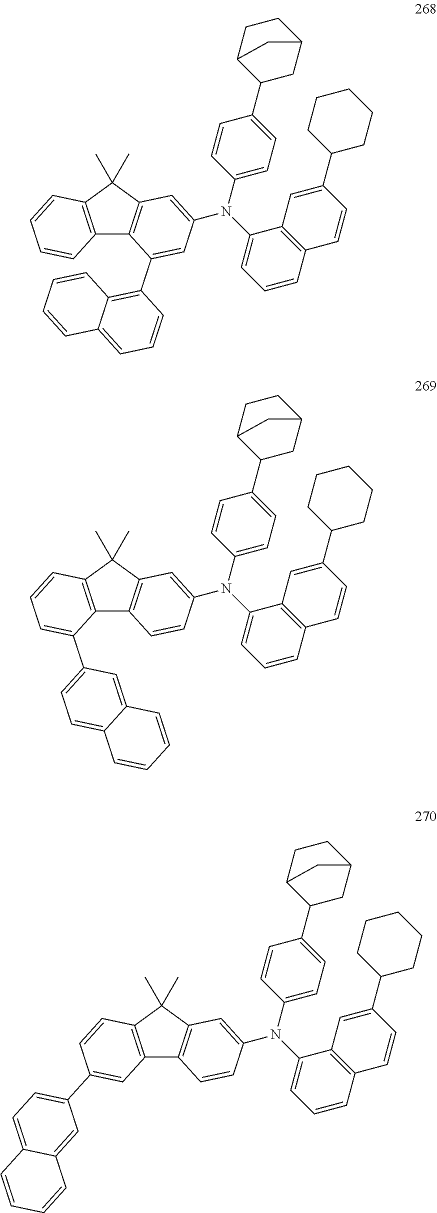 [SMILES: CC1(C)c2ccccc2-c2c(-c3cccc4ccccc34)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3cccc4ccc(C5CCCCC5)cc34)cc21]

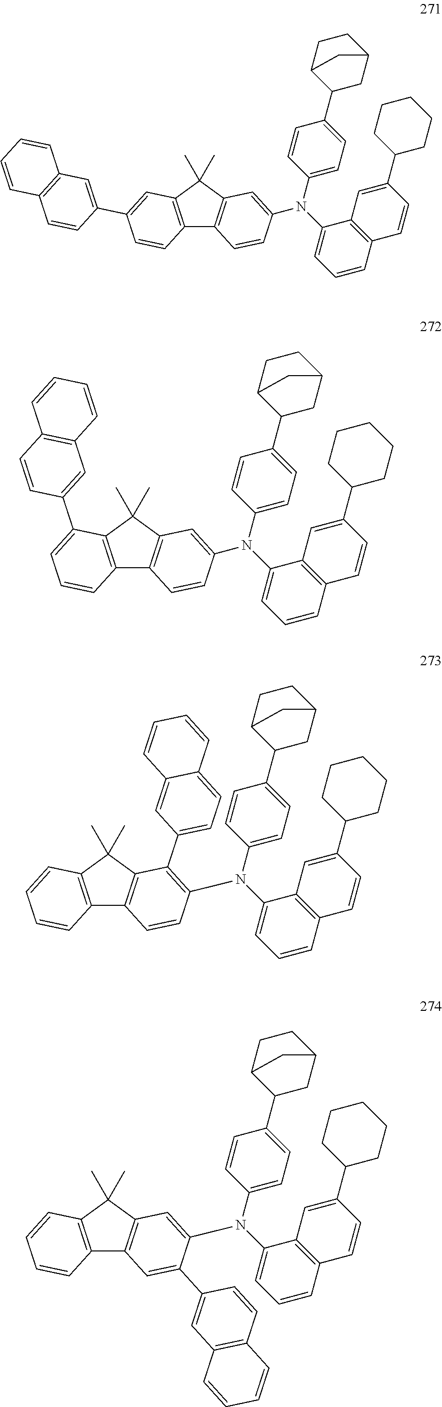 [SMILES: CC1(C)c2cc(-c3ccc4ccccc4c3)ccc2-c2ccc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2cccc(-c3ccc4ccccc4c3)c21 ; CC1(C)c2ccccc2-c2ccc(N(c3ccc(C4CC5CCC4C5)cc3)c3cccc4ccc(C5CCCCC5)cc34)c(-c3ccc4ccccc4c3)c21 ; CC1(C)c2ccccc2-c2cc(-c3ccc4ccccc4c3)c(N(c3ccc(C4CC5CCC4C5)cc3)c3cccc4ccc(C5CCCCC5)cc34)cc21]

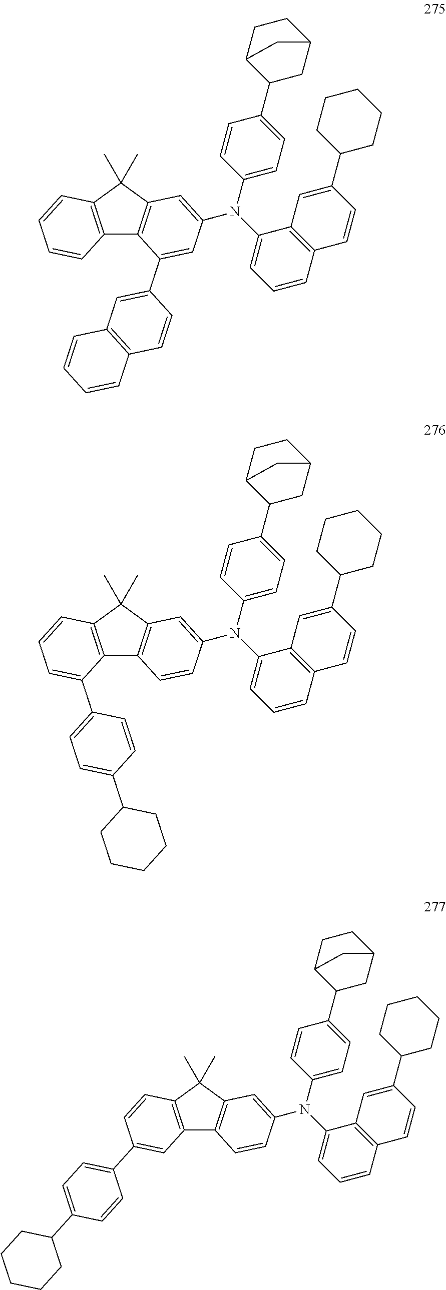 [SMILES: CC1(C)c2ccccc2-c2c(-c3ccc4ccccc4c3)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cccc4ccc(C5CCCCC5)cc34)cc21]

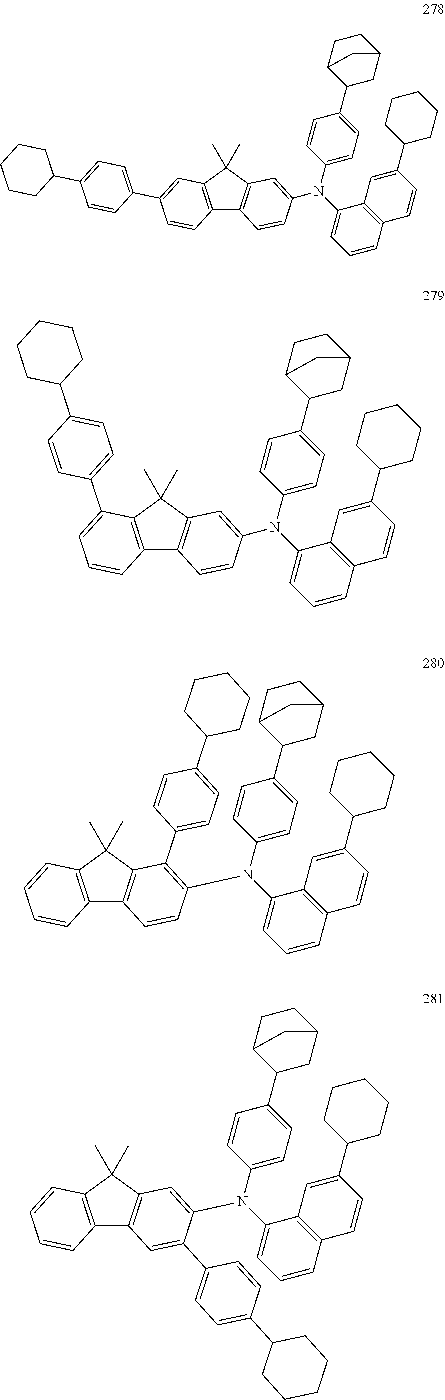 [SMILES: CC1(C)c2cc(-c3ccc(C4CCCCC4)cc3)ccc2-c2ccc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)ccc2-c2cccc(-c3ccc(C4CCCCC4)cc3)c21 ; CC1(C)c2ccccc2-c2ccc(N(c3ccc(C4CC5CCC4C5)cc3)c3cccc4ccc(C5CCCCC5)cc34)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3cccc4ccc(C5CCCCC5)cc34)cc21]

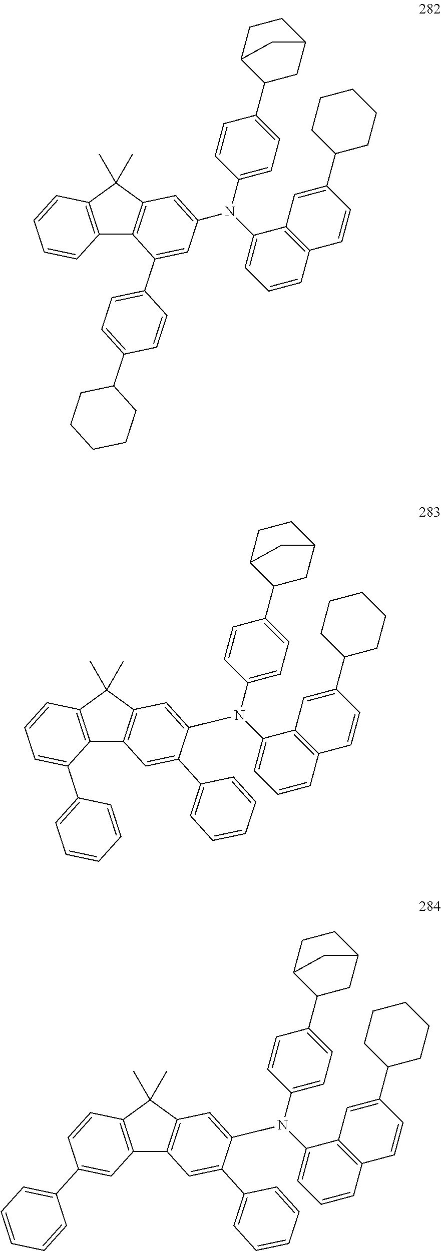 [SMILES: CC1(C)c2ccccc2-c2c(-c3ccc(C4CCCCC4)cc3)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)c(-c3ccccc3)cc2-c2c(-c3ccccc3)cccc21 ; CC1(C)c2ccc(-c3ccccc3)cc2-c2cc(-c3ccccc3)c(N(c3ccc(C4CC5CCC4C5)cc3)c3cccc4ccc(C5CCCCC5)cc34)cc21]

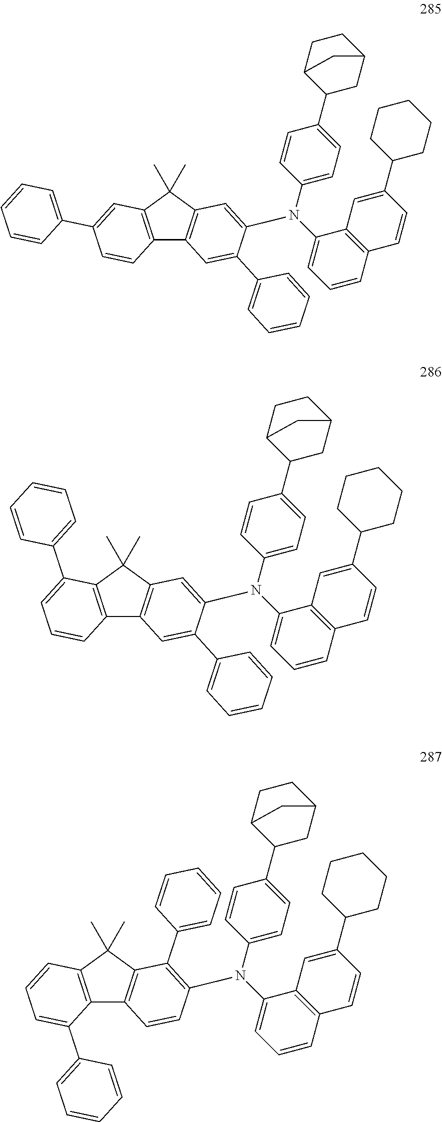 [SMILES: CC1(C)c2cc(-c3ccccc3)ccc2-c2cc(-c3ccccc3)c(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4ccc(C5CCCCC5)cc34)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3cccc4ccc(C5CCCCC5)cc34)c(-c3ccccc3)c21]

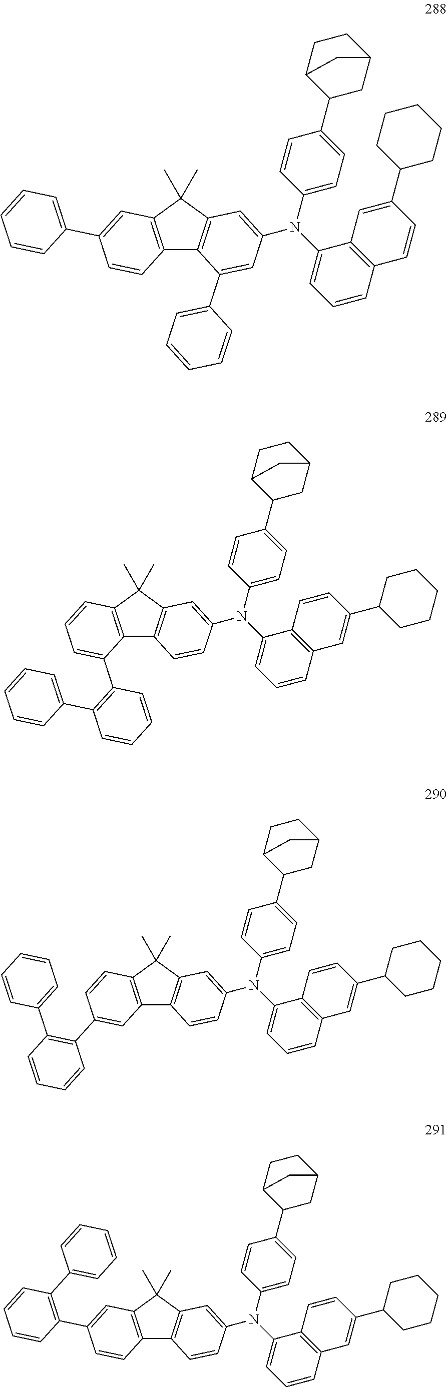 [SMILES: CC1(C)c2cc(-c3ccccc3)ccc2-c2c(-c3ccccc3)cc(N(c3ccc(C4CC5CCC4C5)cc3)c3cccc4ccc(C5CCCCC5)cc34)cc21 ; CC1(C)c2cc(N(c3ccc(C4CC5CCC4C5)cc3)c3cccc4cc(C5CCCCC5)ccc34)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3cccc4cc(C5CCCCC5)ccc34)cc21 ; CC1(C)c2cc(-c3ccccc3-c3ccccc3)ccc2-c2ccc(N(c3ccc(C4CC5CCC4C5)cc3)c3cccc4cc(C5CCCCC5)ccc34)cc21]

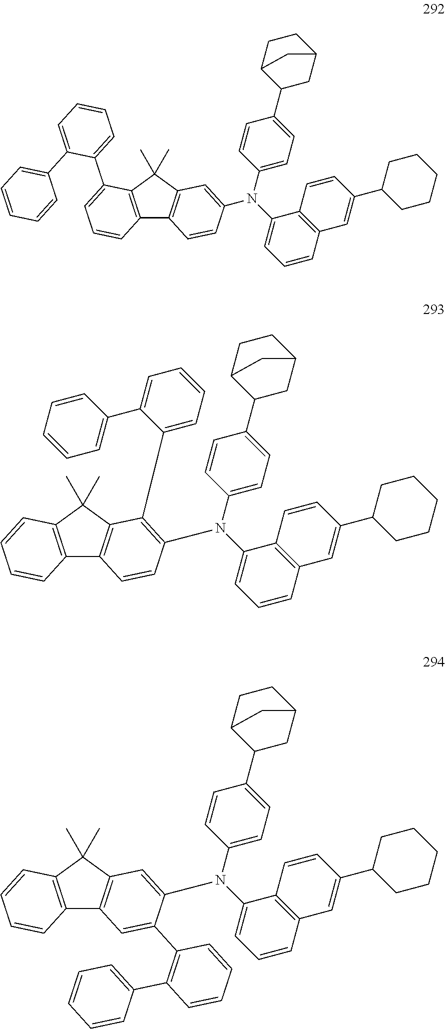 [SMILES: CC1(C)c2cc(N(c3ccc(C4CC5CCC4C5)cc3)c3cccc4cc(C5CCCCC5)ccc34)ccc2-c2cccc(-c3ccccc3-c3ccccc3)c21 ; CC1(C)c2ccccc2-c2ccc(N(c3ccc(C4CC5CCC4C5)cc3)c3cccc4cc(C5CCCCC5)ccc34)c(-c3ccccc3-c3ccccc3)c21 ; CC1(C)c2ccccc2-c2cc(-c3ccccc3-c3ccccc3)c(N(c3ccc(C4CC5CCC4C5)cc3)c3cccc4cc(C5CCCCC5)ccc34)cc21]

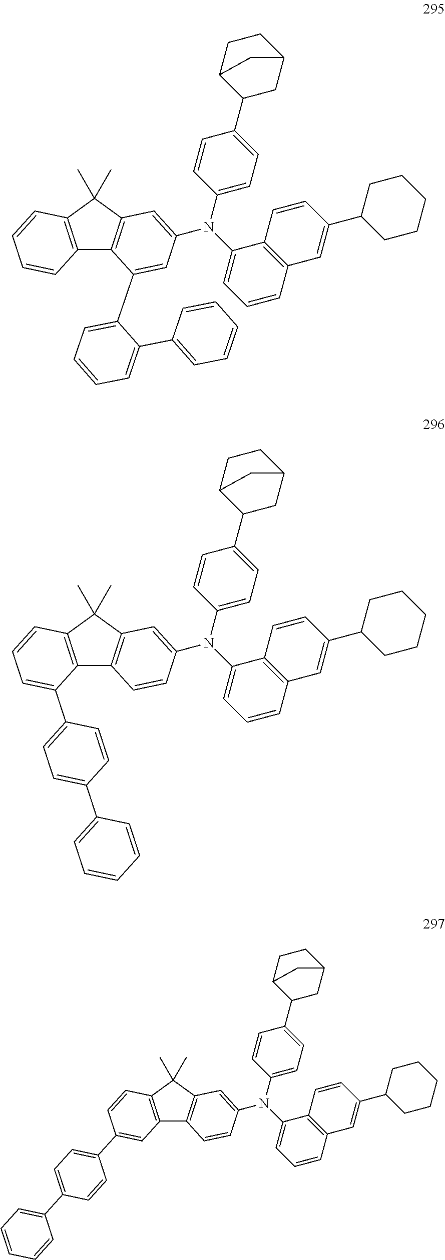 [SMILES: CC1(C)c2ccccc2-c2c(-c3ccccc3-c3ccccc3)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3cccc4cc(C5CCCCC5)ccc34)cc21]

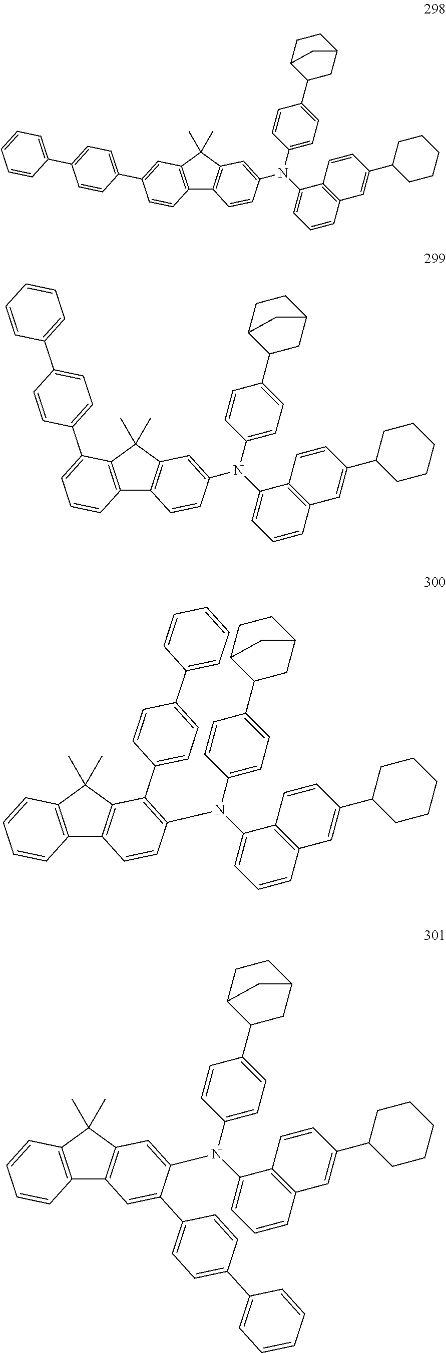 [SMILES: CC1(C)c2cc(-c3ccc(-c4ccccc4)cc3)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3ccc(-c4ccccc4)cc3)c21 ; CC1(C)c2ccccc2-c2ccc(N(c3ccc(C4CC5CCC4C5)cc3)c3cccc4cc(C5CCCCC5)ccc34)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3cccc4cc(C5CCCCC5)ccc34)cc21]

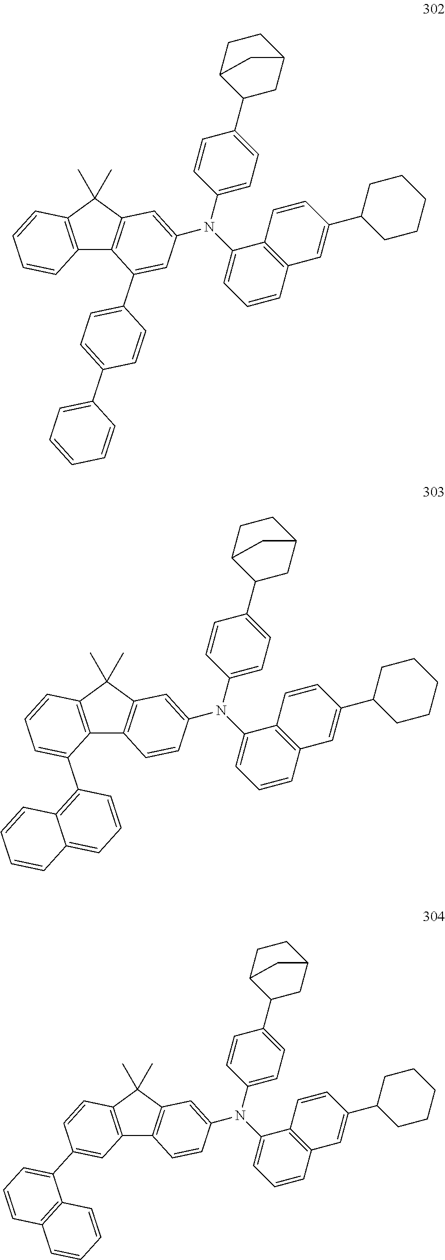 [SMILES: CC1(C)c2ccccc2-c2c(-c3ccc(-c4ccccc4)cc3)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cccc4cc(C5CCCCC5)ccc34)cc21]

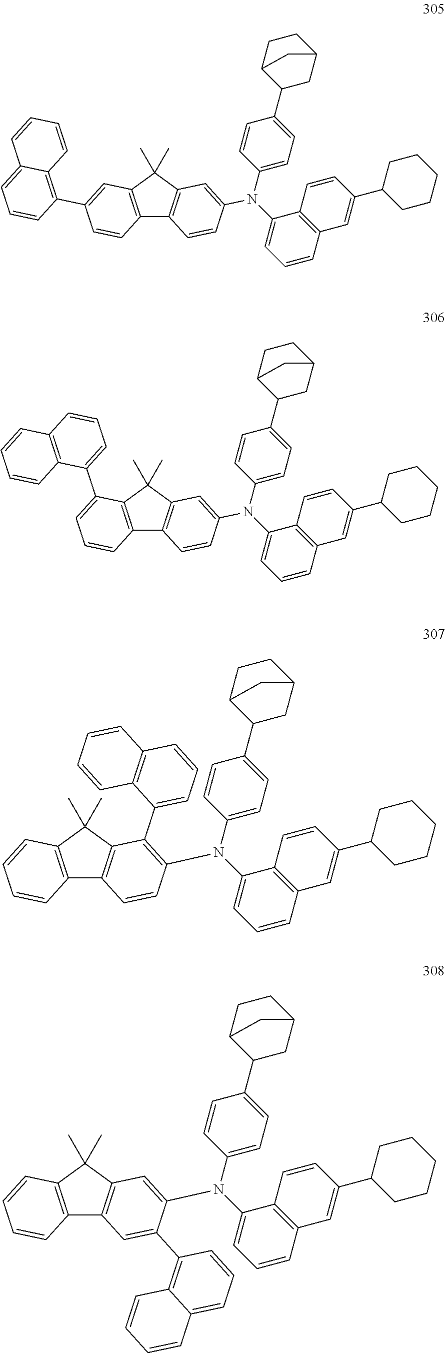 [SMILES: CC1(C)c2cc(-c3cccc4ccccc34)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3cccc4ccccc34)c21 ; CC1(C)c2ccccc2-c2ccc(N(c3ccc(C4CC5CCC4C5)cc3)c3cccc4cc(C5CCCCC5)ccc34)c(-c3cccc4ccccc34)c21 ; CC1(C)c2ccccc2-c2cc(-c3cccc4ccccc34)c(N(c3ccc(C4CC5CCC4C5)cc3)c3cccc4cc(C5CCCCC5)ccc34)cc21]

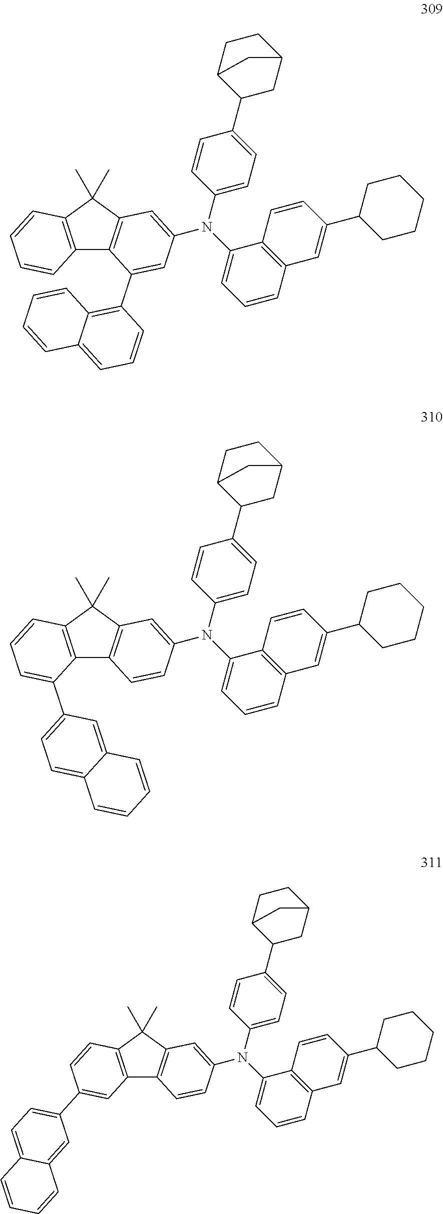 [SMILES: CC1(C)c2ccccc2-c2c(-c3cccc4ccccc34)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3cccc4cc(C5CCCCC5)ccc34)cc21]

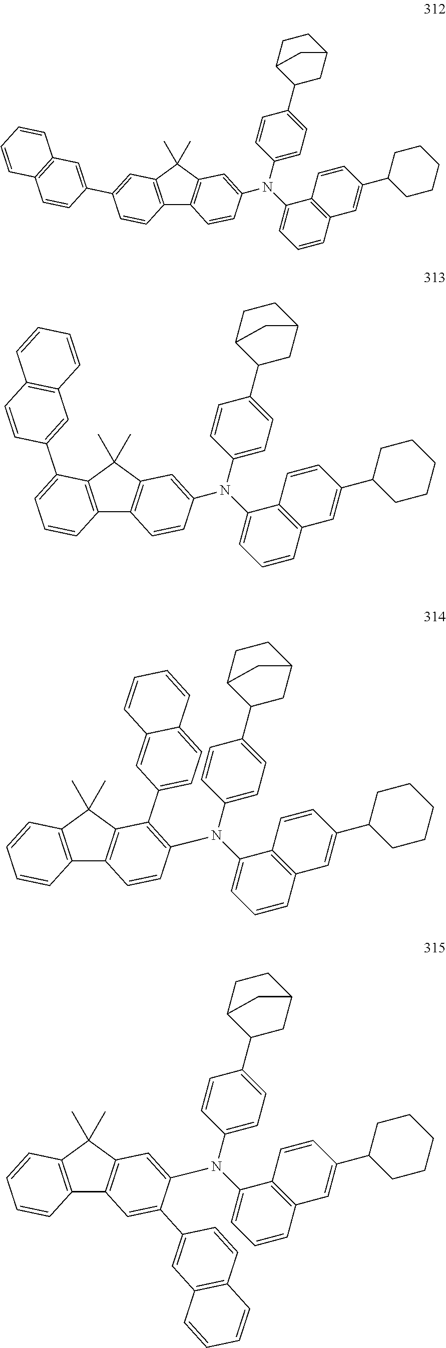 [SMILES: CC1(C)c2cc(-c3ccc4ccccc4c3)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3ccc4ccccc4c3)c21 ; CC1(C)c2ccccc2-c2ccc(N(c3ccc(C4CC5CCC4C5)cc3)c3cccc4cc(C5CCCCC5)ccc34)c(-c3ccc4ccccc4c3)c21 ; CC1(C)c2ccccc2-c2cc(-c3ccc4ccccc4c3)c(N(c3ccc(C4CC5CCC4C5)cc3)c3cccc4cc(C5CCCCC5)ccc34)cc21]

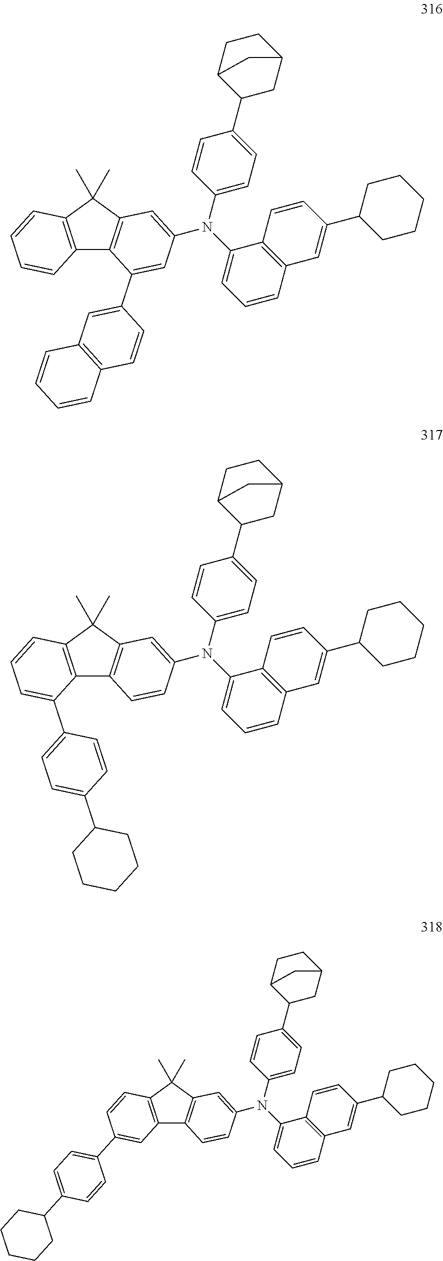 [SMILES: CC1(C)c2ccccc2-c2c(-c3ccc4ccccc4c3)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cccc4cc(C5CCCCC5)ccc34)cc21]

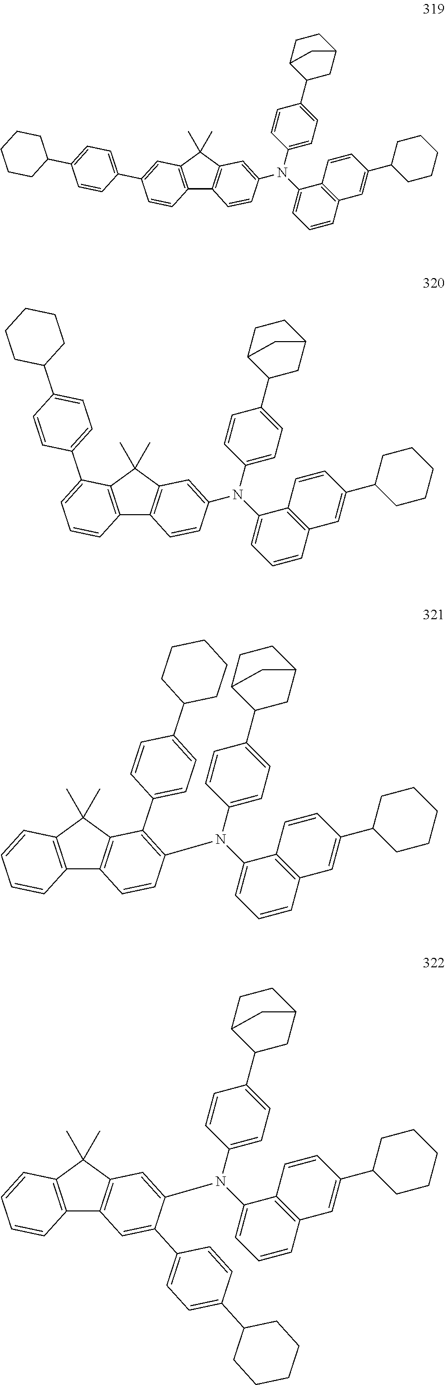 [SMILES: CC1(C)c2cc(-c3ccc(C4CCCCC4)cc3)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3ccc(C4CCCCC4)cc3)c21 ; CC1(C)c2ccccc2-c2ccc(N(c3ccc(C4CC5CCC4C5)cc3)c3cccc4cc(C5CCCCC5)ccc34)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3cccc4cc(C5CCCCC5)ccc34)cc21]

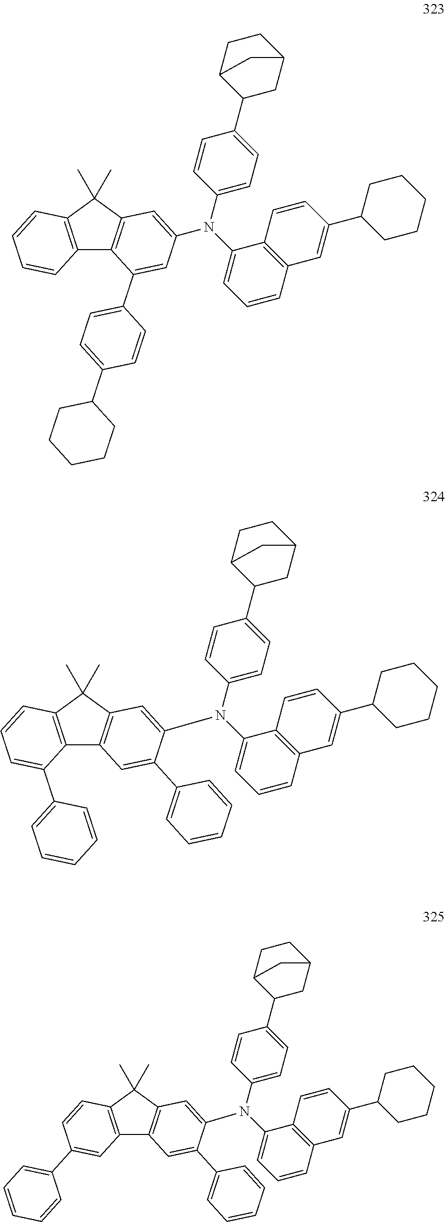 [SMILES: CC1(C)c2ccccc2-c2c(-c3ccc(C4CCCCC4)cc3)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)c(-c3ccccc3)cc2-c2c(-c3ccccc3)cccc21 ; CC1(C)c2ccc(-c3ccccc3)cc2-c2cc(-c3ccccc3)c(N(c3ccc(C4CC5CCC4C5)cc3)c3cccc4cc(C5CCCCC5)ccc34)cc21]

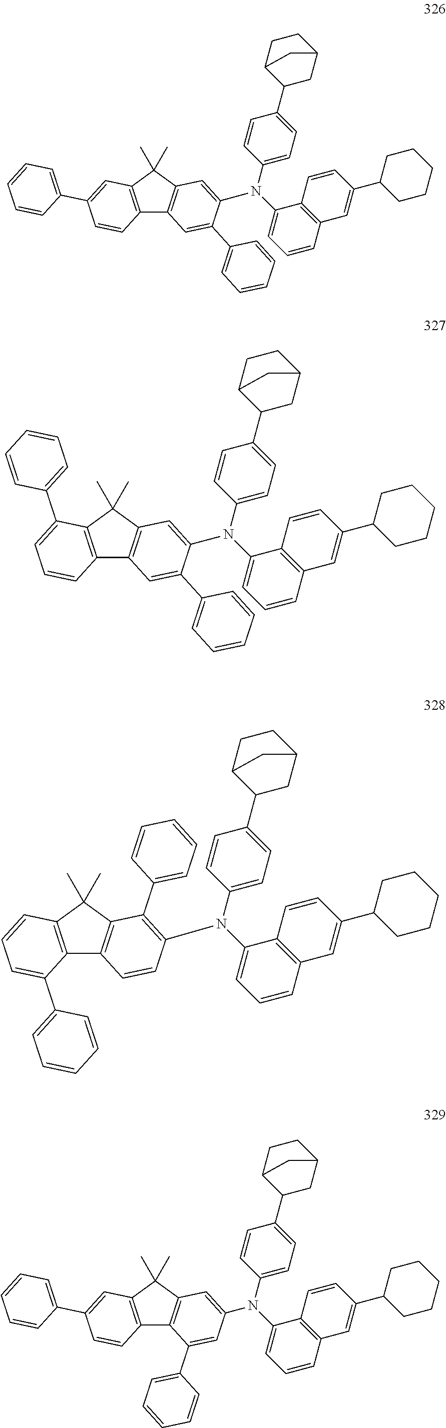 [SMILES: CC1(C)c2cc(-c3ccccc3)ccc2-c2cc(-c3ccccc3)c(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3cccc4cc(C5CCCCC5)ccc34)c(-c3ccccc3)c21 ; CC1(C)c2cc(-c3ccccc3)ccc2-c2c(-c3ccccc3)cc(N(c3ccc(C4CC5CCC4C5)cc3)c3cccc4cc(C5CCCCC5)ccc34)cc21]

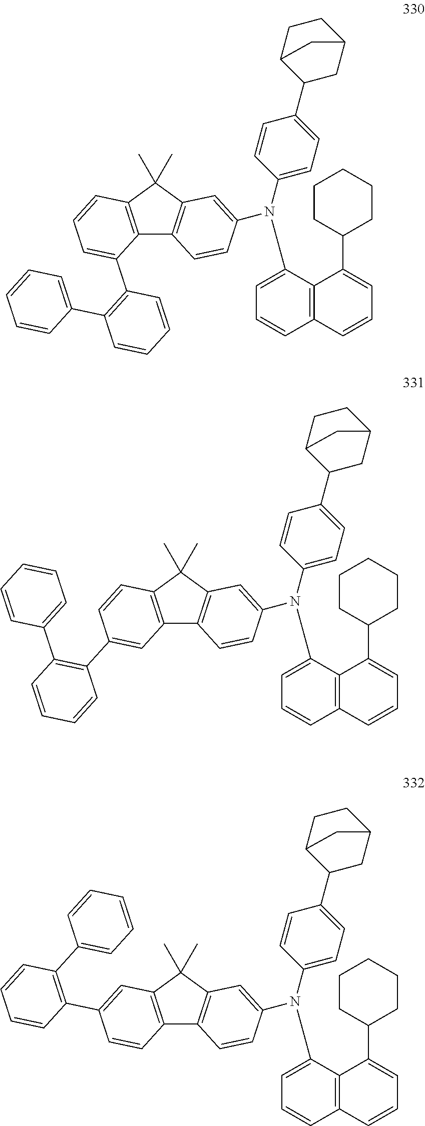 [SMILES: CC1(C)c2cc(N(c3ccc(C4CC5CCC4C5)cc3)c3cccc4cccc(C5CCCCC5)c34)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3cccc4cccc(C5CCCCC5)c34)cc21 ; CC1(C)c2cc(-c3ccccc3-c3ccccc3)ccc2-c2ccc(N(c3ccc(C4CC5CCC4C5)cc3)c3cccc4cccc(C5CCCCC5)c34)cc21]

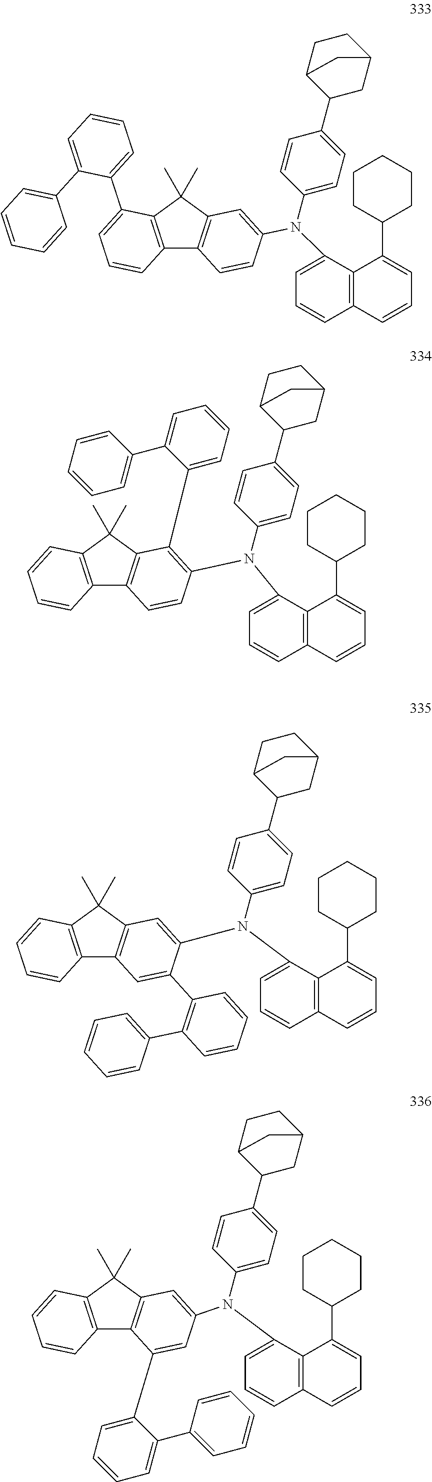 [SMILES: CC1(C)c2cc(N(c3ccc(C4CC5CCC4C5)cc3)c3cccc4cccc(C5CCCCC5)c34)ccc2-c2cccc(-c3ccccc3-c3ccccc3)c21 ; CC1(C)c2ccccc2-c2ccc(N(c3ccc(C4CC5CCC4C5)cc3)c3cccc4cccc(C5CCCCC5)c34)c(-c3ccccc3-c3ccccc3)c21 ; CC1(C)c2ccccc2-c2cc(-c3ccccc3-c3ccccc3)c(N(c3ccc(C4CC5CCC4C5)cc3)c3cccc4cccc(C5CCCCC5)c34)cc21 ; CC1(C)c2ccccc2-c2c(-c3ccccc3-c3ccccc3)cc(N(c3ccc(C4CC5CCC4C5)cc3)c3cccc4cccc(C5CCCCC5)c34)cc21]

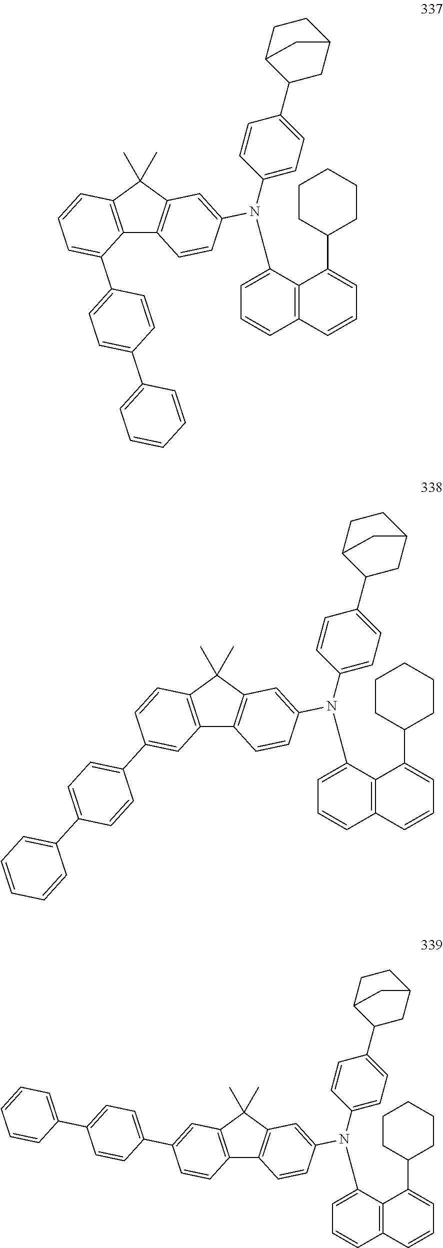 [SMILES: CC1(C)c2cc(N(c3ccc(C4CC5CCC4C5)cc3)c3cccc4cccc(C5CCCCC5)c34)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3cccc4cccc(C5CCCCC5)c34)cc21 ; CC1(C)c2cc(-c3ccc(-c4ccccc4)cc3)ccc2-c2ccc(N(c3ccc(C4CC5CCC4C5)cc3)c3cccc4cccc(C5CCCCC5)c34)cc21]

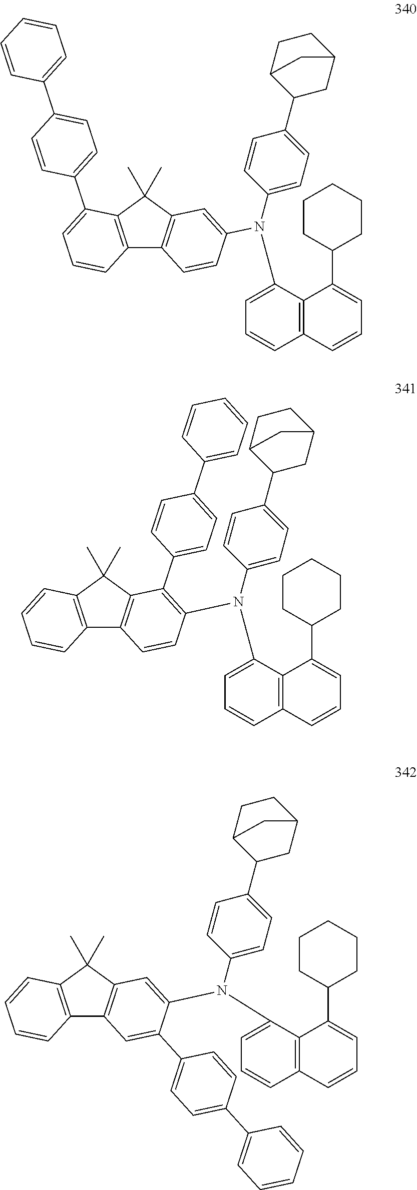 [SMILES: CC1(C)c2cc(N(c3ccc(C4CC5CCC4C5)cc3)c3cccc4cccc(C5CCCCC5)c34)ccc2-c2cccc(-c3ccc(-c4ccccc4)cc3)c21 ; CC1(C)c2ccccc2-c2ccc(N(c3ccc(C4CC5CCC4C5)cc3)c3cccc4cccc(C5CCCCC5)c34)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3cccc4cccc(C5CCCCC5)c34)cc21]

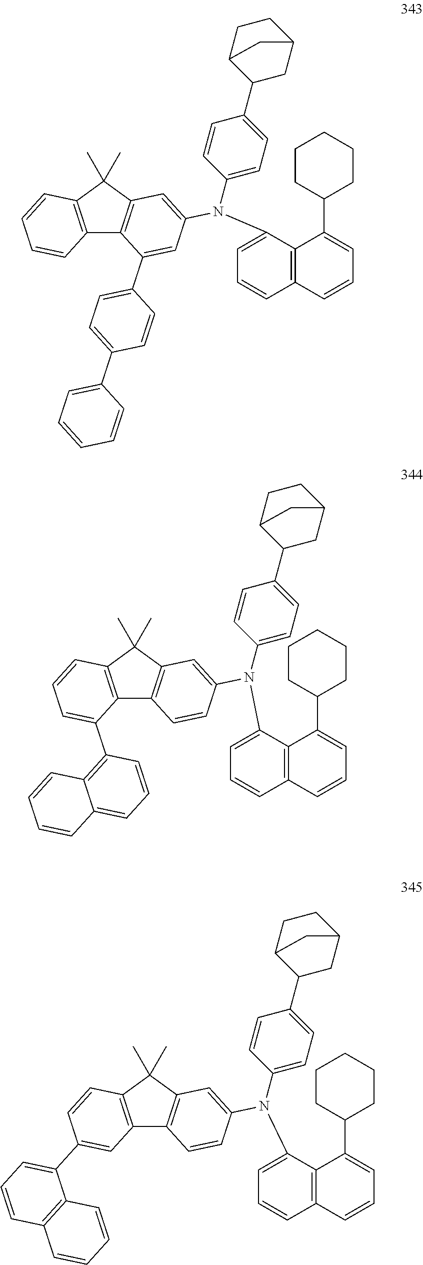 [SMILES: CC1(C)c2ccccc2-c2c(-c3ccc(-c4ccccc4)cc3)cc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cccc4cccc(C5CCCCC5)c34)cc21]

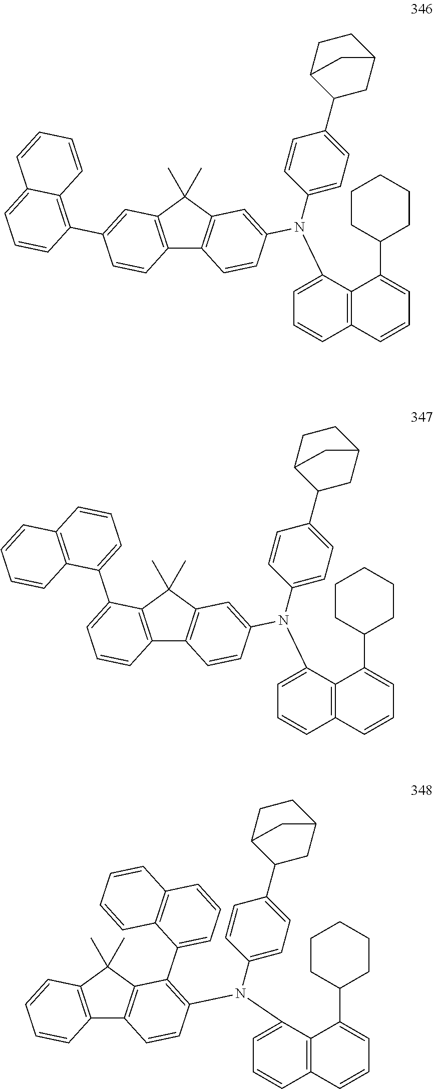 [SMILES: CC1(C)c2cc(-c3cccc4ccccc34)ccc2-c2ccc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)ccc2-c2cccc(-c3cccc4ccccc34)c21 ; CC1(C)c2ccccc2-c2ccc(N(c3ccc(C4CC5CCC4C5)cc3)c3cccc4cccc(C5CCCCC5)c34)c(-c3cccc4ccccc34)c21]

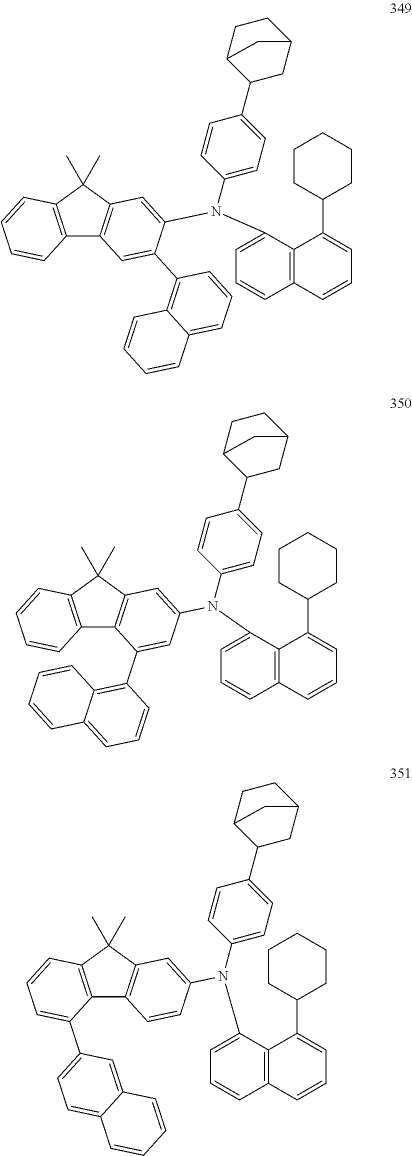 [SMILES: CC1(C)c2ccccc2-c2cc(-c3cccc4ccccc34)c(N(c3ccc(C4CC5CCC4C5)cc3)c3cccc4cccc(C5CCCCC5)c34)cc21 ; CC1(C)c2ccccc2-c2c(-c3cccc4ccccc34)cc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)ccc2-c2c(-c3ccc4ccccc4c3)cccc21]

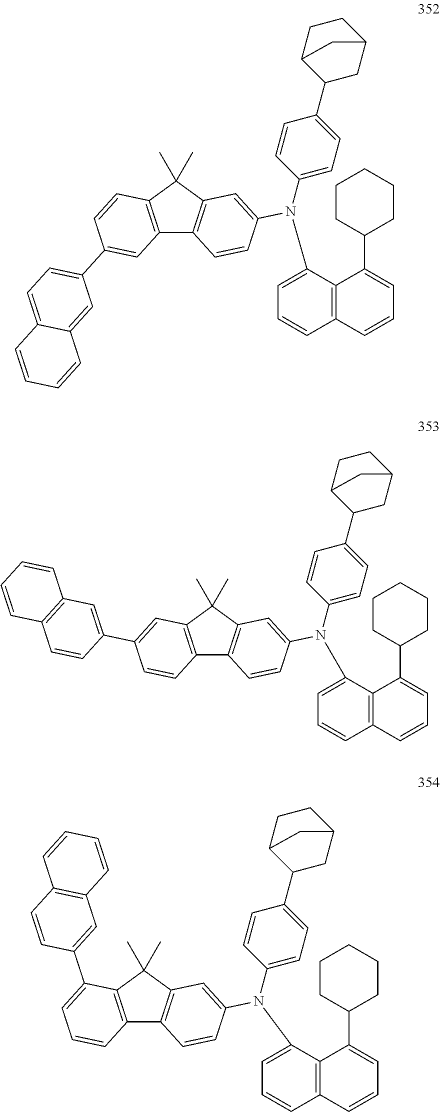 [SMILES: CC1(C)c2ccc(-c3ccc4ccccc4c3)cc2-c2ccc(N(c3ccc(C4CC5CCC4C5)cc3)c3cccc4cccc(C5CCCCC5)c34)cc21 ; CC1(C)c2cc(-c3ccc4ccccc4c3)ccc2-c2ccc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)ccc2-c2cccc(-c3ccc4ccccc4c3)c21]

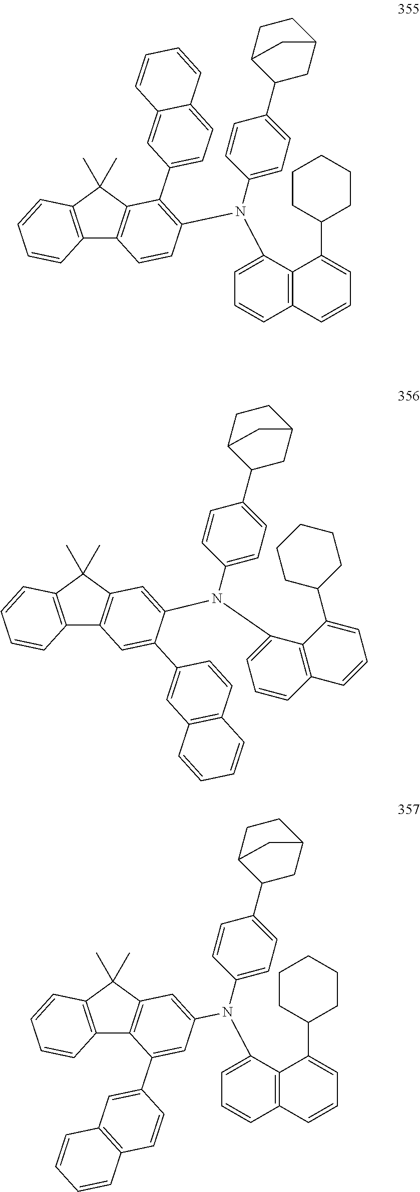 [SMILES: CC1(C)c2ccccc2-c2ccc(N(c3ccc(C4CC5CCC4C5)cc3)c3cccc4cccc(C5CCCCC5)c34)c(-c3ccc4ccccc4c3)c21 ; CC1(C)c2ccccc2-c2cc(-c3ccc4ccccc4c3)c(N(c3ccc(C4CC5CCC4C5)cc3)c3cccc4cccc(C5CCCCC5)c34)cc21 ; CC1(C)c2ccccc2-c2c(-c3ccc4ccccc4c3)cc(N(c3ccc(C4CC5CCC4C5)cc3)c3cccc4cccc(C5CCCCC5)c34)cc21]

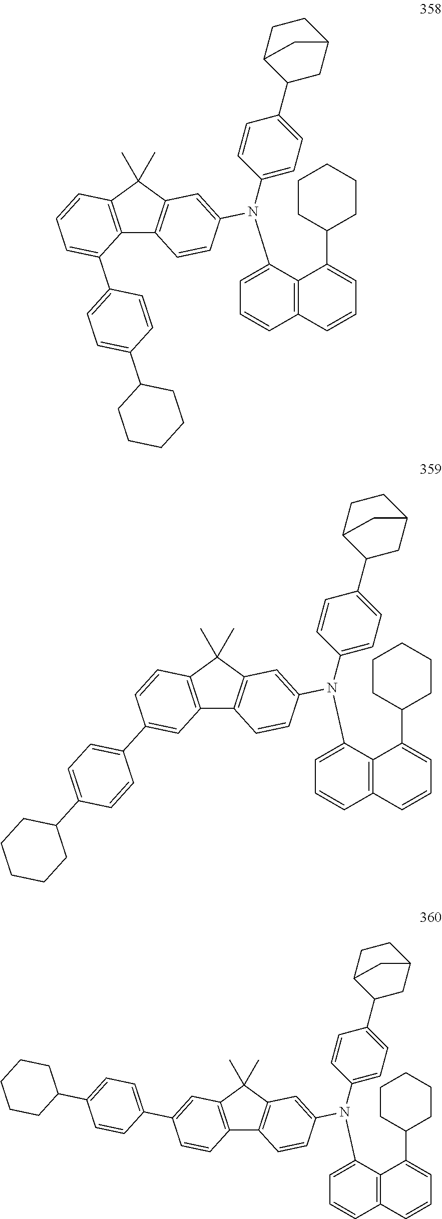 [SMILES: CC1(C)c2cc(N(c3ccc(C4CC5CCC4C5)cc3)c3cccc4cccc(C5CCCCC5)c34)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cccc4cccc(C5CCCCC5)c34)cc21 ; CC1(C)c2cc(-c3ccc(C4CCCCC4)cc3)ccc2-c2ccc(N(c3ccc(C4CC5CCC4C5)cc3)c3cccc4cccc(C5CCCCC5)c34)cc21]

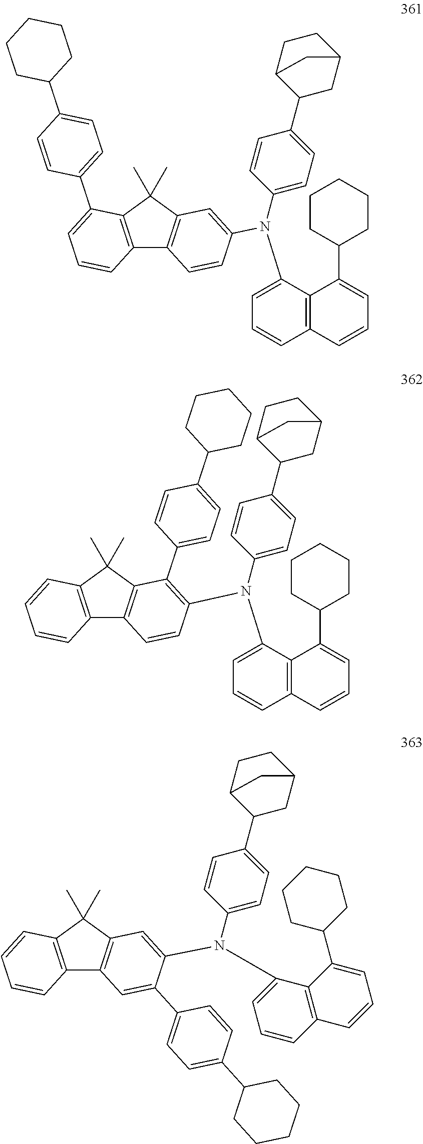

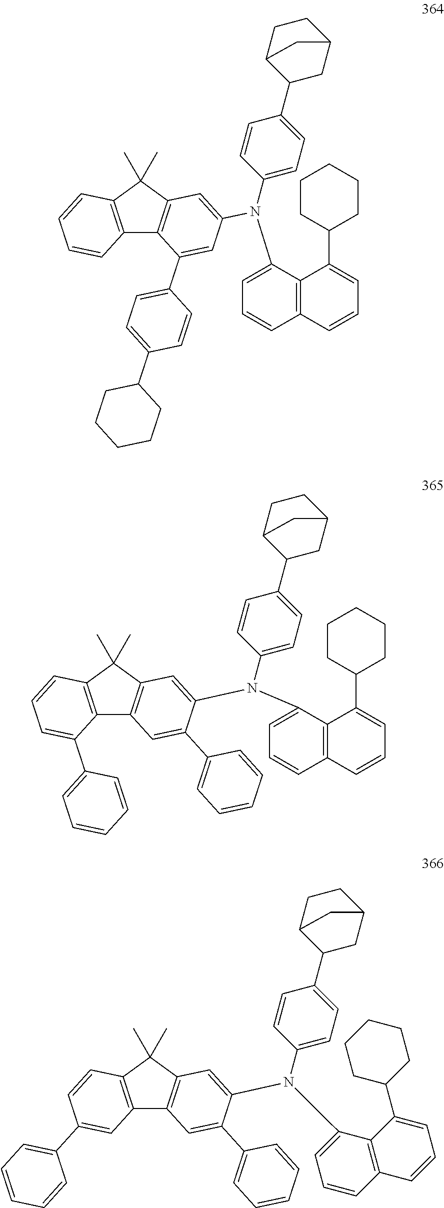 [SMILES: CC1(C)c2ccccc2-c2c(-c3ccc(C4CCCCC4)cc3)cc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)c(-c3ccccc3)cc2-c2c(-c3ccccc3)cccc21 ; CC1(C)c2ccc(-c3ccccc3)cc2-c2cc(-c3ccccc3)c(N(c3ccc(C4CC5CCC4C5)cc3)c3cccc4cccc(C5CCCCC5)c34)cc21]

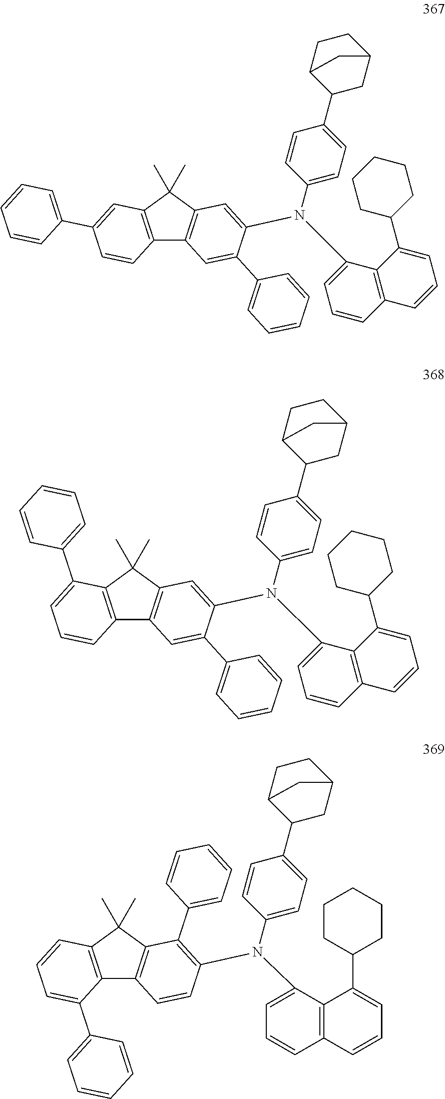 [SMILES: CC1(C)c2cc(-c3ccccc3)ccc2-c2cc(-c3ccccc3)c(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3cccc4cccc(C5CCCCC5)c34)c(-c3ccccc3)c21]

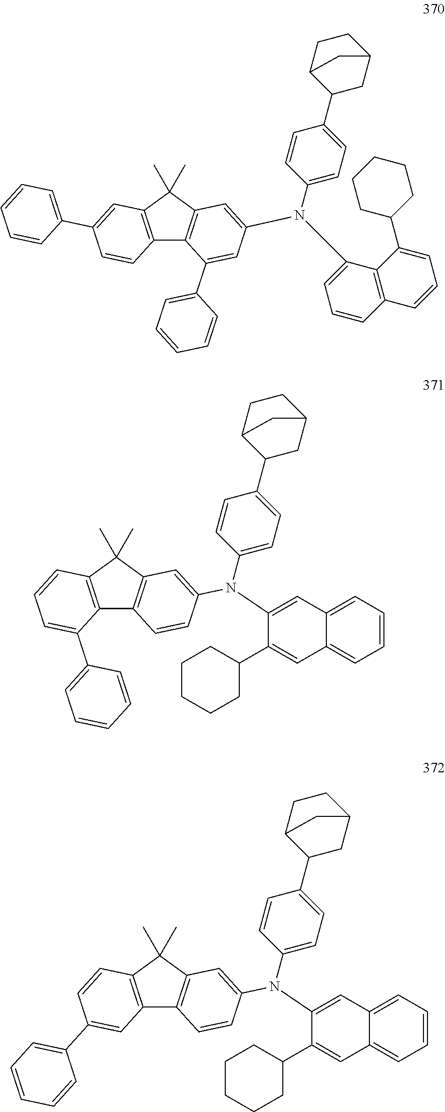 [SMILES: CC1(C)c2cc(-c3ccccc3)ccc2-c2c(-c3ccccc3)cc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cc4ccccc4cc3C3CCCCC3)ccc2-c2c(-c3ccccc3)cccc21 ; CC1(C)c2ccc(-c3ccccc3)cc2-c2ccc(N(c3ccc(C4CC5CCC4C5)cc3)c3cc4ccccc4cc3C3CCCCC3)cc21]

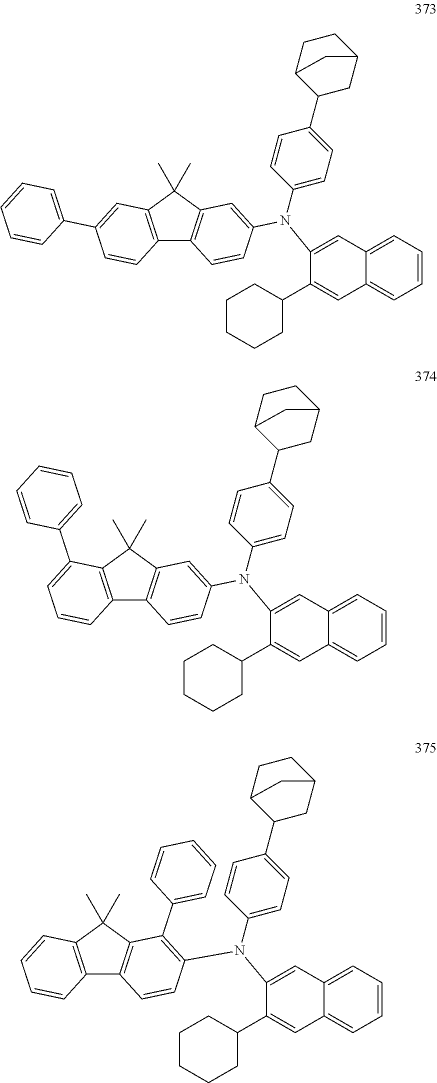 [SMILES: CC1(C)c2cc(-c3ccccc3)ccc2-c2ccc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)ccc2-c2cccc(-c3ccccc3)c21 ; CC1(C)c2ccccc2-c2ccc(N(c3ccc(C4CC5CCC4C5)cc3)c3cc4ccccc4cc3C3CCCCC3)c(-c3ccccc3)c21]

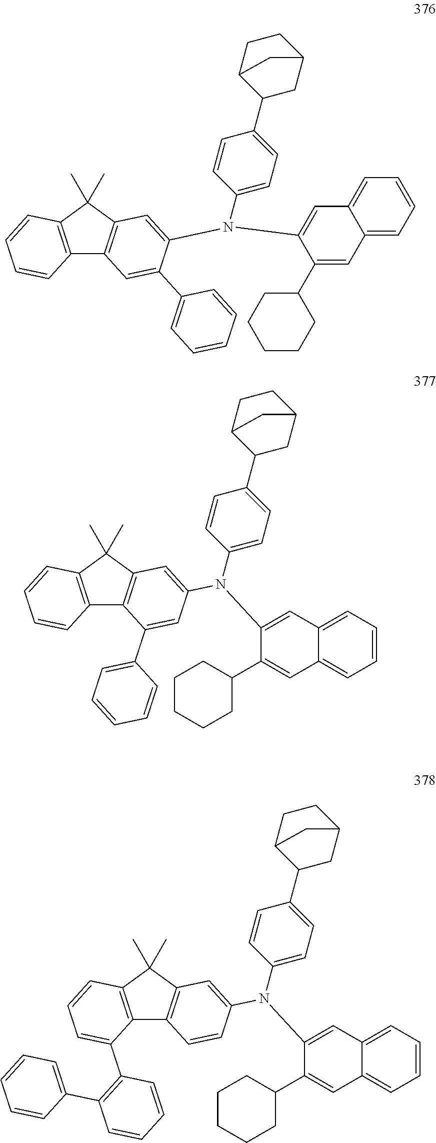

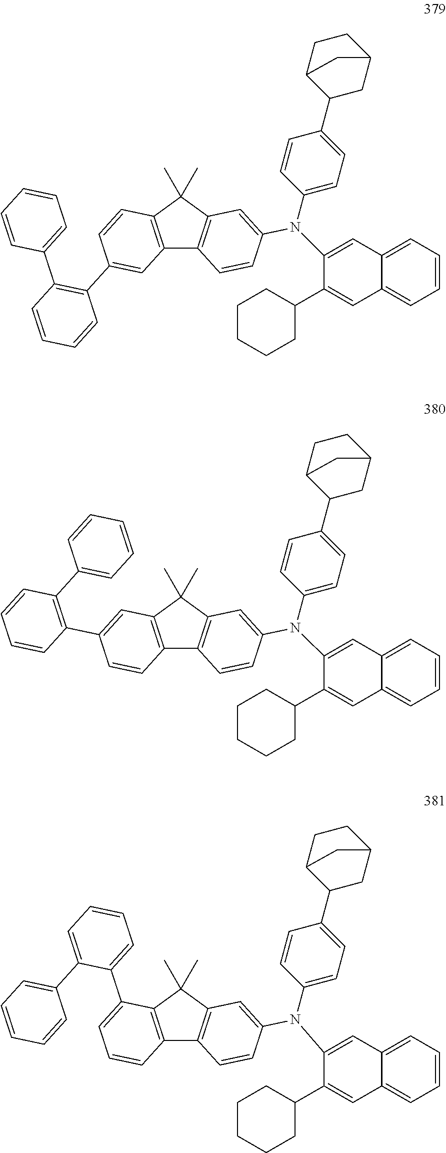 [SMILES: CC1(C)c2ccc(-c3ccccc3-c3ccccc3)cc2-c2ccc(N(c3ccc(C4CC5CCC4C5)cc3)c3cc4ccccc4cc3C3CCCCC3)cc21 ; CC1(C)c2cc(-c3ccccc3-c3ccccc3)ccc2-c2ccc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)ccc2-c2cccc(-c3ccccc3-c3ccccc3)c21]

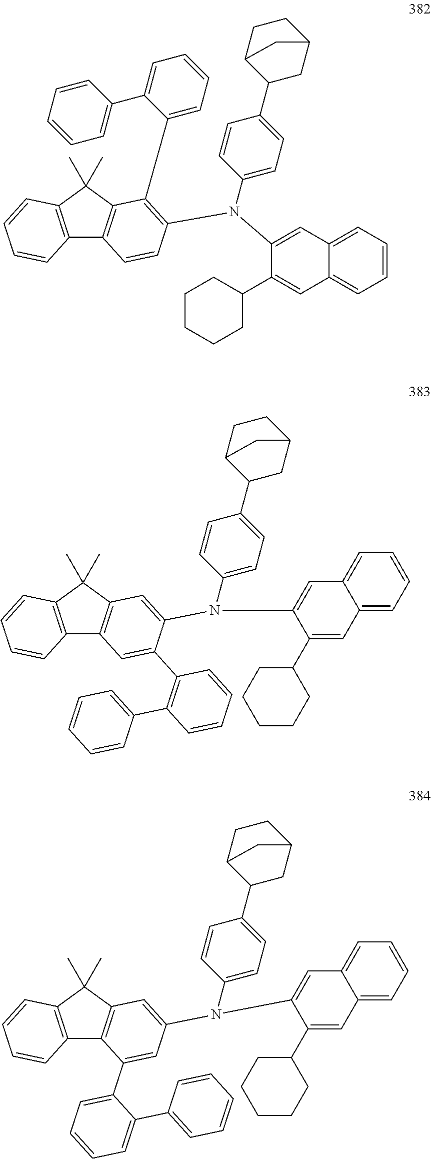 [SMILES: CC1(C)c2ccccc2-c2ccc(N(c3ccc(C4CC5CCC4C5)cc3)c3cc4ccccc4cc3C3CCCCC3)c(-c3ccccc3-c3ccccc3)c21 ; CC1(C)c2ccccc2-c2cc(-c3ccccc3-c3ccccc3)c(N(c3ccc(C4CC5CCC4C5)cc3)c3cc4ccccc4cc3C3CCCCC3)cc21 ; CC1(C)c2ccccc2-c2c(-c3ccccc3-c3ccccc3)cc(N(c3ccc(C4CC5CCC4C5)cc3)c3cc4ccccc4cc3C3CCCCC3)cc21]

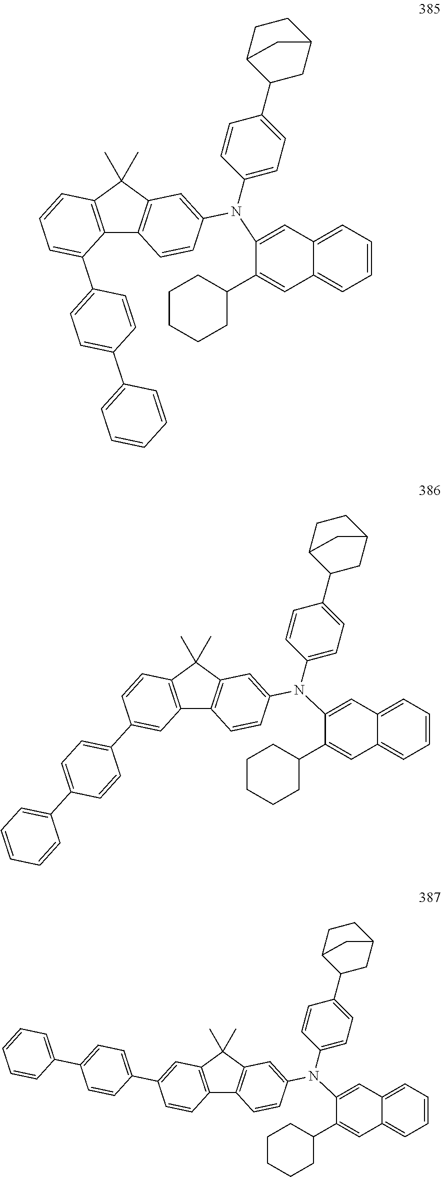 [SMILES: CC1(C)c2cc(N(c3ccc(C4CC5CCC4C5)cc3)c3cc4ccccc4cc3C3CCCCC3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3cc4ccccc4cc3C3CCCCC3)cc21 ; CC1(C)c2cc(-c3ccc(-c4ccccc4)cc3)ccc2-c2ccc(N(c3ccc(C4CC5CCC4C5)cc3)c3cc4ccccc4cc3C3CCCCC3)cc21]

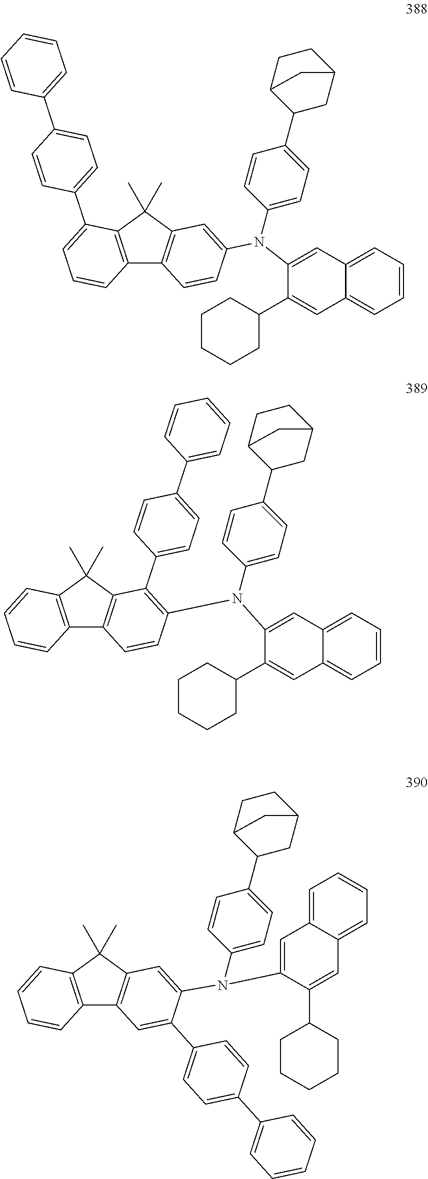

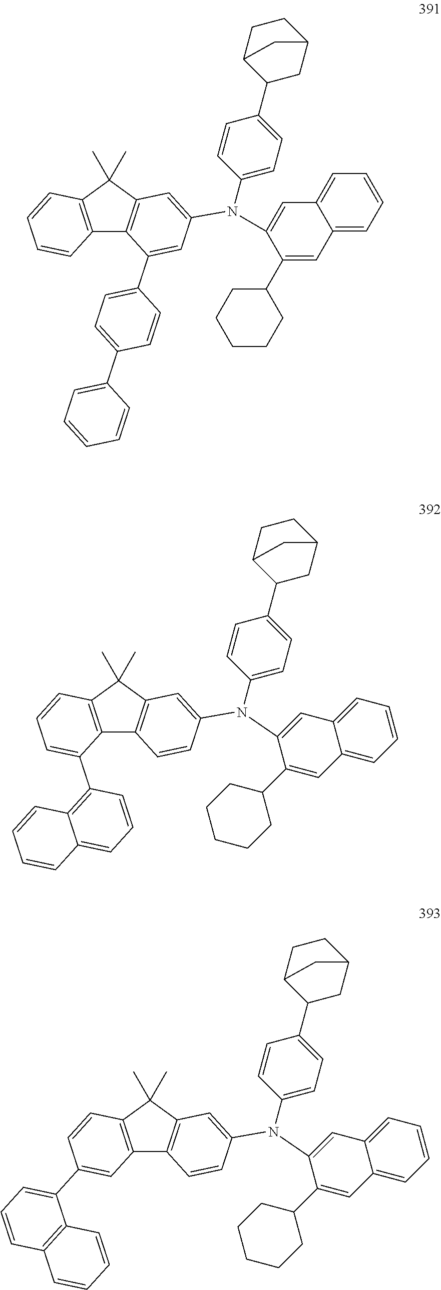 [SMILES: CC1(C)c2ccccc2-c2c(-c3ccc(-c4ccccc4)cc3)cc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cc4ccccc4cc3C3CCCCC3)cc21]

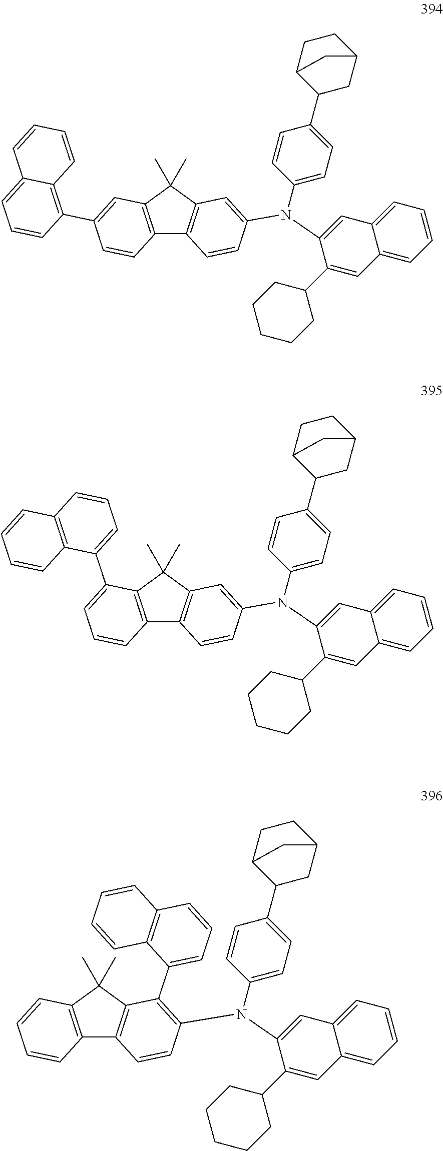 [SMILES: CC1(C)c2cc(-c3cccc4ccccc34)ccc2-c2ccc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)ccc2-c2cccc(-c3cccc4ccccc34)c21 ; CC1(C)c2ccccc2-c2ccc(N(c3ccc(C4CC5CCC4C5)cc3)c3cc4ccccc4cc3C3CCCCC3)c(-c3cccc4ccccc34)c21]

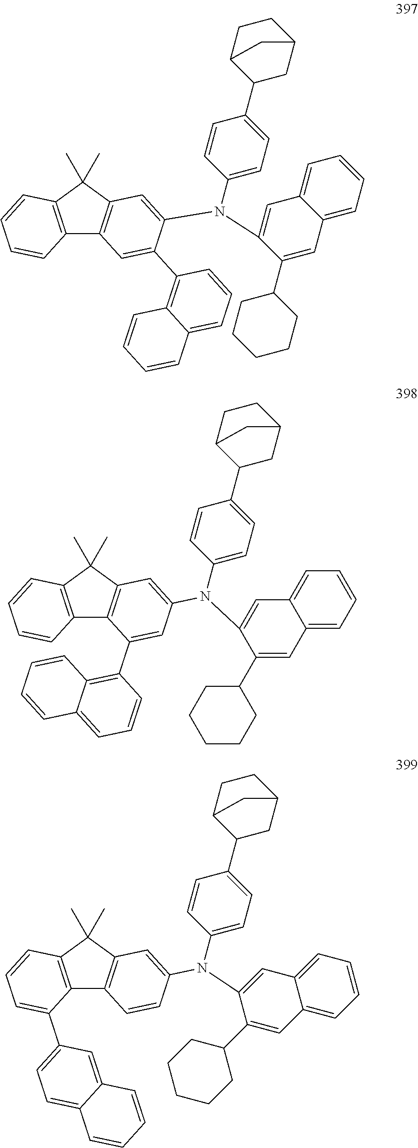

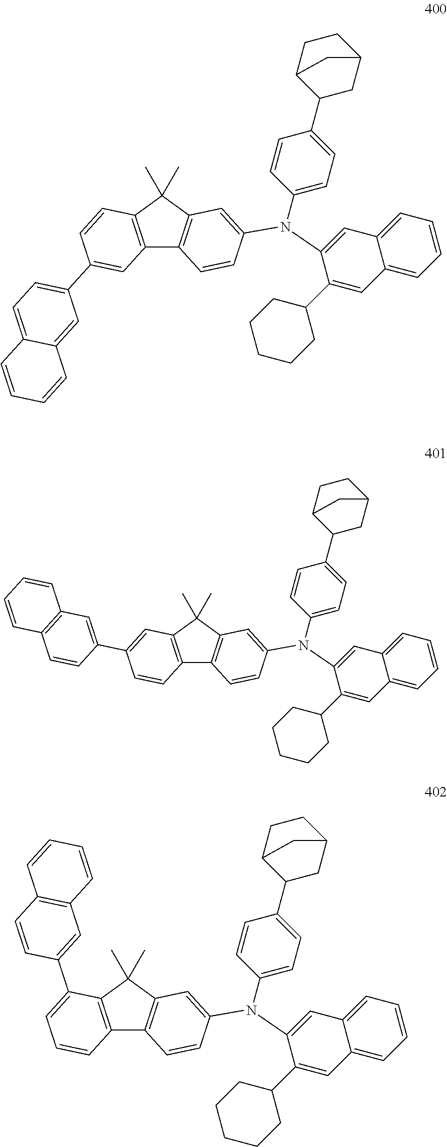 [SMILES: CC1(C)c2ccc(-c3ccc4ccccc4c3)cc2-c2ccc(N(c3ccc(C4CC5CCC4C5)cc3)c3cc4ccccc4cc3C3CCCCC3)cc21 ; CC1(C)c2cc(-c3ccc4ccccc4c3)ccc2-c2ccc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)ccc2-c2cccc(-c3ccc4ccccc4c3)c21]

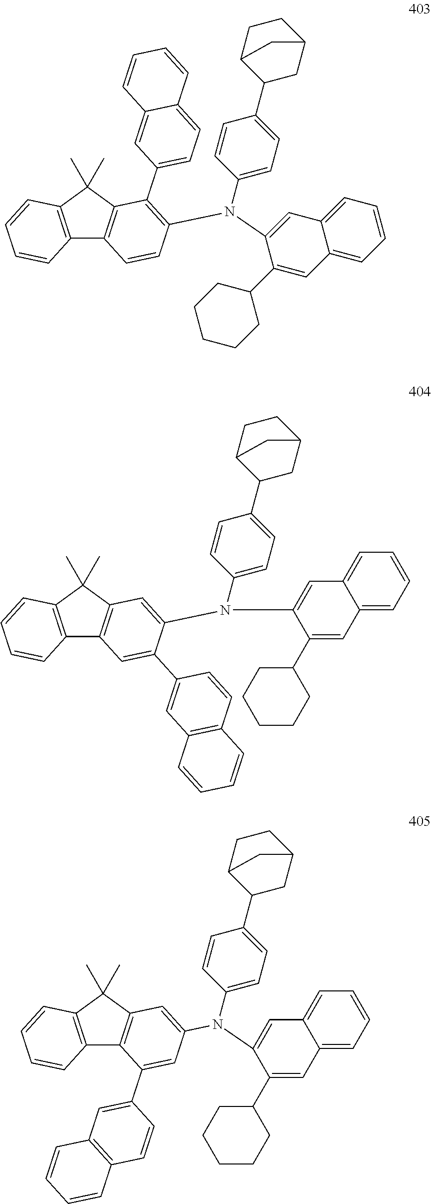 [SMILES: CC1(C)c2ccccc2-c2ccc(N(c3ccc(C4CC5CCC4C5)cc3)c3cc4ccccc4cc3C3CCCCC3)c(-c3ccc4ccccc4c3)c21 ; CC1(C)c2ccccc2-c2cc(-c3ccc4ccccc4c3)c(N(c3ccc(C4CC5CCC4C5)cc3)c3cc4ccccc4cc3C3CCCCC3)cc21 ; CC1(C)c2ccccc2-c2c(-c3ccc4ccccc4c3)cc(N(c3ccc(C4CC5CCC4C5)cc3)c3cc4ccccc4cc3C3CCCCC3)cc21]

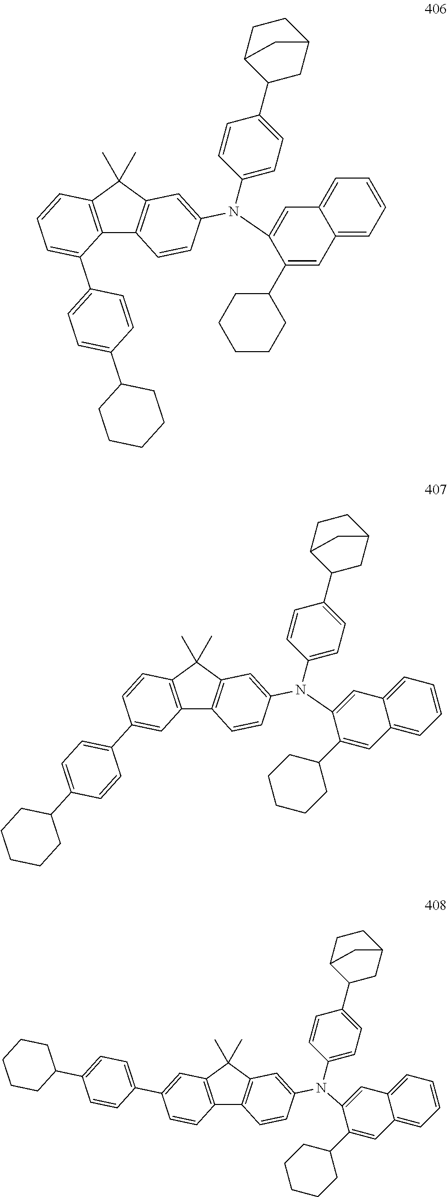 [SMILES: CC1(C)c2cc(N(c3ccc(C4CC5CCC4C5)cc3)c3cc4ccccc4cc3C3CCCCC3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cc4ccccc4cc3C3CCCCC3)cc21 ; CC1(C)c2cc(-c3ccc(C4CCCCC4)cc3)ccc2-c2ccc(N(c3ccc(C4CC5CCC4C5)cc3)c3cc4ccccc4cc3C3CCCCC3)cc21]

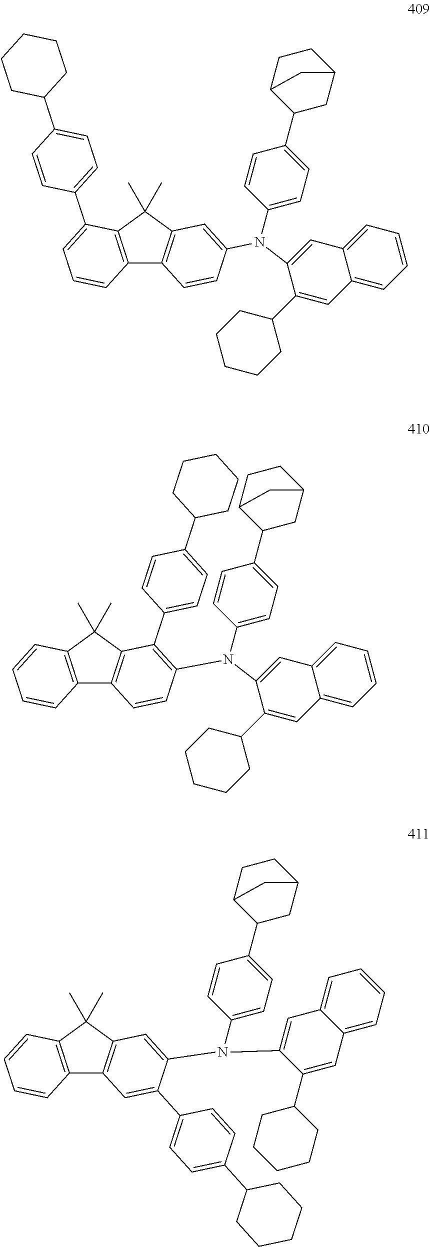 [SMILES: CC1(C)c2cc(N(c3ccc(C4CC5CCC4C5)cc3)c3cc4ccccc4cc3C3CCCCC3)ccc2-c2cccc(-c3ccc(C4CCCCC4)cc3)c21 ; CC1(C)c2ccccc2-c2ccc(N(c3ccc(C4CC5CCC4C5)cc3)c3cc4ccccc4cc3C3CCCCC3)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3cc4ccccc4cc3C3CCCCC3)cc21]

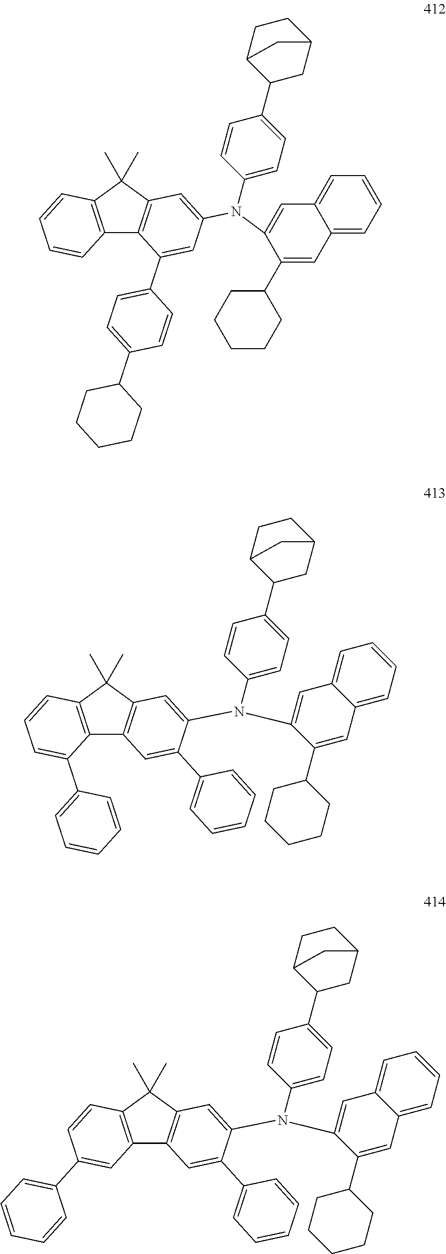 [SMILES: CC1(C)c2ccccc2-c2c(-c3ccc(C4CCCCC4)cc3)cc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)c(-c3ccccc3)cc2-c2c(-c3ccccc3)cccc21 ; CC1(C)c2ccc(-c3ccccc3)cc2-c2cc(-c3ccccc3)c(N(c3ccc(C4CC5CCC4C5)cc3)c3cc4ccccc4cc3C3CCCCC3)cc21]

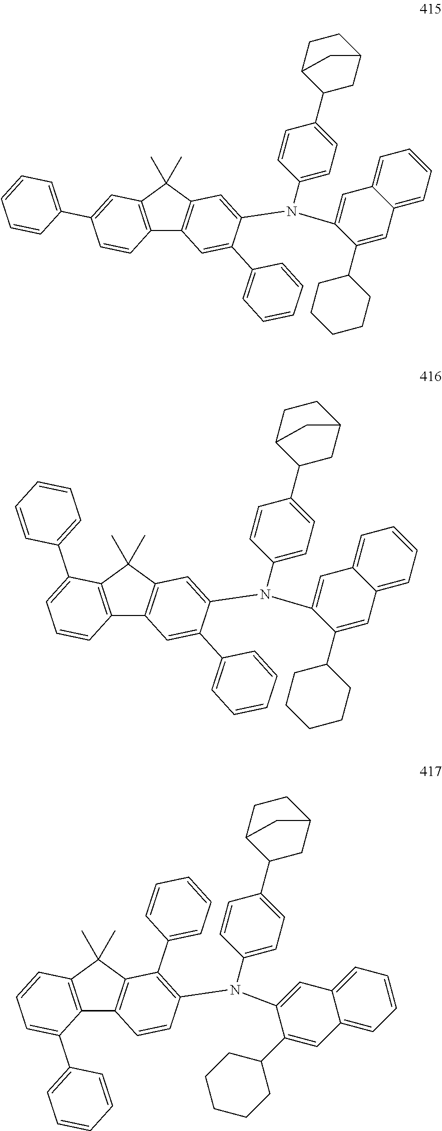 [SMILES: CC1(C)c2cc(-c3ccccc3)ccc2-c2cc(-c3ccccc3)c(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3cc4ccccc4cc3C3CCCCC3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3cc4ccccc4cc3C3CCCCC3)c(-c3ccccc3)c21]

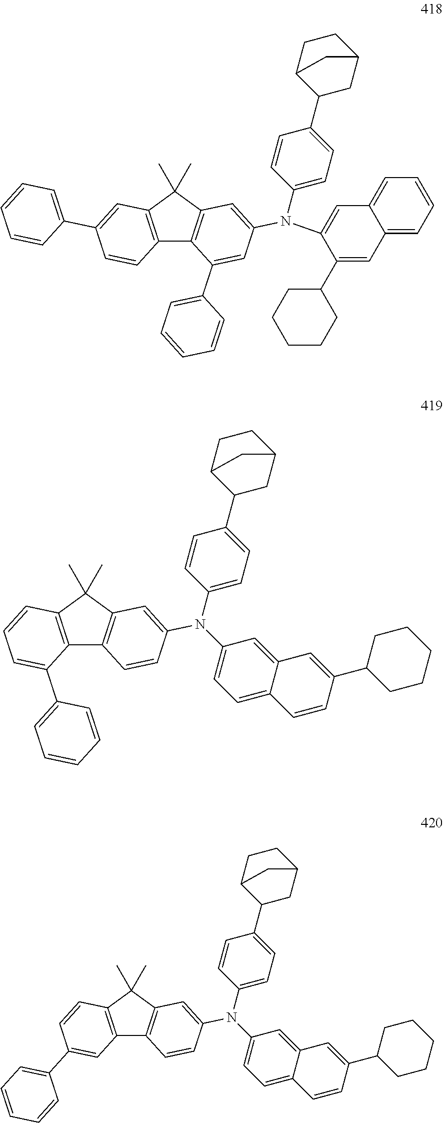 [SMILES: CC1(C)c2cc(-c3ccccc3)ccc2-c2c(-c3ccccc3)cc(N(c3ccc(C4CC5CCC4C5)cc3)c3cc4ccccc4cc3C3CCCCC3)cc21 ; CC1(C)c2cc(N(c3ccc(C4CC5CCC4C5)cc3)c3ccc4ccc(C5CCCCC5)cc4c3)ccc2-c2c(-c3ccccc3)cccc21 ; CC1(C)c2ccc(-c3ccccc3)cc2-c2ccc(N(c3ccc(C4CC5CCC4C5)cc3)c3ccc4ccc(C5CCCCC5)cc4c3)cc21]

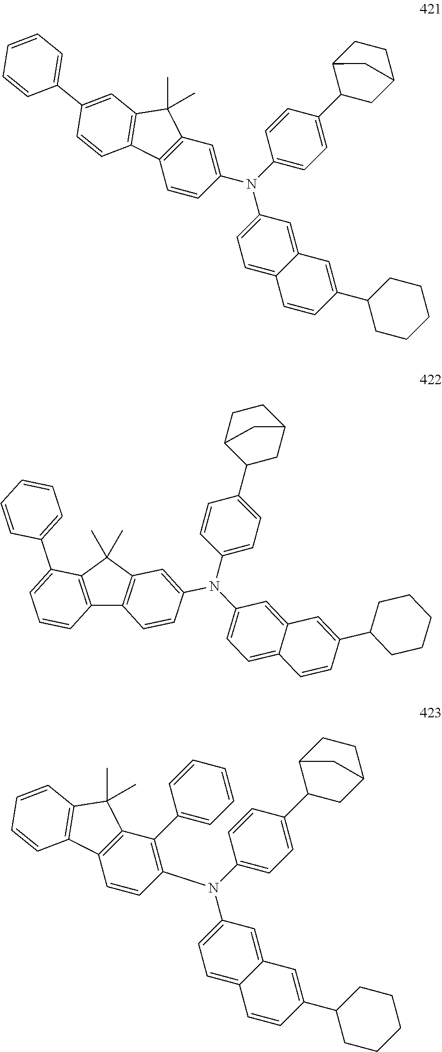 [SMILES: CC1(C)c2cc(-c3ccccc3)ccc2-c2ccc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2cccc(-c3ccccc3)c21 ; CC1(C)c2ccccc2-c2ccc(N(c3ccc(C4CC5CCC4C5)cc3)c3ccc4ccc(C5CCCCC5)cc4c3)c(-c3ccccc3)c21]

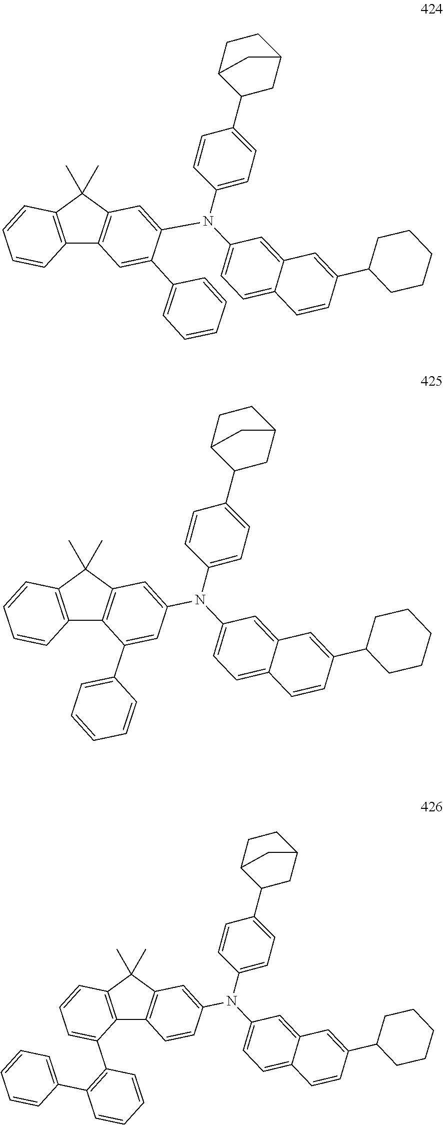 [SMILES: CC1(C)c2ccccc2-c2cc(-c3ccccc3)c(N(c3ccc(C4CC5CCC4C5)cc3)c3ccc4ccc(C5CCCCC5)cc4c3)cc21 ; CC1(C)c2ccccc2-c2c(-c3ccccc3)cc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2c(-c3ccccc3-c3ccccc3)cccc21]

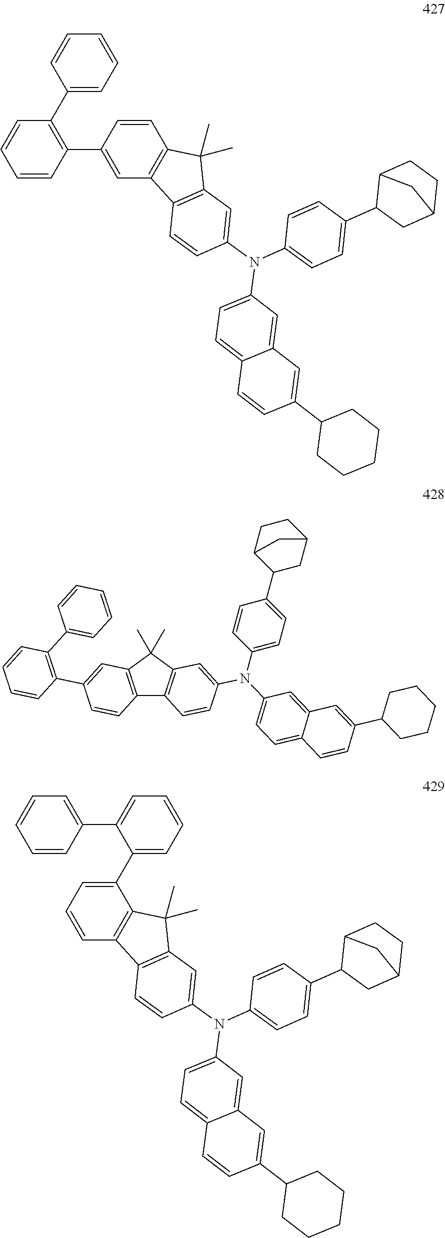 [SMILES: CC1(C)c2ccc(-c3ccccc3-c3ccccc3)cc2-c2ccc(N(c3ccc(C4CC5CCC4C5)cc3)c3ccc4ccc(C5CCCCC5)cc4c3)cc21 ; CC1(C)c2cc(-c3ccccc3-c3ccccc3)ccc2-c2ccc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2cccc(-c3ccccc3-c3ccccc3)c21]

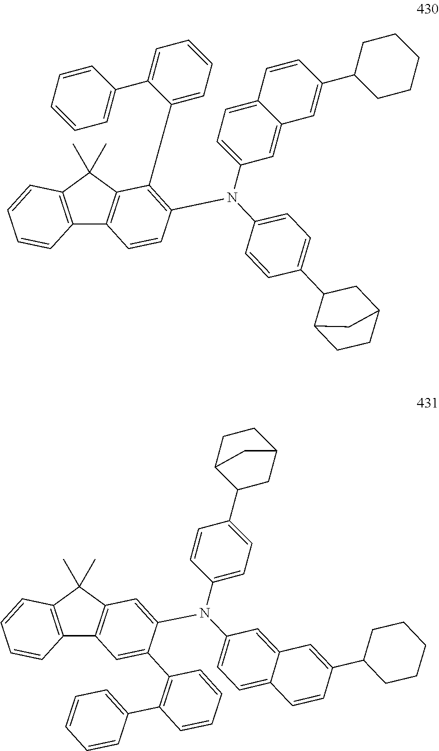 [SMILES: CC1(C)c2ccccc2-c2ccc(N(c3ccc(C4CC5CCC4C5)cc3)c3ccc4ccc(C5CCCCC5)cc4c3)c(-c3ccccc3-c3ccccc3)c21 ; CC1(C)c2ccccc2-c2cc(-c3ccccc3-c3ccccc3)c(N(c3ccc(C4CC5CCC4C5)cc3)c3ccc4ccc(C5CCCCC5)cc4c3)cc21]

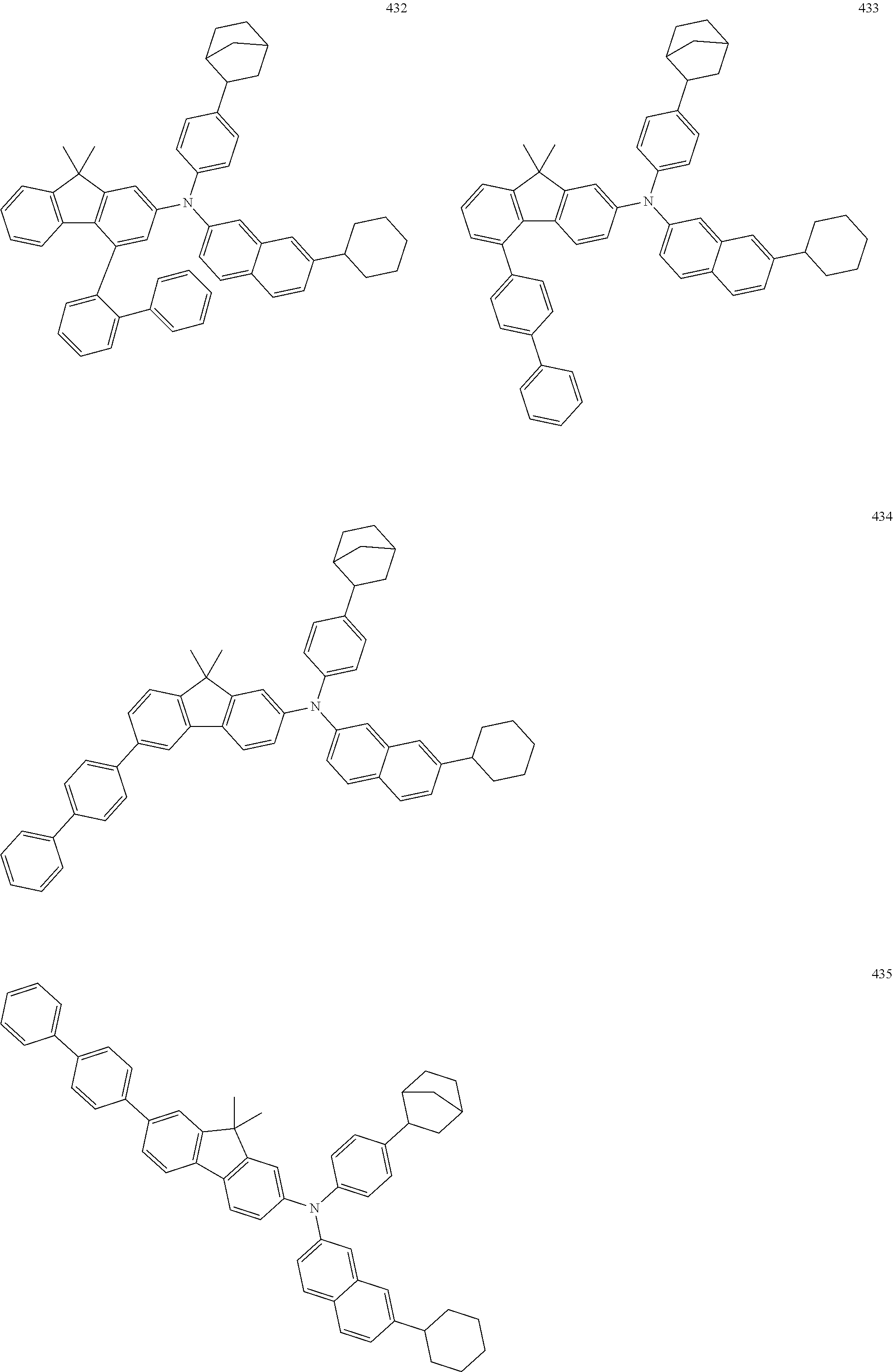 [SMILES: CC1(C)c2ccccc2-c2c(-c3ccccc3-c3ccccc3)cc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3ccc4ccc(C5CCCCC5)cc4c3)cc21 ; CC1(C)c2cc(-c3ccc(-c4ccccc4)cc3)ccc2-c2ccc(N(c3ccc(C4CC5CCC4C5)cc3)c3ccc4ccc(C5CCCCC5)cc4c3)cc21]

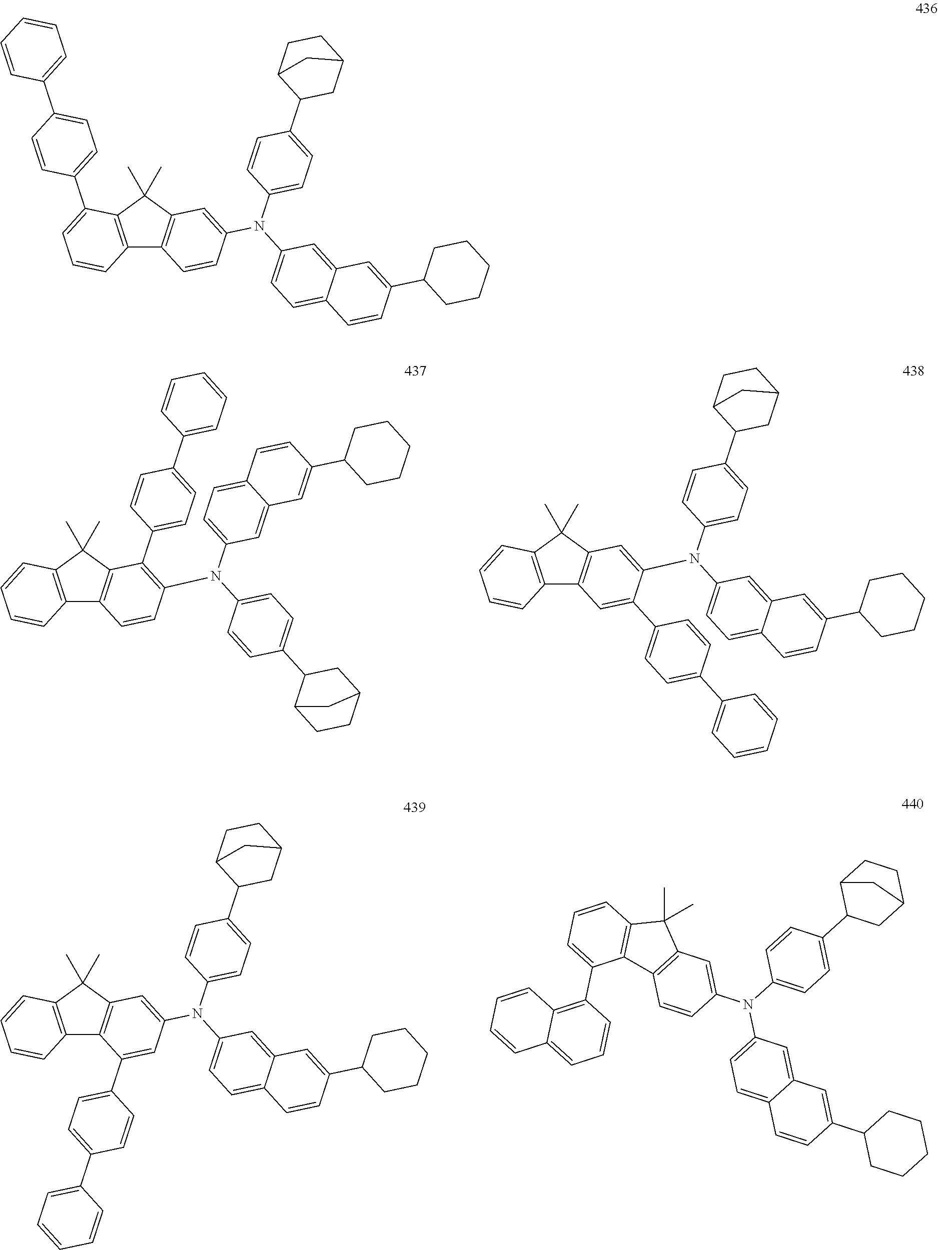 [SMILES: CC1(C)c2cc(N(c3ccc(C4CC5CCC4C5)cc3)c3ccc4ccc(C5CCCCC5)cc4c3)ccc2-c2cccc(-c3ccc(-c4ccccc4)cc3)c21 ; CC1(C)c2ccccc2-c2ccc(N(c3ccc(C4CC5CCC4C5)cc3)c3ccc4ccc(C5CCCCC5)cc4c3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3ccc4ccc(C5CCCCC5)cc4c3)cc21 ; CC1(C)c2ccccc2-c2c(-c3ccc(-c4ccccc4)cc3)cc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2c(-c3cccc4ccccc34)cccc21]

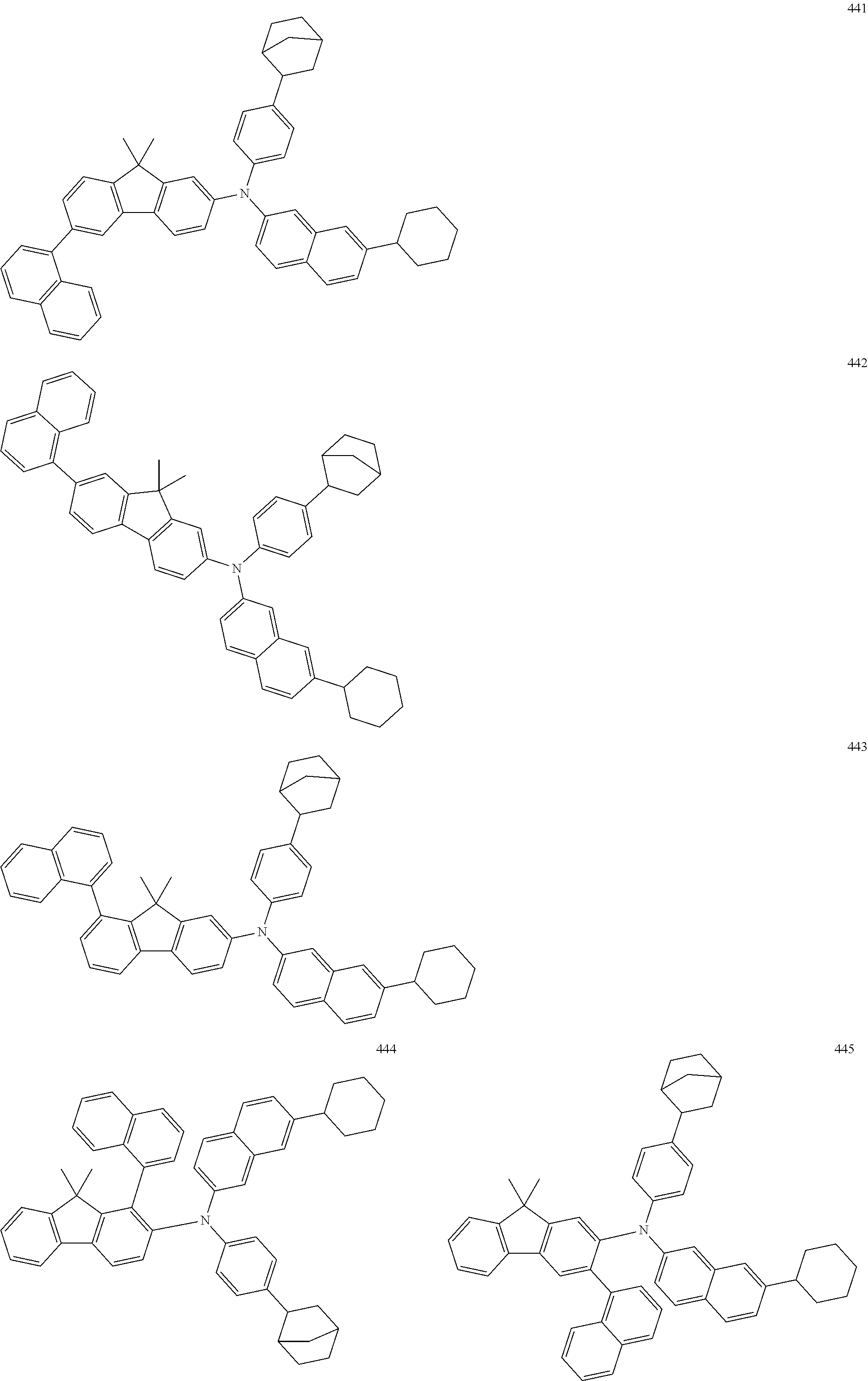

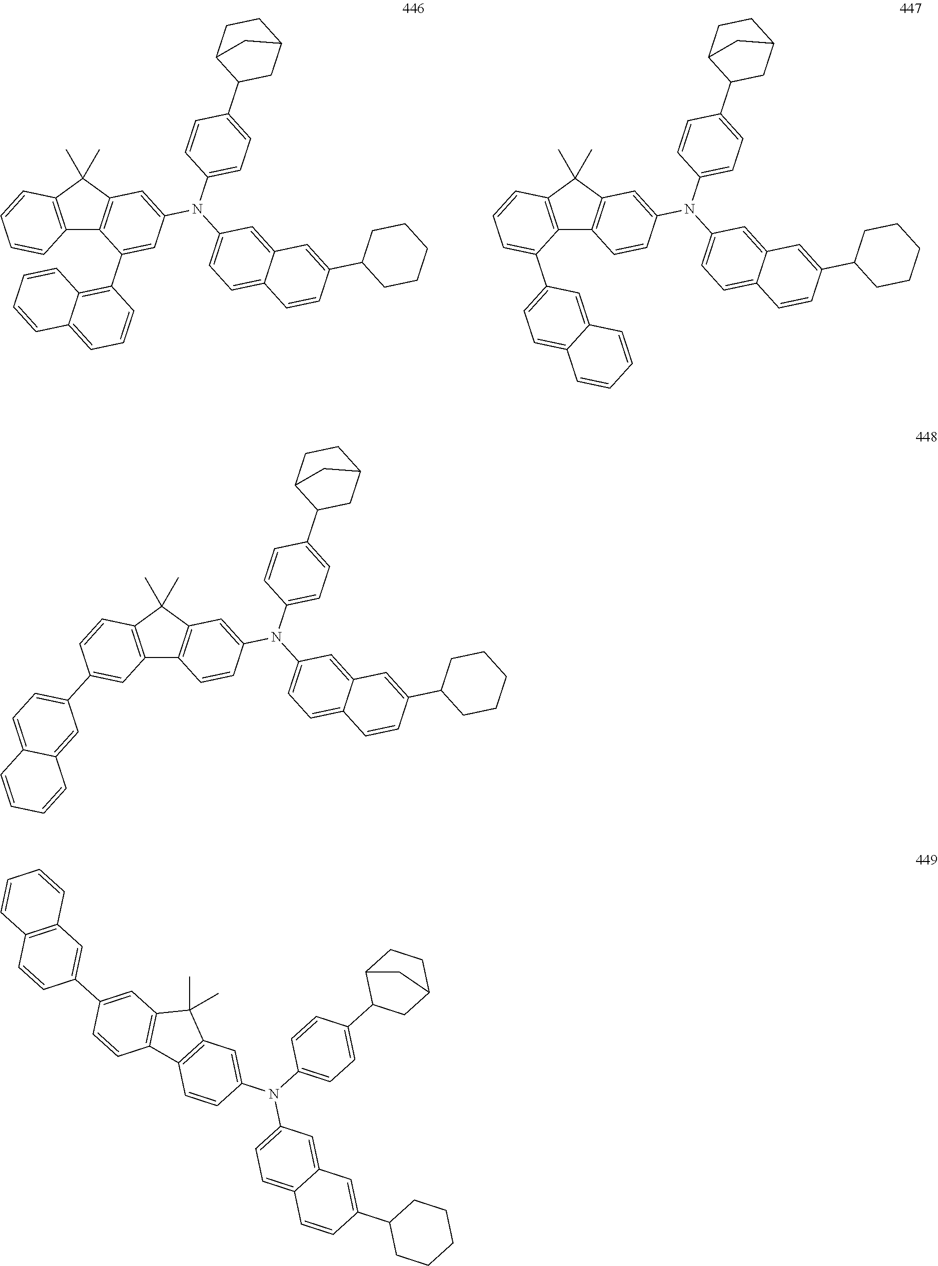 [SMILES: CC1(C)c2ccccc2-c2c(-c3cccc4ccccc34)cc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccc4ccc(C5CCCCC5)cc4c3)cc21 ; CC1(C)c2cc(-c3ccc4ccccc4c3)ccc2-c2ccc(N(c3ccc(C4CC5CCC4C5)cc3)c3ccc4ccc(C5CCCCC5)cc4c3)cc21]

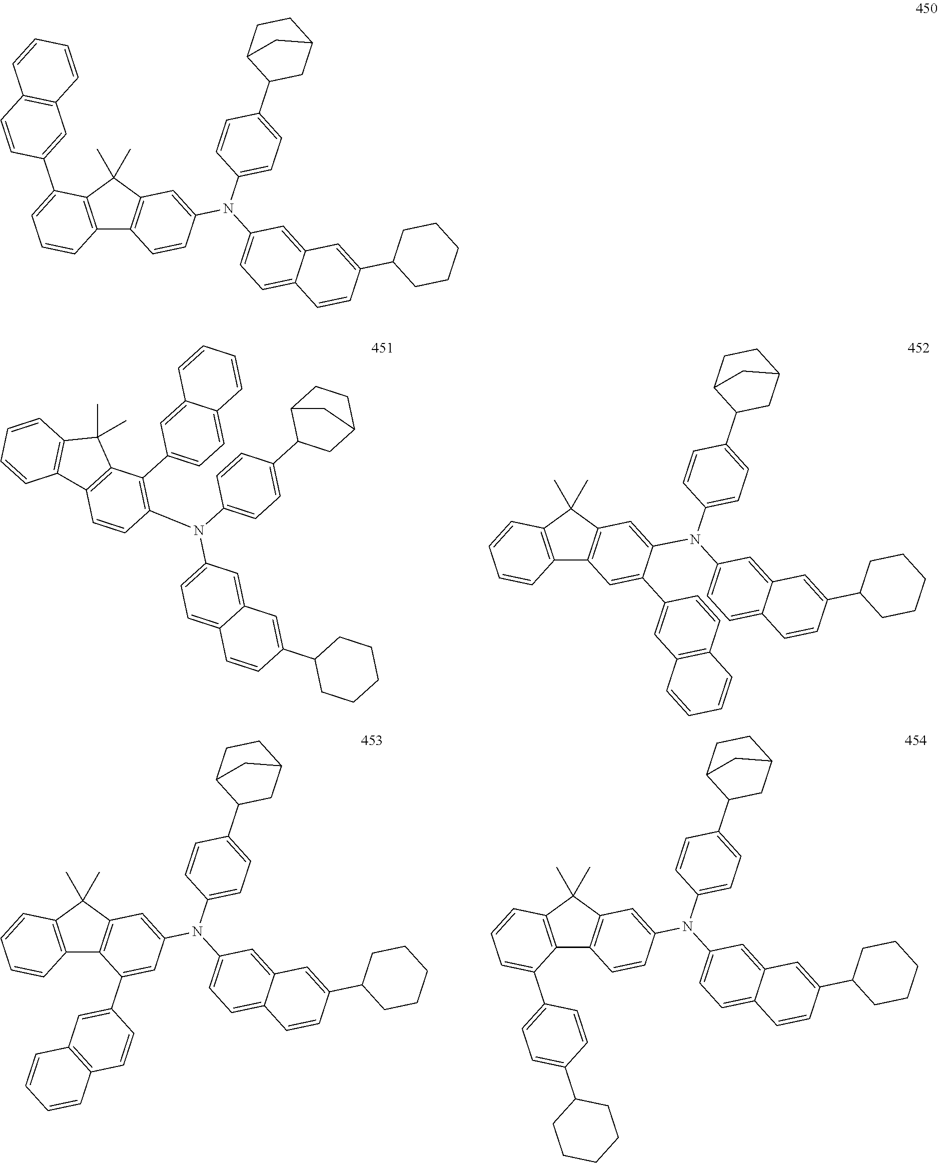

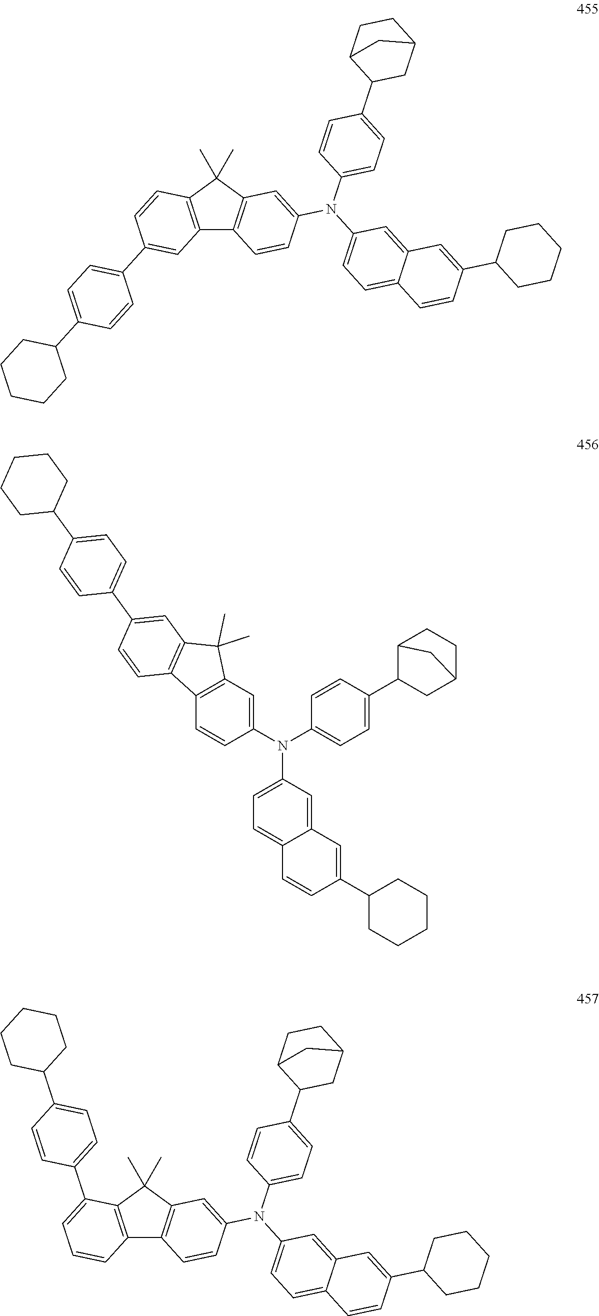 [SMILES: CC1(C)c2ccc(-c3ccc(C4CCCCC4)cc3)cc2-c2ccc(N(c3ccc(C4CC5CCC4C5)cc3)c3ccc4ccc(C5CCCCC5)cc4c3)cc21 ; CC1(C)c2cc(-c3ccc(C4CCCCC4)cc3)ccc2-c2ccc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2cccc(-c3ccc(C4CCCCC4)cc3)c21]

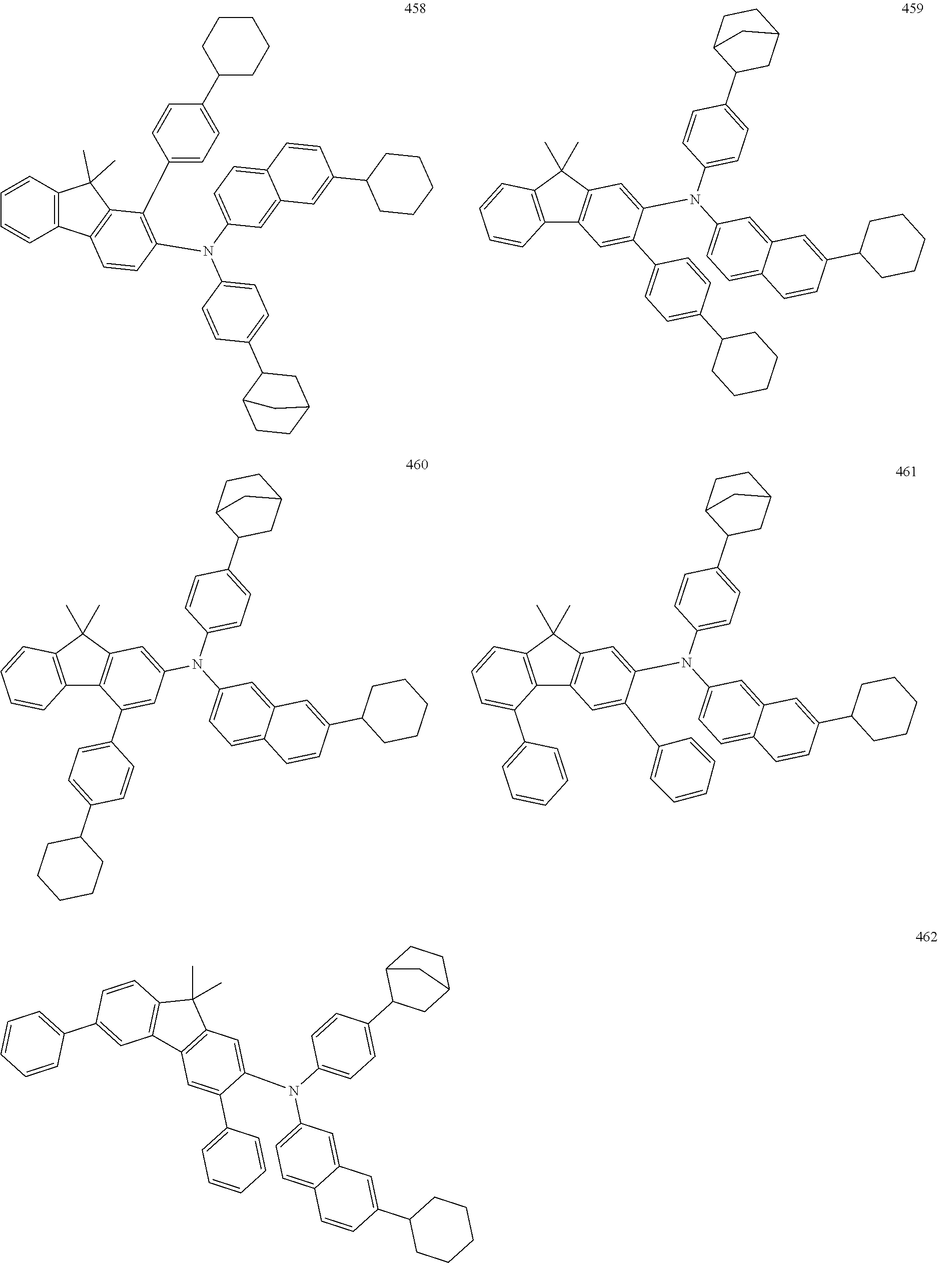

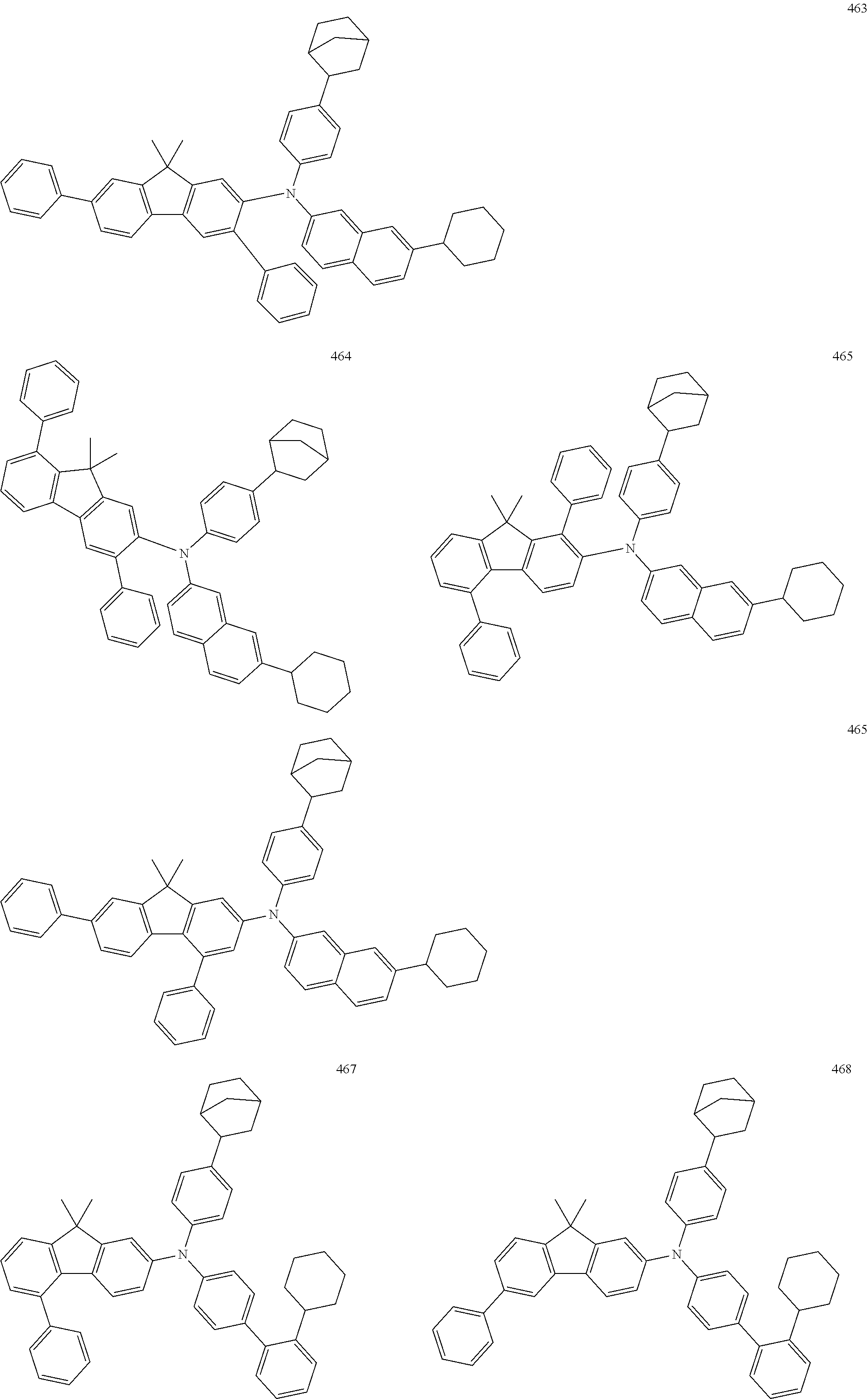

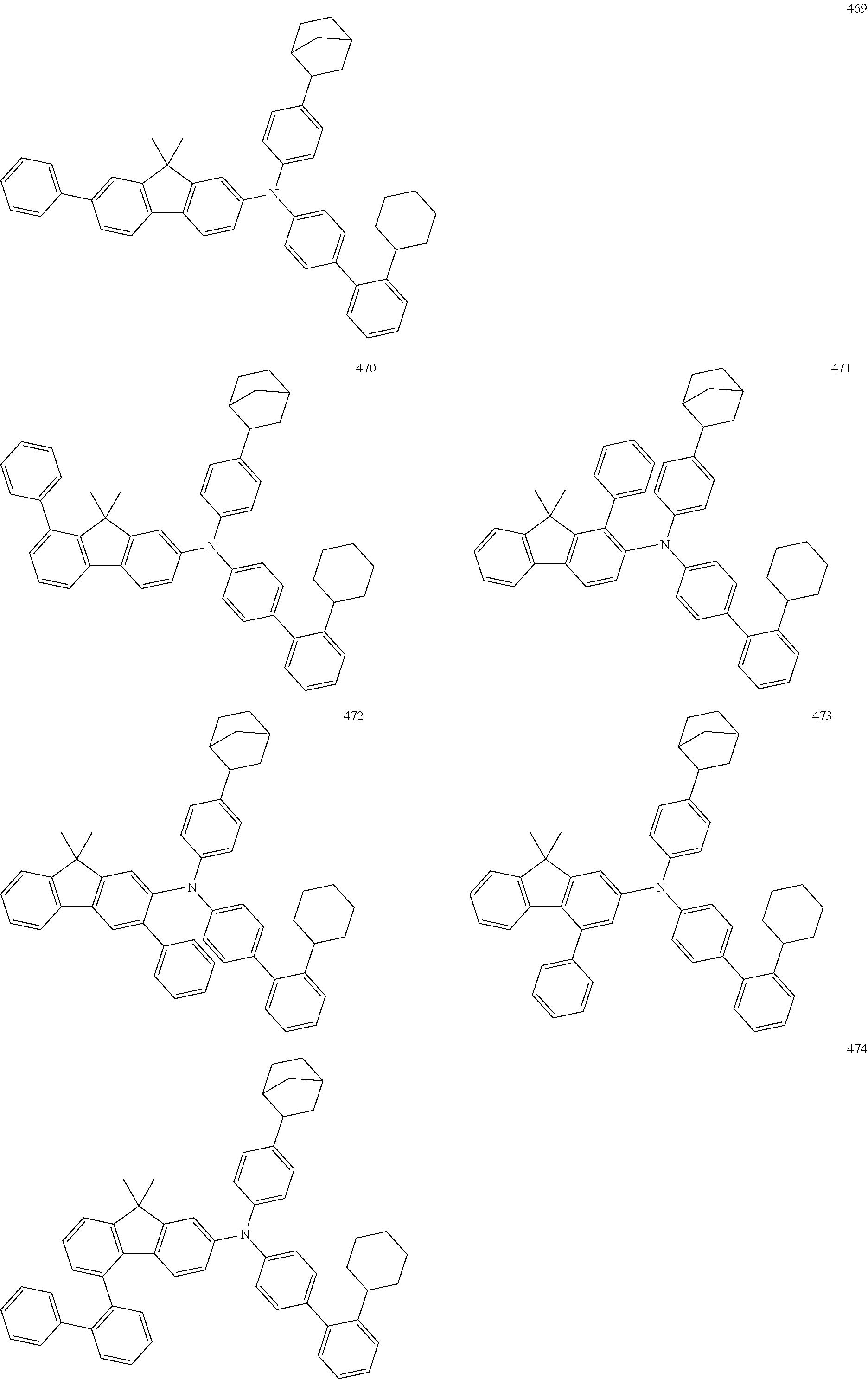

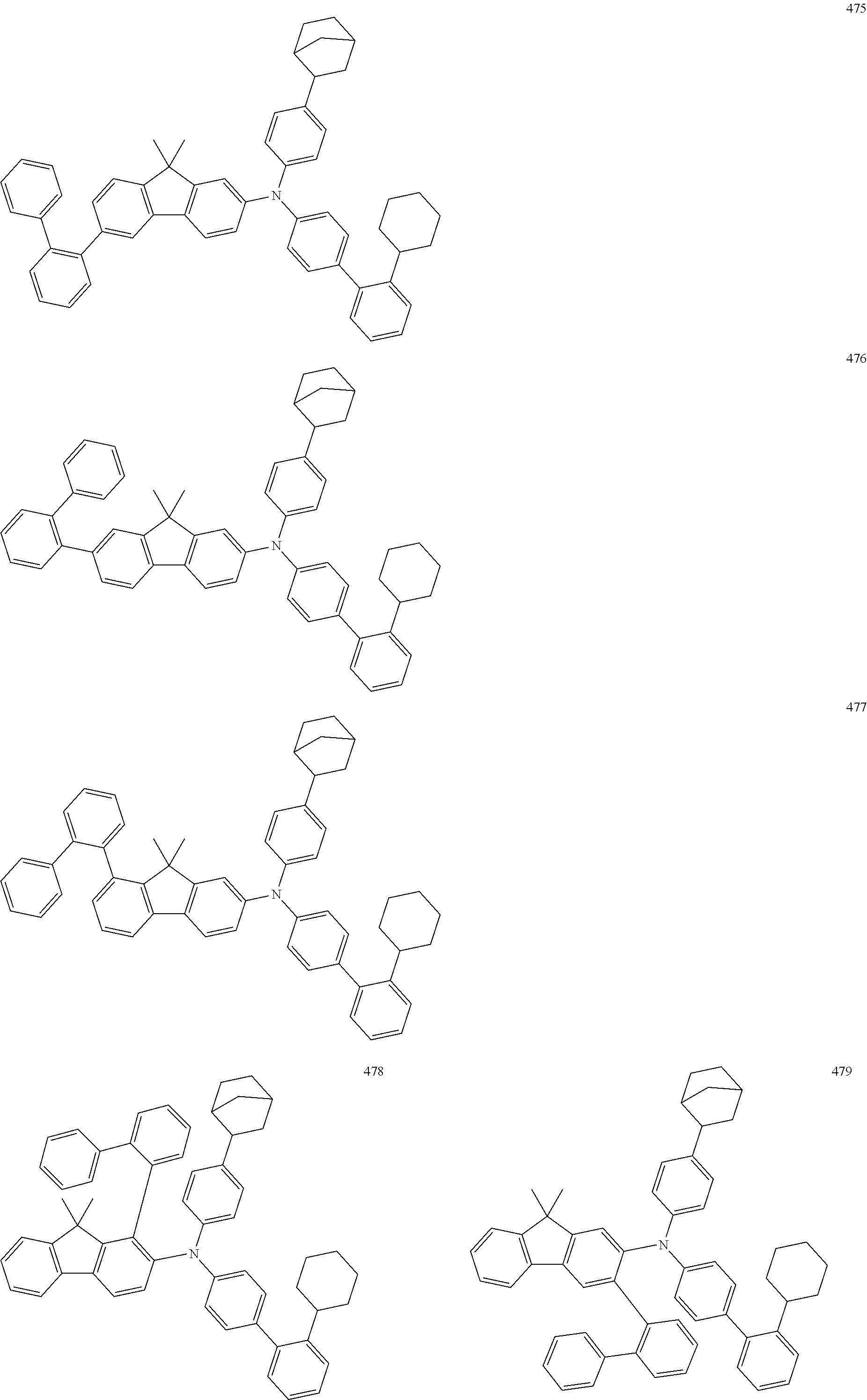 [SMILES: CC1(C)c2ccc(-c3ccccc3-c3ccccc3)cc2-c2ccc(N(c3ccc(-c4ccccc4C4CCCCC4)cc3)c3ccc(C4CC5CCC4C5)cc3)cc21 ; CC1(C)c2cc(-c3ccccc3-c3ccccc3)ccc2-c2ccc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2cccc(-c3ccccc3-c3ccccc3)c21 ; CC1(C)c2ccccc2-c2ccc(N(c3ccc(-c4ccccc4C4CCCCC4)cc3)c3ccc(C4CC5CCC4C5)cc3)c(-c3ccccc3-c3ccccc3)c21 ; CC1(C)c2ccccc2-c2cc(-c3ccccc3-c3ccccc3)c(N(c3ccc(-c4ccccc4C4CCCCC4)cc3)c3ccc(C4CC5CCC4C5)cc3)cc21]

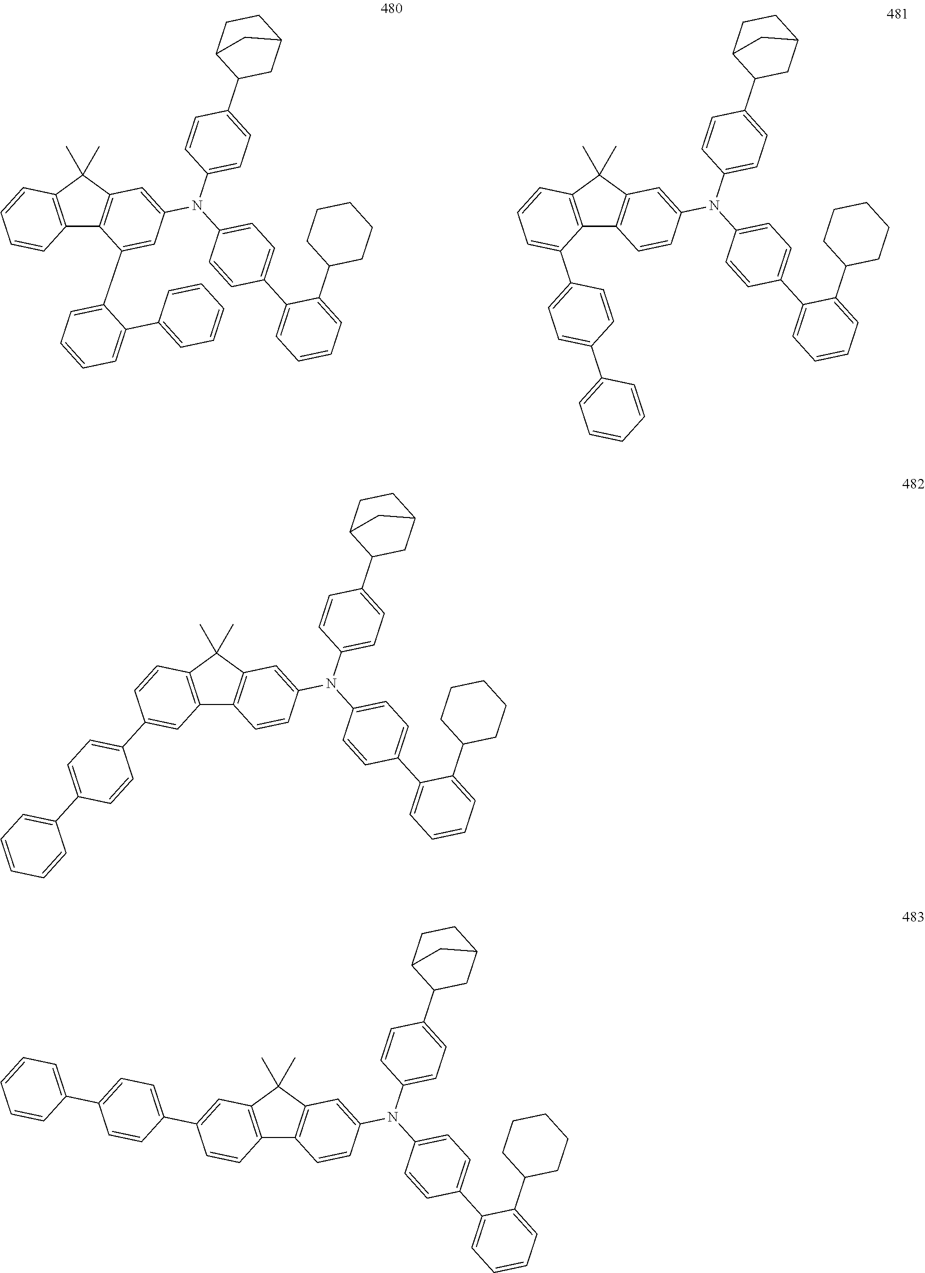 [SMILES: CC1(C)c2ccccc2-c2c(-c3ccccc3-c3ccccc3)cc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(-c4ccccc4C4CCCCC4)cc3)c3ccc(C4CC5CCC4C5)cc3)cc21 ; CC1(C)c2cc(-c3ccc(-c4ccccc4)cc3)ccc2-c2ccc(N(c3ccc(-c4ccccc4C4CCCCC4)cc3)c3ccc(C4CC5CCC4C5)cc3)cc21]

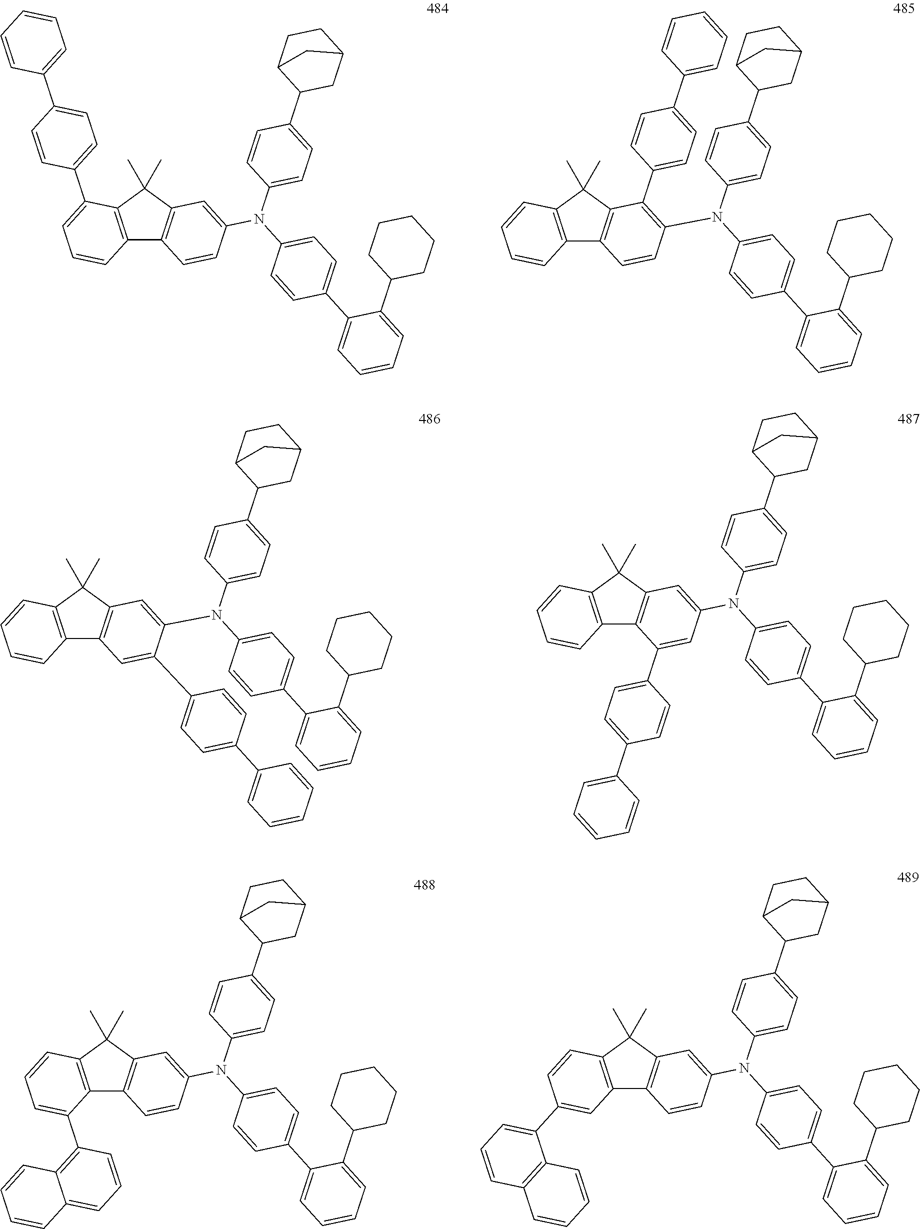

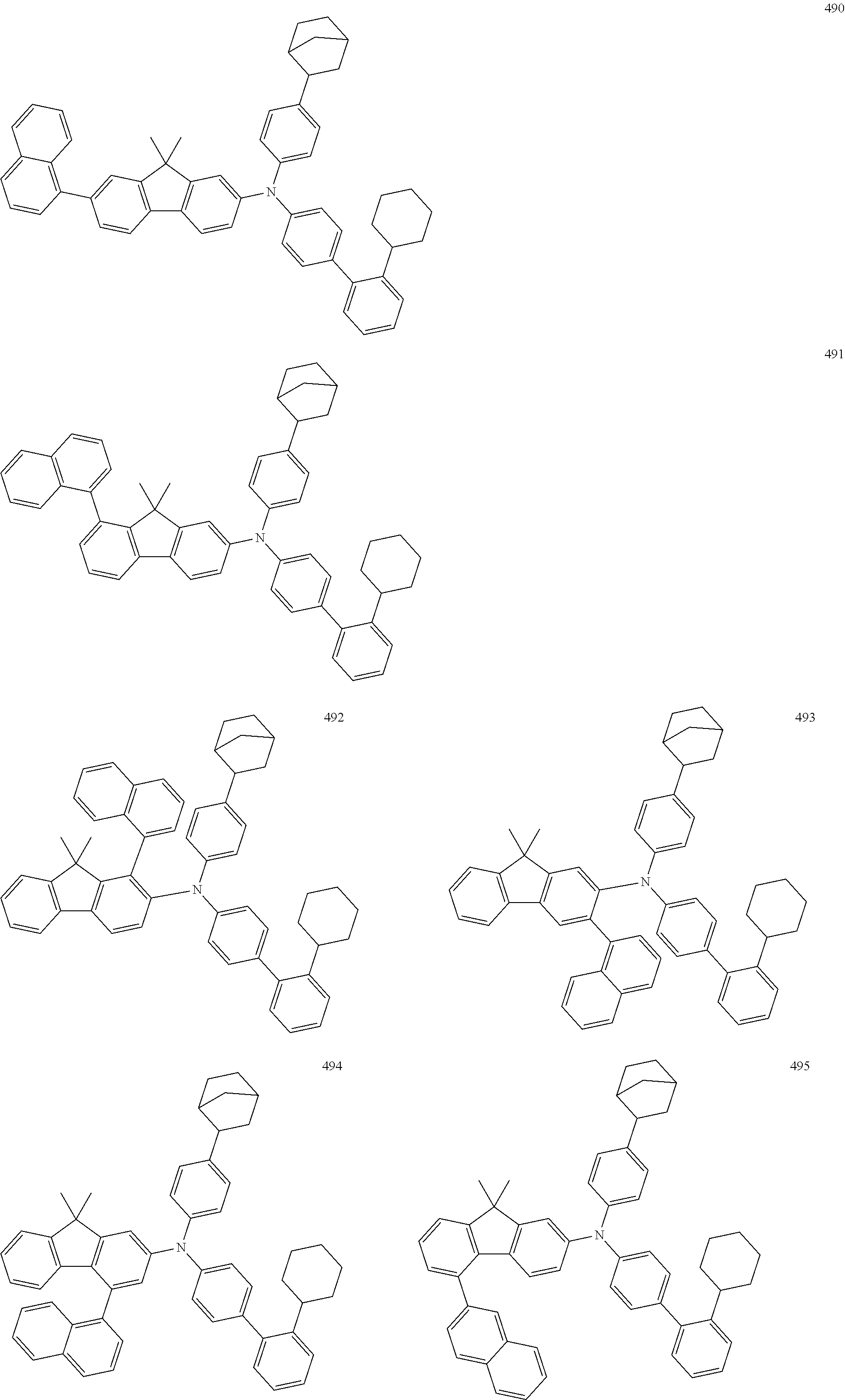

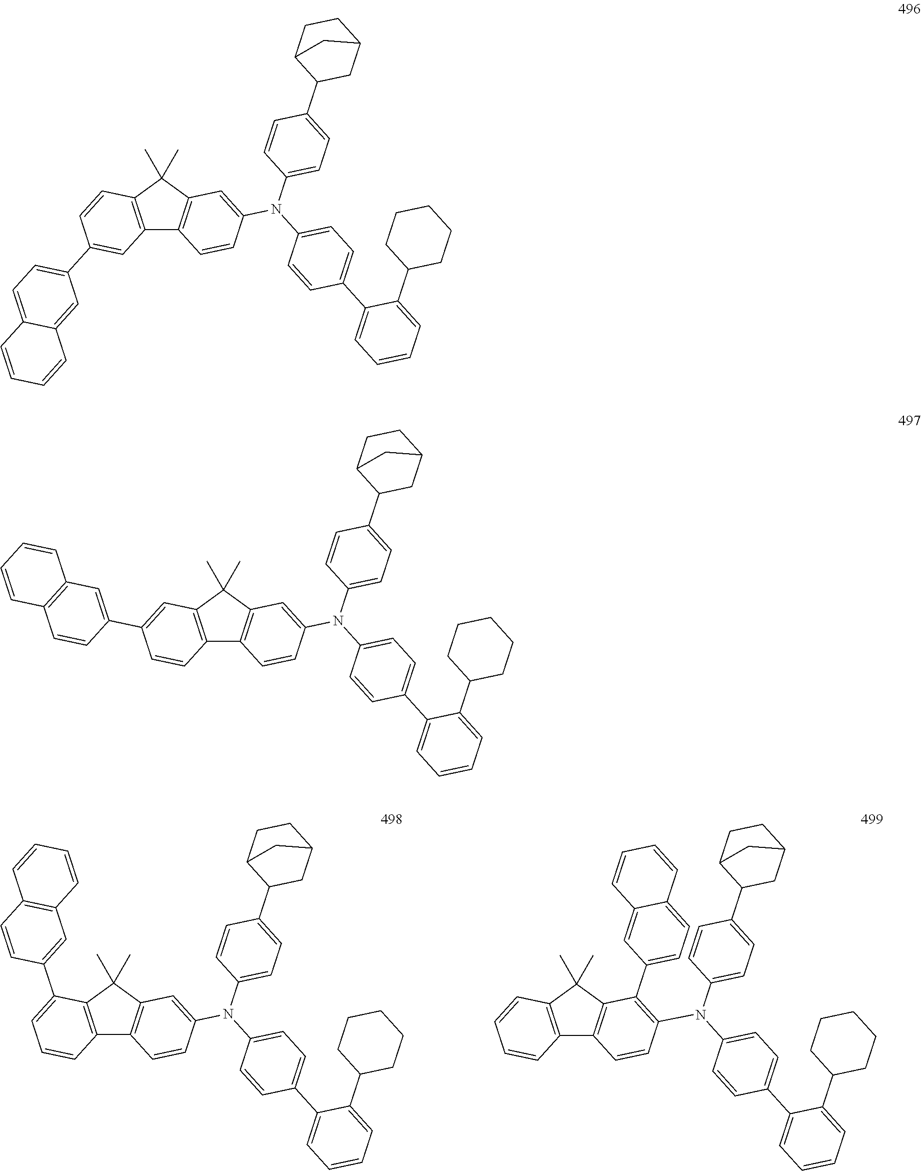 [SMILES: CC1(C)c2ccc(-c3ccc4ccccc4c3)cc2-c2ccc(N(c3ccc(-c4ccccc4C4CCCCC4)cc3)c3ccc(C4CC5CCC4C5)cc3)cc21 ; CC1(C)c2cc(-c3ccc4ccccc4c3)ccc2-c2ccc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2cccc(-c3ccc4ccccc4c3)c21 ; CC1(C)c2ccccc2-c2ccc(N(c3ccc(-c4ccccc4C4CCCCC4)cc3)c3ccc(C4CC5CCC4C5)cc3)c(-c3ccc4ccccc4c3)c21]

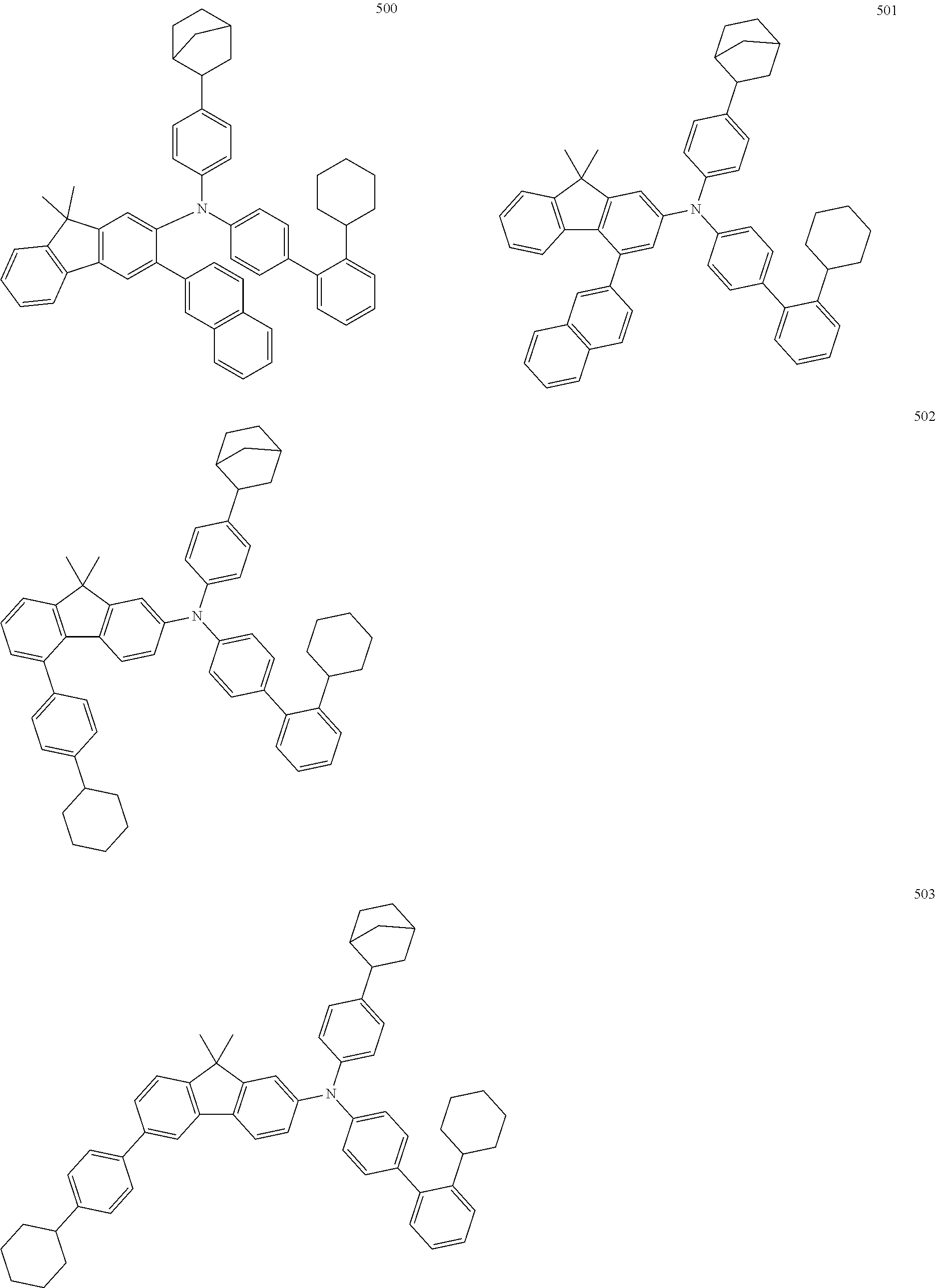 [SMILES: CC1(C)c2ccccc2-c2cc(-c3ccc4ccccc4c3)c(N(c3ccc(-c4ccccc4C4CCCCC4)cc3)c3ccc(C4CC5CCC4C5)cc3)cc21 ; CC1(C)c2ccccc2-c2c(-c3ccc4ccccc4c3)cc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(-c4ccccc4C4CCCCC4)cc3)c3ccc(C4CC5CCC4C5)cc3)cc21]

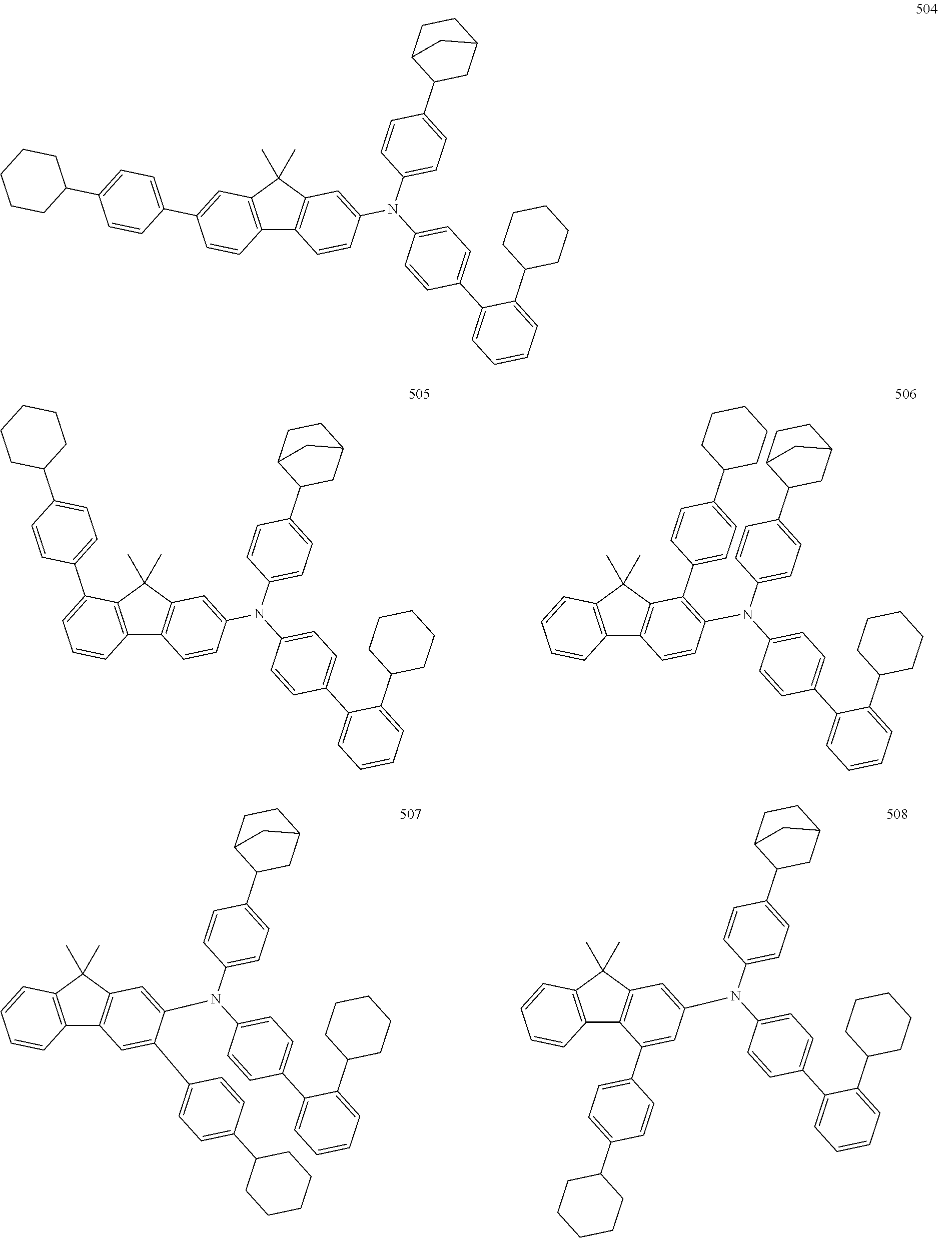

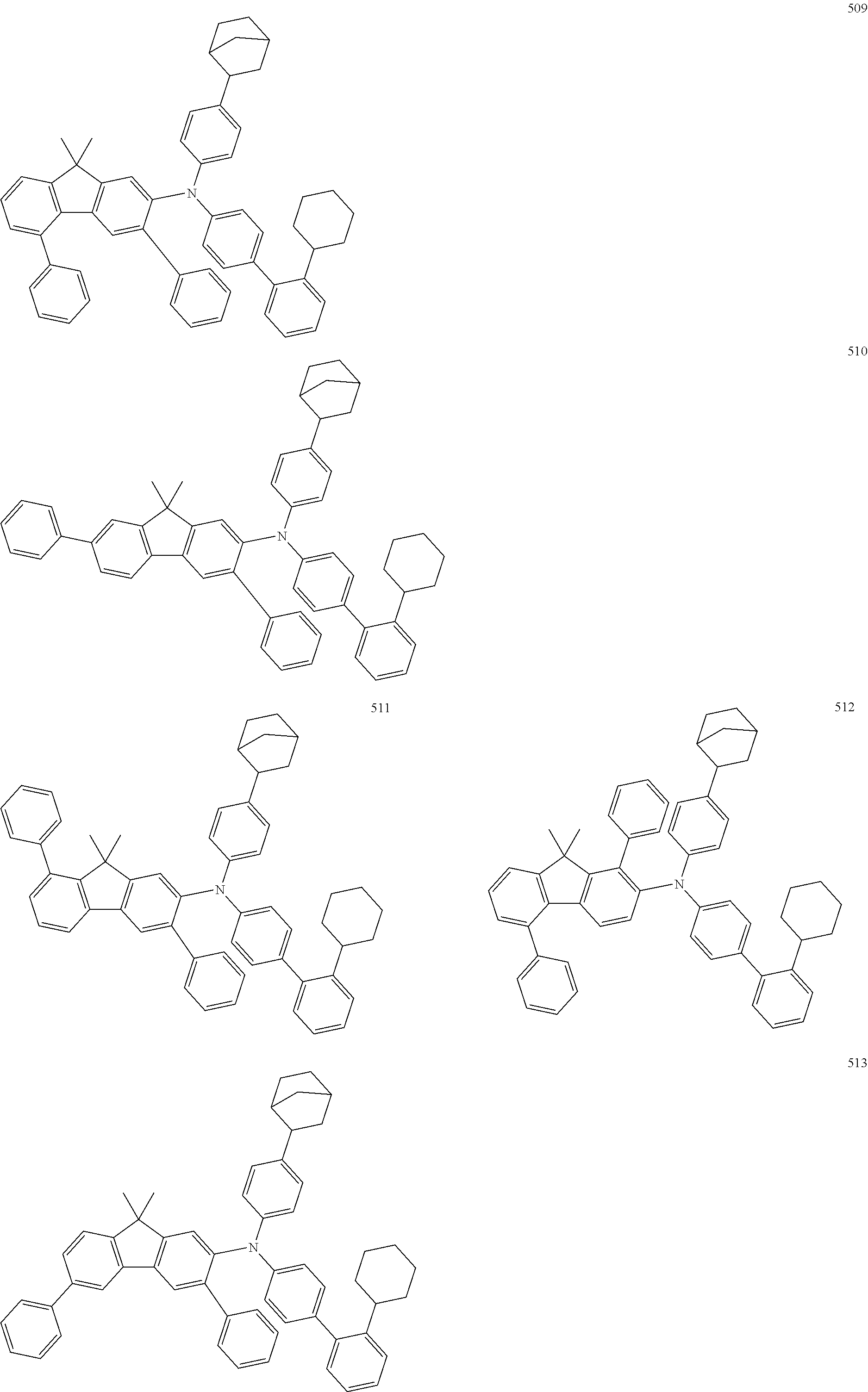

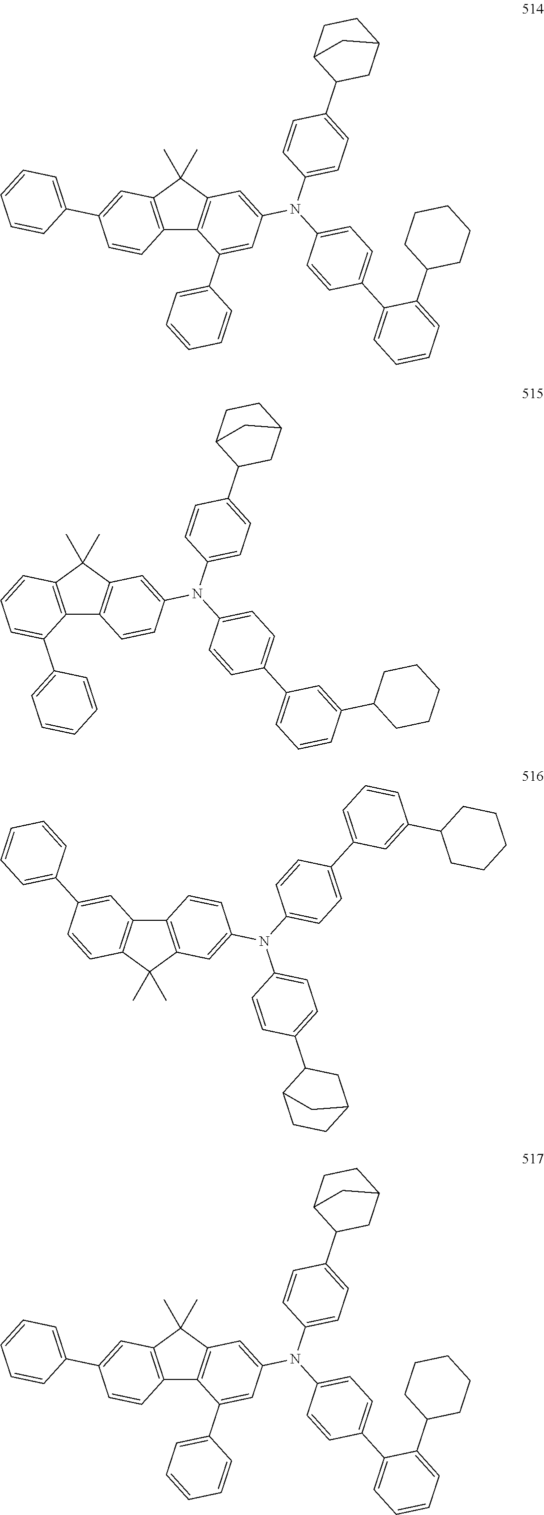

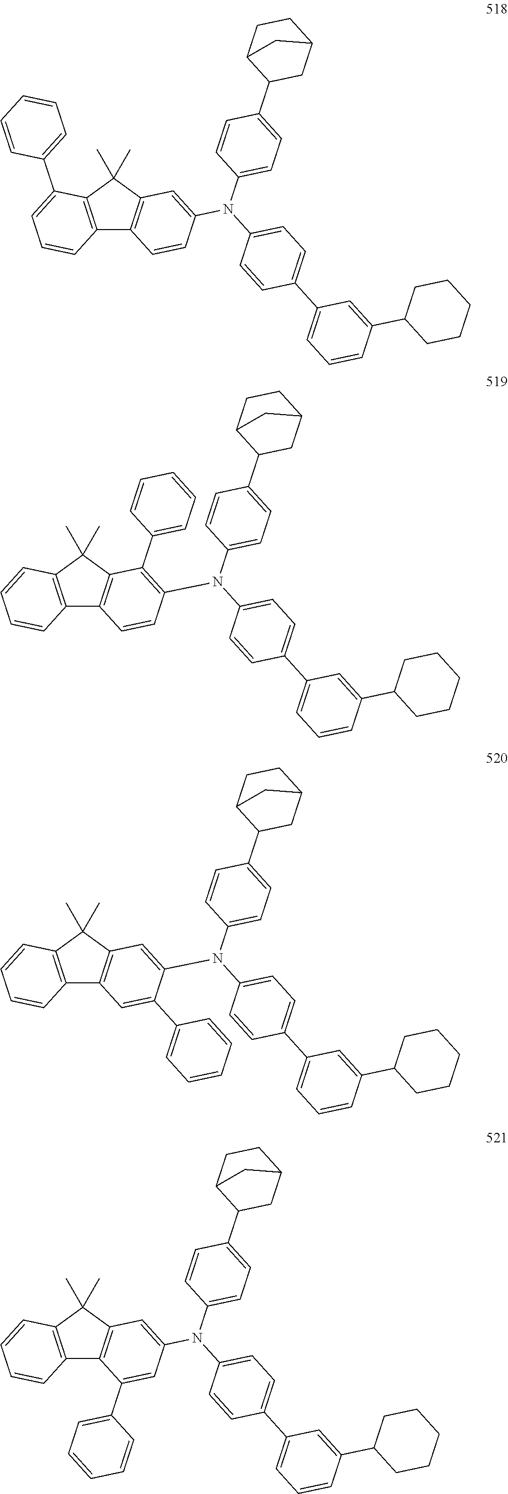 [SMILES: CC1(C)c2cc(N(c3ccc(-c4cccc(C5CCCCC5)c4)cc3)c3ccc(C4CC5CCC4C5)cc3)ccc2-c2cccc(-c3ccccc3)c21 ; CC1(C)c2ccccc2-c2ccc(N(c3ccc(-c4cccc(C5CCCCC5)c4)cc3)c3ccc(C4CC5CCC4C5)cc3)c(-c3ccccc3)c21 ; CC1(C)c2ccccc2-c2cc(-c3ccccc3)c(N(c3ccc(-c4cccc(C5CCCCC5)c4)cc3)c3ccc(C4CC5CCC4C5)cc3)cc21 ; CC1(C)c2ccccc2-c2c(-c3ccccc3)cc(N(c3ccc(-c4cccc(C5CCCCC5)c4)cc3)c3ccc(C4CC5CCC4C5)cc3)cc21]

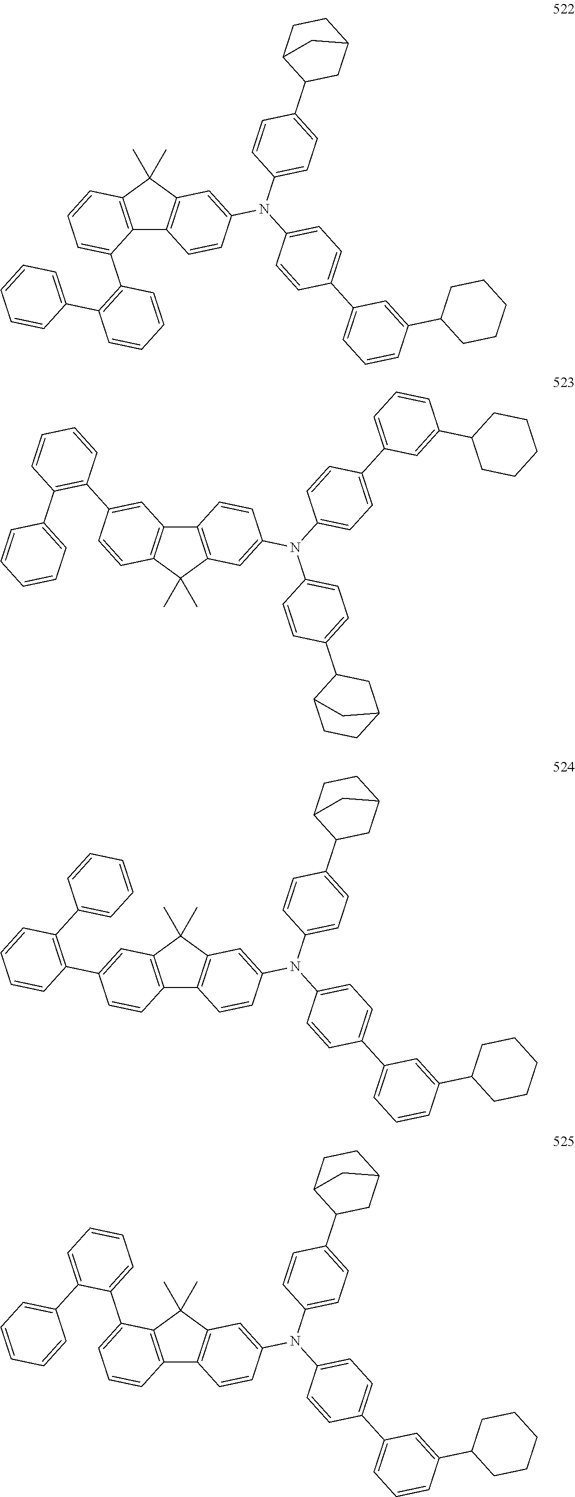

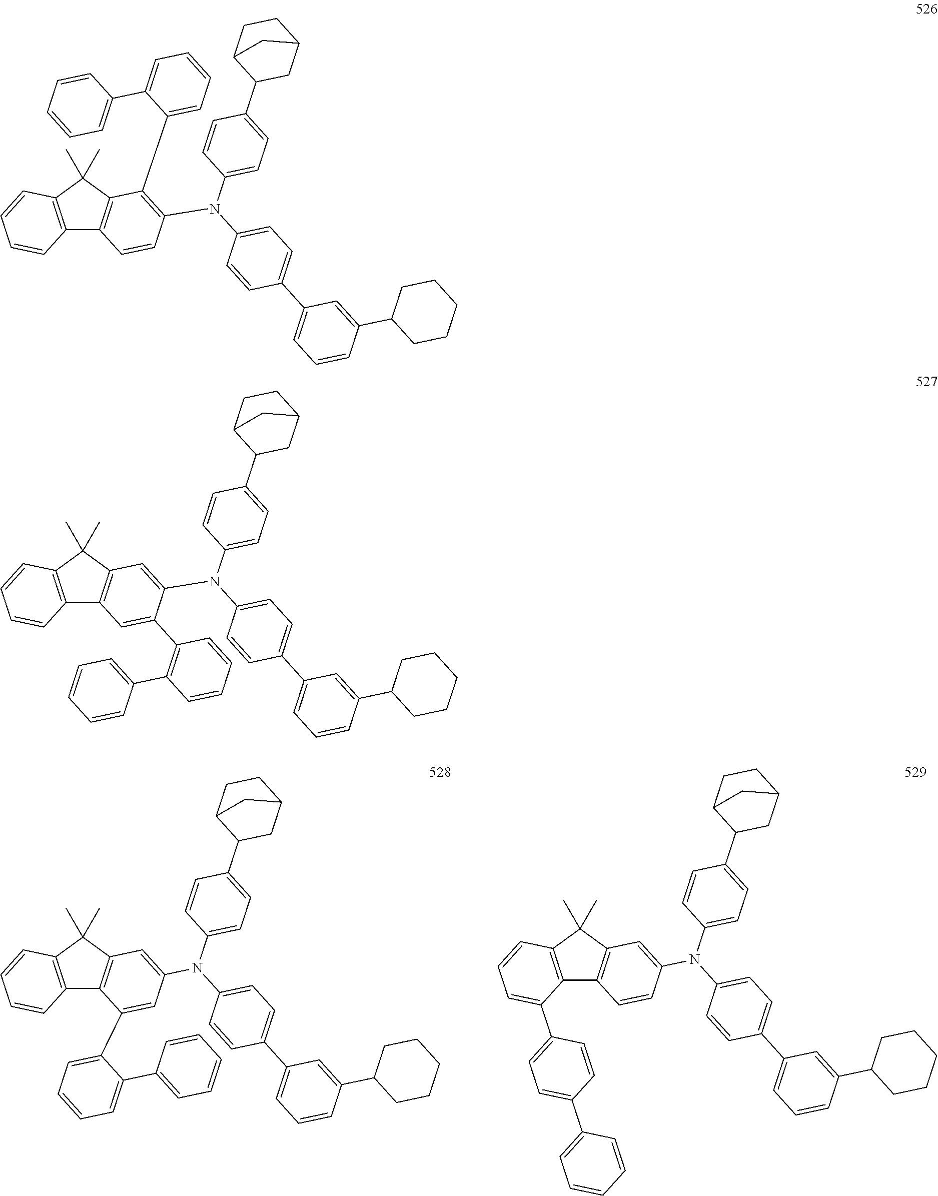 [SMILES: CC1(C)c2ccccc2-c2ccc(N(c3ccc(-c4cccc(C5CCCCC5)c4)cc3)c3ccc(C4CC5CCC4C5)cc3)c(-c3ccccc3-c3ccccc3)c21 ; CC1(C)c2ccccc2-c2cc(-c3ccccc3-c3ccccc3)c(N(c3ccc(-c4cccc(C5CCCCC5)c4)cc3)c3ccc(C4CC5CCC4C5)cc3)cc21 ; CC1(C)c2ccccc2-c2c(-c3ccccc3-c3ccccc3)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2c(-c3ccc(-c4ccccc4)cc3)cccc21]

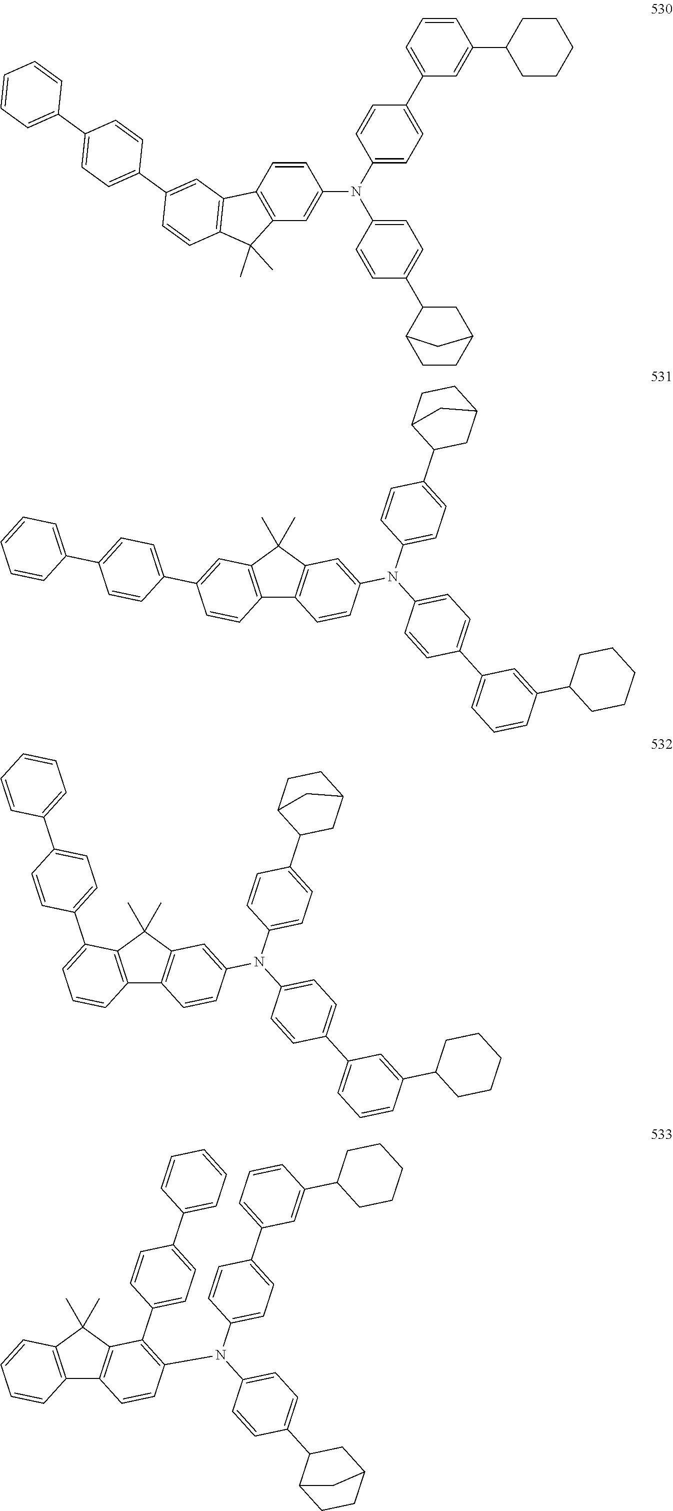

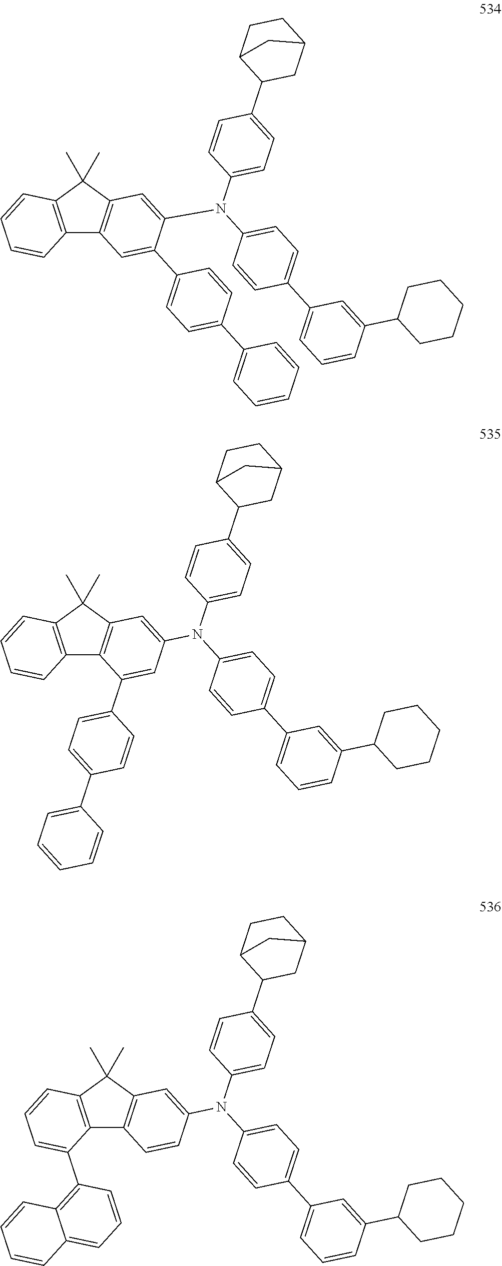

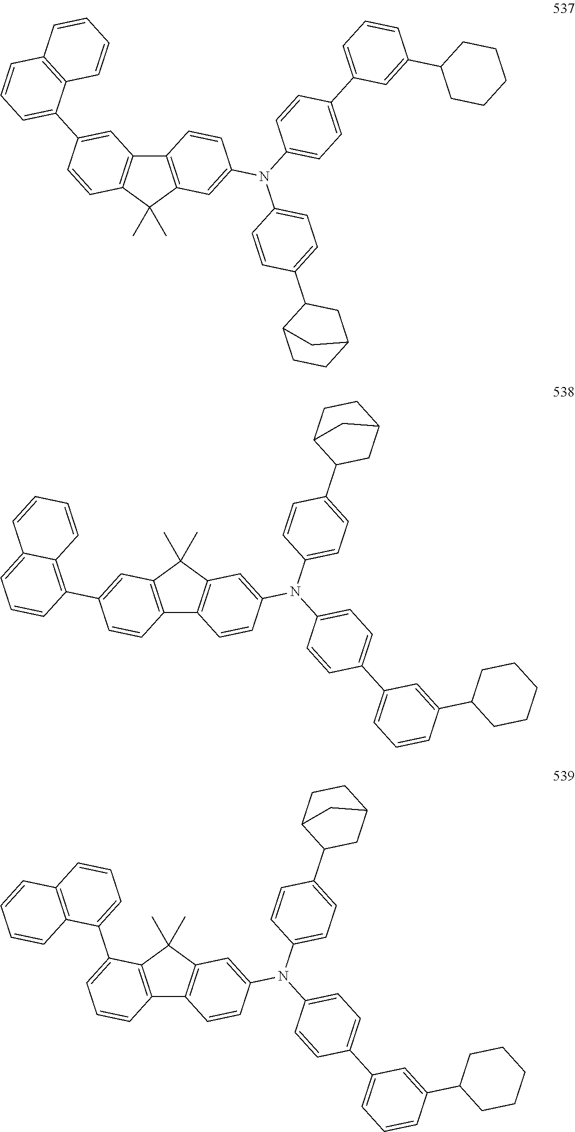

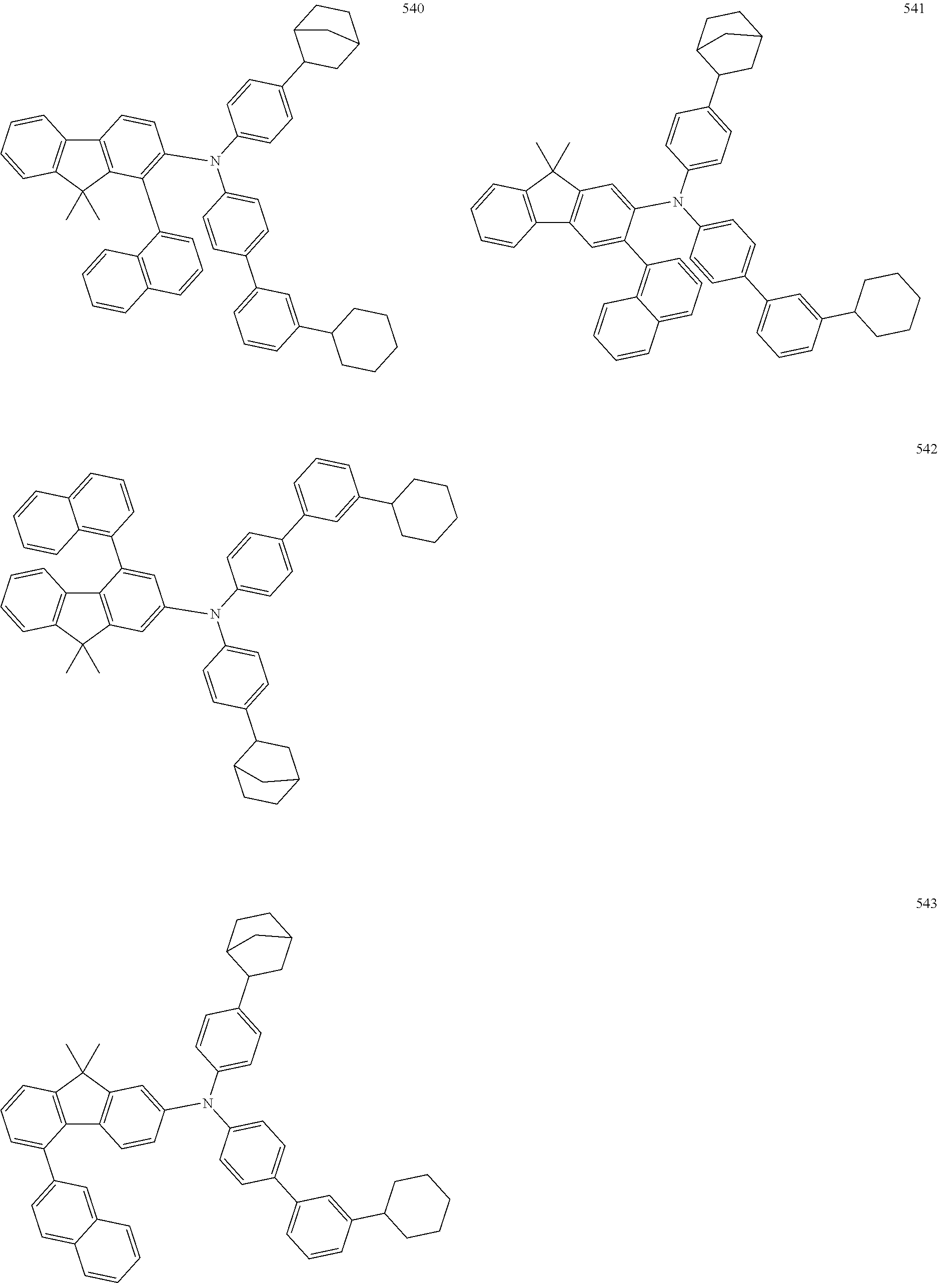 [SMILES: CC1(C)c2ccccc2-c2ccc(N(c3ccc(-c4cccc(C5CCCCC5)c4)cc3)c3ccc(C4CC5CCC4C5)cc3)c(-c3cccc4ccccc34)c21 ; CC1(C)c2ccccc2-c2cc(-c3cccc4ccccc34)c(N(c3ccc(-c4cccc(C5CCCCC5)c4)cc3)c3ccc(C4CC5CCC4C5)cc3)cc21 ; CC1(C)c2ccccc2-c2c(-c3cccc4ccccc34)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2c(-c3ccc4ccccc4c3)cccc21]

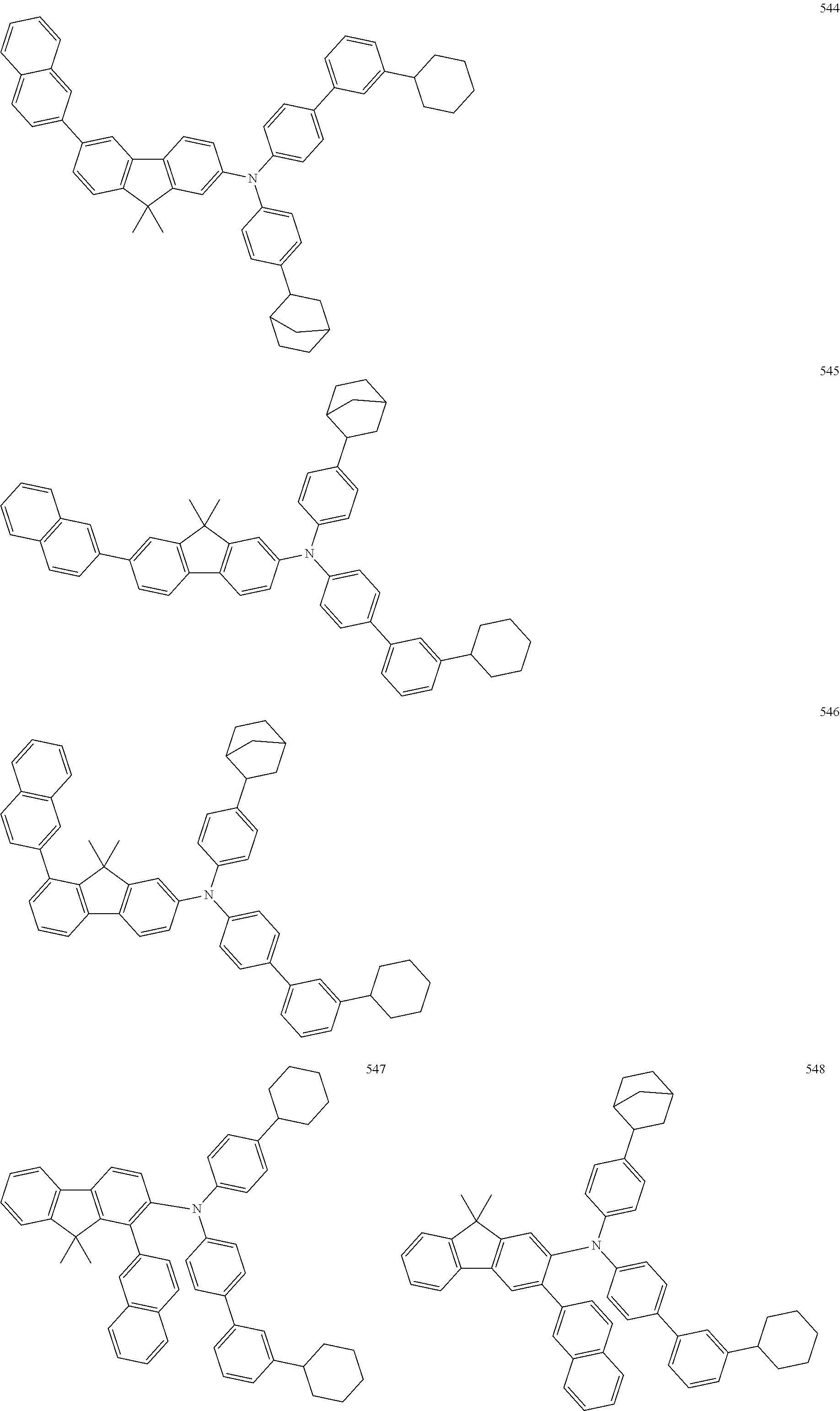

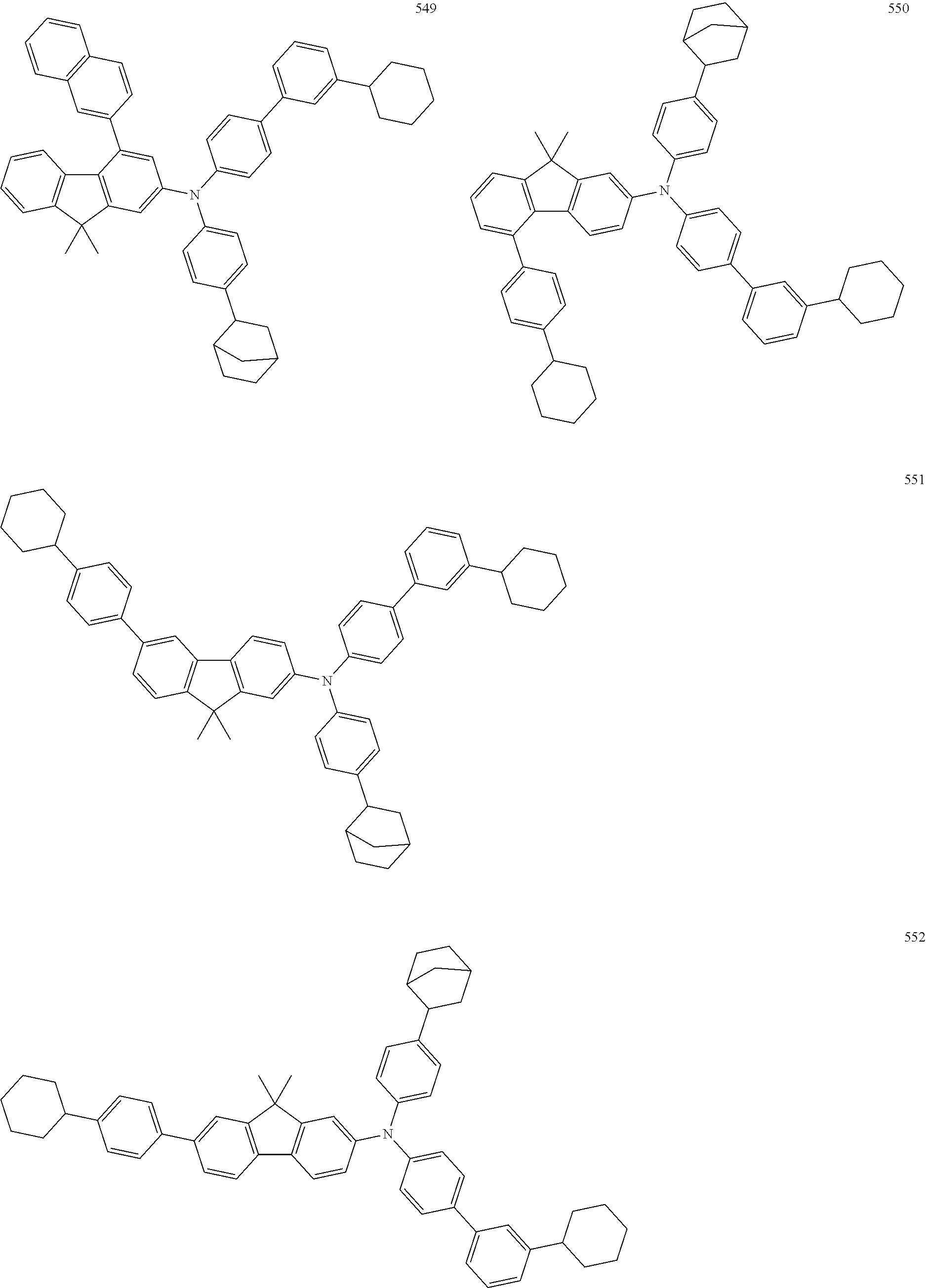 [SMILES: CC1(C)c2ccccc2-c2c(-c3ccc4ccccc4c3)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(-c4cccc(C5CCCCC5)c4)cc3)c3ccc(C4CC5CCC4C5)cc3)cc21 ; CC1(C)c2cc(-c3ccc(C4CCCCC4)cc3)ccc2-c2ccc(N(c3ccc(-c4cccc(C5CCCCC5)c4)cc3)c3ccc(C4CC5CCC4C5)cc3)cc21]

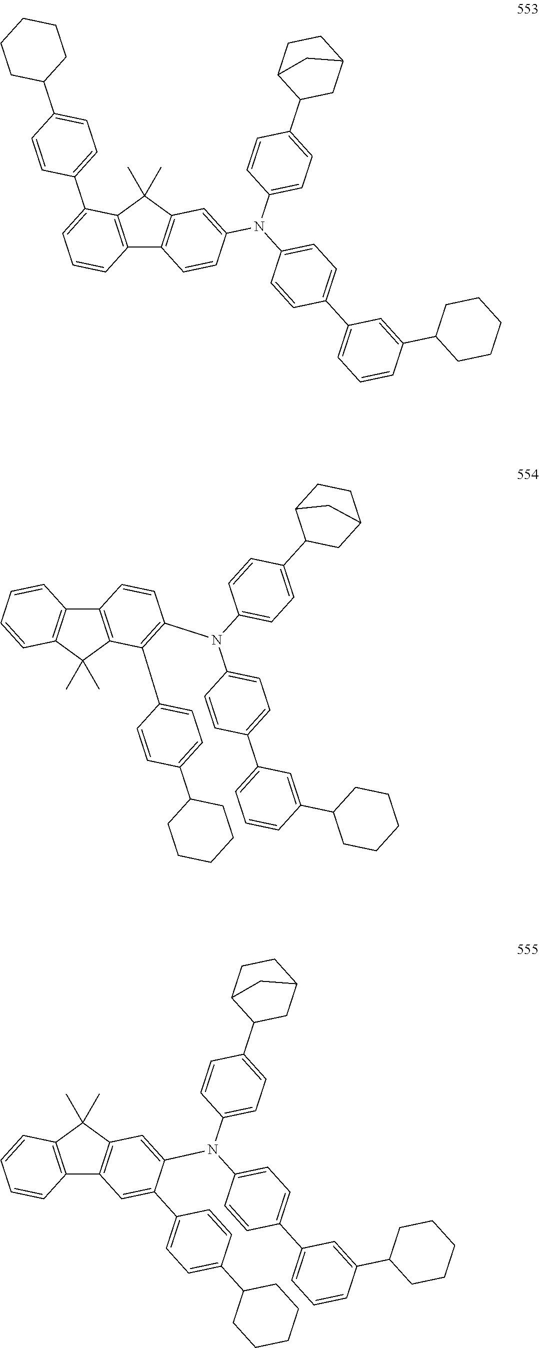 [SMILES: CC1(C)c2cc(N(c3ccc(-c4cccc(C5CCCCC5)c4)cc3)c3ccc(C4CC5CCC4C5)cc3)ccc2-c2cccc(-c3ccc(C4CCCCC4)cc3)c21 ; CC1(C)c2ccccc2-c2ccc(N(c3ccc(-c4cccc(C5CCCCC5)c4)cc3)c3ccc(C4CC5CCC4C5)cc3)c(-c3ccc(C4CCCCC4)cc3)c21 ; CC1(C)c2ccccc2-c2cc(-c3ccc(C4CCCCC4)cc3)c(N(c3ccc(-c4cccc(C5CCCCC5)c4)cc3)c3ccc(C4CC5CCC4C5)cc3)cc21]

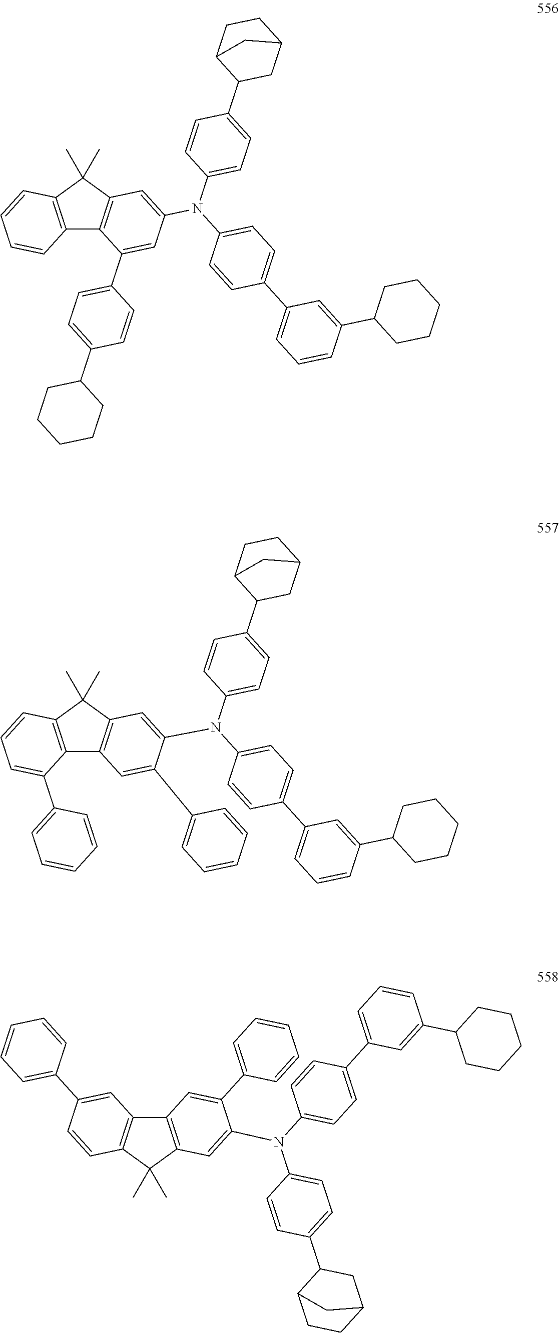 [SMILES: CC1(C)c2ccccc2-c2c(-c3ccc(C4CCCCC4)cc3)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(-c4cccc(C5CCCCC5)c4)cc3)c3ccc(C4CC5CCC4C5)cc3)cc21]

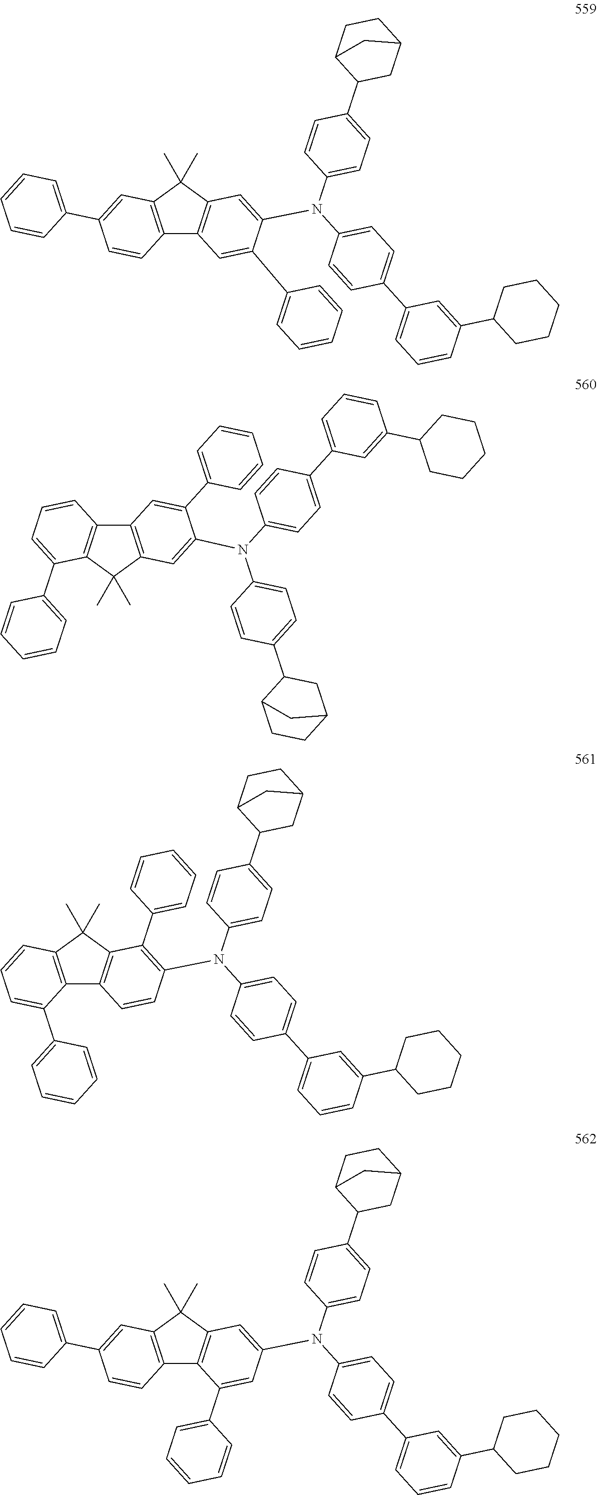

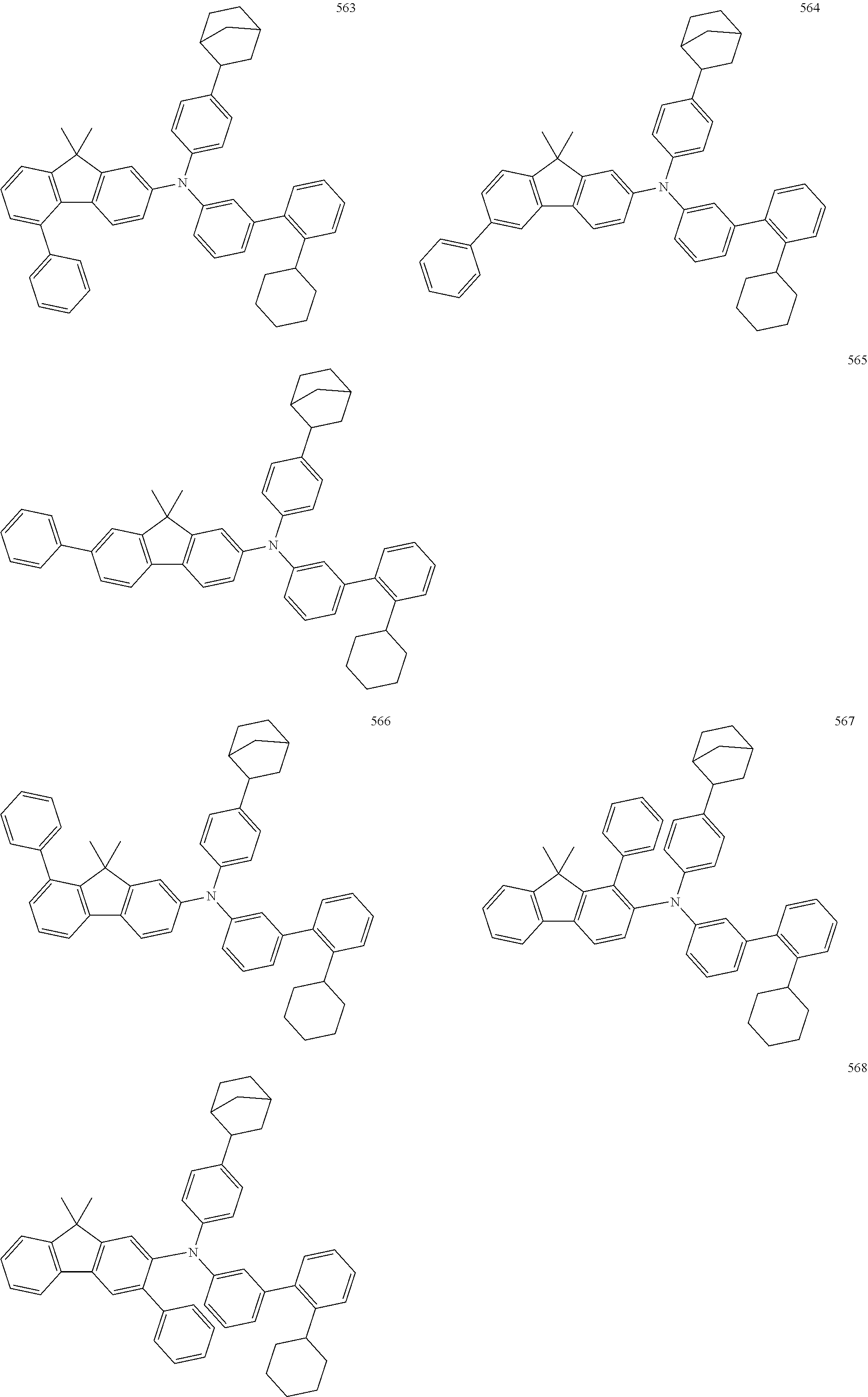

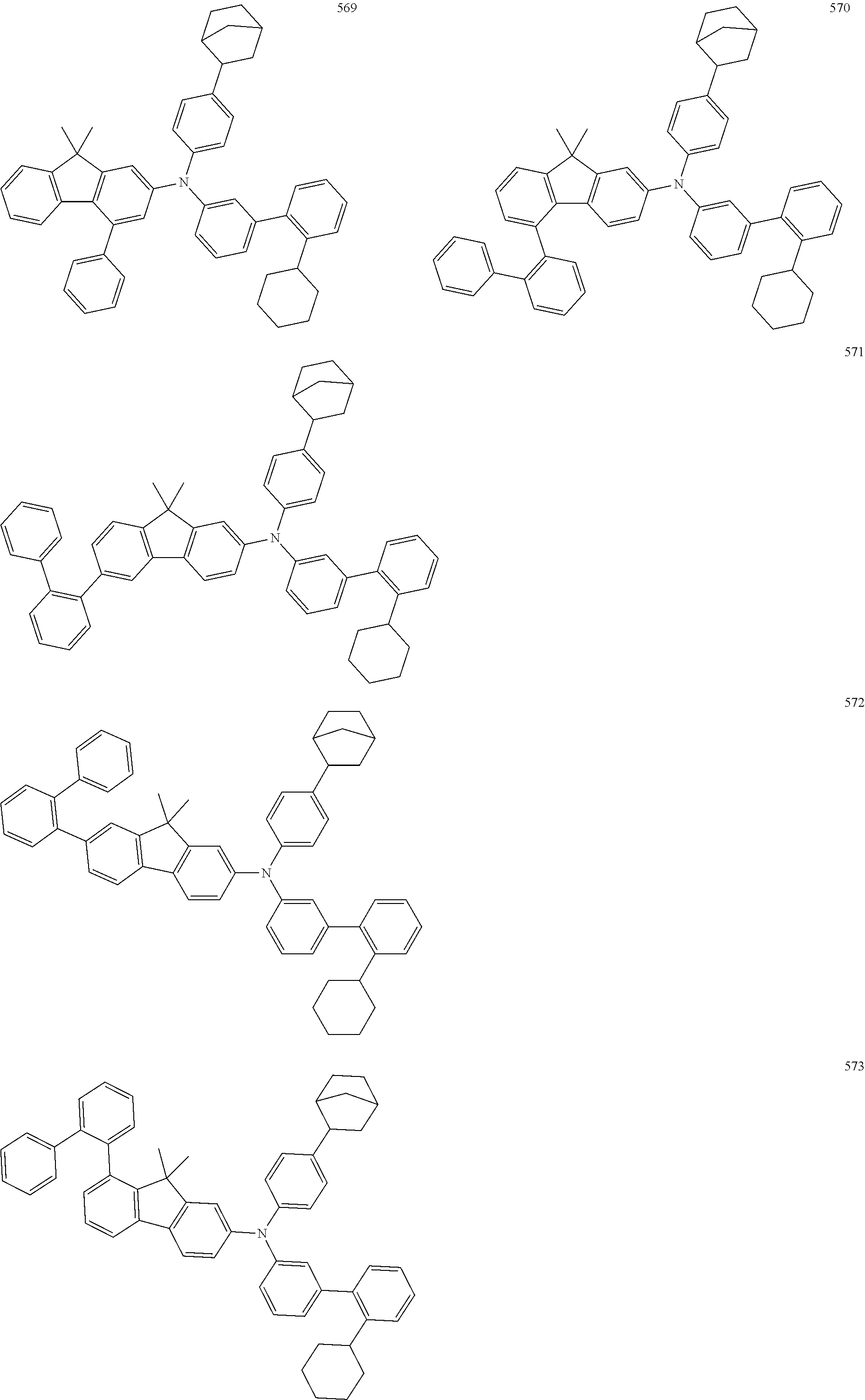

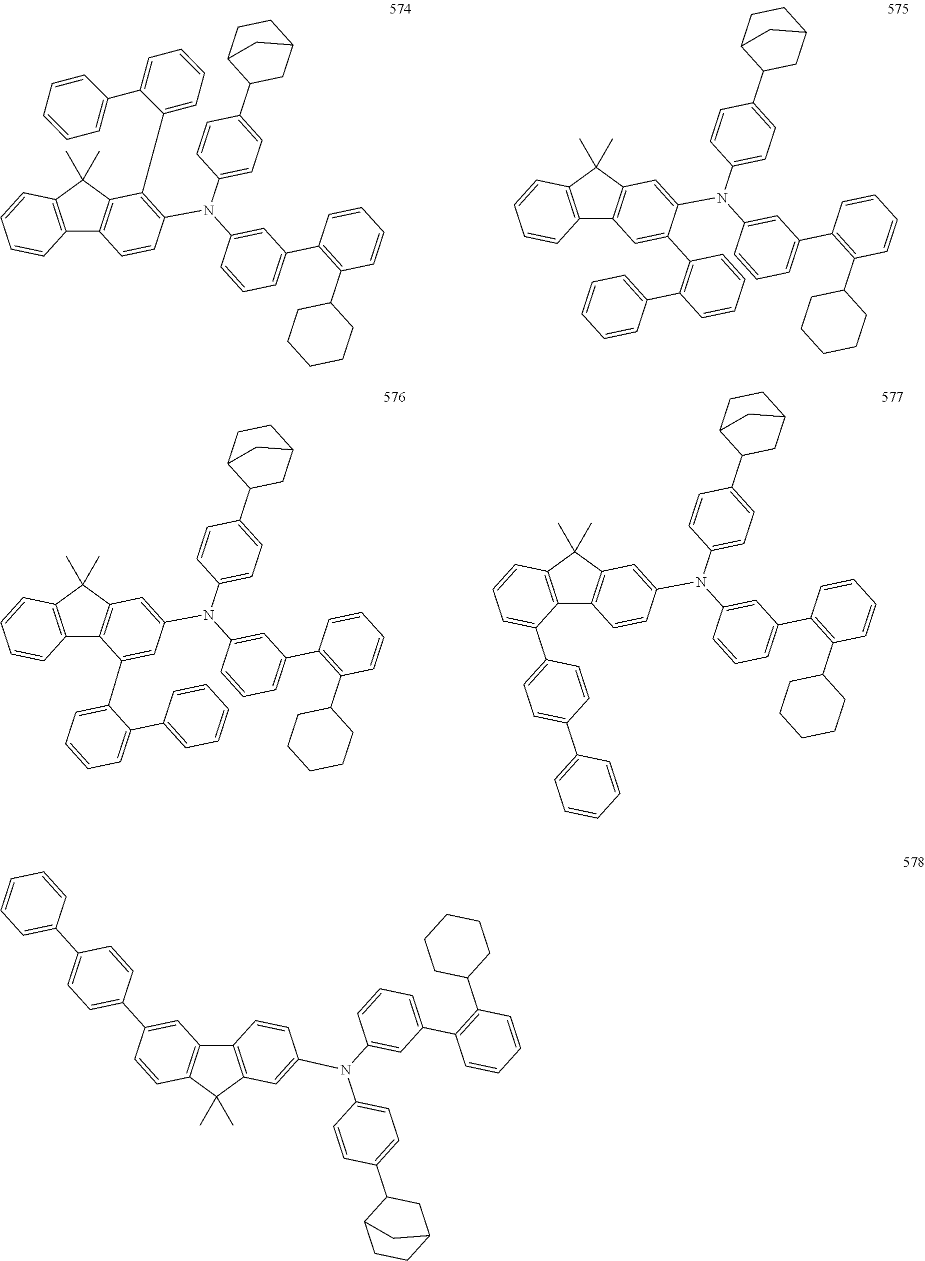

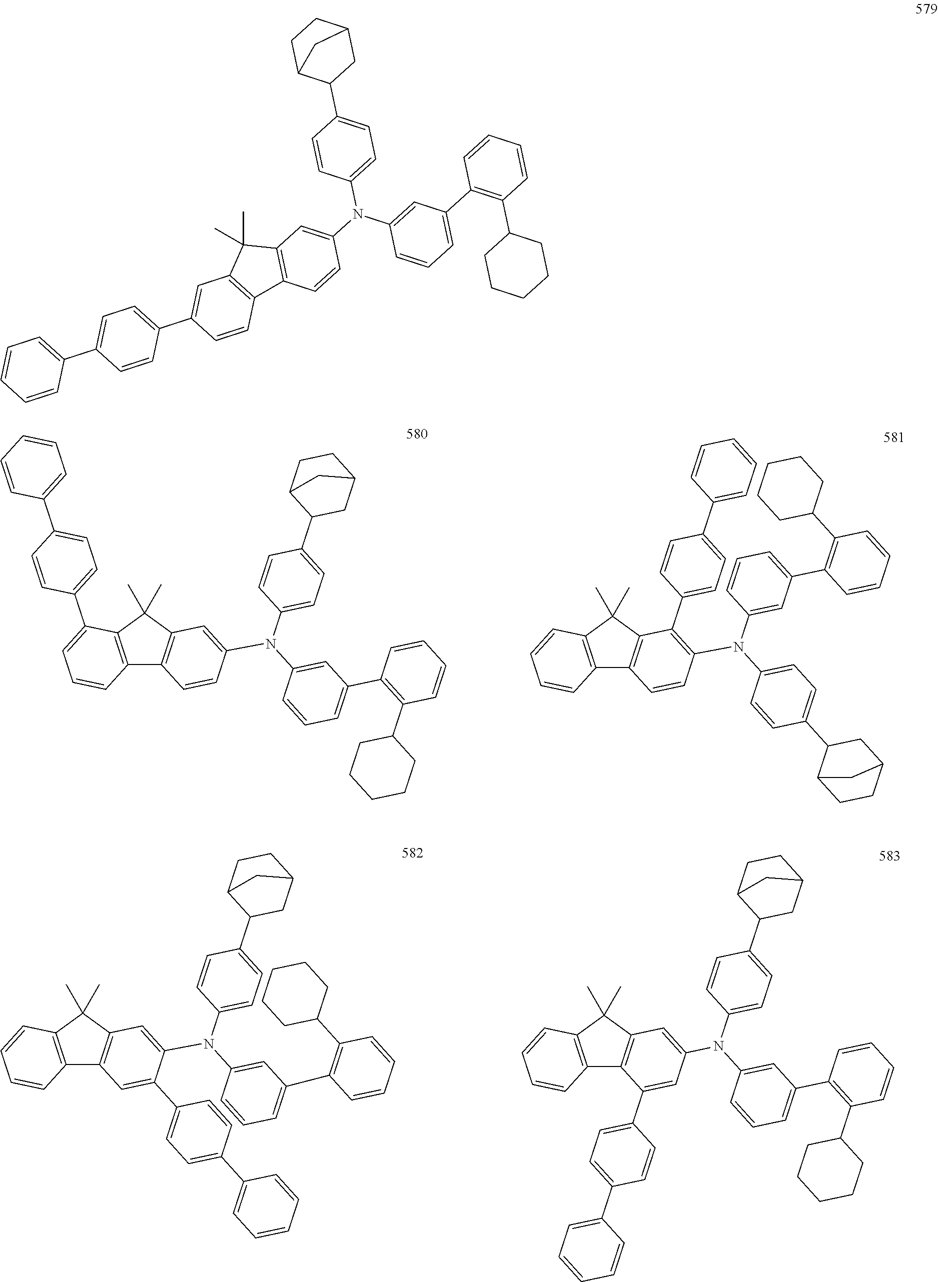

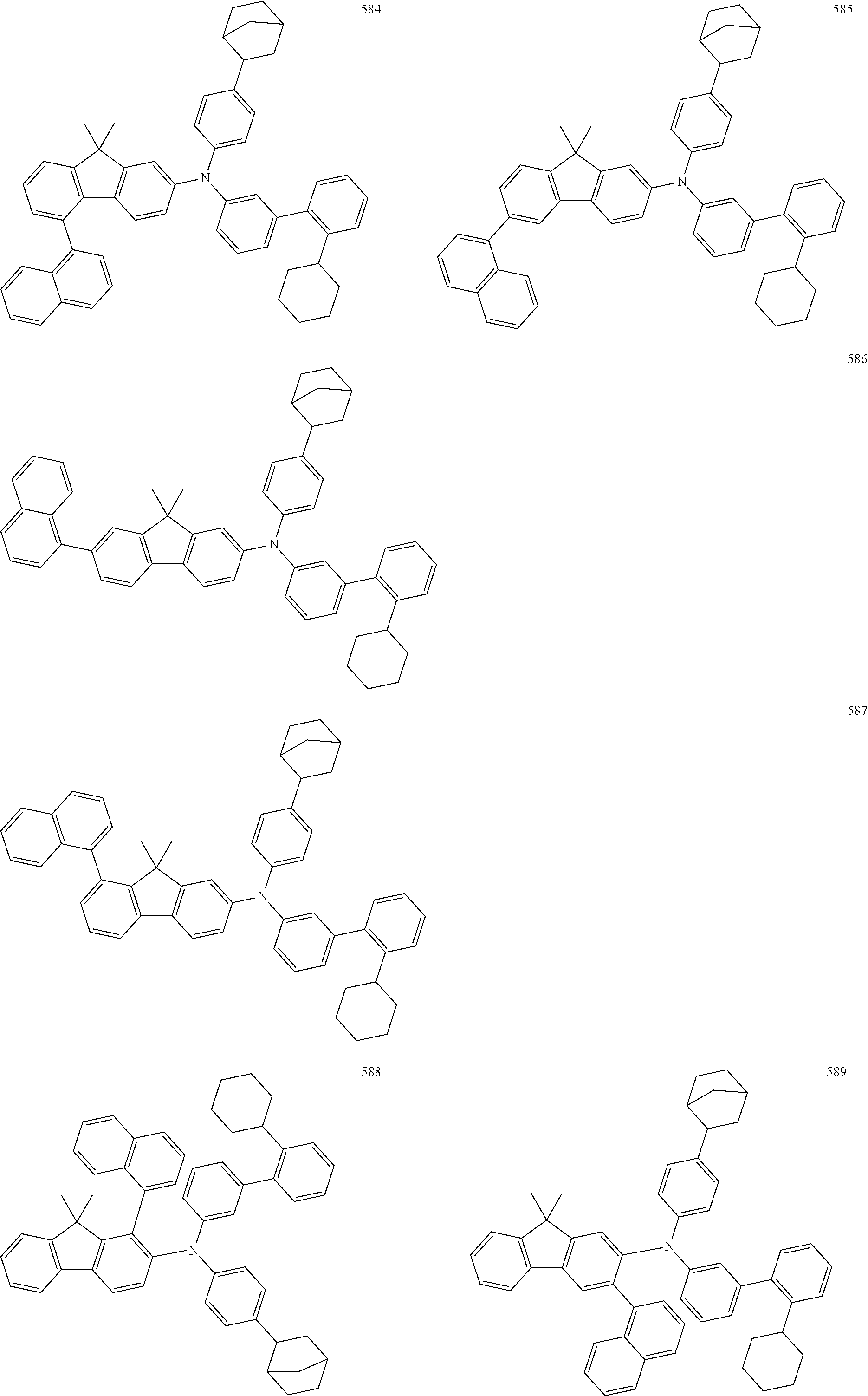

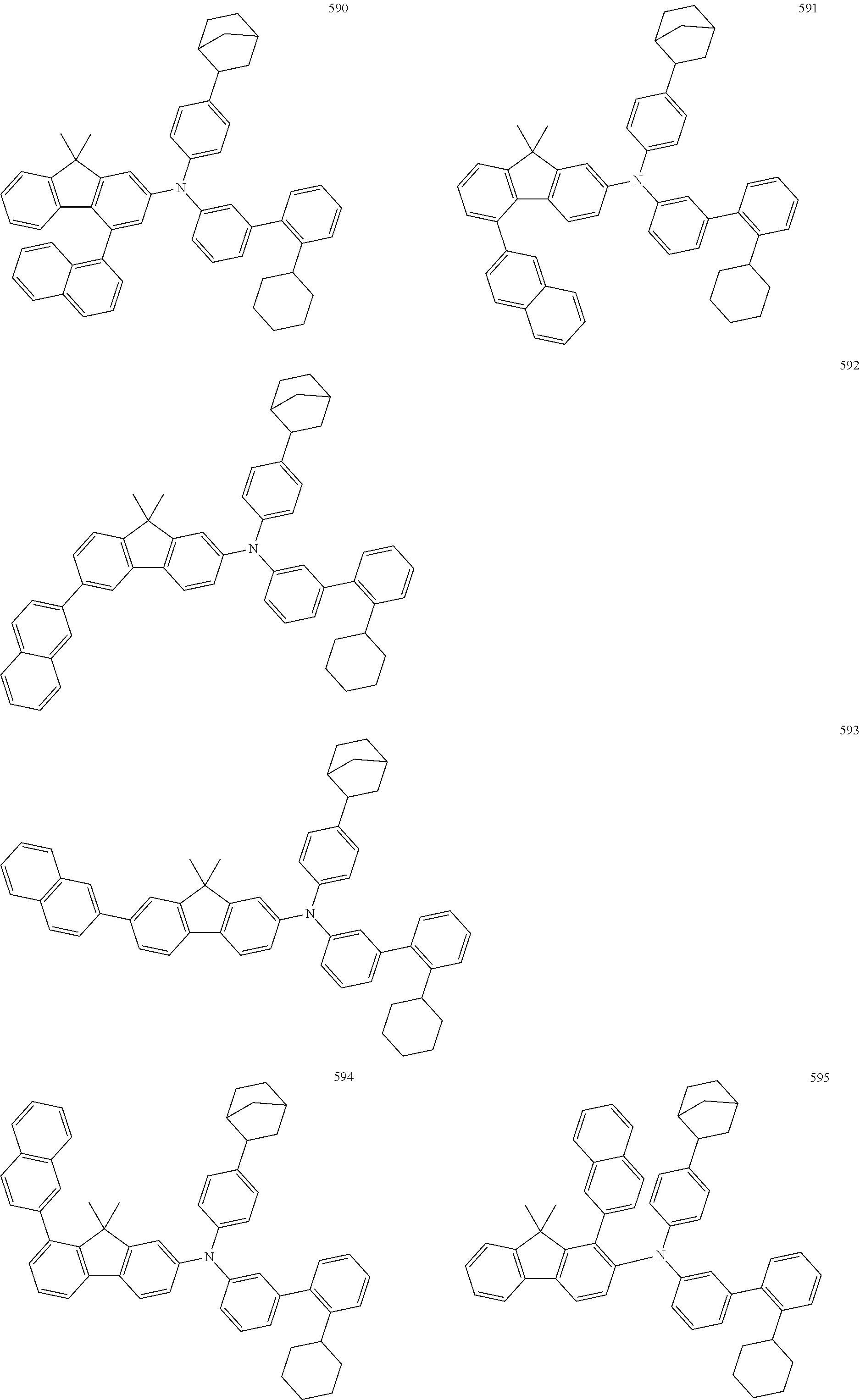

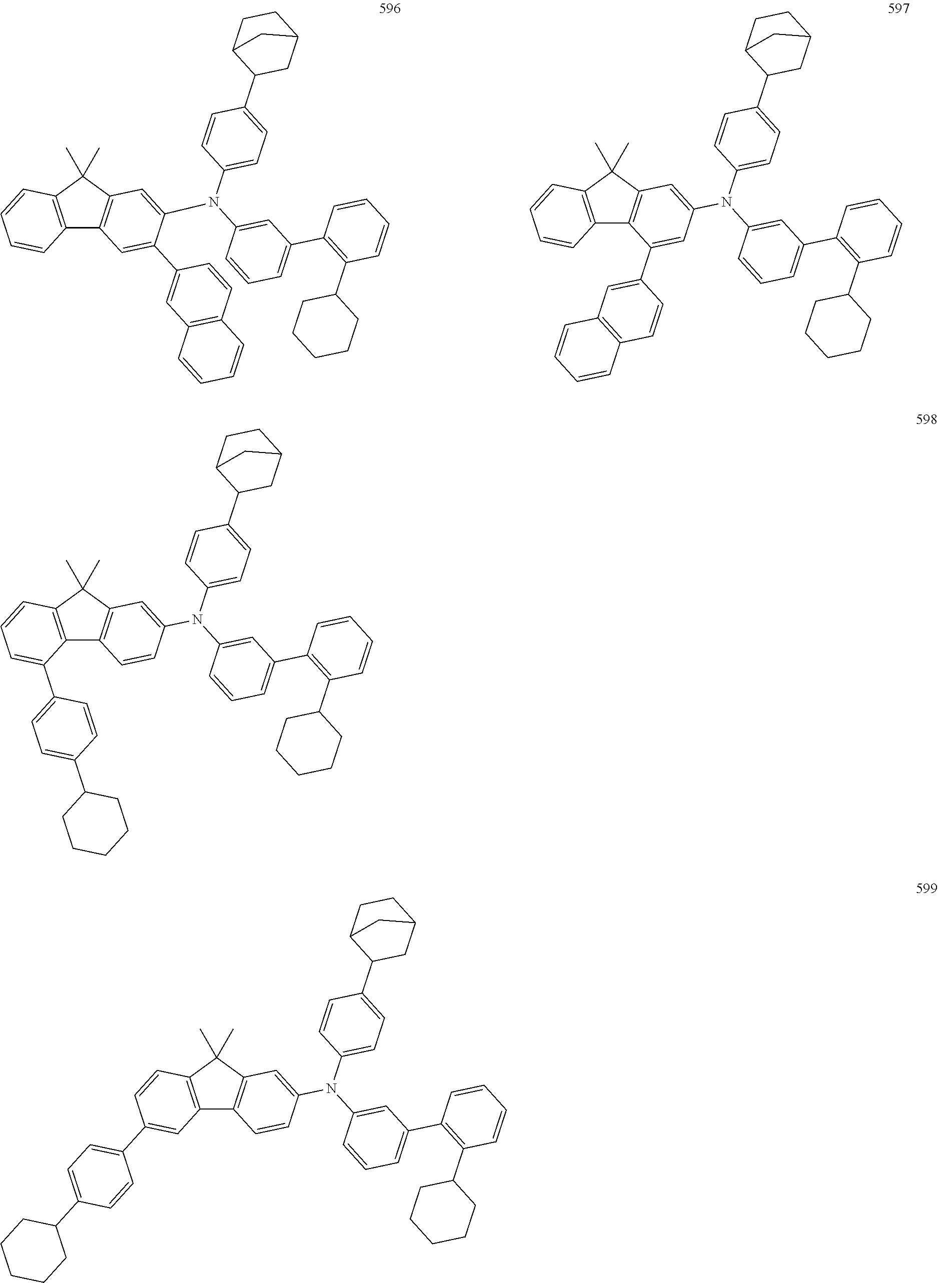 [SMILES: CC1(C)c2ccccc2-c2cc(-c3ccc4ccccc4c3)c(N(c3ccc(C4CC5CCC4C5)cc3)c3cccc(-c4ccccc4C4CCCCC4)c3)cc21 ; CC1(C)c2ccccc2-c2c(-c3ccc4ccccc4c3)cc(N(c3ccc(C4CC5CCC4C5)cc3)c3cccc(-c4ccccc4C4CCCCC4)c3)cc21 ; CC1(C)c2cc(N(c3ccc(C4CC5CCC4C5)cc3)c3cccc(-c4ccccc4C4CCCCC4)c3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cccc(-c4ccccc4C4CCCCC4)c3)cc21]

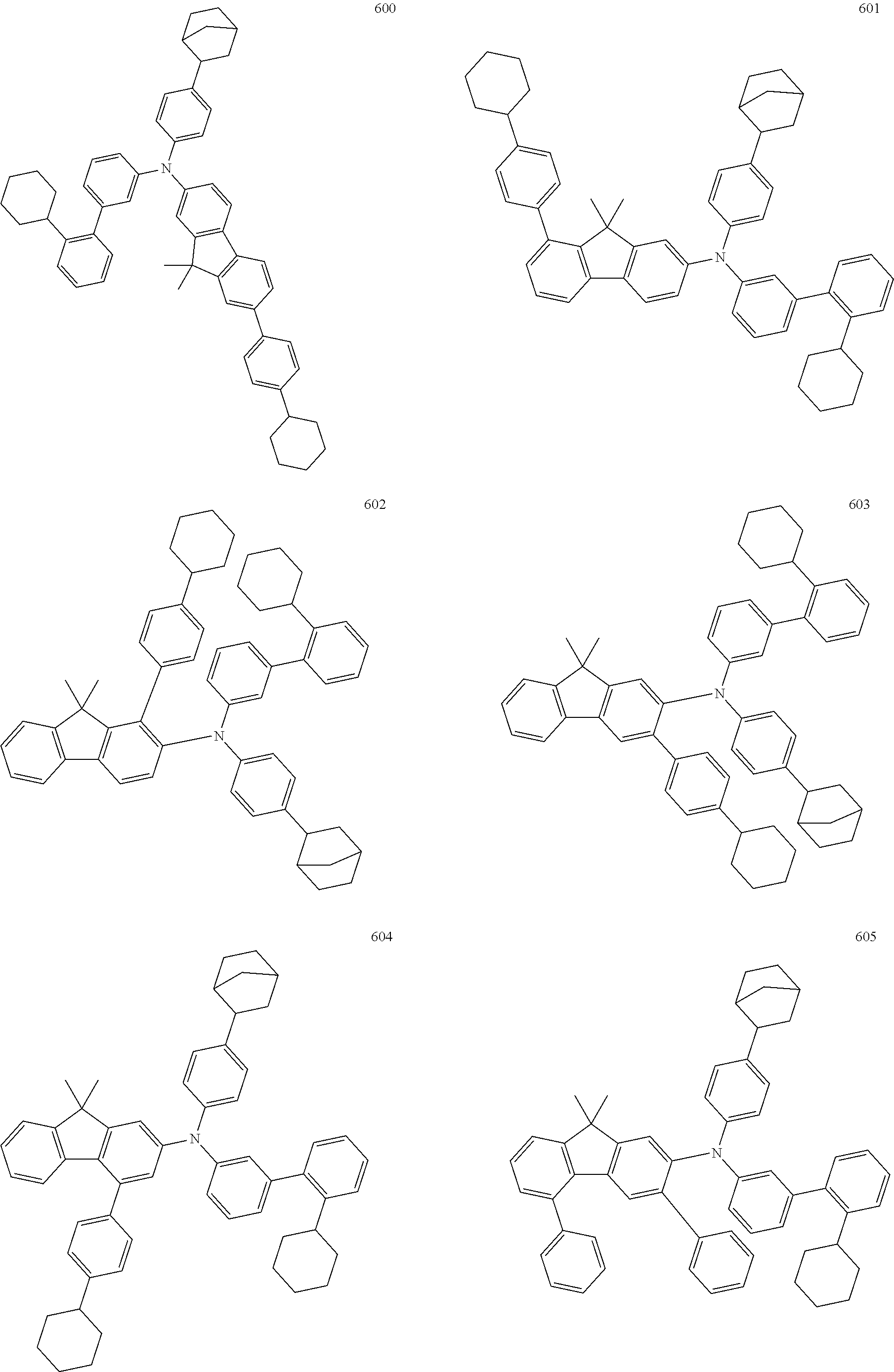

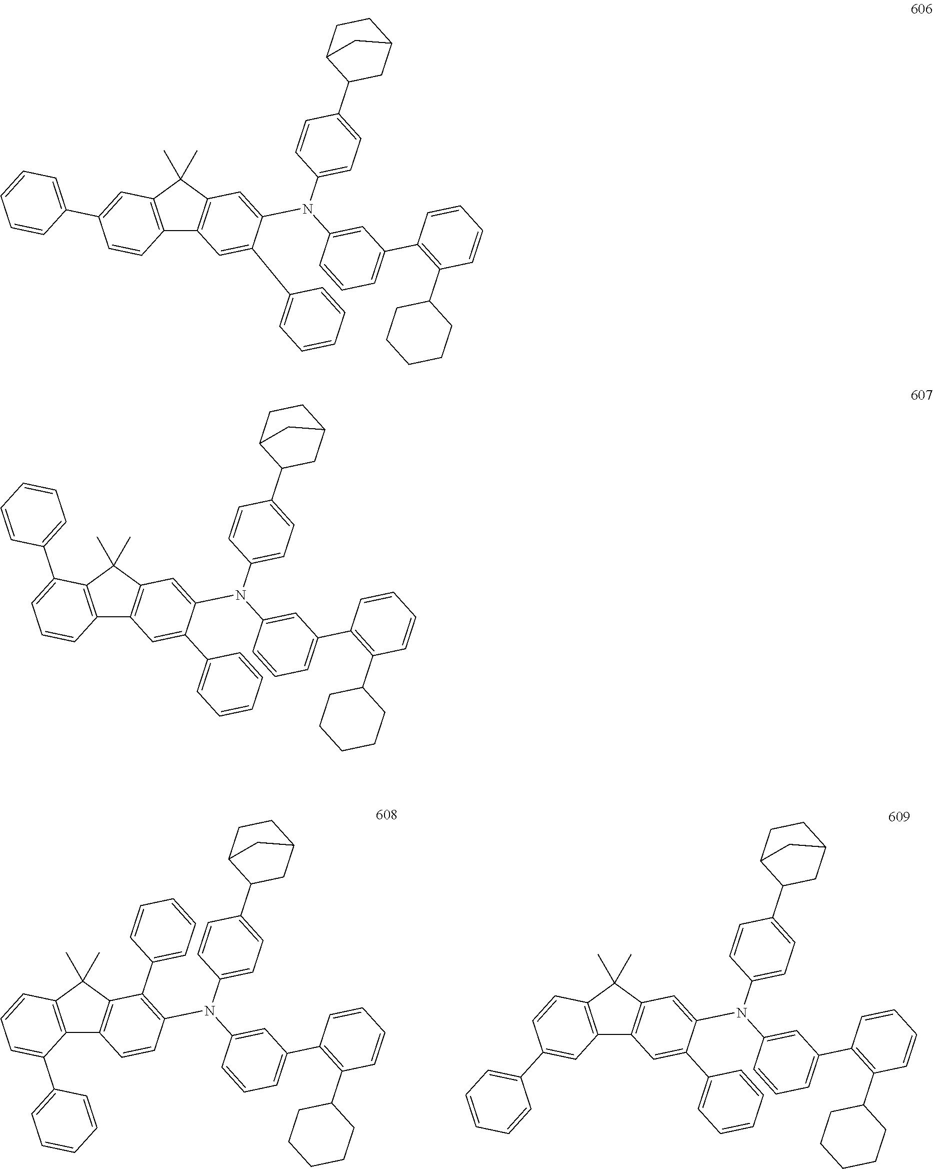 [SMILES: CC1(C)c2cc(-c3ccccc3)ccc2-c2cc(-c3ccccc3)c(N(c3ccc(C4CC5CCC4C5)cc3)c3cccc(-c4ccccc4C4CCCCC4)c3)cc21 ; CC1(C)c2cc(N(c3ccc(C4CC5CCC4C5)cc3)c3cccc(-c4ccccc4C4CCCCC4)c3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3cccc(-c4ccccc4C4CCCCC4)c3)c(-c3ccccc3)c21 ; CC1(C)c2ccc(-c3ccccc3)cc2-c2cc(-c3ccccc3)c(N(c3ccc(C4CC5CCC4C5)cc3)c3cccc(-c4ccccc4C4CCCCC4)c3)cc21]

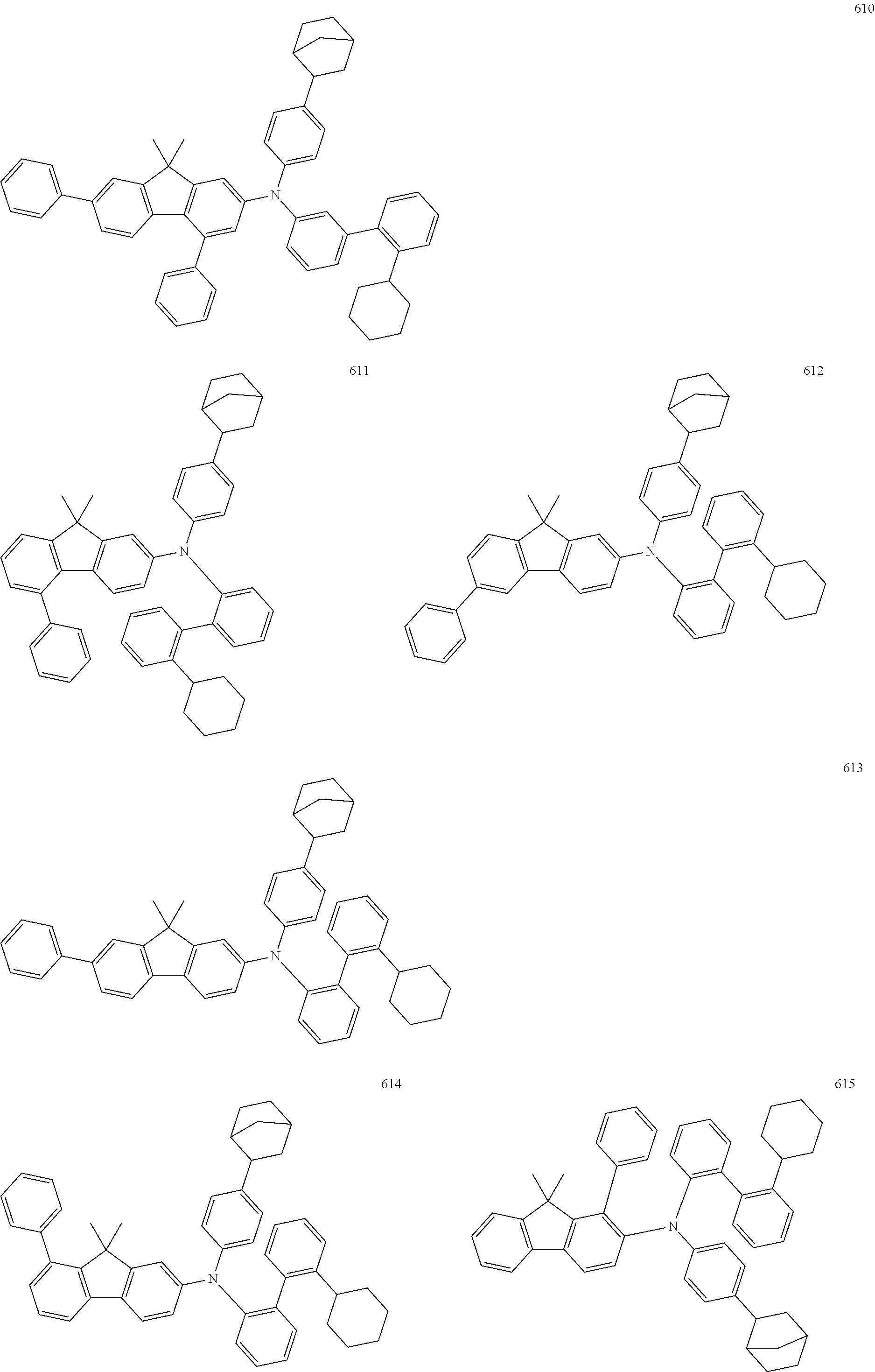

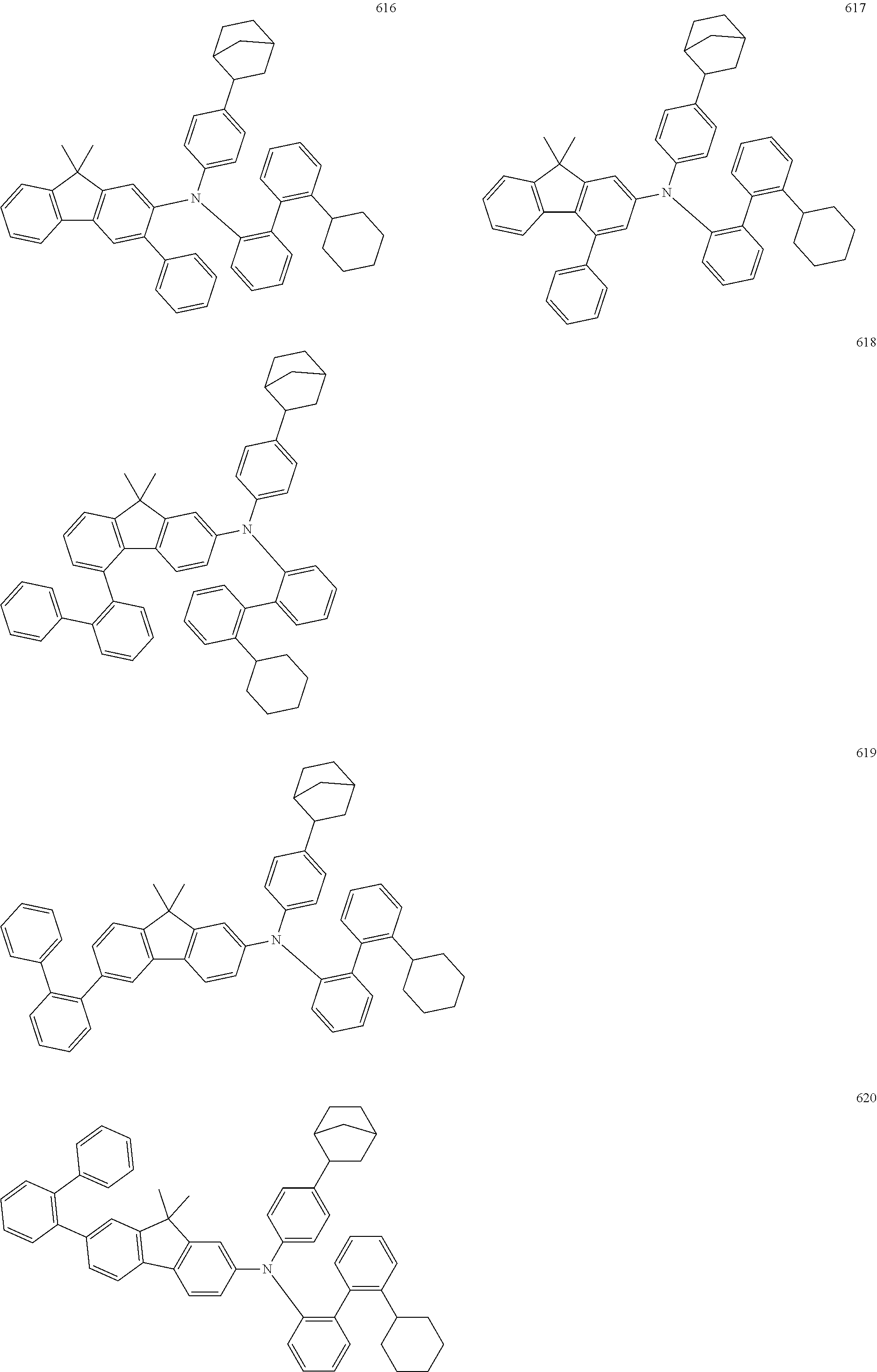 [SMILES: CC1(C)c2ccccc2-c2cc(-c3ccccc3)c(N(c3ccc(C4CC5CCC4C5)cc3)c3ccccc3-c3ccccc3C3CCCCC3)cc21 ; CC1(C)c2ccccc2-c2c(-c3ccccc3)cc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3ccccc3-c3ccccc3C3CCCCC3)cc21 ; CC1(C)c2cc(-c3ccccc3-c3ccccc3)ccc2-c2ccc(N(c3ccc(C4CC5CCC4C5)cc3)c3ccccc3-c3ccccc3C3CCCCC3)cc21]

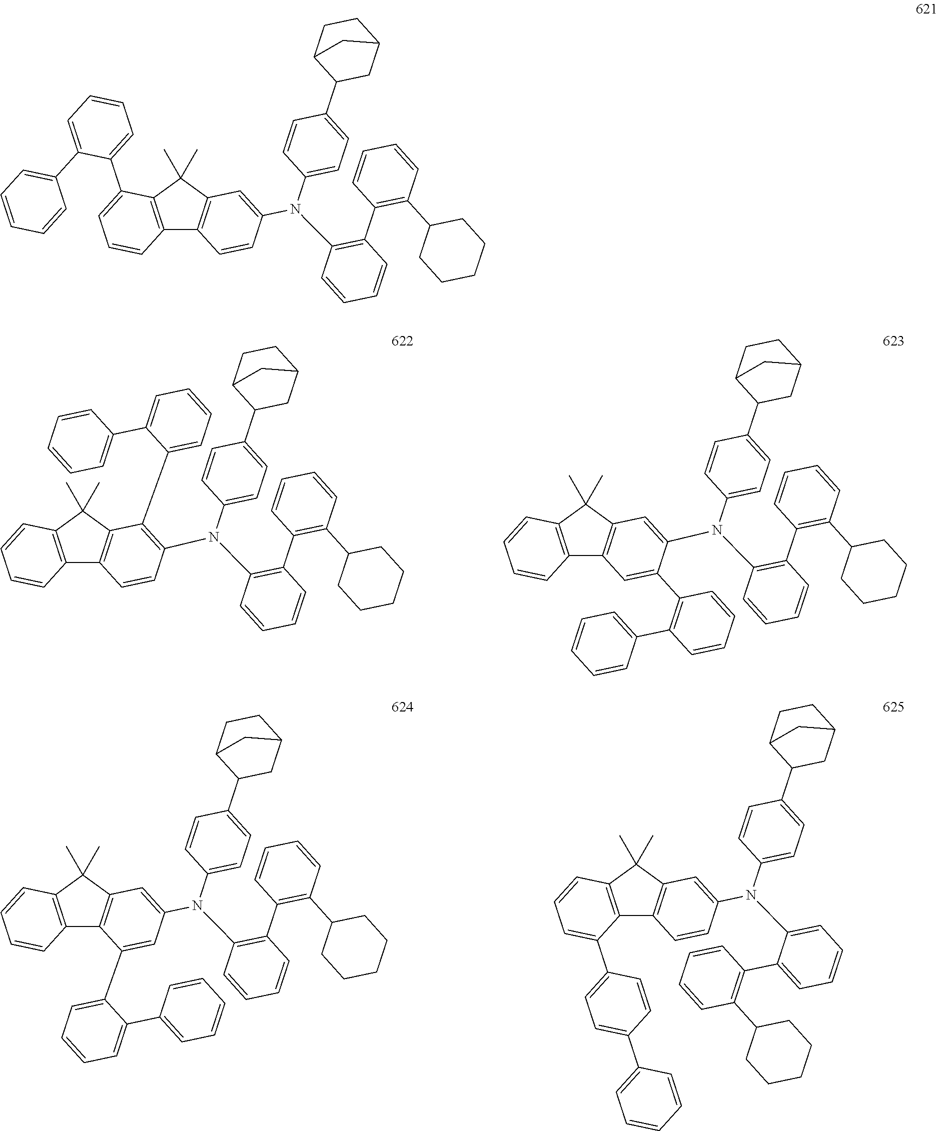 [SMILES: CC1(C)c2cc(N(c3ccc(C4CC5CCC4C5)cc3)c3ccccc3-c3ccccc3C3CCCCC3)ccc2-c2cccc(-c3ccccc3-c3ccccc3)c21 ; CC1(C)c2ccccc2-c2ccc(N(c3ccc(C4CC5CCC4C5)cc3)c3ccccc3-c3ccccc3C3CCCCC3)c(-c3ccccc3-c3ccccc3)c21 ; CC1(C)c2ccccc2-c2cc(-c3ccccc3-c3ccccc3)c(N(c3ccc(C4CC5CCC4C5)cc3)c3ccccc3-c3ccccc3C3CCCCC3)cc21 ; CC1(C)c2ccccc2-c2c(-c3ccccc3-c3ccccc3)cc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2c(-c3ccc(-c4ccccc4)cc3)cccc21]

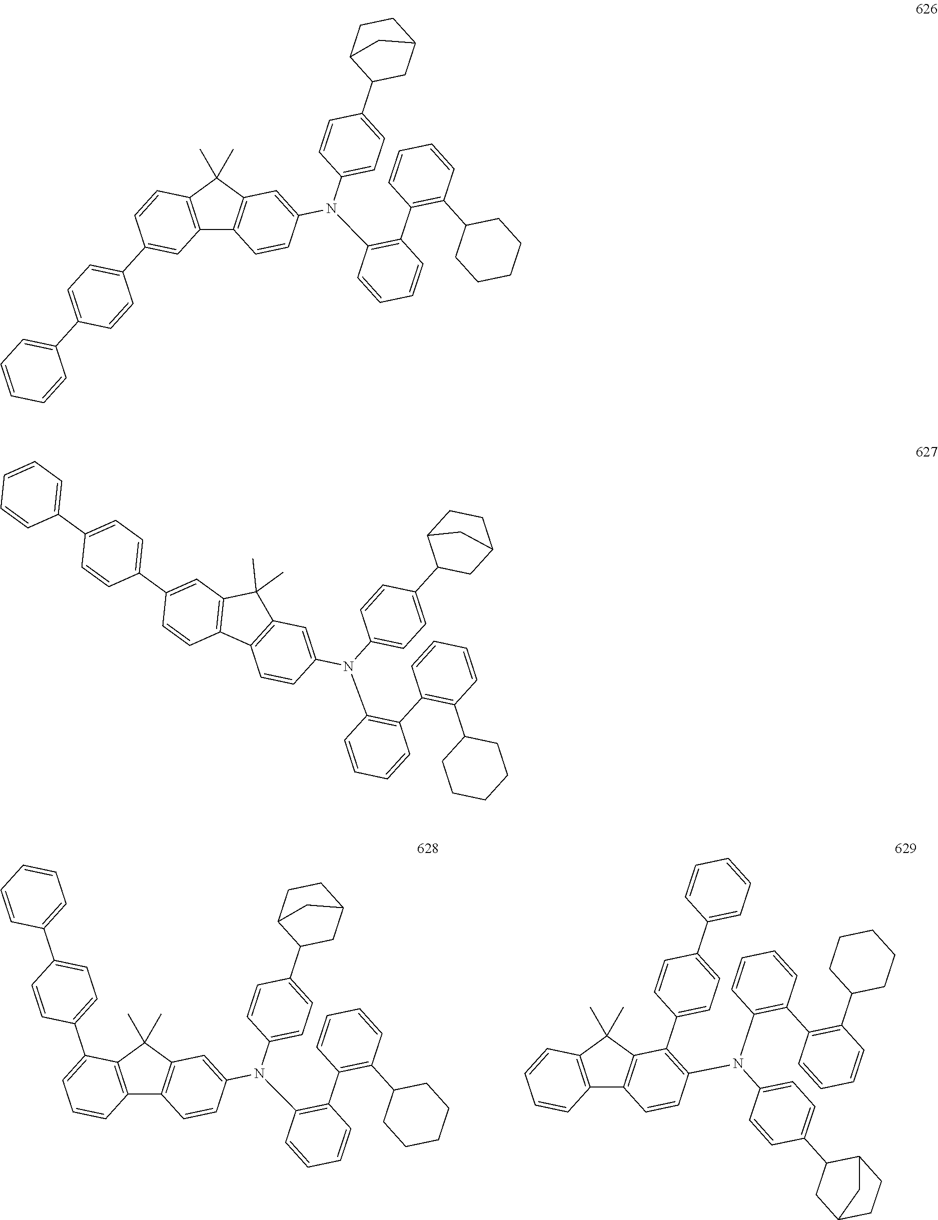 [SMILES: CC1(C)c2ccc(-c3ccc(-c4ccccc4)cc3)cc2-c2ccc(N(c3ccc(C4CC5CCC4C5)cc3)c3ccccc3-c3ccccc3C3CCCCC3)cc21 ; CC1(C)c2cc(-c3ccc(-c4ccccc4)cc3)ccc2-c2ccc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2cccc(-c3ccc(-c4ccccc4)cc3)c21 ; CC1(C)c2ccccc2-c2ccc(N(c3ccc(C4CC5CCC4C5)cc3)c3ccccc3-c3ccccc3C3CCCCC3)c(-c3ccc(-c4ccccc4)cc3)c21]

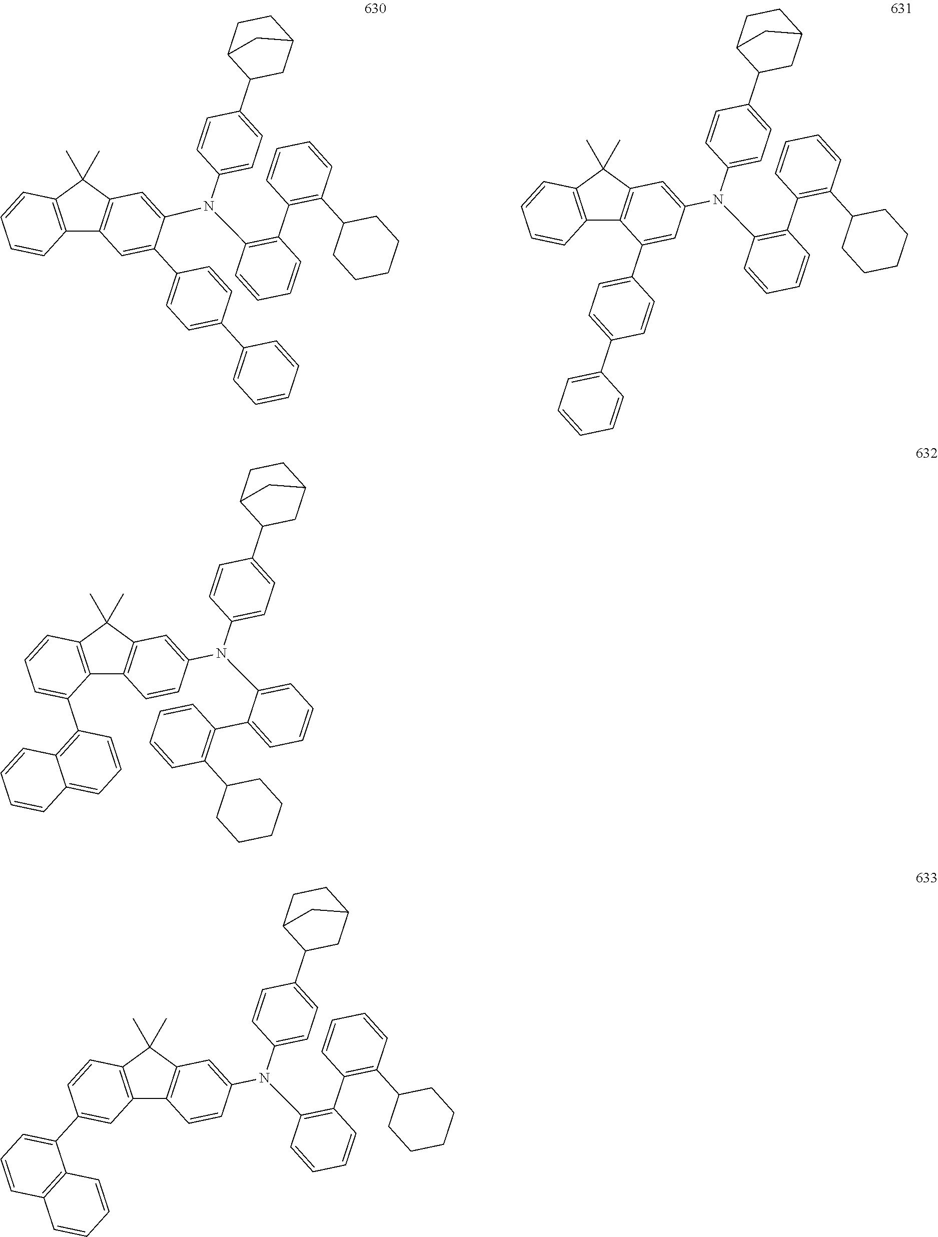 [SMILES: CC1(C)c2ccccc2-c2cc(-c3ccc(-c4ccccc4)cc3)c(N(c3ccc(C4CC5CCC4C5)cc3)c3ccccc3-c3ccccc3C3CCCCC3)cc21 ; CC1(C)c2ccccc2-c2c(-c3ccc(-c4ccccc4)cc3)cc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccccc3-c3ccccc3C3CCCCC3)cc21]

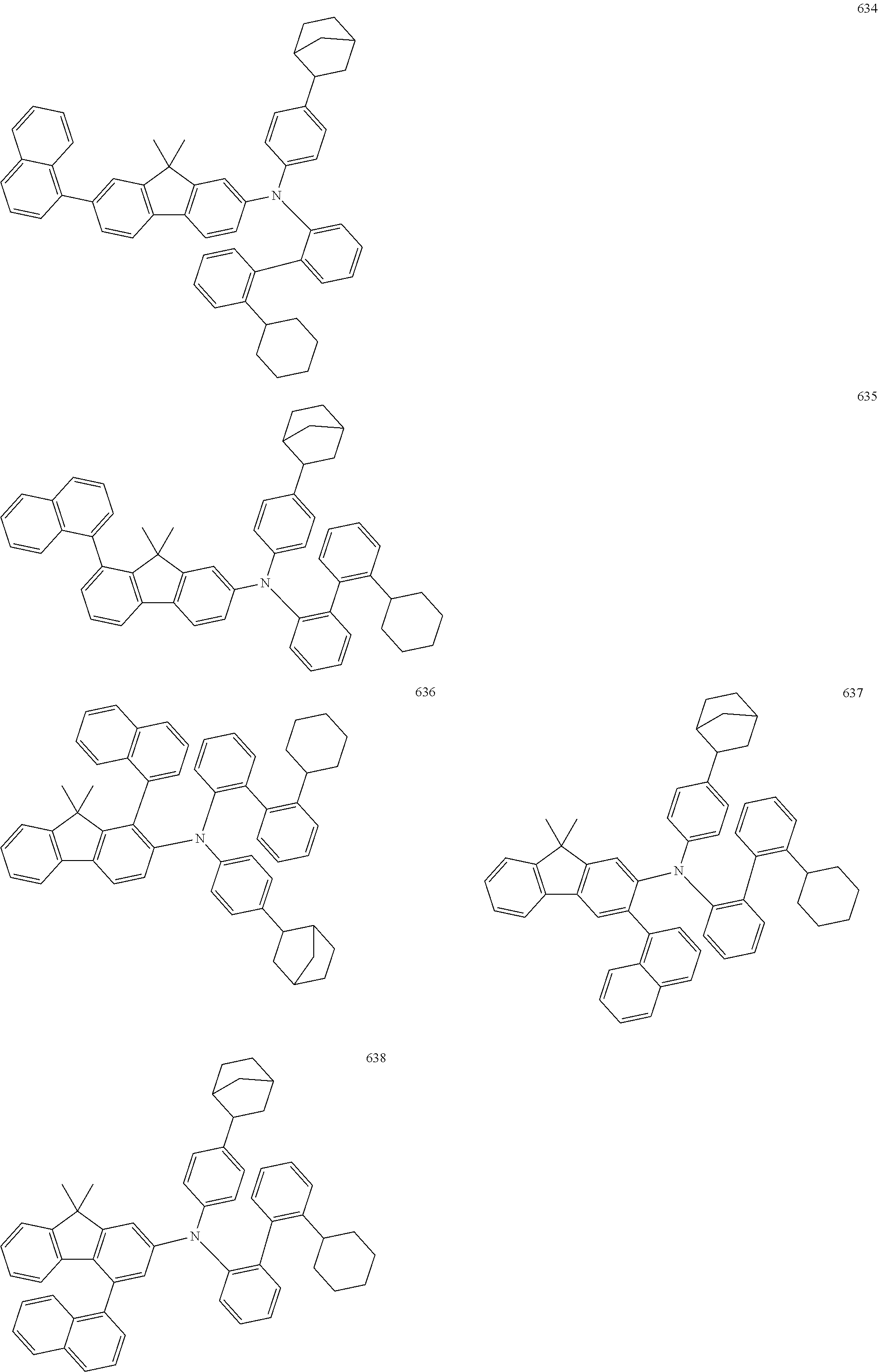

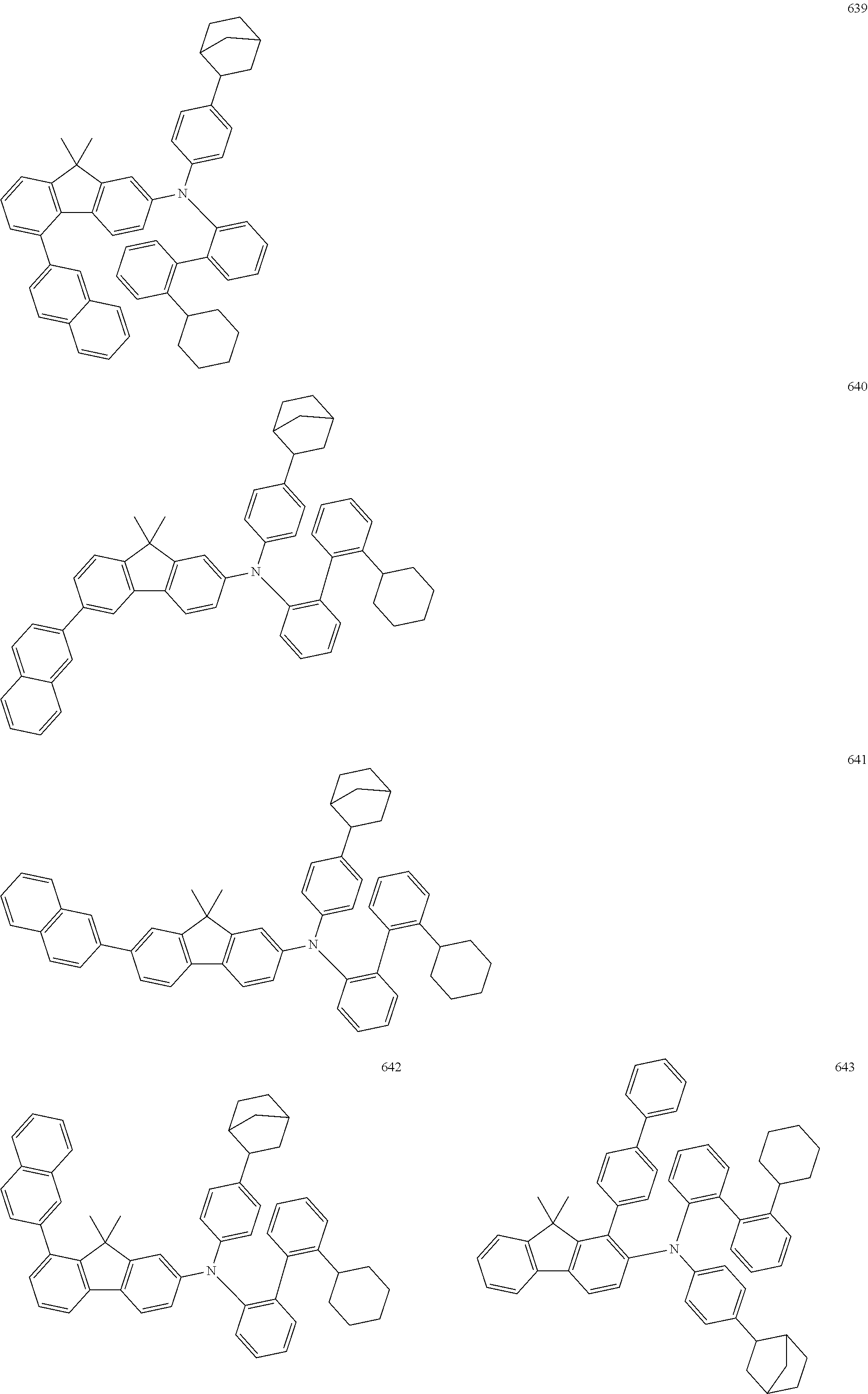 [SMILES: CC1(C)c2cc(N(c3ccc(C4CC5CCC4C5)cc3)c3ccccc3-c3ccccc3C3CCCCC3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccccc3-c3ccccc3C3CCCCC3)cc21 ; CC1(C)c2cc(-c3ccc4ccccc4c3)ccc2-c2ccc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2cccc(-c3ccc4ccccc4c3)c21 ; CC1(C)c2ccccc2-c2ccc(N(c3ccc(C4CC5CCC4C5)cc3)c3ccccc3-c3ccccc3C3CCCCC3)c(-c3ccc(-c4ccccc4)cc3)c21]

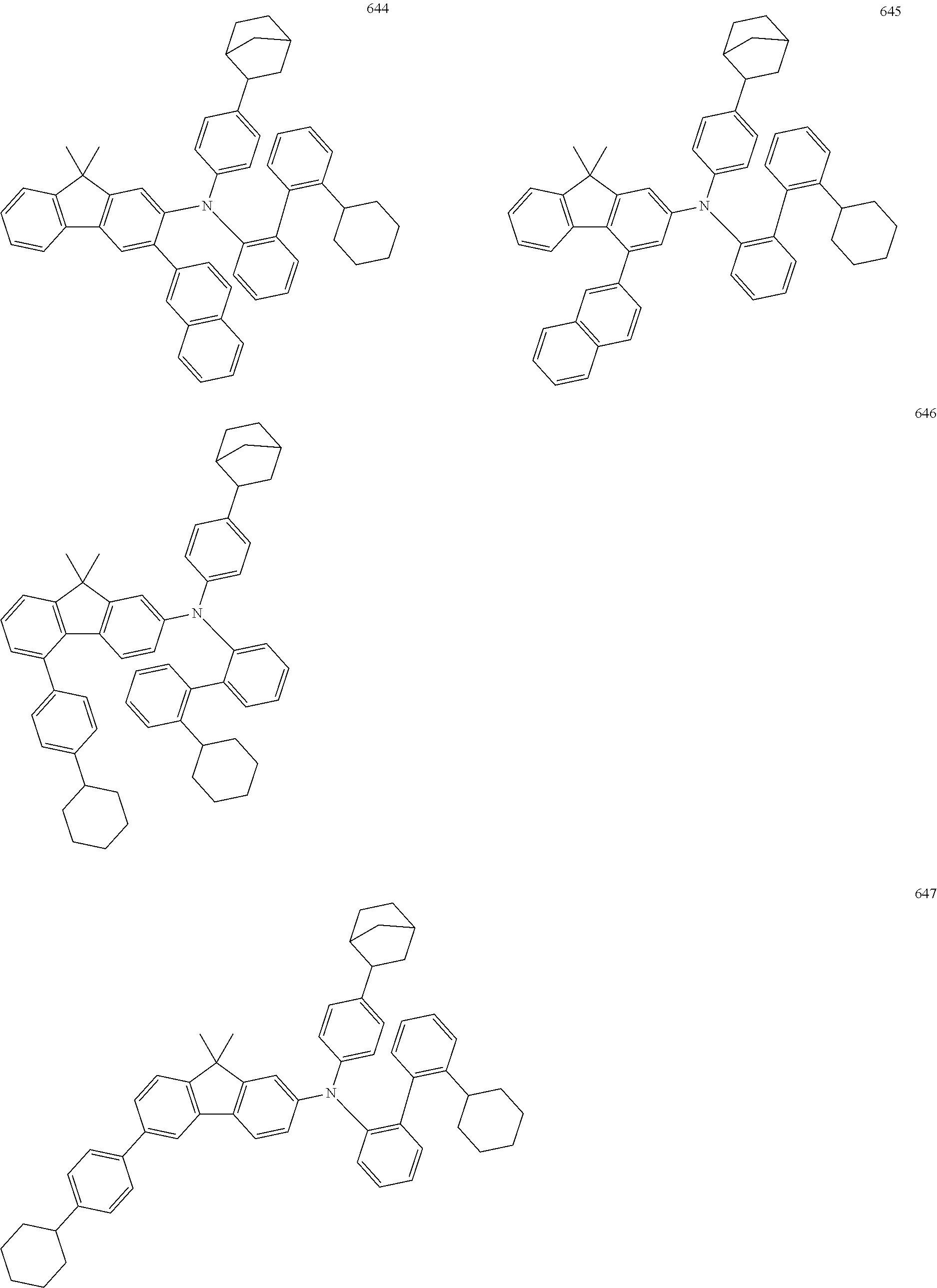 [SMILES: CC1(C)c2ccccc2-c2cc(-c3ccc4ccccc4c3)c(N(c3ccc(C4CC5CCC4C5)cc3)c3ccccc3-c3ccccc3C3CCCCC3)cc21 ; CC1(C)c2ccccc2-c2c(-c3ccc4ccccc4c3)cc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3ccccc3-c3ccccc3C3CCCCC3)cc21]

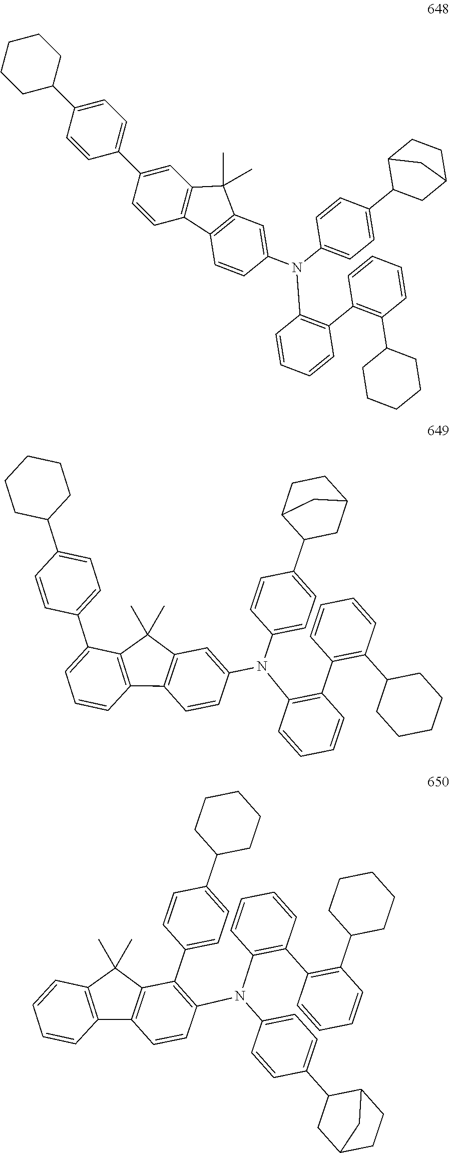 [SMILES: CC1(C)c2cc(-c3ccc(C4CCCCC4)cc3)ccc2-c2ccc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2cccc(-c3ccc(C4CCCCC4)cc3)c21 ; CC1(C)c2ccccc2-c2ccc(N(c3ccc(C4CC5CCC4C5)cc3)c3ccccc3-c3ccccc3C3CCCCC3)c(-c3ccc(C4CCCCC4)cc3)c21]

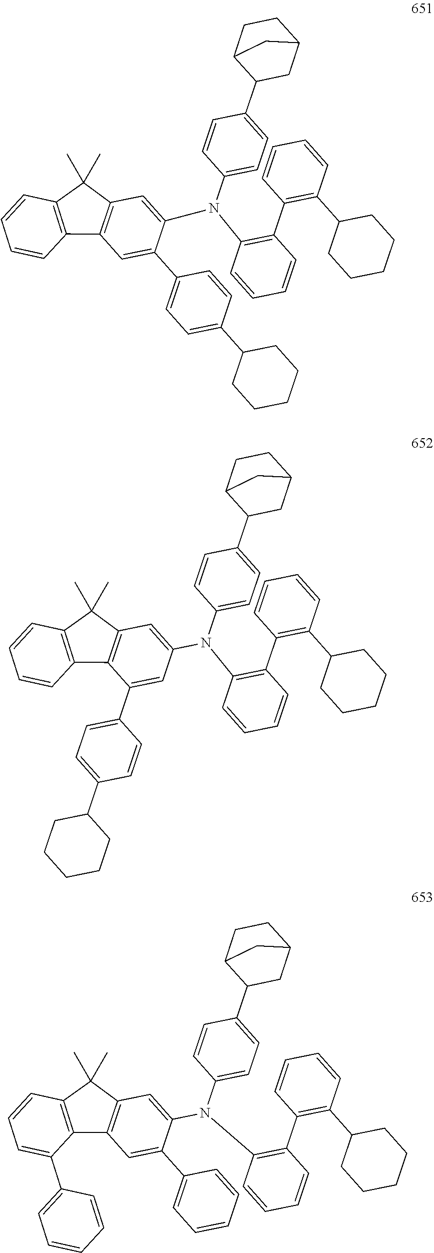

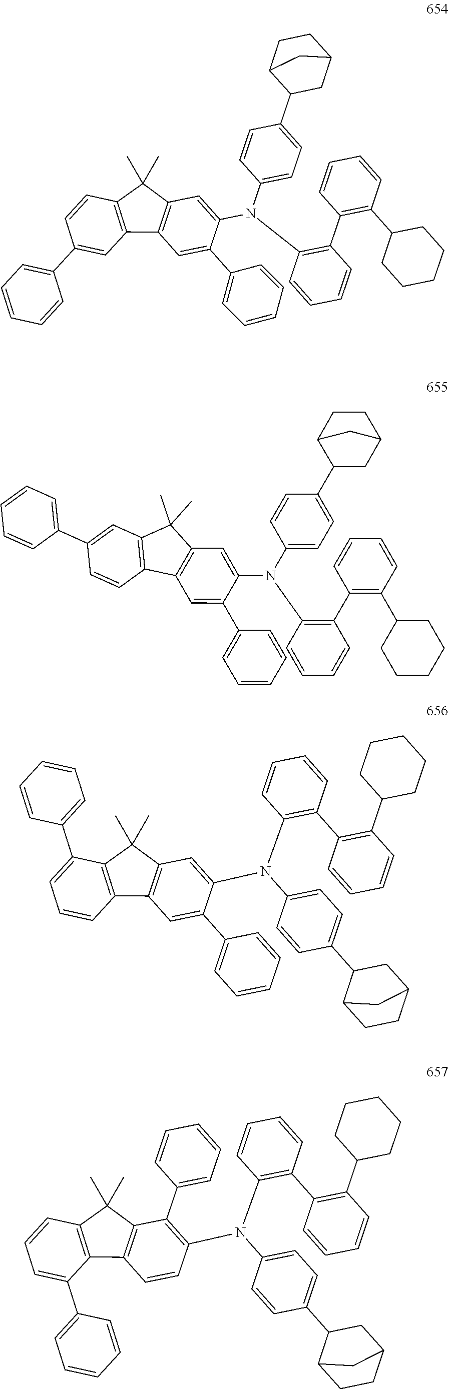 [SMILES: CC1(C)c2ccc(-c3ccccc3)cc2-c2cc(-c3ccccc3)c(N(c3ccc(C4CC5CCC4C5)cc3)c3ccccc3-c3ccccc3C3CCCCC3)cc21 ; CC1(C)c2cc(-c3ccccc3)ccc2-c2cc(-c3ccccc3)c(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3ccccc3-c3ccccc3C3CCCCC3)c(-c3ccccc3)c21]

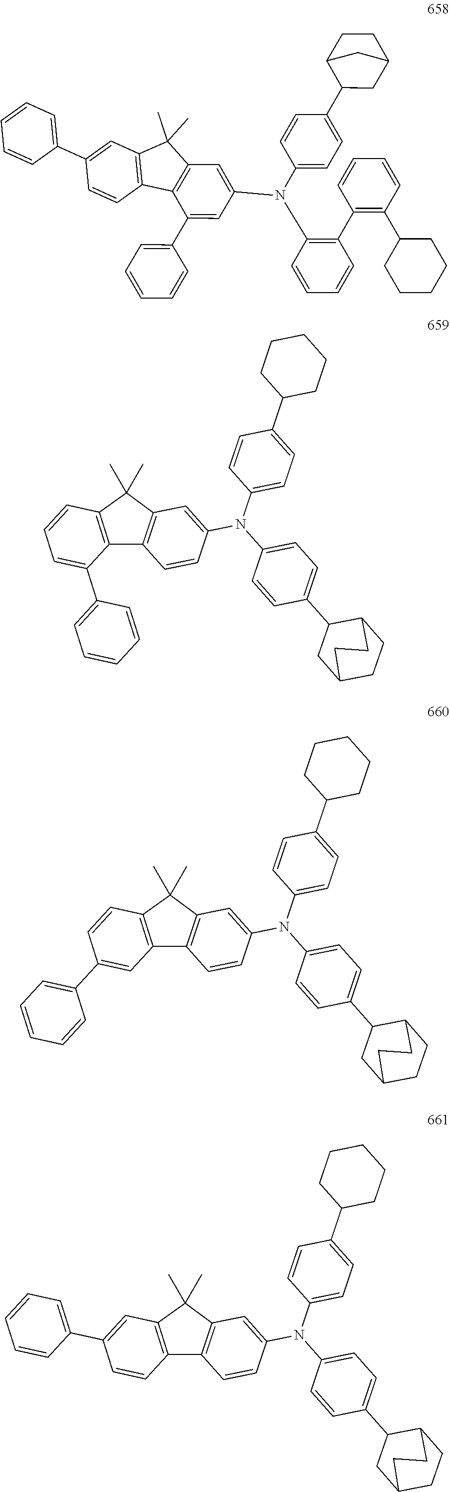

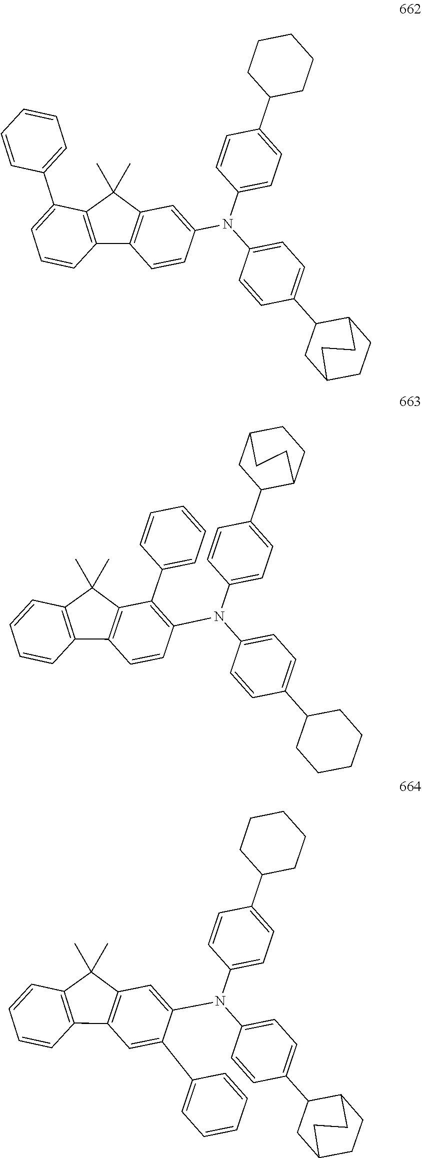 [SMILES: CC1(C)c2cc(N(c3ccc(C4CCCCC4)cc3)c3ccc(C4CC5CCC4CC5)cc3)ccc2-c2cccc(-c3ccccc3)c21 ; CC1(C)c2ccccc2-c2ccc(N(c3ccc(C4CCCCC4)cc3)c3ccc(C4CC5CCC4CC5)cc3)c(-c3ccccc3)c21 ; CC1(C)c2ccccc2-c2cc(-c3ccccc3)c(N(c3ccc(C4CCCCC4)cc3)c3ccc(C4CC5CCC4CC5)cc3)cc21]

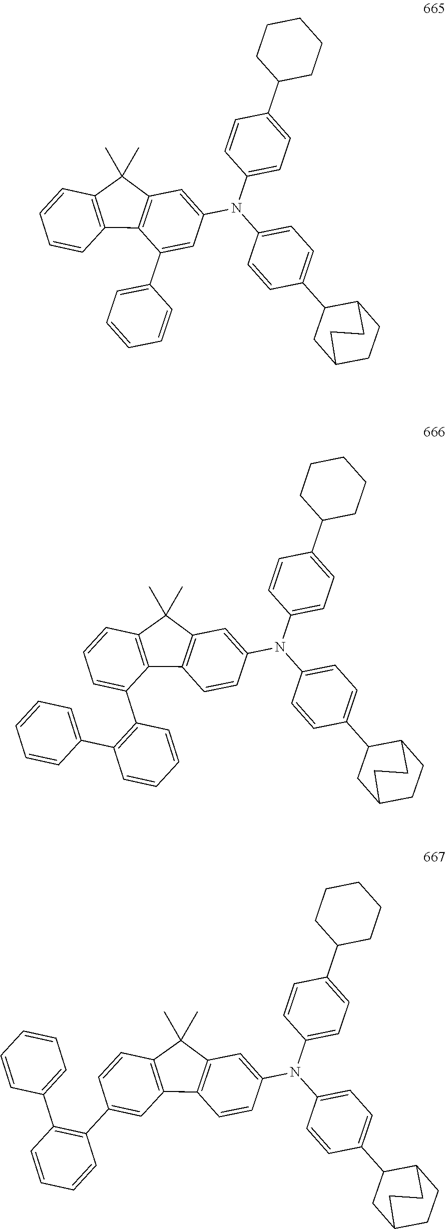 [SMILES: CC1(C)c2ccccc2-c2c(-c3ccccc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4CC5CCC4CC5)cc3)cc21]

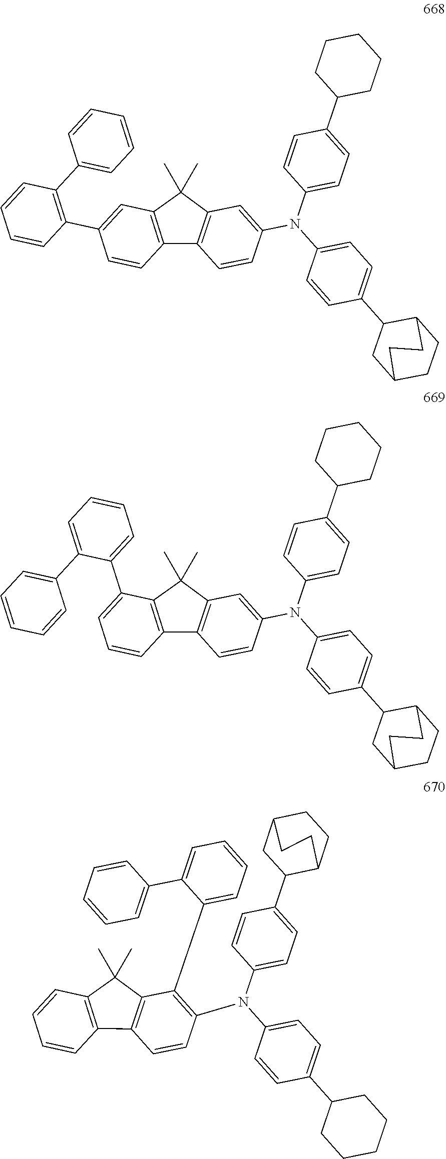 [SMILES: CC1(C)c2cc(-c3ccccc3-c3ccccc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4CC5)cc3)c(-c3ccccc3-c3ccccc3)c21]

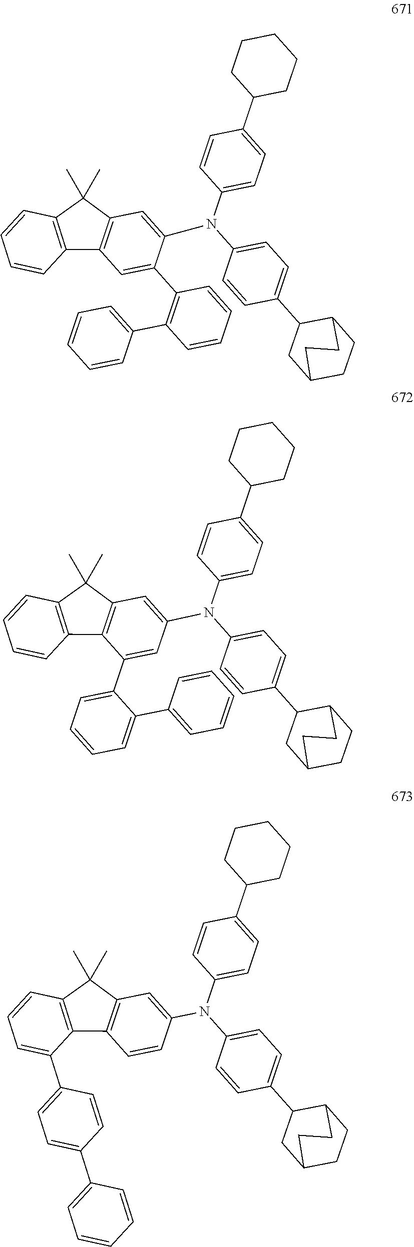 [SMILES: CC1(C)c2ccccc2-c2cc(-c3ccccc3-c3ccccc3)c(N(c3ccc(C4CCCCC4)cc3)c3ccc(C4CC5CCC4CC5)cc3)cc21 ; CC1(C)c2ccccc2-c2c(-c3ccccc3-c3ccccc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc(-c4ccccc4)cc3)cccc21]

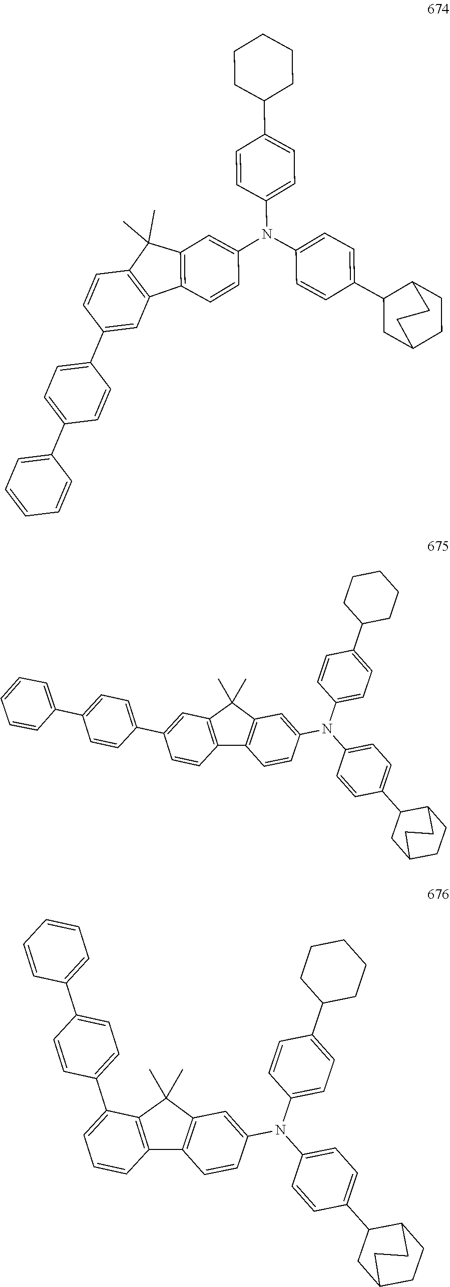 [SMILES: CC1(C)c2ccc(-c3ccc(-c4ccccc4)cc3)cc2-c2ccc(N(c3ccc(C4CCCCC4)cc3)c3ccc(C4CC5CCC4CC5)cc3)cc21 ; CC1(C)c2cc(-c3ccc(-c4ccccc4)cc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc(-c4ccccc4)cc3)c21]

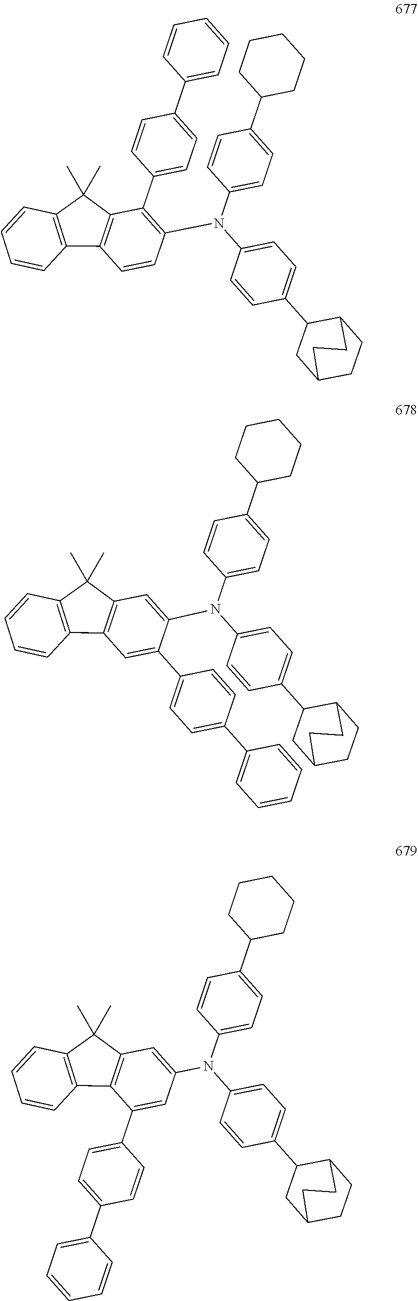 [SMILES: CC1(C)c2ccccc2-c2ccc(N(c3ccc(C4CCCCC4)cc3)c3ccc(C4CC5CCC4CC5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4CC5CCC4CC5)cc3)cc21 ; CC1(C)c2ccccc2-c2c(-c3ccc(-c4ccccc4)cc3)cc(N(c3ccc(C4CCCCC4)cc3)c3ccc(C4CC5CCC4CC5)cc3)cc21]

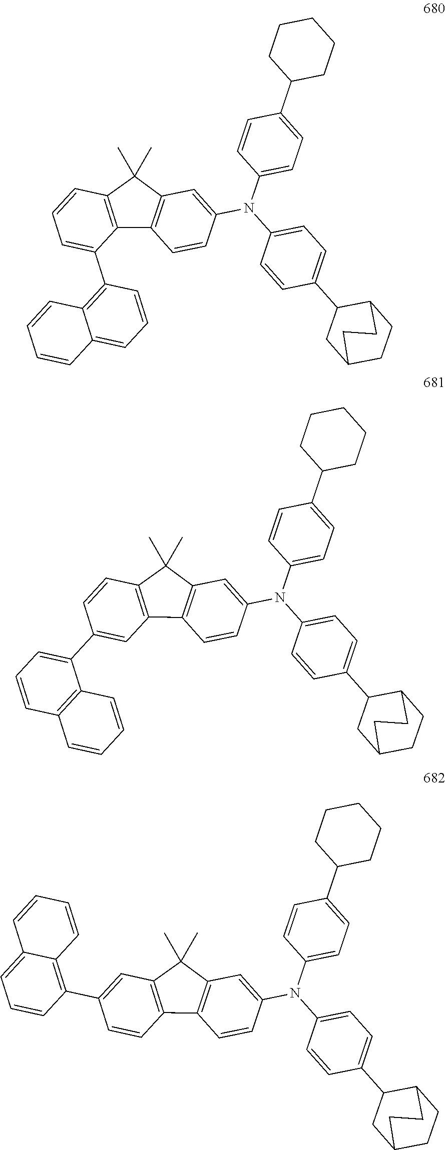

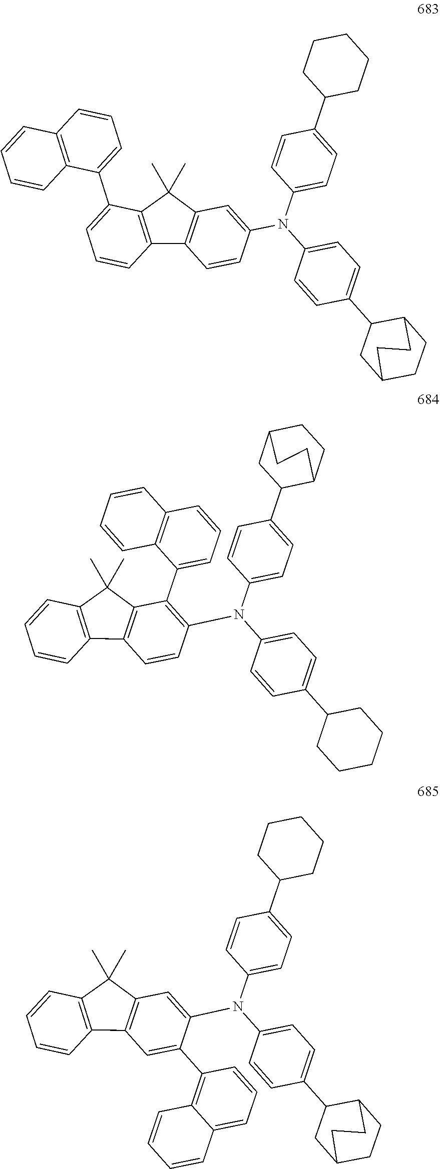 [SMILES: CC1(C)c2cc(N(c3ccc(C4CCCCC4)cc3)c3ccc(C4CC5CCC4CC5)cc3)ccc2-c2cccc(-c3cccc4ccccc34)c21 ; CC1(C)c2ccccc2-c2ccc(N(c3ccc(C4CCCCC4)cc3)c3ccc(C4CC5CCC4CC5)cc3)c(-c3cccc4ccccc34)c21 ; CC1(C)c2ccccc2-c2cc(-c3cccc4ccccc34)c(N(c3ccc(C4CCCCC4)cc3)c3ccc(C4CC5CCC4CC5)cc3)cc21]

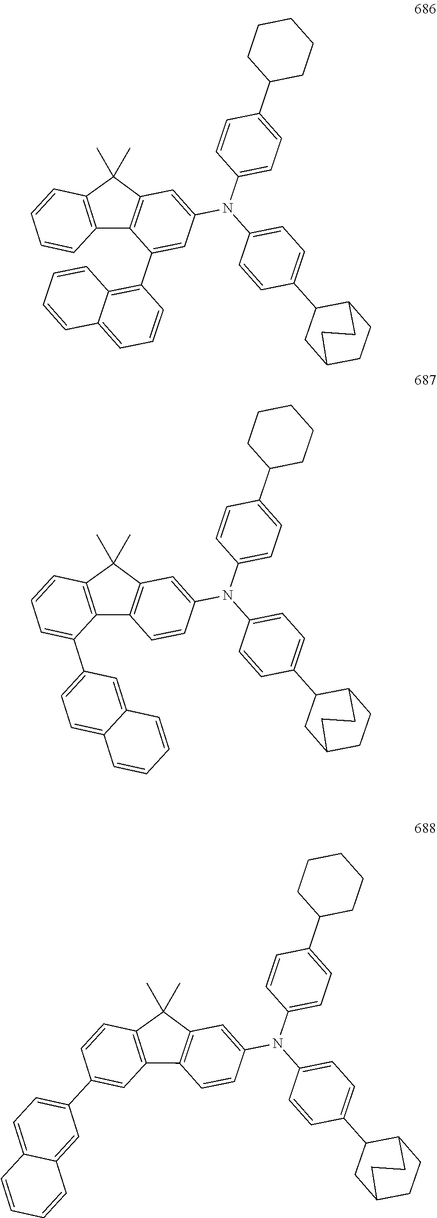 [SMILES: CC1(C)c2ccccc2-c2c(-c3cccc4ccccc34)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CC5CCC4CC5)cc3)cc21]

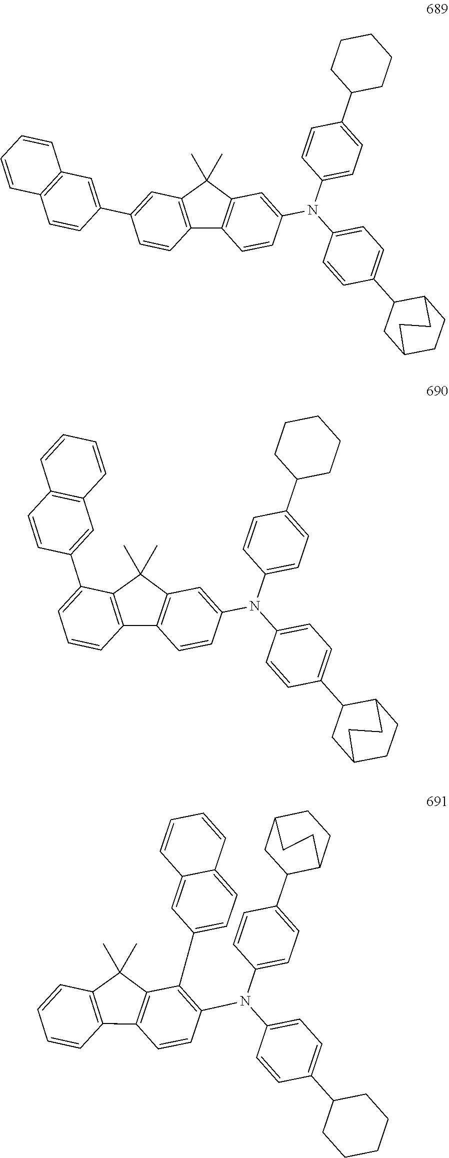 [SMILES: CC1(C)c2cc(-c3ccc4ccccc4c3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc4ccccc4c3)c21 ; CC1(C)c2ccccc2-c2ccc(N(c3ccc(C4CCCCC4)cc3)c3ccc(C4CC5CCC4CC5)cc3)c(-c3ccc4ccccc4c3)c21]

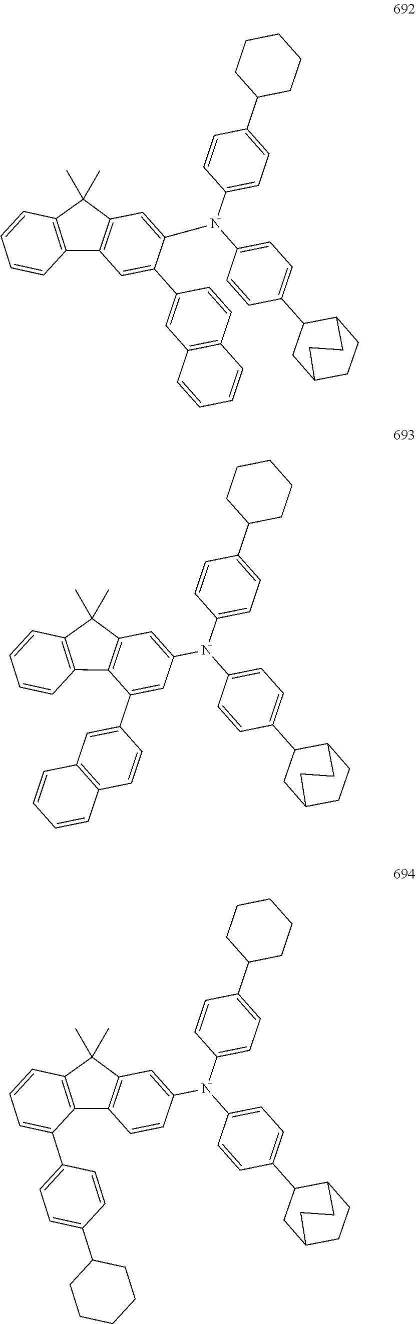 [SMILES: CC1(C)c2ccccc2-c2cc(-c3ccc4ccccc4c3)c(N(c3ccc(C4CCCCC4)cc3)c3ccc(C4CC5CCC4CC5)cc3)cc21 ; CC1(C)c2ccccc2-c2c(-c3ccc4ccccc4c3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc(C4CCCCC4)cc3)cccc21]

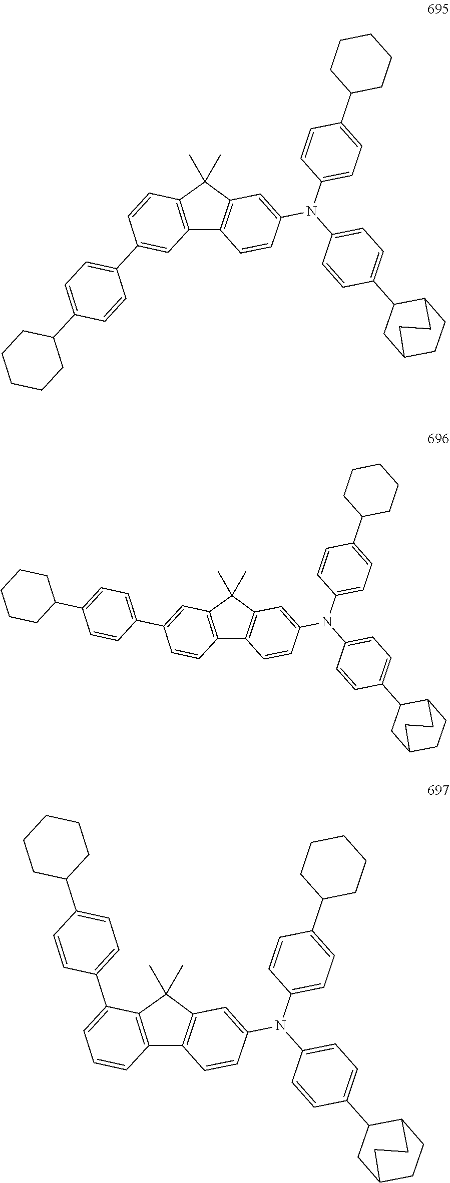

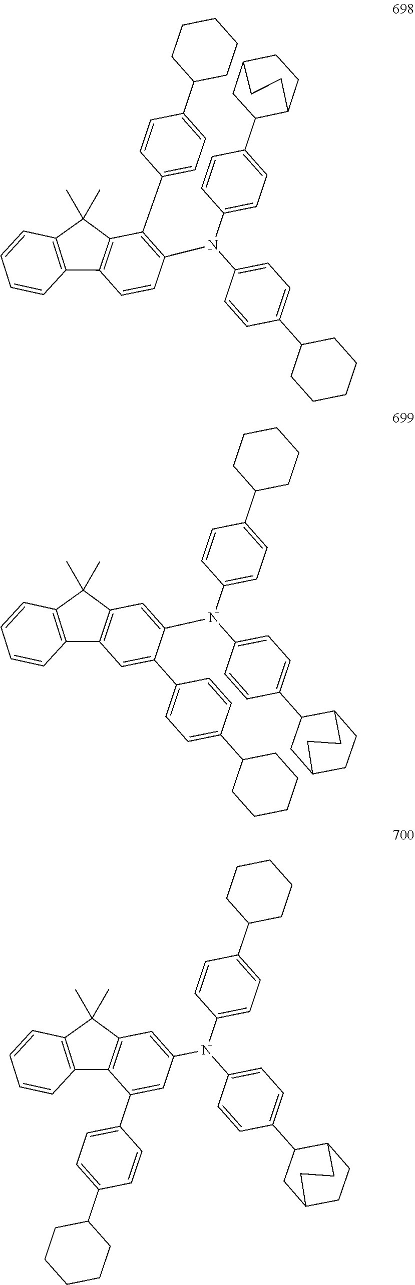

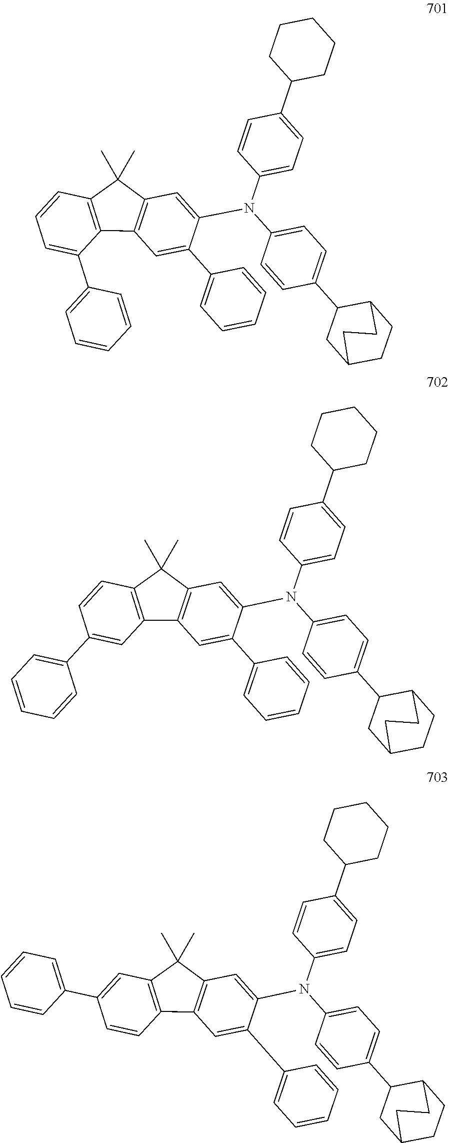

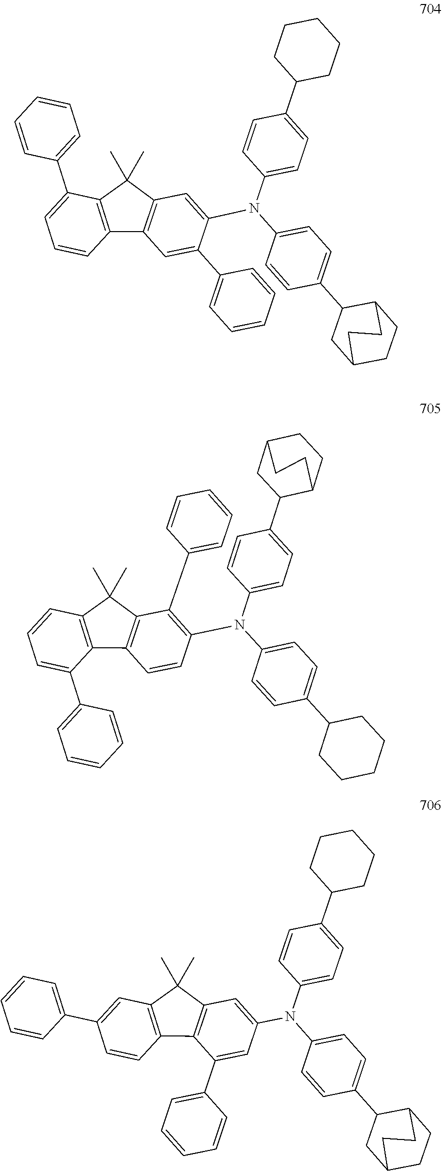

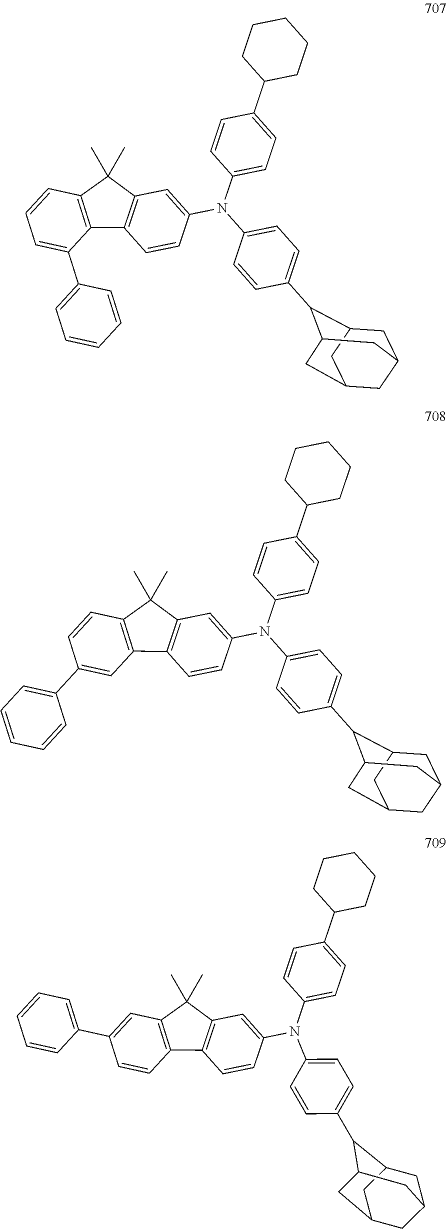

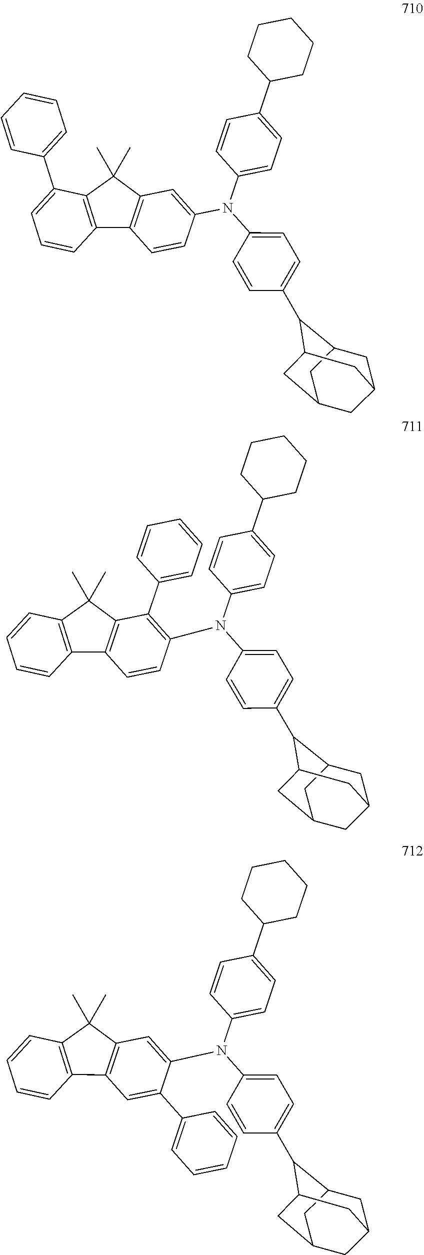 [SMILES: CC1(C)c2cc(N(c3ccc(C4CCCCC4)cc3)c3ccc(C4C5CC6CC(C5)CC4C6)cc3)ccc2-c2cccc(-c3ccccc3)c21 ; CC1(C)c2ccccc2-c2ccc(N(c3ccc(C4CCCCC4)cc3)c3ccc(C4C5CC6CC(C5)CC4C6)cc3)c(-c3ccccc3)c21 ; CC1(C)c2ccccc2-c2cc(-c3ccccc3)c(N(c3ccc(C4CCCCC4)cc3)c3ccc(C4C5CC6CC(C5)CC4C6)cc3)cc21]

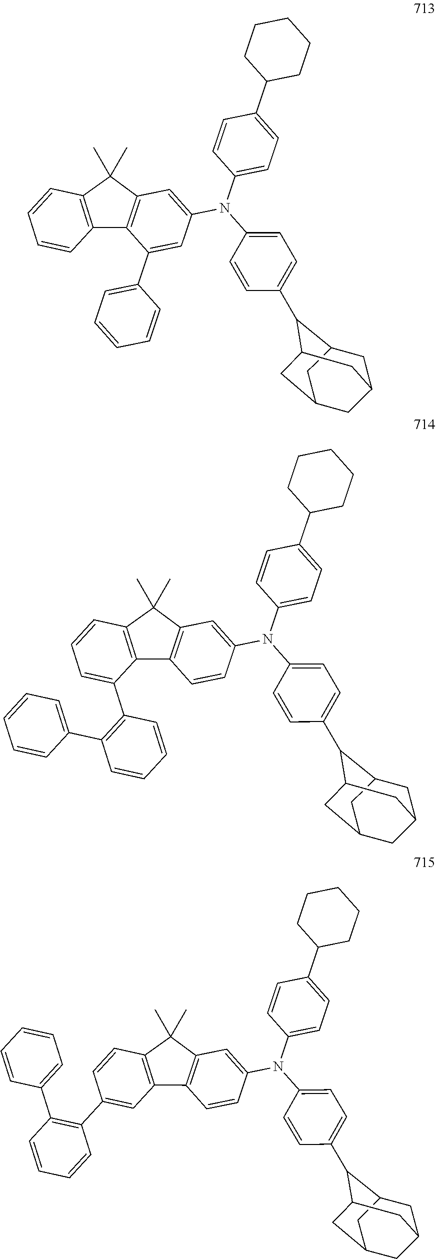

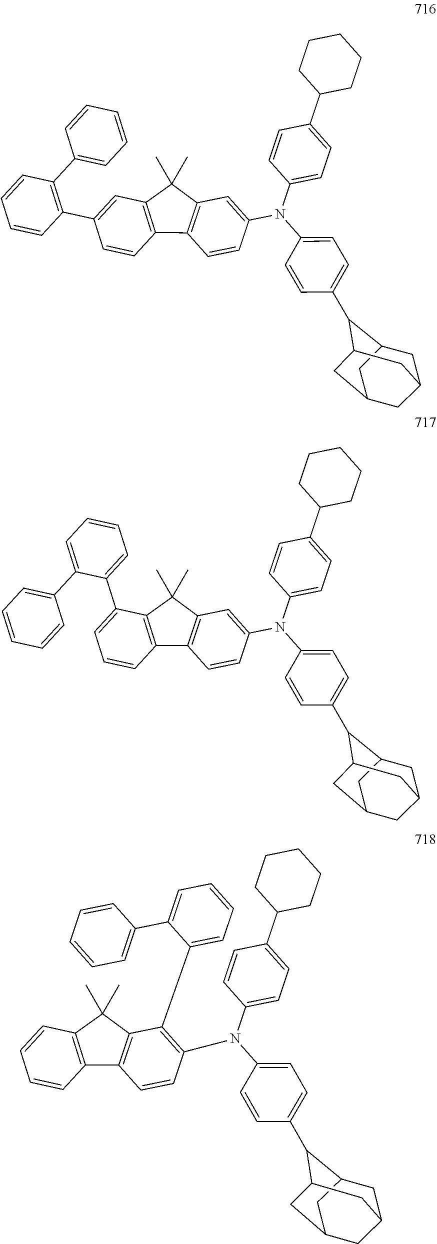 [SMILES: CC1(C)c2cc(-c3ccccc3-c3ccccc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccccc3-c3ccccc3)c21 ; CC1(C)c2ccccc2-c2ccc(N(c3ccc(C4CCCCC4)cc3)c3ccc(C4C5CC6CC(C5)CC4C6)cc3)c(-c3ccccc3-c3ccccc3)c21]

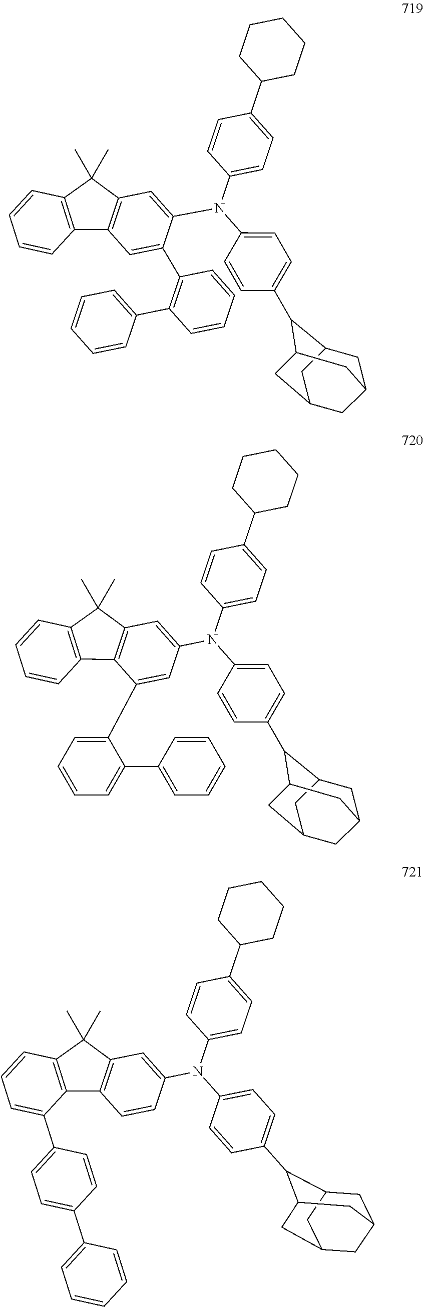 [SMILES: CC1(C)c2ccccc2-c2cc(-c3ccccc3-c3ccccc3)c(N(c3ccc(C4CCCCC4)cc3)c3ccc(C4C5CC6CC(C5)CC4C6)cc3)cc21 ; CC1(C)c2ccccc2-c2c(-c3ccccc3-c3ccccc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc(-c4ccccc4)cc3)cccc21]

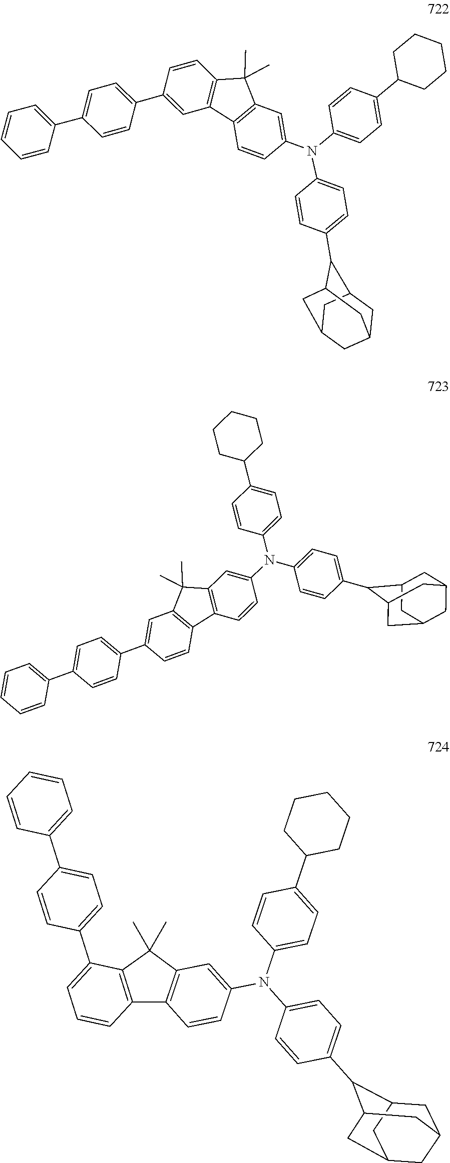 [SMILES: CC1(C)c2ccc(-c3ccc(-c4ccccc4)cc3)cc2-c2ccc(N(c3ccc(C4CCCCC4)cc3)c3ccc(C4C5CC6CC(C5)CC4C6)cc3)cc21 ; CC1(C)c2cc(-c3ccc(-c4ccccc4)cc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc(-c4ccccc4)cc3)c21]

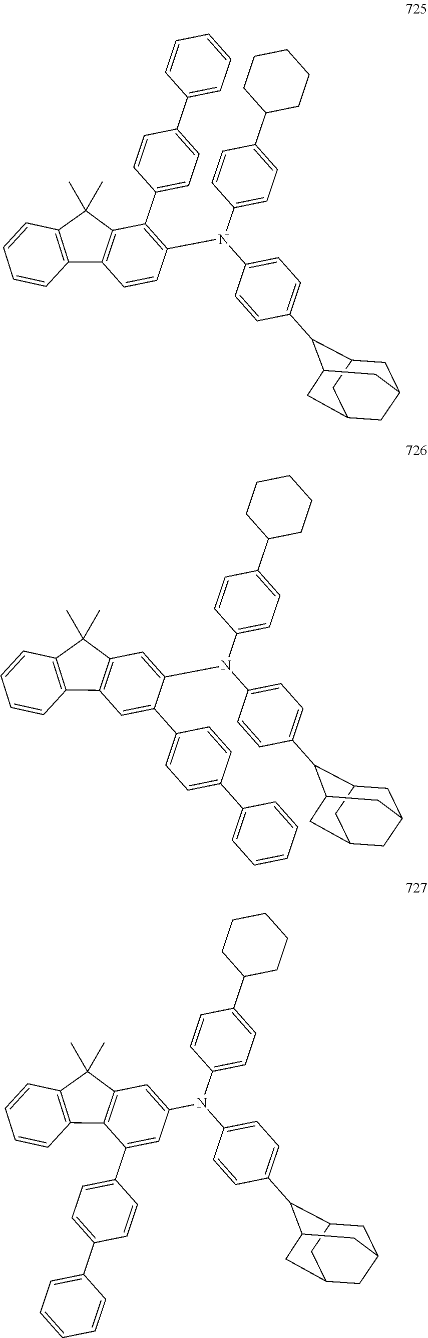 [SMILES: CC1(C)c2ccccc2-c2ccc(N(c3ccc(C4CCCCC4)cc3)c3ccc(C4C5CC6CC(C5)CC4C6)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4C5CC6CC(C5)CC4C6)cc3)cc21 ; CC1(C)c2ccccc2-c2c(-c3ccc(-c4ccccc4)cc3)cc(N(c3ccc(C4CCCCC4)cc3)c3ccc(C4C5CC6CC(C5)CC4C6)cc3)cc21]

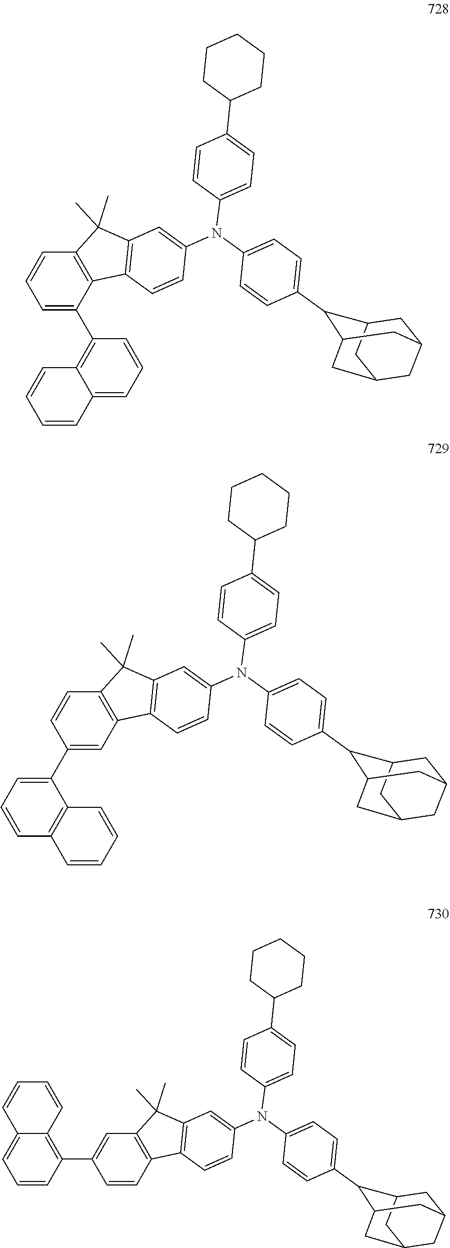 [SMILES: CC1(C)c2cc(N(c3ccc(C4CCCCC4)cc3)c3ccc(C4C5CC6CC(C5)CC4C6)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4C5CC6CC(C5)CC4C6)cc3)cc21 ; CC1(C)c2cc(-c3cccc4ccccc34)ccc2-c2ccc(N(c3ccc(C4CCCCC4)cc3)c3ccc(C4C5CC6CC(C5)CC4C6)cc3)cc21]

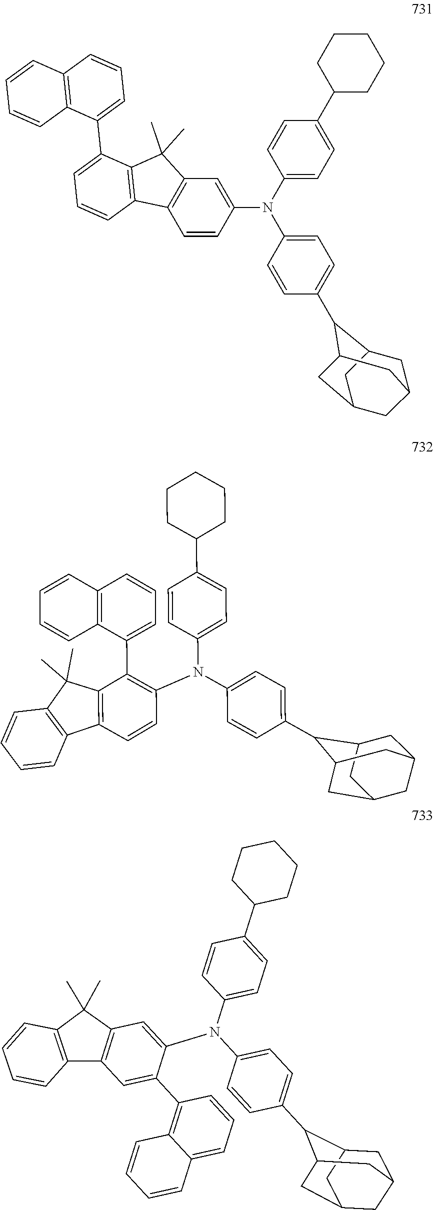 [SMILES: CC1(C)c2cc(N(c3ccc(C4CCCCC4)cc3)c3ccc(C4C5CC6CC(C5)CC4C6)cc3)ccc2-c2cccc(-c3cccc4ccccc34)c21 ; CC1(C)c2ccccc2-c2ccc(N(c3ccc(C4CCCCC4)cc3)c3ccc(C4C5CC6CC(C5)CC4C6)cc3)c(-c3cccc4ccccc34)c21 ; CC1(C)c2ccccc2-c2cc(-c3cccc4ccccc34)c(N(c3ccc(C4CCCCC4)cc3)c3ccc(C4C5CC6CC(C5)CC4C6)cc3)cc21]

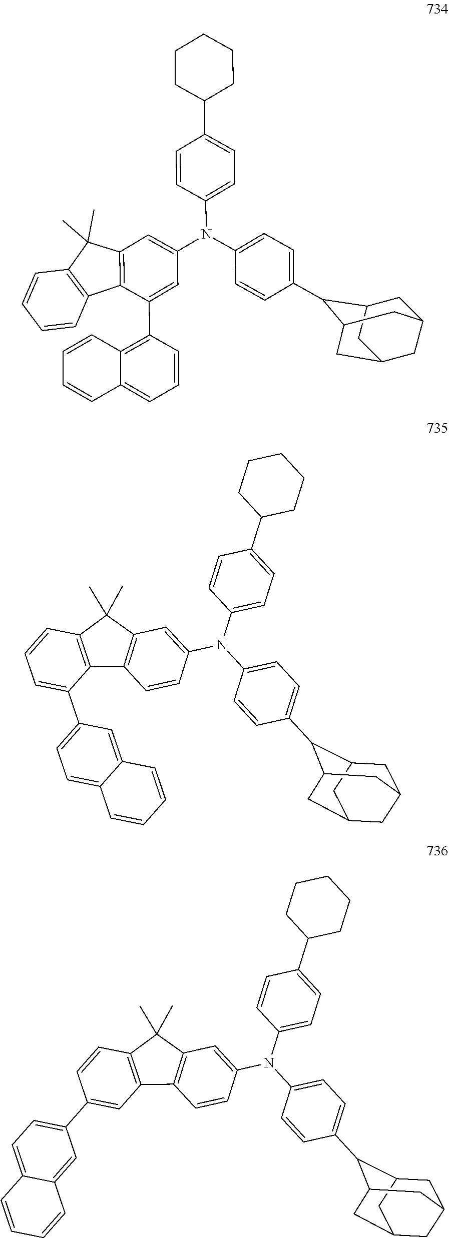 [SMILES: CC1(C)c2ccccc2-c2c(-c3cccc4ccccc34)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4C5CC6CC(C5)CC4C6)cc3)cc21]

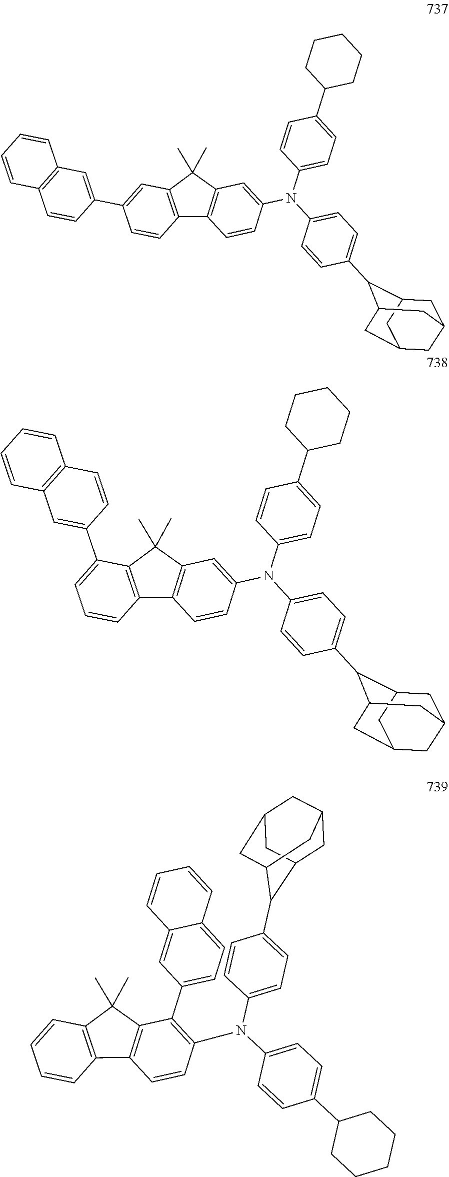 [SMILES: CC1(C)c2cc(-c3ccc4ccccc4c3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc4ccccc4c3)c21 ; CC1(C)c2ccccc2-c2ccc(N(c3ccc(C4CCCCC4)cc3)c3ccc(C4C5CC6CC(C5)CC4C6)cc3)c(-c3ccc4ccccc4c3)c21]

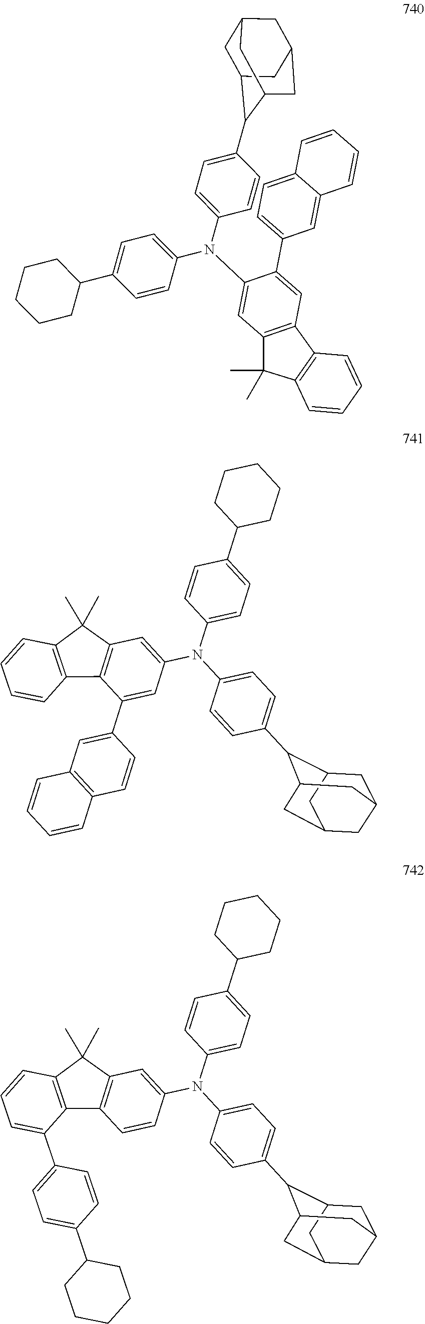 [SMILES: CC1(C)c2ccccc2-c2cc(-c3ccc4ccccc4c3)c(N(c3ccc(C4CCCCC4)cc3)c3ccc(C4C5CC6CC(C5)CC4C6)cc3)cc21 ; CC1(C)c2ccccc2-c2c(-c3ccc4ccccc4c3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc(C4CCCCC4)cc3)cccc21]

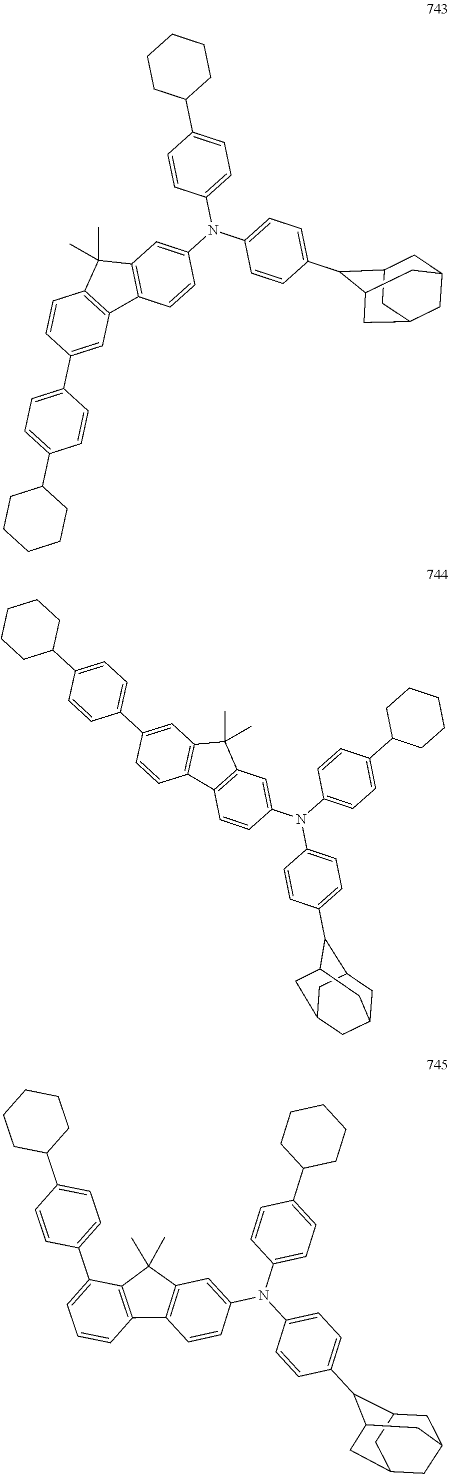 [SMILES: CC1(C)c2ccc(-c3ccc(C4CCCCC4)cc3)cc2-c2ccc(N(c3ccc(C4CCCCC4)cc3)c3ccc(C4C5CC6CC(C5)CC4C6)cc3)cc21 ; CC1(C)c2cc(-c3ccc(C4CCCCC4)cc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc(C4CCCCC4)cc3)c21]

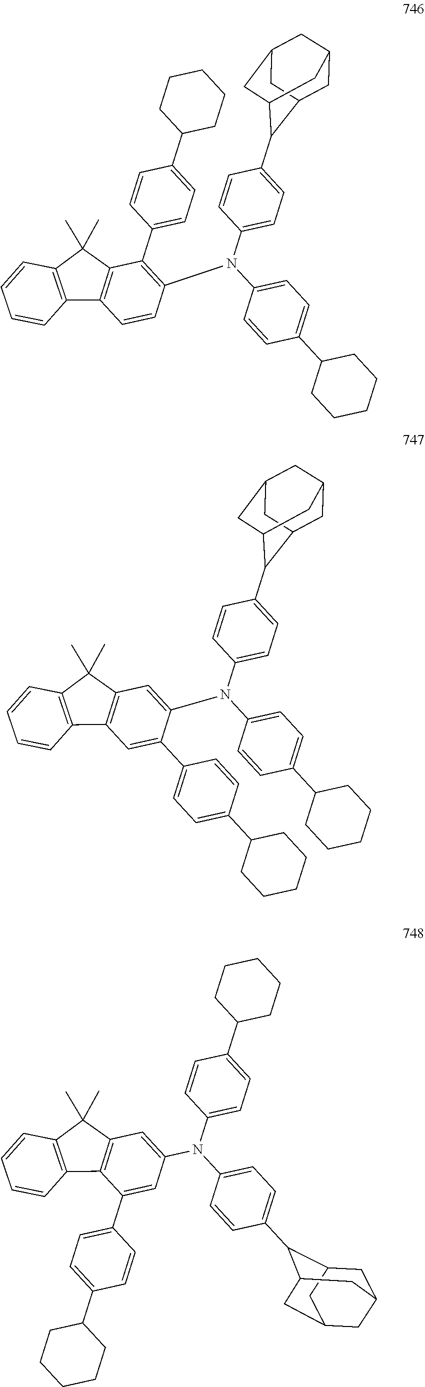 [SMILES: CC1(C)c2ccccc2-c2ccc(N(c3ccc(C4CCCCC4)cc3)c3ccc(C4C5CC6CC(C5)CC4C6)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C4C5CC6CC(C5)CC4C6)cc3)cc21 ; CC1(C)c2ccccc2-c2c(-c3ccc(C4CCCCC4)cc3)cc(N(c3ccc(C4CCCCC4)cc3)c3ccc(C4C5CC6CC(C5)CC4C6)cc3)cc21]

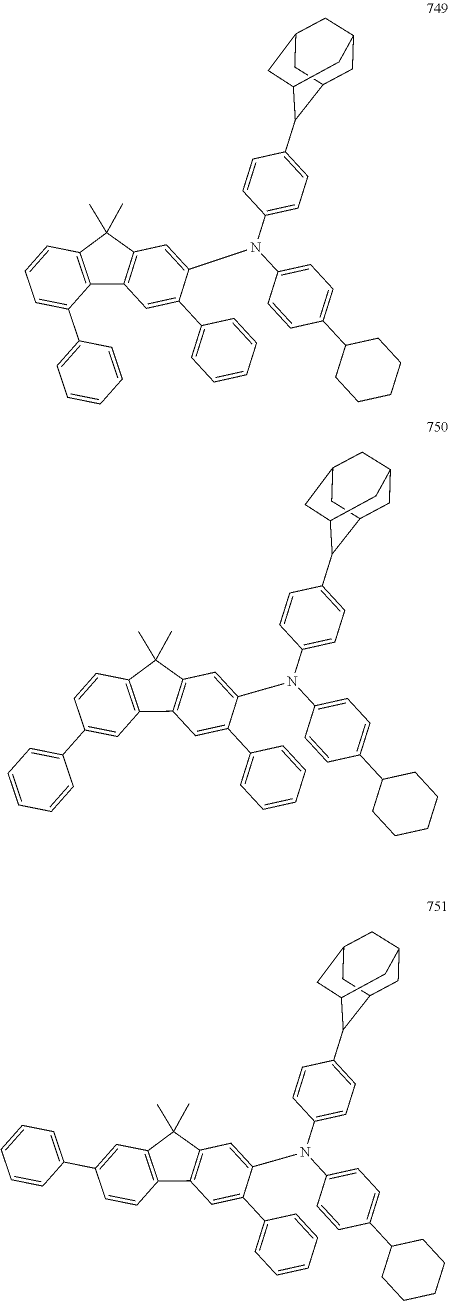 [SMILES: CC1(C)c2cc(N(c3ccc(C4CCCCC4)cc3)c3ccc(C4C5CC6CC(C5)CC4C6)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4C5CC6CC(C5)CC4C6)cc3)cc21 ; CC1(C)c2cc(-c3ccccc3)ccc2-c2cc(-c3ccccc3)c(N(c3ccc(C4CCCCC4)cc3)c3ccc(C4C5CC6CC(C5)CC4C6)cc3)cc21]

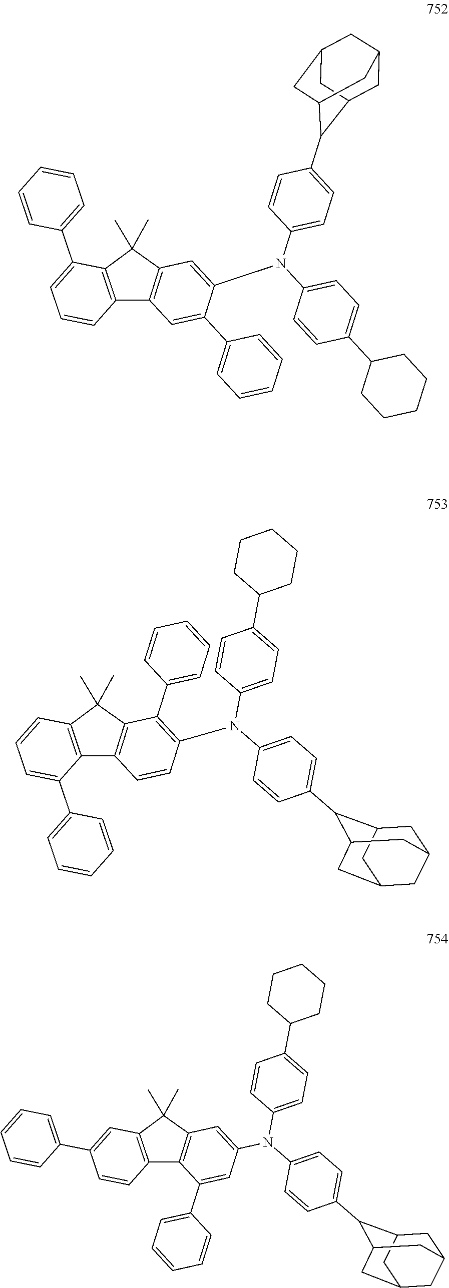 [SMILES: CC1(C)c2cc(N(c3ccc(C4CCCCC4)cc3)c3ccc(C4C5CC6CC(C5)CC4C6)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4C5CC6CC(C5)CC4C6)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4C5CC6CC(C5)CC4C6)cc3)cc21]

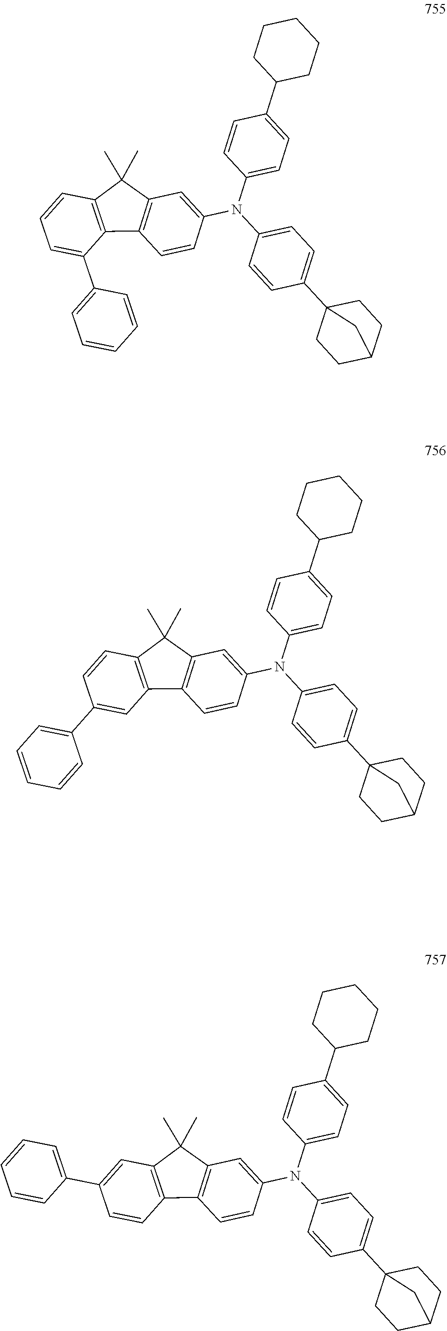 [SMILES: CC1(C)c2cc(N(c3ccc(C4CCCCC4)cc3)c3ccc(C45CCC(CC4)C5)cc3)ccc2-c2c(-c3ccccc3)cccc21 ; CC1(C)c2ccc(-c3ccccc3)cc2-c2ccc(N(c3ccc(C4CCCCC4)cc3)c3ccc(C45CCC(CC4)C5)cc3)cc21 ; CC1(C)c2cc(-c3ccccc3)ccc2-c2ccc(N(c3ccc(C4CCCCC4)cc3)c3ccc(C45CCC(CC4)C5)cc3)cc21]

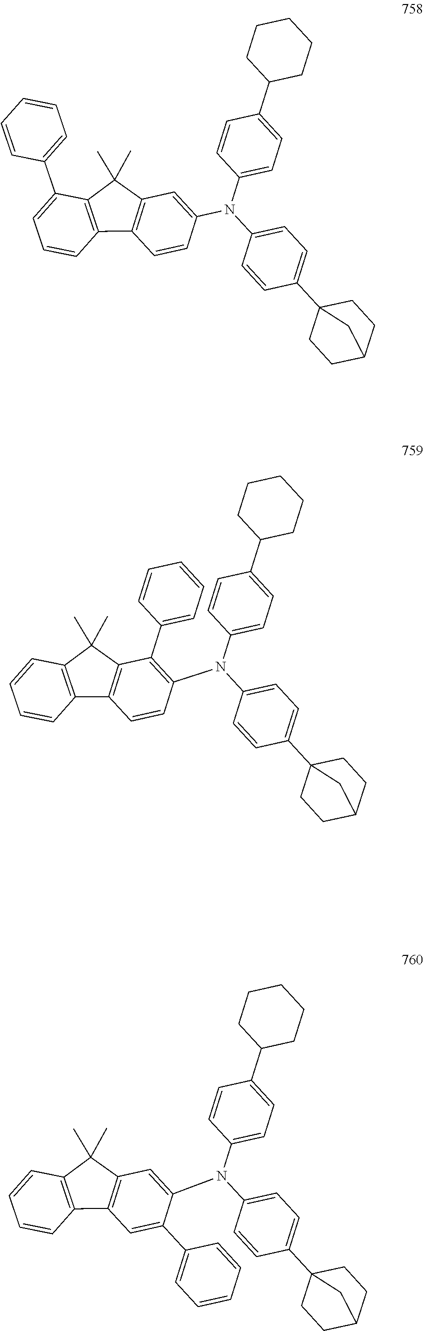 [SMILES: CC1(C)c2cc(N(c3ccc(C4CCCCC4)cc3)c3ccc(C45CCC(CC4)C5)cc3)ccc2-c2cccc(-c3ccccc3)c21 ; CC1(C)c2ccccc2-c2ccc(N(c3ccc(C4CCCCC4)cc3)c3ccc(C45CCC(CC4)C5)cc3)c(-c3ccccc3)c21 ; CC1(C)c2ccccc2-c2cc(-c3ccccc3)c(N(c3ccc(C4CCCCC4)cc3)c3ccc(C45CCC(CC4)C5)cc3)cc21]

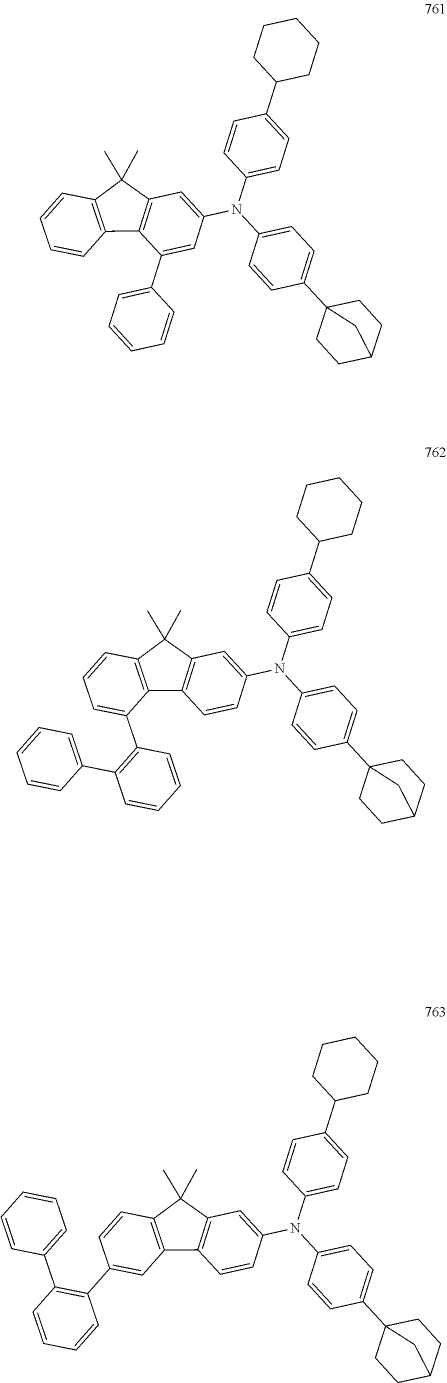 [SMILES: CC1(C)c2ccccc2-c2c(-c3ccccc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C45CCC(CC4)C5)cc3)cc21]

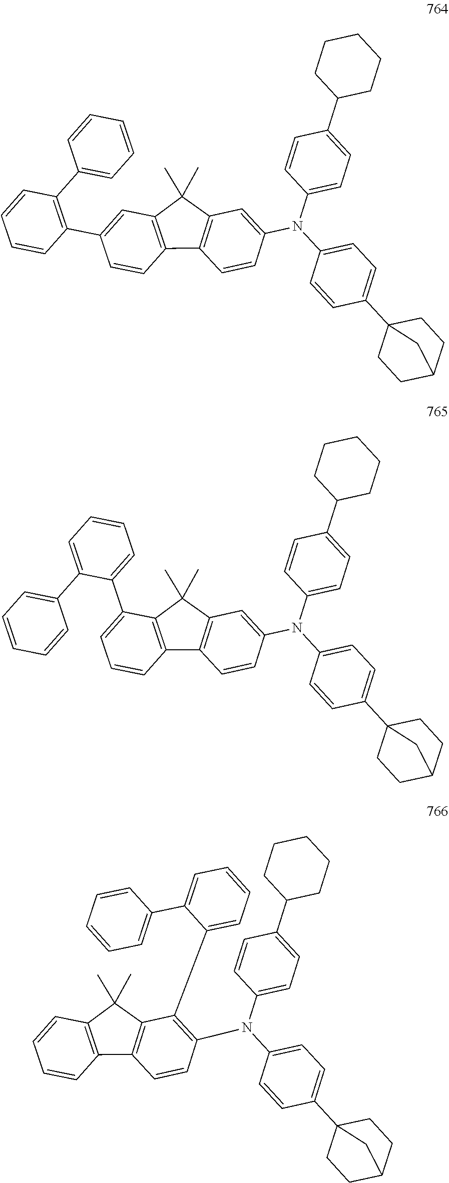 [SMILES: CC1(C)c2cc(-c3ccccc3-c3ccccc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccccc3-c3ccccc3)c21 ; CC1(C)c2ccccc2-c2ccc(N(c3ccc(C4CCCCC4)cc3)c3ccc(C45CCC(CC4)C5)cc3)c(-c3ccccc3-c3ccccc3)c21]

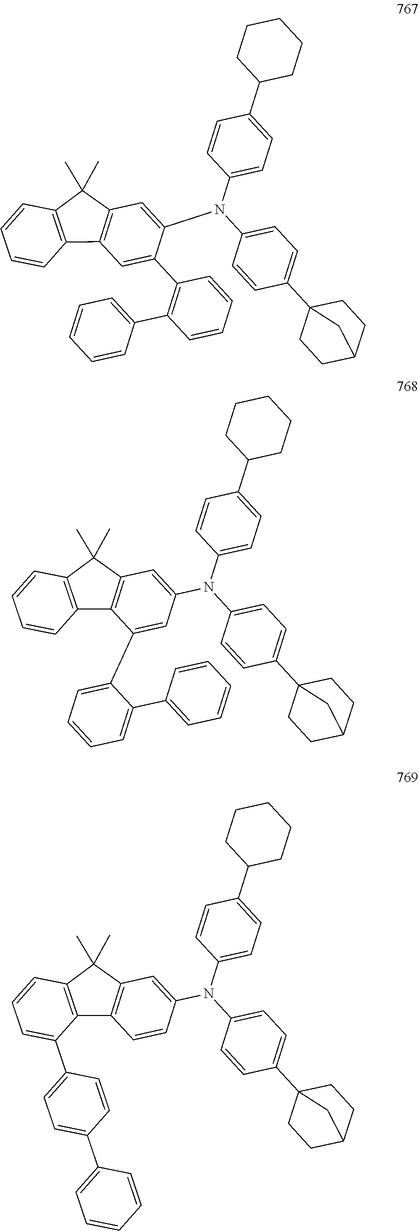 [SMILES: CC1(C)c2ccccc2-c2cc(-c3ccccc3-c3ccccc3)c(N(c3ccc(C4CCCCC4)cc3)c3ccc(C45CCC(CC4)C5)cc3)cc21 ; CC1(C)c2ccccc2-c2c(-c3ccccc3-c3ccccc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc(-c4ccccc4)cc3)cccc21]

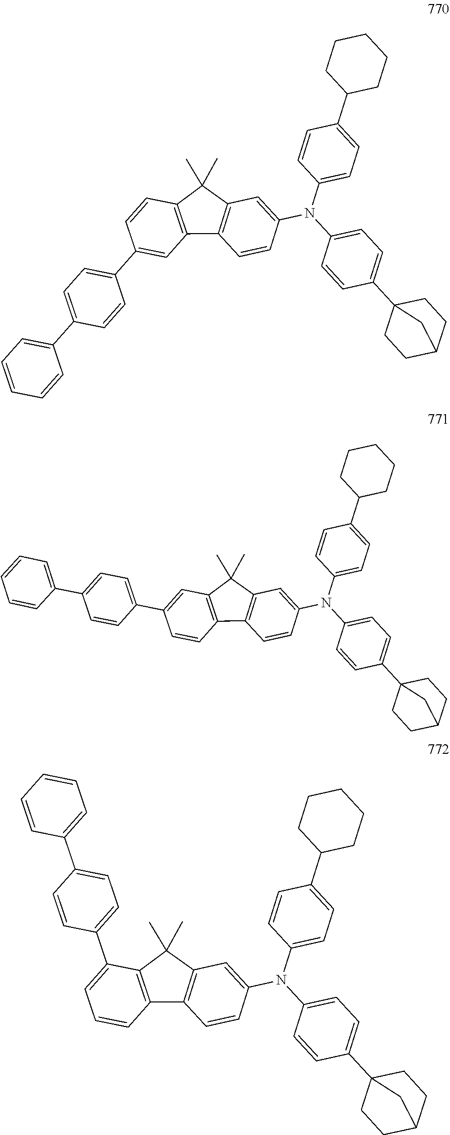 [SMILES: CC1(C)c2ccc(-c3ccc(-c4ccccc4)cc3)cc2-c2ccc(N(c3ccc(C4CCCCC4)cc3)c3ccc(C45CCC(CC4)C5)cc3)cc21 ; CC1(C)c2cc(-c3ccc(-c4ccccc4)cc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc(-c4ccccc4)cc3)c21]

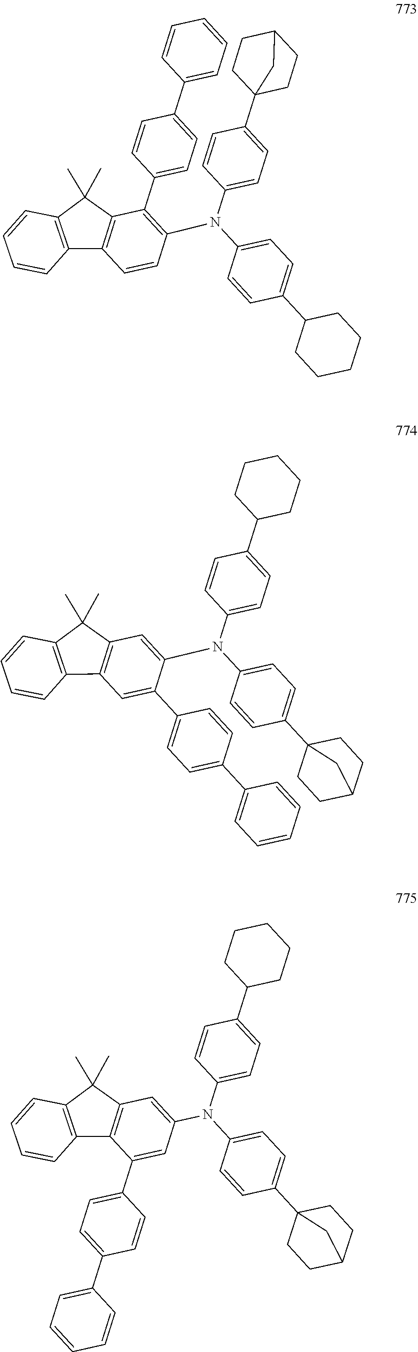 [SMILES: CC1(C)c2ccccc2-c2ccc(N(c3ccc(C4CCCCC4)cc3)c3ccc(C45CCC(CC4)C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C45CCC(CC4)C5)cc3)cc21 ; CC1(C)c2ccccc2-c2c(-c3ccc(-c4ccccc4)cc3)cc(N(c3ccc(C4CCCCC4)cc3)c3ccc(C45CCC(CC4)C5)cc3)cc21]

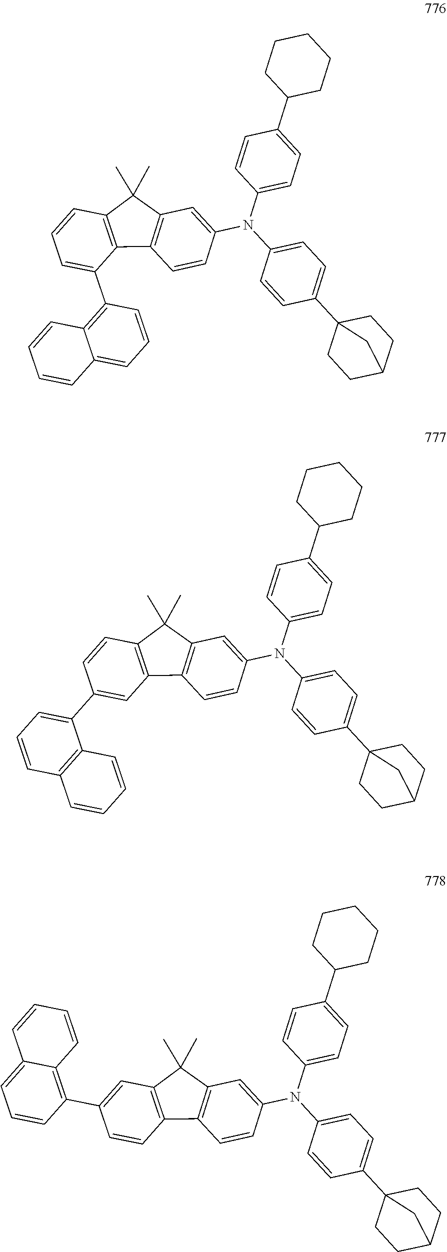 [SMILES: CC1(C)c2cc(N(c3ccc(C4CCCCC4)cc3)c3ccc(C45CCC(CC4)C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C45CCC(CC4)C5)cc3)cc21 ; CC1(C)c2cc(-c3cccc4ccccc34)ccc2-c2ccc(N(c3ccc(C4CCCCC4)cc3)c3ccc(C45CCC(CC4)C5)cc3)cc21]

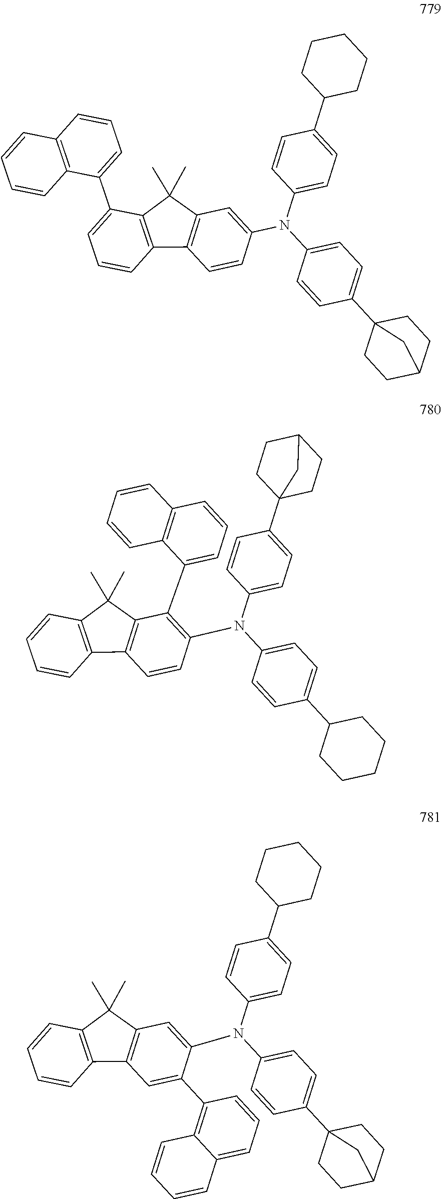 [SMILES: CC1(C)c2cc(N(c3ccc(C4CCCCC4)cc3)c3ccc(C45CCC(CC4)C5)cc3)ccc2-c2cccc(-c3cccc4ccccc34)c21 ; CC1(C)c2ccccc2-c2ccc(N(c3ccc(C4CCCCC4)cc3)c3ccc(C45CCC(CC4)C5)cc3)c(-c3cccc4ccccc34)c21 ; CC1(C)c2ccccc2-c2cc(-c3cccc4ccccc34)c(N(c3ccc(C4CCCCC4)cc3)c3ccc(C45CCC(CC4)C5)cc3)cc21]

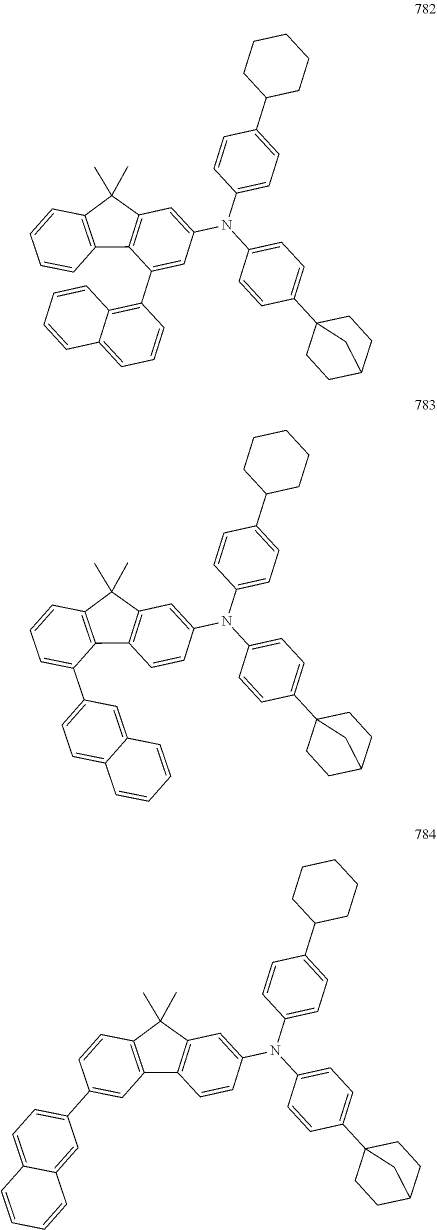 [SMILES: CC1(C)c2ccccc2-c2c(-c3cccc4ccccc34)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C45CCC(CC4)C5)cc3)cc21]

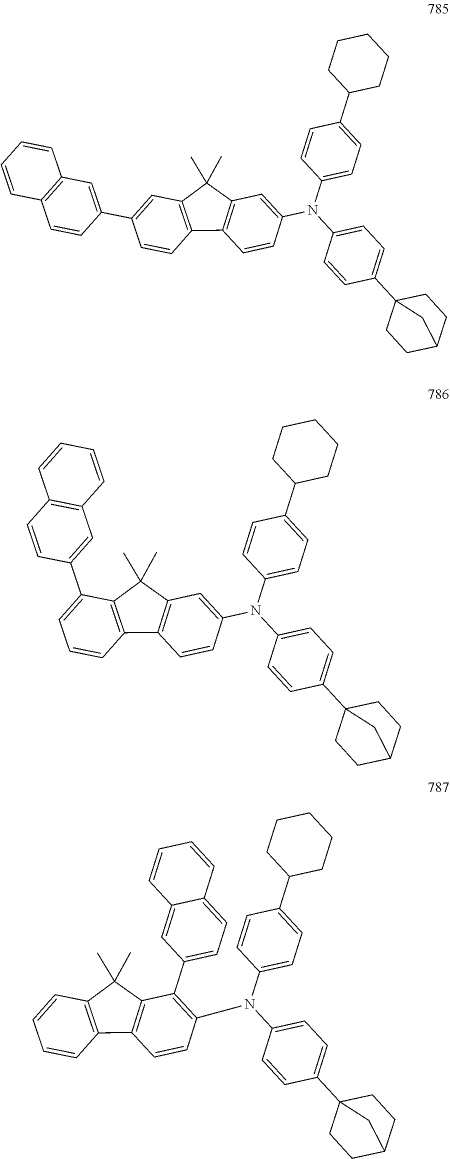 [SMILES: CC1(C)c2cc(-c3ccc4ccccc4c3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc4ccccc4c3)c21 ; CC1(C)c2ccccc2-c2ccc(N(c3ccc(C4CCCCC4)cc3)c3ccc(C45CCC(CC4)C5)cc3)c(-c3ccc4ccccc4c3)c21]

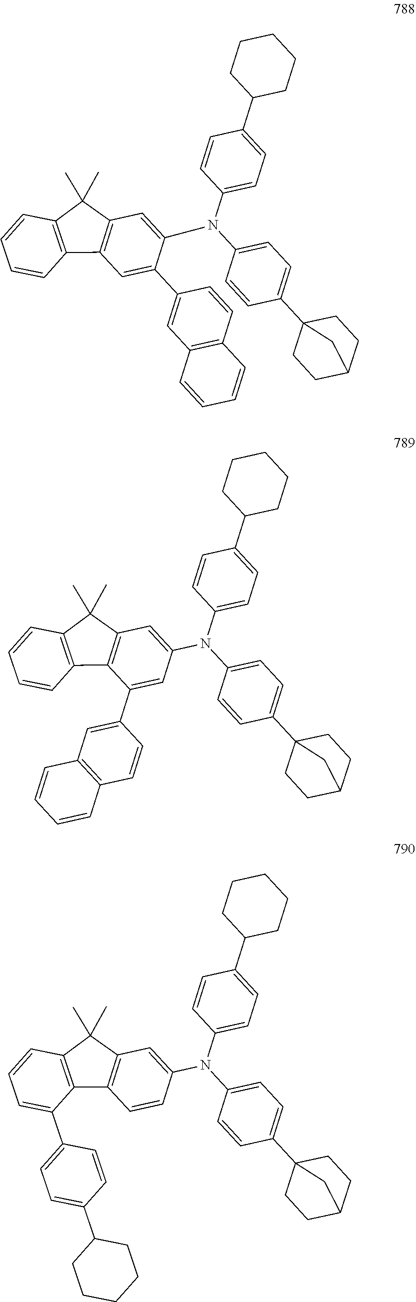 [SMILES: CC1(C)c2ccccc2-c2cc(-c3ccc4ccccc4c3)c(N(c3ccc(C4CCCCC4)cc3)c3ccc(C45CCC(CC4)C5)cc3)cc21 ; CC1(C)c2ccccc2-c2c(-c3ccc4ccccc4c3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc(C4CCCCC4)cc3)cccc21]

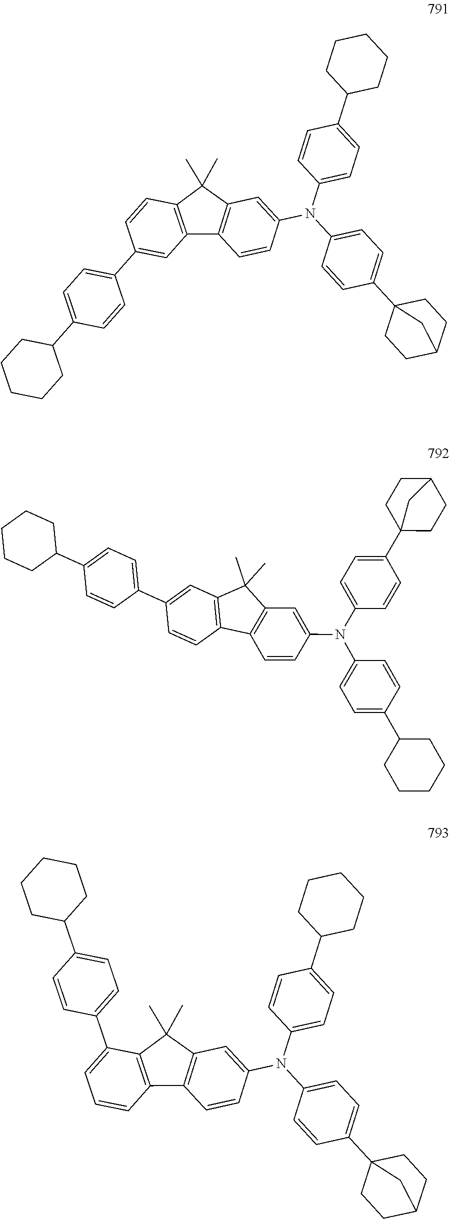 [SMILES: CC1(C)c2ccc(-c3ccc(C4CCCCC4)cc3)cc2-c2ccc(N(c3ccc(C4CCCCC4)cc3)c3ccc(C45CCC(CC4)C5)cc3)cc21 ; CC1(C)c2cc(-c3ccc(C4CCCCC4)cc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc(C4CCCCC4)cc3)c21]

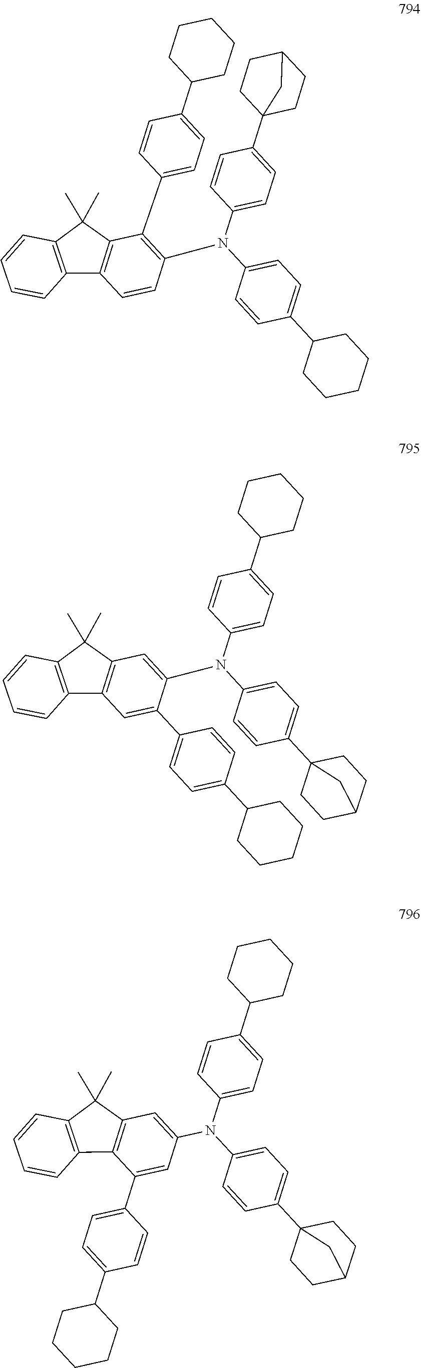 [SMILES: CC1(C)c2ccccc2-c2ccc(N(c3ccc(C4CCCCC4)cc3)c3ccc(C45CCC(CC4)C5)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C45CCC(CC4)C5)cc3)cc21 ; CC1(C)c2ccccc2-c2c(-c3ccc(C4CCCCC4)cc3)cc(N(c3ccc(C4CCCCC4)cc3)c3ccc(C45CCC(CC4)C5)cc3)cc21]

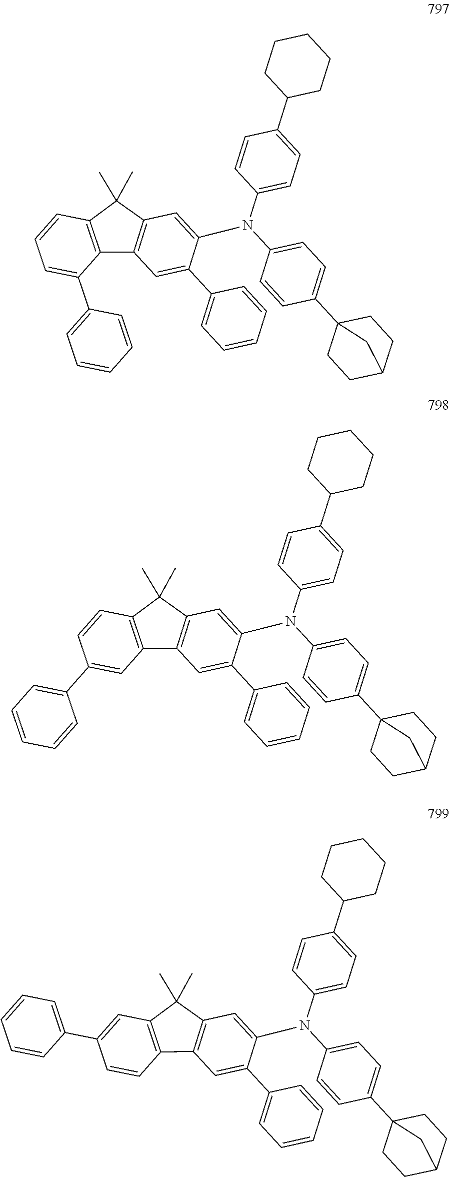 [SMILES: CC1(C)c2cc(N(c3ccc(C4CCCCC4)cc3)c3ccc(C45CCC(CC4)C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C45CCC(CC4)C5)cc3)cc21 ; CC1(C)c2cc(-c3ccccc3)ccc2-c2cc(-c3ccccc3)c(N(c3ccc(C4CCCCC4)cc3)c3ccc(C45CCC(CC4)C5)cc3)cc21]

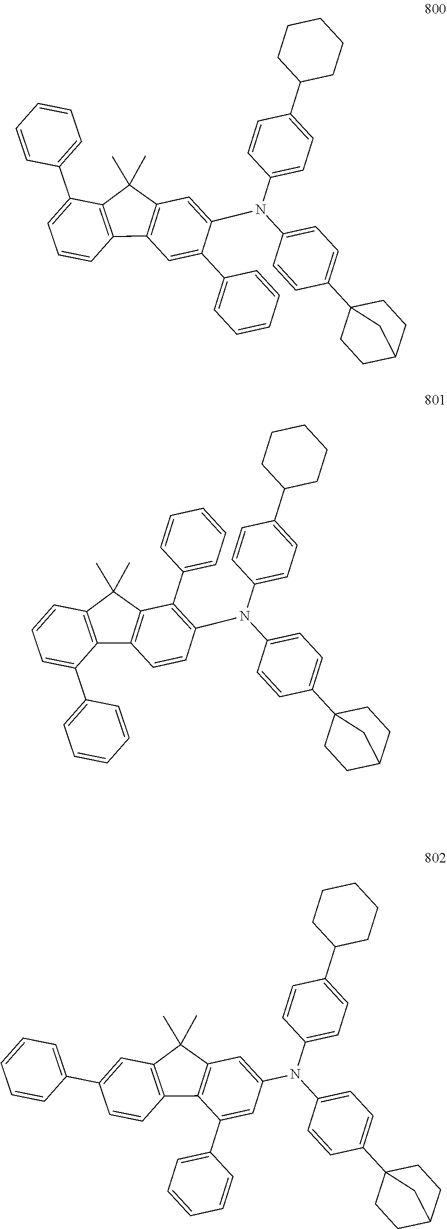 [SMILES: CC1(C)c2cc(N(c3ccc(C4CCCCC4)cc3)c3ccc(C45CCC(CC4)C5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C45CCC(CC4)C5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C45CCC(CC4)C5)cc3)cc21]

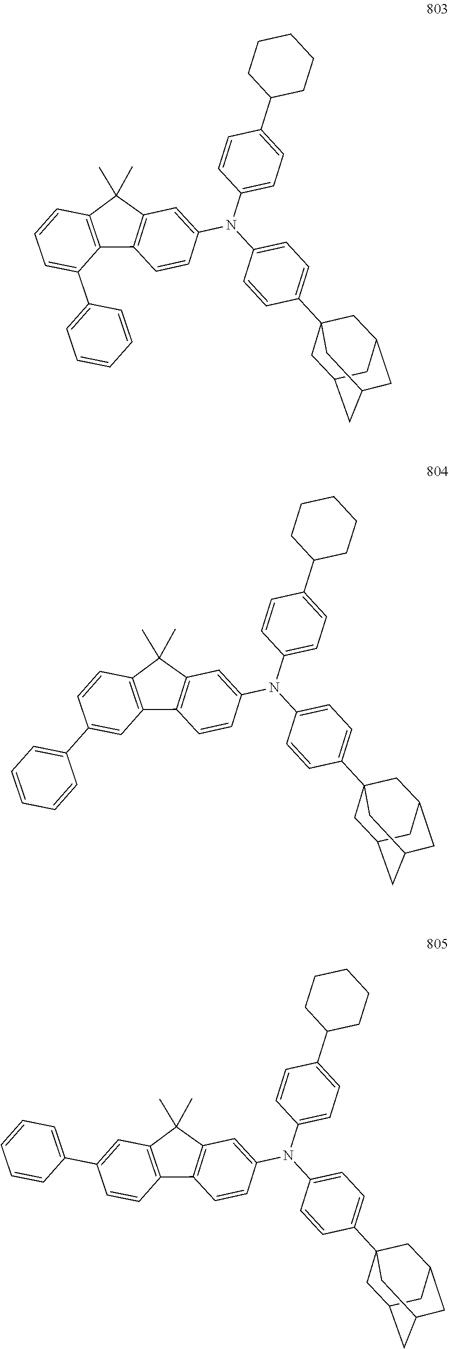 [SMILES: CC1(C)c2cc(N(c3ccc(C4CCCCC4)cc3)c3ccc(C45CC6CC(CC(C6)C4)C5)cc3)ccc2-c2c(-c3ccccc3)cccc21 ; CC1(C)c2ccc(-c3ccccc3)cc2-c2ccc(N(c3ccc(C4CCCCC4)cc3)c3ccc(C45CC6CC(CC(C6)C4)C5)cc3)cc21 ; CC1(C)c2cc(-c3ccccc3)ccc2-c2ccc(N(c3ccc(C4CCCCC4)cc3)c3ccc(C45CC6CC(CC(C6)C4)C5)cc3)cc21]

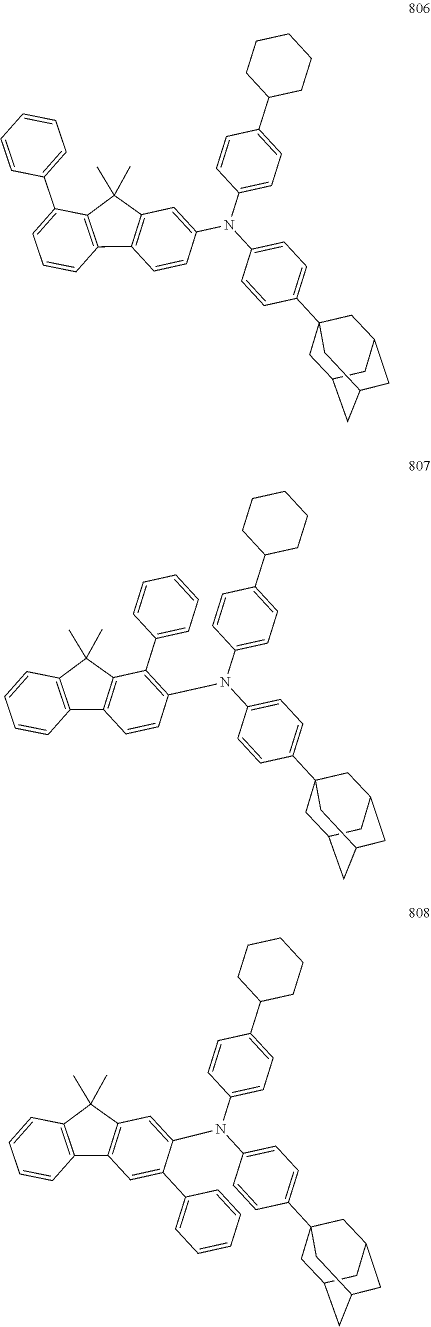 [SMILES: CC1(C)c2cc(N(c3ccc(C4CCCCC4)cc3)c3ccc(C45CC6CC(CC(C6)C4)C5)cc3)ccc2-c2cccc(-c3ccccc3)c21 ; CC1(C)c2ccccc2-c2ccc(N(c3ccc(C4CCCCC4)cc3)c3ccc(C45CC6CC(CC(C6)C4)C5)cc3)c(-c3ccccc3)c21 ; CC1(C)c2ccccc2-c2cc(-c3ccccc3)c(N(c3ccc(C4CCCCC4)cc3)c3ccc(C45CC6CC(CC(C6)C4)C5)cc3)cc21]

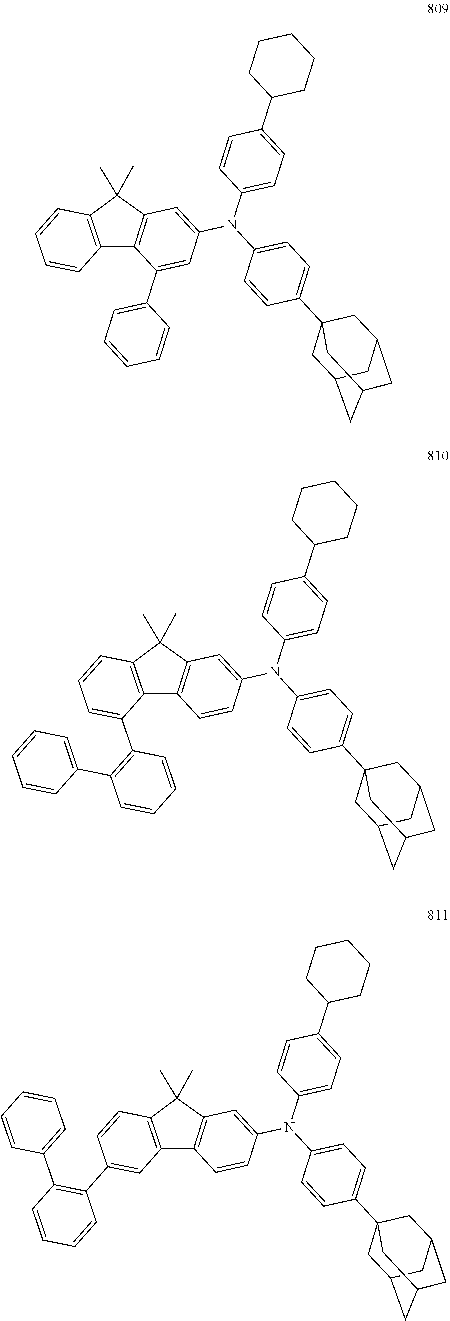 [SMILES: CC1(C)c2ccccc2-c2c(-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C45CC6CC(CC(C6)C4)C5)cc3)cc21]

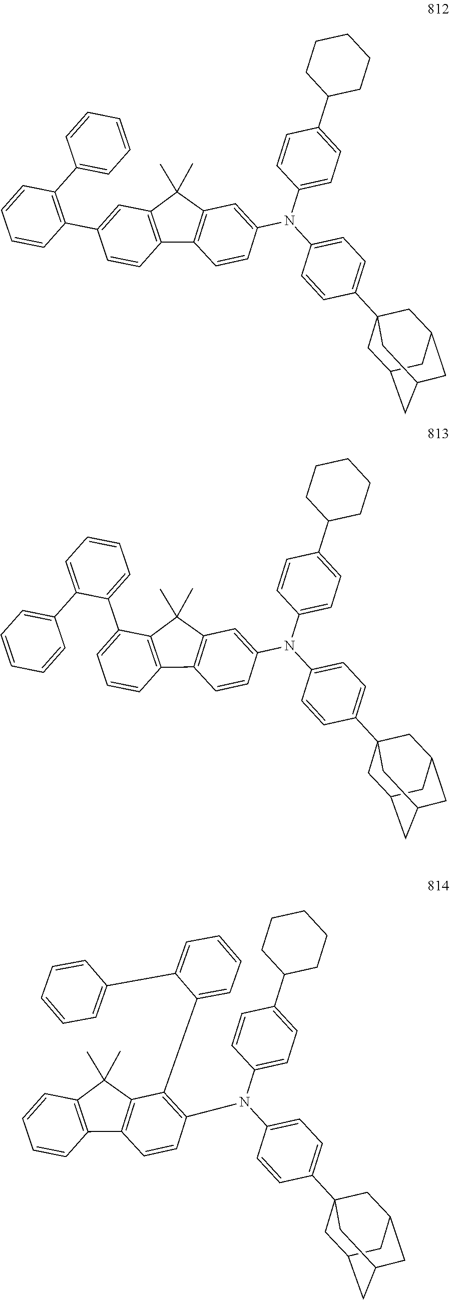 [SMILES: CC1(C)c2cc(-c3ccccc3-c3ccccc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccccc3-c3ccccc3)c21 ; CC1(C)c2ccccc2-c2ccc(N(c3ccc(C4CCCCC4)cc3)c3ccc(C45CC6CC(CC(C6)C4)C5)cc3)c(-c3ccccc3-c3ccccc3)c21]

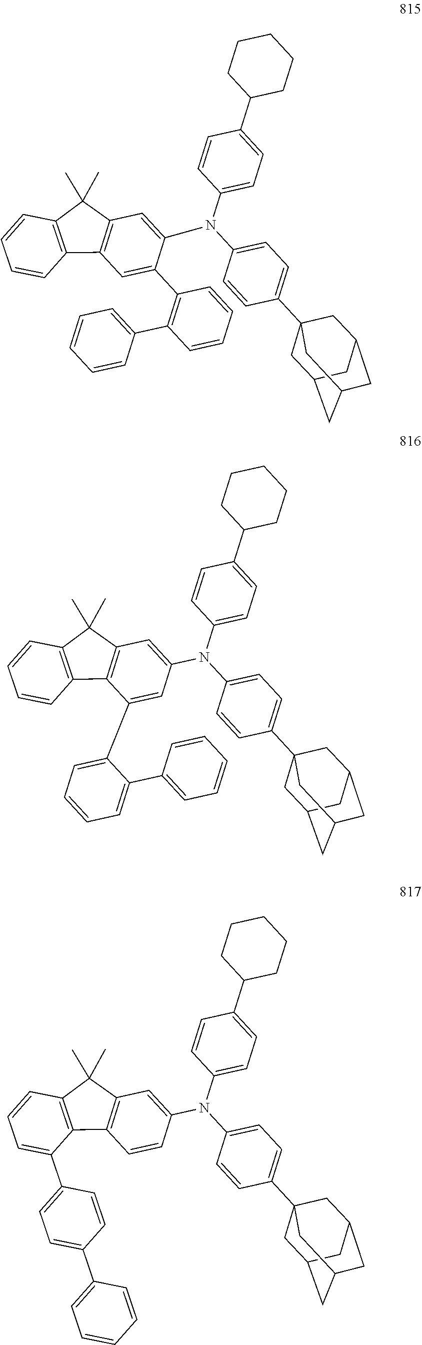 [SMILES: CC1(C)c2ccccc2-c2cc(-c3ccccc3-c3ccccc3)c(N(c3ccc(C4CCCCC4)cc3)c3ccc(C45CC6CC(CC(C6)C4)C5)cc3)cc21 ; CC1(C)c2ccccc2-c2c(-c3ccccc3-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc(-c4ccccc4)cc3)cccc21]

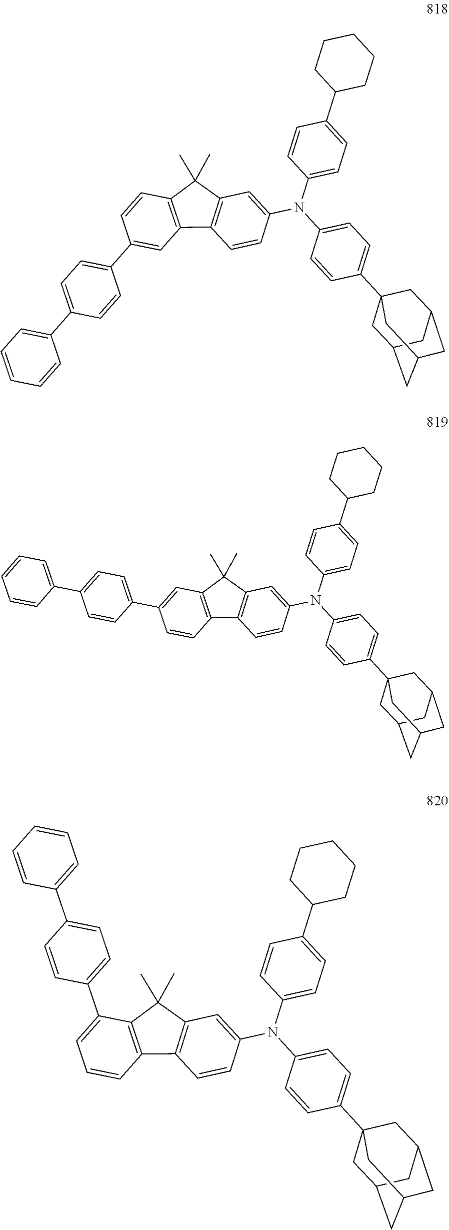 [SMILES: CC1(C)c2ccc(-c3ccc(-c4ccccc4)cc3)cc2-c2ccc(N(c3ccc(C4CCCCC4)cc3)c3ccc(C45CC6CC(CC(C6)C4)C5)cc3)cc21 ; CC1(C)c2cc(-c3ccc(-c4ccccc4)cc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc(-c4ccccc4)cc3)c21]

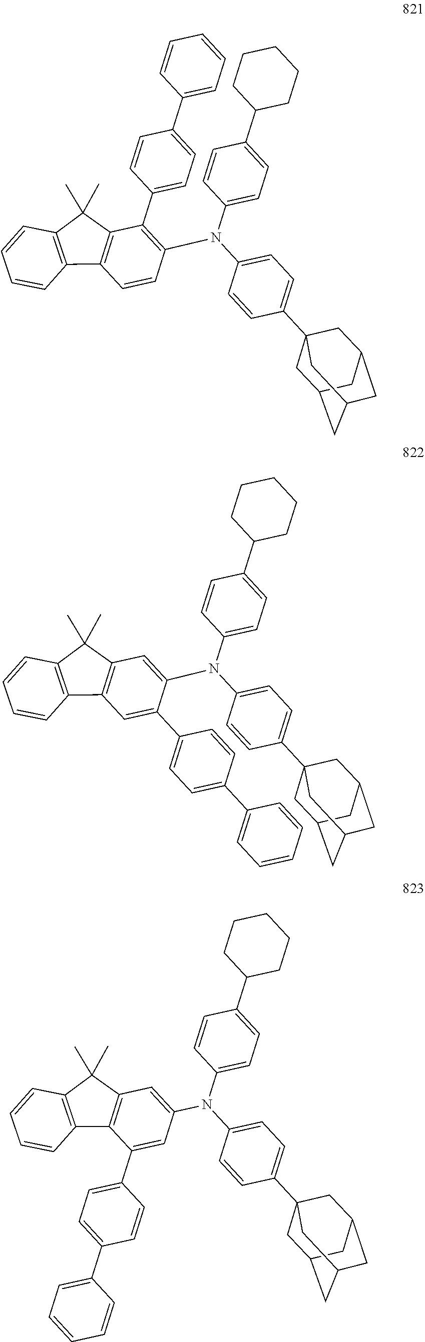 [SMILES: CC1(C)c2ccccc2-c2ccc(N(c3ccc(C4CCCCC4)cc3)c3ccc(C45CC6CC(CC(C6)C4)C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C45CC6CC(CC(C6)C4)C5)cc3)cc21 ; CC1(C)c2ccccc2-c2c(-c3ccc(-c4ccccc4)cc3)cc(N(c3ccc(C4CCCCC4)cc3)c3ccc(C45CC6CC(CC(C6)C4)C5)cc3)cc21]

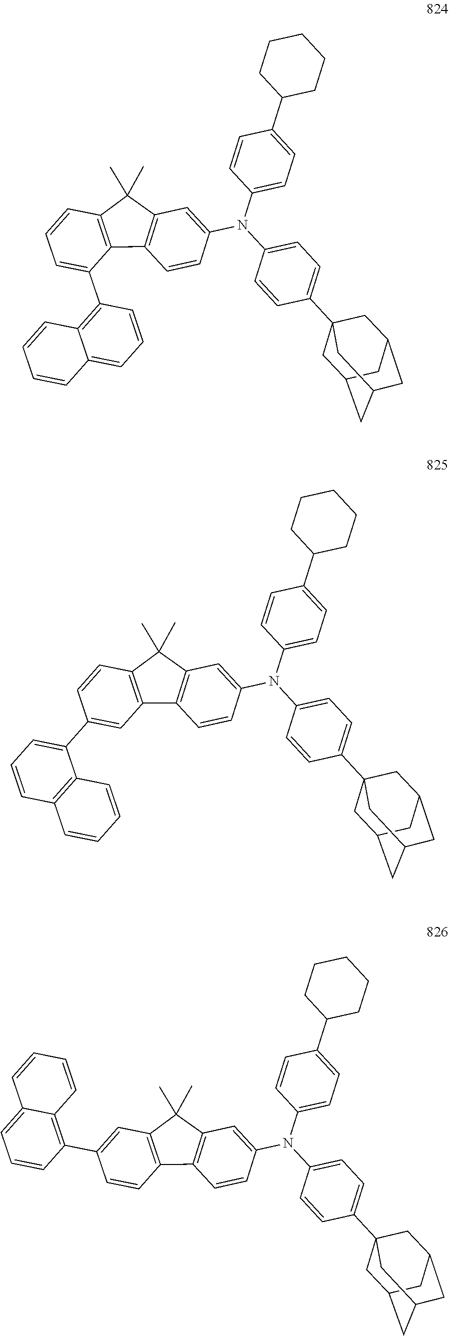 [SMILES: CC1(C)c2cc(N(c3ccc(C4CCCCC4)cc3)c3ccc(C45CC6CC(CC(C6)C4)C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C45CC6CC(CC(C6)C4)C5)cc3)cc21 ; CC1(C)c2cc(-c3cccc4ccccc34)ccc2-c2ccc(N(c3ccc(C4CCCCC4)cc3)c3ccc(C45CC6CC(CC(C6)C4)C5)cc3)cc21]

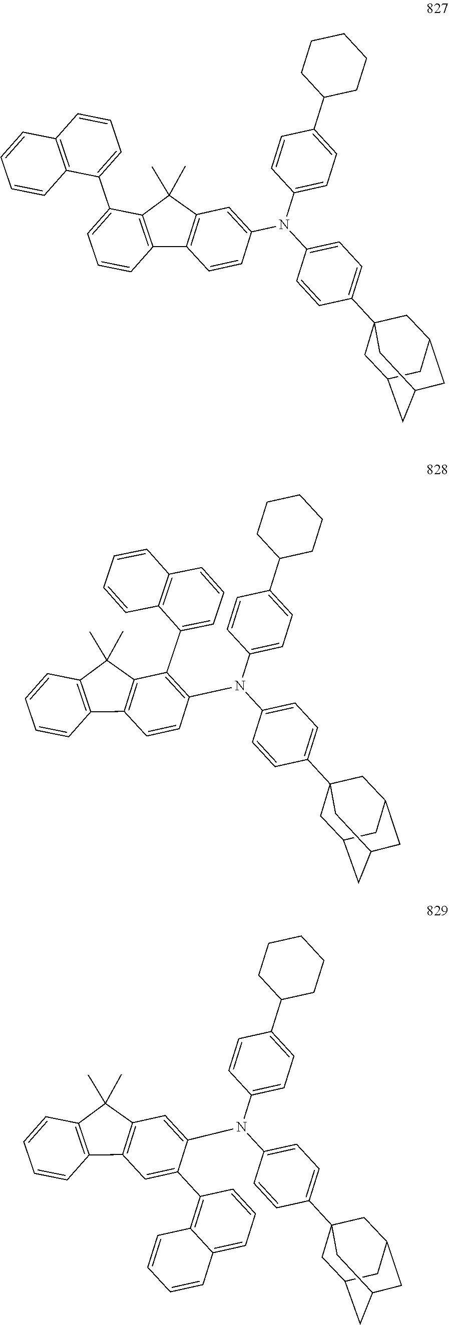 [SMILES: CC1(C)c2cc(N(c3ccc(C4CCCCC4)cc3)c3ccc(C45CC6CC(CC(C6)C4)C5)cc3)ccc2-c2cccc(-c3cccc4ccccc34)c21 ; CC1(C)c2ccccc2-c2ccc(N(c3ccc(C4CCCCC4)cc3)c3ccc(C45CC6CC(CC(C6)C4)C5)cc3)c(-c3cccc4ccccc34)c21 ; CC1(C)c2ccccc2-c2cc(-c3cccc4ccccc34)c(N(c3ccc(C4CCCCC4)cc3)c3ccc(C45CC6CC(CC(C6)C4)C5)cc3)cc21]

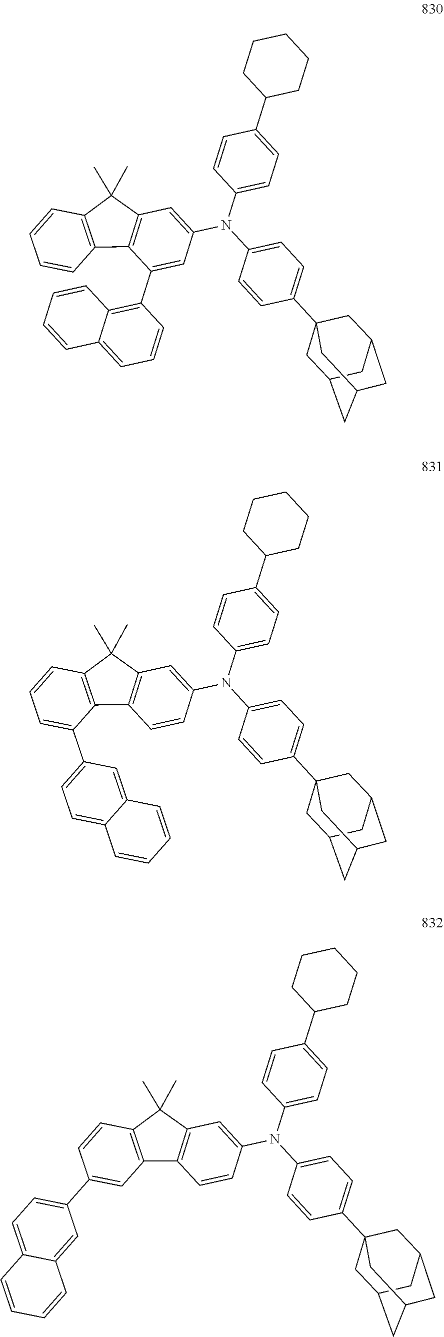 [SMILES: CC1(C)c2ccccc2-c2c(-c3cccc4ccccc34)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C45CC6CC(CC(C6)C4)C5)cc3)cc21]

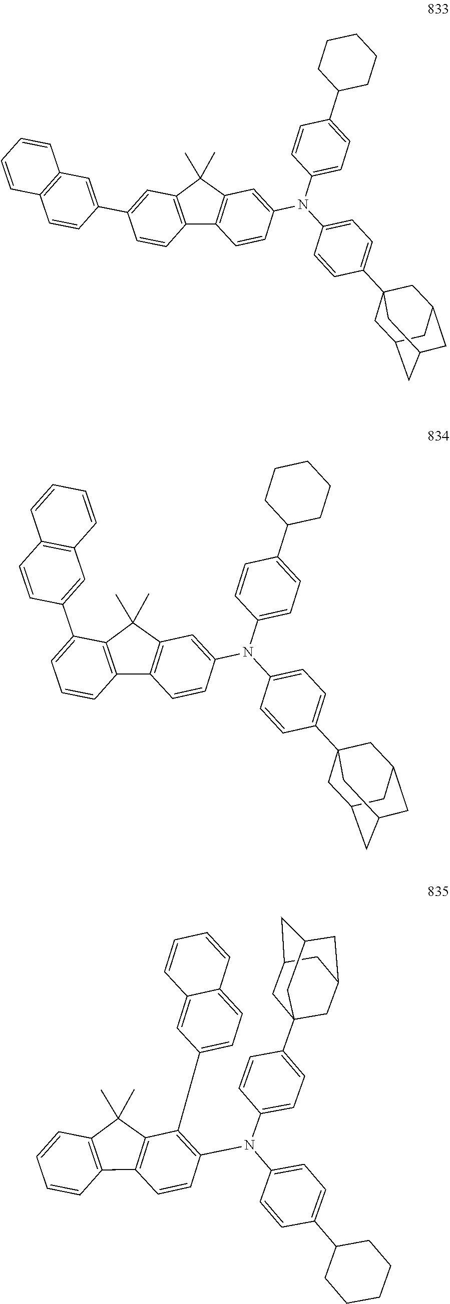 [SMILES: CC1(C)c2cc(-c3ccc4ccccc4c3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc4ccccc4c3)c21 ; CC1(C)c2ccccc2-c2ccc(N(c3ccc(C4CCCCC4)cc3)c3ccc(C45CC6CC(CC(C6)C4)C5)cc3)c(-c3ccc4ccccc4c3)c21]

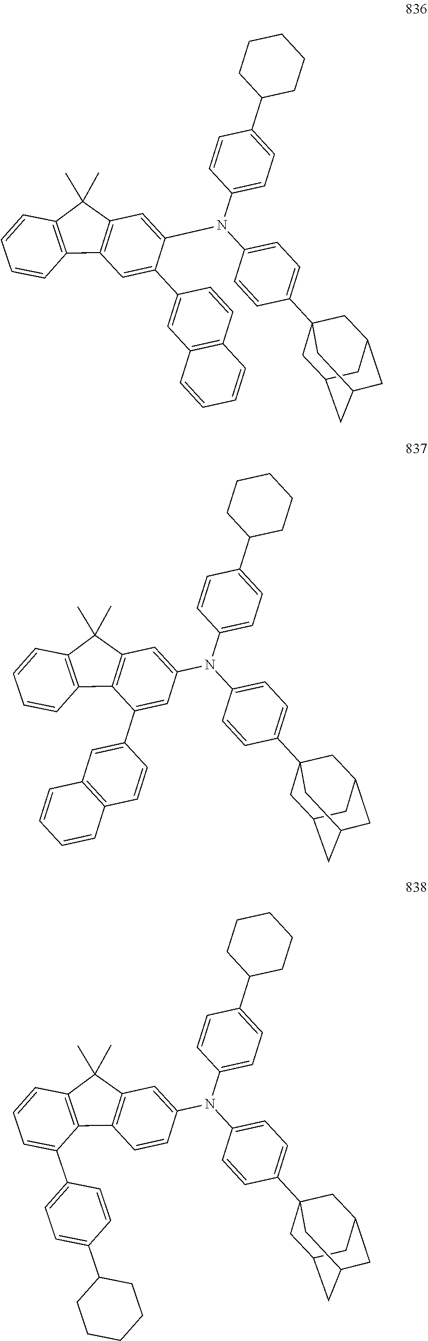 [SMILES: CC1(C)c2ccccc2-c2cc(-c3ccc4ccccc4c3)c(N(c3ccc(C4CCCCC4)cc3)c3ccc(C45CC6CC(CC(C6)C4)C5)cc3)cc21 ; CC1(C)c2ccccc2-c2c(-c3ccc4ccccc4c3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc(C4CCCCC4)cc3)cccc21]

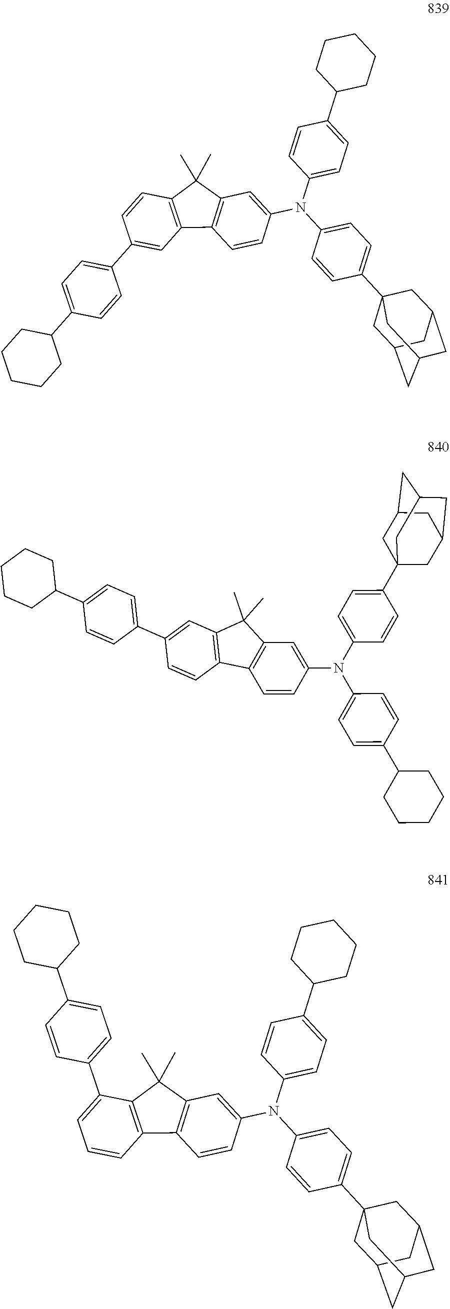 [SMILES: CC1(C)c2ccc(-c3ccc(C4CCCCC4)cc3)cc2-c2ccc(N(c3ccc(C4CCCCC4)cc3)c3ccc(C45CC6CC(CC(C6)C4)C5)cc3)cc21 ; CC1(C)c2cc(-c3ccc(C4CCCCC4)cc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc(C4CCCCC4)cc3)c21]

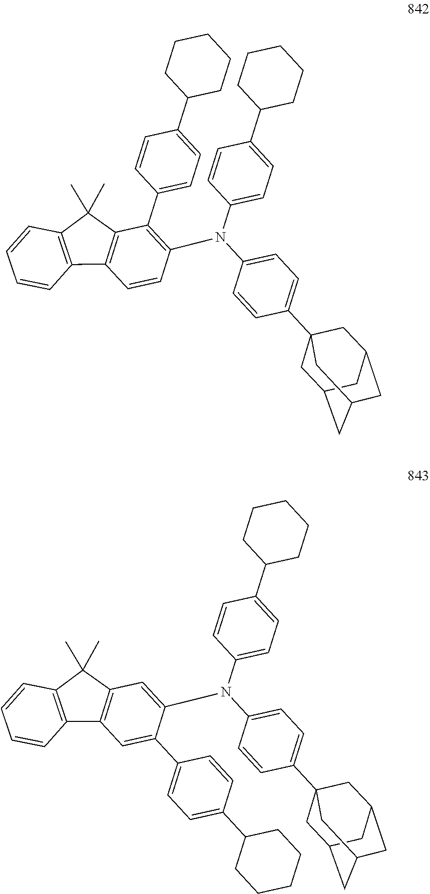 [SMILES: CC1(C)c2ccccc2-c2ccc(N(c3ccc(C4CCCCC4)cc3)c3ccc(C45CC6CC(CC(C6)C4)C5)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C45CC6CC(CC(C6)C4)C5)cc3)cc21]

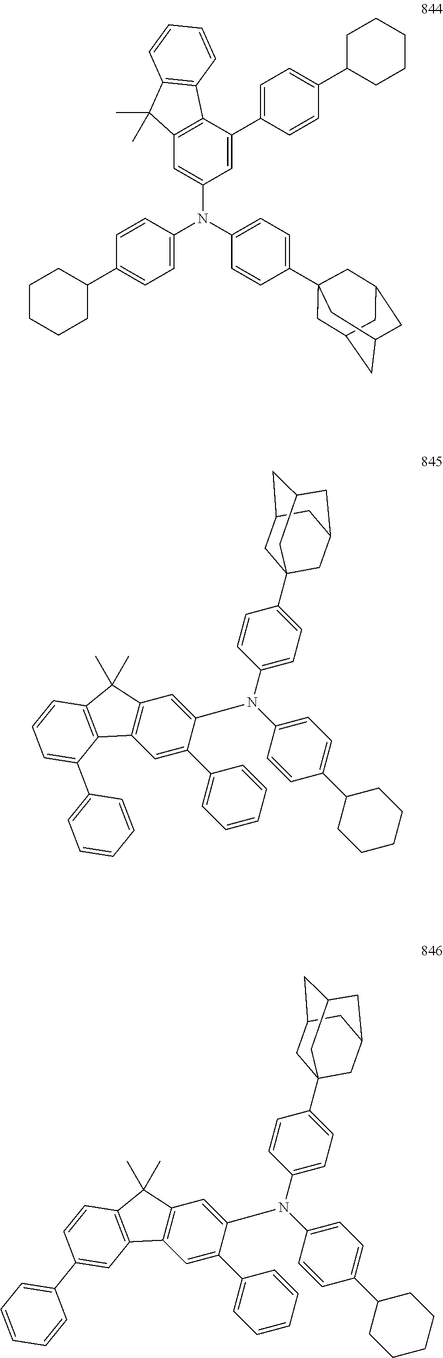 [SMILES: CC1(C)c2ccccc2-c2c(-c3ccc(C4CCCCC4)cc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C45CC6CC(CC(C6)C4)C5)cc3)cc21]

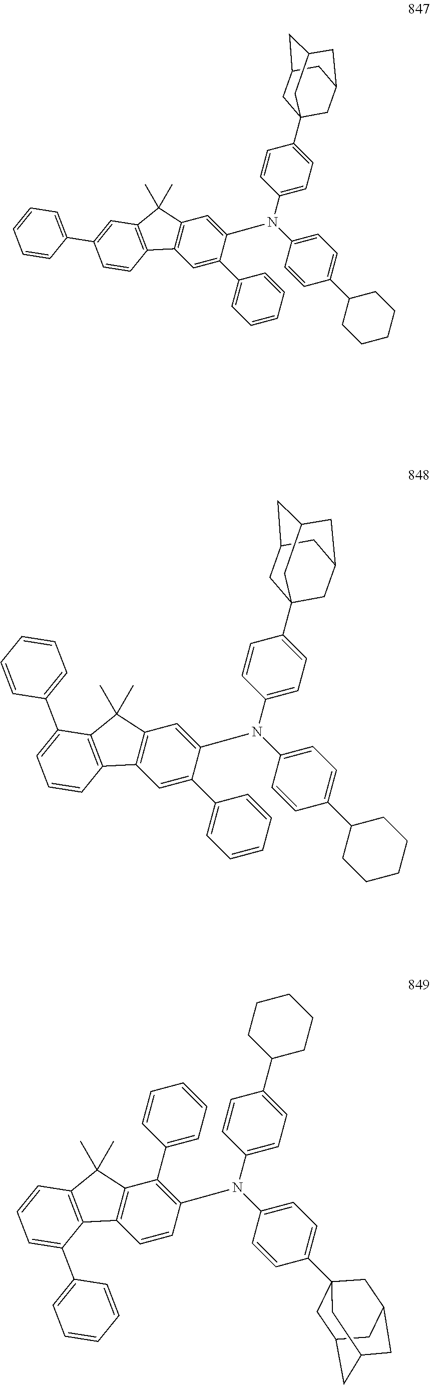 [SMILES: CC1(C)c2cc(-c3ccccc3)ccc2-c2cc(-c3ccccc3)c(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C45CC6CC(CC(C6)C4)C5)cc3)c(-c3ccccc3)c21]

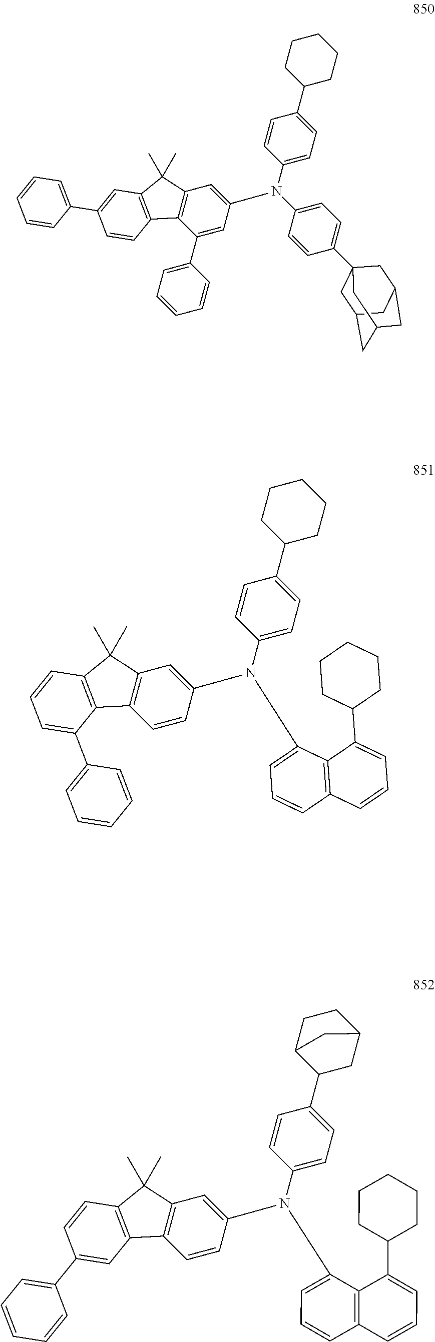 [SMILES: CC1(C)c2cc(-c3ccccc3)ccc2-c2c(-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3cccc4cccc(C5CCCCC5)c34)ccc2-c2c(-c3ccccc3)cccc21 ; CC1(C)c2ccc(-c3ccccc3)cc2-c2ccc(N(c3ccc(C4CC5CCC4C5)cc3)c3cccc4cccc(C5CCCCC5)c34)cc21]

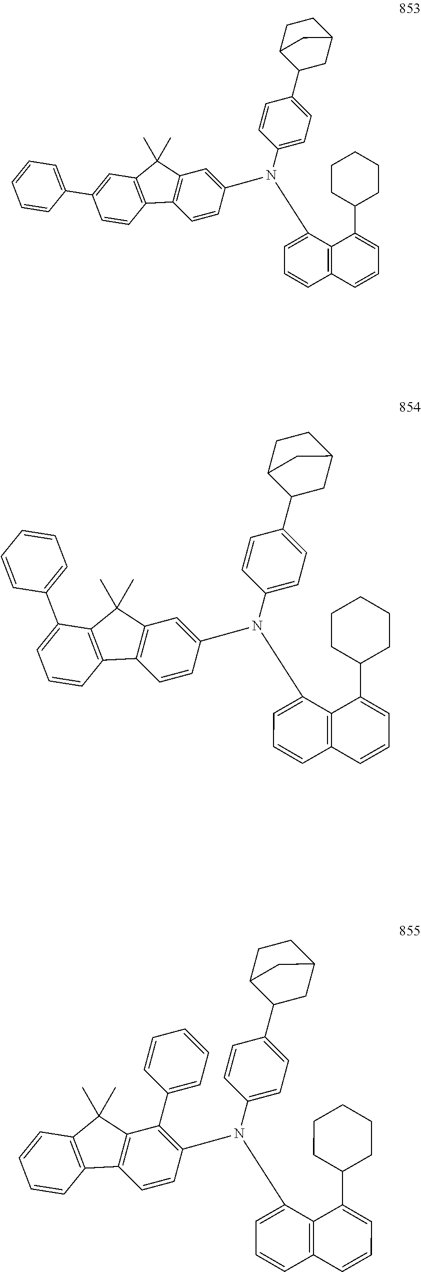 [SMILES: CC1(C)c2cc(-c3ccccc3)ccc2-c2ccc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cccc(C5CCCCC5)c34)ccc2-c2cccc(-c3ccccc3)c21 ; CC1(C)c2ccccc2-c2ccc(N(c3ccc(C4CC5CCC4C5)cc3)c3cccc4cccc(C5CCCCC5)c34)c(-c3ccccc3)c21]

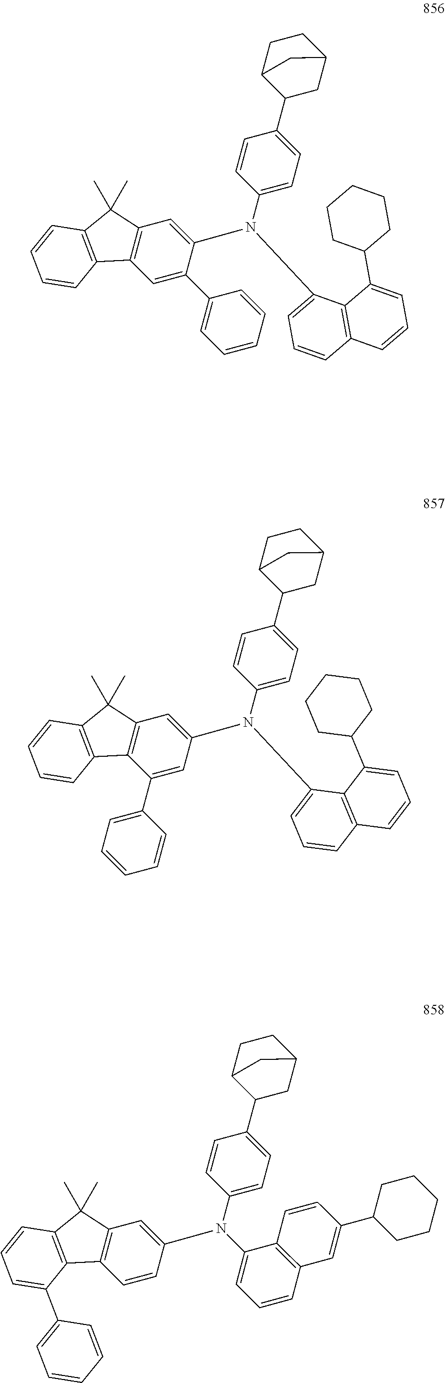 [SMILES: CC1(C)c2ccccc2-c2cc(-c3ccccc3)c(N(c3ccc(C4CC5CCC4C5)cc3)c3cccc4cccc(C5CCCCC5)c34)cc21 ; CC1(C)c2ccccc2-c2c(-c3ccccc3)cc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cc(C5CCCCC5)ccc34)ccc2-c2c(-c3ccccc3)cccc21]

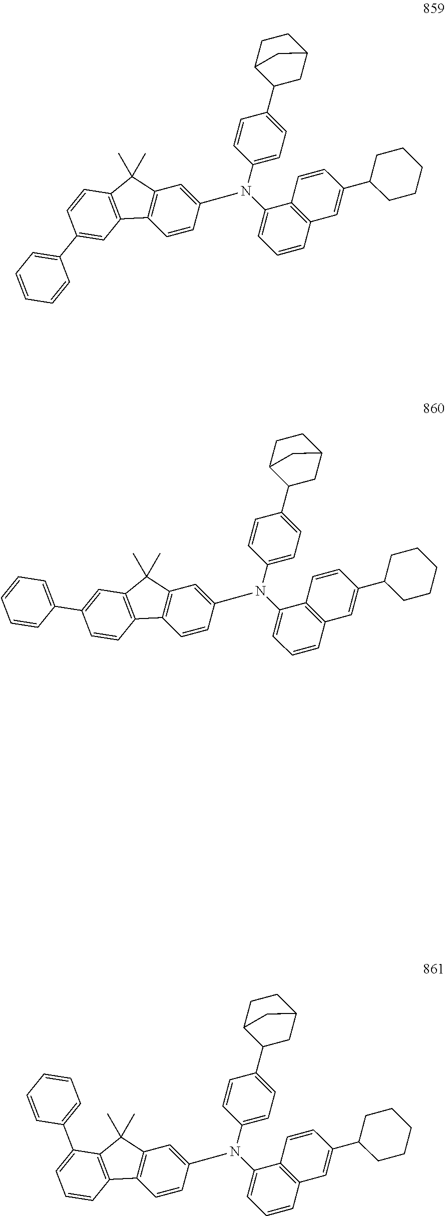 [SMILES: CC1(C)c2ccc(-c3ccccc3)cc2-c2ccc(N(c3ccc(C4CC5CCC4C5)cc3)c3cccc4cc(C5CCCCC5)ccc34)cc21 ; CC1(C)c2cc(-c3ccccc3)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3ccccc3)c21]

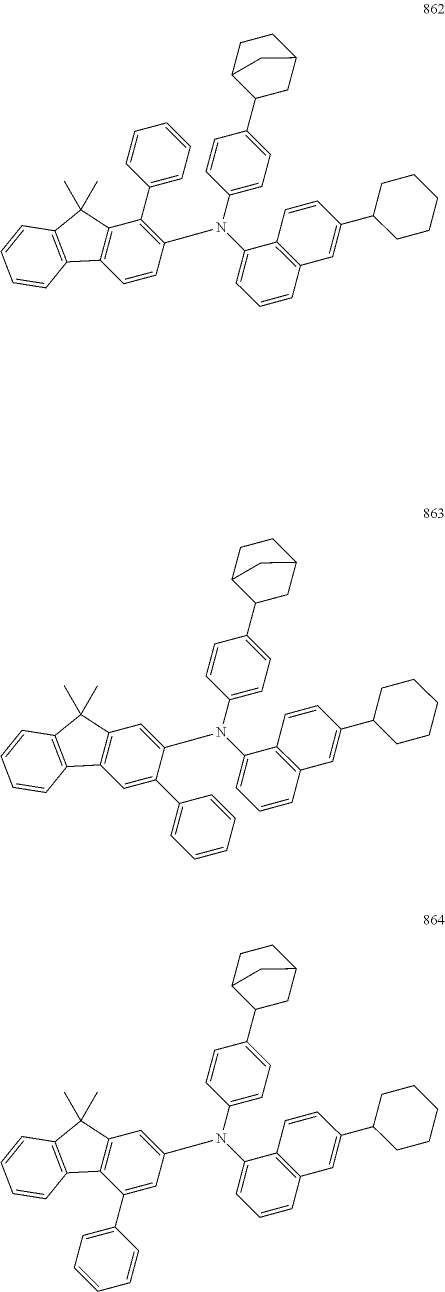 [SMILES: CC1(C)c2ccccc2-c2ccc(N(c3ccc(C4CC5CCC4C5)cc3)c3cccc4cc(C5CCCCC5)ccc34)c(-c3ccccc3)c21 ; CC1(C)c2ccccc2-c2cc(-c3ccccc3)c(N(c3ccc(C4CC5CCC4C5)cc3)c3cccc4cc(C5CCCCC5)ccc34)cc21 ; CC1(C)c2ccccc2-c2c(-c3ccccc3)cc(N(c3ccc(C4CC5CCC4C5)cc3)c3cccc4cc(C5CCCCC5)ccc34)cc21]

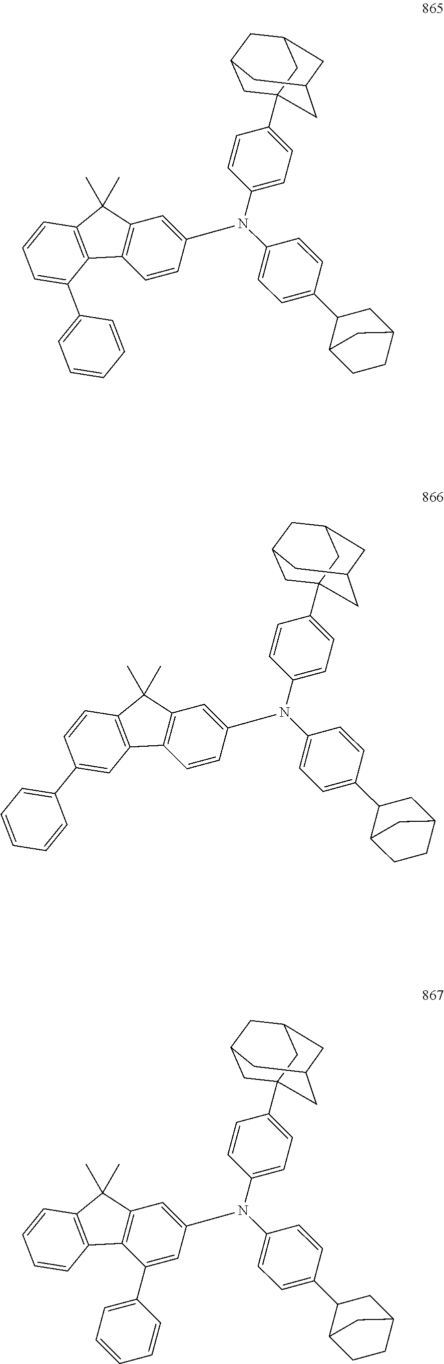 [SMILES: CC1(C)c2cc(N(c3ccc(C4CC5CCC4C5)cc3)c3ccc(C45CC6CC(CC(C6)C4)C5)cc3)ccc2-c2c(-c3ccccc3)cccc21 ; CC1(C)c2ccc(-c3ccccc3)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3ccccc3)cc(N(c3ccc(C4CC5CCC4C5)cc3)c3ccc(C45CC6CC(CC(C6)C4)C5)cc3)cc21]

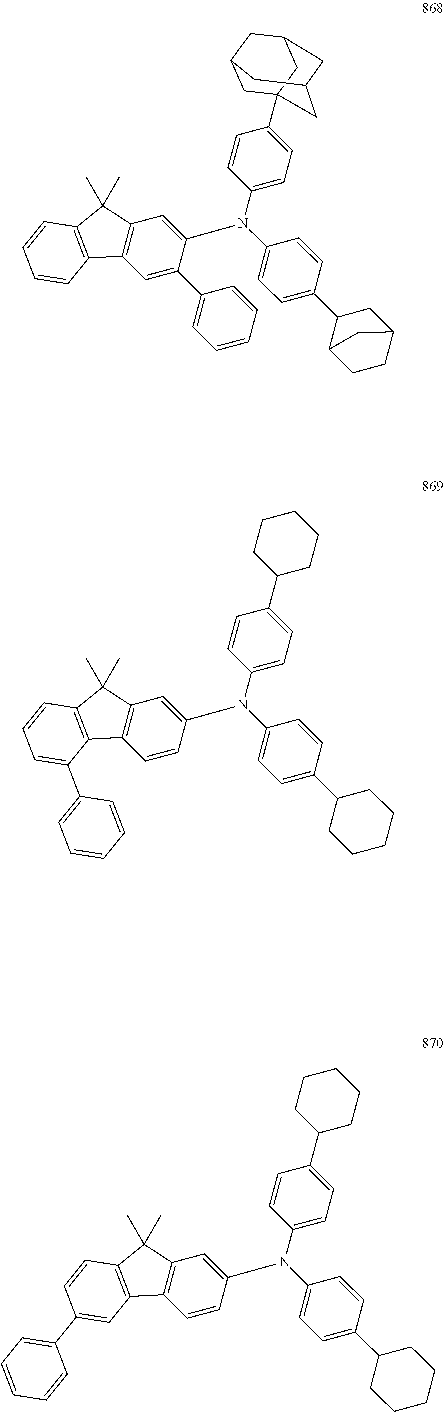 [SMILES: CC1(C)c2ccccc2-c2cc(-c3ccccc3)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C4CCCCC4)cc3)ccc2-c2c(-c3ccccc3)cccc21 ; CC1(C)c2ccc(-c3ccccc3)cc2-c2ccc(N(c3ccc(C4CCCCC4)cc3)c3ccc(C4CCCCC4)cc3)cc21]

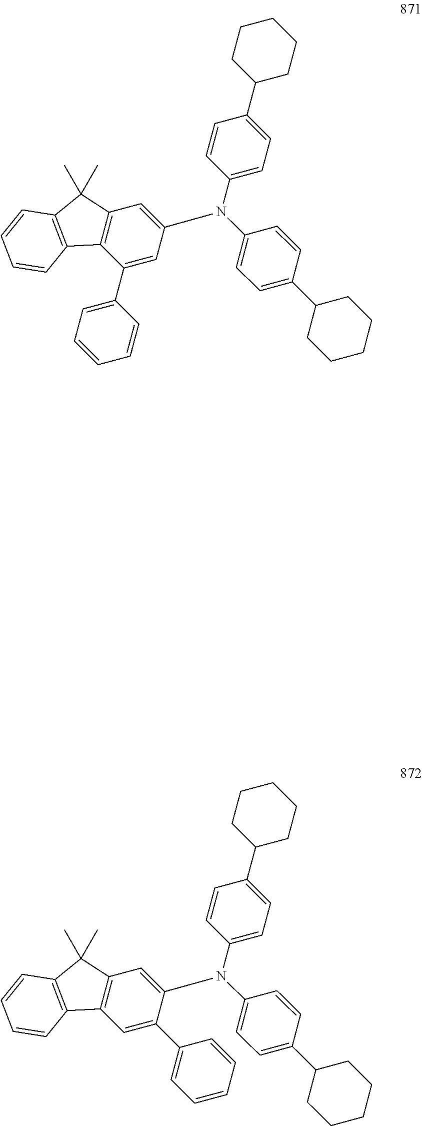 [SMILES: CC1(C)c2ccccc2-c2c(-c3ccccc3)cc(N(c3ccc(C4CCCCC4)cc3)c3ccc(C4CCCCC4)cc3)cc21 ; CC1(C)c2ccccc2-c2cc(-c3ccccc3)c(N(c3ccc(C4CCCCC4)cc3)c3ccc(C4CCCCC4)cc3)cc21]

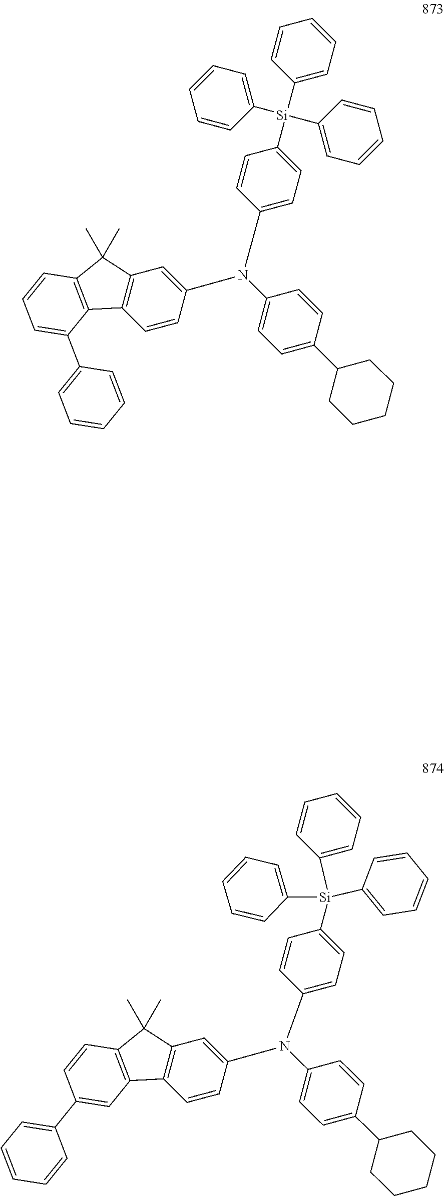 [SMILES: CC1(C)c2cc(N(c3ccc(C4CCCCC4)cc3)c3ccc([Si](c4ccccc4)(c4ccccc4)c4ccccc4)cc3)ccc2-c2c(-c3ccccc3)cccc21 ; CC1(C)c2ccc(-c3ccccc3)cc2-c2ccc(N(c3ccc(C4CCCCC4)cc3)c3ccc([Si](c4ccccc4)(c4ccccc4)c4ccccc4)cc3)cc21]

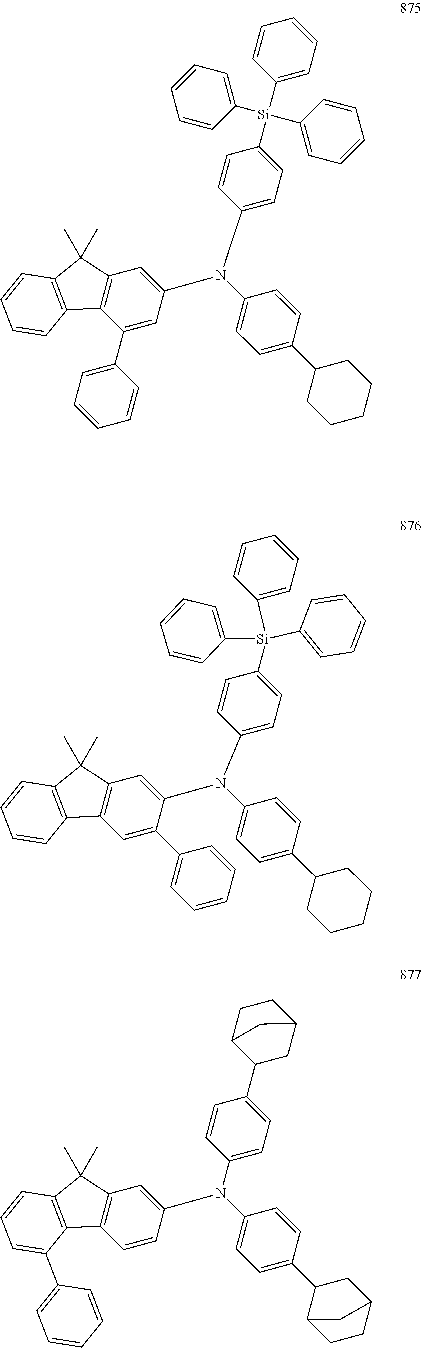 [SMILES: CC1(C)c2ccccc2-c2c(-c3ccccc3)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3ccccc3)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3ccccc3)cccc21]

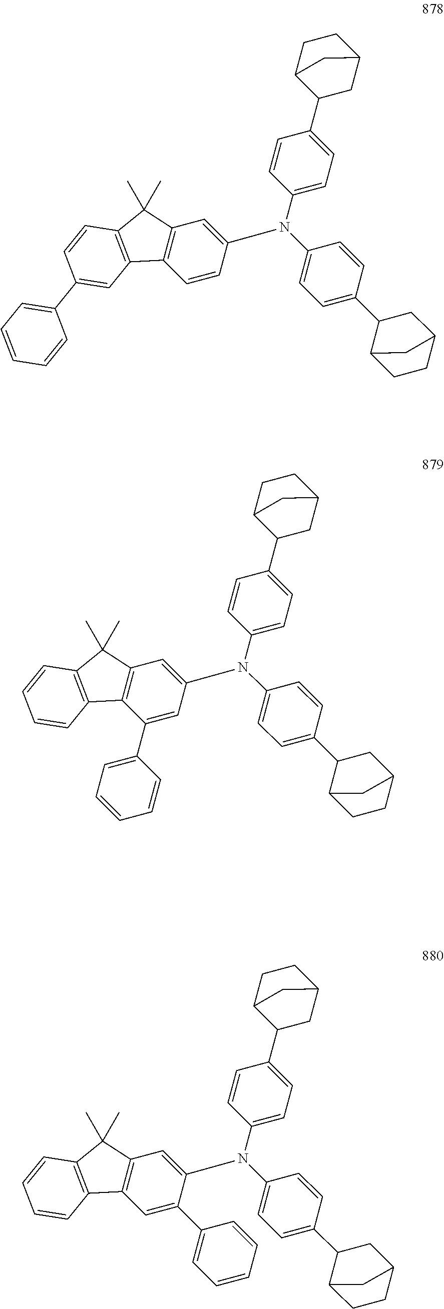 [SMILES: CC1(C)c2ccc(-c3ccccc3)cc2-c2ccc(N(c3ccc(C4CC5CCC4C5)cc3)c3ccc(C4CC5CCC4C5)cc3)cc21 ; CC1(C)c2ccccc2-c2c(-c3ccccc3)cc(N(c3ccc(C4CC5CCC4C5)cc3)c3ccc(C4CC5CCC4C5)cc3)cc21 ; CC1(C)c2ccccc2-c2cc(-c3ccccc3)c(N(c3ccc(C4CC5CCC4C5)cc3)c3ccc(C4CC5CCC4C5)cc3)cc21]

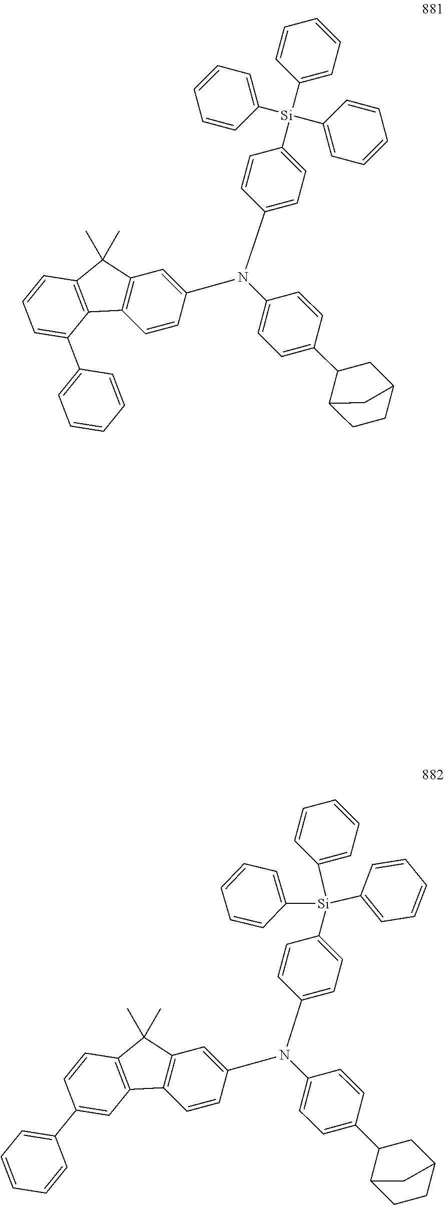 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3ccccc3)cccc21 ; CC1(C)c2ccc(-c3ccccc3)cc2-c2ccc(N(c3ccc(C4CC5CCC4C5)cc3)c3ccc([Si](c4ccccc4)(c4ccccc4)c4ccccc4)cc3)cc21]

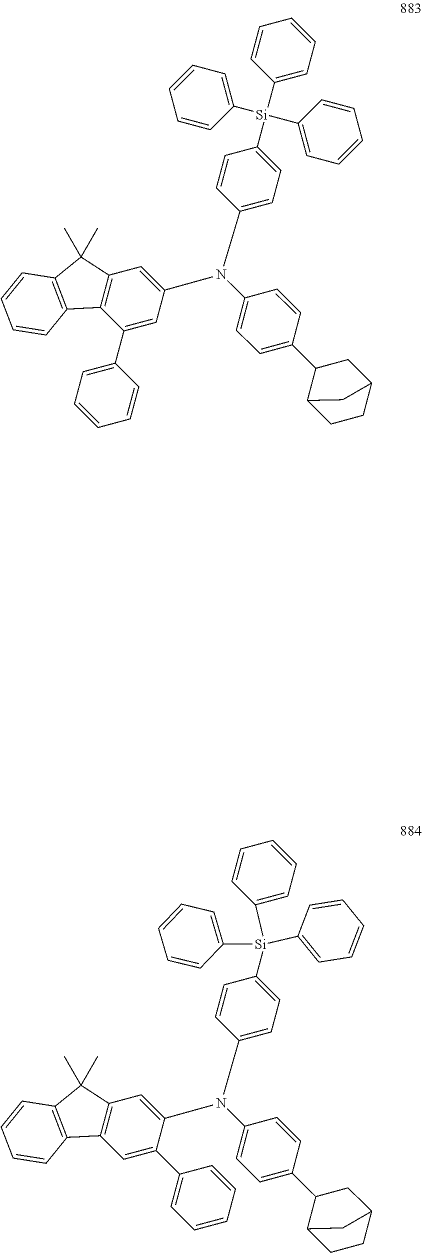 [SMILES: CC1(C)c2ccccc2-c2c(-c3ccccc3)cc(N(c3ccc(C4CC5CCC4C5)cc3)c3ccc([Si](c4ccccc4)(c4ccccc4)c4ccccc4)cc3)cc21 ; CC1(C)c2ccccc2-c2cc(-c3ccccc3)c(N(c3ccc(C4CC5CCC4C5)cc3)c3ccc([Si](c4ccccc4)(c4ccccc4)c4ccccc4)cc3)cc21]

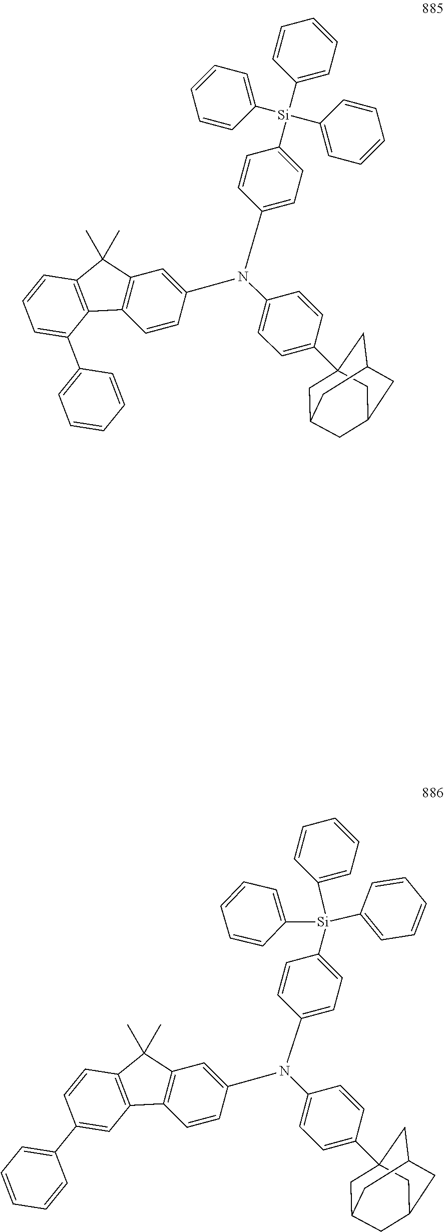 [SMILES: CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3ccccc3)cccc21 ; CC1(C)c2ccc(-c3ccccc3)cc2-c2ccc(N(c3ccc(C45CC6CC(CC(C6)C4)C5)cc3)c3ccc([Si](c4ccccc4)(c4ccccc4)c4ccccc4)cc3)cc21]

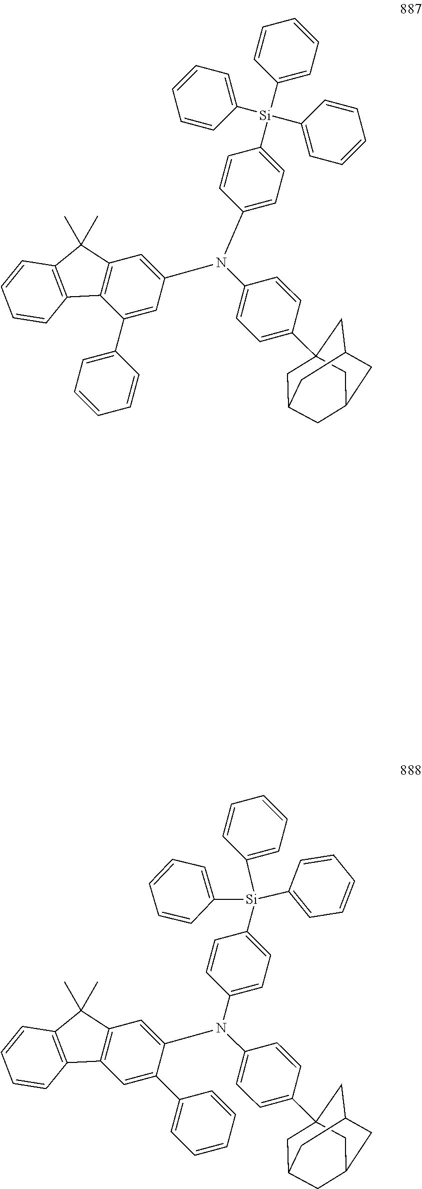 [SMILES: CC1(C)c2ccccc2-c2c(-c3ccccc3)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3ccccc3)c(N(c3ccc(C45CC6CC(CC(C6)C4)C5)cc3)c3ccc([Si](c4ccccc4)(c4ccccc4)c4ccccc4)cc3)cc21]

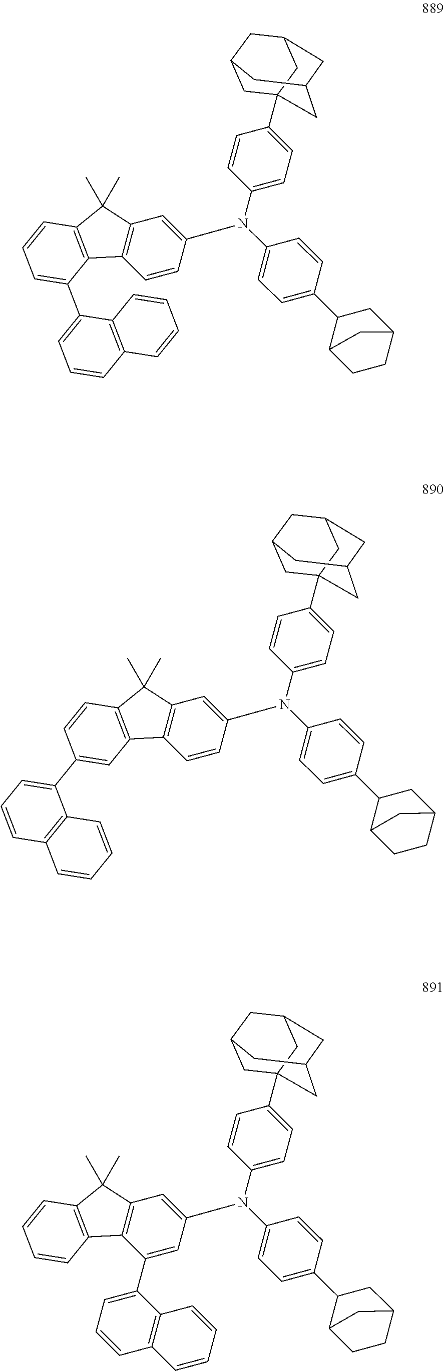 [SMILES: CC1(C)c2cc(N(c3ccc(C4CC5CCC4C5)cc3)c3ccc(C45CC6CC(CC(C6)C4)C5)cc3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3cccc4ccccc34)cc(N(c3ccc(C4CC5CCC4C5)cc3)c3ccc(C45CC6CC(CC(C6)C4)C5)cc3)cc21]

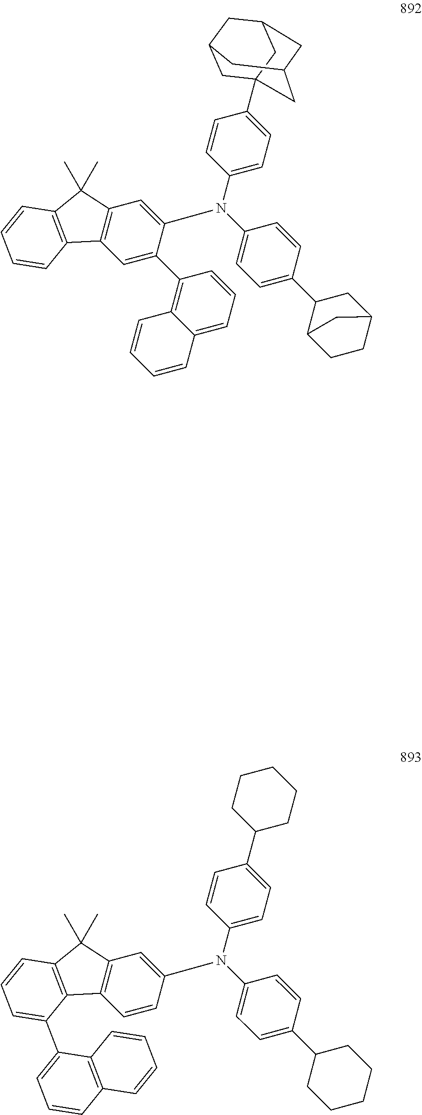 [SMILES: CC1(C)c2ccccc2-c2cc(-c3cccc4ccccc34)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C4CCCCC4)cc3)ccc2-c2c(-c3cccc4ccccc34)cccc21]

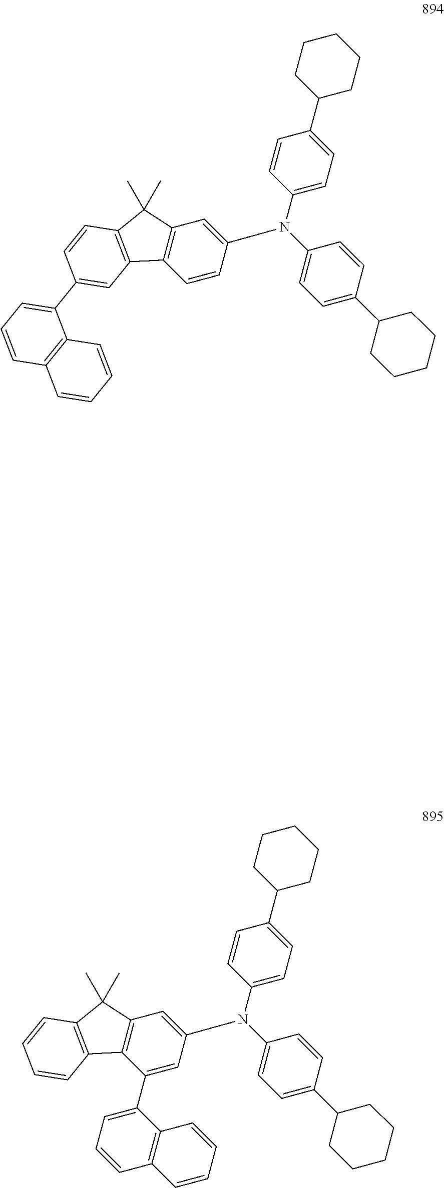 [SMILES: CC1(C)c2ccc(-c3cccc4ccccc34)cc2-c2ccc(N(c3ccc(C4CCCCC4)cc3)c3ccc(C4CCCCC4)cc3)cc21 ; CC1(C)c2ccccc2-c2c(-c3cccc4ccccc34)cc(N(c3ccc(C4CCCCC4)cc3)c3ccc(C4CCCCC4)cc3)cc21]

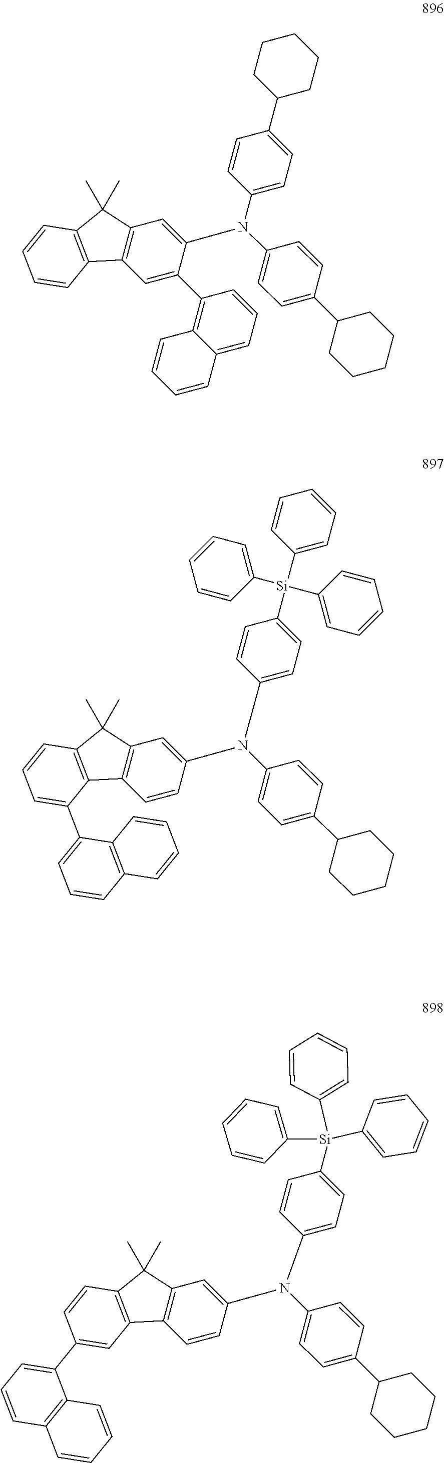 [SMILES: CC1(C)c2ccccc2-c2cc(-c3cccc4ccccc34)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc([Si](c4ccccc4)(c4ccccc4)c4ccccc4)cc3)cc21]

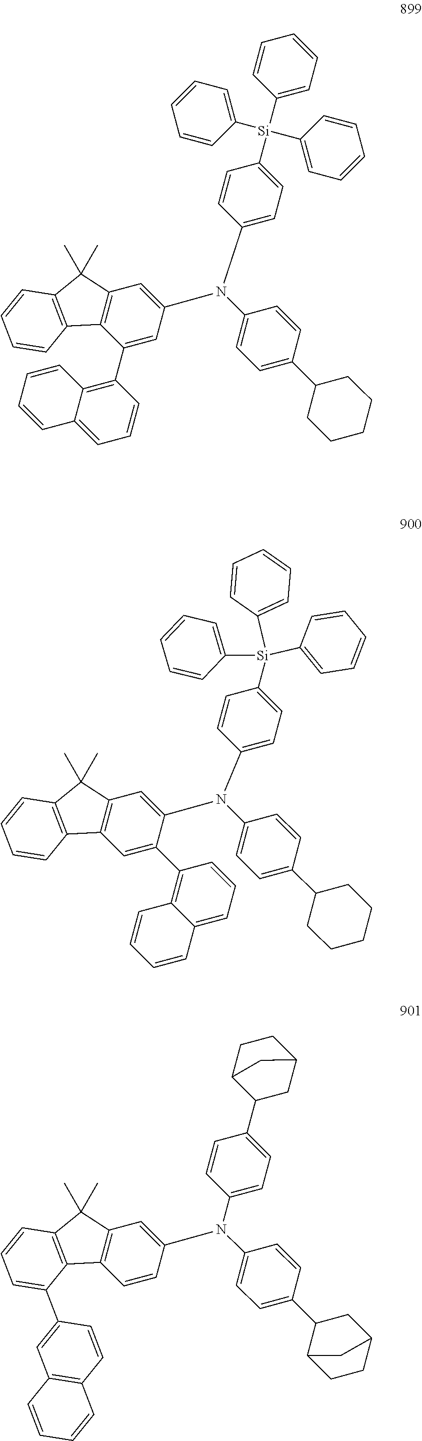 [SMILES: CC1(C)c2ccccc2-c2c(-c3cccc4ccccc34)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3cccc4ccccc34)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3ccc4ccccc4c3)cccc21]

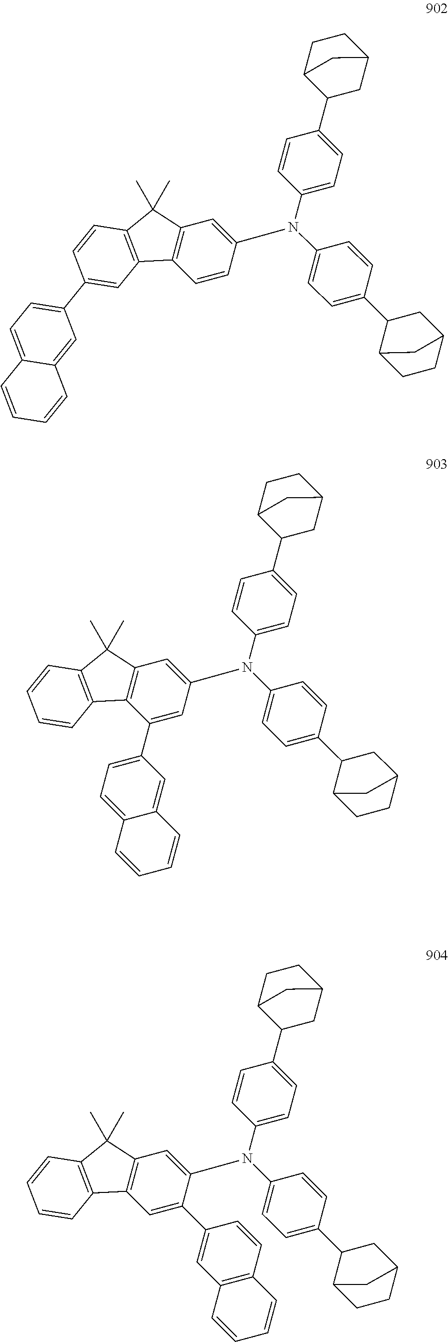 [SMILES: CC1(C)c2ccc(-c3ccc4ccccc4c3)cc2-c2ccc(N(c3ccc(C4CC5CCC4C5)cc3)c3ccc(C4CC5CCC4C5)cc3)cc21 ; CC1(C)c2ccccc2-c2c(-c3ccc4ccccc4c3)cc(N(c3ccc(C4CC5CCC4C5)cc3)c3ccc(C4CC5CCC4C5)cc3)cc21 ; CC1(C)c2ccccc2-c2cc(-c3ccc4ccccc4c3)c(N(c3ccc(C4CC5CCC4C5)cc3)c3ccc(C4CC5CCC4C5)cc3)cc21]

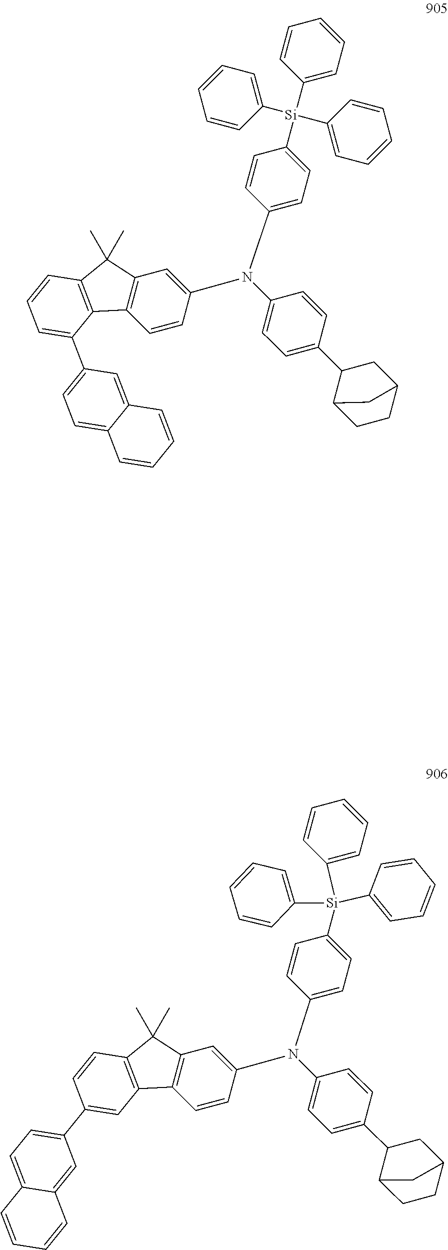 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccc([Si](c4ccccc4)(c4ccccc4)c4ccccc4)cc3)cc21]

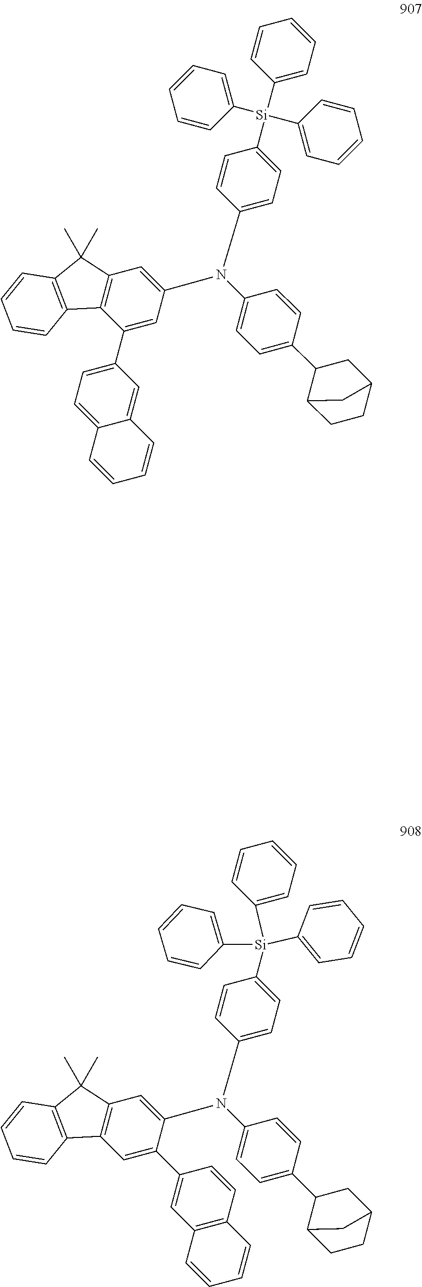 [SMILES: CC1(C)c2ccccc2-c2c(-c3ccc4ccccc4c3)cc(N(c3ccc(C4CC5CCC4C5)cc3)c3ccc([Si](c4ccccc4)(c4ccccc4)c4ccccc4)cc3)cc21 ; CC1(C)c2ccccc2-c2cc(-c3ccc4ccccc4c3)c(N(c3ccc(C4CC5CCC4C5)cc3)c3ccc([Si](c4ccccc4)(c4ccccc4)c4ccccc4)cc3)cc21]

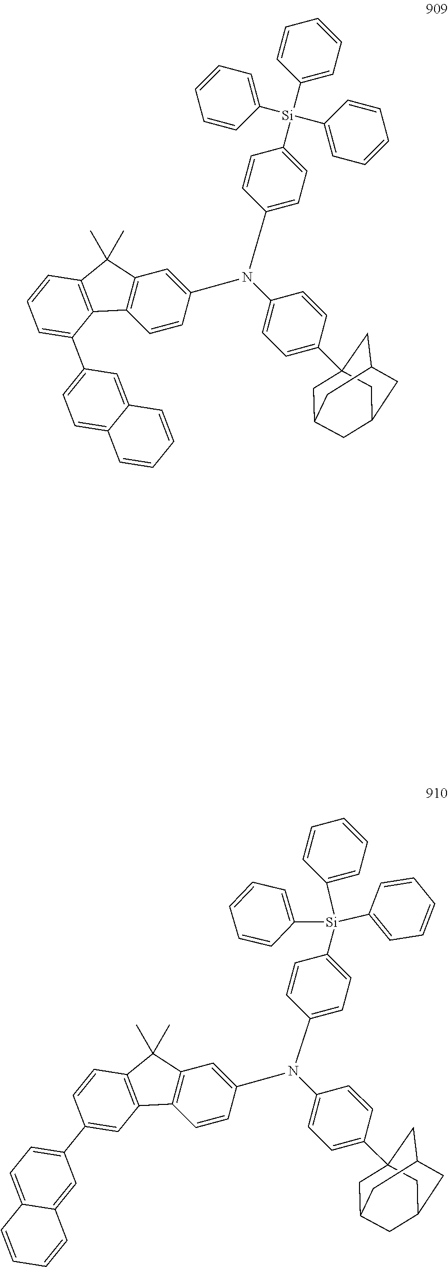 [SMILES: CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C45CC6CC(CC(C6)C4)C5)cc3)c3ccc([Si](c4ccccc4)(c4ccccc4)c4ccccc4)cc3)cc21]

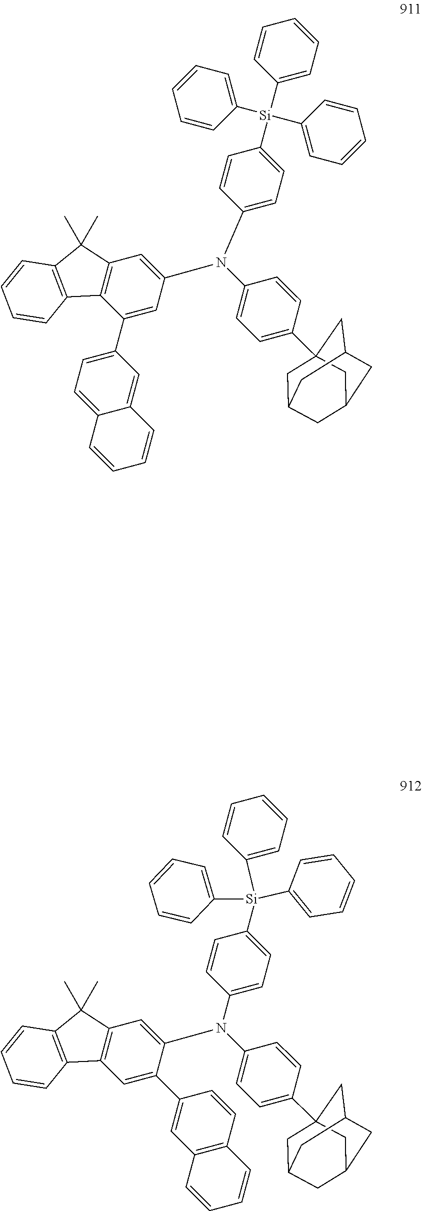 [SMILES: CC1(C)c2ccccc2-c2c(-c3ccc4ccccc4c3)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3ccc4ccccc4c3)c(N(c3ccc(C45CC6CC(CC(C6)C4)C5)cc3)c3ccc([Si](c4ccccc4)(c4ccccc4)c4ccccc4)cc3)cc21]

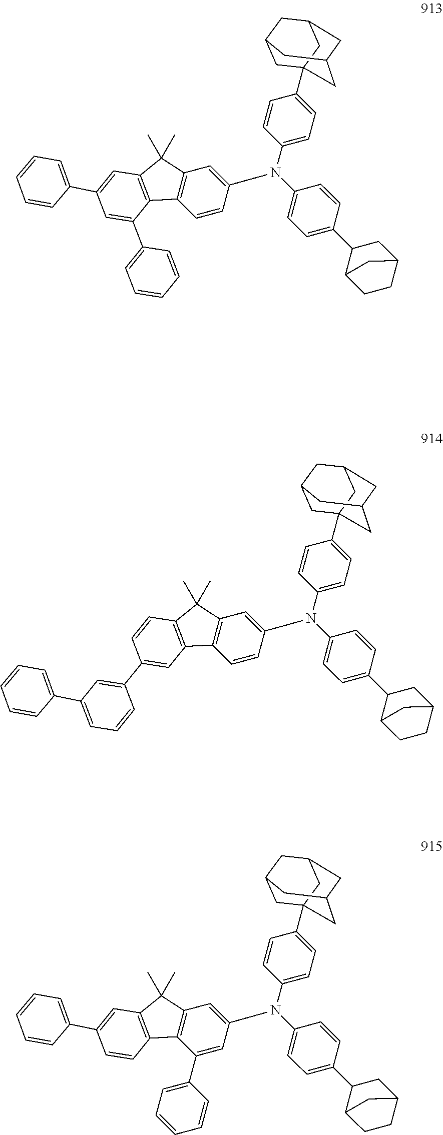 [SMILES: CC1(C)c2cc(N(c3ccc(C4CC5CCC4C5)cc3)c3ccc(C45CC6CC(CC(C6)C4)C5)cc3)ccc2-c2c(-c3ccccc3)cc(-c3ccccc3)cc21 ; CC1(C)c2ccc(-c3cccc(-c4ccccc4)c3)cc2-c2ccc(N(c3ccc(C4CC5CCC4C5)cc3)c3ccc(C45CC6CC(CC(C6)C4)C5)cc3)cc21 ; CC1(C)c2cc(-c3ccccc3)ccc2-c2c(-c3ccccc3)cc(N(c3ccc(C4CC5CCC4C5)cc3)c3ccc(C45CC6CC(CC(C6)C4)C5)cc3)cc21]

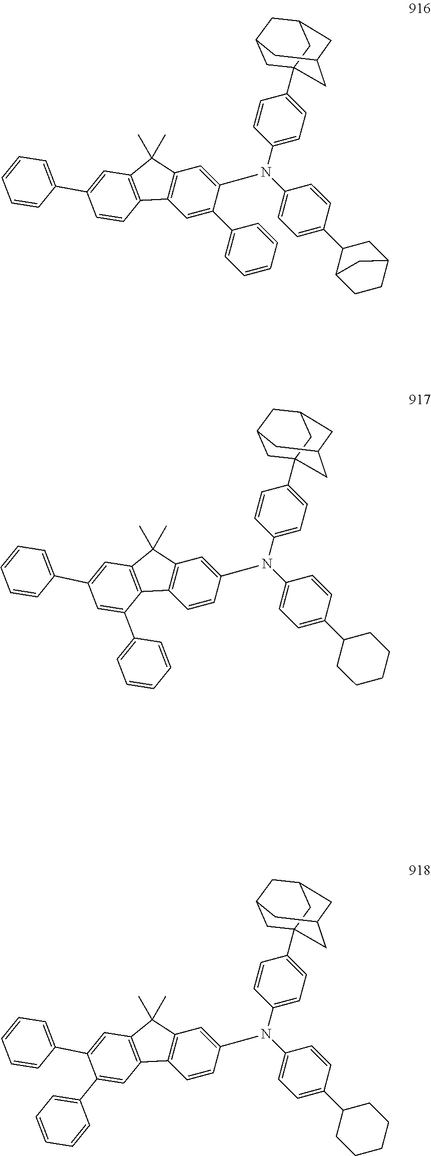 [SMILES: CC1(C)c2cc(-c3ccccc3)ccc2-c2cc(-c3ccccc3)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccccc3)cc(-c3ccccc3)cc21 ; CC1(C)c2cc(N(c3ccc(C4CCCCC4)cc3)c3ccc(C45CC6CC(CC(C6)C4)C5)cc3)ccc2-c2cc(-c3ccccc3)c(-c3ccccc3)cc21]

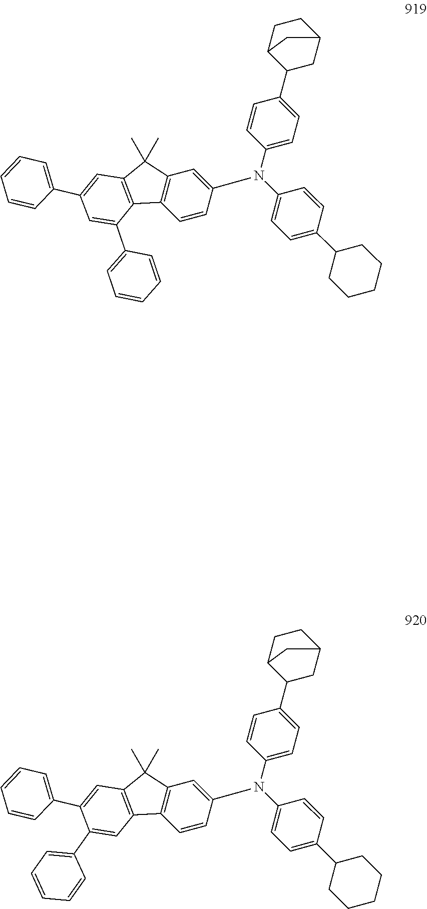 [SMILES: CC1(C)c2cc(N(c3ccc(C4CCCCC4)cc3)c3ccc(C4CC5CCC4C5)cc3)ccc2-c2c(-c3ccccc3)cc(-c3ccccc3)cc21 ; CC1(C)c2cc(N(c3ccc(C4CCCCC4)cc3)c3ccc(C4CC5CCC4C5)cc3)ccc2-c2cc(-c3ccccc3)c(-c3ccccc3)cc21]

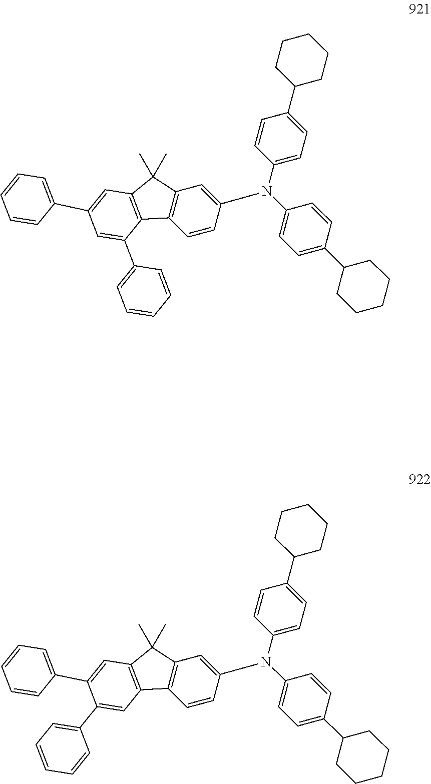 [SMILES: CC1(C)c2cc(N(c3ccc(C4CCCCC4)cc3)c3ccc(C4CCCCC4)cc3)ccc2-c2c(-c3ccccc3)cc(-c3ccccc3)cc21 ; CC1(C)c2cc(N(c3ccc(C4CCCCC4)cc3)c3ccc(C4CCCCC4)cc3)ccc2-c2cc(-c3ccccc3)c(-c3ccccc3)cc21]

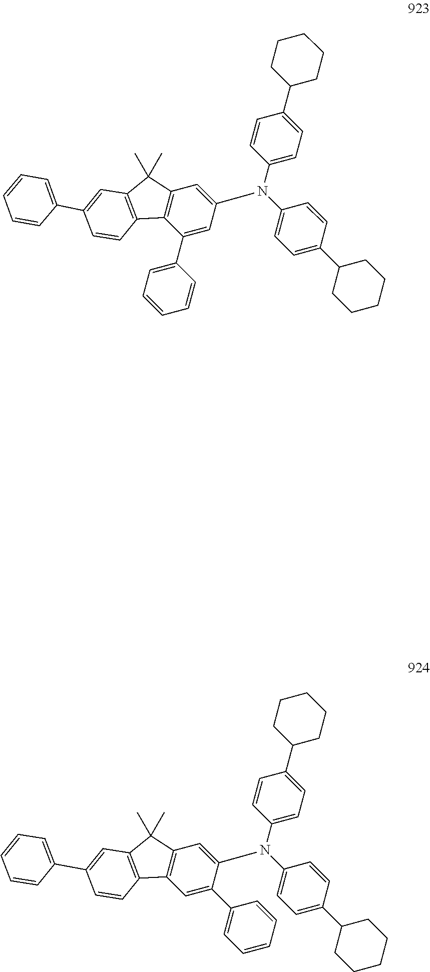 [SMILES: CC1(C)c2cc(-c3ccccc3)ccc2-c2c(-c3ccccc3)cc(N(c3ccc(C4CCCCC4)cc3)c3ccc(C4CCCCC4)cc3)cc21 ; CC1(C)c2cc(-c3ccccc3)ccc2-c2cc(-c3ccccc3)c(N(c3ccc(C4CCCCC4)cc3)c3ccc(C4CCCCC4)cc3)cc21]

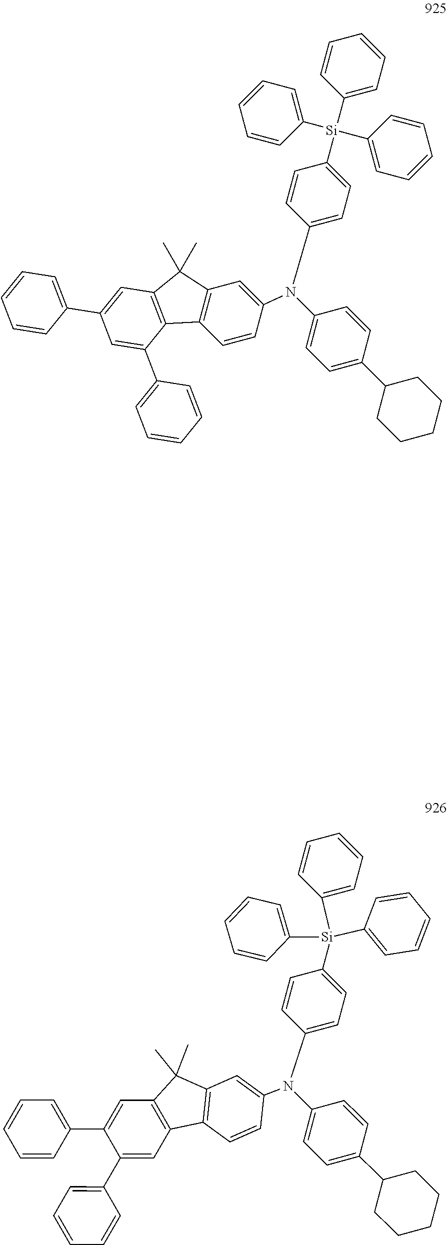 [SMILES: CC1(C)c2cc(N(c3ccc(C4CCCCC4)cc3)c3ccc([Si](c4ccccc4)(c4ccccc4)c4ccccc4)cc3)ccc2-c2c(-c3ccccc3)cc(-c3ccccc3)cc21 ; CC1(C)c2cc(N(c3ccc(C4CCCCC4)cc3)c3ccc([Si](c4ccccc4)(c4ccccc4)c4ccccc4)cc3)ccc2-c2cc(-c3ccccc3)c(-c3ccccc3)cc21]

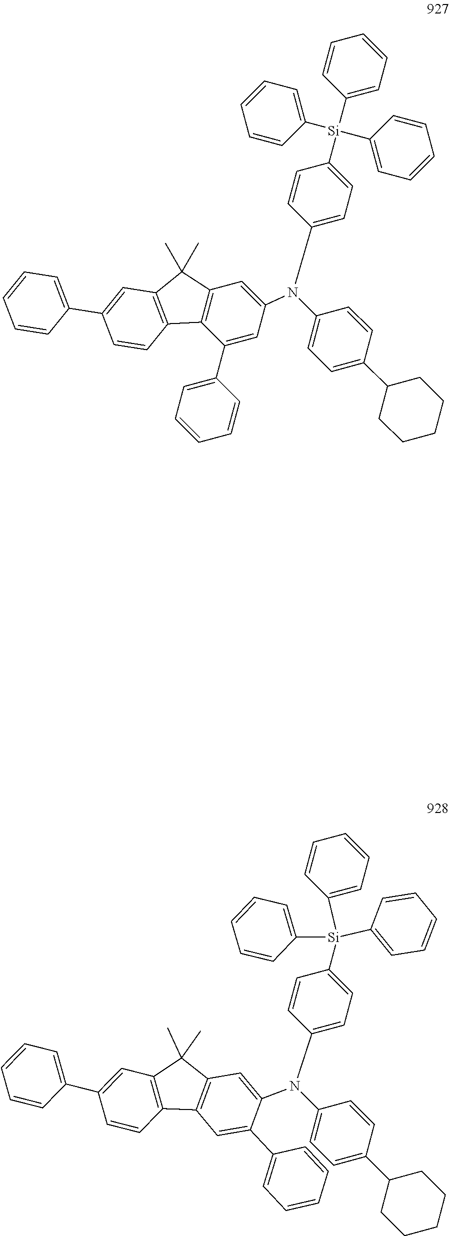 [SMILES: CC1(C)c2cc(-c3ccccc3)ccc2-c2c(-c3ccccc3)cc(N(c3ccc(C4CCCCC4)cc3)c3ccc([Si](c4ccccc4)(c4ccccc4)c4ccccc4)cc3)cc21 ; CC1(C)c2cc(-c3ccccc3)ccc2-c2cc(-c3ccccc3)c(N(c3ccc(C4CCCCC4)cc3)c3ccc([Si](c4ccccc4)(c4ccccc4)c4ccccc4)cc3)cc21]

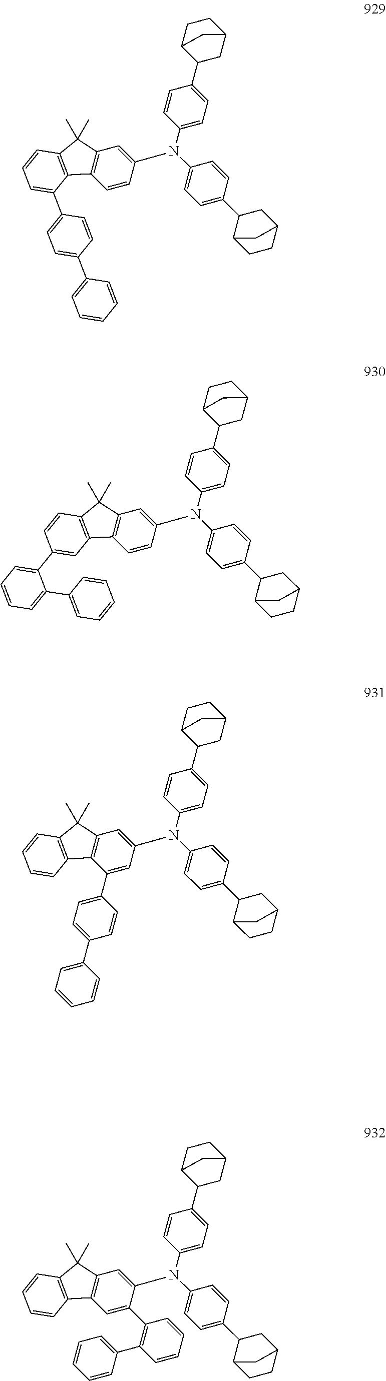 [SMILES: CC1(C)c2cc(N(c3ccc(C4CC5CCC4C5)cc3)c3ccc(C4CC5CCC4C5)cc3)ccc2-c2c(-c3ccc(-c4ccccc4)cc3)cccc21 ; CC1(C)c2ccc(-c3ccccc3-c3ccccc3)cc2-c2ccc(N(c3ccc(C4CC5CCC4C5)cc3)c3ccc(C4CC5CCC4C5)cc3)cc21 ; CC1(C)c2ccccc2-c2c(-c3ccc(-c4ccccc4)cc3)cc(N(c3ccc(C4CC5CCC4C5)cc3)c3ccc(C4CC5CCC4C5)cc3)cc21 ; CC1(C)c2ccccc2-c2cc(-c3ccccc3-c3ccccc3)c(N(c3ccc(C4CC5CCC4C5)cc3)c3ccc(C4CC5CCC4C5)cc3)cc21]

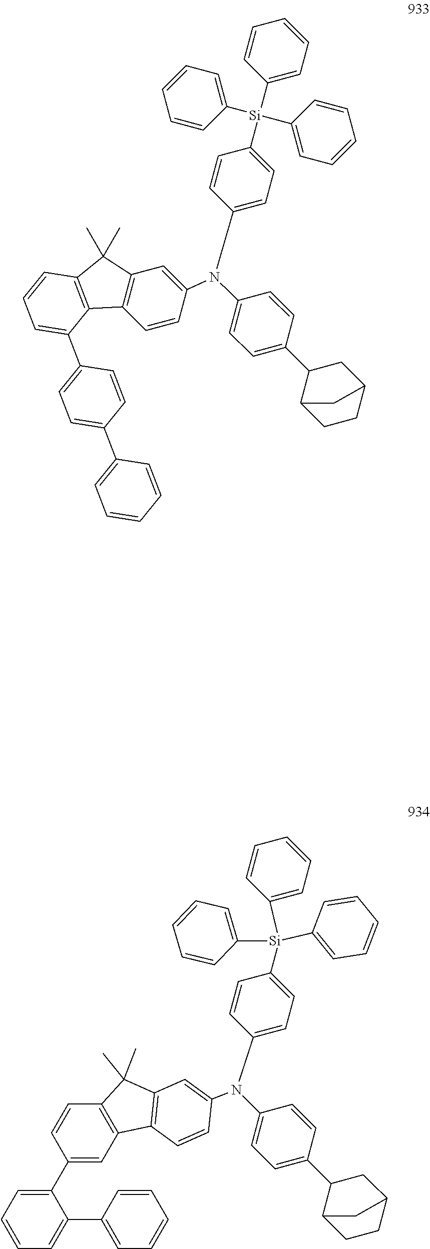 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3ccc(-c4ccccc4)cc3)cccc21 ; CC1(C)c2ccc(-c3ccccc3-c3ccccc3)cc2-c2ccc(N(c3ccc(C4CC5CCC4C5)cc3)c3ccc([Si](c4ccccc4)(c4ccccc4)c4ccccc4)cc3)cc21]

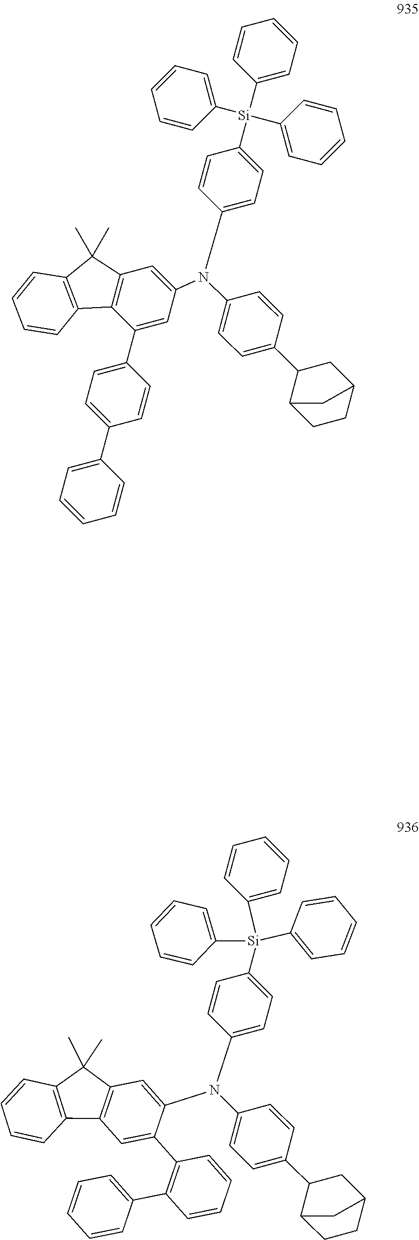 [SMILES: CC1(C)c2ccccc2-c2c(-c3ccc(-c4ccccc4)cc3)cc(N(c3ccc(C4CC5CCC4C5)cc3)c3ccc([Si](c4ccccc4)(c4ccccc4)c4ccccc4)cc3)cc21 ; CC1(C)c2ccccc2-c2cc(-c3ccccc3-c3ccccc3)c(N(c3ccc(C4CC5CCC4C5)cc3)c3ccc([Si](c4ccccc4)(c4ccccc4)c4ccccc4)cc3)cc21]

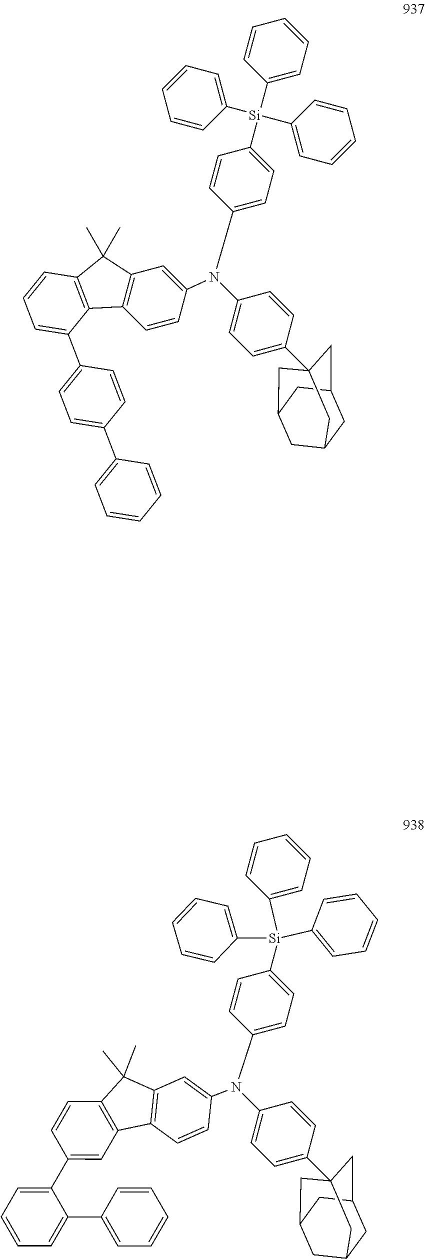 [SMILES: CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3ccc(-c4ccccc4)cc3)cccc21 ; CC1(C)c2ccc(-c3ccccc3-c3ccccc3)cc2-c2ccc(N(c3ccc(C45CC6CC(CC(C6)C4)C5)cc3)c3ccc([Si](c4ccccc4)(c4ccccc4)c4ccccc4)cc3)cc21]

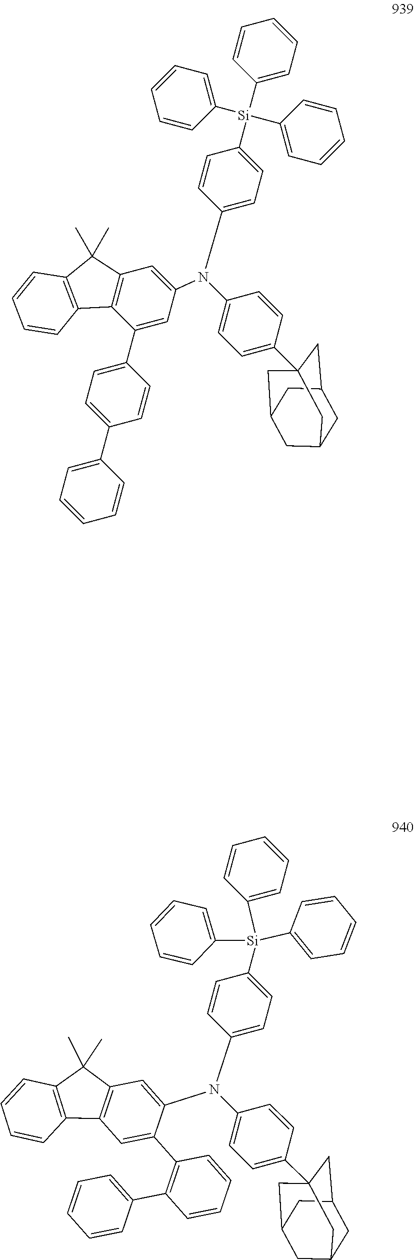 [SMILES: CC1(C)c2ccccc2-c2c(-c3ccc(-c4ccccc4)cc3)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3ccccc3-c3ccccc3)c(N(c3ccc(C45CC6CC(CC(C6)C4)C5)cc3)c3ccc([Si](c4ccccc4)(c4ccccc4)c4ccccc4)cc3)cc21]

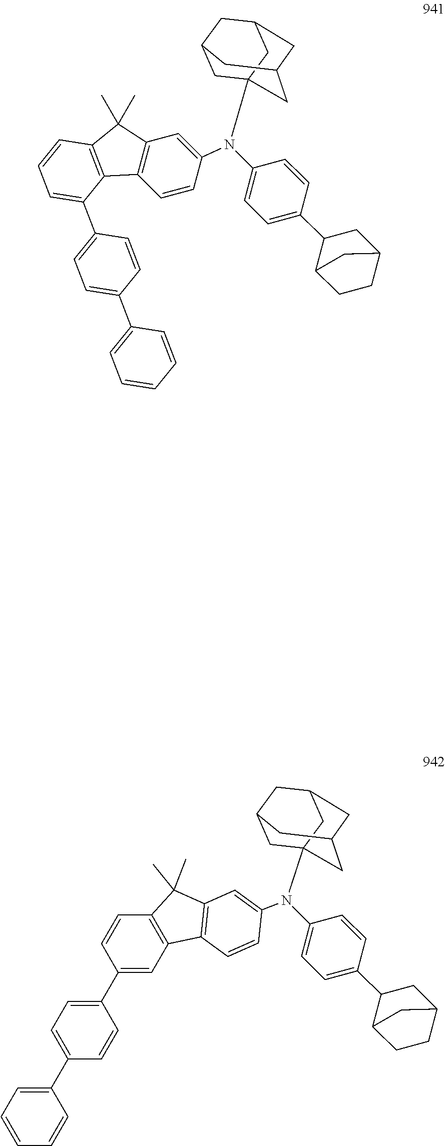 [SMILES: CC1(C)c2cc(N(c3ccc(C4CC5CCC4C5)cc3)C34CC5CC(CC(C5)C3)C4)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)C34CC5CC(CC(C5)C3)C4)cc21]

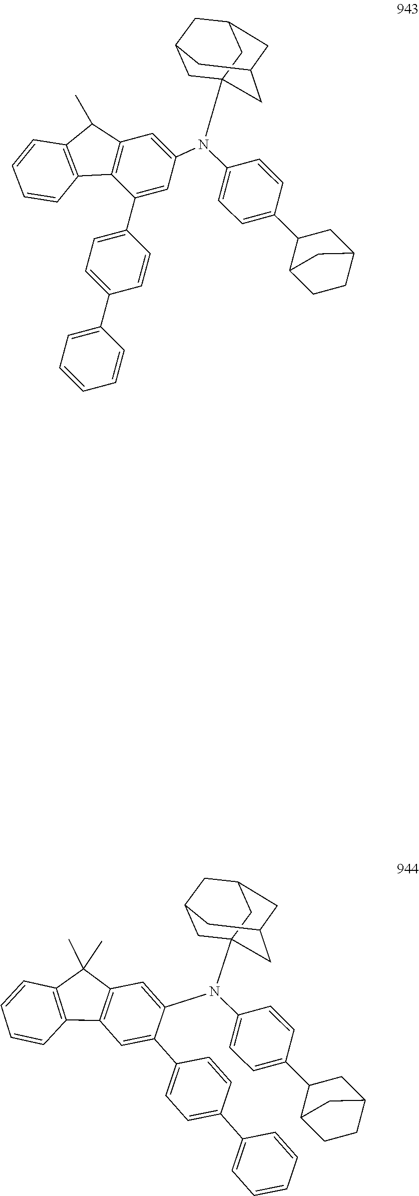 [SMILES: CC1c2ccccc2-c2c(-c3ccc(-c4ccccc4)cc3)cc(N(c3ccc(C4CC5CCC4C5)cc3)C34CC5CC(CC(C5)C3)C4)cc21 ; CC1(C)c2ccccc2-c2cc(-c3ccc(-c4ccccc4)cc3)c(N(c3ccc(C4CC5CCC4C5)cc3)C34CC5CC(CC(C5)C3)C4)cc21]

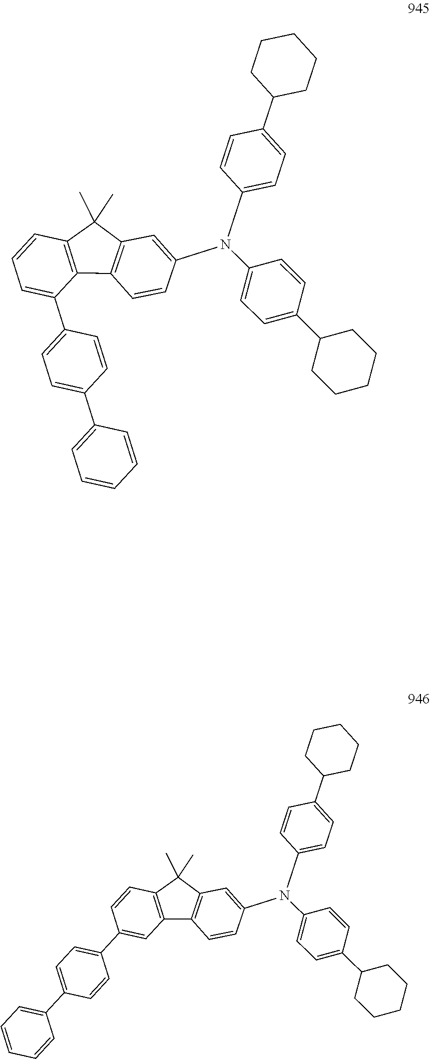 [SMILES: CC1(C)c2cc(N(c3ccc(C4CCCCC4)cc3)c3ccc(C4CCCCC4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4CCCCC4)cc3)cc21]

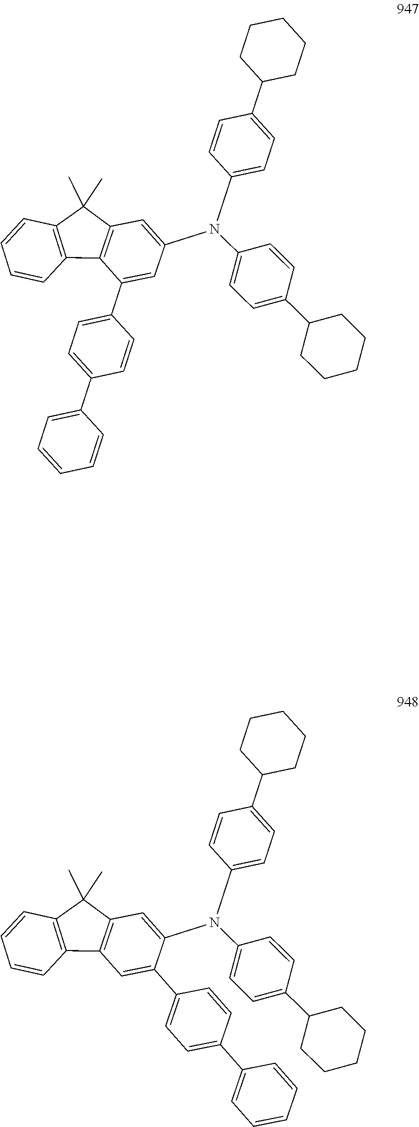 [SMILES: CC1(C)c2ccccc2-c2c(-c3ccc(-c4ccccc4)cc3)cc(N(c3ccc(C4CCCCC4)cc3)c3ccc(C4CCCCC4)cc3)cc21 ; CC1(C)c2ccccc2-c2cc(-c3ccc(-c4ccccc4)cc3)c(N(c3ccc(C4CCCCC4)cc3)c3ccc(C4CCCCC4)cc3)cc21]

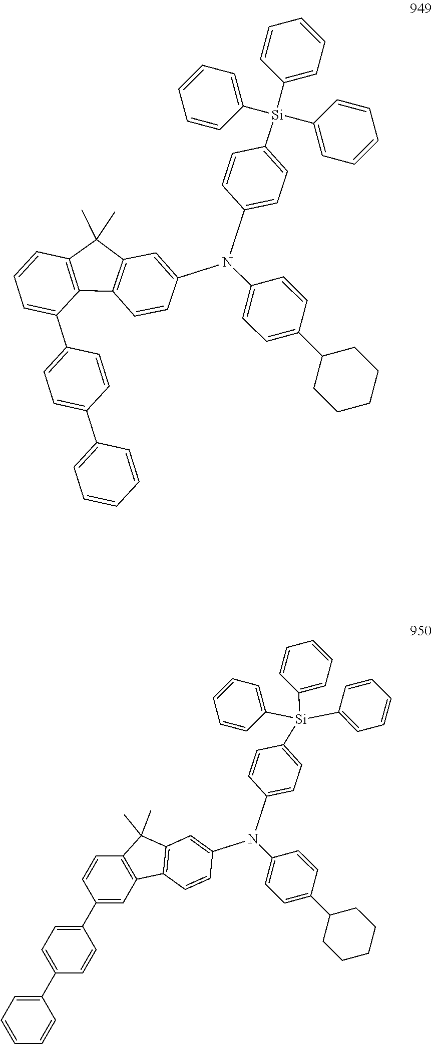 [SMILES: CC1(C)c2cc(N(c3ccc(C4CCCCC4)cc3)c3ccc([Si](c4ccccc4)(c4ccccc4)c4ccccc4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc([Si](c4ccccc4)(c4ccccc4)c4ccccc4)cc3)cc21]

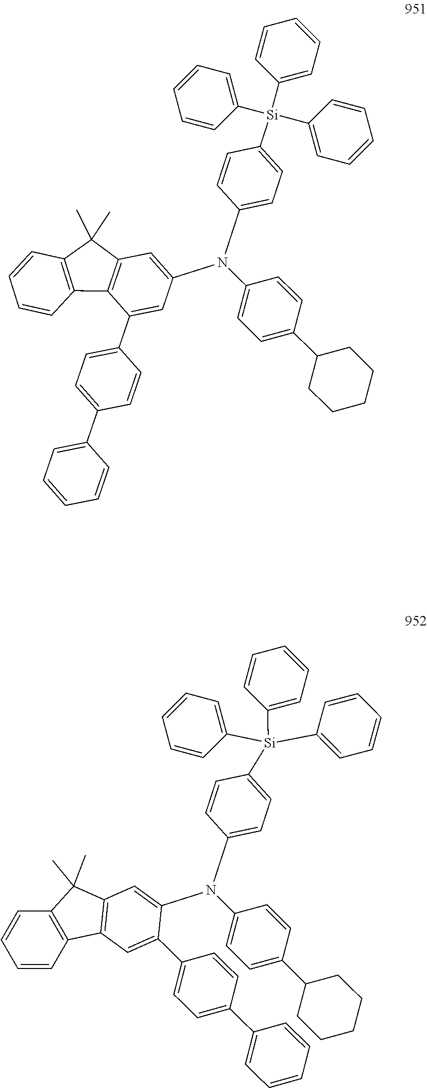 [SMILES: CC1(C)c2ccccc2-c2c(-c3ccc(-c4ccccc4)cc3)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3ccc(-c4ccccc4)cc3)c(N(c3ccc(C4CCCCC4)cc3)c3ccc([Si](c4ccccc4)(c4ccccc4)c4ccccc4)cc3)cc21]

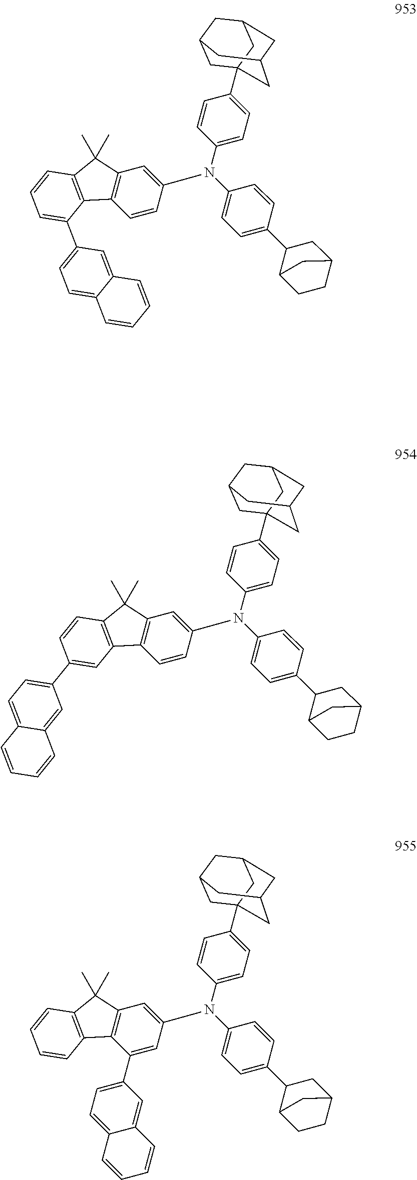 [SMILES: CC1(C)c2cc(N(c3ccc(C4CC5CCC4C5)cc3)c3ccc(C45CC6CC(CC(C6)C4)C5)cc3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3ccc4ccccc4c3)cc(N(c3ccc(C4CC5CCC4C5)cc3)c3ccc(C45CC6CC(CC(C6)C4)C5)cc3)cc21]

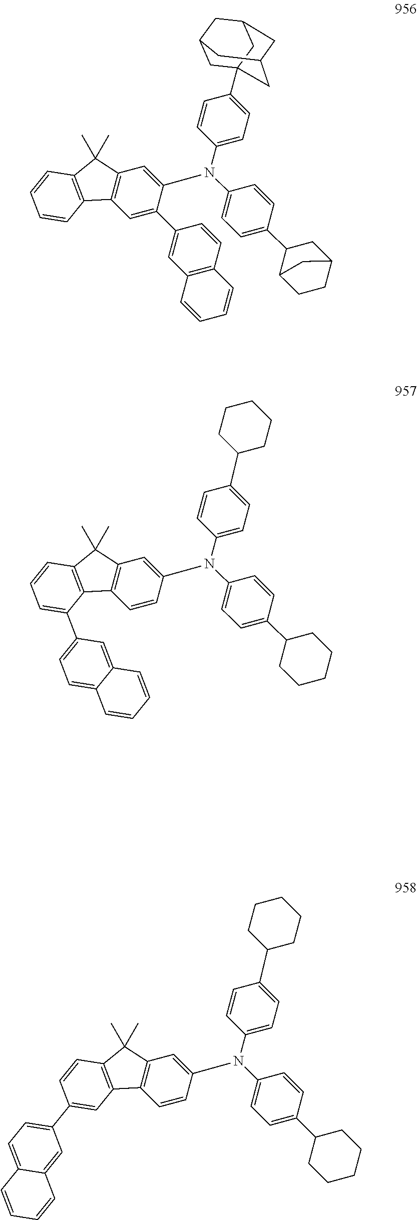 [SMILES: CC1(C)c2ccccc2-c2cc(-c3ccc4ccccc4c3)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C4CCCCC4)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CCCCC4)cc3)cc21]

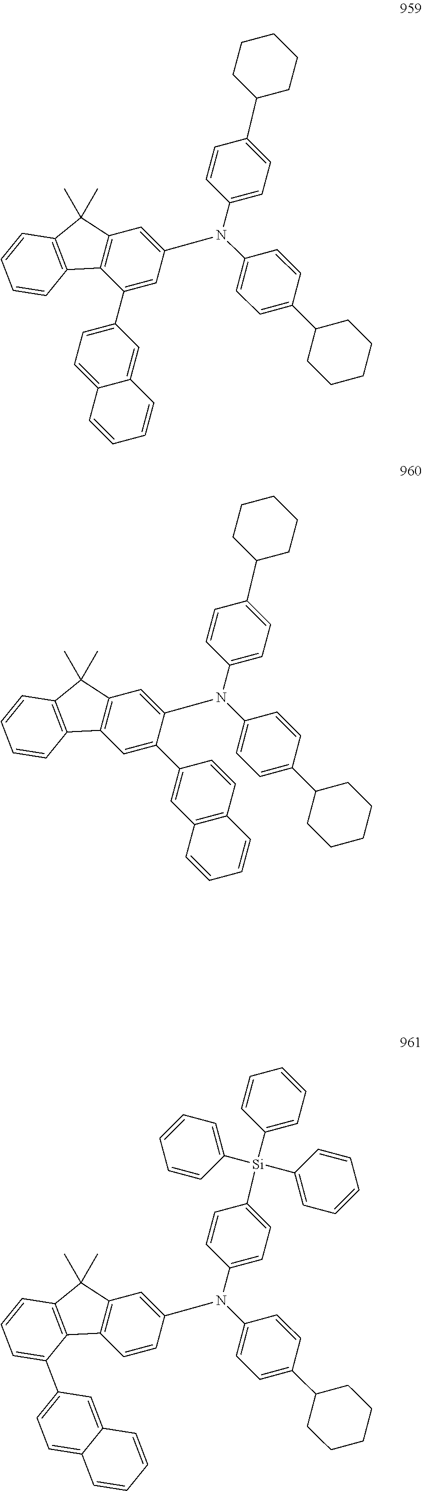 [SMILES: CC1(C)c2ccccc2-c2c(-c3ccc4ccccc4c3)cc(N(c3ccc(C4CCCCC4)cc3)c3ccc(C4CCCCC4)cc3)cc21 ; CC1(C)c2ccccc2-c2cc(-c3ccc4ccccc4c3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccc4ccccc4c3)cccc21]

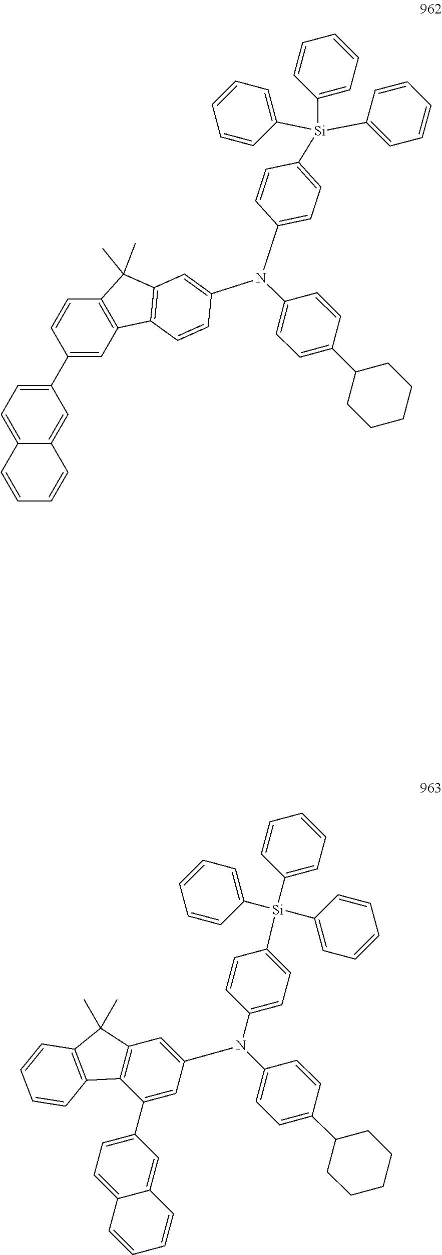 [SMILES: CC1(C)c2ccc(-c3ccc4ccccc4c3)cc2-c2ccc(N(c3ccc(C4CCCCC4)cc3)c3ccc([Si](c4ccccc4)(c4ccccc4)c4ccccc4)cc3)cc21 ; CC1(C)c2ccccc2-c2c(-c3ccc4ccccc4c3)cc(N(c3ccc(C4CCCCC4)cc3)c3ccc([Si](c4ccccc4)(c4ccccc4)c4ccccc4)cc3)cc21]

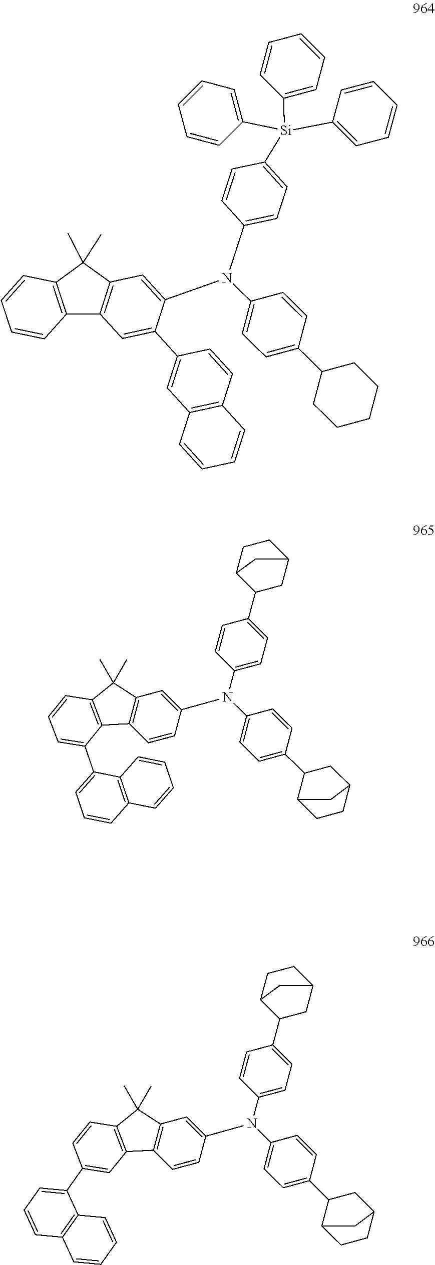 [SMILES: CC1(C)c2ccccc2-c2cc(-c3ccc4ccccc4c3)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccc(C4CC5CCC4C5)cc3)cc21]

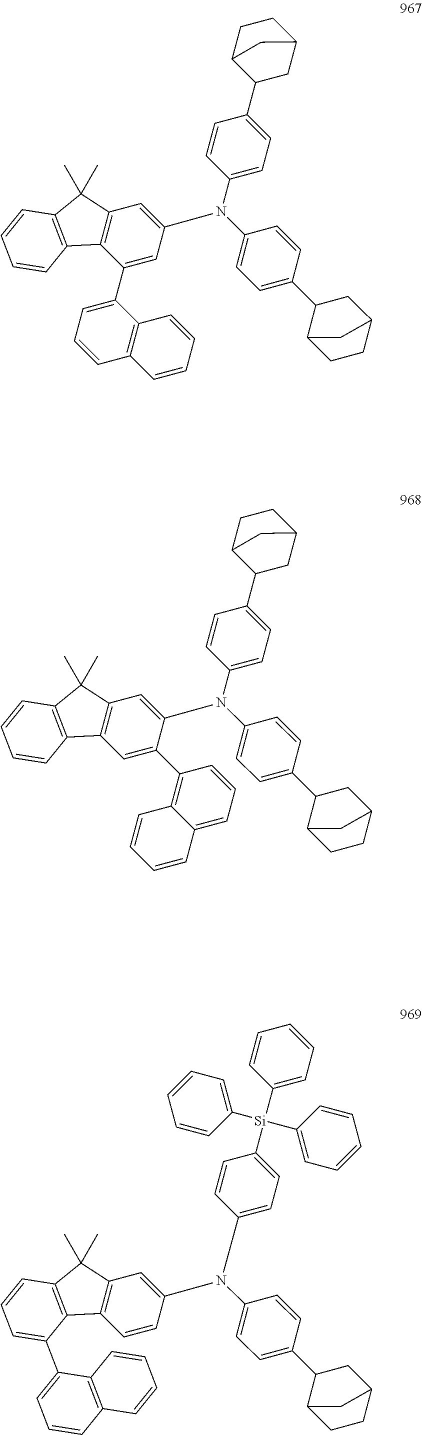 [SMILES: CC1(C)c2ccccc2-c2c(-c3cccc4ccccc34)cc(N(c3ccc(C4CC5CCC4C5)cc3)c3ccc(C4CC5CCC4C5)cc3)cc21 ; CC1(C)c2ccccc2-c2cc(-c3cccc4ccccc34)c(N(c3ccc(C4CC5CCC4C5)cc3)c3ccc(C4CC5CCC4C5)cc3)cc21 ; CC1(C)c2cc(N(c3ccc(C4CC5CCC4C5)cc3)c3ccc([Si](c4ccccc4)(c4ccccc4)c4ccccc4)cc3)ccc2-c2c(-c3cccc4ccccc34)cccc21]

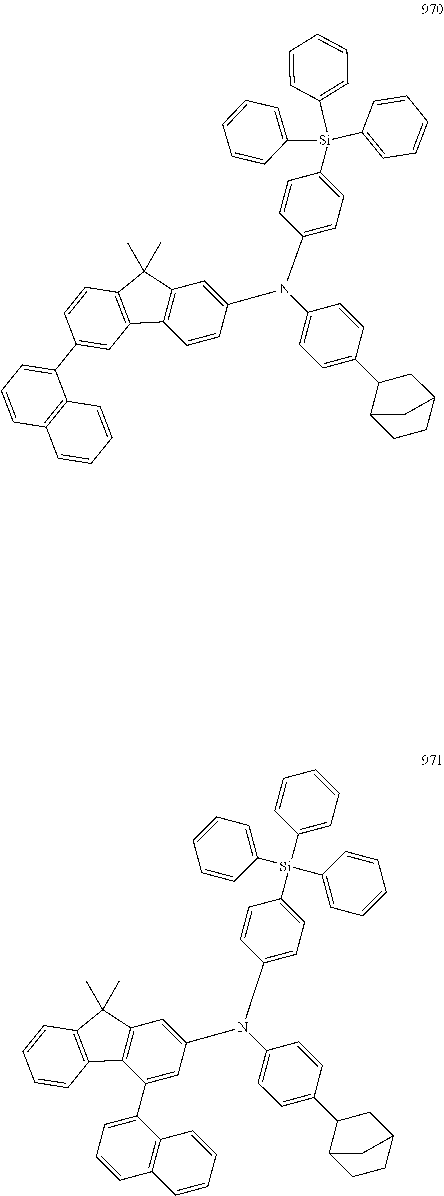 [SMILES: CC1(C)c2ccc(-c3cccc4ccccc34)cc2-c2ccc(N(c3ccc(C4CC5CCC4C5)cc3)c3ccc([Si](c4ccccc4)(c4ccccc4)c4ccccc4)cc3)cc21 ; CC1(C)c2ccccc2-c2c(-c3cccc4ccccc34)cc(N(c3ccc(C4CC5CCC4C5)cc3)c3ccc([Si](c4ccccc4)(c4ccccc4)c4ccccc4)cc3)cc21]

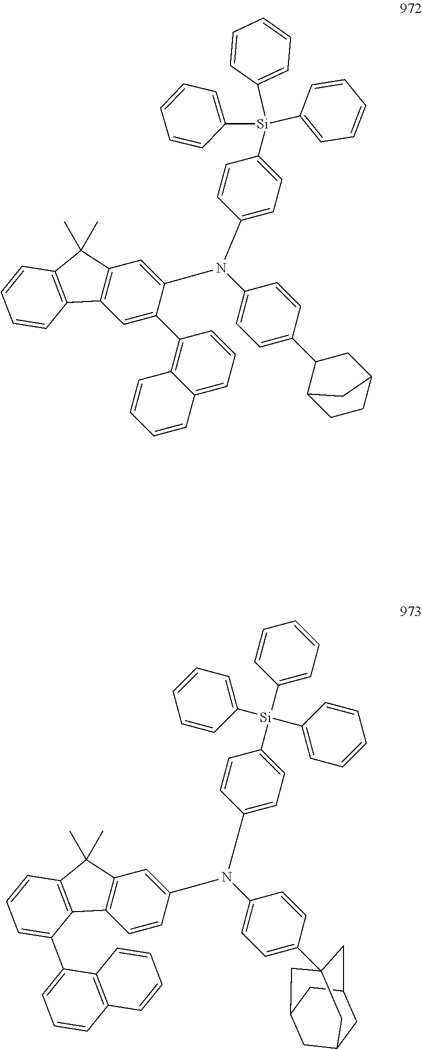 [SMILES: CC1(C)c2ccccc2-c2cc(-c3cccc4ccccc34)c(N(c3ccc(C4CC5CCC4C5)cc3)c3ccc([Si](c4ccccc4)(c4ccccc4)c4ccccc4)cc3)cc21 ; CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3cccc4ccccc34)cccc21]

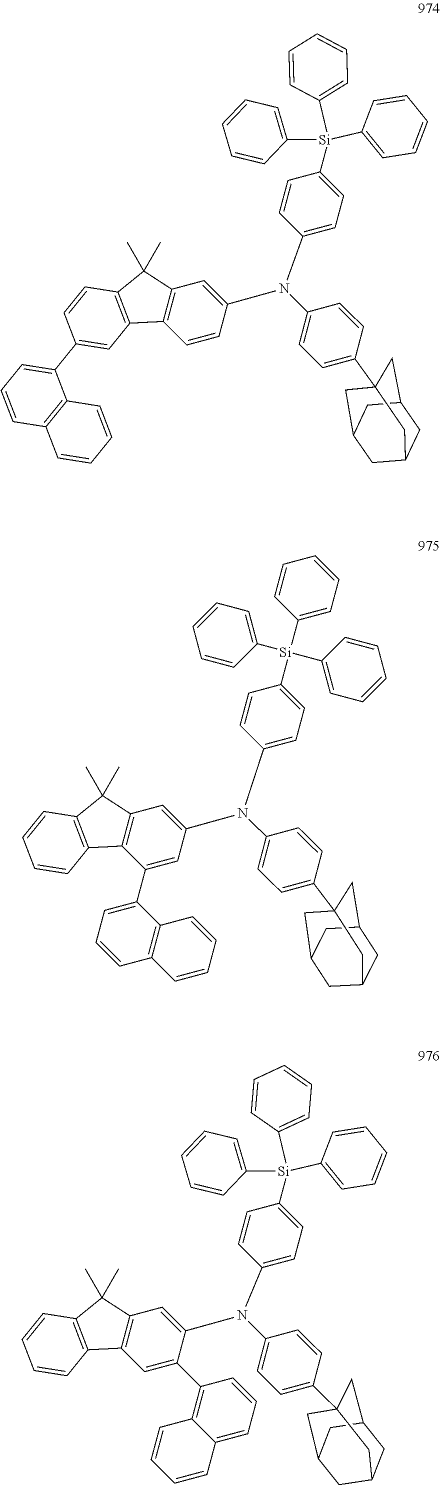 [SMILES: CC1(C)c2ccc(-c3cccc4ccccc34)cc2-c2ccc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2c(-c3cccc4ccccc34)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3cccc4ccccc34)c(N(c3ccc(C45CC6CC(CC(C6)C4)C5)cc3)c3ccc([Si](c4ccccc4)(c4ccccc4)c4ccccc4)cc3)cc21]

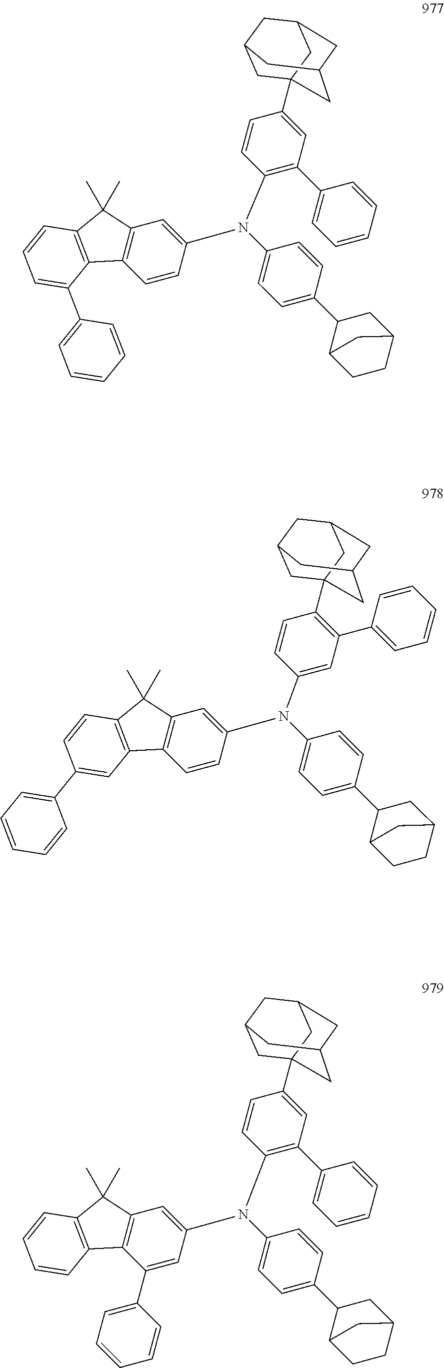 [SMILES: CC1(C)c2cc(N(c3ccc(C4CC5CCC4C5)cc3)c3ccc(C45CC6CC(CC(C6)C4)C5)cc3-c3ccccc3)ccc2-c2c(-c3ccccc3)cccc21 ; CC1(C)c2ccc(-c3ccccc3)cc2-c2ccc(N(c3ccc(C4CC5CCC4C5)cc3)c3ccc(C45CC6CC(CC(C6)C4)C5)c(-c4ccccc4)c3)cc21 ; CC1(C)c2ccccc2-c2c(-c3ccccc3)cc(N(c3ccc(C4CC5CCC4C5)cc3)c3ccc(C45CC6CC(CC(C6)C4)C5)cc3-c3ccccc3)cc21]

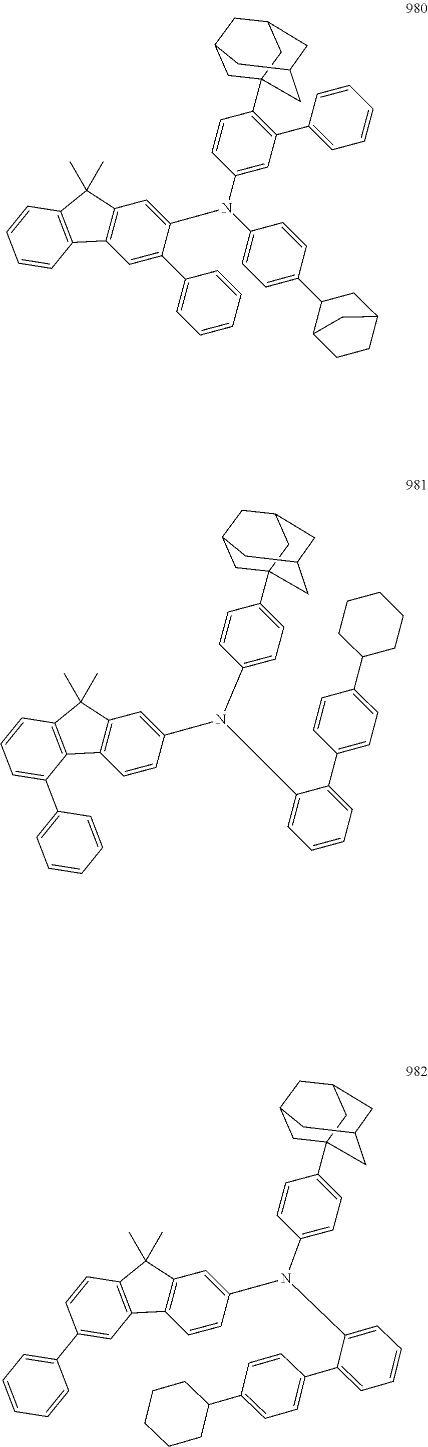 [SMILES: CC1(C)c2ccccc2-c2cc(-c3ccccc3)c(N(c3ccc(C4CC5CCC4C5)cc3)c3ccc(C45CC6CC(CC(C6)C4)C5)c(-c4ccccc4)c3)cc21 ; CC1(C)c2cc(N(c3ccc(C45CC6CC(CC(C6)C4)C5)cc3)c3ccccc3-c3ccc(C4CCCCC4)cc3)ccc2-c2c(-c3ccccc3)cccc21 ; CC1(C)c2ccc(-c3ccccc3)cc2-c2ccc(N(c3ccc(C45CC6CC(CC(C6)C4)C5)cc3)c3ccccc3-c3ccc(C4CCCCC4)cc3)cc21]

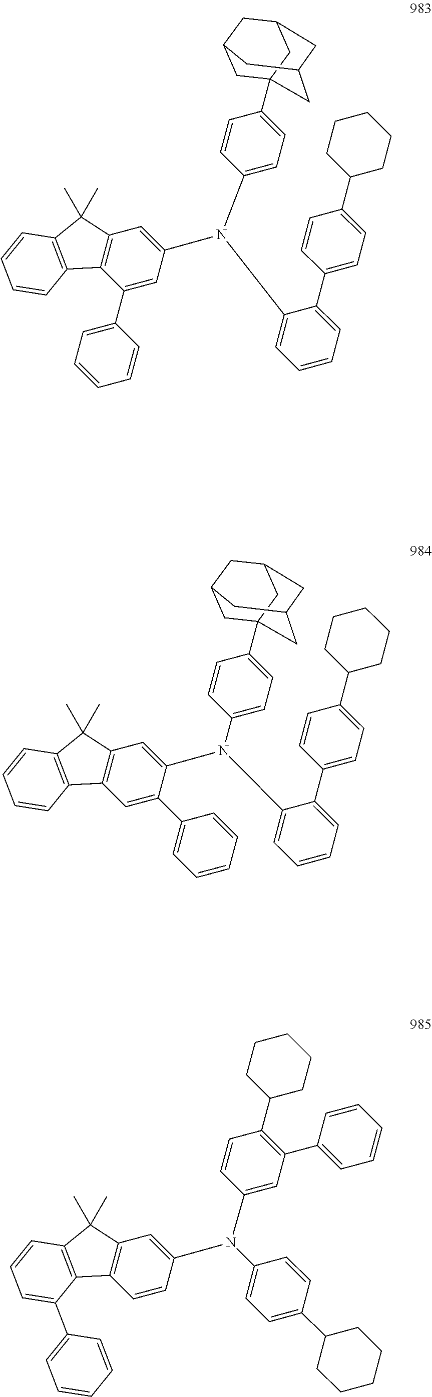 [SMILES: CC1(C)c2ccccc2-c2c(-c3ccccc3)cc(N(c3ccc(C45CC6CC(CC(C6)C4)C5)cc3)c3ccccc3-c3ccc(C4CCCCC4)cc3)cc21 ; CC1(C)c2ccccc2-c2cc(-c3ccccc3)c(N(c3ccc(C45CC6CC(CC(C6)C4)C5)cc3)c3ccccc3-c3ccc(C4CCCCC4)cc3)cc21 ; CC1(C)c2cc(N(c3ccc(C4CCCCC4)cc3)c3ccc(C4CCCCC4)c(-c4ccccc4)c3)ccc2-c2c(-c3ccccc3)cccc21]

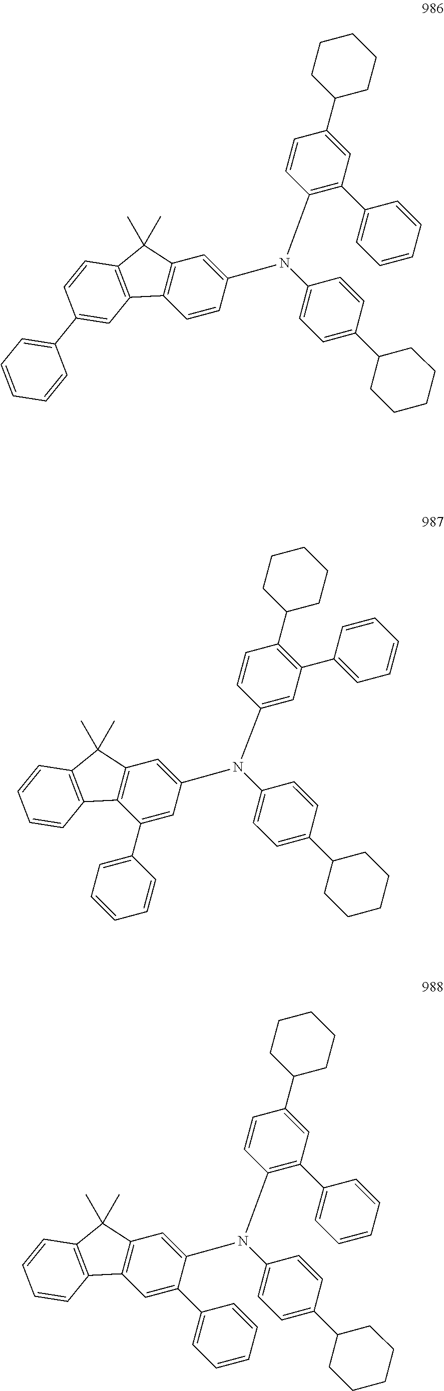

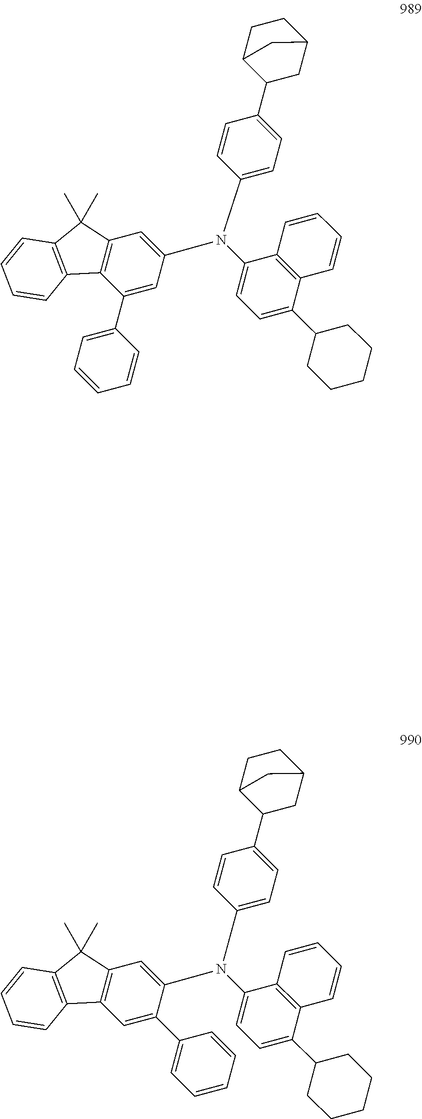

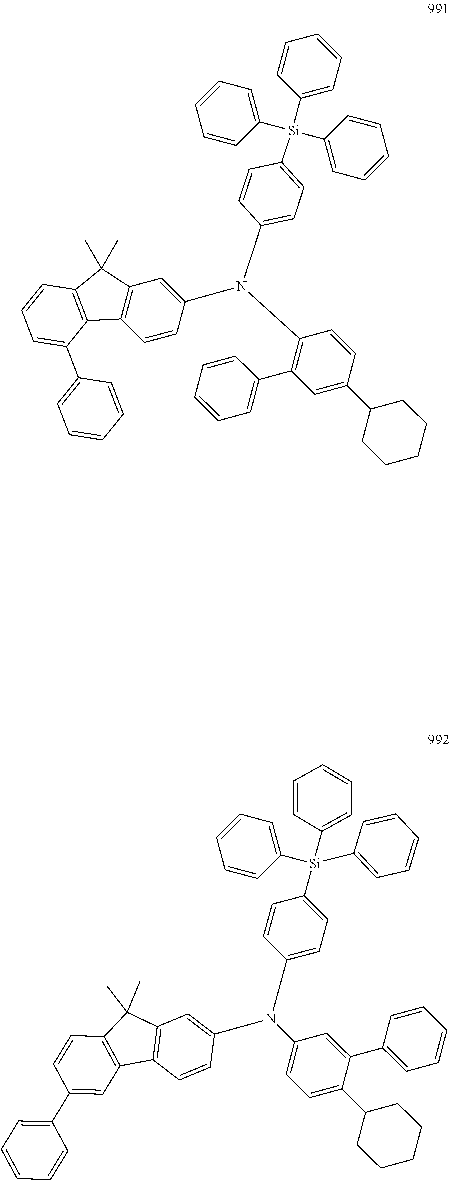

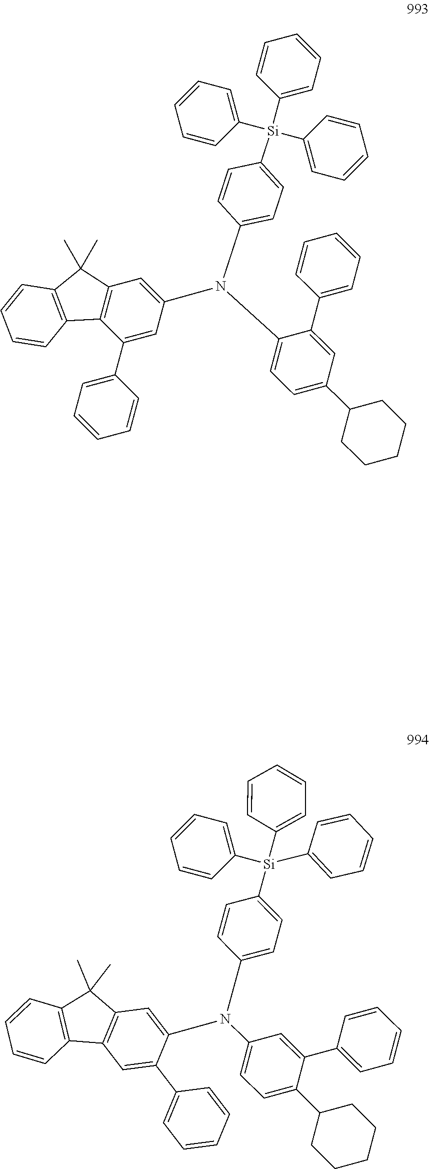

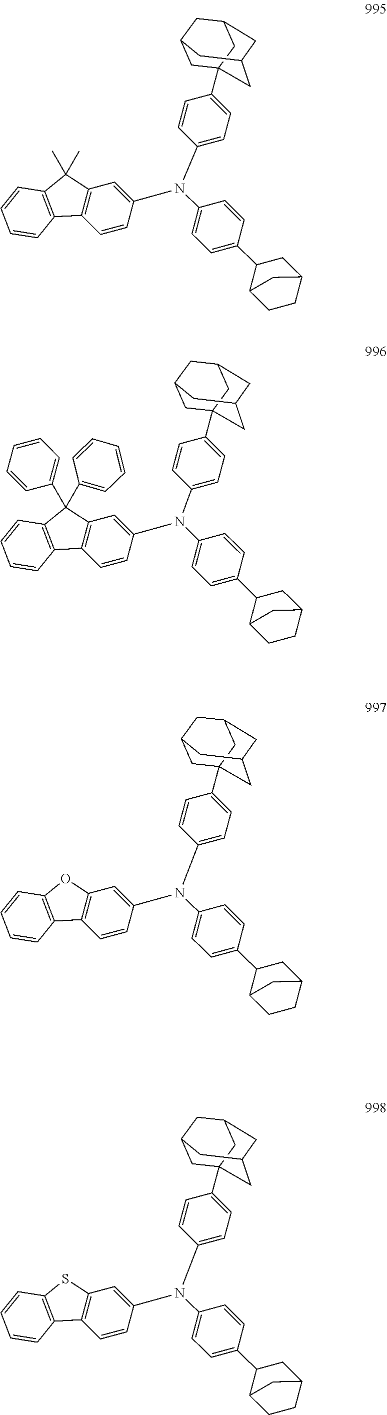 [SMILES: CC1(C)c2ccccc2-c2ccc(N(c3ccc(C4CC5CCC4C5)cc3)c3ccc(C45CC6CC(CC(C6)C4)C5)cc3)cc21 ; c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1 ; c1ccc2c(c1)oc1cc(N(c3ccc(C4CC5CCC4C5)cc3)c3ccc(C45CC6CC(CC(C6)C4)C5)cc3)ccc12 ; c1ccc2c(c1)sc1cc(N(c3ccc(C4CC5CCC4C5)cc3)c3ccc(C45CC6CC(CC(C6)C4)C5)cc3)ccc12]

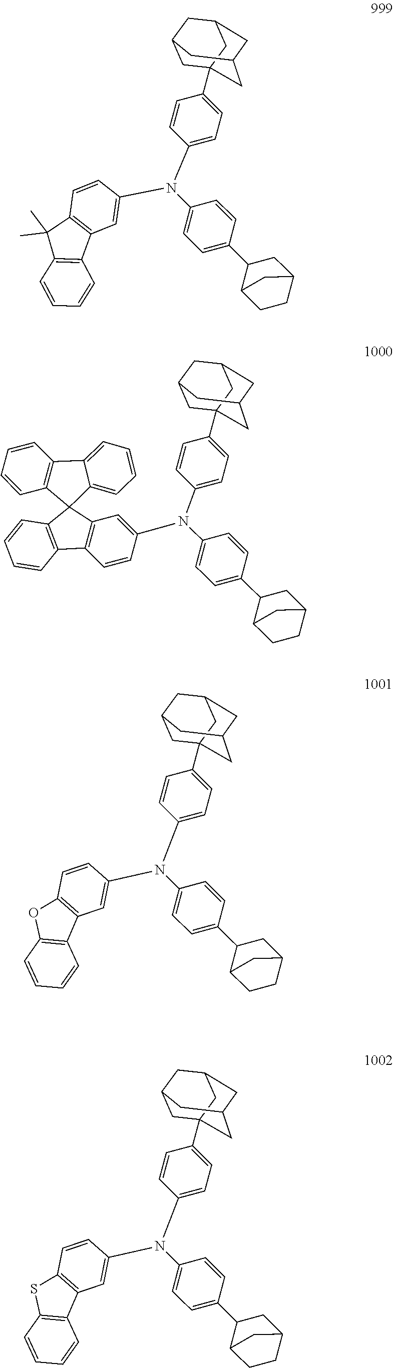 [SMILES: CC1(C)c2ccccc2-c2cc(N(c3ccc(C4CC5CCC4C5)cc3)c3ccc(C45CC6CC(CC(C6)C4)C5)cc3)ccc21 ; c1ccc2c(c1)-c1ccccc1C21c2ccccc2-c2ccc(N(c3ccc(C4CC5CCC4C5)cc3)c3ccc(C45CC6CC(CC(C6)C4)C5)cc3)cc21 ; c1ccc2c(c1)oc1ccc(N(c3ccc(C4CC5CCC4C5)cc3)c3ccc(C45CC6CC(CC(C6)C4)C5)cc3)cc12 ; c1ccc2c(c1)sc1ccc(N(c3ccc(C4CC5CCC4C5)cc3)c3ccc(C45CC6CC(CC(C6)C4)C5)cc3)cc12]

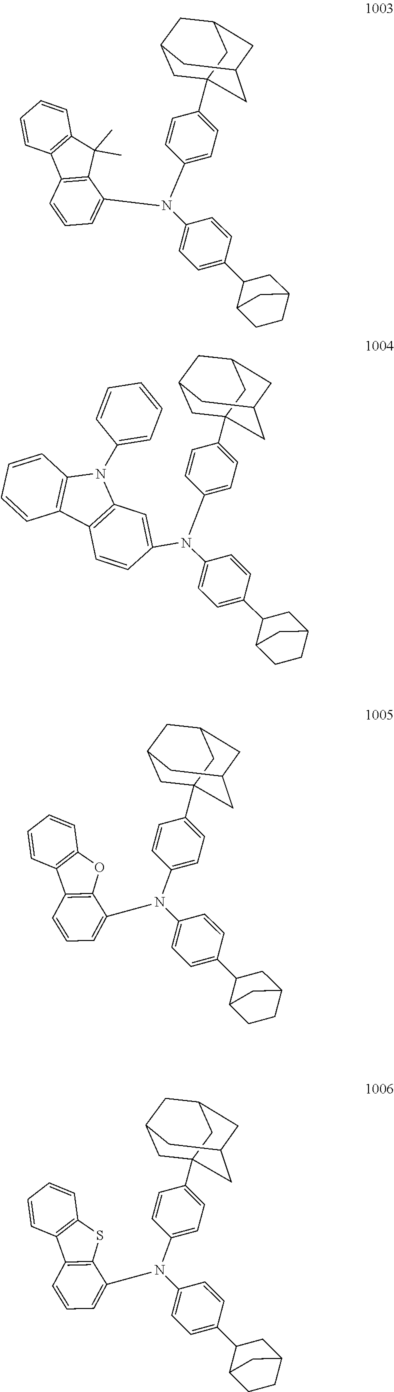 [SMILES: CC1(C)c2ccccc2-c2cccc(N(c3ccc(C4CC5CCC4C5)cc3)c3ccc(C45CC6CC(CC(C6)C4)C5)cc3)c21 ; c1ccc(-n2c3ccccc3c3ccc(N(c4ccc(C5CC6CCC5C6)cc4)c4ccc(C56CC7CC(CC(C7)C5)C6)cc4)cc32)cc1 ; c1ccc2c(c1)oc1c(N(c3ccc(C4CC5CCC4C5)cc3)c3ccc(C45CC6CC(CC(C6)C4)C5)cc3)cccc12 ; c1ccc2c(c1)sc1c(N(c3ccc(C4CC5CCC4C5)cc3)c3ccc(C45CC6CC(CC(C6)C4)C5)cc3)cccc12]

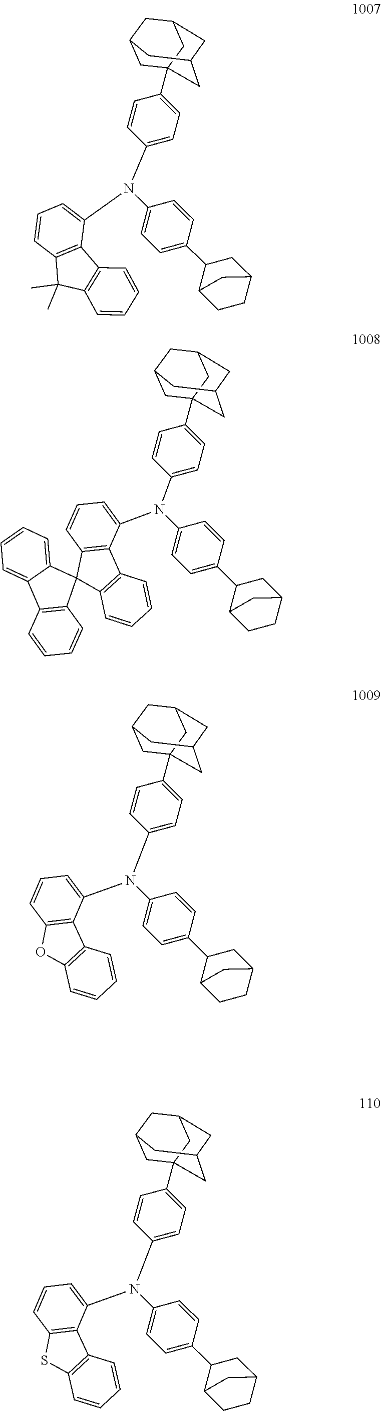 [SMILES: CC1(C)c2ccccc2-c2c(N(c3ccc(C4CC5CCC4C5)cc3)c3ccc(C45CC6CC(CC(C6)C4)C5)cc3)cccc21 ; c1ccc2c(c1)-c1ccccc1C21c2ccccc2-c2c(N(c3ccc(C4CC5CCC4C5)cc3)c3ccc(C45CC6CC(CC(C6)C4)C5)cc3)cccc21 ; c1ccc2c(c1)oc1cccc(N(c3ccc(C4CC5CCC4C5)cc3)c3ccc(C45CC6CC(CC(C6)C4)C5)cc3)c12 ; c1ccc2c(c1)sc1cccc(N(c3ccc(C4CC5CCC4C5)cc3)c3ccc(C45CC6CC(CC(C6)C4)C5)cc3)c12]

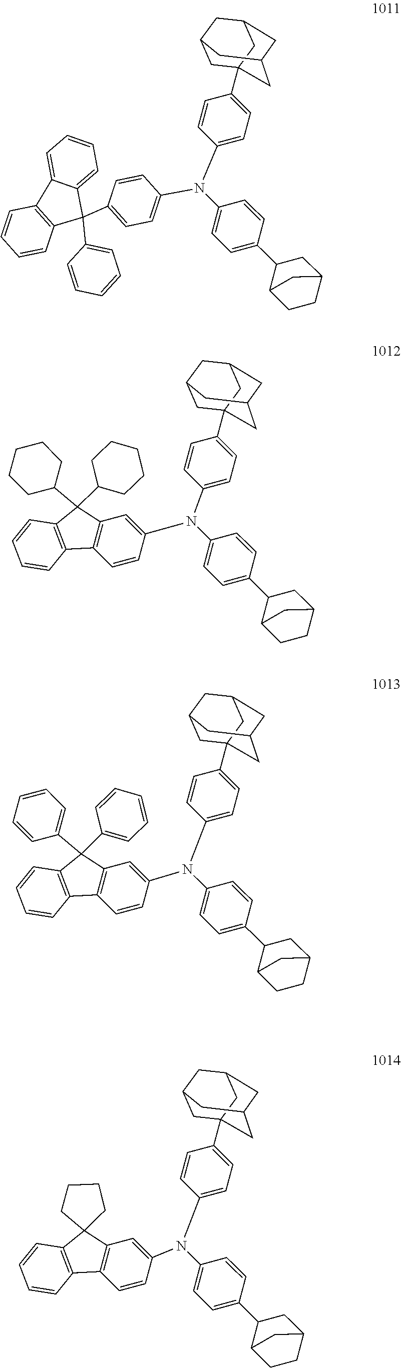 [SMILES: c1ccc(C2(c3ccc(N(c4ccc(C5CC6CCC5C6)cc4)c4ccc(C56CC7CC(CC(C7)C5)C6)cc4)cc3)c3ccccc3-c3ccccc32)cc1 ; c1ccc2c(c1)-c1ccc(N(c3ccc(C4CC5CCC4C5)cc3)c3ccc(C45CC6CC(CC(C6)C4)C5)cc3)cc1C2(C1CCCCC1)C1CCCCC1 ; c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1 ; c1ccc2c(c1)-c1ccc(N(c3ccc(C4CC5CCC4C5)cc3)c3ccc(C45CC6CC(CC(C6)C4)C5)cc3)cc1C21CCCC1]

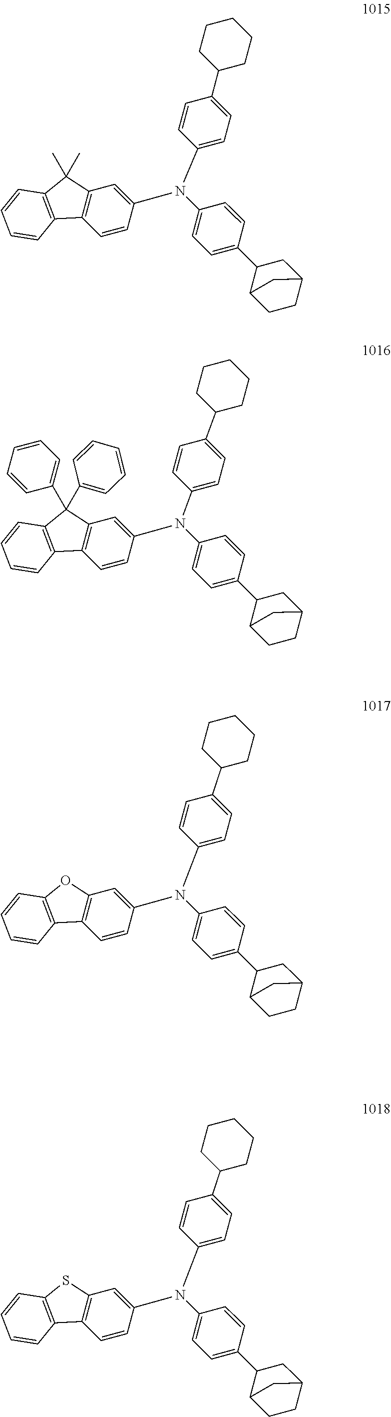 [SMILES: CC1(C)c2ccccc2-c2ccc(N(c3ccc(C4CCCCC4)cc3)c3ccc(C4CC5CCC4C5)cc3)cc21 ; c1ccc(C2(c3ccccc3)c3ccccc3-c3ccc(N(c4ccc(C5CCCCC5)cc4)c4ccc(C5CC6CCC5C6)cc4)cc32)cc1 ; c1ccc2c(c1)oc1cc(N(c3ccc(C4CCCCC4)cc3)c3ccc(C4CC5CCC4C5)cc3)ccc12 ; c1ccc2c(c1)sc1cc(N(c3ccc(C4CCCCC4)cc3)c3ccc(C4CC5CCC4C5)cc3)ccc12]

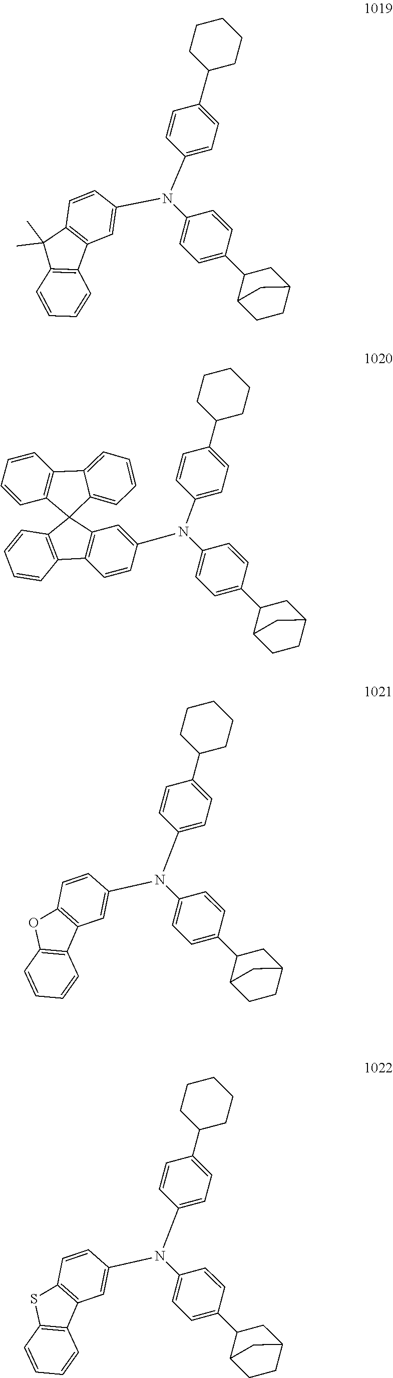 [SMILES: CC1(C)c2ccccc2-c2cc(N(c3ccc(C4CCCCC4)cc3)c3ccc(C4CC5CCC4C5)cc3)ccc21 ; c1ccc2c(c1)-c1ccccc1C21c2ccccc2-c2ccc(N(c3ccc(C4CCCCC4)cc3)c3ccc(C4CC5CCC4C5)cc3)cc21 ; c1ccc2c(c1)oc1ccc(N(c3ccc(C4CCCCC4)cc3)c3ccc(C4CC5CCC4C5)cc3)cc12 ; c1ccc2c(c1)sc1ccc(N(c3ccc(C4CCCCC4)cc3)c3ccc(C4CC5CCC4C5)cc3)cc12]

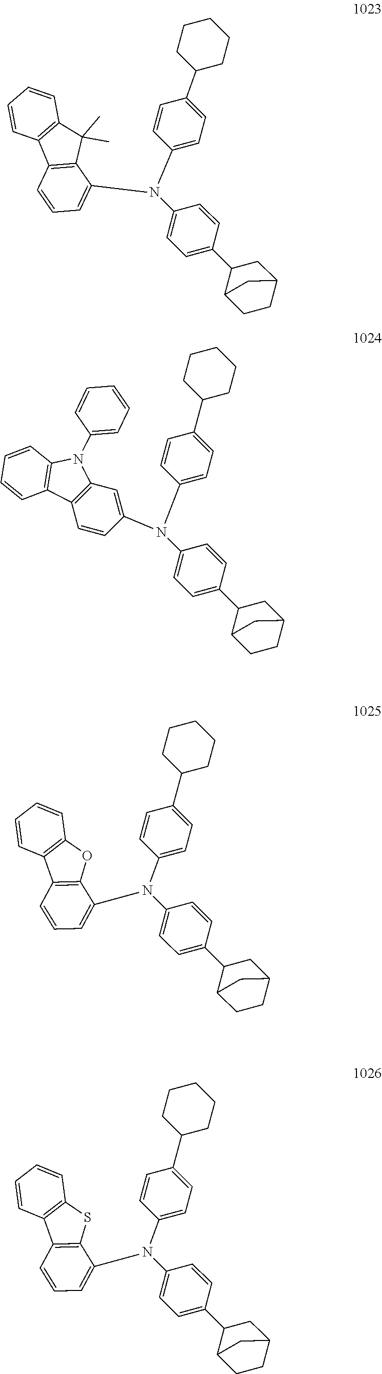 [SMILES: CC1(C)c2ccccc2-c2cccc(N(c3ccc(C4CCCCC4)cc3)c3ccc(C4CC5CCC4C5)cc3)c21 ; c1ccc(-n2c3ccccc3c3ccc(N(c4ccc(C5CCCCC5)cc4)c4ccc(C5CC6CCC5C6)cc4)cc32)cc1 ; c1ccc2c(c1)oc1c(N(c3ccc(C4CCCCC4)cc3)c3ccc(C4CC5CCC4C5)cc3)cccc12 ; c1ccc2c(c1)sc1c(N(c3ccc(C4CCCCC4)cc3)c3ccc(C4CC5CCC4C5)cc3)cccc12]

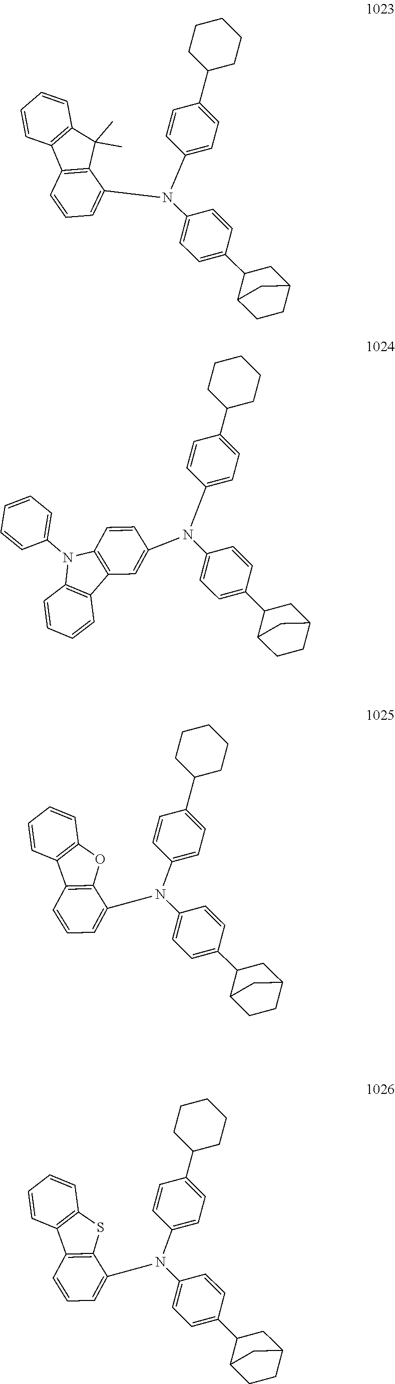 [SMILES: CC1(C)c2ccccc2-c2cccc(N(c3ccc(C4CCCCC4)cc3)c3ccc(C4CC5CCC4C5)cc3)c21 ; c1ccc(-n2c3ccccc3c3cc(N(c4ccc(C5CCCCC5)cc4)c4ccc(C5CC6CCC5C6)cc4)ccc32)cc1 ; c1ccc2c(c1)oc1c(N(c3ccc(C4CCCCC4)cc3)c3ccc(C4CC5CCC4C5)cc3)cccc12 ; c1ccc2c(c1)sc1c(N(c3ccc(C4CCCCC4)cc3)c3ccc(C4CC5CCC4C5)cc3)cccc12]

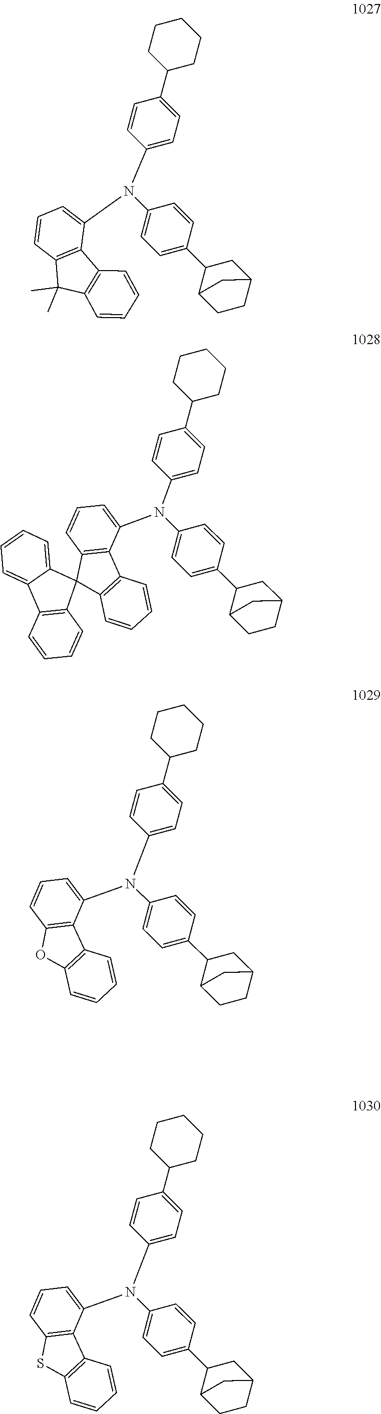 [SMILES: CC1(C)c2ccccc2-c2c(N(c3ccc(C4CCCCC4)cc3)c3ccc(C4CC5CCC4C5)cc3)cccc21 ; c1ccc2c(c1)-c1ccccc1C21c2ccccc2-c2c(N(c3ccc(C4CCCCC4)cc3)c3ccc(C4CC5CCC4C5)cc3)cccc21 ; c1ccc2c(c1)oc1cccc(N(c3ccc(C4CCCCC4)cc3)c3ccc(C4CC5CCC4C5)cc3)c12 ; c1ccc2c(c1)sc1cccc(N(c3ccc(C4CCCCC4)cc3)c3ccc(C4CC5CCC4C5)cc3)c12]

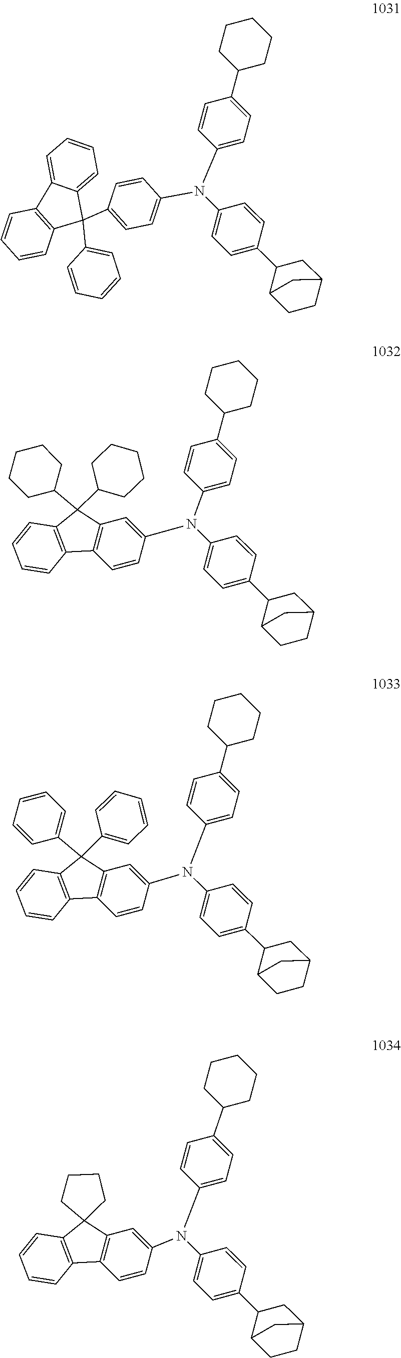 [SMILES: c1ccc(C2(c3ccc(N(c4ccc(C5CCCCC5)cc4)c4ccc(C5CC6CCC5C6)cc4)cc3)c3ccccc3-c3ccccc32)cc1 ; c1ccc2c(c1)-c1ccc(N(c3ccc(C4CCCCC4)cc3)c3ccc(C4CC5CCC4C5)cc3)cc1C2(C1CCCCC1)C1CCCCC1 ; c1ccc(C2(c3ccccc3)c3ccccc3-c3ccc(N(c4ccc(C5CCCCC5)cc4)c4ccc(C5CC6CCC5C6)cc4)cc32)cc1 ; c1ccc2c(c1)-c1ccc(N(c3ccc(C4CCCCC4)cc3)c3ccc(C4CC5CCC4C5)cc3)cc1C21CCCC1]

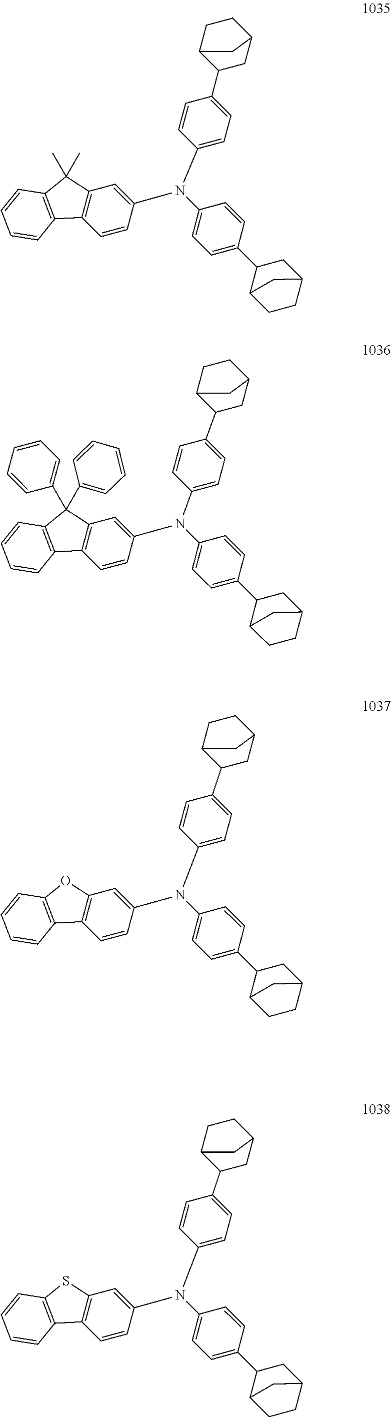 [SMILES: CC1(C)c2ccccc2-c2ccc(N(c3ccc(C4CC5CCC4C5)cc3)c3ccc(C4CC5CCC4C5)cc3)cc21 ; c1ccc(C2(c3ccccc3)c3ccccc3-c3ccc(N(c4ccc(C5CC6CCC5C6)cc4)c4ccc(C5CC6CCC5C6)cc4)cc32)cc1 ; c1ccc2c(c1)oc1cc(N(c3ccc(C4CC5CCC4C5)cc3)c3ccc(C4CC5CCC4C5)cc3)ccc12 ; c1ccc2c(c1)sc1cc(N(c3ccc(C4CC5CCC4C5)cc3)c3ccc(C4CC5CCC4C5)cc3)ccc12]

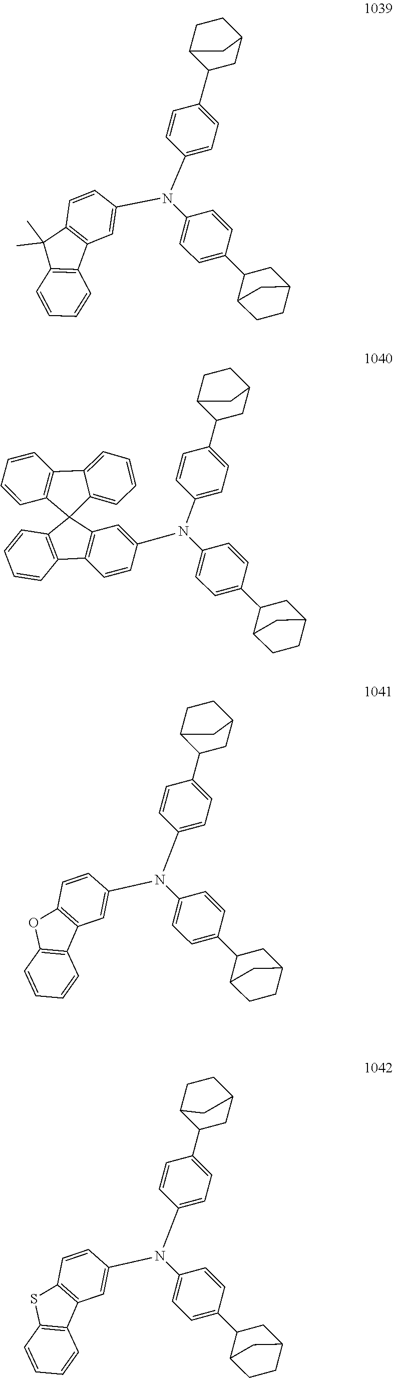 [SMILES: CC1(C)c2ccccc2-c2cc(N(c3ccc(C4CC5CCC4C5)cc3)c3ccc(C4CC5CCC4C5)cc3)ccc21 ; c1ccc2c(c1)-c1ccccc1C21c2ccccc2-c2ccc(N(c3ccc(C4CC5CCC4C5)cc3)c3ccc(C4CC5CCC4C5)cc3)cc21 ; c1ccc2c(c1)oc1ccc(N(c3ccc(C4CC5CCC4C5)cc3)c3ccc(C4CC5CCC4C5)cc3)cc12 ; c1ccc2c(c1)sc1ccc(N(c3ccc(C4CC5CCC4C5)cc3)c3ccc(C4CC5CCC4C5)cc3)cc12]

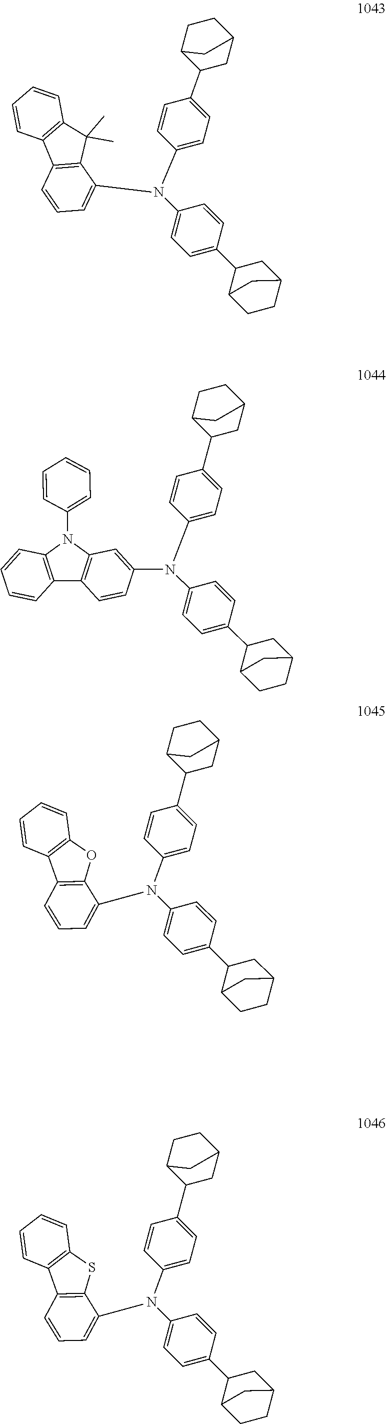 [SMILES: CC1(C)c2ccccc2-c2cccc(N(c3ccc(C4CC5CCC4C5)cc3)c3ccc(C4CC5CCC4C5)cc3)c21 ; c1ccc(-n2c3ccccc3c3ccc(N(c4ccc(C5CC6CCC5C6)cc4)c4ccc(C5CC6CCC5C6)cc4)cc32)cc1 ; c1ccc2c(c1)oc1c(N(c3ccc(C4CC5CCC4C5)cc3)c3ccc(C4CC5CCC4C5)cc3)cccc12 ; c1ccc2c(c1)sc1c(N(c3ccc(C4CC5CCC4C5)cc3)c3ccc(C4CC5CCC4C5)cc3)cccc12]

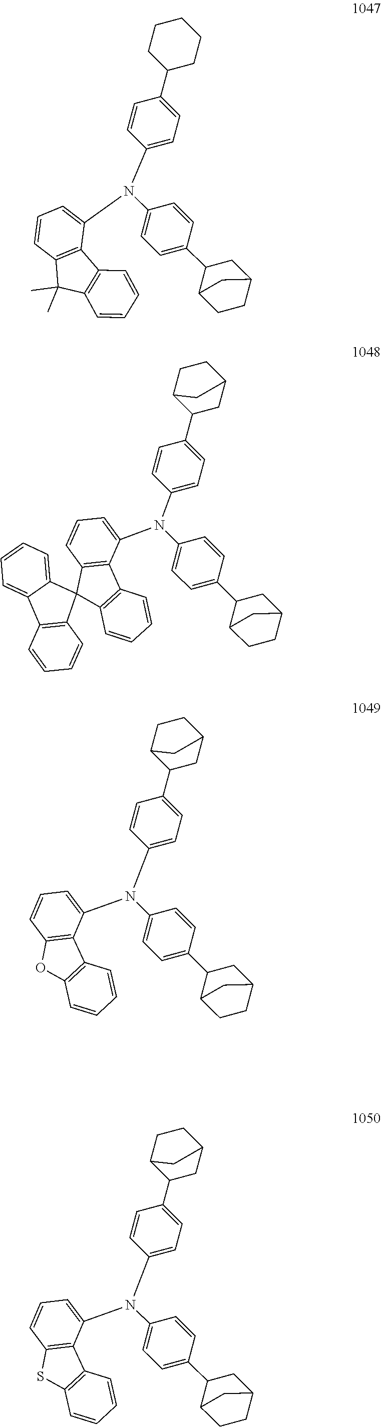 [SMILES: CC1(C)c2ccccc2-c2c(N(c3ccc(C4CCCCC4)cc3)c3ccc(C4CC5CCC4C5)cc3)cccc21 ; c1ccc2c(c1)-c1ccccc1C21c2ccccc2-c2c(N(c3ccc(C4CC5CCC4C5)cc3)c3ccc(C4CC5CCC4C5)cc3)cccc21 ; c1ccc2c(c1)oc1cccc(N(c3ccc(C4CC5CCC4C5)cc3)c3ccc(C4CC5CCC4C5)cc3)c12 ; c1ccc2c(c1)sc1cccc(N(c3ccc(C4CC5CCC4C5)cc3)c3ccc(C4CC5CCC4C5)cc3)c12]

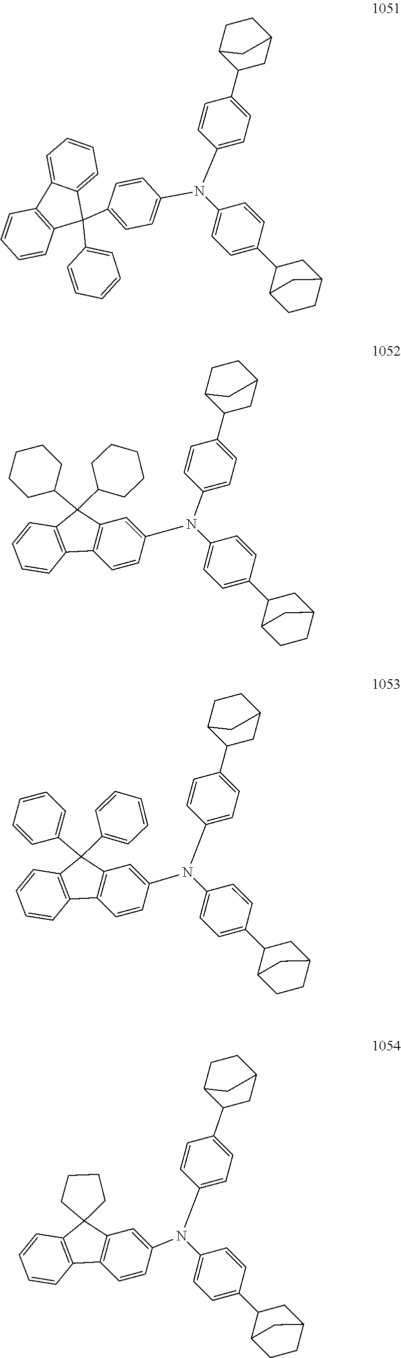 [SMILES: c1ccc(C2(c3ccc(N(c4ccc(C5CC6CCC5C6)cc4)c4ccc(C5CC6CCC5C6)cc4)cc3)c3ccccc3-c3ccccc32)cc1 ; c1ccc2c(c1)-c1ccc(N(c3ccc(C4CC5CCC4C5)cc3)c3ccc(C4CC5CCC4C5)cc3)cc1C2(C1CCCCC1)C1CCCCC1 ; c1ccc(C2(c3ccccc3)c3ccccc3-c3ccc(N(c4ccc(C5CC6CCC5C6)cc4)c4ccc(C5CC6CCC5C6)cc4)cc32)cc1 ; c1ccc2c(c1)-c1ccc(N(c3ccc(C4CC5CCC4C5)cc3)c3ccc(C4CC5CCC4C5)cc3)cc1C21CCCC1]

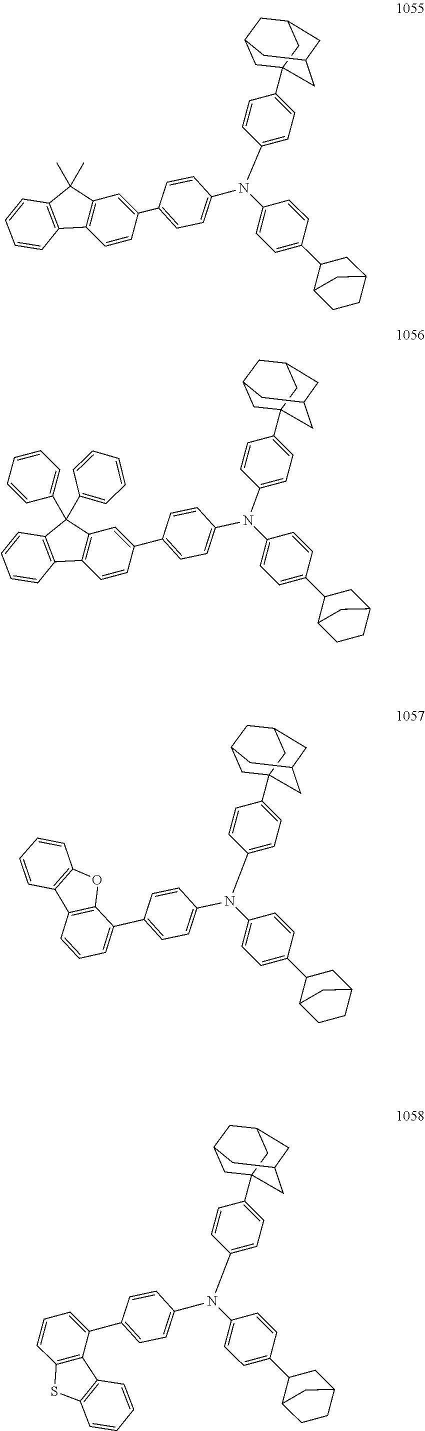 [SMILES: CC1(C)c2ccccc2-c2ccc(-c3ccc(N(c4ccc(C5CC6CCC5C6)cc4)c4ccc(C56CC7CC(CC(C7)C5)C6)cc4)cc3)cc21 ; c1ccc(C2(c3ccccc3)c3ccccc3-c3ccc(-c4ccc(N(c5ccc(C6CC7CCC6C7)cc5)c5ccc(C67CC8CC(CC(C8)C6)C7)cc5)cc4)cc32)cc1 ; c1ccc2c(c1)oc1c(-c3ccc(N(c4ccc(C5CC6CCC5C6)cc4)c4ccc(C56CC7CC(CC(C7)C5)C6)cc4)cc3)cccc12 ; c1ccc2c(c1)sc1cccc(-c3ccc(N(c4ccc(C5CC6CCC5C6)cc4)c4ccc(C56CC7CC(CC(C7)C5)C6)cc4)cc3)c12]

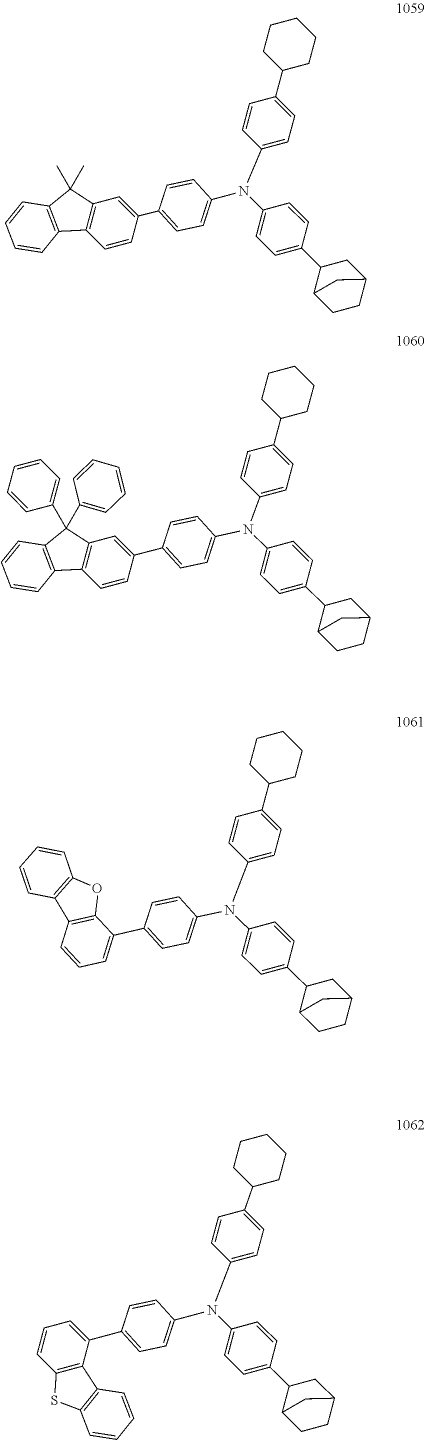 [SMILES: CC1(C)c2ccccc2-c2ccc(-c3ccc(N(c4ccc(C5CCCCC5)cc4)c4ccc(C5CC6CCC5C6)cc4)cc3)cc21 ; c1ccc(C2(c3ccccc3)c3ccccc3-c3ccc(-c4ccc(N(c5ccc(C6CCCCC6)cc5)c5ccc(C6CC7CCC6C7)cc5)cc4)cc32)cc1 ; c1ccc2c(c1)oc1c(-c3ccc(N(c4ccc(C5CCCCC5)cc4)c4ccc(C5CC6CCC5C6)cc4)cc3)cccc12 ; c1ccc2c(c1)sc1cccc(-c3ccc(N(c4ccc(C5CCCCC5)cc4)c4ccc(C5CC6CCC5C6)cc4)cc3)c12]

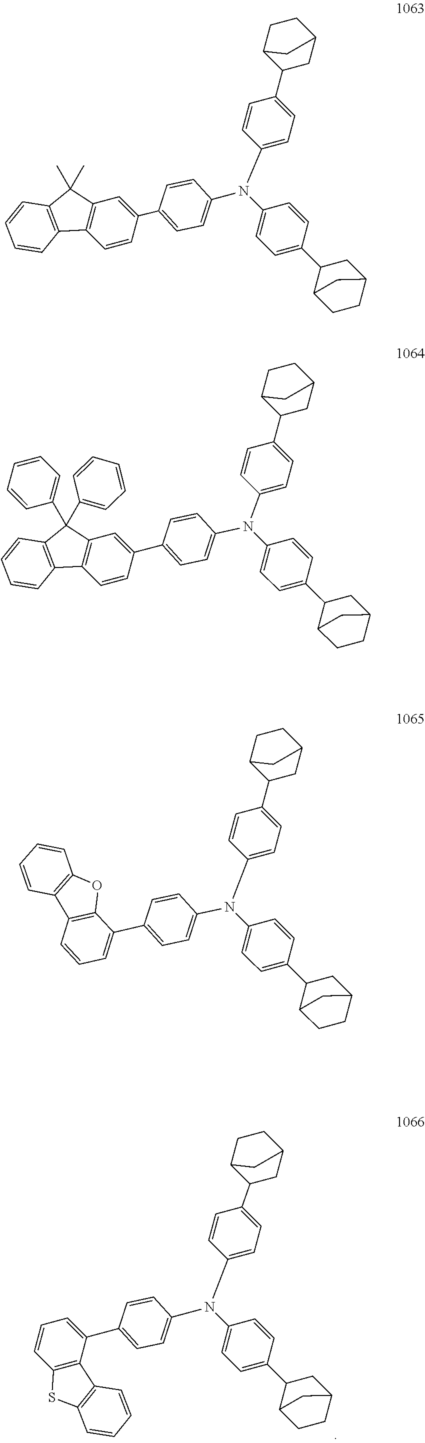 [SMILES: CC1(C)c2ccccc2-c2ccc(-c3ccc(N(c4ccc(C5CC6CCC5C6)cc4)c4ccc(C5CC6CCC5C6)cc4)cc3)cc21 ; c1ccc(C2(c3ccccc3)c3ccccc3-c3ccc(-c4ccc(N(c5ccc(C6CC7CCC6C7)cc5)c5ccc(C6CC7CCC6C7)cc5)cc4)cc32)cc1 ; c1ccc2c(c1)oc1c(-c3ccc(N(c4ccc(C5CC6CCC5C6)cc4)c4ccc(C5CC6CCC5C6)cc4)cc3)cccc12 ; c1ccc2c(c1)sc1cccc(-c3ccc(N(c4ccc(C5CC6CCC5C6)cc4)c4ccc(C5CC6CCC5C6)cc4)cc3)c12]

The amine compound according to an embodiment essentially includes one of the first fused ring and has a structure essentially including a first substituent and a second substituent in a molecular structure. The first substituent and the second substituent may be each independently one substituent selected from an adamantyl group, a cyclohexyl group, a bicycloheptanyl group, a bicyclooctanyl group, and a triphenylsilyl group and may be combined with the nitrogen atom of an amine group. The amine compound according to an embodiment may include a first fused ring, a first substituent, and a second substituent, combined with an amine group. Each of the first fused ring, the first substituent, and the second substituent may be combined with the nitrogen atom of the amine group via a first to third connecting groups.

The amine compound according to an embodiment has a structure essentially including the first substituent and the second substituent selected from an adamantyl group, a cyclohexyl group, a bicycloheptanyl group, a bicyclooctanyl group, and a triphenylsilyl group, and may reduce intermolecular interaction and have low packing density. Accordingly, the amine compound of an embodiment has a low refractive index and may change the refractive index of a molecule by diversely changing the combination of the first substituent and the second substituent. In addition, the amine compound according to an embodiment includes the first substituent and the second substituent, and the deposition temperature may be reduced during forming a layer by a deposition process to improve thin layer stability and process productivity during forming the layer.

In addition, the amine compound of an embodiment may include a third substituent which is substituted at the first fused ring, or include a structure in which at least one selected from among the first substituent and the second substituent, combined with the amine group is a substituted or unsubstituted bicycloheptanyl group. For example, in the amine compound according to an embodiment, the first fused ring may be directly connected with the nitrogen atom of the amine group, and may include at least one or more third substituents as the substituent connected with the first fused ring in addition to the amine group. The third substituent may be a substituted or unsubstituted aryl group, or a substituted or unsubstituted heteroaryl group. Otherwise, the amine compound of an embodiment may essentially include a substituted or unsubstituted bicycloheptanyl group as a substituent combined with the amine group. In some embodiments, at least one selected from among the first substituent and the second substituent, combined with the amine compound of an embodiment may be a substituted or unsubstituted bicycloheptanyl group.

The amine compound according to an embodiment may diversely change the highest occupied molecular orbital (HOMO) energy level by changing the type and connecting positions of the first substituent and the second substituent, substituted at the amine group, the position of substitution of the third substituent at the first fused ring, and the number of the third substituents. Accordingly, the hole injection barrier between the first electrode EL1 and the hole transport region HTR may be changed diversely, to achieve a suitable energy level between the hole transport region HTR and the emission layer EML, so as to control to increase the excitons-production efficiency in the emission layer EML. Accordingly, if the amine compound according to an embodiment of the present disclosure is applied to the hole transport region HTR of the light emitting device ED, a light emitting device having high efficiency, a low voltage, high luminance, and long life may be achieved.

If the amine compound of an embodiment is used in the hole transport region, external quantum efficiency may be increased by changing the refractive index change and light extraction mode between the first electrode and the second electrode. Accordingly, if the amine compound of an embodiment is used in the hole transport region, the emission efficiency of the light emitting device may be increased, and the life of the light emitting device may be improved. In addition, with excellent heat resistance and durability as described above, by including the amine compound of an embodiment as the material of the light emitting device, the life and emission efficiency of the light emitting device of an embodiment may be improved.

In some embodiments, if the light emitting device ED of an embodiment includes multiple hole transport layers HTL1, HTL2 and HTL3, each of the first hole transport layer HTL1 adjacent to the first electrode EU and the third hole transport layer HTL3 adjacent to the emission layer EML may include the amine compound of an embodiment, represented by Formula 1. In addition, the second hole transport layer HTL2 between the first hole transport layer HTL1 and the third hole transport layer HTL3 may include an amine derivative compound represented by Formula 10 below.

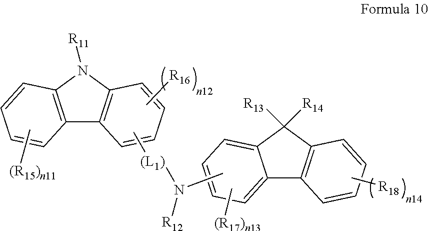

In Formula 10, L₁ may be a direct linkage, a substituted or unsubstituted arylene group of 6 to 30 ring-forming carbon atoms, or a substituted or unsubstituted heteroarylene group of 2 to 30 ring-forming carbon atoms.

In Formula 10, R₁₁ to R₁₄ may be each independently a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms. Otherwise, each of R₁₁ to R₁₄ may be combined with an adjacent group to form a ring. For example, R₁₁ to R₁₄ may be each independently a substituted or unsubstituted methyl group, or a substituted or unsubstituted phenyl group.

In Formula 10, R₁₅ to R₁₈ may be each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, a substituted or unsubstituted heteroaryl group of 2 to 30 ring-forming carbon atoms. Otherwise, R₁₅ to R₁₈ may be combined with adjacent groups to form rings. For example, R₁₅ to R₁₈ may be each independently a hydrogen atom.

In Formula 10, n11 and n14 are each independently an integer of 0 to 4, and n12 and n13 are each independently an integer of 0 to 3.

If n11 and n14 are 0, the amine compound of an embodiment may be unsubstituted with R₁₅ and R₁₈, respectively. The case where n11 and n14 are 4, and R₁₅ and R₁₈ are all hydrogen atoms in Formula 10, may be the same as the case where n11 and n14 are 0 in Formula 10. If n11 and n14 are integers of 2 or more, each of multiple R₁₅ and R₁₈ may be the same, or at least one of multiple R₁₅ and R₁₈ may be different.

If n12 and n13 are 0, the amine compound of an embodiment may be unsubstituted with R₁₆ and R₁₇, respectively. The case where n12 and n13 are 3, and R₁₆ and R₁₇ are all hydrogen atoms in Formula 10, may be the same as the case where n12 and n13 are 0 in Formula 10. If n12 and n13 are integers of 2 or more, each of multiple R₁₆ and R₁₇ may be the same, or at least one of multiple R₁₆ and R₁₇ may be different.

In the amine derivative represented by Formula 10, R₁₂ may be an aryl group or a heteroaryl group. For example, R₁₂ may be a substituted or unsubstituted phenyl group, a substituted or unsubstituted naphthyl group, a substituted or unsubstituted biphenyl group, a substituted or unsubstituted dibenzofuran group, or a substituted or unsubstituted dibenzothiophene group.

In an embodiment, the amine derivative compound represented by Formula 10 may be represented by Formula 11-1 or Formula 11-2 below.

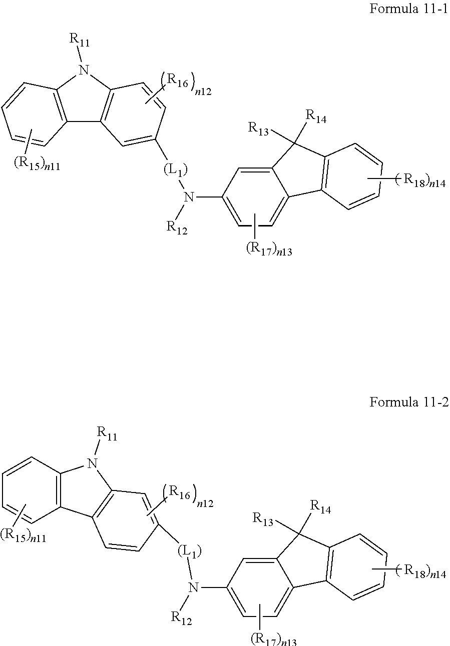

Formula 11-1 and Formula 11-2 represent cases of Formula 10 where the position connected of a carbazole moiety and L₁ is specified. Formula 11-1 corresponds to a case of Formula 10 where the carbon position 3 of the carbazole moiety is the position connected with L₁. Formula 11-2 corresponds to a case of Formula 10 where the carbon position 2 of the carbazole moiety is the position connected with L₁.

In Formula 11-1 and Formula 11-2, the same description of L₁, R₁₁ to R₁₄, R₁₅ to R₁₈, and n11 to n13 referring to Formula 10 may be applied.

The amine derivative compound represented by Formula 10 may be represented by any one selected from among the compounds represented in Compound Group 2 below. For example, the second hole transport layer HTL2 may include at least one selected from among the compounds represented in Compound Group 2 below.

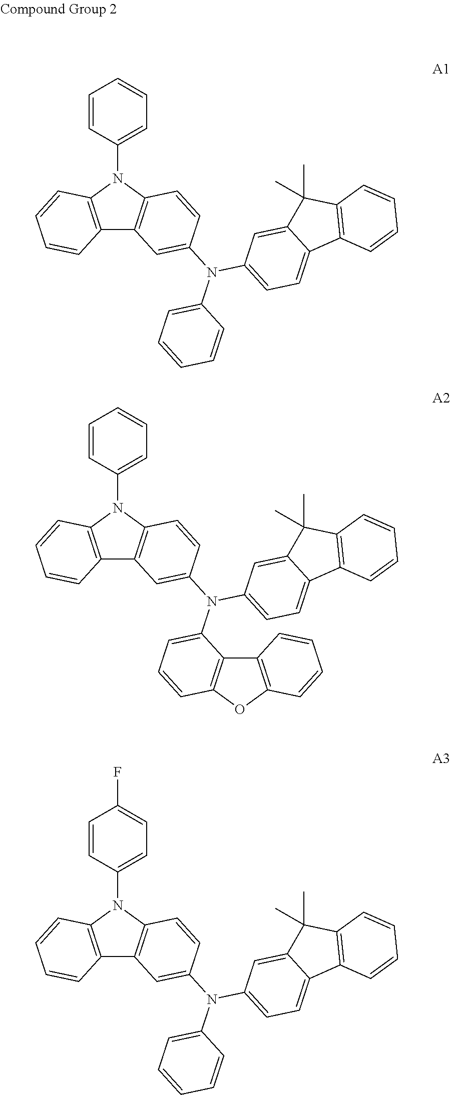 [SMILES: CC1(C)c2ccccc2-c2ccc(N(c3ccccc3)c3ccc4c(c3)c3ccccc3n4-c3ccccc3)cc21 ; CC1(C)c2ccccc2-c2ccc(N(c3ccc4c(c3)c3ccccc3n4-c3ccccc3)c3cccc4oc5ccccc5c34)cc21 ; CC1(C)c2ccccc2-c2ccc(N(c3ccccc3)c3ccc4c(c3)c3ccccc3n4-c3ccc(F)cc3)cc21]

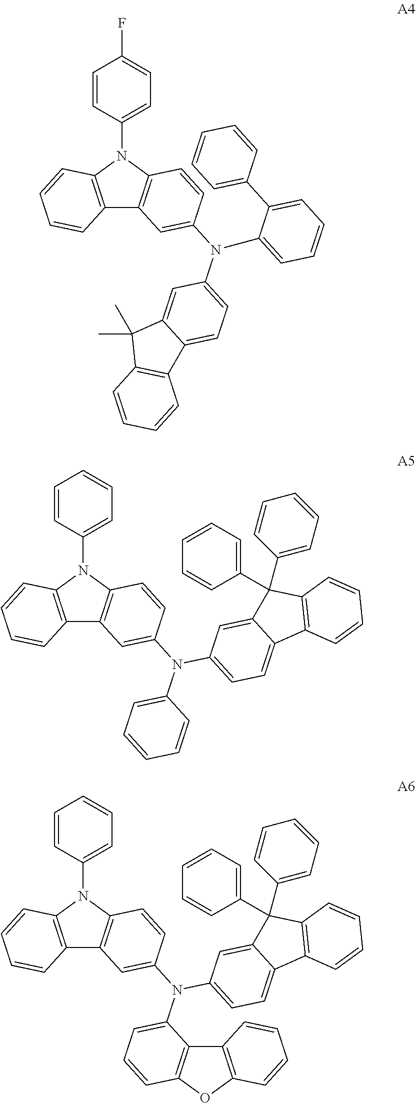 [SMILES: CC1(C)c2ccccc2-c2ccc(N(c3ccc4c(c3)c3ccccc3n4-c3ccc(F)cc3)c3ccccc3-c3ccccc3)cc21 ; c1ccc(N(c2ccc3c(c2)C(c2ccccc2)(c2ccccc2)c2ccccc2-3)c2ccc3c(c2)c2ccccc2n3-c2ccccc2)cc1 ; c1ccc(-n2c3ccccc3c3cc(N(c4ccc5c(c4)C(c4ccccc4)(c4ccccc4)c4ccccc4-5)c4cccc5oc6ccccc6c45)ccc32)cc1]

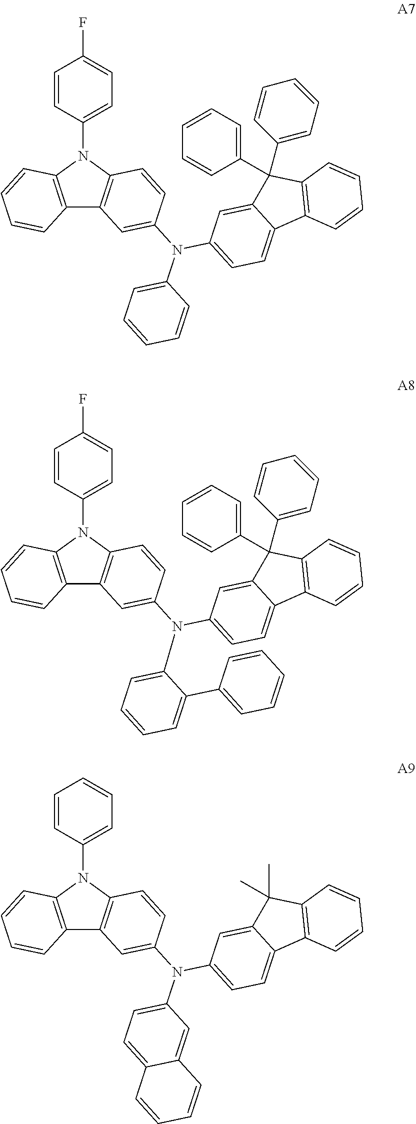 [SMILES: Fc1ccc(-n2c3ccccc3c3cc(N(c4ccccc4)c4ccc5c(c4)C(c4ccccc4)(c4ccccc4)c4ccccc4-5)ccc32)cc1 ; Fc1ccc(-n2c3ccccc3c3cc(N(c4ccc5c(c4)C(c4ccccc4)(c4ccccc4)c4ccccc4-5)c4ccccc4-c4ccccc4)ccc32)cc1 ; CC1(C)c2ccccc2-c2ccc(N(c3ccc4ccccc4c3)c3ccc4c(c3)c3ccccc3n4-c3ccccc3)cc21]

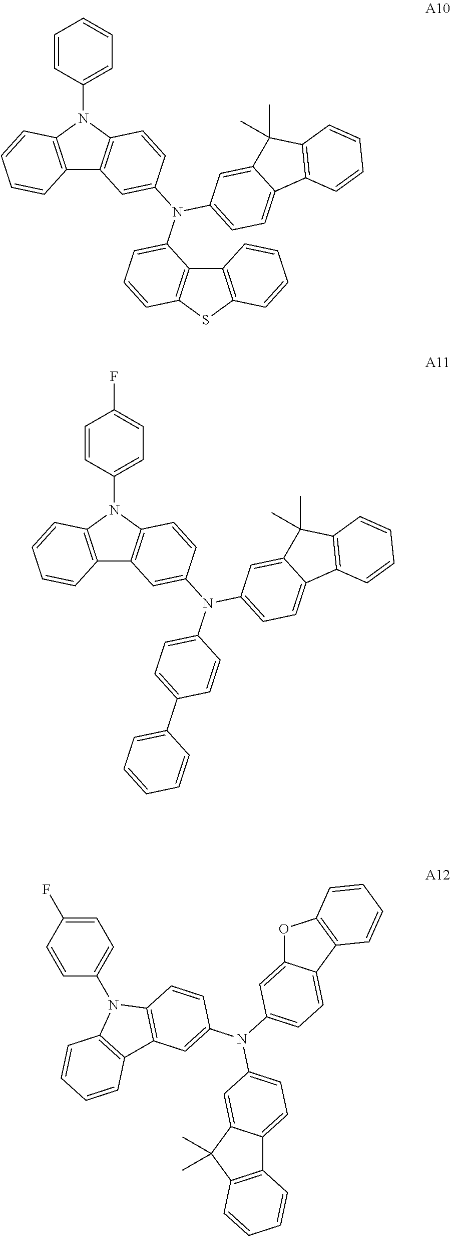 [SMILES: CC1(C)c2ccccc2-c2ccc(N(c3ccc4c(c3)c3ccccc3n4-c3ccccc3)c3cccc4sc5ccccc5c34)cc21 ; CC1(C)c2ccccc2-c2ccc(N(c3ccc(-c4ccccc4)cc3)c3ccc4c(c3)c3ccccc3n4-c3ccc(F)cc3)cc21 ; CC1(C)c2ccccc2-c2ccc(N(c3ccc4c(c3)oc3ccccc34)c3ccc4c(c3)c3ccccc3n4-c3ccc(F)cc3)cc21]

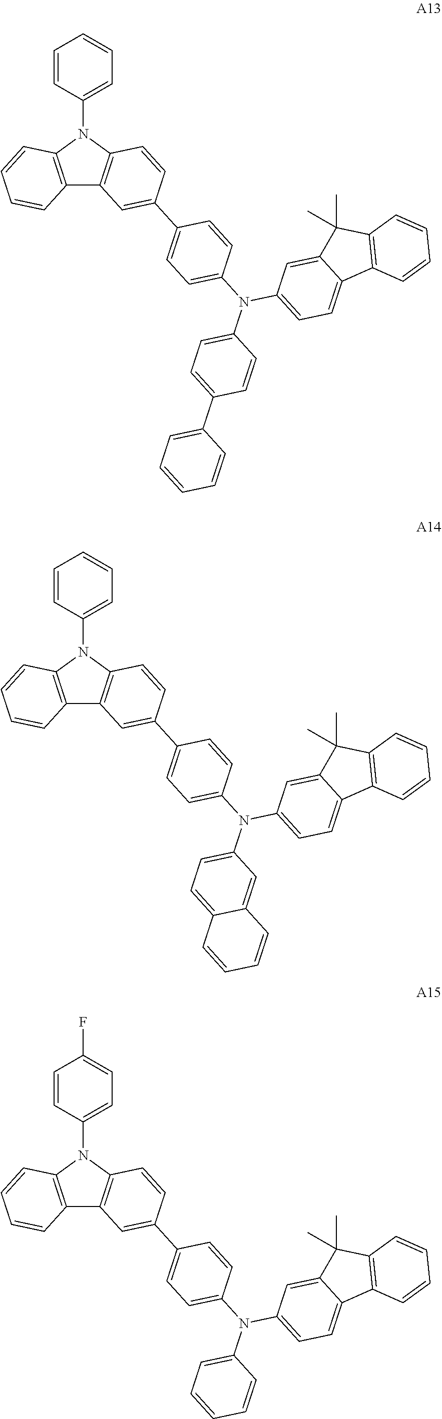 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3ccc4ccccc4c3)cc21 ; CC1(C)c2ccccc2-c2ccc(N(c3ccccc3)c3ccc(-c4ccc5c(c4)c4ccccc4n5-c4ccc(F)cc4)cc3)cc21]

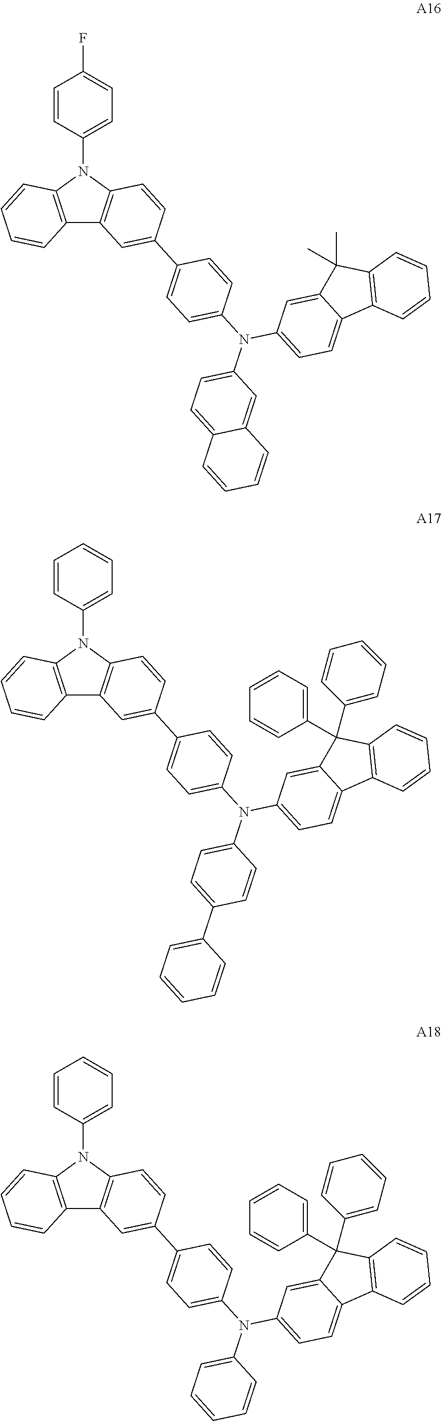 [SMILES: CC1(C)c2ccccc2-c2ccc(N(c3ccc(-c4ccc5c(c4)c4ccccc4n5-c4ccc(F)cc4)cc3)c3ccc4ccccc4c3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

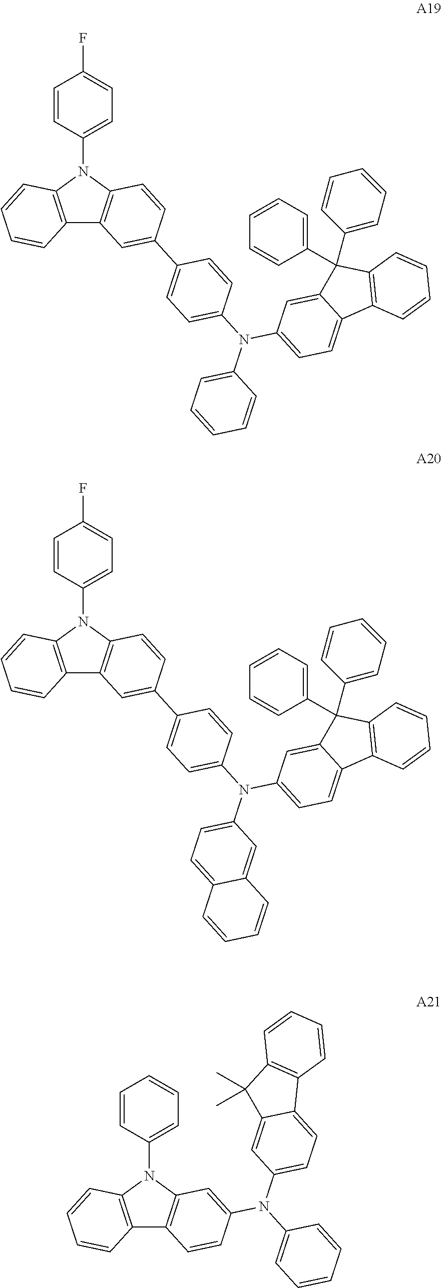 [SMILES: Fc1ccc(-n2c3ccccc3c3cc(-c4ccc(N(c5ccccc5)c5ccc6c(c5)C(c5ccccc5)(c5ccccc5)c5ccccc5-6)cc4)ccc32)cc1 ; Fc1ccc(-n2c3ccccc3c3cc(-c4ccc(N(c5ccc6c(c5)C(c5ccccc5)(c5ccccc5)c5ccccc5-6)c5ccc6ccccc6c5)cc4)ccc32)cc1 ; CC1(C)c2ccccc2-c2ccc(N(c3ccccc3)c3ccc4c5ccccc5n(-c5ccccc5)c4c3)cc21]

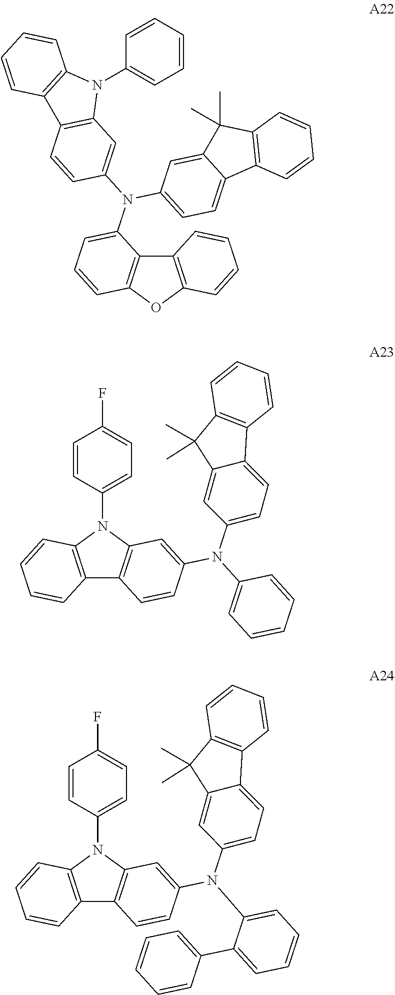 [SMILES: CC1(C)c2ccccc2-c2ccc(N(c3ccc4c5ccccc5n(-c5ccccc5)c4c3)c3cccc4oc5ccccc5c34)cc21 ; CC1(C)c2ccccc2-c2ccc(N(c3ccccc3)c3ccc4c5ccccc5n(-c5ccc(F)cc5)c4c3)cc21 ; CC1(C)c2ccccc2-c2ccc(N(c3ccc4c5ccccc5n(-c5ccc(F)cc5)c4c3)c3ccccc3-c3ccccc3)cc21]

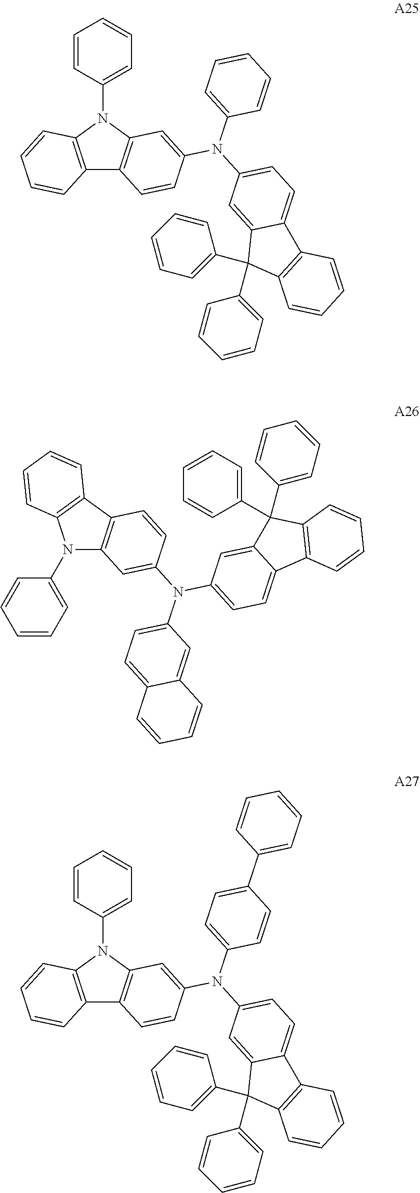 [SMILES: c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

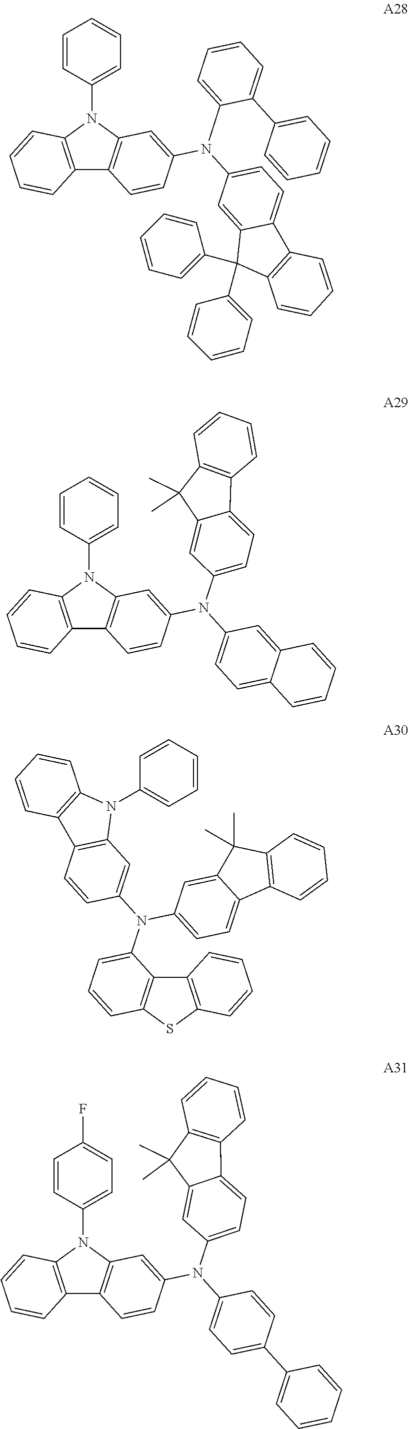 [SMILES: c1ccc(-c2ccccc2N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2ccc(N(c3ccc4ccccc4c3)c3ccc4c5ccccc5n(-c5ccccc5)c4c3)cc21 ; CC1(C)c2ccccc2-c2ccc(N(c3ccc4c5ccccc5n(-c5ccccc5)c4c3)c3cccc4sc5ccccc5c34)cc21 ; CC1(C)c2ccccc2-c2ccc(N(c3ccc(-c4ccccc4)cc3)c3ccc4c5ccccc5n(-c5ccc(F)cc5)c4c3)cc21]

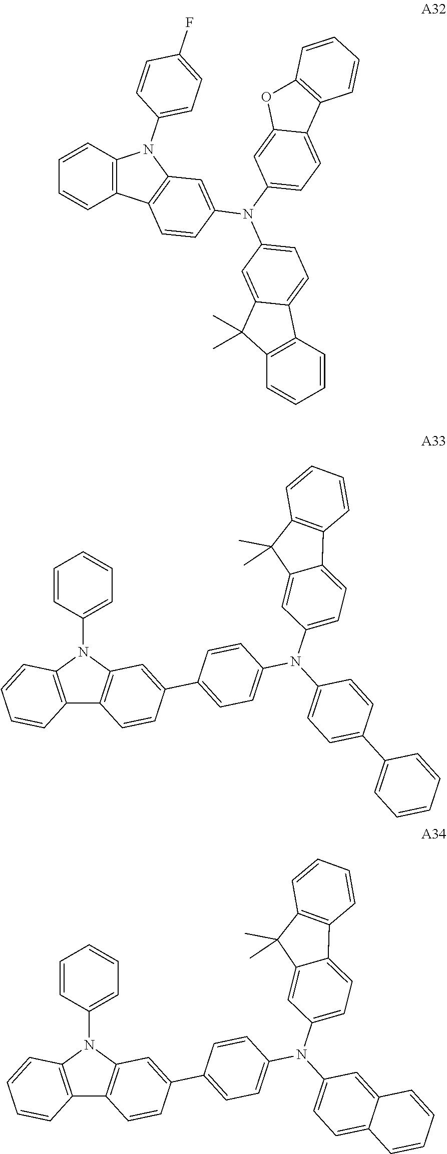 [SMILES: CC1(C)c2ccccc2-c2ccc(N(c3ccc4c(c3)oc3ccccc34)c3ccc4c5ccccc5n(-c5ccc(F)cc5)c4c3)cc21 ; CC1(C)c2ccccc2-c2ccc(N(c3ccc(-c4ccccc4)cc3)c3ccc(-c4ccc5c6ccccc6n(-c6ccccc6)c5c4)cc3)cc21 ; CC1(C)c2ccccc2-c2ccc(N(c3ccc(-c4ccc5c6ccccc6n(-c6ccccc6)c5c4)cc3)c3ccc4ccccc4c3)cc21]

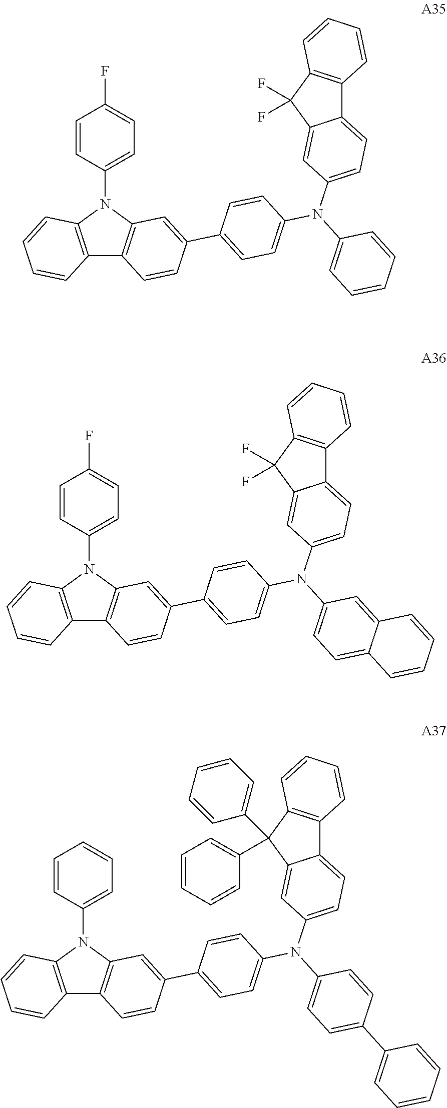 [SMILES: Fc1ccc(-n2c3ccccc3c3ccc(-c4ccc(N(c5ccccc5)c5ccc6c(c5)C(F)(F)c5ccccc5-6)cc4)cc32)cc1 ; Fc1ccc(-n2c3ccccc3c3ccc(-c4ccc(N(c5ccc6c(c5)C(F)(F)c5ccccc5-6)c5ccc6ccccc6c5)cc4)cc32)cc1 ; c1ccc(-c2ccc(N(c3ccc(-c4ccc5c6ccccc6n(-c6ccccc6)c5c4)cc3)c3ccc4c(c3)C(c3ccccc3)(c3ccccc3)c3ccccc3-4)cc2)cc1]

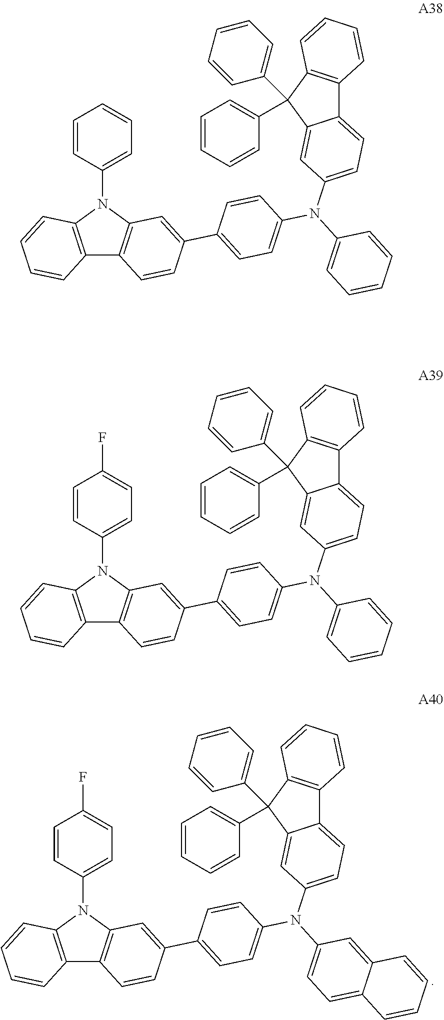 [SMILES: c1ccc(N(c2ccc(-c3ccc4c5ccccc5n(-c5ccccc5)c4c3)cc2)c2ccc3c(c2)C(c2ccccc2)(c2ccccc2)c2ccccc2-3)cc1 ; Fc1ccc(-n2c3ccccc3c3ccc(-c4ccc(N(c5ccccc5)c5ccc6c(c5)C(c5ccccc5)(c5ccccc5)c5ccccc5-6)cc4)cc32)cc1 ; Fc1ccc(-n2c3ccccc3c3ccc(-c4ccc(N(c5ccc6c(c5)C(c5ccccc5)(c5ccccc5)c5ccccc5-6)c5ccc6ccccc6c5)cc4)cc32)cc1]

In addition, the light emitting device ED of an embodiment may further include a material of a hole transport region further described below in the hole transport region HTR in addition to the amine compound of an embodiment and the amine derivative compound represented by Formula 10, described above.

The hole transport region HTR may include a compound represented by Formula H-1 below.

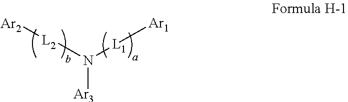

In Formula H-1 above, L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. “a” and “b” may be each independently an integer of 0 to 10. In some embodiments, if “a” or “b” is an integer of 2 or more, multiple L₁ and L₂ may be each independently a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. In addition, in Formula H-1, Ar₃ may be a substituted or unsubstituted aryl group of 6 to 30 ring-forming carbon atoms.

The compound represented by Formula H-1 may be a monoamine compound. Otherwise, the compound represented by Formula H-1 may be a diamine compound in which at least one selected from among Ar₁ to Ar₃ includes an amine group as a substituent. In addition, the compound represented by Formula H-1 may be a carbazole-based compound in which at least one selected from among Ar₁ and Ar₂ includes a substituted or unsubstituted carbazole group, or a fluorene-based compound in which at least one selected from among Ar₁ and Ar₂ includes a substituted or unsubstituted fluorene group.

The compound represented by Formula H-1 may be represented by any one selected from among the compounds in Compound Group H below. However, the compounds shown in Compound Group H are only examples, and the compound represented by Formula H-1 is not limited to the compounds represented in Compound Group H below.

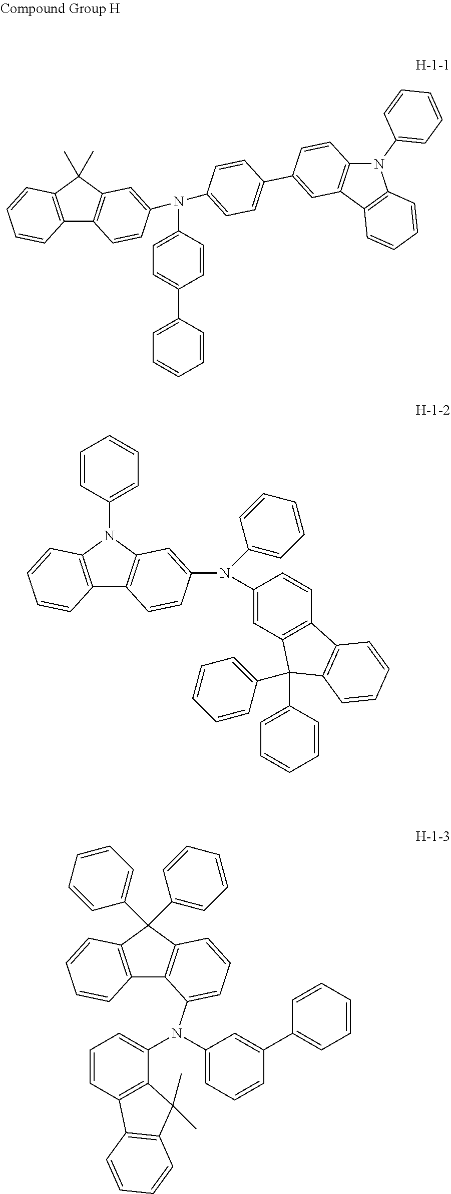 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

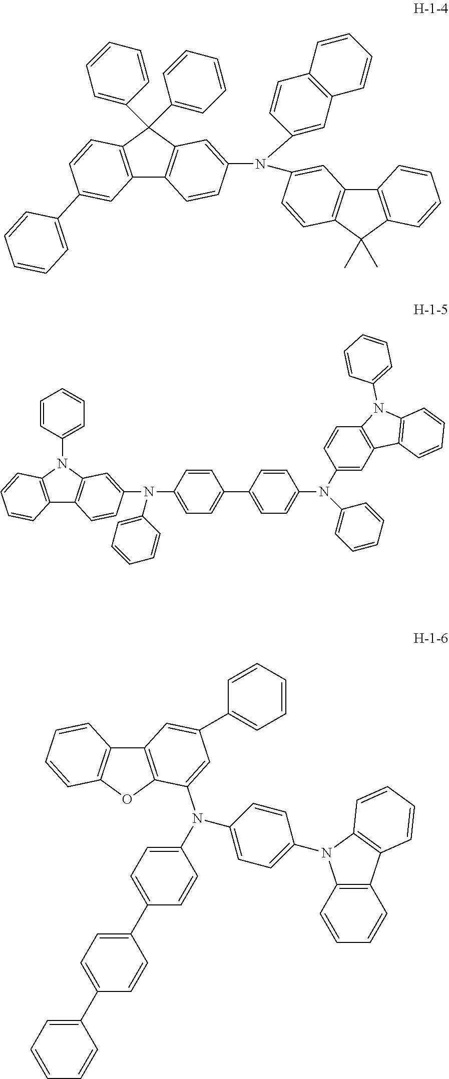 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

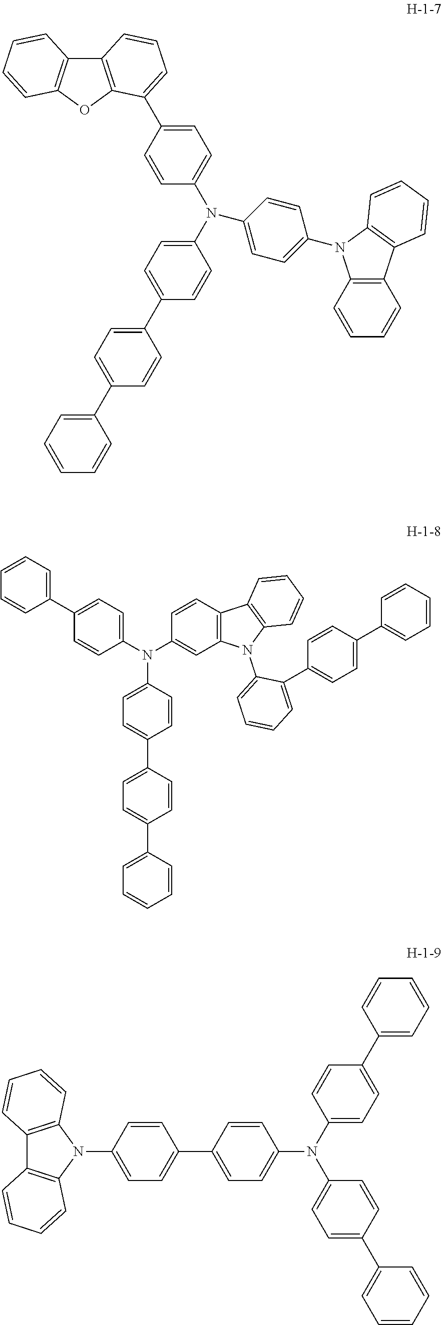 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

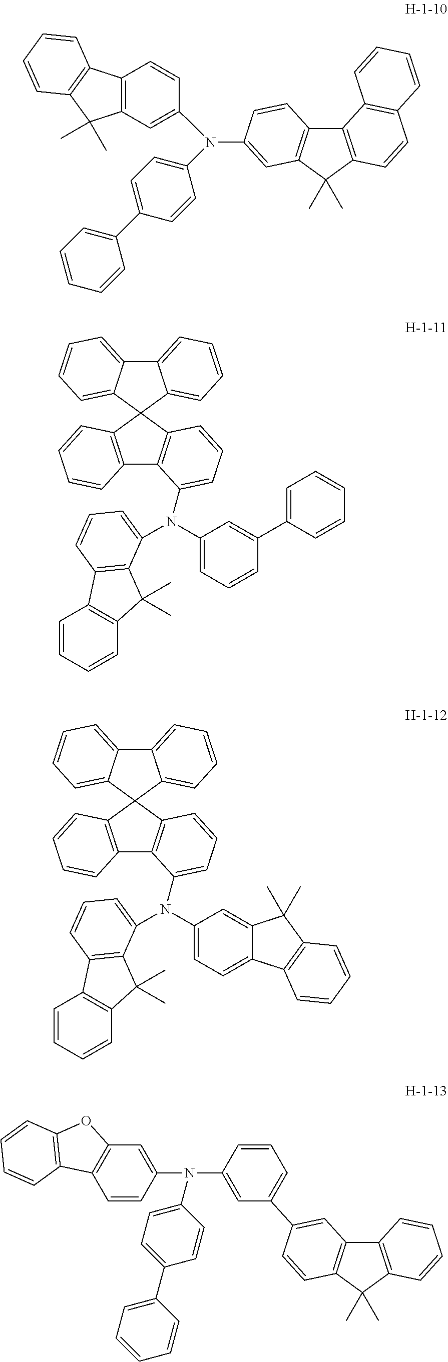

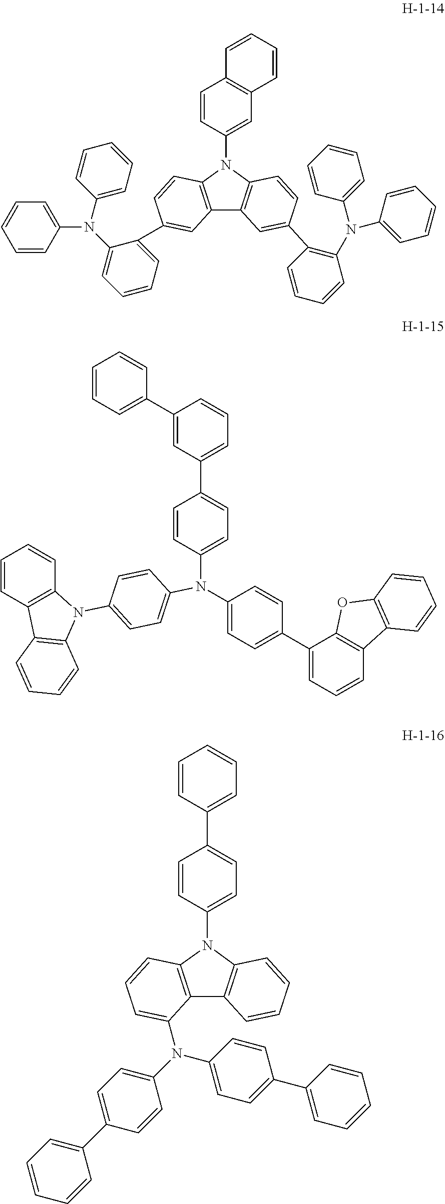 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

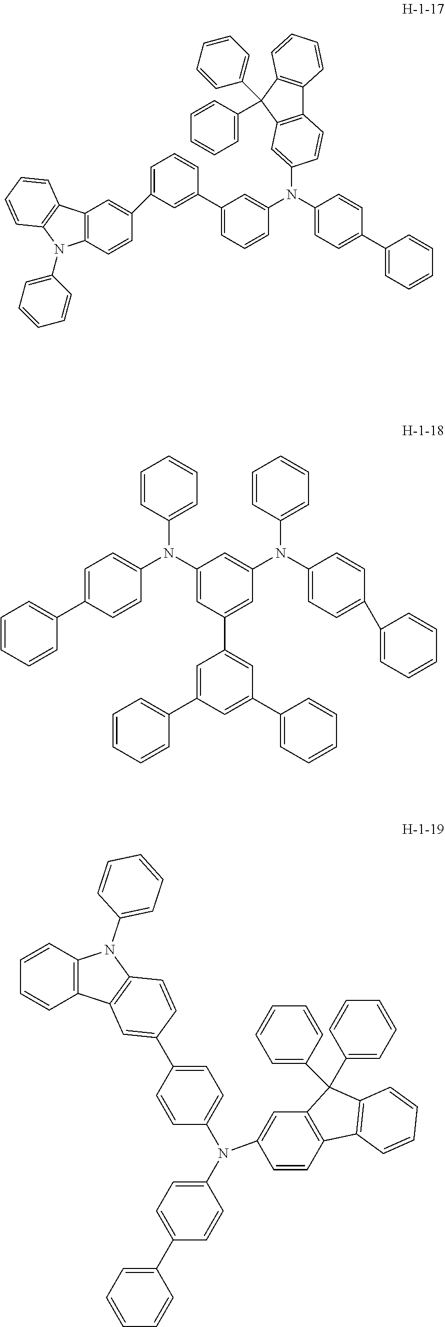 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In addition, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one selected from among the hole injection layer HIL, hole transport layer HTL, and electron blocking layer EBL.

The thickness of the hole transport region HTR may be from about 100 Å to about 10,000 Å, for example, from about 100 Å to about 5,000 Å. If the hole transport region HTR includes a hole injection layer HIL, the thickness of the hole injection region HIL may be, for example, from about 30 Å to about 1,000 Å. If the hole transport region HTR includes a hole transport layer HTL, the thickness of the hole transport layer HTL may be from about 30 Å to about 1,000 Å. For example, if the hole transport region HTR includes an electron blocking layer, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy the above-described ranges, suitable or satisfactory hole transport properties may be achieved without substantial increase of a driving voltage.

The hole transport region HTR may be formed by various suitable methods such as a vacuum deposition method, a spin coating method, a cast method, a Langmuir-Blodgett (LB) method, an ink jet printing method, a laser printing method, and/or a laser induced thermal imaging (LITI) method.

The hole transport region HTR may further include a charge generating material to increase conductivity (e.g., electrical conductivity) in addition to the above-described materials. The charge generating material may be dispersed uniformly or non-uniformly in the hole transport region HTR. The charge generating material may be, for example, a p-dopant. The p-dopant may include at least one selected from metal halide compounds, quinone derivatives, metal oxides, and cyano group-containing compounds, without limitation. For example, the p-dopant may include metal halide compounds such as CuI and/or RbI, quinone derivatives such as tetracyanoquinodimethane (TCNQ) and/or 2,3,5,6-tetrafluoro-7,7′,8,8-tetracyanoquinodimethane (F4-TCNQ), metal oxides such as tungsten oxide and/or molybdenum oxide, cyano group-containing compounds such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include at least one selected from among a buffer layer and an electron blocking layer EBL in addition to the hole injection layer HIL and the hole transport layer HTL. The buffer layer may compensate resonance distance according to the wavelength of light emitted from an emission layer EML and may increase emission efficiency. As materials included in the buffer layer, materials which may be included in the hole transport region HTR may be used. The electron blocking layer EBL is a layer playing the role of blocking or reducing the injection of electrons from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using multiple different materials, or a multilayer structure having multiple layers formed using multiple different materials.

In the light emitting device ED of an embodiment, the emission layer EML may include anthracene derivatives, pyrene derivatives, fluoranthene derivatives, chrysene derivatives, dihydrobenzanthracene derivatives, and/or triphenylene derivatives. For example, the emission layer EML may include anthracene derivatives and/or pyrene derivatives.

In the light emitting devices ED of embodiments, shown in FIG. 3 to FIG. 8, the emission layer EML may include a host and a dopant, and the emission layer EML may include a compound represented by Formula E-1 below. The compound represented by Formula E-1 below may be used as a fluorescence host material.

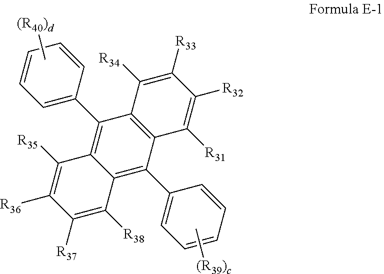

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In some embodiments, R₃₁ to R₄₀ may be combined with adjacent groups to form saturated hydrocarbon rings, unsaturated hydrocarbon rings, saturated heterocycles, or unsaturated heterocycles.

In Formula E-1, “c” and “d” may be each independently an integer of 0 to 5.

Formula E-1 may be represented by any one selected from among Compound E1 to Compound E19 below.

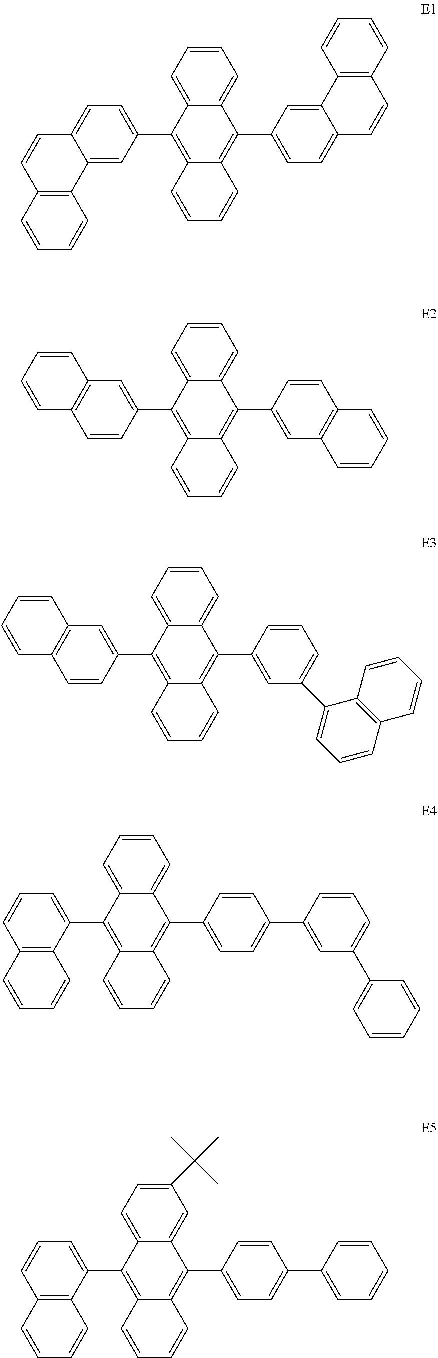

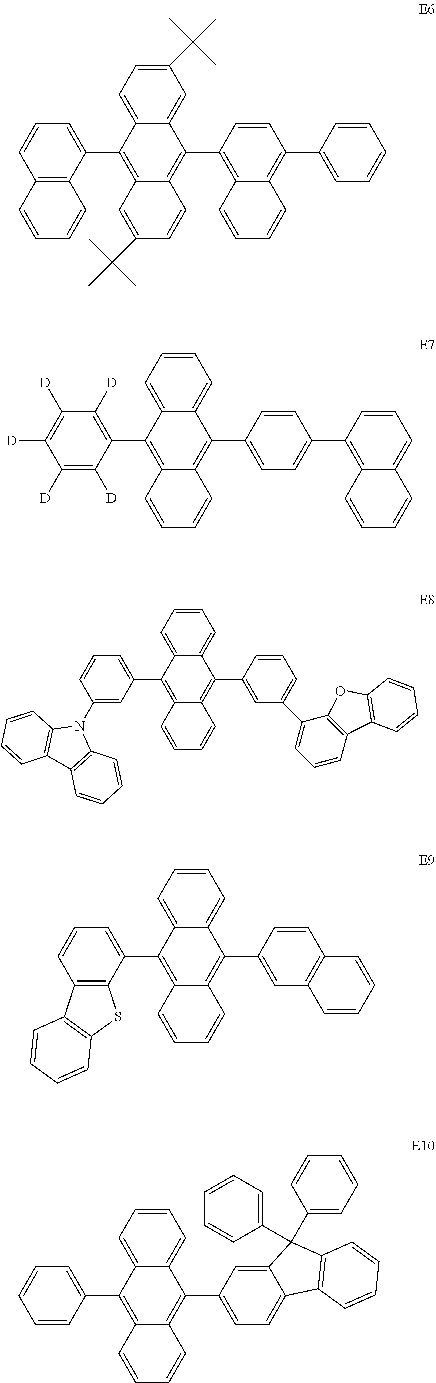

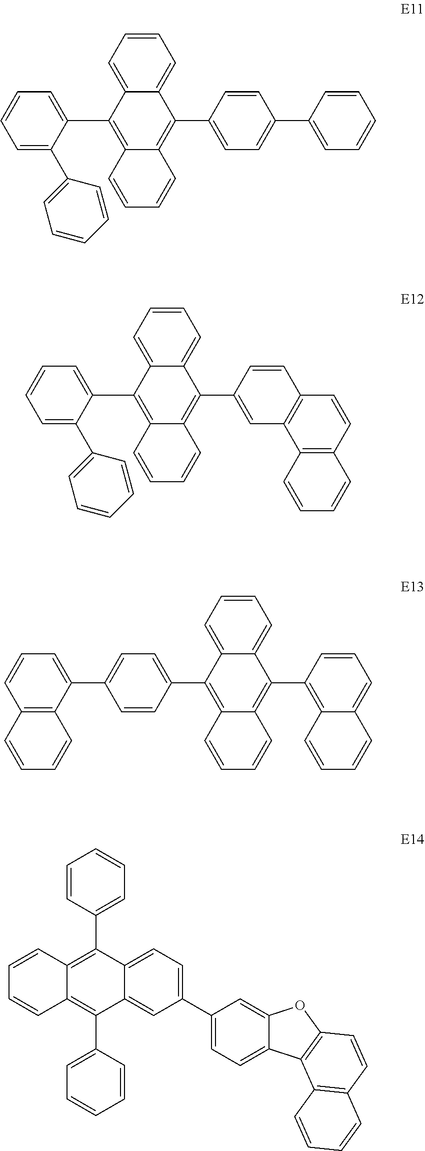

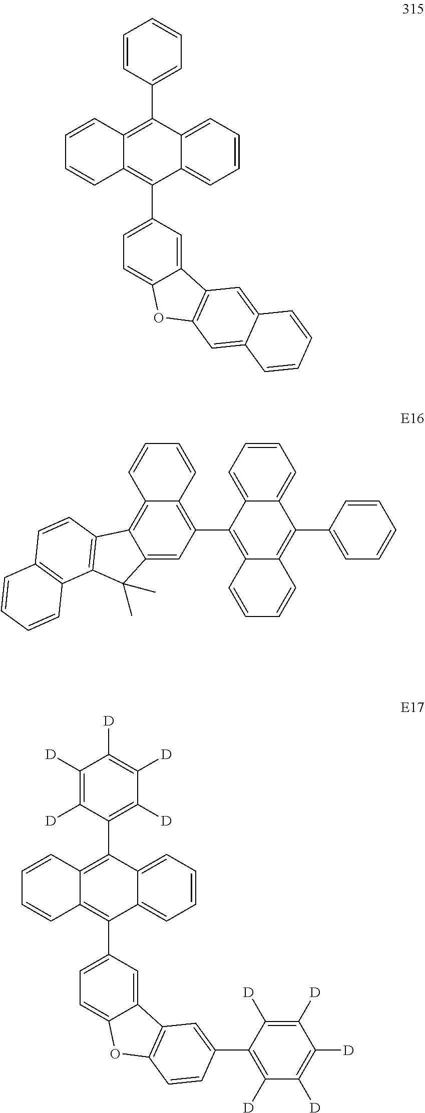

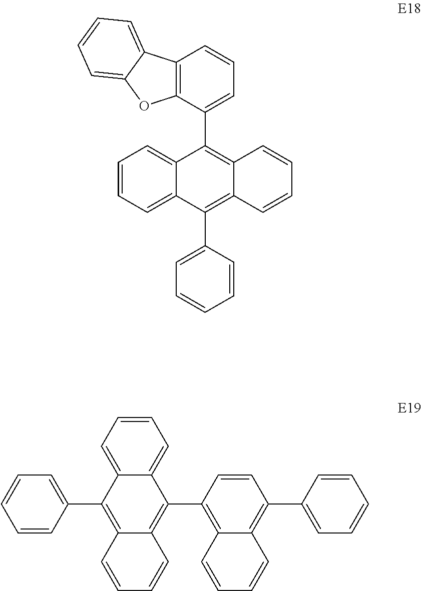

In an embodiment, the emission layer EML may include a compound represented by Formula E-2a or Formula E-2b below. The compound represented by Formula E-2a or Formula E-2b below may be used as a phosphorescence host material.

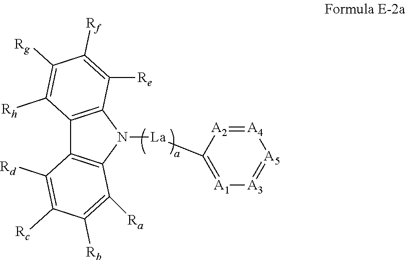

In Formula E-2a, “a” may be an integer of 0 to 10, Lₐ may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, if “a” is an integer of 2 or more, multiple Lₐ may be each independently a substituted or unsubstituted arylene group of 6 to 30 ring-forming carbon atoms, or a substituted or unsubstituted heteroarylene group of 2 to 30 ring-forming carbon atoms.

In addition, in Formula E-2a, A₁ to A₅ may be each independently N or CRi. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder may be CRᵢ.

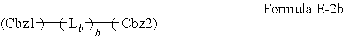

In Formula E-2b, Cbz1 and Cbz2 may be each independently an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b} may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2 below. However, the compounds shown in Compound Group E-2 below are only examples, and the compound represented by Formula E-2a or Formula E-2b is not limited to the compounds represented in Compound Group E-2 below.

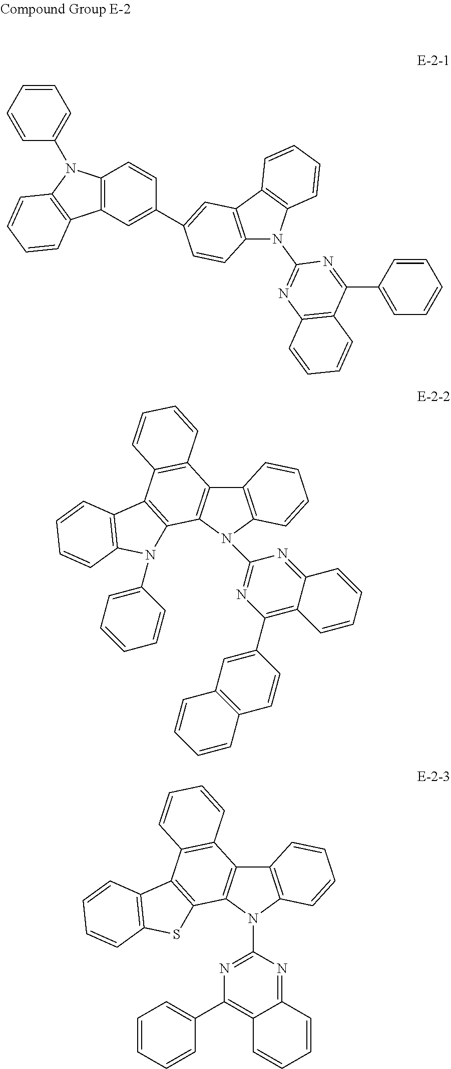

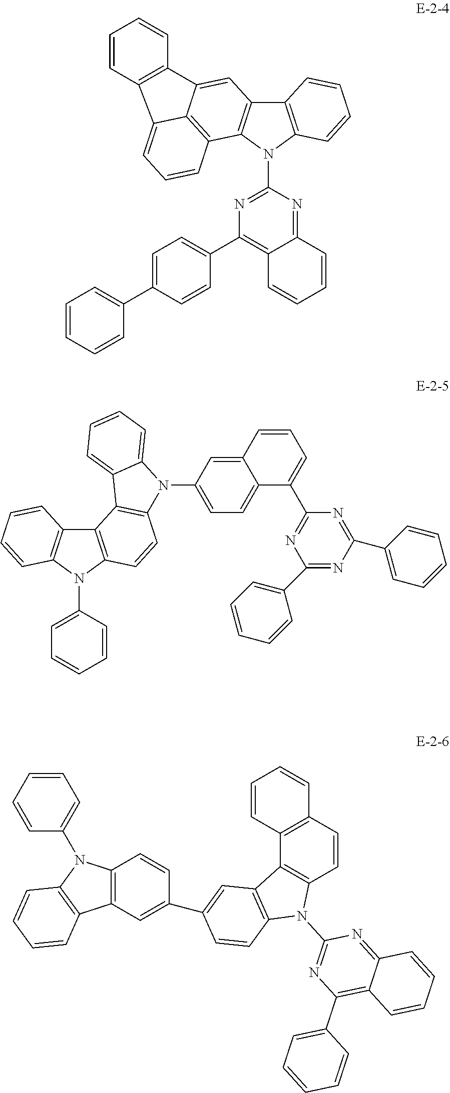 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

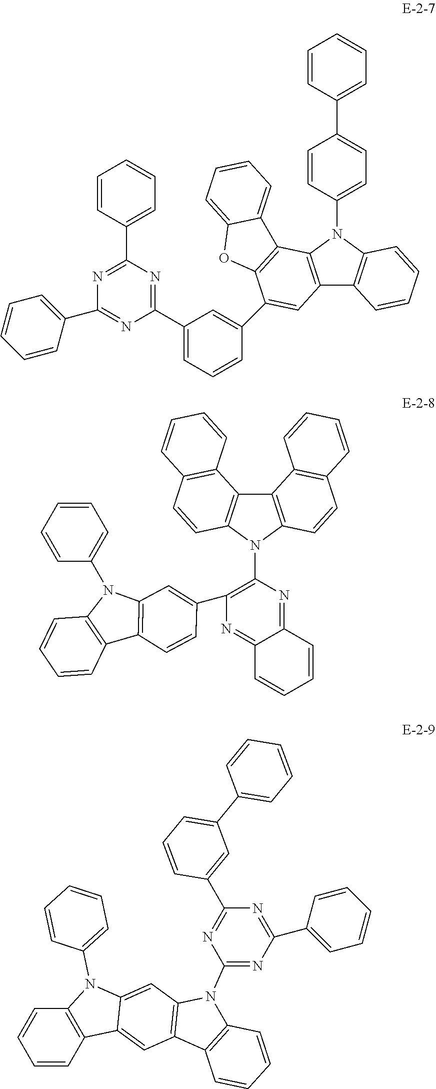

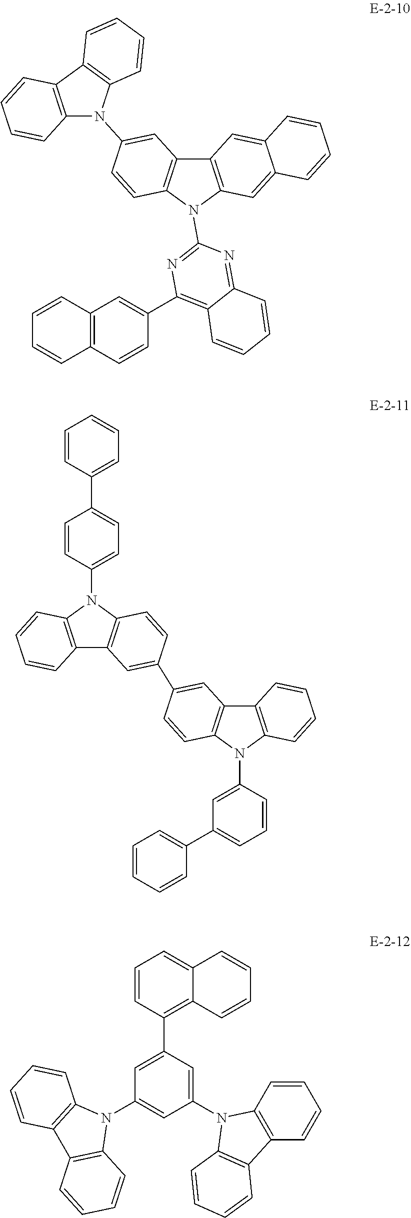 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

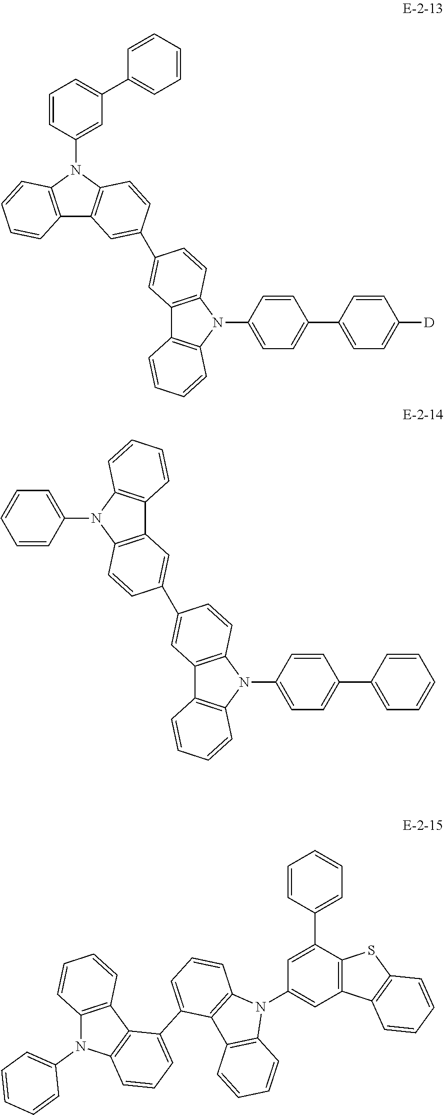 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

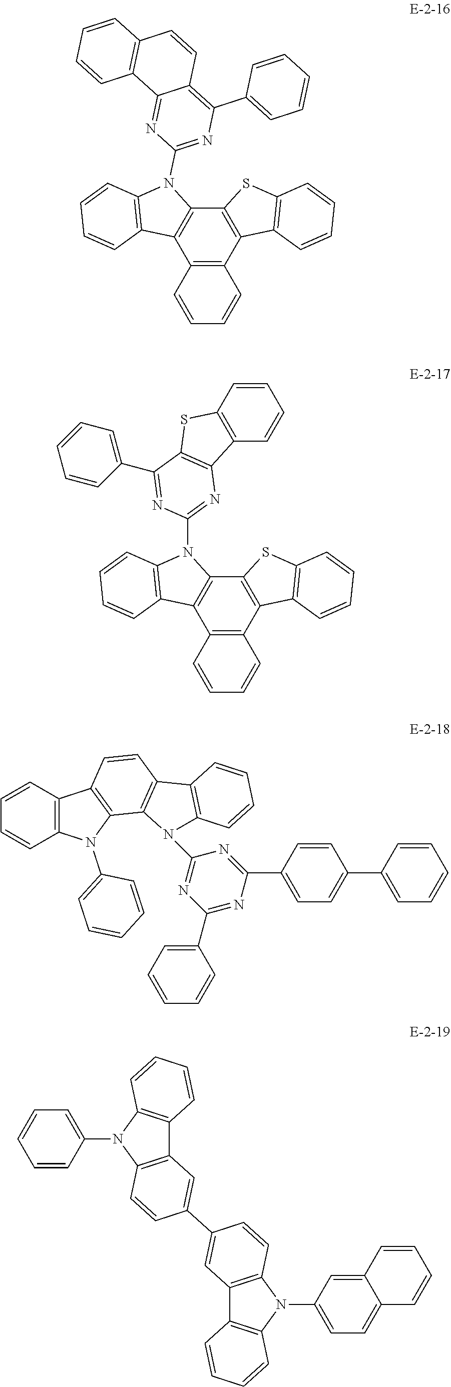 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

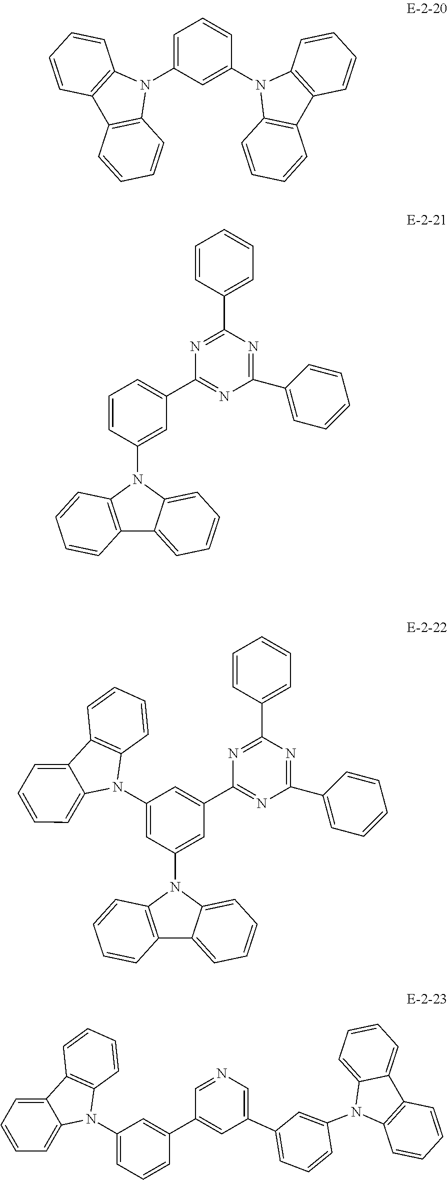 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

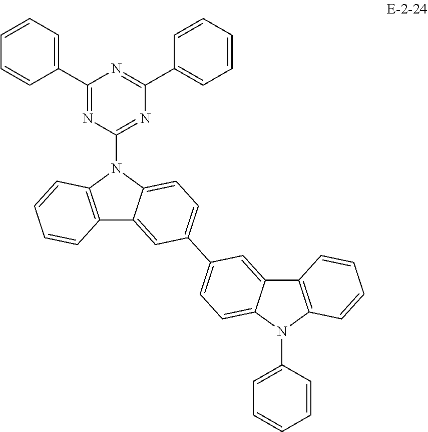 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include any suitable material generally used in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, an embodiment of the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b may be used as a phosphorescence dopant material.

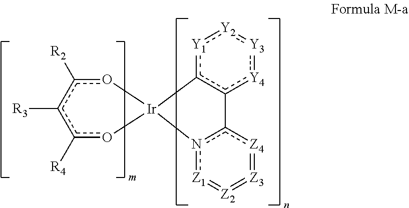

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be used as a phosphorescence dopant.

The compound represented by Formula M-a may be represented by any one selected from among Compounds M-a1 to M-a25 below. However, Compounds M-a1 to M-a25 below are examples, and the compound represented by Formula M-a is not limited to the compounds represented by Compounds M-a1 to M-a25 below.

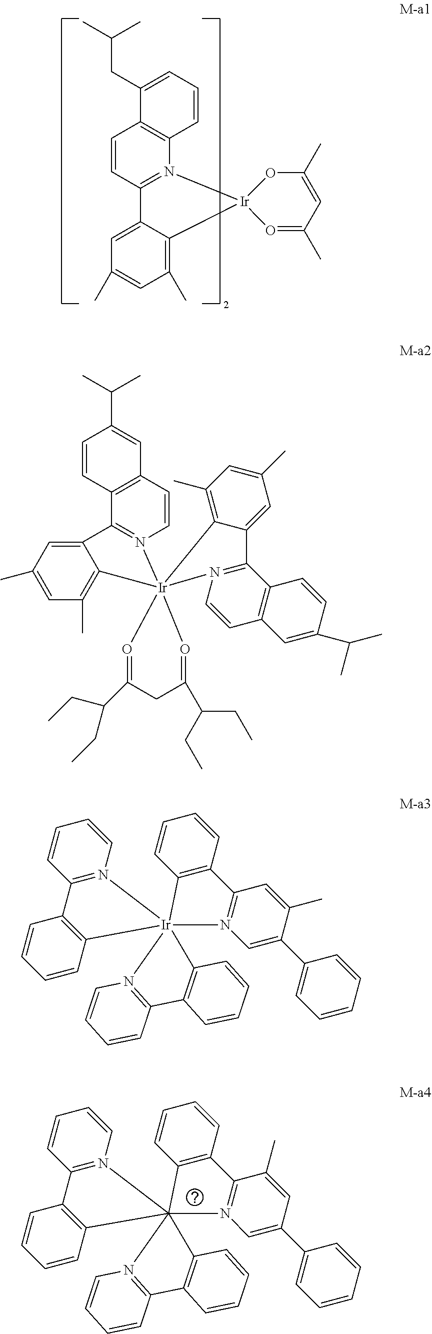

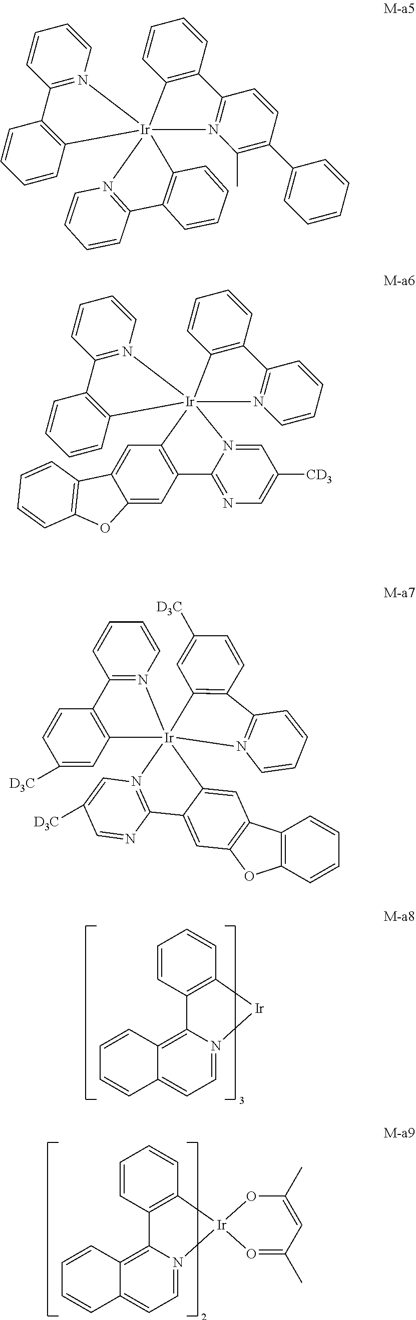

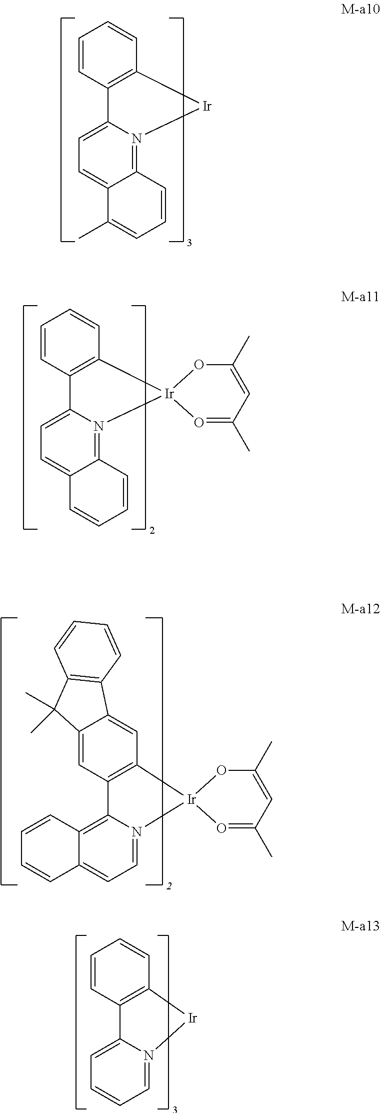

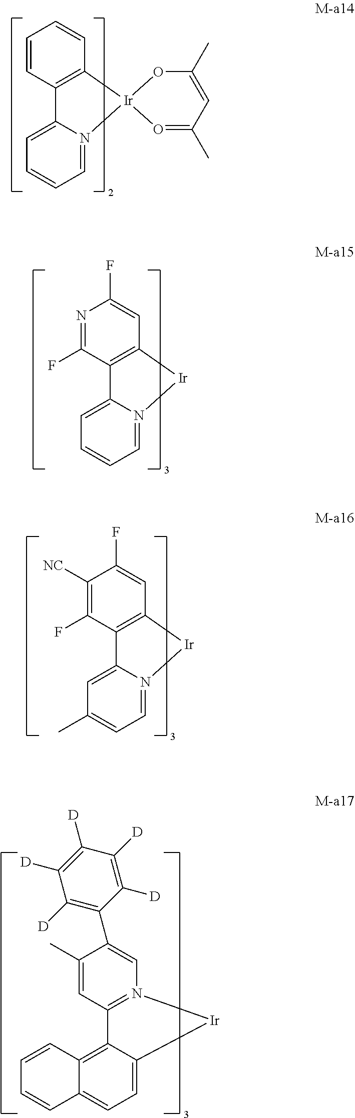

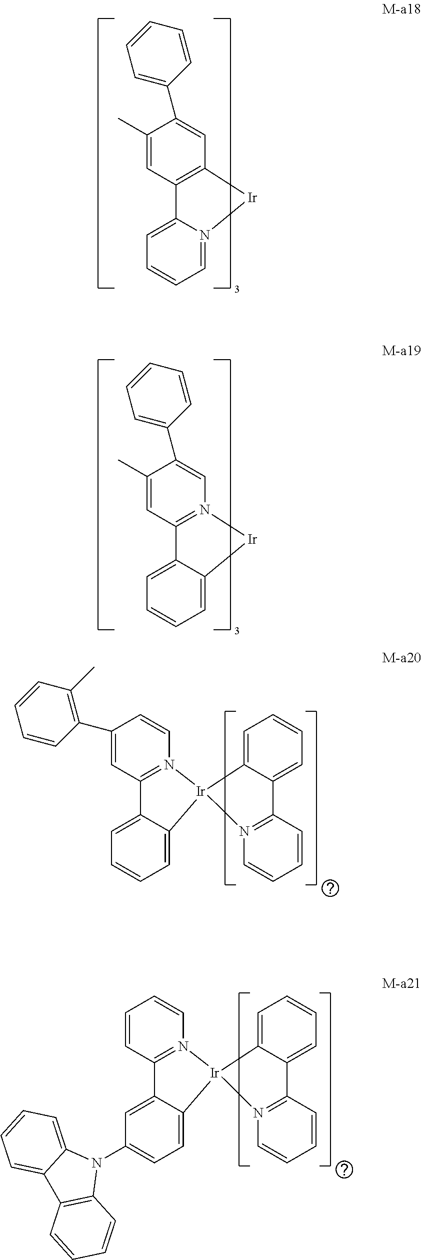

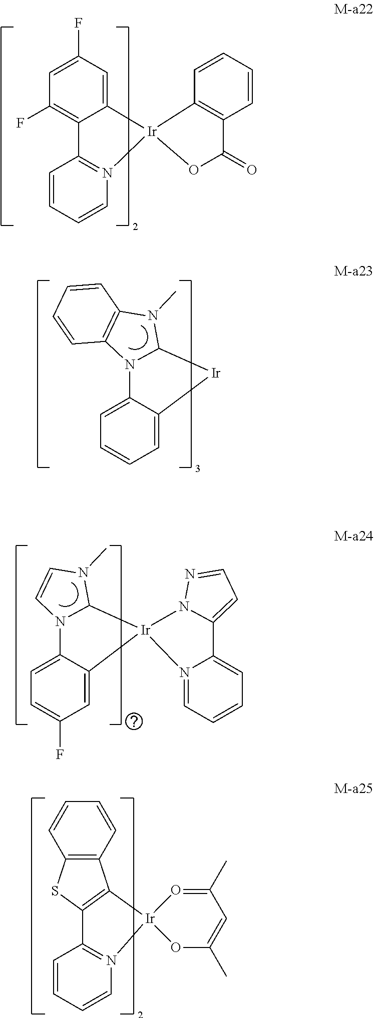

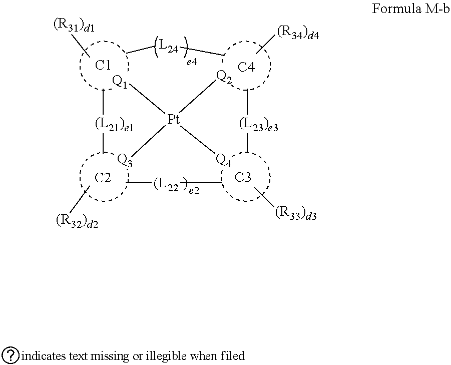

In Formula M-b, Q₁ to Q₄ are each independently C or N, C1 to C4 are each independently a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ are each independently a direct linkage,

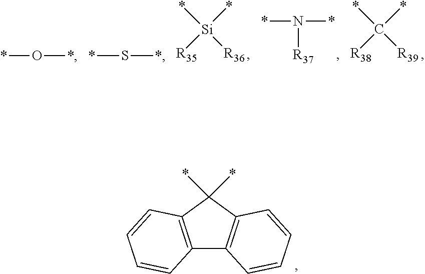

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 are each independently an integer of 0 to 4.

The compound represented by Formula M-b may be used as a blue phosphorescence dopant or a green phosphorescence dopant.

The compound represented by Formula M-b may be represented by any one selected from among the compounds below. However, the compounds below are examples, and the compound represented by Formula M-b is not limited to the compounds represented below.

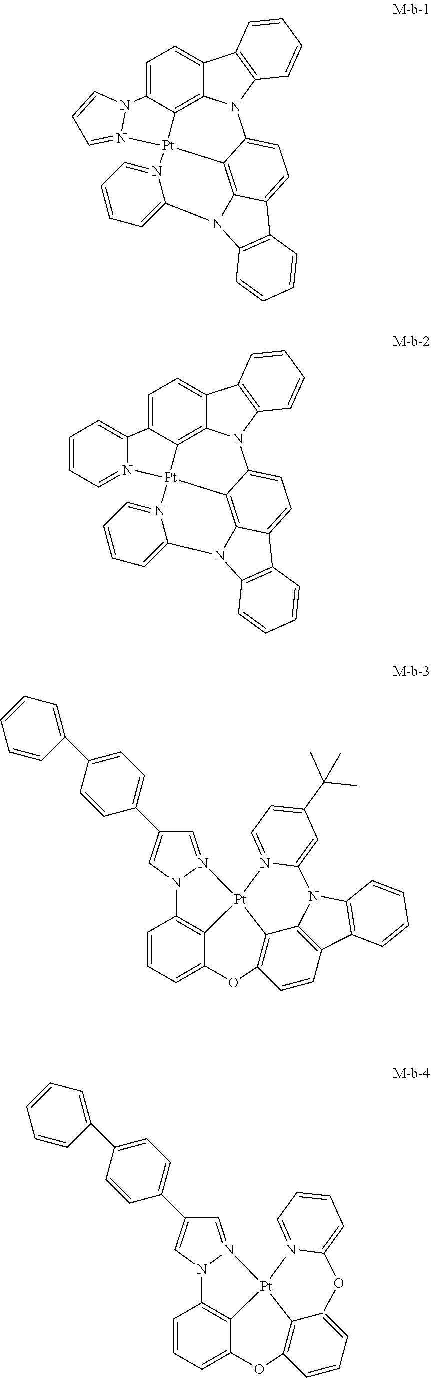

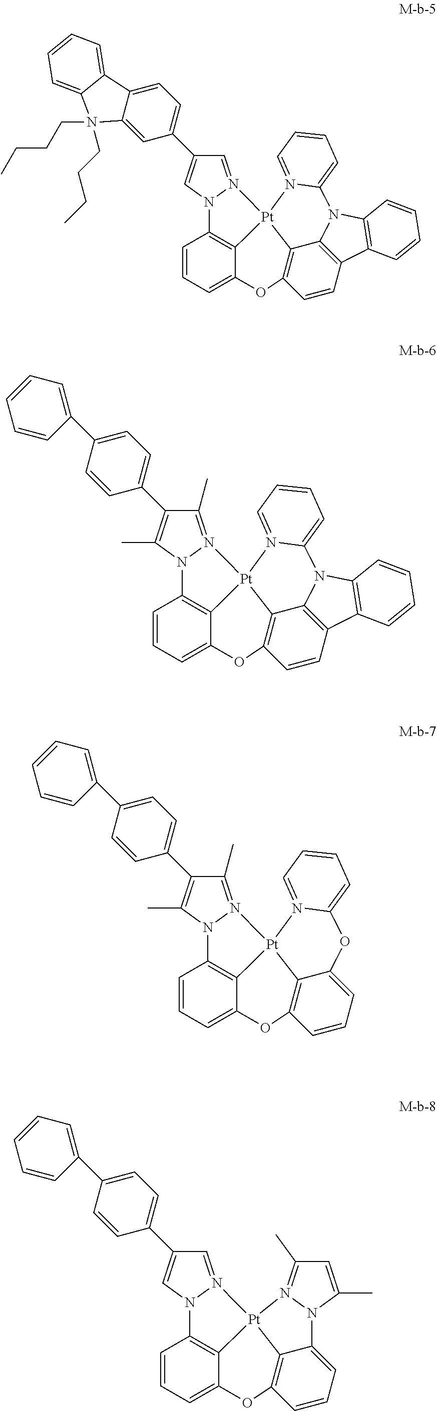

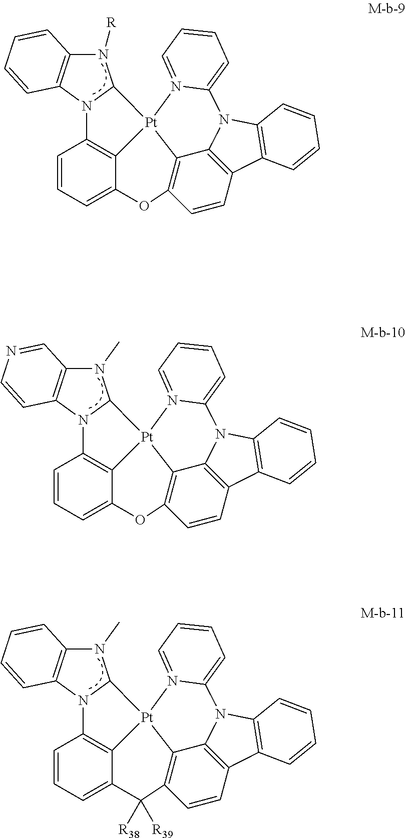

In the compounds above, R, R₃₈, and R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c below. The compounds represented by Formula F-a to Formula F-c below may be used as fluorescence dopant materials.

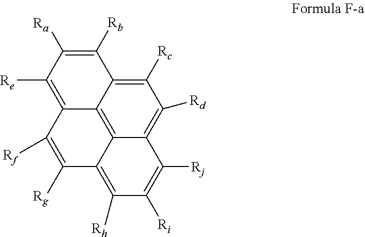

In Formula F-a, two selected from Rₐ to Rⱼ may be each independently substituted with

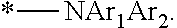

The remainder not substituted with

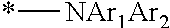

selected from among Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In

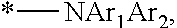

Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including O or S as a ring-forming atom.

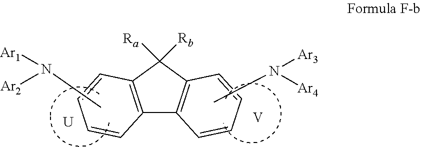

In Formula F-b, Rₐ and R_{b} may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms.

In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may be each independently 0 or 1. For example, in Formula F-b, if the number of U or V is 1, one ring forms a fused ring at the designated part by U or V, and if the number of U or V is 0, a ring is not present at the designated part by U or V. In some embodiments, if the number of U is 0, and the number of V is 1, or if the number of U is 1, and the number of V is 0, a fused ring having the fluorene core of Formula F-b may be a ring compound having four rings. In addition, if the number of both U and V is 0, the fused ring of Formula F-b may be a ring compound having three rings. In addition, if the number of both U and V is 1, a fused ring having the fluorene core of Formula F-b may be a ring compound having five rings.

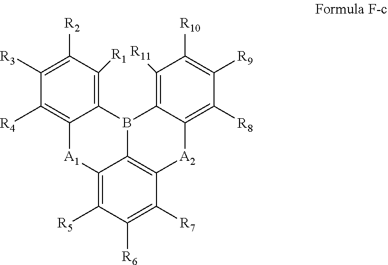

In Formula F-c, A₁ and A₂ may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may be each independently combined with the substituents of an adjacent ring to form a fused ring. For example, if A₁ and A₂ may be each independently NRₘ, A₁ may be combined with R₄ or R₅ to form a ring. In addition, A₂ may be combined with R₇ or R₈ to form a ring.

In an embodiment, the emission layer EML may include any suitable dopant material generally used in the art. In some embodiments, the emission layer EML may include styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may include any suitable phosphorescence dopant material generally used in the art. In some embodiments, the phosphorescence dopant may include a metal complex including iridium (Ir), platinum (Pt), osmium (Os), gold (Au), titanium (Ti), zirconium (Zr), hafnium (Hf), europium (Eu), terbium (Tb) or thulium (Tm). In some embodiments, iridium(III) bis(4,6-difluorophenylpyridinato-N,C2′)picolinate (Flrpic), bis(2,4-difluorophenylpyridinato)-tetrakis(1-pyrazolyl)borate (Flr6), or platinum octaethyl porphyrin (PtOEP) may be used as the phosphorescence dopant. However, an embodiment of the present disclosure is not limited thereto.

The emission layer EML may include a quantum dot material. The core of the quantum dot may be selected from Group II-VI compounds, Group III-VI compounds, Group I-III-VI compounds, Group III-V compounds, Group III-II-V compounds, Group IV-VI compounds, Group IV elements, Group IV compounds, and combinations thereof.

The Group II-VI compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The Group III-VI compound may include a binary compound such as In₂S₃, and In₂Se₃, a ternary compound such as InGaS₃, and InGaSe₃, or optional combinations thereof.

The Group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the Group III-V compound may further include a II group metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The Group IV element may be selected from the group consisting of Si, Ge, and a mixture thereof. The Group IV group may be a binary compound selected from the group consisting of SiC, SiGe, and a mixture thereof.

In this case, the binary compound, the ternary compound or the quaternary compound may be present at a uniform concentration in a particle or may be present at a partially different concentration distribution state in the same particle. In addition, a core/shell structure in which one quantum dot wraps another quantum dot may be possible. The interface of the core and the shell may have a concentration gradient in which the concentration of an element present in the shell is decreased along a direction toward the center.

In some embodiments, the quantum dot may have the above-described core-shell structure including a core including a nanocrystal and a shell wrapping the core. The shell of the quantum dot may play the role of a protection layer for preventing or reducing the chemical deformation or degradation of the core to maintain semiconductor properties and/or a charging layer for imparting the quantum dot having electrophoretic properties. The shell may have a single layer or a multilayer. Examples of the shell of the quantum dot may include a metal or non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but an embodiment of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but an embodiment of the present disclosure is not limited thereto.

The quantum dot may have a full width of half maximum (FWHM) of emission wavelength spectrum of about 45 nm or less, about 40 nm or less, or, for example, about 30 nm or less. Within this range, color purity or color reproducibility may be improved. In addition, light emitted via such quantum dot is emitted in all (e.g., substantially all) directions, and light view angle properties may be improved.

In addition, the shape of the quantum dot may be any generally used shapes in the art, without specific limitation. In some embodiments, the shape of spherical, pyramidal, multi-arm, or cubic nanoparticle, nanotube, nanowire, nanofiber, nanoplate particle, etc. may be used.

The quantum dot may control the color of light emitted according to the particle size of the quantum dot, and accordingly, the quantum dot may have various suitable emission colors such as blue, red and green.

In the light emitting devices ED of embodiments, as shown in FIG. 3 to FIG. 6, the electron transport region ETR is provided on the emission layer EML. The electron transport region ETR may include at least one of an electron blocking layer HBL, an electron transport layer ETL or an electron injection layer EIL. However, an embodiment of the present disclosure is not limited thereto.

The electron transport region ETR may have a single layer formed using a single material, a single layer formed using multiple different materials, or a multilayer structure having multiple layers formed using multiple different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below.

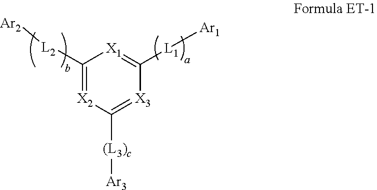

In Formula ET-1, at least one selected from among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may be each independently an integer of 0 to 10. In Formula ET-1, L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. In some embodiments, if “a” to “c” are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, an embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

The electron transport region ETR may include at least one selected from among Compounds ET1 to ET36 below.

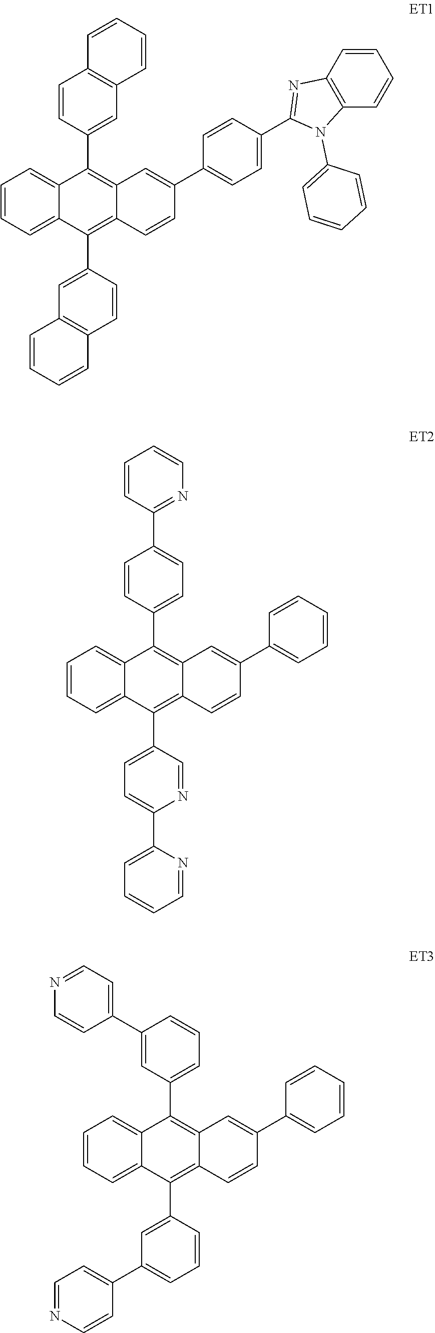 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)cc4)c3c2)cc1 ; c1ccc(-c2ccc3c(-c4cccc(-c5ccncc5)c4)c4ccccc4c(-c4cccc(-c5ccncc5)c4)c3c2)cc1]

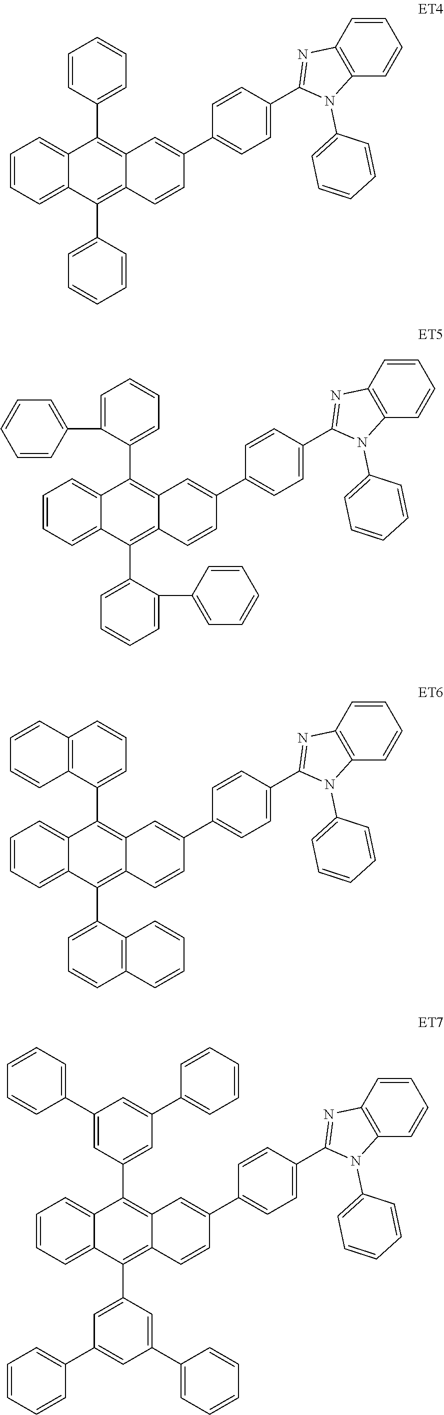 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

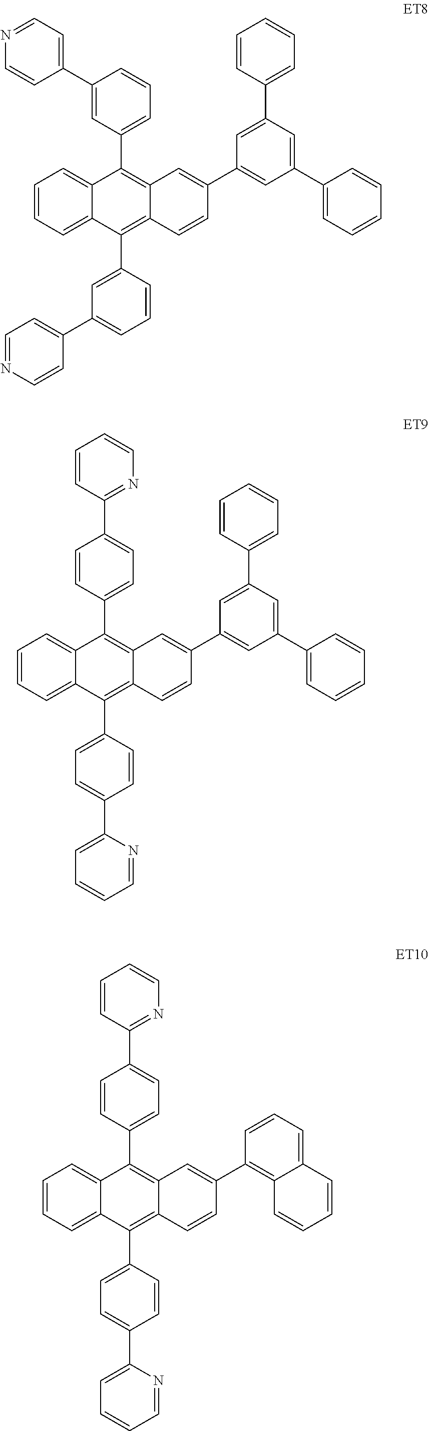 [SMILES: c1ccc(-c2cc(-c3ccccc3)cc(-c3ccc4c(-c5cccc(-c6ccncc6)c5)c5ccccc5c(-c5cccc(-c6ccncc6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

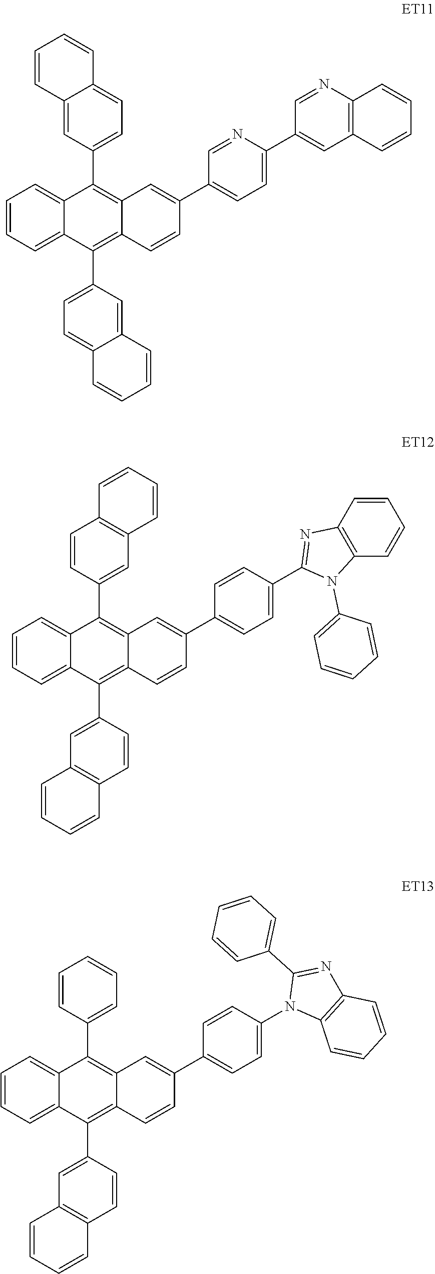 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

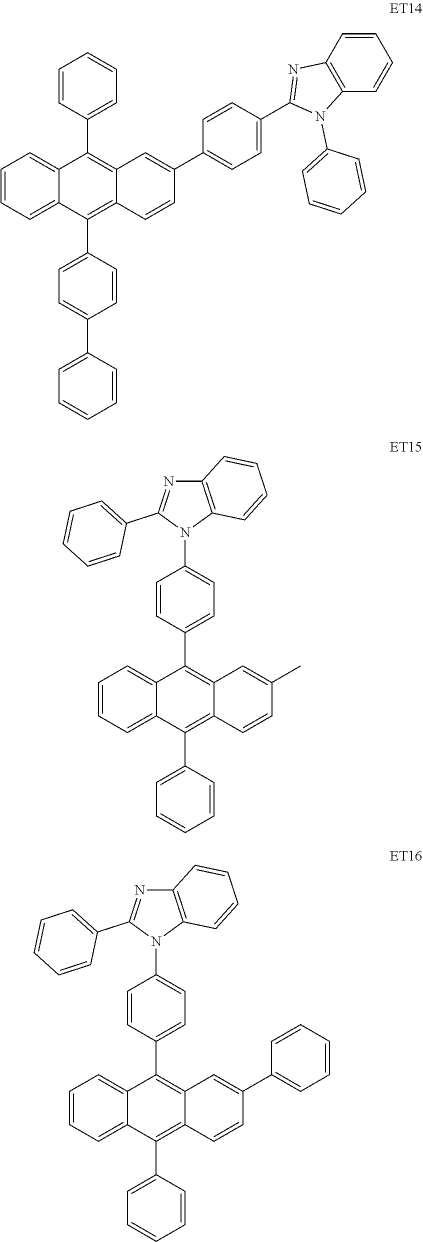

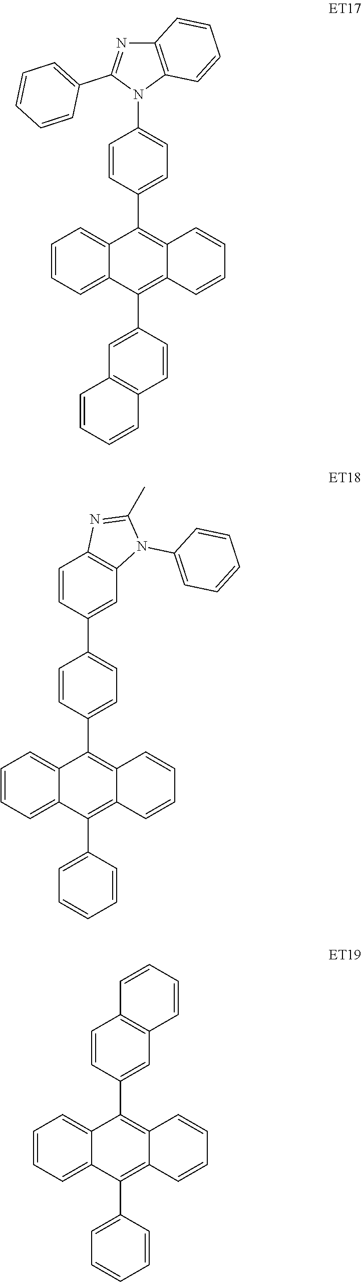

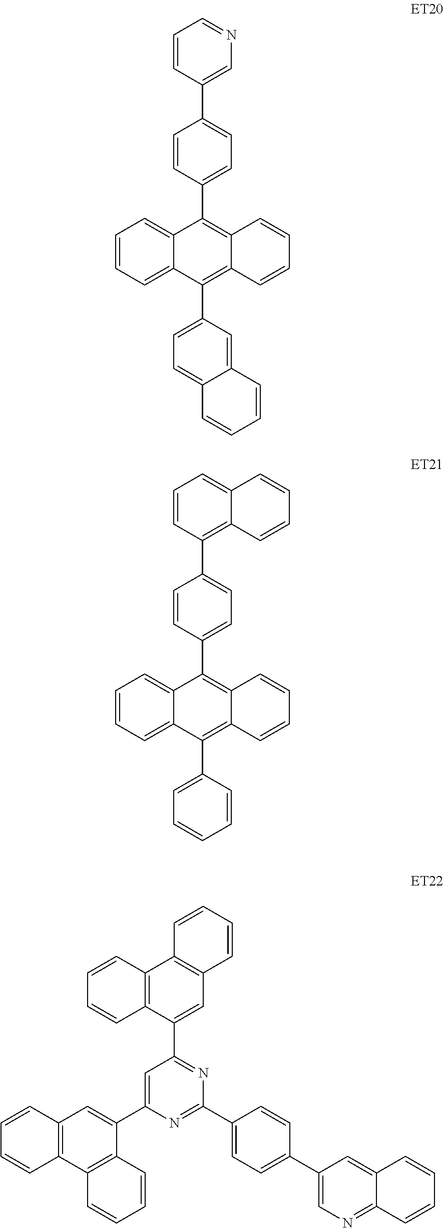

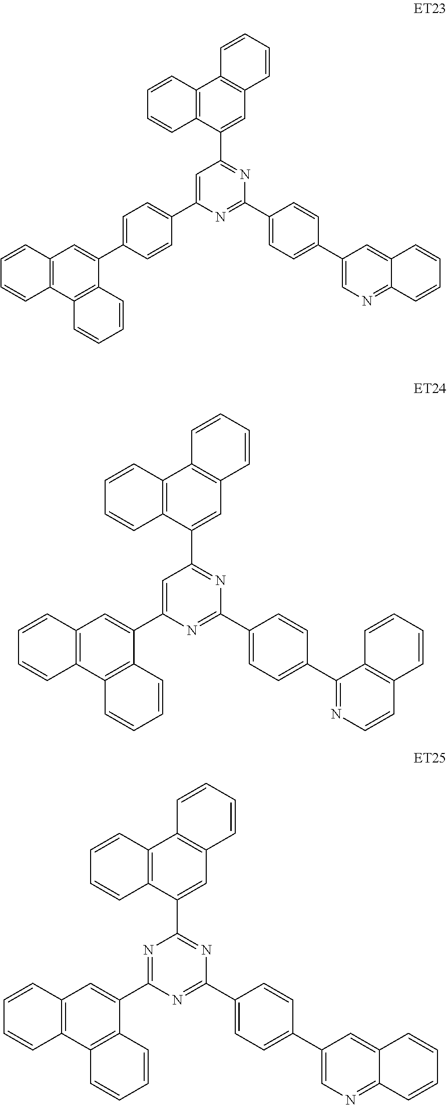 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

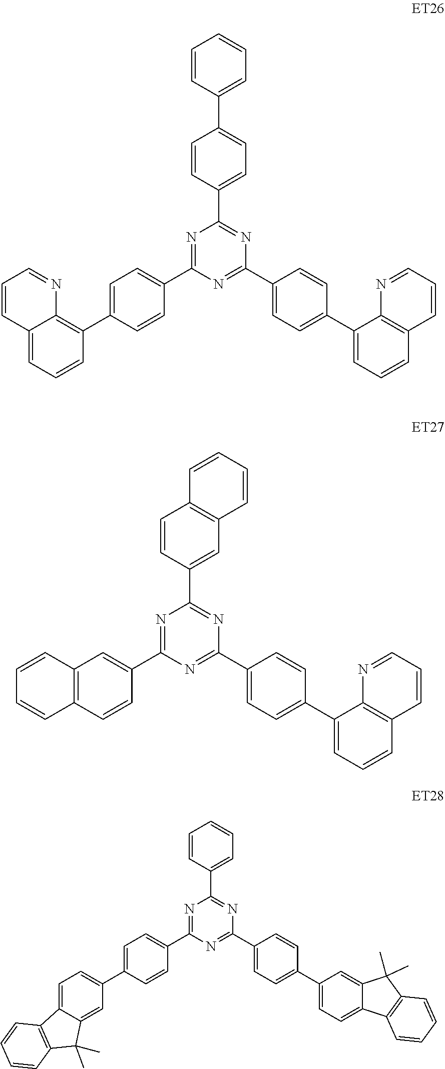 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

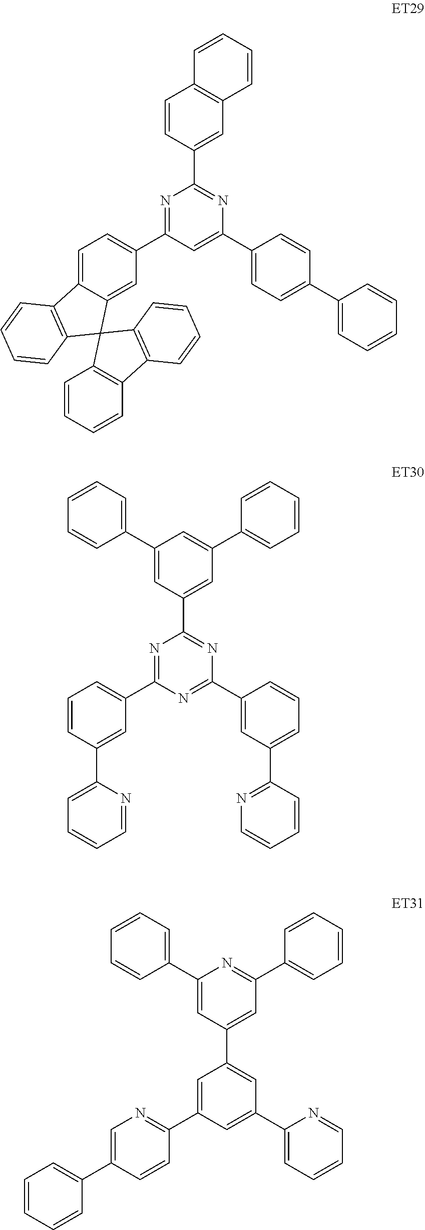 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

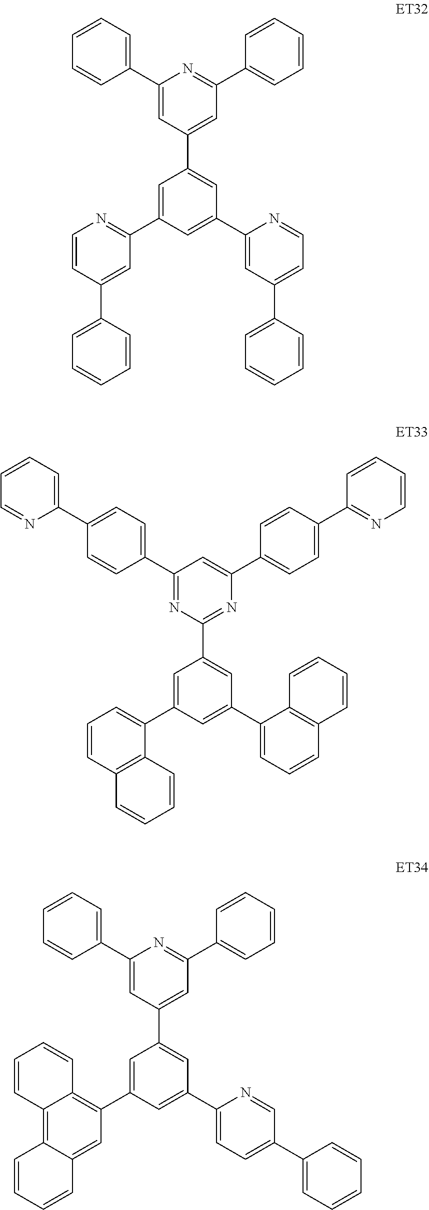 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

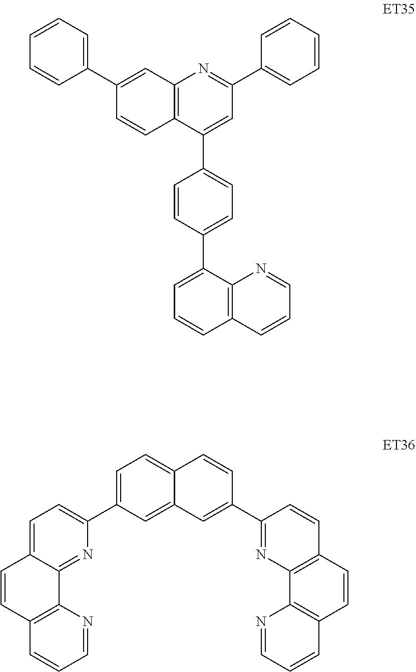 [SMILES: c1ccc(-c2ccc3c(-c4ccc(-c5cccc6cccnc56)cc4)cc(-c4ccccc4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI and KI, a metal in lanthanides such as Yb, or a co-deposited material of the metal halide and the metal in lanthanides. For example, the electron transport region ETR may include KI:Yb, RbI:Yb, LiF:Yb, etc., as the co-deposited material. In some embodiments, the electron transport region ETR may use a metal oxide such as Li₂O and BaO, or 8-hydroxy-lithium quinolate (Liq). However, an embodiment of the present disclosure is not limited thereto. The electron transport region ETR also may be formed using a mixture material of an electron transport material and an insulating organo metal salt. The organo metal salt may be a material having an energy band gap of about 4 eV or more. In some embodiments, the organo metal salt may include, for example, metal acetates, metal benzoates, metal acetoacetates, metal acetylacetonates, and/or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the aforementioned materials. However, an embodiment of the present disclosure is not limited thereto.

The electron transport region ETR may include the compounds of the electron transport region in at least one selected from among an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies the above-described range, suitable or satisfactory electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the above described range, suitable or satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but an embodiment of the present disclosure is not limited thereto. For example, if the first electrode EL1 is an anode, the second cathode EL2 may be a cathode, and if the first electrode EL1 is a cathode, the second electrode EL2 may be an anode. The second electrode may include at least one selected from Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, or oxides thereof.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds including thereof, or mixtures thereof (for example, AgMg, AgYb, or MgAg). Otherwise, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

In some embodiments, the second electrode EL2 may be connected with an auxiliary electrode. If the second electrode EL2 is connected with the auxiliary electrode, the resistance of the second electrode EL2 may decrease.

A capping layer CPL may be further on the second electrode EL2 in the light emitting device ED of an embodiment. The capping layer CPL may include a multilayer or a single layer.

In an embodiment, the capping layer CPL may be an organic layer or an inorganic layer. For example, if the capping layer CPL includes an inorganic material, the inorganic material may include an alkali metal compound such as LiF, an alkaline earth metal compound such as MgF₂, SiON, SiNx, SiOy, etc.

For example, if the capping layer CPL includes an organic material, the organic material may include 2,2′-dimethyl-N,N′-di-[(1-naphthyl)-N,N′-diphenyl]-1,1′-biphenyl-4,4′-diamine (α-NPD), NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., an epoxy resin, and/or acrylate such as methacrylate. In addition, a capping layer CPL may include at least one selected from among Compounds P1 to P5 below, but an embodiment of the present disclosure is not limited thereto.

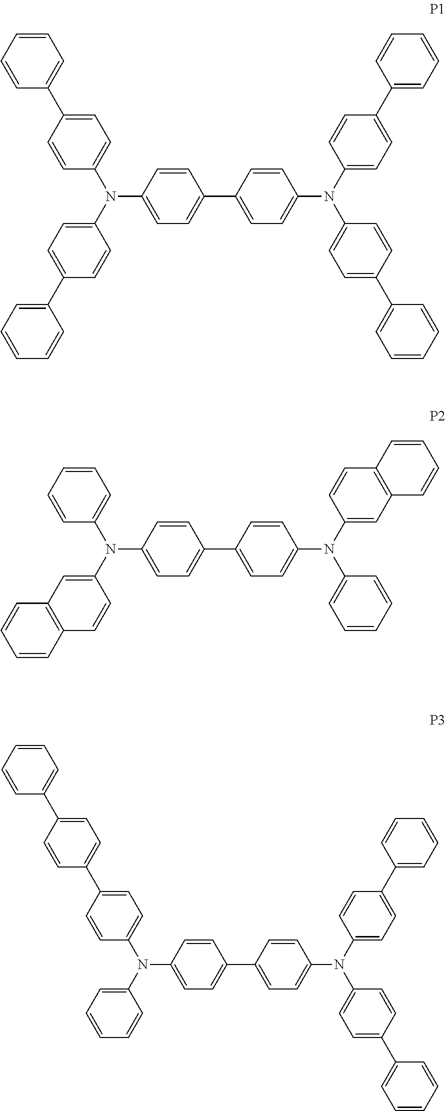 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

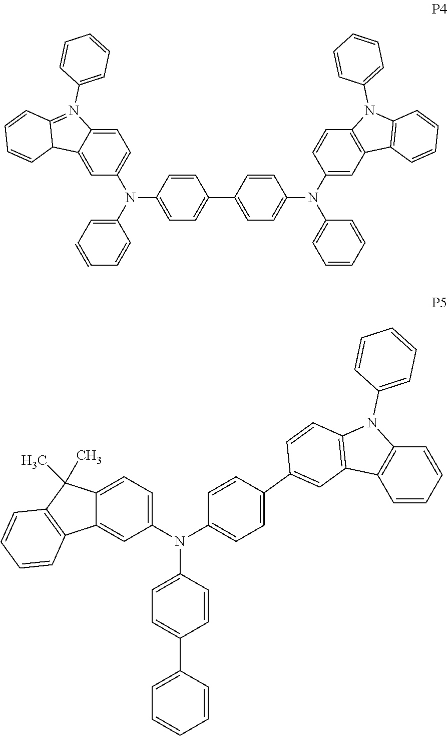

In some embodiments, the refractive index of the capping layer CPL may be about 1.6 or more. For example, the refractive index of the capping layer CPL with respect to light in a wavelength range of about 550 nm to about 660 nm may be about 1.6 or more.

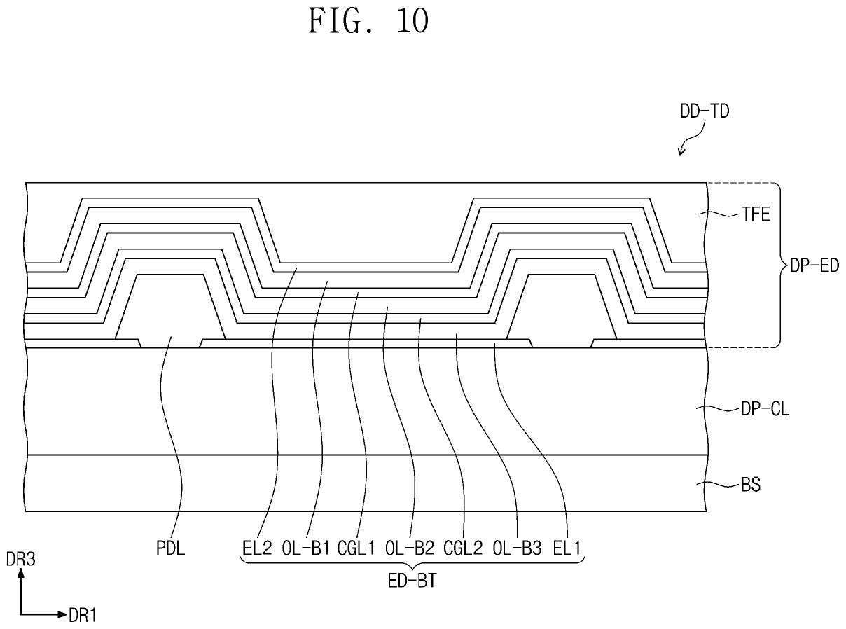

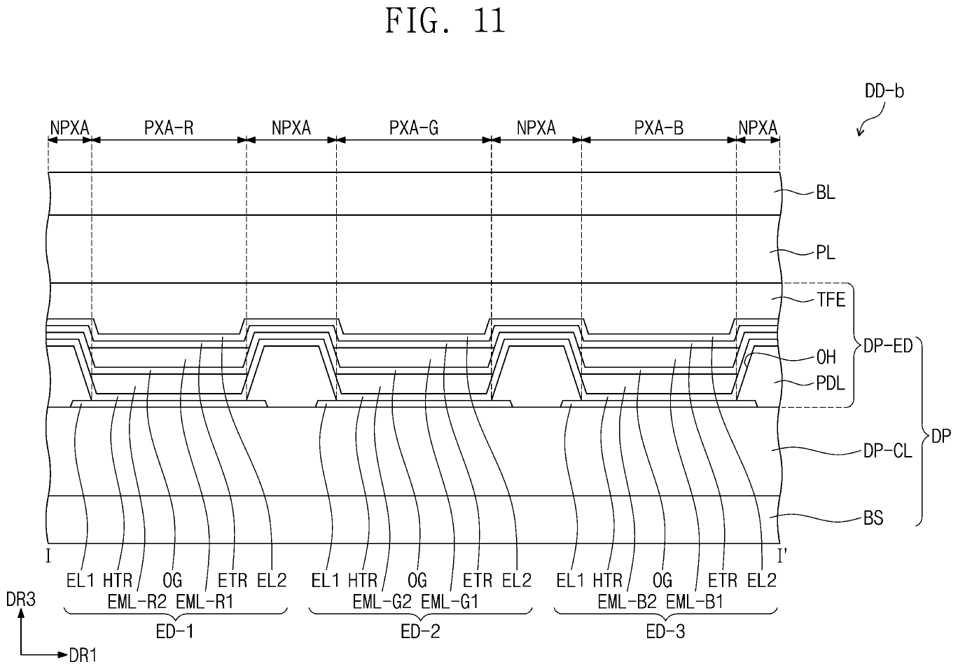

FIG. 9 and FIG. 10 are cross-sectional views on display apparatuses according to embodiments, respectively. In the explanation on the display apparatuses of embodiments, referring to FIG. 9 and FIG. 10, duplicative description of FIG. 1 to FIG. 8 will not be repeated here, and the different features will be explained chiefly.

Referring to FIG. 9, the display apparatus DD according to an embodiment may include a display panel DP including a display device layer DP-ED, a light controlling layer CCL on the display panel DP and a color filter layer CFL.

In an embodiment shown in FIG. 9, the display panel DP includes a base layer BS, a circuit layer DP-CL provided on the base layer BS and a display device layer DP-ED, and the display device layer DP-ED may include a light emitting device ED.

The light emitting device ED may include a first electrode EL1, a hole transport region HTR on the first electrode EL1, an emission layer EML on the hole transport region HTR, an electron transport region ETR on the emission layer EML, and a second electrode EL2 on the electron transport region ETR. The same structures of the light emitting devices of FIG. 3 to FIG. 8 may be applied to the structure of the light emitting device ED shown in FIG. 9.

Referring to FIG. 9, the emission layer EML may be in an opening part OH defined in a pixel definition layer PDL. For example, the emission layer EML divided by the pixel definition layer PDL and correspondingly provided to each of luminous areas PXA-R, PXA-G and PXA-B may emit light in the same (e.g., substantially the same) wavelength region. In the display apparatus DD of an embodiment, the emission layer EML may emit blue light. In some embodiments, the emission layer EML may be provided as a common layer for all luminous areas PXA-R, PXA-G and PXA-B.

The light controlling layer CCL may be on the display panel DP. The light controlling layer CCL may include a light converter. The light converter may be a quantum dot and/or a phosphor. The light converter may transform the wavelength of light provided and then emit light. In some embodiments, the light controlling layer CCL may be a layer including a quantum dot and/or a layer including a phosphor.

The light controlling layer CCL may include multiple light controlling parts CCP1, CCP2 and CCP3. The light controlling parts CCP1, CCP2 and CCP3 may be spaced apart from one another.

Referring to FIG. 9, a partition pattern BMP may be between the spaced apart light controlling parts CCP1, CCP2 and CCP3, but an embodiment of the present disclosure is not limited thereto. In FIG. 9, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 that converts a first color light provided from the light emitting device ED into a second color light, a second light controlling part CCP2 including a second quantum dot QD2 that converts a first color light into a third color light, and a third light controlling part CCP3 that transmits a first color light.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the light emitting device ED. For example, the first quantum dot QD1 may be a red quantum dot, and the second quantum dot QD2 may be a green quantum dot. For the quantum dots QD1 and QD2, the same description as that above may be applied.

In addition, the light controlling layer CCL may further include a scatterer SP (e.g., a light scatterer SP). The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 that disperse the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of various suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking or reducing the penetration of moisture and/or oxygen (hereinafter, may be referred to as “humidity/oxygen”). The barrier layer BFL1 may be on the light controlling parts CCP1, CCP2 and CCP3 to block or reduce the exposure of the light controlling parts CCP1, CCP2 and CCP3 to humidity/oxygen. In some embodiments, the barrier layer BFL1 may cover the light controlling parts CCP1, CCP2 and CCP3. In addition, the barrier layer BFL2 may be provided between the light controlling parts CCP1, CCP2 and CCP3 and a color filter layer CFL.

The barrier layers BFL1 and BFL2 may include at least one inorganic layer. In some embodiments, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride or a metal thin film securing light transmittance. In some embodiments, the barrier layers BFL1 and BFL2 may further include an organic layer. The barrier layers BFL1 and BFL2 may be composed of a single layer of multiple layers.

In the display apparatus DD of an embodiment, the color filter layer CFL may be on the light controlling layer CCL. For example, the color filter layer CFL may be directly on the light controlling layer CCL. In this case, the barrier layer BFL2 may be omitted.

The color filter layer CFL may include a light blocking part BM and filters CF1, CF2 and CF3. The color filter layer CFL may include a first filter CF1 that transmits a second color light, a second filter CF2 that transmits a third color light, and a third filter CF3 that transmits a first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. Embodiments of the present disclosure are not limited thereto, however, and the third filter CF3 may not include the pigment and/or dye. The third filter CF3 may include a polymer photosensitive resin and not include a pigment and/or dye. The third filter CF3 may be transparent. The third filter CF3 may be formed using a transparent photosensitive resin.

In addition, in an embodiment, the first filter CF1 and the second filter CF2 may be yellow filters. The first filter CF1 and the second filter CF2 may be provided in one body without distinction.

The light blocking part BM may be a black matrix. The light blocking part BM may be formed by including an organic light blocking material or an inorganic light blocking material including a black pigment and/or black dye. The light blocking part BM may prevent or reduce light leakage phenomenon and divide the boundaries among adjacent filters CF1, CF2 and CF3. In addition, in an embodiment, the light blocking part BM may be formed as a blue filter.

The first to third filters CF1, CF2 and CF3 may respectively correspond to a red luminous area PXA-R, green luminous area PXA-G, and blue luminous area PXA-B.

A base substrate BL may be on the color filter layer CFL. The base substrate BL may be a member providing a base surface that the color filter layer CFL, the light controlling layer CCL, etc. are on. The base substrate BL may be a glass substrate, a metal substrate, a plastic substrate, etc. However, an embodiment of the present disclosure is not limited thereto, and the base substrate BL may be an inorganic layer, an organic layer or a composite material layer. In addition, different from the drawing, the base substrate BL may be omitted in an embodiment.

FIG. 10 is a cross-sectional view showing a portion of the display apparatus according to an embodiment. In a display apparatus DD-TD of an embodiment, the light emitting device ED-BT may include multiple light emitting structures OL-B1, OL-B2 and OL-B3. The light emitting device ED-BT may include the first electrode EL1 facing the second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 9), and a hole transport region HTR and an electron transport region ETR with the emission layer EML (FIG. 9) therebetween.

In some embodiments, the light emitting device ED-BT included in the display apparatus DD-TD of an embodiment may be a light emitting device of a tandem structure including multiple emission layers.

In an embodiment shown in FIG. 10, light emitted from the light emitting structures OL-B1, OL-B2 and OL-B3 may all be blue light. However, an embodiment of the present disclosure is not limited thereto, and the wavelength regions of light emitted from the light emitting structures OL-B1, OL-B2 and OL-B3 may be different from each other. For example, the light emitting device ED-BT including the multiple light emitting structures OL-B1, OL-B2 and OL-B3 emitting light in different wavelength regions may emit white light.

A charge generating layer CGL may be between neighboring light emitting structures OL-B1, OL-B2 and OL-B3. The charge generating layer CGL may include a p-type charge generating layer and/or an n-type charge generating layer.

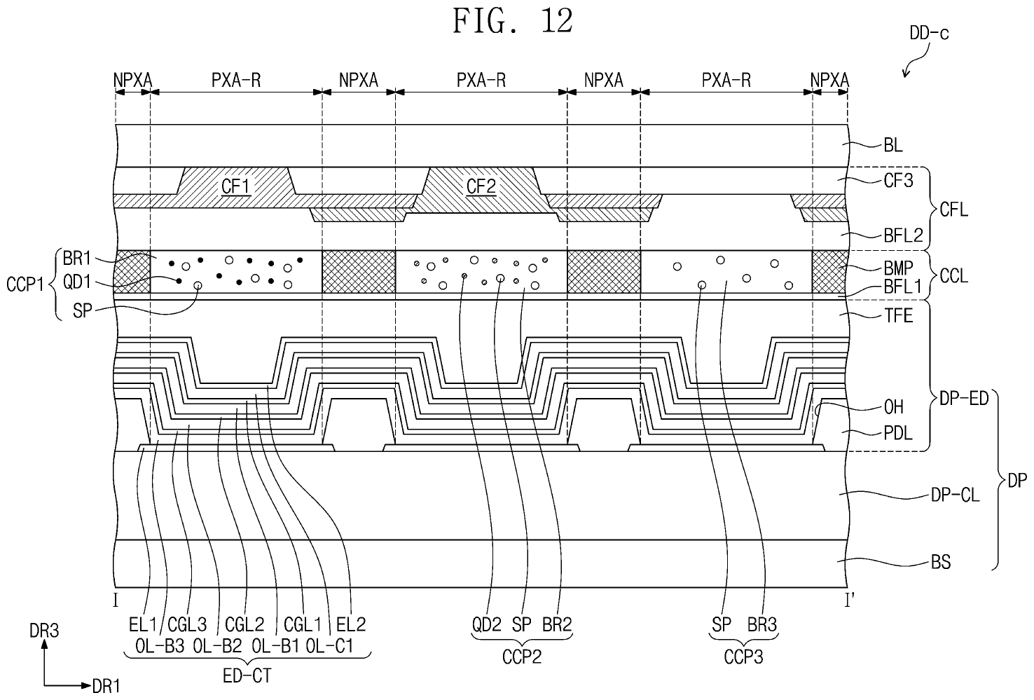

Referring to FIG. 11, a display apparatus DD-b according to an embodiment may include light emitting devices ED-1, ED-2 and ED-3, formed by stacking two emission layers. Compared to the display apparatus DD of an embodiment, shown in FIG. 9, an embodiment shown in FIG. 11 is different in that first to third light emitting devices ED-1, ED-2 and ED-3 include two emission layers stacked in a thickness direction, each. In the first to third light emitting devices ED-1, ED-2 and ED-3, two emission layers may emit light in the same (e.g., substantially the same) wavelength region.

The first light emitting device ED-1 may include a first red emission layer EML-R1 and a second red emission layer EML-R2. The second light emitting device ED-2 may include a first green emission layer EML-G1 and a second green emission layer EML-G2. In addition, the third light emitting device ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and an emission auxiliary part OG may be between the first blue emission layer EML-B1 and the second blue emission layer EML-B2.

The emission auxiliary part OG may include a single layer or a multilayer. The emission auxiliary part OG may include a charge generating layer. In some embodiments, the emission auxiliary part OG may include an electron transport region, a charge generating layer, and a hole transport region stacked in order. The emission auxiliary part OG may be provided as a common layer in all of the first to third light emitting devices ED-1, ED-2 and ED-3. However, an embodiment of the present disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be between the electron transport region ETR and the emission auxiliary part OG. The second red emission layer EML-R2, the second green emission layer EML-G2 and the second blue emission layer EML-B2 may be between the emission auxiliary part OG and the hole transport region HTR.

In some embodiments, the first light emitting device ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting device ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting device ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be on a display element layer DP-ED. The optical auxiliary layer PL may include a polarization layer. The optical auxiliary layer PL may be on a display panel DP and may control reflected light at the display panel DP by external light. Different from the drawings, the optical auxiliary layer PL may be omitted from the display apparatus according to an embodiment.

Different from FIG. 9 and FIG. 10, a display apparatus DD-c in FIG. 12 is shown to include four light emitting structures OL-B1, OL-B2, OL-B3 and OL-C1. A light emitting device ED-CT may include the first electrode EU facing the second electrode EL2, and first to fourth light emitting structures OL-B1, OL-B2, OL-B3 and OL-C1 stacked in order in a thickness direction between the first electrode EL1 and the second electrode EL2. Charge generating layers CGL1, CGL2 and CGL3 may be respectively between the first to fourth light emitting structures OL-B1, OL-B2, OL-B3 and OL-C1. Among the four light emitting structures, the first to third light emitting structures OL-B1, OL-B2 and OL-B3 may emit blue light, and the fourth light emitting structure OL-C1 may emit green light. However, an embodiment of the present disclosure is not limited thereto, and the first to fourth light emitting structures OL-B1, OL-B2, OL-B3 and OL-C1 may emit different wavelengths of light.

Hereinafter, the compound according to an embodiment and the light emitting device of an embodiment of the present disclosure will be particularly explained referring to examples and comparative examples. In addition, the embodiments below are only examples to assist the understanding of the subject matter of the present disclosure, and the scope of the present disclosure is not limited thereto.

### EXAMPLES

### 1. Synthesis of Amine Compounds

First, the synthetic method of an amine compound according to an embodiment will be explained in more detail with respect to the synthetic methods of Compounds 1, 6, 15, 28, 36, 43, 49, 145, 707, 803, 808, 851, 865, 868, 869, 872, 873, 876, 877, 880, 881, 884, 995, 996, 1024, 1035, 1044, and 1059. In addition, the synthetic methods of the amine compounds explained hereinafter are examples, and the synthetic method of the amine compound according to an embodiment of the present disclosure is not limited to the embodiments below.

### (1) Synthesis of Compound 15

Amine Compound 15 according to an embodiment may be synthesized, for example, by the reaction below.

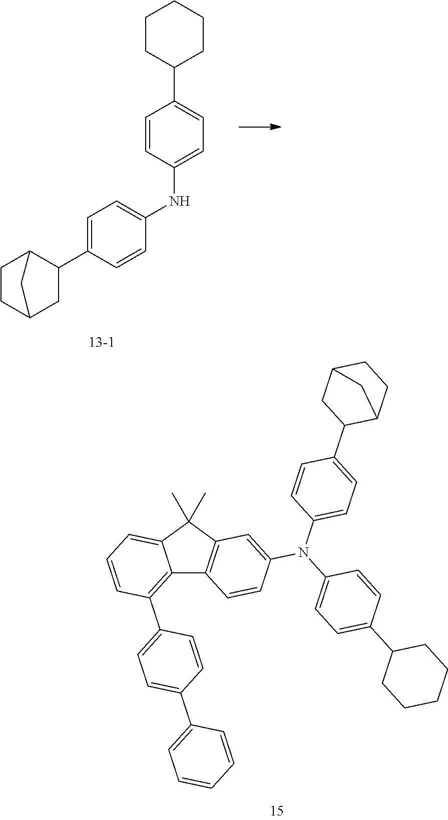 [SMILES: c1cc(C2CCCCC2)ccc1Nc1ccc(C2CC3CCC2C3)cc1 ; CC1(C)c2cc(N(c3ccc(C4CCCCC4)cc3)c3ccc(C4CC5CCC4C5)cc3)ccc2-c2c(-c3ccc(-c4ccccc4)cc3)cccc21]

### Synthesis of Compound 15

Intermediate 13-1 (1 eq, 10 mmol), 5-([1,1′-biphenyl]-4-yl)-2-bromo-9,9-dimethyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.83 g of Compound 15 (yield: 70%, ≥purity 99.9%).

### (2) Synthesis of Compound 28

Amine Compound 28 according to an embodiment may be synthesized, for example, by the reaction below.

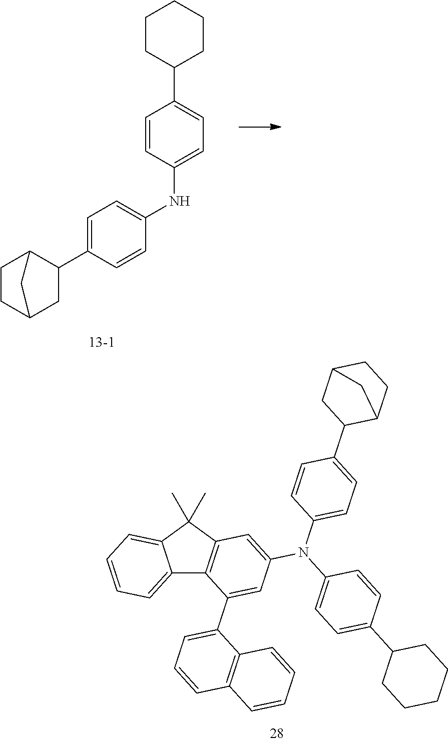 [SMILES: c1cc(C2CCCCC2)ccc1Nc1ccc(C2CC3CCC2C3)cc1 ; CC1(C)c2ccccc2-c2c(-c3cccc4ccccc34)cc(N(c3ccc(C4CCCCC4)cc3)c3ccc(C4CC5CCC4C5)cc3)cc21]

### Synthesis of Compound 28

Intermediate 13-1 (1 eq, 10 mmol), 2-bromo-9,9-dimethyl-4-(naphthalen-1-yl)-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.35 g of Compound 28 (yield: 66%, ≥purity 99.9%).

### (3) Synthesis of Compound 36

Amine Compound 36 according to an embodiment may be synthesized, for example, by the reaction below.

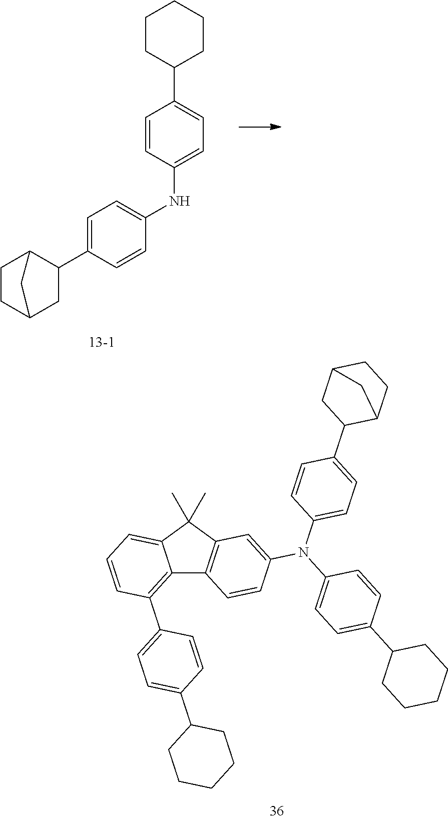 [SMILES: c1cc(C2CCCCC2)ccc1Nc1ccc(C2CC3CCC2C3)cc1 ; CC1(C)c2cc(N(c3ccc(C4CCCCC4)cc3)c3ccc(C4CC5CCC4C5)cc3)ccc2-c2c(-c3ccc(C4CCCCC4)cc3)cccc21]

### Synthesis of Compound 36

Intermediate 13-1 (1 eq, 10 mmol), 2-bromo-5-(4-cyclohexylphenyl)-9,9-dimethyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.08 g of Compound 36 (yield: 73%, ≥purity 99.9%).

### (4) Synthesis of Compound 43

Amine Compound 43 according to an embodiment may be synthesized, for example, by the reaction below.

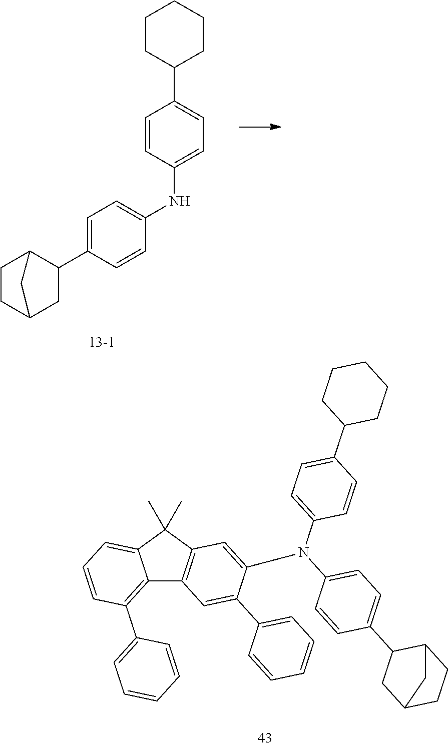 [SMILES: c1cc(C2CCCCC2)ccc1Nc1ccc(C2CC3CCC2C3)cc1 ; CC1(C)c2cc(N(c3ccc(C4CCCCC4)cc3)c3ccc(C4CC5CCC4C5)cc3)c(-c3ccccc3)cc2-c2c(-c3ccccc3)cccc21]

### Synthesis of Compound 43

Intermediate 13-1 (1 eq, 10 mmol), 2-bromo-9,9-dimethyl-3,5-diphenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.75 g of Compound 43 (yield: 69%, purity ≥99.9%).

### (5) Synthesis of Compound 49

Amine Compound 49 according to an embodiment may be synthesized, for example, by the reaction below.

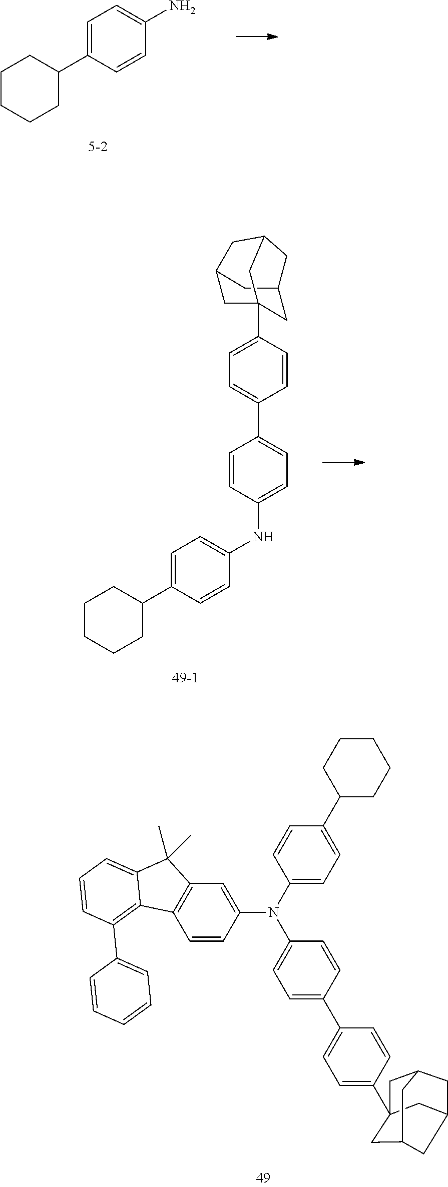 [SMILES: Nc1ccc(C2CCCCC2)cc1 ; c1cc(-c2ccc(C34CC5CC(CC(C5)C3)C4)cc2)ccc1Nc1ccc(C2CCCCC2)cc1 ; CC1(C)c2cc(N(c3ccc(-c4ccc(C56CC7CC(CC(C7)C5)C6)cc4)cc3)c3ccc(C4CCCCC4)cc3)ccc2-c2c(-c3ccccc3)cccc21]

### Synthesis of Intermediate 49-1

Intermediate 5-2 (1.2 eq, 22 mmol), (1r,3R,5S)-1-(4′-bromo-[1,1′-biphenyl]-4-yl)adamantane (1 eq, 20 mmol), Pd₂(dba)₃ (0.03 eq, 0.6 mmol), t-BuONa (3 eq, 60 mmol), t-Bu₃P (0.06 eq, 1.2 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 8 g of Intermediate 49-1 (yield: 87%, purity ≥99.9%).

### Synthesis of Compound 49

Intermediate 49-1 (1 eq, 10 mmol), 2-bromo-9,9-dimethyl-5-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 300 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.61 g of Compound 49 (yield: 77%, ≥purity 99.9%).

### (6) Synthesis of Compound 145

Amine Compound 145 according to an embodiment may be synthesized, for example, by the reaction below.

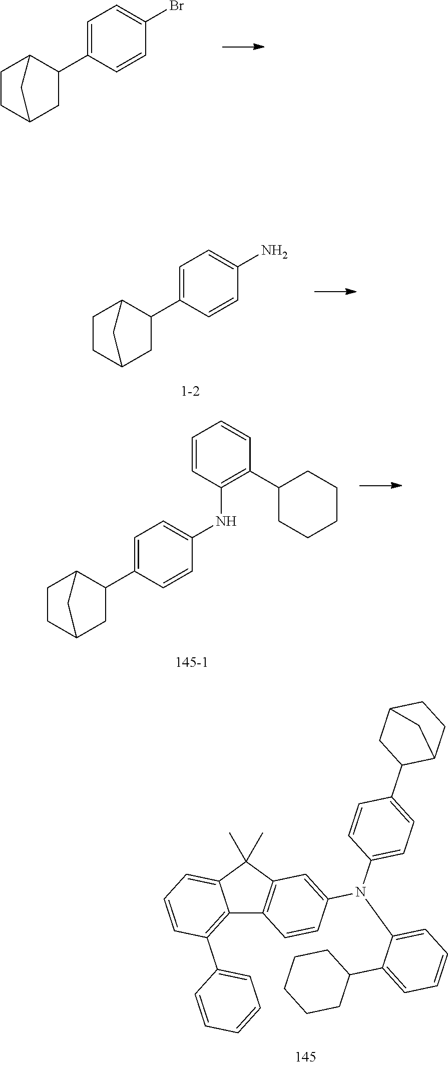 [SMILES: Brc1ccc(C2CC3CCC2C3)cc1 ; Nc1ccc(C2CC3CCC2C3)cc1 ; c1ccc(C2CCCCC2)c(Nc2ccc(C3CC4CCC3C4)cc2)c1 ; CC1(C)c2cc(N(c3ccc(C4CC5CCC4C5)cc3)c3ccccc3C3CCCCC3)ccc2-c2c(-c3ccccc3)cccc21]

### Synthesis of Intermediate 1-2

2-(4-Bromophenyl)bicyclo[2.2.1]heptane (1 eq, 100 mmol), CuI (3 eq, 300 mmol), ammonia solution (1 eq, 100 mmol), 100 ml of DMF were put in a seal tube, followed by stirring at about 120° C. for about 6 hours. After finishing the reaction, the reaction product was worked up with H₂O and ether, and an organic layer was separated by column chromatography (eluent: methylene chloride/hexane=1/3 by volume ratio) to obtain 9.3 g of Intermediate 1-2 (yield: 50%, purity ≥99.9%).

### Synthesis of Intermediate 145-1

Intermediate 1-2 (1.2 eq, 22 mmol), 1-bromo-2-cyclohexylbenzene (1 eq, 20 mmol), Pd₂(dba)₃ (0.03 eq, 0.6 mmol), t-BuONa (3 eq, 60 mmol), t-Bu₃P (0.06 eq, 1.2 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.48 g of Intermediate 145-1 (yield: 65%, purity ≥99.9%).

### Synthesis of Compound 145

Intermediate 145-1 (1 eq, 10 mmol), 2-bromo-9,9-dimethyl-5-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.47 g of Compound 145 (yield: 73%, purity ≥99.9%).

### (7) Synthesis of Compound 707

Amine Compound 707 according to an embodiment may be synthesized, for example, by the reaction below.

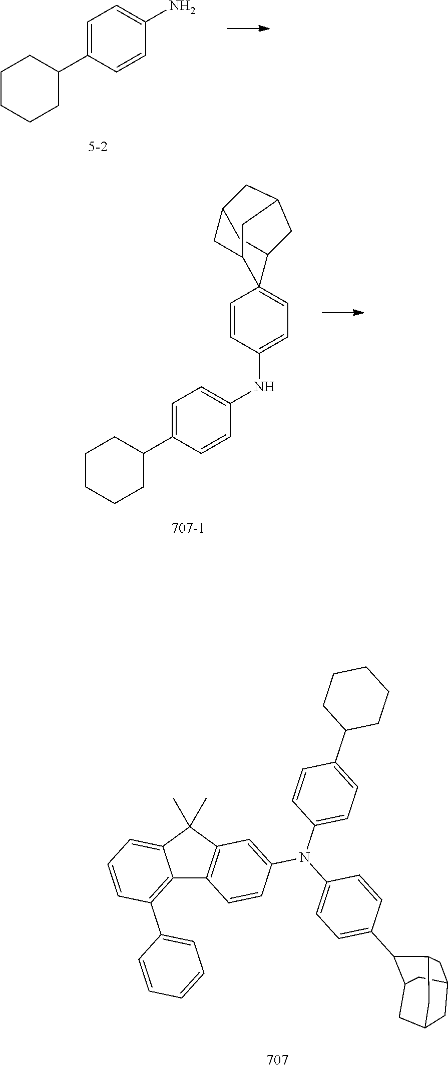

### Synthesis of Intermediate 707-1

Intermediate 5-2 (1.2 eq, 22 mmol), (1R,2r,3S,5r)-2-(4-bromophenyl)adamantane (1 eq, 20 mmol), Pd₂(dba)₃ (0.03 eq, 0.6 mmol), t-BuONa (3 eq, 60 mmol), t-Bu₃P (0.06 eq, 1.2 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 6.7 g of Intermediate 707-1 (yield: 87%, purity 99.9%).

### Synthesis of Compound 707

Intermediate 707-1 (1 eq, 10 mmol), 2-bromo-9,9-dimethyl-5-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 300 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.22 g of Compound 707 (yield: 80%, purity ≥99.9%).

### (8) Synthesis of Compound 851

Amine Compound 851 according to an embodiment may be synthesized, for example, by the reaction below.

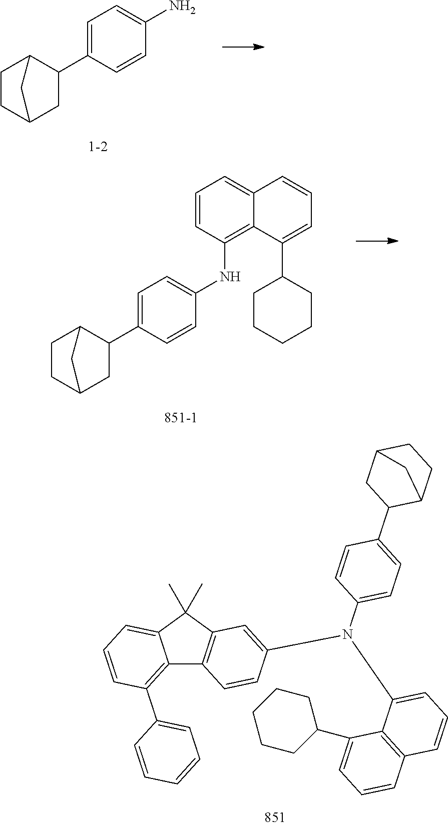 [SMILES: Nc1ccc(C2CC3CCC2C3)cc1 ; c1cc(Nc2ccc(C3CC4CCC3C4)cc2)c2c(C3CCCCC3)cccc2c1 ; CC1(C)c2cc(N(c3ccc(C4CC5CCC4C5)cc3)c3cccc4cccc(C5CCCCC5)c34)ccc2-c2c(-c3ccccc3)cccc21]

### Synthesis of Intermediate 851-1

Intermediate 1-2 (1.2 eq, 22 mmol), 1-bromo-8-cyclohexylnaphthalene (1 eq, 20 mmol), Pd₂(dba)₃ (0.03 eq, 0.6 mmol), t-BuONa (3 eq, 60 mmol), t-Bu₃P (0.06 eq, 1.2 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.46 g of Intermediate 851-1 (yield: 62%, purity ≥99.9%).

### Synthesis of Compound 851

Intermediate 851-1 (1 eq, 10 mmol), 2-bromo-9,9-dimethyl-5-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 4.11 g of Compound 851 (yield: 62%, purity ≥99.9%).

### (9) Synthesis of Compound 865

Amine Compound 865 according to an embodiment may be synthesized, for example, by the reaction below.

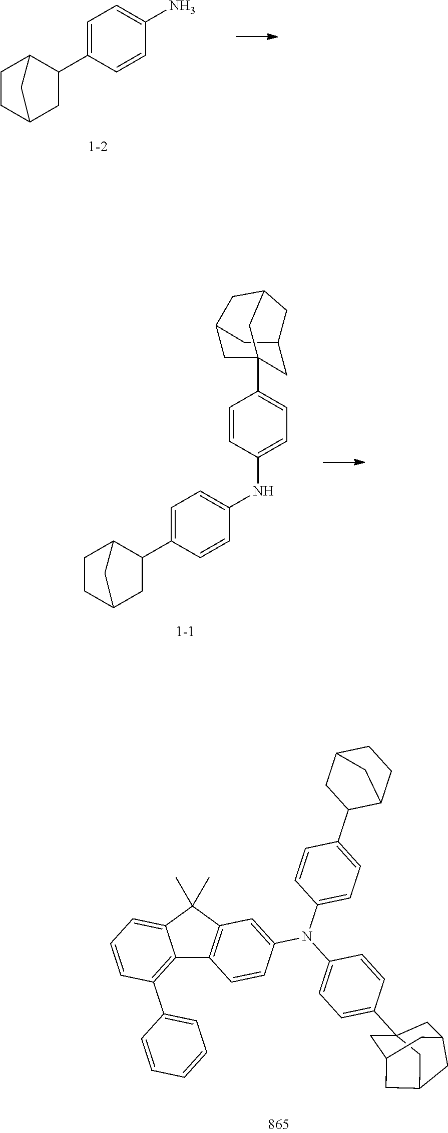 [SMILES: Nc1ccc(C2CC3CCC2C3)cc1 ; c1cc(C2CC3CCC2C3)ccc1Nc1ccc(C23CC4CC(CC(C4)C2)C3)cc1 ; CC1(C)c2cc(N(c3ccc(C4CC5CCC4C5)cc3)c3ccc(C45CC6CC(CC(C6)C4)C5)cc3)ccc2-c2c(-c3ccccc3)cccc21]

### Synthesis of Intermediate 1-1

Intermediate 1-2 (1.2 eq, 50 mmol), (1r,3R,5S)-1-(4-bromophenyl)adamantane (1 eq, 44 mmol), Pd₂(dba)₃ (0.03 eq, 1.5 mmol), t-BuONa (3 eq, 150 mmol), t-Bu₃P (0.06 eq, 3 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 16 g of Intermediate 1-1 (yield: 80%, purity ≥99.9%).

### Synthesis of Compound 865

Intermediate 1-1 (1 eq, 40 mmol), 2-bromo-9,9-dimethyl-5-phenyl-9H-fluorene (1.1 eq, 44 mmol), Pd₂(dba)₃ (0.03 eq, 1.2 mmol), t-BuONa (3 eq, 120 mmol), t-Bu₃P (0.06 eq, 2.4 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 20 g of Compound 865 (yield: 75%, purity ≥99.9%).

### (10) Synthesis of Compound 868

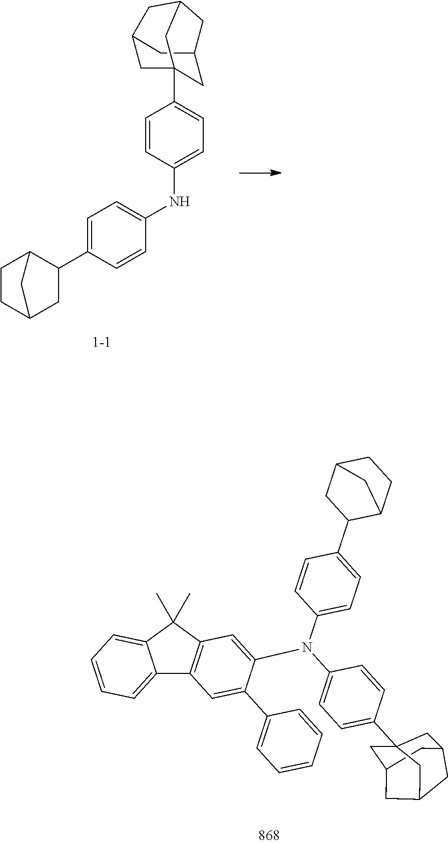 [SMILES: c1cc(C2CC3CCC2C3)ccc1Nc1ccc(C23CC4CC(CC(C4)C2)C3)cc1 ; CC1(C)c2ccccc2-c2cc(-c3ccccc3)c(N(c3ccc(C4CC5CCC4C5)cc3)c3ccc(C45CC6CC(CC(C6)C4)C5)cc3)cc21]

### Synthesis of Compound 868

Intermediate 1-1 (1 eq, 40 mmol), 2-bromo-9,9-dimethyl-3-phenyl-9H-fluorene (1.1 eq, 44 mmol), Pd₂(dba)₃ (0.03 eq, 1.2 mmol), t-BuONa (3 eq, 120 mmol), t-Bu₃P (0.06 eq, 2.4 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 18.6 g of Compound 868 (yield: 70%, purity ≥99.9%).

### (11) Synthesis of Compound 803

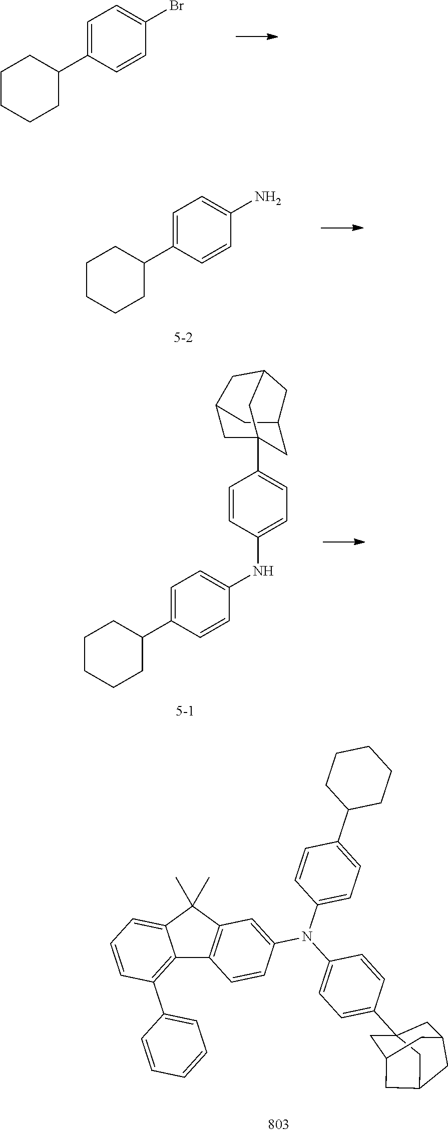 [SMILES: Brc1ccc(C2CCCCC2)cc1 ; Nc1ccc(C2CCCCC2)cc1 ; c1cc(C2CCCCC2)ccc1Nc1ccc(C23CC4CC(CC(C4)C2)C3)cc1 ; CC1(C)c2cc(N(c3ccc(C4CCCCC4)cc3)c3ccc(C45CC6CC(CC(C6)C4)C5)cc3)ccc2-c2c(-c3ccccc3)cccc21]

### Synthesis of Intermediate 5-2

1-Bromo-4-cyclohexylbenzene (1 eq, 100 mmol), CuI (3 eq, 300 mmol), ammonia solution (1 eq, 100 mmol), 100 ml of DMF were put in a seal tube, followed by stirring at about 120° C. for about 6 hours. After finishing the reaction, the reaction product was worked up with H₂O and ether, and an organic layer was separated by column chromatography (eluent: methylene chloride/hexane=1/3 by volume ratio) to obtain 10.5 g of Intermediate 5-2 (yield: 60%, purity ≥99.9%).

### Synthesis of Intermediate 5-1

Intermediate 5-2 (1.2 eq, 60 mmol), (1r,3R,5S)-1-(4-bromophenyl)adamantane (1 eq, 50 mmol), Pd₂(dba)₃ (0.03 eq, 1.5 mmol), t-BuONa (3 eq, 150 mmol), t-Bu₃P (0.06 eq, 3 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 20 g of Intermediate 5-1 (yield: 87%, purity ≥99.9%).

### Synthesis of Compound 803

Intermediate 5-1 (1 eq, 52 mmol), 2-bromo-9,9-dimethyl-5-phenyl-9H-fluorene (1.1 eq, 57.2 mmol), Pd₂(dba)₃ (0.03 eq, 1.56 mmol), t-BuONa (3 eq, 156 mmol), t-Bu₃P (0.06 eq, 3.12 mmol), and 300 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 23.7 g of Compound 803 (yield: 70%, purity 99.9%).

### (12) Synthesis of Compound 808

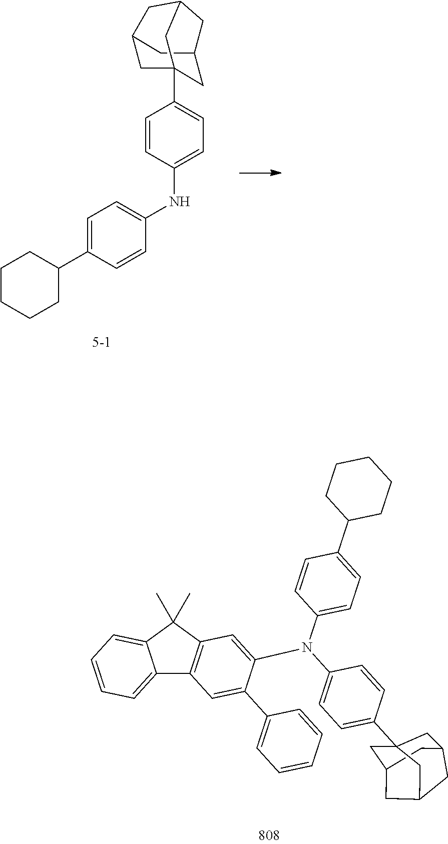 [SMILES: c1cc(C2CCCCC2)ccc1Nc1ccc(C23CC4CC(CC(C4)C2)C3)cc1 ; CC1(C)c2ccccc2-c2cc(-c3ccccc3)c(N(c3ccc(C4CCCCC4)cc3)c3ccc(C45CC6CC(CC(C6)C4)C5)cc3)cc21]

### Synthesis of Compound 808

Intermediate 5-1 (1 eq, 10 mmol), 2-bromo-9,9-dimethyl-3-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 6 g of Compound 808 (yield: 90%, purity ≥99.9%).

### (13) Synthesis of Compound 869

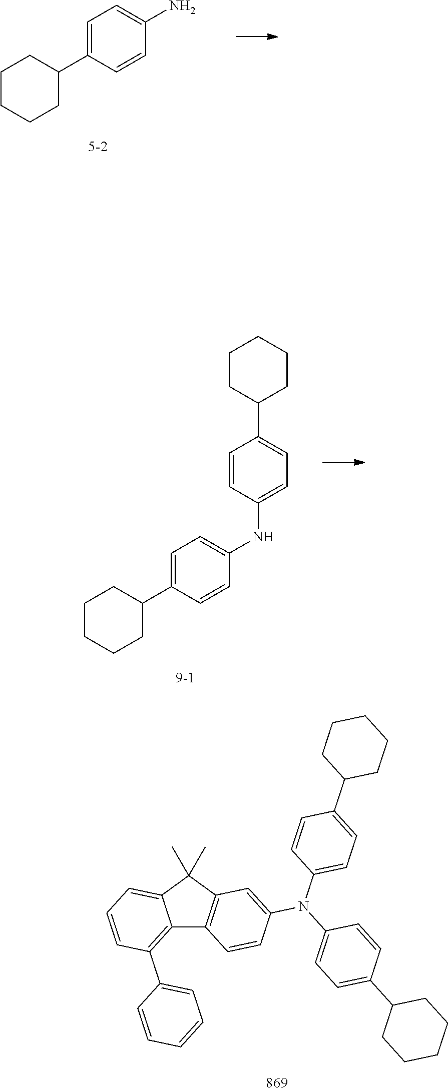 [SMILES: Nc1ccc(C2CCCCC2)cc1 ; c1cc(C2CCCCC2)ccc1Nc1ccc(C2CCCCC2)cc1 ; CC1(C)c2cc(N(c3ccc(C4CCCCC4)cc3)c3ccc(C4CCCCC4)cc3)ccc2-c2c(-c3ccccc3)cccc21]

### Synthesis of Intermediate 9-1

Intermediate 5-2 (1.2 eq, 30 mmol), (1r,3R,5S)-1-(4-bromophenyl)adamantane (1 eq, 25 mmol), Pd₂(dba)₃ (0.03 eq, 0.75 mmol), t-BuONa (3 eq, 75 mmol), t-Bu₃P (0.06 eq, 1.5 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 6.8 g of Intermediate 9-1 (yield: 82%, purity ≥99.9%).

### Synthesis of Compound 869

Intermediate 9-1 (1 eq, 20.5 mmol), 2-bromo-9,9-dimethyl-5-phenyl-9H-fluorene (1.1 eq, 22.55 mmol), Pd₂(dba)₃ (0.03 eq, 0.6 mmol), t-BuONa (3 eq, 61.5 mmol), t-Bu₃P (0.06 eq, 1.23 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 12 g of Compound 869 (yield: 88%, purity ≥99.9%).

### (14) Synthesis of Compound 872

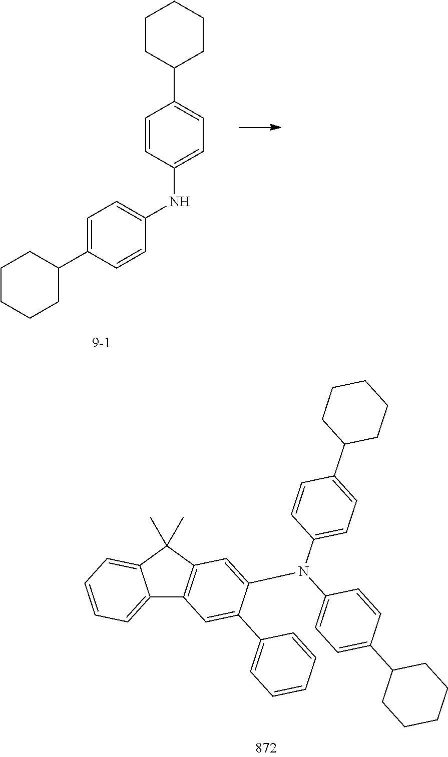 [SMILES: c1cc(C2CCCCC2)ccc1Nc1ccc(C2CCCCC2)cc1 ; CC1(C)c2ccccc2-c2cc(-c3ccccc3)c(N(c3ccc(C4CCCCC4)cc3)c3ccc(C4CCCCC4)cc3)cc21]

### Synthesis of Compound 872

Intermediate 9-1 (1 eq, 10 mmol), 2-bromo-9,9-dimethyl-3-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.3 g of Compound 872 (yield: 80%, purity ≥99.9%).

### (15) Synthesis of Compound 1

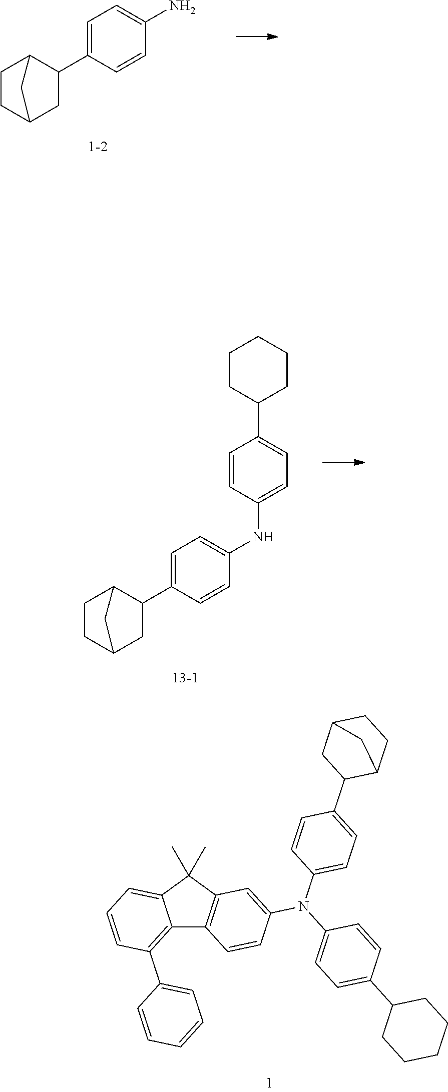 [SMILES: Nc1ccc(C2CC3CCC2C3)cc1 ; c1cc(C2CCCCC2)ccc1Nc1ccc(C2CC3CCC2C3)cc1 ; CC1(C)c2cc(N(c3ccc(C4CCCCC4)cc3)c3ccc(C4CC5CCC4C5)cc3)ccc2-c2c(-c3ccccc3)cccc21]

### Synthesis of Intermediate 13-1

Intermediate 1-2 (1.2 eq, 50 mmol), (1r,3R,5S)-1-(4-bromophenyl)adamantane (1 eq, 44 mmol), Pd₂(dba)₃ (0.03 eq, 1.5 mmol), t-BuONa (3 eq, 150 mmol), t-Bu₃P (0.06 eq, 3 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 13.8 g of Intermediate 13-1 (yield: 80%, purity 99.9%).

### Synthesis of Compound 1

Intermediate 13-1 (1 eq, 40 mmol), 2-bromo-9,9-dimethyl-5-phenyl-9H-fluorene (1.1 eq, 44 mmol), Pd₂(dba)₃ (0.03 eq, 1.2 mmol), t-BuONa (3 eq, 120 mmol), t-Bu₃P (0.06 eq, 2.4 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 17.4 g of Compound 1 (yield: 71%, purity ≥99.9%).

### (16) Synthesis of Compound 6

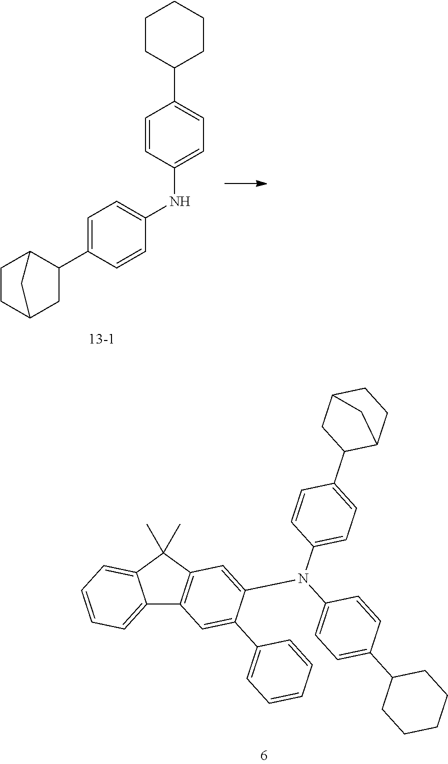 [SMILES: c1cc(C2CCCCC2)ccc1Nc1ccc(C2CC3CCC2C3)cc1 ; CC1(C)c2ccccc2-c2cc(-c3ccccc3)c(N(c3ccc(C4CCCCC4)cc3)c3ccc(C4CC5CCC4C5)cc3)cc21]

### Synthesis of Compound 6

Intermediate 13-1 (1 eq, 10 mmol), 2-bromo-9,9-dimethyl-3-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.3 g of Compound 6 (yield: 85%, purity ≥99.9%).

### (17) Synthesis of Compound 873

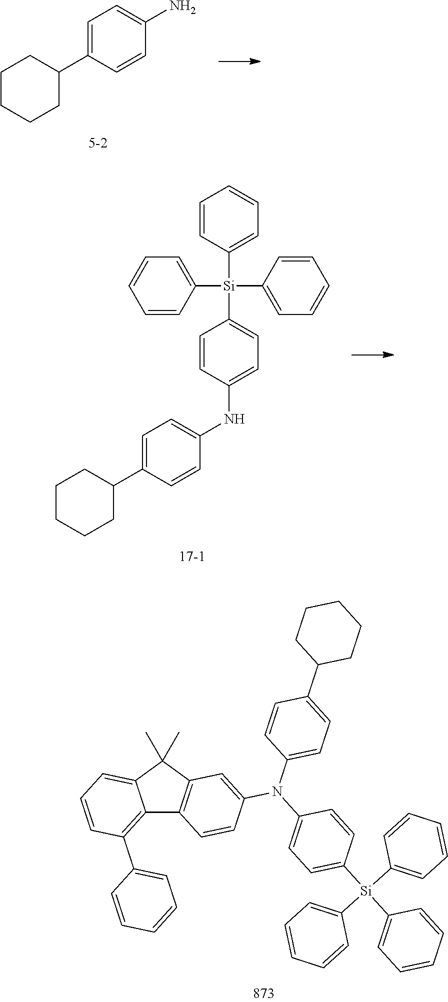 [SMILES: Nc1ccc(C2CCCCC2)cc1 ; c1ccc([Si](c2ccccc2)(c2ccccc2)c2ccc(Nc3ccc(C4CCCCC4)cc3)cc2)cc1 ; CC1(C)c2cc(N(c3ccc(C4CCCCC4)cc3)c3ccc([Si](c4ccccc4)(c4ccccc4)c4ccccc4)cc3)ccc2-c2c(-c3ccccc3)cccc21]

### Synthesis of Intermediate 17-1

Intermediate 5-2 (1.2 eq, 30 mmol), (4-bromophenyl)triphenylsilane (1 eq, 25 mmol), Pd₂(dba)₃ (0.03 eq, 0.75 mmol), t-BuONa (3 eq, 75 mmol), t-Bu₃P (0.06 eq, 1.5 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 9.8 g of Intermediate 17-1 (yield: 77%, purity ≥99.9%).

### Synthesis of Compound 873

Intermediate 17-1 (1 eq, 19 mmol), 2-bromo-9,9-dimethyl-5-phenyl-9H-fluorene (1.1 eq, 21 mmol), Pd₂(dba)₃ (0.03 eq, 0.57 mmol), t-BuONa (3 eq, 57 mmol), t-Bu₃P (0.06 eq, 1.14 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 9.6 g of Compound 873 (yield: 65%, purity ≥99.9%).

### (18) Synthesis of Compound 876

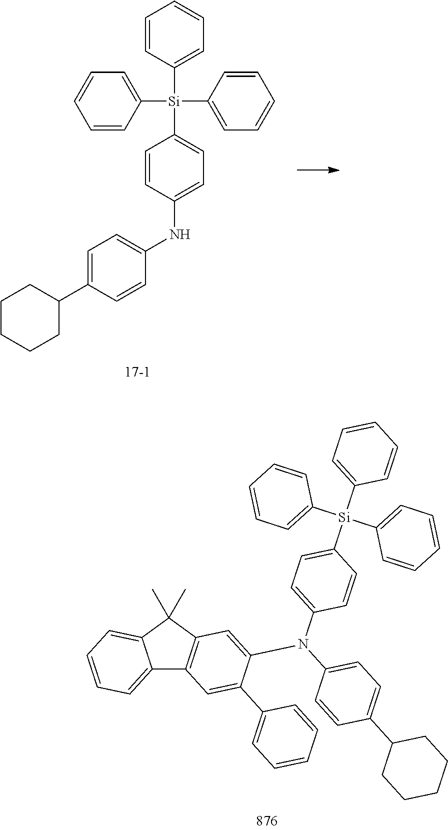 [SMILES: c1ccc([Si](c2ccccc2)(c2ccccc2)c2ccc(Nc3ccc(C4CCCCC4)cc3)cc2)cc1 ; CC1(C)c2ccccc2-c2cc(-c3ccccc3)c(N(c3ccc(C4CCCCC4)cc3)c3ccc([Si](c4ccccc4)(c4ccccc4)c4ccccc4)cc3)cc21]

### (Synthesis of Compound 876)

Intermediate 17-1 (1 eq, 10 mmol), 2-bromo-9,9-dimethyl-3-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.3 g of Compound 876 (yield: 80%, purity ≥99.9%).

### (19) Synthesis of Compound 877

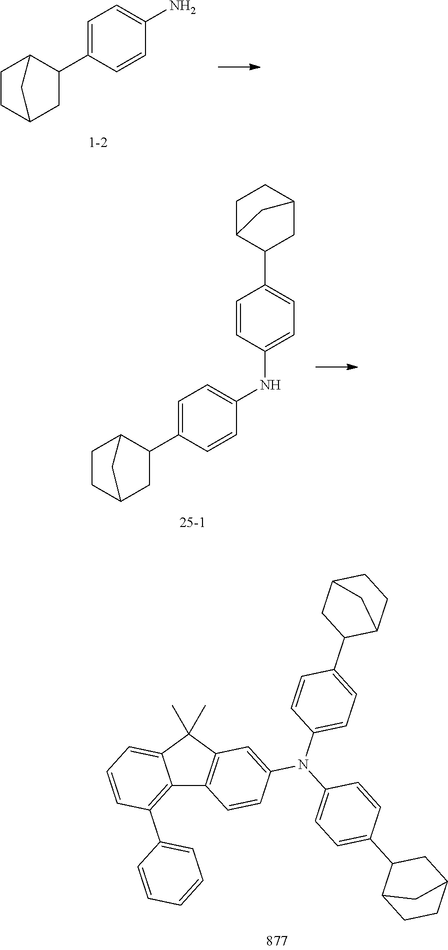 [SMILES: Nc1ccc(C2CC3CCC2C3)cc1 ; c1cc(C2CC3CCC2C3)ccc1Nc1ccc(C2CC3CCC2C3)cc1 ; CC1(C)c2cc(N(c3ccc(C4CC5CCC4C5)cc3)c3ccc(C4CC5CCC4C5)cc3)ccc2-c2c(-c3ccccc3)cccc21]

### (Synthesis of Intermediate 25-1)

Intermediate 1-2 (1.2 eq, 30 mmol), 2-(4-bromophenyl)bicyclo[2.2.1]heptane (1 eq, 25 mmol), Pd₂(dba)₃ (0.03 eq, 0.75 mmol), t-BuONa (3 eq, 75 mmol), t-Bu₃P (0.06 eq, 1.5 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 6.24 g of Intermediate 25-1 (yield: 70%, purity 99.9%).

### (Synthesis of Compound 877)

Intermediate 25-1 (1 eq, 17.5 mmol), 2-bromo-9,9-dimethyl-5-phenyl-9H-fluorene (1.1 eq, 19 mmol), Pd₂(dba)₃ (0.03 eq, 0.53 mmol), t-BuONa (3 eq, 30 mmol), t-Bu₃P (0.06 eq, 1.05 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 8.2 g of Compound 877 (yield: 75%, purity ≥99.9%).

### (20) Synthesis of Compound 880

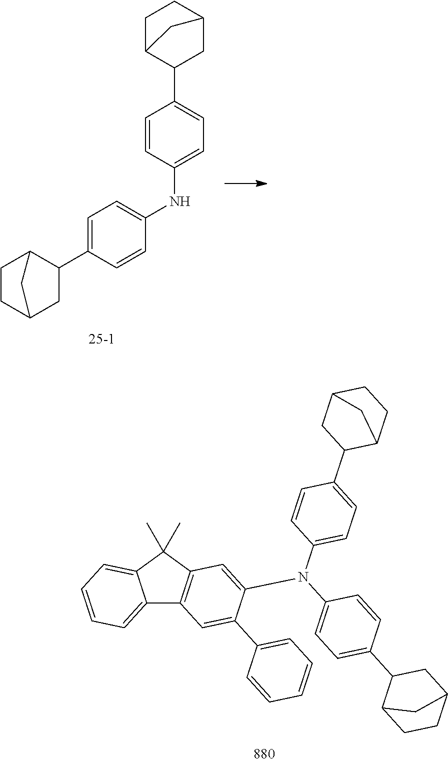 [SMILES: c1cc(C2CC3CCC2C3)ccc1Nc1ccc(C2CC3CCC2C3)cc1 ; CC1(C)c2ccccc2-c2cc(-c3ccccc3)c(N(c3ccc(C4CC5CCC4C5)cc3)c3ccc(C4CC5CCC4C5)cc3)cc21]

### (Synthesis of Compound 880)

Intermediate 25-1 (1 eq, 10 mmol), 2-bromo-9,9-dimethyl-3-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.6 g of Compound 880 (yield: 90%, purity ≥99.9%).

### (21) Synthesis of Compound 881

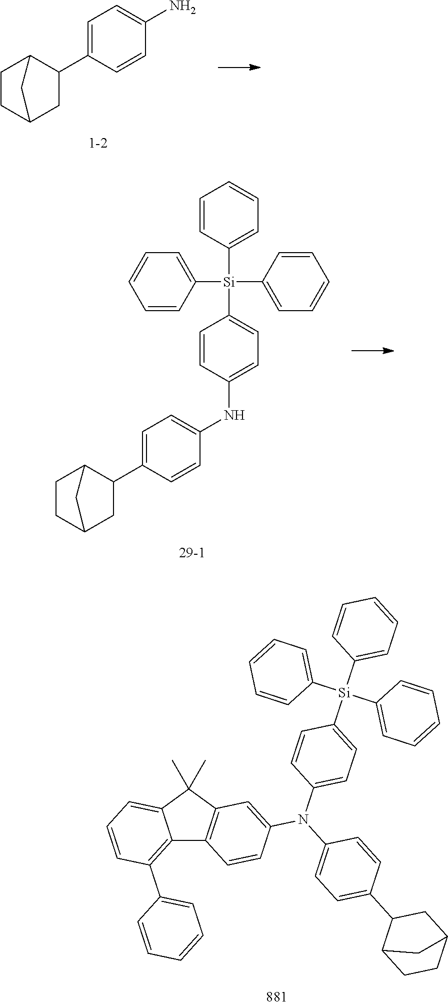 [SMILES: Nc1ccc(C2CC3CCC2C3)cc1 ; c1ccc([Si](c2ccccc2)(c2ccccc2)c2ccc(Nc3ccc(C4CC5CCC4C5)cc3)cc2)cc1 ; CC1(C)c2cc(N(c3ccc(C4CC5CCC4C5)cc3)c3ccc([Si](c4ccccc4)(c4ccccc4)c4ccccc4)cc3)ccc2-c2c(-c3ccccc3)cccc21]

### Synthesis of Intermediate 29-1

Intermediate 1-2 (1.2 eq, 30 mmol), (4-bromophenyl)triphenylsilane (1 eq, 25 mmol), Pd₂(dba)₃ (0.03 eq, 0.75 mmol), t-BuONa (3 eq, 75 mmol), t-Bu₃P (0.06 eq, 1.5 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 9.8 g of Intermediate 29-1 (yield: 77%, purity ≥99.9%).

### Synthesis of Compound 881

Intermediate 29-1 (1 eq, 19 mmol), 2-bromo-9,9-dimethyl-5-phenyl-9H-fluorene (1.1 eq, 21 mmol), Pd₂(dba)₃ (0.03 eq, 0.57 mmol), t-BuONa (3 eq, 57 mmol), t-Bu₃P (0.06 eq, 1.14 mmol), and 250 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 9.6 g of Compound 881 (yield: 65%, purity ≥99.9%).

### (22) Synthesis of Compound 884

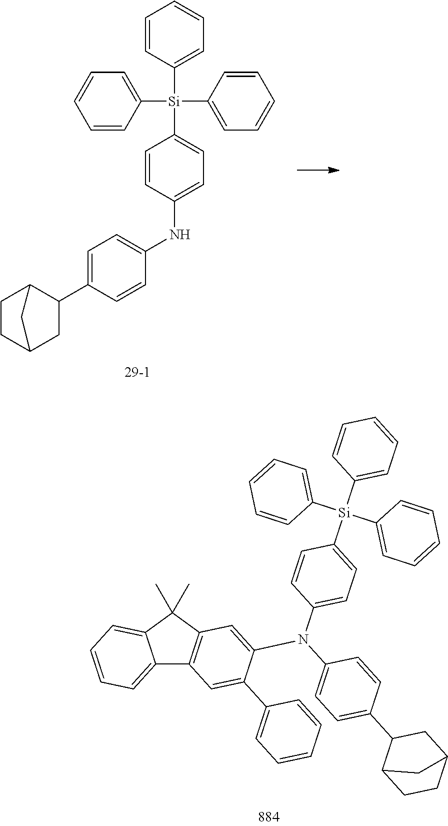 [SMILES: c1ccc([Si](c2ccccc2)(c2ccccc2)c2ccc(Nc3ccc(C4CC5CCC4C5)cc3)cc2)cc1 ; CC1(C)c2ccccc2-c2cc(-c3ccccc3)c(N(c3ccc(C4CC5CCC4C5)cc3)c3ccc([Si](c4ccccc4)(c4ccccc4)c4ccccc4)cc3)cc21]

### Synthesis of Compound 884

Intermediate 29-1 (1 eq, 10 mmol), 2-bromo-9,9-dimethyl-3-phenyl-9H-fluorene (1.1 eq, 11 mmol), Pd₂(dba)₃ (0.03 eq, 0.3 mmol), t-BuONa (3 eq, 30 mmol), t-Bu₃P (0.06 eq, 0.6 mmol), and 150 ml of toluene were put in an one-neck, round-bottom flask, followed by stirring at about 110° C. for about 2 hours. After finishing the reaction, the reaction product was worked up with H₂O and ether, and an organic layer was separated by column chromatography (eluent: methylene chloride/hexane=1/5 by volume ratio). The organic layer thus obtained was recrystallized with ether to obtain 5.3 g of Compound 884 (yield: 80%, purity ≥99.9%).

### (23) Synthesis of Compound 995

Amine Compound 995 may be synthesized by, for example, the reaction below.

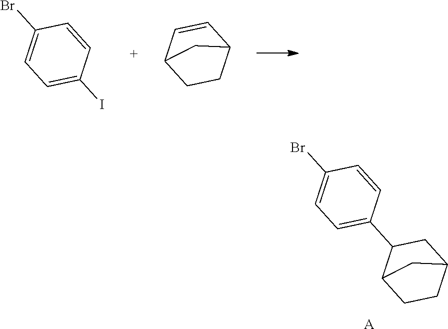

### Synthesis of Intermediate A

1-bromo-4-iodobenzene (12.9 g, 100 mmol), bicyclo[2,2,1]hept-2-ene (23.3 g), CuI (19.5 g, 100 mmol), and K₂CO₃ (25.8 g, 200 mmol) were added to 200 ml of a DMF solution, followed by stirring at about 150° C. for about 96 hours. After finishing the reaction, the temperature was reduced to room temperature, and extraction with ethyl acetate/H₂O was performed three times. After drying over anhydrous magnesium sulfate and separating by column chromatography with a mixture solvent of methylene chloride:hexane=1:10, 18.8 g (yield 80%) of Intermediate A was obtained.

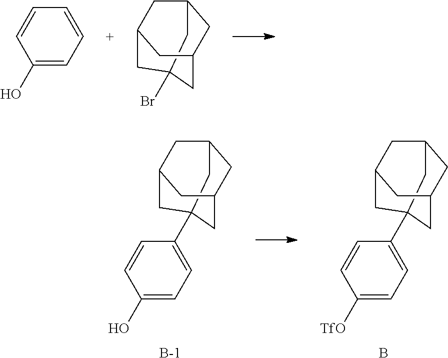

### Synthesis of Intermediate B-1

To 100 ml of phenol, 1-bromoadamantan (21.4 g, 100 mmol) was added and stirred at about 110° C. for about 24 hours. After finishing the reaction, the solid thus obtained was washed with H₂O at about 60° C. three times. The solid was dissolved in methylene chloride, dried over anhydrous magnesium sulfate to obtain Intermediate B-1 (22 g, yield 100%).

### Synthesis of Intermediate B

22 g of Intermediate B-1 and 10 g of Et₃N were dissolved in methylene chloride, and the temperature of the solution was reduced to about 0° C. 50 g of trifluoromethanesulfonic acid was added thereto for about 1 hour. Then, the temperature was raised to room temperature, and stirring was performed for about 4 hours. After finishing the reaction, the solid thus obtained was washed with Et₂O/H₂O at about 60° C. three times. The resultant product was dried over anhydrous magnesium sulfate and separated and purified by column chromatography to obtain Intermediate B (33 g, yield 90%).

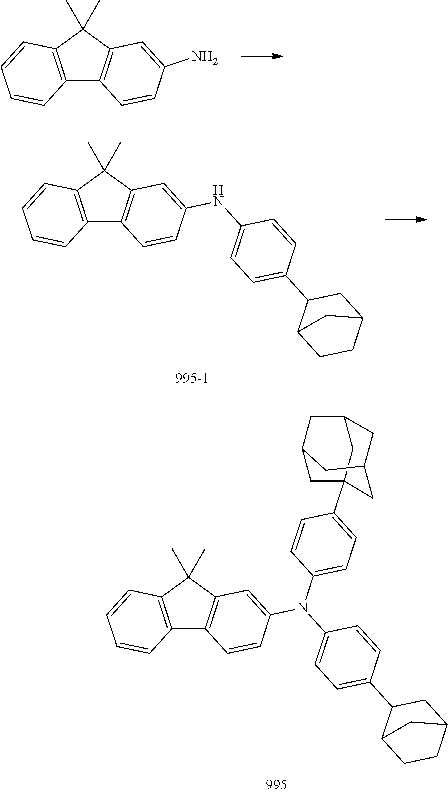

### Synthesis of Intermediate 995-1

9,9-Dimethyl-9H-fluoren-2-amine (4.2 g, 20 mmol), Intermediate A (5 g, 20 mmol), Pd₂(dba)₃ (0.915 g, 1 mol), Sphos (0.410 g, 1 m1) and NaOtBu (3.6 g, 40 mmol) were dissolved in toluene (200 m1) and stirred at about 90° C. for about 2 hours. The resultant product was extracted with Et₂O/H₂O three times. The resultant product was dried over anhydrous magnesium sulfate and separated and purified by column chromatography to obtain 6.8 g (18 mmol) of Intermediate 995-1 in a yield of 90%.

### Synthesis of Compound 995

Compound 995 (5.3 g, 9 mmol) was obtained in a yield of 90% by using substantially the same method as the synthesis of Intermediate 995-1, except for using Intermediate 995-1 instead of 9,9-dimethyl-9H-fluoren-2-amine and using Intermediate B instead of Intermediate A.

Through confirming the molecular weight and NMR results as follows, Compound 995 was identified. [C₄₄H₄₇N M+1: 590.45, ¹H NMR (500 MHz, CDCl₃) δ=7.80 (m, 2H), 7.60 (d, 1H), 7.55-7.10 (m, 12H), 2.5-0.9 (m, 32H)]

### (24) Synthesis of Compound 996

Amine Compound 996 according to an embodiment may be synthesized by, for example, the reaction below.

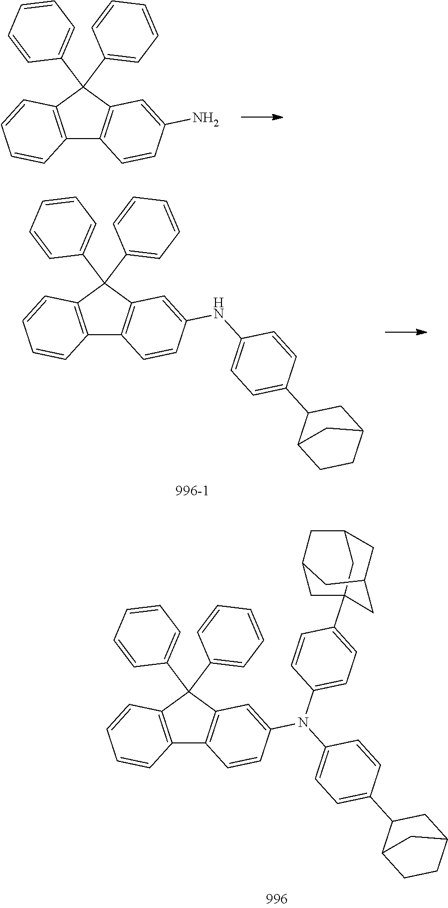

### Synthesis of Intermediate 996-1

Intermediate 996-1 (9 g, 18 mmol) was obtained in a yield of 90% by using substantially the same method as the synthesis of Intermediate 995-1, except for using 9,9-diphenyl-9H-fluoren-2-amine instead of 9,9-dimethyl-9H-fluoren-2-amine.

### Synthesis of Compound 996

Compound 996 (6.4 g, 9 mmol) was obtained in a yield of 90% by using substantially the same method as the synthesis of Compound 995, except for using Intermediate 996-1 instead of Intermediate 995-1.

Through confirming the molecular weight and NMR results as follows, Compound 996 was identified. [C₅₄H₅₁N M+1: 714.55, ¹H NMR (500 MHz, CDCl₃) δ=7.80 (m, 2H), 7.60 (d, 1H), 7.55-7.10 (m, 22H), 2.5-1.5 (m, 26H)]

### (25) Synthesis of Compound 1024

Amine Compound 1024 according to an embodiment may be synthesized by, for example, the reaction below.

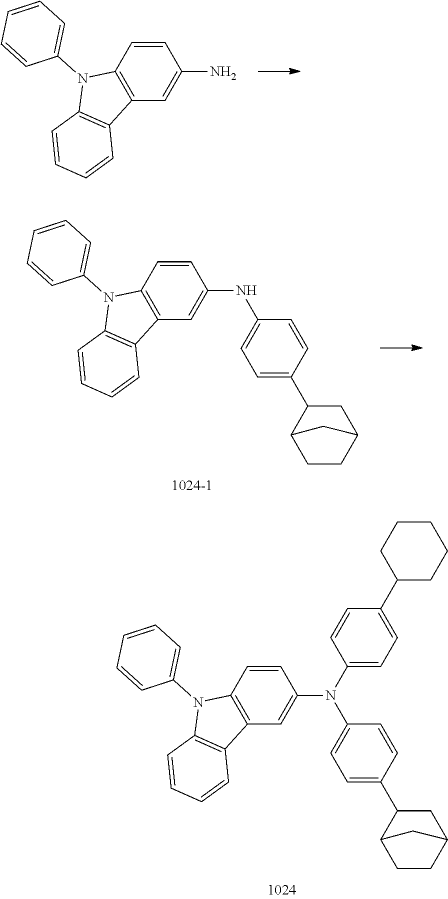 [SMILES: Nc1ccc2c(c1)c1ccccc1n2-c1ccccc1 ; c1ccc(-n2c3ccccc3c3cc(Nc4ccc(C5CC6CCC5C6)cc4)ccc32)cc1 ; c1ccc(-n2c3ccccc3c3cc(N(c4ccc(C5CCCCC5)cc4)c4ccc(C5CC6CCC5C6)cc4)ccc32)cc1]

### Synthesis of Intermediate 1024-1

Intermediate 1024-1 (7.7 g, 18 mmol) was obtained in a yield of 90% by substantially using the same method as the synthesis of Intermediate 995-1, except for using 9-phenyl-9H-carbazol-3-amine instead of 9,9-dimethyl-9H-fluoren-2-amine.

### Synthesis of Compound 1024

Compound 1024 (5.2 g, 9 mmol) was obtained in a yield of 90% by using substantially the same method as the synthesis of Compound 995, except for using Intermediate 1024-1 instead of Intermediate 995-1 and using 1-bromo-4-cyclohexylbenzene instead of Intermediate A.

Through confirming the molecular weight and NMR results as follows, Compound 1024 was identified. [C₄₃H₄₂N₂ M+1: 587.33, ¹H NMR (500 MHz, CDCl₃) δ=7.80 (m, 2H), 7.60 (d, 1H), 7.55-7.10 (m, 17H), 2.5-1.5 (m, 22H)]

### (26) Synthesis of Compound 1035

Amine Compound 1035 according to an embodiment may be synthesized by, for example, the reaction below.

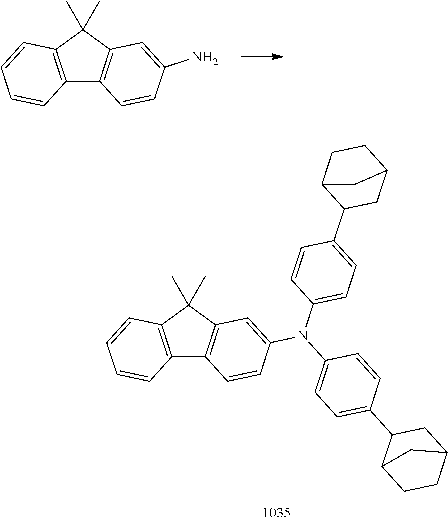 [SMILES: CC1(C)c2ccccc2-c2ccc(N)cc21 ; CC1(C)c2ccccc2-c2ccc(N(c3ccc(C4CC5CCC4C5)cc3)c3ccc(C4CC5CCC4C5)cc3)cc21]

Compound 1035 (5.2 g, 9 mmol) was obtained in a yield of 90% by using substantially the same method as the synthesis of Intermediate 995-1, except for using 9,9-dimethyl-9H-fluoren-2-amine (2.1 g, 10 mmol) instead of 9,9-dimethyl-9H-fluoren amine (4.2 g, 20 mmol).

Through confirming the molecular weight and NMR results as follows, Compound 1035 was identified. [C₄₁H₄₃N M+1: 550.52, ¹H NMR (500 MHz, CDCl₃) δ=7.80 (m, 2H), 7.60 (d, 1H), 7.55-7.10 (m, 12H), 2.5-1.5 (m, 22H), 1.3 (d, 6H)]

### (27) Synthesis of Compound 1044

Amine Compound 1044 according to an embodiment may be synthesized by, for example, the reaction below.

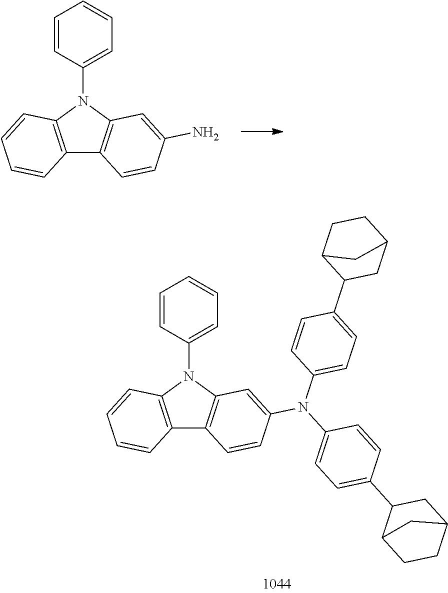 [SMILES: Nc1ccc2c3ccccc3n(-c3ccccc3)c2c1 ; c1ccc(-n2c3ccccc3c3ccc(N(c4ccc(C5CC6CCC5C6)cc4)c4ccc(C5CC6CCC5C6)cc4)cc32)cc1]

Compound 1044 (5.38 g, 9 mmol) was obtained in a yield of 90% by using substantially the same method as the synthesis of Compound 995, except for using 9-phenyl-9H-carbazol-2-amine (2.6 g, 10 mmol) instead of 9,9-dimethyl-9H-fluoren-2-amine.

Through confirming the molecular weight and NMR results as follows, Compound 1044 was identified. [C₄₄H₄₂N₂ M+1: 599.22, ¹H NMR (500 MHz, CDCl₃) δ=7.80 (m, 2H), 7.60 (d, 1H), 7.55-7.10 (m, 17H), 2.5-1.5 (m, 22H)]

### (28) Synthesis of Compound 1059

Amine Compound 1059 according to an embodiment may be synthesized by, for example, the reaction below.

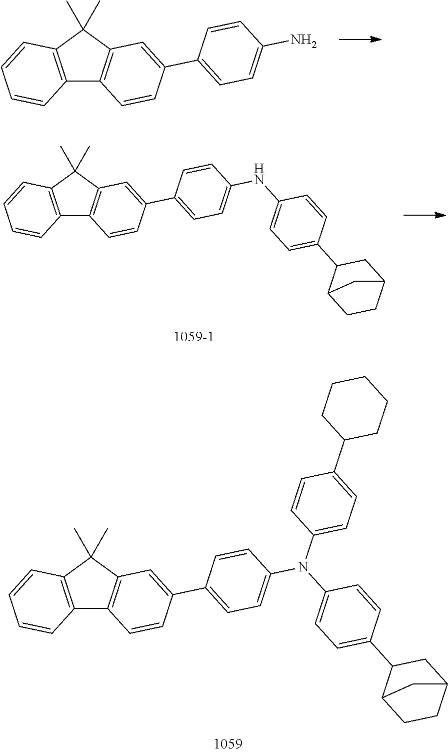

### Synthesis of Intermediate 1059-1

Intermediate 1059-1 (8.19 g, 18 mmol) was obtained in a yield of 90% by using substantially the same method as the synthesis of Intermediate 995-1, except for using 4-(9,9-dimethyl-9H-fluoren-2-yl)aniline instead of 9,9-dimethyl-9H-fluoren-2-amine.

### Synthesis of Compound 1059

Compound 1059 (5.5 g, 9 mmol) was obtained in a yield of 90% by using substantially the same method as the synthesis of Compound 995, except for using Intermediate 1059-1 instead of Intermediate 995-1 and using 1-bromo-4-cyclohexylbenzene instead of Intermediate A.

Through confirming the molecular weight and NMR results as follows, Compound 1059 was identified. [C₄₆H₄₇N M+1: 614.44, ¹H NMR (500 MHz, CDCl₃) δ=7.80 (m, 2H), 7.60 (d, 1H), 7.55-7.10 (m, 16H), 2.5-1.5 (m, 22H), 1.3 (d, 6H)]

### 2. Manufacture and Evaluation of Light Emitting Devices Including Amine Compounds

### Manufacture of Light Emitting Devices

A light emitting device of an embodiment, including an amine compound of an embodiment in a hole transport layer was manufactured by a method below. Light emitting devices of Examples 1 and 34 were manufactured using the amine compounds of Compounds 1, 6, 15, 28, 36, 43, 49, 145, 707, 803, 808, 851, 865, 868, 869, 872, 873, 876, 877, 880, 881, 884, 995, 996, 1024, 1035, 1044, and 1059, which are the aforementioned Example Compounds, as materials of a hole transport layer. Comparative Example 1 to Comparative Example 14 correspond to light emitting devices manufactured using Comparative Compound C1 to Comparative Compound C11 as materials of a hole transport layer.

### Example Compounds

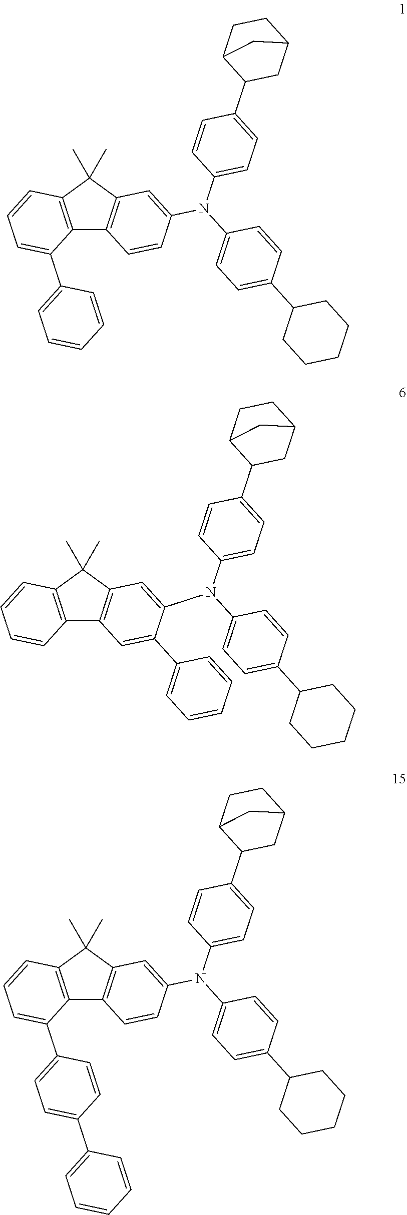 [SMILES: CC1(C)c2cc(N(c3ccc(C4CCCCC4)cc3)c3ccc(C4CC5CCC4C5)cc3)ccc2-c2c(-c3ccccc3)cccc21 ; CC1(C)c2ccccc2-c2cc(-c3ccccc3)c(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(-c4ccccc4)cc3)cccc21]

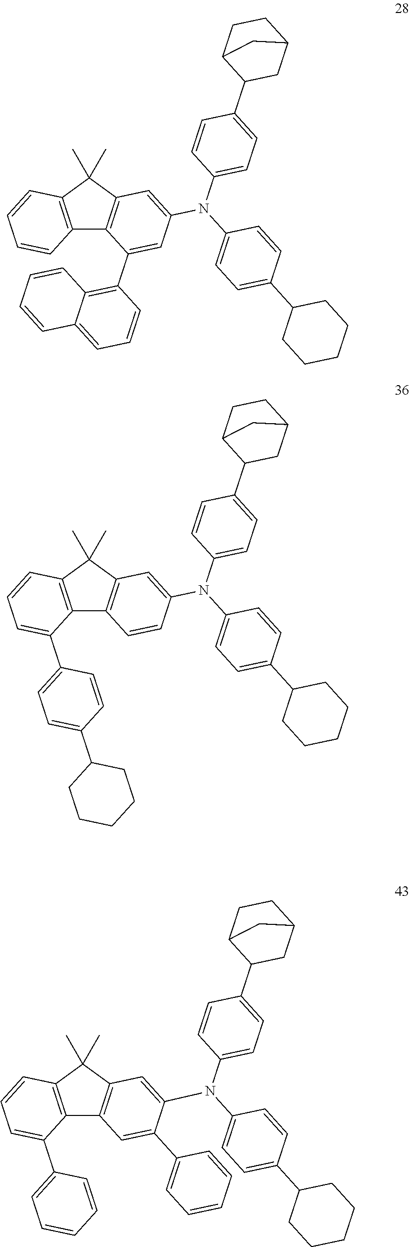 [SMILES: CC1(C)c2ccccc2-c2c(-c3cccc4ccccc34)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(C4CCCCC4)cc3)cccc21 ; CC1(C)c2cc(N(c3ccc(C4CCCCC4)cc3)c3ccc(C4CC5CCC4C5)cc3)c(-c3ccccc3)cc2-c2c(-c3ccccc3)cccc21]

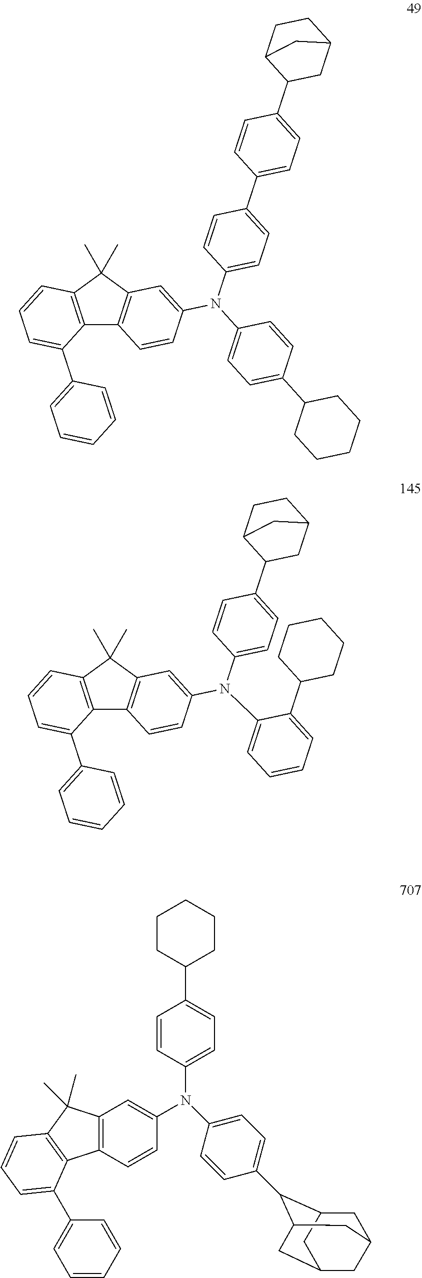 [SMILES: CC1(C)c2cc(N(c3ccc(-c4ccc(C5CC6CCC5C6)cc4)cc3)c3ccc(C4CCCCC4)cc3)ccc2-c2c(-c3ccccc3)cccc21 ; CC1(C)c2cc(N(c3ccc(C4CC5CCC4C5)cc3)c3ccccc3C3CCCCC3)ccc2-c2c(-c3ccccc3)cccc21 ; CC1(C)c2cc(N(c3ccc(C4CCCCC4)cc3)c3ccc(C4C5CC6CC(C5)CC4C6)cc3)ccc2-c2c(-c3ccccc3)cccc21]

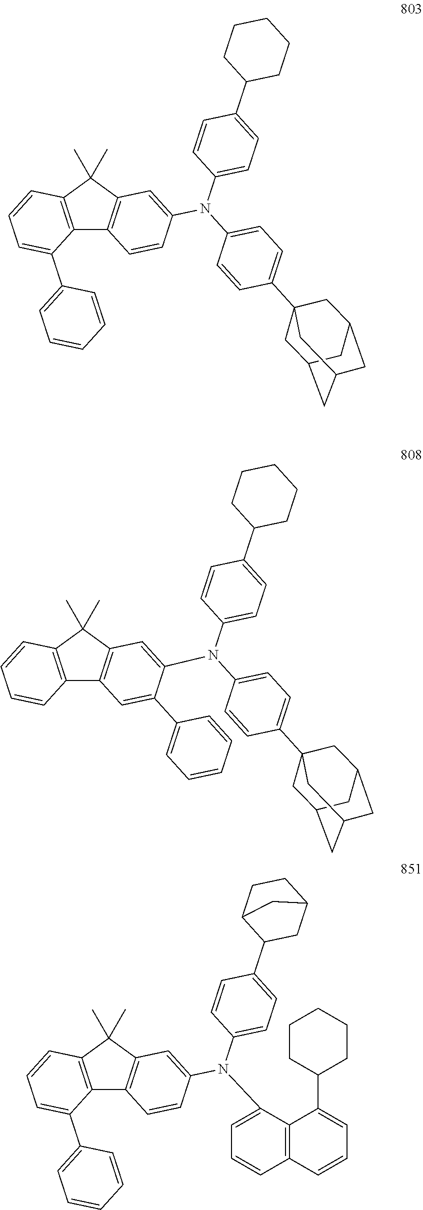 [SMILES: CC1(C)c2cc(N(c3ccc(C4CCCCC4)cc3)c3ccc(C45CC6CC(CC(C6)C4)C5)cc3)ccc2-c2c(-c3ccccc3)cccc21 ; CC1(C)c2ccccc2-c2cc(-c3ccccc3)c(N(c3ccc(C4CCCCC4)cc3)c3ccc(C45CC6CC(CC(C6)C4)C5)cc3)cc21 ; CC1(C)c2cc(N(c3ccc(C4CC5CCC4C5)cc3)c3cccc4cccc(C5CCCCC5)c34)ccc2-c2c(-c3ccccc3)cccc21]

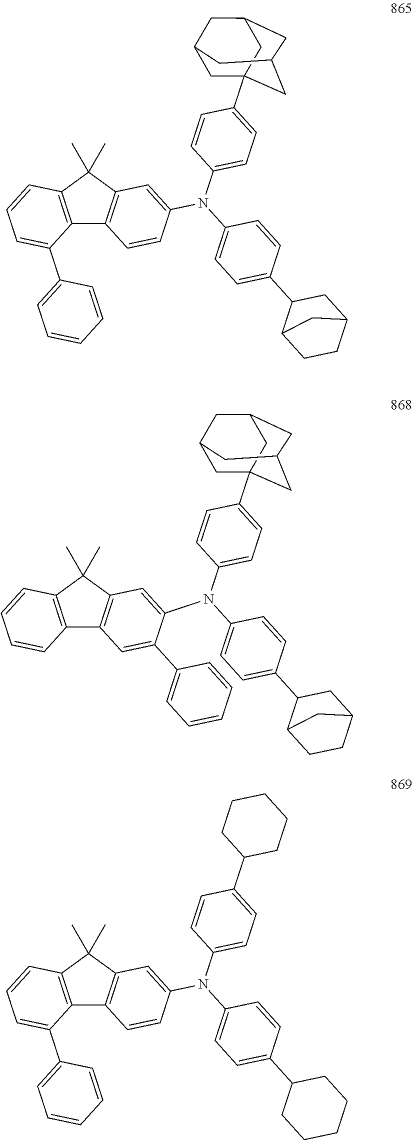 [SMILES: CC1(C)c2cc(N(c3ccc(C4CC5CCC4C5)cc3)c3ccc(C45CC6CC(CC(C6)C4)C5)cc3)ccc2-c2c(-c3ccccc3)cccc21 ; CC1(C)c2ccccc2-c2cc(-c3ccccc3)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C4CCCCC4)cc3)ccc2-c2c(-c3ccccc3)cccc21]

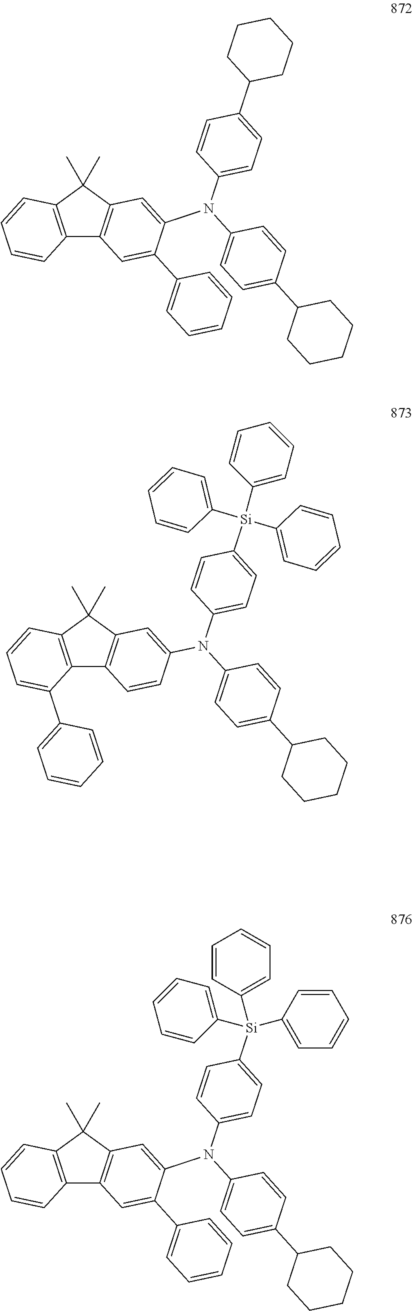 [SMILES: CC1(C)c2ccccc2-c2cc(-c3ccccc3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccccc3)cccc21 ; CC1(C)c2ccccc2-c2cc(-c3ccccc3)c(N(c3ccc(C4CCCCC4)cc3)c3ccc([Si](c4ccccc4)(c4ccccc4)c4ccccc4)cc3)cc21]

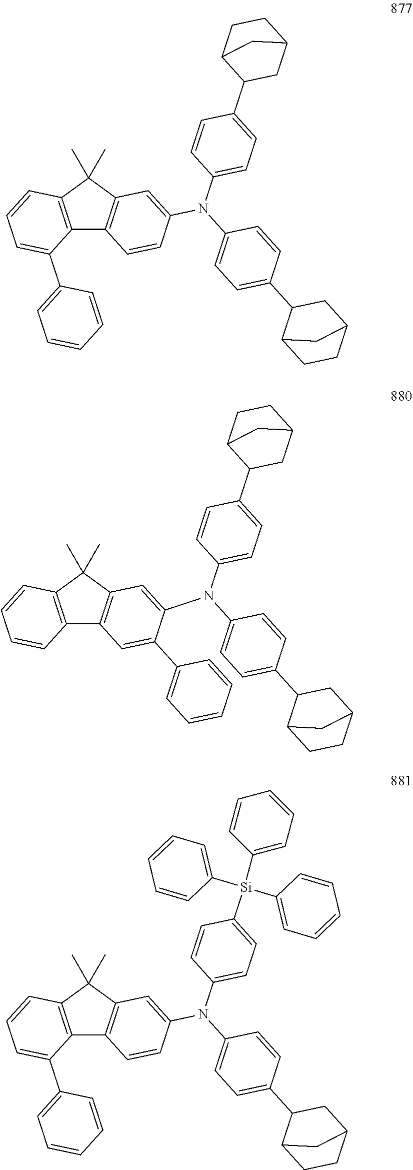 [SMILES: CC1(C)c2cc(N(c3ccc(C4CC5CCC4C5)cc3)c3ccc(C4CC5CCC4C5)cc3)ccc2-c2c(-c3ccccc3)cccc21 ; CC1(C)c2ccccc2-c2cc(-c3ccccc3)c(N(c3ccc(C4CC5CCC4C5)cc3)c3ccc(C4CC5CCC4C5)cc3)cc21 ; CC1(C)c2cc(N(c3ccc(C4CC5CCC4C5)cc3)c3ccc([Si](c4ccccc4)(c4ccccc4)c4ccccc4)cc3)ccc2-c2c(-c3ccccc3)cccc21]

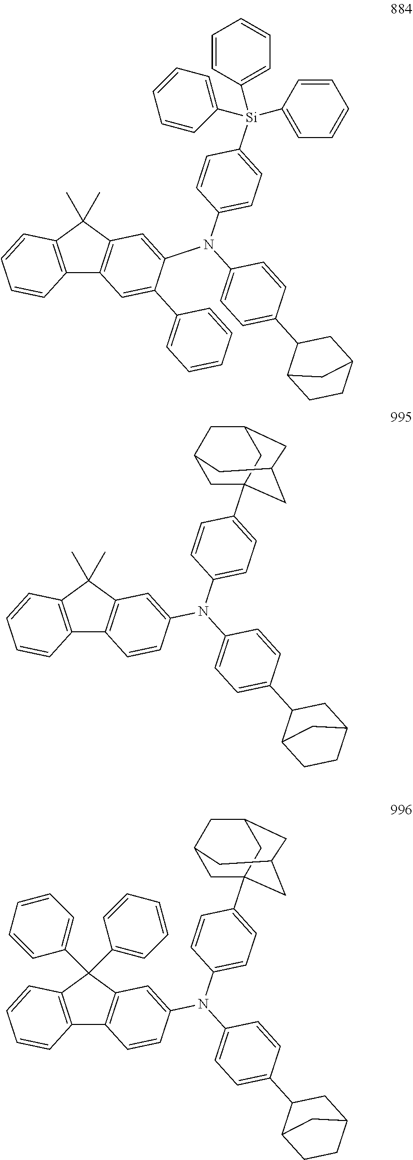 [SMILES: CC1(C)c2ccccc2-c2cc(-c3ccccc3)c(N(c3ccc(C4CC5CCC4C5)cc3)c3ccc([Si](c4ccccc4)(c4ccccc4)c4ccccc4)cc3)cc21 ; CC1(C)c2ccccc2-c2ccc(N(c3ccc(C4CC5CCC4C5)cc3)c3ccc(C45CC6CC(CC(C6)C4)C5)cc3)cc21 ; c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1]

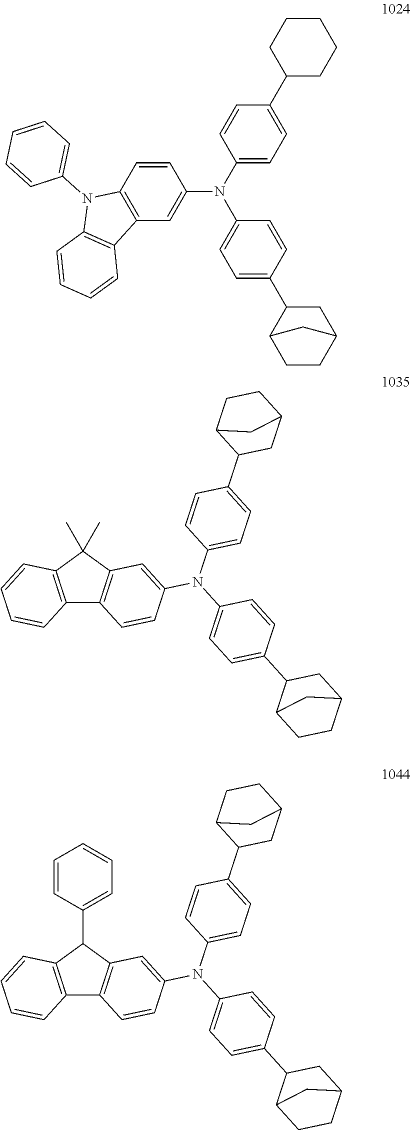

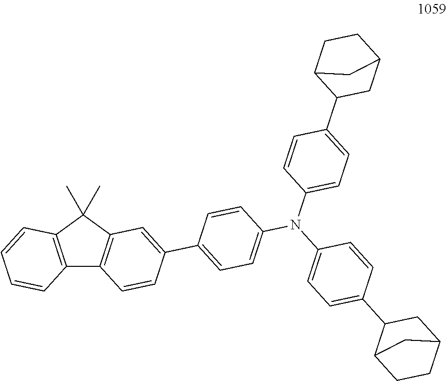 [SMILES: CC1(C)c2ccccc2-c2ccc(-c3ccc(N(c4ccc(C5CC6CCC5C6)cc4)c4ccc(C5CC6CCC5C6)cc4)cc3)cc21]

Comparative Compound C1 to Comparative Compound C11 below were used for the manufacture of the devices of Comparative Example 1 to Comparative Example 14.

### Comparative Compounds

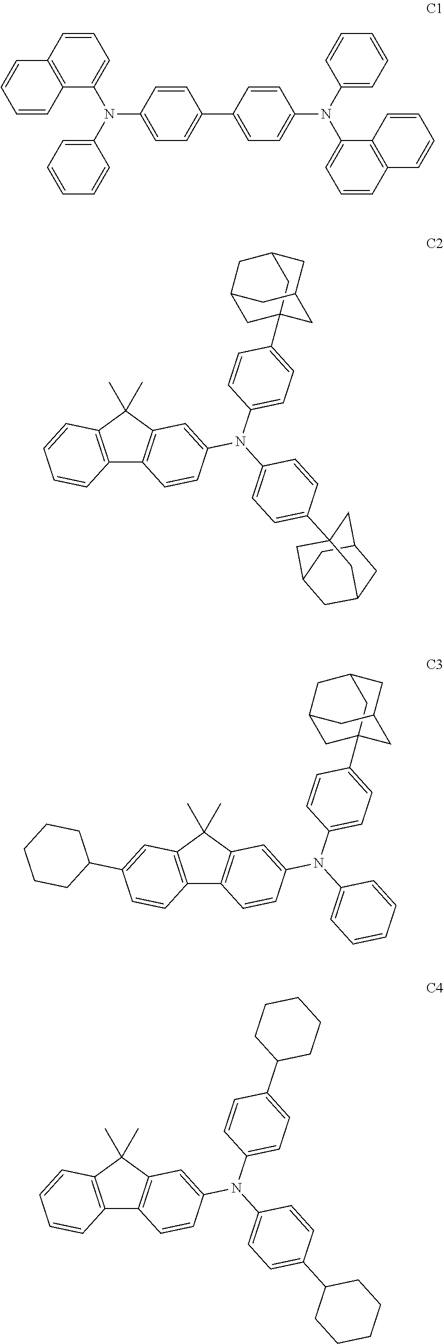 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccc(C45CC6CC(CC(C6)C4)C5)cc3)c3ccc(C45CC6CC(CC(C6)C4)C5)cc3)cc21 ; CC1(C)c2cc(C3CCCCC3)ccc2-c2ccc(N(c3ccccc3)c3ccc(C45CC6CC(CC(C6)C4)C5)cc3)cc21 ; CC1(C)c2ccccc2-c2ccc(N(c3ccc(C4CCCCC4)cc3)c3ccc(C4CCCCC4)cc3)cc21]

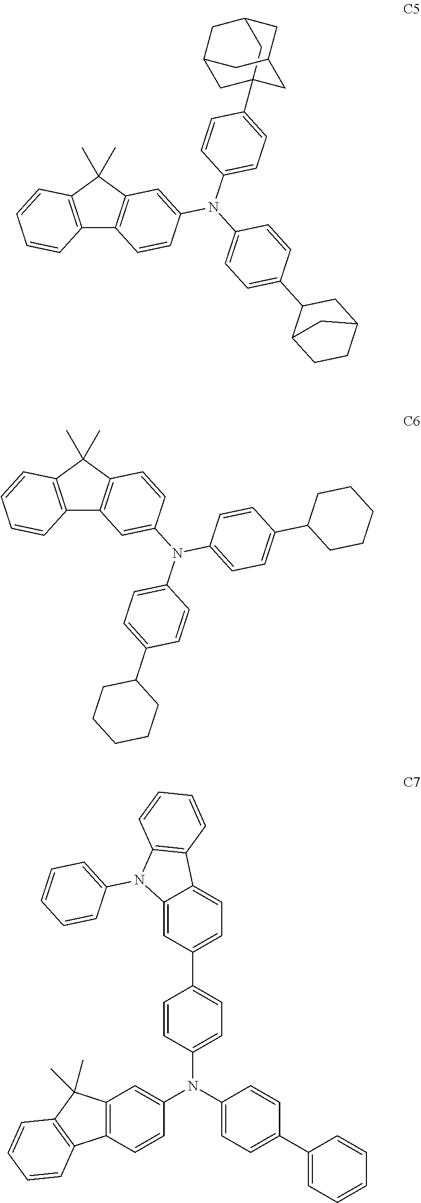 [SMILES: CC1(C)c2ccccc2-c2ccc(N(c3ccc(C4CC5CCC4C5)cc3)c3ccc(C45CC6CC(CC(C6)C4)C5)cc3)cc21 ; CC1(C)c2ccccc2-c2cc(N(c3ccc(C4CCCCC4)cc3)c3ccc(C4CCCCC4)cc3)ccc21 ; CC1(C)c2ccccc2-c2ccc(N(c3ccc(-c4ccccc4)cc3)c3ccc(-c4ccc5c6ccccc6n(-c6ccccc6)c5c4)cc3)cc21]

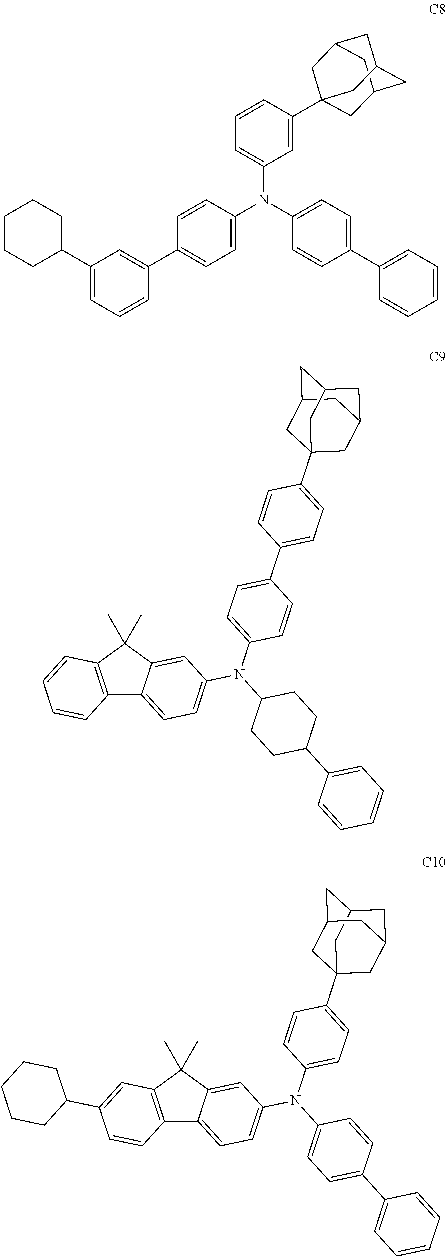 [SMILES: c1ccc(-c2ccc(N(c3ccc(-c4cccc(C5CCCCC5)c4)cc3)c3cccc(C45CC6CC(CC(C6)C4)C5)c3)cc2)cc1 ; CC1(C)c2ccccc2-c2ccc(N(c3ccc(-c4ccc(C56CC7CC(CC(C7)C5)C6)cc4)cc3)C3CCC(c4ccccc4)CC3)cc21 ; CC1(C)c2cc(C3CCCCC3)ccc2-c2ccc(N(c3ccc(-c4ccccc4)cc3)c3ccc(C45CC6CC(CC(C6)C4)C5)cc3)cc21]

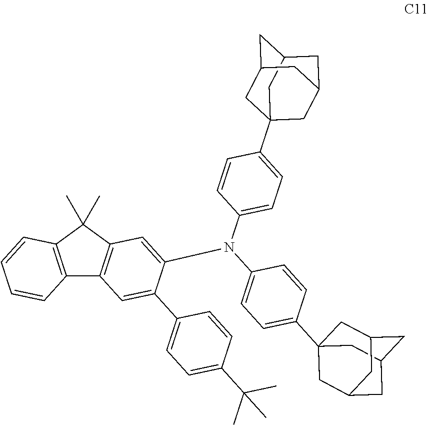 [SMILES: CC(C)(C)c1ccc(-c2cc3c(cc2N(c2ccc(C45CC6CC(CC(C6)C4)C5)cc2)c2ccc(C45CC6CC(CC(C6)C4)C5)cc2)C(C)(C)c2ccccc2-3)cc1]

### Example 1

An ITO glass substrate having a sheet resistance of 15 Ω/cm² (1200 Å) from Corning Co. was cut into a size of 50 mm×50 mm×0.7 mm, and washed with isopropyl alcohol and ultrapure water, cleaned with ultrasonic waves for about 5 minutes, exposed to UV for about 30 minutes and treated with ozone. Then, 4,4′,4″-tris{N,-(2-naphthyl)-N-phenylamino}-triphenylamine (2-TNATA) was vacuum deposited to a thickness of about 600 Å to form a hole injection layer. After that, Example Compound 1 was vacuum deposited to a thickness of about 300 Å to form a hole transport layer.

On the hole transport layer, blue fluorescence hosts of 9,10-di(naphthalen-2-yl)anthracene (DNA) and 4,4′-bis[2-(4-(N,N-diphenylamino)phenyl)vinyl]biphenyl (DPAVBi) were co-deposited in a ratio of 98:2 to form an emission layer having a thickness of about 300 Å.

On the emission layer, an electron transport layer was formed using tris(8-hydroxyquinolino)aluminum (Alq₃) to a thickness of about 300 Å, and LiF was deposited to a thickness of about 10 Å to form an electron injection layer. On the electron injection layer, a second electrode having a thickness of about 3,000 Å was formed using aluminum (Al).

The compounds used for the manufacture of the light emitting devices of the Examples and Comparative Examples are shown below.

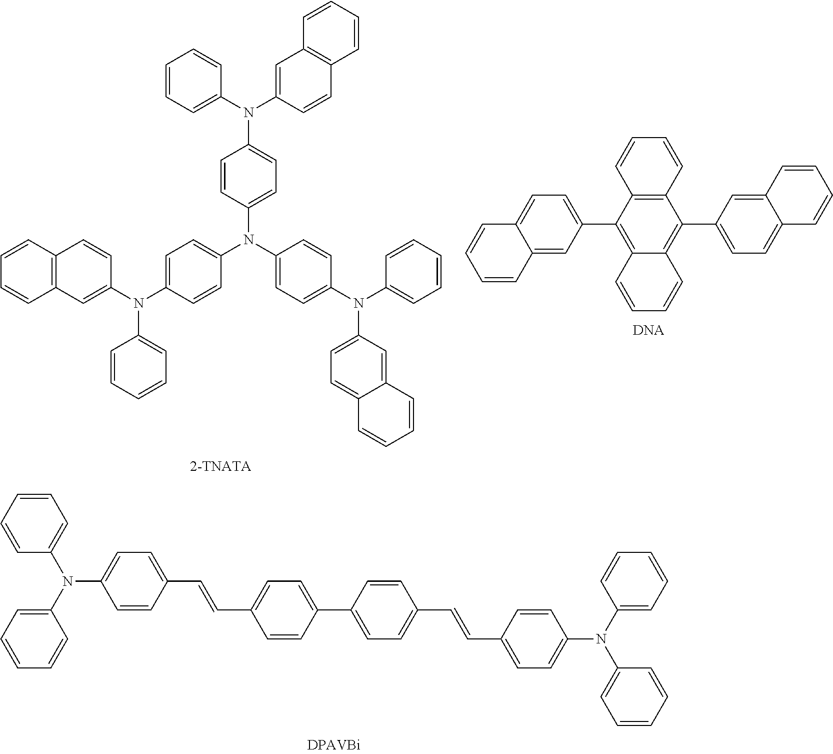

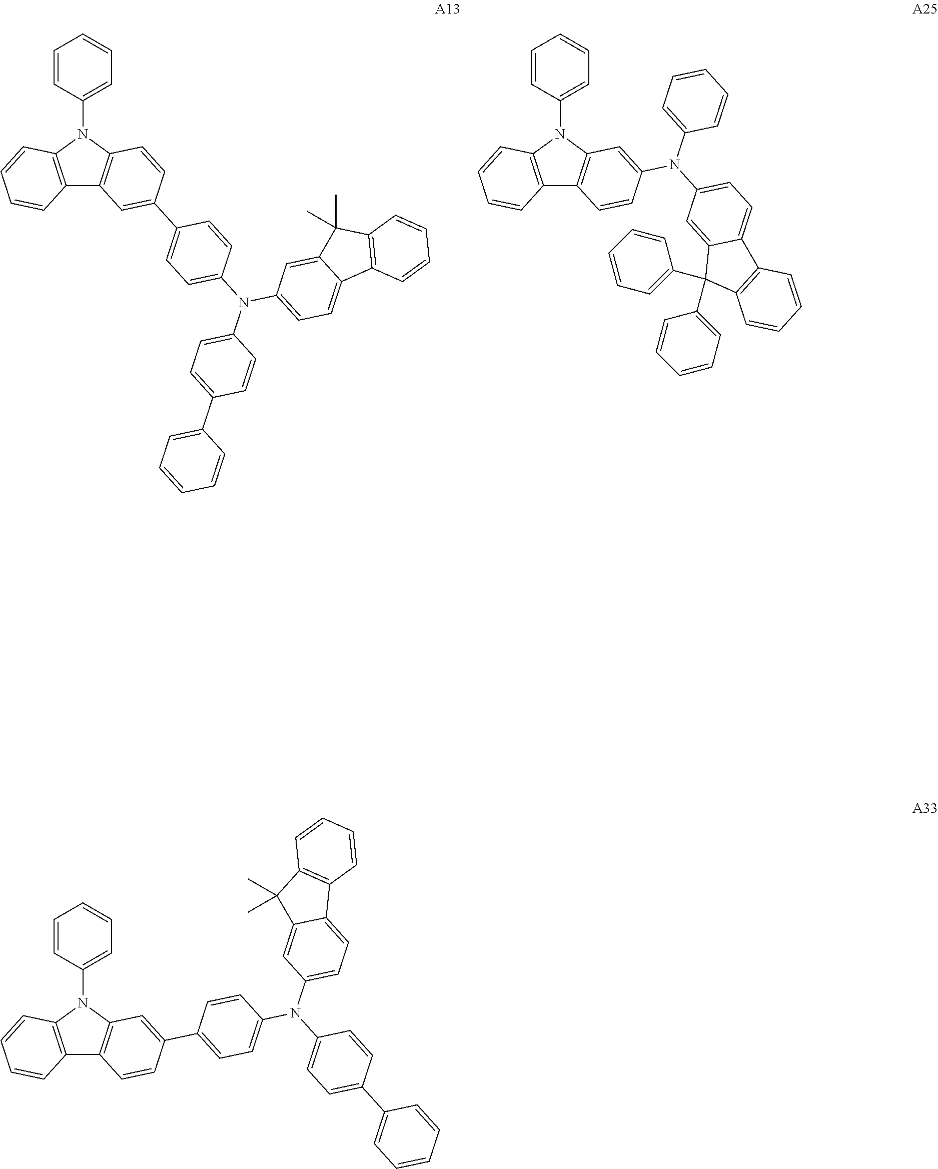 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2ccc(N(c3ccc(-c4ccccc4)cc3)c3ccc(-c4ccc5c6ccccc6n(-c6ccccc6)c5c4)cc3)cc21]

### Example 2

A light emitting device was manufactured by substantially the same method as Example 1, except for using Example Compound 6 instead of Example Compound 1 for forming the hole transport layer, when compared to Example 1.

### Example 3

A light emitting device was manufactured by substantially the same method as Example 1, except for using Example Compound 15 instead of Example Compound 1 for forming the hole transport layer, when compared to Example 1.

### Example 4

A light emitting device was manufactured by substantially the same method as Example 1, except for using Example Compound 28 instead of Example Compound 1 for forming the hole transport layer, when compared to Example 1.

### Example 5

A light emitting device was manufactured by substantially the same method as Example 1, except for using Example Compound 36 instead of Example Compound 1 for forming the hole transport layer, when compared to Example 1.

### Example 6

A light emitting device was manufactured by substantially the same method as Example 1, except for using Example Compound 43 instead of Example Compound 1 for forming the hole transport layer, when compared to Example 1.

### Example 7

A light emitting device was manufactured by substantially the same method as Example 1, except for using Example Compound 49 instead of Example Compound 1 for forming the hole transport layer, when compared to Example 1.

### Example 8

A light emitting device was manufactured by substantially the same method as Example 1, except for using Example Compound 145 instead of Example Compound 1 for forming the hole transport layer, when compared to Example 1.

### Example 9

A light emitting device was manufactured by substantially the same method as Example 1, except for using Example Compound 707 instead of Example Compound 1 for forming the hole transport layer, when compared to Example 1.

### Example 10

A light emitting device was manufactured by substantially the same method as Example 1, except for using Example Compound 851 instead of Example Compound 1 for forming the hole transport layer, when compared to Example 1.

### Example 11

A light emitting device was manufactured by substantially the same method as Example 1, except for using Example Compound 865 instead of Example Compound 1 for forming the hole transport layer, when compared to Example 1.

### Example 12

A light emitting device was manufactured by substantially the same method as Example 1, except for using Example Compound 868 instead of Example Compound 1 for forming the hole transport layer, when compared to Example 1.

### Example 13

A light emitting device was manufactured by substantially the same method as Example 1, except for using Example Compound 803 instead of Example Compound 1 for forming the hole transport layer, when compared to Example 1.

### Example 14

A light emitting device was manufactured by substantially the same method as Example 1, except for using Example Compound 808 instead of Example Compound 1 for forming the hole transport layer, when compared to Example 1.

### Example 15

A light emitting device was manufactured by substantially the same method as Example 1, except for using Example Compound 869 instead of Example Compound 1 for forming the hole transport layer, when compared to Example 1.

### Example 16

A light emitting device was manufactured by substantially the same method as Example 1, except for using Example Compound 876 instead of Example Compound 1 for forming the hole transport layer, when compared to Example 1.

### Example 17

A light emitting device was manufactured by substantially the same method as Example 1, except for using Example Compound 995 instead of Example Compound 1 for forming the hole transport layer, when compared to Example 1.

### Example 18

A light emitting device was manufactured by substantially the same method as Example 1, except for using Example Compound 996 instead of Example Compound 1 for forming the hole transport layer, when compared to Example 1.

### Example 19

A light emitting device was manufactured by substantially the same method as Example 1, except for using Example Compound 1024 instead of Example Compound 1 for forming the hole transport layer, when compared to Example 1.

### Example 20

A light emitting device was manufactured by substantially the same method as Example 1, except for using Example Compound 1035 instead of Example Compound 1 for forming the hole transport layer, when compared to Example 1.

### Example 21

A light emitting device was manufactured by substantially the same method as Example 1, except for using Example Compound 1044 instead of Example Compound 1 for forming the hole transport layer, when compared to Example 1.

### Example 22

A light emitting device was manufactured by substantially the same method as Example 1, except for using Example Compound 1059 instead of Example Compound 1 for forming the hole transport layer, when compared to Example 1.

### Example 23

An ITO glass substrate having a sheet resistance of 15 Ω/cm² (1200 Å) from Corning Co. was cut into a size of 50 mm×50 mm×0.7 mm, and washed with isopropyl alcohol and ultrapure water, cleaned with ultrasonic waves for about 5 minutes, exposed to UV for about 30 minutes and treated with ozone. Then, 4,4′,4″-tris{N,-(2-naphthyl)-N-phenylamino}-triphenylamine (2-TNATA) was vacuum deposited to a thickness of about 600 Å to form a hole injection layer.

On the hole injection layer, Example Compound 1 was vacuum deposited to a thickness of about 100-400 Å to form a first hole transport layer, Example Compound A13 was vacuum deposited on the first hole transport layer to a thickness of about 100-400 Å to form a second hole transport layer, and Example Compound 1 was vacuum deposited on the second hole transport layer to a thickness of about 100-400 Å to form a third hole transport layer.

On the hole transport layer, blue fluorescence hosts of 9,10-di(naphthalen-2-yl)anthracene (DNA) and 4,4′-bis[2-(4-(N,N-diphenylamino)phenyl)vinyl]biphenyl (DPAVBi) were co-deposited in a ratio of 98:2 to form an emission layer having a thickness of about 300 Å.

On the emission layer, an electron transport layer was formed using tris(8-hydroxyquinolino)aluminum (Alq₃) to a thickness of about 300 Å, and LiF was deposited to a thickness of about 10 Å to form an electron injection layer. On the electron injection layer, a second electrode having a thickness of about 3,000 Å was formed using aluminum (Al).

The compounds used for the manufacture of the light emitting devices of the Examples and Comparative Examples are shown below.

### Example 24

A light emitting device was manufactured by substantially the same method as Example 23, except for using Example Compound 6 instead of Example Compound 1 for forming the first hole transport layer and the third hole transport layer, when compared to Example 23.

### Example 25

A light emitting device was manufactured by substantially the same method as Example 23, except for using Example Compound 873 instead of Example Compound 1 for forming the first hole transport layer and the third hole transport layer, when compared to Example 23.

### Example 26

A light emitting device was manufactured by substantially the same method as Example 23, except for using Example Compound 876 instead of Example Compound 1 for forming the first hole transport layer and the third hole transport layer, when compared to Example 23.

### Example 27

A light emitting device was manufactured by substantially the same method as Example 23, except for using Example Compound 877 instead of Example Compound 1 for forming the first hole transport layer and the third hole transport layer, and using Example Compound A25 instead of Example Compound A13 for forming the second hole transport layer, when compared to Example 23.

### Example 28

A light emitting device was manufactured by substantially the same method as Example 23, except for using Example Compound 880 instead of Example Compound 1 for forming the first hole transport layer and the third hole transport layer, and using Example Compound A25 instead of Example Compound A13 for forming the second hole transport layer, when compared to Example 23.

### Example 29

A light emitting device was manufactured by substantially the same method as Example 23, except for using Example Compound 881 instead of Example Compound 1 for forming the first hole transport layer and the third hole transport layer, and using Example Compound A25 instead of Example Compound A13 for forming the second hole transport layer, when compared to Example 23.

### Example 30

A light emitting device was manufactured by substantially the same method as Example 23, except for using Example Compound 884 instead of Example Compound 1 for forming the first hole transport layer and the third hole transport layer, and using Example Compound A25 instead of Example Compound A13 for forming the second hole transport layer, when compared to Example 23.

### Example 31

A light emitting device was manufactured by substantially the same method as Example 23, except for using Example Compound A33 instead of Example Compound A13 for forming the second hole transport layer, when compared to Example 23.

### Example 32

A light emitting device was manufactured by substantially the same method as Example 23, except for using Example Compound 6 instead of Example Compound 1 for forming the first hole transport layer and the third hole transport layer, and using Example Compound A33 instead of Example Compound A13 for forming the second hole transport layer, when compared to Example 23.

### Example 33

A light emitting device was manufactured by substantially the same method as Example 23, except for using Example Compound 995 instead of Example Compound 1 for forming the first hole transport layer and the third hole transport layer, when compared to Example 23.

### Example 34

A light emitting device was manufactured by substantially the same method as Example 23, except for using Example Compound 995 instead of Example Compound 1 for forming the first hole transport layer and the third hole transport layer, and using Example Compound A25 instead of Example Compound A13 for forming the second hole transport layer, when compared to Example 23.

### Comparative Example 1

A light emitting device was manufactured by substantially the same method as Example 1, except for using Comparative Compound C1 instead of Example Compound 1 for forming the hole transport layer, when compared to Example 1.

### Comparative Example 2

A light emitting device was manufactured by substantially the same method as Example 1, except for using Comparative Compound C2 instead of Example Compound 1 for forming the hole transport layer, when compared to Example 1.

### Comparative Example 3

A light emitting device was manufactured by substantially the same method as Example 1, except for using Comparative Compound C4 instead of Example Compound 1 for forming the hole transport layer, when compared to Example 1.

### Comparative Example 4

A light emitting device was manufactured by substantially the same method as Example 1, except for using Comparative Compound C5 instead of Example Compound 1 for forming the hole transport layer, when compared to Example 1.

### Comparative Example 5

A light emitting device was manufactured by substantially the same method as Example 1, except for using Comparative Compound C7 instead of Example Compound 1 for forming the hole transport layer, when compared to Example 1.

### Comparative Example 6

A light emitting device was manufactured by substantially the same method as Example 1, except for using Comparative Compound C8 instead of Example Compound 1 for forming the hole transport layer, when compared to Example 1.

### Comparative Example 7

A light emitting device was manufactured by substantially the same method as Example 1, except for using Comparative Compound C9 instead of Example Compound 1 for forming the hole transport layer, when compared to Example 1.

### Comparative Example 8

A light emitting device was manufactured by substantially the same method as Example 1, except for using Comparative Compound C10 instead of Example Compound 1 for forming the hole transport layer, when compared to Example 1.

### Comparative Example 9

A light emitting device was manufactured by substantially the same method as Example 1, except for using Comparative Compound C11 instead of Example Compound 1 for forming the hole transport layer, when compared to Example 1.

### Comparative Example 10

A light emitting device was manufactured by substantially the same method as Example 23, except for using Comparative Compound C3 instead of Example Compound 1 for forming the first hole transport layer and the third hole transport layer, when compared to Example 23.

### Comparative Example 11

A light emitting device was manufactured by substantially the same method as Example 23, except for using Comparative Compound C4 instead of Example Compound 1 for forming the first hole transport layer and the third hole transport layer, when compared to Example 23.

### Comparative Example 12

A light emitting device was manufactured by substantially the same method as Example 23, except for using Comparative Compound C5 instead of Example Compound 1 for forming the first hole transport layer and the third hole transport layer, when compared to Example 23.

### Comparative Example 13

A light emitting device was manufactured by substantially the same method as Example 23, except for using Comparative Compound C6 instead of Example Compound 1 for forming the first hole transport layer and the third hole transport layer, when compared to Example 23.

### Comparative Example 14

A light emitting device was manufactured by substantially the same method as Example 23, except for using Comparative Compound C4 instead of Example Compound 1 for forming the first hole transport layer and the third hole transport layer, and using Example Compound 217 instead of Example Compound 197 for forming the second hole transport layer, when compared to Example 23.

### Evaluation of Properties of Light Emitting Device

In Table 1, the evaluation results of the light emitting devices of Example 1 to Example 22, and Comparative Example 1 to Comparative Example 9 are shown. In Table 2, the evaluation results of the light emitting devices of Example 23 to Example 34, and Comparative Example 10 to Comparative Example 14 are shown. In Table 1 and Table 2, the luminance, emission efficiency and half-life of the light emitting devices manufactured are shown.

In the evaluation results on the properties of the Examples and Comparative Examples, shown in Table 1 and Table 2, the voltage and current density were measured using V7000 OLED IVL Test System, (Polaronix). The emission efficiency shows efficiency values measured at a current density of about 50 mA/cm²⁻. The half-life shows half-life values measured at a current density of about 100 mA/cm²⁻.

TABLE 1

| | Hole | | | | | | Half life |
|---|---|---|---|---|---|---|---|
| Device | transport | Driving | Current | | | | (hr |
| manufacturing | layer | voltage | density | Luminance | Efficiency | Luminous | @100 |
| example | material | (V) | (mA/cm²) | (cd/m²) | (cd/A) | color | mA/cm²) |
| Example 1 | Compound | 4.75 | 50 | 3500 | 7.0 | Blue | 550 |
| | 1 | | | | | | |
| Example 2 | Compound | 4.61 | 50 | 3450 | 6.9 | Blue | 545 |
| | 6 | | | | | | |
| Example 3 | Compound | 4.61 | 50 | 3440 | 6.88 | Blue | 575 |
| | 15 | | | | | | |
| Example 4 | Compound | 4.50 | 50 | 3400 | 6.8 | Blue | 530 |
| | 28 | | | | | | |
| Example 5 | Compound | 4.79 | 50 | 3500 | 7 | Blue | 540 |
| | 36 | | | | | | |
| Example 6 | Compound | 4.88 | 50 | 3405 | 6.81 | Blue | 520 |
| | 43 | | | | | | |
| Example 7 | Compound | 4.91 | 50 | 3440 | 6.88 | Blue | 545 |
| | 49 | | | | | | |
| Example 8 | Compound | 4.38 | 50 | 3485 | 6.97 | Blue | 535 |
| | 145 | | | | | | |
| Example 9 | Compound | 4.60 | 50 | 3485 | 6.97 | Blue | 545 |
| | 707 | | | | | | |
| Example 10 | Compound | 4.44 | 50 | 3420 | 6.84 | Blue | 535 |
| | 851 | | | | | | |
| Example 11 | Compound | 4.32 | 50 | 3775 | 7.55 | Blue | 520 |
| | 865 | | | | | | |
| Example 12 | Compound | 4.83 | 50 | 3560 | 7.12 | Blue | 510 |
| | 868 | | | | | | |
| Example 13 | Compound | 4.85 | 50 | 3610 | 7.22 | Blue | 510 |
| | 803 | | | | | | |
| Example 14 | Compound | 4.81 | 50 | 3765 | 7.53 | Blue | 555 |
| | 808 | | | | | | |
| Example 15 | Compound | 4.80 | 50 | 3545 | 7.09 | Blue | 540 |
| | 869 | | | | | | |
| Example 16 | Compound | 4.80 | 50 | 3335 | 6.67 | Blue | 560 |
| | 876 | | | | | | |
| Example 17 | Compound | 4.75 | 50 | 3075 | 6.15 | Blue | 520 |
| | 995 | | | | | | |
| Example 18 | Compound | 4.72 | 50 | 3060 | 6.12 | Blue | 510 |
| | 996 | | | | | | |
| Example 19 | Compound | 4.75 | 50 | 3010 | 6.02 | Blue | 560 |
| | 1024 | | | | | | |
| Example 20 | Compound | 4.73 | 50 | 2965 | 5.93 | Blue | 615 |
| | 1035 | | | | | | |
| Example 21 | Compound | 4.72 | 50 | 3045 | 6.09 | Blue | 570 |
| | 1044 | | | | | | |
| Example 22 | Compound | 4.72 | 50 | 3115 | 6.23 | Blue | 500 |
| | 1059 | | | | | | |
| Comparative | Comparative | 7.01 | 50 | 2645 | 5.29 | Blue | 258 |
| Example 1 | Compound | | | | | | |
| | C1 | | | | | | |
| Comparative | Comparative | 6.59 | 50 | 2805 | 5.61 | Blue | 335 |
| Example 2 | Compound | | | | | | |
| | C2 | | | | | | |
| Comparative | Comparative | 6.42 | 50 | 2770 | 5.54 | Blue | 305 |
| Example 3 | Compound | | | | | | |
| | C4 | | | | | | |
| Comparative | Comparative | 6.78 | 50 | 2825 | 5.65 | Blue | 340 |
| Example 4 | Compound | | | | | | |
| | C5 | | | | | | |
| Comparative | Comparative | 6.7 | 50 | 2800 | 5.6 | Blue | 300 |
| Example 5 | Compound | | | | | | |
| | C7 | | | | | | |
| Comparative | Comparative | 6.8 | 50 | 2845 | 5.69 | Blue | 360 |
| Example 6 | Compound | | | | | | |
| | C8 | | | | | | |
| Comparative | Comparative | 6.5 | 50 | 2760 | 5.52 | Blue | 355 |
| Example 7 | Compound | | | | | | |
| | C9 | | | | | | |
| Comparative | Comparative | 6.3 | 50 | 3005 | 6.01 | Blue | 380 |
| Example 8 | Compound | | | | | | |
| | C10 | | | | | | |
| Comparative | Comparative | 7.3 | 50 | 2895 | 5.79 | Blue | 370 |
| Example 9 | Compound | | | | | | |
| | C11 | | | | | | |

Referring to the results of Table 1, it can be seen that the Examples of the light emitting devices using the amine compound according to an embodiment of the present disclosure as the material of a hole transport layer emitted the same blue light, showed very low driving voltage values, and showed relatively higher luminance, emission efficiency and device life when compared to the Comparative Examples. The Example Compounds include a first fused ring combined with the nitrogen atom of an amine group and have a structure essentially including a first substituent and a second substituent, which are substituents selected from an adamantyl group, a cyclohexyl group, a bicycloheptanyl group, a bicyclooctanyl group, and a triphenylsilyl group. The first substituent and the second substituent are connected with the nitrogen atom of the amine group via linkers, and the linker may be a substituted or unsubstituted arylene group, or a substituted or unsubstituted heteroarylene group, expanding the conjugation structure of the amine group. In addition, the Example Compounds may have a structure including at least one or more third substituents which are aryl groups or heteroaryl groups as substituents in addition to the amine group connected with the first fused ring, or may have a structure in which at least one selected from among the first substituent and the second substituent is a bicycloheptanyl group. The amine compound of an embodiment, having such a structure may show high thermal properties and improved hole transport capacity, and if applied to a light emitting device, the improvement of the emission efficiency and the increase of the life of the light emitting device may be accomplished. In some embodiments, the light emitting device of an embodiment includes the amine compound of an embodiment as the material of a hole transport layer of the light emitting device, and the efficiency and life of the light emitting device may be improved.

In the case of Comparative Compound C1 included in Comparative Example 1, a first fused ring substituted at an amine group was not included, and at least one substituent selected from among an adamantyl group, a cyclohexyl group, a bicycloheptanyl group, a bicyclooctanyl group, and a triphenylsilyl group was not included, and it can be seen that if applied to a light emitting device, luminance and emission efficiency were reduced, and half-life was reduced when compared to the Example Compounds.

The case of Comparative Compound C2 included in Comparative Example 2, had a structure in which an amine group was combined with a fluorenyl group, and an adamantyl group is included as a substituent, but did not include a substituent additionally connected with the fluorenyl group other than the amine group. Accordingly, it can be seen that hole transport capacity and molecular stability were deteriorated, and if applied to a light emitting device, luminance and emission efficiency were deteriorated, and half-life was reduced when compared to the Example Compounds.

When comparing Examples 2 and 5, with Comparative Examples 3 and 4, Comparative Compound C4 and Comparative Compound C5, included in Comparative Examples 3 and 4 had a structure in which a fluorenyl group was combined with an amine group, and had a structure including a cyclohexyl group, an adamantyl group, or a bicycloheptanyl group as a substituent, but it can be seen that emission efficiency was reduced, and half-life was reduced. It is thought that the results were shown because, Comparative Compound C4 and Comparative Compound C5 did not include a substituent additionally connected with the fluorenyl group in addition to the amine group, and showed degraded molecular stability when compared to the Example Compounds, and if applied to a light emitting device, heat resistance and charge transfer properties were degraded when compared to the Examples.

Comparative Compound C7 included in Comparative Example 5 included a structure in which an amine group was connected with a fluorene core, but did not include a first substituent and a second substituent as substituents connected with an amine group, as shown in the present disclosure, and it can be seen that both emission efficiency and device life were reduced when compared to the Examples.

Comparative Compound C8 included in Comparative Example 6 was a compound not including a fluorene core, and had insufficient molecular stability when compared to the Example Compounds, and it can be seen that both emission efficiency and device life were degraded when compared to the Examples.

Comparative Compound C9 included in Comparative Example 7 was a compound in which a cyclohexyl group was directly combined with an amine group, and accordingly, molecular stability was insufficient, and both emission efficiency and device life were degraded when compared to the Examples.

Comparative Compound C10 included in Comparative Example 8 included a structure in which an amine group was connected with a fluorene core but included one adamantyl group as a substituent connected with the amine group, and it can be seen that both emission efficiency and device life were degraded when compared to the Examples. The amine compounds of the Examples of the present disclosure included both a first substituent and a second substituent connected with an amine group and may show improved emission efficiency and life characteristics. In addition, the case of Comparative Compound 8 had a structure in which a cyclohexyl group was connected with a fluorene core, but with such a structure, the expansion of π-conjugation was difficult, molecular stability was deteriorated during driving, and emission efficiency and device life characteristics were deteriorated.

Comparative Compound C11 included in Comparative Example 9 included a structure in which an amine group was connected with a fluorene core, but included two adamantyl groups as substituents connected with an amine group, and it can be seen that both emission efficiency and device life were reduced when compared to the Examples.

TABLE 2

| | | Second | | | | | | | |
|---|---|---|---|---|---|---|---|---|---|
| Device | First hole | hole | Third hole | Driving | Current | | | | Half |
| manufacturing | transport | transport | transport | voltage | density | Luminance | Efficiency | Luminous | life |
| example | layer | layer | layer | (V) | (mA/cm²) | (cd/m²) | (cd/A) | color | (hr) |
| Example 23 | Compound 1 | Compound | Compound 1 | 4.56 | 50 | 3300 | 6.60 | Blue | 615 |
| | | A13 | | | | | | | |
| Example 24 | Compound 6 | Compound | Compound 6 | 4.63 | 50 | 3300 | 6.85 | Blue | 630 |
| | | A13 | | | | | | | |
| Example 25 | Compound | Compound | Compound | 4.69 | 50 | 3300 | 7.00 | Blue | 620 |
| | 873 | A13 | 873 | | | | | | |
| Example 26 | Compound | Compound | Compound | 4.69 | 50 | 3300 | 6.95 | Blue | 640 |
| | 876 | A13 | 876 | | | | | | |
| Example 27 | Compound | Compound | Compound | 4.59 | 50 | 3270 | 6.54 | Blue | 610 |
| | 877 | A25 | 877 | | | | | | |
| Example 28 | Compound | Compound | Compound | 4.62 | 50 | 3270 | 6.75 | Blue | 625 |
| | 880 | A25 | 880 | | | | | | |
| Example 29 | Compound | Compound | Compound | 4.66 | 50 | 3270 | 6.50 | Blue | 635 |
| | 881 | A25 | 881 | | | | | | |
| Example 30 | Compound | Compound | Compound | 4.66 | 50 | 3270 | 6.84 | Blue | 630 |
| | 884 | A25 | 884 | | | | | | |
| Example 31 | Compound 1 | Compound | Compound 1 | 4.3 | 50 | 3495 | 6.99 | Blue | 685 |
| | | A33 | | | | | | | |
| Example 32 | Compound 6 | Compound | Compound 6 | 4.5 | 50 | 3490 | 6.98 | Blue | 675 |
| | | A33 | | | | | | | |
| Example 33 | Compound | Compound | Compound | 4.72 | 50 | 3300 | 6.60 | Blue | 580 |
| | 995 | A13 | 995 | | | | | | |
| Example 34 | Compound | Compound | Compound | 4.72 | 50 | 3270 | 6.54 | Blue | 520 |
| | 995 | A25 | 995 | | | | | | |
| Comparative | Comparative | Compound | Comparative | 5.66 | 50 | 3002 | 6.00 | Blue | 450 |
| Example 10 | Compound | A13 | Compound | | | | | | |
| | C3 | | C3 | | | | | | |
| Comparative | Comparative | Compound | Comparative | 5.30 | 50 | 2880 | 5.76 | Blue | 385 |
| Example 11 | Compound | A13 | Compound | | | | | | |
| | C4 | | C4 | | | | | | |
| Comparative | Comparative | Compound | Comparative | 5.49 | 50 | 2925 | 5.85 | Blue | 390 |
| Example 12 | Compound | A13 | Compound | | | | | | |
| | C5 | | C5 | | | | | | |
| Comparative | Comparative | Compound | Comparative | 5.60 | 50 | 3000 | 6.00 | Blue | 450 |
| Example 13 | Compound | A13 | Compound | | | | | | |
| | C6 | | C5 | | | | | | |
| Comparative | Comparative | Compound | Comparative | 5.6 | 50 | 3000 | 6.00 | Blue | 305 |
| Example 14 | Compound | A33 | Compound | | | | | | |
| | C4 | | C4 | | | | | | |

Referring to the results of Table 1 and Table 2, it can be seen that the Examples including multiple hole transport layers showed improved device characteristics of emission efficiency or device life when compared to the Comparative Examples. In addition, with respect to the emission efficiency properties of the light emitting devices, it can be seen that Examples 23 to 34, including multiple hole transport layers showed improved emission efficiency and device life when compared to the Examples of Examples 1 to 22, including one hole transport layer.

The light emitting device of an embodiment may show improved device properties of high efficiency and long-life characteristics.

The amine compound of an embodiment may be included in the hole transport region of a light emitting device to contribute to the increase of the efficiency and life of the light emitting device.

Although the embodiments of the present disclosure have been described, it should be understood that the present disclosure should not be limited to these embodiments but various changes and modifications can be made by one ordinary skilled in the art within the spirit and scope of the appended claims, and equivalents thereof.

What is claimed is:

1. A light emitting device, comprising:
a first electrode;
a second electrode on the first electrode; and
at least one functional layer between the first electrode and the second electrode, and comprising an amine compound represented by the following Formula 1: 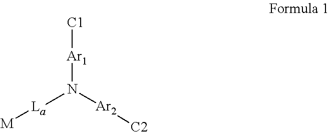
in Formula 1,
Ar₁ and Ar₂ are each independently a substituted or unsubstituted arylene group of 6 to 30 ring-forming carbon atoms, or a substituted or unsubstituted heteroarylene group of 2 to 30 ring-forming carbon atoms,
Lₐ is a direct linkage, a substituted or unsubstituted arylene group of 6 to 30 ring-forming carbon atoms, or a substituted or unsubstituted heteroarylene group of 2 to 30 ring-forming carbon atoms,
C1 and C2 are each independently a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group,
a case where both C1 and C2 are substituted or unsubstituted adamantyl groups is excluded, and
M is represented by any one selected from among the following Formula 1-a to Formula 1-c: 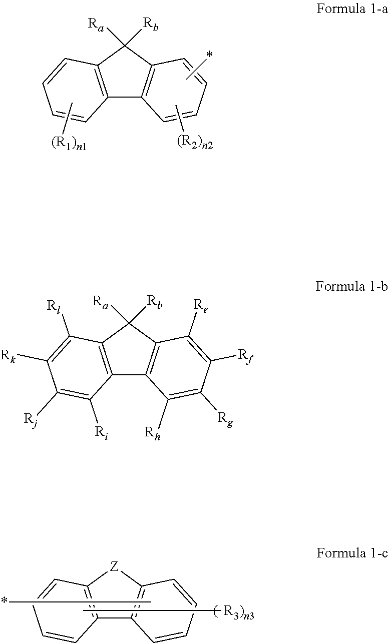
in Formula 1-a to Formula 1-c,
Z is NR₄, O, or S,
R₁ and R₂ are each independently a substituted or unsubstituted aryl group of 6 to 30 ring-forming carbon atoms, or a substituted or unsubstituted heteroaryl group of 2 to 30 ring-forming carbon atoms, and
Rₐ to Rₗ, R₃, and R₄ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
in Formula 1-b, any one selected from among R_{c} to Rₗ is a part connected with Formula 1,
in Formula 1-a and Formula 1-c, 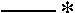 [SMILES: *C]
is a part connected with Formula 1,
in a case where M in Formula 1 is represented by Formula 1-b or Formula 1-c, at least one selected from among C1 and C2 in Formula 1 is a bicycloheptanyl group,
n1 is an integer of 0 to 4,
n2 is an integer of 0 to 3,
where the sum of n1 and n2 is 1 or more, and
n3 is an integer of 0 to 7.

2. The light emitting device of claim 1, wherein the at least one functional layer comprises an emission layer, a hole transport region between the first electrode and the emission layer, and an electron transport region between the emission layer and second electrode, and
the hole transport region comprises the amine compound.

3. The light emitting device of claim 1, wherein the amine compound represented by Formula 1 is a monoamine compound.

4. The light emitting device of claim 1, wherein the amine compound represented by Formula 1 is represented by any one selected from among the following Formula 2-1 to Formula 2-8: 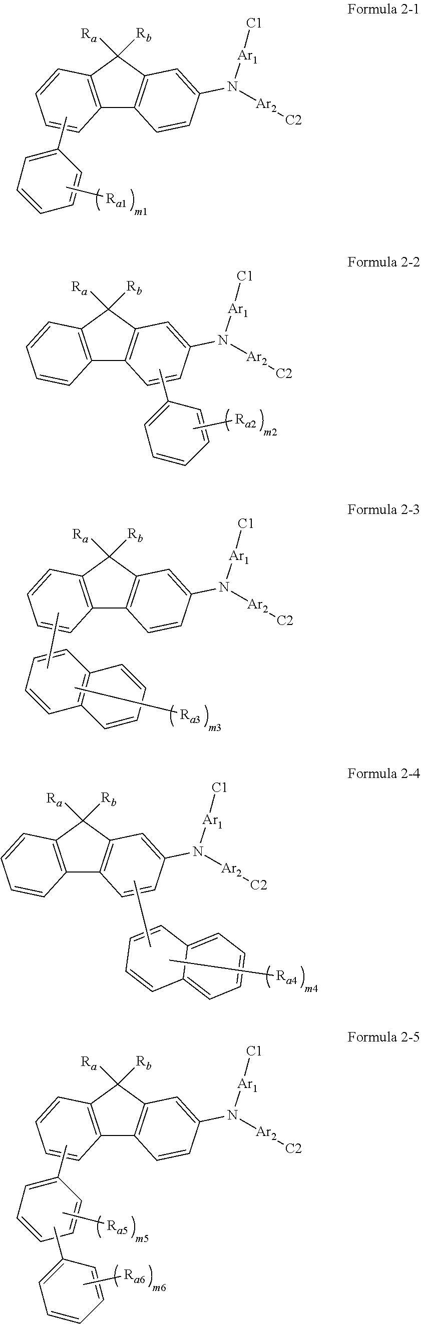 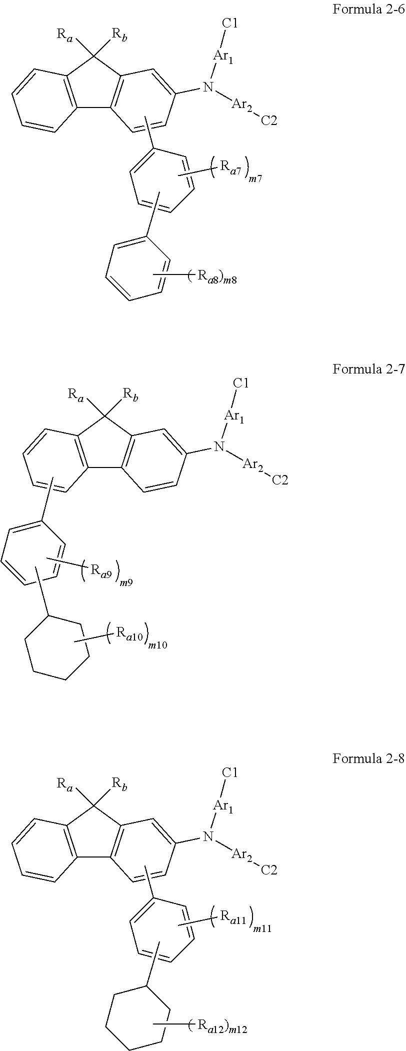
in Formula 2-1 to Formula 2-8,
Rₐ₁ to Rₐ₁₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m1, m2, m6, and m8 are each independently an integer of 0 to 5,
m3 and m4 are each independently an integer of 0 to 7,
m5, m7, m9, and m11 are each independently an integer of 0 to 4,
m10, and m12 are each independently an integer of 0 to 11, and
Rₐ, R_{b}, Ar₁, Ar₂, C1 and C2 are the same as defined with respect to Formula 1 and Formula 1-a.

5. The light emitting device of claim 1, wherein the amine compound represented by Formula 1 is represented by the following Formula 3-1 or Formula 3-2: 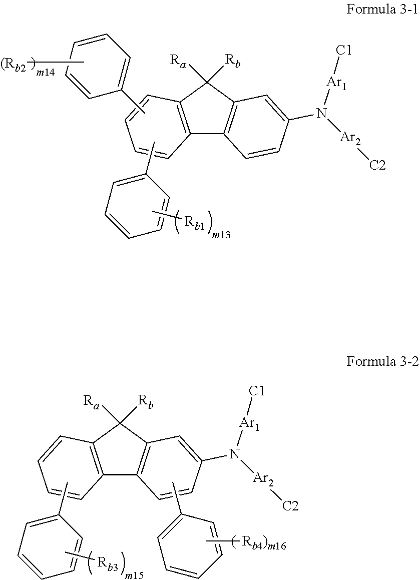
in Formula 3-1 and Formula 3-2,
R_{b1} to R_{b4} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m11 to m14 are each independently an integer of 0 to 5, and
Rₐ, R_{b}, Ar₁, Ar₂, C1 and C2 are the same as defined with respect to Formula 1 and Formula 1-a.

6. The light emitting device of claim 1, wherein the amine compound represented by Formula 1 is represented by any one selected from among the following Formula 4-1 to Formula 4-15: 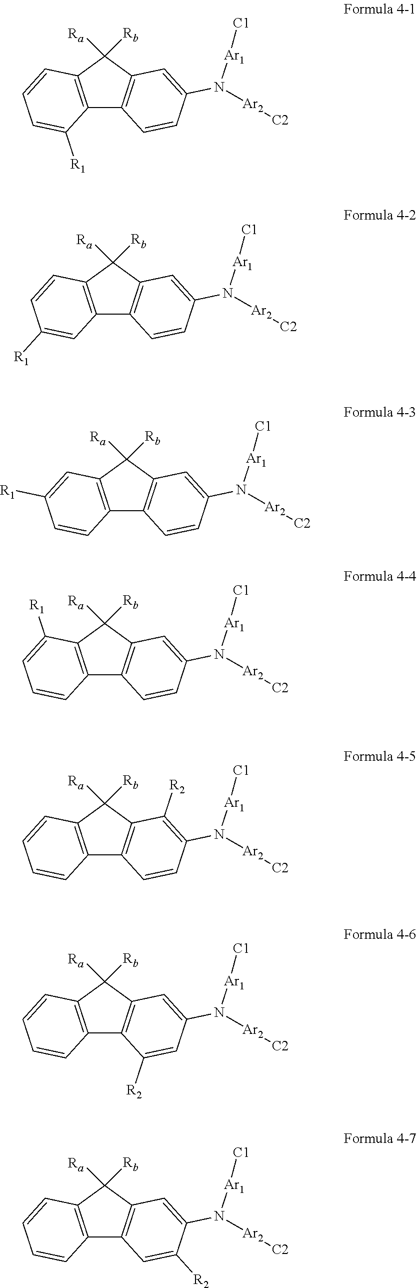 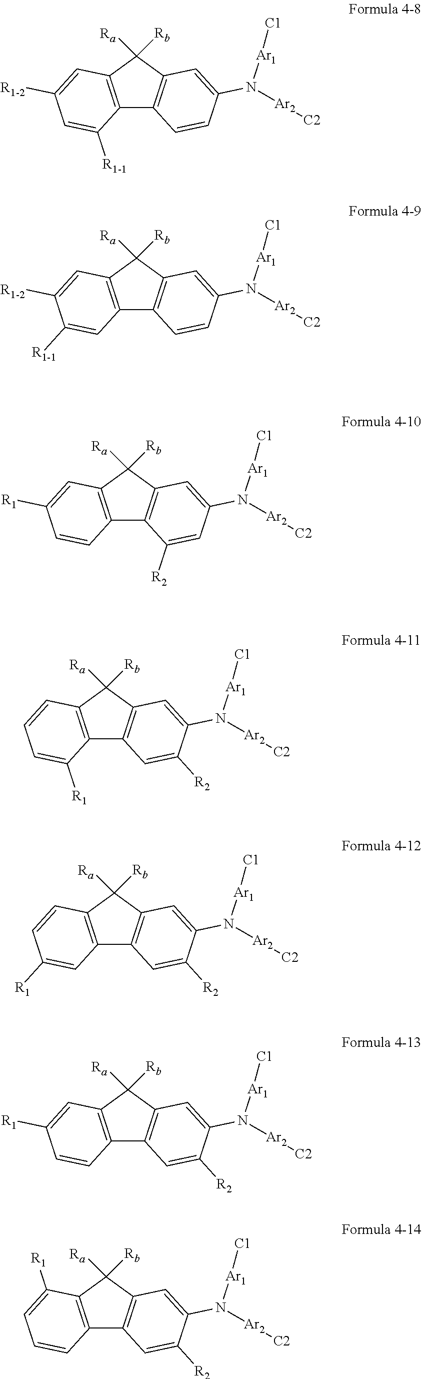 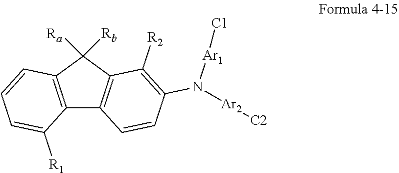
in Formula 4-1 to Formula 4-15,
R₁₋₁ and R₁₋₂ are each independently a substituted or unsubstituted aryl group of 6 to 30 ring-forming carbon atoms, or a substituted or unsubstituted heteroaryl group of 2 to 30 ring-forming carbon atoms, and
R₁, R₂, Rₐ, R_{b}, Ar₁, Ar₂, C1, and C2 are the same as defined with respect to Formula 1 and Formula 1-a.

7. The light emitting device of claim 1, wherein the amine compound represented by Formula 1 is represented by any one selected from among the following Formula 5-1 to Formula 5-3: 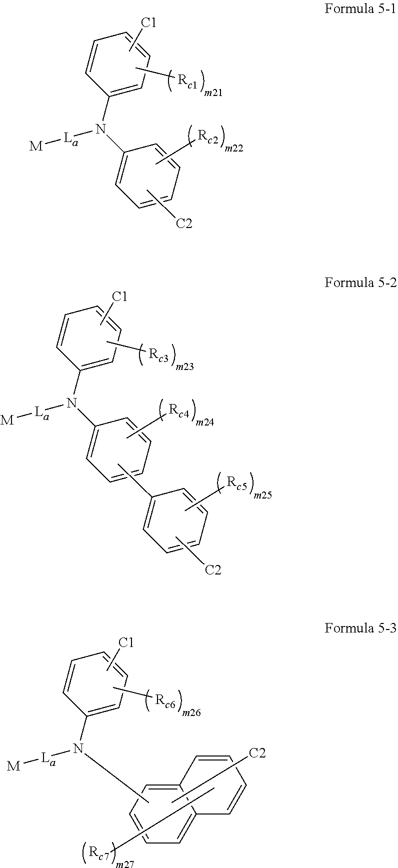
in Formula 5-1 to Formula 5-3,
R_{c1} to R_{c7} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m21 to m26 are each independently an integer of 0 to 4,
m27 is an integer of 0 to 6, and
M, Lₐ, C1, and C2 are the same as defined with respect to Formula 1.

8. The light emitting device of claim 1, wherein the amine compound represented by Formula 1 is represented by any one selected from among the following Formula 6-1 to Formula 6-8: 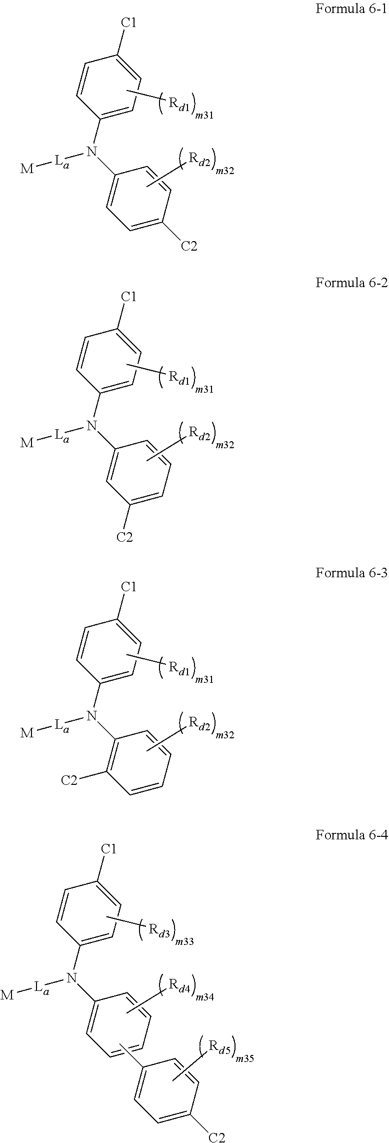 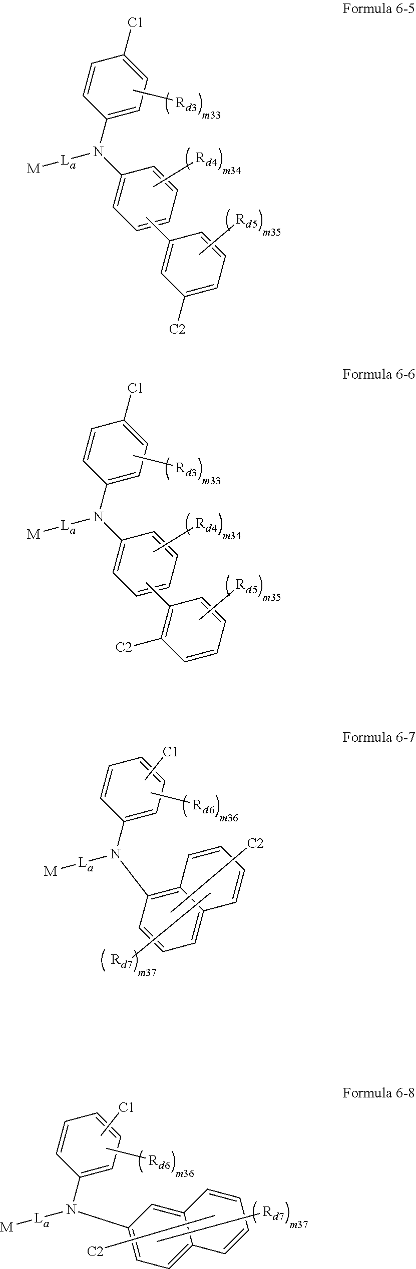
in Formula 6-1 to Formula 6-8,
R_{d1} to R_{d7} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m31 to m36 are each independently an integer of 0 to 4,
m37 is an integer of 0 to 6, and
M, Lₐ, C1, and C2 are the same as defined with respect to Formula 1.

9. The light emitting device of claim 1, wherein C1 and C2 are each independently represented by any one selected from among the following Formula 7-1 to Formula 7-8: 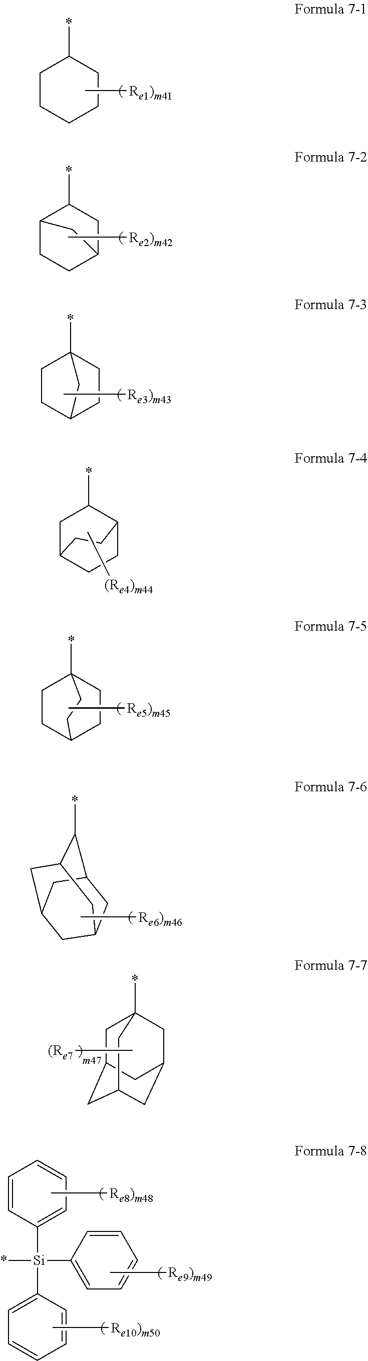
in Formula 7-1 to Formula 7-8,
Rₑ₁ to Rₑ₁₀ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m41 to m43 are each independently an integer of 0 to 11,
m44 and m45 are each independently an integer of 0 to 13,
m46 and m47 are each independently an integer of 0 to 15, and
m48 to m50 are each independently an integer of 0 to 5.

10. The light emitting device of claim 1, wherein the amine compound represented by Formula 1 is represented by any one selected from among the following Formula 8-1 to Formula 8-5: 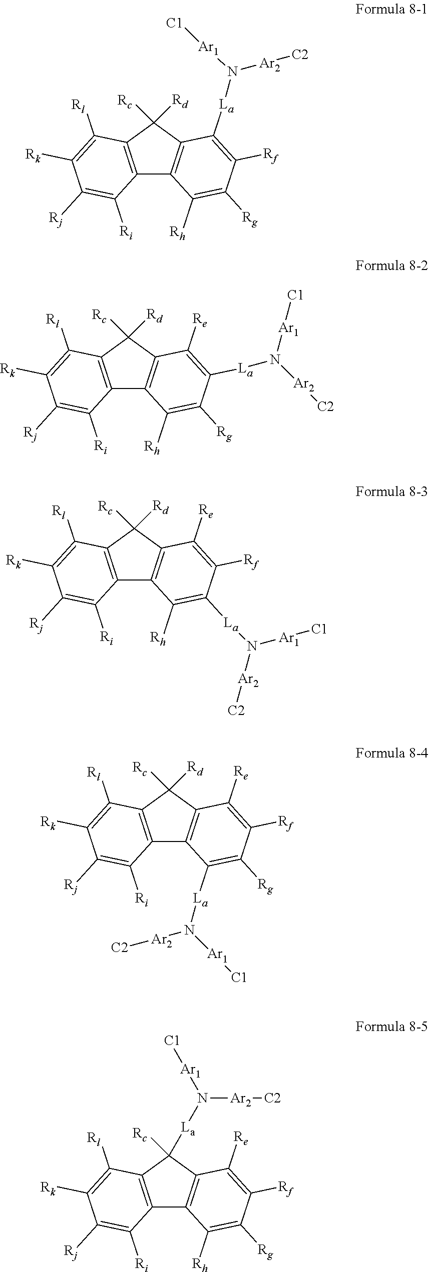
in Formula 8-1 to Formula 8-5,
at least one selected from among C1 and C2 is a substituted or unsubstituted bicycloheptanyl group, and
Ar₁, Ar₂, Lₐ, C1, C2, and R_{c} to Rₗ are the same as defined with respect to Formula 1 and Formula 1-b.

11. The light emitting device of claim 1, wherein the amine compound represented by Formula 1 is represented by any one selected from among the following Formula 9-1 to Formula 9-4: 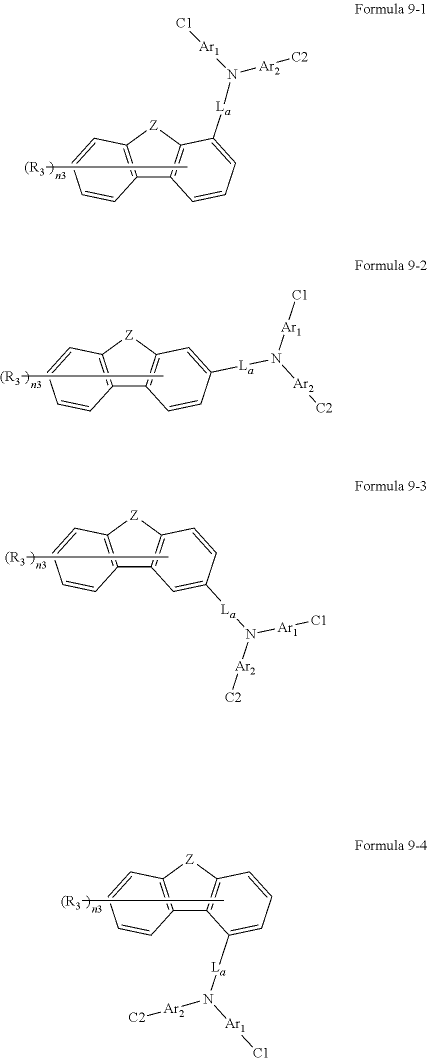
in Formula 9-1 to Formula 9-4,
at least one selected from among C1 and C2 is a substituted or unsubstituted bicycloheptanyl group, and
Z, R₃, Ar₁, Ar₂, Lₐ, n3, C1, and C2 are the same as defined with respect to Formula 1 and Formula 1-c.

12. The light emitting device of claim 1, wherein the amine compound is at least one selected from compounds in the following Compound Group 1:

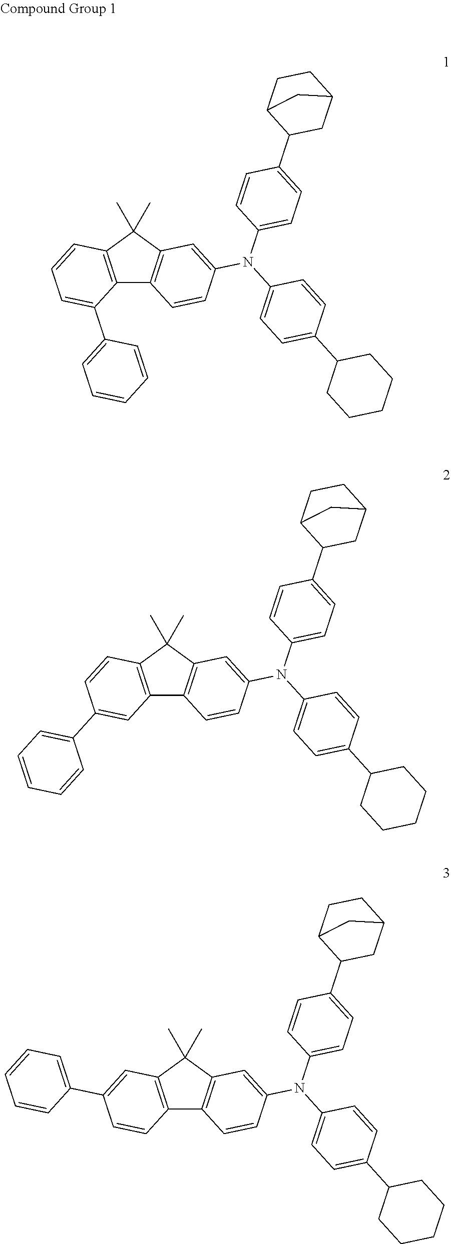 [SMILES: CC1(C)c2cc(N(c3ccc(C4CCCCC4)cc3)c3ccc(C4CC5CCC4C5)cc3)ccc2-c2c(-c3ccccc3)cccc21 ; CC1(C)c2ccc(-c3ccccc3)cc2-c2ccc(N(c3ccc(C4CCCCC4)cc3)c3ccc(C4CC5CCC4C5)cc3)cc21 ; CC1(C)c2cc(-c3ccccc3)ccc2-c2ccc(N(c3ccc(C4CCCCC4)cc3)c3ccc(C4CC5CCC4C5)cc3)cc21]

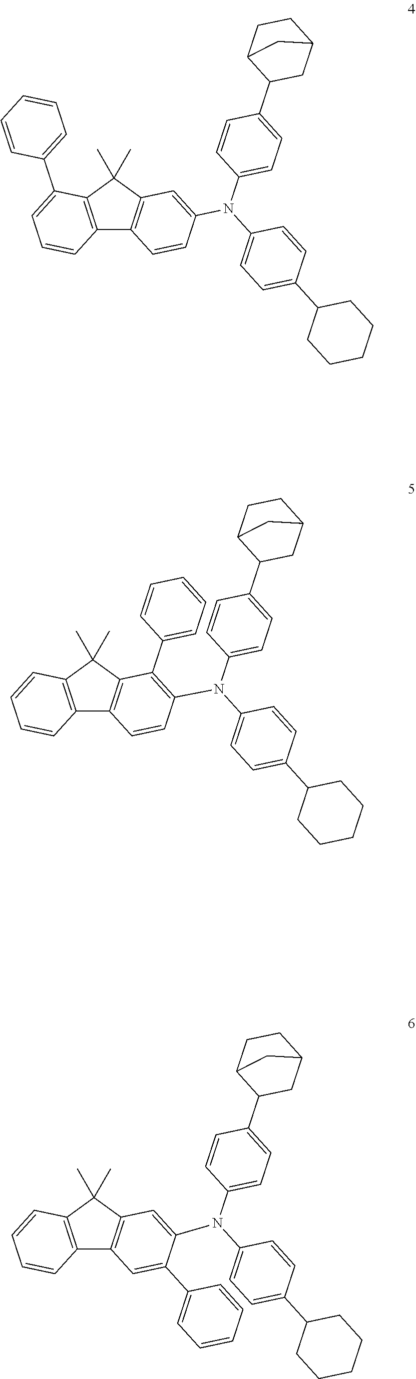 [SMILES: CC1(C)c2cc(N(c3ccc(C4CCCCC4)cc3)c3ccc(C4CC5CCC4C5)cc3)ccc2-c2cccc(-c3ccccc3)c21 ; CC1(C)c2ccccc2-c2ccc(N(c3ccc(C4CCCCC4)cc3)c3ccc(C4CC5CCC4C5)cc3)c(-c3ccccc3)c21 ; CC1(C)c2ccccc2-c2cc(-c3ccccc3)c(N(c3ccc(C4CCCCC4)cc3)c3ccc(C4CC5CCC4C5)cc3)cc21]

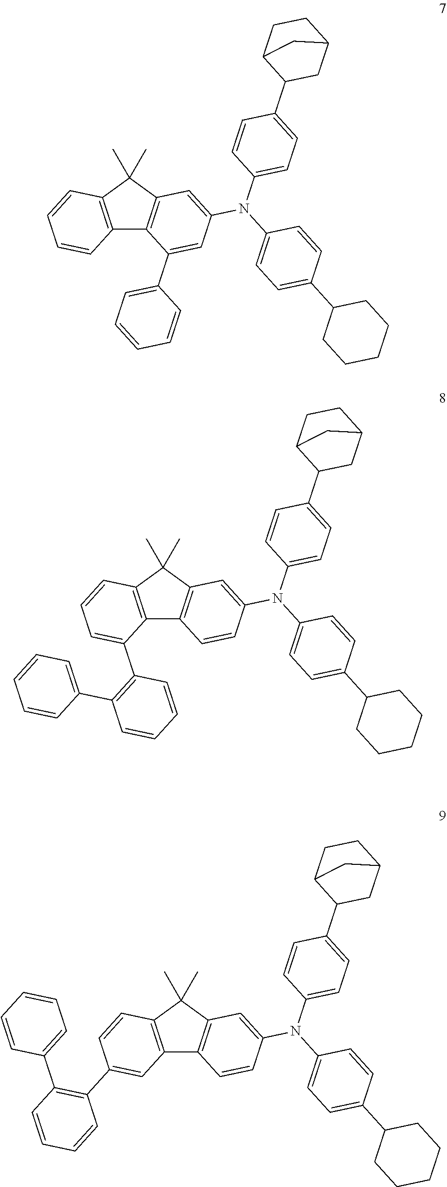

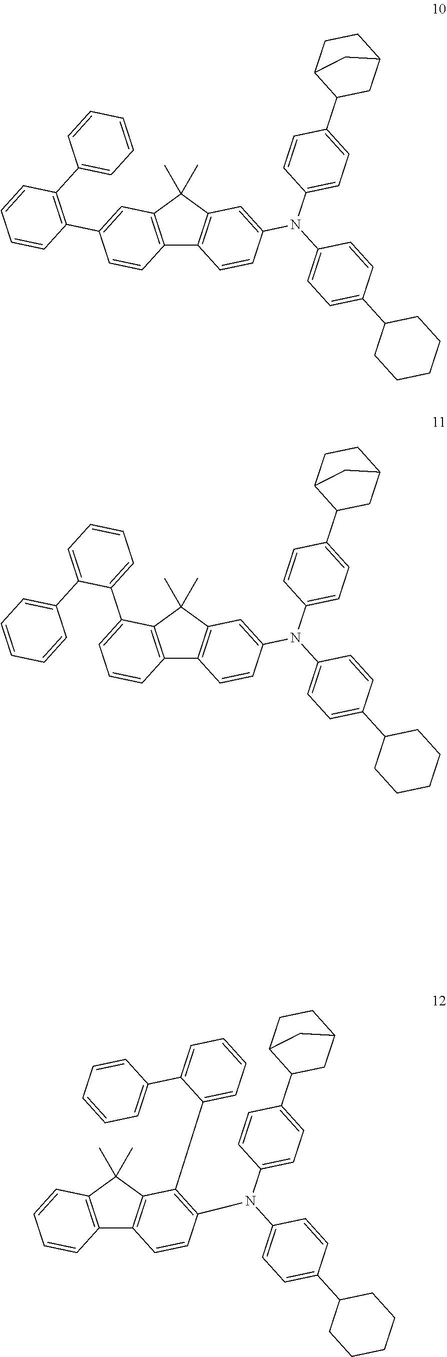 [SMILES: CC1(C)c2cc(-c3ccccc3-c3ccccc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4C5)cc3)c(-c3ccccc3-c3ccccc3)c21]

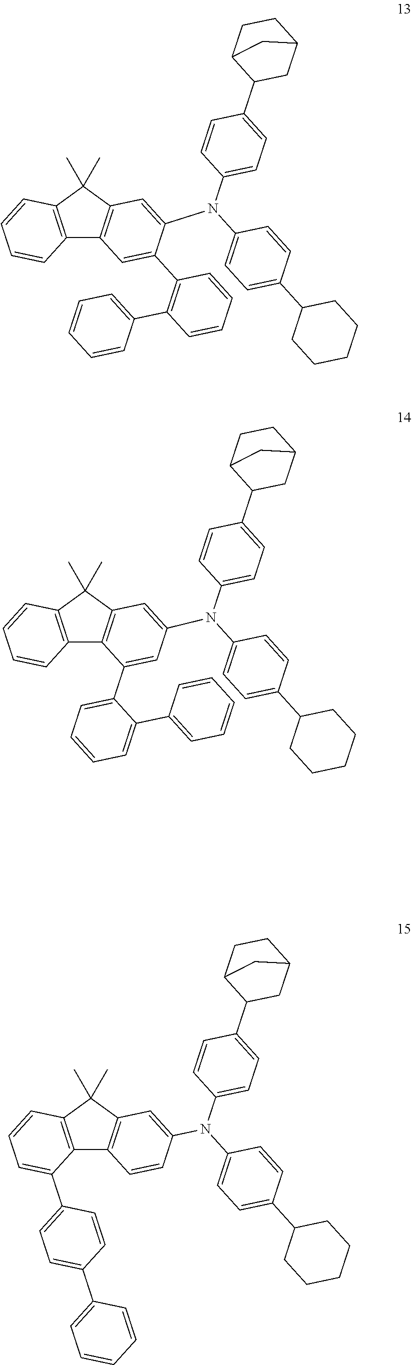 [SMILES: CC1(C)c2ccccc2-c2cc(-c3ccccc3-c3ccccc3)c(N(c3ccc(C4CCCCC4)cc3)c3ccc(C4CC5CCC4C5)cc3)cc21 ; CC1(C)c2ccccc2-c2c(-c3ccccc3-c3ccccc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(-c4ccccc4)cc3)cccc21]

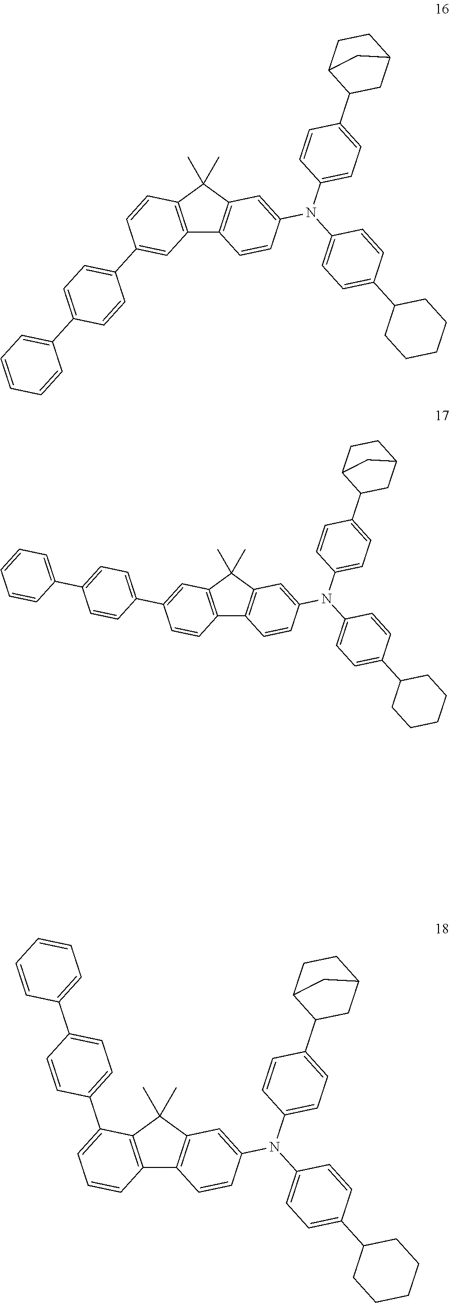 [SMILES: CC1(C)c2ccc(-c3ccc(-c4ccccc4)cc3)cc2-c2ccc(N(c3ccc(C4CCCCC4)cc3)c3ccc(C4CC5CCC4C5)cc3)cc21 ; CC1(C)c2cc(-c3ccc(-c4ccccc4)cc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc(-c4ccccc4)cc3)c21]

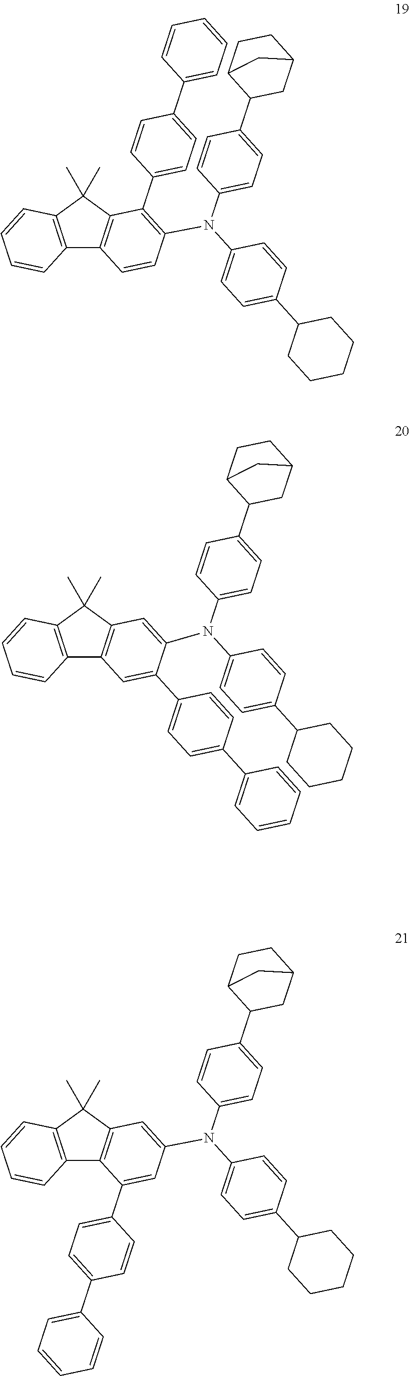 [SMILES: CC1(C)c2ccccc2-c2ccc(N(c3ccc(C4CCCCC4)cc3)c3ccc(C4CC5CCC4C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4CC5CCC4C5)cc3)cc21 ; CC1(C)c2ccccc2-c2c(-c3ccc(-c4ccccc4)cc3)cc(N(c3ccc(C4CCCCC4)cc3)c3ccc(C4CC5CCC4C5)cc3)cc21]

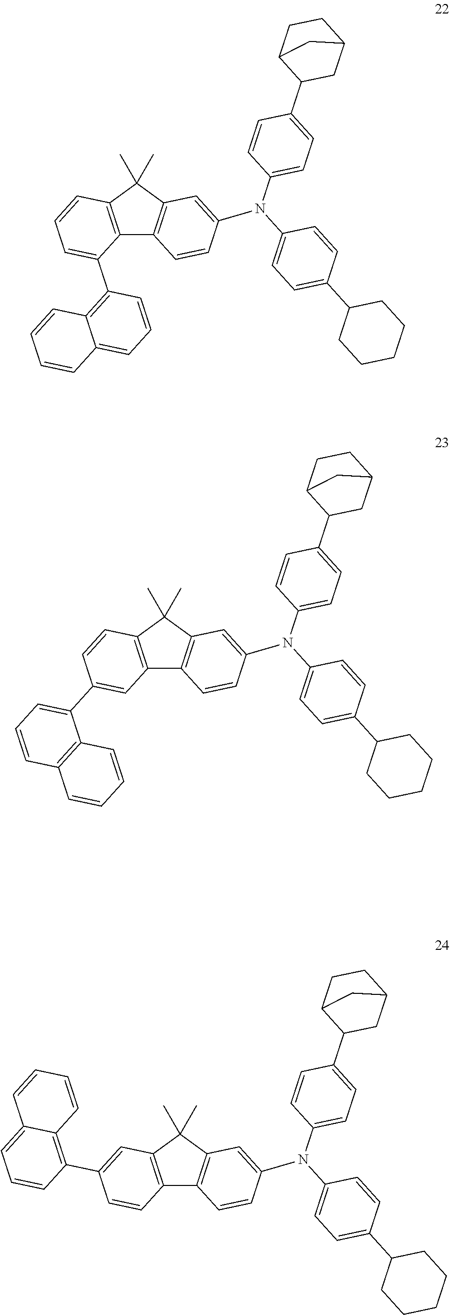 [SMILES: CC1(C)c2cc(N(c3ccc(C4CCCCC4)cc3)c3ccc(C4CC5CCC4C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CC5CCC4C5)cc3)cc21 ; CC1(C)c2cc(-c3cccc4ccccc34)ccc2-c2ccc(N(c3ccc(C4CCCCC4)cc3)c3ccc(C4CC5CCC4C5)cc3)cc21]

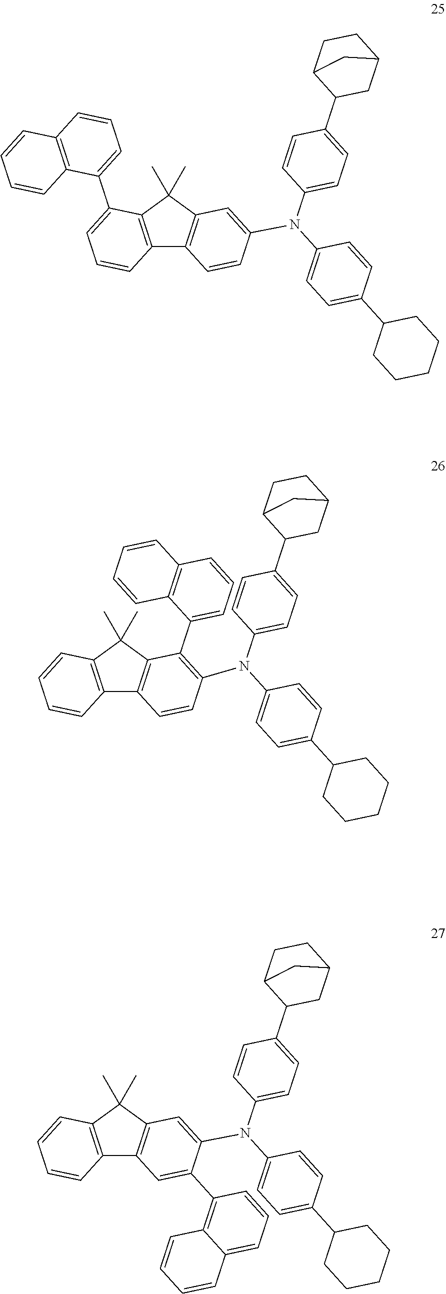 [SMILES: CC1(C)c2cc(N(c3ccc(C4CCCCC4)cc3)c3ccc(C4CC5CCC4C5)cc3)ccc2-c2cccc(-c3cccc4ccccc34)c21 ; CC1(C)c2ccccc2-c2ccc(N(c3ccc(C4CCCCC4)cc3)c3ccc(C4CC5CCC4C5)cc3)c(-c3cccc4ccccc34)c21 ; CC1(C)c2ccccc2-c2cc(-c3cccc4ccccc34)c(N(c3ccc(C4CCCCC4)cc3)c3ccc(C4CC5CCC4C5)cc3)cc21]

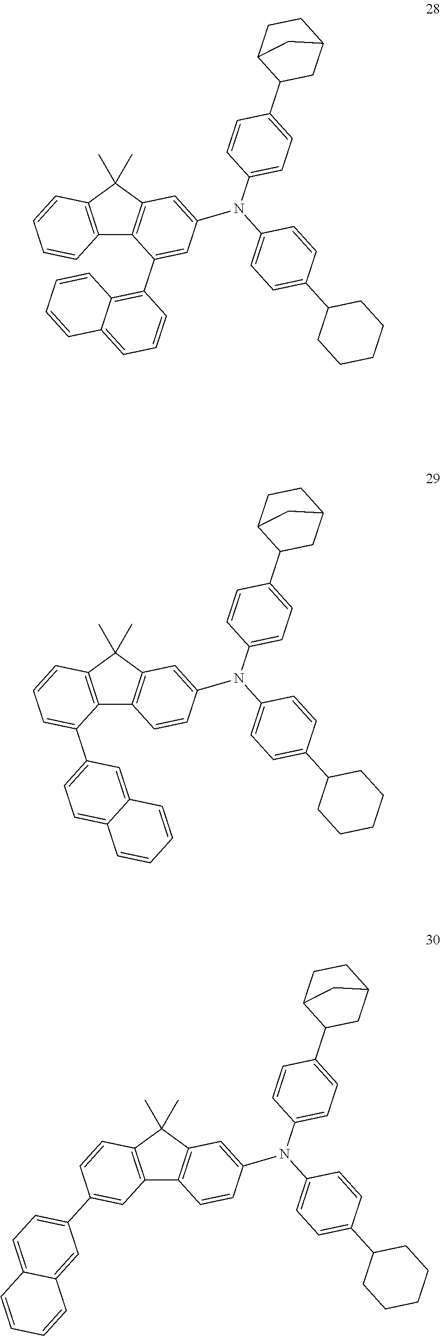 [SMILES: CC1(C)c2ccccc2-c2c(-c3cccc4ccccc34)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CC5CCC4C5)cc3)cc21]

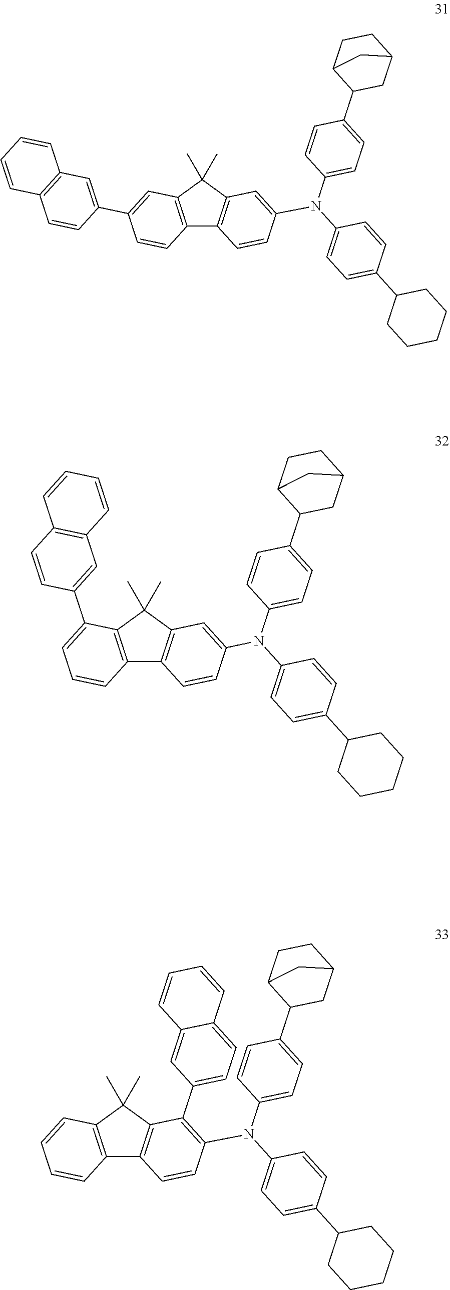 [SMILES: CC1(C)c2cc(-c3ccc4ccccc4c3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc4ccccc4c3)c21 ; CC1(C)c2ccccc2-c2ccc(N(c3ccc(C4CCCCC4)cc3)c3ccc(C4CC5CCC4C5)cc3)c(-c3ccc4ccccc4c3)c21]

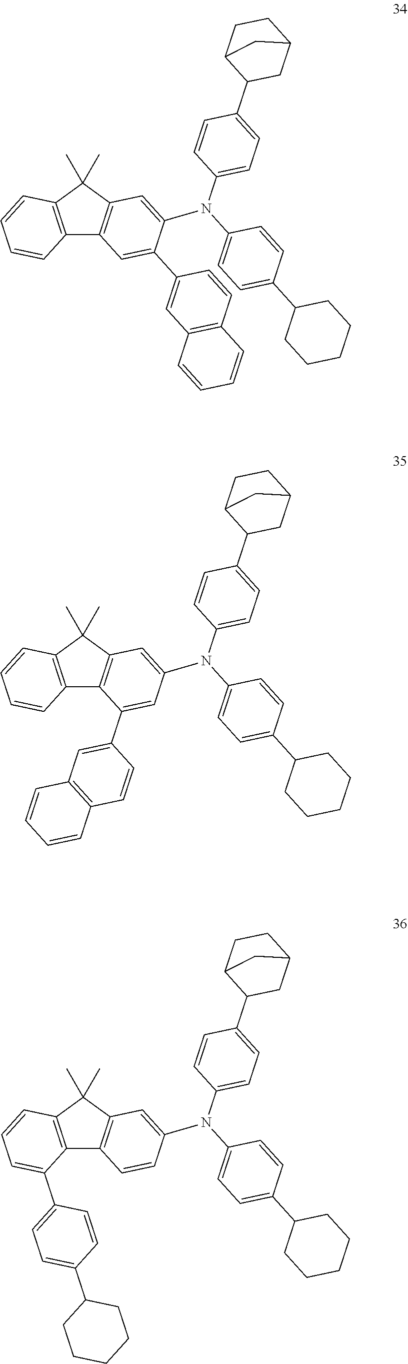 [SMILES: CC1(C)c2ccccc2-c2cc(-c3ccc4ccccc4c3)c(N(c3ccc(C4CCCCC4)cc3)c3ccc(C4CC5CCC4C5)cc3)cc21 ; CC1(C)c2ccccc2-c2c(-c3ccc4ccccc4c3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(C4CCCCC4)cc3)cccc21]

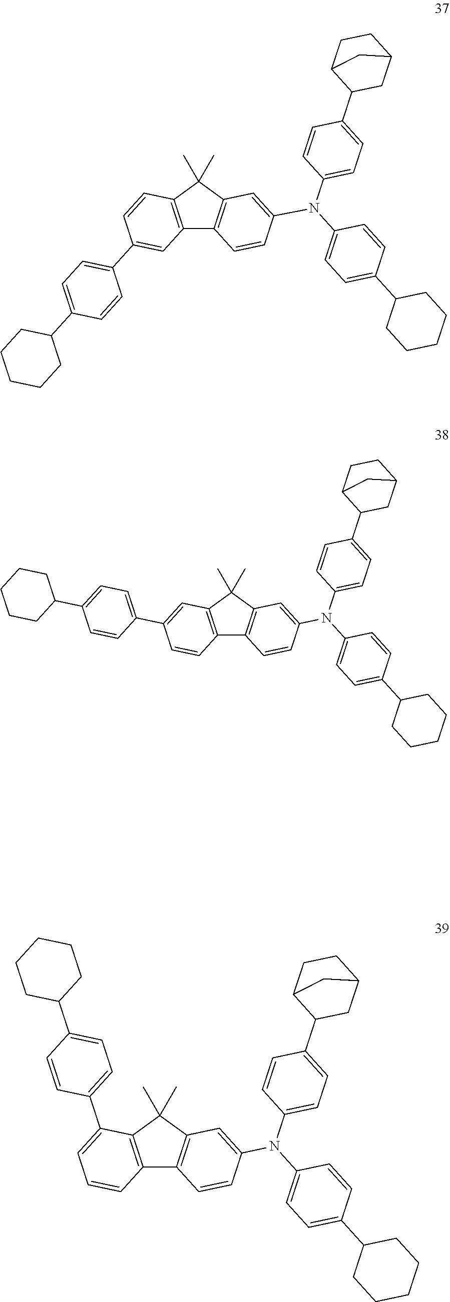 [SMILES: CC1(C)c2ccc(-c3ccc(C4CCCCC4)cc3)cc2-c2ccc(N(c3ccc(C4CCCCC4)cc3)c3ccc(C4CC5CCC4C5)cc3)cc21 ; CC1(C)c2cc(-c3ccc(C4CCCCC4)cc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc(C4CCCCC4)cc3)c21]

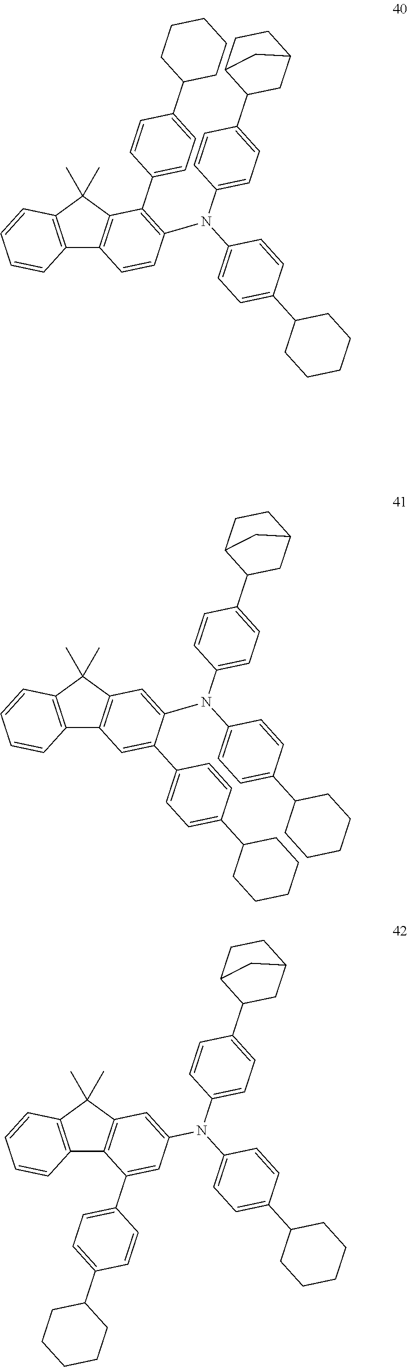

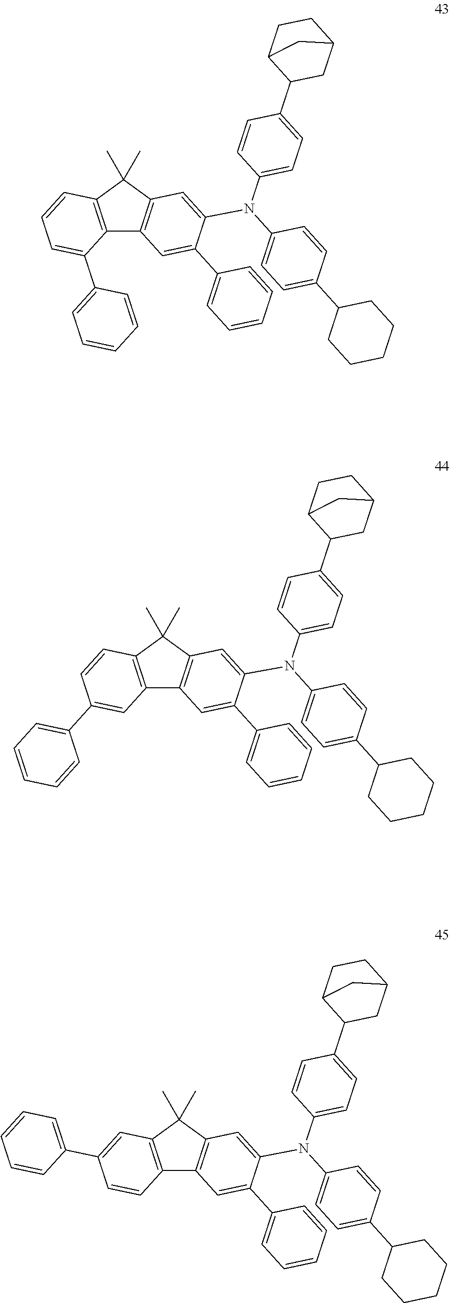 [SMILES: CC1(C)c2cc(N(c3ccc(C4CCCCC4)cc3)c3ccc(C4CC5CCC4C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4CC5CCC4C5)cc3)cc21 ; CC1(C)c2cc(-c3ccccc3)ccc2-c2cc(-c3ccccc3)c(N(c3ccc(C4CCCCC4)cc3)c3ccc(C4CC5CCC4C5)cc3)cc21]

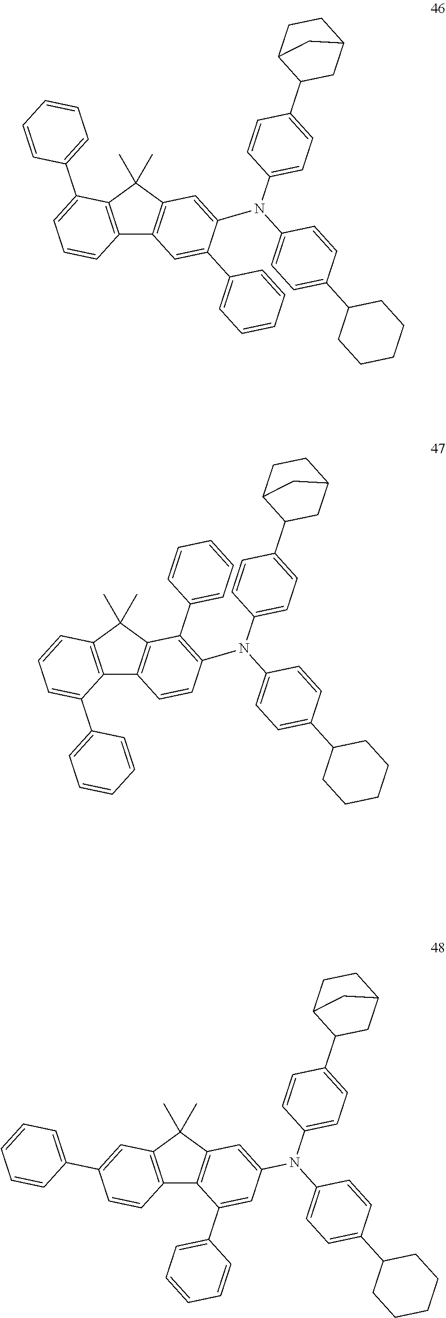 [SMILES: CC1(C)c2cc(N(c3ccc(C4CCCCC4)cc3)c3ccc(C4CC5CCC4C5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4CC5CCC4C5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4CC5CCC4C5)cc3)cc21]

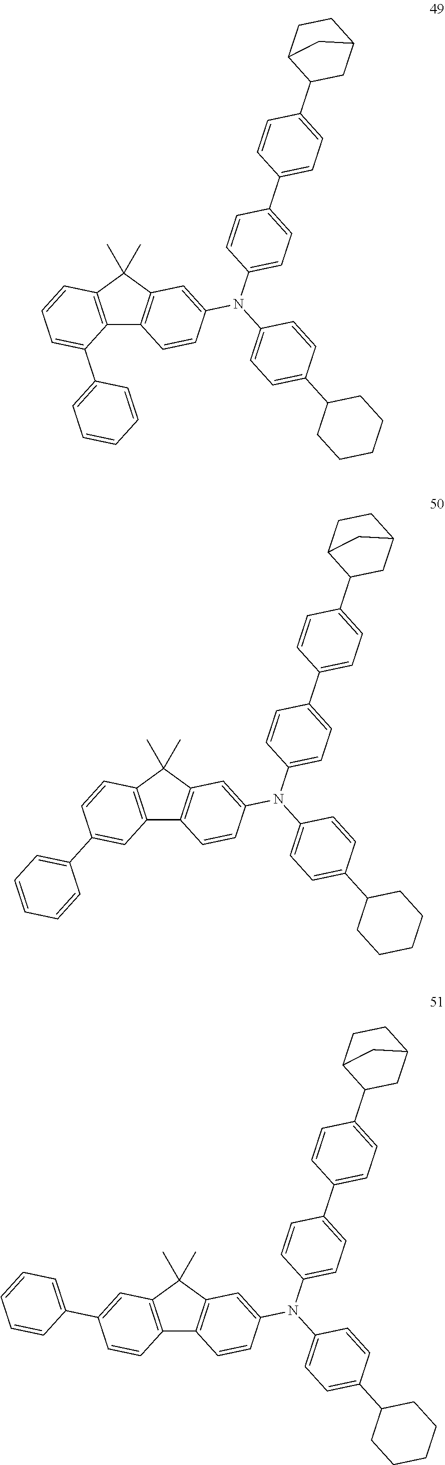 [SMILES: CC1(C)c2cc(N(c3ccc(-c4ccc(C5CC6CCC5C6)cc4)cc3)c3ccc(C4CCCCC4)cc3)ccc2-c2c(-c3ccccc3)cccc21 ; CC1(C)c2ccc(-c3ccccc3)cc2-c2ccc(N(c3ccc(-c4ccc(C5CC6CCC5C6)cc4)cc3)c3ccc(C4CCCCC4)cc3)cc21 ; CC1(C)c2cc(-c3ccccc3)ccc2-c2ccc(N(c3ccc(-c4ccc(C5CC6CCC5C6)cc4)cc3)c3ccc(C4CCCCC4)cc3)cc21]

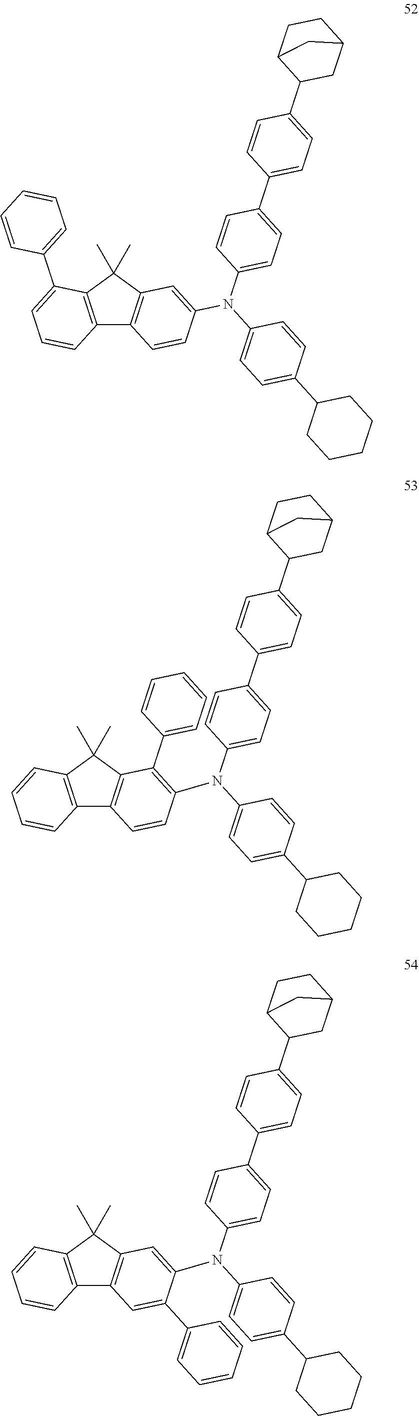

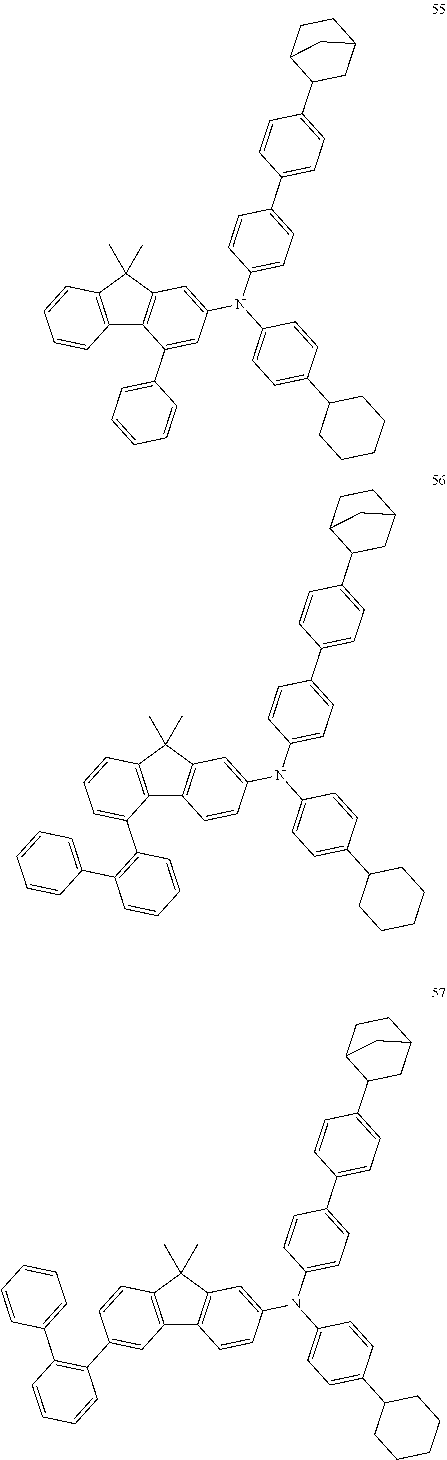

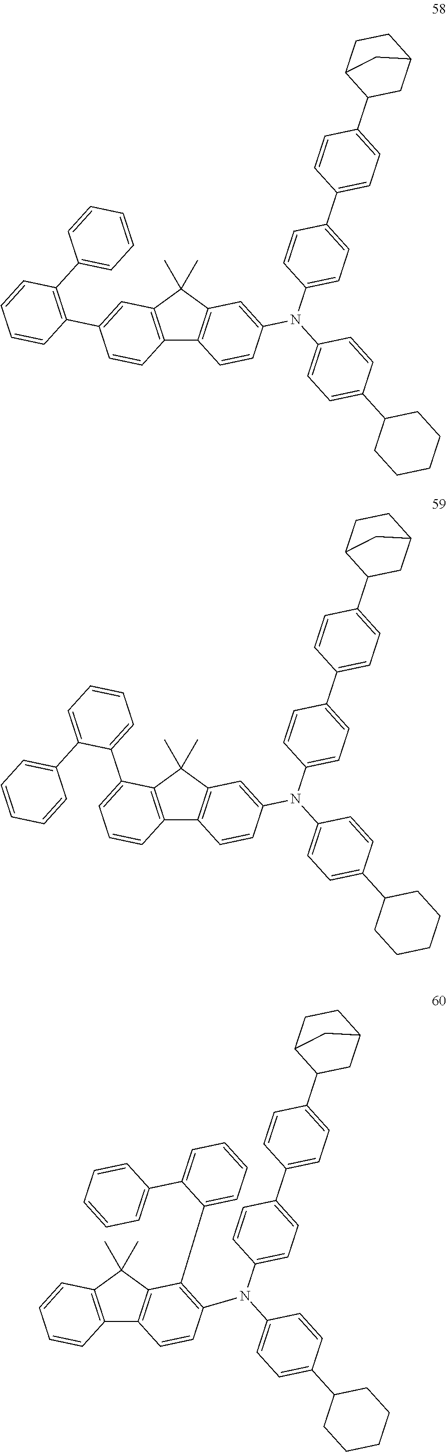

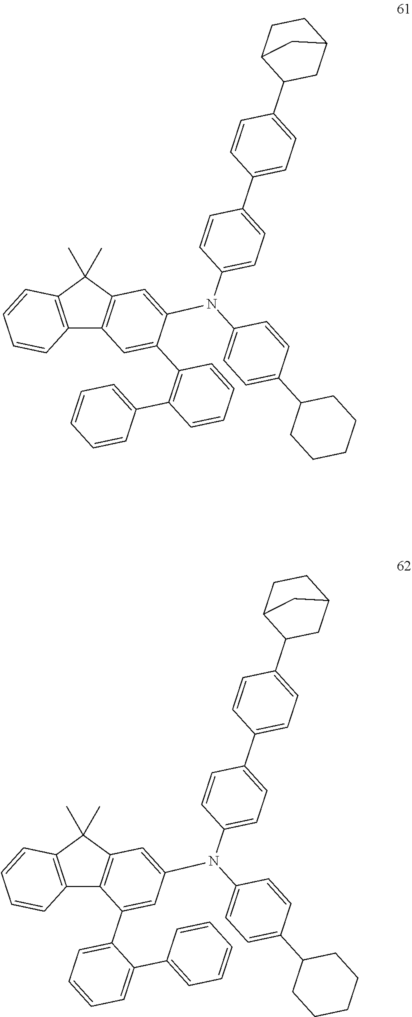 [SMILES: CC1(C)c2ccccc2-c2cc(-c3ccccc3-c3ccccc3)c(N(c3ccc(-c4ccc(C5CC6CCC5C6)cc4)cc3)c3ccc(C4CCCCC4)cc3)cc21 ; CC1(C)c2ccccc2-c2c(-c3ccccc3-c3ccccc3)cc(N(c3ccc(-c4ccc(C5CC6CCC5C6)cc4)cc3)c3ccc(C4CCCCC4)cc3)cc21]

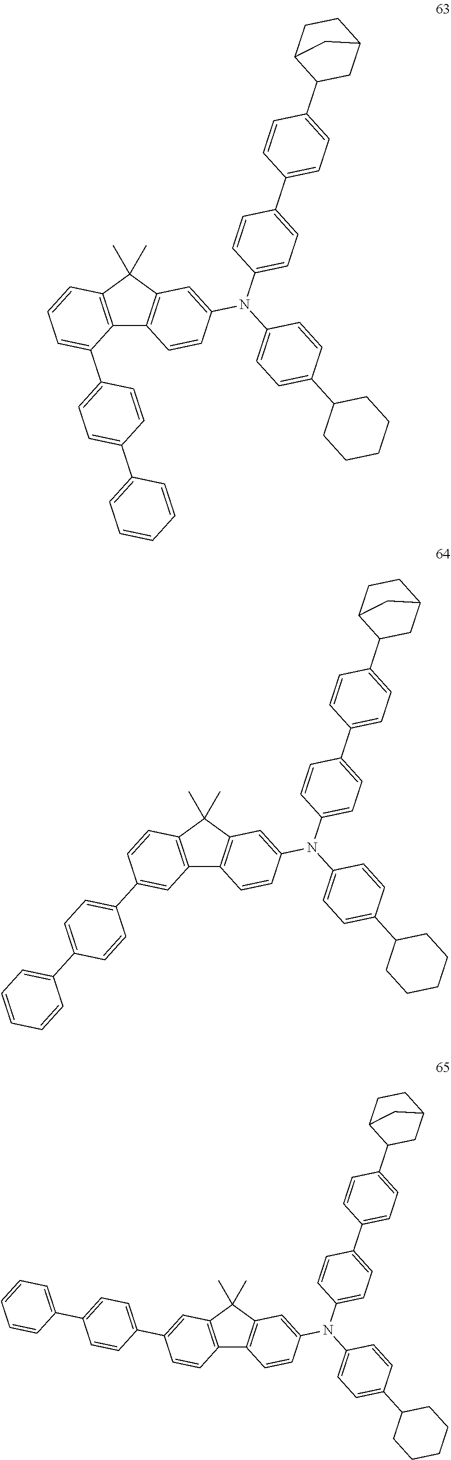 [SMILES: CC1(C)c2cc(N(c3ccc(-c4ccc(C5CC6CCC5C6)cc4)cc3)c3ccc(C4CCCCC4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(-c4ccc(C5CC6CCC5C6)cc4)cc3)c3ccc(C4CCCCC4)cc3)cc21 ; CC1(C)c2cc(-c3ccc(-c4ccccc4)cc3)ccc2-c2ccc(N(c3ccc(-c4ccc(C5CC6CCC5C6)cc4)cc3)c3ccc(C4CCCCC4)cc3)cc21]

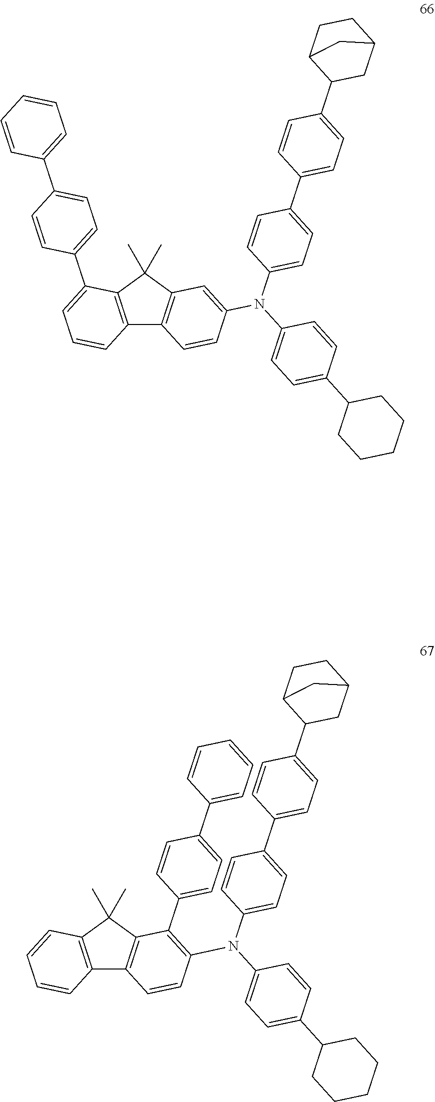 [SMILES: CC1(C)c2cc(N(c3ccc(-c4ccc(C5CC6CCC5C6)cc4)cc3)c3ccc(C4CCCCC4)cc3)ccc2-c2cccc(-c3ccc(-c4ccccc4)cc3)c21 ; CC1(C)c2ccccc2-c2ccc(N(c3ccc(-c4ccc(C5CC6CCC5C6)cc4)cc3)c3ccc(C4CCCCC4)cc3)c(-c3ccc(-c4ccccc4)cc3)c21]

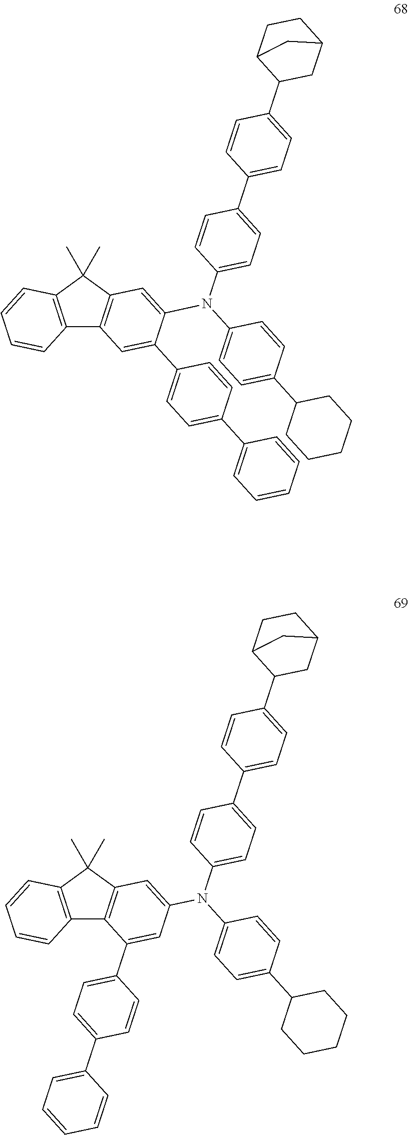 [SMILES: CC1(C)c2ccccc2-c2cc(-c3ccc(-c4ccccc4)cc3)c(N(c3ccc(-c4ccc(C5CC6CCC5C6)cc4)cc3)c3ccc(C4CCCCC4)cc3)cc21 ; CC1(C)c2ccccc2-c2c(-c3ccc(-c4ccccc4)cc3)cc(N(c3ccc(-c4ccc(C5CC6CCC5C6)cc4)cc3)c3ccc(C4CCCCC4)cc3)cc21]

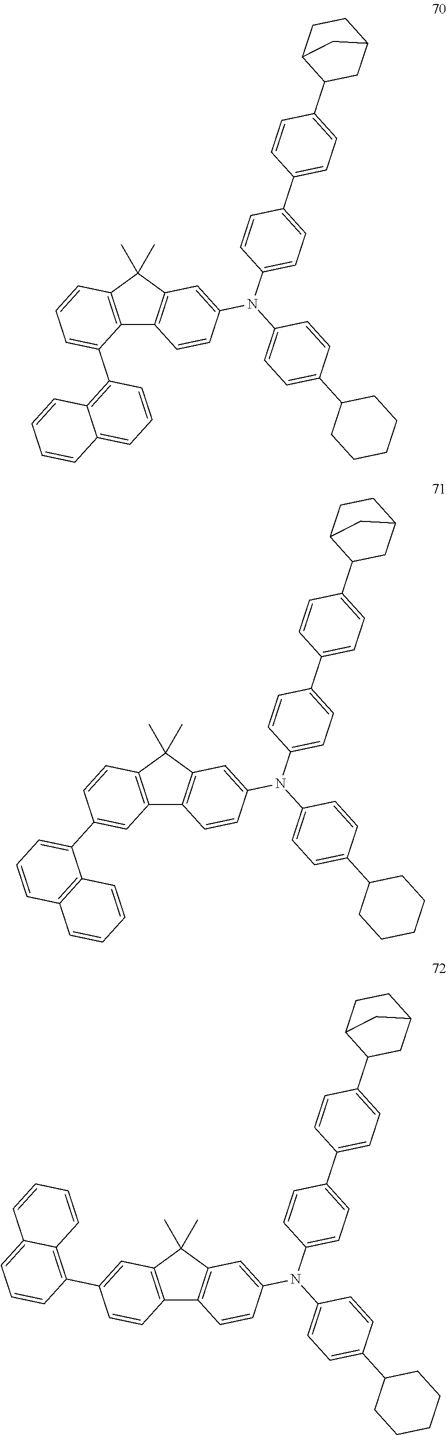

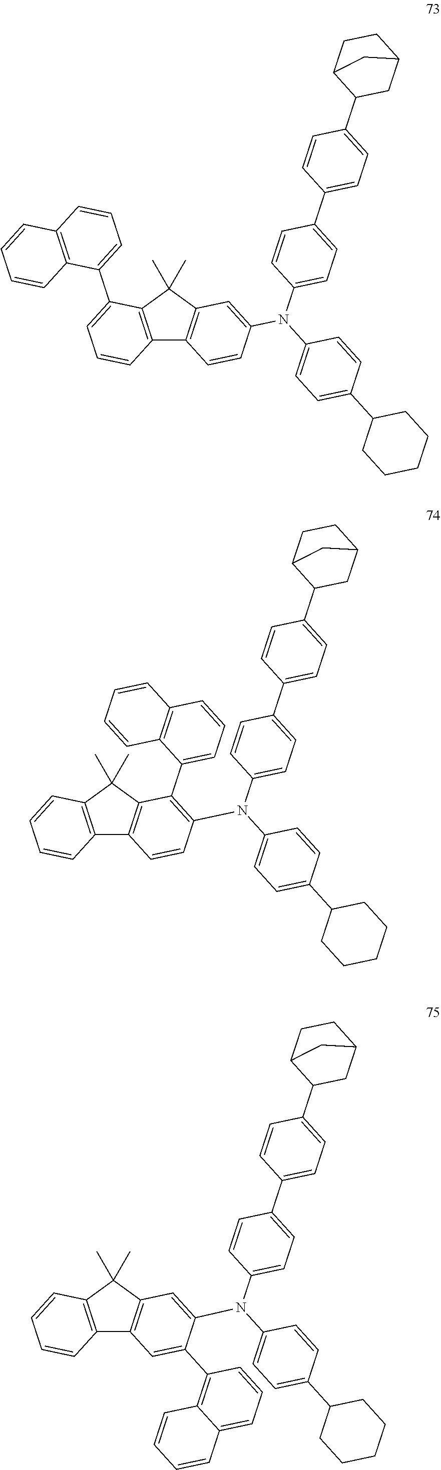 [SMILES: CC1(C)c2cc(N(c3ccc(-c4ccc(C5CC6CCC5C6)cc4)cc3)c3ccc(C4CCCCC4)cc3)ccc2-c2cccc(-c3cccc4ccccc34)c21 ; CC1(C)c2ccccc2-c2ccc(N(c3ccc(-c4ccc(C5CC6CCC5C6)cc4)cc3)c3ccc(C4CCCCC4)cc3)c(-c3cccc4ccccc34)c21 ; CC1(C)c2ccccc2-c2cc(-c3cccc4ccccc34)c(N(c3ccc(-c4ccc(C5CC6CCC5C6)cc4)cc3)c3ccc(C4CCCCC4)cc3)cc21]

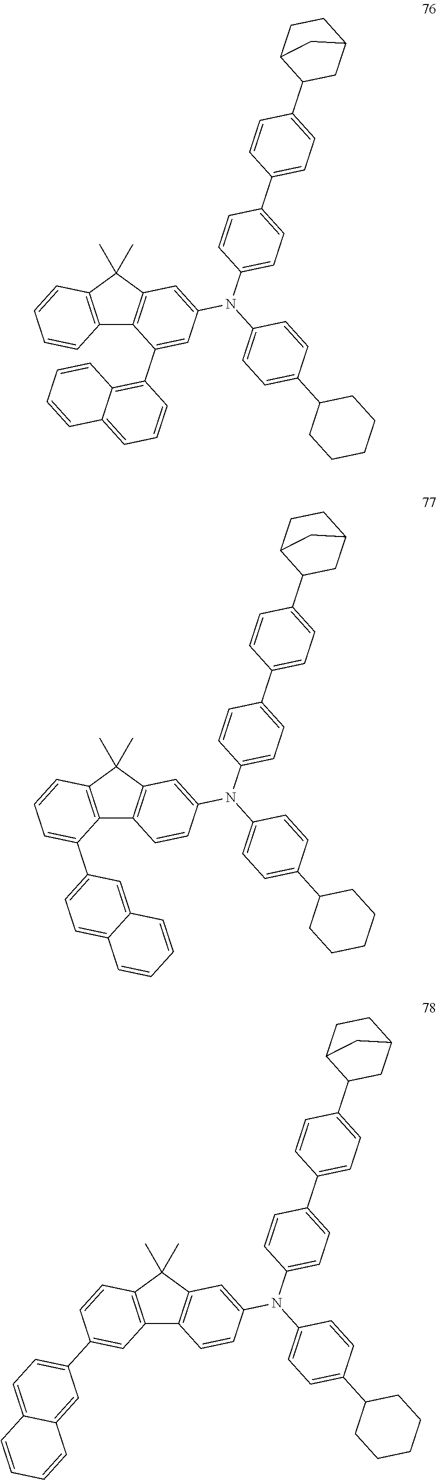 [SMILES: CC1(C)c2ccccc2-c2c(-c3cccc4ccccc34)cc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2c(-c3ccc4ccccc4c3)cccc21 ; CC1(C)c2ccc(-c3ccc4ccccc4c3)cc2-c2ccc(N(c3ccc(-c4ccc(C5CC6CCC5C6)cc4)cc3)c3ccc(C4CCCCC4)cc3)cc21]

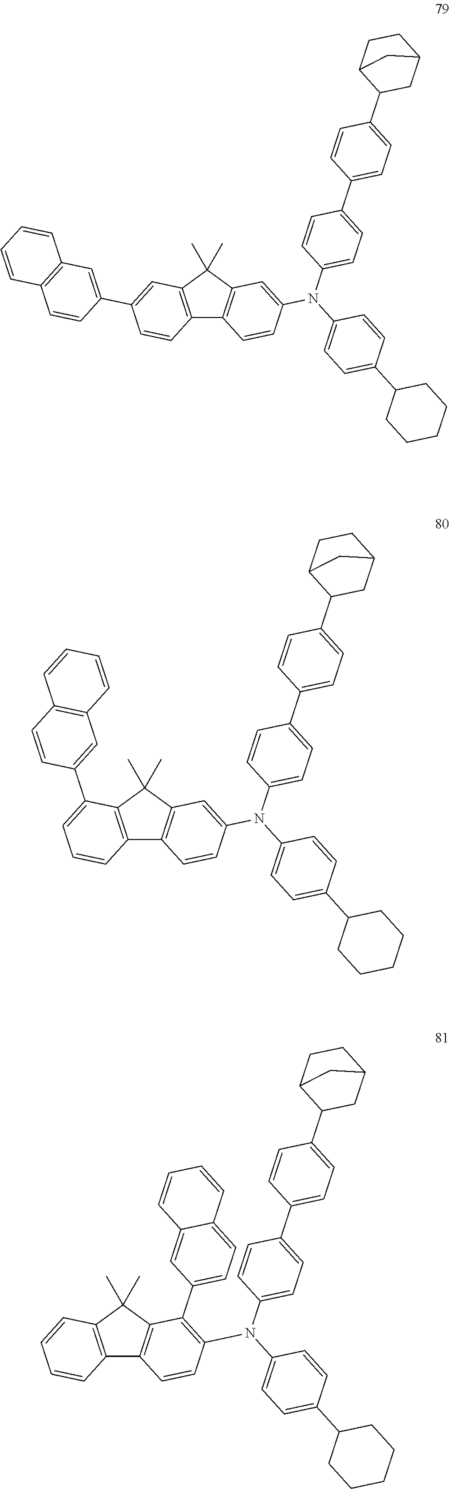 [SMILES: CC1(C)c2cc(-c3ccc4ccccc4c3)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3ccc4ccccc4c3)c21 ; CC1(C)c2ccccc2-c2ccc(N(c3ccc(-c4ccc(C5CC6CCC5C6)cc4)cc3)c3ccc(C4CCCCC4)cc3)c(-c3ccc4ccccc4c3)c21]

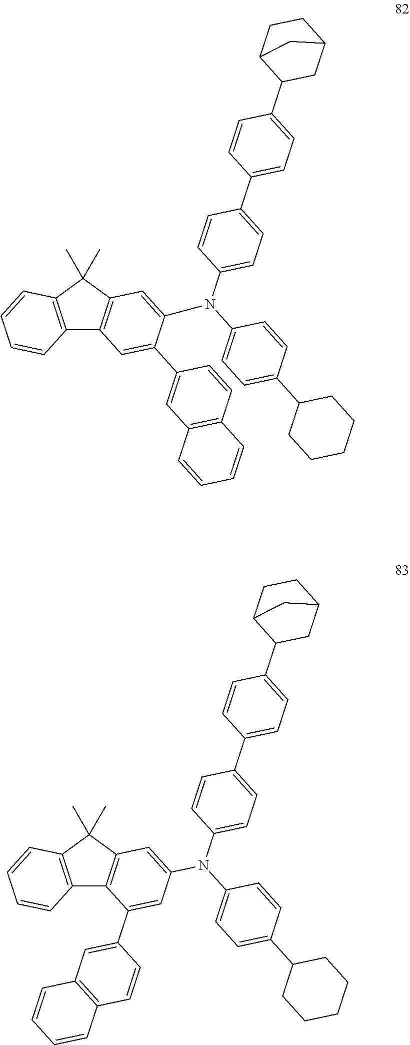 [SMILES: CC1(C)c2ccccc2-c2cc(-c3ccc4ccccc4c3)c(N(c3ccc(-c4ccc(C5CC6CCC5C6)cc4)cc3)c3ccc(C4CCCCC4)cc3)cc21 ; CC1(C)c2ccccc2-c2c(-c3ccc4ccccc4c3)cc(N(c3ccc(-c4ccc(C5CC6CCC5C6)cc4)cc3)c3ccc(C4CCCCC4)cc3)cc21]

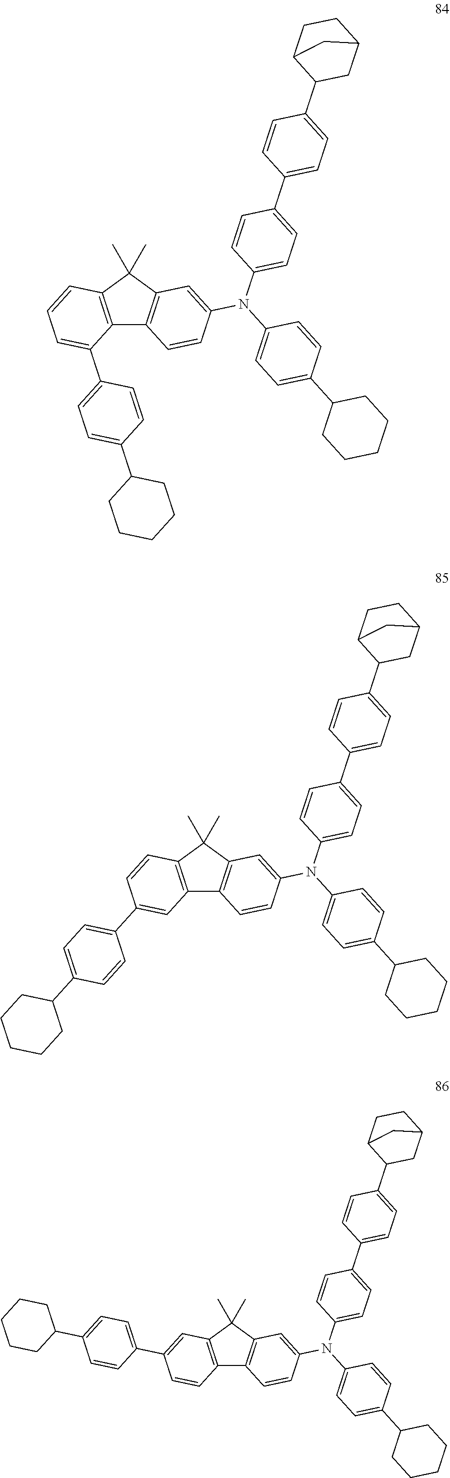 [SMILES: CC1(C)c2cc(N(c3ccc(-c4ccc(C5CC6CCC5C6)cc4)cc3)c3ccc(C4CCCCC4)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(-c4ccc(C5CC6CCC5C6)cc4)cc3)c3ccc(C4CCCCC4)cc3)cc21 ; CC1(C)c2cc(-c3ccc(C4CCCCC4)cc3)ccc2-c2ccc(N(c3ccc(-c4ccc(C5CC6CCC5C6)cc4)cc3)c3ccc(C4CCCCC4)cc3)cc21]

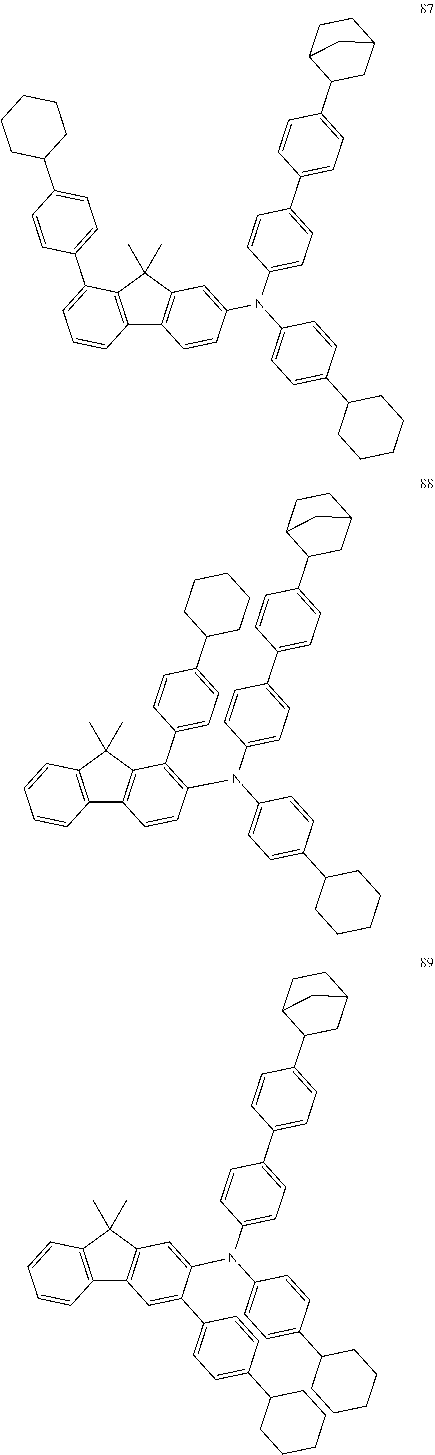

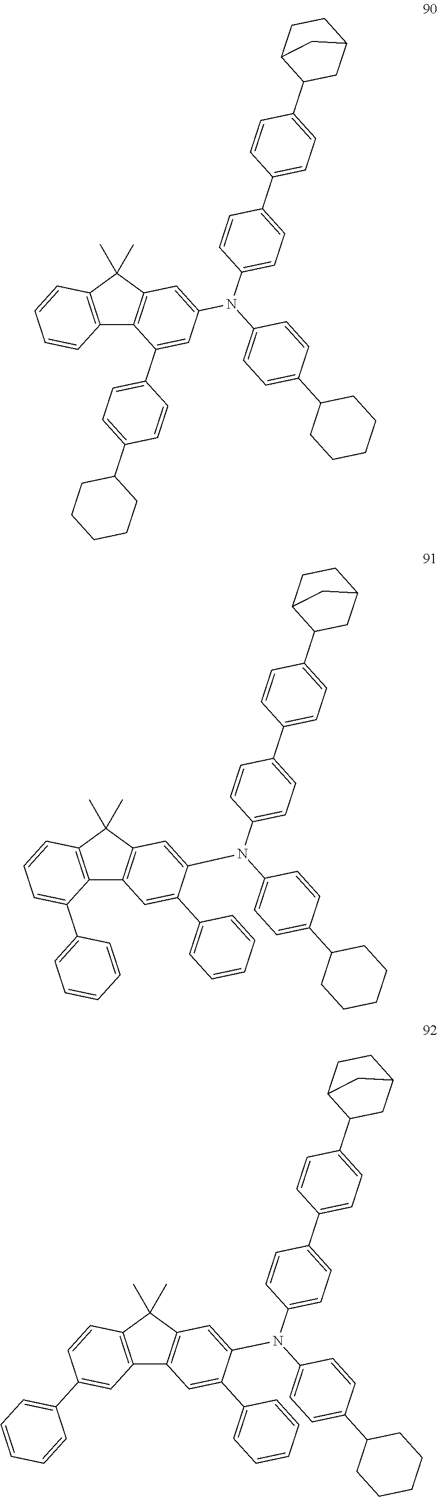 [SMILES: CC1(C)c2ccccc2-c2c(-c3ccc(C4CCCCC4)cc3)cc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)c(-c3ccccc3)cc2-c2c(-c3ccccc3)cccc21 ; CC1(C)c2ccc(-c3ccccc3)cc2-c2cc(-c3ccccc3)c(N(c3ccc(-c4ccc(C5CC6CCC5C6)cc4)cc3)c3ccc(C4CCCCC4)cc3)cc21]

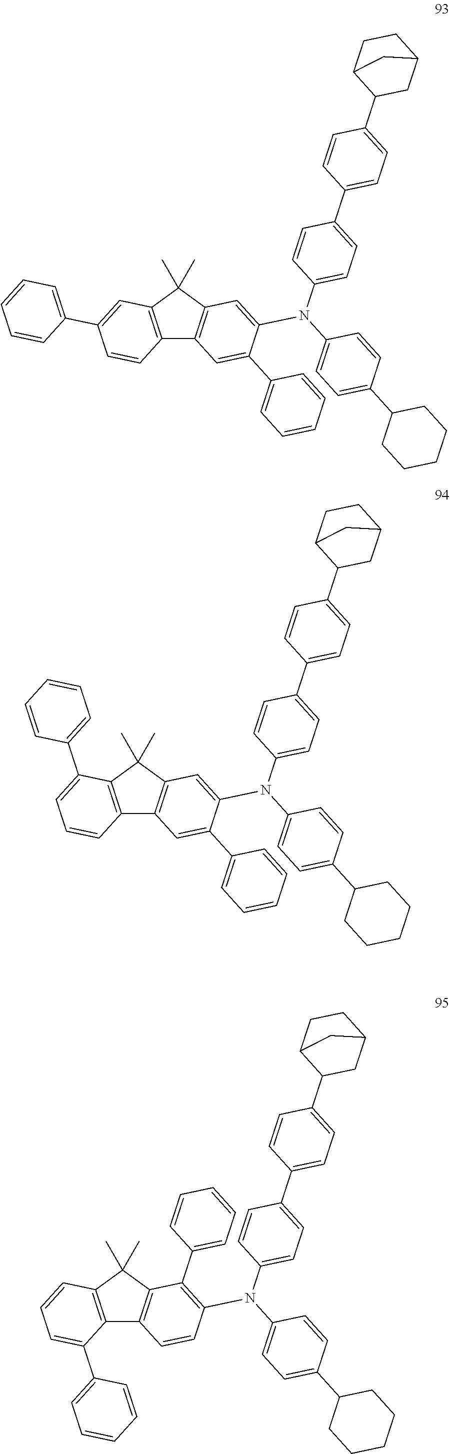 [SMILES: CC1(C)c2cc(-c3ccccc3)ccc2-c2cc(-c3ccccc3)c(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)c(-c3ccccc3)cc2-c2cccc(-c3ccccc3)c21 ; CC1(C)c2cccc(-c3ccccc3)c2-c2ccc(N(c3ccc(-c4ccc(C5CC6CCC5C6)cc4)cc3)c3ccc(C4CCCCC4)cc3)c(-c3ccccc3)c21]

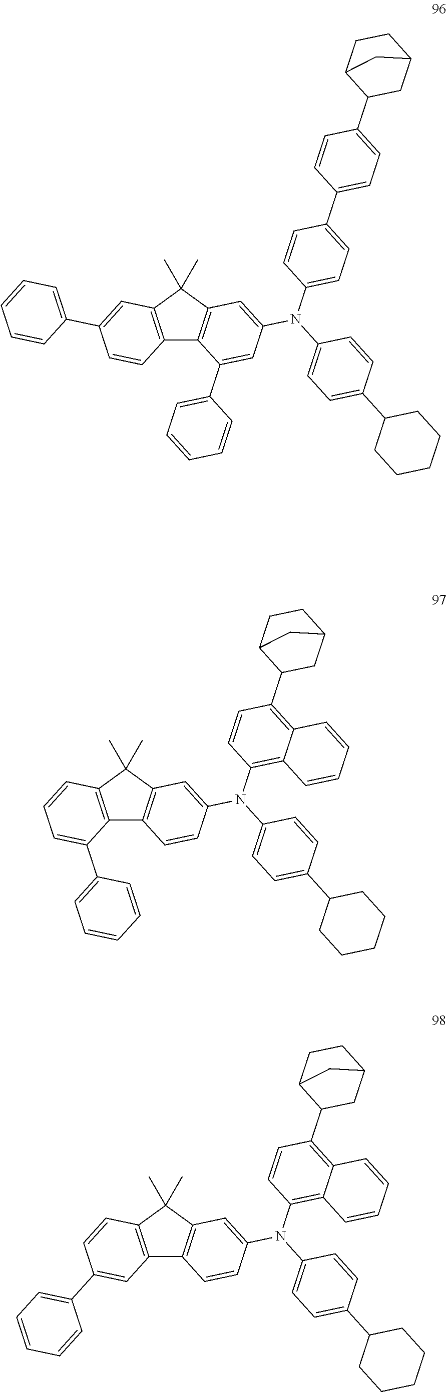 [SMILES: CC1(C)c2cc(-c3ccccc3)ccc2-c2c(-c3ccccc3)cc(N(c3ccc(-c4ccc(C5CC6CCC5C6)cc4)cc3)c3ccc(C4CCCCC4)cc3)cc21 ; CC1(C)c2cc(N(c3ccc(C4CCCCC4)cc3)c3ccc(C4CC5CCC4C5)c4ccccc34)ccc2-c2c(-c3ccccc3)cccc21 ; CC1(C)c2ccc(-c3ccccc3)cc2-c2ccc(N(c3ccc(C4CCCCC4)cc3)c3ccc(C4CC5CCC4C5)c4ccccc34)cc21]

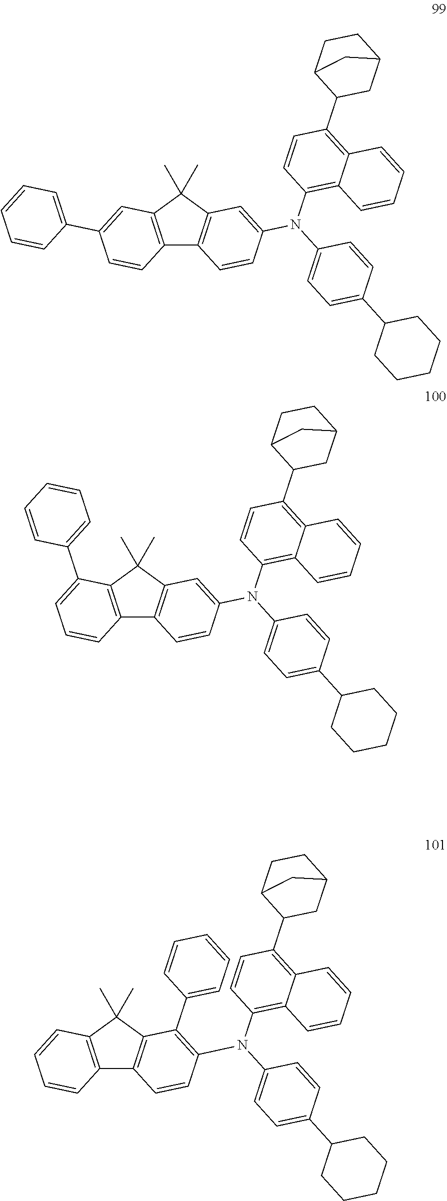 [SMILES: CC1(C)c2cc(-c3ccccc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccccc3)c21 ; CC1(C)c2ccccc2-c2ccc(N(c3ccc(C4CCCCC4)cc3)c3ccc(C4CC5CCC4C5)c4ccccc34)c(-c3ccccc3)c21]

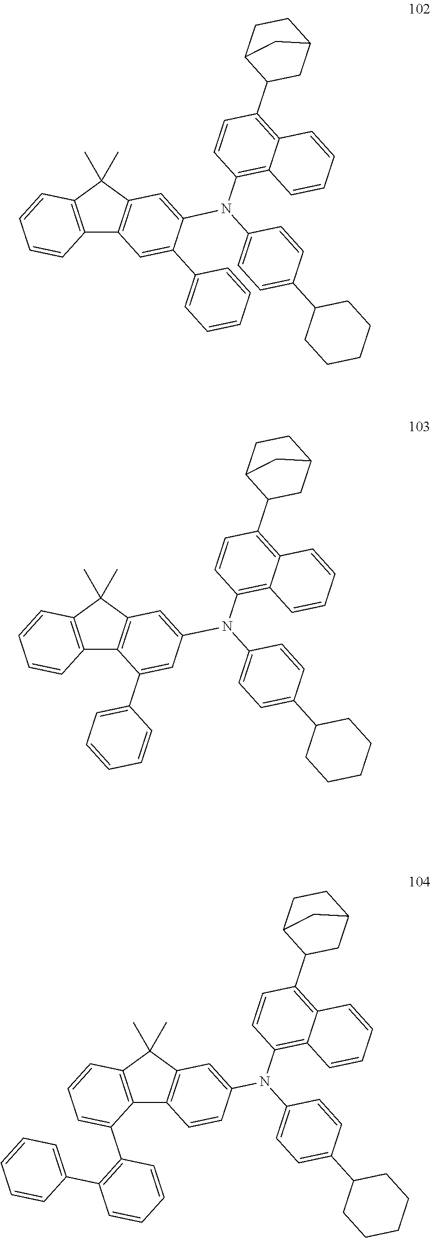 [SMILES: CC1(C)c2ccccc2-c2cc(-c3ccccc3)c(N(c3ccc(C4CCCCC4)cc3)c3ccc(C4CC5CCC4C5)c4ccccc34)cc21 ; CC1(C)c2ccccc2-c2c(-c3ccccc3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccccc3-c3ccccc3)cccc21]

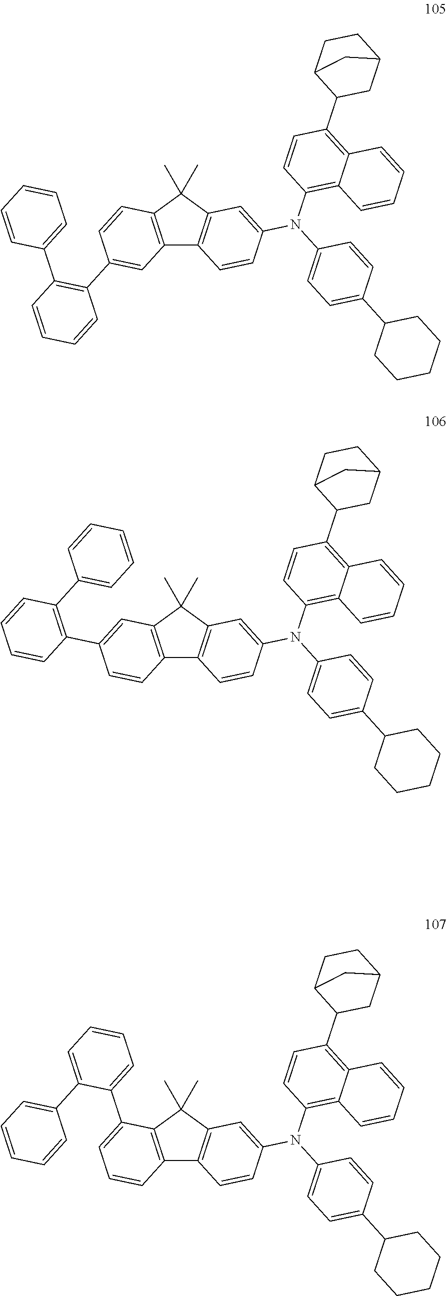 [SMILES: CC1(C)c2ccc(-c3ccccc3-c3ccccc3)cc2-c2ccc(N(c3ccc(C4CCCCC4)cc3)c3ccc(C4CC5CCC4C5)c4ccccc34)cc21 ; CC1(C)c2cc(-c3ccccc3-c3ccccc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccccc3-c3ccccc3)c21]

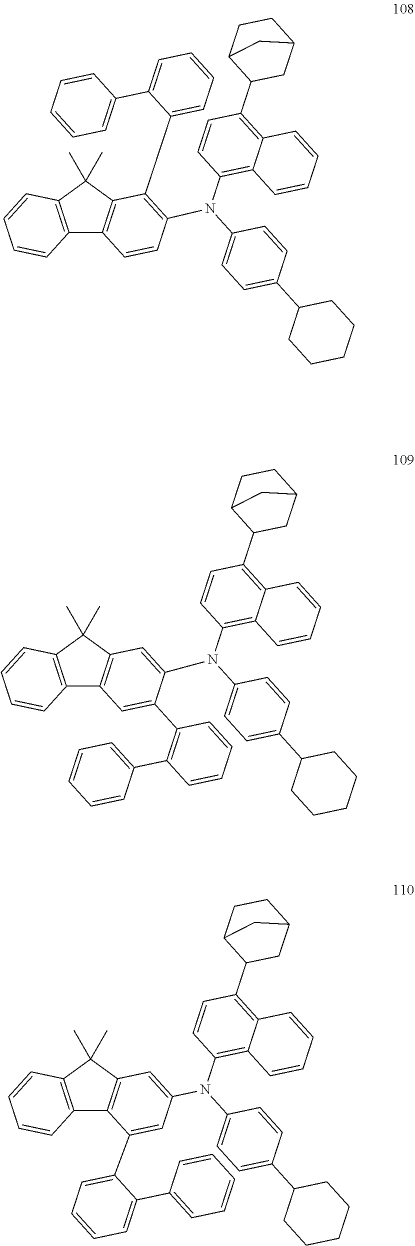 [SMILES: CC1(C)c2ccccc2-c2ccc(N(c3ccc(C4CCCCC4)cc3)c3ccc(C4CC5CCC4C5)c4ccccc34)c(-c3ccccc3-c3ccccc3)c21 ; CC1(C)c2ccccc2-c2cc(-c3ccccc3-c3ccccc3)c(N(c3ccc(C4CCCCC4)cc3)c3ccc(C4CC5CCC4C5)c4ccccc34)cc21 ; CC1(C)c2ccccc2-c2c(-c3ccccc3-c3ccccc3)cc(N(c3ccc(C4CCCCC4)cc3)c3ccc(C4CC5CCC4C5)c4ccccc34)cc21]

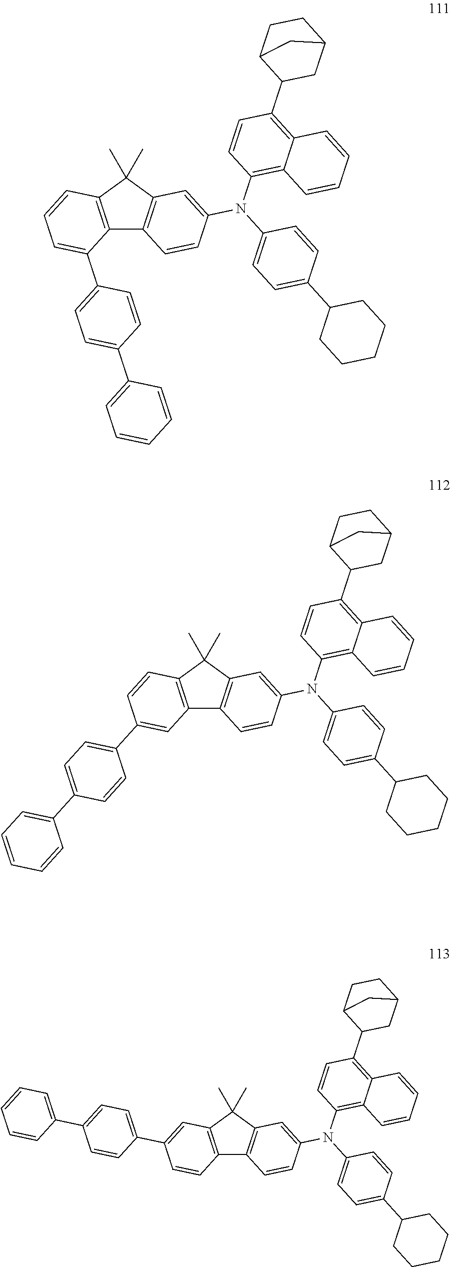 [SMILES: CC1(C)c2cc(N(c3ccc(C4CCCCC4)cc3)c3ccc(C4CC5CCC4C5)c4ccccc34)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4CC5CCC4C5)c4ccccc34)cc21 ; CC1(C)c2cc(-c3ccc(-c4ccccc4)cc3)ccc2-c2ccc(N(c3ccc(C4CCCCC4)cc3)c3ccc(C4CC5CCC4C5)c4ccccc34)cc21]

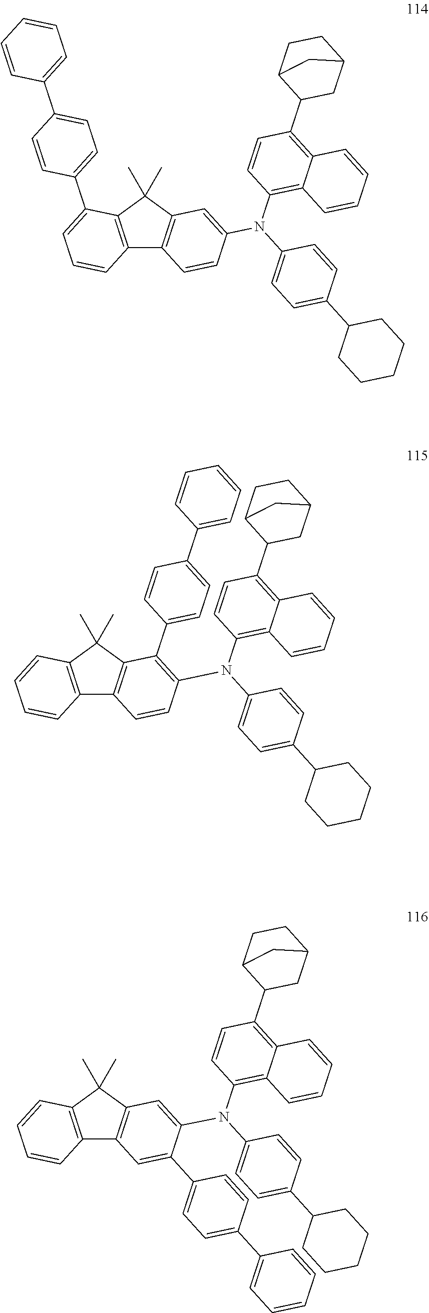 [SMILES: CC1(C)c2cc(N(c3ccc(C4CCCCC4)cc3)c3ccc(C4CC5CCC4C5)c4ccccc34)ccc2-c2cccc(-c3ccc(-c4ccccc4)cc3)c21 ; CC1(C)c2ccccc2-c2ccc(N(c3ccc(C4CCCCC4)cc3)c3ccc(C4CC5CCC4C5)c4ccccc34)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4CC5CCC4C5)c4ccccc34)cc21]

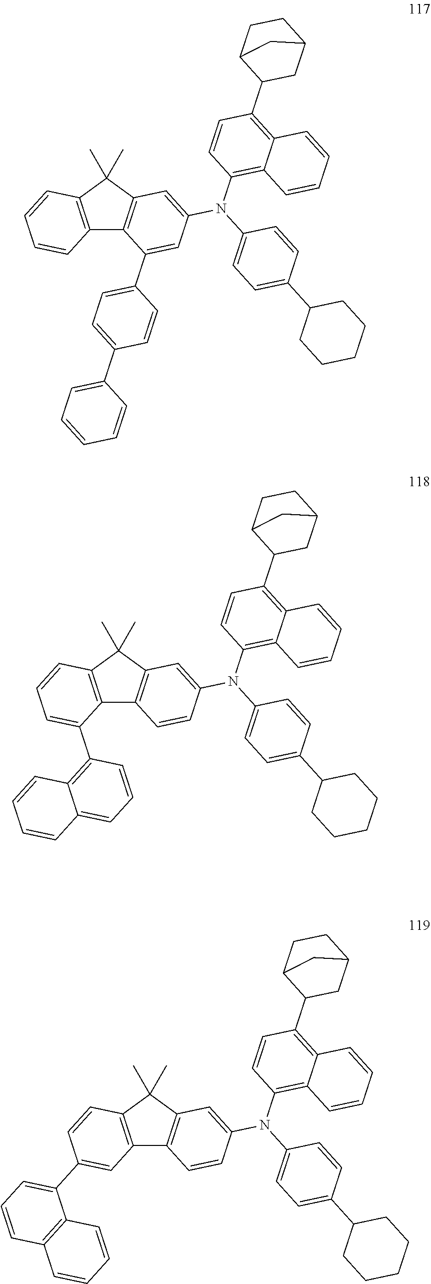 [SMILES: CC1(C)c2ccccc2-c2c(-c3ccc(-c4ccccc4)cc3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CC5CCC4C5)c4ccccc34)cc21]

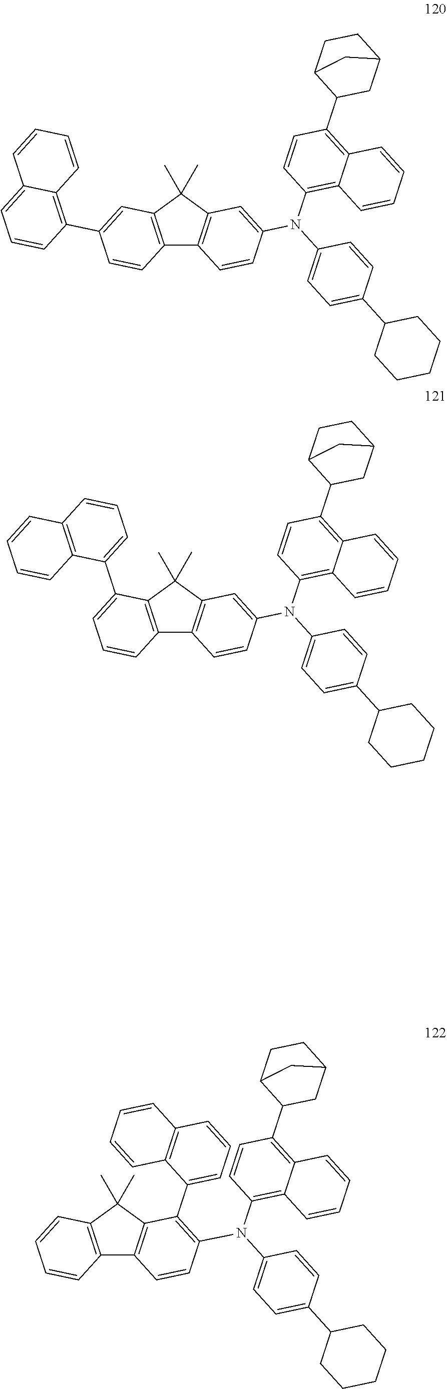 [SMILES: CC1(C)c2cc(-c3cccc4ccccc34)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3cccc4ccccc34)c21 ; CC1(C)c2ccccc2-c2ccc(N(c3ccc(C4CCCCC4)cc3)c3ccc(C4CC5CCC4C5)c4ccccc34)c(-c3cccc4ccccc34)c21]

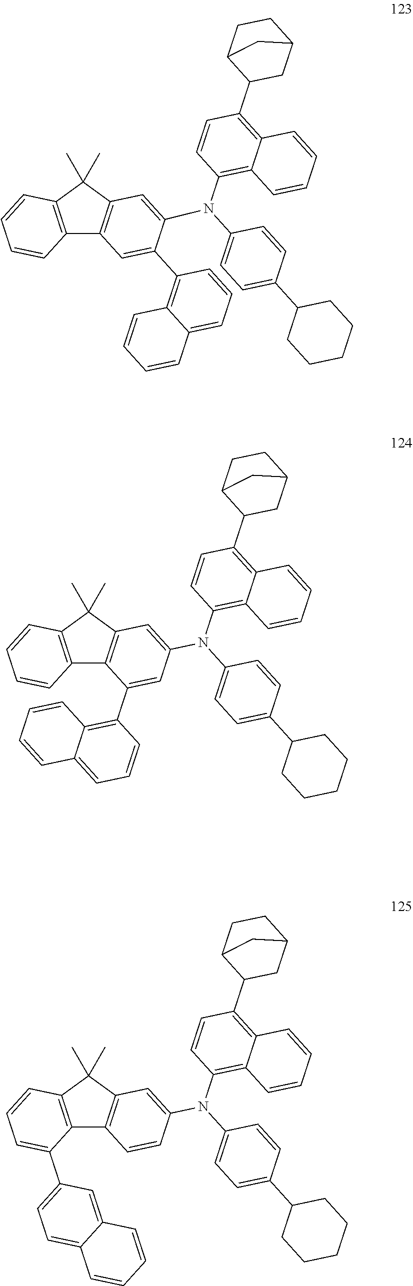 [SMILES: CC1(C)c2ccccc2-c2cc(-c3cccc4ccccc34)c(N(c3ccc(C4CCCCC4)cc3)c3ccc(C4CC5CCC4C5)c4ccccc34)cc21 ; CC1(C)c2ccccc2-c2c(-c3cccc4ccccc34)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc4ccccc4c3)cccc21]

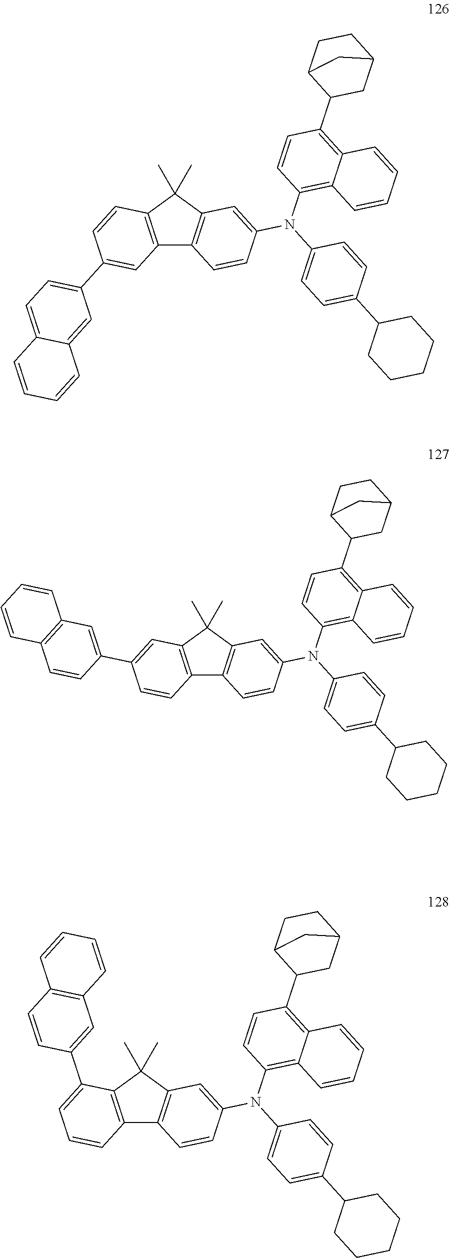 [SMILES: CC1(C)c2ccc(-c3ccc4ccccc4c3)cc2-c2ccc(N(c3ccc(C4CCCCC4)cc3)c3ccc(C4CC5CCC4C5)c4ccccc34)cc21 ; CC1(C)c2cc(-c3ccc4ccccc4c3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc4ccccc4c3)c21]

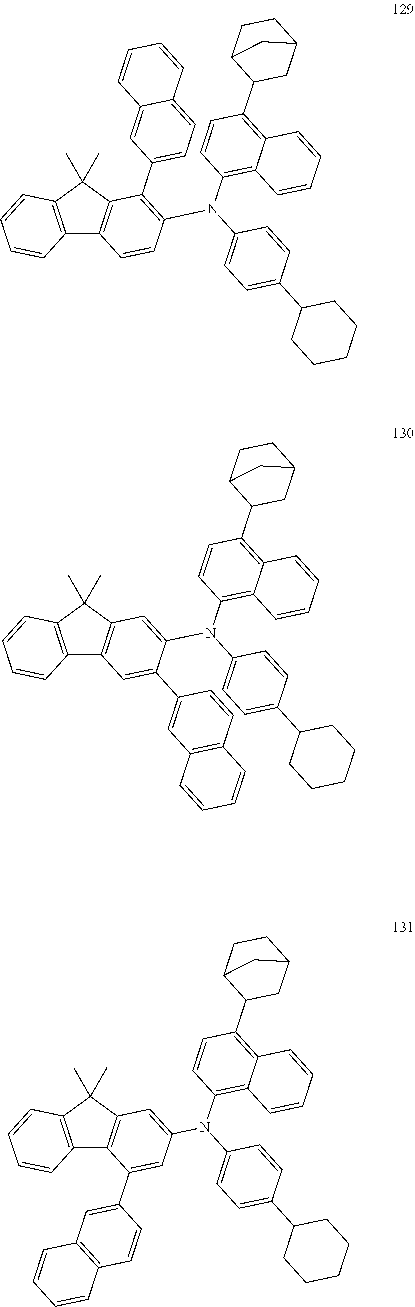 [SMILES: CC1(C)c2ccccc2-c2ccc(N(c3ccc(C4CCCCC4)cc3)c3ccc(C4CC5CCC4C5)c4ccccc34)c(-c3ccc4ccccc4c3)c21 ; CC1(C)c2ccccc2-c2cc(-c3ccc4ccccc4c3)c(N(c3ccc(C4CCCCC4)cc3)c3ccc(C4CC5CCC4C5)c4ccccc34)cc21 ; CC1(C)c2ccccc2-c2c(-c3ccc4ccccc4c3)cc(N(c3ccc(C4CCCCC4)cc3)c3ccc(C4CC5CCC4C5)c4ccccc34)cc21]

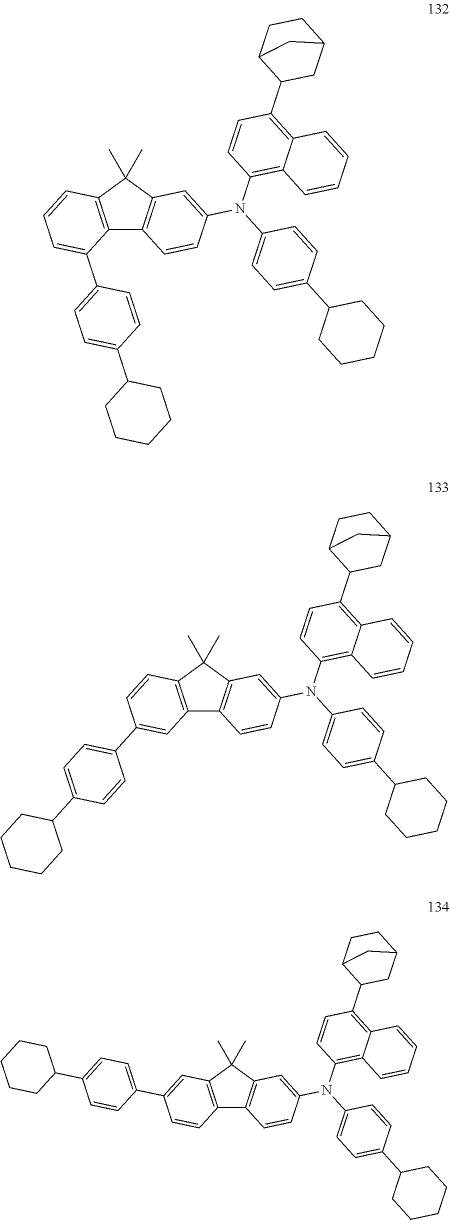 [SMILES: CC1(C)c2cc(N(c3ccc(C4CCCCC4)cc3)c3ccc(C4CC5CCC4C5)c4ccccc34)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CCCCC4)cc3)c3ccc(C4CC5CCC4C5)c4ccccc34)cc21 ; CC1(C)c2cc(-c3ccc(C4CCCCC4)cc3)ccc2-c2ccc(N(c3ccc(C4CCCCC4)cc3)c3ccc(C4CC5CCC4C5)c4ccccc34)cc21]

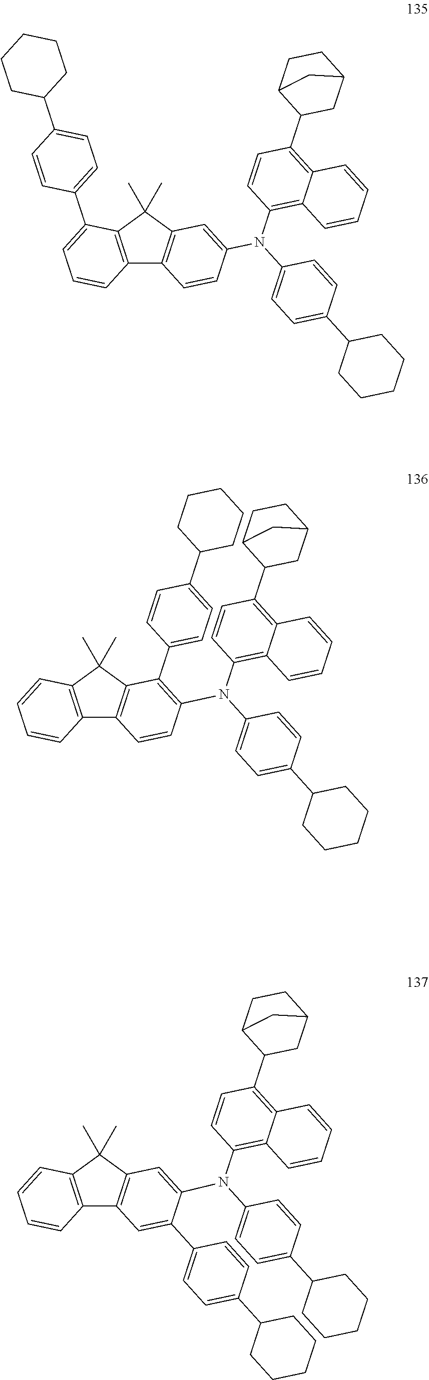 [SMILES: CC1(C)c2cc(N(c3ccc(C4CCCCC4)cc3)c3ccc(C4CC5CCC4C5)c4ccccc34)ccc2-c2cccc(-c3ccc(C4CCCCC4)cc3)c21 ; CC1(C)c2ccccc2-c2ccc(N(c3ccc(C4CCCCC4)cc3)c3ccc(C4CC5CCC4C5)c4ccccc34)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C4CC5CCC4C5)c4ccccc34)cc21]

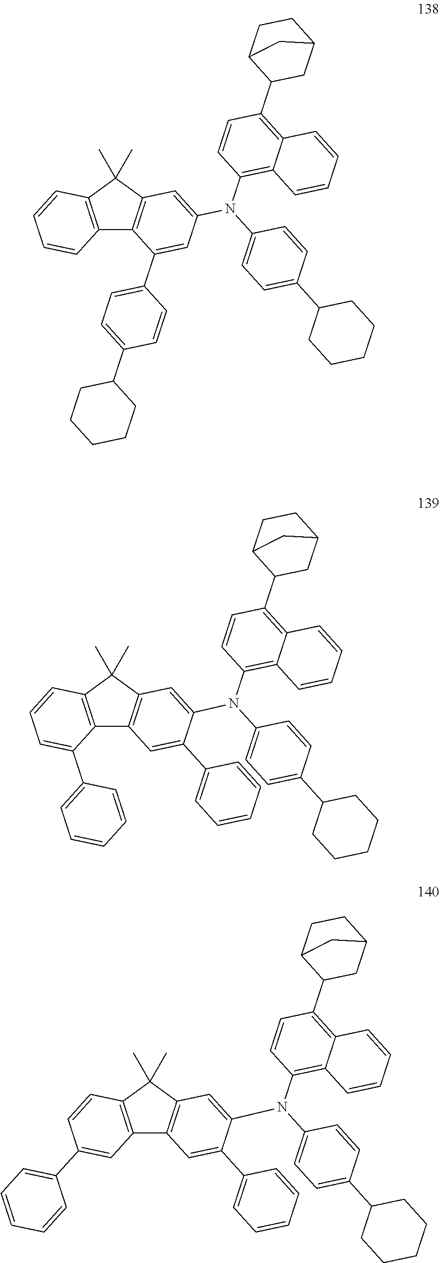 [SMILES: CC1(C)c2ccccc2-c2c(-c3ccc(C4CCCCC4)cc3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4CC5CCC4C5)c4ccccc34)cc21]

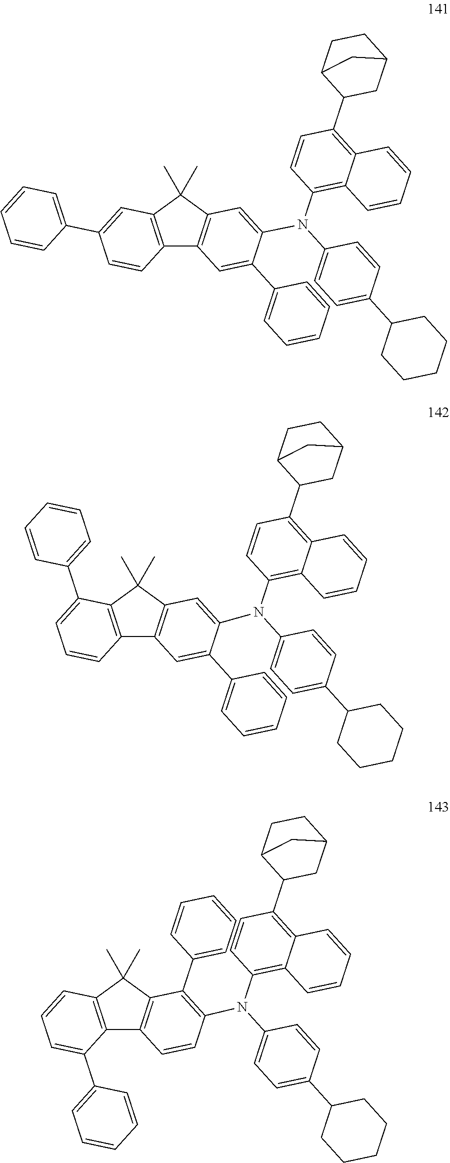 [SMILES: CC1(C)c2cc(-c3ccccc3)ccc2-c2cc(-c3ccccc3)c(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4CC5CCC4C5)c4ccccc34)c(-c3ccccc3)c21]

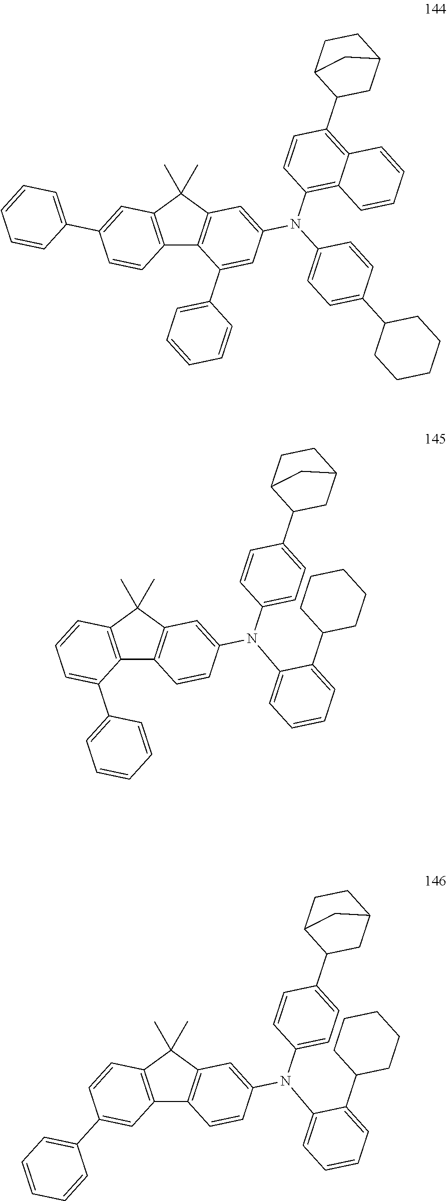 [SMILES: CC1(C)c2cc(-c3ccccc3)ccc2-c2c(-c3ccccc3)cc(N(c3ccc(C4CCCCC4)cc3)c3ccc(C4CC5CCC4C5)c4ccccc34)cc21 ; CC1(C)c2cc(N(c3ccc(C4CC5CCC4C5)cc3)c3ccccc3C3CCCCC3)ccc2-c2c(-c3ccccc3)cccc21 ; CC1(C)c2ccc(-c3ccccc3)cc2-c2ccc(N(c3ccc(C4CC5CCC4C5)cc3)c3ccccc3C3CCCCC3)cc21]

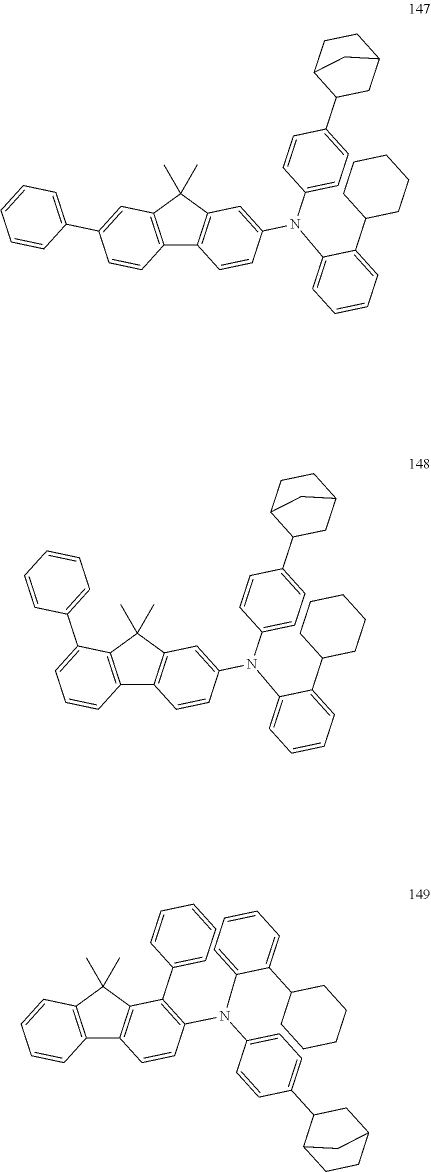 [SMILES: CC1(C)c2cc(-c3ccccc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccccc3)c21 ; CC1(C)c2ccccc2-c2ccc(N(c3ccc(C4CC5CCC4C5)cc3)c3ccccc3C3CCCCC3)c(-c3ccccc3)c21]

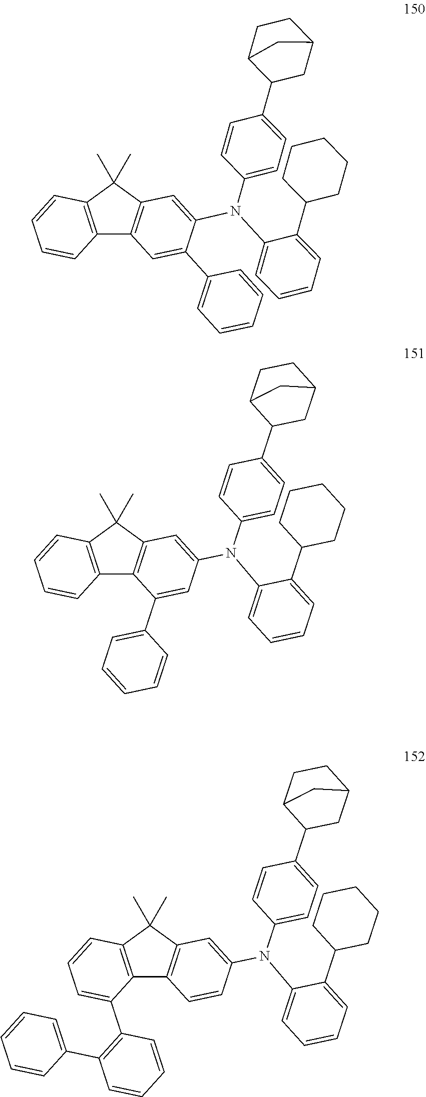

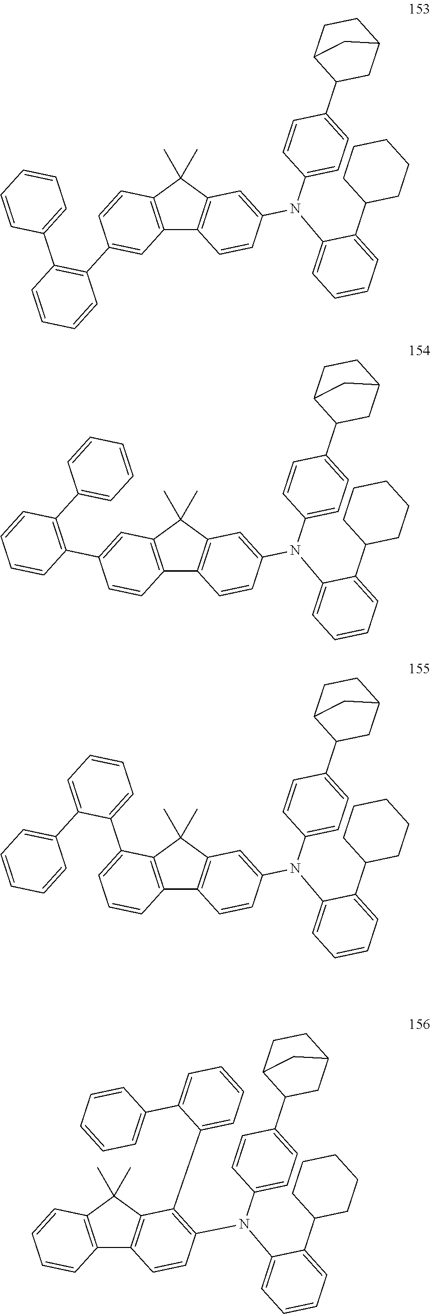

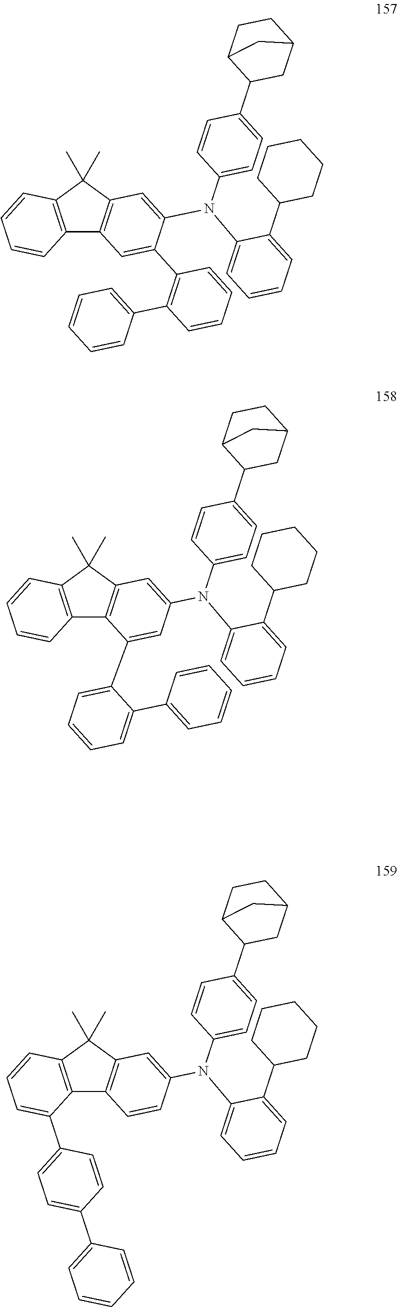 [SMILES: CC1(C)c2ccccc2-c2cc(-c3ccccc3-c3ccccc3)c(N(c3ccc(C4CC5CCC4C5)cc3)c3ccccc3C3CCCCC3)cc21 ; CC1(C)c2ccccc2-c2c(-c3ccccc3-c3ccccc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc(-c4ccccc4)cc3)cccc21]

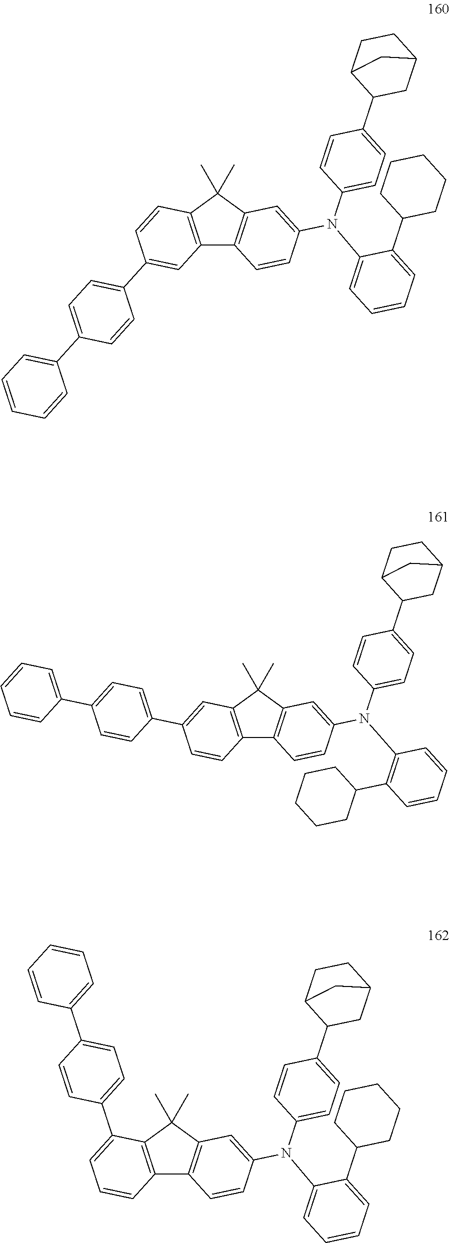 [SMILES: CC1(C)c2ccc(-c3ccc(-c4ccccc4)cc3)cc2-c2ccc(N(c3ccc(C4CC5CCC4C5)cc3)c3ccccc3C3CCCCC3)cc21 ; CC1(C)c2cc(-c3ccc(-c4ccccc4)cc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc(-c4ccccc4)cc3)c21]

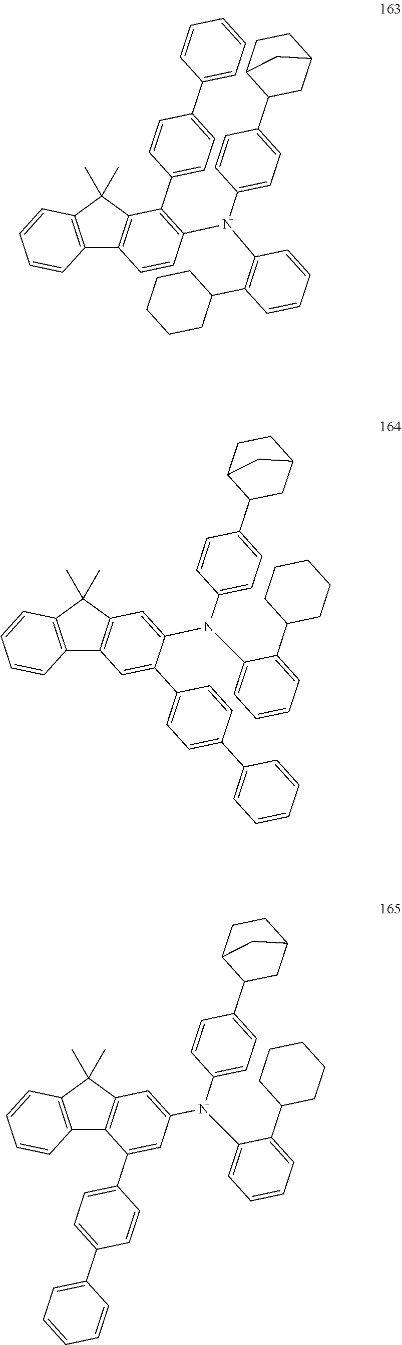 [SMILES: CC1(C)c2ccccc2-c2ccc(N(c3ccc(C4CC5CCC4C5)cc3)c3ccccc3C3CCCCC3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3ccccc3C3CCCCC3)cc21 ; CC1(C)c2ccccc2-c2c(-c3ccc(-c4ccccc4)cc3)cc(N(c3ccc(C4CC5CCC4C5)cc3)c3ccccc3C3CCCCC3)cc21]

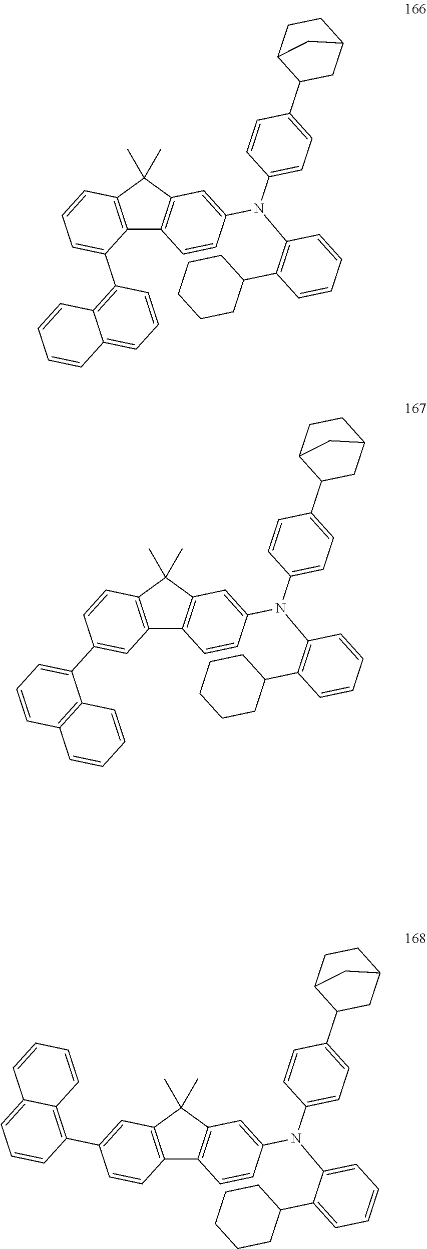 [SMILES: CC1(C)c2cc(N(c3ccc(C4CC5CCC4C5)cc3)c3ccccc3C3CCCCC3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccccc3C3CCCCC3)cc21 ; CC1(C)c2cc(-c3cccc4ccccc34)ccc2-c2ccc(N(c3ccc(C4CC5CCC4C5)cc3)c3ccccc3C3CCCCC3)cc21]

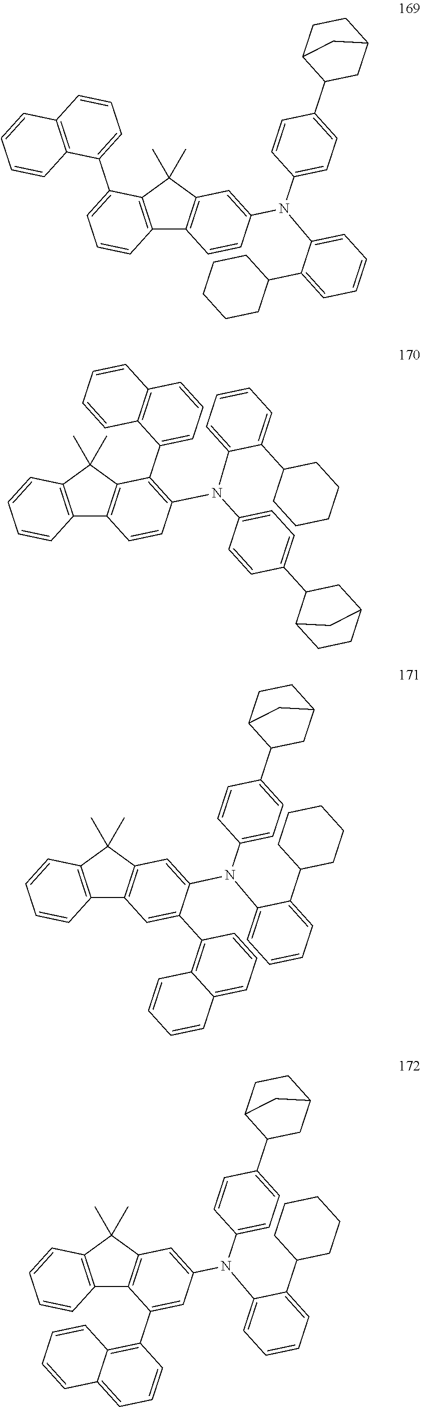

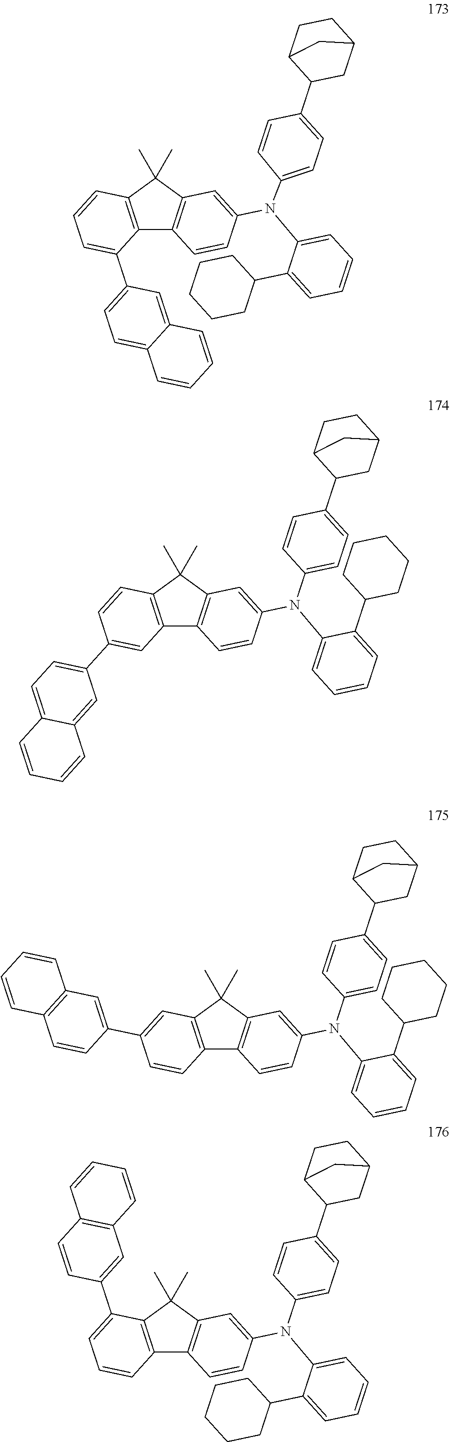

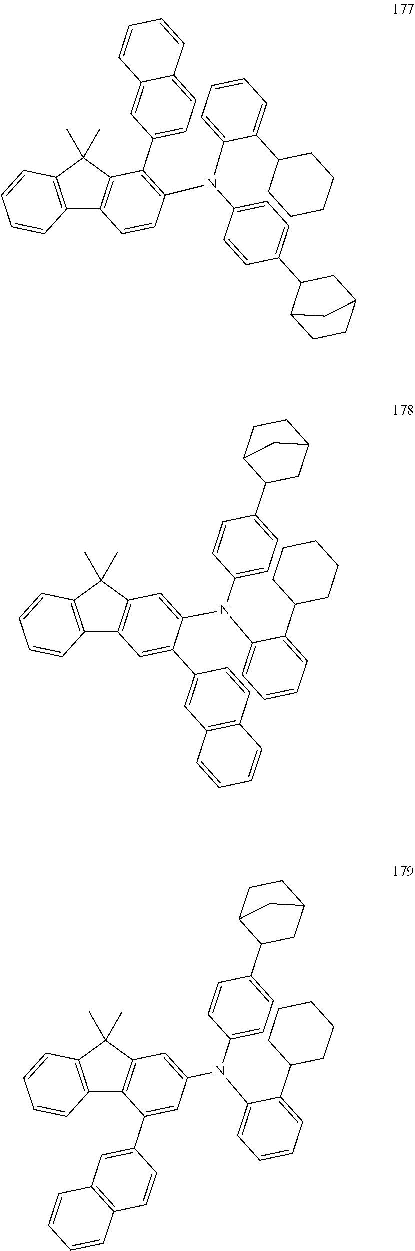 [SMILES: CC1(C)c2ccccc2-c2ccc(N(c3ccc(C4CC5CCC4C5)cc3)c3ccccc3C3CCCCC3)c(-c3ccc4ccccc4c3)c21 ; CC1(C)c2ccccc2-c2cc(-c3ccc4ccccc4c3)c(N(c3ccc(C4CC5CCC4C5)cc3)c3ccccc3C3CCCCC3)cc21 ; CC1(C)c2ccccc2-c2c(-c3ccc4ccccc4c3)cc(N(c3ccc(C4CC5CCC4C5)cc3)c3ccccc3C3CCCCC3)cc21]

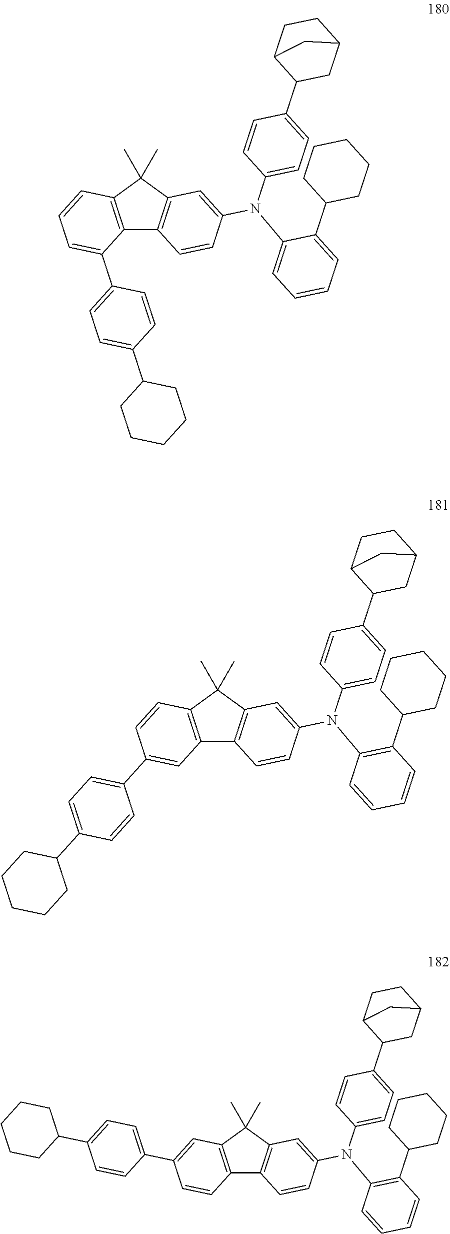 [SMILES: CC1(C)c2cc(N(c3ccc(C4CC5CCC4C5)cc3)c3ccccc3C3CCCCC3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3ccccc3C3CCCCC3)cc21 ; CC1(C)c2cc(-c3ccc(C4CCCCC4)cc3)ccc2-c2ccc(N(c3ccc(C4CC5CCC4C5)cc3)c3ccccc3C3CCCCC3)cc21]

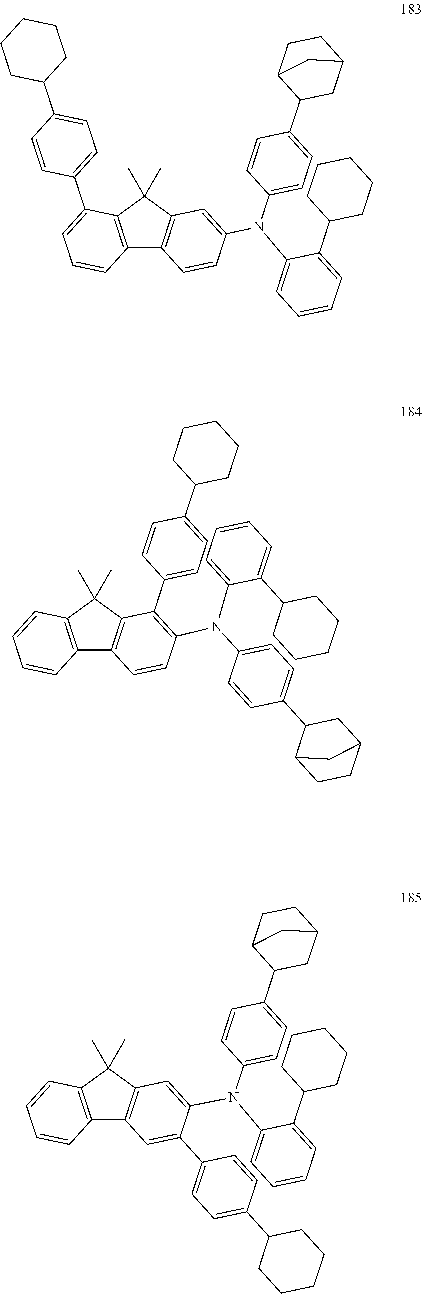 [SMILES: CC1(C)c2cc(N(c3ccc(C4CC5CCC4C5)cc3)c3ccccc3C3CCCCC3)ccc2-c2cccc(-c3ccc(C4CCCCC4)cc3)c21 ; CC1(C)c2ccccc2-c2ccc(N(c3ccc(C4CC5CCC4C5)cc3)c3ccccc3C3CCCCC3)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3ccccc3C3CCCCC3)cc21]

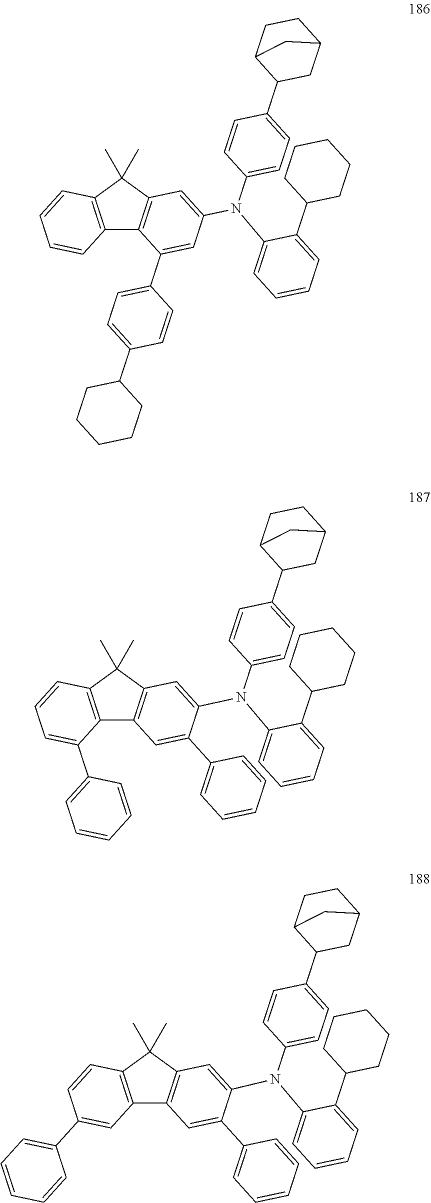 [SMILES: CC1(C)c2ccccc2-c2c(-c3ccc(C4CCCCC4)cc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)c(-c3ccccc3)cc2-c2c(-c3ccccc3)cccc21 ; CC1(C)c2ccc(-c3ccccc3)cc2-c2cc(-c3ccccc3)c(N(c3ccc(C4CC5CCC4C5)cc3)c3ccccc3C3CCCCC3)cc21]

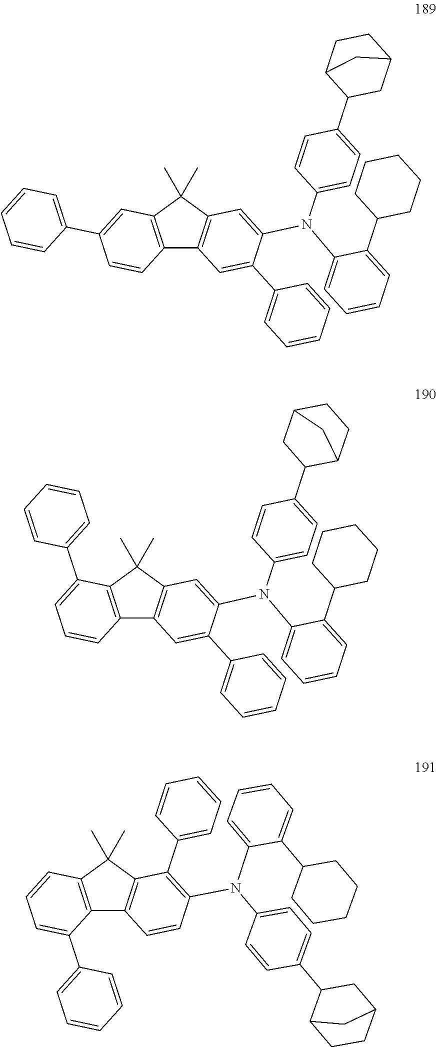 [SMILES: CC1(C)c2cc(-c3ccccc3)ccc2-c2cc(-c3ccccc3)c(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3ccccc3C3CCCCC3)c(-c3ccccc3)c21]

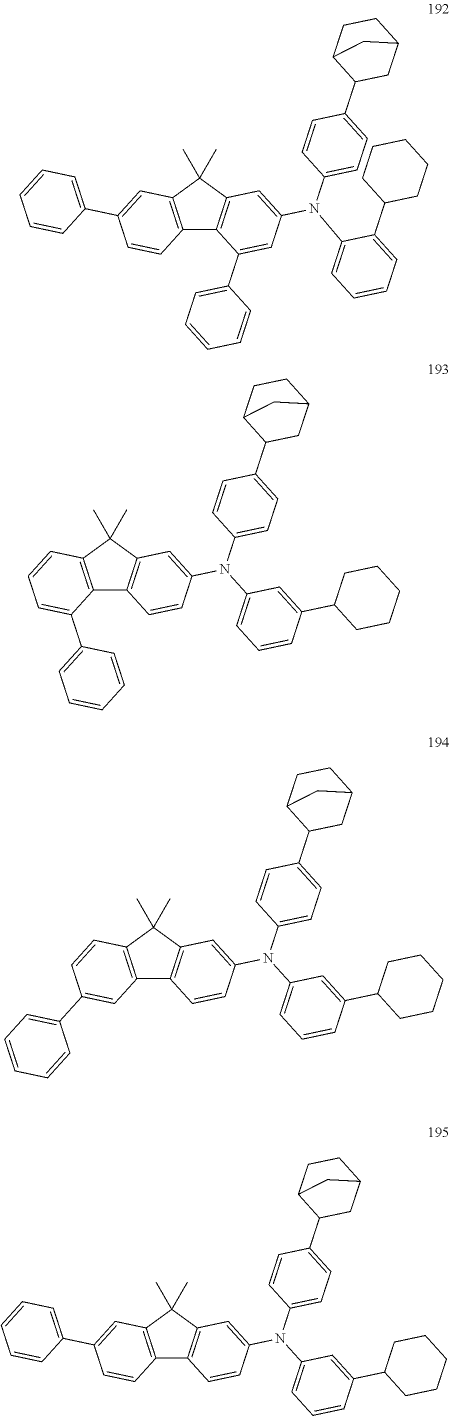

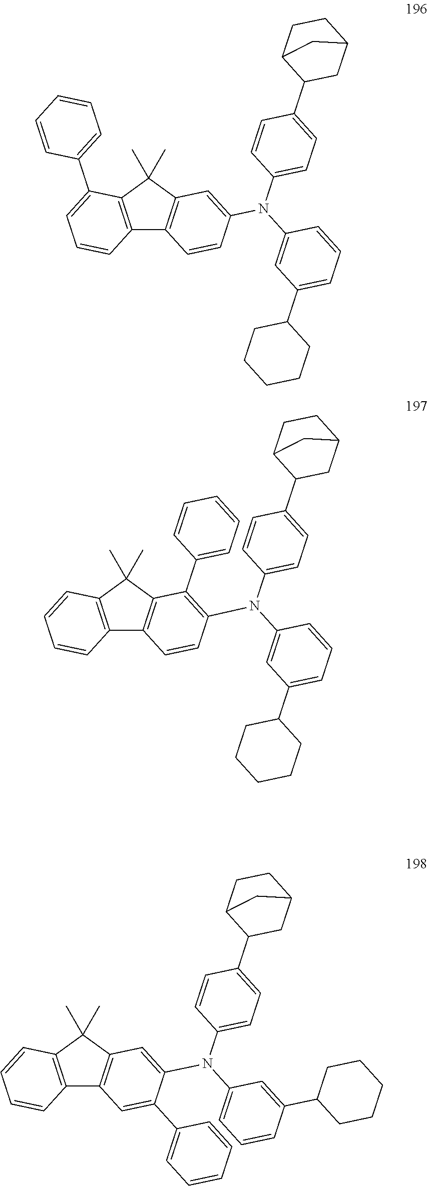

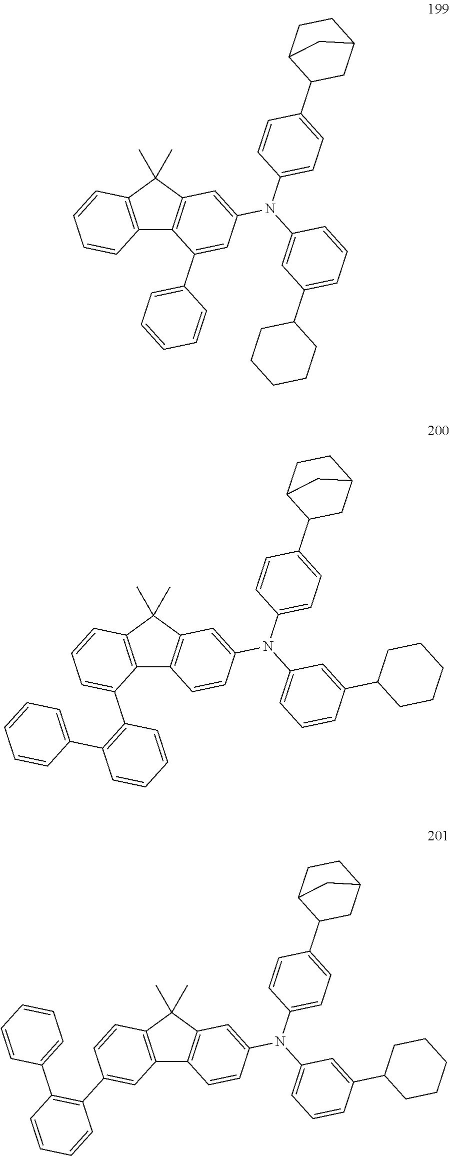 [SMILES: CC1(C)c2ccccc2-c2c(-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3cccc(C4CCCCC4)c3)cc21]

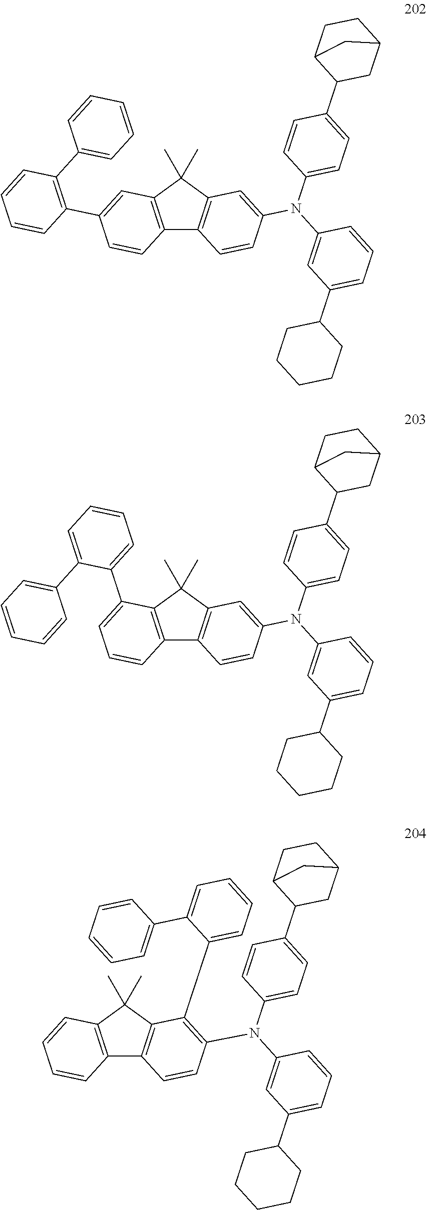 [SMILES: CC1(C)c2cc(-c3ccccc3-c3ccccc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccccc3-c3ccccc3)c21 ; CC1(C)c2ccccc2-c2ccc(N(c3ccc(C4CC5CCC4C5)cc3)c3cccc(C4CCCCC4)c3)c(-c3ccccc3-c3ccccc3)c21]

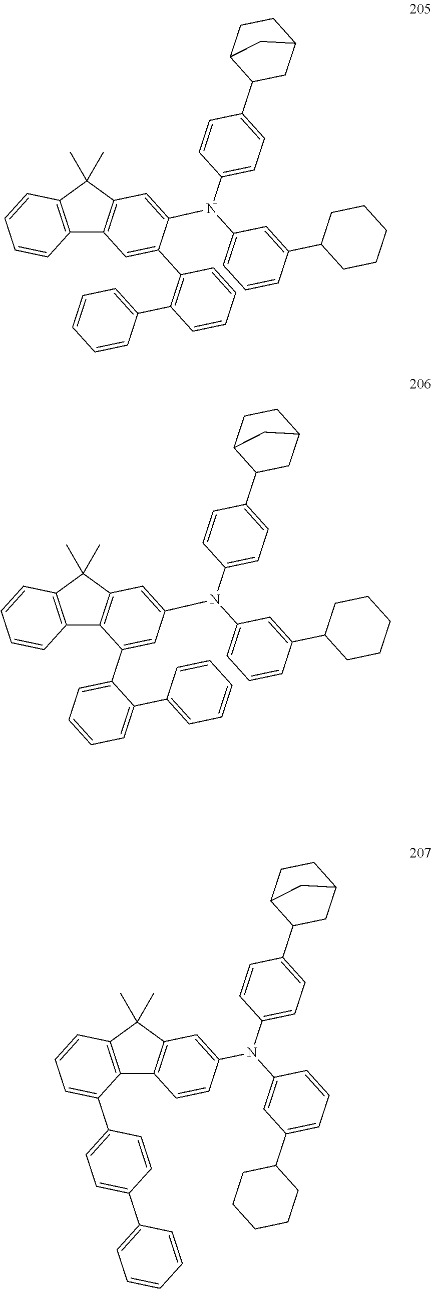 [SMILES: CC1(C)c2ccccc2-c2cc(-c3ccccc3-c3ccccc3)c(N(c3ccc(C4CC5CCC4C5)cc3)c3cccc(C4CCCCC4)c3)cc21 ; CC1(C)c2ccccc2-c2c(-c3ccccc3-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc(-c4ccccc4)cc3)cccc21]

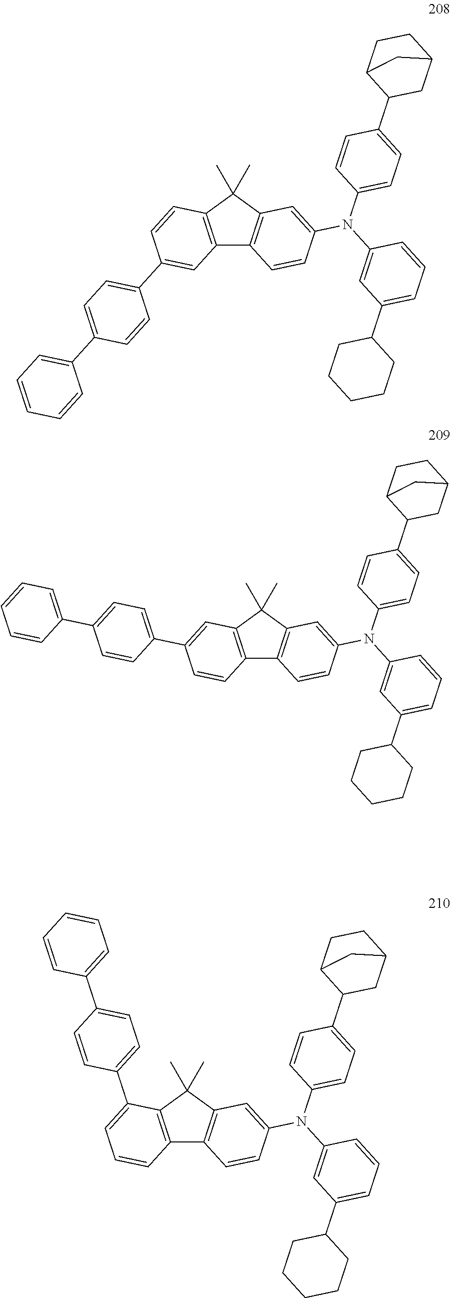 [SMILES: CC1(C)c2ccc(-c3ccc(-c4ccccc4)cc3)cc2-c2ccc(N(c3ccc(C4CC5CCC4C5)cc3)c3cccc(C4CCCCC4)c3)cc21 ; CC1(C)c2cc(-c3ccc(-c4ccccc4)cc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc(-c4ccccc4)cc3)c21]

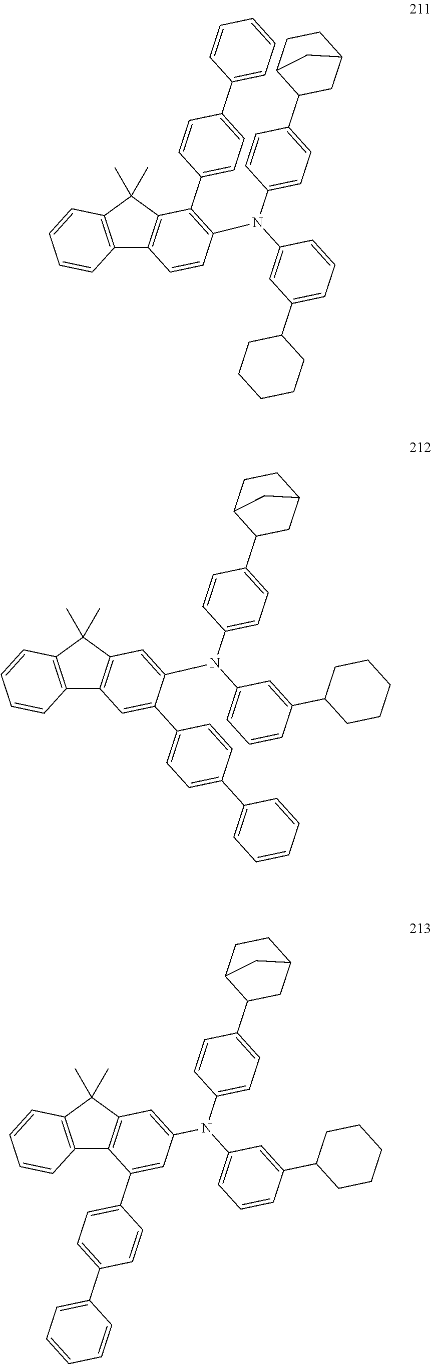 [SMILES: CC1(C)c2ccccc2-c2ccc(N(c3ccc(C4CC5CCC4C5)cc3)c3cccc(C4CCCCC4)c3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3cccc(C4CCCCC4)c3)cc21 ; CC1(C)c2ccccc2-c2c(-c3ccc(-c4ccccc4)cc3)cc(N(c3ccc(C4CC5CCC4C5)cc3)c3cccc(C4CCCCC4)c3)cc21]

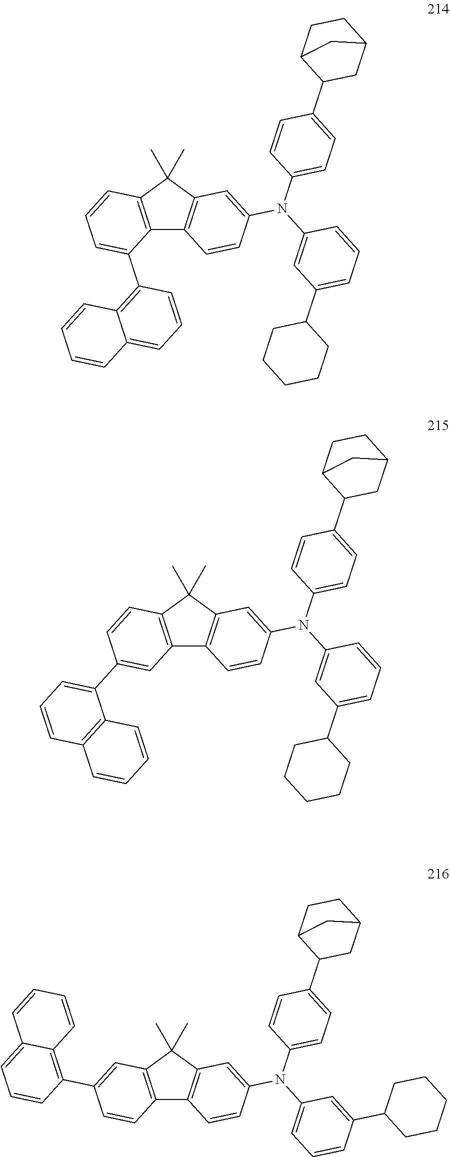 [SMILES: CC1(C)c2cc(N(c3ccc(C4CC5CCC4C5)cc3)c3cccc(C4CCCCC4)c3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cccc(C4CCCCC4)c3)cc21 ; CC1(C)c2cc(-c3cccc4ccccc34)ccc2-c2ccc(N(c3ccc(C4CC5CCC4C5)cc3)c3cccc(C4CCCCC4)c3)cc21]

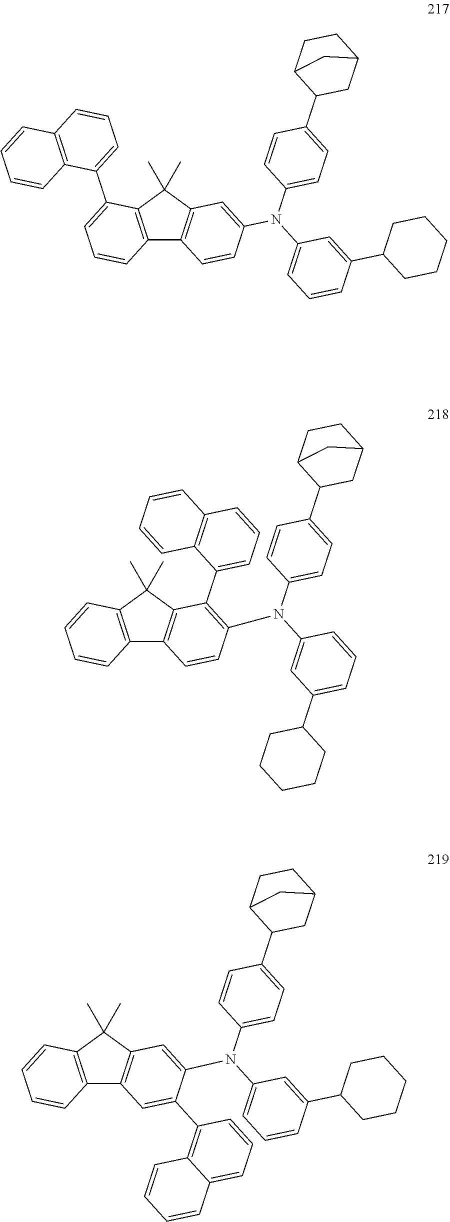 [SMILES: CC1(C)c2cc(N(c3ccc(C4CC5CCC4C5)cc3)c3cccc(C4CCCCC4)c3)ccc2-c2cccc(-c3cccc4ccccc34)c21 ; CC1(C)c2ccccc2-c2ccc(N(c3ccc(C4CC5CCC4C5)cc3)c3cccc(C4CCCCC4)c3)c(-c3cccc4ccccc34)c21 ; CC1(C)c2ccccc2-c2cc(-c3cccc4ccccc34)c(N(c3ccc(C4CC5CCC4C5)cc3)c3cccc(C4CCCCC4)c3)cc21]

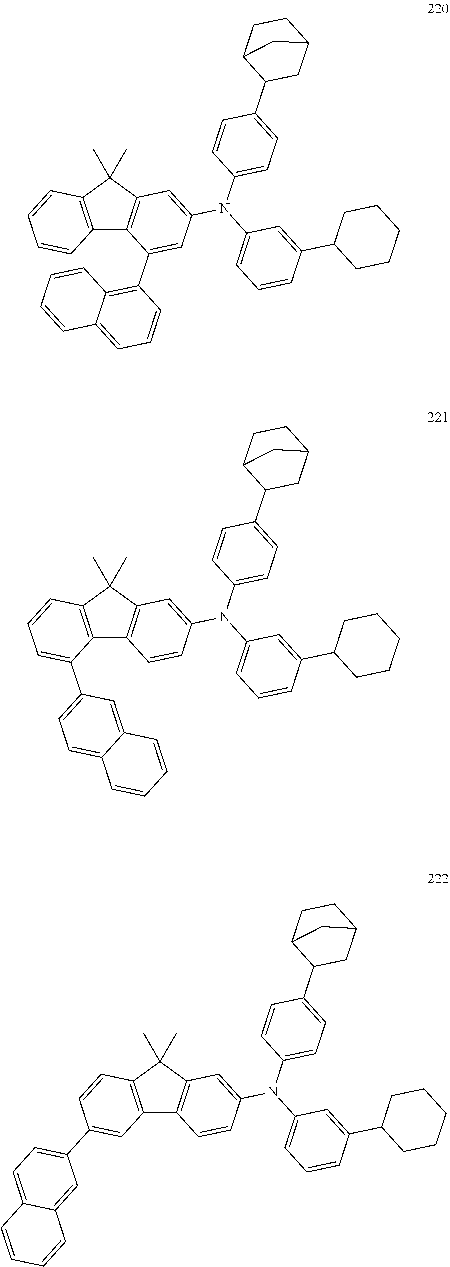 [SMILES: CC1(C)c2ccccc2-c2c(-c3cccc4ccccc34)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3cccc(C4CCCCC4)c3)cc21]

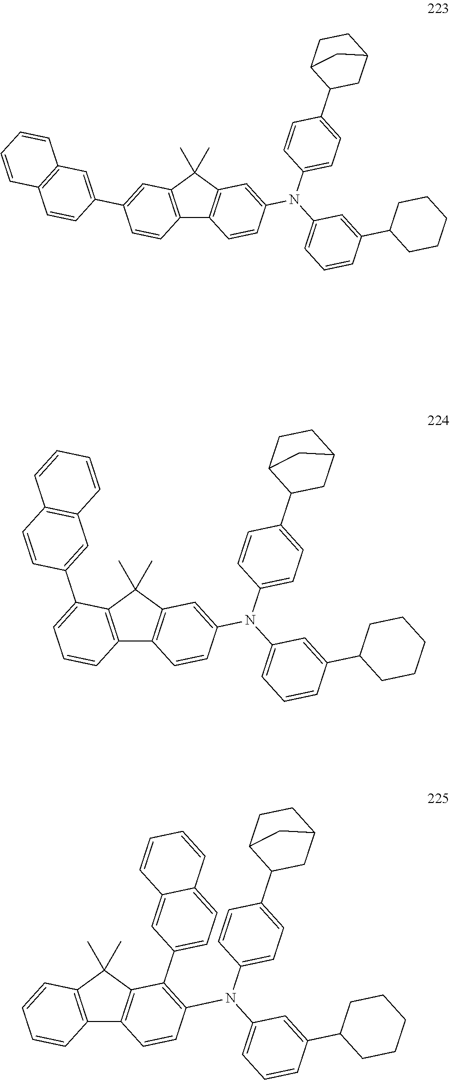 [SMILES: CC1(C)c2cc(-c3ccc4ccccc4c3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc4ccccc4c3)c21 ; CC1(C)c2ccccc2-c2ccc(N(c3ccc(C4CC5CCC4C5)cc3)c3cccc(C4CCCCC4)c3)c(-c3ccc4ccccc4c3)c21]

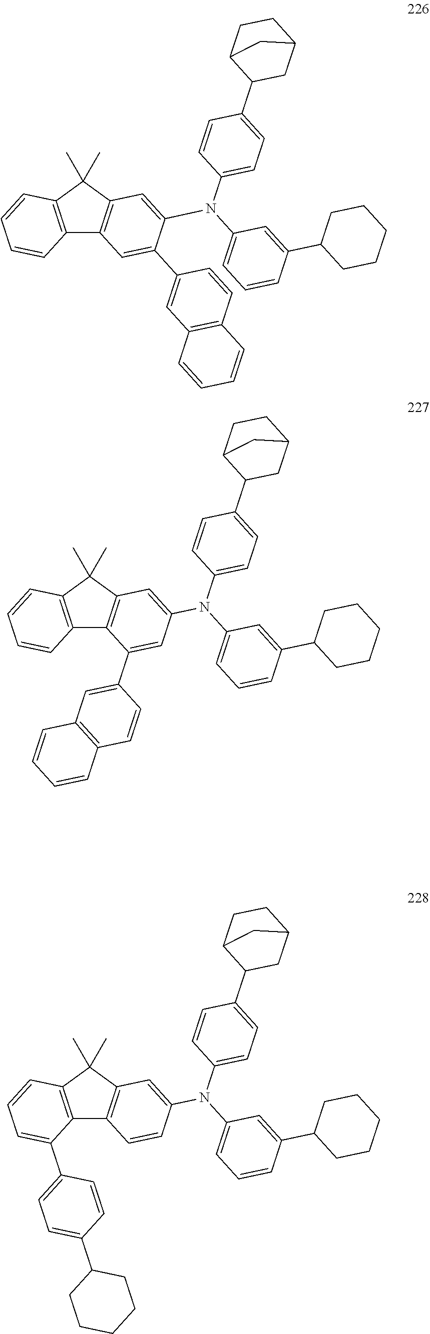

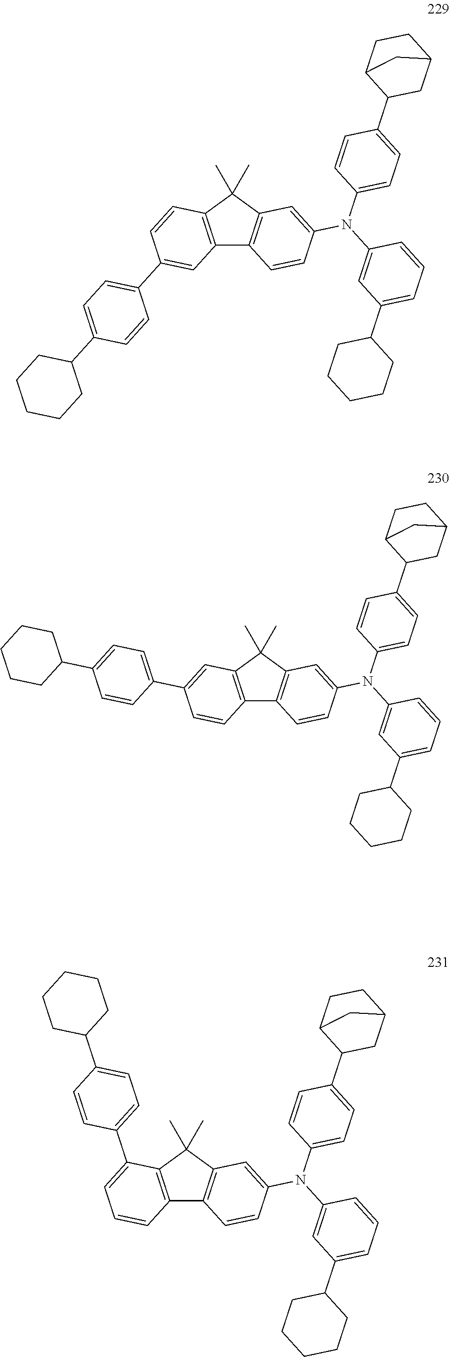 [SMILES: CC1(C)c2ccc(-c3ccc(C4CCCCC4)cc3)cc2-c2ccc(N(c3ccc(C4CC5CCC4C5)cc3)c3cccc(C4CCCCC4)c3)cc21 ; CC1(C)c2cc(-c3ccc(C4CCCCC4)cc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc(C4CCCCC4)cc3)c21]

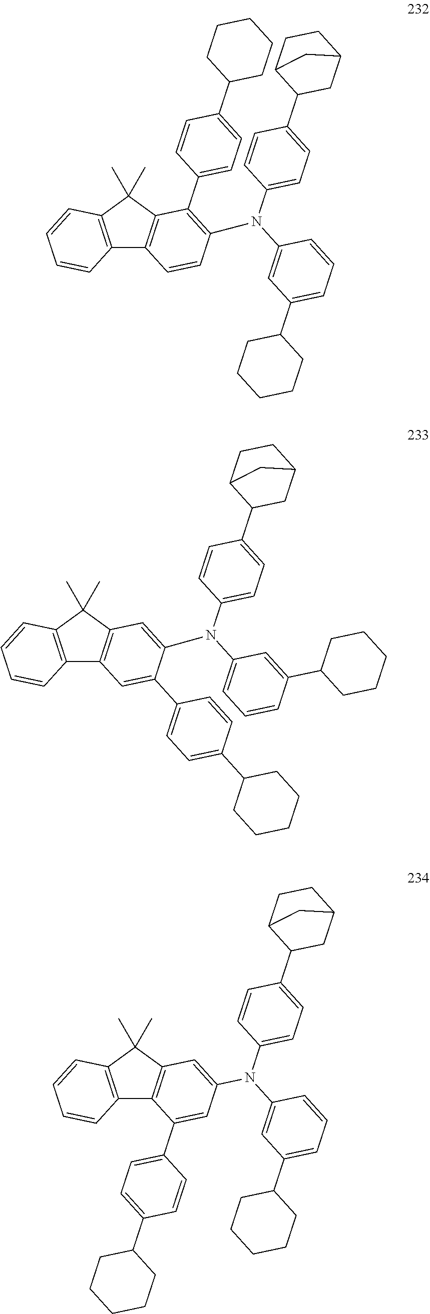 [SMILES: CC1(C)c2ccccc2-c2ccc(N(c3ccc(C4CC5CCC4C5)cc3)c3cccc(C4CCCCC4)c3)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3cccc(C4CCCCC4)c3)cc21 ; CC1(C)c2ccccc2-c2c(-c3ccc(C4CCCCC4)cc3)cc(N(c3ccc(C4CC5CCC4C5)cc3)c3cccc(C4CCCCC4)c3)cc21]

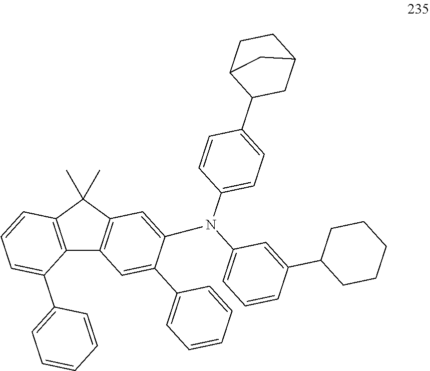 [SMILES: CC1(C)c2cc(N(c3ccc(C4CC5CCC4C5)cc3)c3cccc(C4CCCCC4)c3)c(-c3ccccc3)cc2-c2c(-c3ccccc3)cccc21]

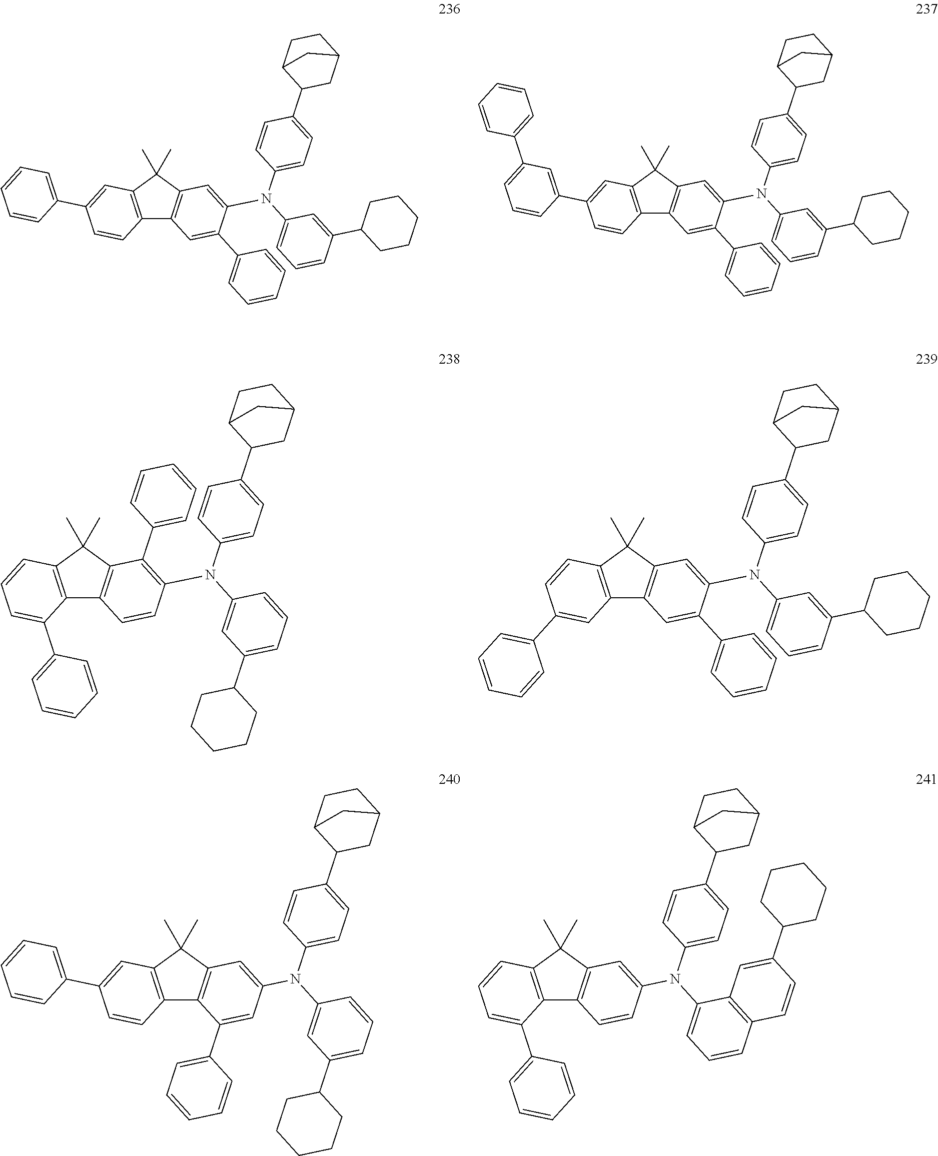

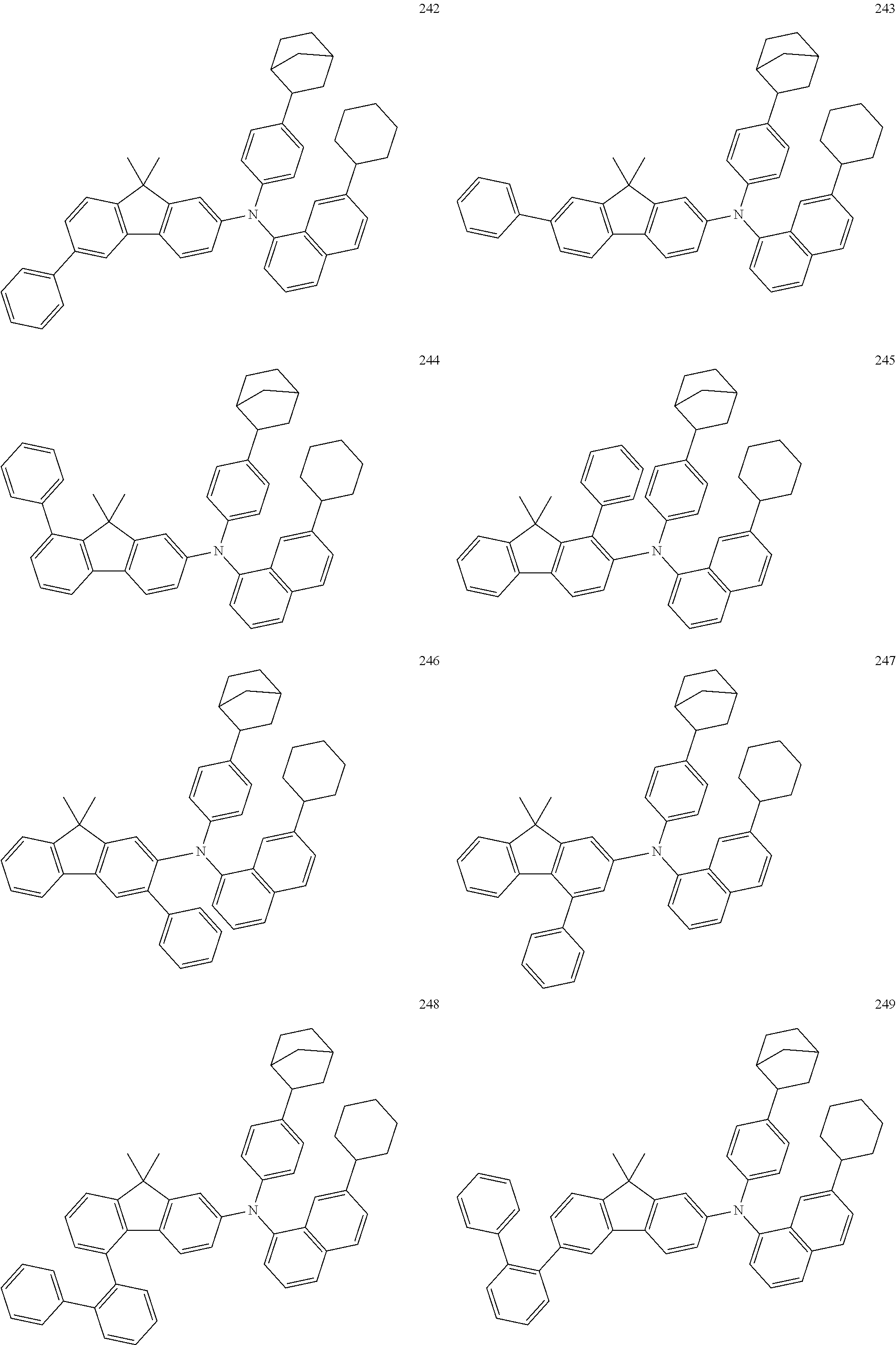

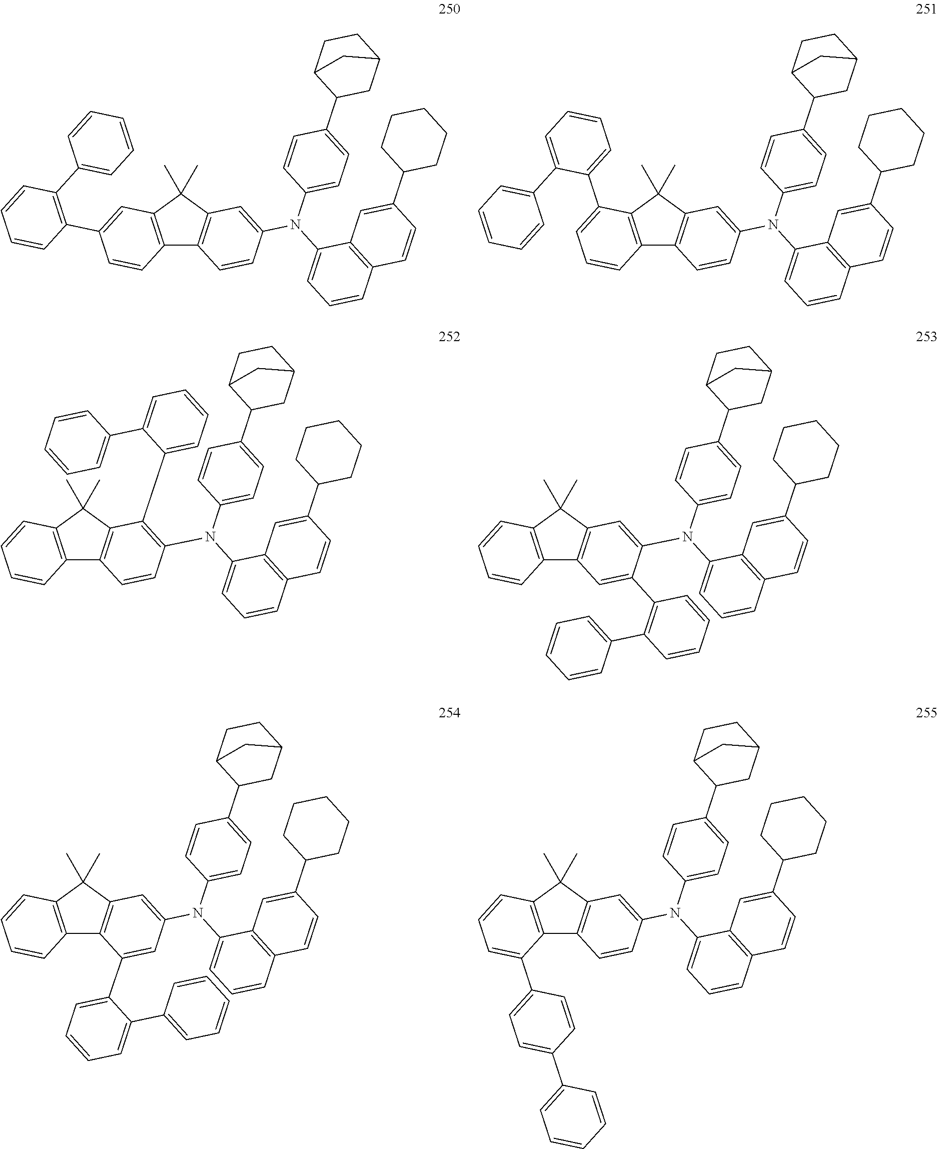

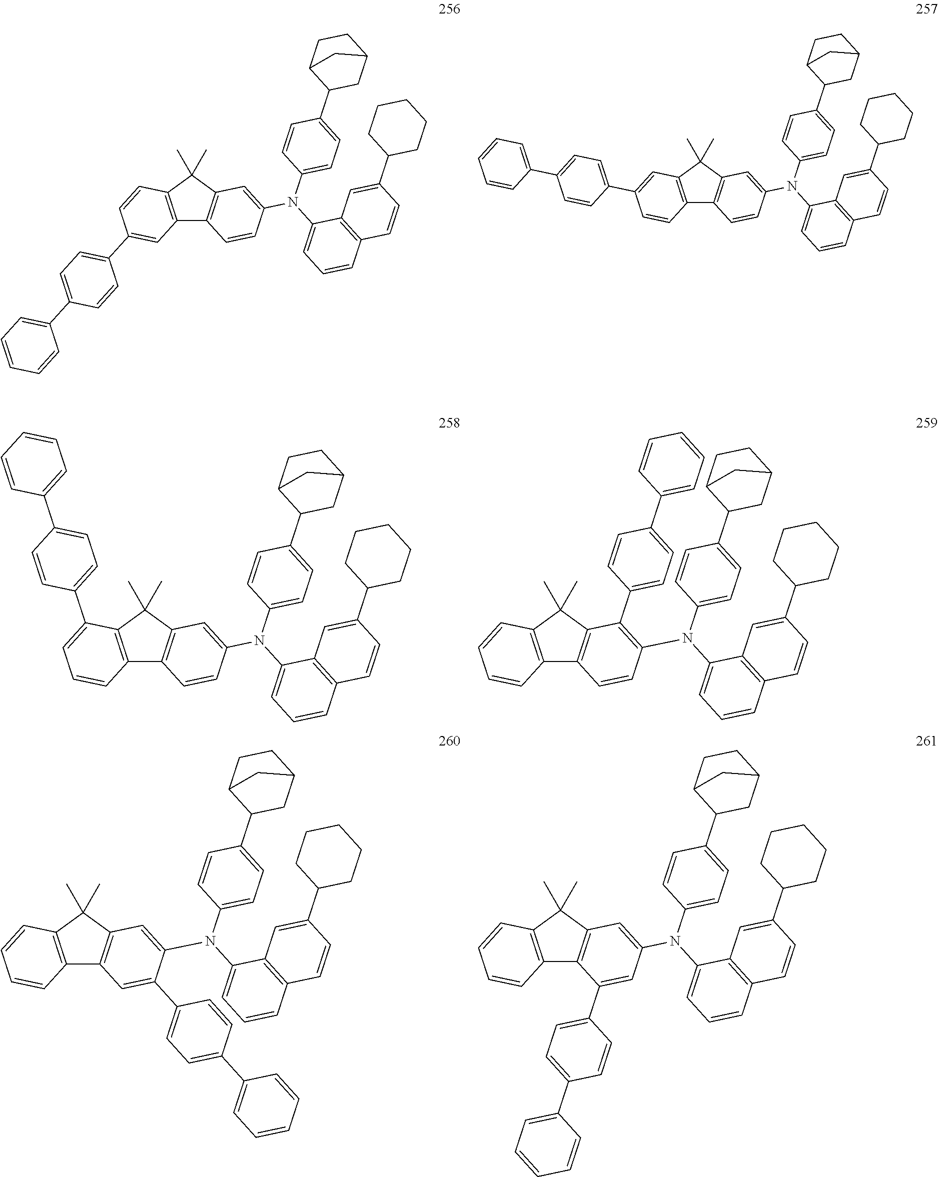

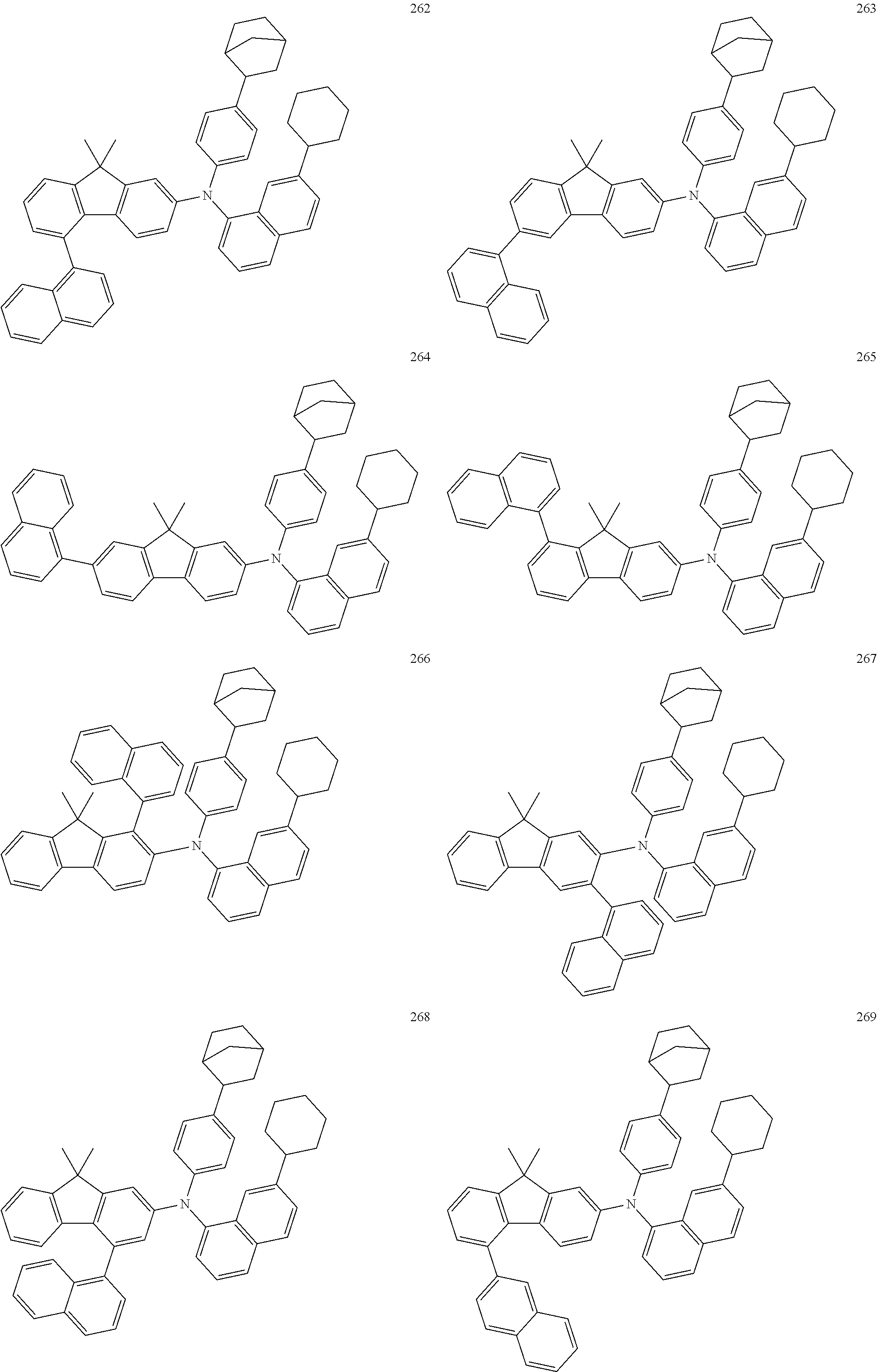

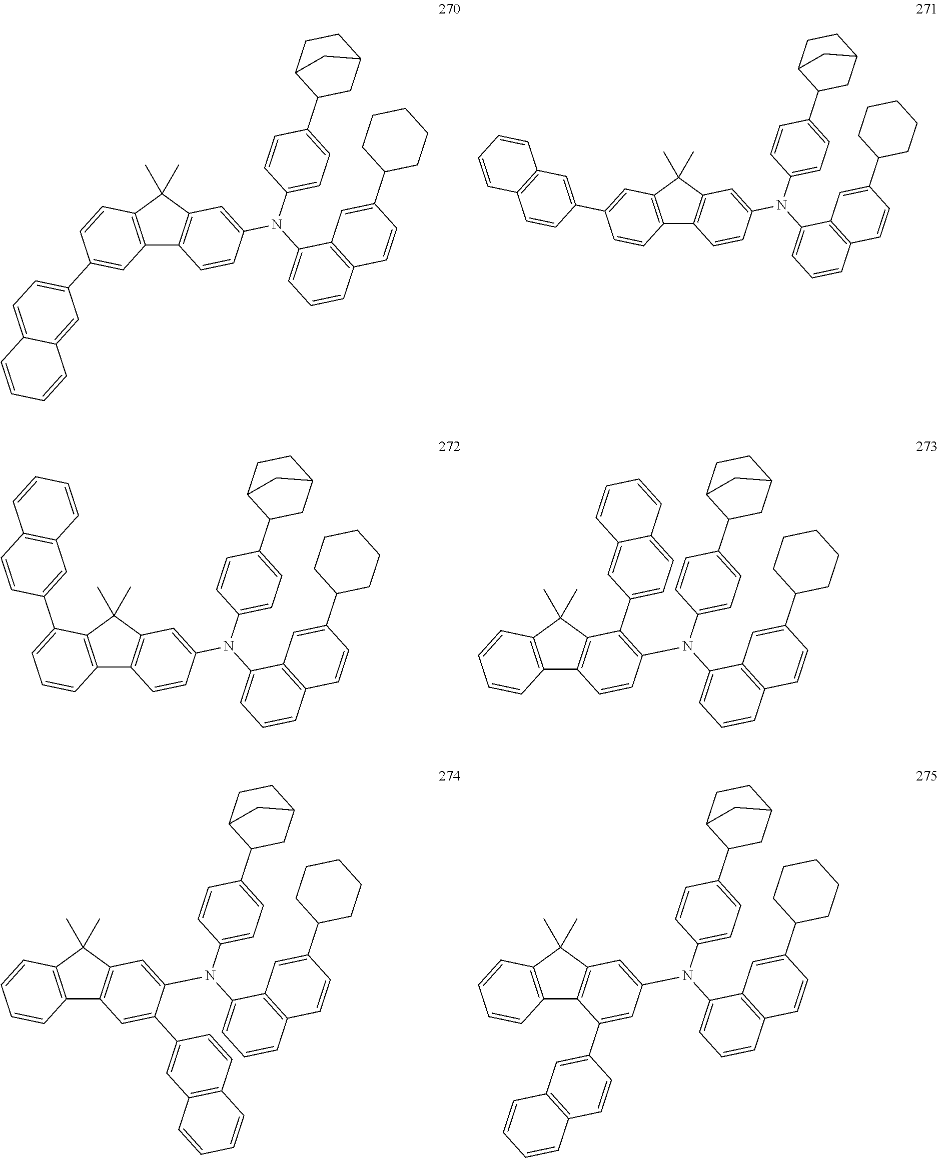

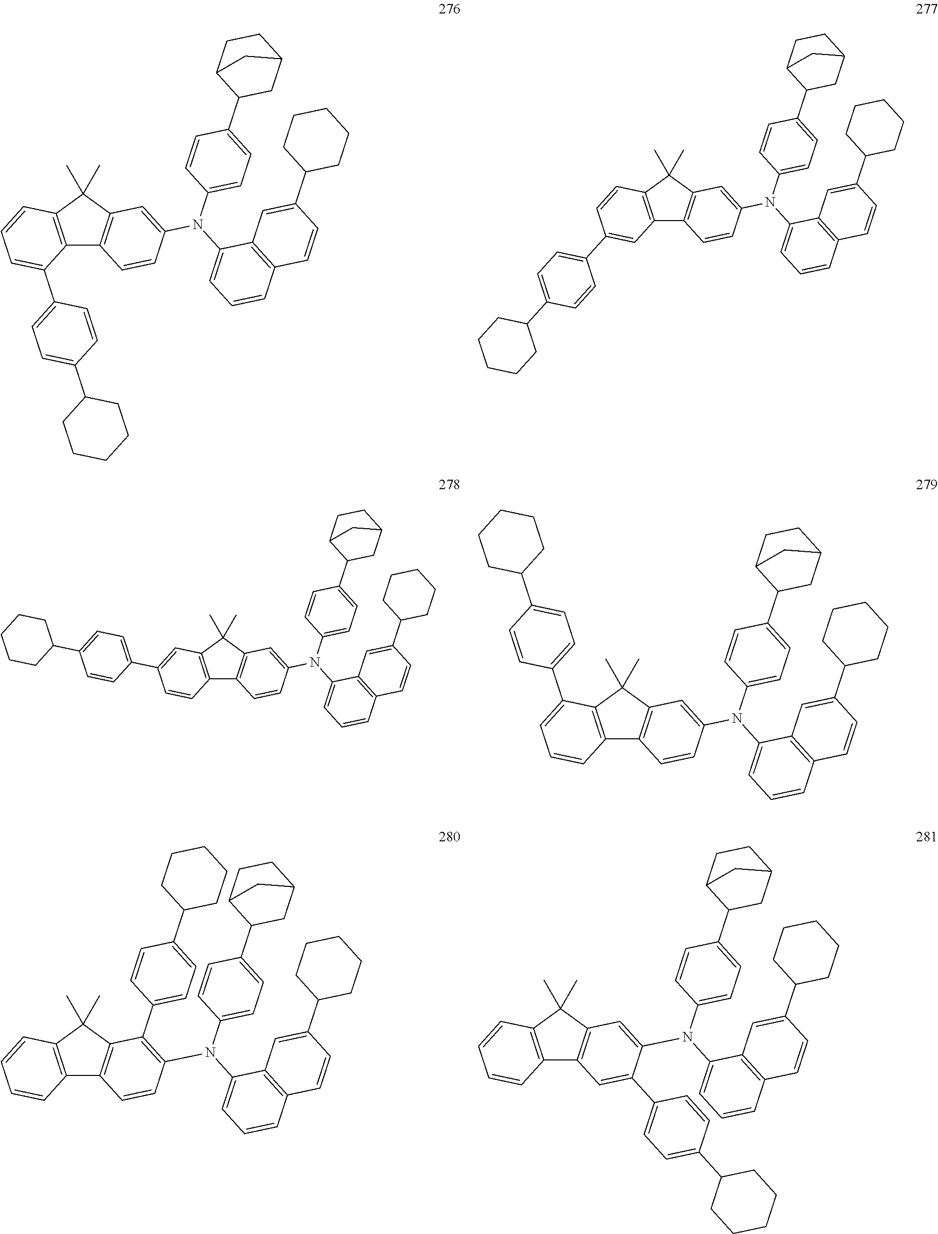

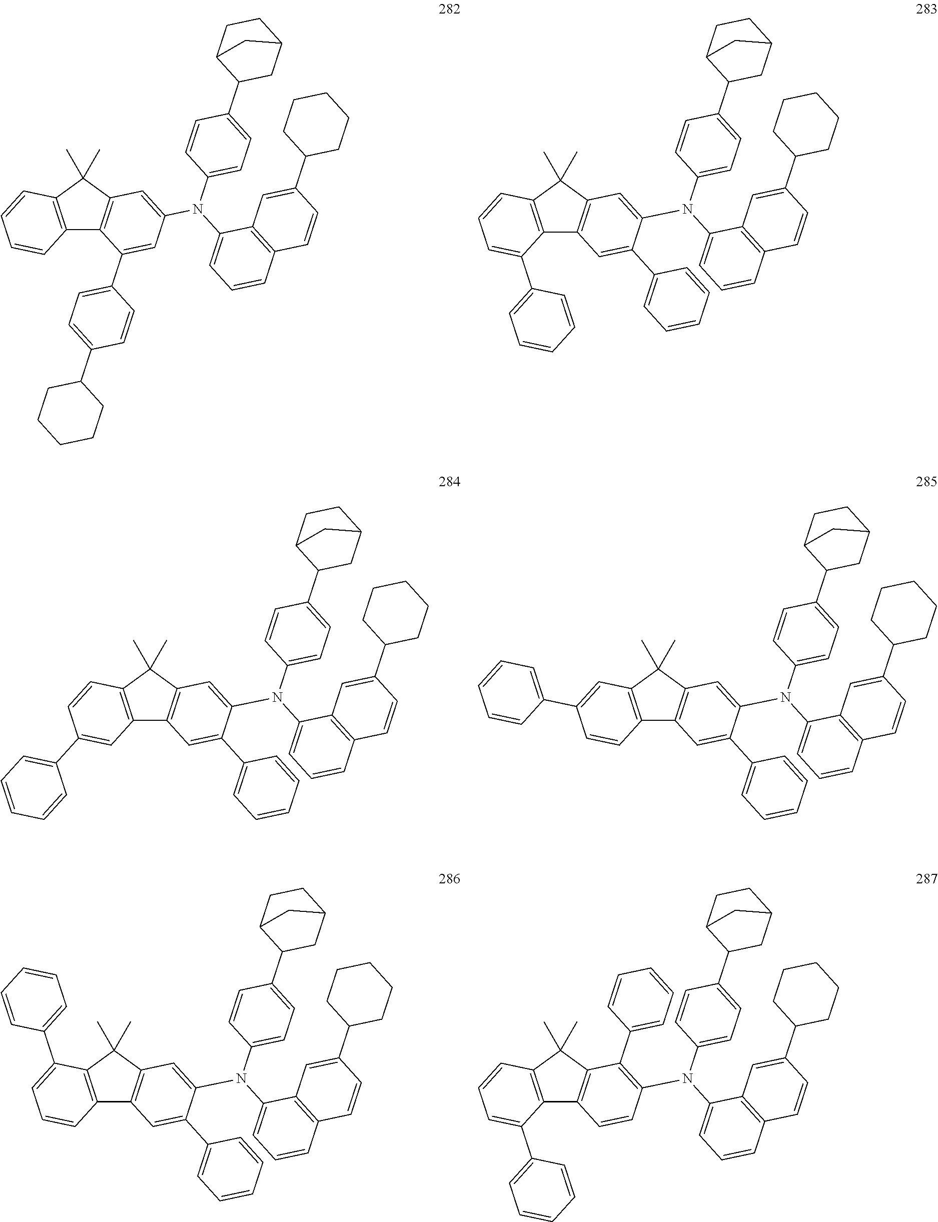

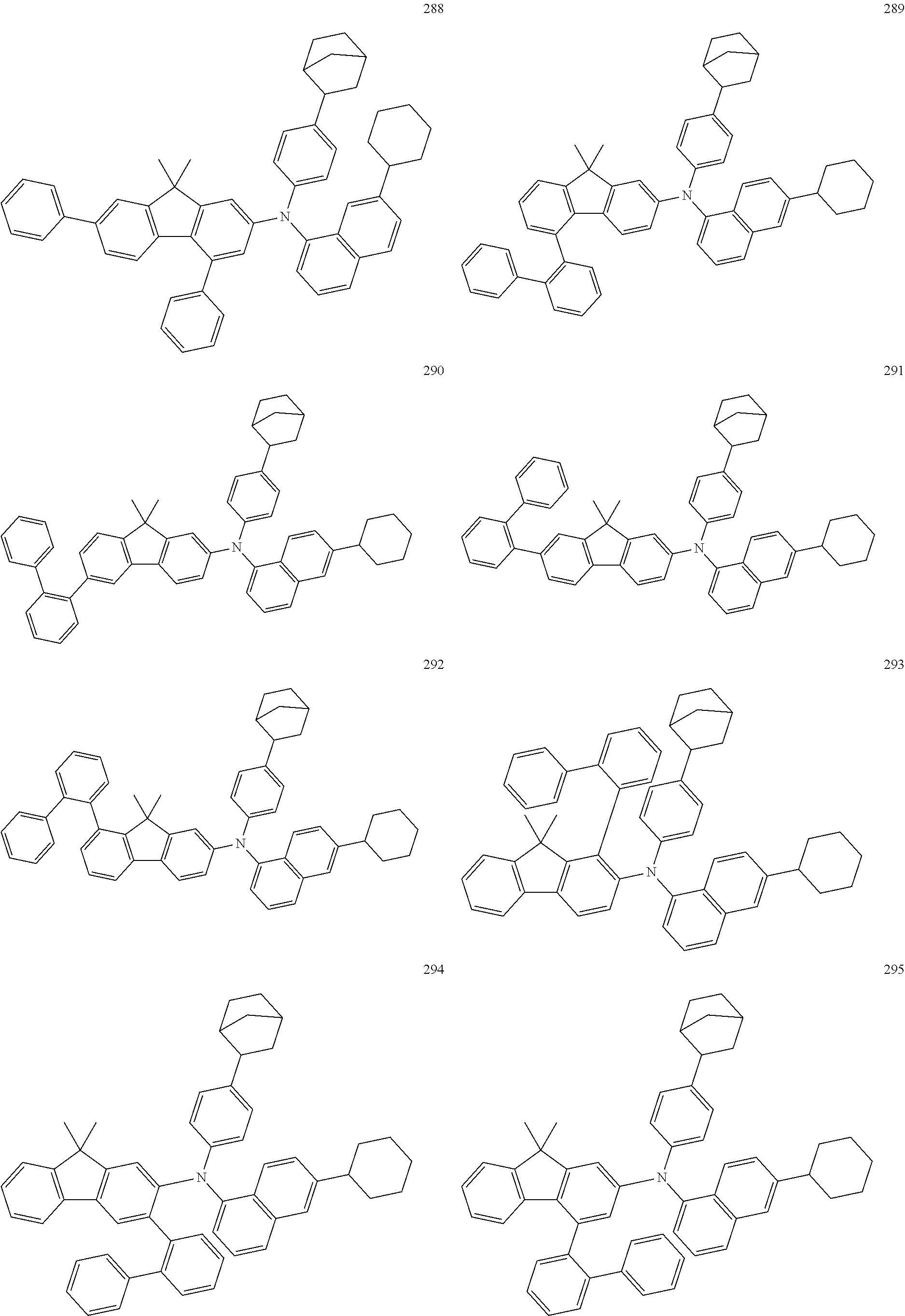

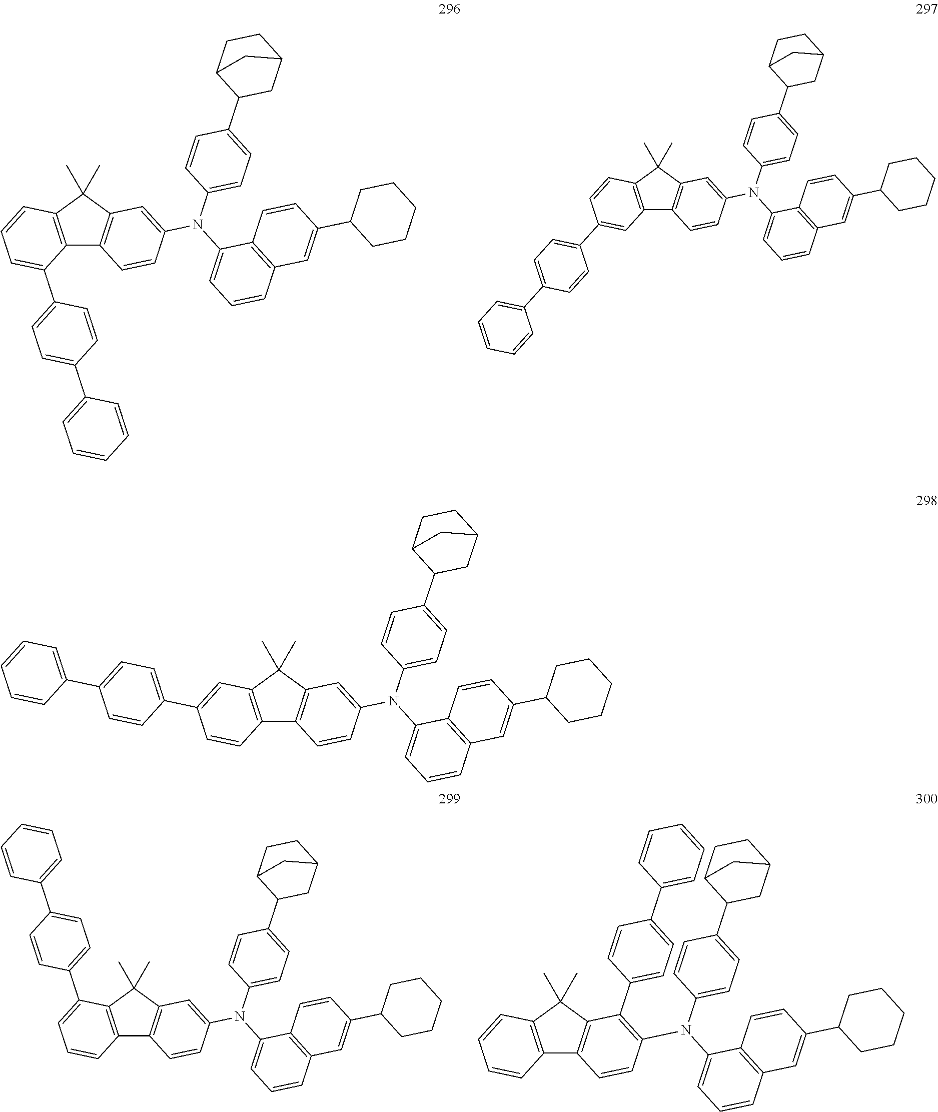 [SMILES: CC1(C)c2cc(N(c3ccc(C4CC5CCC4C5)cc3)c3cccc4cc(C5CCCCC5)ccc34)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3cccc4cc(C5CCCCC5)ccc34)cc21 ; CC1(C)c2cc(-c3ccc(-c4ccccc4)cc3)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3ccc(-c4ccccc4)cc3)c21 ; CC1(C)c2ccccc2-c2ccc(N(c3ccc(C4CC5CCC4C5)cc3)c3cccc4cc(C5CCCCC5)ccc34)c(-c3ccc(-c4ccccc4)cc3)c21]

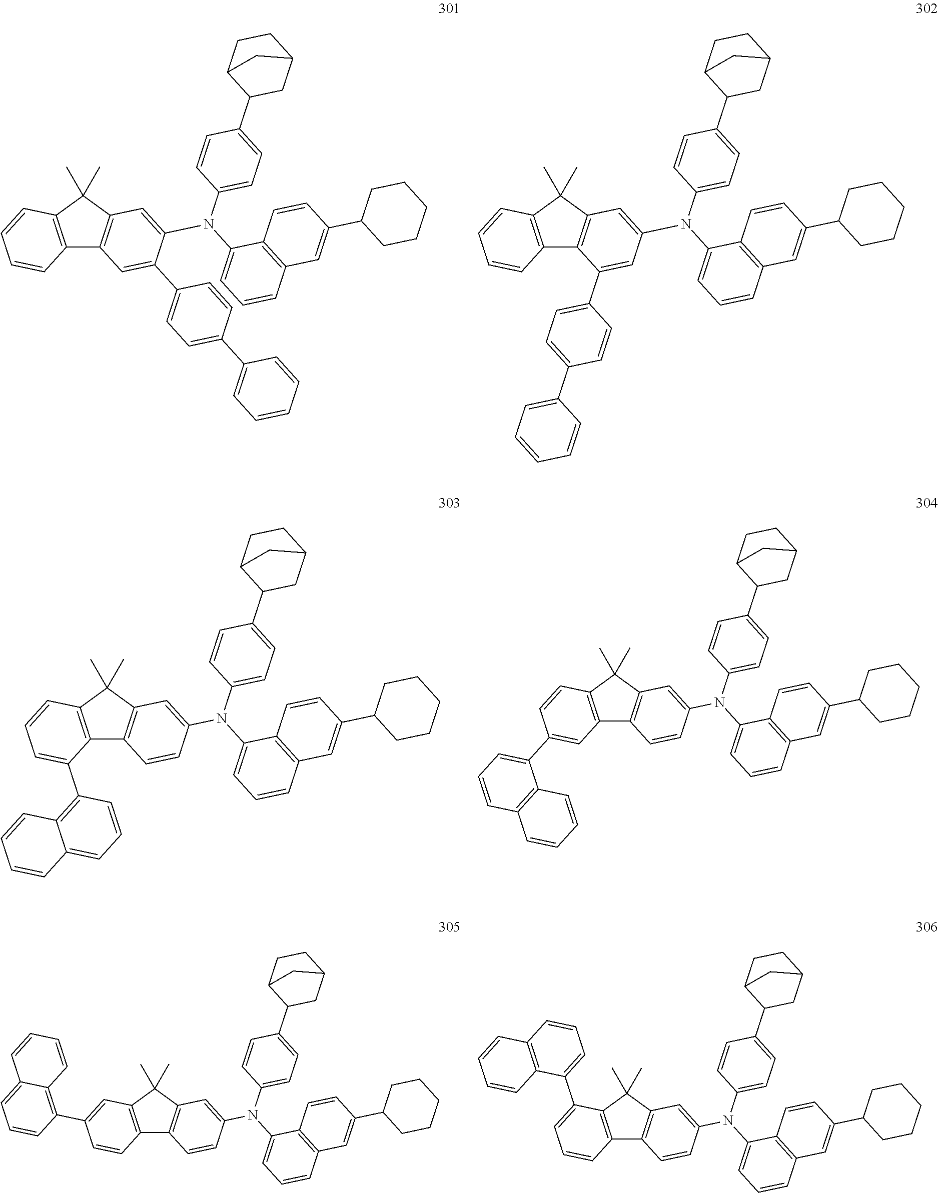

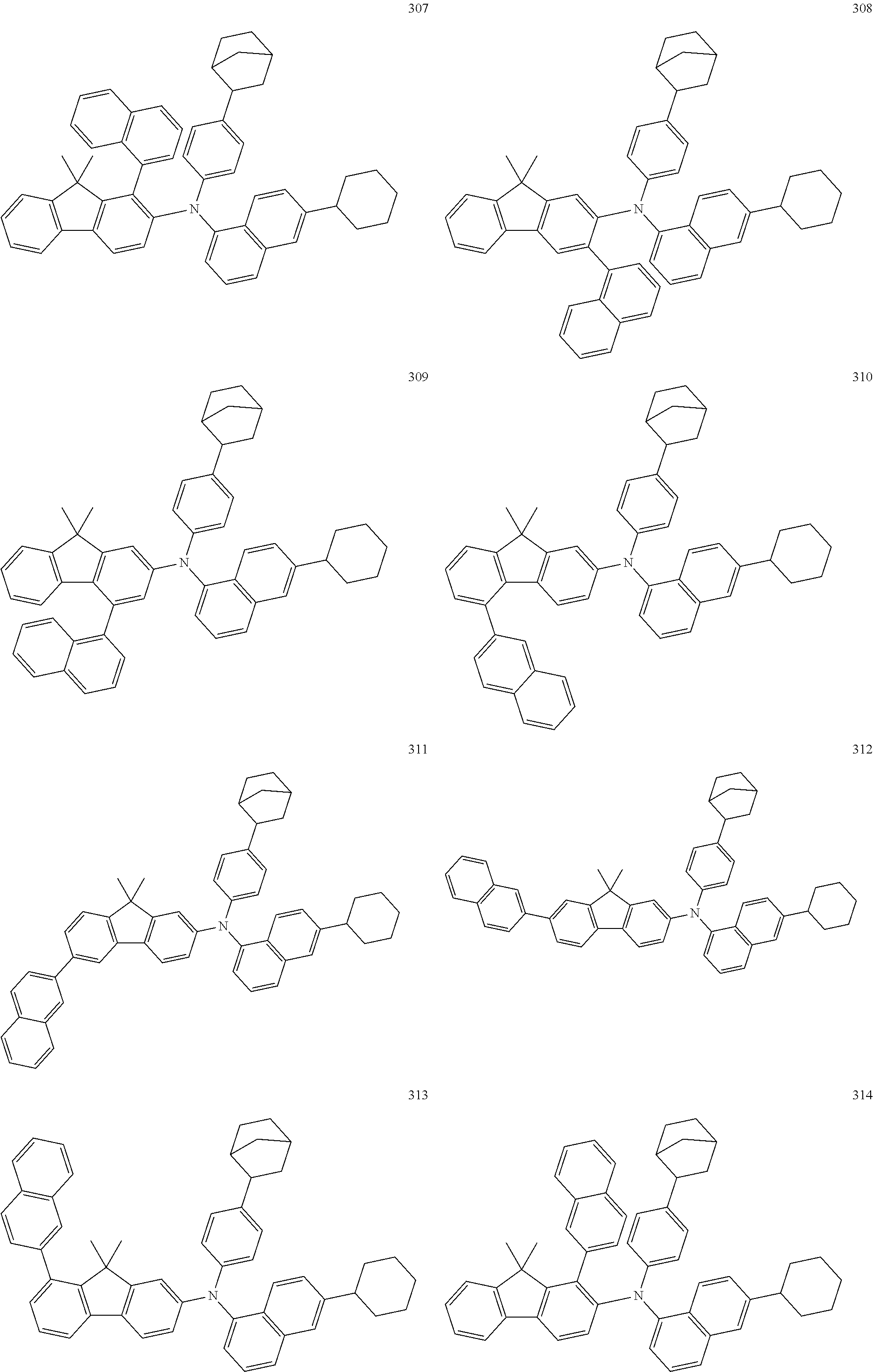

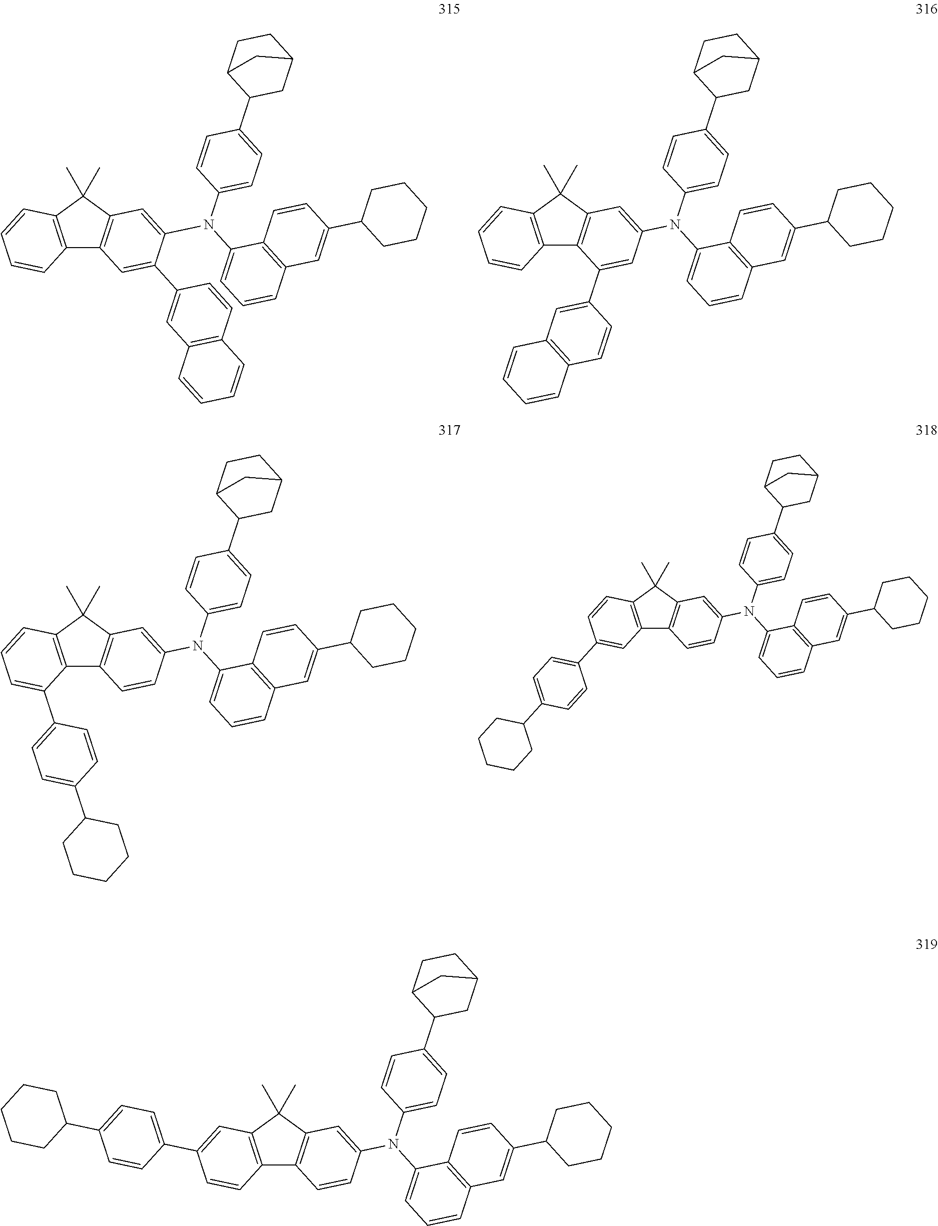 [SMILES: CC1(C)c2ccccc2-c2cc(-c3ccc4ccccc4c3)c(N(c3ccc(C4CC5CCC4C5)cc3)c3cccc4cc(C5CCCCC5)ccc34)cc21 ; CC1(C)c2ccccc2-c2c(-c3ccc4ccccc4c3)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CC5CCC4C5)cc3)c3cccc4cc(C5CCCCC5)ccc34)cc21 ; CC1(C)c2cc(-c3ccc(C4CCCCC4)cc3)ccc2-c2ccc(N(c3ccc(C4CC5CCC4C5)cc3)c3cccc4cc(C5CCCCC5)ccc34)cc21]

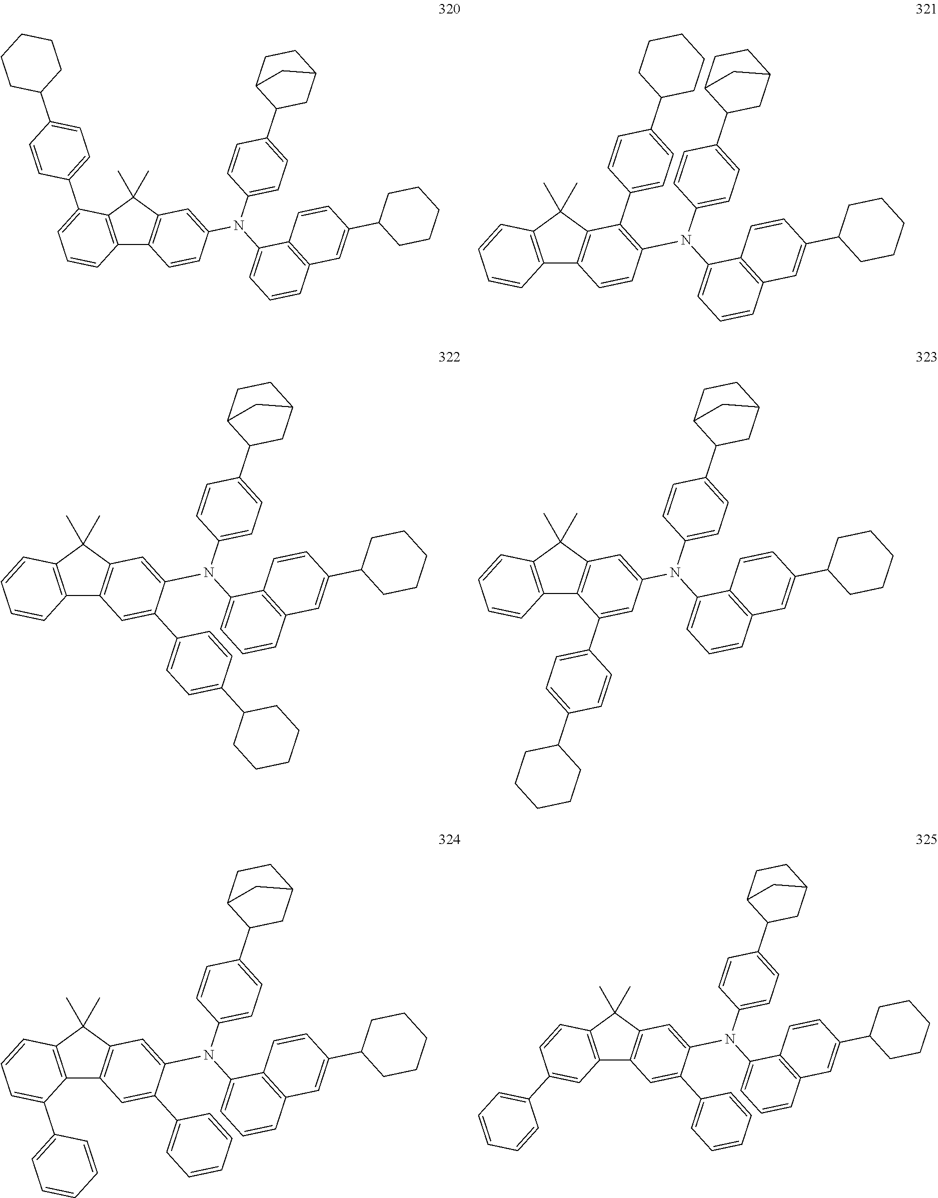

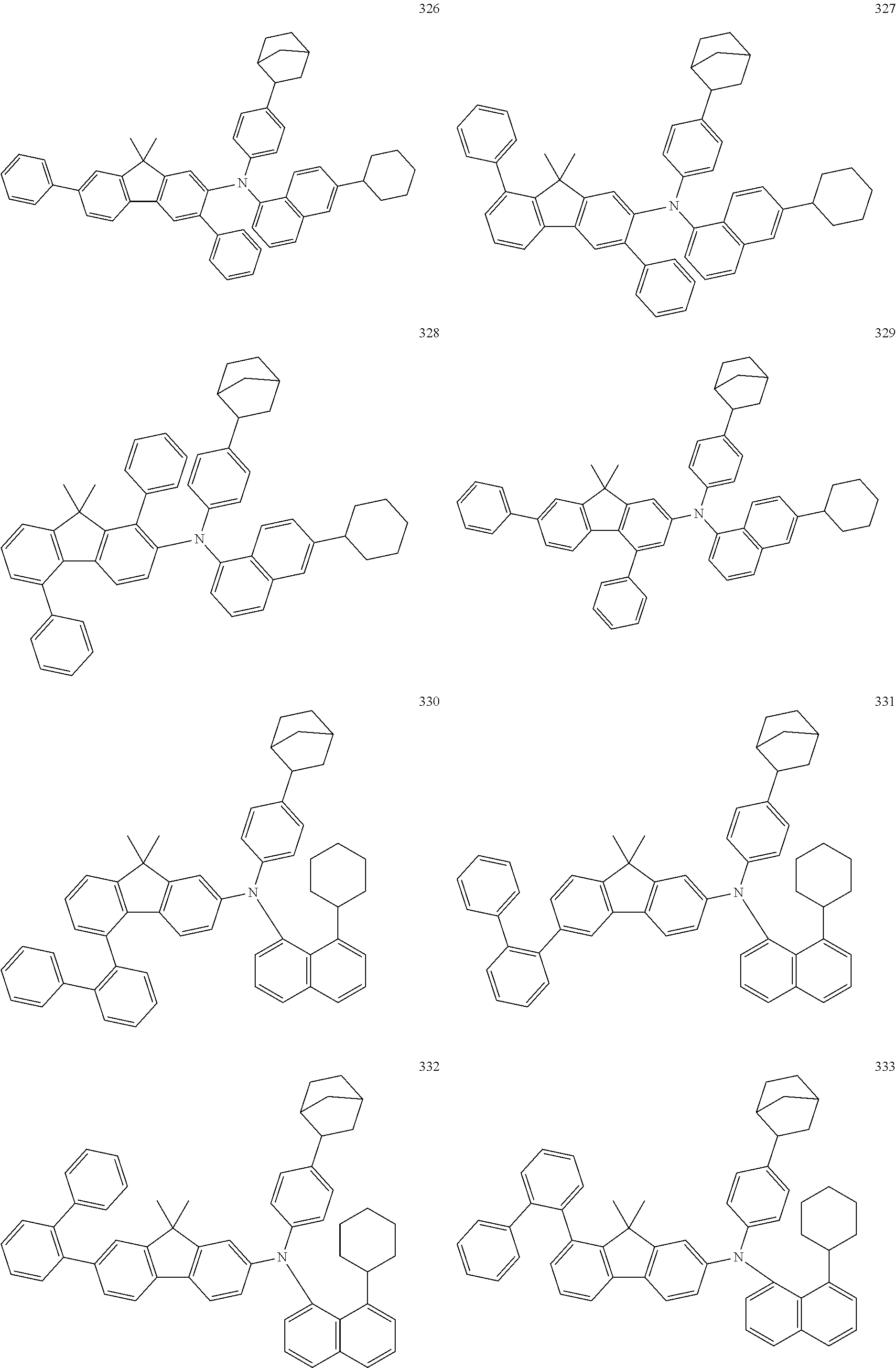

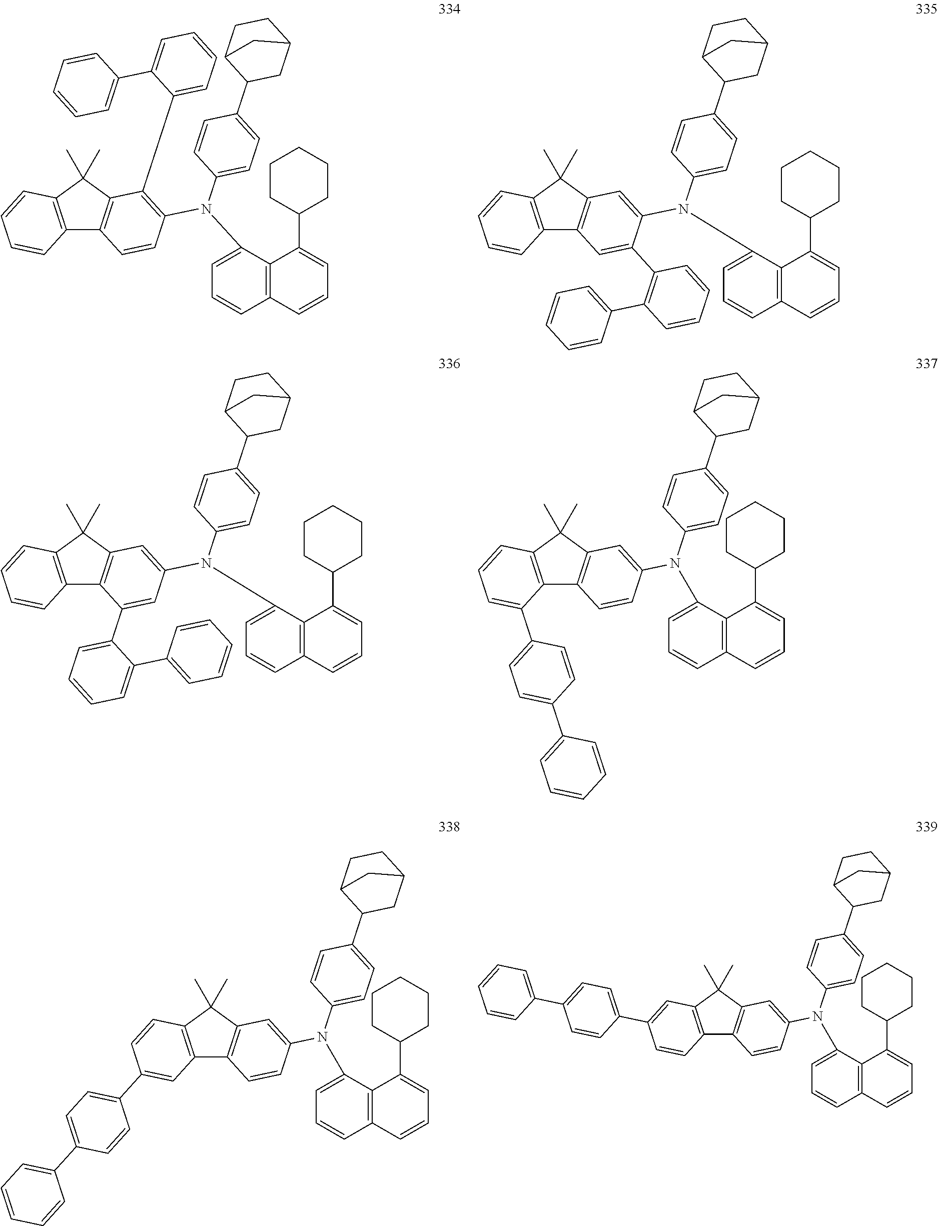

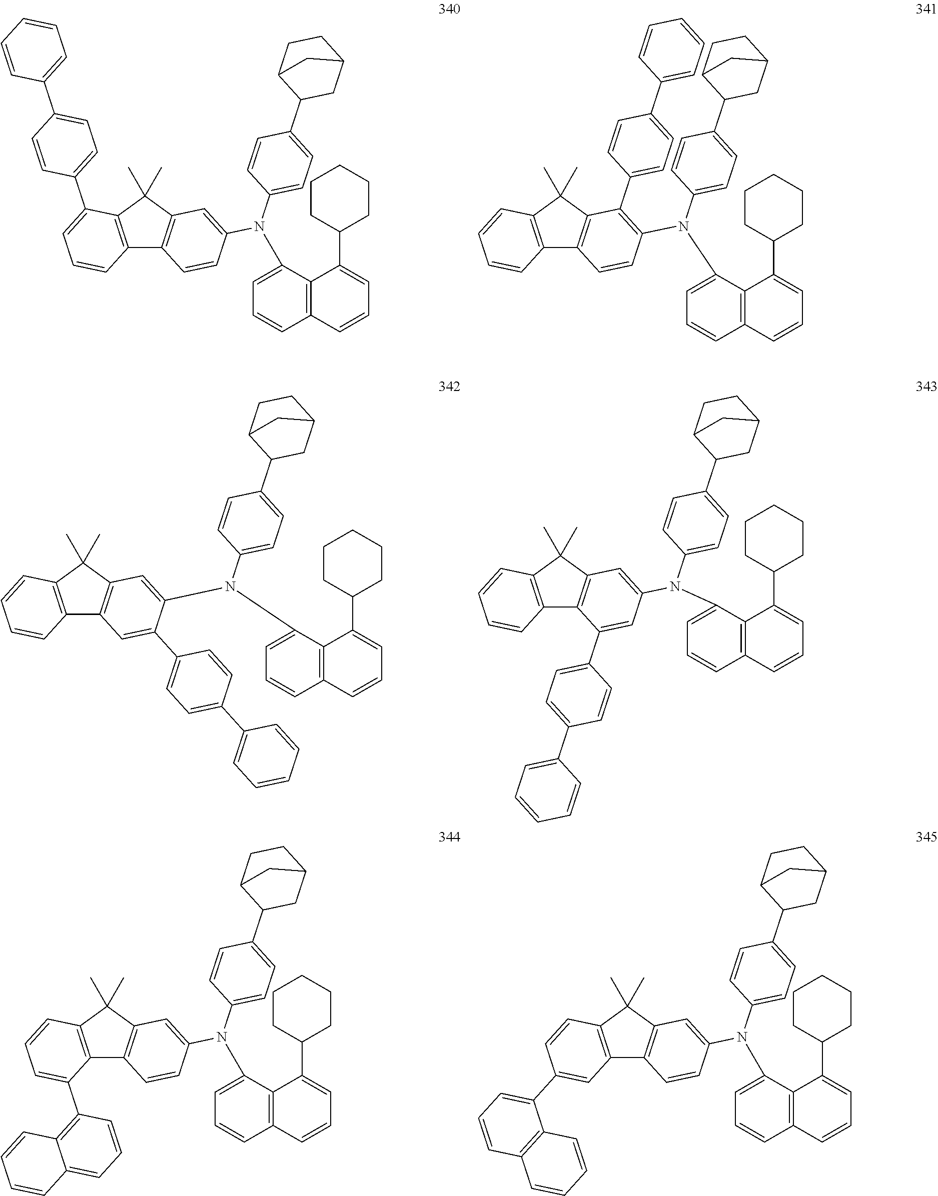

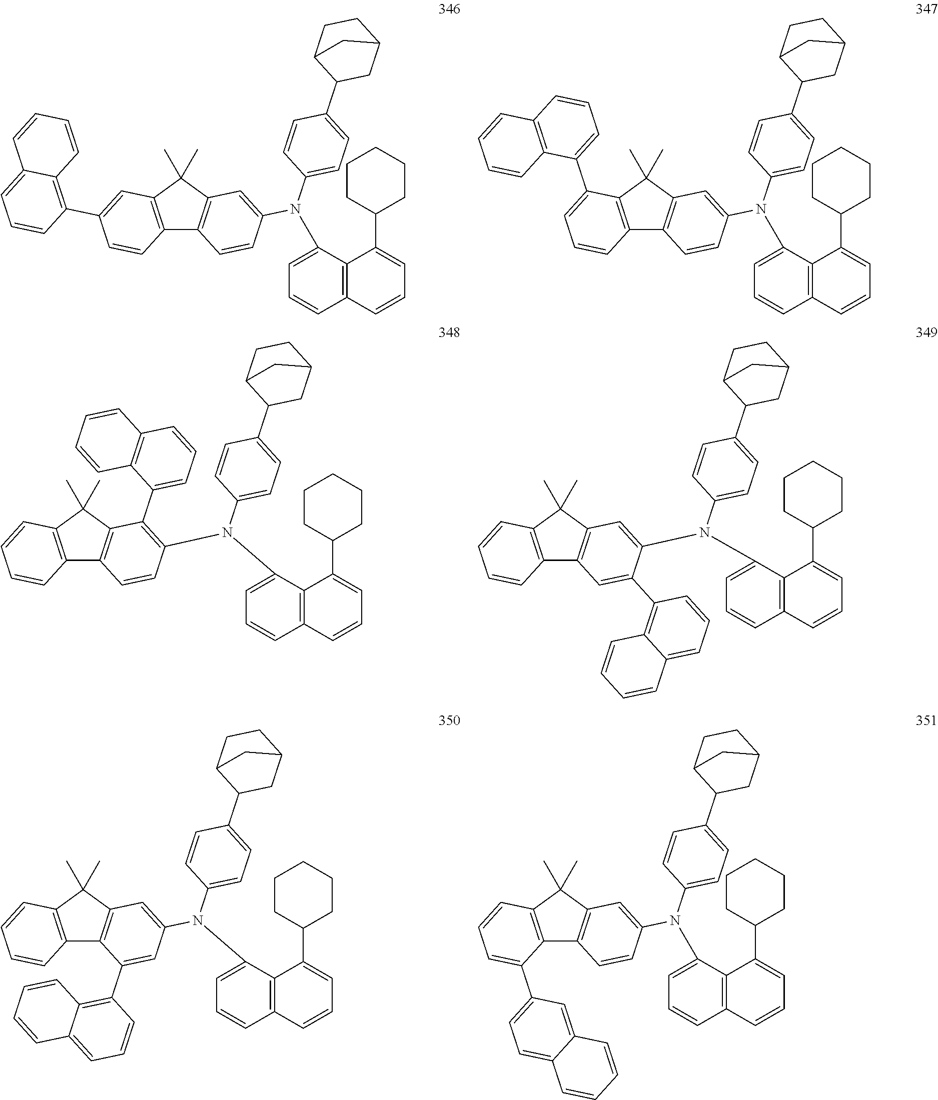

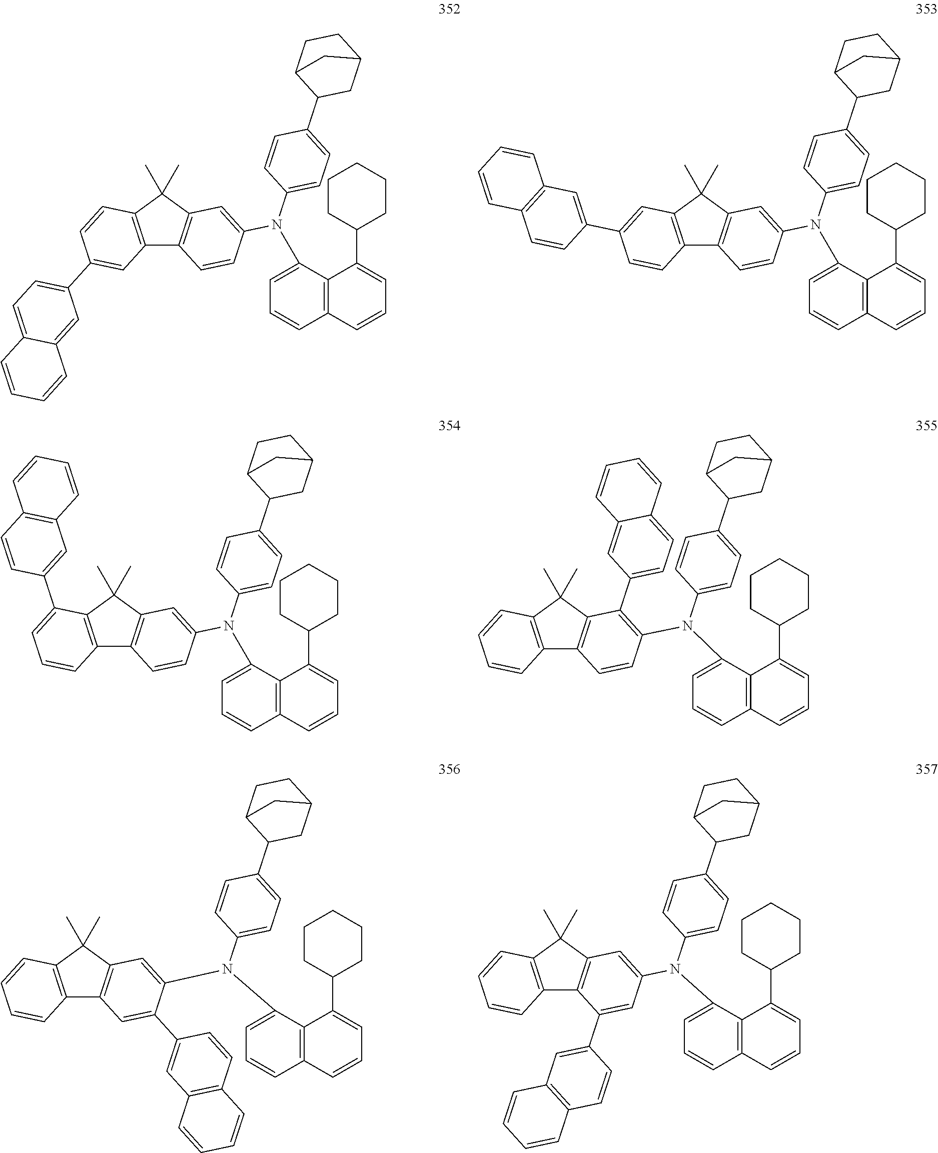

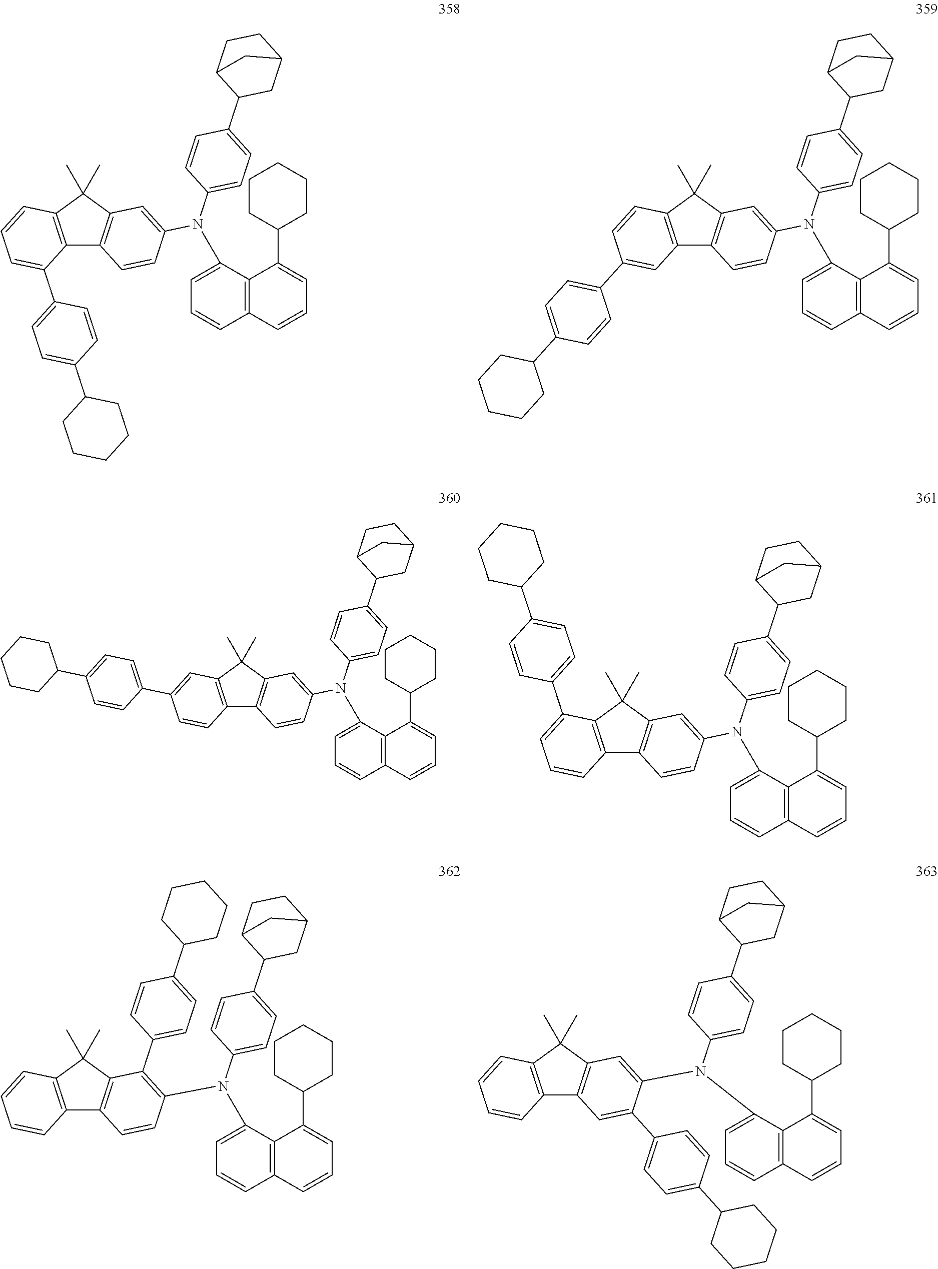

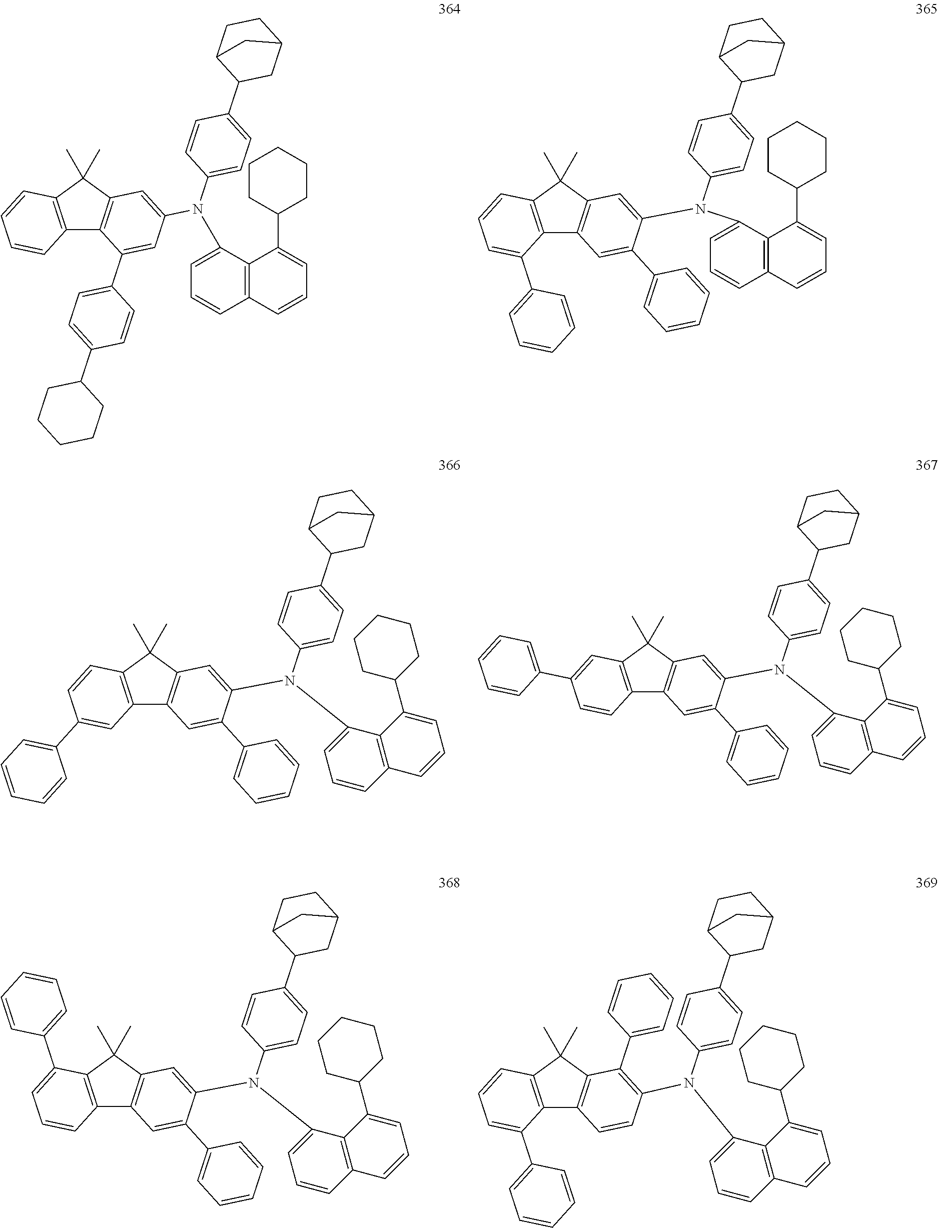

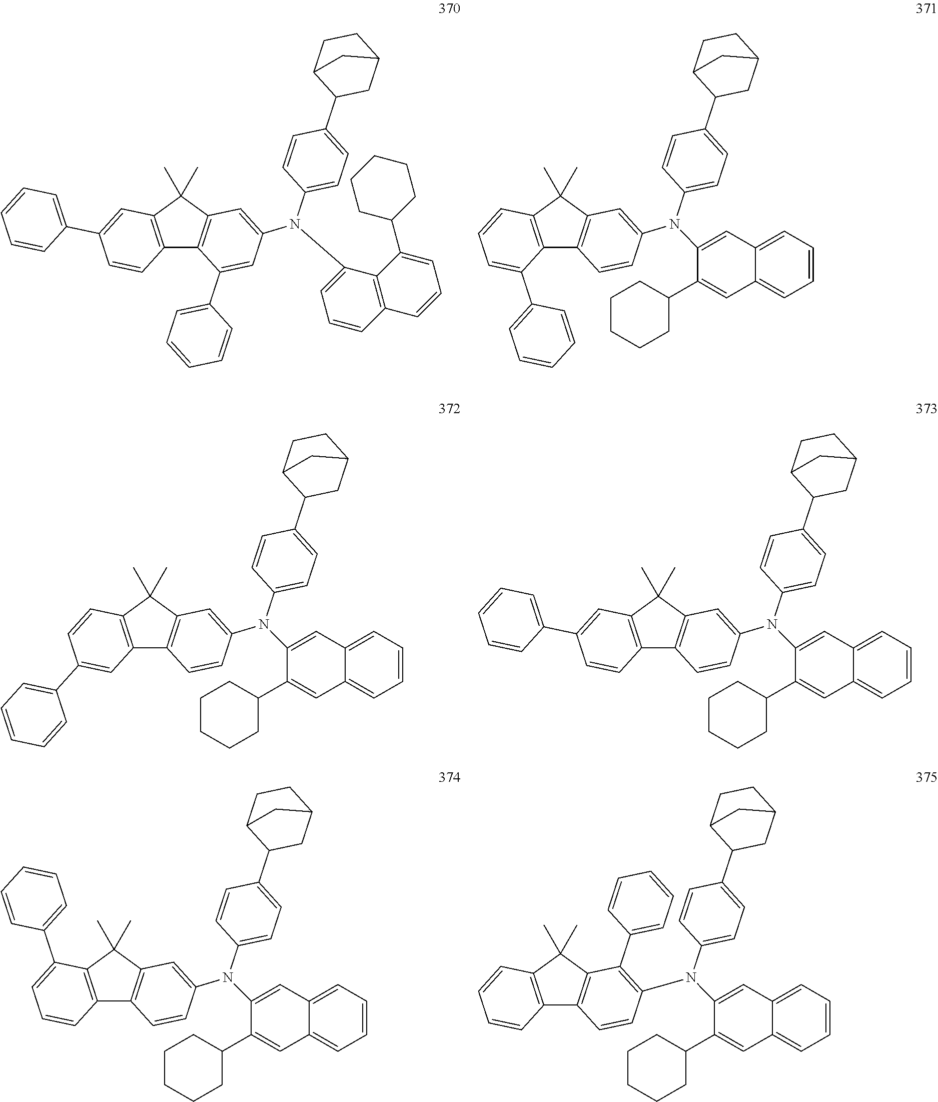

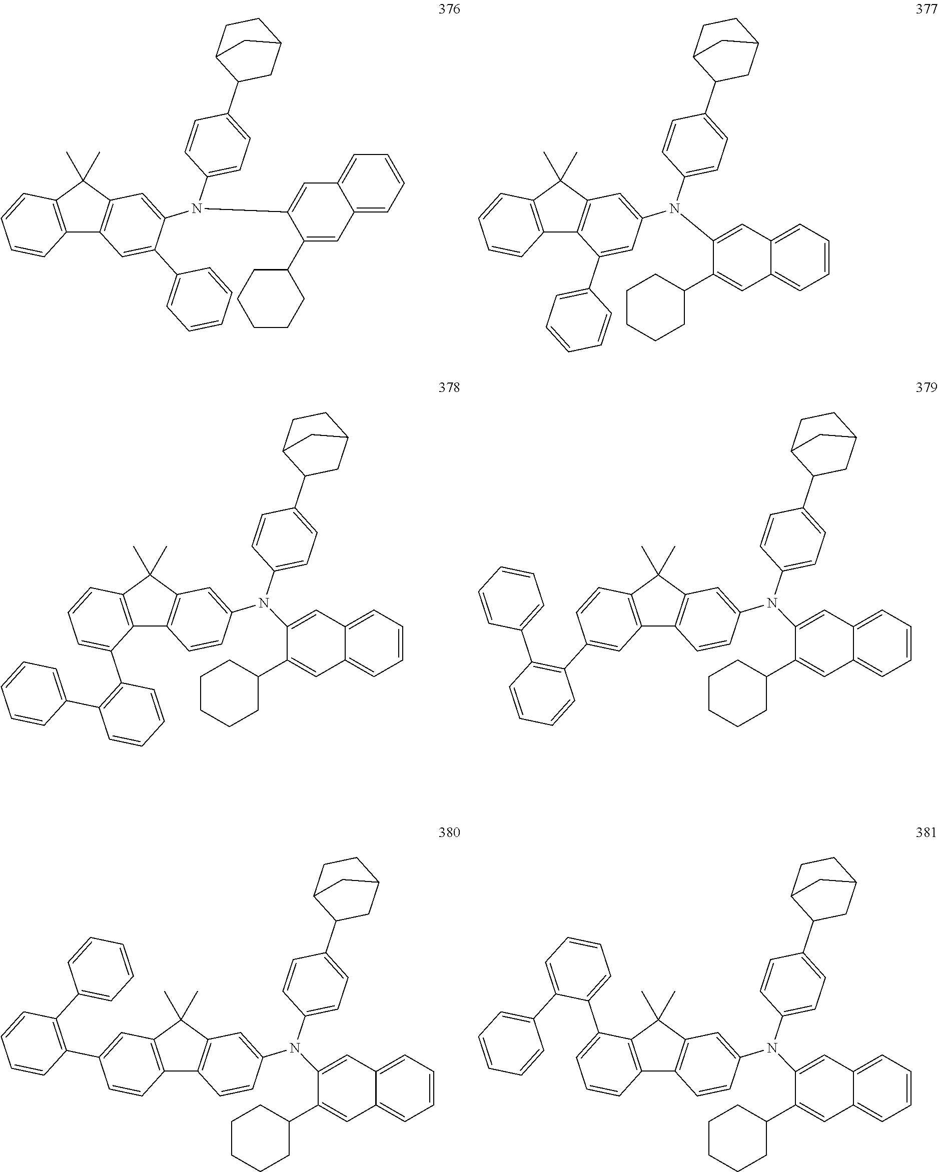

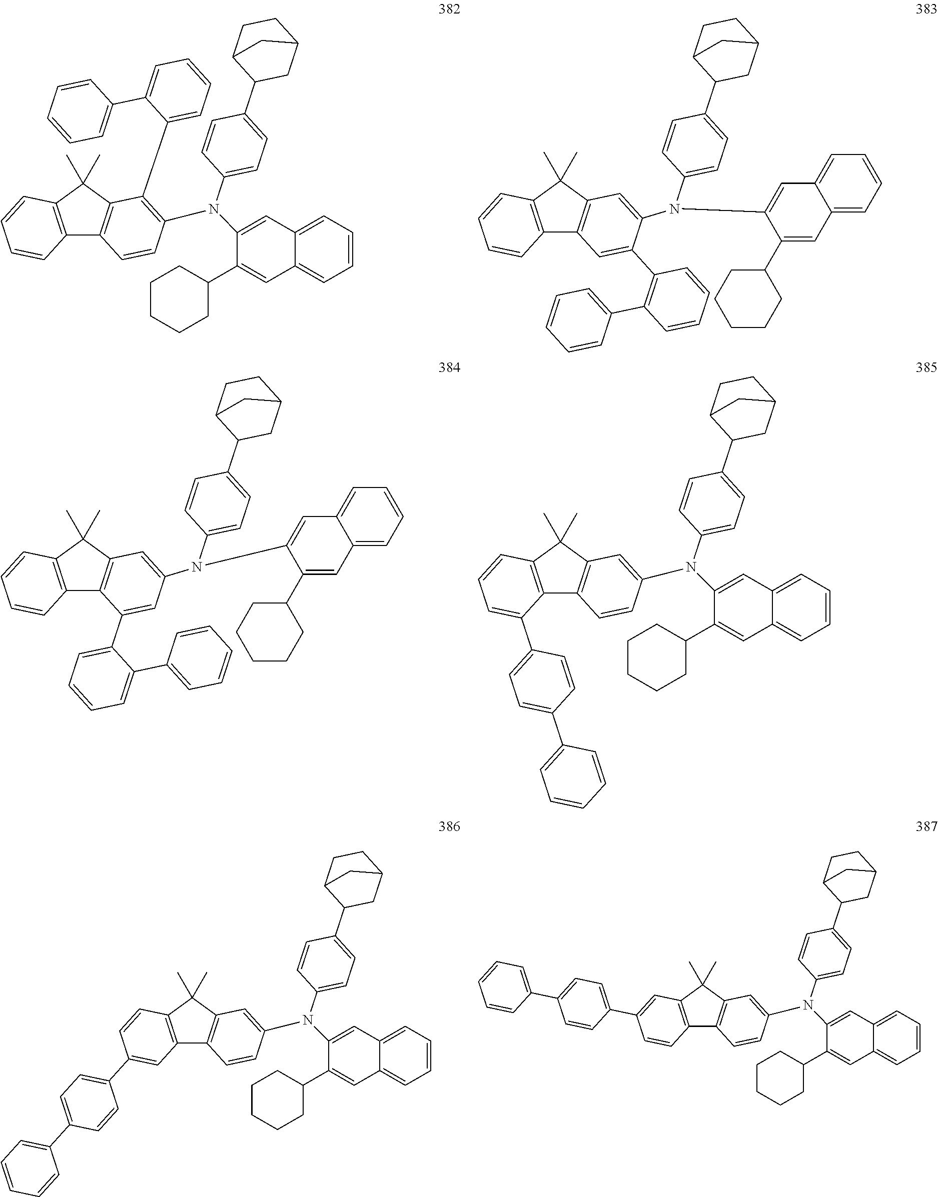

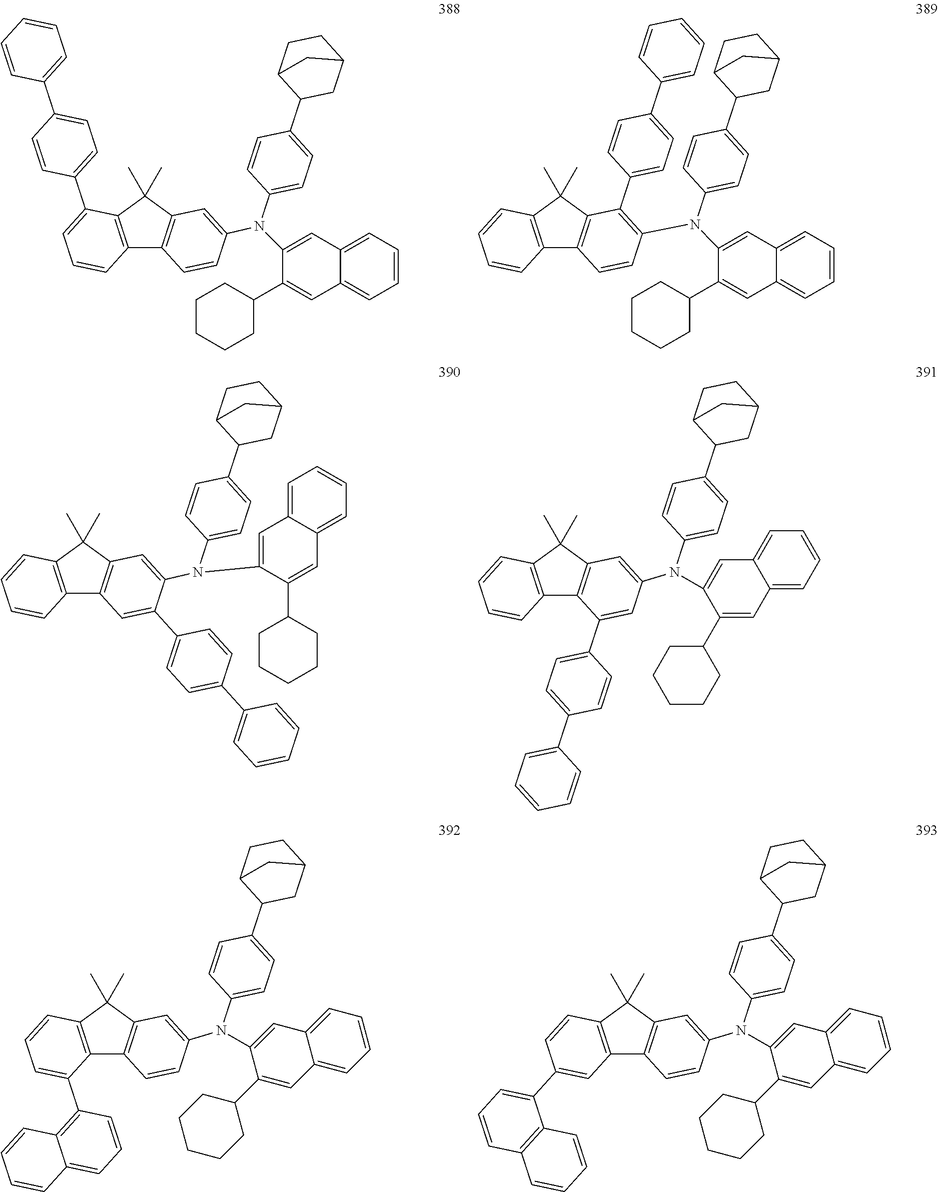

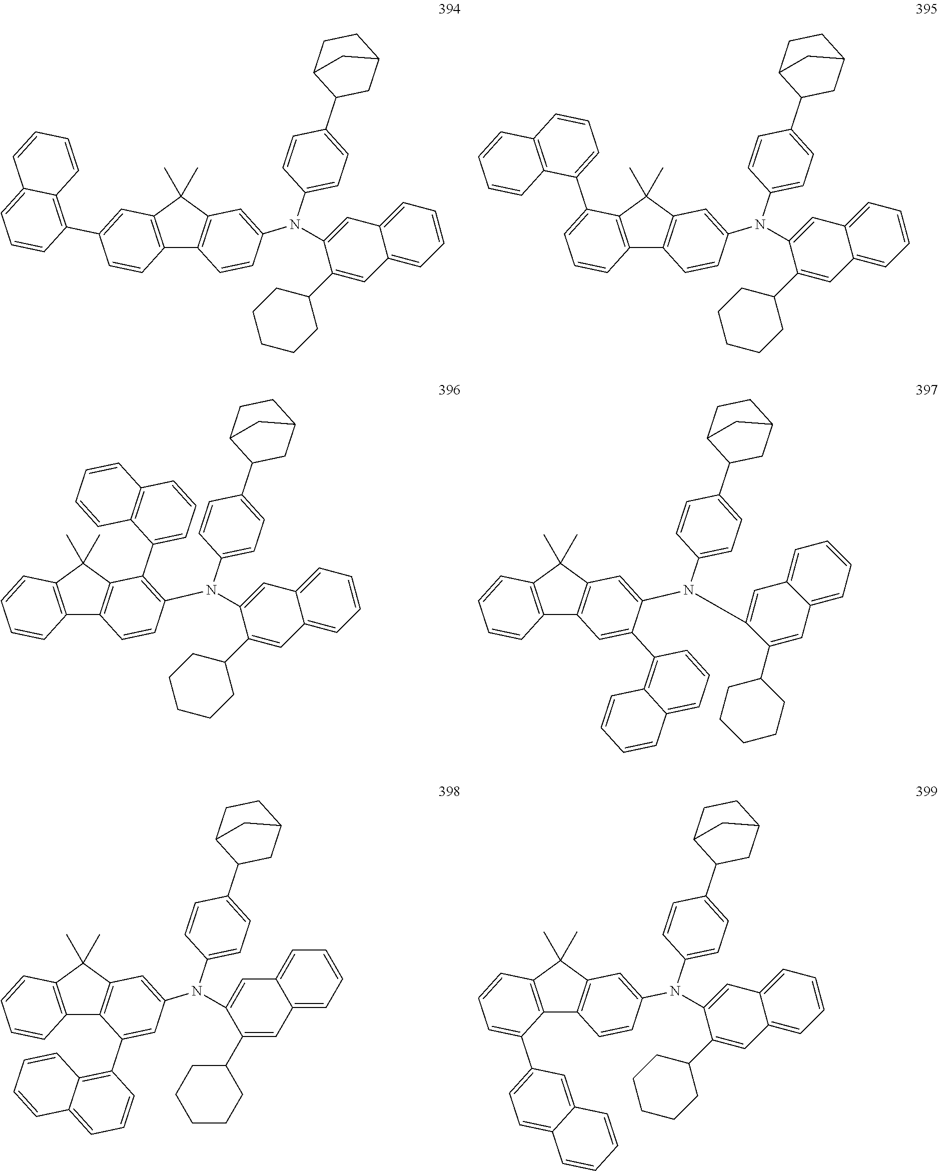

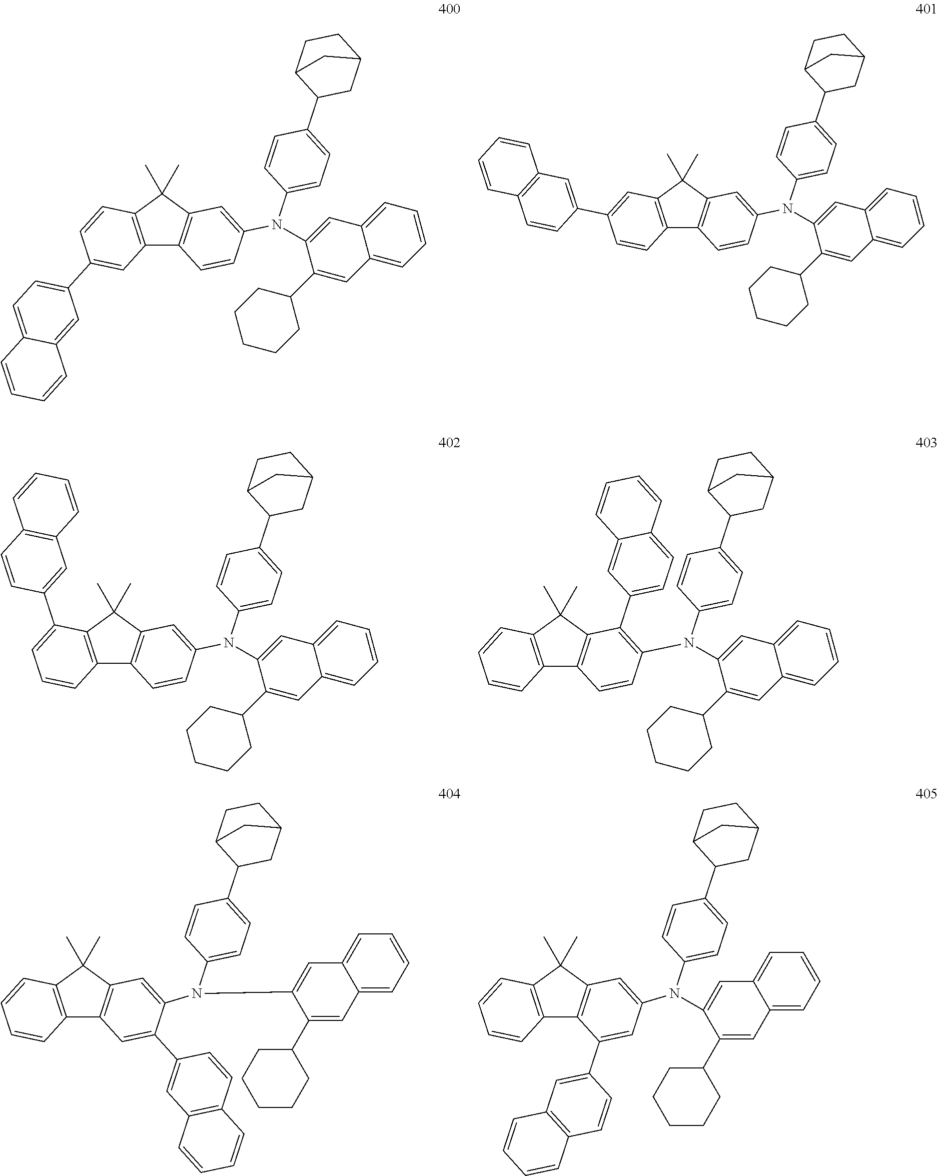

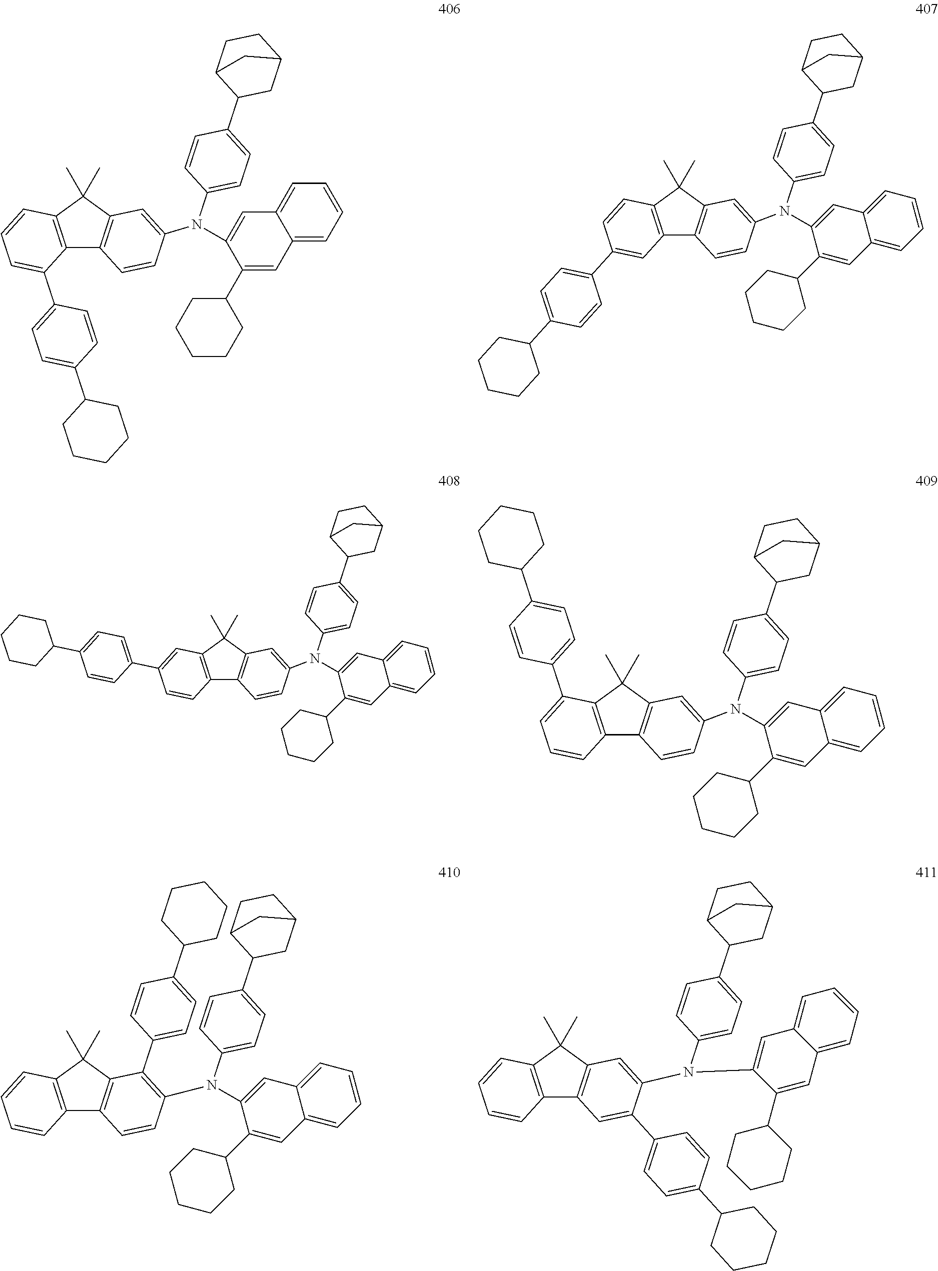

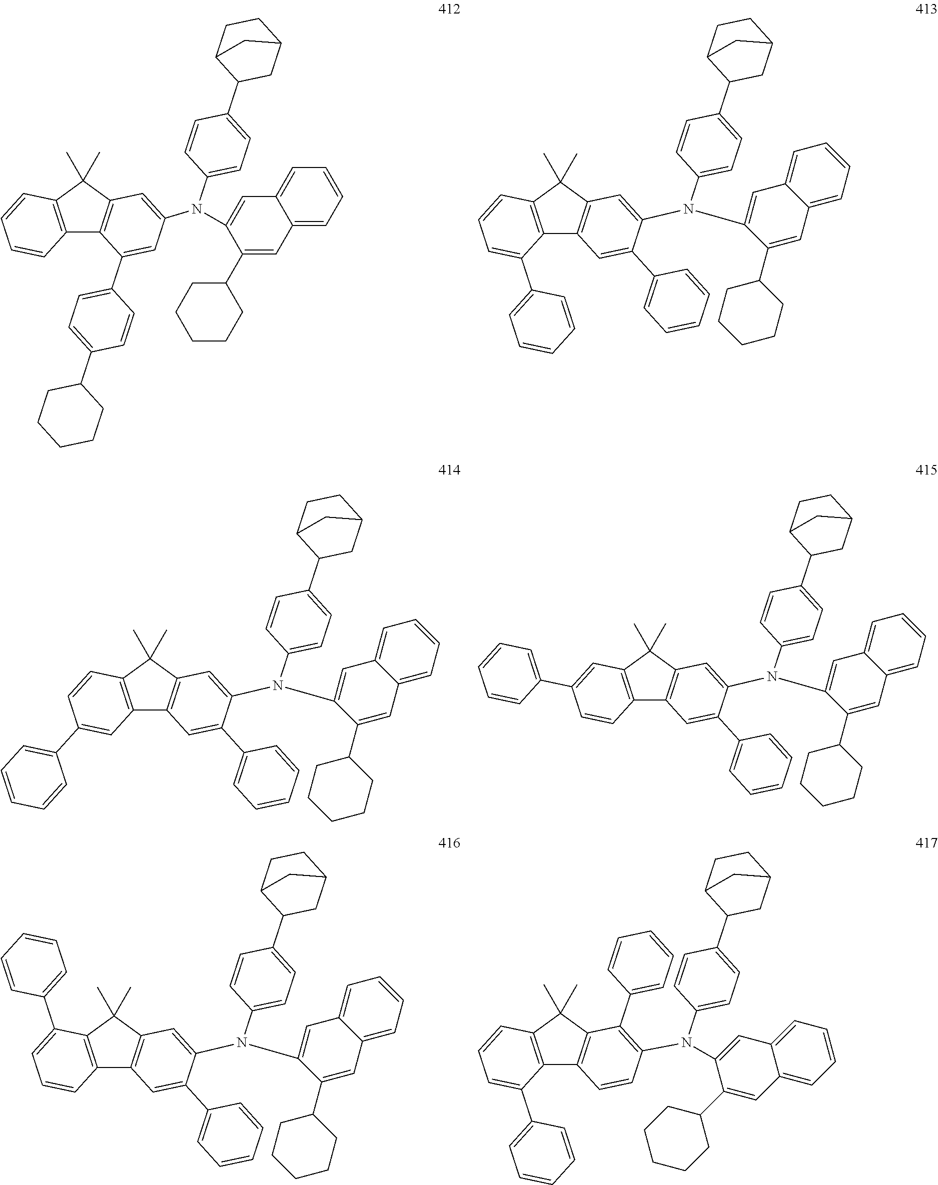

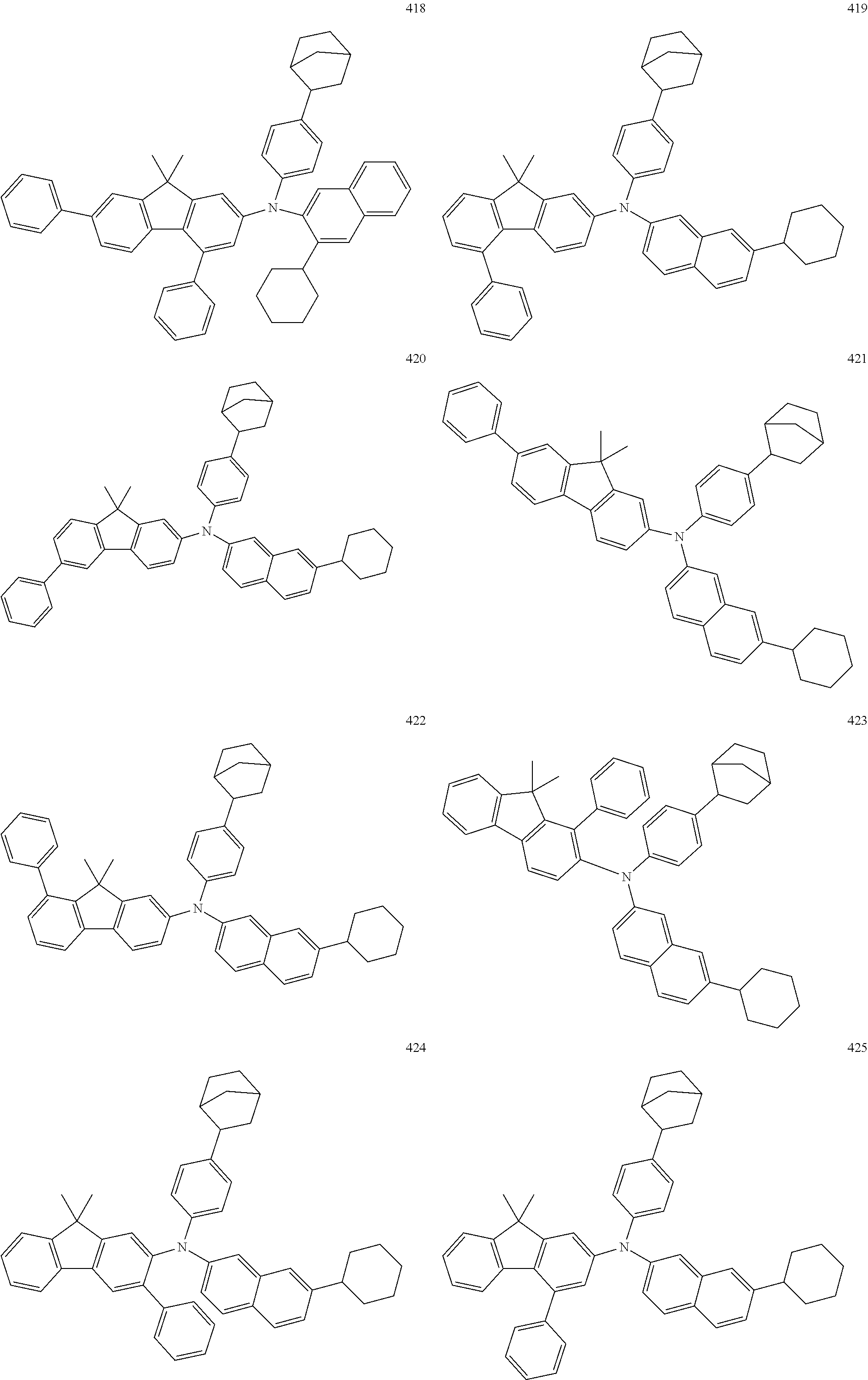

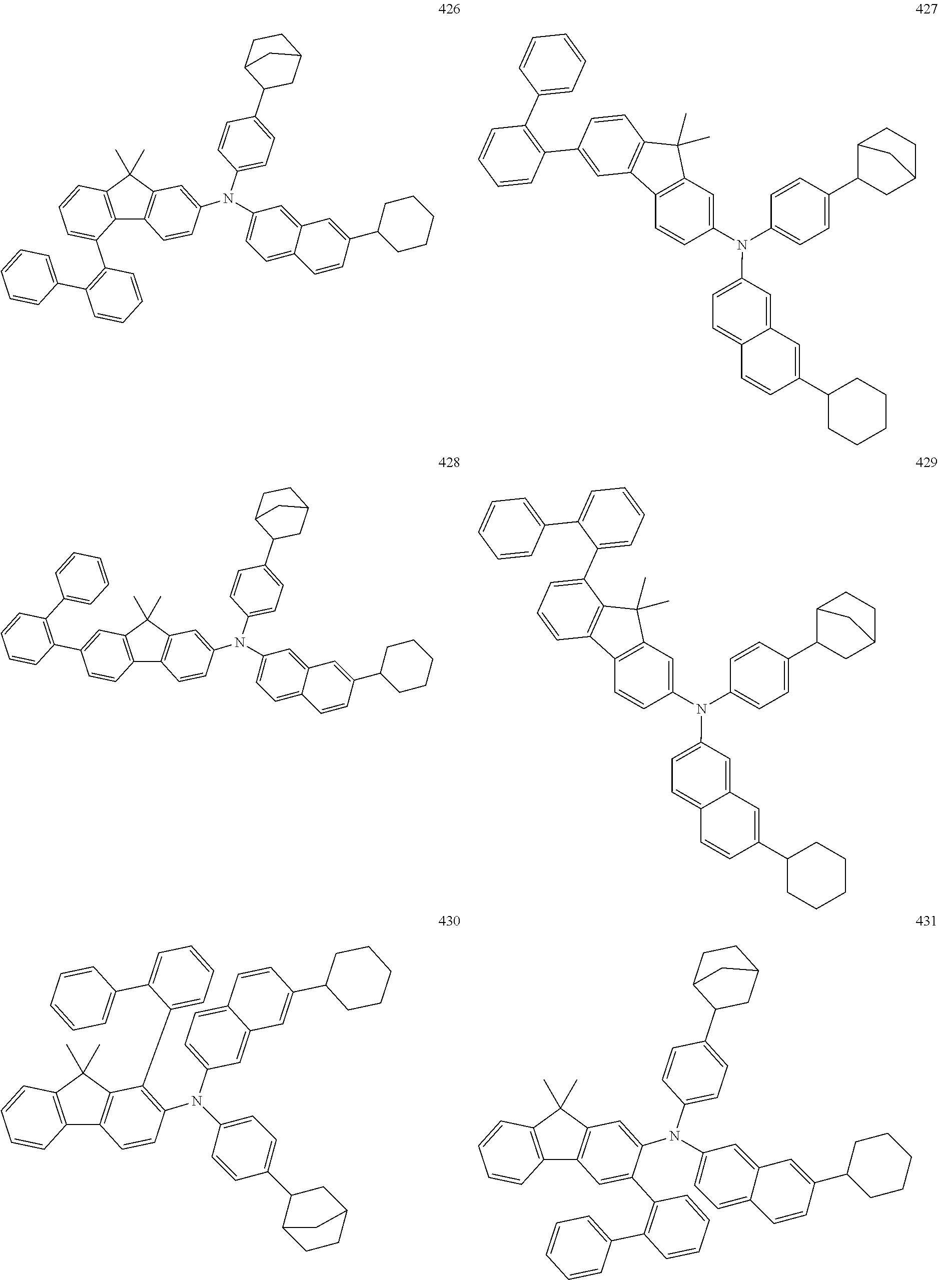

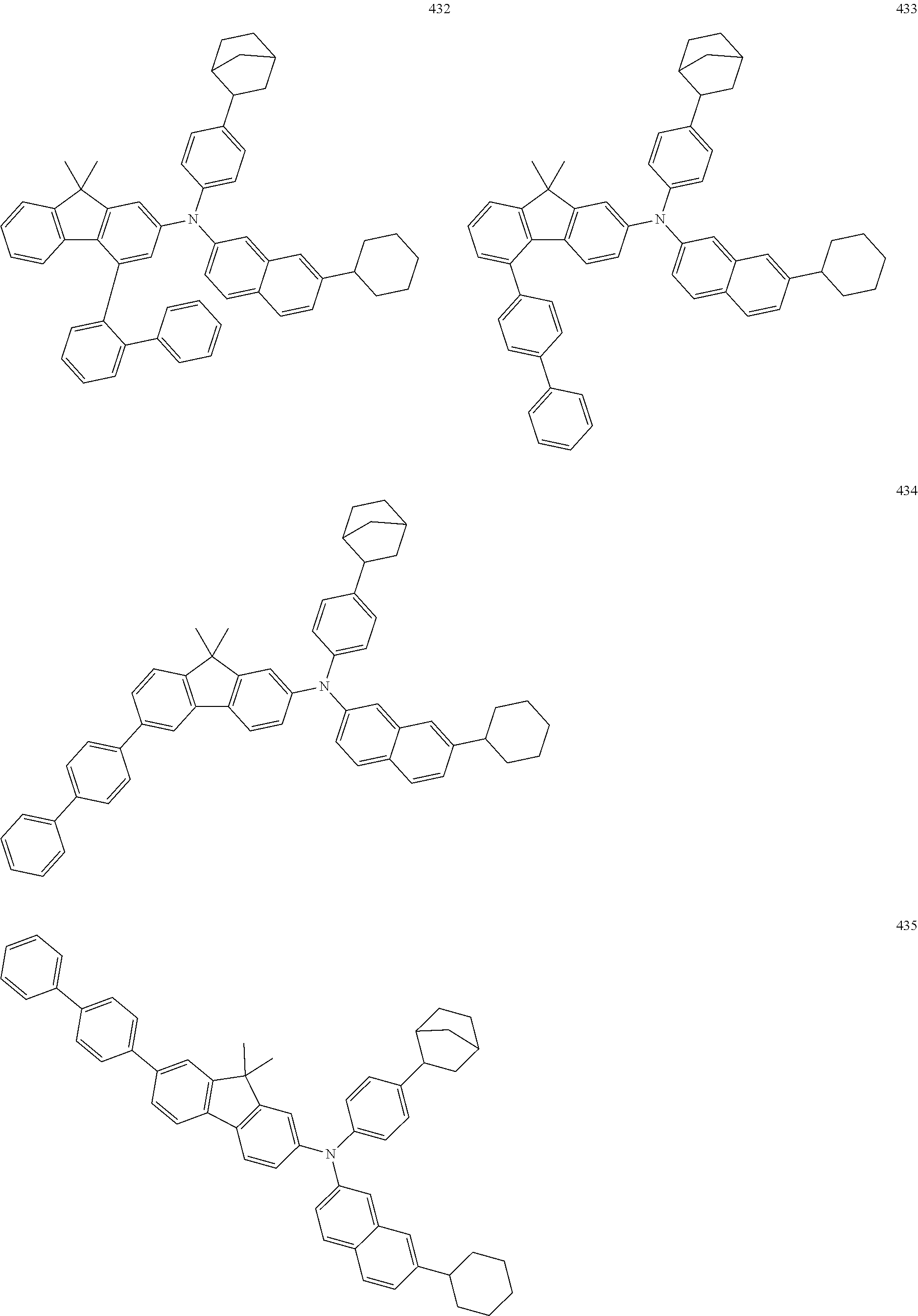 [SMILES: CC1(C)c2ccccc2-c2c(-c3ccccc3-c3ccccc3)cc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3ccc4ccc(C5CCCCC5)cc4c3)cc21 ; CC1(C)c2cc(-c3ccc(-c4ccccc4)cc3)ccc2-c2ccc(N(c3ccc(C4CC5CCC4C5)cc3)c3ccc4ccc(C5CCCCC5)cc4c3)cc21]

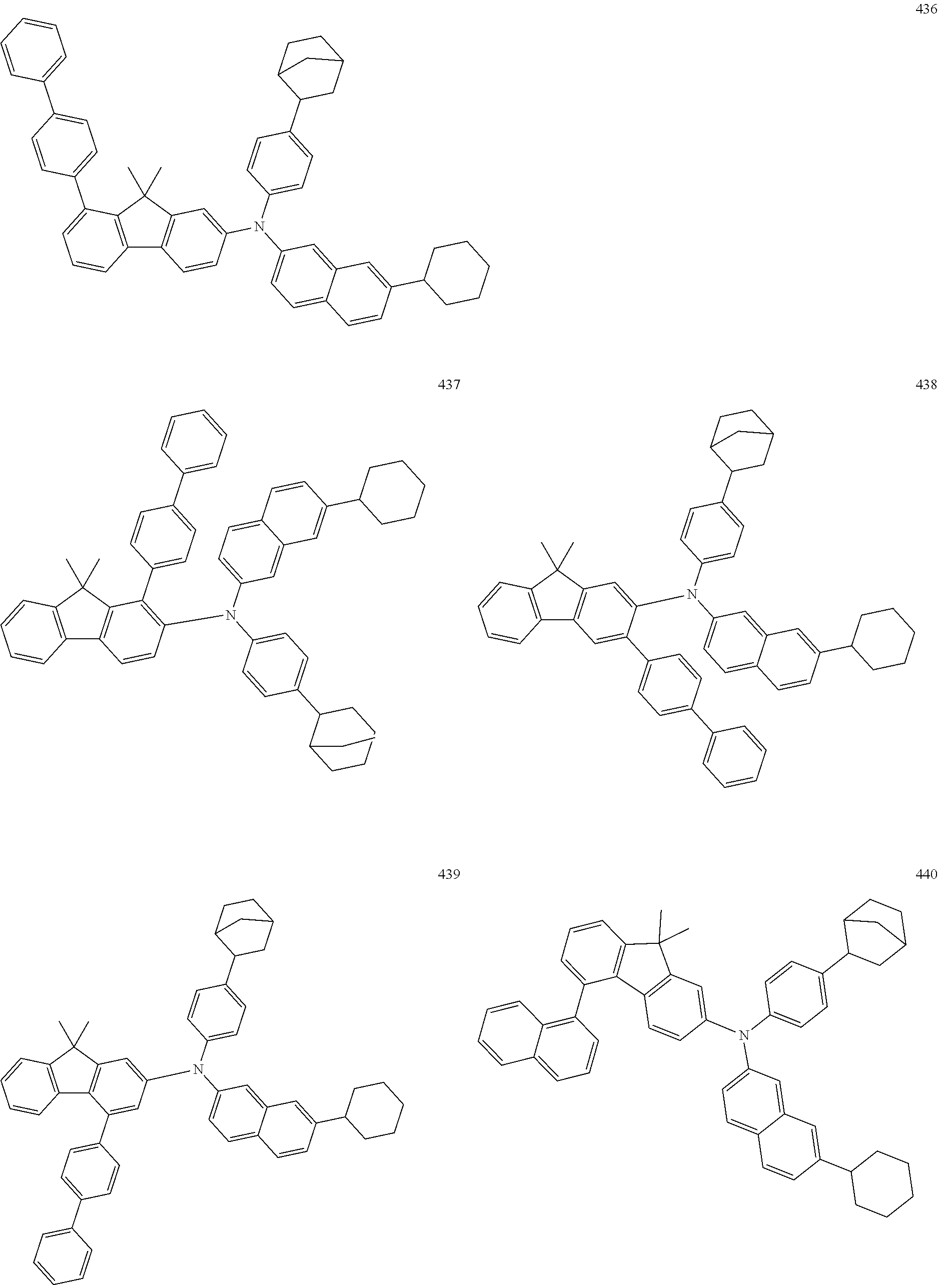 [SMILES: CC1(C)c2cc(N(c3ccc(C4CC5CCC4C5)cc3)c3ccc4ccc(C5CCCCC5)cc4c3)ccc2-c2cccc(-c3ccc(-c4ccccc4)cc3)c21 ; CCC1CCCCC1c1ccc(N(c2ccc3ccc(C4CCCCC4)cc3c2)c2ccc3c(c2-c2ccc(-c4ccccc4)cc2)C(C)(C)c2ccccc2-3)cc1 ; CC1(C)c2ccccc2-c2cc(-c3ccc(-c4ccccc4)cc3)c(N(c3ccc(C4CC5CCC4C5)cc3)c3ccc4ccc(C5CCCCC5)cc4c3)cc21 ; CC1(C)c2ccccc2-c2c(-c3ccc(-c4ccccc4)cc3)cc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2c(-c3cccc4ccccc34)cccc21]

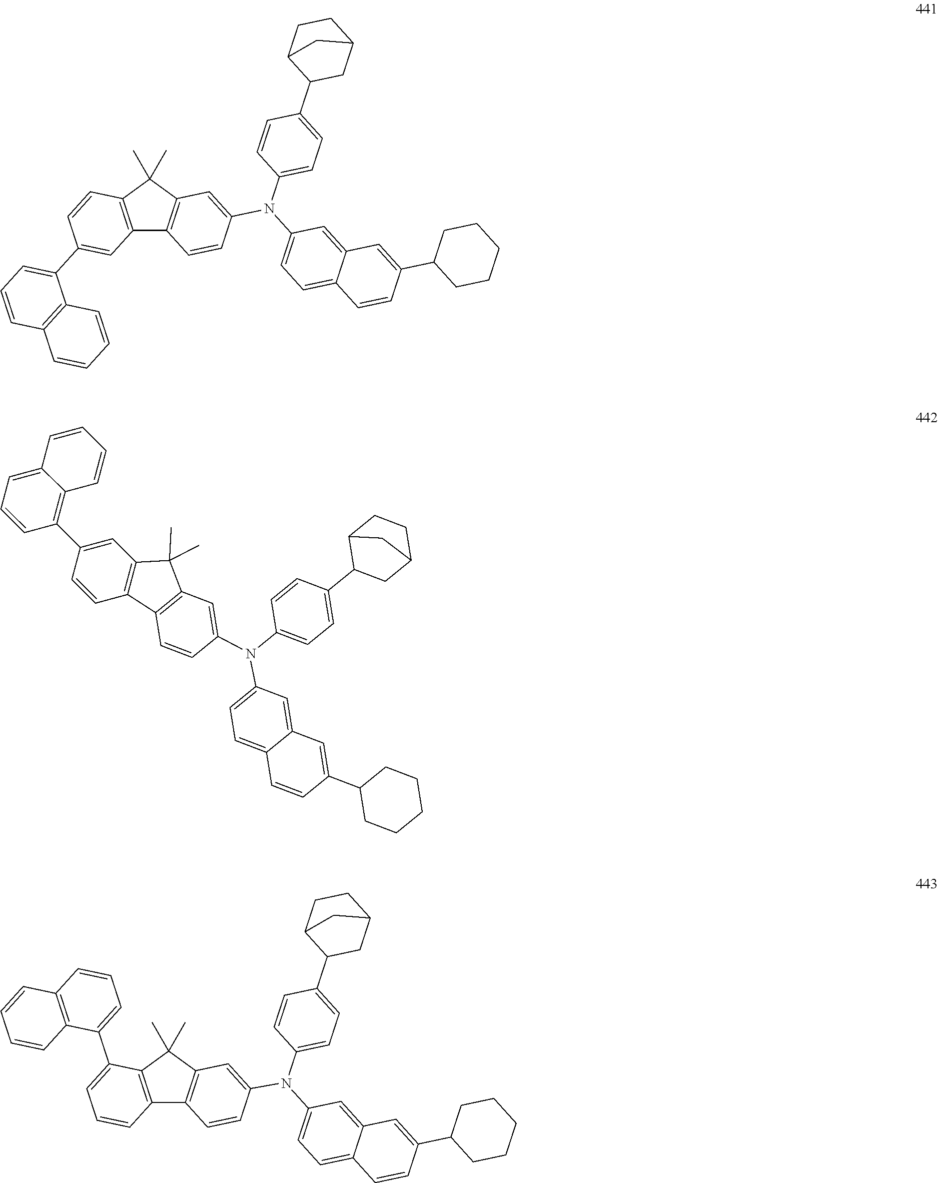 [SMILES: CC1(C)c2ccc(-c3cccc4ccccc34)cc2-c2ccc(N(c3ccc(C4CC5CCC4C5)cc3)c3ccc4ccc(C5CCCCC5)cc4c3)cc21 ; CC1(C)c2cc(-c3cccc4ccccc34)ccc2-c2ccc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2cccc(-c3cccc4ccccc34)c21]

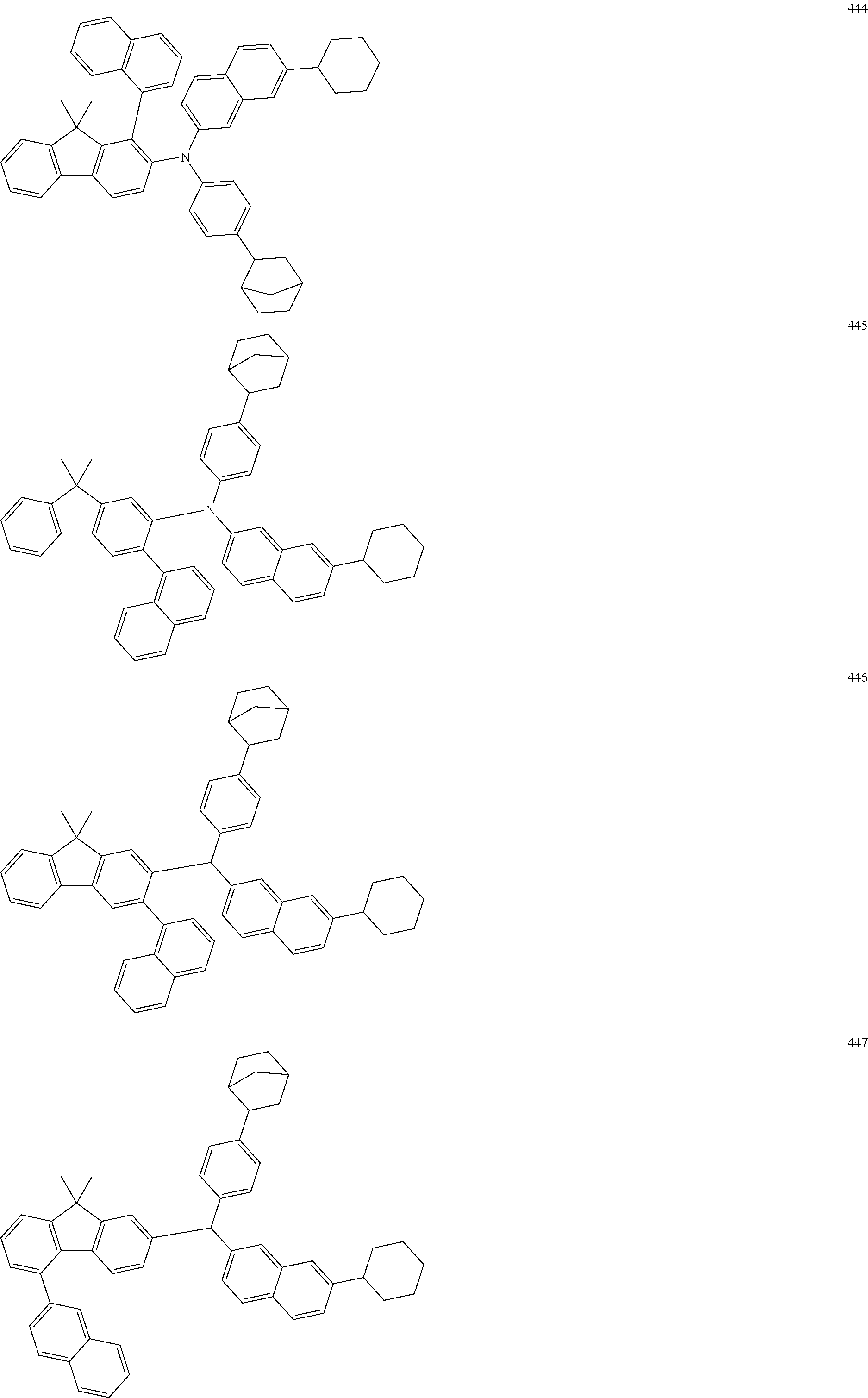

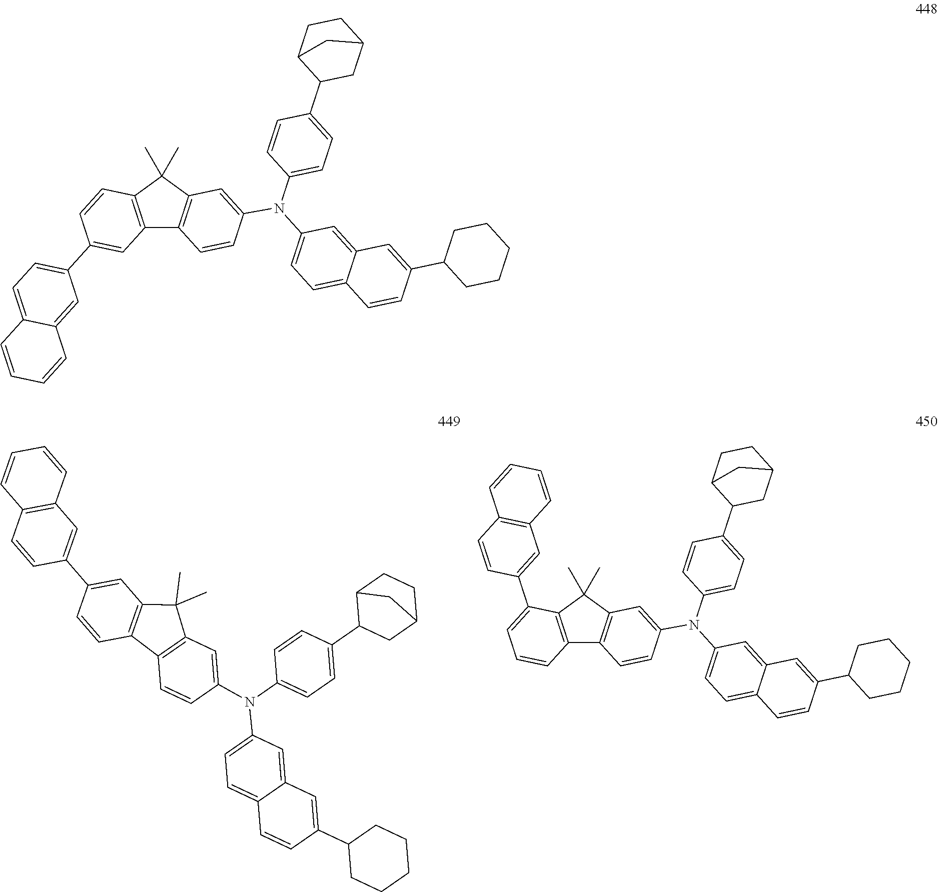 [SMILES: CC1(C)c2ccc(-c3ccc4ccccc4c3)cc2-c2ccc(N(c3ccc(C4CC5CCC4C5)cc3)c3ccc4ccc(C5CCCCC5)cc4c3)cc21 ; CC1(C)c2cc(-c3ccc4ccccc4c3)ccc2-c2ccc(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)ccc2-c2cccc(-c3ccc4ccccc4c3)c21]

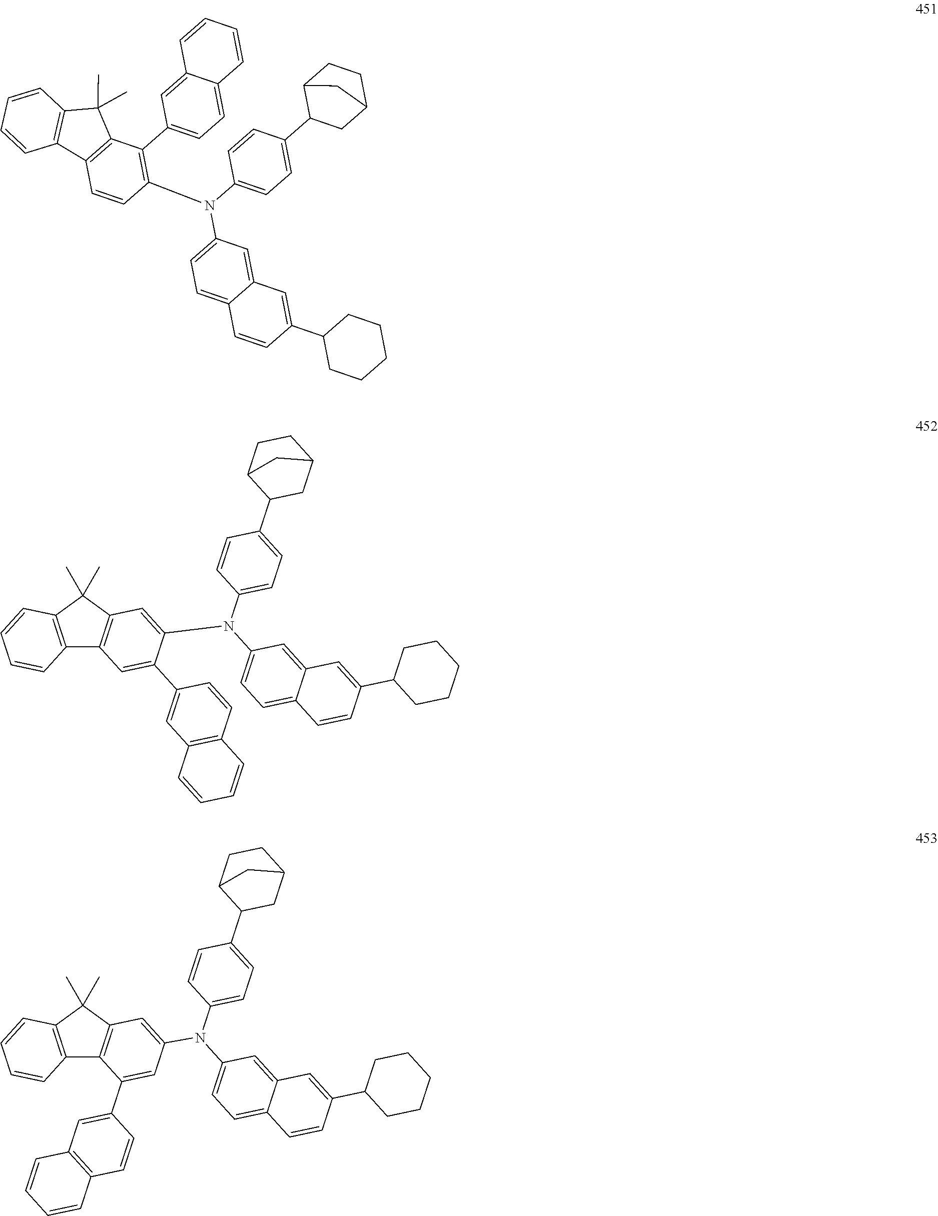 [SMILES: CC1(C)c2ccccc2-c2ccc(N(c3ccc(C4CC5CCC4C5)cc3)c3ccc4ccc(C5CCCCC5)cc4c3)c(-c3ccc4ccccc4c3)c21 ; CC1(C)c2ccccc2-c2cc(-c3ccc4ccccc4c3)c(N(c3ccc(C4CC5CCC4C5)cc3)c3ccc4ccc(C5CCCCC5)cc4c3)cc21 ; CC1(C)c2ccccc2-c2c(-c3ccc4ccccc4c3)cc(N(c3ccc(C4CC5CCC4C5)cc3)c3ccc4ccc(C5CCCCC5)cc4c3)cc21]

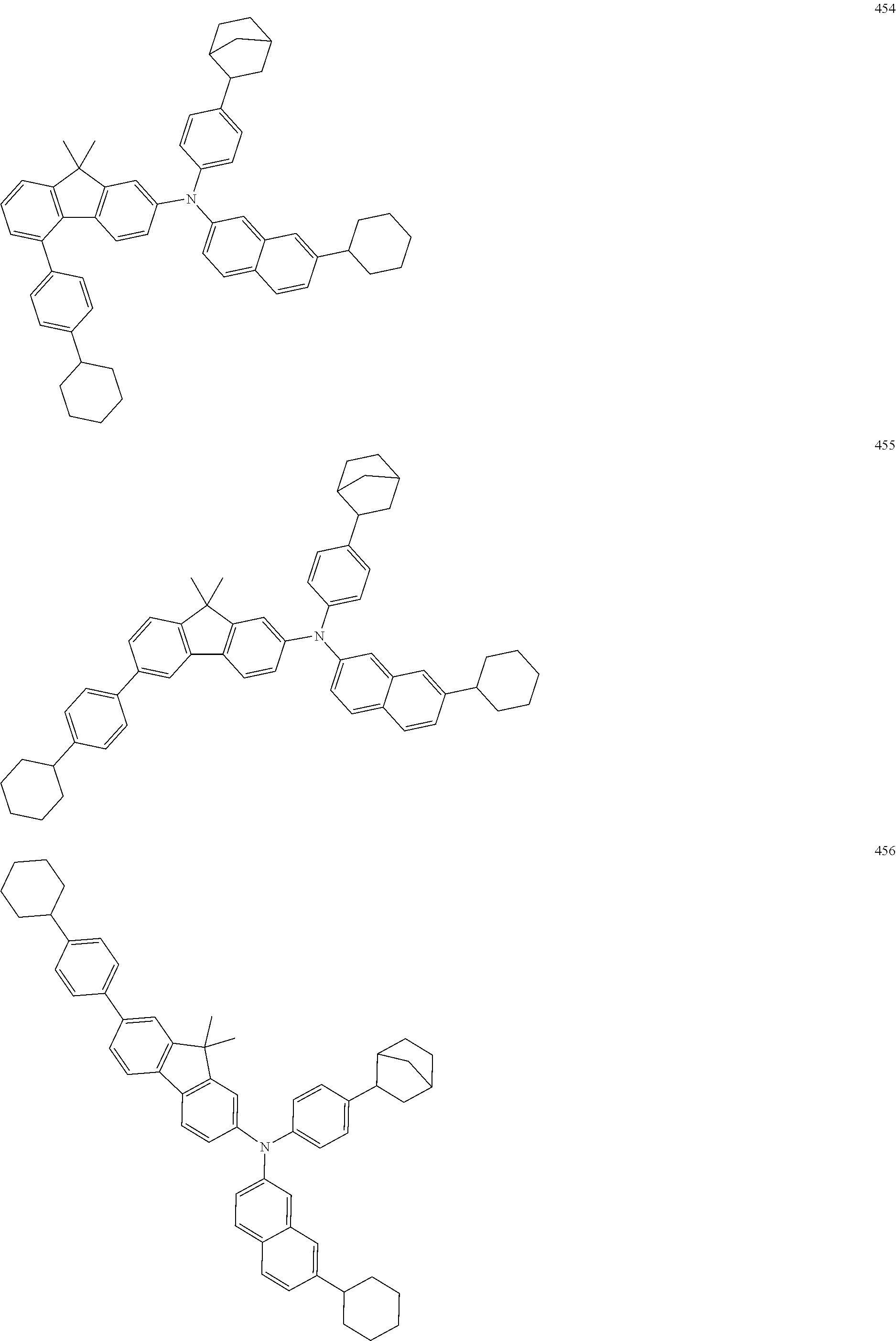

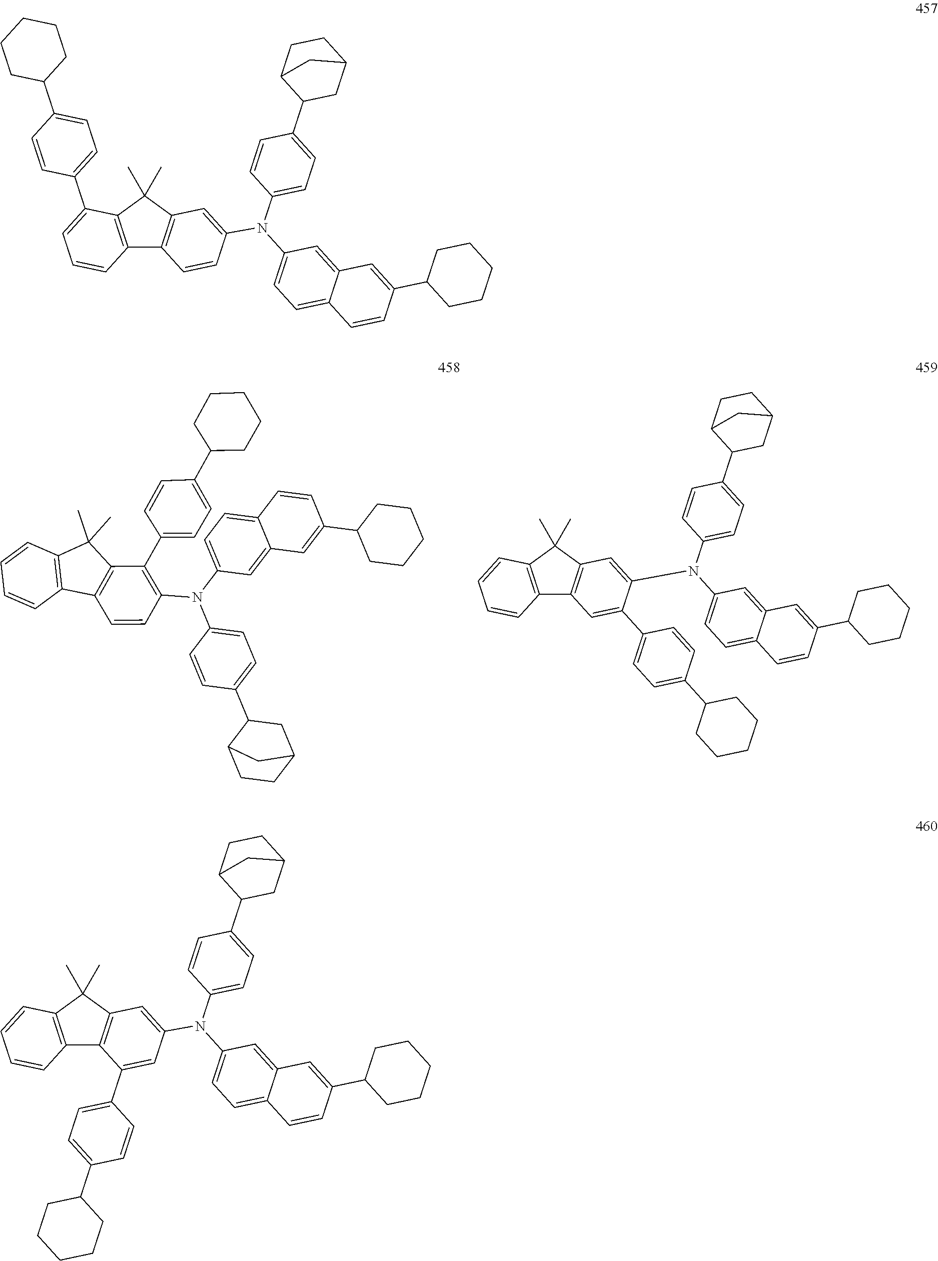 [SMILES: CC1(C)c2cc(N(c3ccc(C4CC5CCC4C5)cc3)c3ccc4ccc(C5CCCCC5)cc4c3)ccc2-c2cccc(-c3ccc(C4CCCCC4)cc3)c21 ; CC1(C)c2ccccc2-c2ccc(N(c3ccc(C4CC5CCC4C5)cc3)c3ccc4ccc(C5CCCCC5)cc4c3)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3ccc4ccc(C5CCCCC5)cc4c3)cc21 ; CC1(C)c2ccccc2-c2c(-c3ccc(C4CCCCC4)cc3)cc(N(c3ccc(C4CC5CCC4C5)cc3)c3ccc4ccc(C5CCCCC5)cc4c3)cc21]

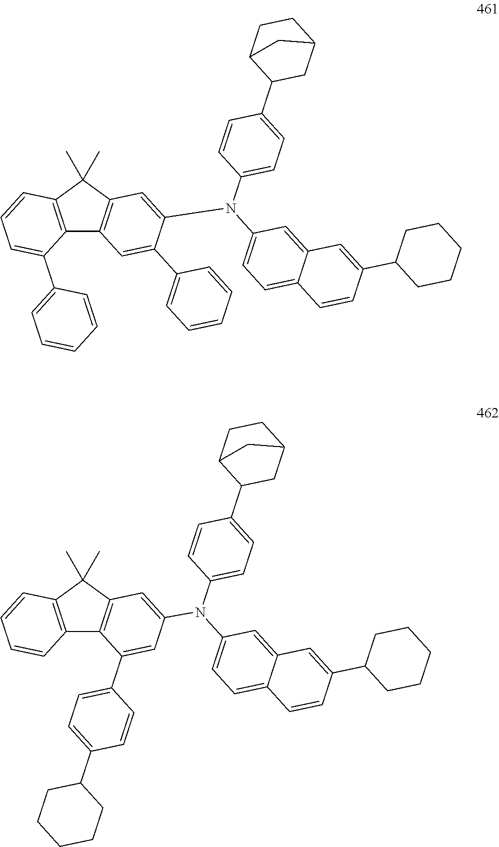 [SMILES: CC1(C)c2cc(N(c3ccc(C4CC5CCC4C5)cc3)c3ccc4ccc(C5CCCCC5)cc4c3)c(-c3ccccc3)cc2-c2c(-c3ccccc3)cccc21 ; CC1(C)c2ccccc2-c2c(-c3ccc(C4CCCCC4)cc3)cc(N(c3ccc(C4CC5CCC4C5)cc3)c3ccc4ccc(C5CCCCC5)cc4c3)cc21]

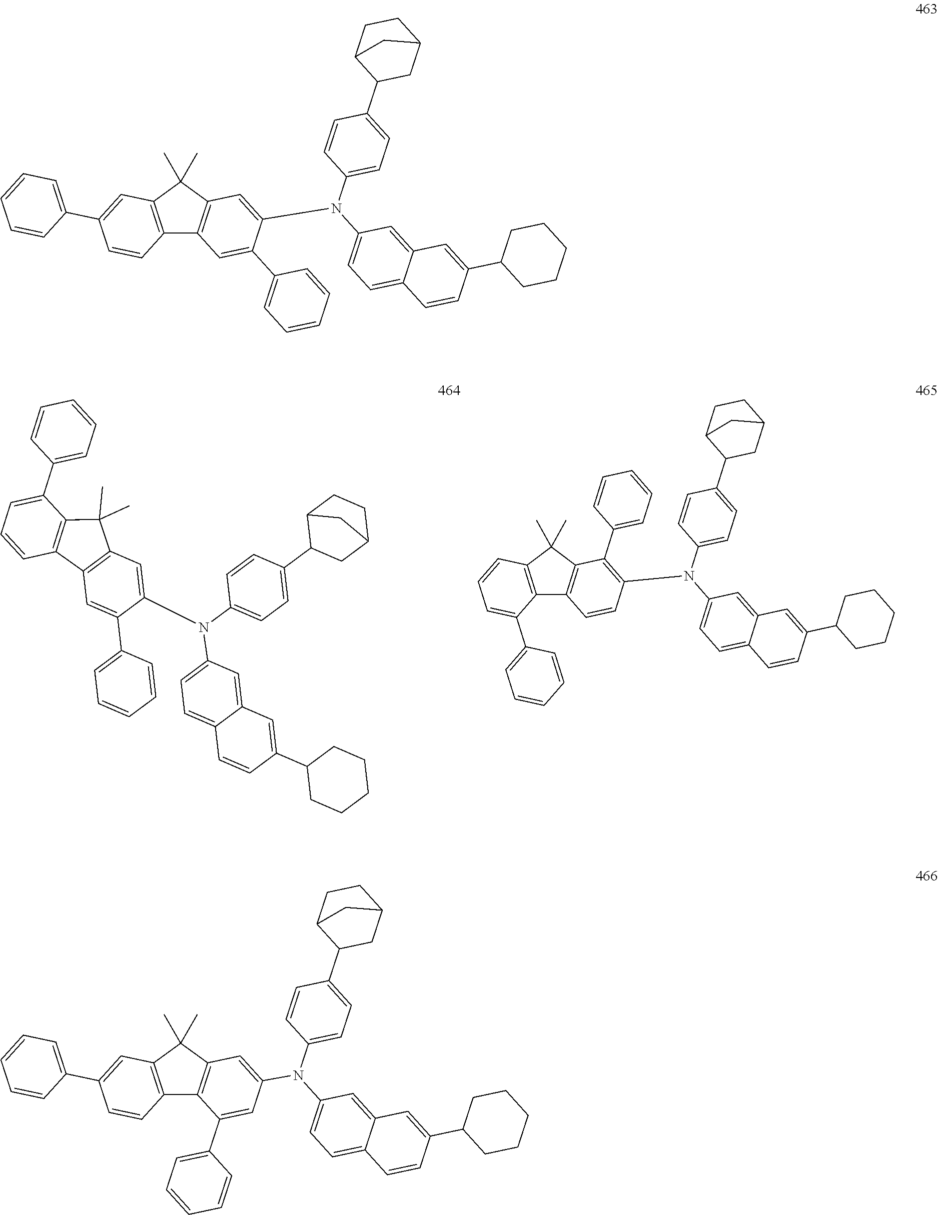 [SMILES: CC1(C)c2cc(-c3ccccc3)ccc2-c2cc(-c3ccccc3)c(N(c3ccc(C4CC5CCC4C5)cc3)c3ccc4ccc(C5CCCCC5)cc4c3)cc21 ; CC1(C)c2cc(N(c3ccc(C4CC5CCC4C5)cc3)c3ccc4ccc(C5CCCCC5)cc4c3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3ccc4ccc(C5CCCCC5)cc4c3)c(-c3ccccc3)c21 ; CC1(C)c2cc(-c3ccccc3)ccc2-c2c(-c3ccccc3)cc(N(c3ccc(C4CC5CCC4C5)cc3)c3ccc4ccc(C5CCCCC5)cc4c3)cc21]

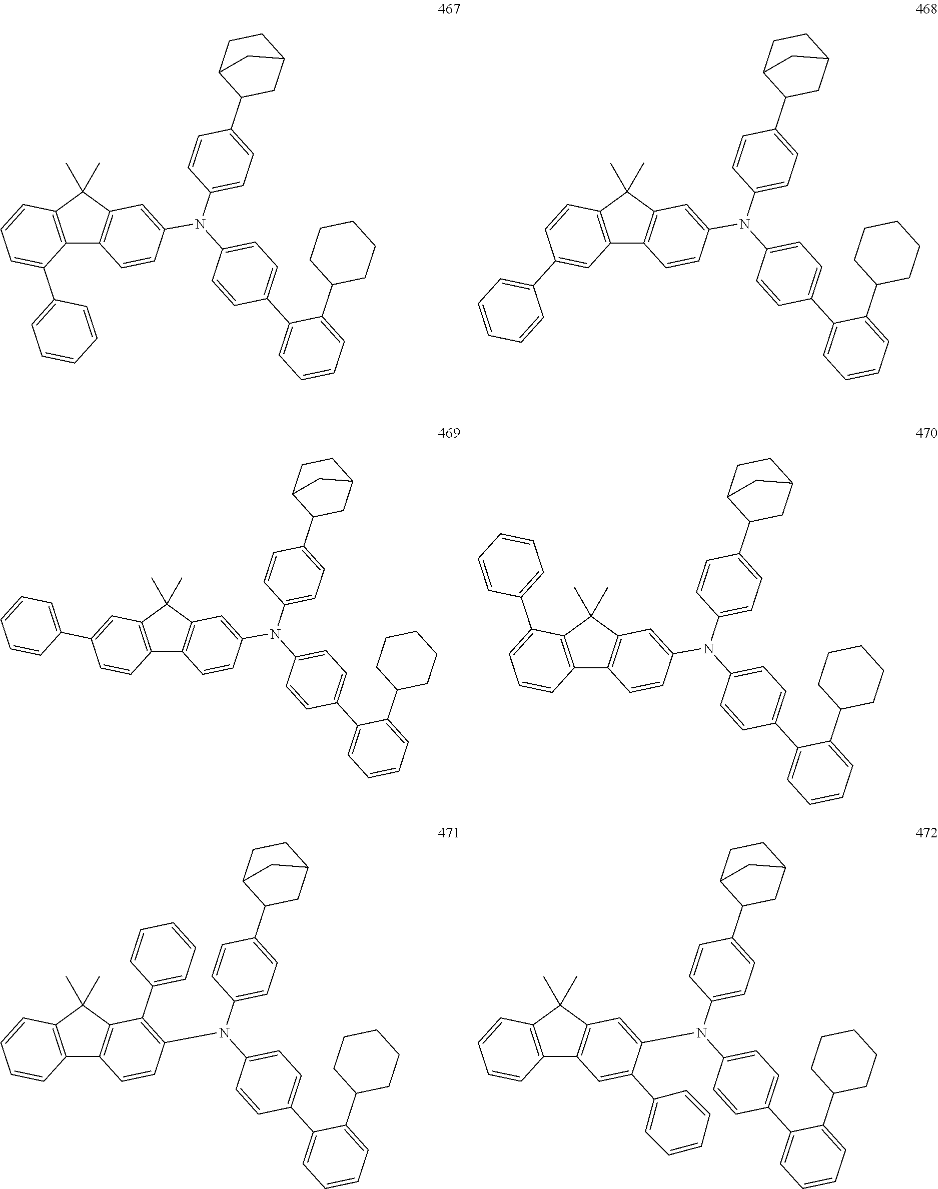

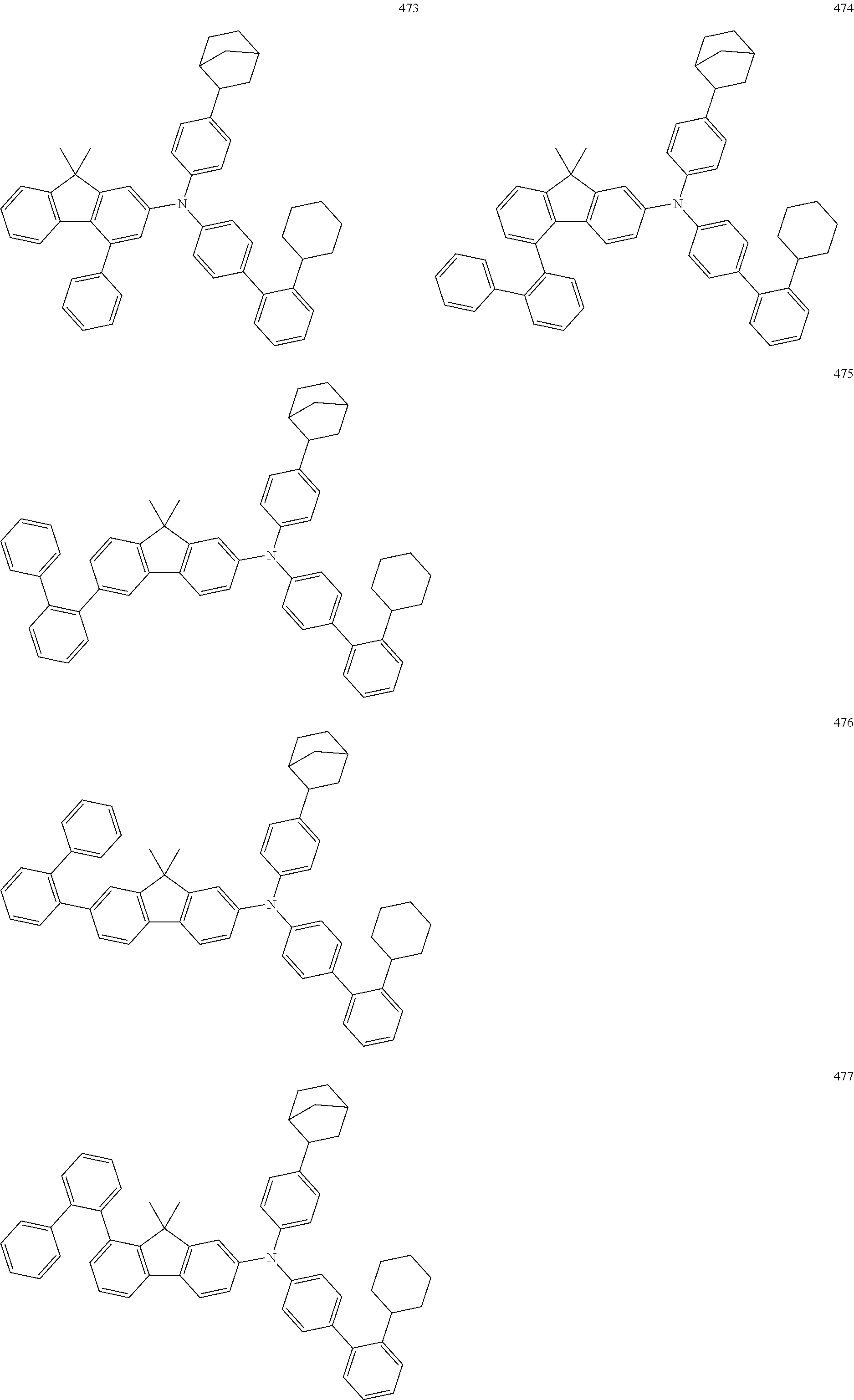 [SMILES: CC1(C)c2ccccc2-c2c(-c3ccccc3)cc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2c(-c3ccccc3-c3ccccc3)cccc21 ; CC1(C)c2ccc(-c3ccccc3-c3ccccc3)cc2-c2ccc(N(c3ccc(-c4ccccc4C4CCCCC4)cc3)c3ccc(C4CC5CCC4C5)cc3)cc21 ; CC1(C)c2cc(-c3ccccc3-c3ccccc3)ccc2-c2ccc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2cccc(-c3ccccc3-c3ccccc3)c21]

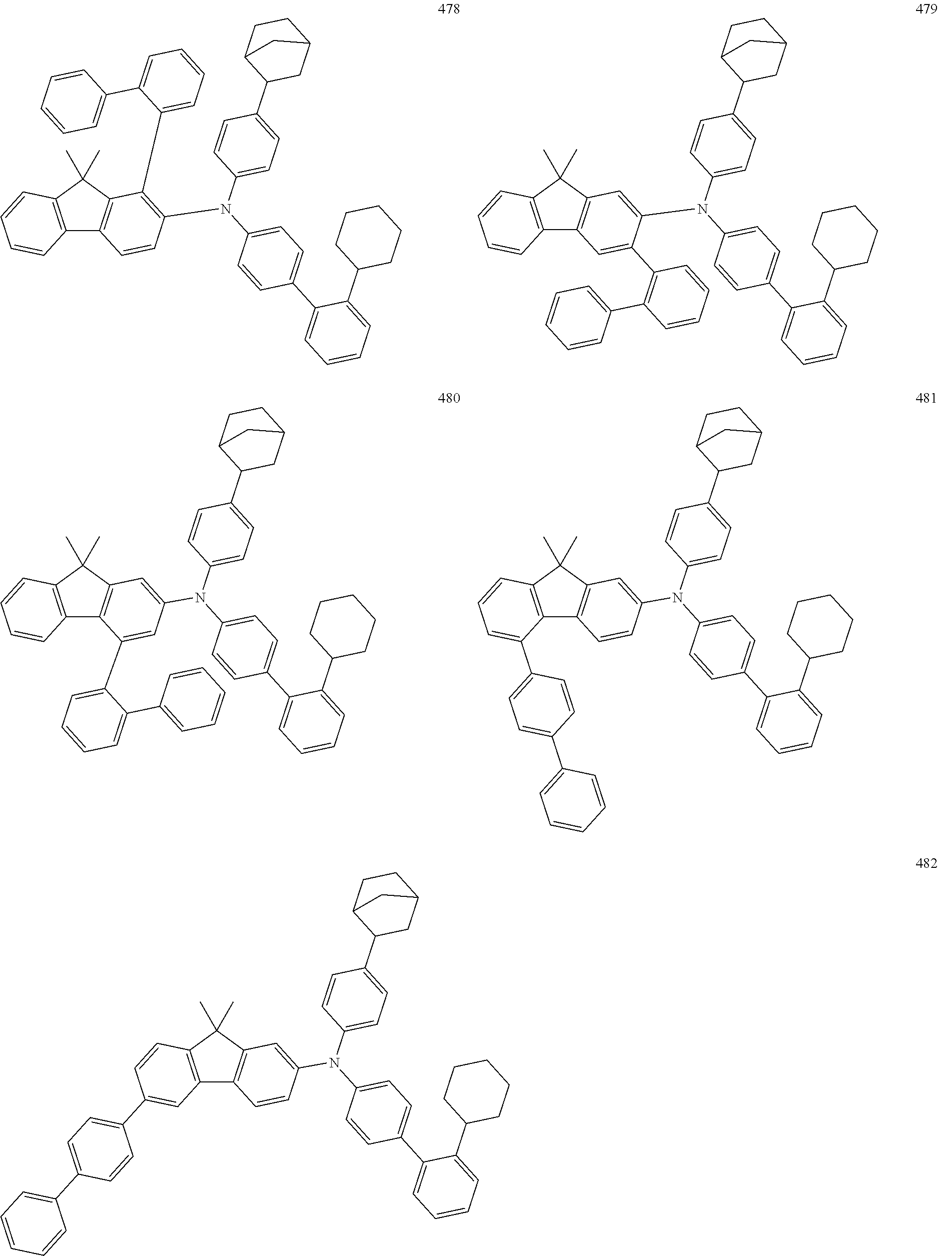

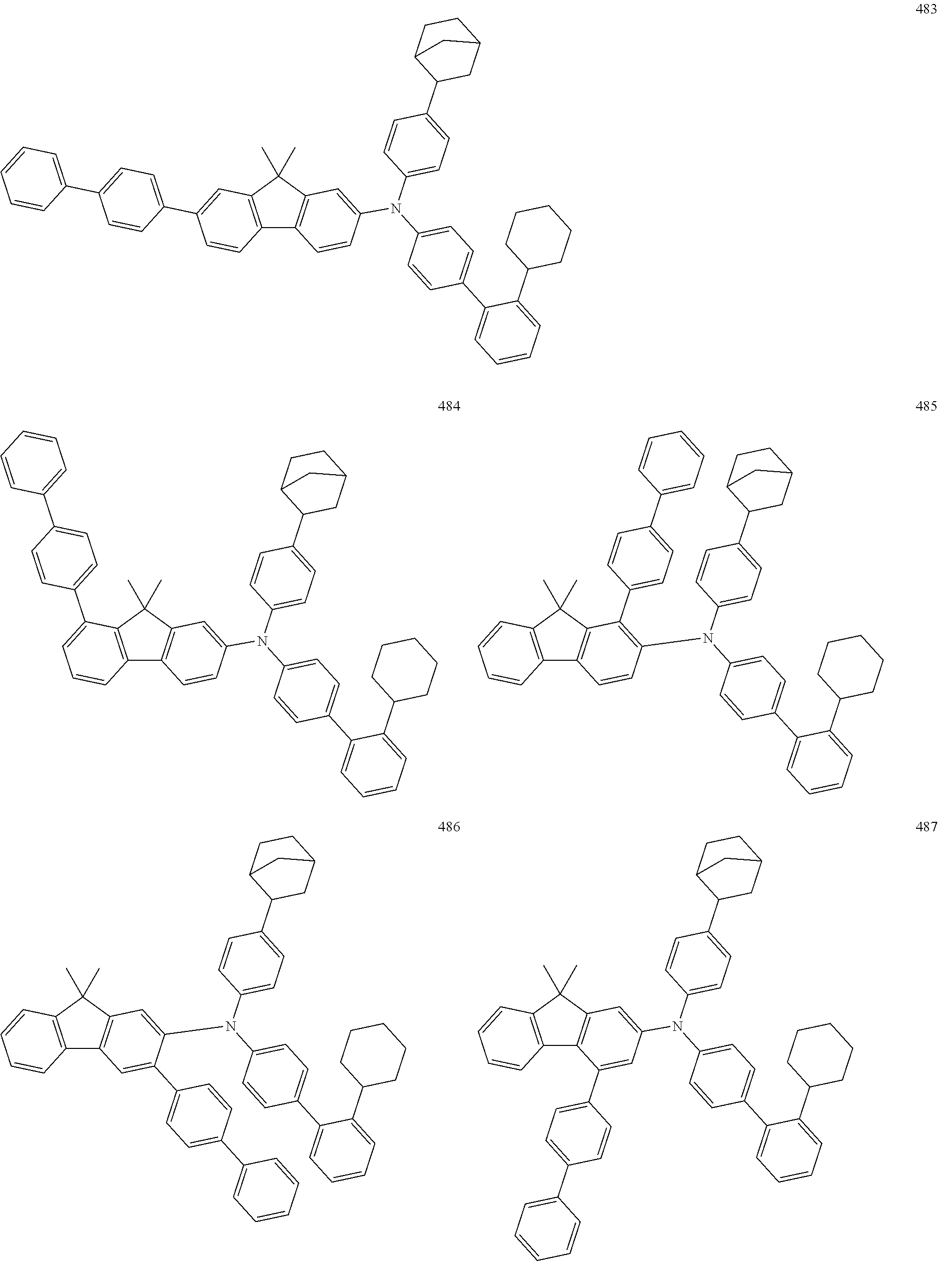

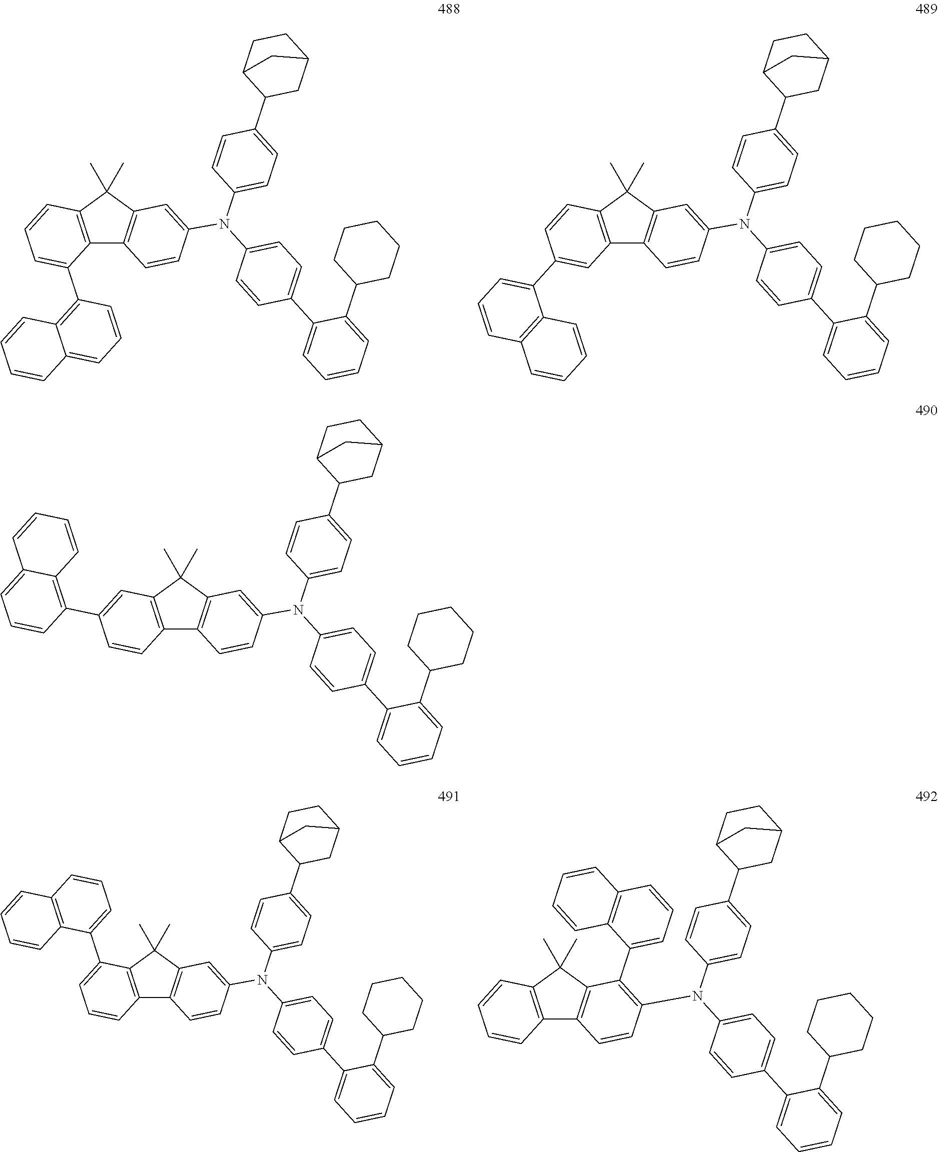 [SMILES: CC1(C)c2cc(N(c3ccc(-c4ccccc4C4CCCCC4)cc3)c3ccc(C4CC5CCC4C5)cc3)ccc2-c2c(-c3cccc4ccccc34)cccc21 ; CC1(C)c2ccc(-c3cccc4ccccc34)cc2-c2ccc(N(c3ccc(-c4ccccc4C4CCCCC4)cc3)c3ccc(C4CC5CCC4C5)cc3)cc21 ; CC1(C)c2cc(-c3cccc4ccccc34)ccc2-c2ccc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2cccc(-c3cccc4ccccc34)c21 ; CC1(C)c2ccccc2-c2ccc(N(c3ccc(-c4ccccc4C4CCCCC4)cc3)c3ccc(C4CC5CCC4C5)cc3)c(-c3cccc4ccccc34)c21]

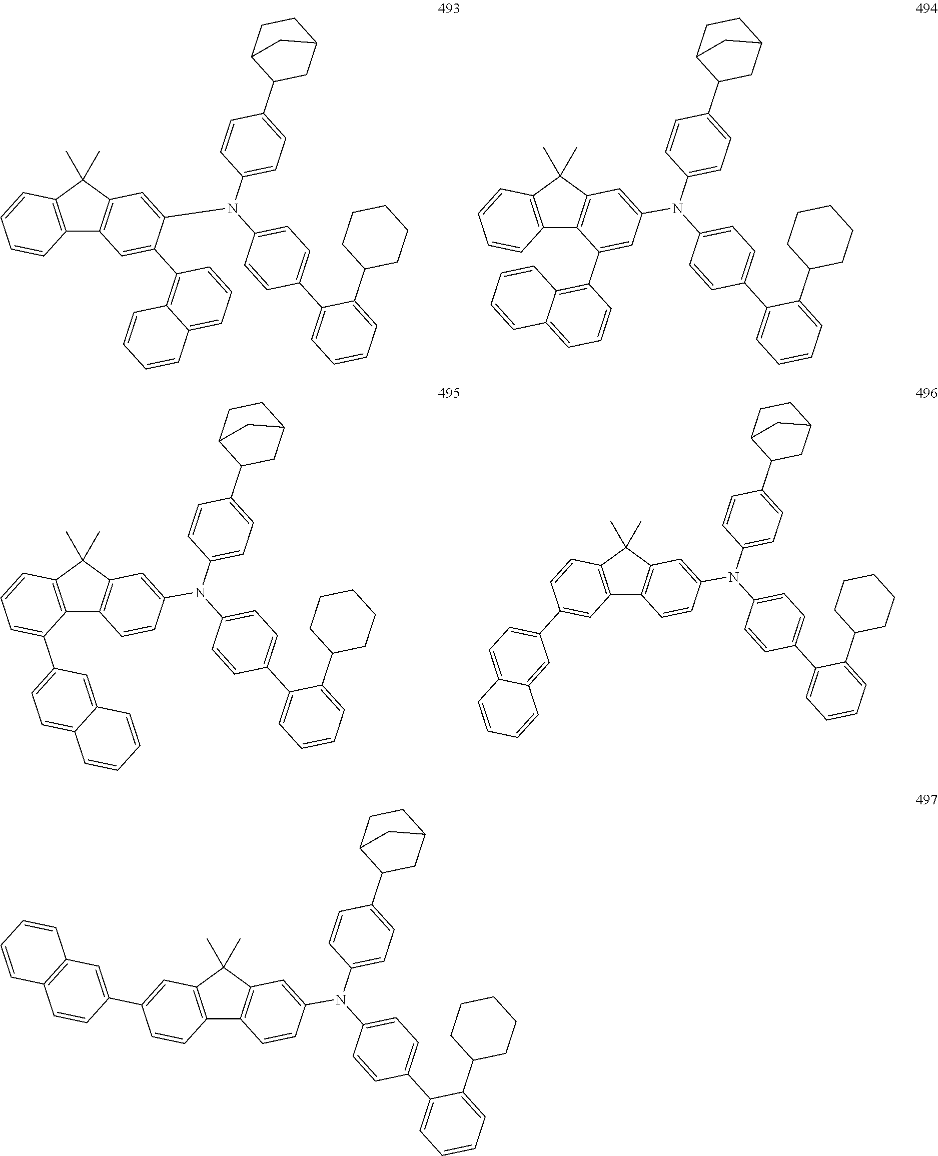

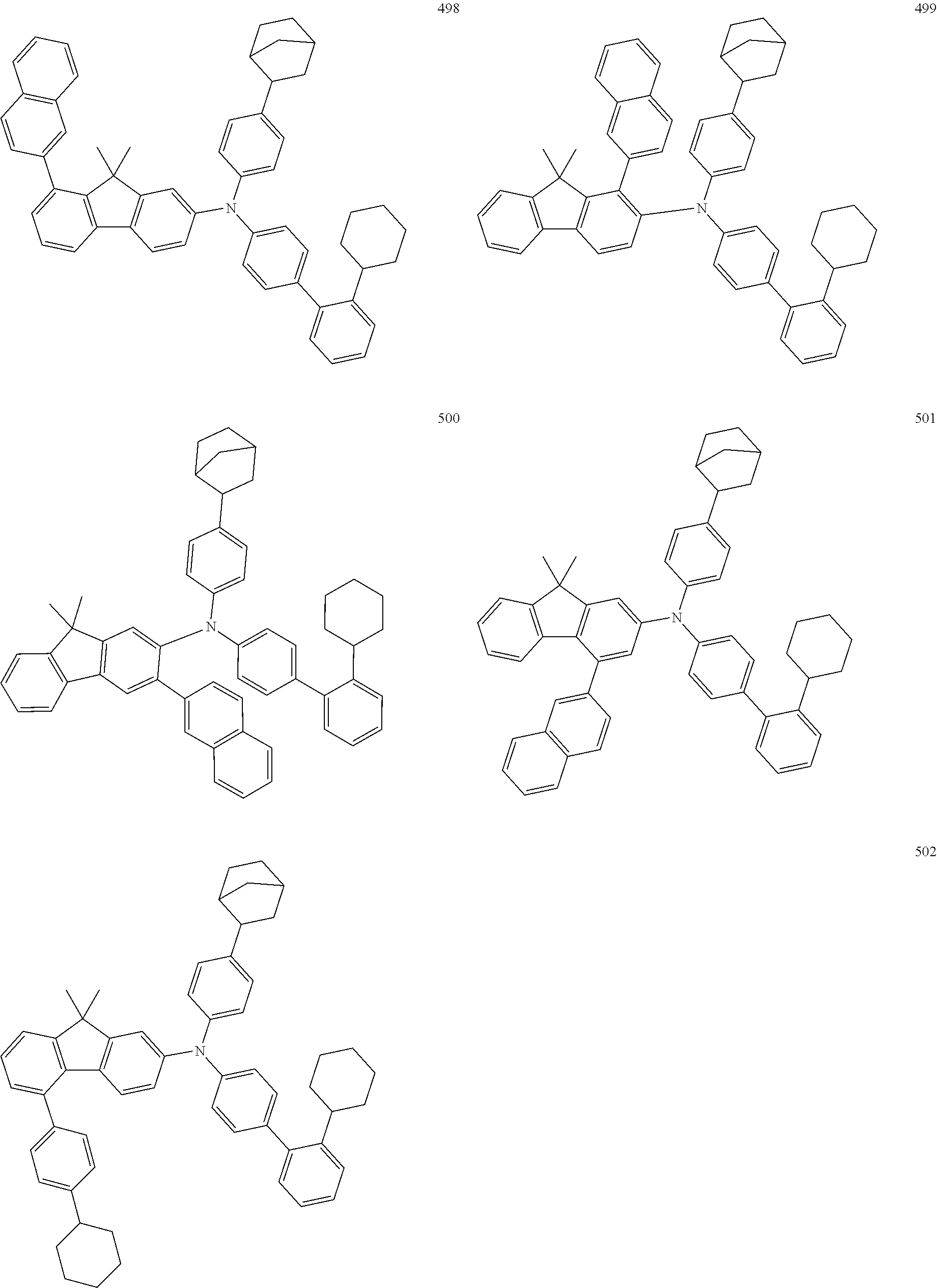 [SMILES: CC1(C)c2cc(N(c3ccc(-c4ccccc4C4CCCCC4)cc3)c3ccc(C4CC5CCC4C5)cc3)ccc2-c2cccc(-c3ccc4ccccc4c3)c21 ; CC1(C)c2ccccc2-c2ccc(N(c3ccc(-c4ccccc4C4CCCCC4)cc3)c3ccc(C4CC5CCC4C5)cc3)c(-c3ccc4ccccc4c3)c21 ; CC1(C)c2ccccc2-c2cc(-c3ccc4ccccc4c3)c(N(c3ccc(-c4ccccc4C4CCCCC4)cc3)c3ccc(C4CC5CCC4C5)cc3)cc21 ; CC1(C)c2ccccc2-c2c(-c3ccc4ccccc4c3)cc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2c(-c3ccc(C4CCCCC4)cc3)cccc21]

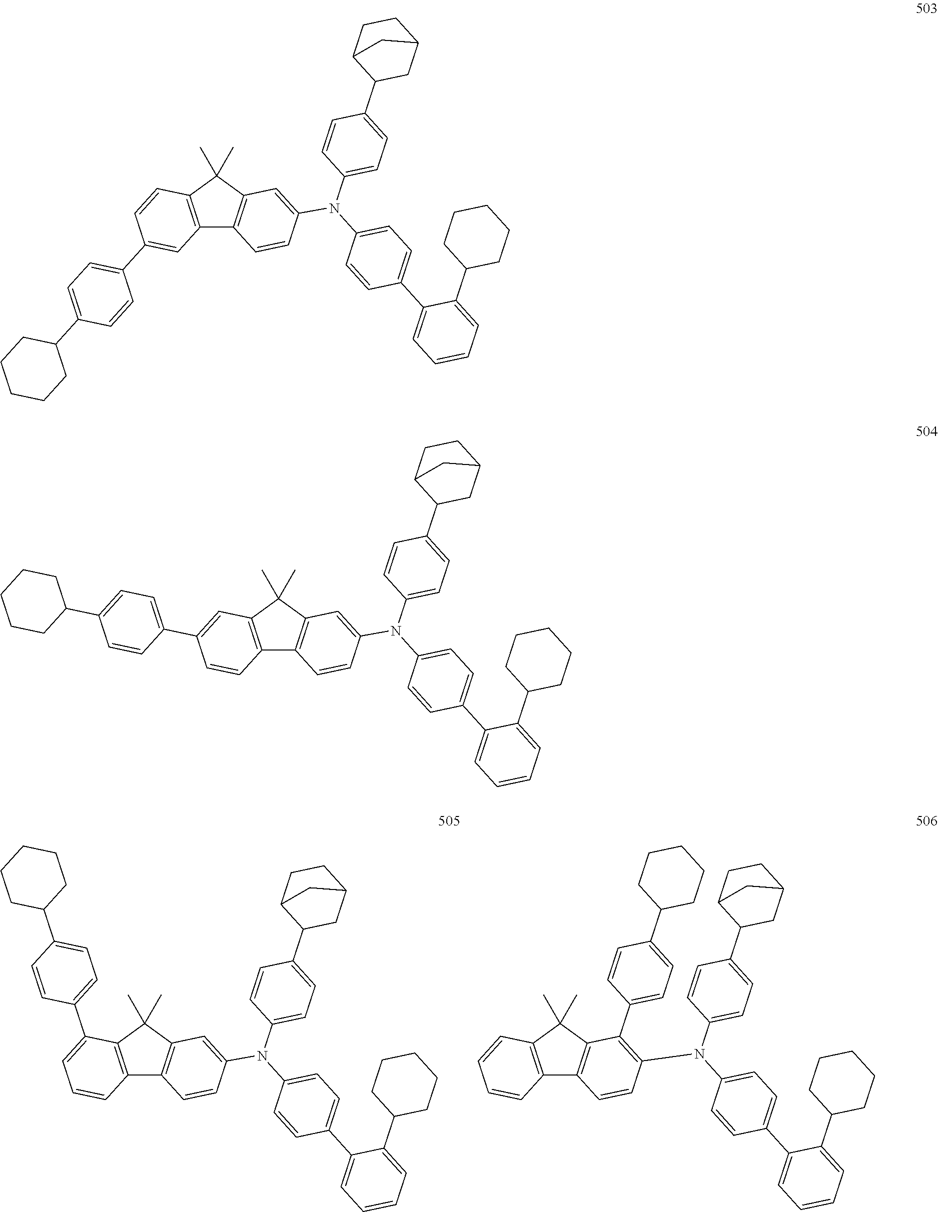

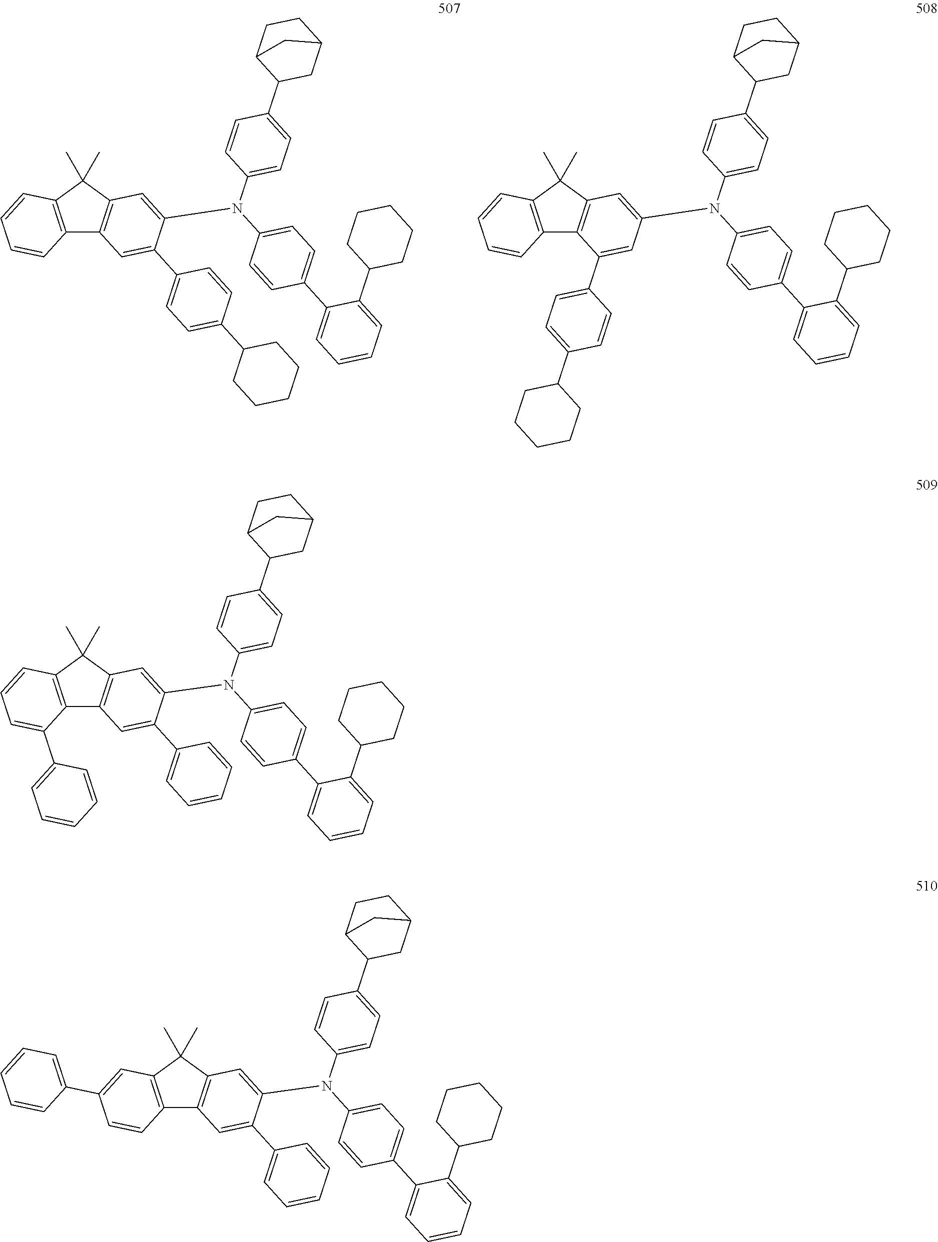 [SMILES: CC1(C)c2ccccc2-c2cc(-c3ccc(C4CCCCC4)cc3)c(N(c3ccc(-c4ccccc4C4CCCCC4)cc3)c3ccc(C4CC5CCC4C5)cc3)cc21 ; CC1(C)c2ccccc2-c2c(-c3ccc(C4CCCCC4)cc3)cc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)c(-c3ccccc3)cc2-c2c(-c3ccccc3)cccc21 ; CC1(C)c2cc(-c3ccccc3)ccc2-c2cc(-c3ccccc3)c(N(c3ccc(-c4ccccc4C4CCCCC4)cc3)c3ccc(C4CC5CCC4C5)cc3)cc21]

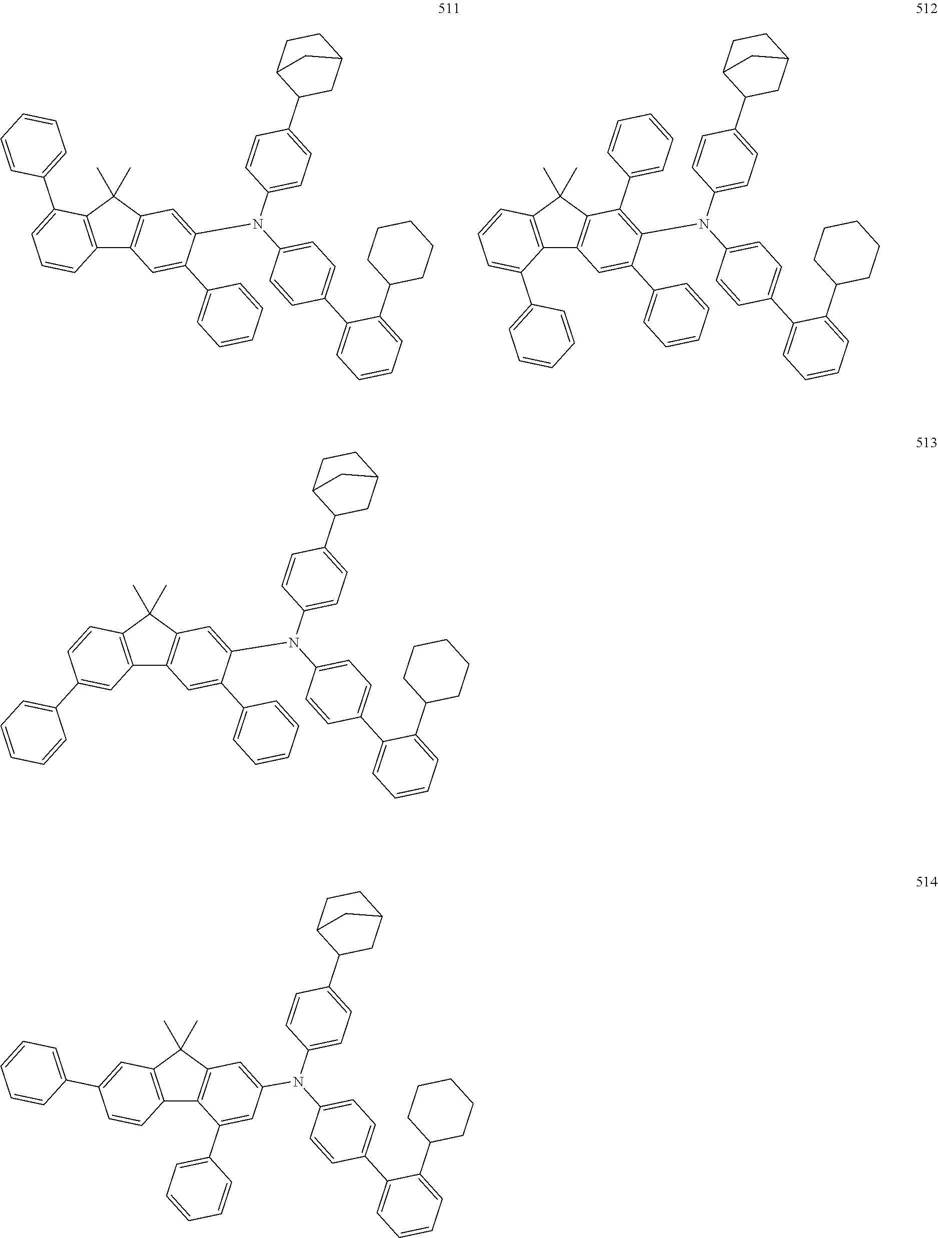 [SMILES: CC1(C)c2cc(N(c3ccc(-c4ccccc4C4CCCCC4)cc3)c3ccc(C4CC5CCC4C5)cc3)c(-c3ccccc3)cc2-c2cccc(-c3ccccc3)c21 ; CC1(C)c2cccc(-c3ccccc3)c2-c2cc(-c3ccccc3)c(N(c3ccc(-c4ccccc4C4CCCCC4)cc3)c3ccc(C4CC5CCC4C5)cc3)c(-c3ccccc3)c21 ; CC1(C)c2ccc(-c3ccccc3)cc2-c2cc(-c3ccccc3)c(N(c3ccc(-c4ccccc4C4CCCCC4)cc3)c3ccc(C4CC5CCC4C5)cc3)cc21 ; CC1(C)c2cc(-c3ccccc3)ccc2-c2c(-c3ccccc3)cc(N(c3ccc(-c4ccccc4C4CCCCC4)cc3)c3ccc(C4CC5CCC4C5)cc3)cc21]

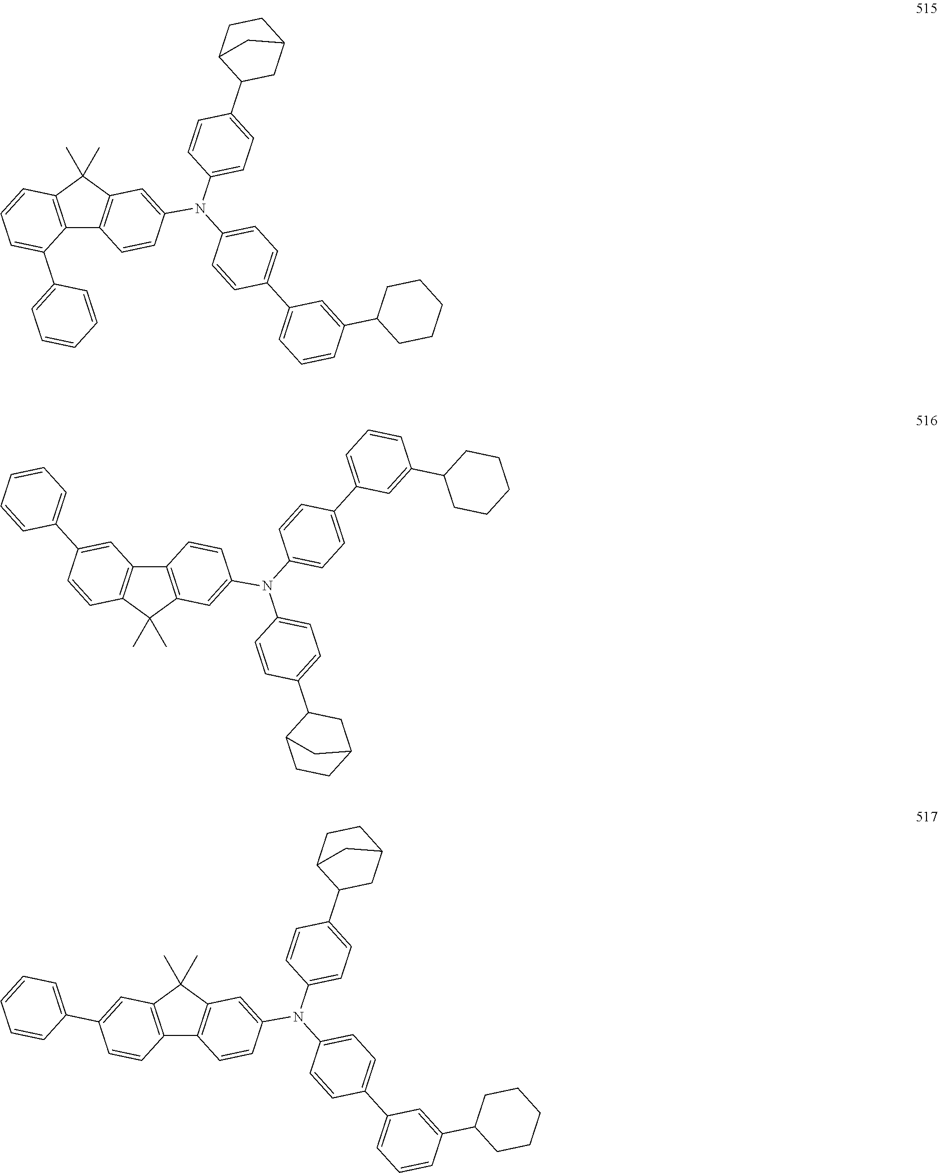 [SMILES: CC1(C)c2cc(N(c3ccc(-c4cccc(C5CCCCC5)c4)cc3)c3ccc(C4CC5CCC4C5)cc3)ccc2-c2c(-c3ccccc3)cccc21 ; CC1(C)c2ccc(-c3ccccc3)cc2-c2ccc(N(c3ccc(-c4cccc(C5CCCCC5)c4)cc3)c3ccc(C4CC5CCC4C5)cc3)cc21 ; CC1(C)c2cc(-c3ccccc3)ccc2-c2ccc(N(c3ccc(-c4cccc(C5CCCCC5)c4)cc3)c3ccc(C4CC5CCC4C5)cc3)cc21]

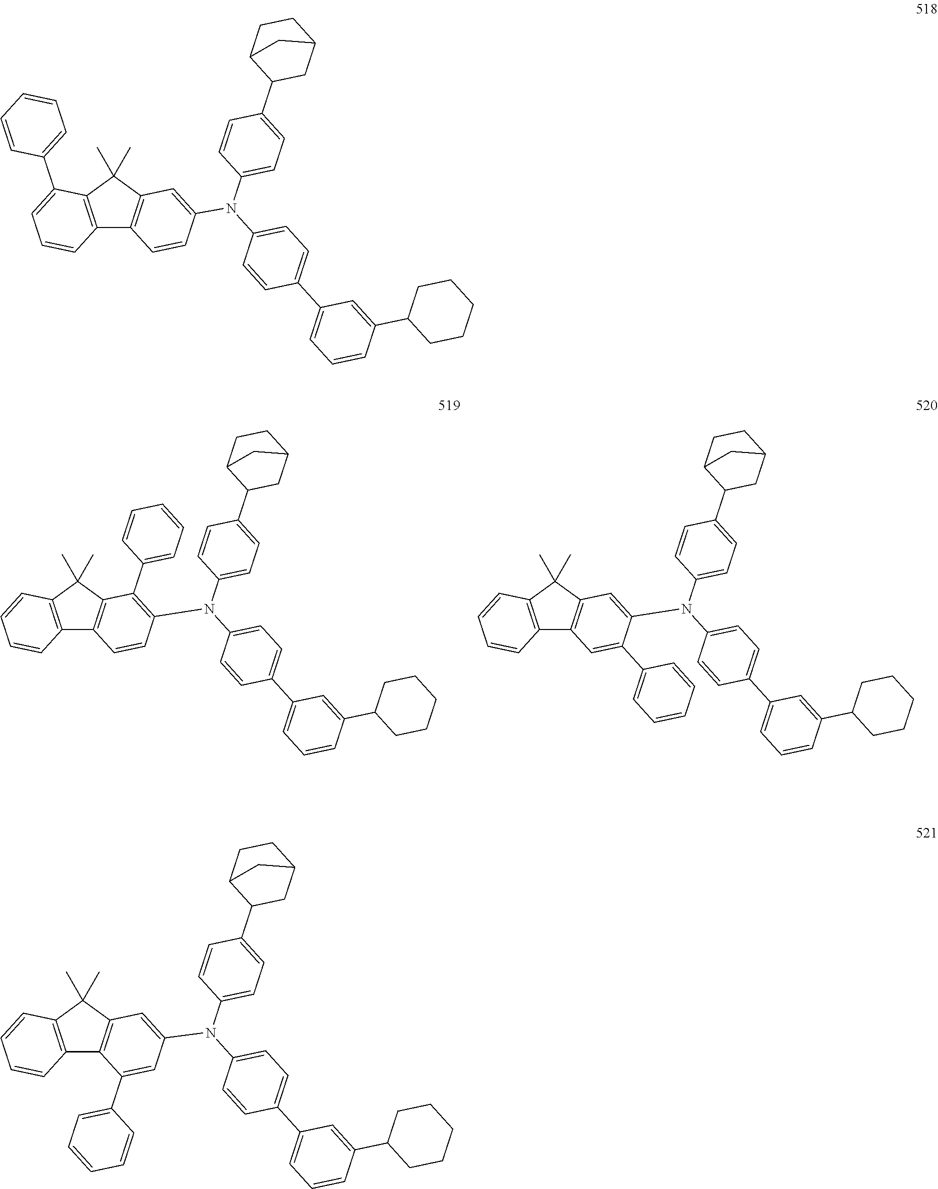 [SMILES: CC1(C)c2cc(N(c3ccc(-c4cccc(C5CCCCC5)c4)cc3)c3ccc(C4CC5CCC4C5)cc3)ccc2-c2cccc(-c3ccccc3)c21 ; CC1(C)c2ccccc2-c2ccc(N(c3ccc(-c4cccc(C5CCCCC5)c4)cc3)c3ccc(C4CC5CCC4C5)cc3)c(-c3ccccc3)c21 ; CC1(C)c2ccccc2-c2cc(-c3ccccc3)c(N(c3ccc(-c4cccc(C5CCCCC5)c4)cc3)c3ccc(C4CC5CCC4C5)cc3)cc21 ; CC1(C)c2ccccc2-c2c(-c3ccccc3)cc(N(c3ccc(-c4cccc(C5CCCCC5)c4)cc3)c3ccc(C4CC5CCC4C5)cc3)cc21]

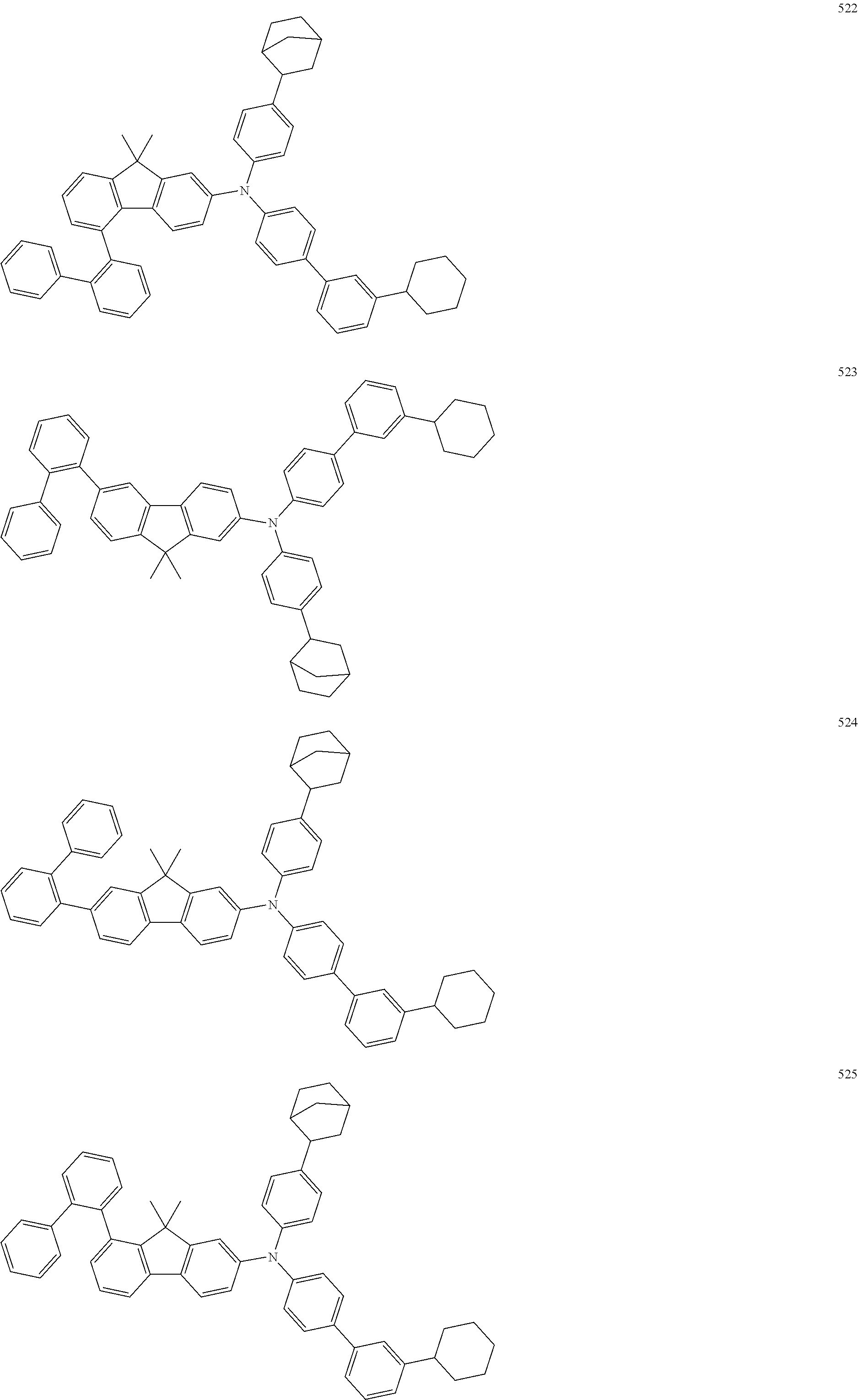

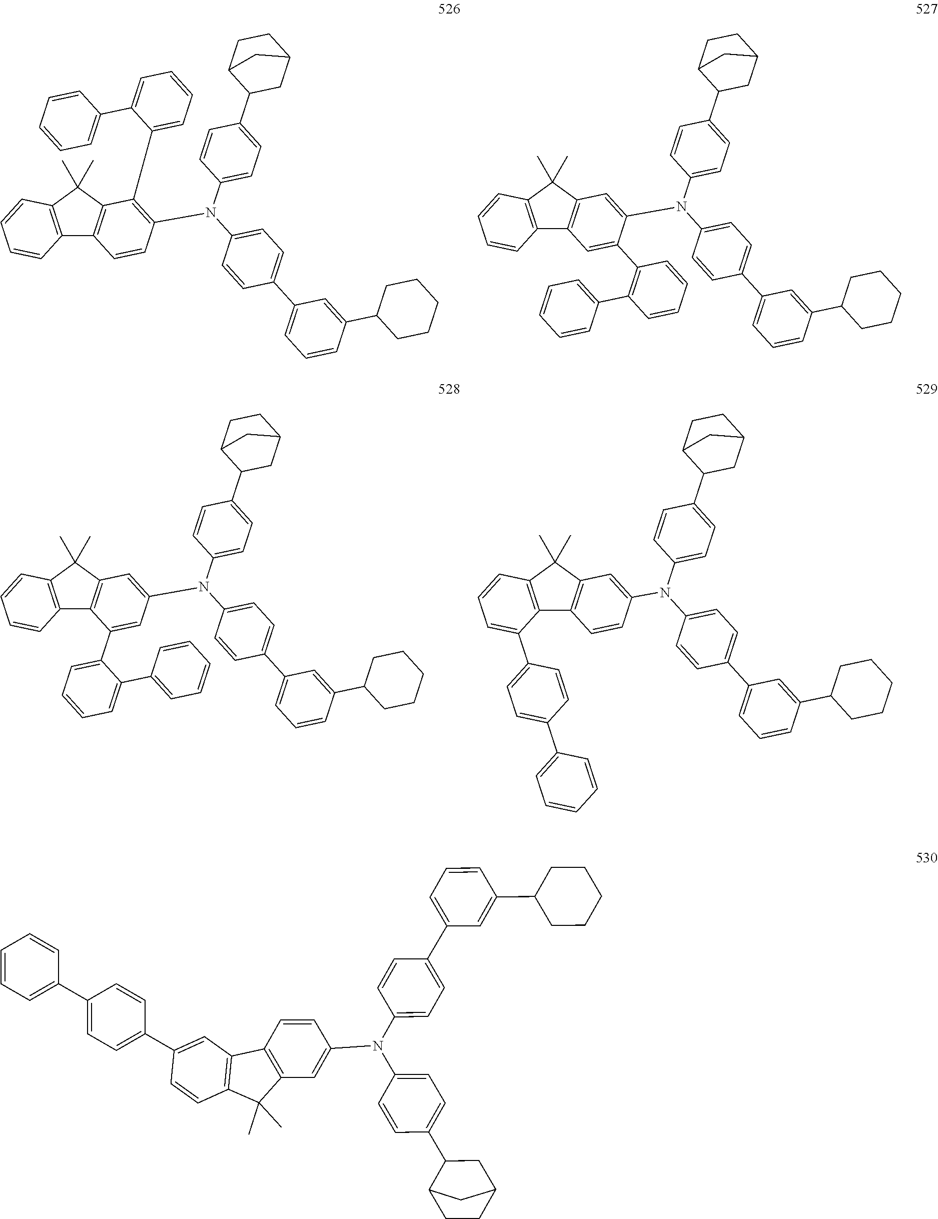

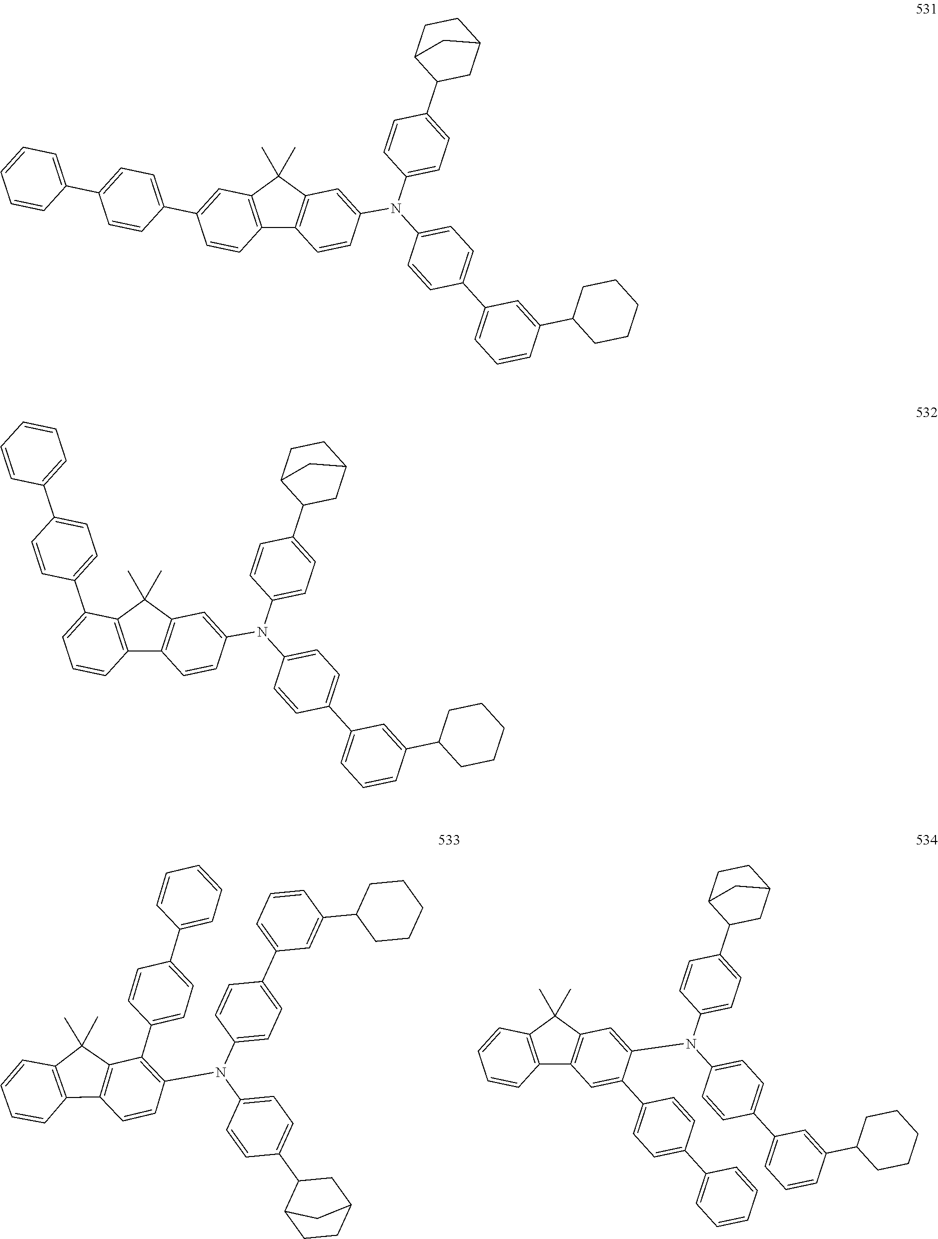 [SMILES: CC1(C)c2cc(-c3ccc(-c4ccccc4)cc3)ccc2-c2ccc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2cccc(-c3ccc(-c4ccccc4)cc3)c21 ; CC1(C)c2ccccc2-c2ccc(N(c3ccc(-c4cccc(C5CCCCC5)c4)cc3)c3ccc(C4CC5CCC4C5)cc3)c(-c3ccc(-c4ccccc4)cc3)c21 ; CC1(C)c2ccccc2-c2cc(-c3ccc(-c4ccccc4)cc3)c(N(c3ccc(-c4cccc(C5CCCCC5)c4)cc3)c3ccc(C4CC5CCC4C5)cc3)cc21]

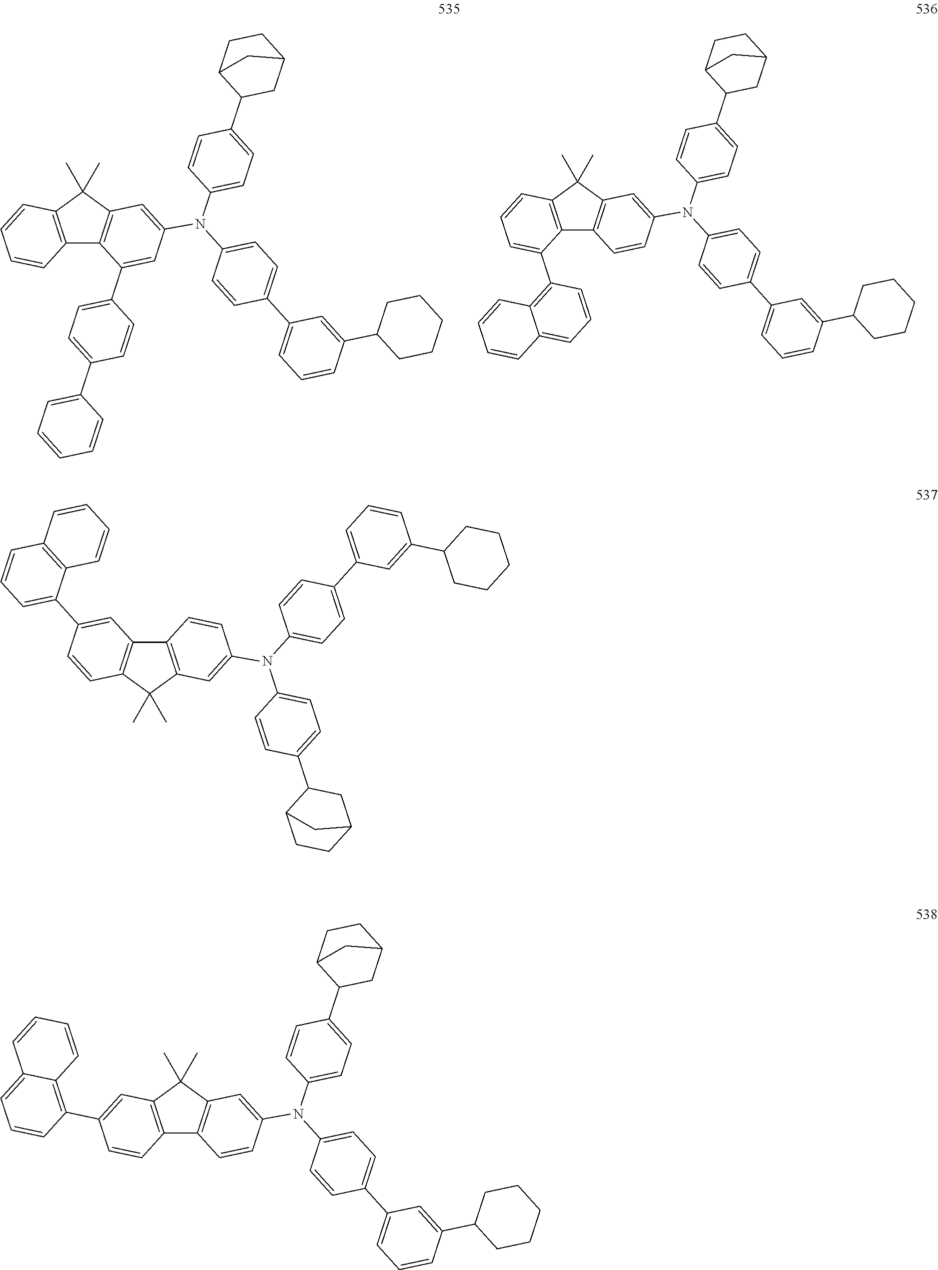

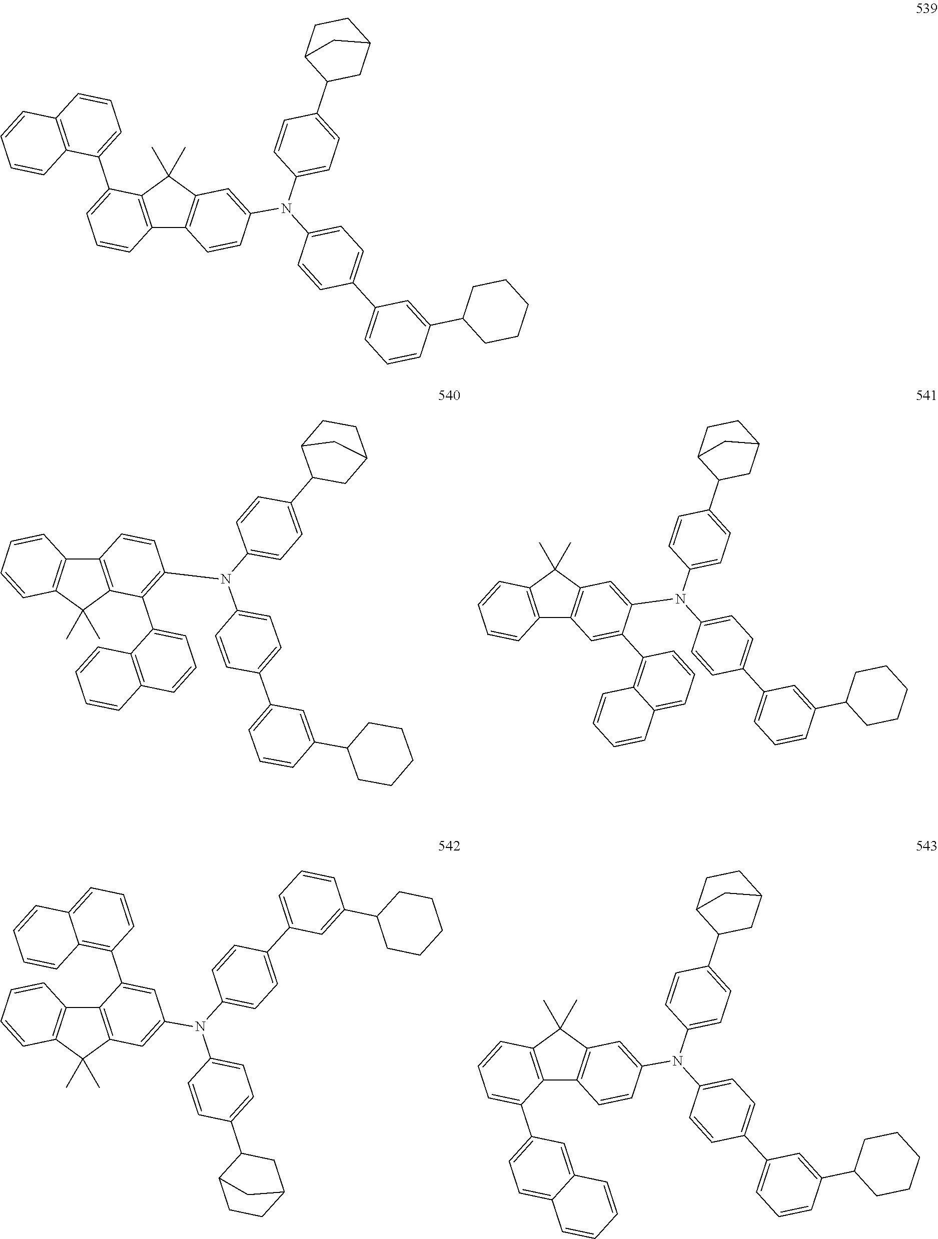 [SMILES: CC1(C)c2cc(N(c3ccc(-c4cccc(C5CCCCC5)c4)cc3)c3ccc(C4CC5CCC4C5)cc3)ccc2-c2cccc(-c3cccc4ccccc34)c21 ; CC1(C)c2ccccc2-c2ccc(N(c3ccc(-c4cccc(C5CCCCC5)c4)cc3)c3ccc(C4CC5CCC4C5)cc3)c(-c3cccc4ccccc34)c21 ; CC1(C)c2ccccc2-c2cc(-c3cccc4ccccc34)c(N(c3ccc(-c4cccc(C5CCCCC5)c4)cc3)c3ccc(C4CC5CCC4C5)cc3)cc21 ; CC1(C)c2ccccc2-c2c(-c3cccc4ccccc34)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2c(-c3ccc4ccccc4c3)cccc21]

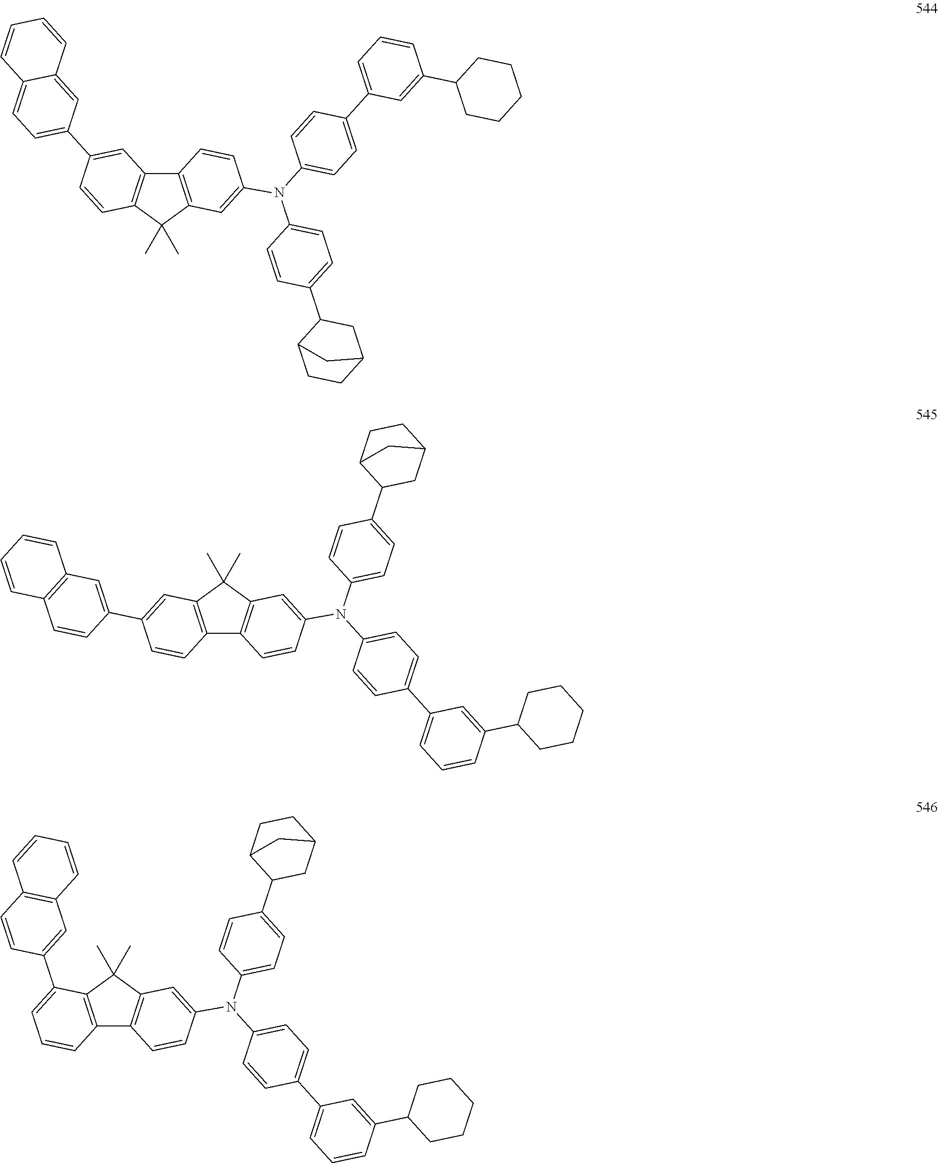 [SMILES: CC1(C)c2ccc(-c3ccc4ccccc4c3)cc2-c2ccc(N(c3ccc(-c4cccc(C5CCCCC5)c4)cc3)c3ccc(C4CC5CCC4C5)cc3)cc21 ; CC1(C)c2cc(-c3ccc4ccccc4c3)ccc2-c2ccc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2cccc(-c3ccc4ccccc4c3)c21]

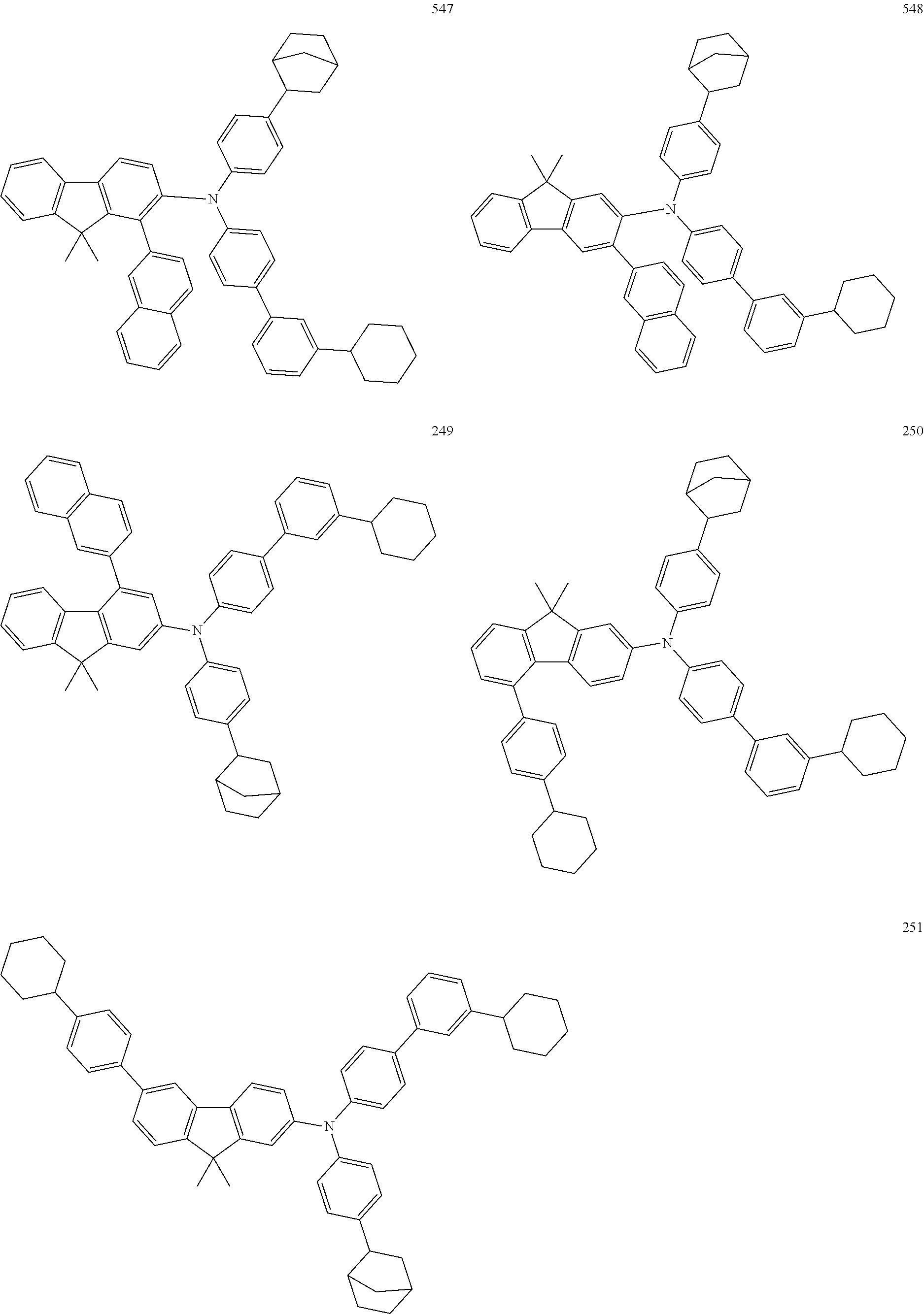 [SMILES: CC1(C)c2ccccc2-c2ccc(N(c3ccc(-c4cccc(C5CCCCC5)c4)cc3)c3ccc(C4CC5CCC4C5)cc3)c(-c3ccc4ccccc4c3)c21 ; CC1(C)c2ccccc2-c2cc(-c3ccc4ccccc4c3)c(N(c3ccc(-c4cccc(C5CCCCC5)c4)cc3)c3ccc(C4CC5CCC4C5)cc3)cc21 ; CC1(C)c2ccccc2-c2c(-c3ccc4ccccc4c3)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(-c4cccc(C5CCCCC5)c4)cc3)c3ccc(C4CC5CCC4C5)cc3)cc21]

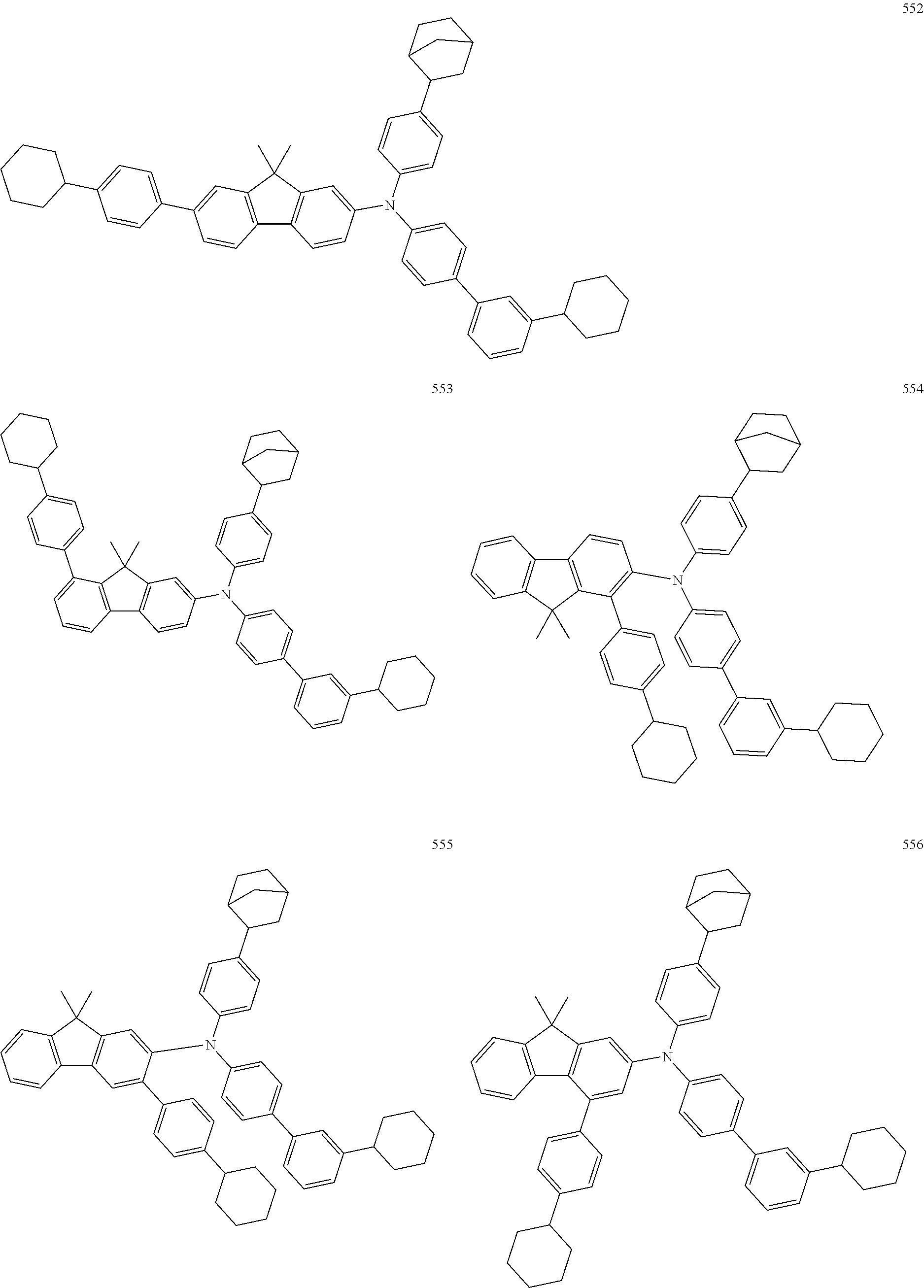

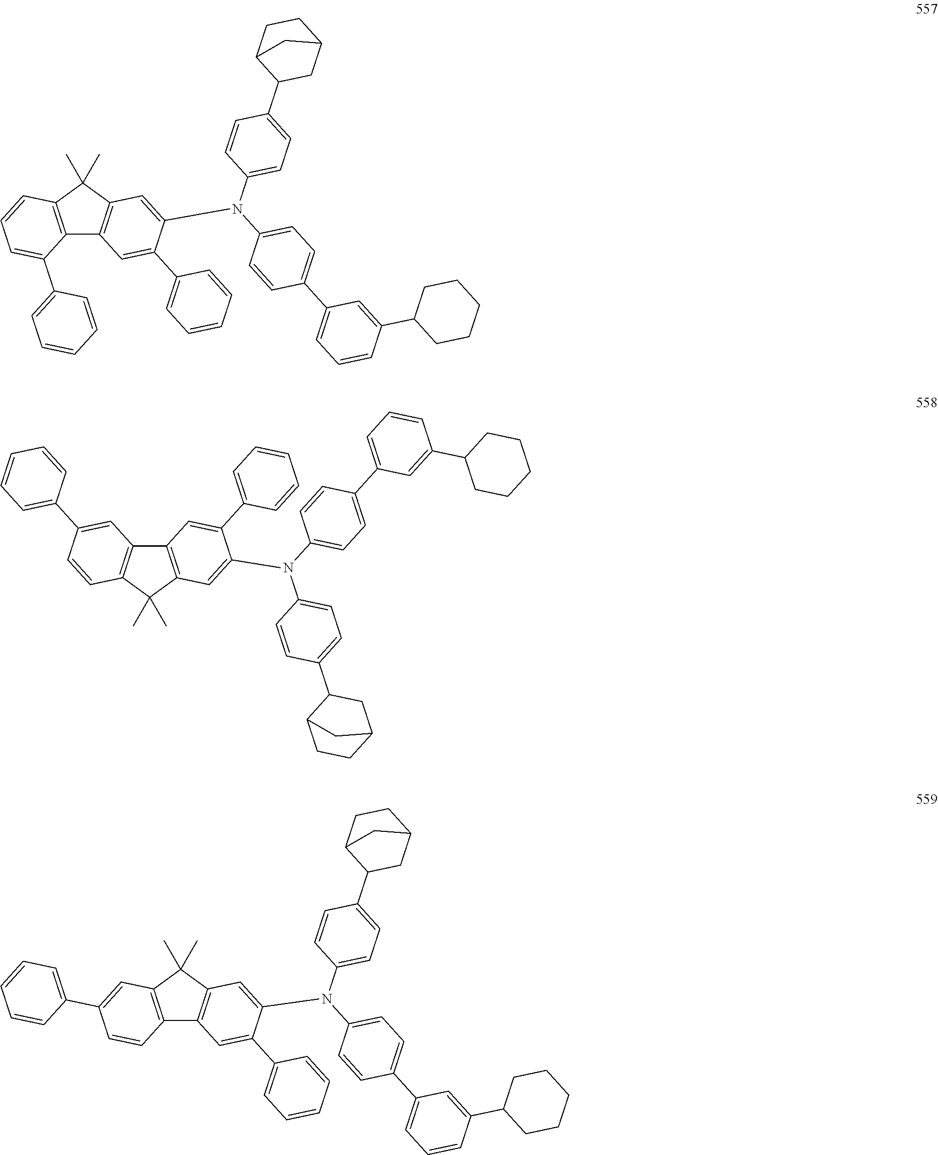 [SMILES: CC1(C)c2cc(N(c3ccc(-c4cccc(C5CCCCC5)c4)cc3)c3ccc(C4CC5CCC4C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(-c4cccc(C5CCCCC5)c4)cc3)c3ccc(C4CC5CCC4C5)cc3)cc21 ; CC1(C)c2cc(-c3ccccc3)ccc2-c2cc(-c3ccccc3)c(N(c3ccc(-c4cccc(C5CCCCC5)c4)cc3)c3ccc(C4CC5CCC4C5)cc3)cc21]

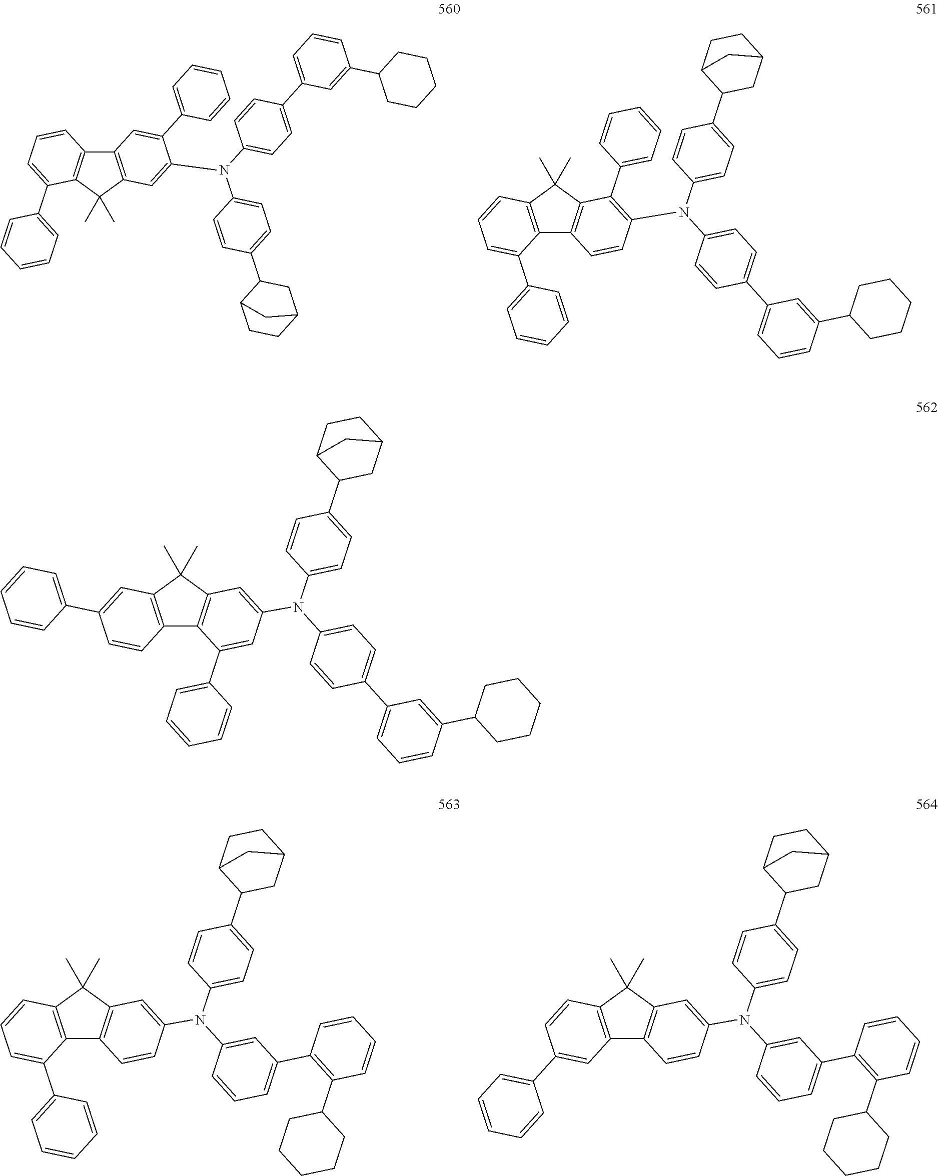 [SMILES: CC1(C)c2cc(N(c3ccc(-c4cccc(C5CCCCC5)c4)cc3)c3ccc(C4CC5CCC4C5)cc3)c(-c3ccccc3)cc2-c2cccc(-c3ccccc3)c21 ; CC1(C)c2cccc(-c3ccccc3)c2-c2ccc(N(c3ccc(-c4cccc(C5CCCCC5)c4)cc3)c3ccc(C4CC5CCC4C5)cc3)c(-c3ccccc3)c21 ; CC1(C)c2cc(-c3ccccc3)ccc2-c2c(-c3ccccc3)cc(N(c3ccc(-c4cccc(C5CCCCC5)c4)cc3)c3ccc(C4CC5CCC4C5)cc3)cc21 ; CC1(C)c2cc(N(c3ccc(C4CC5CCC4C5)cc3)c3cccc(-c4ccccc4C4CCCCC4)c3)ccc2-c2c(-c3ccccc3)cccc21 ; CC1(C)c2ccc(-c3ccccc3)cc2-c2ccc(N(c3ccc(C4CC5CCC4C5)cc3)c3cccc(-c4ccccc4C4CCCCC4)c3)cc21]

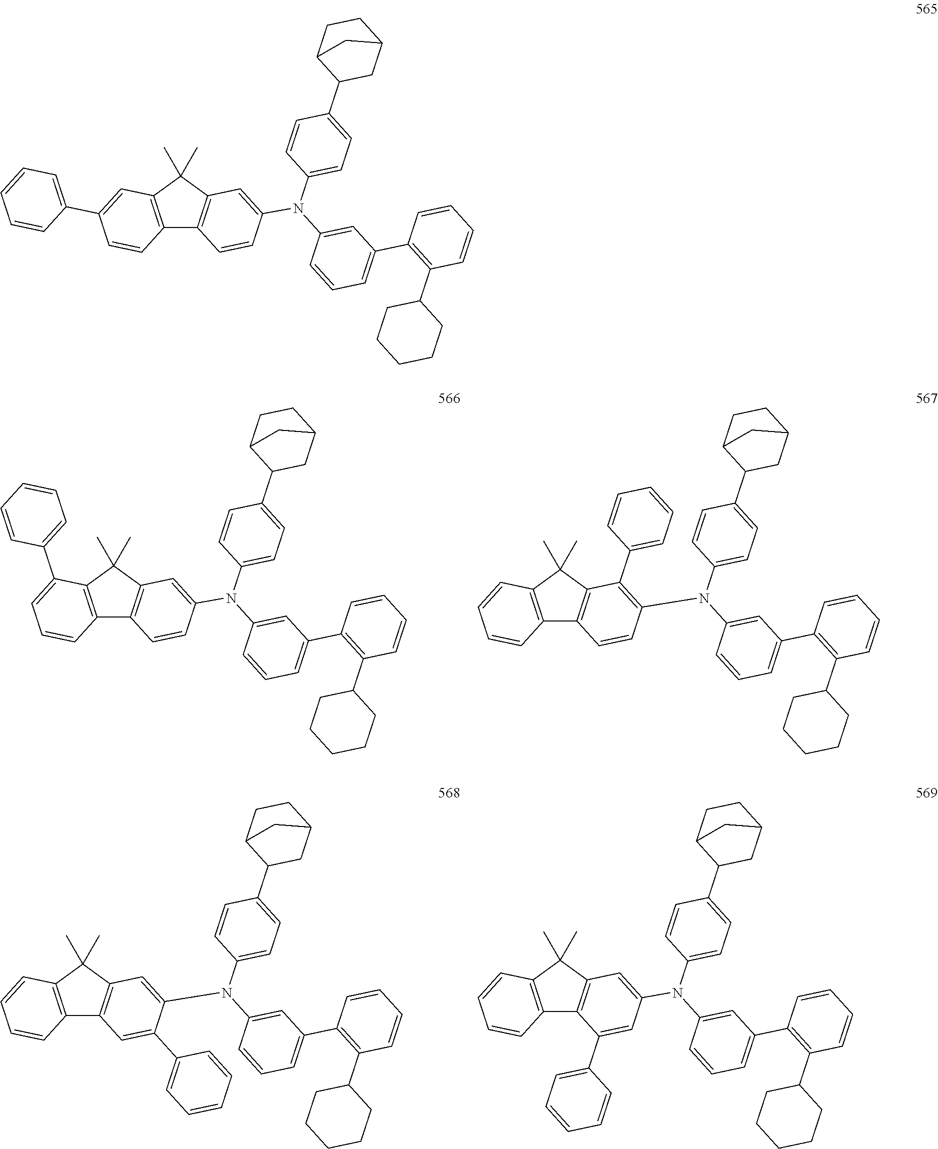 [SMILES: CC1(C)c2cc(-c3ccccc3)ccc2-c2ccc(N(c3ccc(C4CC5CCC4C5)cc3)c3cccc(-c4ccccc4C4CCCCC4)c3)cc21 ; CC1(C)c2cc(N(c3ccc(C4CC5CCC4C5)cc3)c3cccc(-c4ccccc4C4CCCCC4)c3)ccc2-c2cccc(-c3ccccc3)c21 ; CC1(C)c2ccccc2-c2ccc(N(c3ccc(C4CC5CCC4C5)cc3)c3cccc(-c4ccccc4C4CCCCC4)c3)c(-c3ccccc3)c21 ; CC1(C)c2ccccc2-c2cc(-c3ccccc3)c(N(c3ccc(C4CC5CCC4C5)cc3)c3cccc(-c4ccccc4C4CCCCC4)c3)cc21 ; CC1(C)c2ccccc2-c2c(-c3ccccc3)cc(N(c3ccc(C4CC5CCC4C5)cc3)c3cccc(-c4ccccc4C4CCCCC4)c3)cc21]

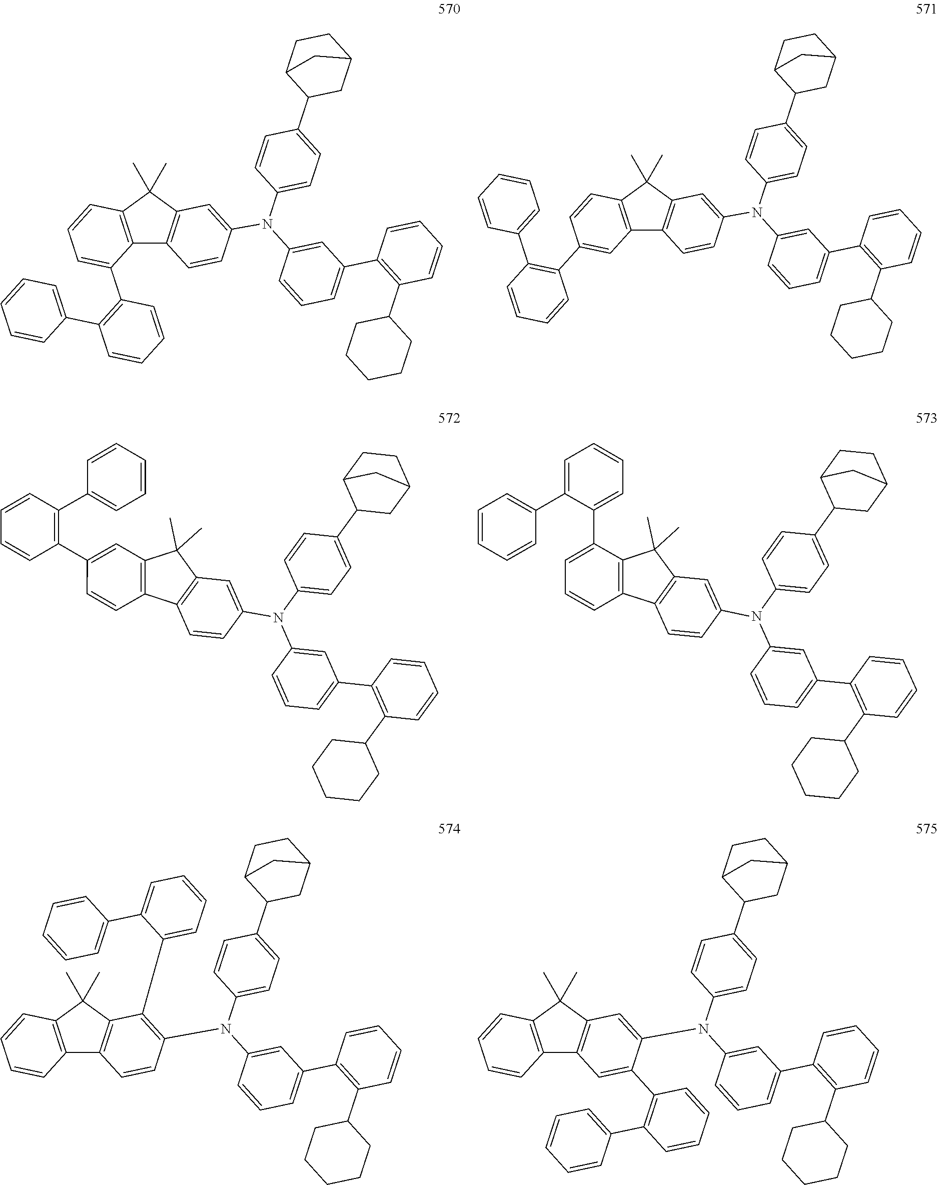

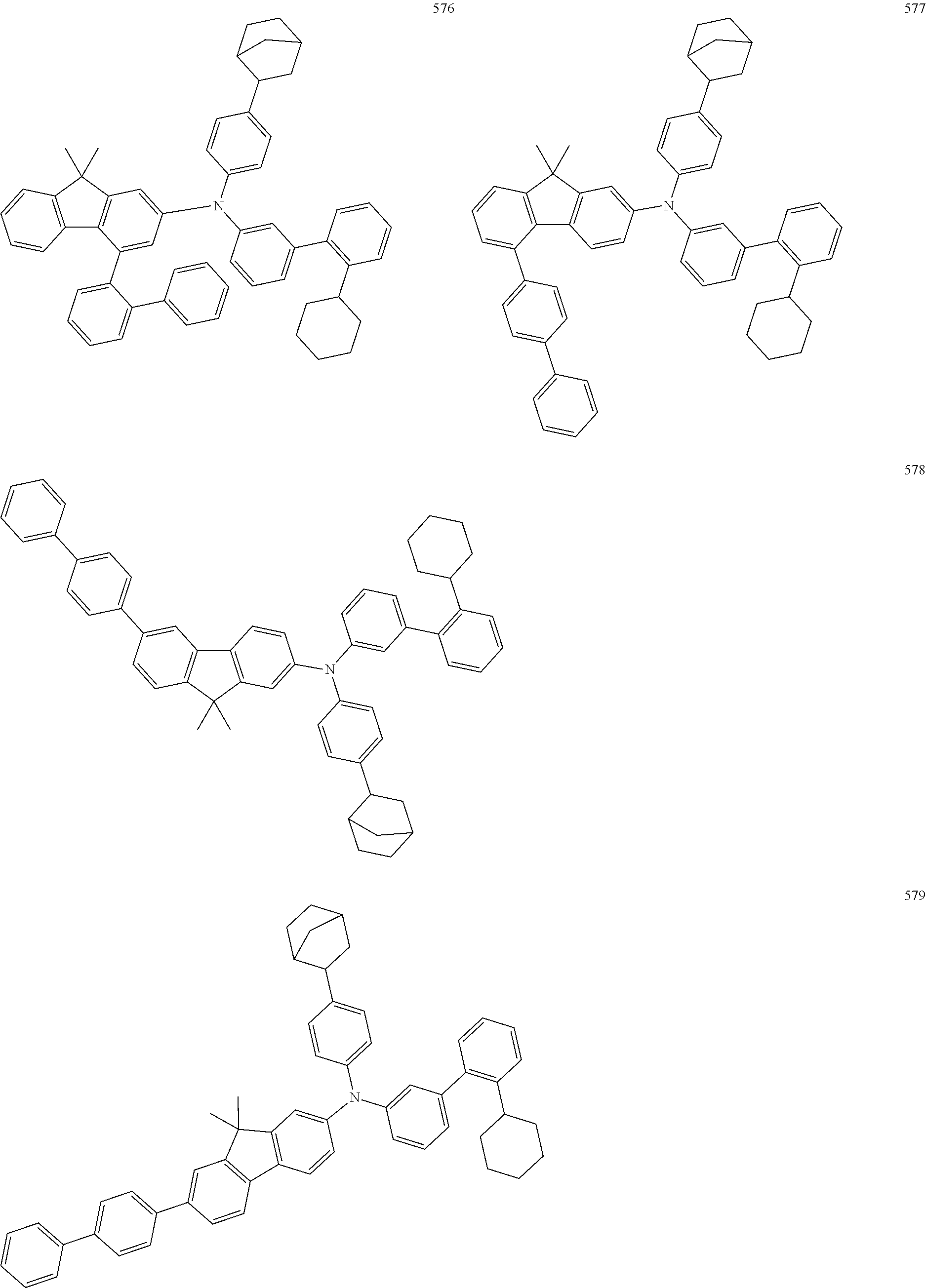 [SMILES: CC1(C)c2ccccc2-c2c(-c3ccccc3-c3ccccc3)cc(N(c3ccc(C4CC5CCC4C5)cc3)c3cccc(-c4ccccc4C4CCCCC4)c3)cc21 ; CC1(C)c2cc(N(c3ccc(C4CC5CCC4C5)cc3)c3cccc(-c4ccccc4C4CCCCC4)c3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CC5CCC4C5)cc3)c3cccc(-c4ccccc4C4CCCCC4)c3)cc21 ; CC1(C)c2cc(-c3ccc(-c4ccccc4)cc3)ccc2-c2ccc(N(c3ccc(C4CC5CCC4C5)cc3)c3cccc(-c4ccccc4C4CCCCC4)c3)cc21]

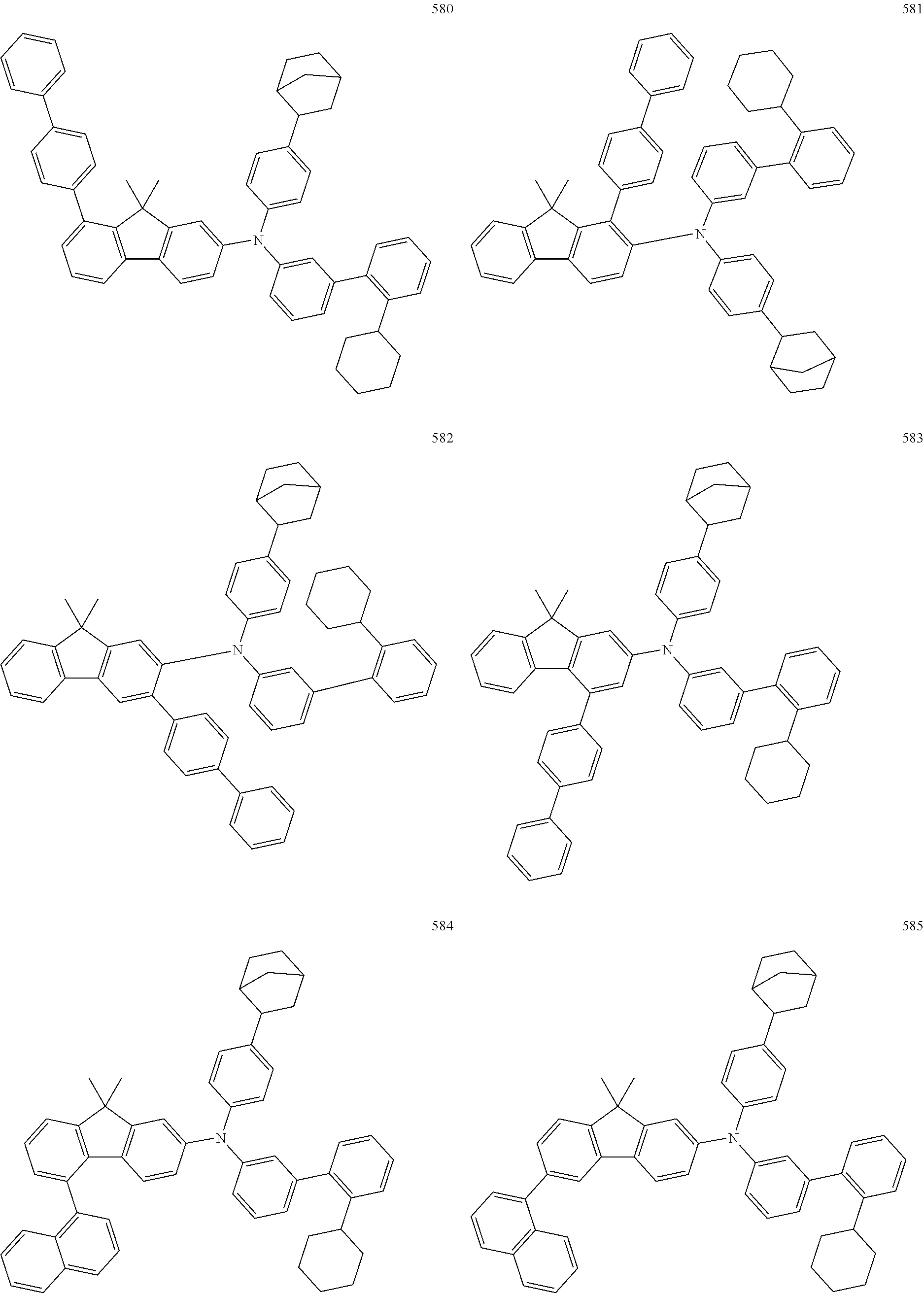

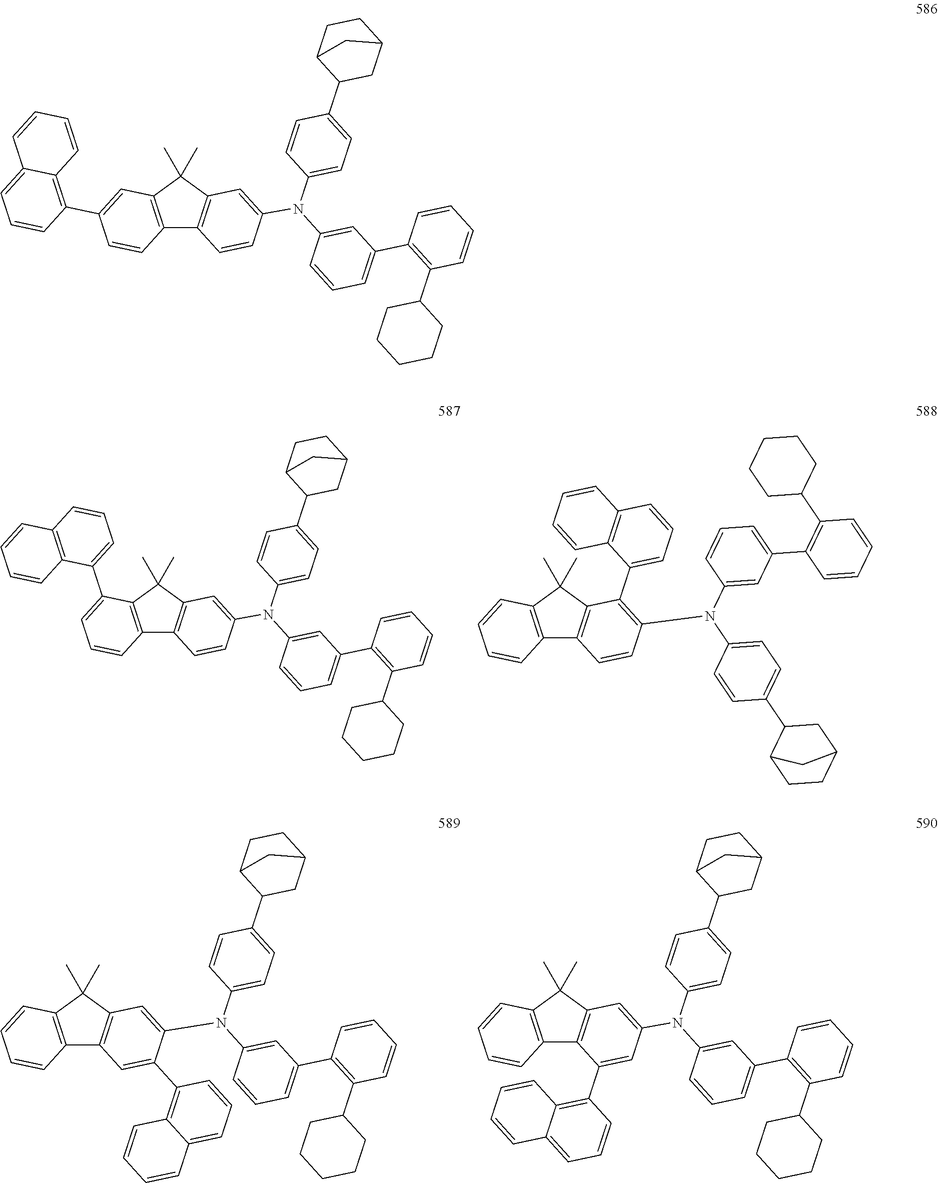 [SMILES: CC1(C)c2cc(-c3cccc4ccccc34)ccc2-c2ccc(N(c3ccc(C4CC5CCC4C5)cc3)c3cccc(-c4ccccc4C4CCCCC4)c3)cc21 ; CC1(C)c2cc(N(c3ccc(C4CC5CCC4C5)cc3)c3cccc(-c4ccccc4C4CCCCC4)c3)ccc2-c2cccc(-c3cccc4ccccc34)c21 ; CC1(C)c2ccccc2-c2ccc(N(c3ccc(C4CC5CCC4C5)cc3)c3cccc(-c4ccccc4C4CCCCC4)c3)c(-c3cccc4ccccc34)c21 ; CC1(C)c2ccccc2-c2cc(-c3cccc4ccccc34)c(N(c3ccc(C4CC5CCC4C5)cc3)c3cccc(-c4ccccc4C4CCCCC4)c3)cc21 ; CC1(C)c2ccccc2-c2c(-c3cccc4ccccc34)cc(N(c3ccc(C4CC5CCC4C5)cc3)c3cccc(-c4ccccc4C4CCCCC4)c3)cc21]

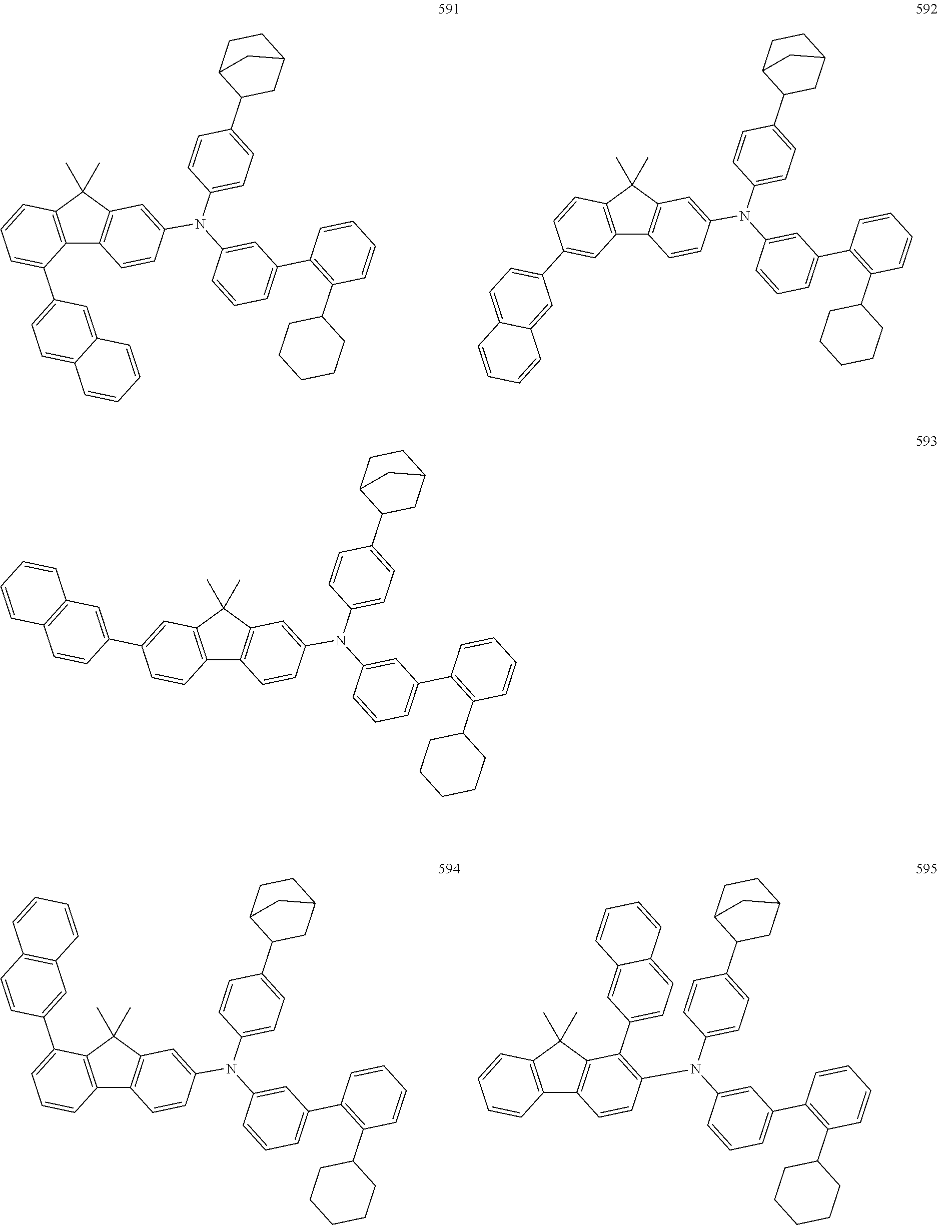 [SMILES: CC1(C)c2cc(N(c3ccc(C4CC5CCC4C5)cc3)c3cccc(-c4ccccc4C4CCCCC4)c3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3cccc(-c4ccccc4C4CCCCC4)c3)cc21 ; CC1(C)c2cc(-c3ccc4ccccc4c3)ccc2-c2ccc(N(c3ccc(C4CC5CCC4C5)cc3)c3cccc(-c4ccccc4C4CCCCC4)c3)cc21 ; CC1(C)c2cc(N(c3ccc(C4CC5CCC4C5)cc3)c3cccc(-c4ccccc4C4CCCCC4)c3)ccc2-c2cccc(-c3ccc4ccccc4c3)c21 ; CC1(C)c2ccccc2-c2ccc(N(c3ccc(C4CC5CCC4C5)cc3)c3cccc(-c4ccccc4C4CCCCC4)c3)c(-c3ccc4ccccc4c3)c21]

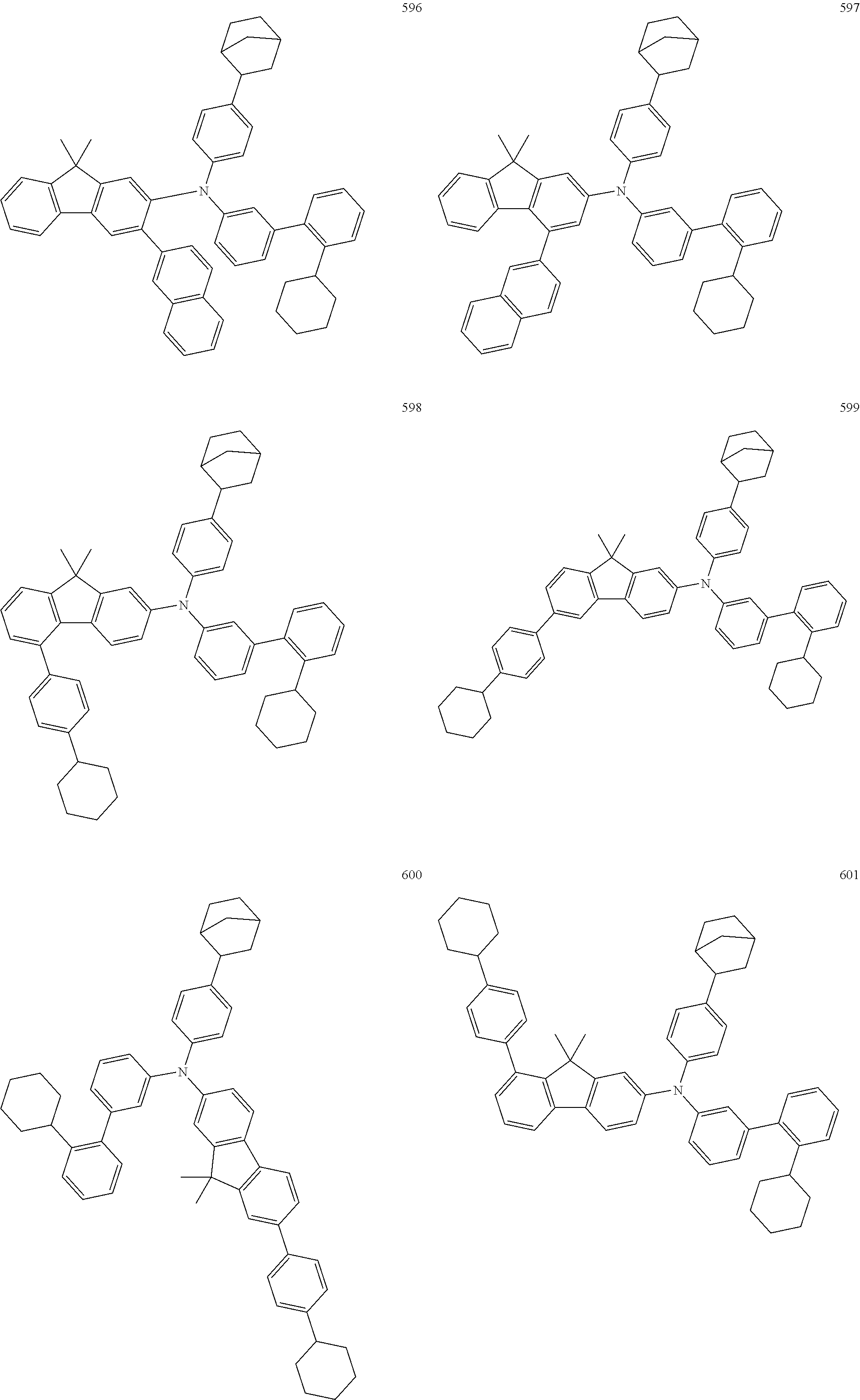

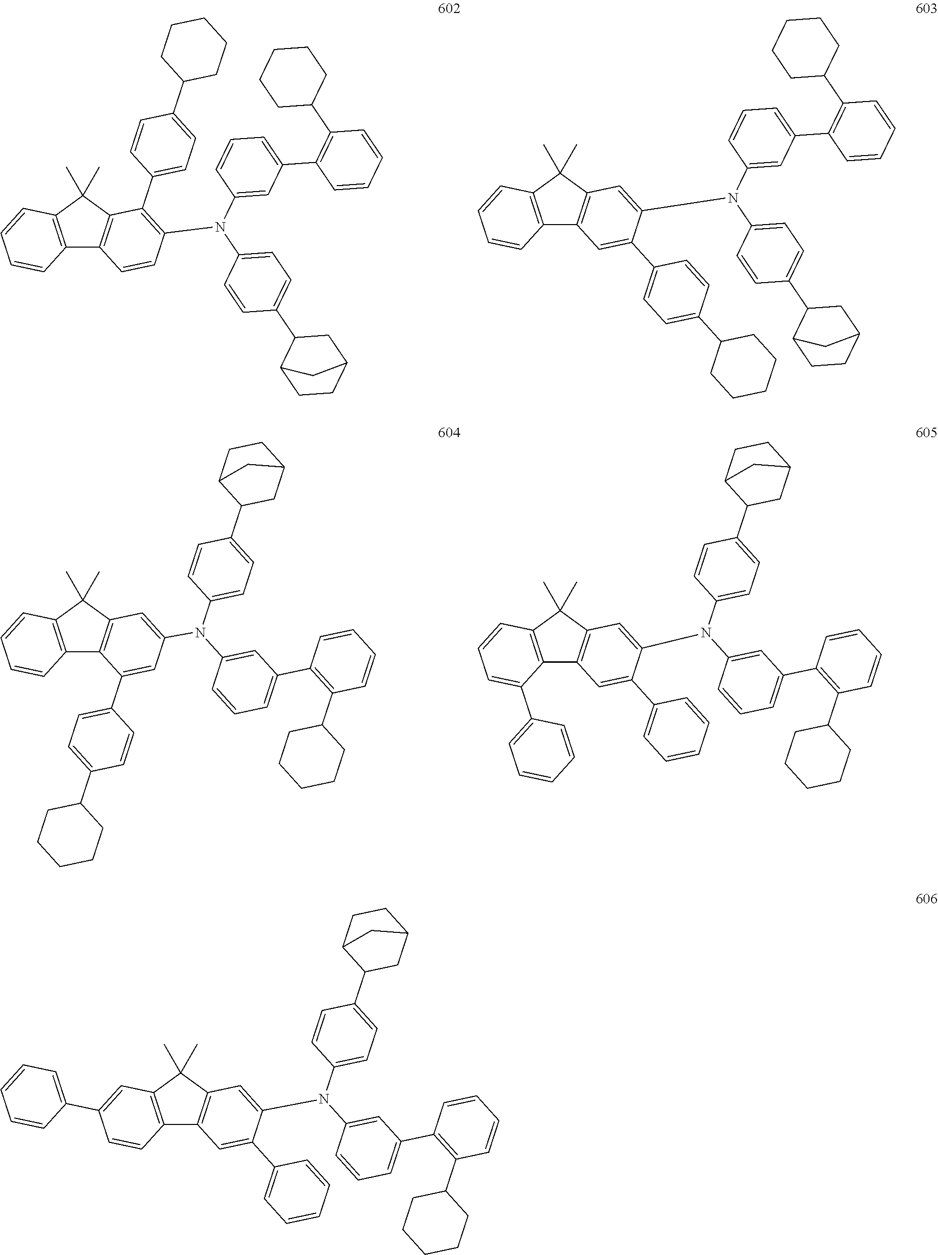

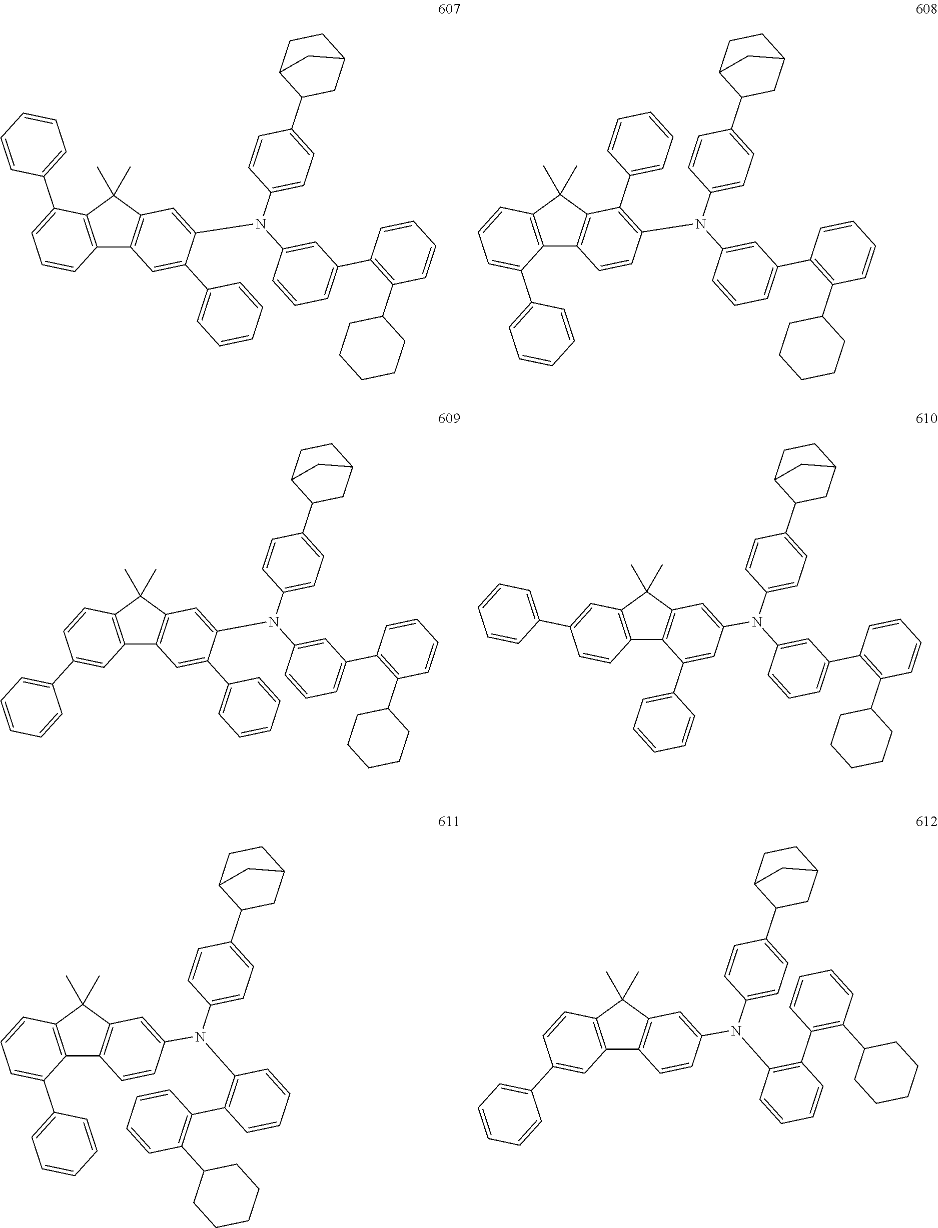

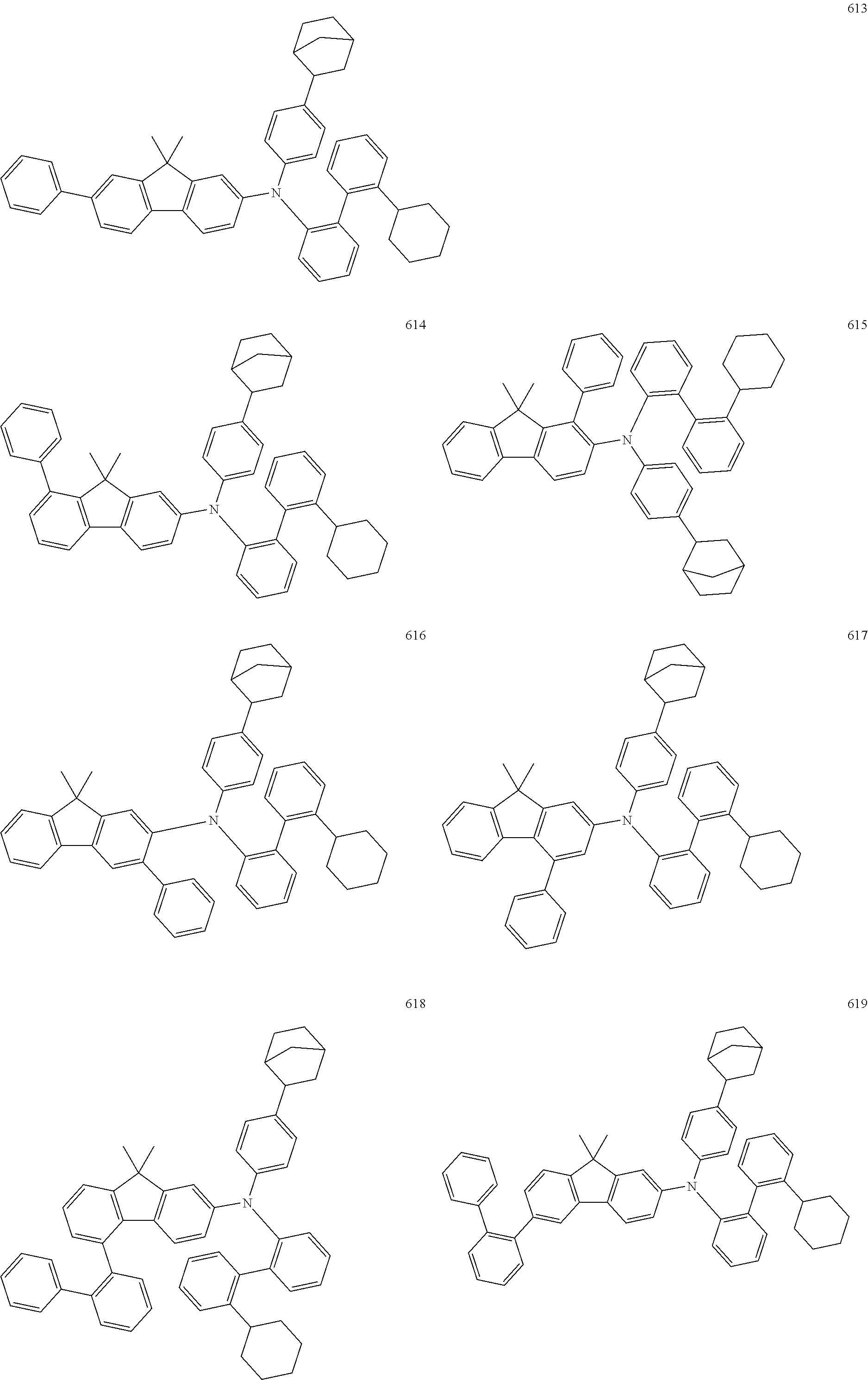

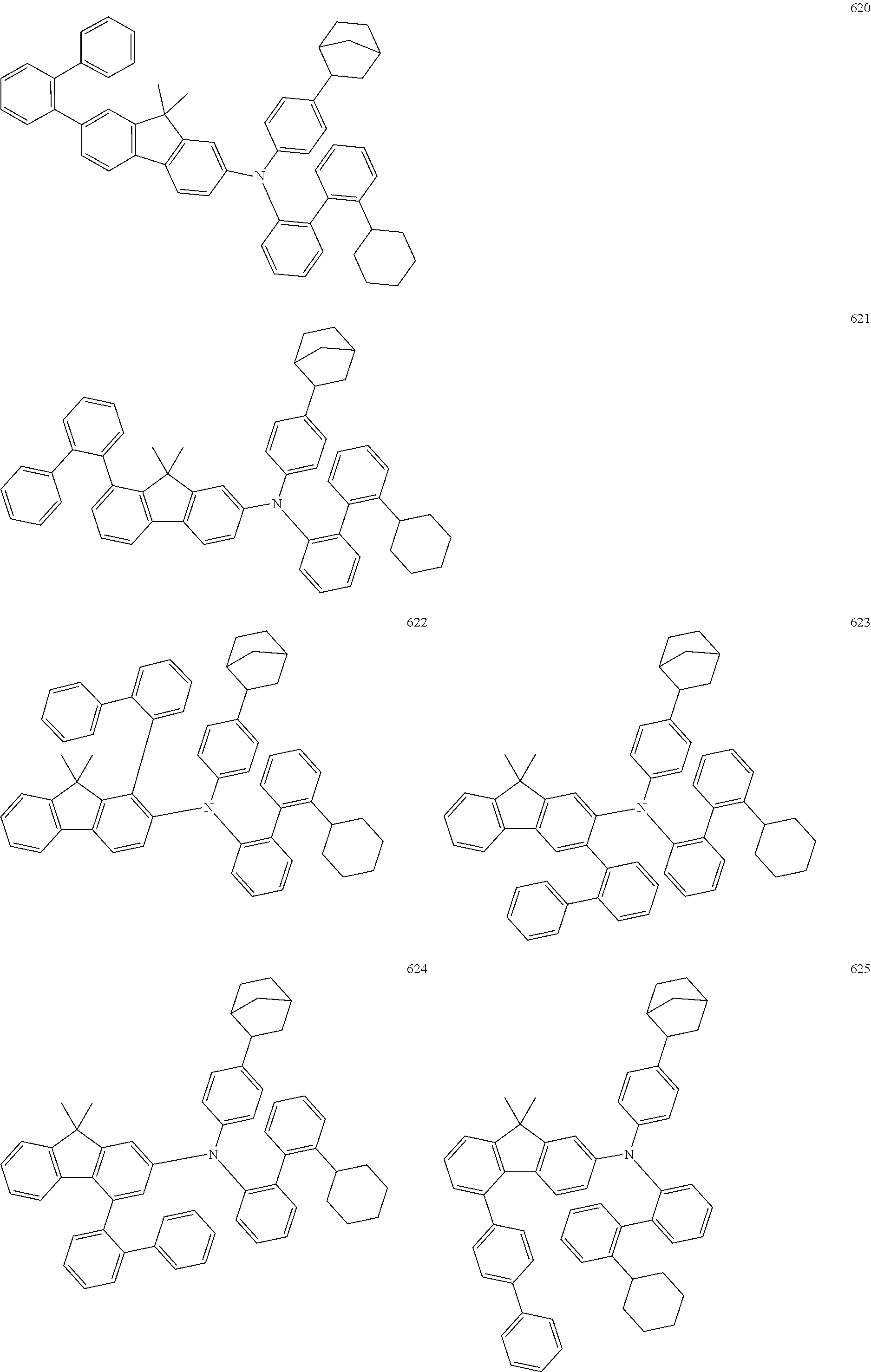

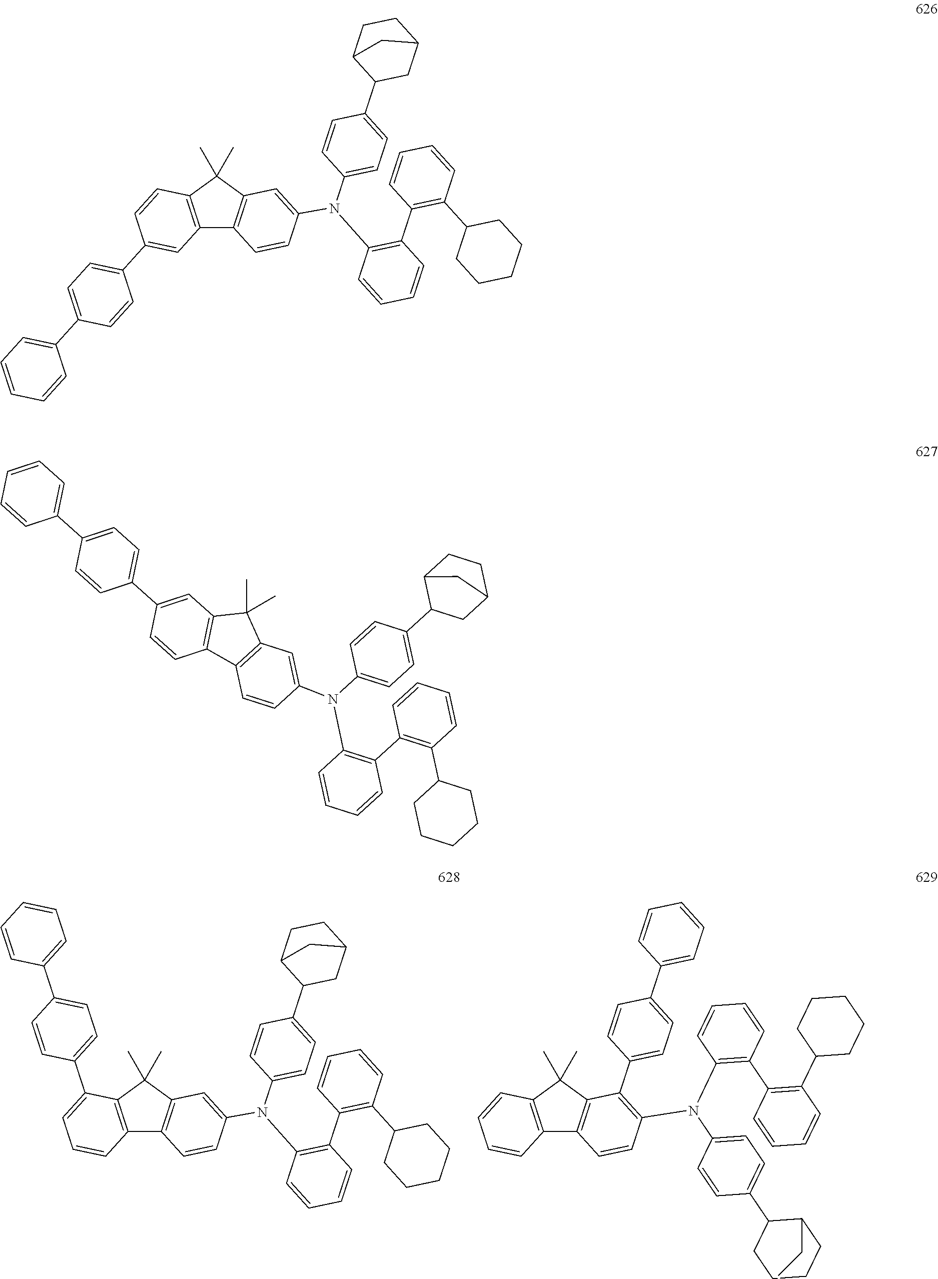

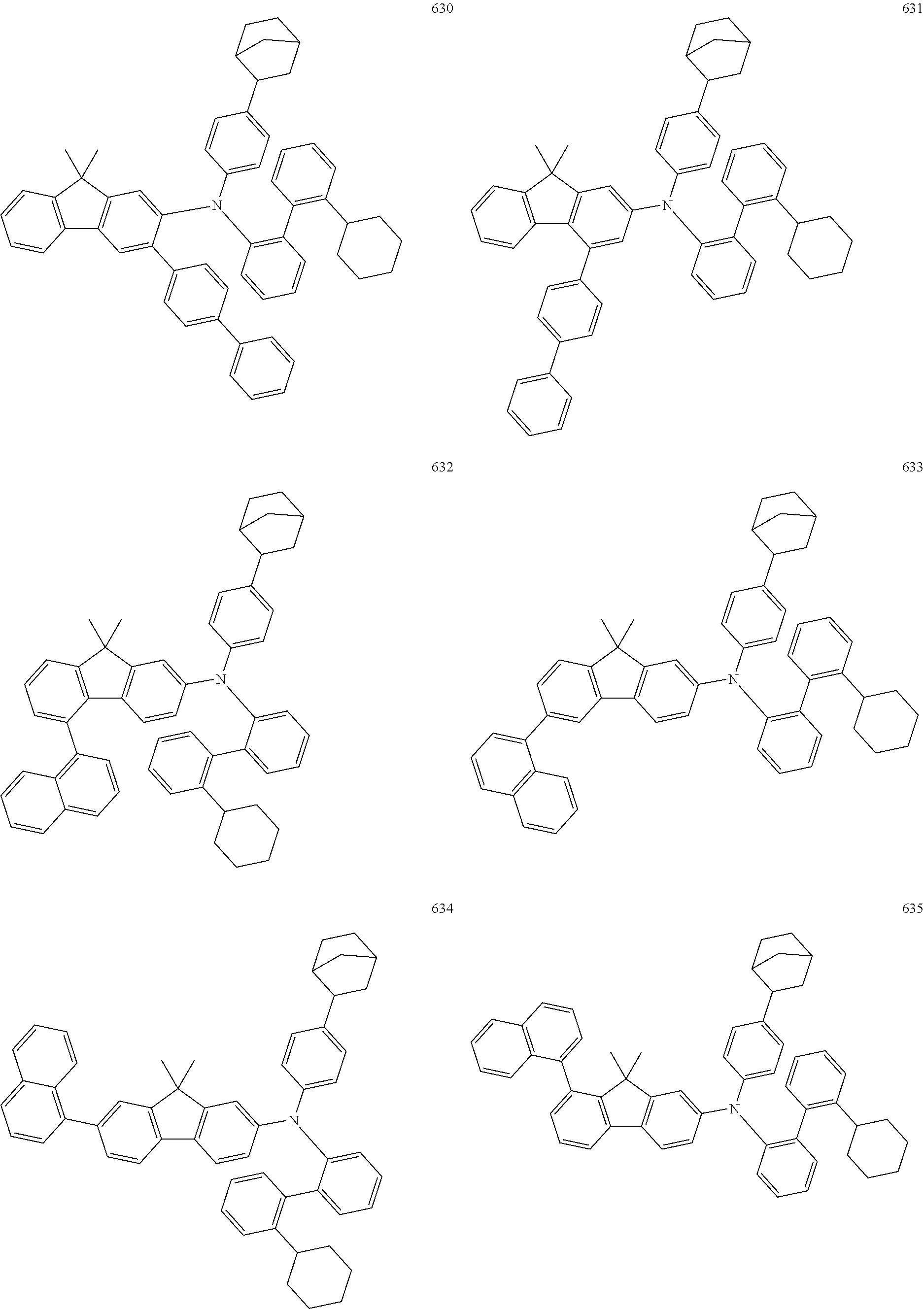

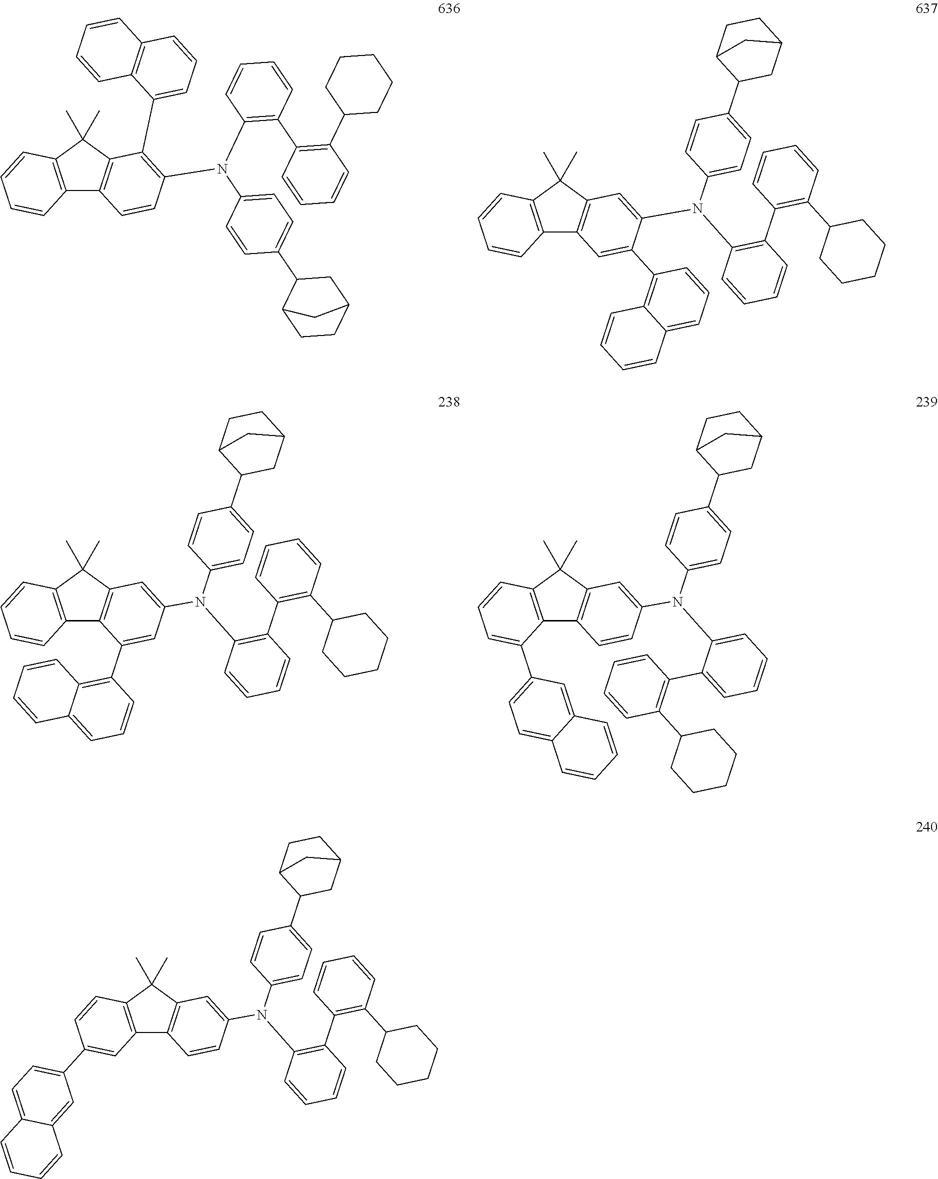 [SMILES: CC1(C)c2ccccc2-c2ccc(N(c3ccc(C4CC5CCC4C5)cc3)c3ccccc3-c3ccccc3C3CCCCC3)c(-c3cccc4ccccc34)c21 ; CC1(C)c2ccccc2-c2cc(-c3cccc4ccccc34)c(N(c3ccc(C4CC5CCC4C5)cc3)c3ccccc3-c3ccccc3C3CCCCC3)cc21 ; CC1(C)c2ccccc2-c2c(-c3cccc4ccccc34)cc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccccc3-c3ccccc3C3CCCCC3)cc21]

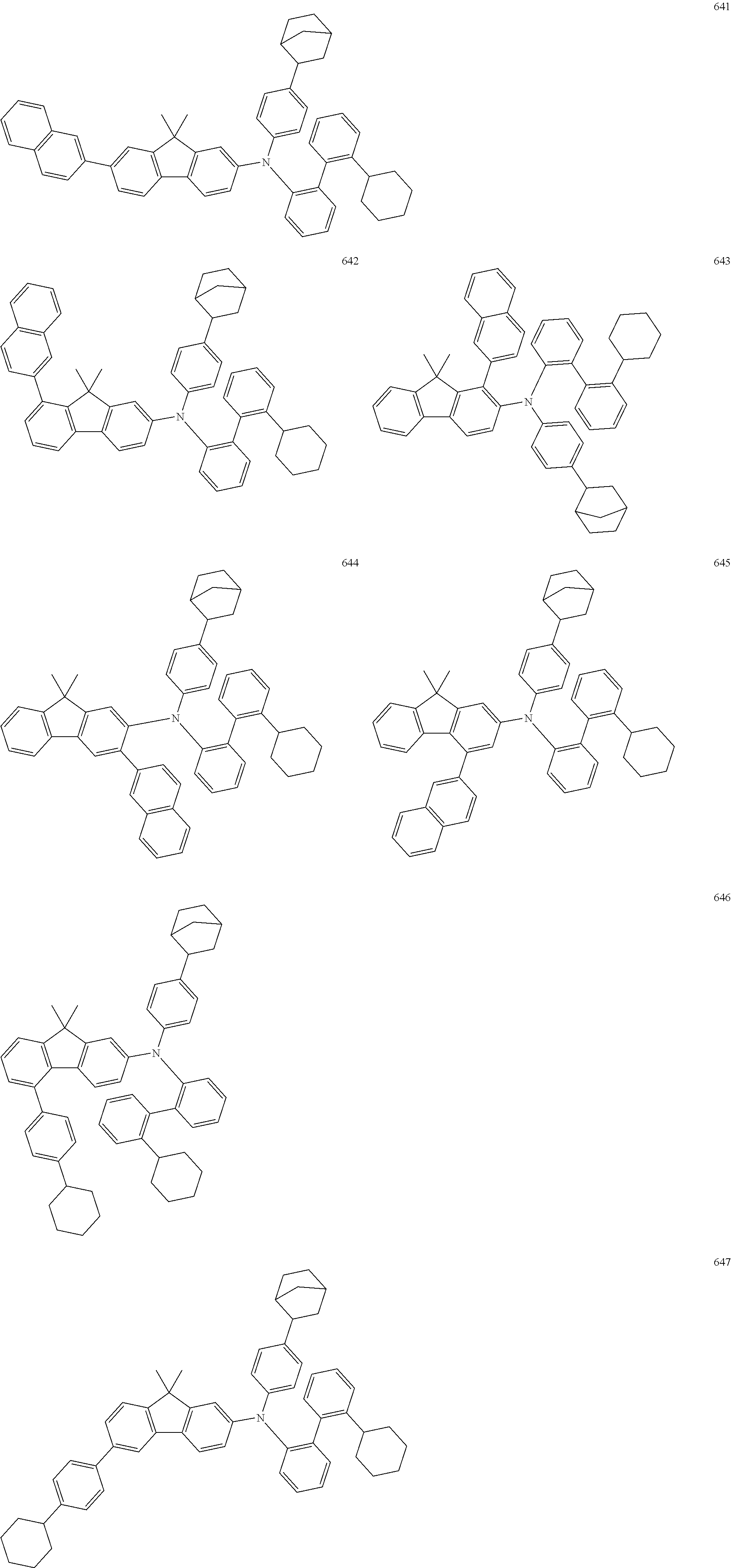

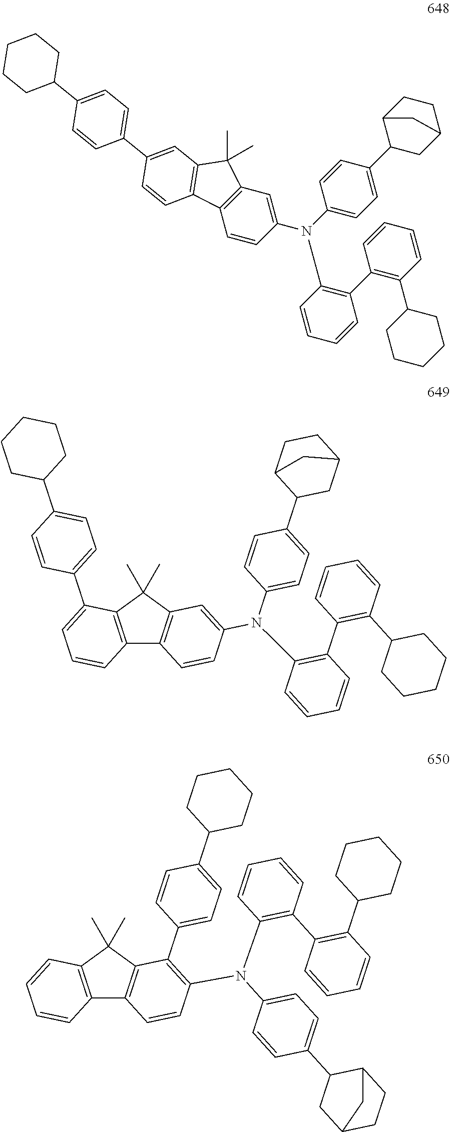 [SMILES: CC1(C)c2cc(-c3ccc(C4CCCCC4)cc3)ccc2-c2ccc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2cccc(-c3ccc(C4CCCCC4)cc3)c21 ; CC1(C)c2ccccc2-c2ccc(N(c3ccc(C4CC5CCC4C5)cc3)c3ccccc3-c3ccccc3C3CCCCC3)c(-c3ccc(C4CCCCC4)cc3)c21]

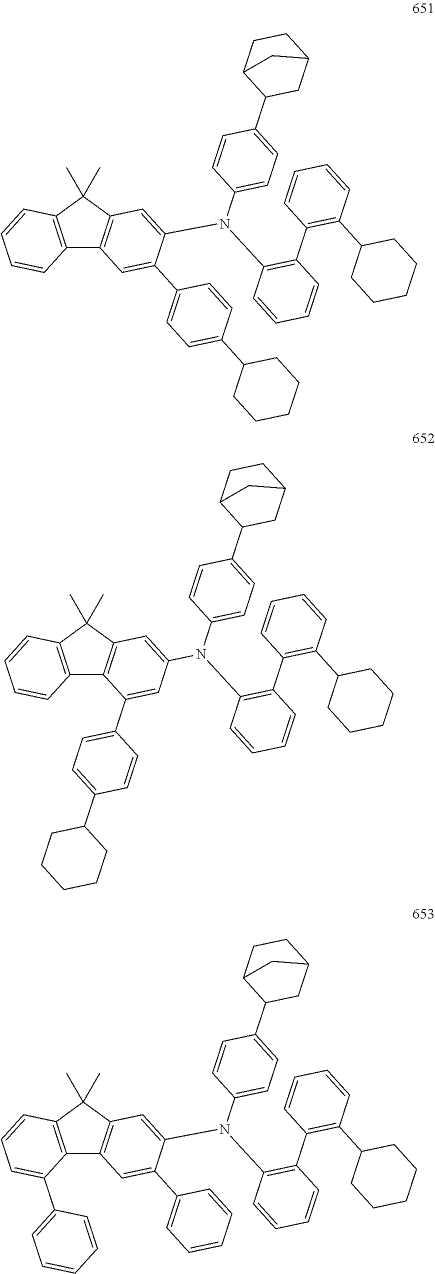

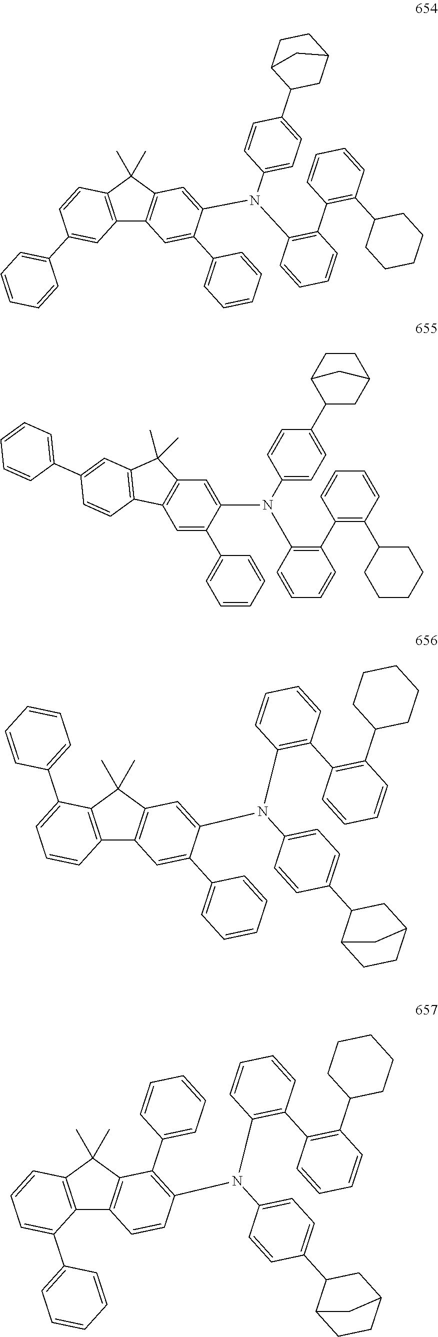 [SMILES: CC1(C)c2ccc(-c3ccccc3)cc2-c2cc(-c3ccccc3)c(N(c3ccc(C4CC5CCC4C5)cc3)c3ccccc3-c3ccccc3C3CCCCC3)cc21 ; CC1(C)c2cc(-c3ccccc3)ccc2-c2cc(-c3ccccc3)c(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3ccccc3-c3ccccc3C3CCCCC3)c(-c3ccccc3)c21]

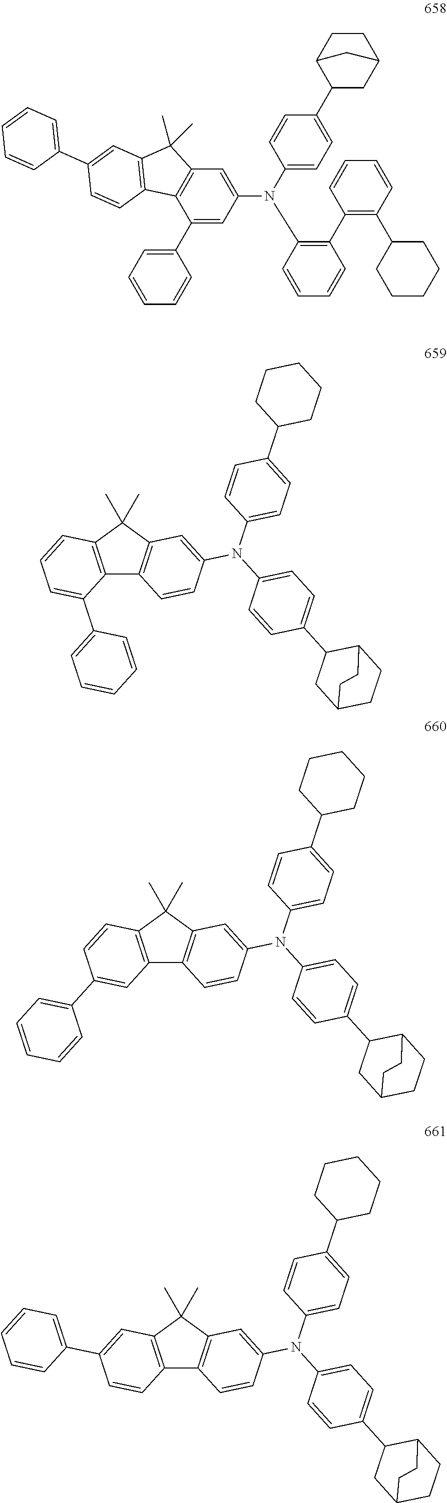

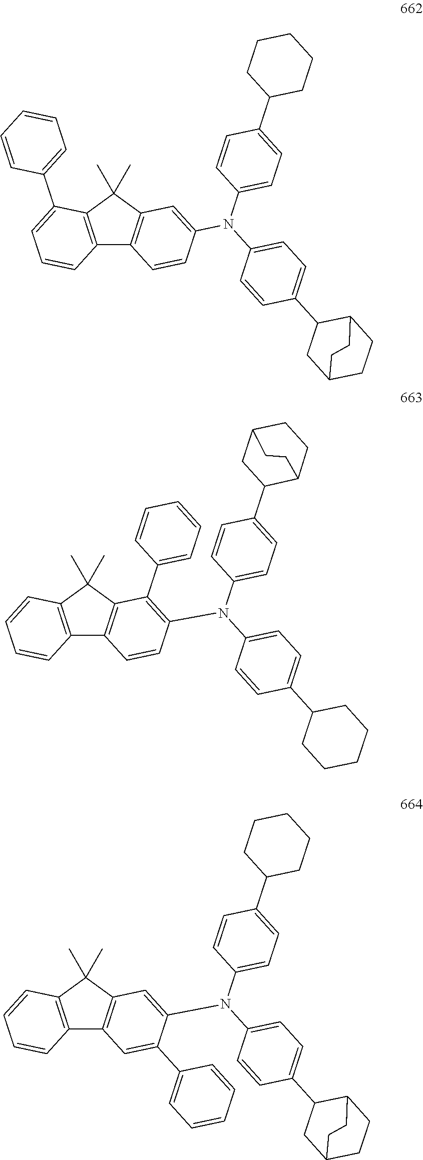 [SMILES: CC1(C)c2cc(N(c3ccc(C4CCCCC4)cc3)c3ccc(C4CC5CCC4CC5)cc3)ccc2-c2cccc(-c3ccccc3)c21 ; CC1(C)c2ccccc2-c2ccc(N(c3ccc(C4CCCCC4)cc3)c3ccc(C4CC5CCC4CC5)cc3)c(-c3ccccc3)c21 ; CC1(C)c2ccccc2-c2cc(-c3ccccc3)c(N(c3ccc(C4CCCCC4)cc3)c3ccc(C4CC5CCC4CC5)cc3)cc21]

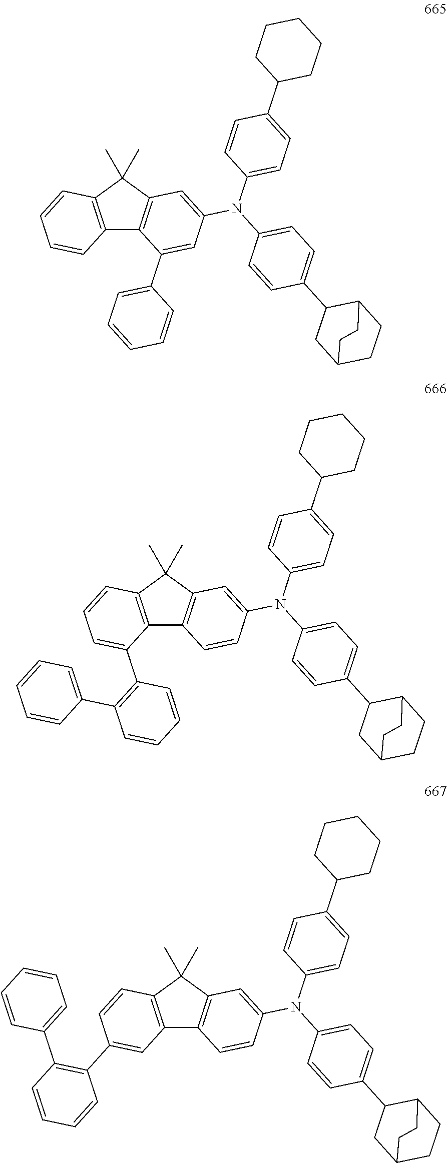 [SMILES: CC1(C)c2ccccc2-c2c(-c3ccccc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4CC5CCC4CC5)cc3)cc21]

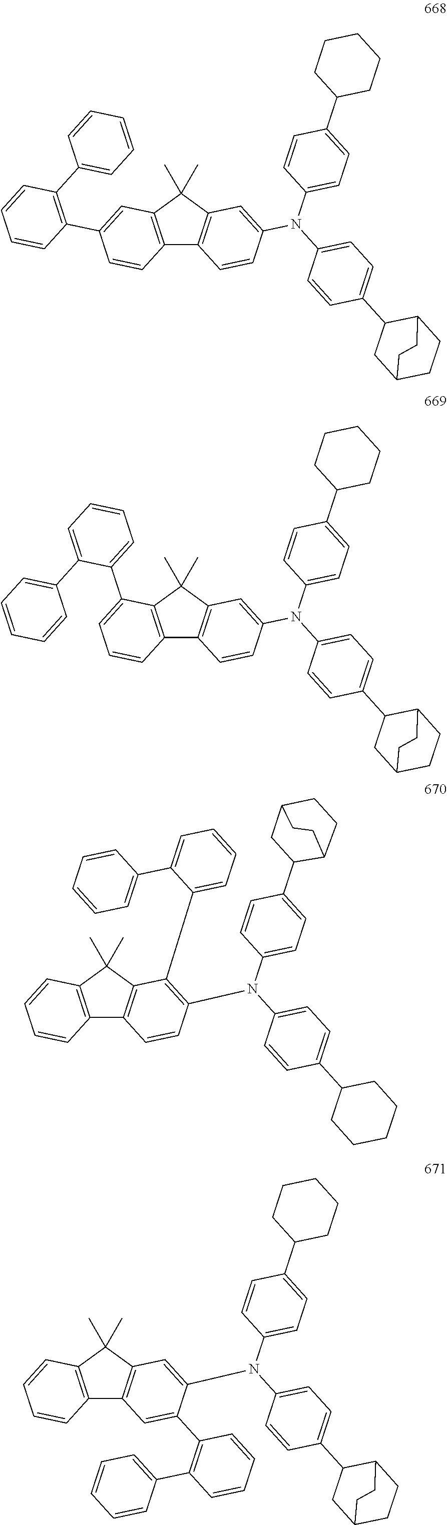 [SMILES: CC1(C)c2cc(-c3ccccc3-c3ccccc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4CC5)cc3)c(-c3ccccc3-c3ccccc3)c21 ; CC1(C)c2ccccc2-c2cc(-c3ccccc3-c3ccccc3)c(N(c3ccc(C4CCCCC4)cc3)c3ccc(C4CC5CCC4CC5)cc3)cc21]

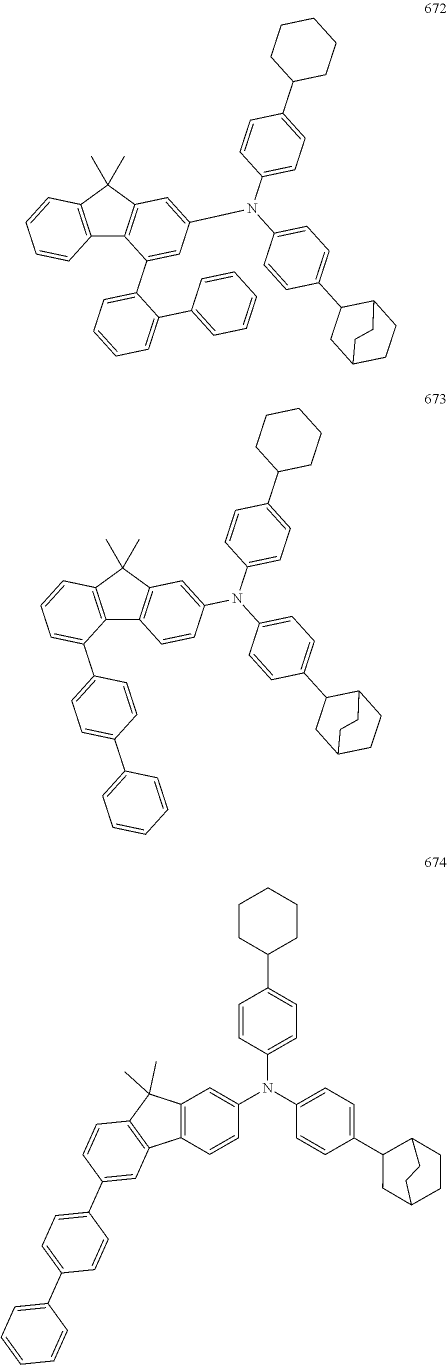 [SMILES: CC1(C)c2ccccc2-c2c(-c3ccccc3-c3ccccc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4CC5CCC4CC5)cc3)cc21]

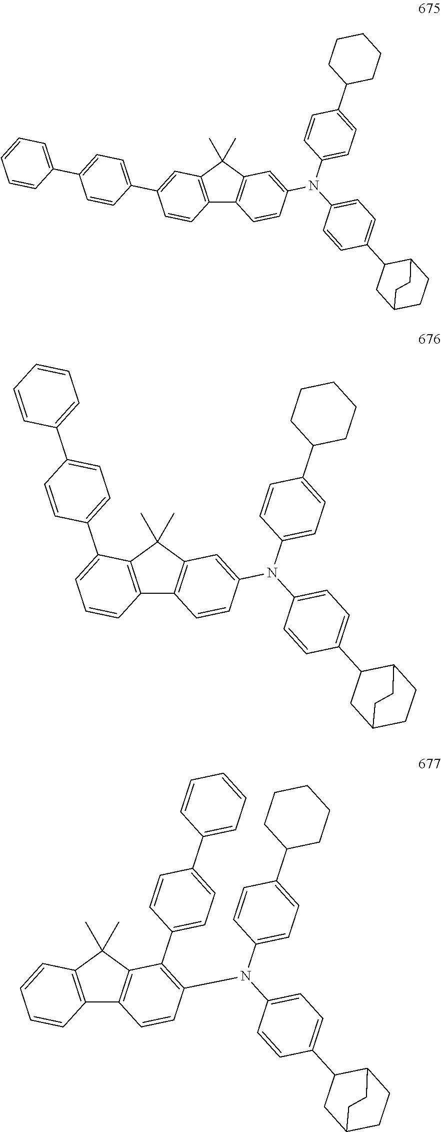 [SMILES: CC1(C)c2cc(-c3ccc(-c4ccccc4)cc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc(-c4ccccc4)cc3)c21 ; CC1(C)c2ccccc2-c2ccc(N(c3ccc(C4CCCCC4)cc3)c3ccc(C4CC5CCC4CC5)cc3)c(-c3ccc(-c4ccccc4)cc3)c21]

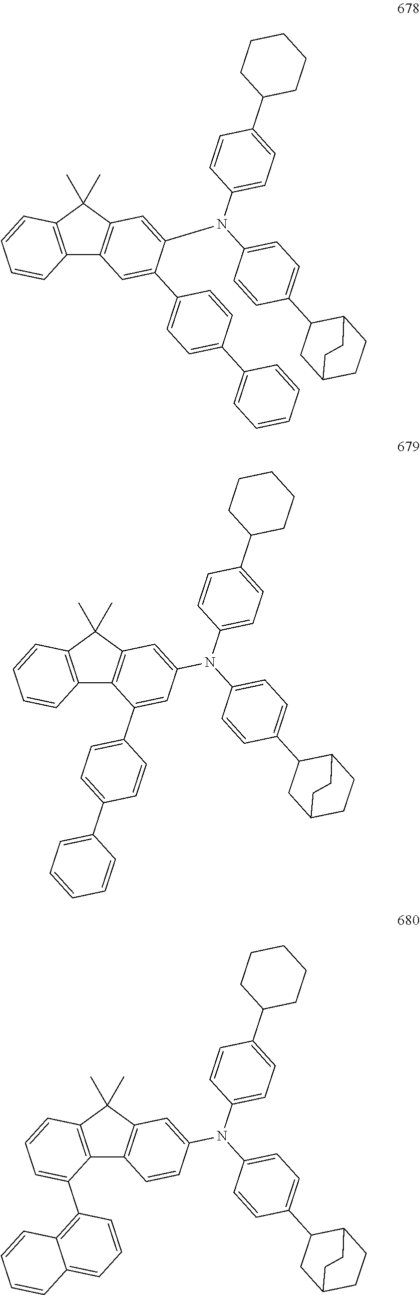

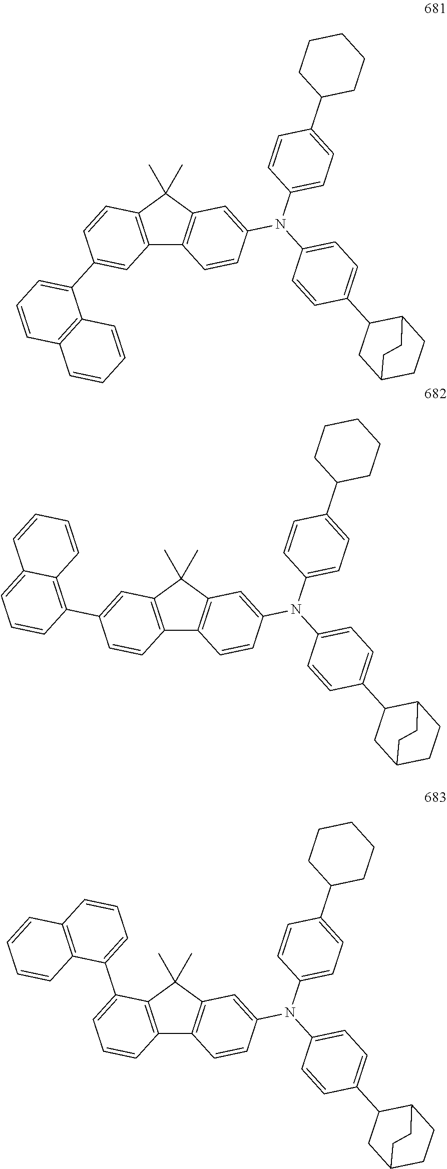

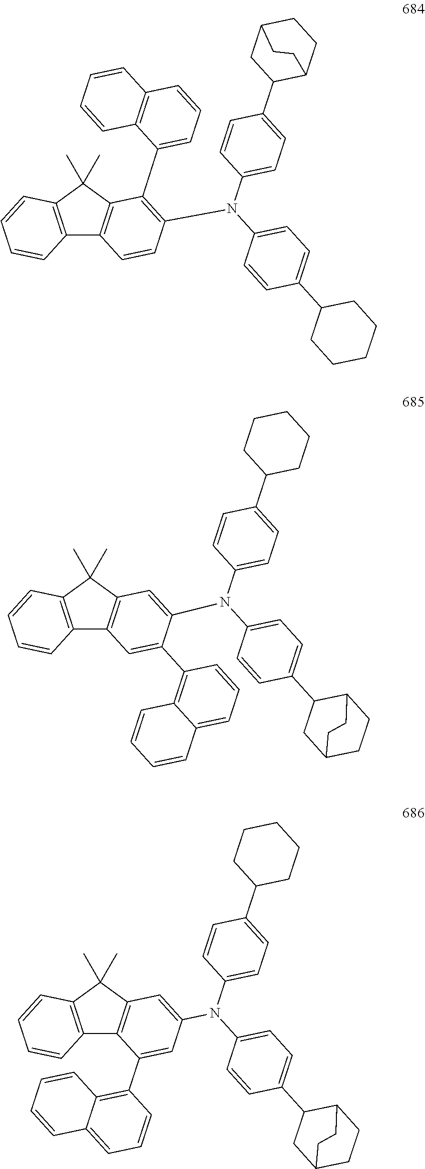 [SMILES: CC1(C)c2ccccc2-c2ccc(N(c3ccc(C4CCCCC4)cc3)c3ccc(C4CC5CCC4CC5)cc3)c(-c3cccc4ccccc34)c21 ; CC1(C)c2ccccc2-c2cc(-c3cccc4ccccc34)c(N(c3ccc(C4CCCCC4)cc3)c3ccc(C4CC5CCC4CC5)cc3)cc21 ; CC1(C)c2ccccc2-c2c(-c3cccc4ccccc34)cc(N(c3ccc(C4CCCCC4)cc3)c3ccc(C4CC5CCC4CC5)cc3)cc21]

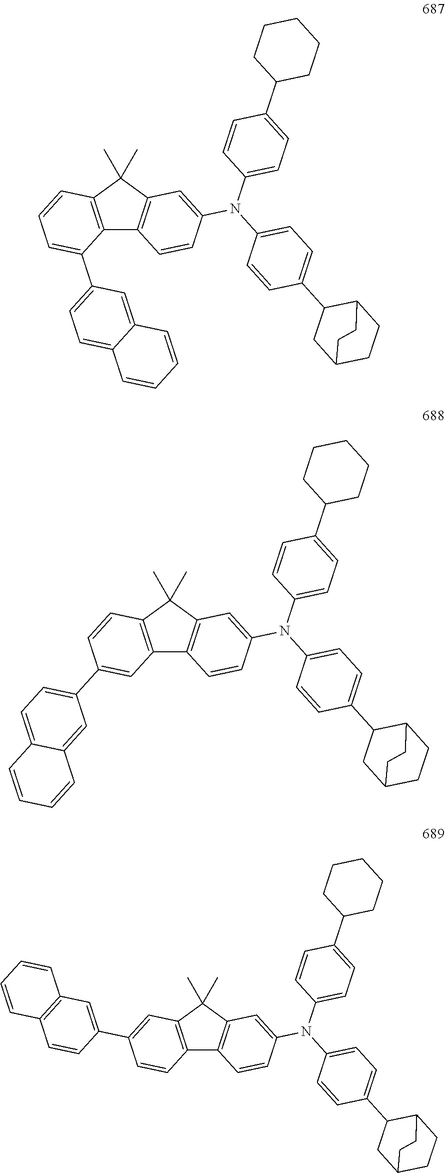 [SMILES: CC1(C)c2cc(N(c3ccc(C4CCCCC4)cc3)c3ccc(C4CC5CCC4CC5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CC5CCC4CC5)cc3)cc21 ; CC1(C)c2cc(-c3ccc4ccccc4c3)ccc2-c2ccc(N(c3ccc(C4CCCCC4)cc3)c3ccc(C4CC5CCC4CC5)cc3)cc21]

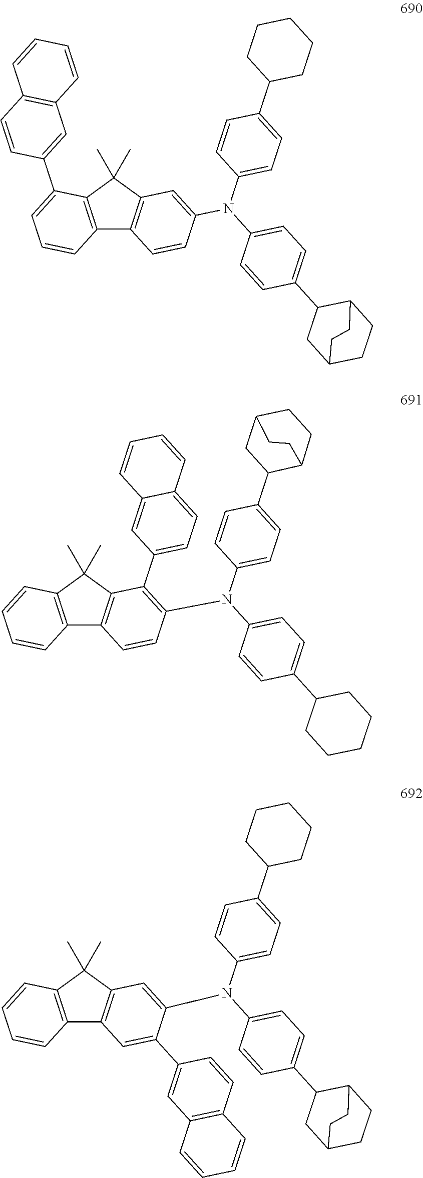 [SMILES: CC1(C)c2cc(N(c3ccc(C4CCCCC4)cc3)c3ccc(C4CC5CCC4CC5)cc3)ccc2-c2cccc(-c3ccc4ccccc4c3)c21 ; CC1(C)c2ccccc2-c2ccc(N(c3ccc(C4CCCCC4)cc3)c3ccc(C4CC5CCC4CC5)cc3)c(-c3ccc4ccccc4c3)c21 ; CC1(C)c2ccccc2-c2cc(-c3ccc4ccccc4c3)c(N(c3ccc(C4CCCCC4)cc3)c3ccc(C4CC5CCC4CC5)cc3)cc21]

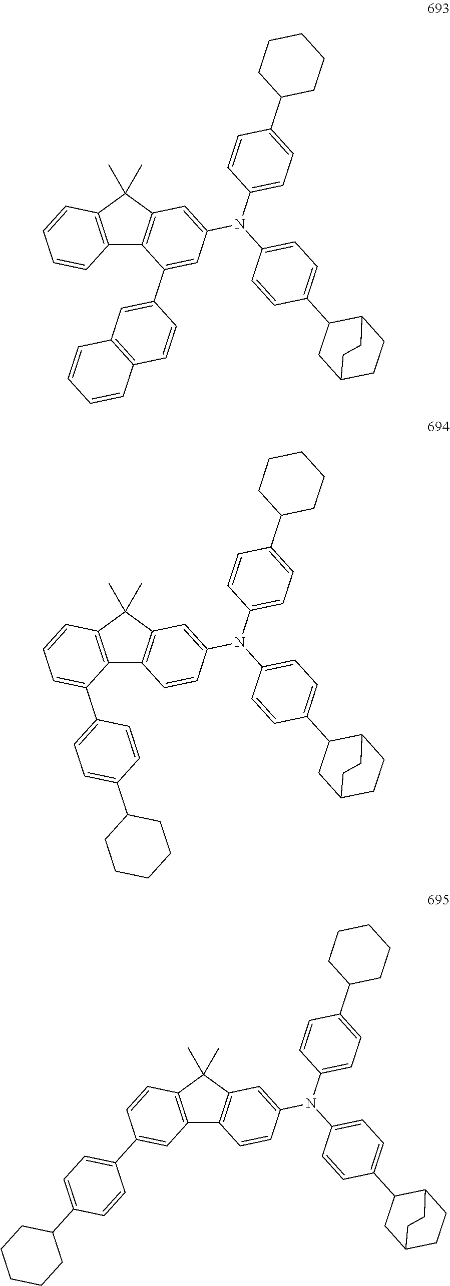 [SMILES: CC1(C)c2ccccc2-c2c(-c3ccc4ccccc4c3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CCCCC4)cc3)c3ccc(C4CC5CCC4CC5)cc3)cc21]

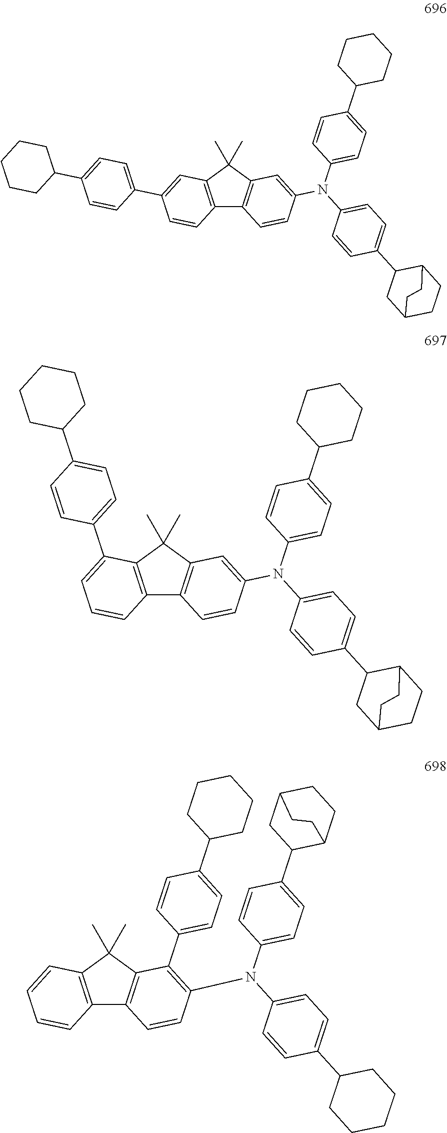 [SMILES: CC1(C)c2cc(-c3ccc(C4CCCCC4)cc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc(C4CCCCC4)cc3)c21 ; CC1(C)c2ccccc2-c2ccc(N(c3ccc(C4CCCCC4)cc3)c3ccc(C4CC5CCC4CC5)cc3)c(-c3ccc(C4CCCCC4)cc3)c21]

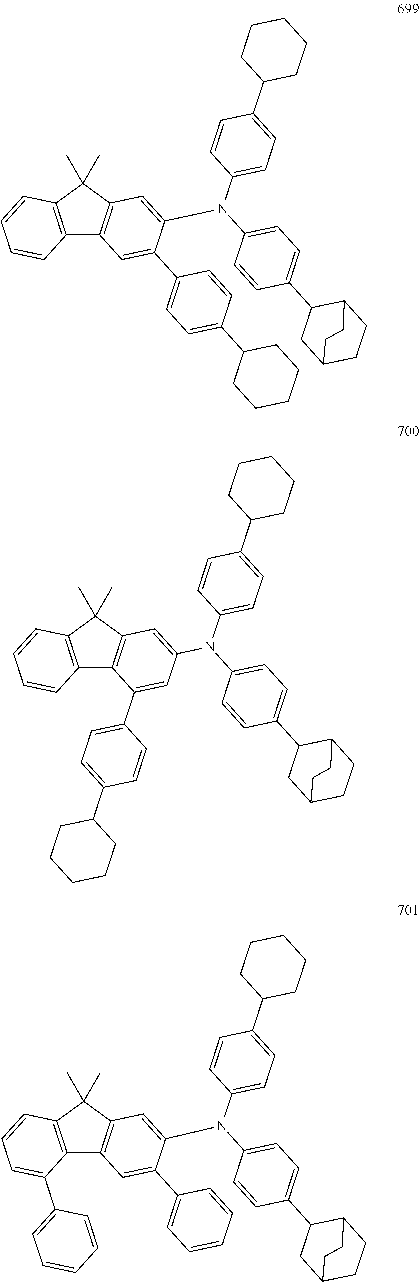 [SMILES: CC1(C)c2ccccc2-c2cc(-c3ccc(C4CCCCC4)cc3)c(N(c3ccc(C4CCCCC4)cc3)c3ccc(C4CC5CCC4CC5)cc3)cc21 ; CC1(C)c2ccccc2-c2c(-c3ccc(C4CCCCC4)cc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)c(-c3ccccc3)cc2-c2c(-c3ccccc3)cccc21]

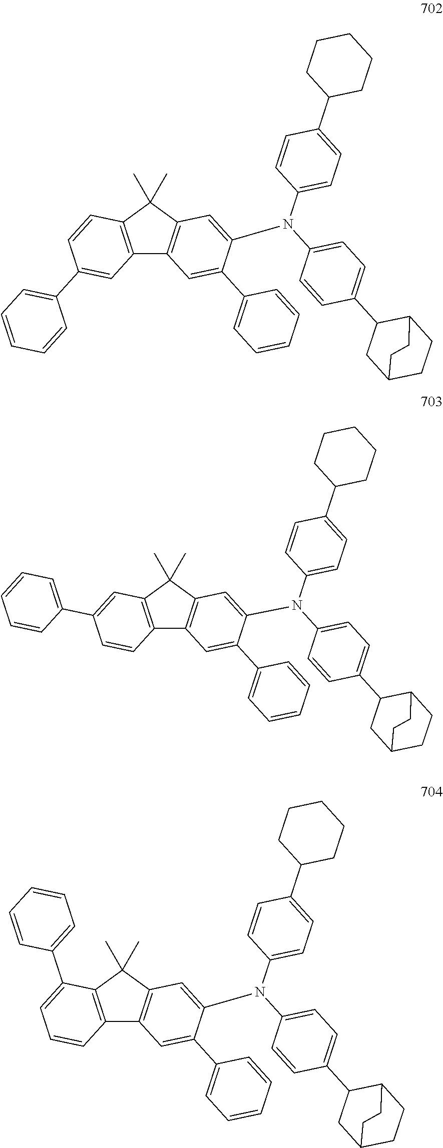 [SMILES: CC1(C)c2ccc(-c3ccccc3)cc2-c2cc(-c3ccccc3)c(N(c3ccc(C4CCCCC4)cc3)c3ccc(C4CC5CCC4CC5)cc3)cc21 ; CC1(C)c2cc(-c3ccccc3)ccc2-c2cc(-c3ccccc3)c(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)c(-c3ccccc3)cc2-c2cccc(-c3ccccc3)c21]

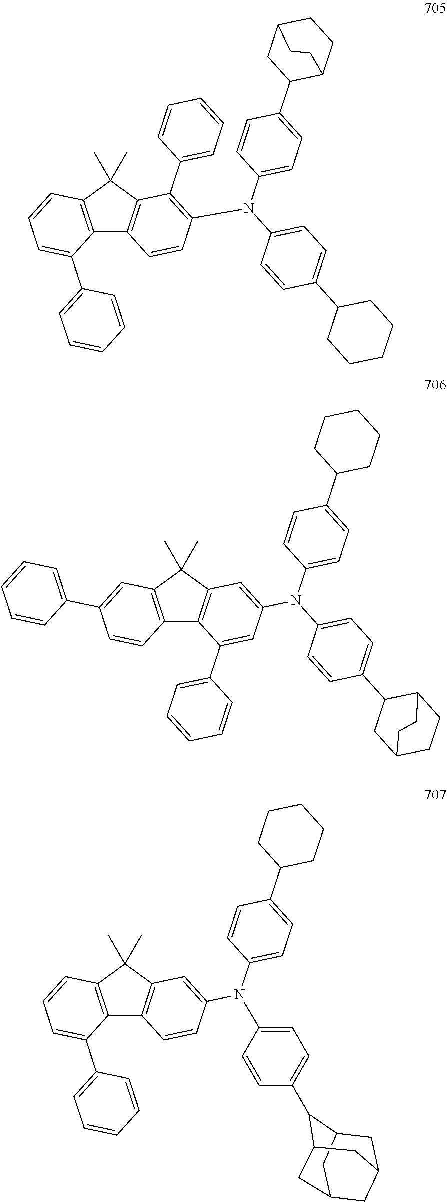 [SMILES: CC1(C)c2cccc(-c3ccccc3)c2-c2ccc(N(c3ccc(C4CCCCC4)cc3)c3ccc(C4CC5CCC4CC5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4C5CC6CC(C5)CC4C6)cc3)ccc2-c2c(-c3ccccc3)cccc21]

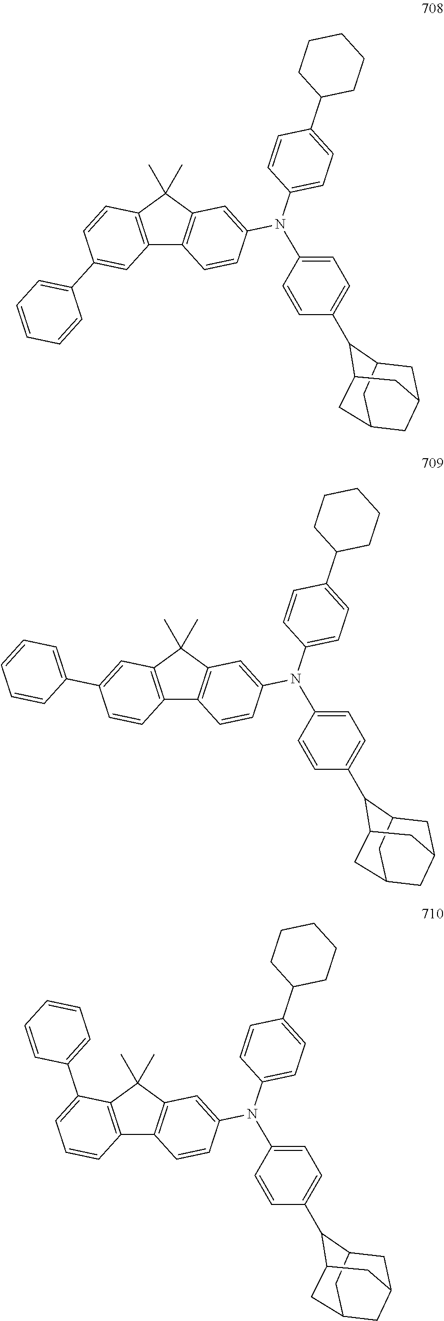 [SMILES: CC1(C)c2ccc(-c3ccccc3)cc2-c2ccc(N(c3ccc(C4CCCCC4)cc3)c3ccc(C4C5CC6CC(C5)CC4C6)cc3)cc21 ; CC1(C)c2cc(-c3ccccc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccccc3)c21]

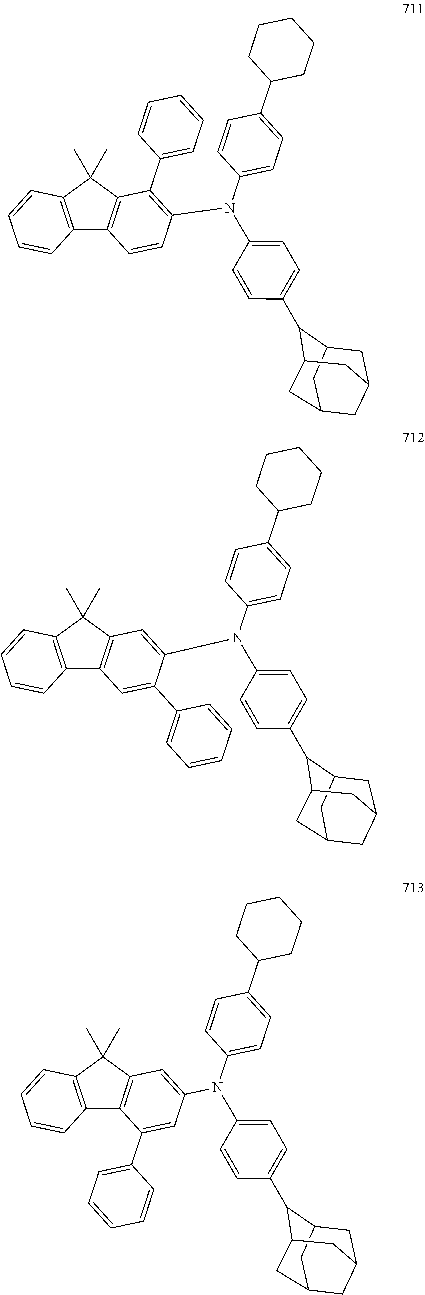

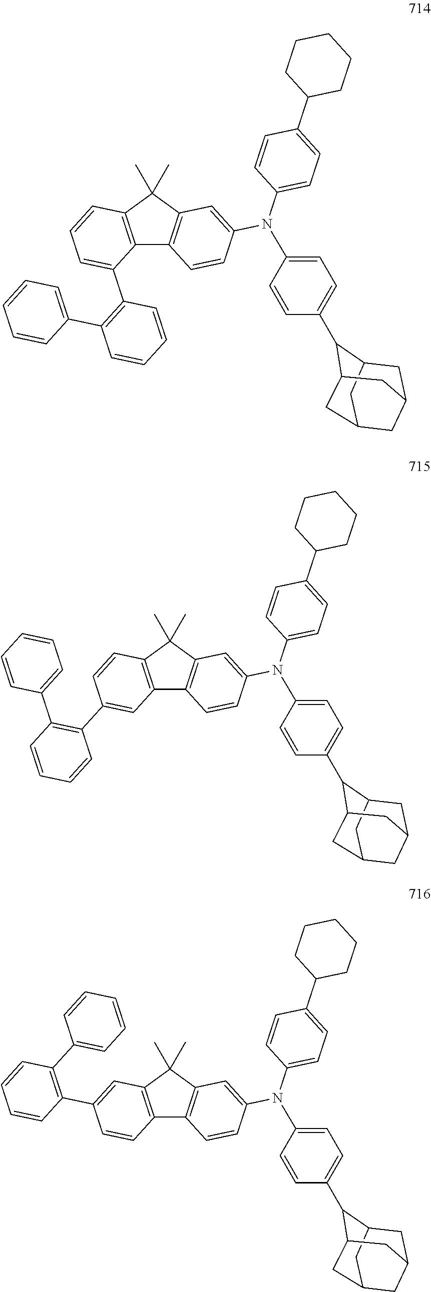 [SMILES: CC1(C)c2cc(N(c3ccc(C4CCCCC4)cc3)c3ccc(C4C5CC6CC(C5)CC4C6)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4C5CC6CC(C5)CC4C6)cc3)cc21 ; CC1(C)c2cc(-c3ccccc3-c3ccccc3)ccc2-c2ccc(N(c3ccc(C4CCCCC4)cc3)c3ccc(C4C5CC6CC(C5)CC4C6)cc3)cc21]

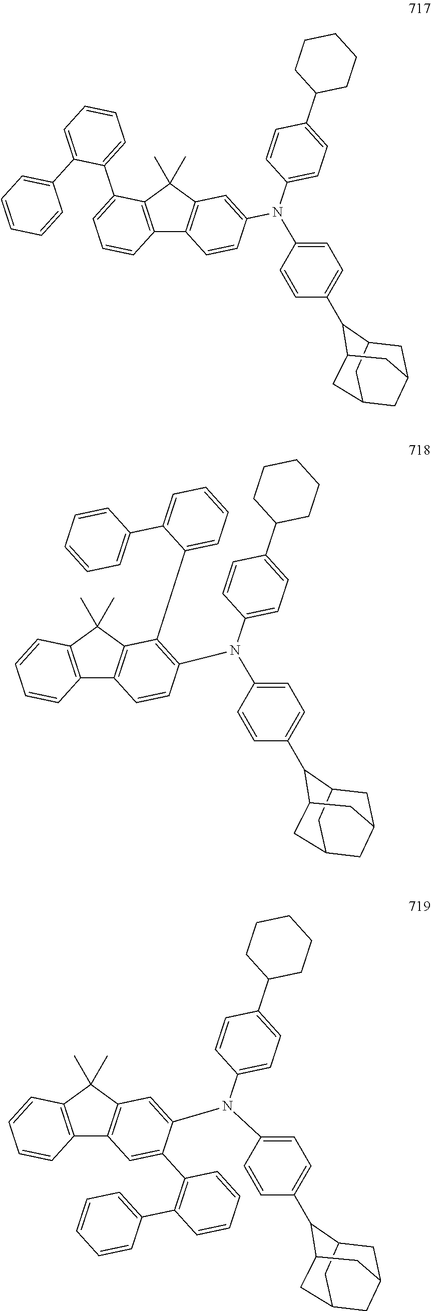 [SMILES: CC1(C)c2cc(N(c3ccc(C4CCCCC4)cc3)c3ccc(C4C5CC6CC(C5)CC4C6)cc3)ccc2-c2cccc(-c3ccccc3-c3ccccc3)c21 ; CC1(C)c2ccccc2-c2ccc(N(c3ccc(C4CCCCC4)cc3)c3ccc(C4C5CC6CC(C5)CC4C6)cc3)c(-c3ccccc3-c3ccccc3)c21 ; CC1(C)c2ccccc2-c2cc(-c3ccccc3-c3ccccc3)c(N(c3ccc(C4CCCCC4)cc3)c3ccc(C4C5CC6CC(C5)CC4C6)cc3)cc21]

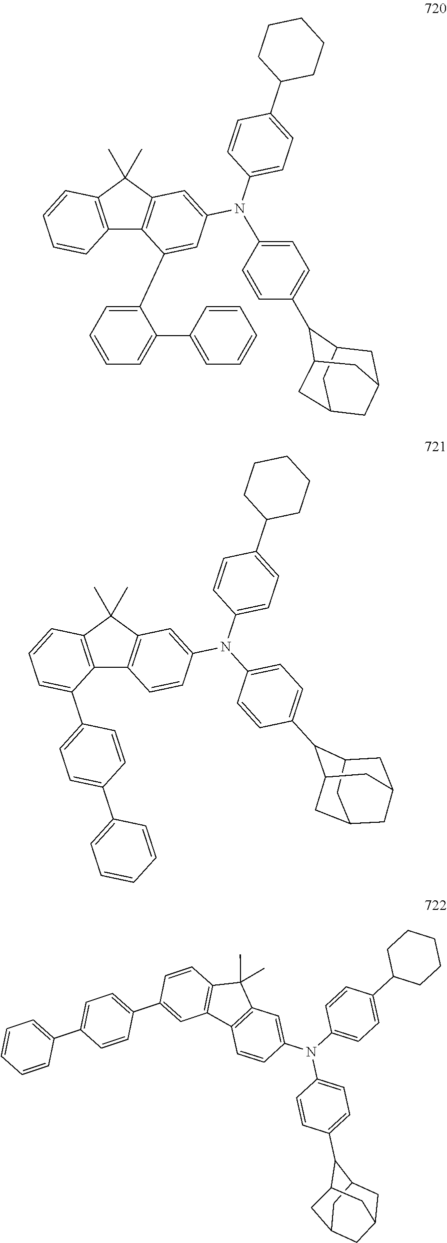 [SMILES: CC1(C)c2ccccc2-c2c(-c3ccccc3-c3ccccc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4C5CC6CC(C5)CC4C6)cc3)cc21]

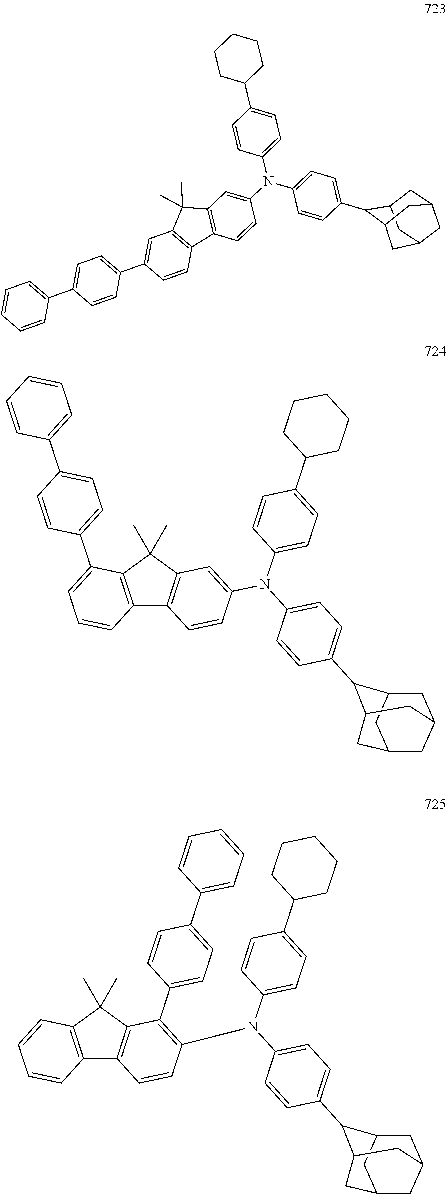

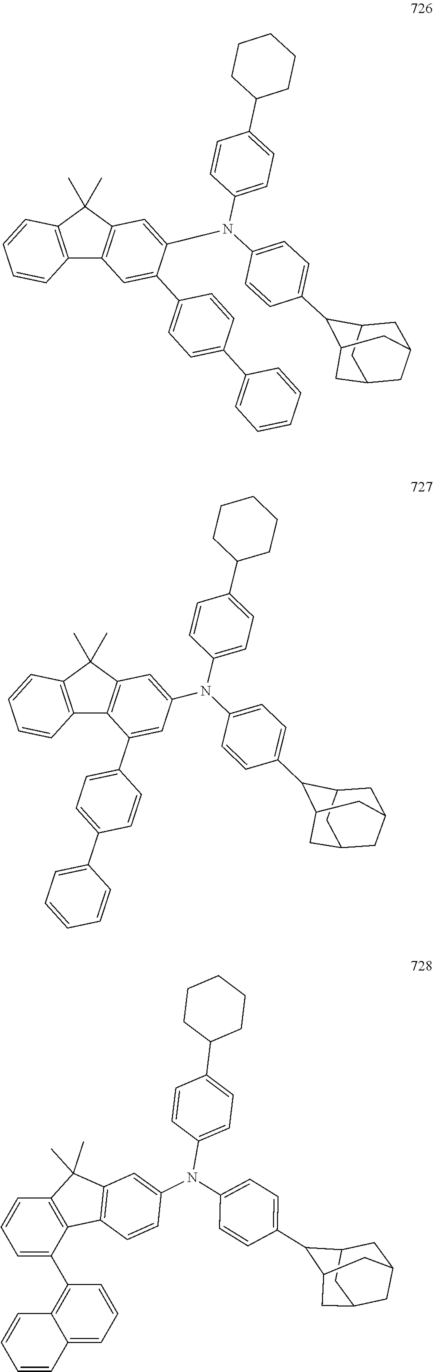 [SMILES: CC1(C)c2ccccc2-c2cc(-c3ccc(-c4ccccc4)cc3)c(N(c3ccc(C4CCCCC4)cc3)c3ccc(C4C5CC6CC(C5)CC4C6)cc3)cc21 ; CC1(C)c2ccccc2-c2c(-c3ccc(-c4ccccc4)cc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3cccc4ccccc34)cccc21]

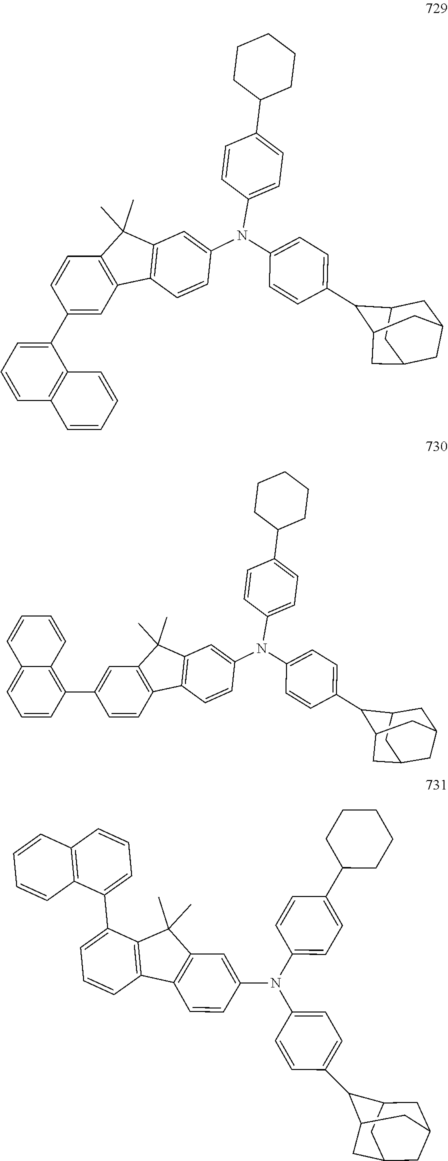 [SMILES: CC1(C)c2ccc(-c3cccc4ccccc34)cc2-c2ccc(N(c3ccc(C4CCCCC4)cc3)c3ccc(C4C5CC6CC(C5)CC4C6)cc3)cc21 ; CC1(C)c2cc(-c3cccc4ccccc34)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3cccc4ccccc34)c21]

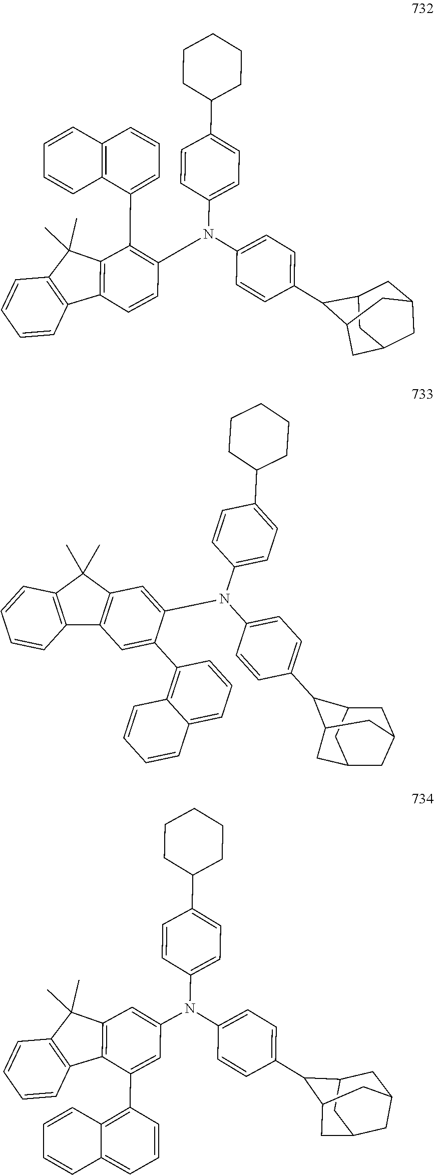 [SMILES: CC1(C)c2ccccc2-c2ccc(N(c3ccc(C4CCCCC4)cc3)c3ccc(C4C5CC6CC(C5)CC4C6)cc3)c(-c3cccc4ccccc34)c21 ; CC1(C)c2ccccc2-c2cc(-c3cccc4ccccc34)c(N(c3ccc(C4CCCCC4)cc3)c3ccc(C4C5CC6CC(C5)CC4C6)cc3)cc21 ; CC1(C)c2ccccc2-c2c(-c3cccc4ccccc34)cc(N(c3ccc(C4CCCCC4)cc3)c3ccc(C4C5CC6CC(C5)CC4C6)cc3)cc21]

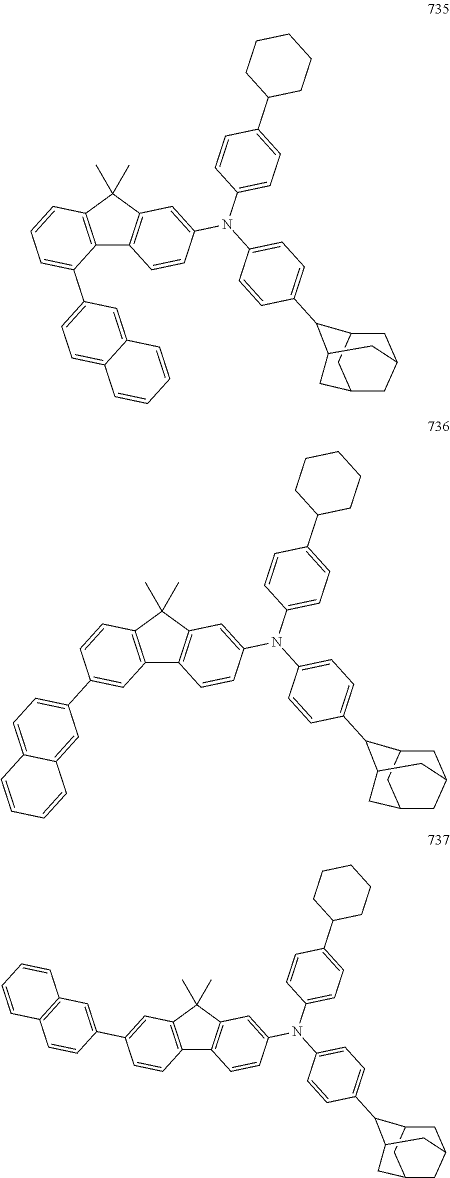 [SMILES: CC1(C)c2cc(N(c3ccc(C4CCCCC4)cc3)c3ccc(C4C5CC6CC(C5)CC4C6)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4C5CC6CC(C5)CC4C6)cc3)cc21 ; CC1(C)c2cc(-c3ccc4ccccc4c3)ccc2-c2ccc(N(c3ccc(C4CCCCC4)cc3)c3ccc(C4C5CC6CC(C5)CC4C6)cc3)cc21]

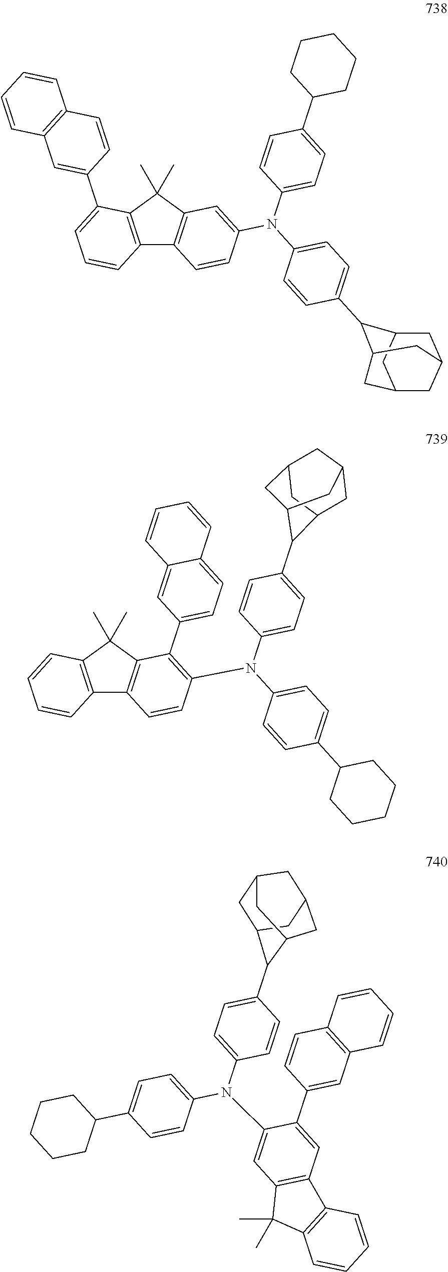 [SMILES: CC1(C)c2cc(N(c3ccc(C4CCCCC4)cc3)c3ccc(C4C5CC6CC(C5)CC4C6)cc3)ccc2-c2cccc(-c3ccc4ccccc4c3)c21 ; CC1(C)c2ccccc2-c2ccc(N(c3ccc(C4CCCCC4)cc3)c3ccc(C4C5CC6CC(C5)CC4C6)cc3)c(-c3ccc4ccccc4c3)c21 ; CC1(C)c2ccccc2-c2cc(-c3ccc4ccccc4c3)c(N(c3ccc(C4CCCCC4)cc3)c3ccc(C4C5CC6CC(C5)CC4C6)cc3)cc21]

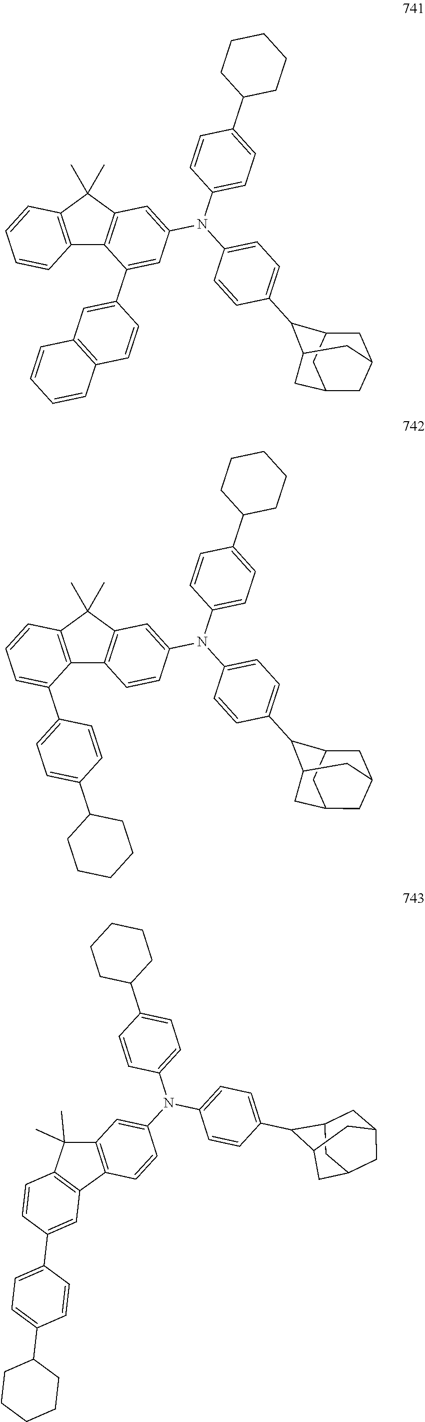

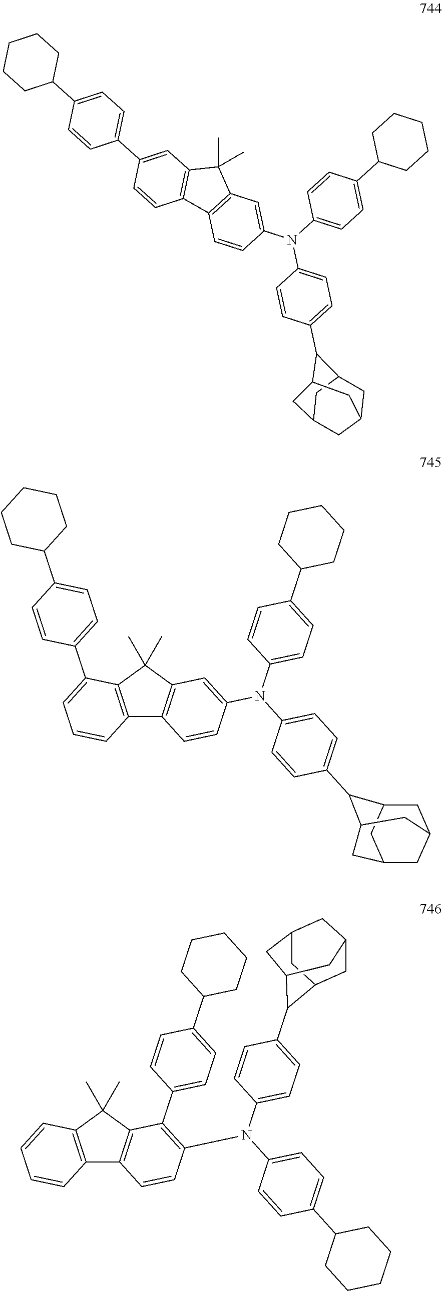 [SMILES: CC1(C)c2cc(-c3ccc(C4CCCCC4)cc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc(C4CCCCC4)cc3)c21 ; CC1(C)c2ccccc2-c2ccc(N(c3ccc(C4CCCCC4)cc3)c3ccc(C4C5CC6CC(C5)CC4C6)cc3)c(-c3ccc(C4CCCCC4)cc3)c21]

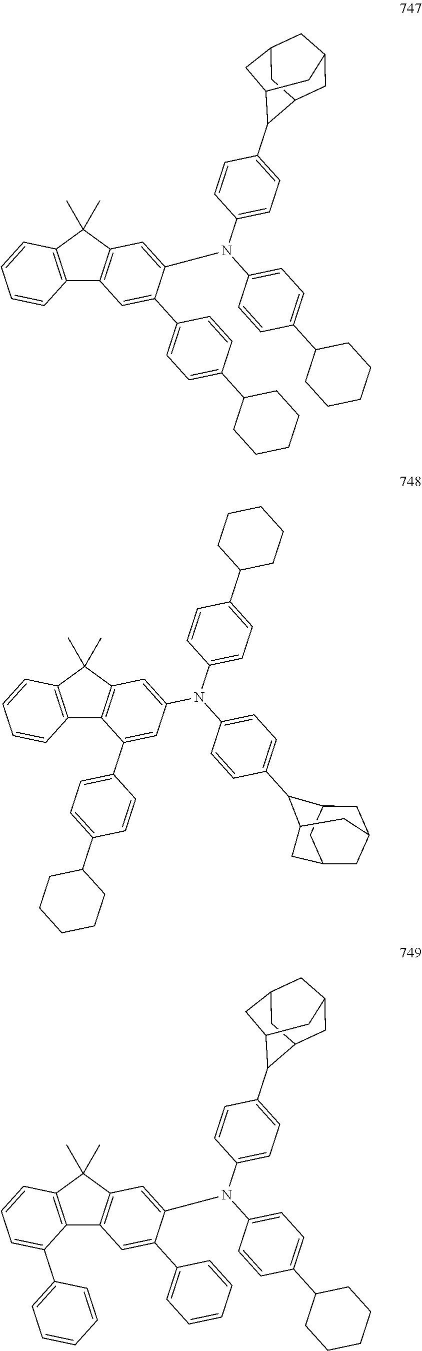 [SMILES: CC1(C)c2ccccc2-c2cc(-c3ccc(C4CCCCC4)cc3)c(N(c3ccc(C4CCCCC4)cc3)c3ccc(C4C5CC6CC(C5)CC4C6)cc3)cc21 ; CC1(C)c2ccccc2-c2c(-c3ccc(C4CCCCC4)cc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)c(-c3ccccc3)cc2-c2c(-c3ccccc3)cccc21]

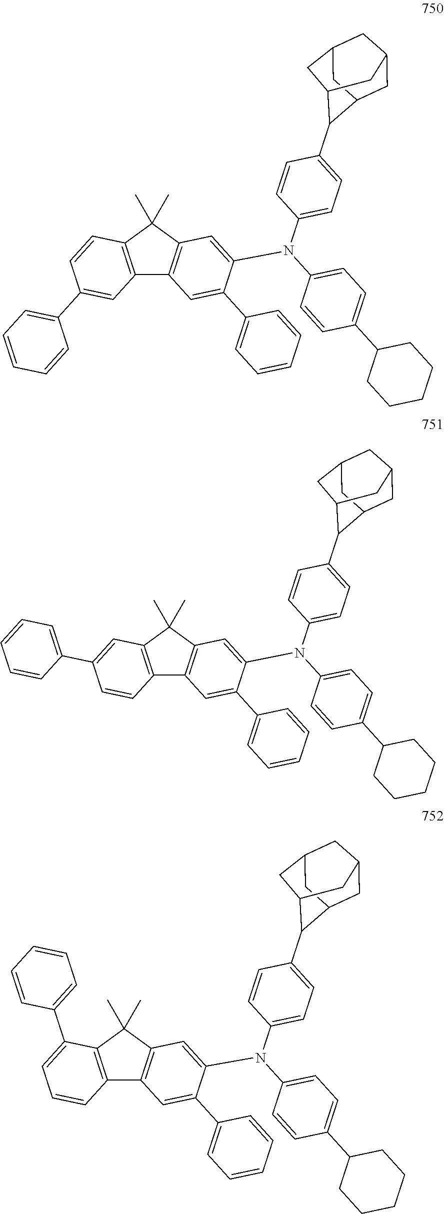 [SMILES: CC1(C)c2ccc(-c3ccccc3)cc2-c2cc(-c3ccccc3)c(N(c3ccc(C4CCCCC4)cc3)c3ccc(C4C5CC6CC(C5)CC4C6)cc3)cc21 ; CC1(C)c2cc(-c3ccccc3)ccc2-c2cc(-c3ccccc3)c(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)c(-c3ccccc3)cc2-c2cccc(-c3ccccc3)c21]

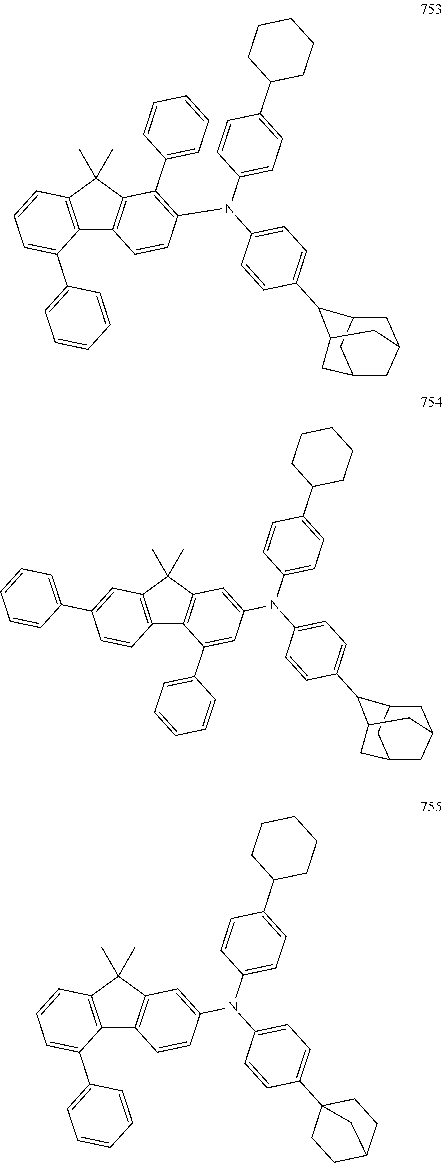 [SMILES: CC1(C)c2cccc(-c3ccccc3)c2-c2ccc(N(c3ccc(C4CCCCC4)cc3)c3ccc(C4C5CC6CC(C5)CC4C6)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C45CCC(CC4)C5)cc3)ccc2-c2c(-c3ccccc3)cccc21]

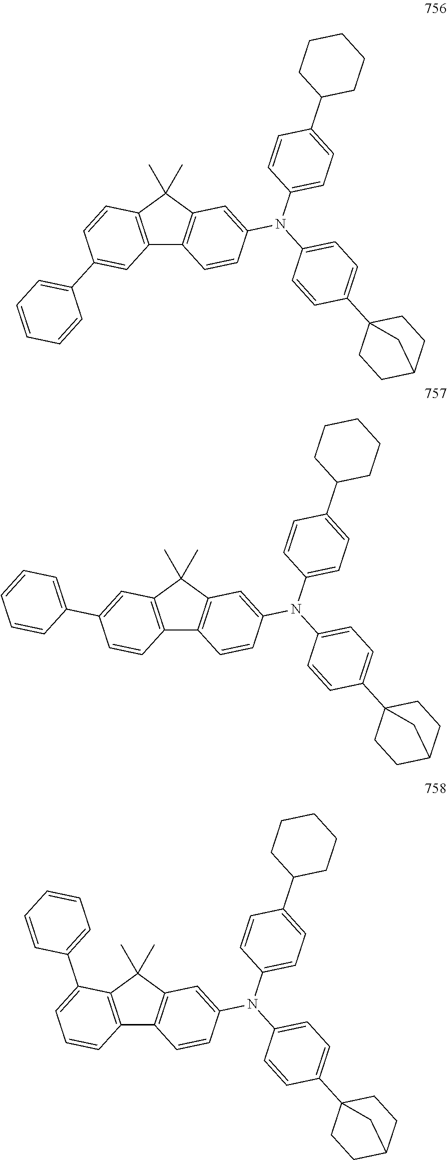

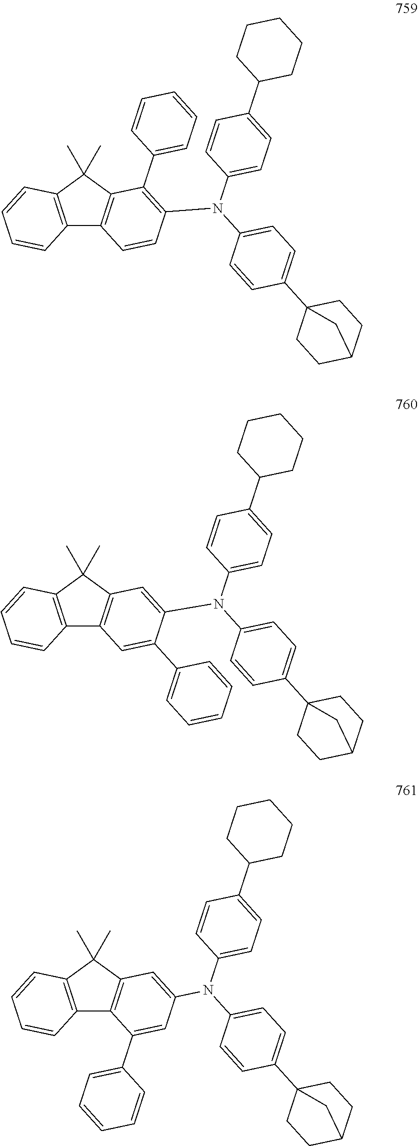 [SMILES: CC1(C)c2ccccc2-c2ccc(N(c3ccc(C4CCCCC4)cc3)c3ccc(C45CCC(CC4)C5)cc3)c(-c3ccccc3)c21 ; CC1(C)c2ccccc2-c2cc(-c3ccccc3)c(N(c3ccc(C4CCCCC4)cc3)c3ccc(C45CCC(CC4)C5)cc3)cc21 ; CC1(C)c2ccccc2-c2c(-c3ccccc3)cc(N(c3ccc(C4CCCCC4)cc3)c3ccc(C45CCC(CC4)C5)cc3)cc21]

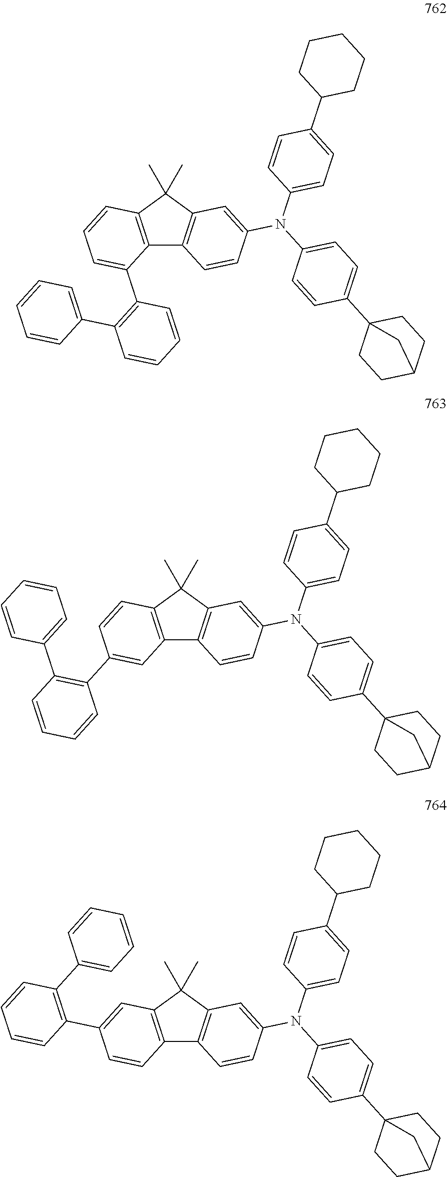 [SMILES: CC1(C)c2cc(N(c3ccc(C4CCCCC4)cc3)c3ccc(C45CCC(CC4)C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C45CCC(CC4)C5)cc3)cc21 ; CC1(C)c2cc(-c3ccccc3-c3ccccc3)ccc2-c2ccc(N(c3ccc(C4CCCCC4)cc3)c3ccc(C45CCC(CC4)C5)cc3)cc21]

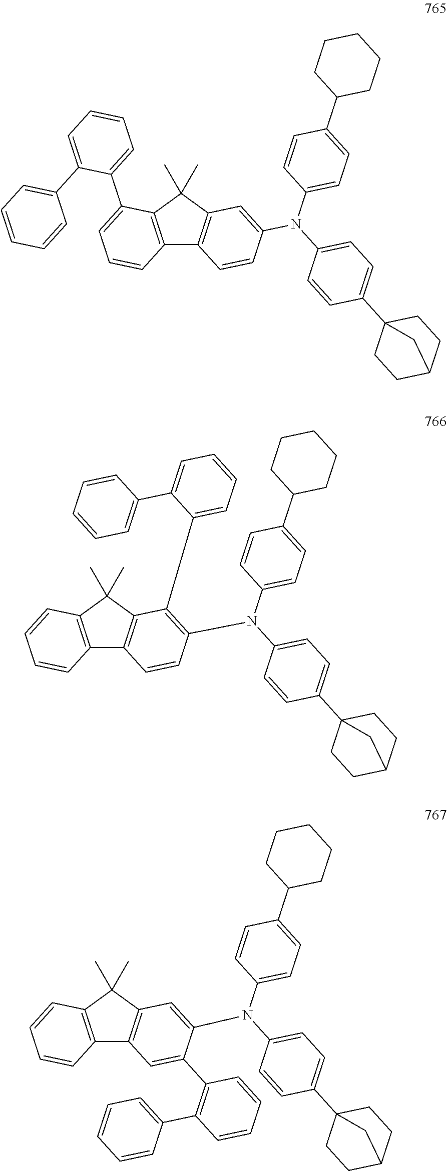 [SMILES: CC1(C)c2cc(N(c3ccc(C4CCCCC4)cc3)c3ccc(C45CCC(CC4)C5)cc3)ccc2-c2cccc(-c3ccccc3-c3ccccc3)c21 ; CC1(C)c2ccccc2-c2ccc(N(c3ccc(C4CCCCC4)cc3)c3ccc(C45CCC(CC4)C5)cc3)c(-c3ccccc3-c3ccccc3)c21 ; CC1(C)c2ccccc2-c2cc(-c3ccccc3-c3ccccc3)c(N(c3ccc(C4CCCCC4)cc3)c3ccc(C45CCC(CC4)C5)cc3)cc21]

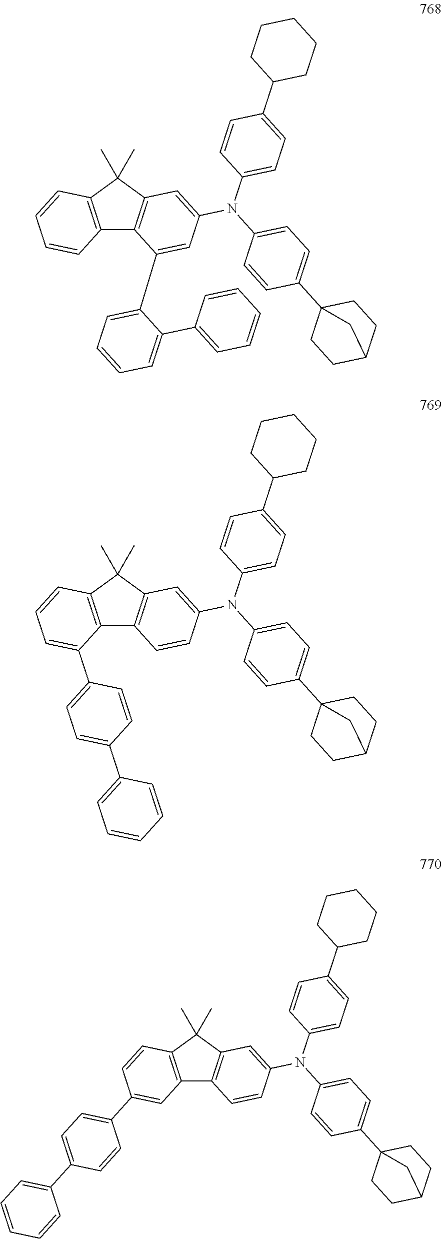 [SMILES: CC1(C)c2ccccc2-c2c(-c3ccccc3-c3ccccc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C45CCC(CC4)C5)cc3)cc21]

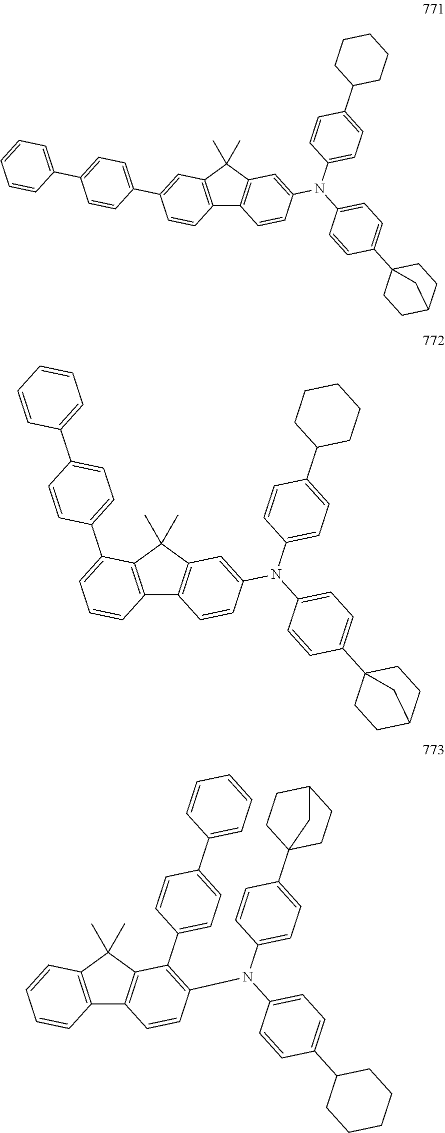 [SMILES: CC1(C)c2cc(-c3ccc(-c4ccccc4)cc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc(-c4ccccc4)cc3)c21 ; CC1(C)c2ccccc2-c2ccc(N(c3ccc(C4CCCCC4)cc3)c3ccc(C45CCC(CC4)C5)cc3)c(-c3ccc(-c4ccccc4)cc3)c21]

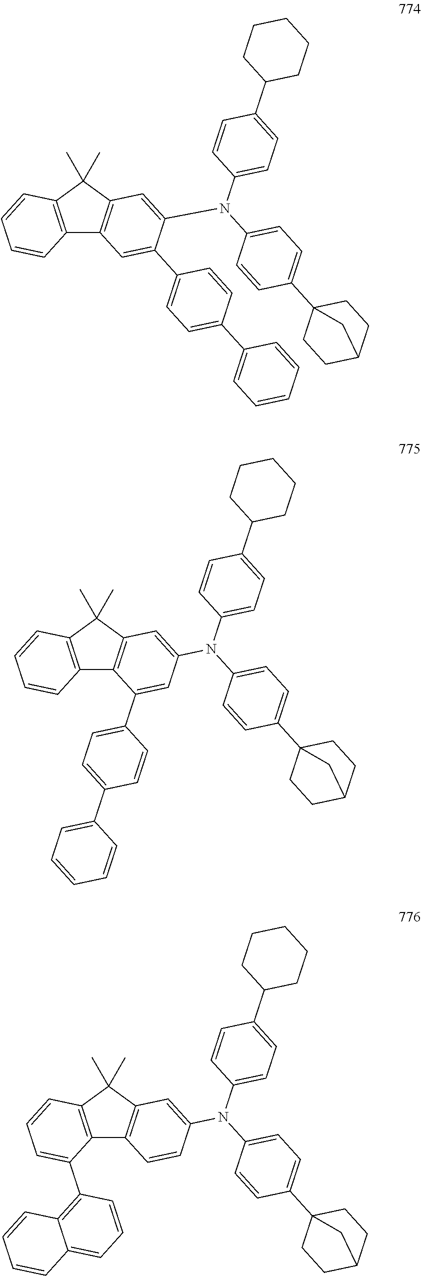 [SMILES: CC1(C)c2ccccc2-c2cc(-c3ccc(-c4ccccc4)cc3)c(N(c3ccc(C4CCCCC4)cc3)c3ccc(C45CCC(CC4)C5)cc3)cc21 ; CC1(C)c2ccccc2-c2c(-c3ccc(-c4ccccc4)cc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3cccc4ccccc34)cccc21]

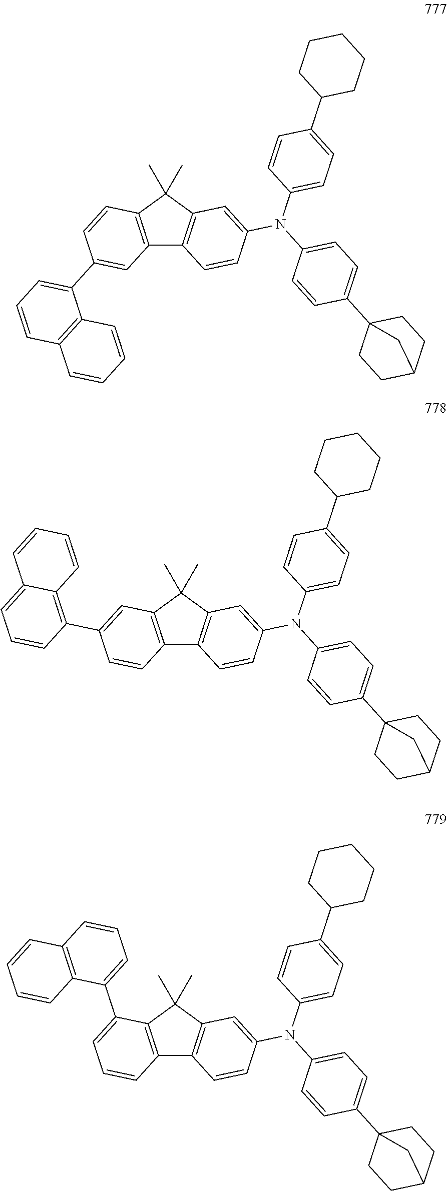 [SMILES: CC1(C)c2ccc(-c3cccc4ccccc34)cc2-c2ccc(N(c3ccc(C4CCCCC4)cc3)c3ccc(C45CCC(CC4)C5)cc3)cc21 ; CC1(C)c2cc(-c3cccc4ccccc34)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3cccc4ccccc34)c21]

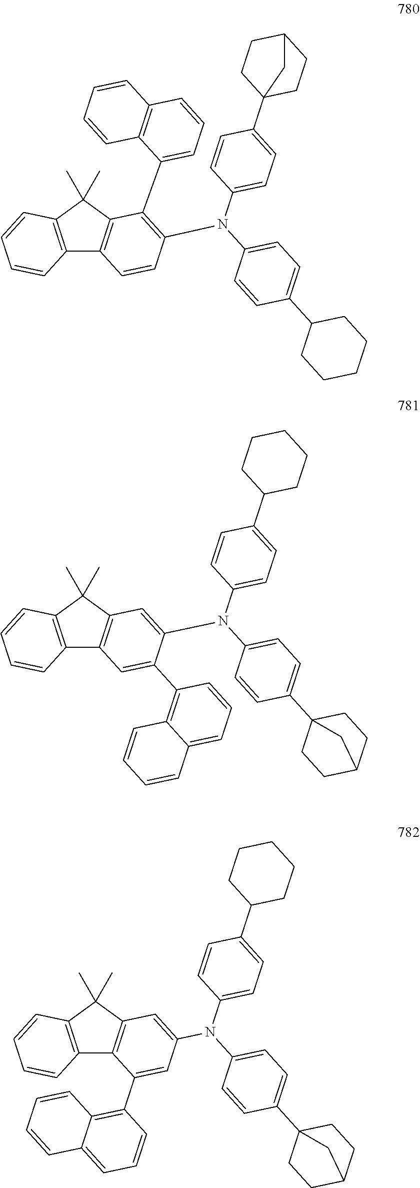 [SMILES: CC1(C)c2ccccc2-c2ccc(N(c3ccc(C4CCCCC4)cc3)c3ccc(C45CCC(CC4)C5)cc3)c(-c3cccc4ccccc34)c21 ; CC1(C)c2ccccc2-c2cc(-c3cccc4ccccc34)c(N(c3ccc(C4CCCCC4)cc3)c3ccc(C45CCC(CC4)C5)cc3)cc21 ; CC1(C)c2ccccc2-c2c(-c3cccc4ccccc34)cc(N(c3ccc(C4CCCCC4)cc3)c3ccc(C45CCC(CC4)C5)cc3)cc21]

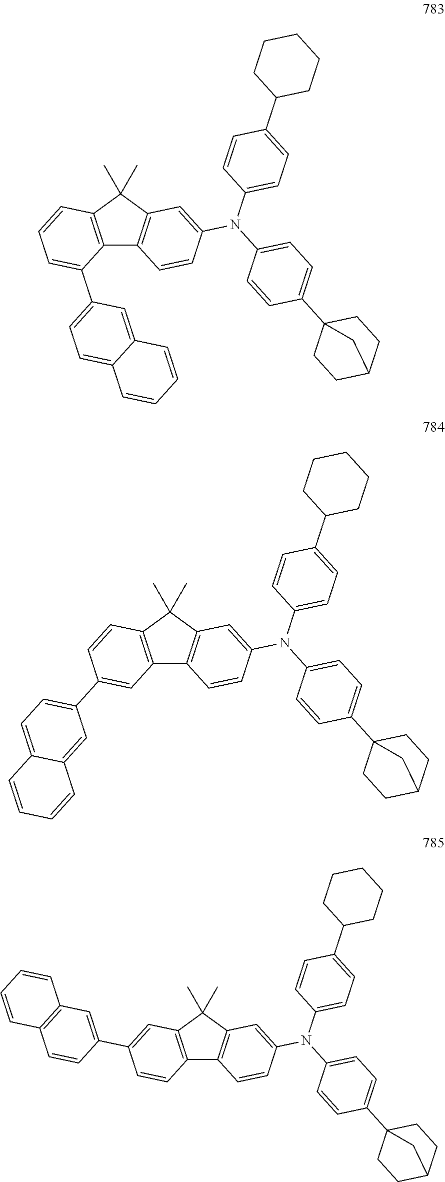 [SMILES: CC1(C)c2cc(N(c3ccc(C4CCCCC4)cc3)c3ccc(C45CCC(CC4)C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C45CCC(CC4)C5)cc3)cc21 ; CC1(C)c2cc(-c3ccc4ccccc4c3)ccc2-c2ccc(N(c3ccc(C4CCCCC4)cc3)c3ccc(C45CCC(CC4)C5)cc3)cc21]

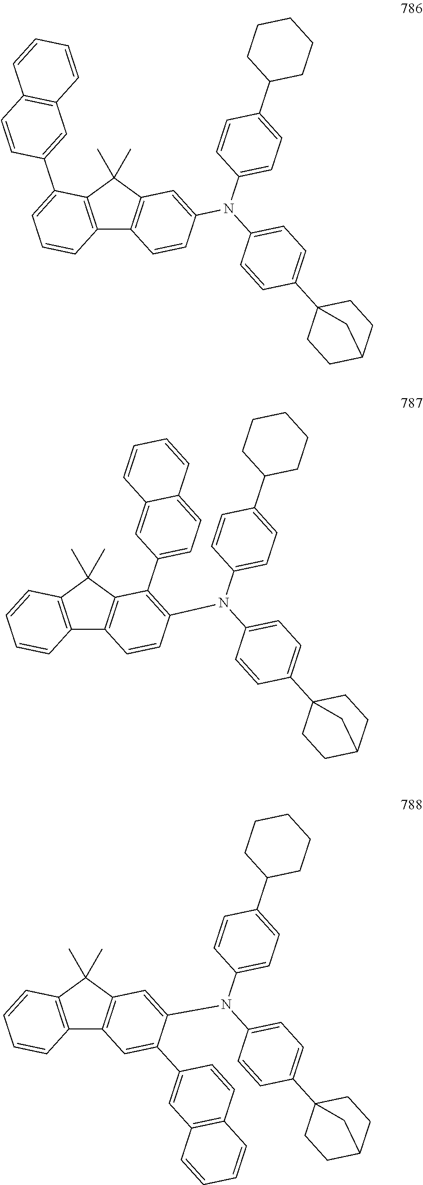 [SMILES: CC1(C)c2cc(N(c3ccc(C4CCCCC4)cc3)c3ccc(C45CCC(CC4)C5)cc3)ccc2-c2cccc(-c3ccc4ccccc4c3)c21 ; CC1(C)c2ccccc2-c2ccc(N(c3ccc(C4CCCCC4)cc3)c3ccc(C45CCC(CC4)C5)cc3)c(-c3ccc4ccccc4c3)c21 ; CC1(C)c2ccccc2-c2cc(-c3ccc4ccccc4c3)c(N(c3ccc(C4CCCCC4)cc3)c3ccc(C45CCC(CC4)C5)cc3)cc21]

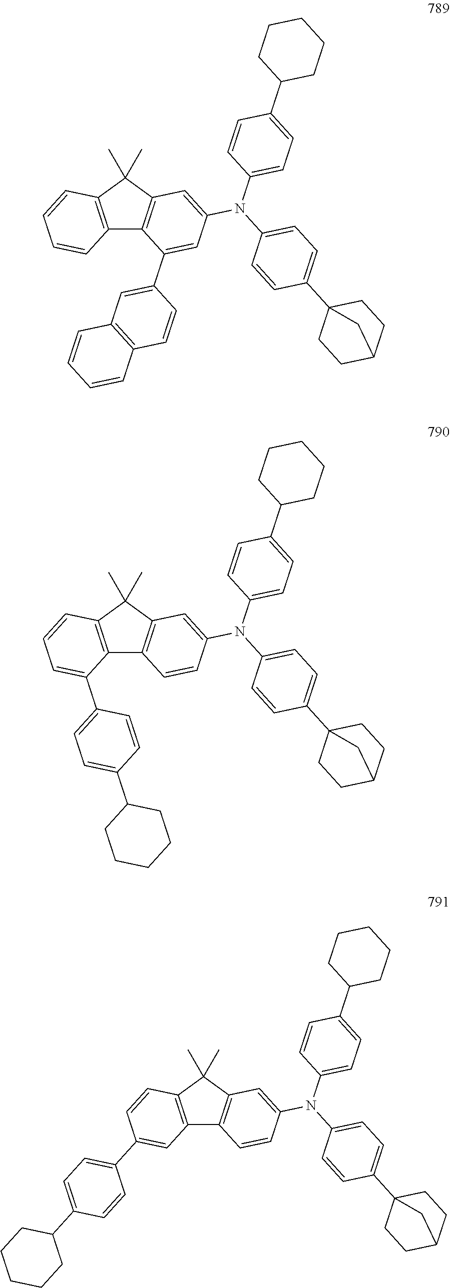 [SMILES: CC1(C)c2ccccc2-c2c(-c3ccc4ccccc4c3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CCCCC4)cc3)c3ccc(C45CCC(CC4)C5)cc3)cc21]

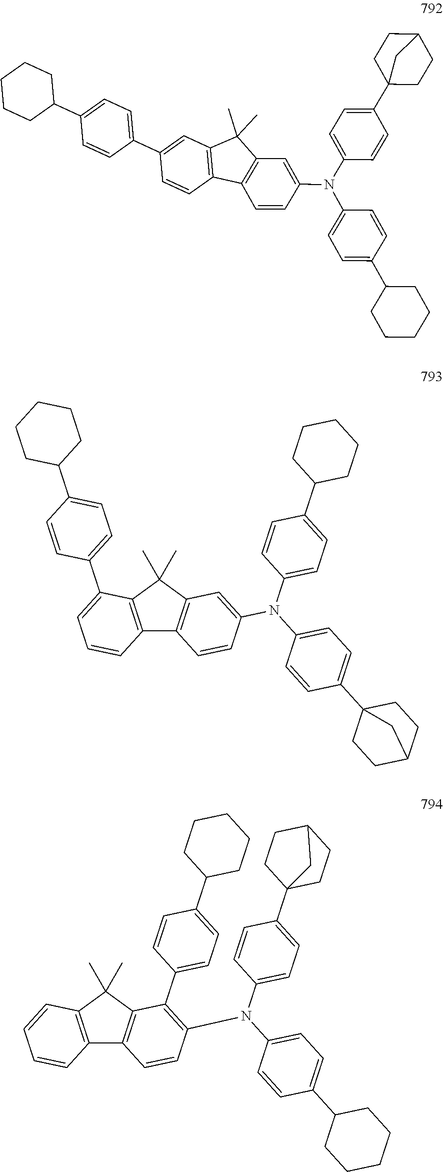 [SMILES: CC1(C)c2cc(-c3ccc(C4CCCCC4)cc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc(C4CCCCC4)cc3)c21 ; CC1(C)c2ccccc2-c2ccc(N(c3ccc(C4CCCCC4)cc3)c3ccc(C45CCC(CC4)C5)cc3)c(-c3ccc(C4CCCCC4)cc3)c21]

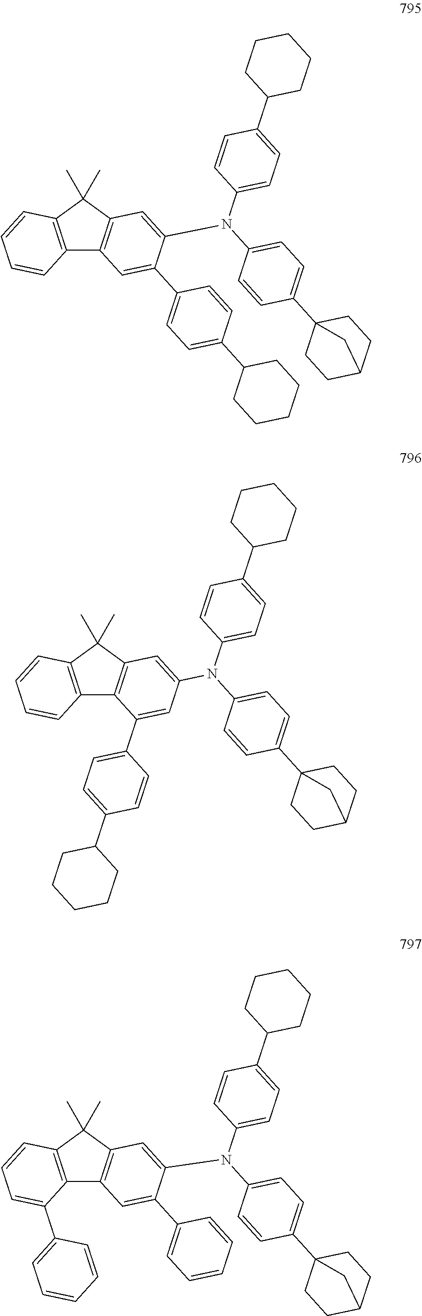 [SMILES: CC1(C)c2ccccc2-c2cc(-c3ccc(C4CCCCC4)cc3)c(N(c3ccc(C4CCCCC4)cc3)c3ccc(C45CCC(CC4)C5)cc3)cc21 ; CC1(C)c2ccccc2-c2c(-c3ccc(C4CCCCC4)cc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)c(-c3ccccc3)cc2-c2c(-c3ccccc3)cccc21]

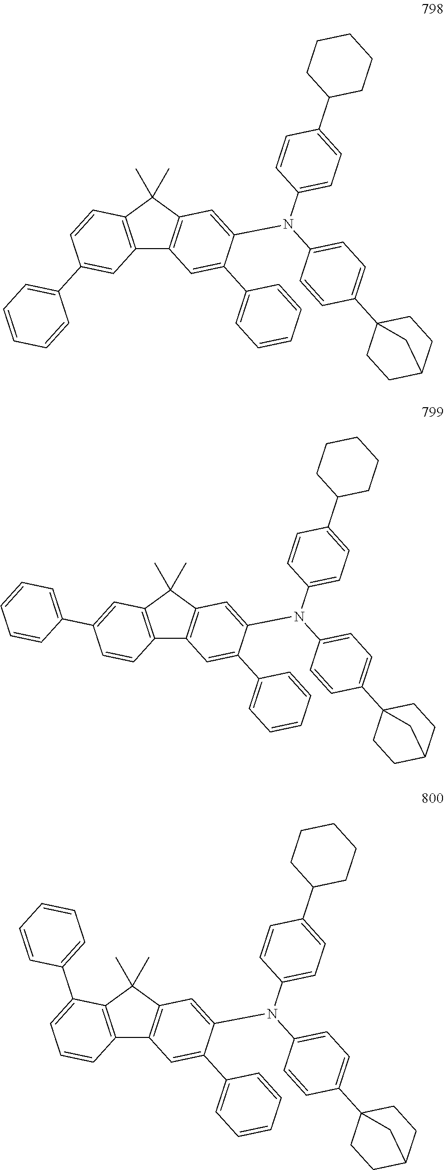 [SMILES: CC1(C)c2ccc(-c3ccccc3)cc2-c2cc(-c3ccccc3)c(N(c3ccc(C4CCCCC4)cc3)c3ccc(C45CCC(CC4)C5)cc3)cc21 ; CC1(C)c2cc(-c3ccccc3)ccc2-c2cc(-c3ccccc3)c(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)c(-c3ccccc3)cc2-c2cccc(-c3ccccc3)c21]

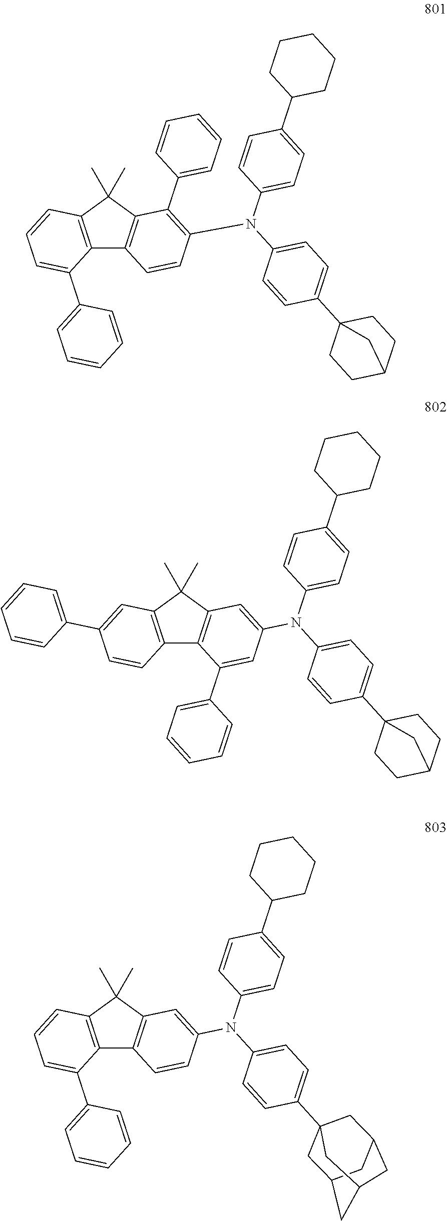 [SMILES: CC1(C)c2cccc(-c3ccccc3)c2-c2ccc(N(c3ccc(C4CCCCC4)cc3)c3ccc(C45CCC(CC4)C5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccccc3)cccc21]

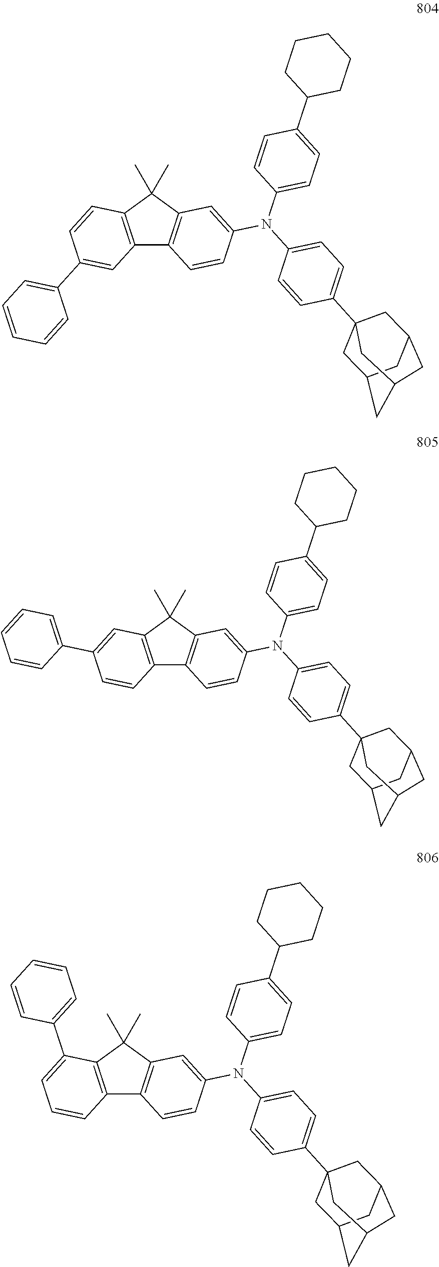 [SMILES: CC1(C)c2ccc(-c3ccccc3)cc2-c2ccc(N(c3ccc(C4CCCCC4)cc3)c3ccc(C45CC6CC(CC(C6)C4)C5)cc3)cc21 ; CC1(C)c2cc(-c3ccccc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccccc3)c21]

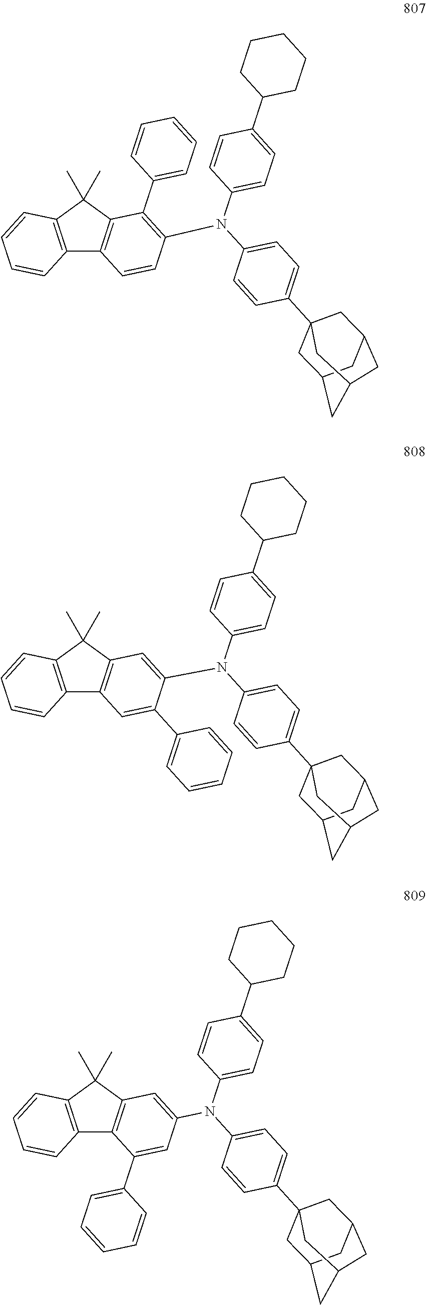 [SMILES: CC1(C)c2ccccc2-c2ccc(N(c3ccc(C4CCCCC4)cc3)c3ccc(C45CC6CC(CC(C6)C4)C5)cc3)c(-c3ccccc3)c21 ; CC1(C)c2ccccc2-c2cc(-c3ccccc3)c(N(c3ccc(C4CCCCC4)cc3)c3ccc(C45CC6CC(CC(C6)C4)C5)cc3)cc21 ; CC1(C)c2ccccc2-c2c(-c3ccccc3)cc(N(c3ccc(C4CCCCC4)cc3)c3ccc(C45CC6CC(CC(C6)C4)C5)cc3)cc21]

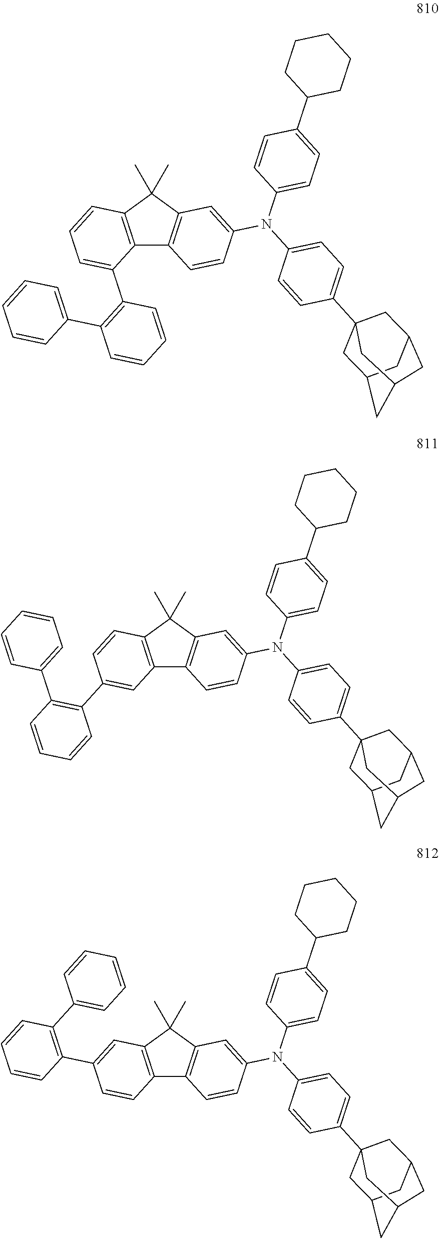 [SMILES: CC1(C)c2cc(N(c3ccc(C4CCCCC4)cc3)c3ccc(C45CC6CC(CC(C6)C4)C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C45CC6CC(CC(C6)C4)C5)cc3)cc21 ; CC1(C)c2cc(-c3ccccc3-c3ccccc3)ccc2-c2ccc(N(c3ccc(C4CCCCC4)cc3)c3ccc(C45CC6CC(CC(C6)C4)C5)cc3)cc21]

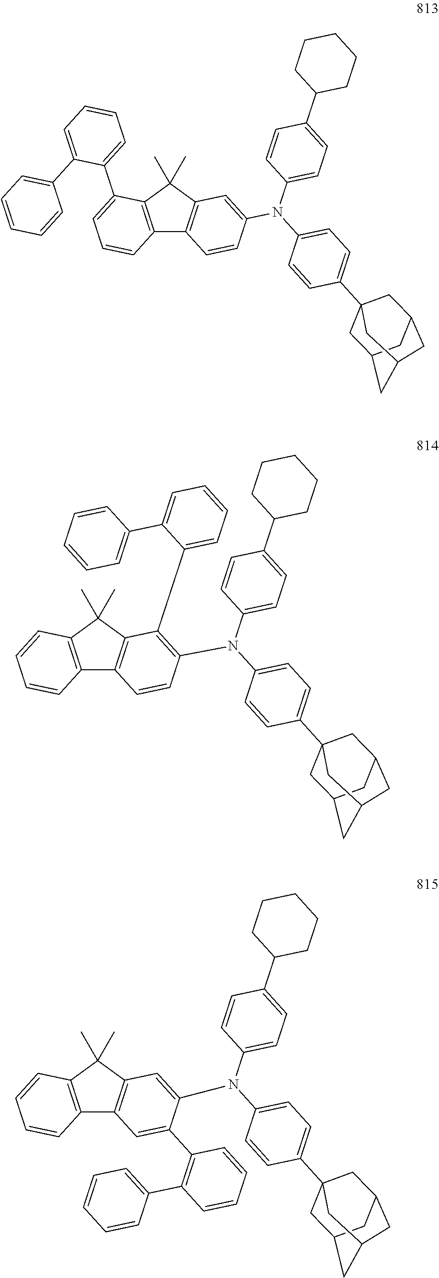 [SMILES: CC1(C)c2cc(N(c3ccc(C4CCCCC4)cc3)c3ccc(C45CC6CC(CC(C6)C4)C5)cc3)ccc2-c2cccc(-c3ccccc3-c3ccccc3)c21 ; CC1(C)c2ccccc2-c2ccc(N(c3ccc(C4CCCCC4)cc3)c3ccc(C45CC6CC(CC(C6)C4)C5)cc3)c(-c3ccccc3-c3ccccc3)c21 ; CC1(C)c2ccccc2-c2cc(-c3ccccc3-c3ccccc3)c(N(c3ccc(C4CCCCC4)cc3)c3ccc(C45CC6CC(CC(C6)C4)C5)cc3)cc21]

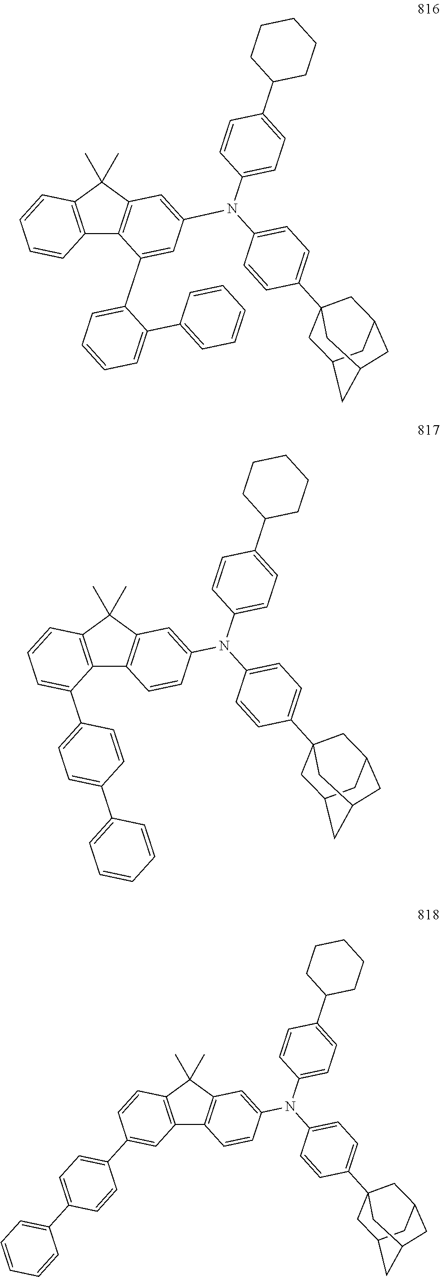 [SMILES: CC1(C)c2ccccc2-c2c(-c3ccccc3-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C45CC6CC(CC(C6)C4)C5)cc3)cc21]

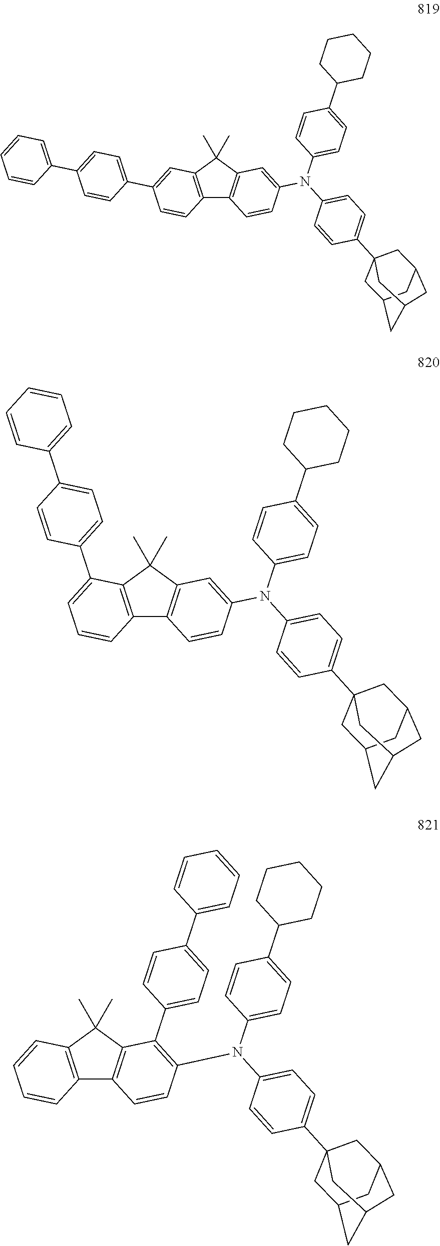 [SMILES: CC1(C)c2cc(-c3ccc(-c4ccccc4)cc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc(-c4ccccc4)cc3)c21 ; CC1(C)c2ccccc2-c2ccc(N(c3ccc(C4CCCCC4)cc3)c3ccc(C45CC6CC(CC(C6)C4)C5)cc3)c(-c3ccc(-c4ccccc4)cc3)c21]

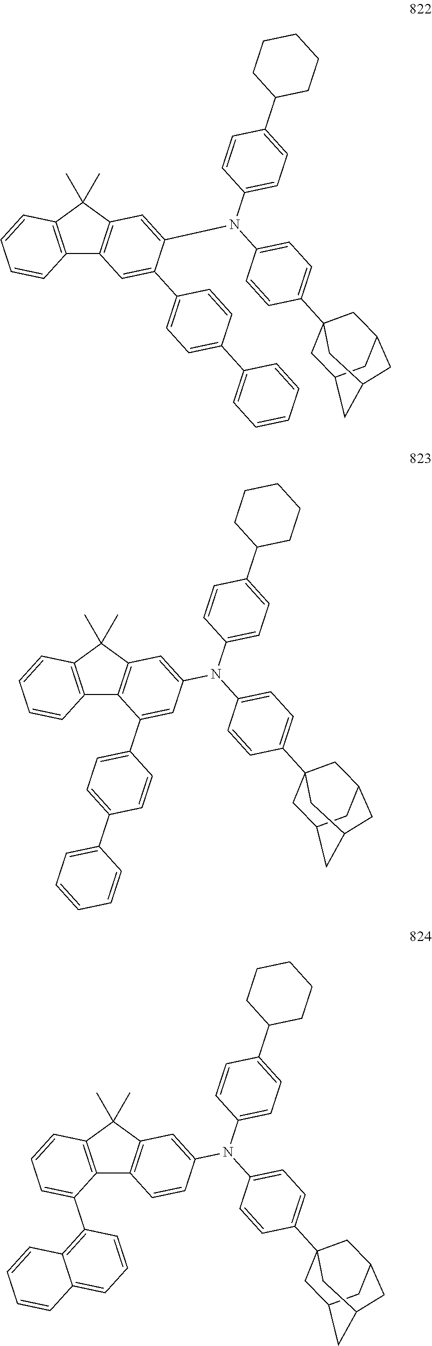 [SMILES: CC1(C)c2ccccc2-c2cc(-c3ccc(-c4ccccc4)cc3)c(N(c3ccc(C4CCCCC4)cc3)c3ccc(C45CC6CC(CC(C6)C4)C5)cc3)cc21 ; CC1(C)c2ccccc2-c2c(-c3ccc(-c4ccccc4)cc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3cccc4ccccc34)cccc21]

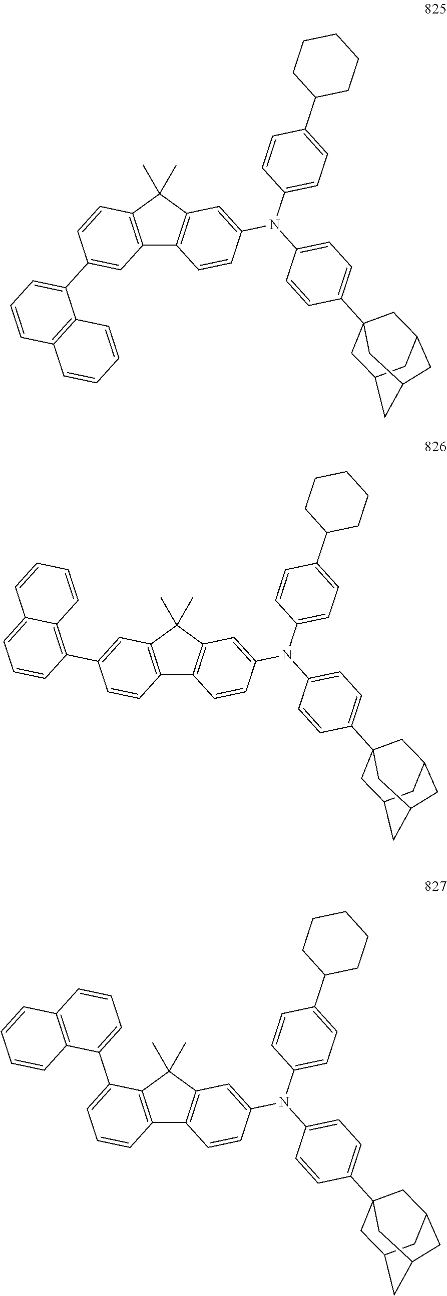 [SMILES: CC1(C)c2ccc(-c3cccc4ccccc34)cc2-c2ccc(N(c3ccc(C4CCCCC4)cc3)c3ccc(C45CC6CC(CC(C6)C4)C5)cc3)cc21 ; CC1(C)c2cc(-c3cccc4ccccc34)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3cccc4ccccc34)c21]

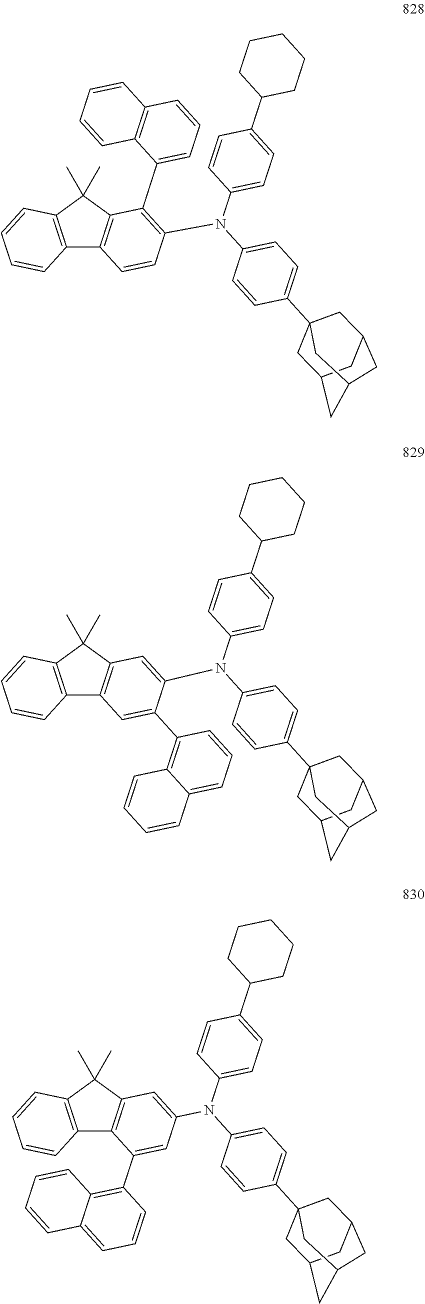 [SMILES: CC1(C)c2ccccc2-c2ccc(N(c3ccc(C4CCCCC4)cc3)c3ccc(C45CC6CC(CC(C6)C4)C5)cc3)c(-c3cccc4ccccc34)c21 ; CC1(C)c2ccccc2-c2cc(-c3cccc4ccccc34)c(N(c3ccc(C4CCCCC4)cc3)c3ccc(C45CC6CC(CC(C6)C4)C5)cc3)cc21 ; CC1(C)c2ccccc2-c2c(-c3cccc4ccccc34)cc(N(c3ccc(C4CCCCC4)cc3)c3ccc(C45CC6CC(CC(C6)C4)C5)cc3)cc21]

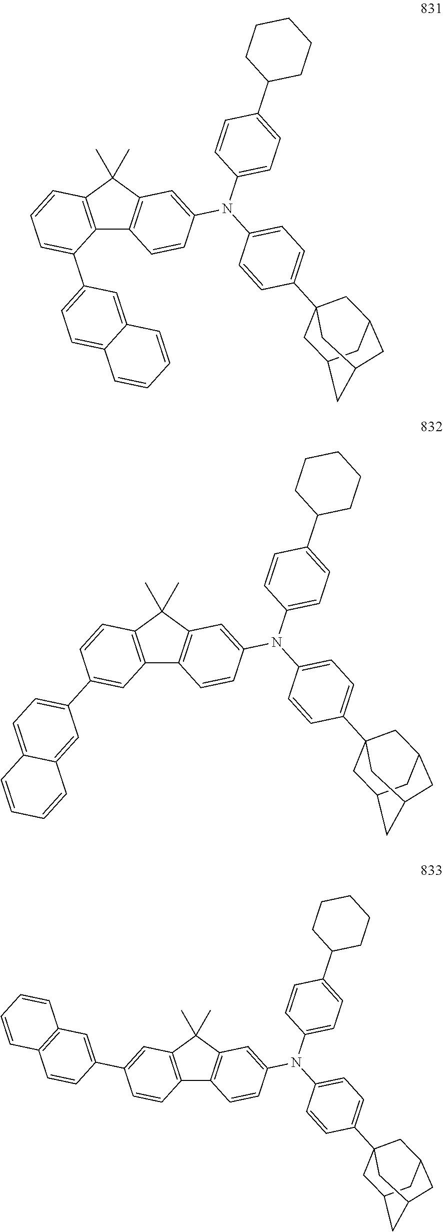 [SMILES: CC1(C)c2cc(N(c3ccc(C4CCCCC4)cc3)c3ccc(C45CC6CC(CC(C6)C4)C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C45CC6CC(CC(C6)C4)C5)cc3)cc21 ; CC1(C)c2cc(-c3ccc4ccccc4c3)ccc2-c2ccc(N(c3ccc(C4CCCCC4)cc3)c3ccc(C45CC6CC(CC(C6)C4)C5)cc3)cc21]

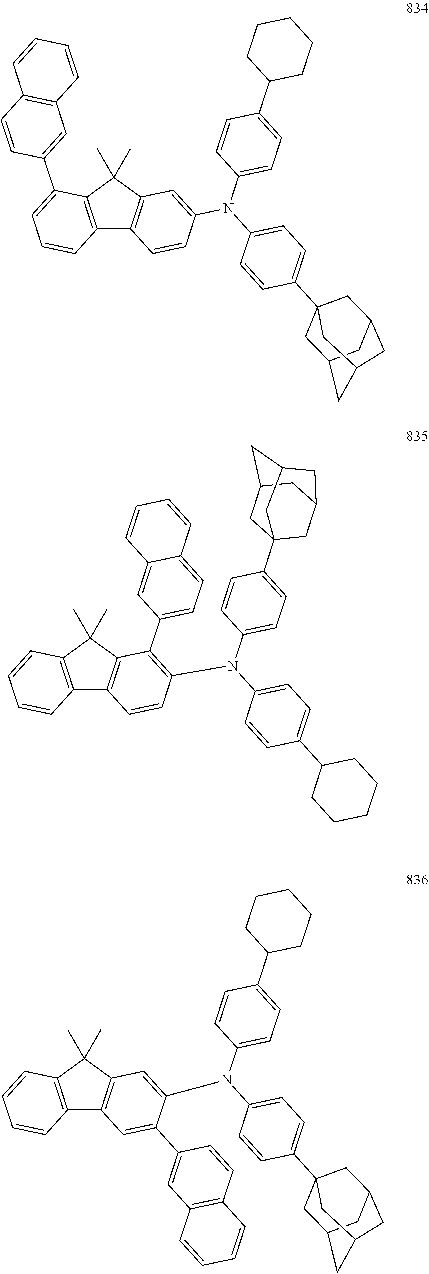 [SMILES: CC1(C)c2cc(N(c3ccc(C4CCCCC4)cc3)c3ccc(C45CC6CC(CC(C6)C4)C5)cc3)ccc2-c2cccc(-c3ccc4ccccc4c3)c21 ; CC1(C)c2ccccc2-c2ccc(N(c3ccc(C4CCCCC4)cc3)c3ccc(C45CC6CC(CC(C6)C4)C5)cc3)c(-c3ccc4ccccc4c3)c21 ; CC1(C)c2ccccc2-c2cc(-c3ccc4ccccc4c3)c(N(c3ccc(C4CCCCC4)cc3)c3ccc(C45CC6CC(CC(C6)C4)C5)cc3)cc21]

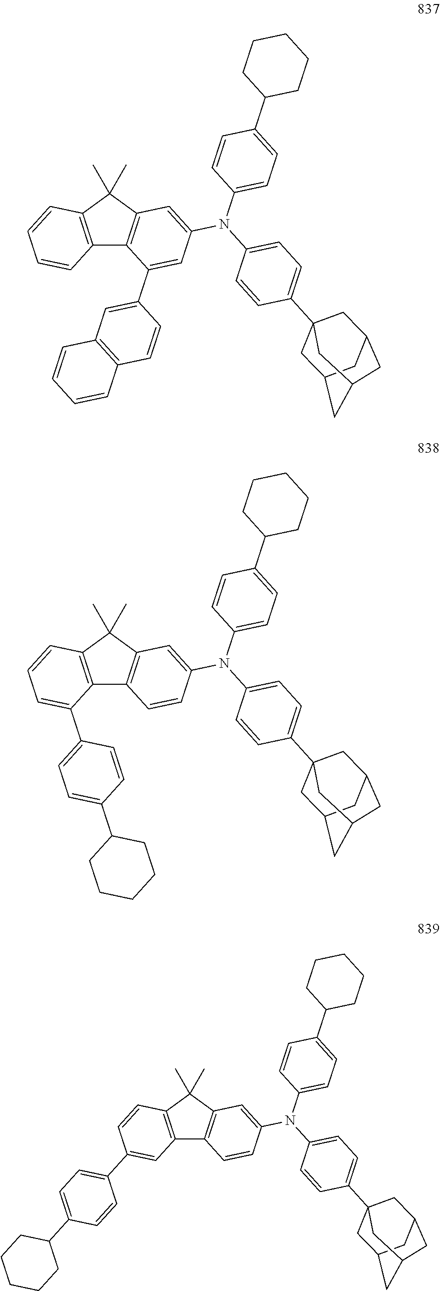 [SMILES: CC1(C)c2ccccc2-c2c(-c3ccc4ccccc4c3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(C4CCCCC4)cc3)c3ccc(C45CC6CC(CC(C6)C4)C5)cc3)cc21]

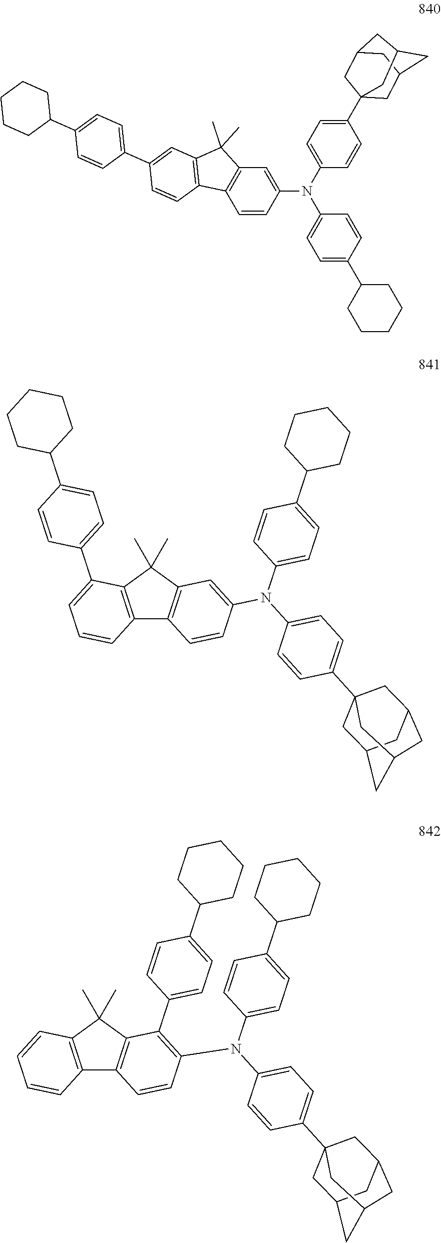 [SMILES: CC1(C)c2cc(-c3ccc(C4CCCCC4)cc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc(C4CCCCC4)cc3)c21 ; CC1(C)c2ccccc2-c2ccc(N(c3ccc(C4CCCCC4)cc3)c3ccc(C45CC6CC(CC(C6)C4)C5)cc3)c(-c3ccc(C4CCCCC4)cc3)c21]

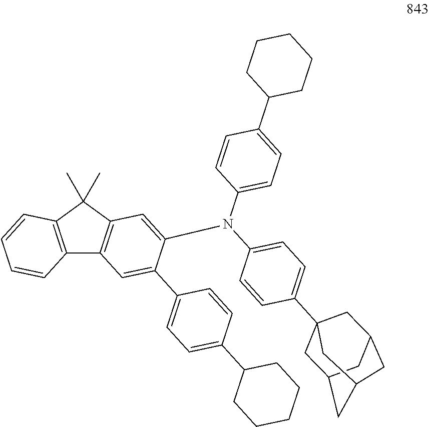 [SMILES: CC1(C)c2ccccc2-c2cc(-c3ccc(C4CCCCC4)cc3)c(N(c3ccc(C4CCCCC4)cc3)c3ccc(C45CC6CC(CC(C6)C4)C5)cc3)cc21]

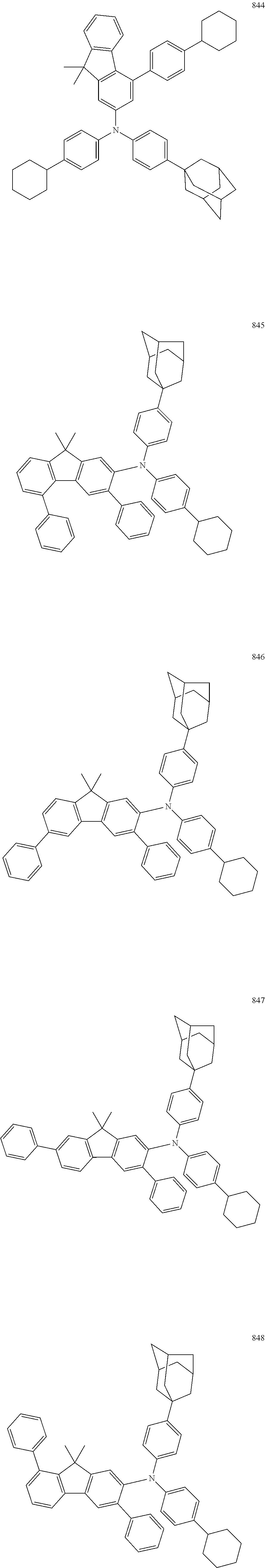

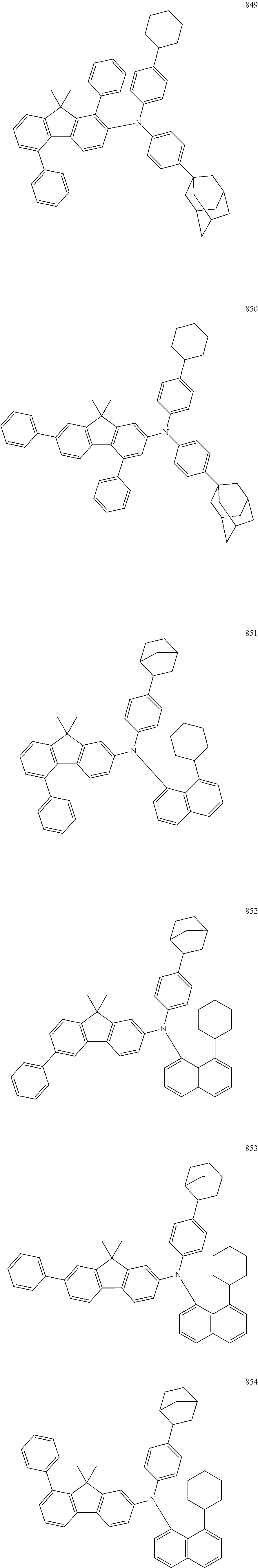

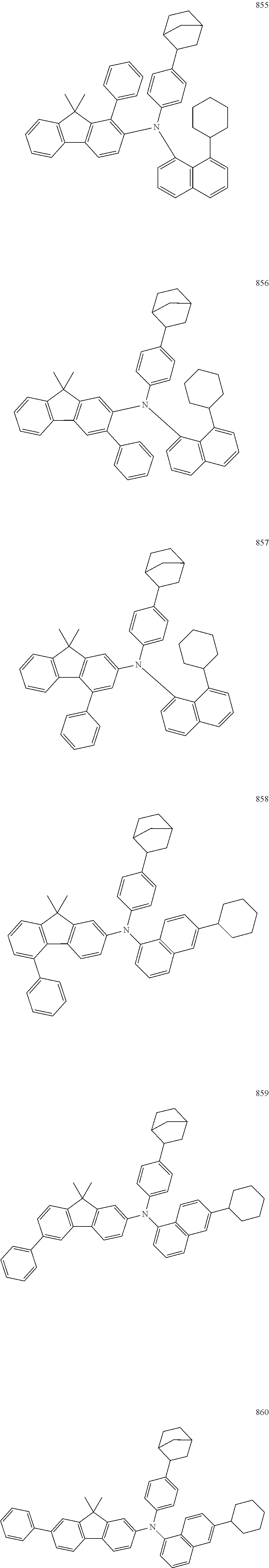

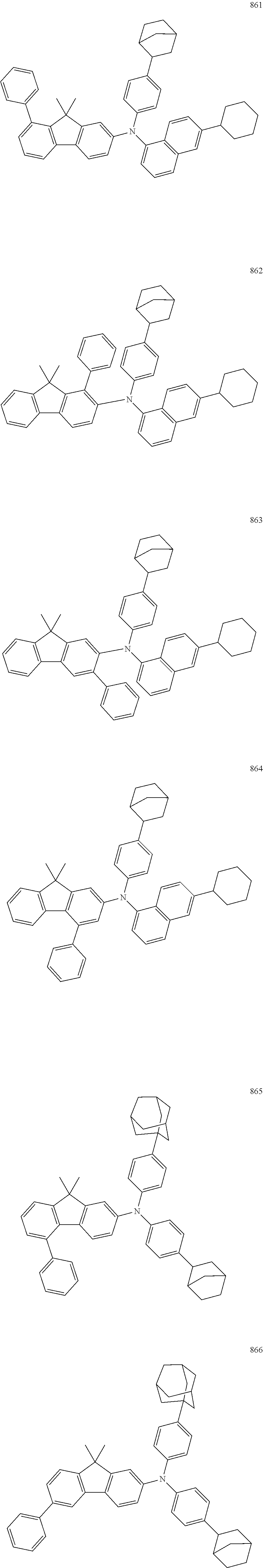

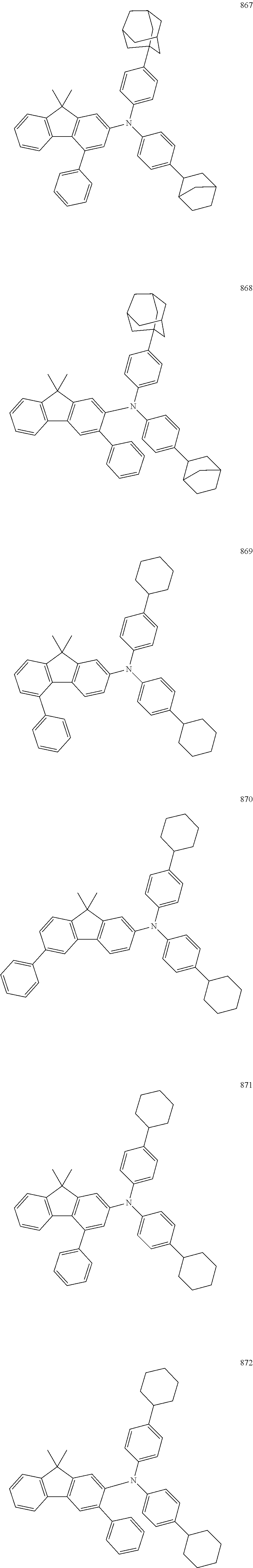

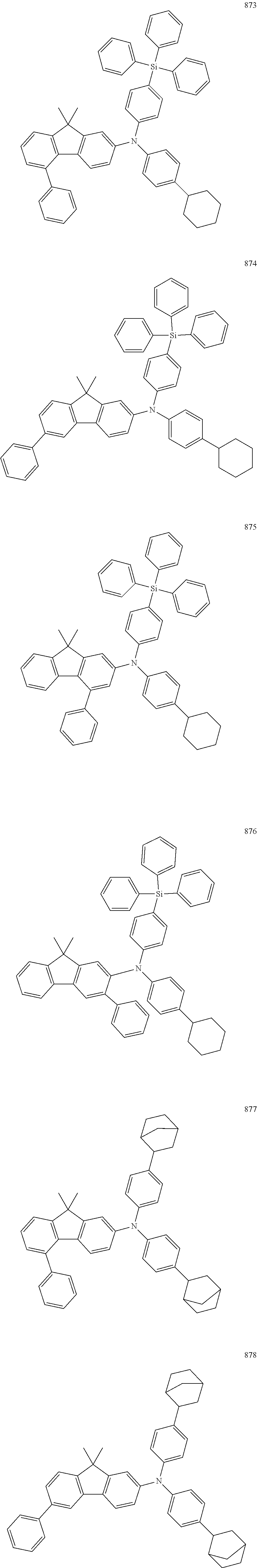

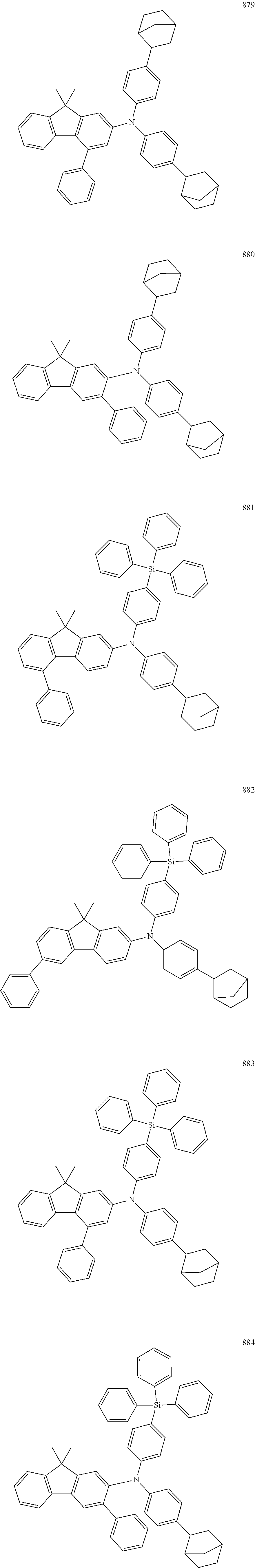

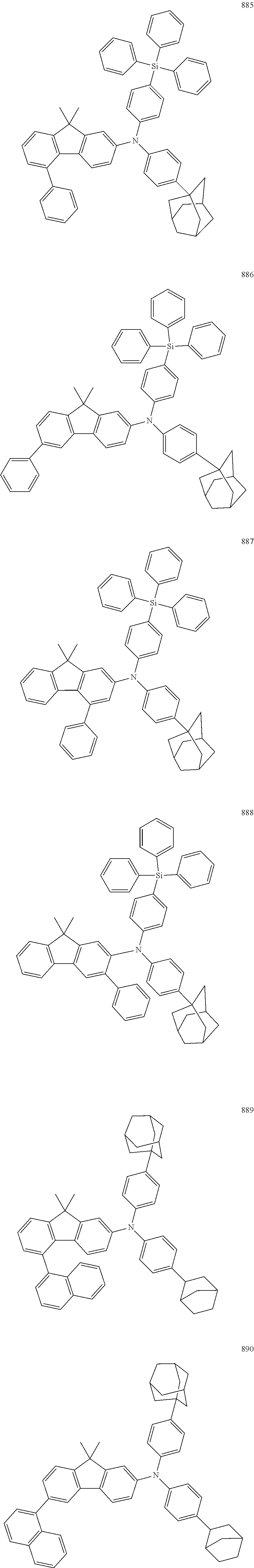

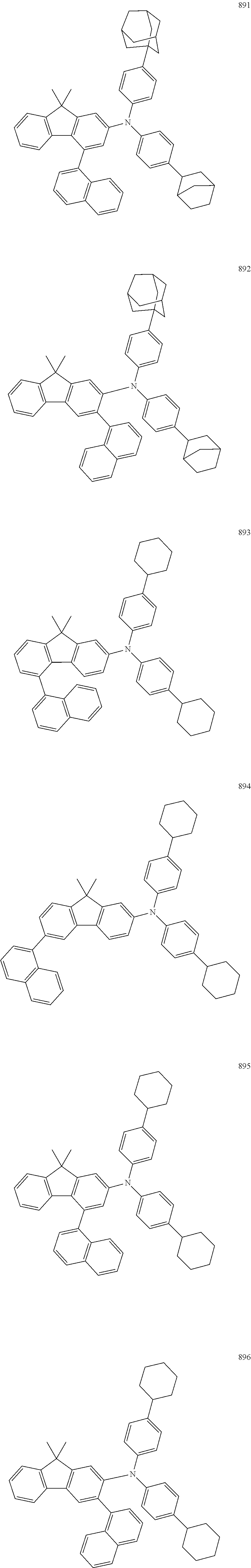

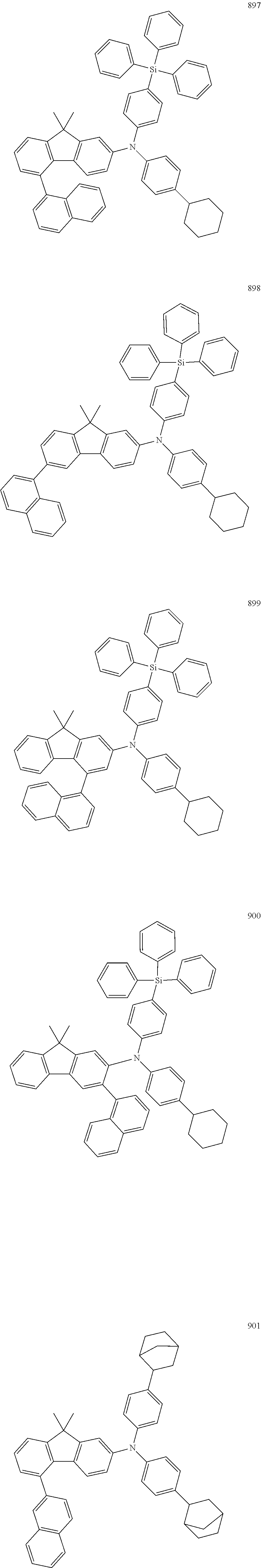

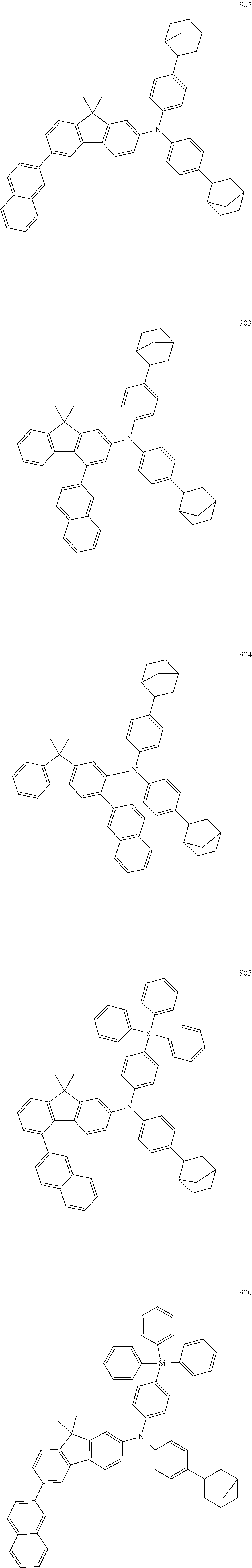 [SMILES: CC1(C)c2ccc(-c3ccc4ccccc4c3)cc2-c2ccc(N(c3ccc(C4CC5CCC4C5)cc3)c3ccc(C4CC5CCC4C5)cc3)cc21 ; CC1(C)c2ccccc2-c2c(-c3ccc4ccccc4c3)cc(N(c3ccc(C4CC5CCC4C5)cc3)c3ccc(C4CC5CCC4C5)cc3)cc21 ; CC1(C)c2ccccc2-c2cc(-c3ccc4ccccc4c3)c(N(c3ccc(C4CC5CCC4C5)cc3)c3ccc(C4CC5CCC4C5)cc3)cc21 ; CC1(C)c2cc(N(c3ccc(C4CC5CCC4C5)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccc([Si](c4ccccc4)(c4ccccc4)c4ccccc4)cc3)cc21]

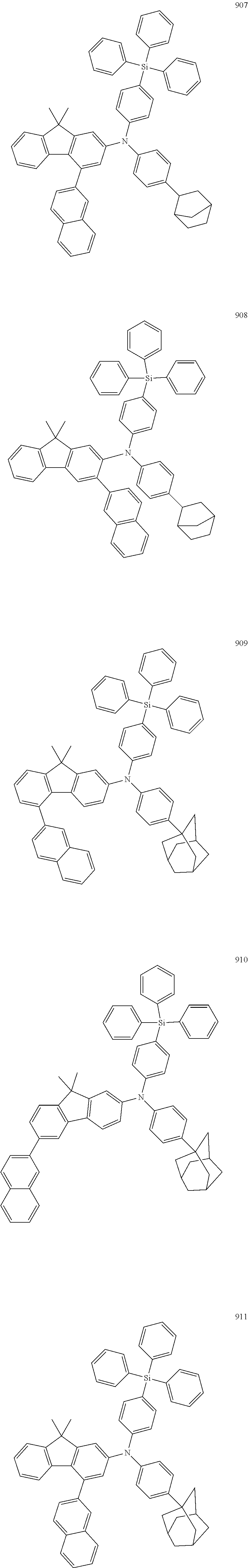

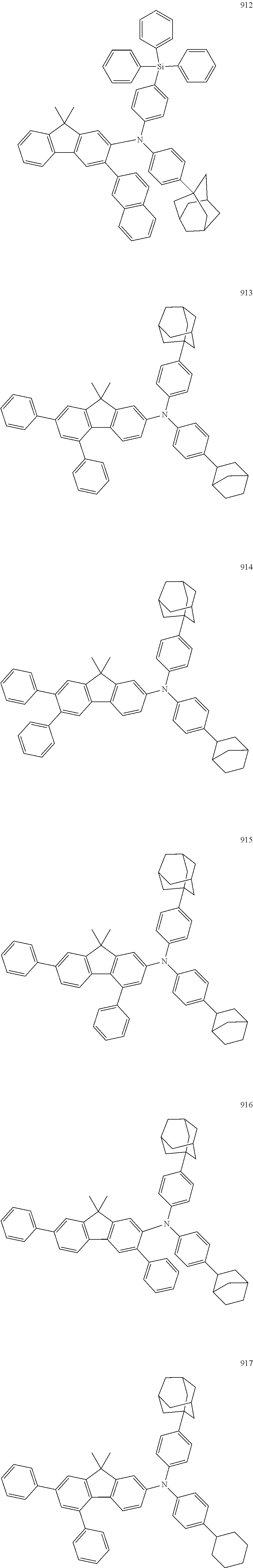

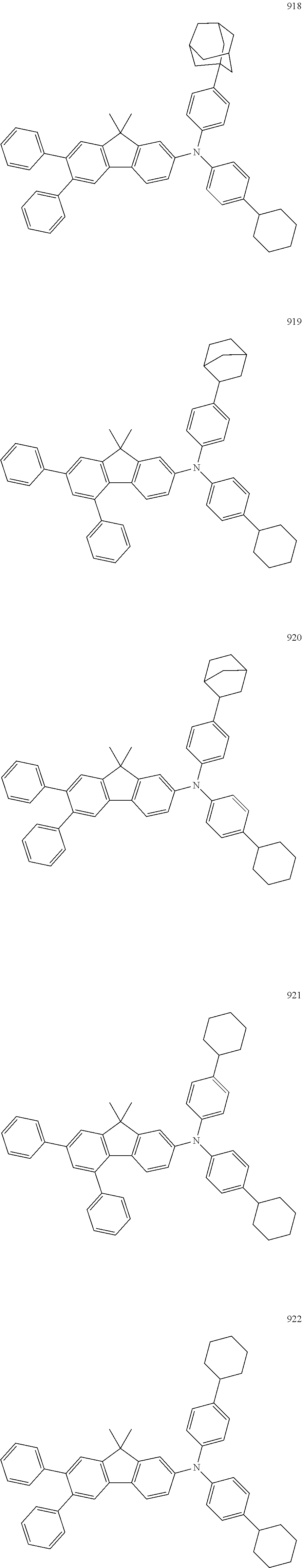 [SMILES: CC1(C)c2cc(N(c3ccc(C4CCCCC4)cc3)c3ccc(C45CC6CC(CC(C6)C4)C5)cc3)ccc2-c2cc(-c3ccccc3)c(-c3ccccc3)cc21 ; CC1(C)c2cc(N(c3ccc(C4CCCCC4)cc3)c3ccc(C4CC5CCC4C5)cc3)ccc2-c2c(-c3ccccc3)cc(-c3ccccc3)cc21 ; CC1(C)c2cc(N(c3ccc(C4CCCCC4)cc3)c3ccc(C4CC5CCC4C5)cc3)ccc2-c2cc(-c3ccccc3)c(-c3ccccc3)cc21 ; CC1(C)c2cc(N(c3ccc(C4CCCCC4)cc3)c3ccc(C4CCCCC4)cc3)ccc2-c2c(-c3ccccc3)cc(-c3ccccc3)cc21 ; CC1(C)c2cc(N(c3ccc(C4CCCCC4)cc3)c3ccc(C4CCCCC4)cc3)ccc2-c2cc(-c3ccccc3)c(-c3ccccc3)cc21]

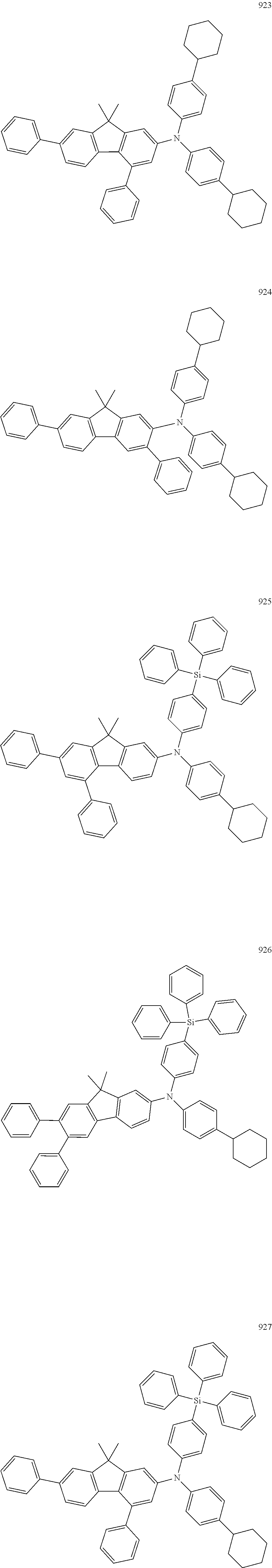

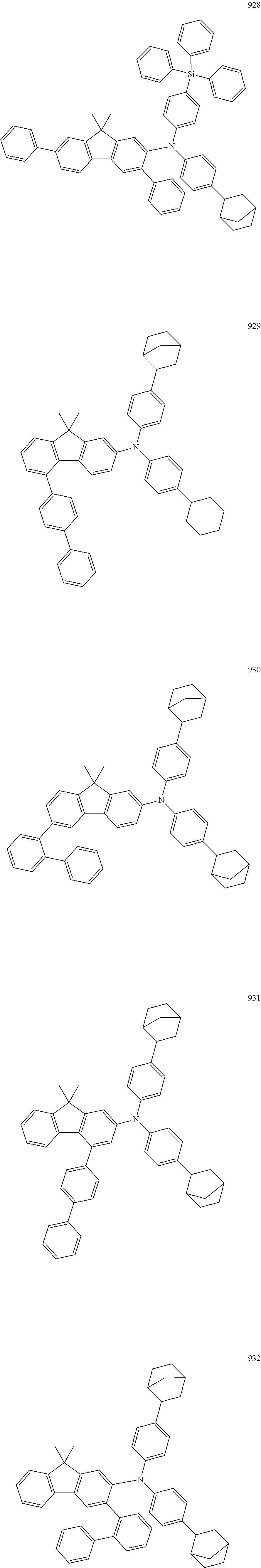 [SMILES: CC1(C)c2cc(-c3ccccc3)ccc2-c2cc(-c3ccccc3)c(N(c3ccc(C4CC5CCC4C5)cc3)c3ccc([Si](c4ccccc4)(c4ccccc4)c4ccccc4)cc3)cc21 ; CC1(C)c2cc(N(c3ccc(C4CCCCC4)cc3)c3ccc(C4CC5CCC4C5)cc3)ccc2-c2c(-c3ccc(-c4ccccc4)cc3)cccc21 ; CC1(C)c2ccc(-c3ccccc3-c3ccccc3)cc2-c2ccc(N(c3ccc(C4CC5CCC4C5)cc3)c3ccc(C4CC5CCC4C5)cc3)cc21 ; CC1(C)c2ccccc2-c2c(-c3ccc(-c4ccccc4)cc3)cc(N(c3ccc(C4CC5CCC4C5)cc3)c3ccc(C4CC5CCC4C5)cc3)cc21 ; CC1(C)c2ccccc2-c2cc(-c3ccccc3-c3ccccc3)c(N(c3ccc(C4CC5CCC4C5)cc3)c3ccc(C4CC5CCC4C5)cc3)cc21]

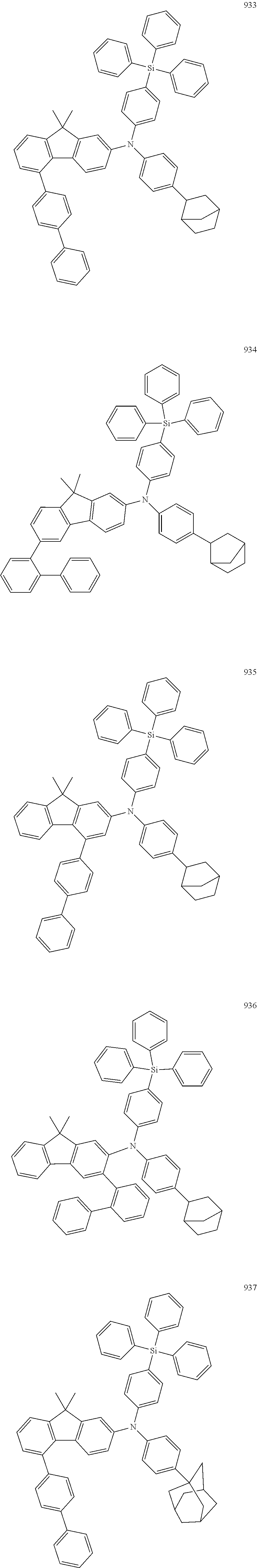

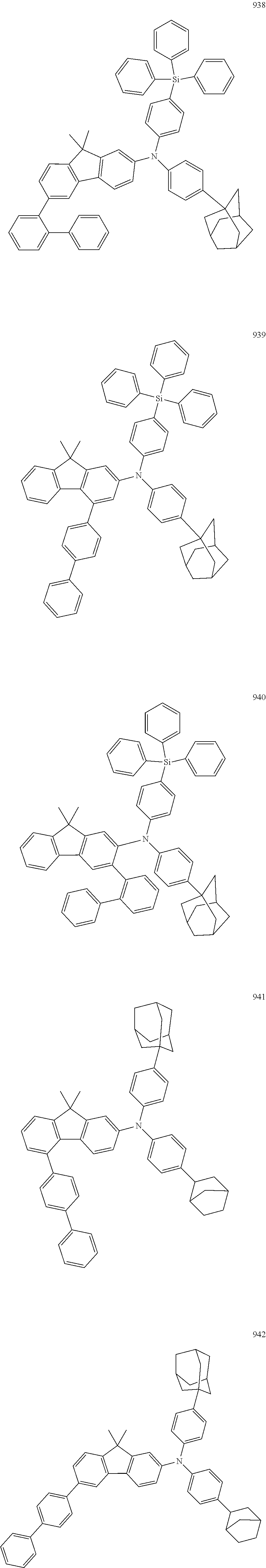

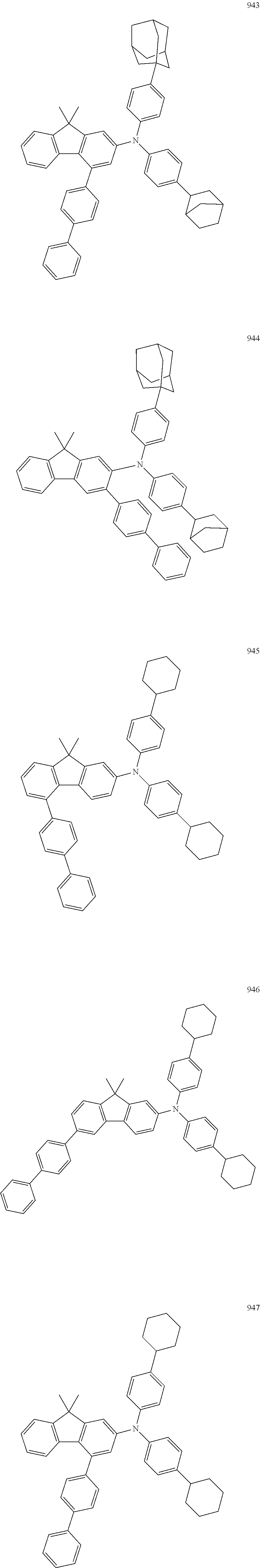 [SMILES: CC1(C)c2ccccc2-c2c(-c3ccc(-c4ccccc4)cc3)cc(N(c3ccc(C4CC5CCC4C5)cc3)c3ccc(C45CC6CC(CC(C6)C4)C5)cc3)cc21 ; CC1(C)c2ccccc2-c2cc(-c3ccc(-c4ccccc4)cc3)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C4CCCCC4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4CCCCC4)cc3)cc21 ; CC1(C)c2ccccc2-c2c(-c3ccc(-c4ccccc4)cc3)cc(N(c3ccc(C4CCCCC4)cc3)c3ccc(C4CCCCC4)cc3)cc21]

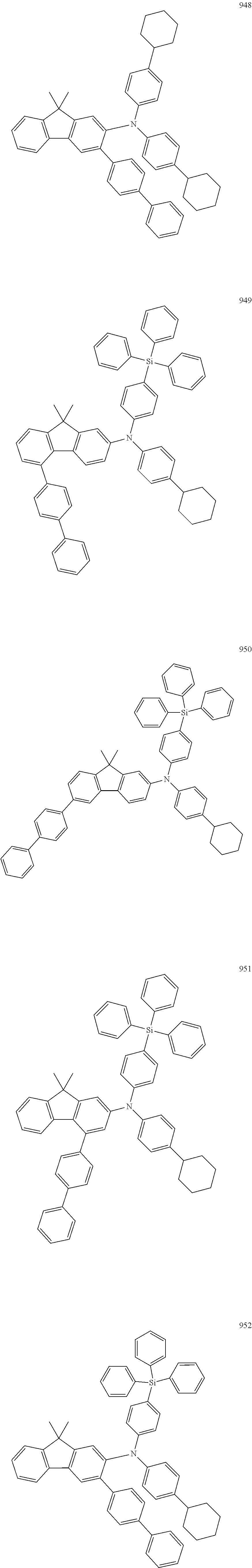

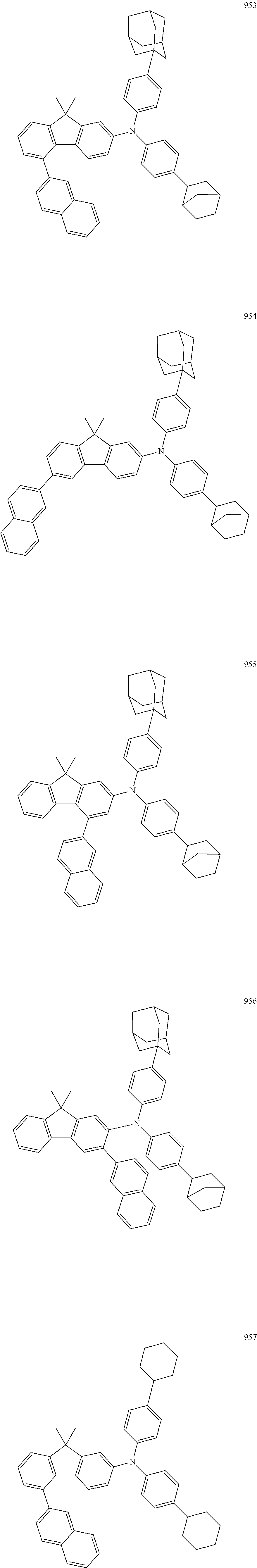 [SMILES: CC1(C)c2cc(N(c3ccc(C4CC5CCC4C5)cc3)c3ccc(C45CC6CC(CC(C6)C4)C5)cc3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3ccc4ccccc4c3)cc(N(c3ccc(C4CC5CCC4C5)cc3)c3ccc(C45CC6CC(CC(C6)C4)C5)cc3)cc21 ; CC1(C)c2ccccc2-c2cc(-c3ccc4ccccc4c3)c(N(c3ccc(C4CC5CCC4C5)cc3)c3ccc(C45CC6CC(CC(C6)C4)C5)cc3)cc21 ; CC1(C)c2cc(N(c3ccc(C4CCCCC4)cc3)c3ccc(C4CCCCC4)cc3)ccc2-c2c(-c3ccc4ccccc4c3)cccc21]

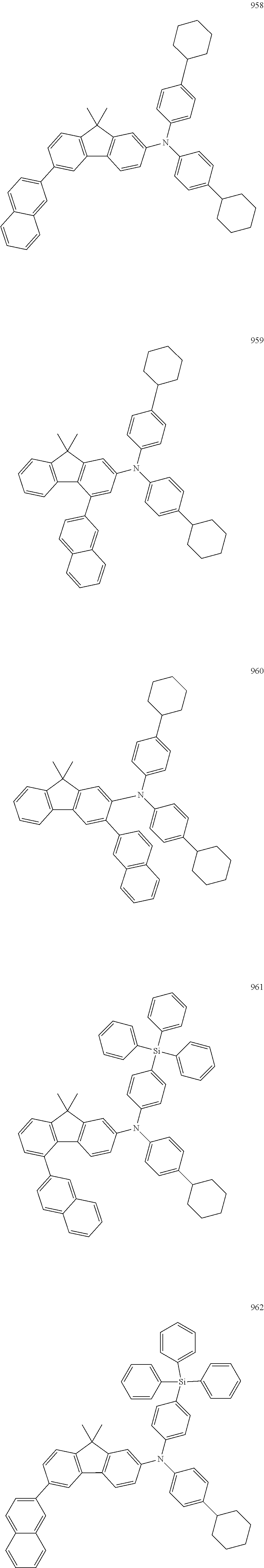 [SMILES: CC1(C)c2ccc(-c3ccc4ccccc4c3)cc2-c2ccc(N(c3ccc(C4CCCCC4)cc3)c3ccc(C4CCCCC4)cc3)cc21 ; CC1(C)c2ccccc2-c2c(-c3ccc4ccccc4c3)cc(N(c3ccc(C4CCCCC4)cc3)c3ccc(C4CCCCC4)cc3)cc21 ; CC1(C)c2ccccc2-c2cc(-c3ccc4ccccc4c3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc([Si](c4ccccc4)(c4ccccc4)c4ccccc4)cc3)cc21]

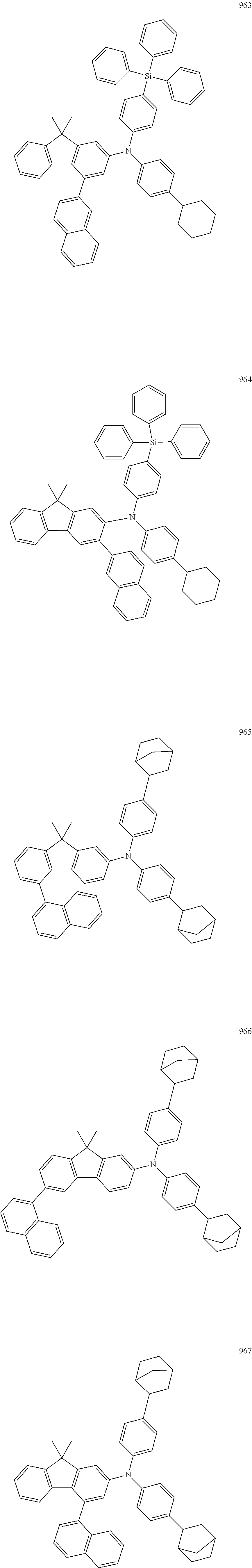 [SMILES: CC1(C)c2ccccc2-c2c(-c3ccc4ccccc4c3)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3ccc4ccccc4c3)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccc(C4CC5CCC4C5)cc3)cc21 ; CC1(C)c2ccccc2-c2c(-c3cccc4ccccc34)cc(N(c3ccc(C4CC5CCC4C5)cc3)c3ccc(C4CC5CCC4C5)cc3)cc21]

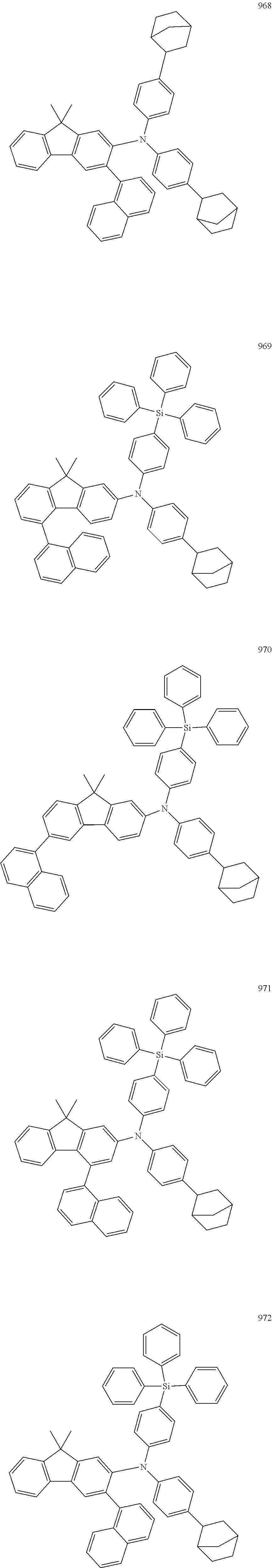

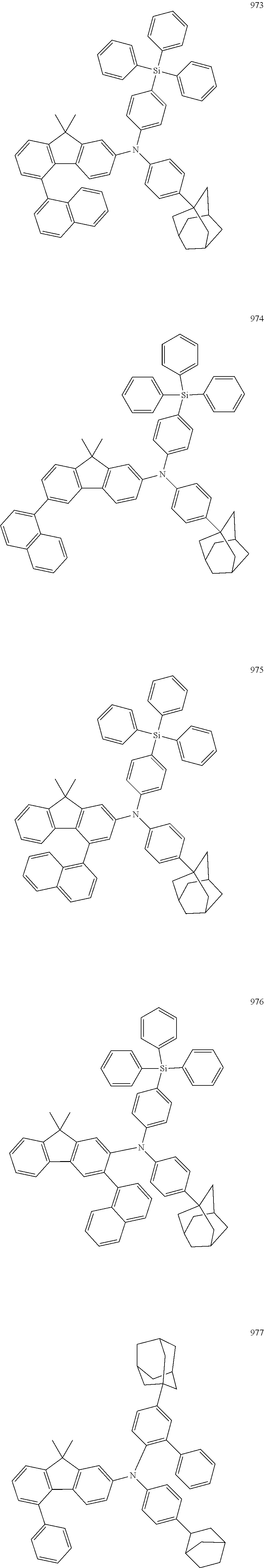

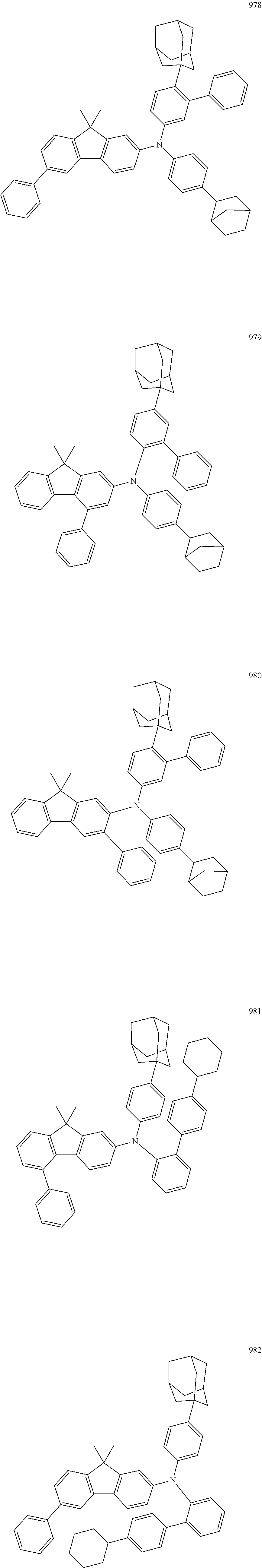

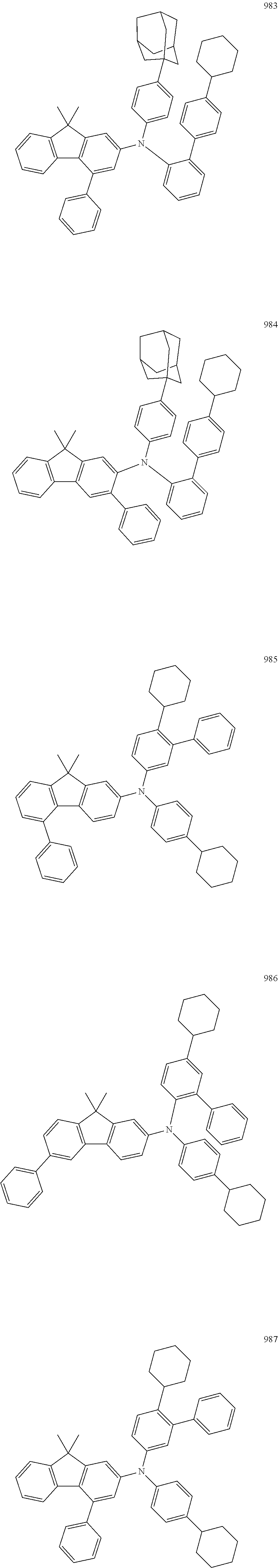 [SMILES: CC1(C)c2ccccc2-c2c(-c3ccccc3)cc(N(c3ccc(C45CC6CC(CC(C6)C4)C5)cc3)c3ccccc3-c3ccc(C4CCCCC4)cc3)cc21 ; CC1(C)c2ccccc2-c2cc(-c3ccccc3)c(N(c3ccc(C45CC6CC(CC(C6)C4)C5)cc3)c3ccccc3-c3ccc(C4CCCCC4)cc3)cc21 ; CC1(C)c2cc(N(c3ccc(C4CCCCC4)cc3)c3ccc(C4CCCCC4)c(-c4ccccc4)c3)ccc2-c2c(-c3ccccc3)cccc21 ; CC1(C)c2ccc(-c3ccccc3)cc2-c2ccc(N(c3ccc(C4CCCCC4)cc3)c3ccc(C4CCCCC4)cc3-c3ccccc3)cc21 ; CC1(C)c2ccccc2-c2c(-c3ccccc3)cc(N(c3ccc(C4CCCCC4)cc3)c3ccc(C4CCCCC4)c(-c4ccccc4)c3)cc21]

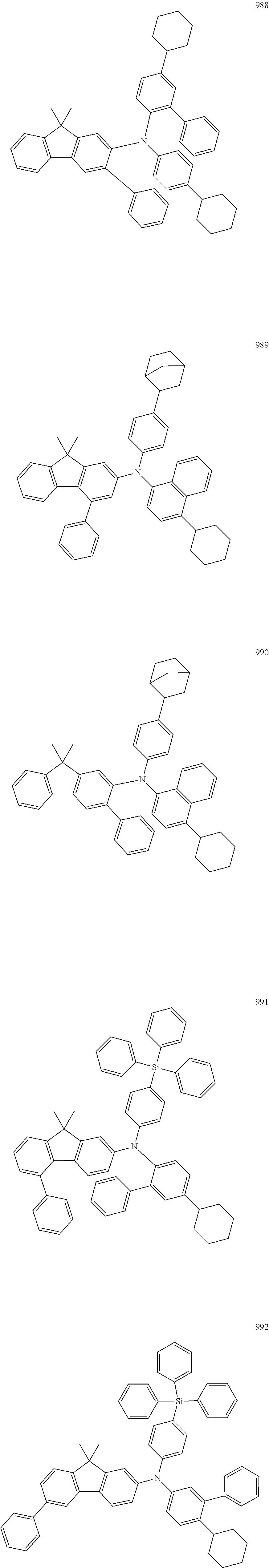 [SMILES: CC1(C)c2ccccc2-c2cc(-c3ccccc3)c(N(c3ccc(C4CCCCC4)cc3)c3ccc(C4CCCCC4)cc3-c3ccccc3)cc21 ; CC1(C)c2ccccc2-c2c(-c3ccccc3)cc(N(c3ccc(C4CC5CCC4C5)cc3)c3ccc(C4CCCCC4)c4ccccc34)cc21 ; CC1(C)c2ccccc2-c2cc(-c3ccccc3)c(N(c3ccc(C4CC5CCC4C5)cc3)c3ccc(C4CCCCC4)c4ccccc34)cc21 ; CC1(C)c2cc(N(c3ccc([Si](c4ccccc4)(c4ccccc4)c4ccccc4)cc3)c3ccc(C4CCCCC4)cc3-c3ccccc3)ccc2-c2c(-c3ccccc3)cccc21 ; CC1(C)c2ccc(-c3ccccc3)cc2-c2ccc(N(c3ccc([Si](c4ccccc4)(c4ccccc4)c4ccccc4)cc3)c3ccc(C4CCCCC4)c(-c4ccccc4)c3)cc21]

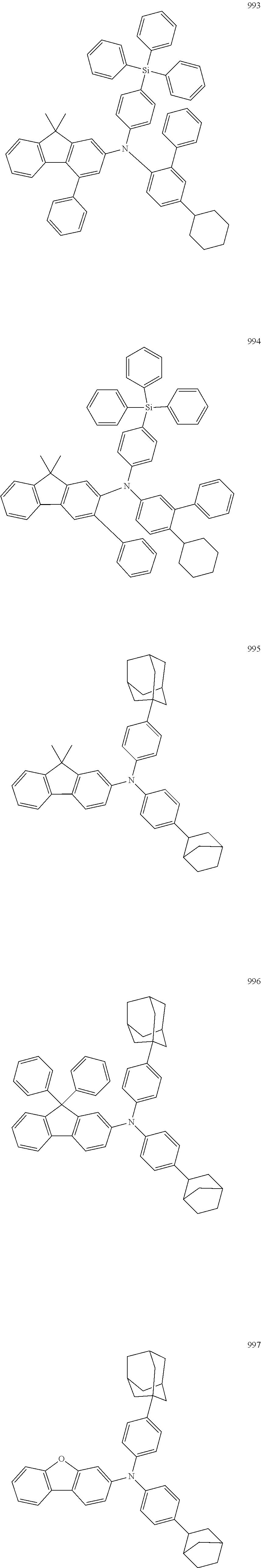 [SMILES: CC1(C)c2ccccc2-c2c(-c3ccccc3)cc(N(c3ccc([Si](c4ccccc4)(c4ccccc4)c4ccccc4)cc3)c3ccc(C4CCCCC4)cc3-c3ccccc3)cc21 ; CC1(C)c2ccccc2-c2cc(-c3ccccc3)c(N(c3ccc([Si](c4ccccc4)(c4ccccc4)c4ccccc4)cc3)c3ccc(C4CCCCC4)c(-c4ccccc4)c3)cc21 ; CC1(C)c2ccccc2-c2ccc(N(c3ccc(C4CC5CCC4C5)cc3)c3ccc(C45CC6CC(CC(C6)C4)C5)cc3)cc21 ; c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1 ; c1ccc2c(c1)oc1cc(N(c3ccc(C4CC5CCC4C5)cc3)c3ccc(C45CC6CC(CC(C6)C4)C5)cc3)ccc12]

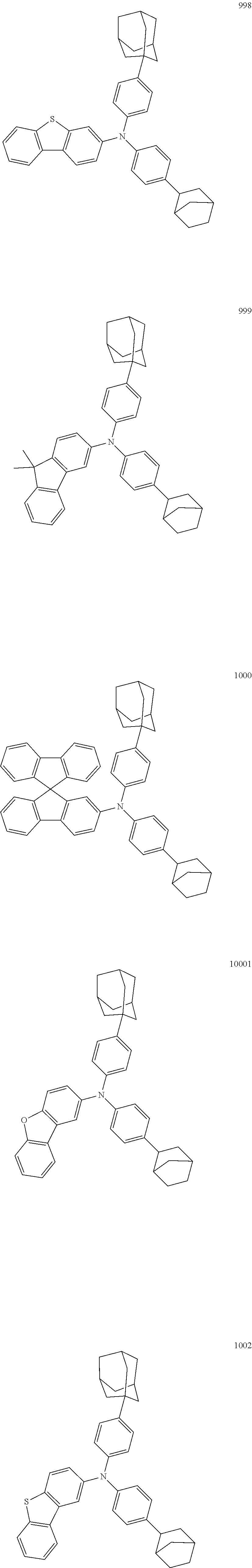 [SMILES: c1ccc2c(c1)sc1cc(N(c3ccc(C4CC5CCC4C5)cc3)c3ccc(C45CC6CC(CC(C6)C4)C5)cc3)ccc12 ; CC1(C)c2ccccc2-c2cc(N(c3ccc(C4CC5CCC4C5)cc3)c3ccc(C45CC6CC(CC(C6)C4)C5)cc3)ccc21 ; c1ccc2c(c1)-c1ccccc1C21c2ccccc2-c2ccc(N(c3ccc(C4CC5CCC4C5)cc3)c3ccc(C45CC6CC(CC(C6)C4)C5)cc3)cc21 ; c1ccc2c(c1)oc1ccc(N(c3ccc(C4CC5CCC4C5)cc3)c3ccc(C45CC6CC(CC(C6)C4)C5)cc3)cc12 ; c1ccc2c(c1)sc1ccc(N(c3ccc(C4CC5CCC4C5)cc3)c3ccc(C45CC6CC(CC(C6)C4)C5)cc3)cc12]

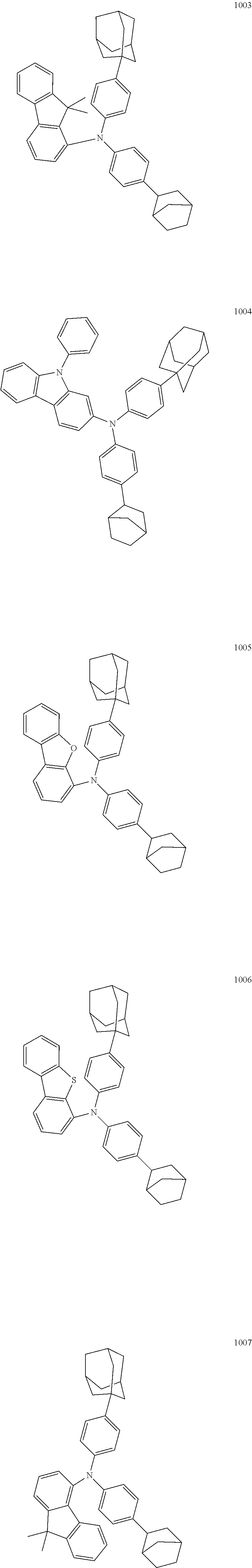 [SMILES: CC1(C)c2ccccc2-c2cccc(N(c3ccc(C4CC5CCC4C5)cc3)c3ccc(C45CC6CC(CC(C6)C4)C5)cc3)c21 ; c1ccc(-n2c3ccccc3c3ccc(N(c4ccc(C5CC6CCC5C6)cc4)c4ccc(C56CC7CC(CC(C7)C5)C6)cc4)cc32)cc1 ; c1ccc2c(c1)oc1c(N(c3ccc(C4CC5CCC4C5)cc3)c3ccc(C45CC6CC(CC(C6)C4)C5)cc3)cccc12 ; c1ccc2c(c1)sc1c(N(c3ccc(C4CC5CCC4C5)cc3)c3ccc(C45CC6CC(CC(C6)C4)C5)cc3)cccc12 ; CC1(C)c2ccccc2-c2c(N(c3ccc(C4CC5CCC4C5)cc3)c3ccc(C45CC6CC(CC(C6)C4)C5)cc3)cccc21]

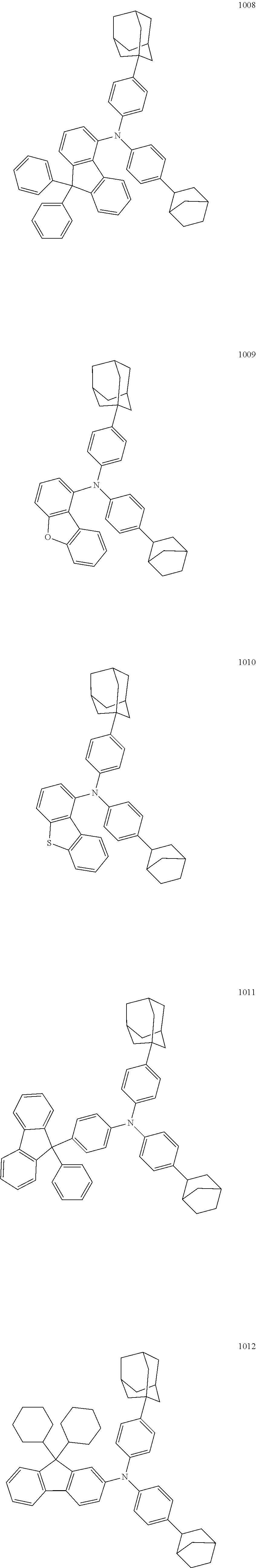 [SMILES: c1ccc(C2(c3ccccc3)c3ccccc3-c3c(N(c4ccc(C5CC6CCC5C6)cc4)c4ccc(C56CC7CC(CC(C7)C5)C6)cc4)cccc32)cc1 ; c1ccc2c(c1)oc1cccc(N(c3ccc(C4CC5CCC4C5)cc3)c3ccc(C45CC6CC(CC(C6)C4)C5)cc3)c12 ; c1ccc2c(c1)sc1cccc(N(c3ccc(C4CC5CCC4C5)cc3)c3ccc(C45CC6CC(CC(C6)C4)C5)cc3)c12 ; c1ccc(C2(c3ccc(N(c4ccc(C5CC6CCC5C6)cc4)c4ccc(C56CC7CC(CC(C7)C5)C6)cc4)cc3)c3ccccc3-c3ccccc32)cc1 ; c1ccc2c(c1)-c1ccc(N(c3ccc(C4CC5CCC4C5)cc3)c3ccc(C45CC6CC(CC(C6)C4)C5)cc3)cc1C2(C1CCCCC1)C1CCCCC1]

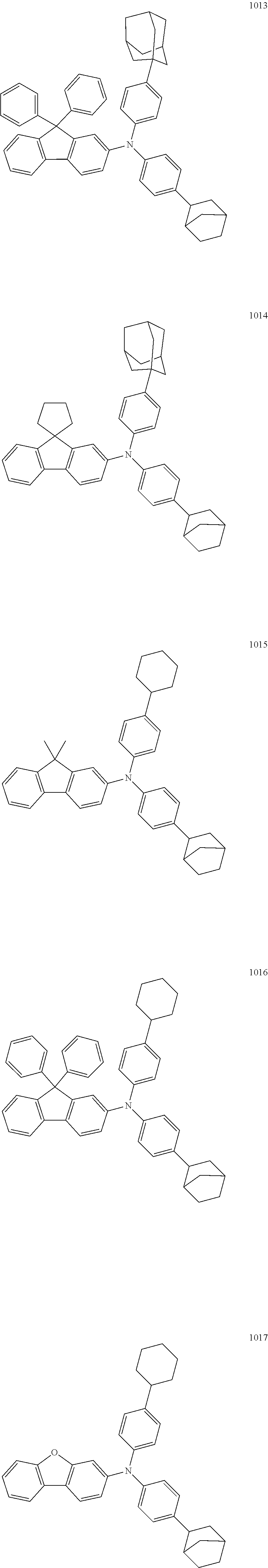 [SMILES: c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1 ; c1ccc2c(c1)-c1ccc(N(c3ccc(C4CC5CCC4C5)cc3)c3ccc(C45CC6CC(CC(C6)C4)C5)cc3)cc1C21CCCC1 ; CC1(C)c2ccccc2-c2ccc(N(c3ccc(C4CCCCC4)cc3)c3ccc(C4CC5CCC4C5)cc3)cc21 ; c1ccc(C2(c3ccccc3)c3ccccc3-c3ccc(N(c4ccc(C5CCCCC5)cc4)c4ccc(C5CC6CCC5C6)cc4)cc32)cc1 ; c1ccc2c(c1)oc1cc(N(c3ccc(C4CCCCC4)cc3)c3ccc(C4CC5CCC4C5)cc3)ccc12]

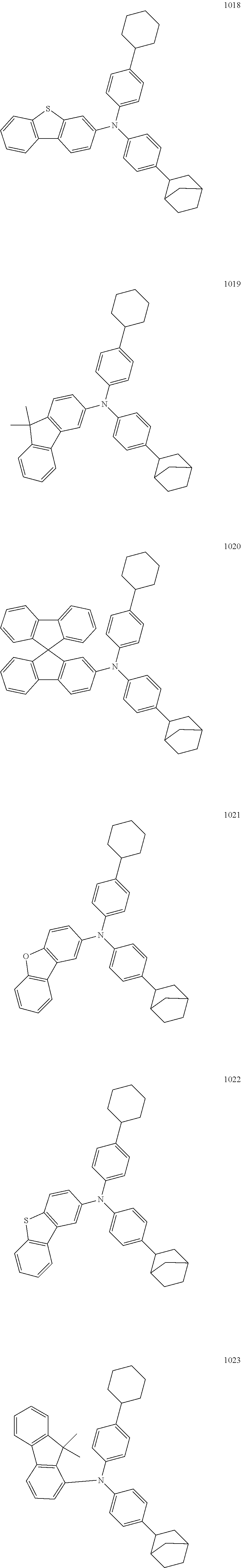 [SMILES: c1ccc2c(c1)sc1cc(N(c3ccc(C4CCCCC4)cc3)c3ccc(C4CC5CCC4C5)cc3)ccc12 ; CC1(C)c2ccccc2-c2cc(N(c3ccc(C4CCCCC4)cc3)c3ccc(C4CC5CCC4C5)cc3)ccc21 ; c1ccc2c(c1)-c1ccccc1C21c2ccccc2-c2ccc(N(c3ccc(C4CCCCC4)cc3)c3ccc(C4CC5CCC4C5)cc3)cc21 ; c1ccc2c(c1)oc1ccc(N(c3ccc(C4CCCCC4)cc3)c3ccc(C4CC5CCC4C5)cc3)cc12 ; c1ccc2c(c1)sc1ccc(N(c3ccc(C4CCCCC4)cc3)c3ccc(C4CC5CCC4C5)cc3)cc12 ; CC1(C)c2ccccc2-c2cccc(N(c3ccc(C4CCCCC4)cc3)c3ccc(C4CC5CCC4C5)cc3)c21]

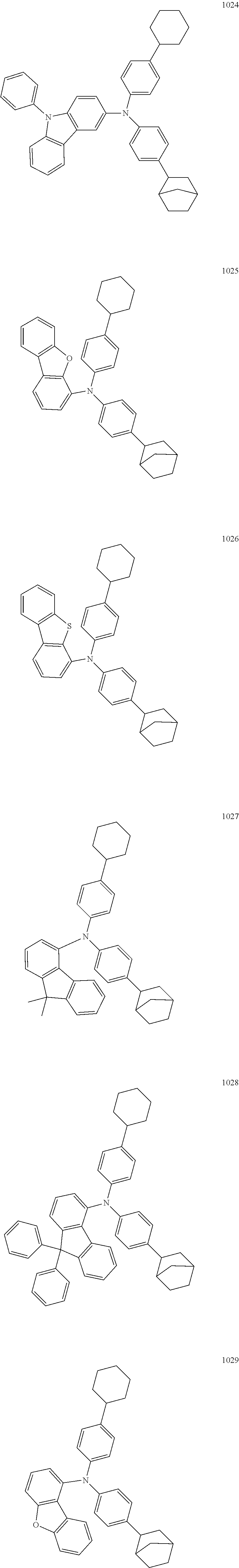 [SMILES: c1ccc(-n2c3ccccc3c3cc(N(c4ccc(C5CCCCC5)cc4)c4ccc(C5CC6CCC5C6)cc4)ccc32)cc1 ; c1ccc2c(c1)oc1c(N(c3ccc(C4CCCCC4)cc3)c3ccc(C4CC5CCC4C5)cc3)cccc12 ; c1ccc2c(c1)sc1c(N(c3ccc(C4CCCCC4)cc3)c3ccc(C4CC5CCC4C5)cc3)cccc12 ; CC1(C)c2ccccc2-c2c(N(c3ccc(C4CCCCC4)cc3)c3ccc(C4CC5CCC4C5)cc3)cccc21 ; c1ccc(C2(c3ccccc3)c3ccccc3-c3c(N(c4ccc(C5CCCCC5)cc4)c4ccc(C5CC6CCC5C6)cc4)cccc32)cc1 ; c1ccc2c(c1)oc1cccc(N(c3ccc(C4CCCCC4)cc3)c3ccc(C4CC5CCC4C5)cc3)c12]

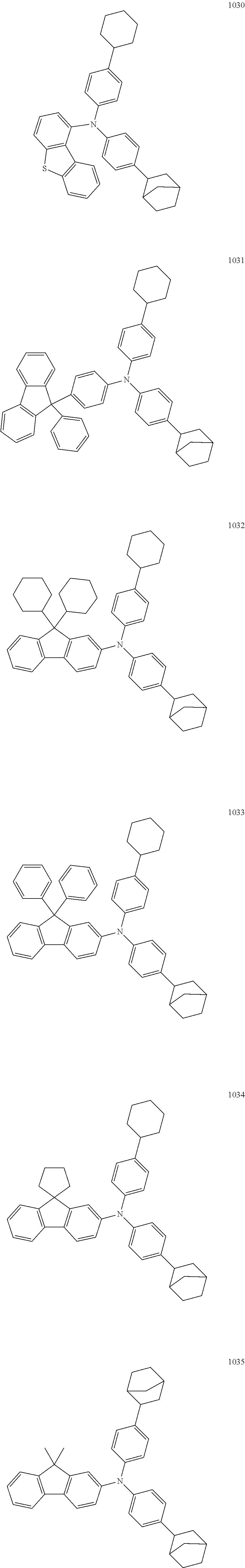 [SMILES: c1ccc2c(c1)sc1cccc(N(c3ccc(C4CCCCC4)cc3)c3ccc(C4CC5CCC4C5)cc3)c12 ; c1ccc(C2(c3ccc(N(c4ccc(C5CCCCC5)cc4)c4ccc(C5CC6CCC5C6)cc4)cc3)c3ccccc3-c3ccccc32)cc1 ; c1ccc2c(c1)-c1ccc(N(c3ccc(C4CCCCC4)cc3)c3ccc(C4CC5CCC4C5)cc3)cc1C2(C1CCCCC1)C1CCCCC1 ; c1ccc(C2(c3ccccc3)c3ccccc3-c3ccc(N(c4ccc(C5CCCCC5)cc4)c4ccc(C5CC6CCC5C6)cc4)cc32)cc1 ; c1ccc2c(c1)-c1ccc(N(c3ccc(C4CCCCC4)cc3)c3ccc(C4CC5CCC4C5)cc3)cc1C21CCCC1 ; CC1(C)c2ccccc2-c2ccc(N(c3ccc(C4CC5CCC4C5)cc3)c3ccc(C4CC5CCC4C5)cc3)cc21]

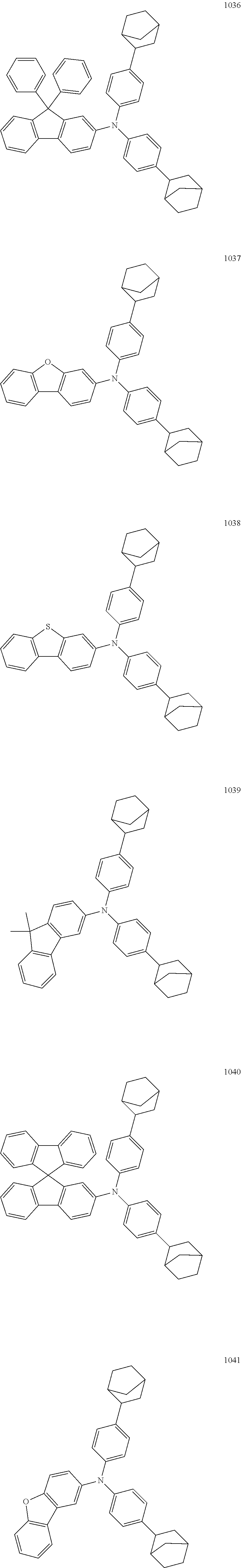 [SMILES: c1ccc(C2(c3ccccc3)c3ccccc3-c3ccc(N(c4ccc(C5CC6CCC5C6)cc4)c4ccc(C5CC6CCC5C6)cc4)cc32)cc1 ; c1ccc2c(c1)oc1cc(N(c3ccc(C4CC5CCC4C5)cc3)c3ccc(C4CC5CCC4C5)cc3)ccc12 ; c1ccc2c(c1)sc1cc(N(c3ccc(C4CC5CCC4C5)cc3)c3ccc(C4CC5CCC4C5)cc3)ccc12 ; CC1(C)c2ccccc2-c2cc(N(c3ccc(C4CC5CCC4C5)cc3)c3ccc(C4CC5CCC4C5)cc3)ccc21 ; c1ccc2c(c1)-c1ccccc1C21c2ccccc2-c2ccc(N(c3ccc(C4CC5CCC4C5)cc3)c3ccc(C4CC5CCC4C5)cc3)cc21 ; c1ccc2c(c1)oc1ccc(N(c3ccc(C4CC5CCC4C5)cc3)c3ccc(C4CC5CCC4C5)cc3)cc12]

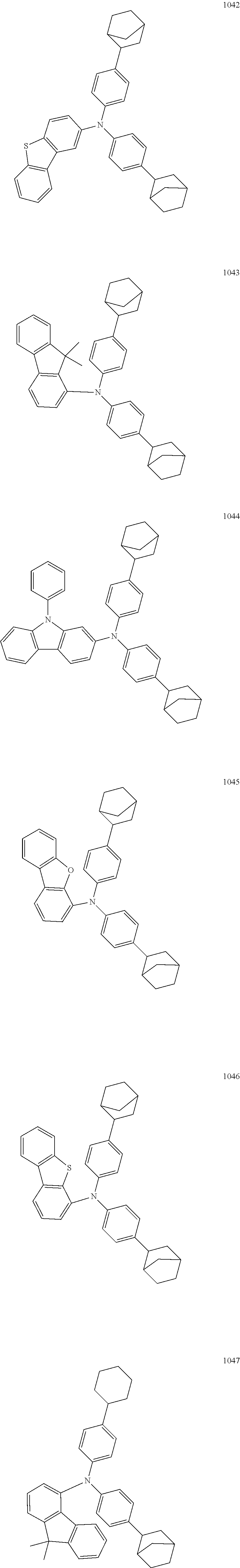 [SMILES: c1ccc2c(c1)sc1ccc(N(c3ccc(C4CC5CCC4C5)cc3)c3ccc(C4CC5CCC4C5)cc3)cc12 ; CC1(C)c2ccccc2-c2cccc(N(c3ccc(C4CC5CCC4C5)cc3)c3ccc(C4CC5CCC4C5)cc3)c21 ; c1ccc(-n2c3ccccc3c3ccc(N(c4ccc(C5CC6CCC5C6)cc4)c4ccc(C5CC6CCC5C6)cc4)cc32)cc1 ; c1ccc2c(c1)oc1c(N(c3ccc(C4CC5CCC4C5)cc3)c3ccc(C4CC5CCC4C5)cc3)cccc12 ; c1ccc2c(c1)sc1c(N(c3ccc(C4CC5CCC4C5)cc3)c3ccc(C4CC5CCC4C5)cc3)cccc12 ; CC1(C)c2ccccc2-c2c(N(c3ccc(C4CCCCC4)cc3)c3ccc(C4CC5CCC4C5)cc3)cccc21]

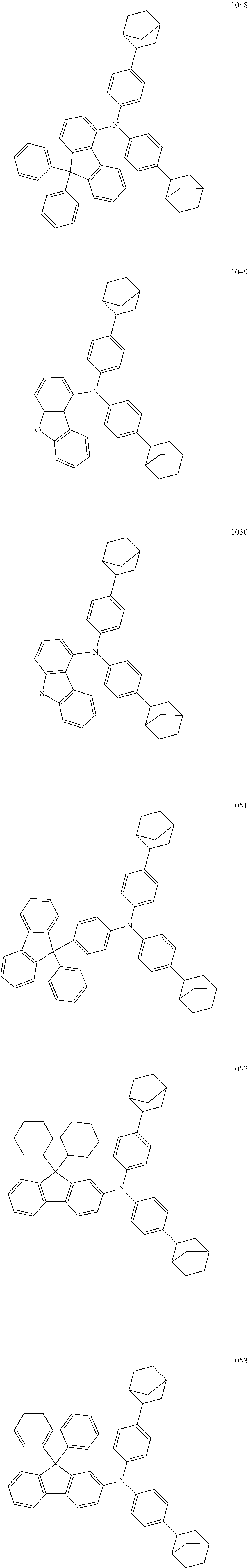 [SMILES: c1ccc(C2(c3ccccc3)c3ccccc3-c3c(N(c4ccc(C5CC6CCC5C6)cc4)c4ccc(C5CC6CCC5C6)cc4)cccc32)cc1 ; c1ccc2c(c1)oc1cccc(N(c3ccc(C4CC5CCC4C5)cc3)c3ccc(C4CC5CCC4C5)cc3)c12 ; c1ccc2c(c1)sc1cccc(N(c3ccc(C4CC5CCC4C5)cc3)c3ccc(C4CC5CCC4C5)cc3)c12 ; c1ccc(C2(c3ccc(N(c4ccc(C5CC6CCC5C6)cc4)c4ccc(C5CC6CCC5C6)cc4)cc3)c3ccccc3-c3ccccc32)cc1 ; c1ccc2c(c1)-c1ccc(N(c3ccc(C4CC5CCC4C5)cc3)c3ccc(C4CC5CCC4C5)cc3)cc1C2(C1CCCCC1)C1CCCCC1 ; c1ccc(C2(c3ccccc3)c3ccccc3-c3ccc(N(c4ccc(C5CC6CCC5C6)cc4)c4ccc(C5CC6CCC5C6)cc4)cc32)cc1]

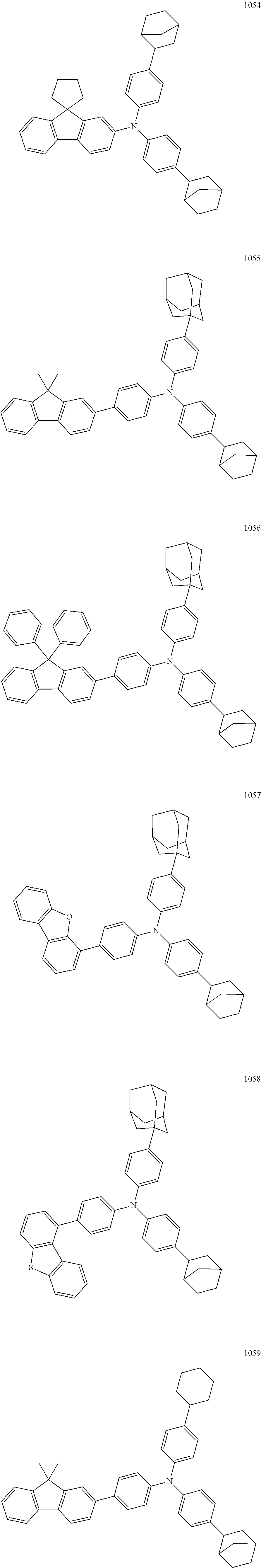

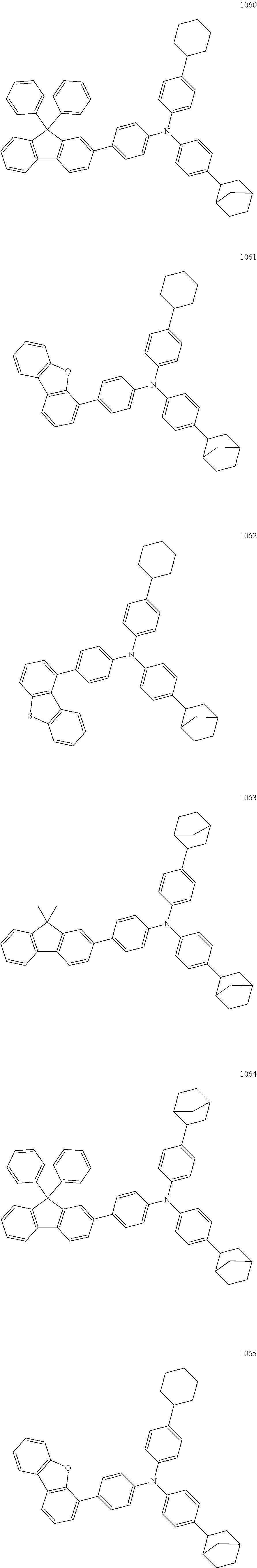

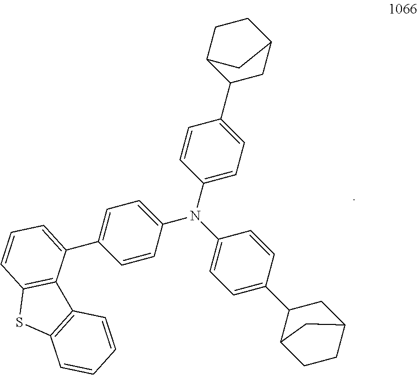 [SMILES: c1ccc2c(c1)sc1cccc(-c3ccc(N(c4ccc(C5CC6CCC5C6)cc4)c4ccc(C5CC6CCC5C6)cc4)cc3)c12]

13. The light emitting device of claim 1, wherein the at least one functional layer comprises an emission layer, a first hole transport layer between the first electrode and the emission layer, a second hole transport layer between the first hole transport layer and the emission layer, and an electron transport region between the emission layer and the second electrode, and
the first hole transport layer comprises the amine compound.

14. The light emitting device of claim 13, wherein the at least one functional layer further comprises a third hole transport layer between the second hole transport layer and the emission layer, and
the third hole transport layer comprises the amine compound.

15. The light emitting device of claim 13, wherein the second hole transport layer comprises an amine derivative compound represented by the following Formula 10: 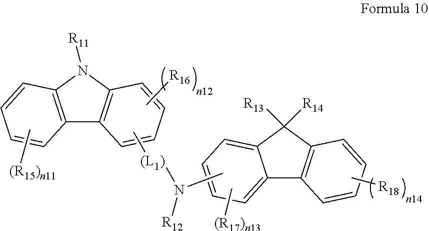
in Formula 10,
L₁ is a direct linkage, a substituted or unsubstituted arylene group of 6 to 30 ring-forming carbon atoms, or a substituted or unsubstituted heteroarylene group of 2 to 30 ring-forming carbon atoms,
R₁₁ to R₁₄ are each independently a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
R₁₅ to R₁₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
n11 and n14 are each independently an integer of 0 to 4, and
n12 and n13 are each independently an integer of 0 to 3.

16. The light emitting device of claim 15, wherein the amine derivative compound represented by Formula 10 is represented by the following Formula 11-1 or Formula 11-2: 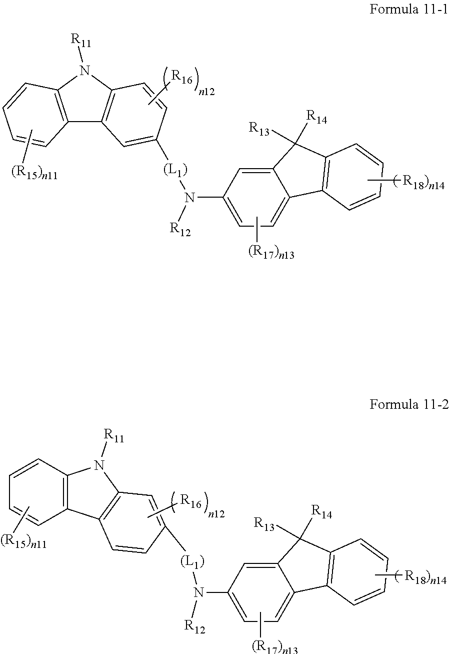
in Formula 11-1 and Formula 11-2, L₁, R₁₁ to R₁₄, R₁₅ to R₁₈, and n11 to n13 are the same as defined with respect to Formula 10.

17. The light emitting device of claim 15, wherein the amine derivative compound represented by Formula 10 is represented by any one selected from among compounds represented in the following Compound Group 2:

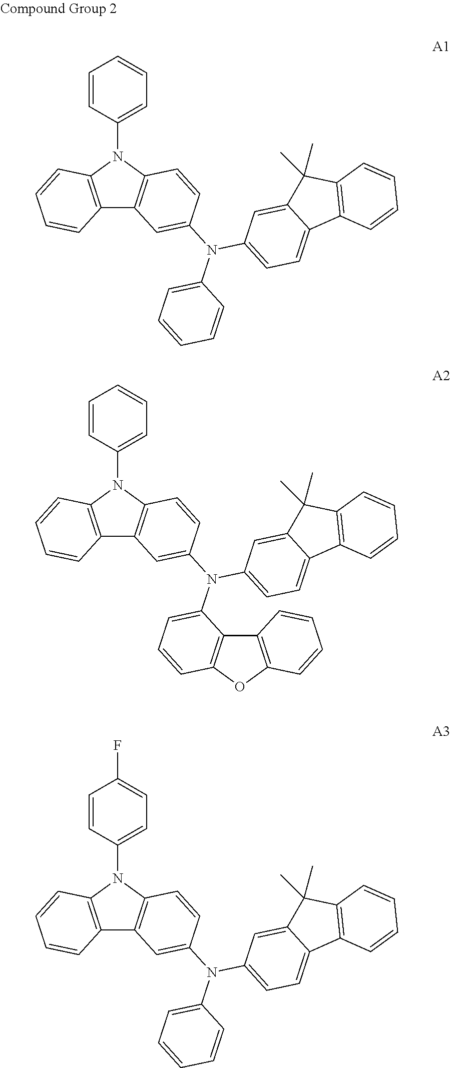 [SMILES: CC1(C)c2ccccc2-c2ccc(N(c3ccccc3)c3ccc4c(c3)c3ccccc3n4-c3ccccc3)cc21 ; CC1(C)c2ccccc2-c2ccc(N(c3ccc4c(c3)c3ccccc3n4-c3ccccc3)c3cccc4oc5ccccc5c34)cc21 ; CC1(C)c2ccccc2-c2ccc(N(c3ccccc3)c3ccc4c(c3)c3ccccc3n4-c3ccc(F)cc3)cc21]

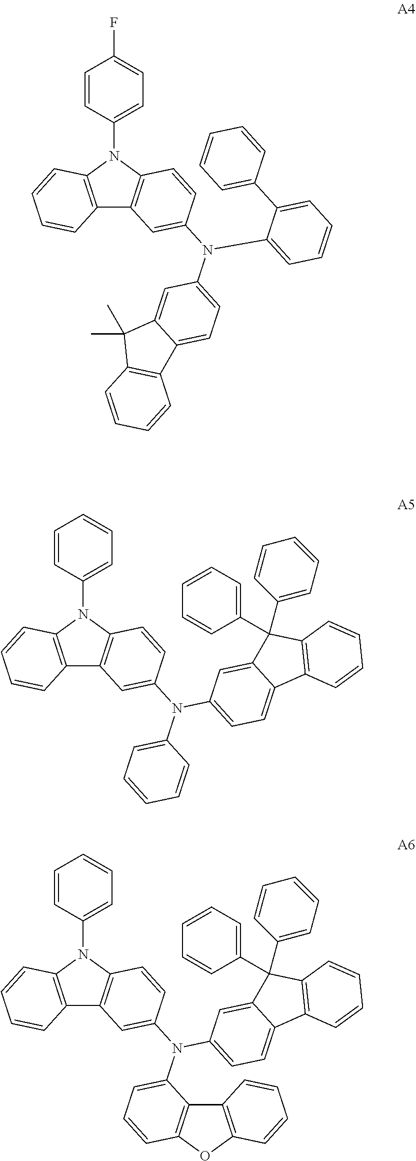 [SMILES: CC1(C)c2ccccc2-c2ccc(N(c3ccc4c(c3)c3ccccc3n4-c3ccc(F)cc3)c3ccccc3-c3ccccc3)cc21 ; c1ccc(N(c2ccc3c(c2)C(c2ccccc2)(c2ccccc2)c2ccccc2-3)c2ccc3c(c2)c2ccccc2n3-c2ccccc2)cc1 ; c1ccc(-n2c3ccccc3c3cc(N(c4ccc5c(c4)C(c4ccccc4)(c4ccccc4)c4ccccc4-5)c4cccc5oc6ccccc6c45)ccc32)cc1]

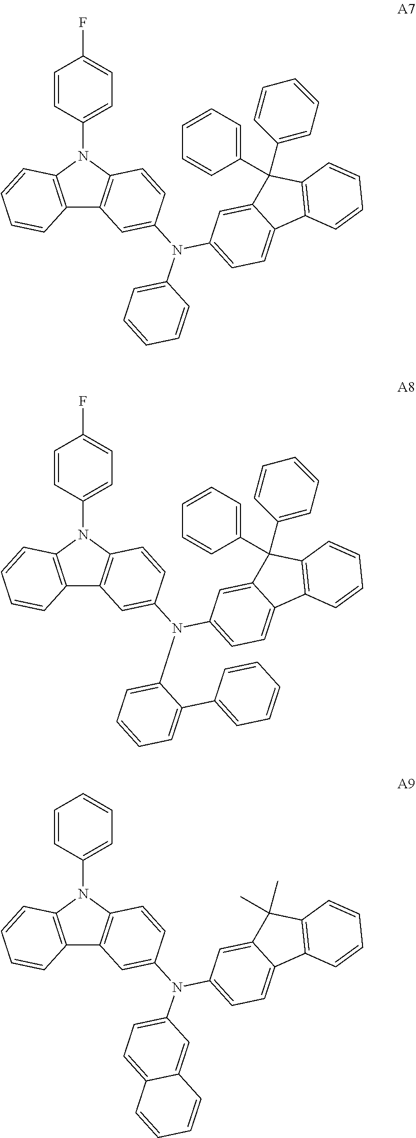 [SMILES: Fc1ccc(-n2c3ccccc3c3cc(N(c4ccccc4)c4ccc5c(c4)C(c4ccccc4)(c4ccccc4)c4ccccc4-5)ccc32)cc1 ; Fc1ccc(-n2c3ccccc3c3cc(N(c4ccc5c(c4)C(c4ccccc4)(c4ccccc4)c4ccccc4-5)c4ccccc4-c4ccccc4)ccc32)cc1 ; CC1(C)c2ccccc2-c2ccc(N(c3ccc4ccccc4c3)c3ccc4c(c3)c3ccccc3n4-c3ccccc3)cc21]

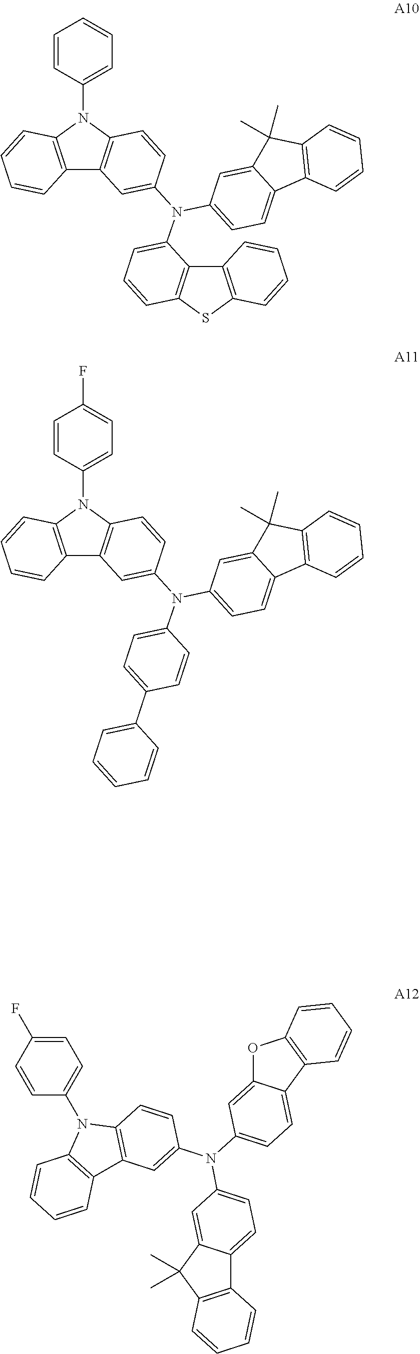 [SMILES: CC1(C)c2ccccc2-c2ccc(N(c3ccc4c(c3)c3ccccc3n4-c3ccccc3)c3cccc4sc5ccccc5c34)cc21 ; CC1(C)c2ccccc2-c2ccc(N(c3ccc(-c4ccccc4)cc3)c3ccc4c(c3)c3ccccc3n4-c3ccc(F)cc3)cc21 ; CC1(C)c2ccccc2-c2ccc(N(c3ccc4c(c3)oc3ccccc34)c3ccc4c(c3)c3ccccc3n4-c3ccc(F)cc3)cc21]

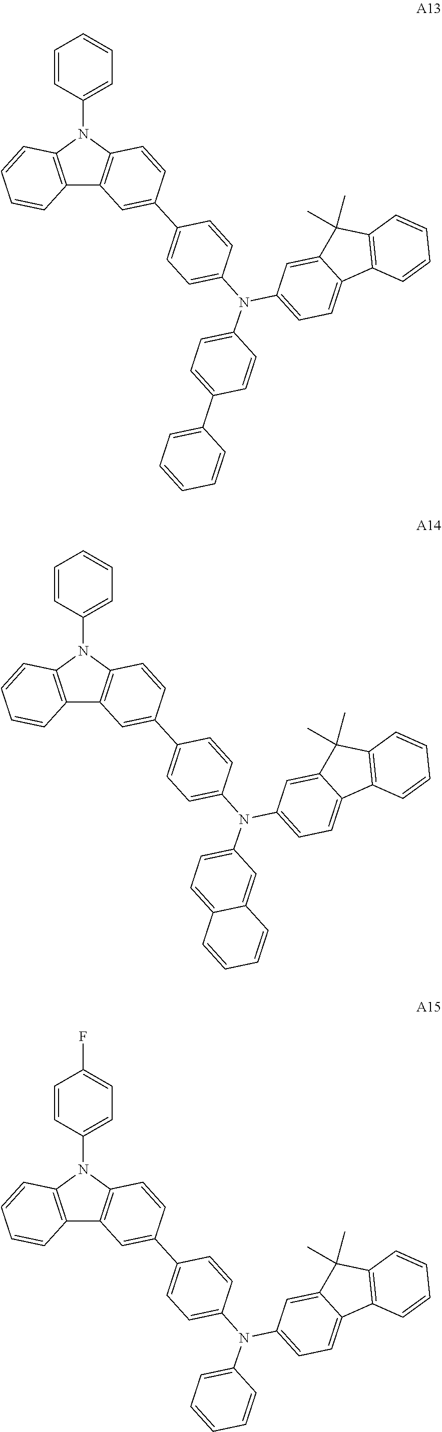 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3ccc4ccccc4c3)cc21 ; CC1(C)c2ccccc2-c2ccc(N(c3ccccc3)c3ccc(-c4ccc5c(c4)c4ccccc4n5-c4ccc(F)cc4)cc3)cc21]

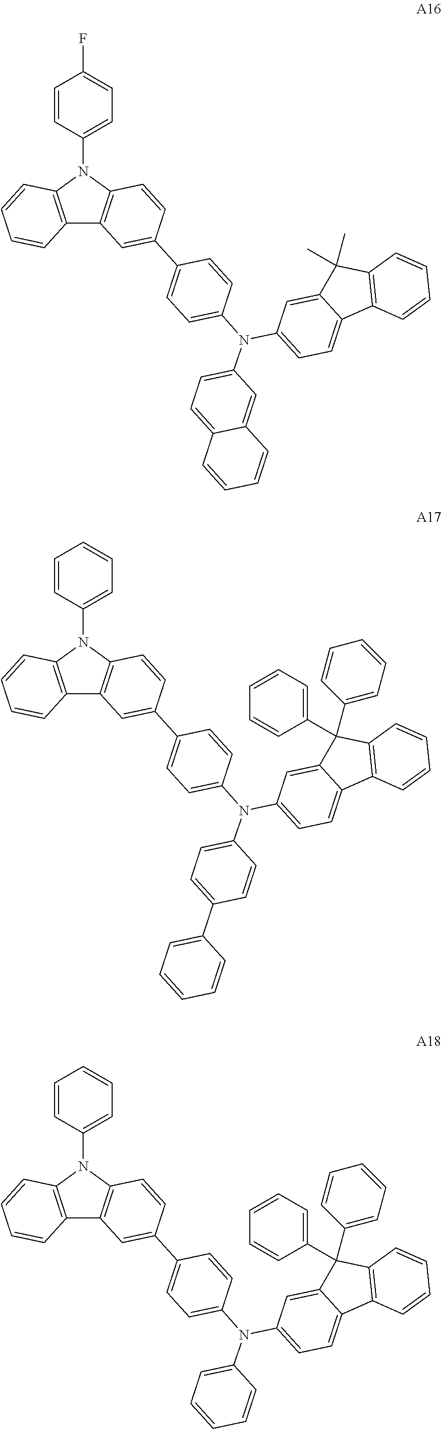 [SMILES: CC1(C)c2ccccc2-c2ccc(N(c3ccc(-c4ccc5c(c4)c4ccccc4n5-c4ccc(F)cc4)cc3)c3ccc4ccccc4c3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

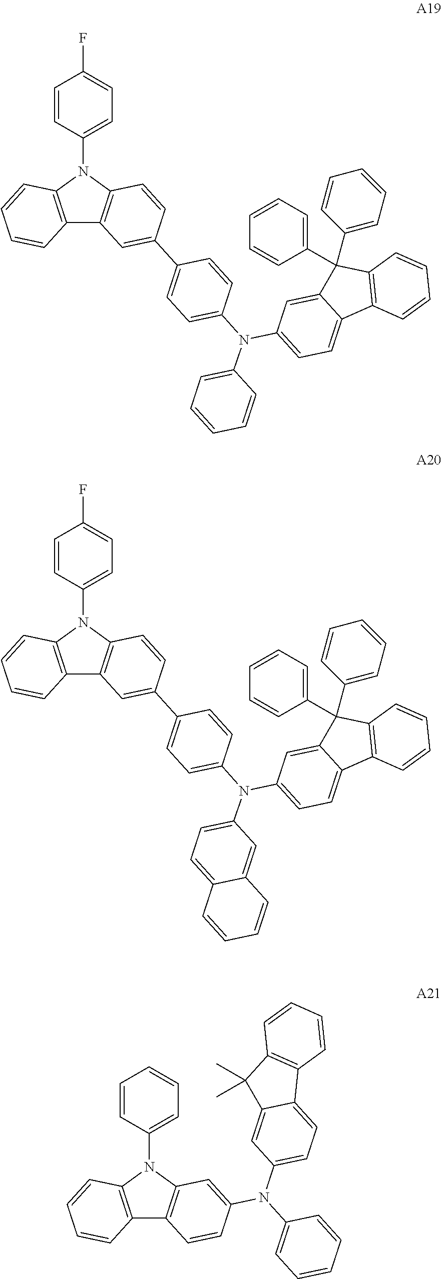 [SMILES: Fc1ccc(-n2c3ccccc3c3cc(-c4ccc(N(c5ccccc5)c5ccc6c(c5)C(c5ccccc5)(c5ccccc5)c5ccccc5-6)cc4)ccc32)cc1 ; Fc1ccc(-n2c3ccccc3c3cc(-c4ccc(N(c5ccc6c(c5)C(c5ccccc5)(c5ccccc5)c5ccccc5-6)c5ccc6ccccc6c5)cc4)ccc32)cc1 ; CC1(C)c2ccccc2-c2ccc(N(c3ccccc3)c3ccc4c5ccccc5n(-c5ccccc5)c4c3)cc21]

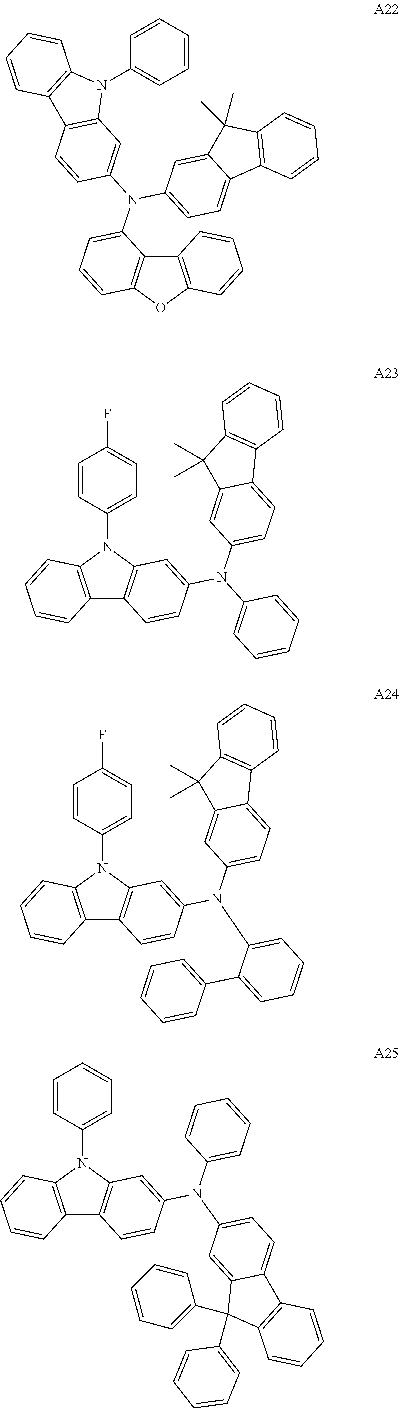 [SMILES: CC1(C)c2ccccc2-c2ccc(N(c3ccc4c5ccccc5n(-c5ccccc5)c4c3)c3cccc4oc5ccccc5c34)cc21 ; CC1(C)c2ccccc2-c2ccc(N(c3ccccc3)c3ccc4c5ccccc5n(-c5ccc(F)cc5)c4c3)cc21 ; CC1(C)c2ccccc2-c2ccc(N(c3ccc4c5ccccc5n(-c5ccc(F)cc5)c4c3)c3ccccc3-c3ccccc3)cc21 ; c1ccc(N(c2ccc3c(c2)C(c2ccccc2)(c2ccccc2)c2ccccc2-3)c2ccc3c4ccccc4n(-c4ccccc4)c3c2)cc1]

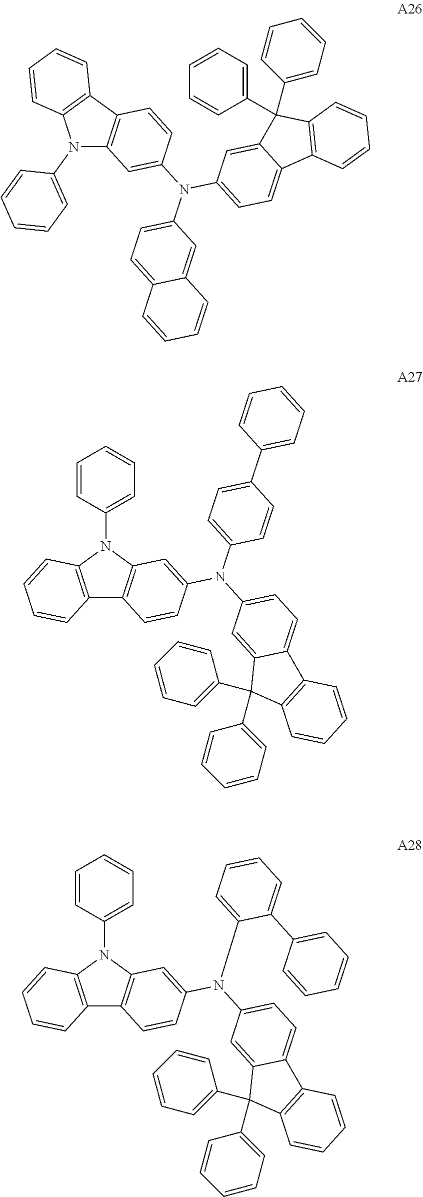 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; c1ccc(-c2ccccc2N(c2ccc3c(c2)C(c2ccccc2)(c2ccccc2)c2ccccc2-3)c2ccc3c4ccccc4n(-c4ccccc4)c3c2)cc1]

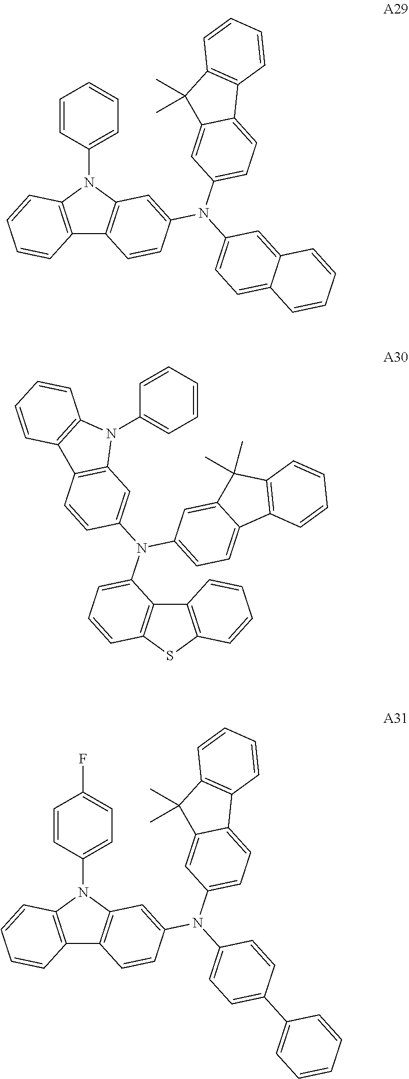 [SMILES: CC1(C)c2ccccc2-c2ccc(N(c3ccc4ccccc4c3)c3ccc4c5ccccc5n(-c5ccccc5)c4c3)cc21 ; CC1(C)c2ccccc2-c2ccc(N(c3ccc4c5ccccc5n(-c5ccccc5)c4c3)c3cccc4sc5ccccc5c34)cc21 ; CC1(C)c2ccccc2-c2ccc(N(c3ccc(-c4ccccc4)cc3)c3ccc4c5ccccc5n(-c5ccc(F)cc5)c4c3)cc21]

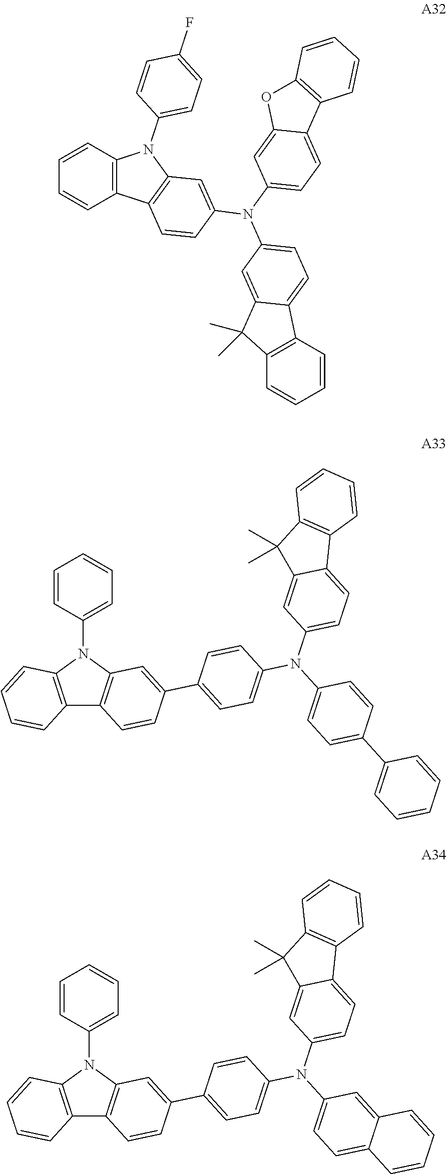 [SMILES: CC1(C)c2ccccc2-c2ccc(N(c3ccc4c(c3)oc3ccccc34)c3ccc4c5ccccc5n(-c5ccc(F)cc5)c4c3)cc21 ; CC1(C)c2ccccc2-c2ccc(N(c3ccc(-c4ccccc4)cc3)c3ccc(-c4ccc5c6ccccc6n(-c6ccccc6)c5c4)cc3)cc21 ; CC1(C)c2ccccc2-c2ccc(N(c3ccc(-c4ccc5c6ccccc6n(-c6ccccc6)c5c4)cc3)c3ccc4ccccc4c3)cc21]

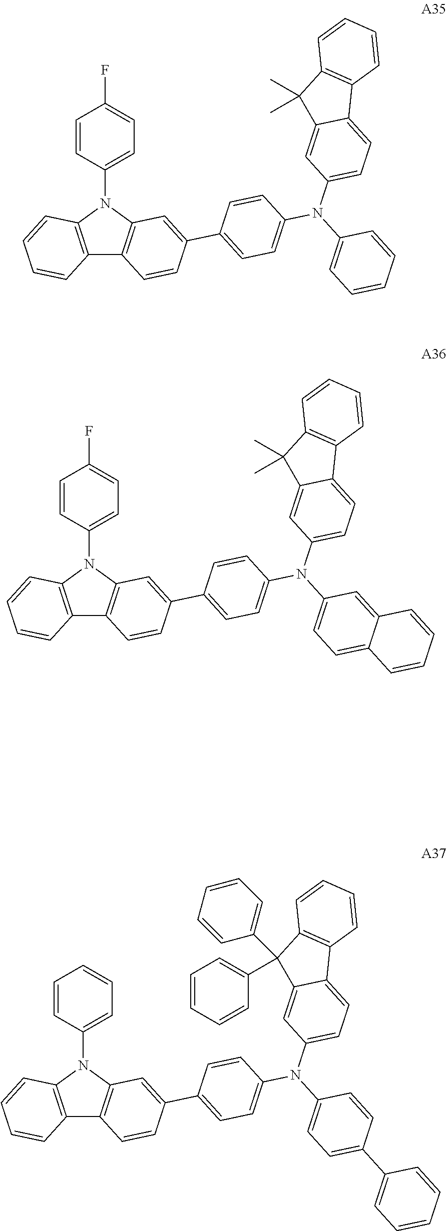 [SMILES: CC1(C)c2ccccc2-c2ccc(N(c3ccccc3)c3ccc(-c4ccc5c6ccccc6n(-c6ccc(F)cc6)c5c4)cc3)cc21 ; CC1(C)c2ccccc2-c2ccc(N(c3ccc(-c4ccc5c6ccccc6n(-c6ccc(F)cc6)c5c4)cc3)c3ccc4ccccc4c3)cc21 ; c1ccc(-c2ccc(N(c3ccc(-c4ccc5c6ccccc6n(-c6ccccc6)c5c4)cc3)c3ccc4c(c3)C(c3ccccc3)(c3ccccc3)c3ccccc3-4)cc2)cc1]

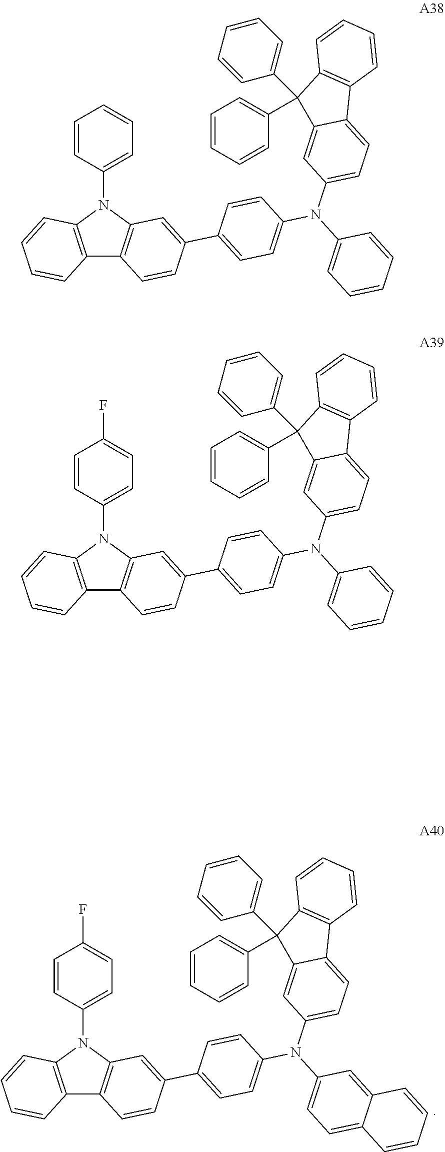 [SMILES: c1ccc(N(c2ccc(-c3ccc4c5ccccc5n(-c5ccccc5)c4c3)cc2)c2ccc3c(c2)C(c2ccccc2)(c2ccccc2)c2ccccc2-3)cc1 ; Fc1ccc(-n2c3ccccc3c3ccc(-c4ccc(N(c5ccccc5)c5ccc6c(c5)C(c5ccccc5)(c5ccccc5)c5ccccc5-6)cc4)cc32)cc1 ; Fc1ccc(-n2c3ccccc3c3ccc(-c4ccc(N(c5ccc6c(c5)C(c5ccccc5)(c5ccccc5)c5ccccc5-6)c5ccc6ccccc6c5)cc4)cc32)cc1]

18. The light emitting device of claim 1, wherein the at least one functional layer comprises an emission layer, a hole transport layer between the first electrode and the emission layer, and a hole transport auxiliary layer between the hole transport layer and the emission layer, and
the hole transport auxiliary layer comprises the amine compound.

19. The light emitting device of claim 18, wherein the hole transport auxiliary layer comprises:
a first hole transport auxiliary layer on the hole transport layer; and
a second hole transport auxiliary layer on the first hole transport auxiliary layer, and
the first hole transport auxiliary layer comprises the amine compound.

20. The light emitting device of claim 19, wherein a refractive index of the first hole transport auxiliary layer in a wavelength range of about 450 nm to about 700 nm is about 1.55 to about 1.80, and
a refractive index of the second hole transport auxiliary layer in a wavelength range of about 450 nm to about 700 nm is about 1.65 to about 1.90.

21. The light emitting device of claim 19, wherein an absolute value of the highest occupied molecular orbital (HOMO) energy level of the second hole transport auxiliary layer is greater than an absolute value of the HOMO energy level of the first hole transport auxiliary layer.

22. An amine compound represented by the following Formula 1: 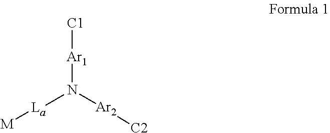
in Formula 1,
Ar₁ and Ar₂ are each independently a substituted or unsubstituted arylene group of 6 to 30 ring-forming carbon atoms, or a substituted or unsubstituted heteroarylene group of 2 to 30 ring-forming carbon atoms,
Lₐ is a direct linkage, a substituted or unsubstituted arylene group of 6 to 30 ring-forming carbon atoms, or a substituted or unsubstituted heteroarylene group of 2 to 30 ring-forming carbon atoms,
C1 and C2 are each independently a substituted or unsubstituted adamantyl group, a substituted or unsubstituted cyclohexyl group, a substituted or unsubstituted bicycloheptanyl group, a substituted or unsubstituted bicyclooctanyl group, or a substituted or unsubstituted triphenylsilyl group,
a case where both C1 and C2 are substituted or unsubstituted adamantyl groups is excluded, and
M is represented by any one selected from among the following Formula 1-a to Formula 1-c: 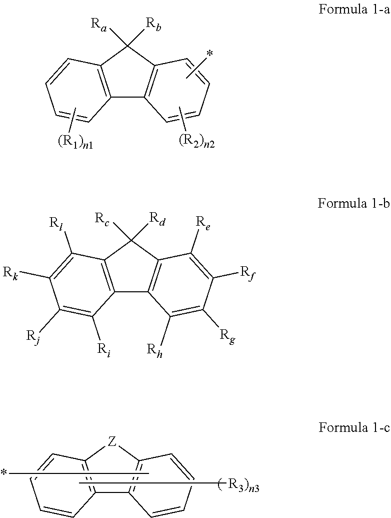
in Formula 1-a to Formula 1-c,
Z is NR₄, O, or S,
R₁ and R₂ are each independently a substituted or unsubstituted aryl group of 6 to 30 ring-forming carbon atoms, or a substituted or unsubstituted heteroaryl group of 2 to 30 ring-forming carbon atoms, and
Rₐ to Rₗ, R₃, and R₄ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
in Formula 1-b, any one selected from among R_{c} to Rₗ is a part connected with Formula 1,
in Formula 1-a and Formula 1-c, 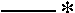 [SMILES: *C]
is a part connected with Formula 1,
in a case where M in Formula 1 is represented by Formula 1-b or Formula 1-c, at least one selected from among C1 and C2 in Formula 1 is a bicycloheptanyl group,
n1 is an integer of 0 to 4,
n2 is an integer of 0 to 3,
where the sum of n1 and n2 is 1 or more, and
n3 is an integer of 0 to 7.

23. The amine compound of claim 22, wherein the amine compound represented by Formula 1 is represented by any one selected from among the following Formula 2-1 to Formula 2-8: 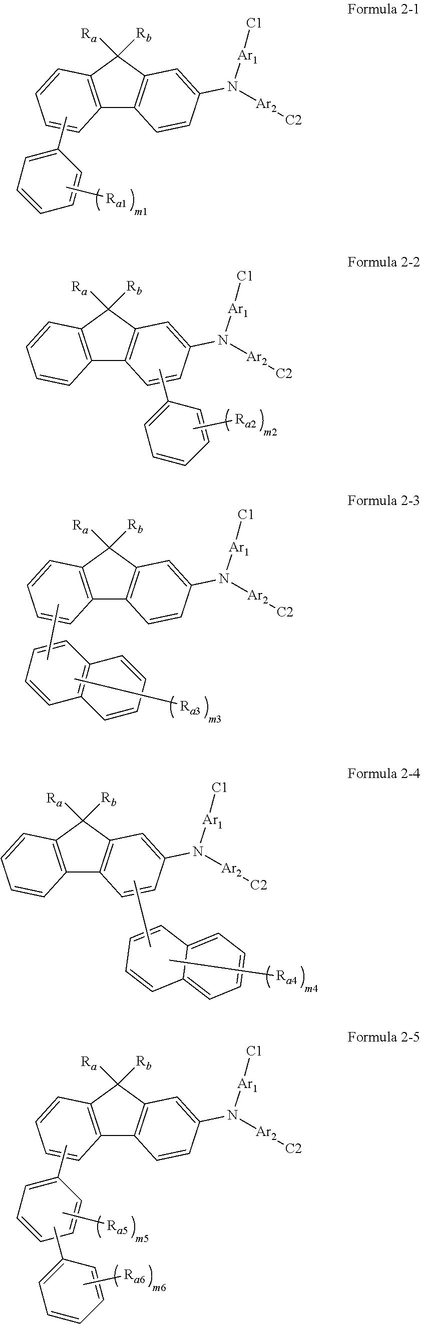 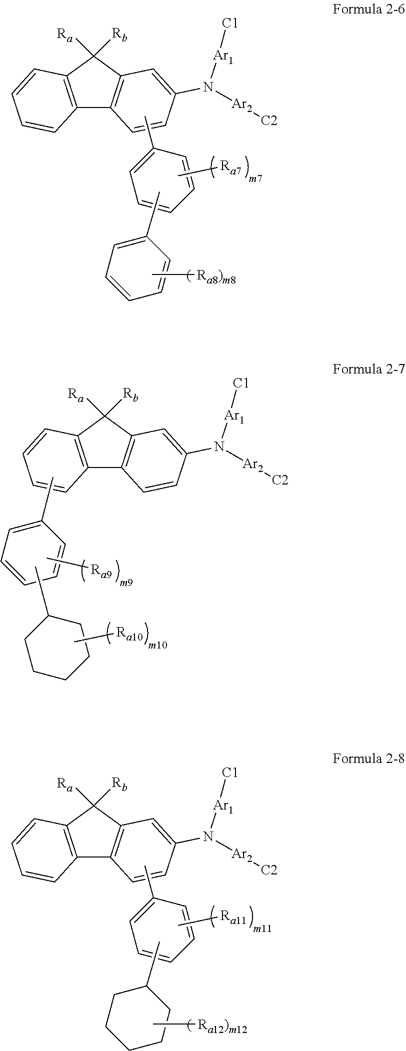
in Formula 2-1 to Formula 2-8,
Rₐ₁ to Rₐ₁₂ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m1, m2, m6, and m8″ are each independently an integer of 0 to 5,
m3 and m4 are each independently an integer of 0 to 7,
m5, m7, m9, and m11 are each independently an integer of 0 to 4,
m10, and m12 are each independently an integer of 0 to 11, and
Rₐ, R_{b}, Ar₁, Ar₂, C1 and C2 are the same as defined with respect to Formula 1 and Formula 1-a.

24. The amine compound of claim 22, wherein the amine compound represented by Formula 1 is represented by the following Formula 3-1 or Formula 3-2: 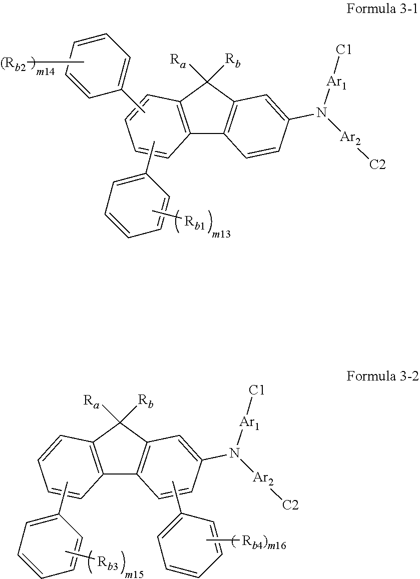
in Formula 3-1 and Formula 3-2,
R_{b1} to R_{b4} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m13 to m16 are each independently an integer of 0 to 5, and
Rₐ, R_{b}, Ar₁, Ar₂, C1 and C2 are the same as defined with respect to Formula 1 and Formula 1-a.

25. The amine compound of claim 22, wherein the amine compound represented by Formula 1 is represented by any one selected from among the following Formula 5-1 to Formula 5-3: 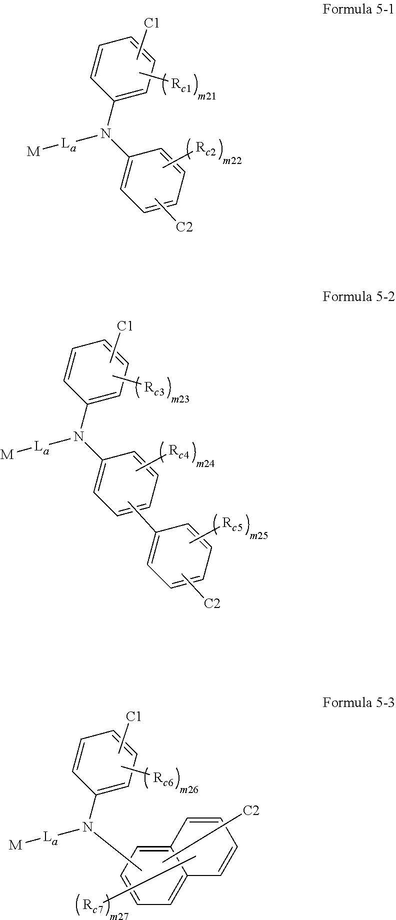
in Formula 5-1 to Formula 5-3,
R_{c1} to R_{c7} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m21 to m26 are each independently an integer of 0 to 4,
m27 is an integer of 0 to 6, and
M, Lₐ, C1, and C2 are the same as defined with respect to Formula 1.

26. The amine compound of claim 22, wherein the amine compound represented by Formula 1 is represented by any one selected from among the following Formula 8-1 to Formula 8-5: 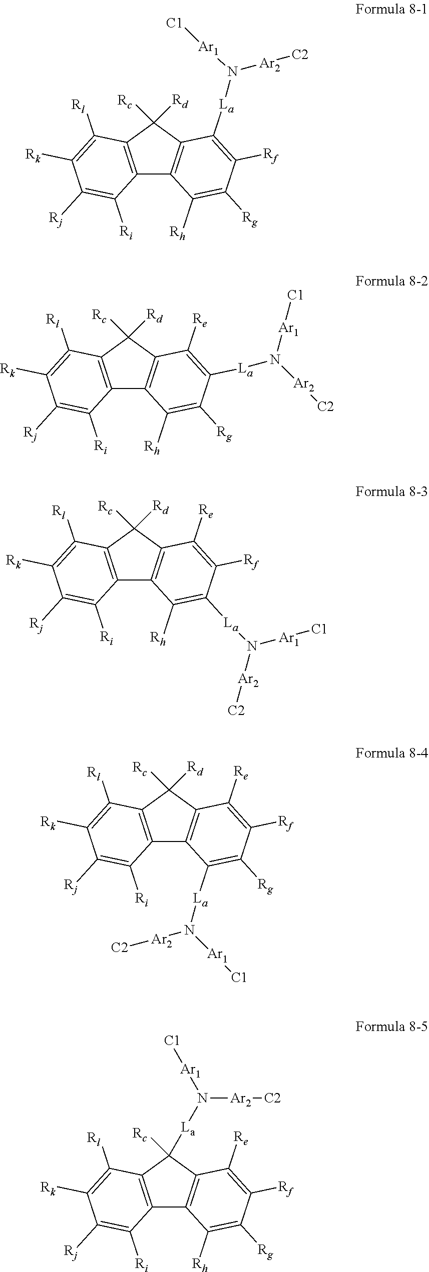
in Formula 8-1 to Formula 8-5,
at least one among C1 and C2 is a substituted or unsubstituted bicycloheptanyl group, and
Ar₁, Ar₂, Lₐ, C1, C2, and R_{c} to Rₗ are the same as defined with respect to Formula 1 and Formula 1-b.

27. The amine compound of claim 22, wherein the amine compound represented by Formula 1 is represented by any one selected from among the following Formula 9-1 to Formula 9-4: 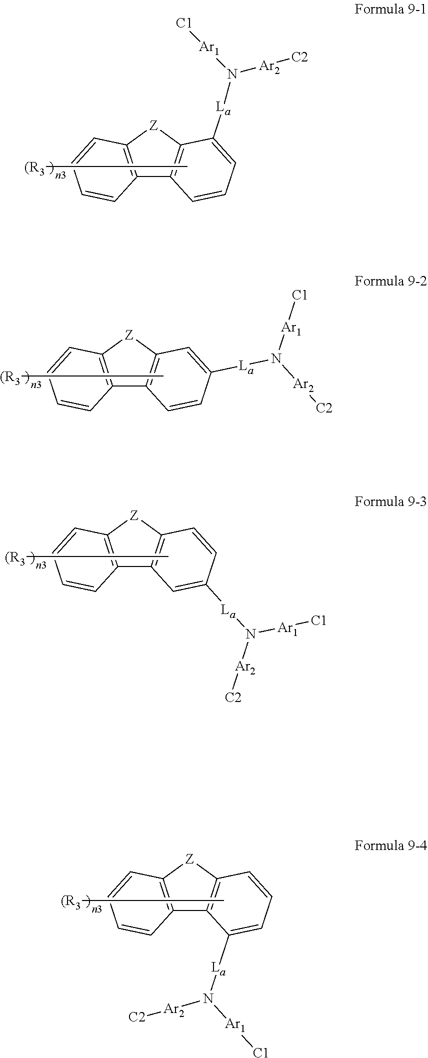
in Formula 9-1 to Formula 9-4,
at least one among C1 and C2 is a substituted or unsubstituted bicycloheptanyl group, and
Z, R₃, Ar₁, Ar₂, Lₐ, n3, C1, and C2 are the same as defined with respect to Formula 1 and Formula 1-c.

28. The amine compound of claim 22, wherein the amine compound is at least one selected from compounds in the following Compound Group 1: 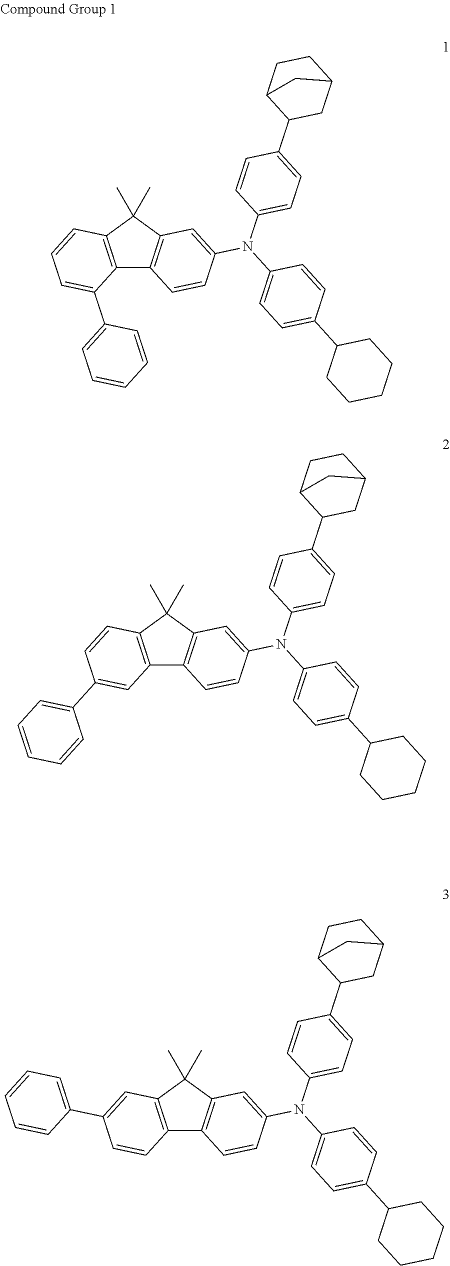 [SMILES: CC1(C)c2cc(N(c3ccc(C4CCCCC4)cc3)c3ccc(C4CC5CCC4C5)cc3)ccc2-c2c(-c3ccccc3)cccc21 ; CC1(C)c2ccc(-c3ccccc3)cc2-c2ccc(N(c3ccc(C4CCCCC4)cc3)c3ccc(C4CC5CCC4C5)cc3)cc21 ; CC1(C)c2cc(-c3ccccc3)ccc2-c2ccc(N(c3ccc(C4CCCCC4)cc3)c3ccc(C4CC5CCC4C5)cc3)cc21] 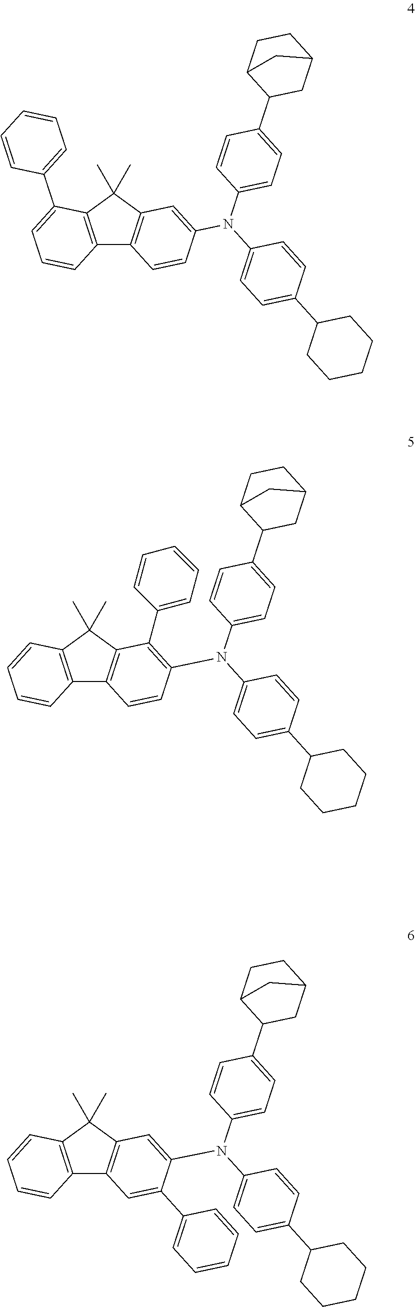 [SMILES: CC1(C)c2cc(N(c3ccc(C4CCCCC4)cc3)c3ccc(C4CC5CCC4C5)cc3)ccc2-c2cccc(-c3ccccc3)c21 ; CC1(C)c2ccccc2-c2ccc(N(c3ccc(C4CCCCC4)cc3)c3ccc(C4CC5CCC4C5)cc3)c(-c3ccccc3)c21 ; CC1(C)c2ccccc2-c2cc(-c3ccccc3)c(N(c3ccc(C4CCCCC4)cc3)c3ccc(C4CC5CCC4C5)cc3)cc21] 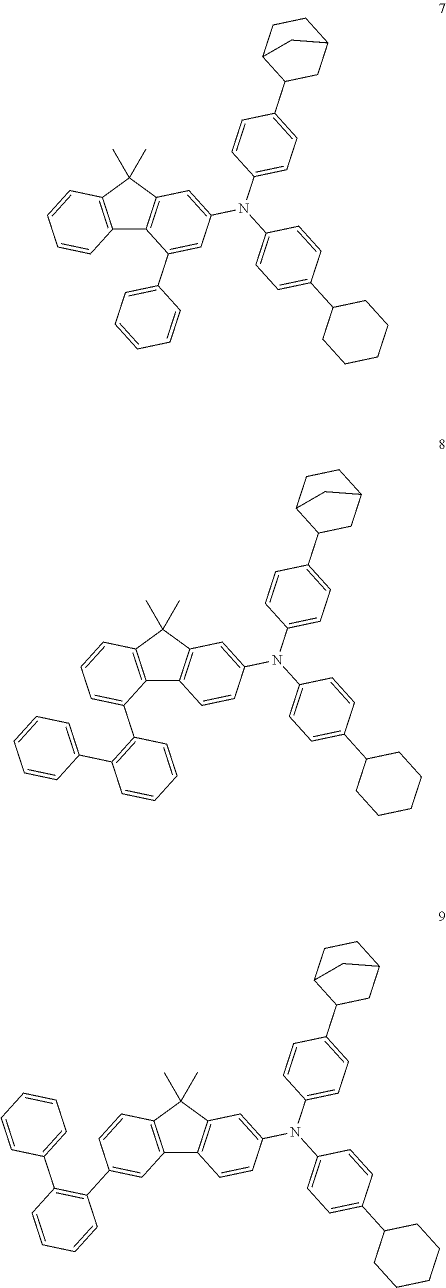 [SMILES: CC1(C)c2ccccc2-c2c(-c3ccccc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4CC5CCC4C5)cc3)cc21] 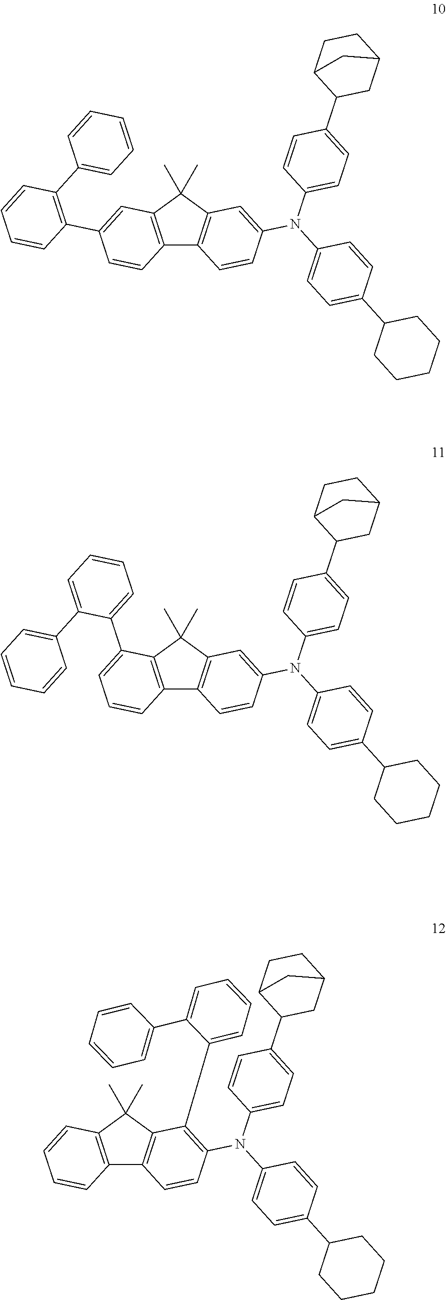 [SMILES: CC1(C)c2cc(-c3ccccc3-c3ccccc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4C5)cc3)c(-c3ccccc3-c3ccccc3)c21] 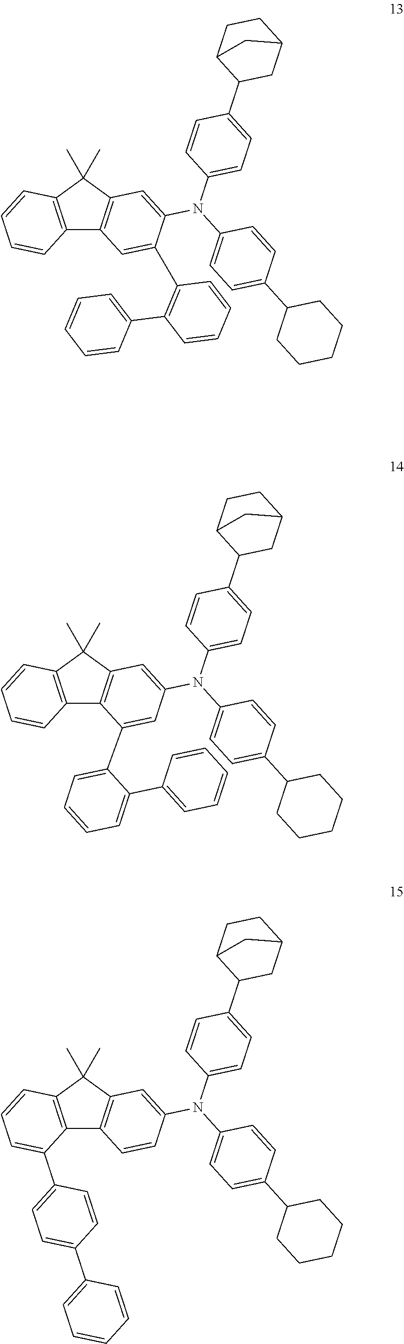 [SMILES: CC1(C)c2ccccc2-c2cc(-c3ccccc3-c3ccccc3)c(N(c3ccc(C4CCCCC4)cc3)c3ccc(C4CC5CCC4C5)cc3)cc21 ; CC1(C)c2ccccc2-c2c(-c3ccccc3-c3ccccc3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(-c4ccccc4)cc3)cccc21] 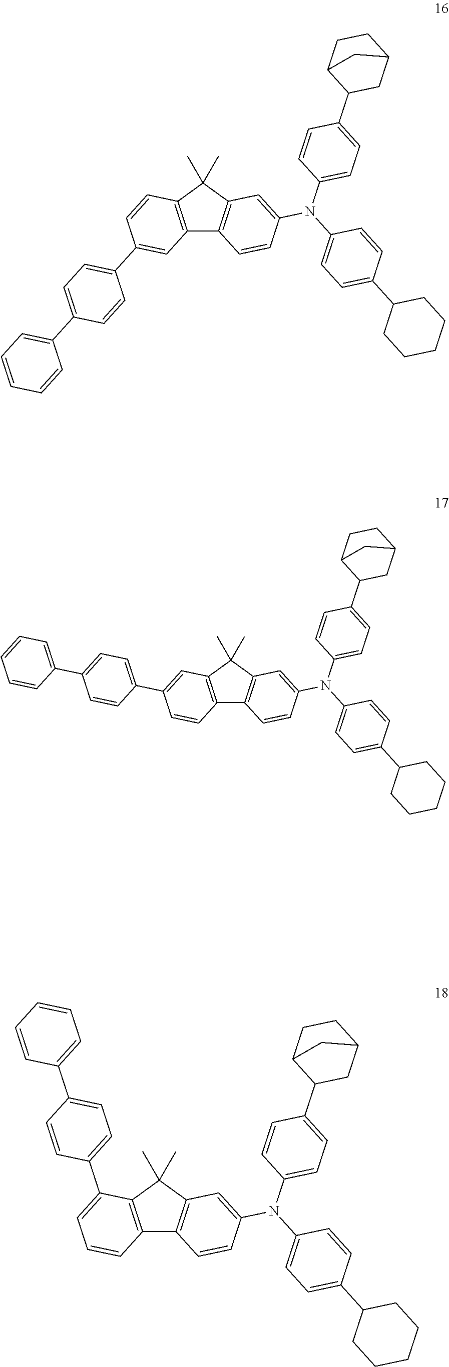 [SMILES: CC1(C)c2ccc(-c3ccc(-c4ccccc4)cc3)cc2-c2ccc(N(c3ccc(C4CCCCC4)cc3)c3ccc(C4CC5CCC4C5)cc3)cc21 ; CC1(C)c2cc(-c3ccc(-c4ccccc4)cc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc(-c4ccccc4)cc3)c21] 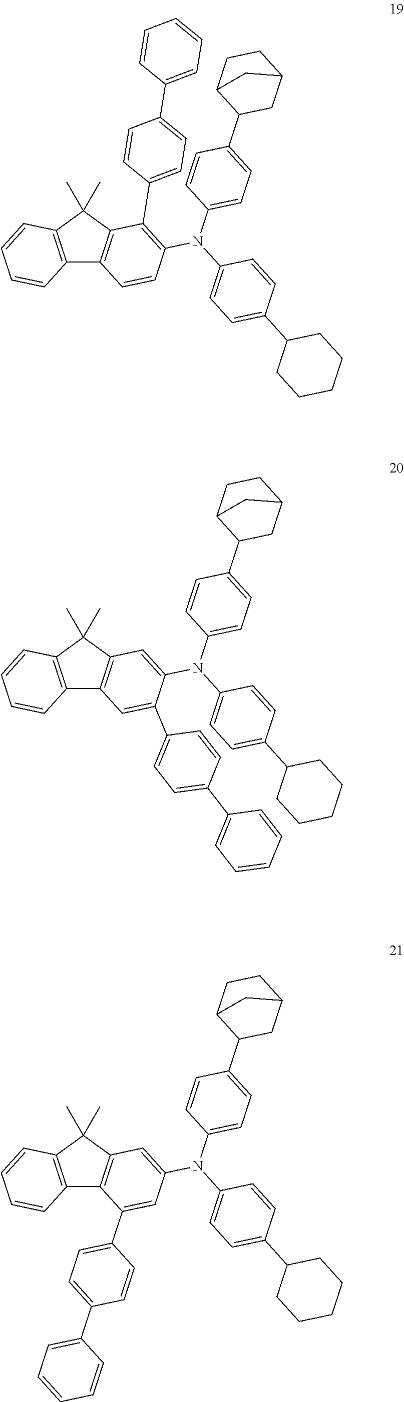 [SMILES: CC1(C)c2ccccc2-c2ccc(N(c3ccc(C4CCCCC4)cc3)c3ccc(C4CC5CCC4C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4CC5CCC4C5)cc3)cc21 ; CC1(C)c2ccccc2-c2c(-c3ccc(-c4ccccc4)cc3)cc(N(c3ccc(C4CCCCC4)cc3)c3ccc(C4CC5CCC4C5)cc3)cc21] 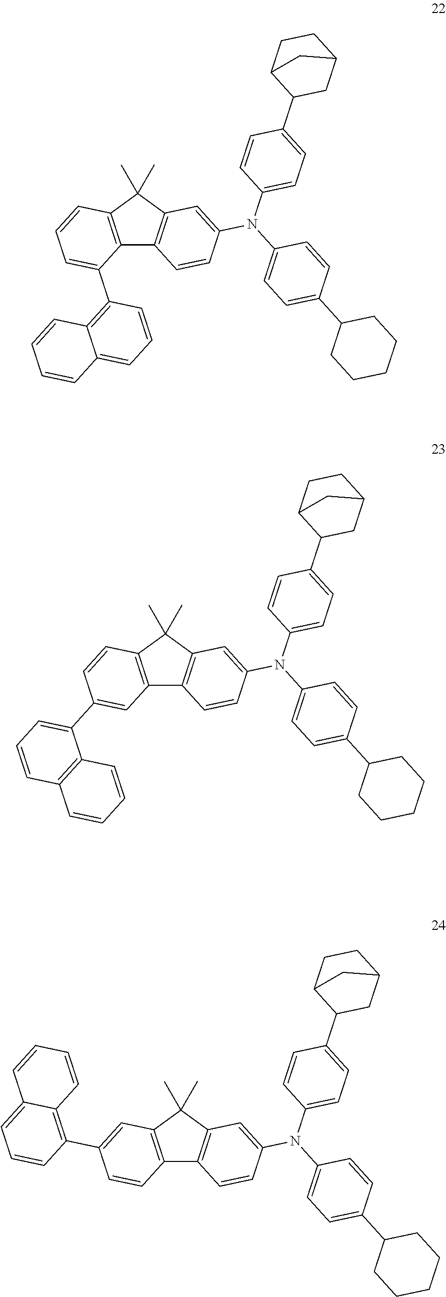 [SMILES: CC1(C)c2cc(N(c3ccc(C4CCCCC4)cc3)c3ccc(C4CC5CCC4C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CC5CCC4C5)cc3)cc21 ; CC1(C)c2cc(-c3cccc4ccccc34)ccc2-c2ccc(N(c3ccc(C4CCCCC4)cc3)c3ccc(C4CC5CCC4C5)cc3)cc21] 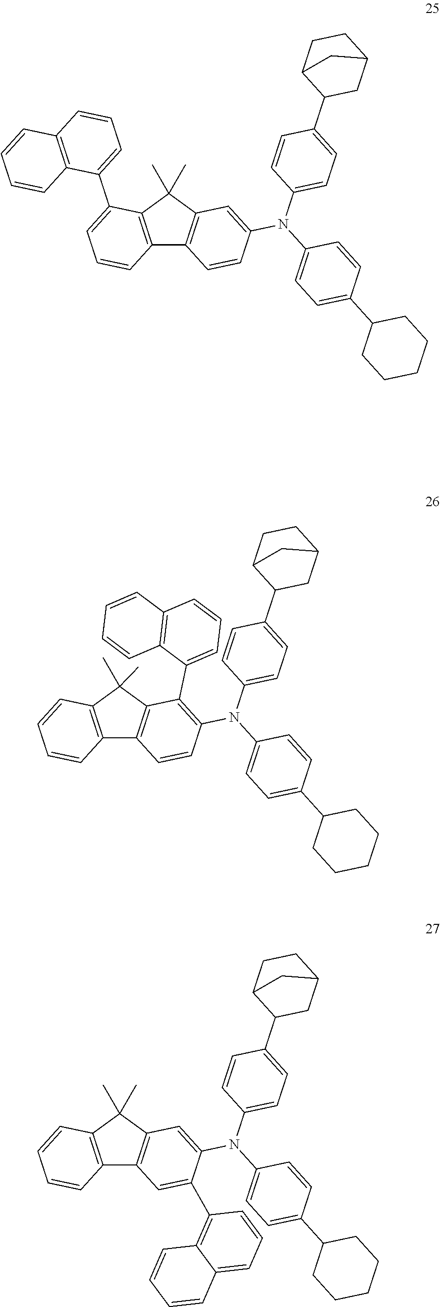 [SMILES: CC1(C)c2cc(N(c3ccc(C4CCCCC4)cc3)c3ccc(C4CC5CCC4C5)cc3)ccc2-c2cccc(-c3cccc4ccccc34)c21 ; CC1(C)c2ccccc2-c2ccc(N(c3ccc(C4CCCCC4)cc3)c3ccc(C4CC5CCC4C5)cc3)c(-c3cccc4ccccc34)c21 ; CC1(C)c2ccccc2-c2cc(-c3cccc4ccccc34)c(N(c3ccc(C4CCCCC4)cc3)c3ccc(C4CC5CCC4C5)cc3)cc21] 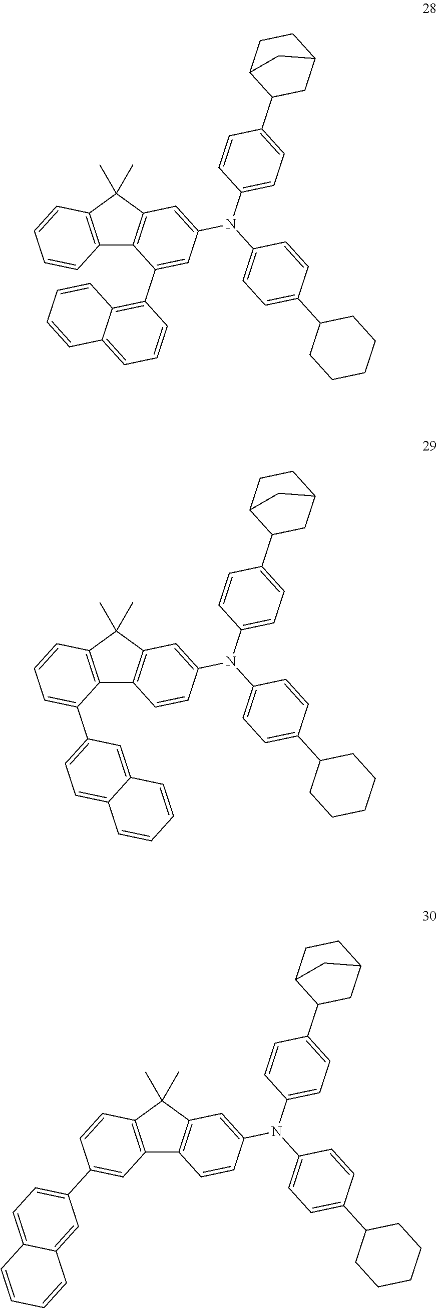 [SMILES: CC1(C)c2ccccc2-c2c(-c3cccc4ccccc34)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CC5CCC4C5)cc3)cc21] 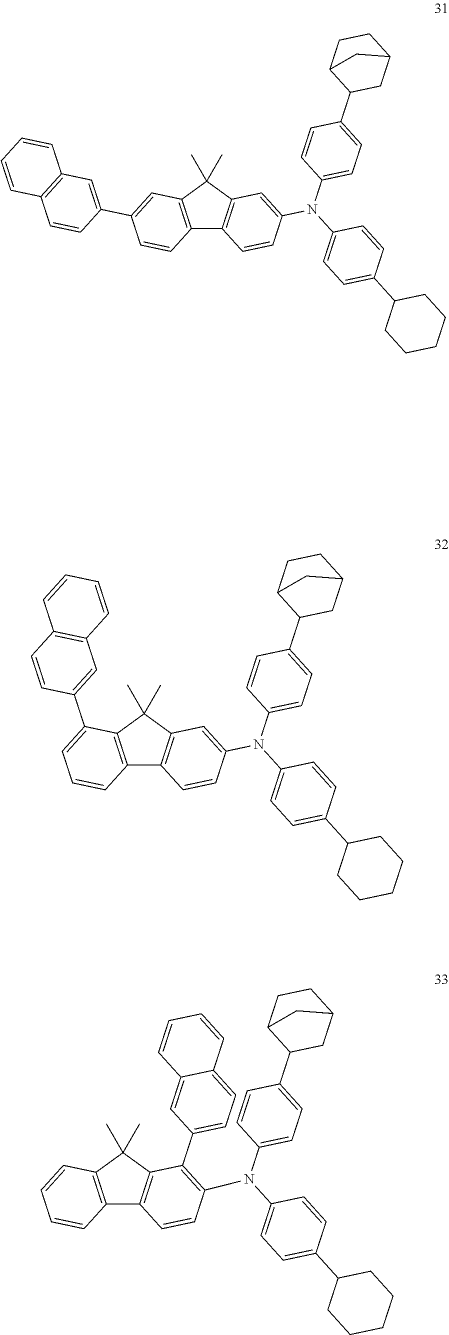 [SMILES: CC1(C)c2cc(-c3ccc4ccccc4c3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc4ccccc4c3)c21 ; CC1(C)c2ccccc2-c2ccc(N(c3ccc(C4CCCCC4)cc3)c3ccc(C4CC5CCC4C5)cc3)c(-c3ccc4ccccc4c3)c21] 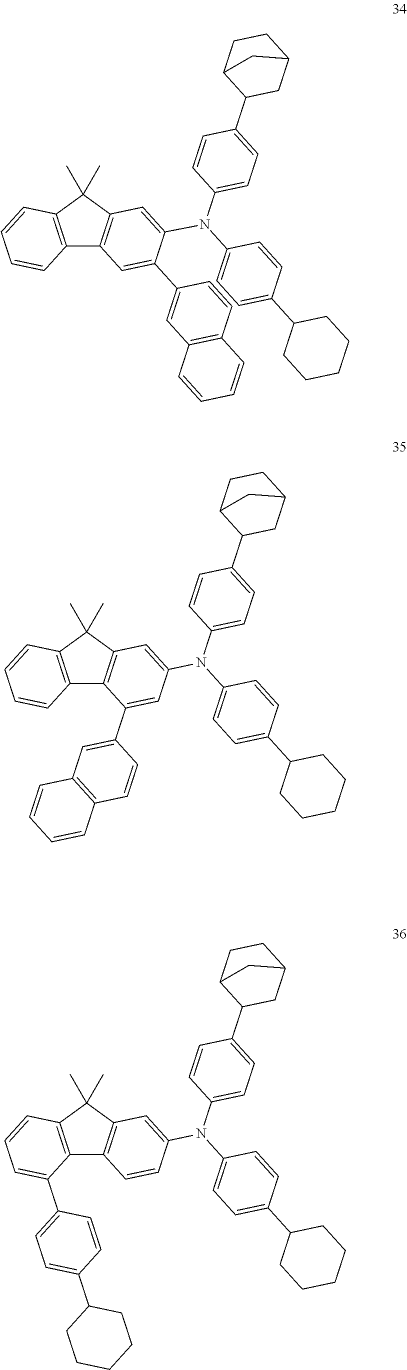 [SMILES: CC1(C)c2ccccc2-c2cc(-c3ccc4ccccc4c3)c(N(c3ccc(C4CCCCC4)cc3)c3ccc(C4CC5CCC4C5)cc3)cc21 ; CC1(C)c2ccccc2-c2c(-c3ccc4ccccc4c3)cc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2c(-c3ccc(C4CCCCC4)cc3)cccc21] 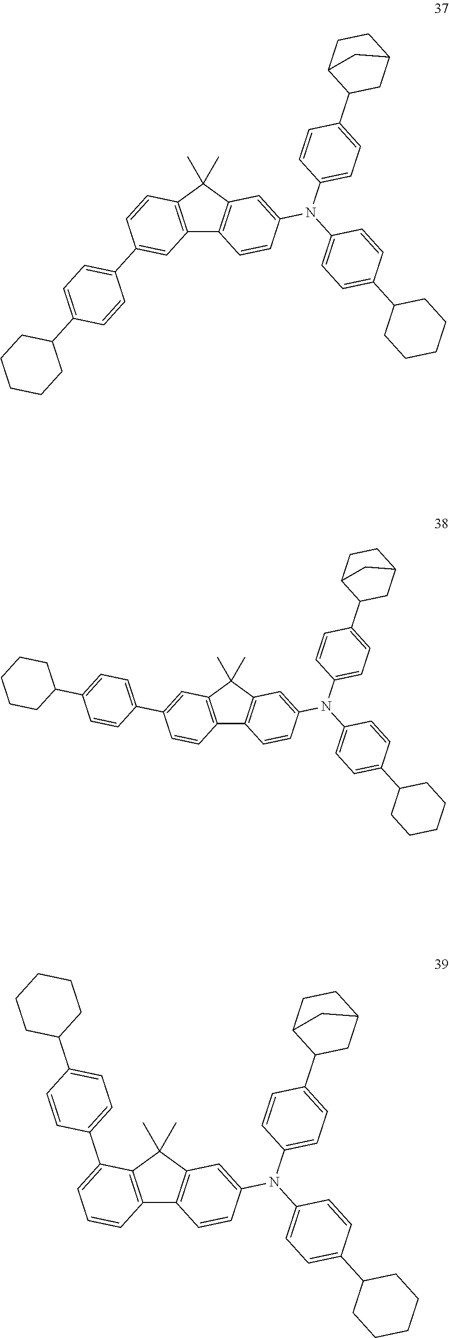 [SMILES: CC1(C)c2ccc(-c3ccc(C4CCCCC4)cc3)cc2-c2ccc(N(c3ccc(C4CCCCC4)cc3)c3ccc(C4CC5CCC4C5)cc3)cc21 ; CC1(C)c2cc(-c3ccc(C4CCCCC4)cc3)ccc2-c2ccc(N(c3ccc(C4CCCCC4)cc3)c3ccc(C4CC5CCC4C5)cc3)cc21 ; CC1(C)c2cc(N(c3ccc(C4CCCCC4)cc3)c3ccc(C4CC5CCC4C5)cc3)ccc2-c2cccc(-c3ccc(C4CCCCC4)cc3)c21] 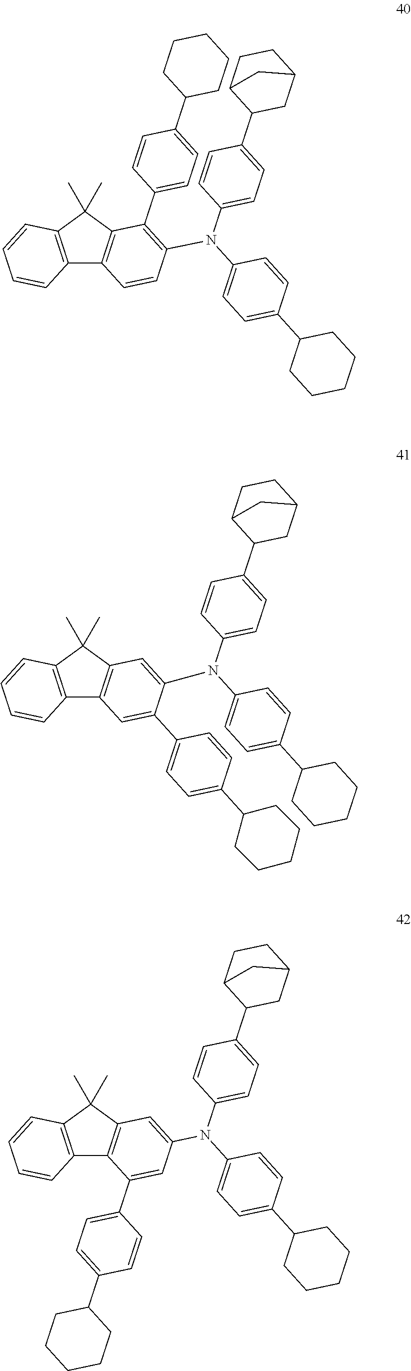 [SMILES: CC1(C)c2ccccc2-c2ccc(N(c3ccc(C4CCCCC4)cc3)c3ccc(C4CC5CCC4C5)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C4CC5CCC4C5)cc3)cc21 ; CC1(C)c2ccccc2-c2c(-c3ccc(C4CCCCC4)cc3)cc(N(c3ccc(C4CCCCC4)cc3)c3ccc(C4CC5CCC4C5)cc3)cc21] 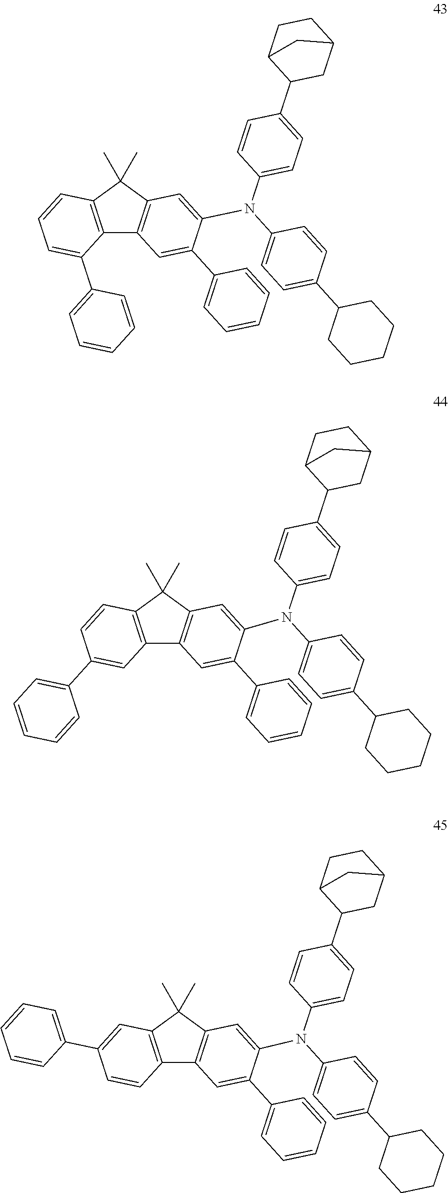 [SMILES: CC1(C)c2cc(N(c3ccc(C4CCCCC4)cc3)c3ccc(C4CC5CCC4C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4CC5CCC4C5)cc3)cc21 ; CC1(C)c2cc(-c3ccccc3)ccc2-c2cc(-c3ccccc3)c(N(c3ccc(C4CCCCC4)cc3)c3ccc(C4CC5CCC4C5)cc3)cc21] 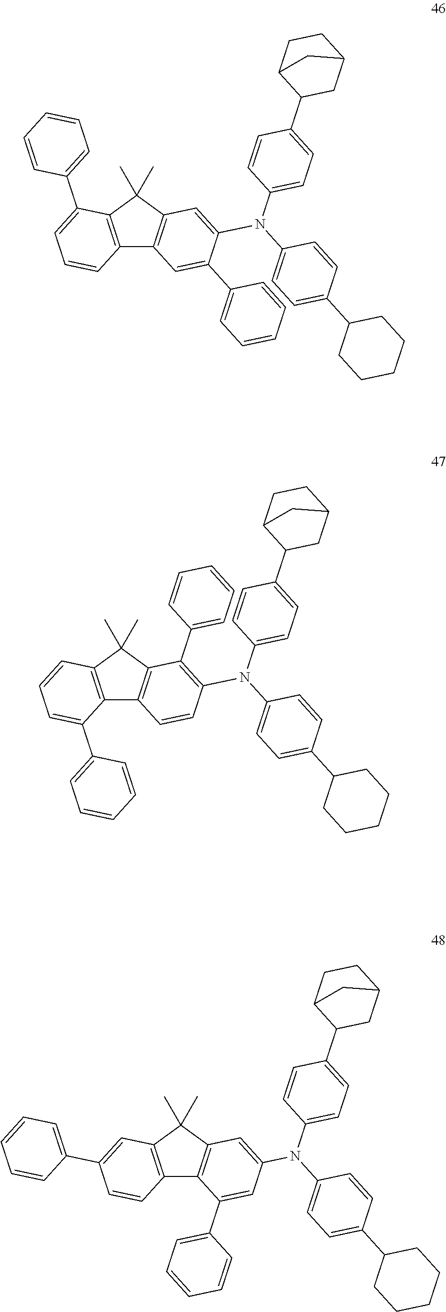 [SMILES: CC1(C)c2cc(N(c3ccc(C4CCCCC4)cc3)c3ccc(C4CC5CCC4C5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4CC5CCC4C5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4CC5CCC4C5)cc3)cc21] 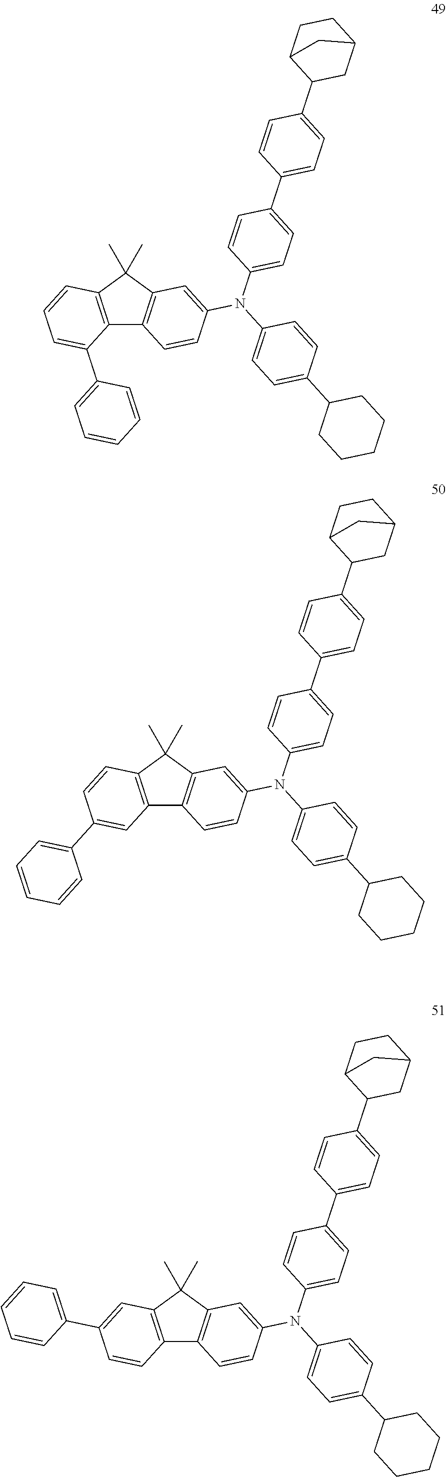 [SMILES: CC1(C)c2cc(N(c3ccc(-c4ccc(C5CC6CCC5C6)cc4)cc3)c3ccc(C4CCCCC4)cc3)ccc2-c2c(-c3ccccc3)cccc21 ; CC1(C)c2ccc(-c3ccccc3)cc2-c2ccc(N(c3ccc(-c4ccc(C5CC6CCC5C6)cc4)cc3)c3ccc(C4CCCCC4)cc3)cc21 ; CC1(C)c2cc(-c3ccccc3)ccc2-c2ccc(N(c3ccc(-c4ccc(C5CC6CCC5C6)cc4)cc3)c3ccc(C4CCCCC4)cc3)cc21] 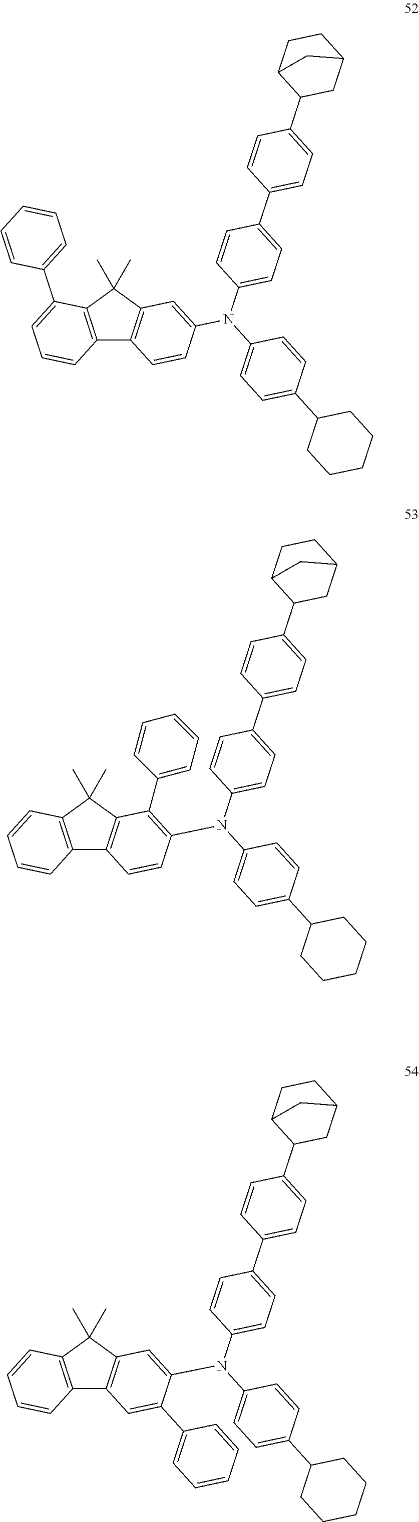 [SMILES: CC1(C)c2cc(N(c3ccc(-c4ccc(C5CC6CCC5C6)cc4)cc3)c3ccc(C4CCCCC4)cc3)ccc2-c2cccc(-c3ccccc3)c21 ; CC1(C)c2ccccc2-c2ccc(N(c3ccc(-c4ccc(C5CC6CCC5C6)cc4)cc3)c3ccc(C4CCCCC4)cc3)c(-c3ccccc3)c21 ; CC1(C)c2ccccc2-c2cc(-c3ccccc3)c(N(c3ccc(-c4ccc(C5CC6CCC5C6)cc4)cc3)c3ccc(C4CCCCC4)cc3)cc21] 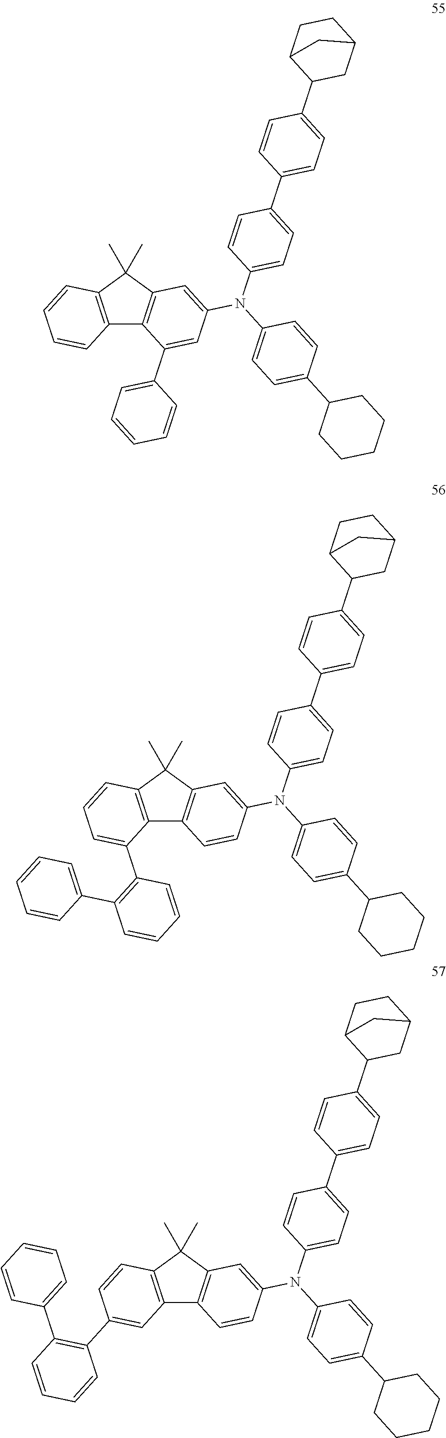 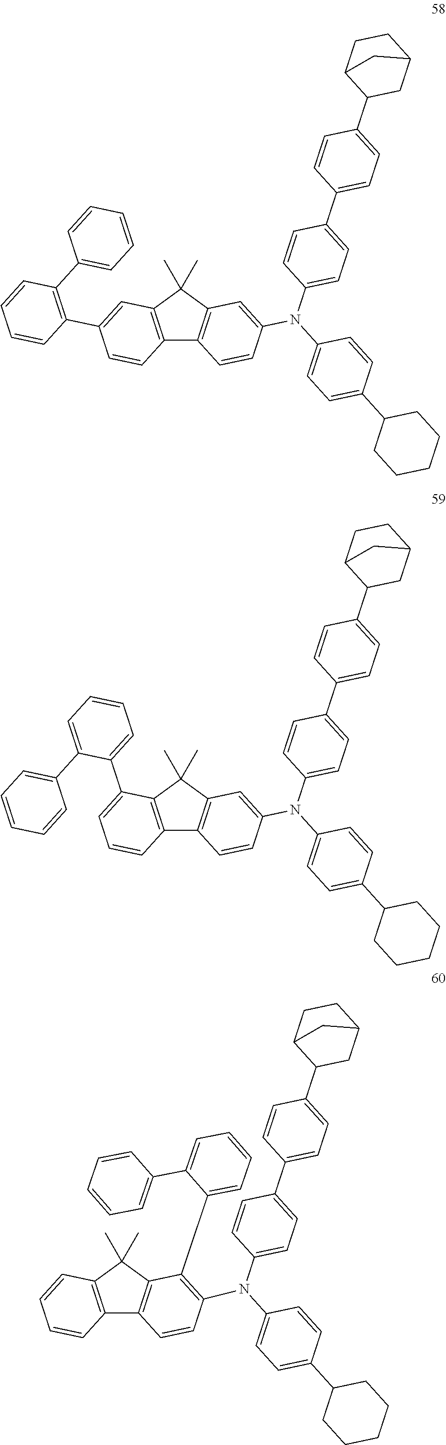 [SMILES: CC1(C)c2cc(-c3ccccc3-c3ccccc3)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3ccccc3-c3ccccc3)c21 ; CC1(C)c2ccccc2-c2ccc(N(c3ccc(-c4ccc(C5CC6CCC5C6)cc4)cc3)c3ccc(C4CCCCC4)cc3)c(-c3ccccc3-c3ccccc3)c21] 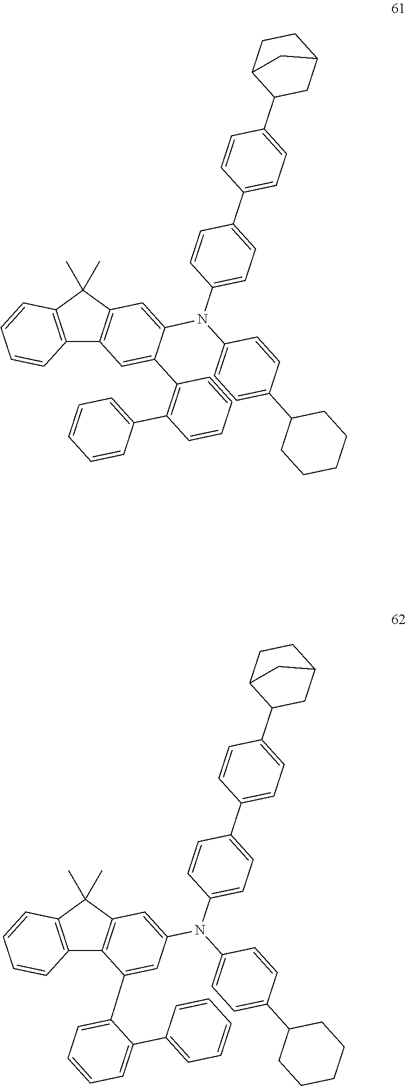 [SMILES: CC1(C)c2ccccc2-c2cc(-c3ccccc3-c3ccccc3)c(N(c3ccc(-c4ccc(C5CC6CCC5C6)cc4)cc3)c3ccc(C4CCCCC4)cc3)cc21 ; CC1(C)c2ccccc2-c2c(-c3ccccc3-c3ccccc3)cc(N(c3ccc(-c4ccc(C5CC6CCC5C6)cc4)cc3)c3ccc(C4CCCCC4)cc3)cc21] 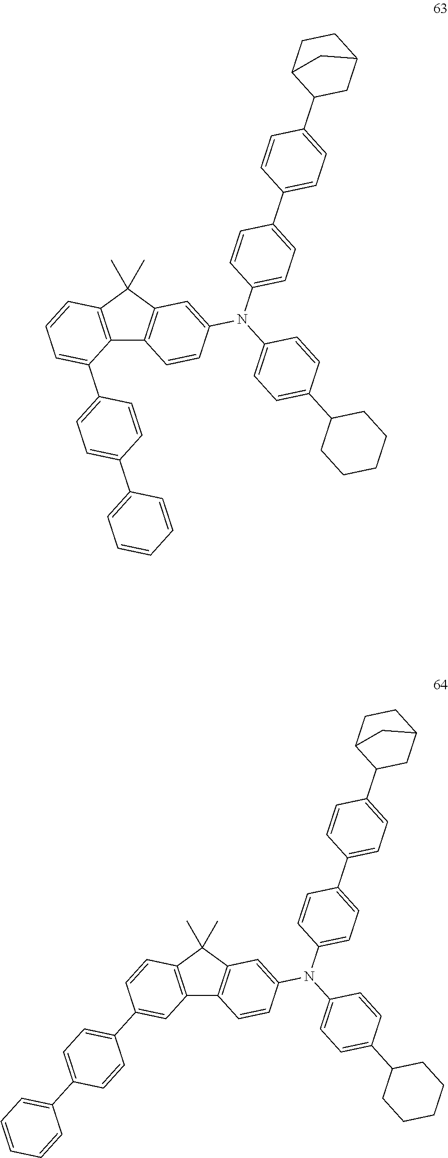 [SMILES: CC1(C)c2cc(N(c3ccc(-c4ccc(C5CC6CCC5C6)cc4)cc3)c3ccc(C4CCCCC4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(-c4ccc(C5CC6CCC5C6)cc4)cc3)c3ccc(C4CCCCC4)cc3)cc21] 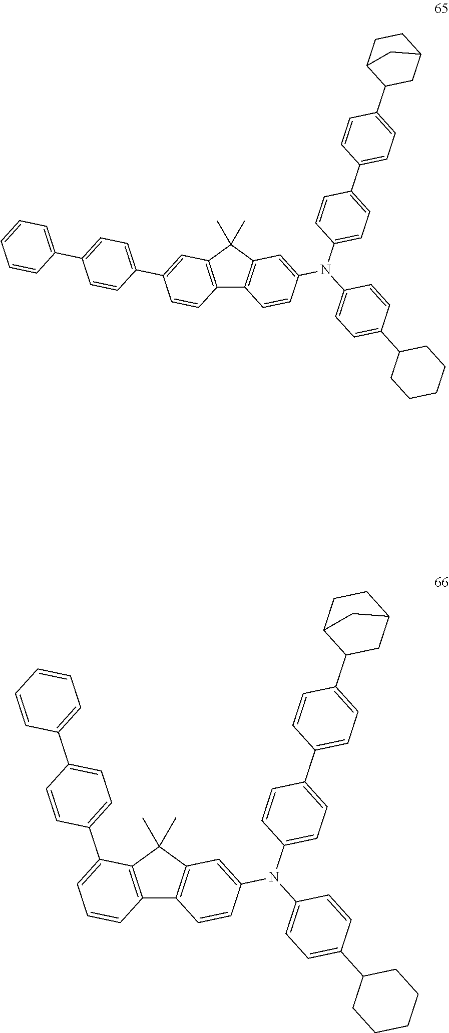 [SMILES: CC1(C)c2cc(-c3ccc(-c4ccccc4)cc3)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3ccc(-c4ccccc4)cc3)c21] 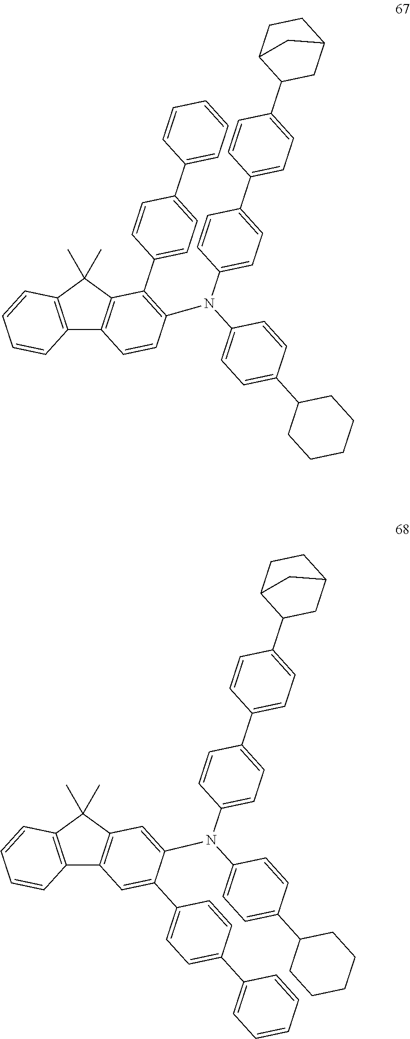 [SMILES: CC1(C)c2ccccc2-c2ccc(N(c3ccc(-c4ccc(C5CC6CCC5C6)cc4)cc3)c3ccc(C4CCCCC4)cc3)c(-c3ccc(-c4ccccc4)cc3)c21 ; CC1(C)c2ccccc2-c2cc(-c3ccc(-c4ccccc4)cc3)c(N(c3ccc(-c4ccc(C5CC6CCC5C6)cc4)cc3)c3ccc(C4CCCCC4)cc3)cc21] 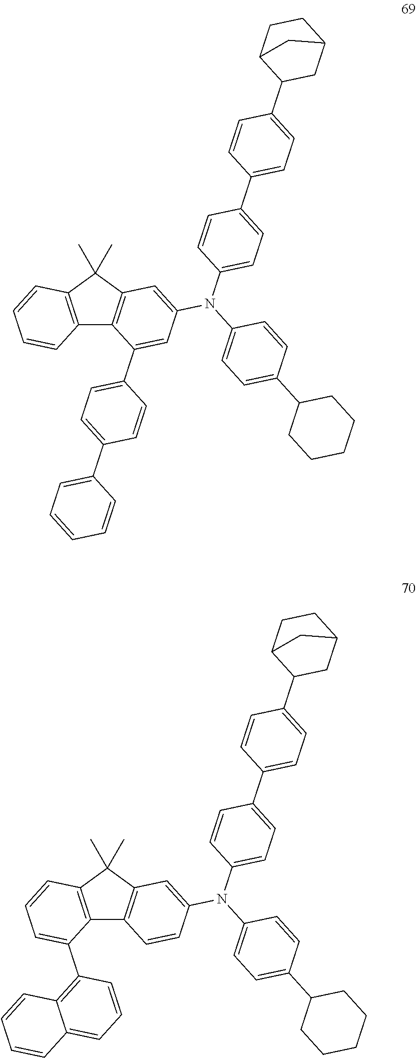 [SMILES: CC1(C)c2ccccc2-c2c(-c3ccc(-c4ccccc4)cc3)cc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2c(-c3cccc4ccccc34)cccc21] 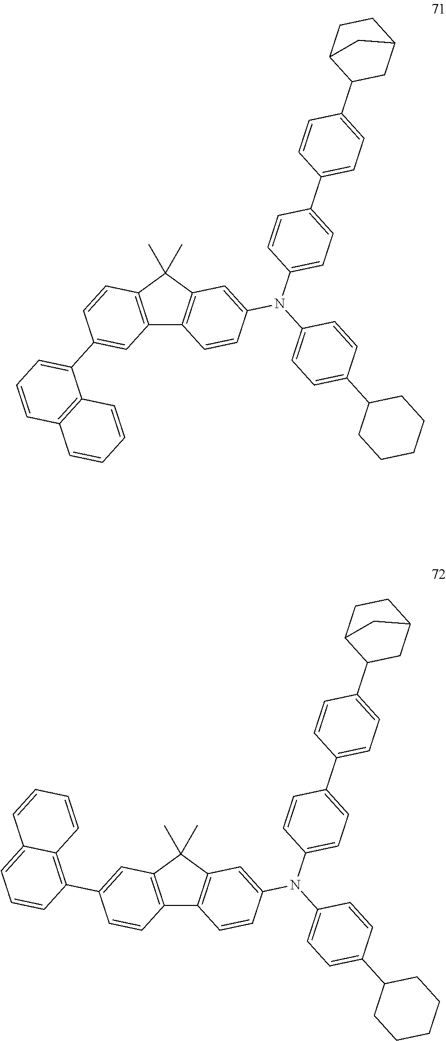 [SMILES: CC1(C)c2ccc(-c3cccc4ccccc34)cc2-c2ccc(N(c3ccc(-c4ccc(C5CC6CCC5C6)cc4)cc3)c3ccc(C4CCCCC4)cc3)cc21 ; CC1(C)c2cc(-c3cccc4ccccc34)ccc2-c2ccc(N(c3ccc(-c4ccc(C5CC6CCC5C6)cc4)cc3)c3ccc(C4CCCCC4)cc3)cc21] 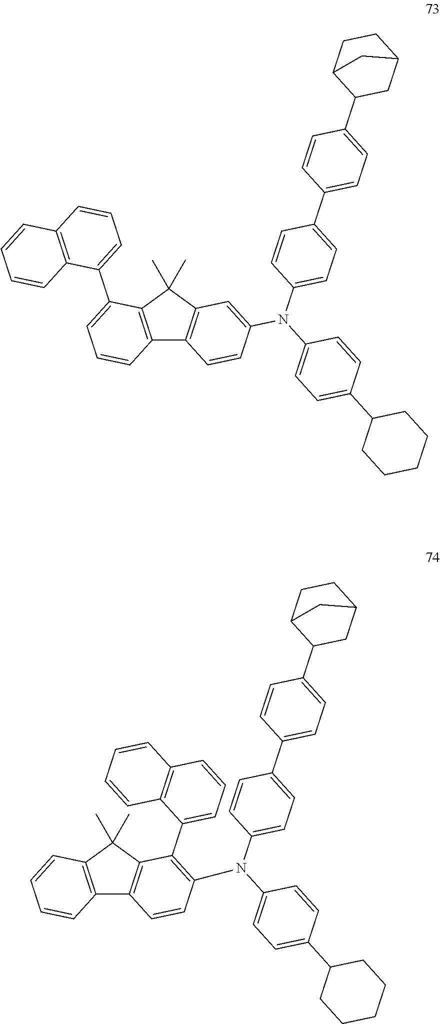 [SMILES: CC1(C)c2cc(N(c3ccc(-c4ccc(C5CC6CCC5C6)cc4)cc3)c3ccc(C4CCCCC4)cc3)ccc2-c2cccc(-c3cccc4ccccc34)c21 ; CC1(C)c2ccccc2-c2ccc(N(c3ccc(-c4ccc(C5CC6CCC5C6)cc4)cc3)c3ccc(C4CCCCC4)cc3)c(-c3cccc4ccccc34)c21] 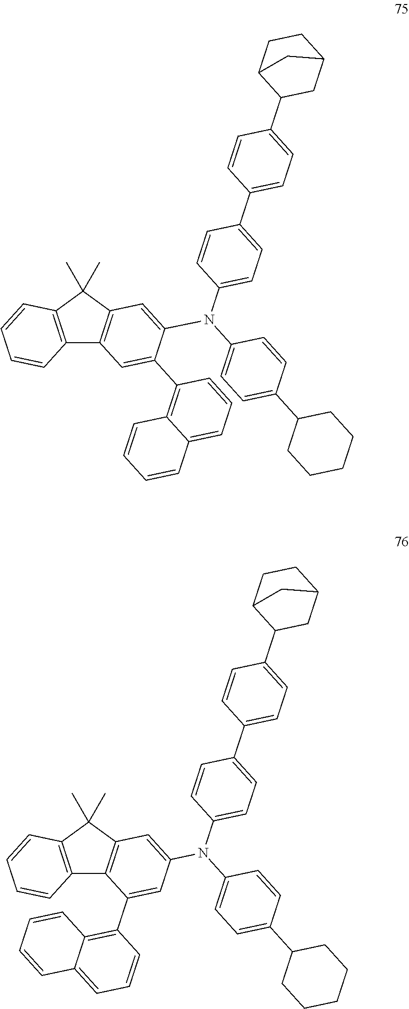 [SMILES: CC1(C)c2ccccc2-c2cc(-c3cccc4ccccc34)c(N(c3ccc(-c4ccc(C5CC6CCC5C6)cc4)cc3)c3ccc(C4CCCCC4)cc3)cc21 ; CC1(C)c2ccccc2-c2c(-c3cccc4ccccc34)cc(N(c3ccc(-c4ccc(C5CC6CCC5C6)cc4)cc3)c3ccc(C4CCCCC4)cc3)cc21] 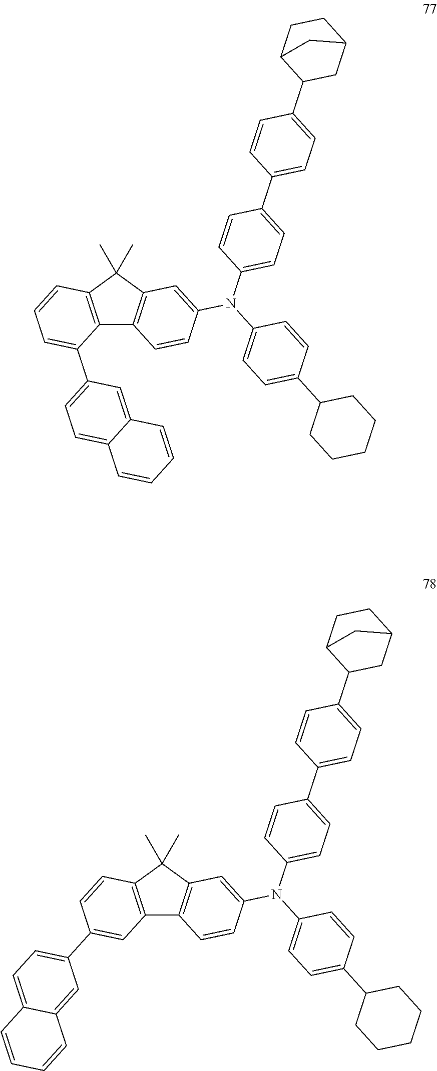 [SMILES: CC1(C)c2cc(N(c3ccc(-c4ccc(C5CC6CCC5C6)cc4)cc3)c3ccc(C4CCCCC4)cc3)ccc2-c2c(-c3ccc4ccccc4c3)cccc21 ; CC1(C)c2ccc(-c3ccc4ccccc4c3)cc2-c2ccc(N(c3ccc(-c4ccc(C5CC6CCC5C6)cc4)cc3)c3ccc(C4CCCCC4)cc3)cc21] 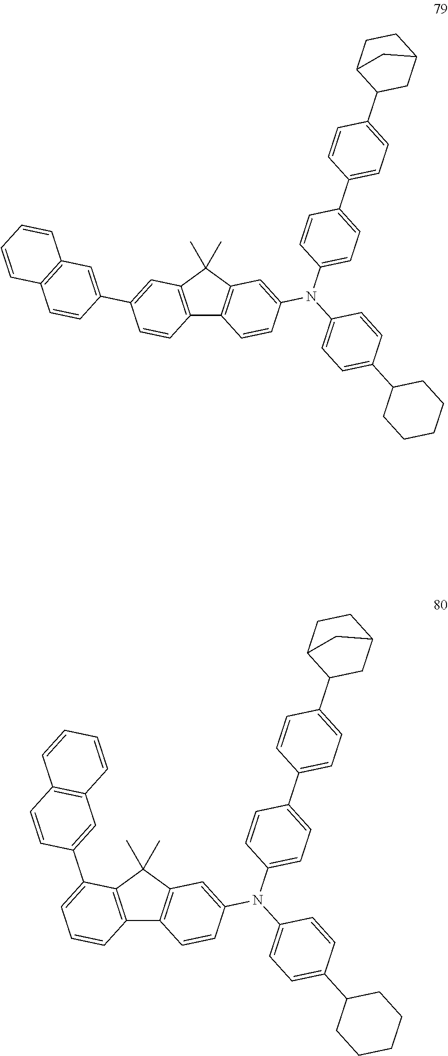 [SMILES: CC1(C)c2cc(-c3ccc4ccccc4c3)ccc2-c2ccc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2cccc(-c3ccc4ccccc4c3)c21] 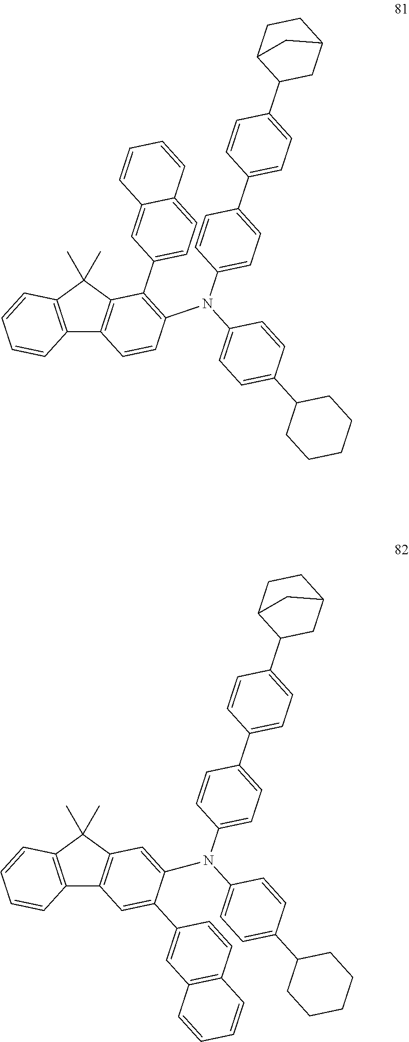 [SMILES: CC1(C)c2ccccc2-c2ccc(N(c3ccc(-c4ccc(C5CC6CCC5C6)cc4)cc3)c3ccc(C4CCCCC4)cc3)c(-c3ccc4ccccc4c3)c21 ; CC1(C)c2ccccc2-c2cc(-c3ccc4ccccc4c3)c(N(c3ccc(-c4ccc(C5CC6CCC5C6)cc4)cc3)c3ccc(C4CCCCC4)cc3)cc21] 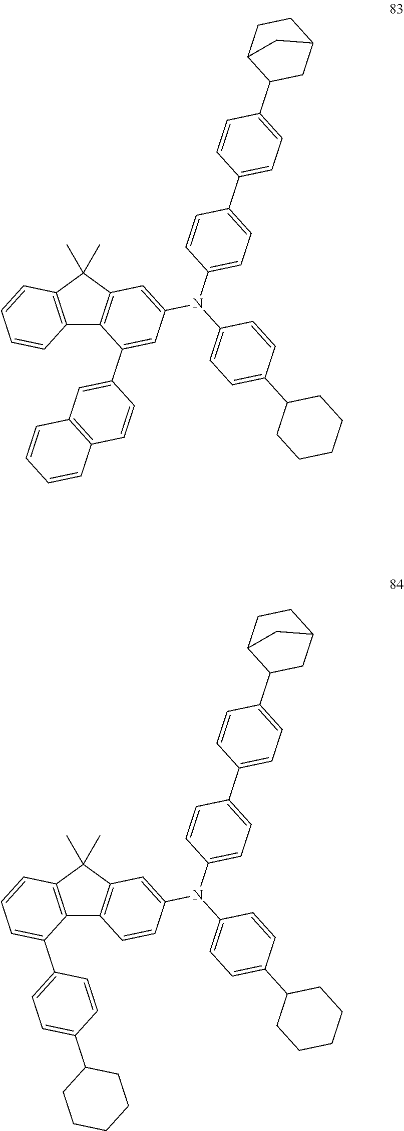 [SMILES: CC1(C)c2ccccc2-c2c(-c3ccc4ccccc4c3)cc(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)ccc2-c2c(-c3ccc(C4CCCCC4)cc3)cccc21] 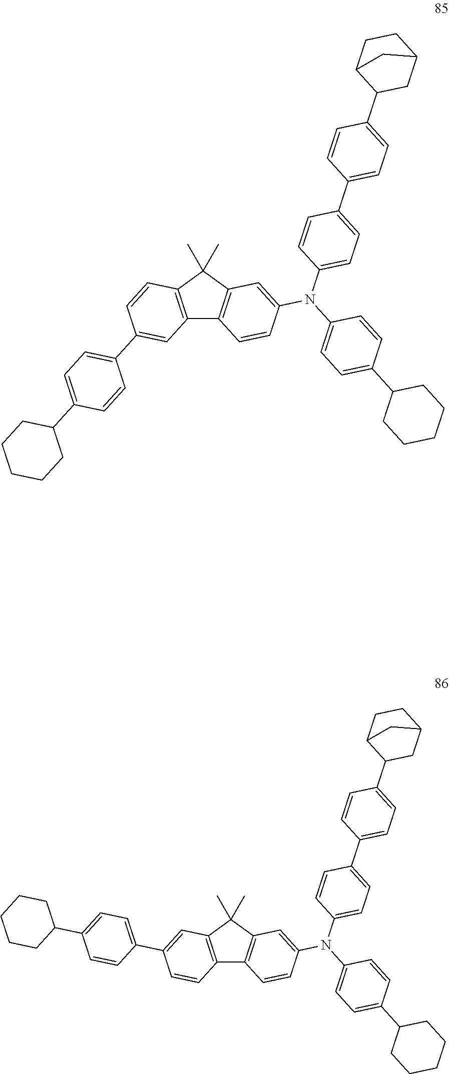 [SMILES: CC1(C)c2ccc(-c3ccc(C4CCCCC4)cc3)cc2-c2ccc(N(c3ccc(-c4ccc(C5CC6CCC5C6)cc4)cc3)c3ccc(C4CCCCC4)cc3)cc21 ; CC1(C)c2cc(-c3ccc(C4CCCCC4)cc3)ccc2-c2ccc(N(c3ccc(-c4ccc(C5CC6CCC5C6)cc4)cc3)c3ccc(C4CCCCC4)cc3)cc21] 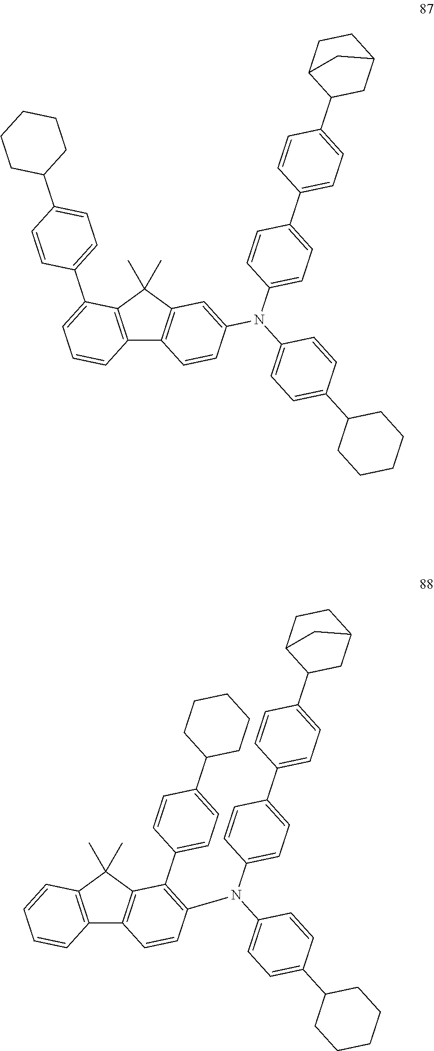 [SMILES: CC1(C)c2cc(N(c3ccc(-c4ccc(C5CC6CCC5C6)cc4)cc3)c3ccc(C4CCCCC4)cc3)ccc2-c2cccc(-c3ccc(C4CCCCC4)cc3)c21 ; CC1(C)c2ccccc2-c2ccc(N(c3ccc(-c4ccc(C5CC6CCC5C6)cc4)cc3)c3ccc(C4CCCCC4)cc3)c(-c3ccc(C4CCCCC4)cc3)c21] 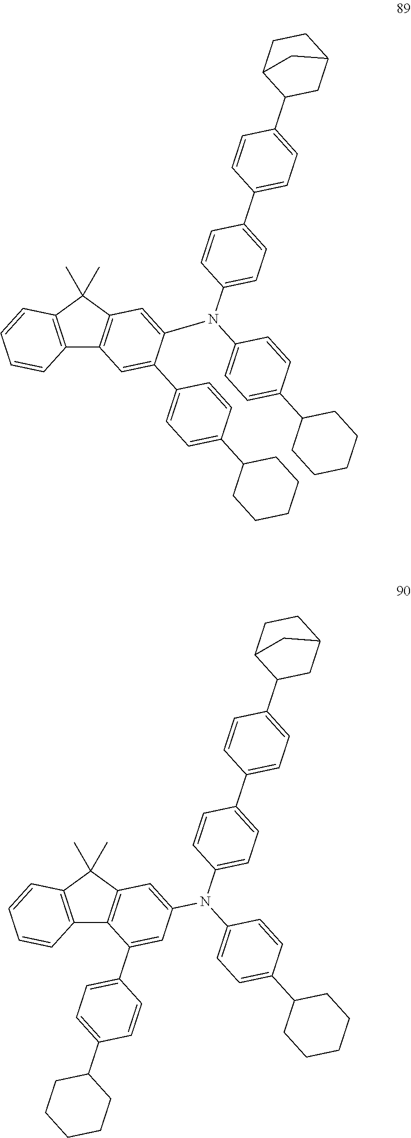 [SMILES: CC1(C)c2ccccc2-c2cc(-c3ccc(C4CCCCC4)cc3)c(N(c3ccc(-c4ccc(C5CC6CCC5C6)cc4)cc3)c3ccc(C4CCCCC4)cc3)cc21 ; CC1(C)c2ccccc2-c2c(-c3ccc(C4CCCCC4)cc3)cc(N(c3ccc(-c4ccc(C5CC6CCC5C6)cc4)cc3)c3ccc(C4CCCCC4)cc3)cc21] 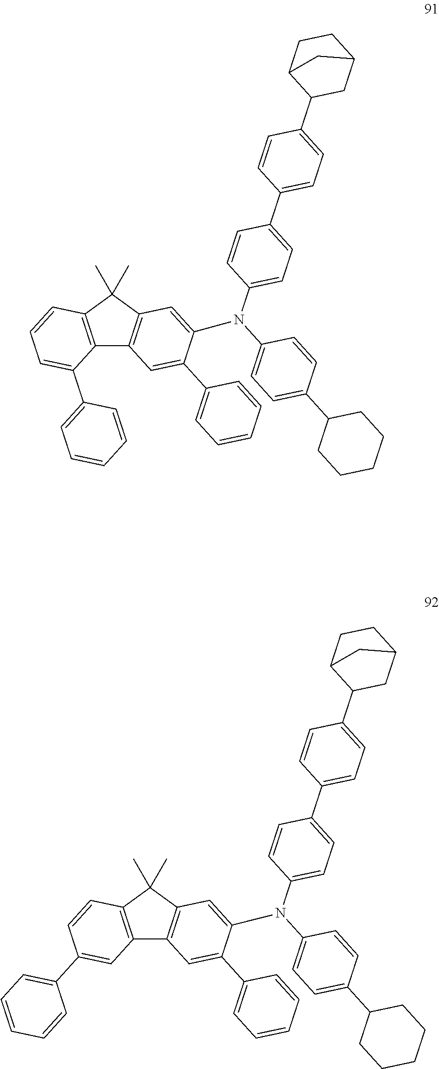 [SMILES: CC1(C)c2cc(N(c3ccc(-c4ccc(C5CC6CCC5C6)cc4)cc3)c3ccc(C4CCCCC4)cc3)c(-c3ccccc3)cc2-c2c(-c3ccccc3)cccc21 ; CC1(C)c2ccc(-c3ccccc3)cc2-c2cc(-c3ccccc3)c(N(c3ccc(-c4ccc(C5CC6CCC5C6)cc4)cc3)c3ccc(C4CCCCC4)cc3)cc21] 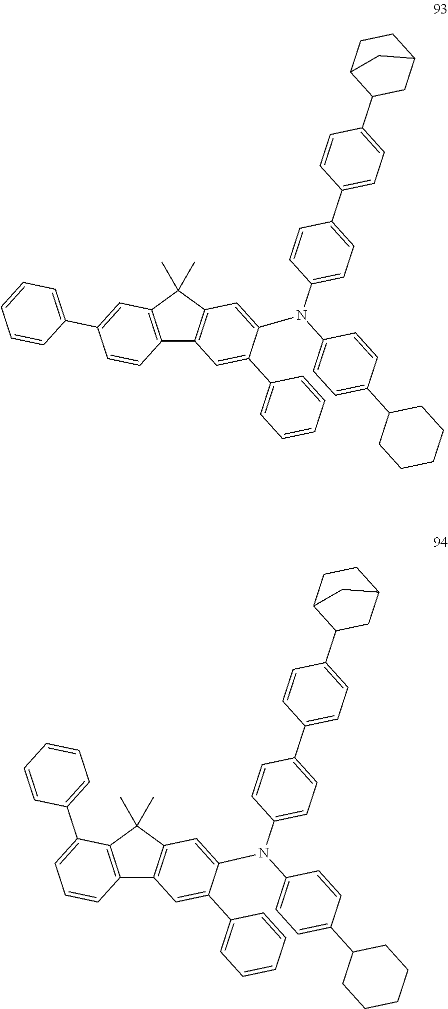 [SMILES: CC1(C)c2cc(-c3ccccc3)ccc2-c2cc(-c3ccccc3)c(N(c3ccc(-c4ccc(C5CC6CCC5C6)cc4)cc3)c3ccc(C4CCCCC4)cc3)cc21 ; CC1(C)c2cc(N(c3ccc(-c4ccc(C5CC6CCC5C6)cc4)cc3)c3ccc(C4CCCCC4)cc3)c(-c3ccccc3)cc2-c2cccc(-c3ccccc3)c21] 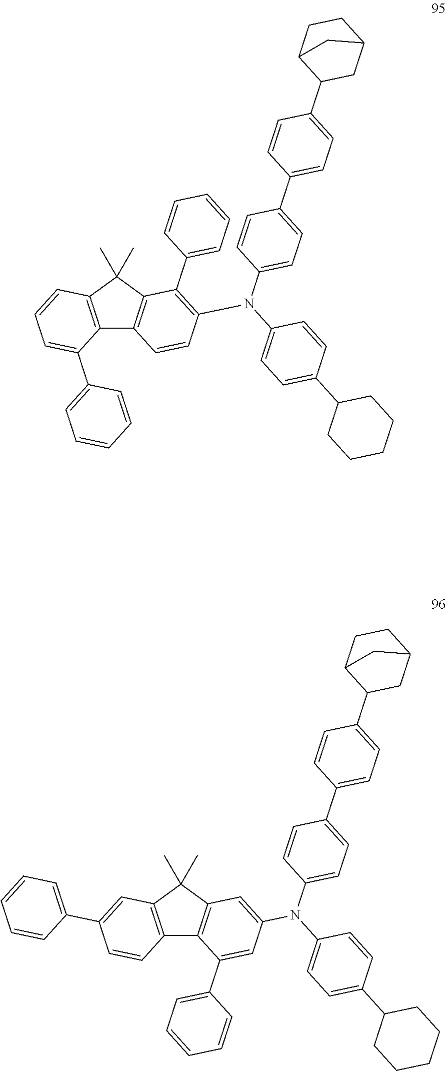 [SMILES: CC1(C)c2cccc(-c3ccccc3)c2-c2ccc(N(c3ccc(-c4ccc(C5CC6CCC5C6)cc4)cc3)c3ccc(C4CCCCC4)cc3)c(-c3ccccc3)c21 ; CC1(C)c2cc(-c3ccccc3)ccc2-c2c(-c3ccccc3)cc(N(c3ccc(-c4ccc(C5CC6CCC5C6)cc4)cc3)c3ccc(C4CCCCC4)cc3)cc21] 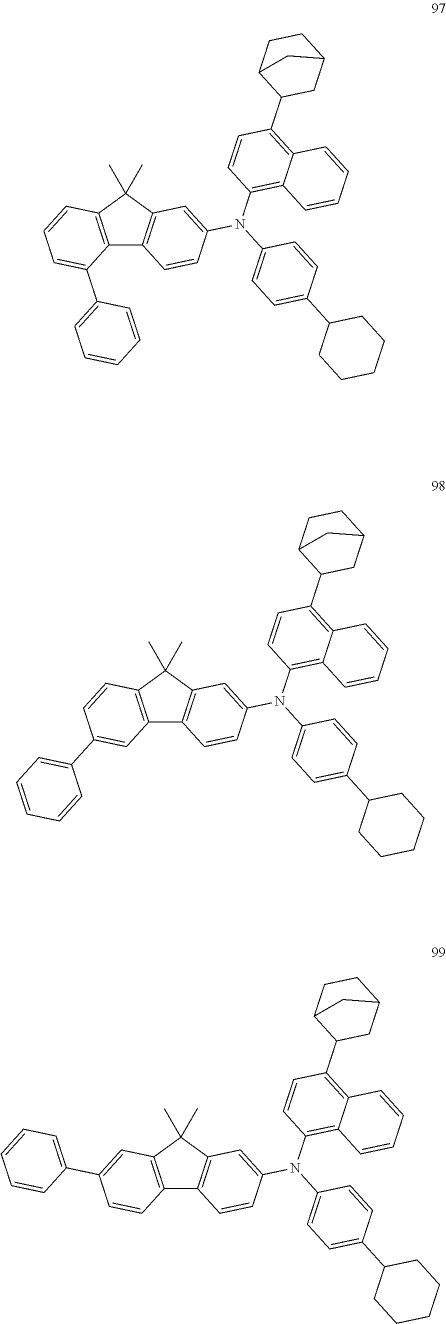 [SMILES: CC1(C)c2cc(N(c3ccc(C4CCCCC4)cc3)c3ccc(C4CC5CCC4C5)c4ccccc34)ccc2-c2c(-c3ccccc3)cccc21 ; CC1(C)c2ccc(-c3ccccc3)cc2-c2ccc(N(c3ccc(C4CCCCC4)cc3)c3ccc(C4CC5CCC4C5)c4ccccc34)cc21 ; CC1(C)c2cc(-c3ccccc3)ccc2-c2ccc(N(c3ccc(C4CCCCC4)cc3)c3ccc(C4CC5CCC4C5)c4ccccc34)cc21] 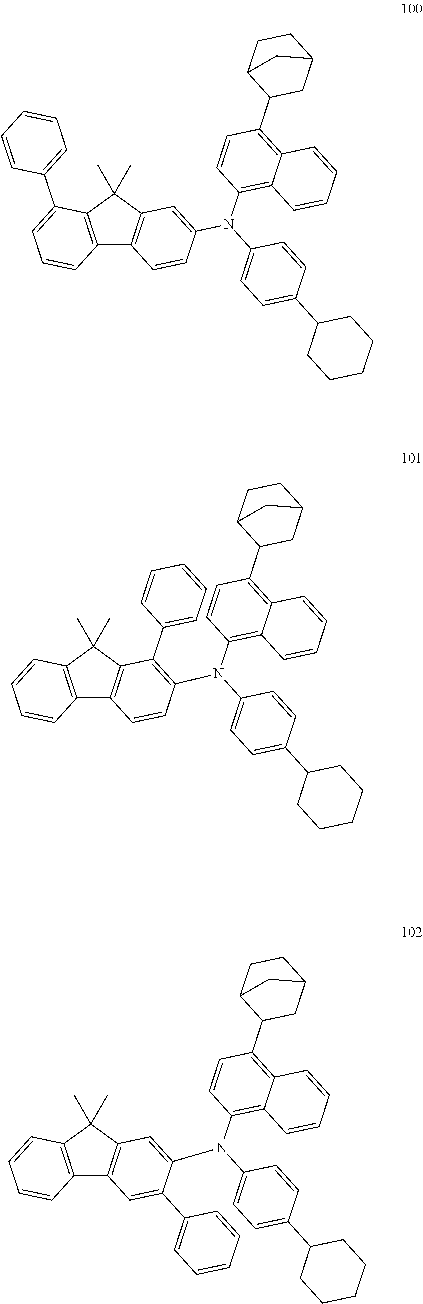 [SMILES: CC1(C)c2cc(N(c3ccc(C4CCCCC4)cc3)c3ccc(C4CC5CCC4C5)c4ccccc34)ccc2-c2cccc(-c3ccccc3)c21 ; CC1(C)c2ccccc2-c2ccc(N(c3ccc(C4CCCCC4)cc3)c3ccc(C4CC5CCC4C5)c4ccccc34)c(-c3ccccc3)c21 ; CC1(C)c2ccccc2-c2cc(-c3ccccc3)c(N(c3ccc(C4CCCCC4)cc3)c3ccc(C4CC5CCC4C5)c4ccccc34)cc21] 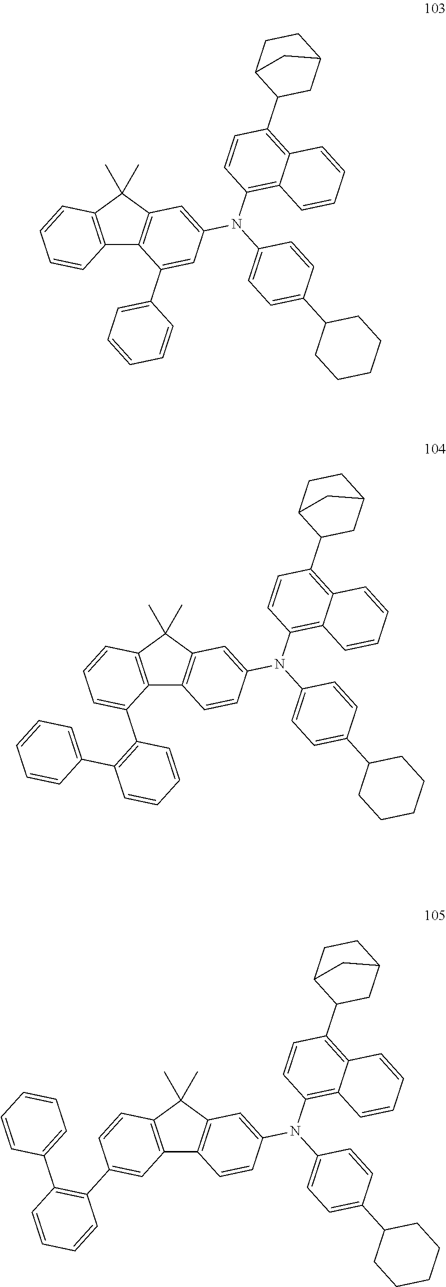 [SMILES: CC1(C)c2ccccc2-c2c(-c3ccccc3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4CC5CCC4C5)c4ccccc34)cc21] 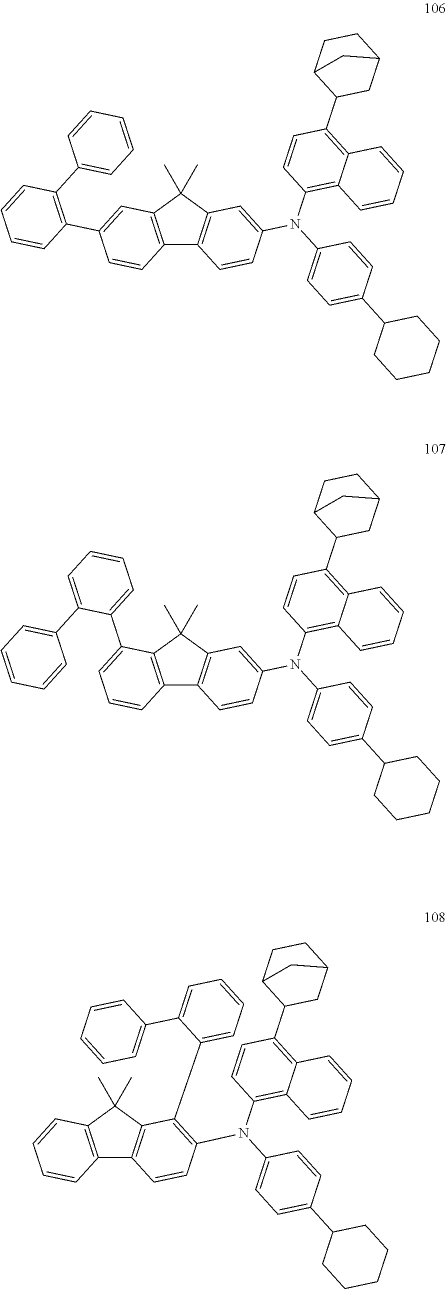 [SMILES: CC1(C)c2cc(-c3ccccc3-c3ccccc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4C5)c4ccccc34)c(-c3ccccc3-c3ccccc3)c21] 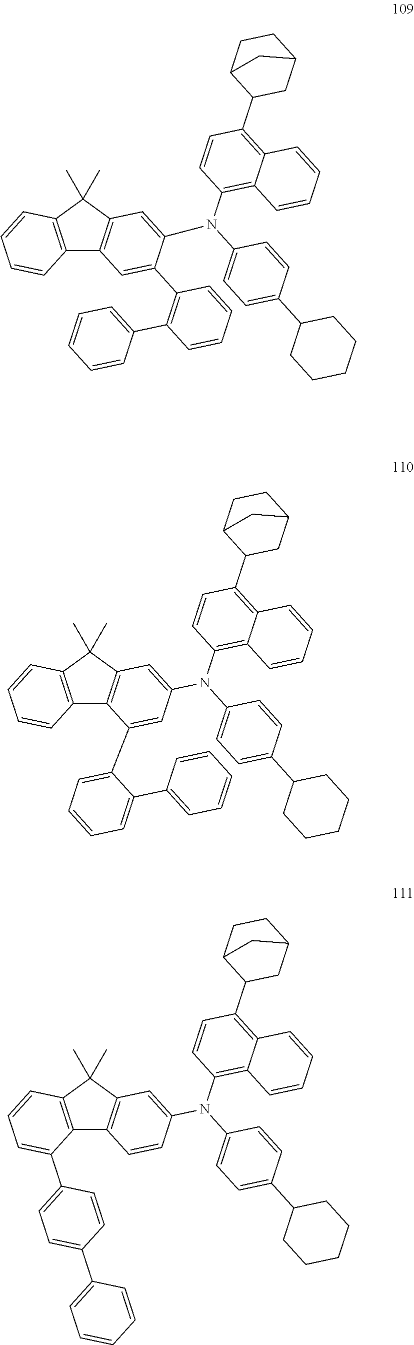 [SMILES: CC1(C)c2ccccc2-c2cc(-c3ccccc3-c3ccccc3)c(N(c3ccc(C4CCCCC4)cc3)c3ccc(C4CC5CCC4C5)c4ccccc34)cc21 ; CC1(C)c2ccccc2-c2c(-c3ccccc3-c3ccccc3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc(-c4ccccc4)cc3)cccc21] 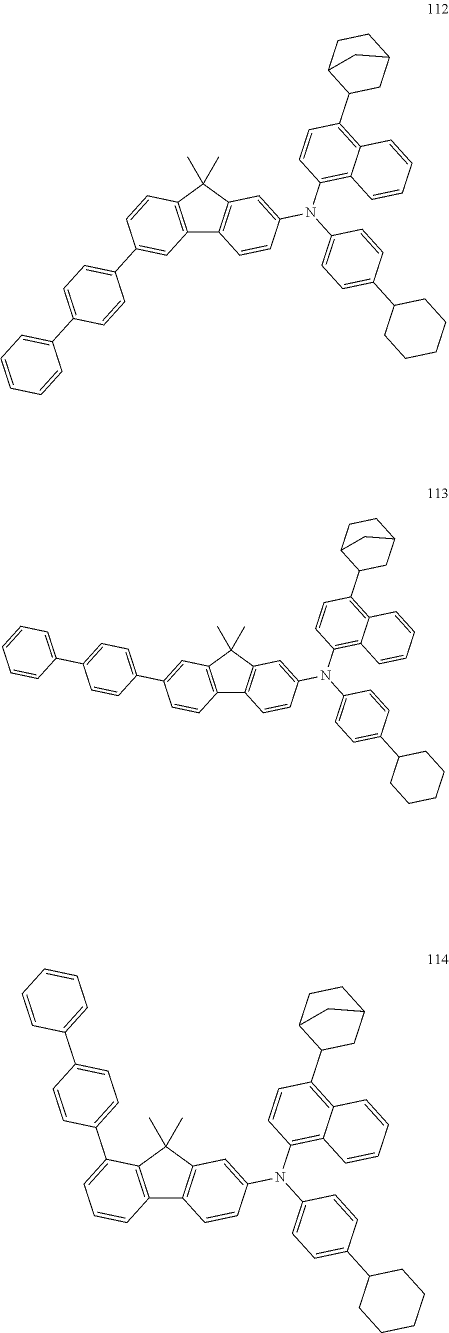 [SMILES: CC1(C)c2ccc(-c3ccc(-c4ccccc4)cc3)cc2-c2ccc(N(c3ccc(C4CCCCC4)cc3)c3ccc(C4CC5CCC4C5)c4ccccc34)cc21 ; CC1(C)c2cc(-c3ccc(-c4ccccc4)cc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc(-c4ccccc4)cc3)c21] 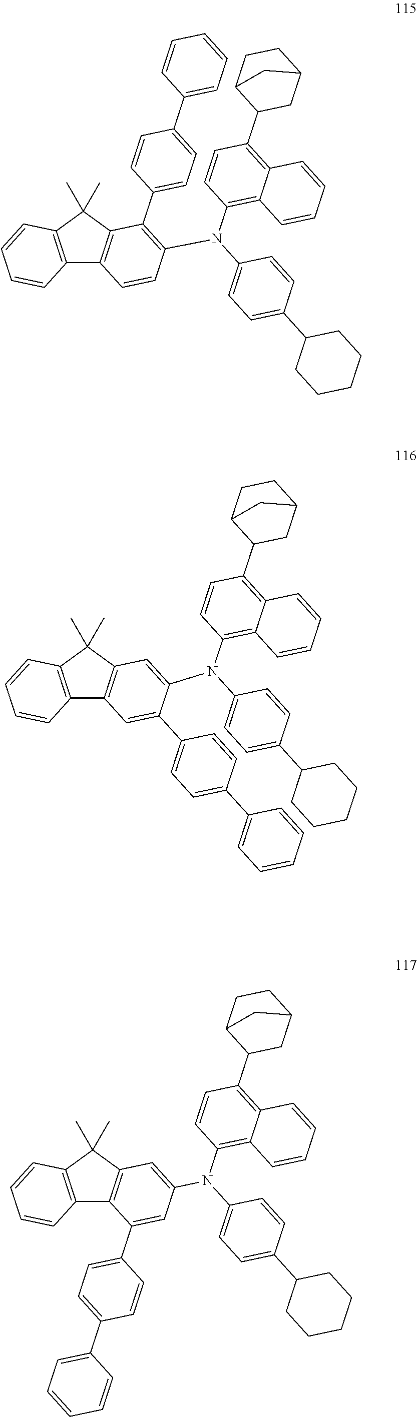 [SMILES: CC1(C)c2ccccc2-c2ccc(N(c3ccc(C4CCCCC4)cc3)c3ccc(C4CC5CCC4C5)c4ccccc34)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4CC5CCC4C5)c4ccccc34)cc21 ; CC1(C)c2ccccc2-c2c(-c3ccc(-c4ccccc4)cc3)cc(N(c3ccc(C4CCCCC4)cc3)c3ccc(C4CC5CCC4C5)c4ccccc34)cc21] 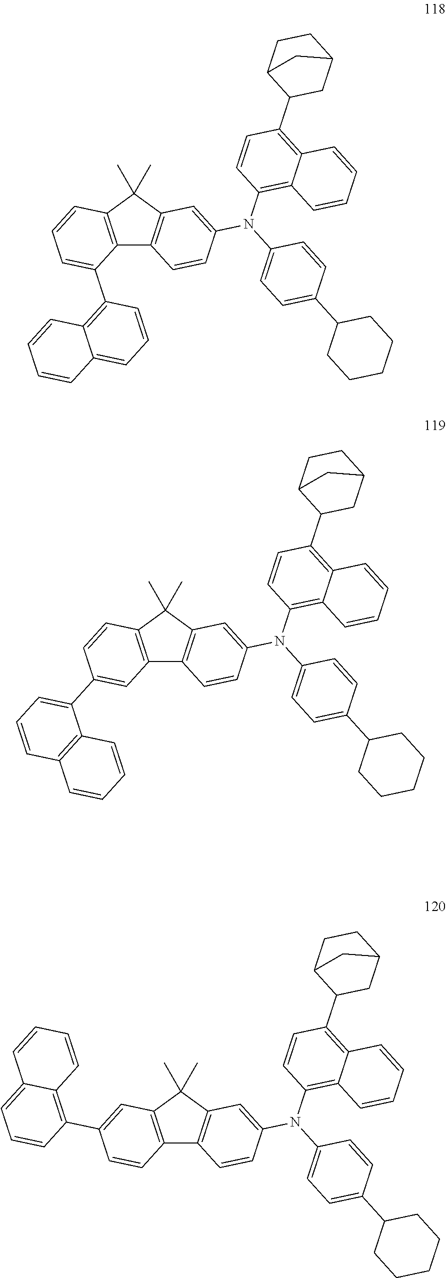 [SMILES: CC1(C)c2cc(N(c3ccc(C4CCCCC4)cc3)c3ccc(C4CC5CCC4C5)c4ccccc34)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CC5CCC4C5)c4ccccc34)cc21 ; CC1(C)c2cc(-c3cccc4ccccc34)ccc2-c2ccc(N(c3ccc(C4CCCCC4)cc3)c3ccc(C4CC5CCC4C5)c4ccccc34)cc21] 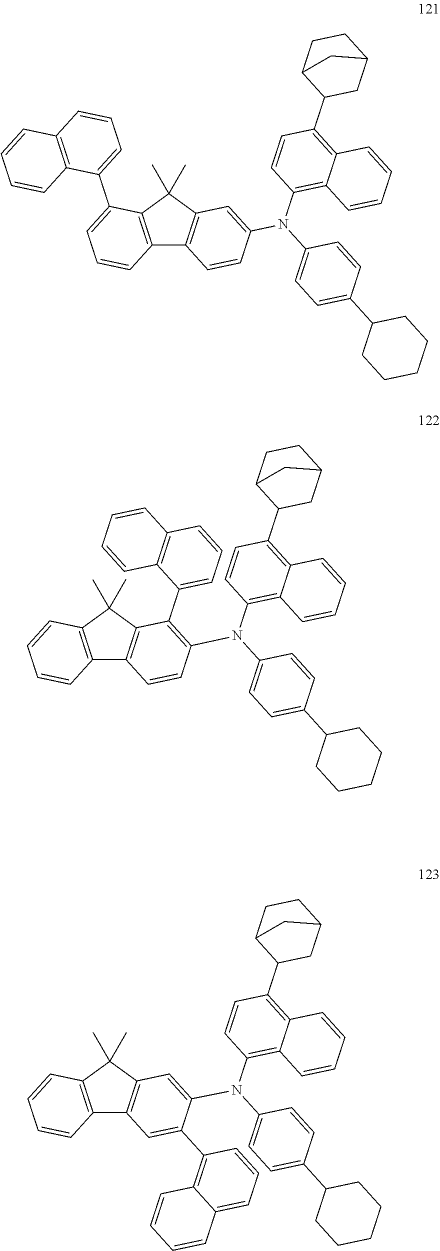 [SMILES: CC1(C)c2cc(N(c3ccc(C4CCCCC4)cc3)c3ccc(C4CC5CCC4C5)c4ccccc34)ccc2-c2cccc(-c3cccc4ccccc34)c21 ; CC1(C)c2ccccc2-c2ccc(N(c3ccc(C4CCCCC4)cc3)c3ccc(C4CC5CCC4C5)c4ccccc34)c(-c3cccc4ccccc34)c21 ; CC1(C)c2ccccc2-c2cc(-c3cccc4ccccc34)c(N(c3ccc(C4CCCCC4)cc3)c3ccc(C4CC5CCC4C5)c4ccccc34)cc21] 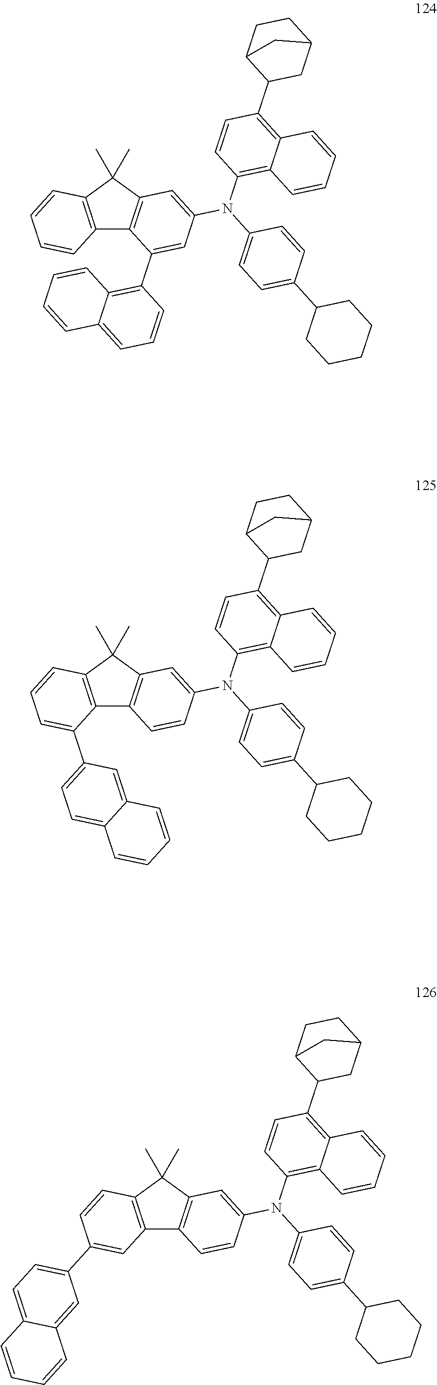 [SMILES: CC1(C)c2ccccc2-c2c(-c3cccc4ccccc34)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CC5CCC4C5)c4ccccc34)cc21] 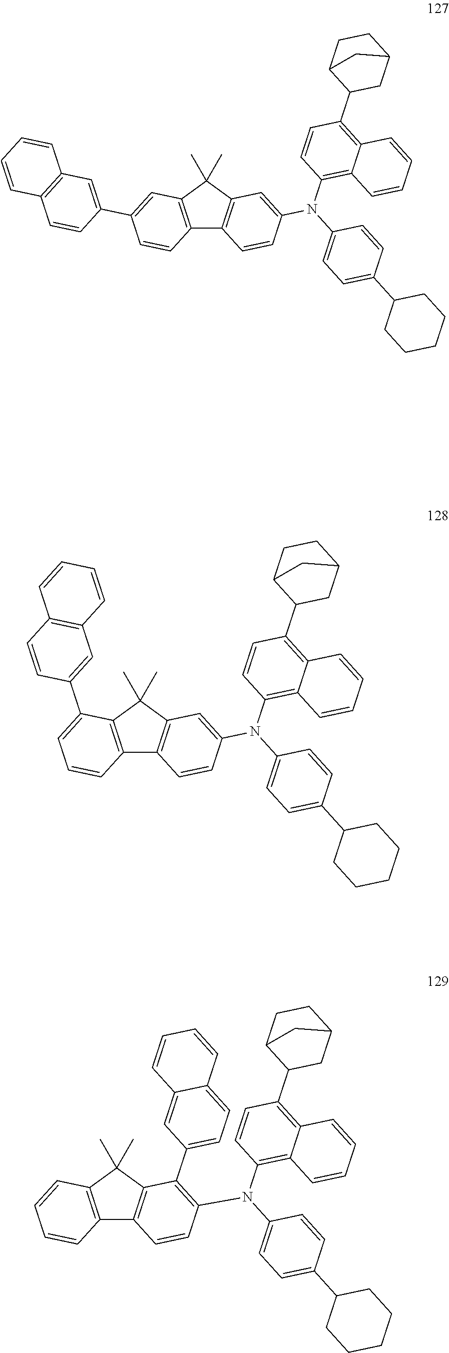 [SMILES: CC1(C)c2cc(-c3ccc4ccccc4c3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc4ccccc4c3)c21 ; CC1(C)c2ccccc2-c2ccc(N(c3ccc(C4CCCCC4)cc3)c3ccc(C4CC5CCC4C5)c4ccccc34)c(-c3ccc4ccccc4c3)c21] 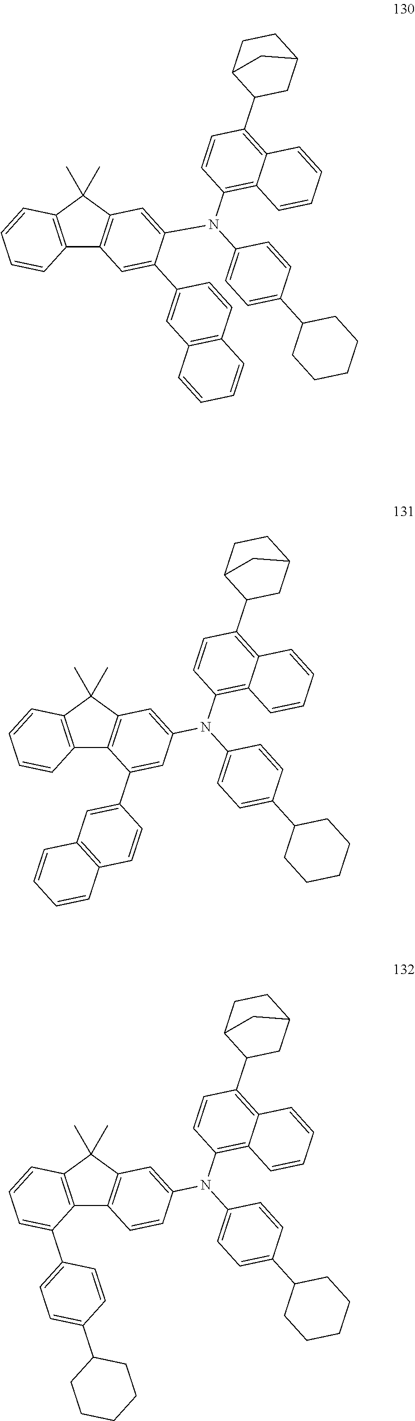 [SMILES: CC1(C)c2ccccc2-c2cc(-c3ccc4ccccc4c3)c(N(c3ccc(C4CCCCC4)cc3)c3ccc(C4CC5CCC4C5)c4ccccc34)cc21 ; CC1(C)c2ccccc2-c2c(-c3ccc4ccccc4c3)cc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2c(-c3ccc(C4CCCCC4)cc3)cccc21] 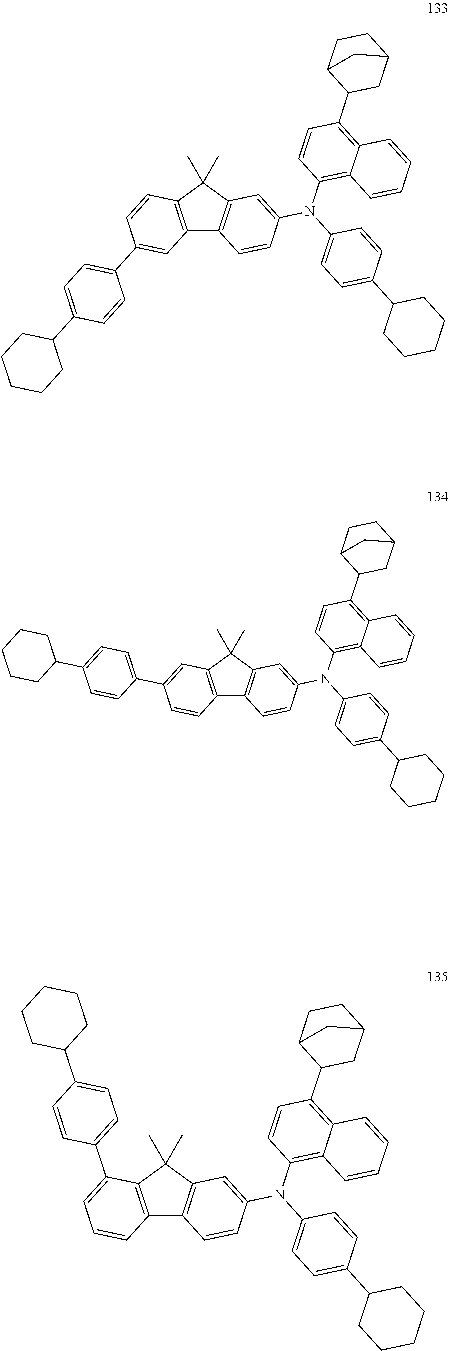 [SMILES: CC1(C)c2ccc(-c3ccc(C4CCCCC4)cc3)cc2-c2ccc(N(c3ccc(C4CCCCC4)cc3)c3ccc(C4CC5CCC4C5)c4ccccc34)cc21 ; CC1(C)c2cc(-c3ccc(C4CCCCC4)cc3)ccc2-c2ccc(N(c3ccc(C4CCCCC4)cc3)c3ccc(C4CC5CCC4C5)c4ccccc34)cc21 ; CC1(C)c2cc(N(c3ccc(C4CCCCC4)cc3)c3ccc(C4CC5CCC4C5)c4ccccc34)ccc2-c2cccc(-c3ccc(C4CCCCC4)cc3)c21] 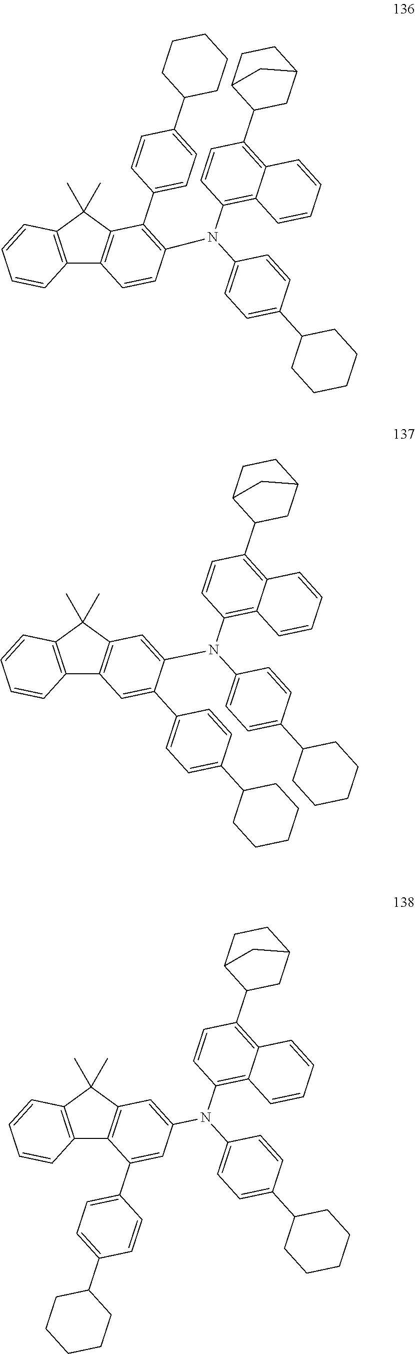 [SMILES: CC1(C)c2ccccc2-c2ccc(N(c3ccc(C4CCCCC4)cc3)c3ccc(C4CC5CCC4C5)c4ccccc34)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C4CC5CCC4C5)c4ccccc34)cc21 ; CC1(C)c2ccccc2-c2c(-c3ccc(C4CCCCC4)cc3)cc(N(c3ccc(C4CCCCC4)cc3)c3ccc(C4CC5CCC4C5)c4ccccc34)cc21] 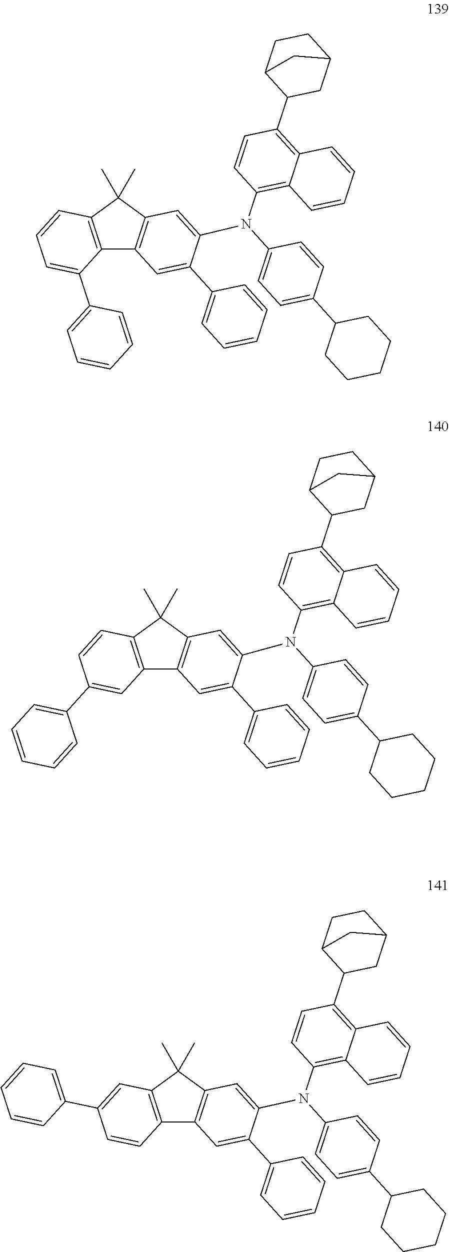 [SMILES: CC1(C)c2cc(N(c3ccc(C4CCCCC4)cc3)c3ccc(C4CC5CCC4C5)c4ccccc34)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4CC5CCC4C5)c4ccccc34)cc21 ; CC1(C)c2cc(-c3ccccc3)ccc2-c2cc(-c3ccccc3)c(N(c3ccc(C4CCCCC4)cc3)c3ccc(C4CC5CCC4C5)c4ccccc34)cc21] 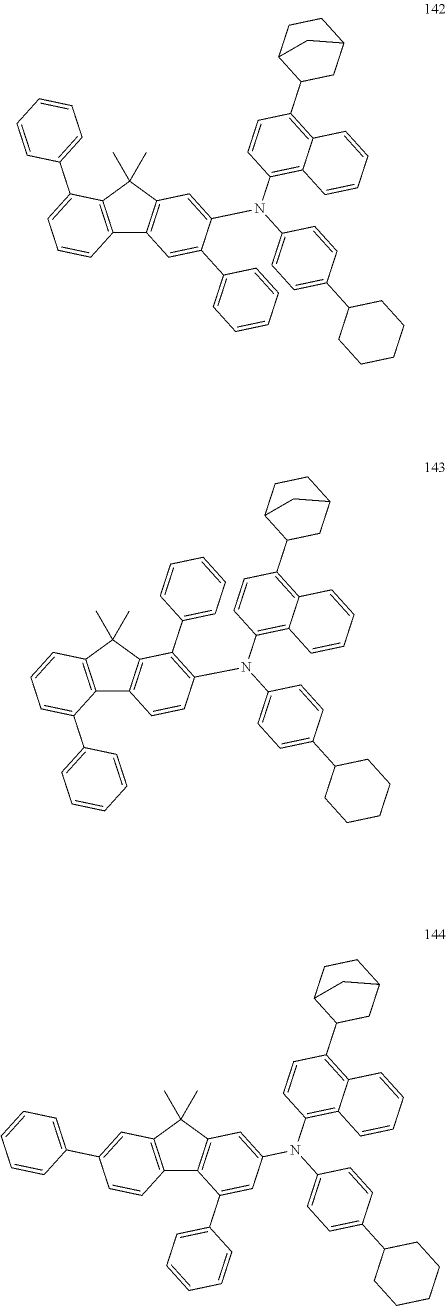 [SMILES: CC1(C)c2cc(N(c3ccc(C4CCCCC4)cc3)c3ccc(C4CC5CCC4C5)c4ccccc34)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4CC5CCC4C5)c4ccccc34)c(-c3ccccc3)c21 ; CC1(C)c2cc(-c3ccccc3)ccc2-c2c(-c3ccccc3)cc(N(c3ccc(C4CCCCC4)cc3)c3ccc(C4CC5CCC4C5)c4ccccc34)cc21] 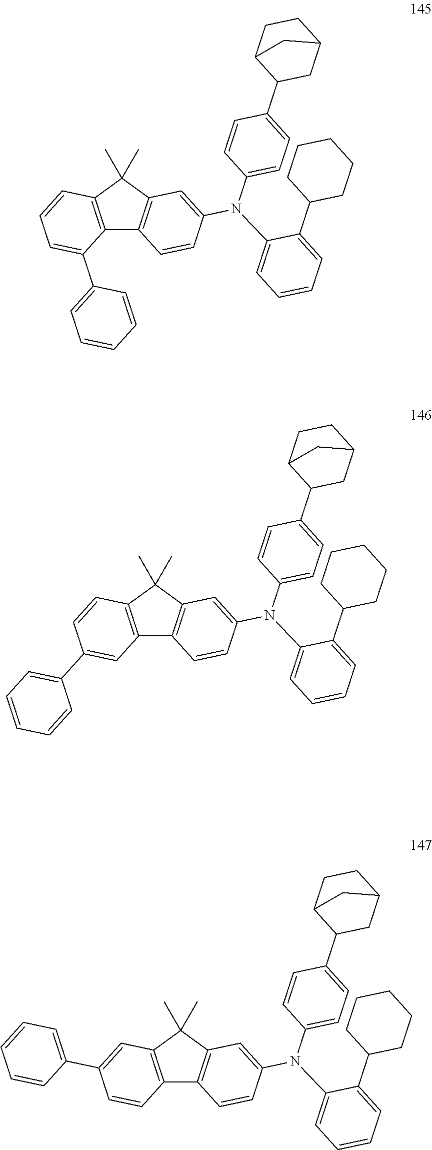 [SMILES: CC1(C)c2cc(N(c3ccc(C4CC5CCC4C5)cc3)c3ccccc3C3CCCCC3)ccc2-c2c(-c3ccccc3)cccc21 ; CC1(C)c2ccc(-c3ccccc3)cc2-c2ccc(N(c3ccc(C4CC5CCC4C5)cc3)c3ccccc3C3CCCCC3)cc21 ; CC1(C)c2cc(-c3ccccc3)ccc2-c2ccc(N(c3ccc(C4CC5CCC4C5)cc3)c3ccccc3C3CCCCC3)cc21] 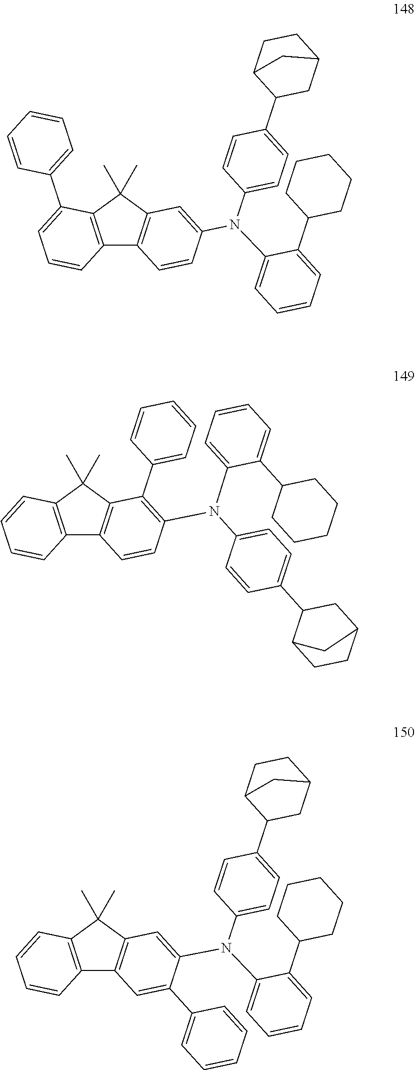 [SMILES: CC1(C)c2cc(N(c3ccc(C4CC5CCC4C5)cc3)c3ccccc3C3CCCCC3)ccc2-c2cccc(-c3ccccc3)c21 ; CC1(C)c2ccccc2-c2ccc(N(c3ccc(C4CC5CCC4C5)cc3)c3ccccc3C3CCCCC3)c(-c3ccccc3)c21 ; CC1(C)c2ccccc2-c2cc(-c3ccccc3)c(N(c3ccc(C4CC5CCC4C5)cc3)c3ccccc3C3CCCCC3)cc21] 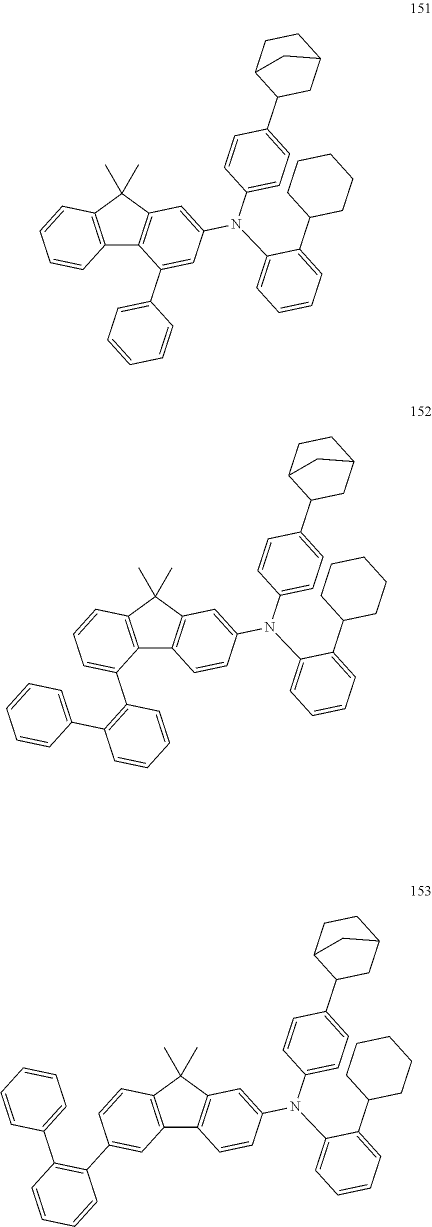 [SMILES: CC1(C)c2ccccc2-c2c(-c3ccccc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3ccccc3C3CCCCC3)cc21] 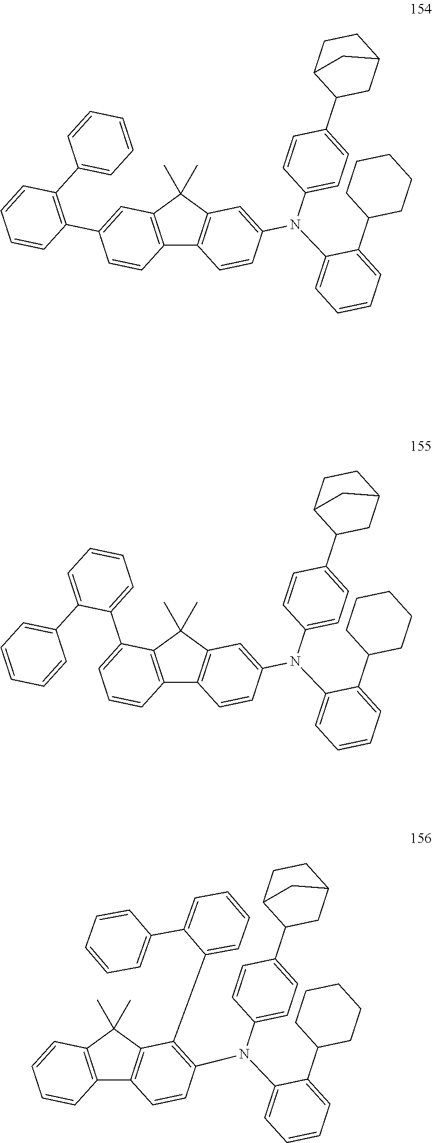 [SMILES: CC1(C)c2cc(-c3ccccc3-c3ccccc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccccc3-c3ccccc3)c21 ; CC1(C)c2ccccc2-c2ccc(N(c3ccc(C4CC5CCC4C5)cc3)c3ccccc3C3CCCCC3)c(-c3ccccc3-c3ccccc3)c21] 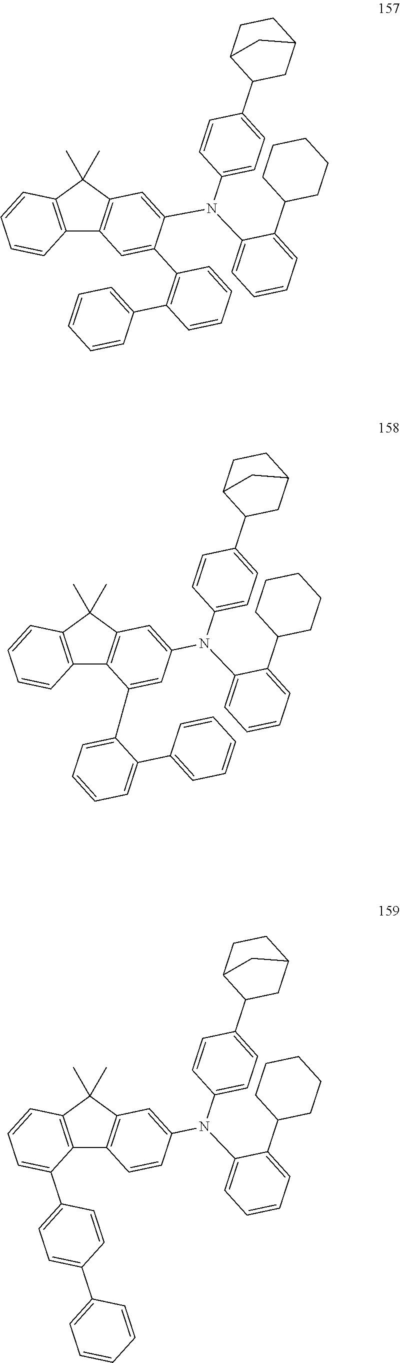 [SMILES: CC1(C)c2ccccc2-c2cc(-c3ccccc3-c3ccccc3)c(N(c3ccc(C4CC5CCC4C5)cc3)c3ccccc3C3CCCCC3)cc21 ; CC1(C)c2ccccc2-c2c(-c3ccccc3-c3ccccc3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc(-c4ccccc4)cc3)cccc21] 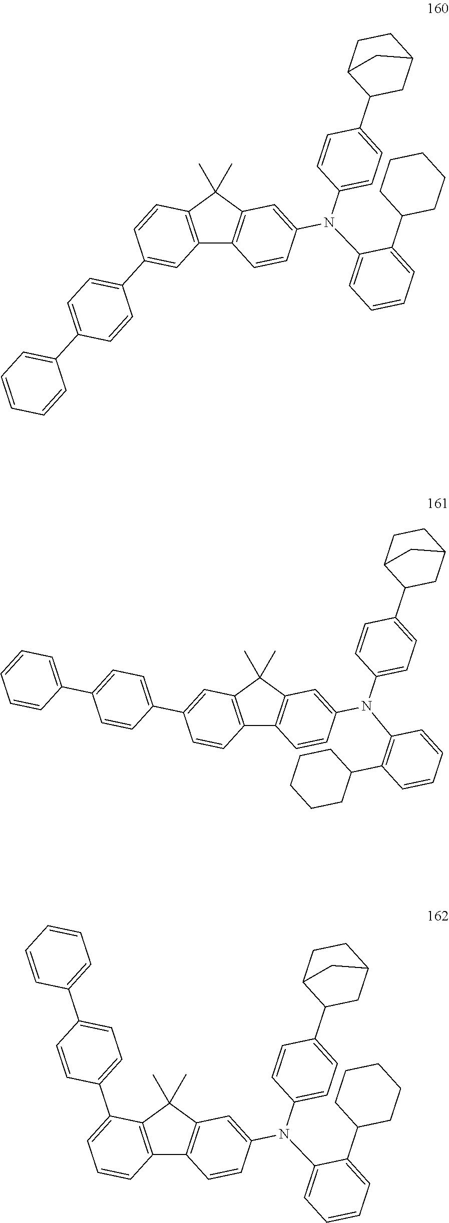 [SMILES: CC1(C)c2ccc(-c3ccc(-c4ccccc4)cc3)cc2-c2ccc(N(c3ccc(C4CC5CCC4C5)cc3)c3ccccc3C3CCCCC3)cc21 ; CC1(C)c2cc(-c3ccc(-c4ccccc4)cc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc(-c4ccccc4)cc3)c21] 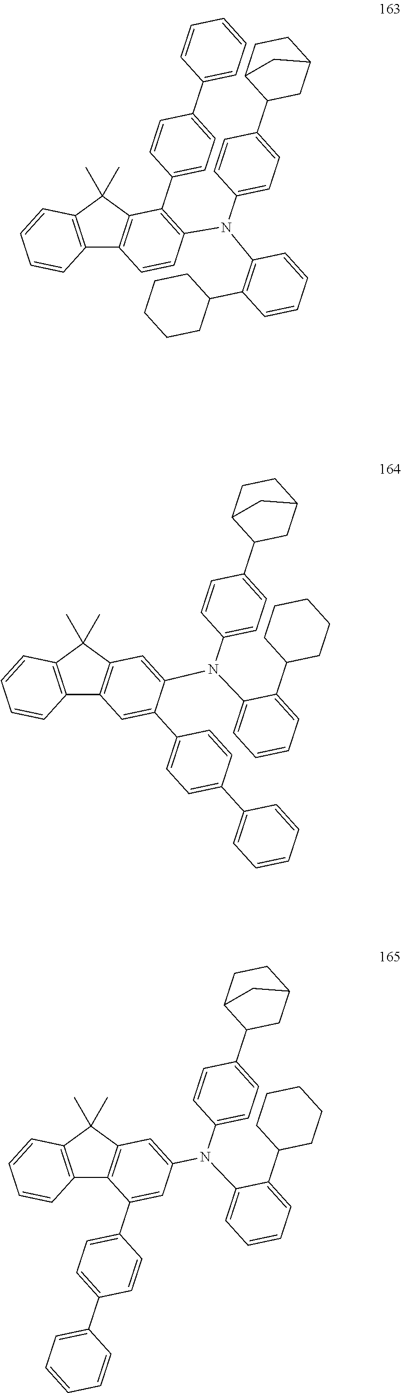 [SMILES: CC1(C)c2ccccc2-c2ccc(N(c3ccc(C4CC5CCC4C5)cc3)c3ccccc3C3CCCCC3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3ccccc3C3CCCCC3)cc21 ; CC1(C)c2ccccc2-c2c(-c3ccc(-c4ccccc4)cc3)cc(N(c3ccc(C4CC5CCC4C5)cc3)c3ccccc3C3CCCCC3)cc21] 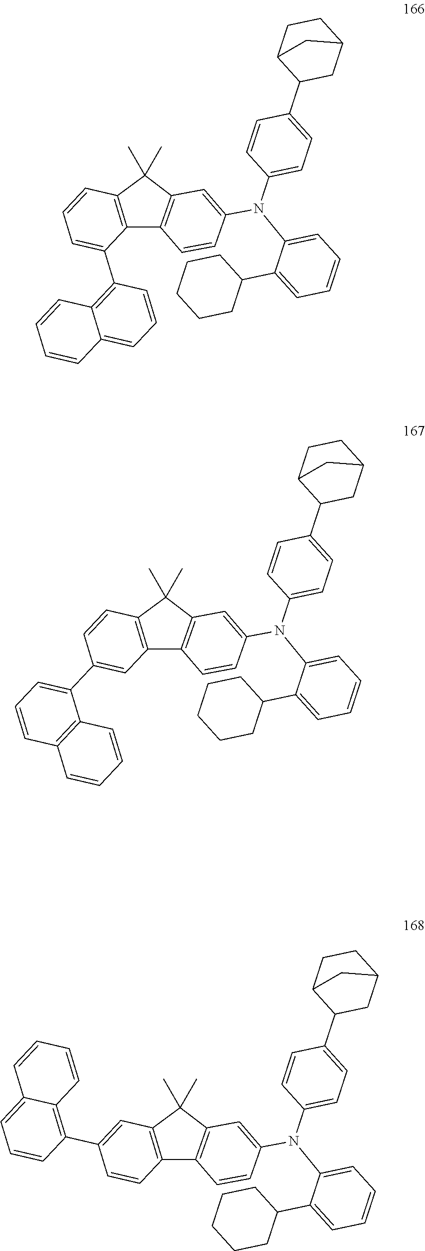 [SMILES: CC1(C)c2cc(N(c3ccc(C4CC5CCC4C5)cc3)c3ccccc3C3CCCCC3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccccc3C3CCCCC3)cc21 ; CC1(C)c2cc(-c3cccc4ccccc34)ccc2-c2ccc(N(c3ccc(C4CC5CCC4C5)cc3)c3ccccc3C3CCCCC3)cc21] 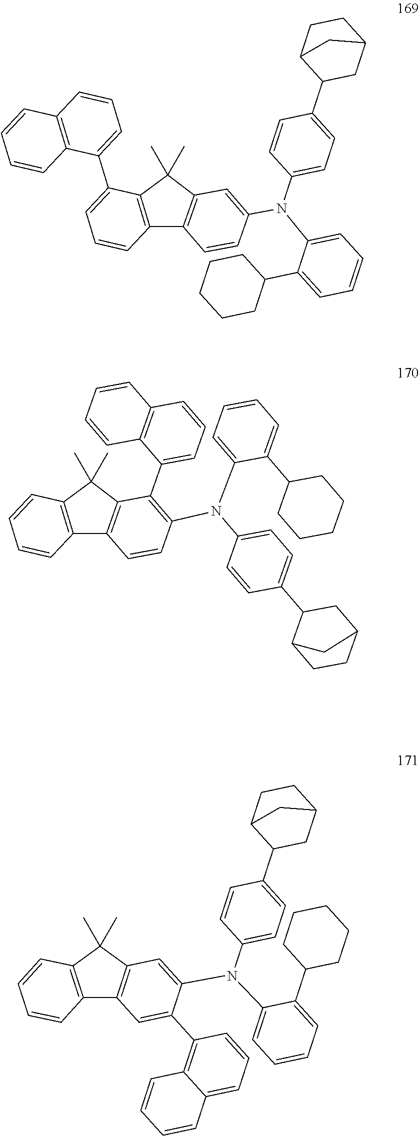 [SMILES: CC1(C)c2cc(N(c3ccc(C4CC5CCC4C5)cc3)c3ccccc3C3CCCCC3)ccc2-c2cccc(-c3cccc4ccccc34)c21 ; CC1(C)c2ccccc2-c2ccc(N(c3ccc(C4CC5CCC4C5)cc3)c3ccccc3C3CCCCC3)c(-c3cccc4ccccc34)c21 ; CC1(C)c2ccccc2-c2cc(-c3cccc4ccccc34)c(N(c3ccc(C4CC5CCC4C5)cc3)c3ccccc3C3CCCCC3)cc21] 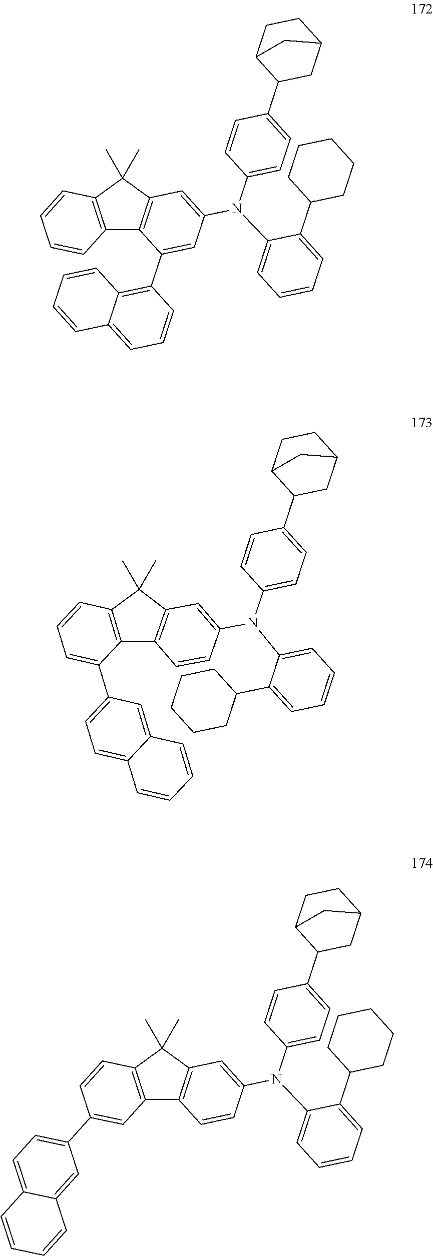 [SMILES: CC1(C)c2ccccc2-c2c(-c3cccc4ccccc34)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccccc3C3CCCCC3)cc21] 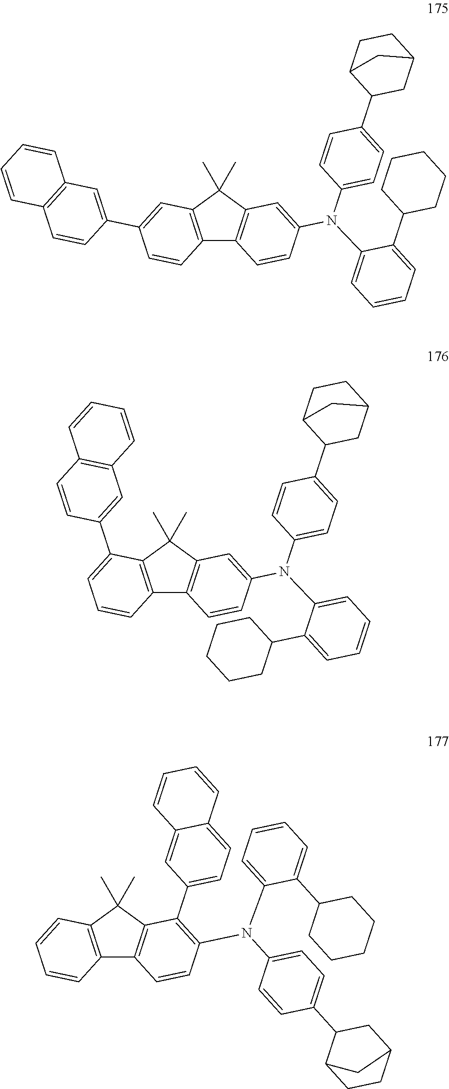 [SMILES: CC1(C)c2cc(-c3ccc4ccccc4c3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc4ccccc4c3)c21 ; CC1(C)c2ccccc2-c2ccc(N(c3ccc(C4CC5CCC4C5)cc3)c3ccccc3C3CCCCC3)c(-c3ccc4ccccc4c3)c21] 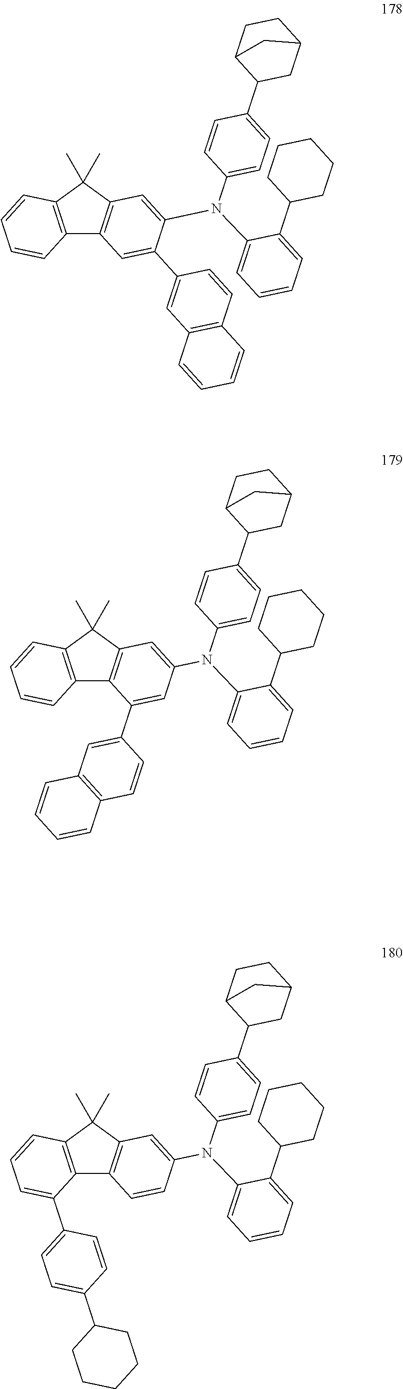 [SMILES: CC1(C)c2ccccc2-c2cc(-c3ccc4ccccc4c3)c(N(c3ccc(C4CC5CCC4C5)cc3)c3ccccc3C3CCCCC3)cc21 ; CC1(C)c2ccccc2-c2c(-c3ccc4ccccc4c3)cc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2c(-c3ccc(C4CCCCC4)cc3)cccc21] 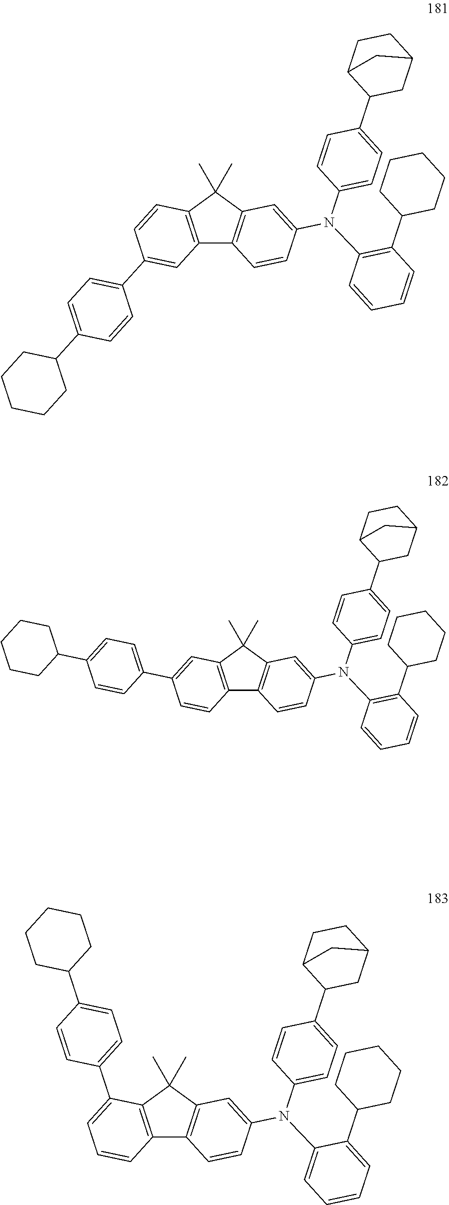 [SMILES: CC1(C)c2ccc(-c3ccc(C4CCCCC4)cc3)cc2-c2ccc(N(c3ccc(C4CC5CCC4C5)cc3)c3ccccc3C3CCCCC3)cc21 ; CC1(C)c2cc(-c3ccc(C4CCCCC4)cc3)ccc2-c2ccc(N(c3ccc(C4CC5CCC4C5)cc3)c3ccccc3C3CCCCC3)cc21 ; CC1(C)c2cc(N(c3ccc(C4CC5CCC4C5)cc3)c3ccccc3C3CCCCC3)ccc2-c2cccc(-c3ccc(C4CCCCC4)cc3)c21] 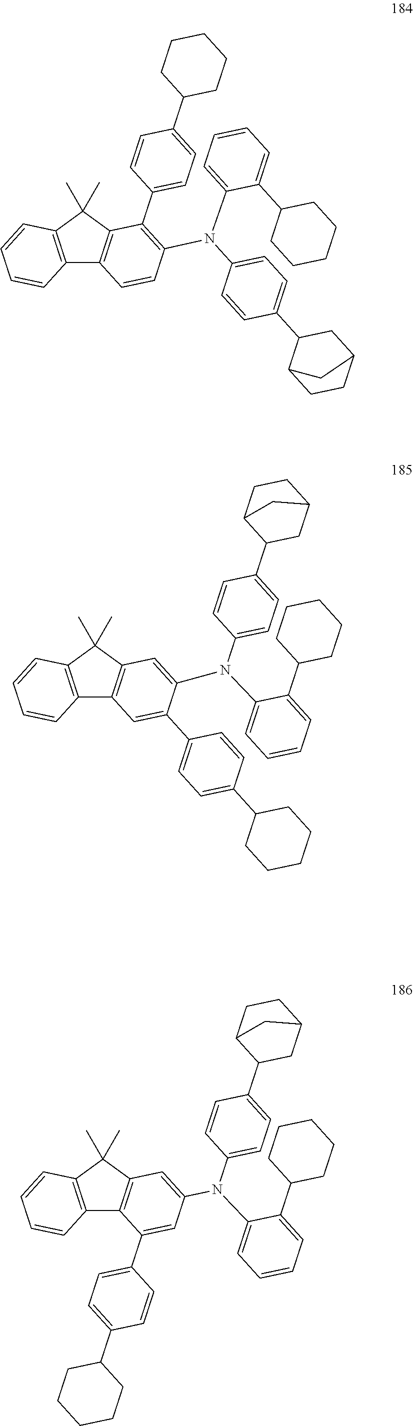 [SMILES: CC1(C)c2ccccc2-c2ccc(N(c3ccc(C4CC5CCC4C5)cc3)c3ccccc3C3CCCCC3)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3ccccc3C3CCCCC3)cc21 ; CC1(C)c2ccccc2-c2c(-c3ccc(C4CCCCC4)cc3)cc(N(c3ccc(C4CC5CCC4C5)cc3)c3ccccc3C3CCCCC3)cc21] 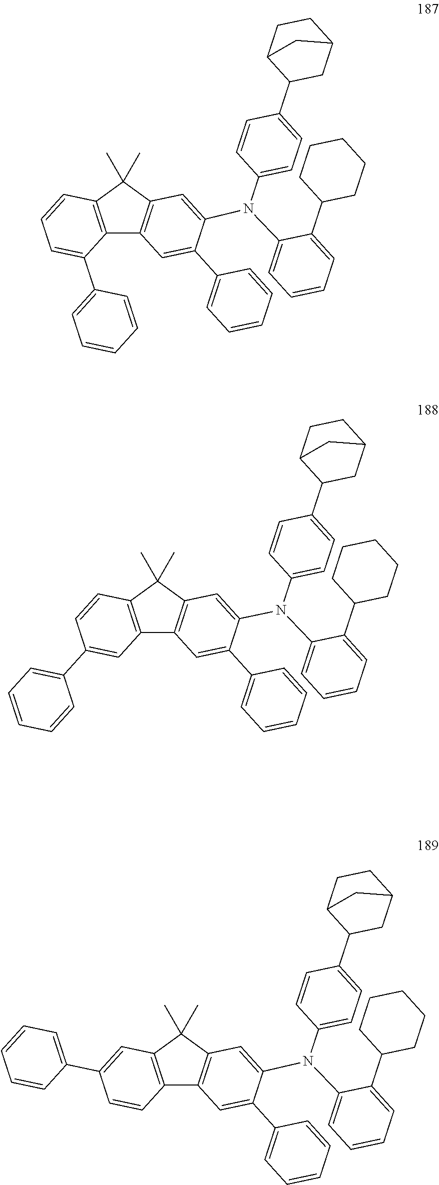 [SMILES: CC1(C)c2cc(N(c3ccc(C4CC5CCC4C5)cc3)c3ccccc3C3CCCCC3)c(-c3ccccc3)cc2-c2c(-c3ccccc3)cccc21 ; CC1(C)c2ccc(-c3ccccc3)cc2-c2cc(-c3ccccc3)c(N(c3ccc(C4CC5CCC4C5)cc3)c3ccccc3C3CCCCC3)cc21 ; CC1(C)c2cc(-c3ccccc3)ccc2-c2cc(-c3ccccc3)c(N(c3ccc(C4CC5CCC4C5)cc3)c3ccccc3C3CCCCC3)cc21] 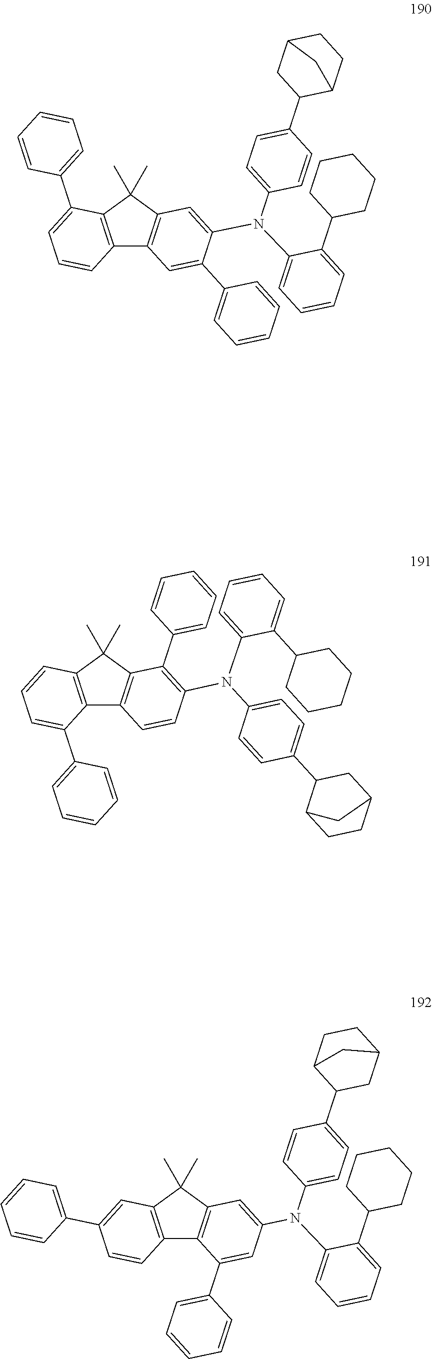 [SMILES: CC1(C)c2cc(N(c3ccc(C4CC5CCC4C5)cc3)c3ccccc3C3CCCCC3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3ccccc3C3CCCCC3)c(-c3ccccc3)c21 ; CC1(C)c2cc(-c3ccccc3)ccc2-c2c(-c3ccccc3)cc(N(c3ccc(C4CC5CCC4C5)cc3)c3ccccc3C3CCCCC3)cc21] 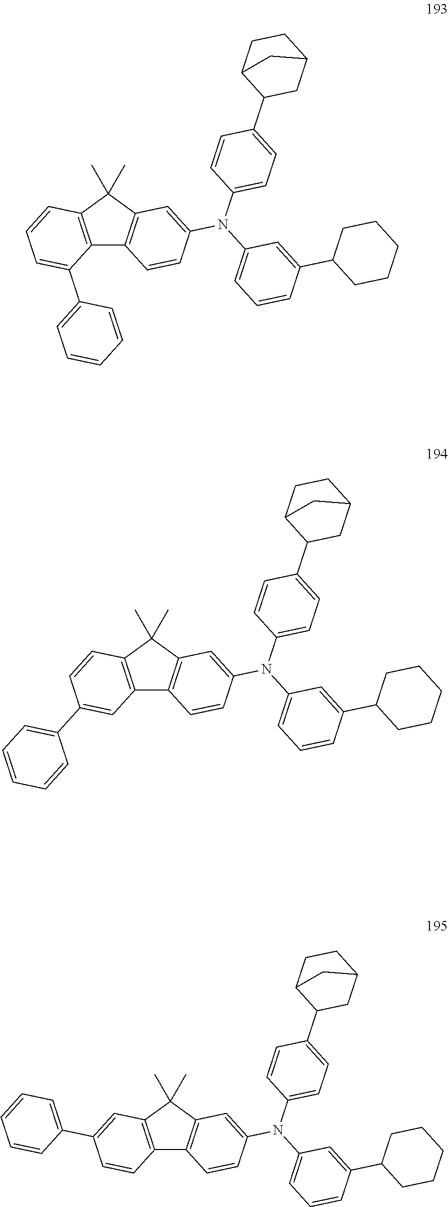 [SMILES: CC1(C)c2cc(N(c3ccc(C4CC5CCC4C5)cc3)c3cccc(C4CCCCC4)c3)ccc2-c2c(-c3ccccc3)cccc21 ; CC1(C)c2ccc(-c3ccccc3)cc2-c2ccc(N(c3ccc(C4CC5CCC4C5)cc3)c3cccc(C4CCCCC4)c3)cc21 ; CC1(C)c2cc(-c3ccccc3)ccc2-c2ccc(N(c3ccc(C4CC5CCC4C5)cc3)c3cccc(C4CCCCC4)c3)cc21] 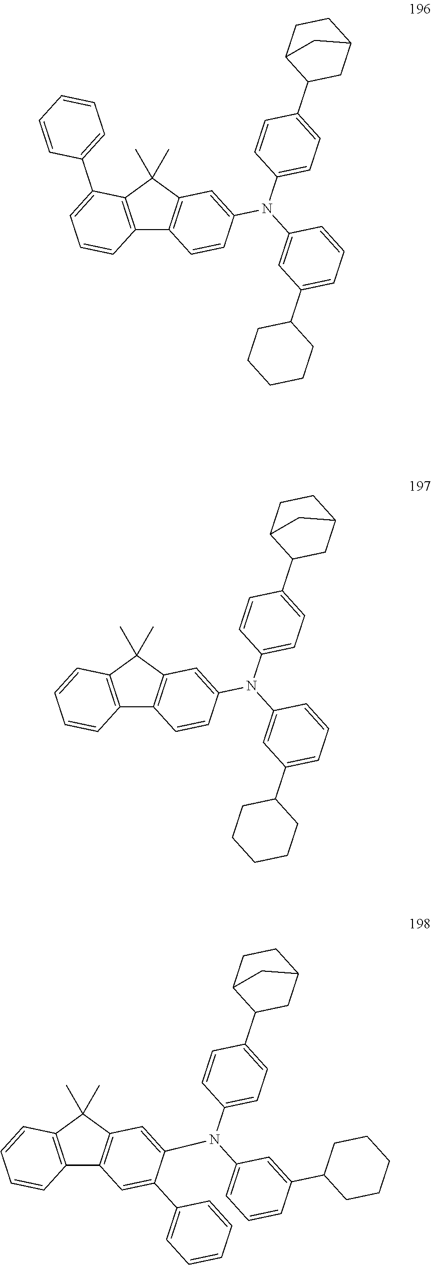 [SMILES: CC1(C)c2cc(N(c3ccc(C4CC5CCC4C5)cc3)c3cccc(C4CCCCC4)c3)ccc2-c2cccc(-c3ccccc3)c21 ; CC1(C)c2ccccc2-c2ccc(N(c3ccc(C4CC5CCC4C5)cc3)c3cccc(C4CCCCC4)c3)cc21 ; CC1(C)c2ccccc2-c2cc(-c3ccccc3)c(N(c3ccc(C4CC5CCC4C5)cc3)c3cccc(C4CCCCC4)c3)cc21] 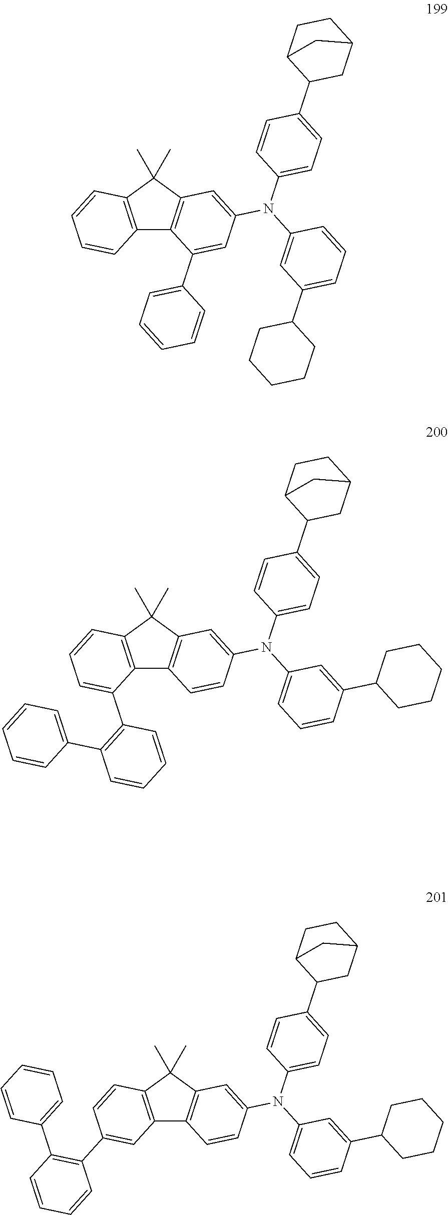 [SMILES: CC1(C)c2ccccc2-c2c(-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccccc3-c3ccccc3)cccc21 ; CC1(C)c2ccc(-c3ccccc3-c3ccccc3)cc2-c2ccc(N(c3ccc(C4CC5CCC4C5)cc3)c3cccc(C4CCCCC4)c3)cc21] 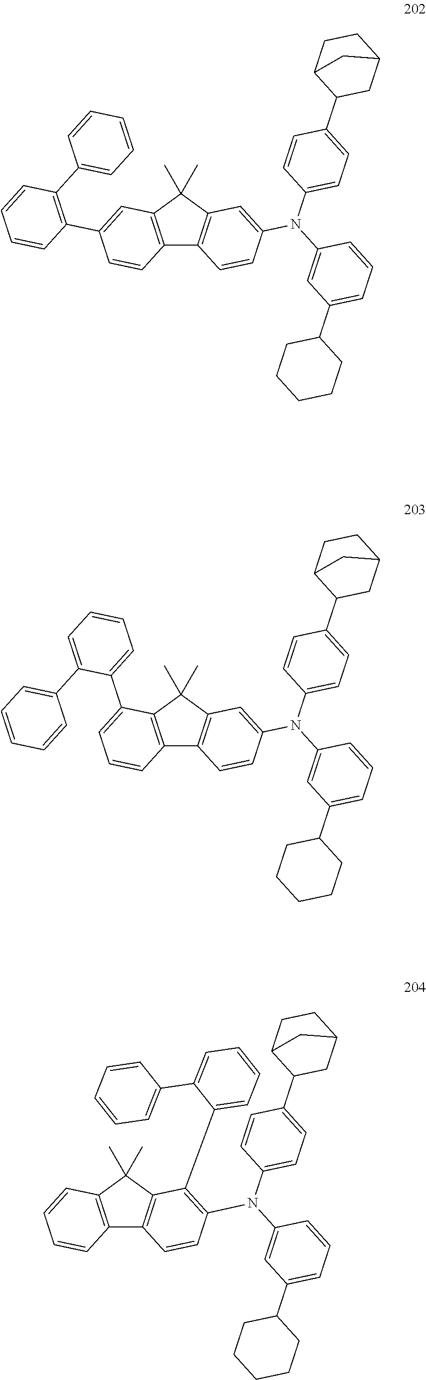 [SMILES: CC1(C)c2cc(-c3ccccc3-c3ccccc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccccc3-c3ccccc3)c21 ; CC1(C)c2ccccc2-c2ccc(N(c3ccc(C4CC5CCC4C5)cc3)c3cccc(C4CCCCC4)c3)c(-c3ccccc3-c3ccccc3)c21] 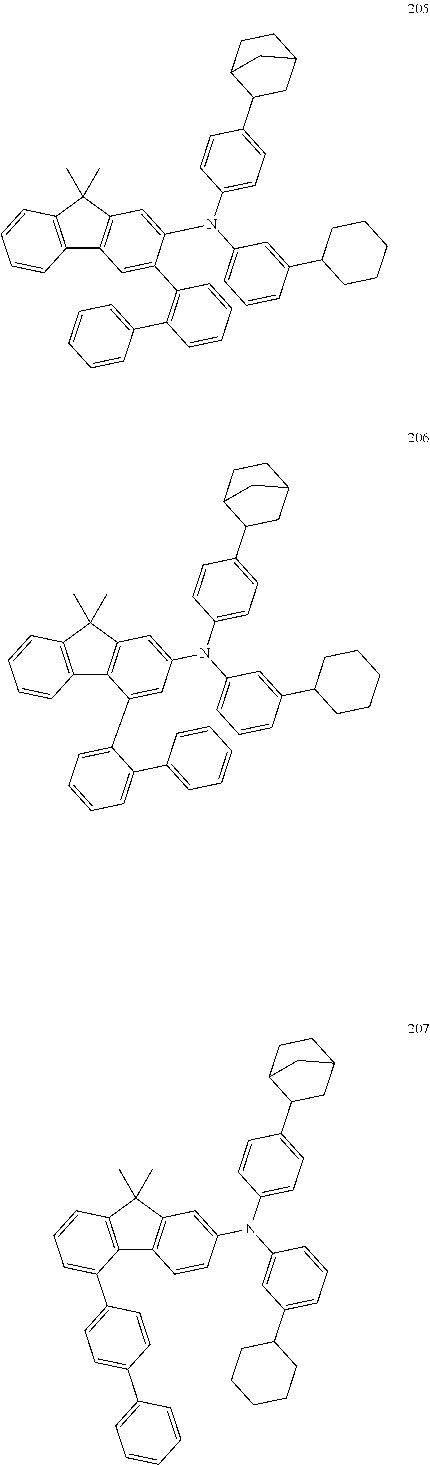 [SMILES: CC1(C)c2ccccc2-c2cc(-c3ccccc3-c3ccccc3)c(N(c3ccc(C4CC5CCC4C5)cc3)c3cccc(C4CCCCC4)c3)cc21 ; CC1(C)c2ccccc2-c2c(-c3ccccc3-c3ccccc3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc(-c4ccccc4)cc3)cccc21] 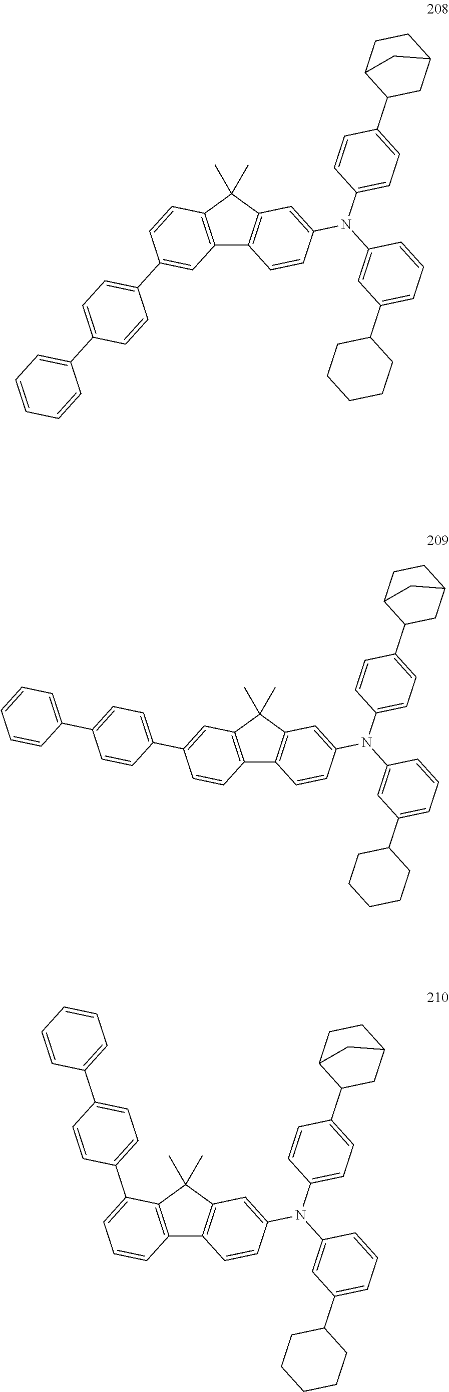 [SMILES: CC1(C)c2ccc(-c3ccc(-c4ccccc4)cc3)cc2-c2ccc(N(c3ccc(C4CC5CCC4C5)cc3)c3cccc(C4CCCCC4)c3)cc21 ; CC1(C)c2cc(-c3ccc(-c4ccccc4)cc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc(-c4ccccc4)cc3)c21] 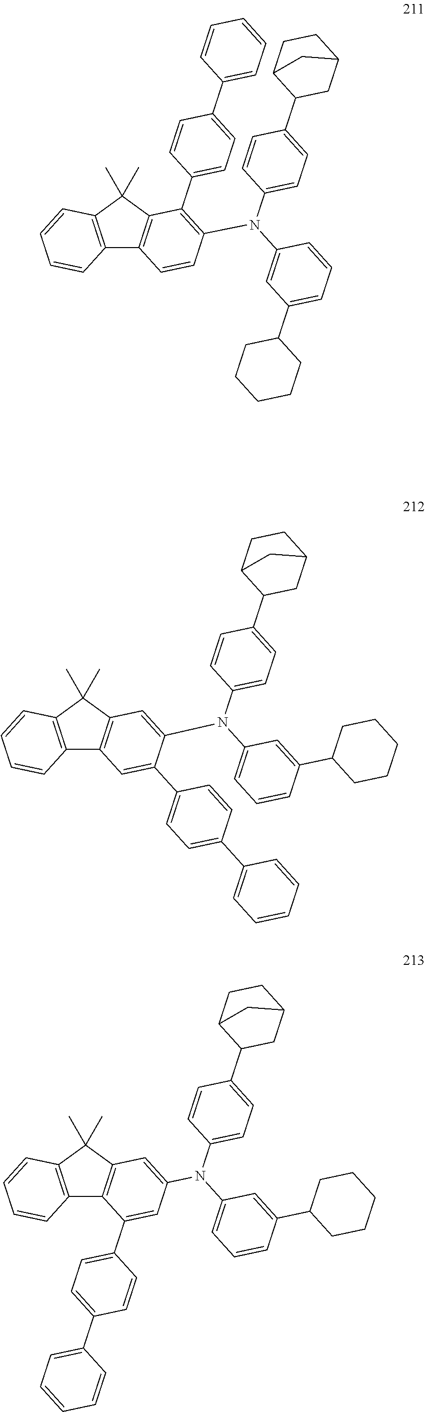 [SMILES: CC1(C)c2ccccc2-c2ccc(N(c3ccc(C4CC5CCC4C5)cc3)c3cccc(C4CCCCC4)c3)c(-c3ccc(-c4ccccc4)cc3)c21 ; CC1(C)c2ccccc2-c2cc(-c3ccc(-c4ccccc4)cc3)c(N(c3ccc(C4CC5CCC4C5)cc3)c3cccc(C4CCCCC4)c3)cc21 ; CC1(C)c2ccccc2-c2c(-c3ccc(-c4ccccc4)cc3)cc(N(c3ccc(C4CC5CCC4C5)cc3)c3cccc(C4CCCCC4)c3)cc21] 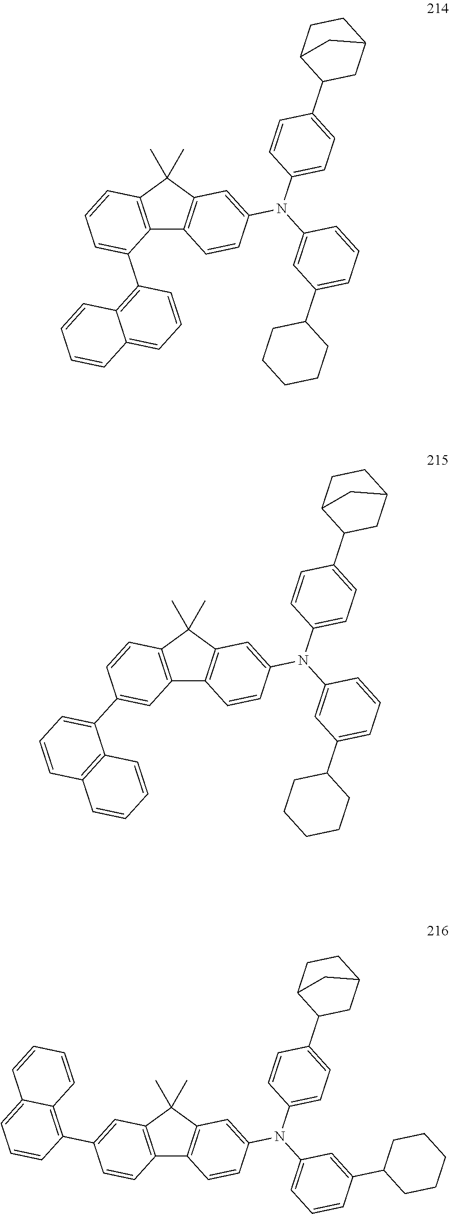 [SMILES: CC1(C)c2cc(N(c3ccc(C4CC5CCC4C5)cc3)c3cccc(C4CCCCC4)c3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3cccc(C4CCCCC4)c3)cc21 ; CC1(C)c2cc(-c3cccc4ccccc34)ccc2-c2ccc(N(c3ccc(C4CC5CCC4C5)cc3)c3cccc(C4CCCCC4)c3)cc21] 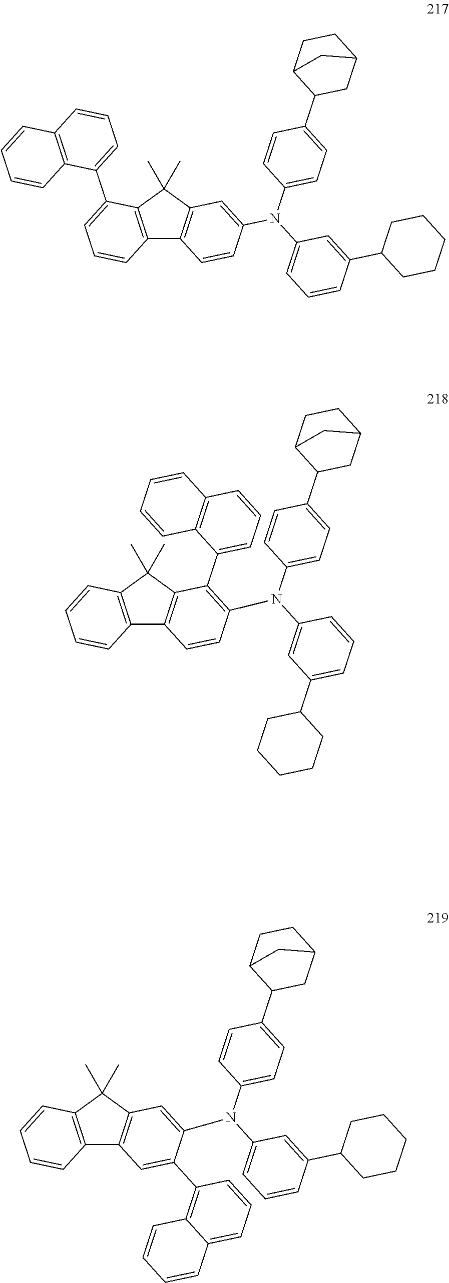 [SMILES: CC1(C)c2cc(N(c3ccc(C4CC5CCC4C5)cc3)c3cccc(C4CCCCC4)c3)ccc2-c2cccc(-c3cccc4ccccc34)c21 ; CC1(C)c2ccccc2-c2ccc(N(c3ccc(C4CC5CCC4C5)cc3)c3cccc(C4CCCCC4)c3)c(-c3cccc4ccccc34)c21 ; CC1(C)c2ccccc2-c2cc(-c3cccc4ccccc34)c(N(c3ccc(C4CC5CCC4C5)cc3)c3cccc(C4CCCCC4)c3)cc21] 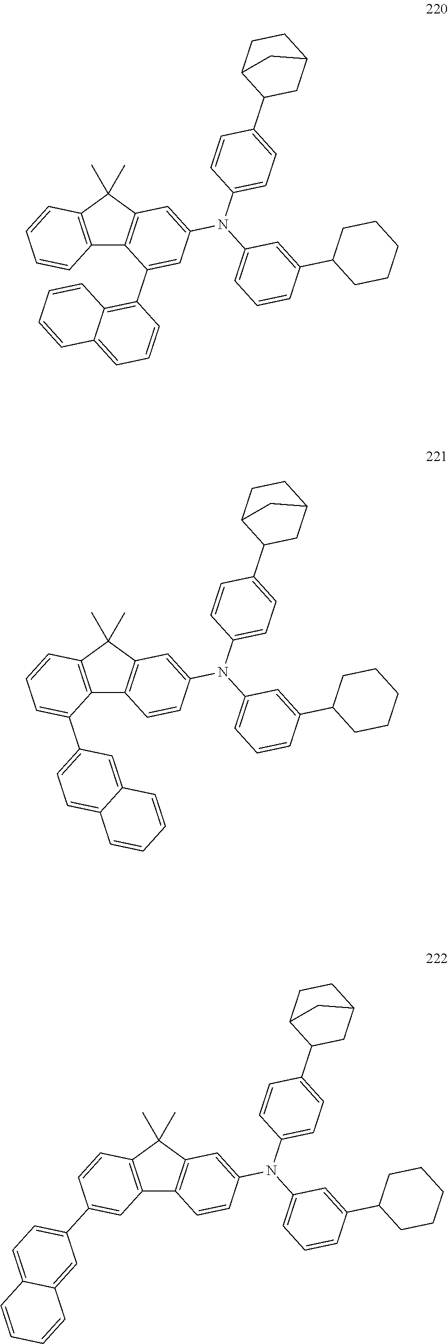 [SMILES: CC1(C)c2ccccc2-c2c(-c3cccc4ccccc34)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3cccc(C4CCCCC4)c3)cc21] 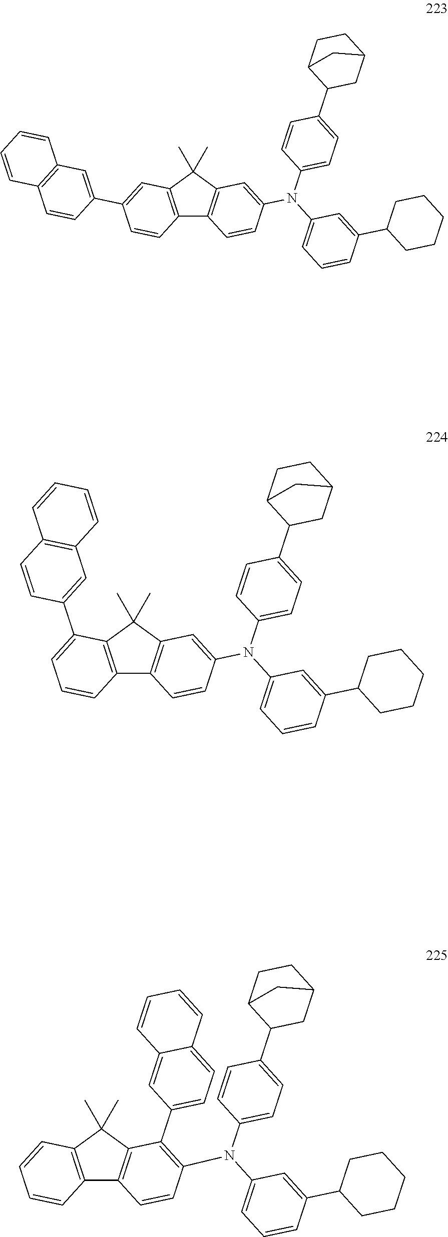 [SMILES: CC1(C)c2cc(-c3ccc4ccccc4c3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc4ccccc4c3)c21 ; CC1(C)c2ccccc2-c2ccc(N(c3ccc(C4CC5CCC4C5)cc3)c3cccc(C4CCCCC4)c3)c(-c3ccc4ccccc4c3)c21] 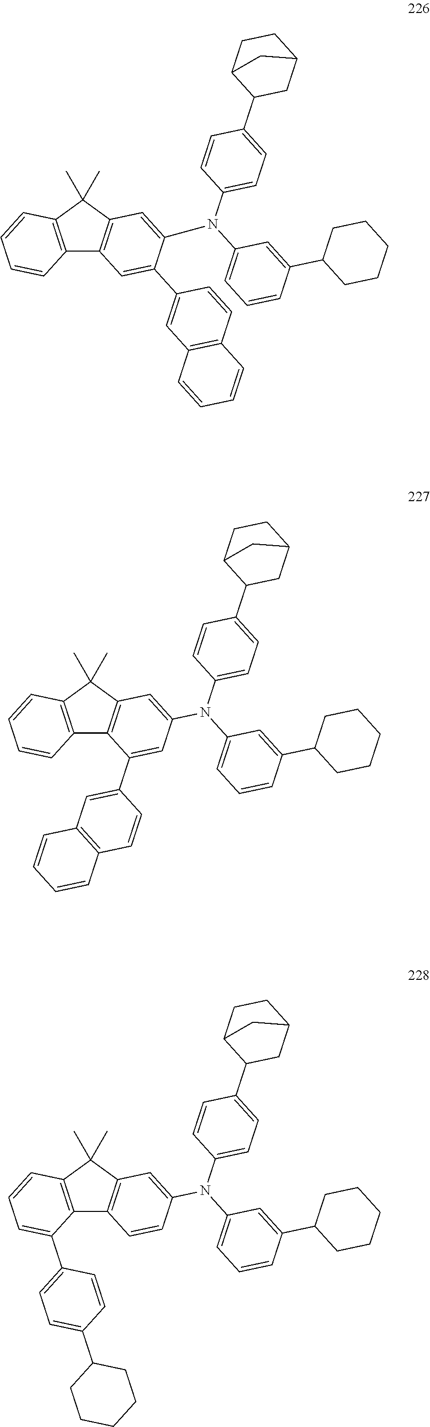 [SMILES: CC1(C)c2ccccc2-c2cc(-c3ccc4ccccc4c3)c(N(c3ccc(C4CC5CCC4C5)cc3)c3cccc(C4CCCCC4)c3)cc21 ; CC1(C)c2ccccc2-c2c(-c3ccc4ccccc4c3)cc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2c(-c3ccc(C4CCCCC4)cc3)cccc21] 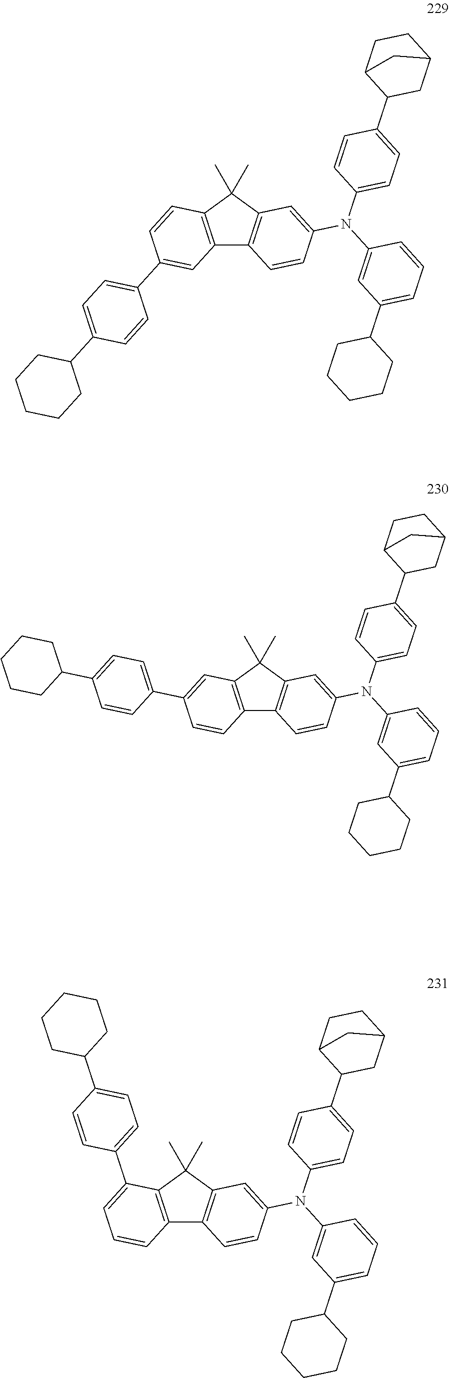 [SMILES: CC1(C)c2ccc(-c3ccc(C4CCCCC4)cc3)cc2-c2ccc(N(c3ccc(C4CC5CCC4C5)cc3)c3cccc(C4CCCCC4)c3)cc21 ; CC1(C)c2cc(-c3ccc(C4CCCCC4)cc3)ccc2-c2ccc(N(c3ccc(C4CC5CCC4C5)cc3)c3cccc(C4CCCCC4)c3)cc21 ; CC1(C)c2cc(N(c3ccc(C4CC5CCC4C5)cc3)c3cccc(C4CCCCC4)c3)ccc2-c2cccc(-c3ccc(C4CCCCC4)cc3)c21] 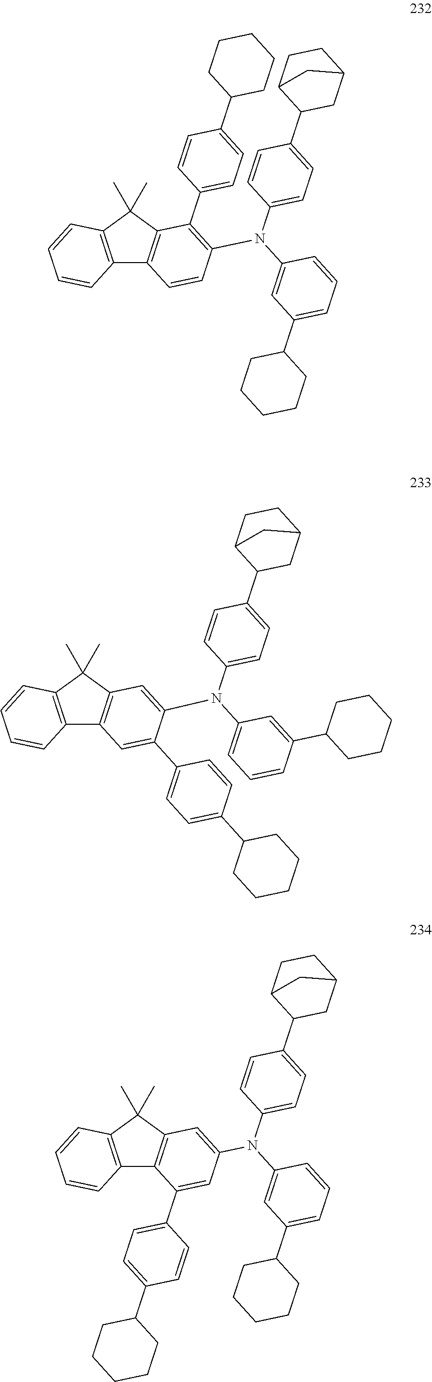 [SMILES: CC1(C)c2ccccc2-c2ccc(N(c3ccc(C4CC5CCC4C5)cc3)c3cccc(C4CCCCC4)c3)c(-c3ccc(C4CCCCC4)cc3)c21 ; CC1(C)c2ccccc2-c2cc(-c3ccc(C4CCCCC4)cc3)c(N(c3ccc(C4CC5CCC4C5)cc3)c3cccc(C4CCCCC4)c3)cc21 ; CC1(C)c2ccccc2-c2c(-c3ccc(C4CCCCC4)cc3)cc(N(c3ccc(C4CC5CCC4C5)cc3)c3cccc(C4CCCCC4)c3)cc21] 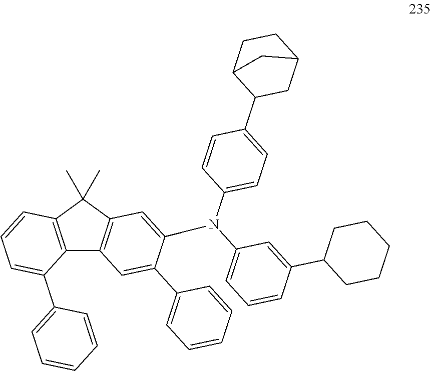 [SMILES: CC1(C)c2cc(N(c3ccc(C4CC5CCC4C5)cc3)c3cccc(C4CCCCC4)c3)c(-c3ccccc3)cc2-c2c(-c3ccccc3)cccc21] 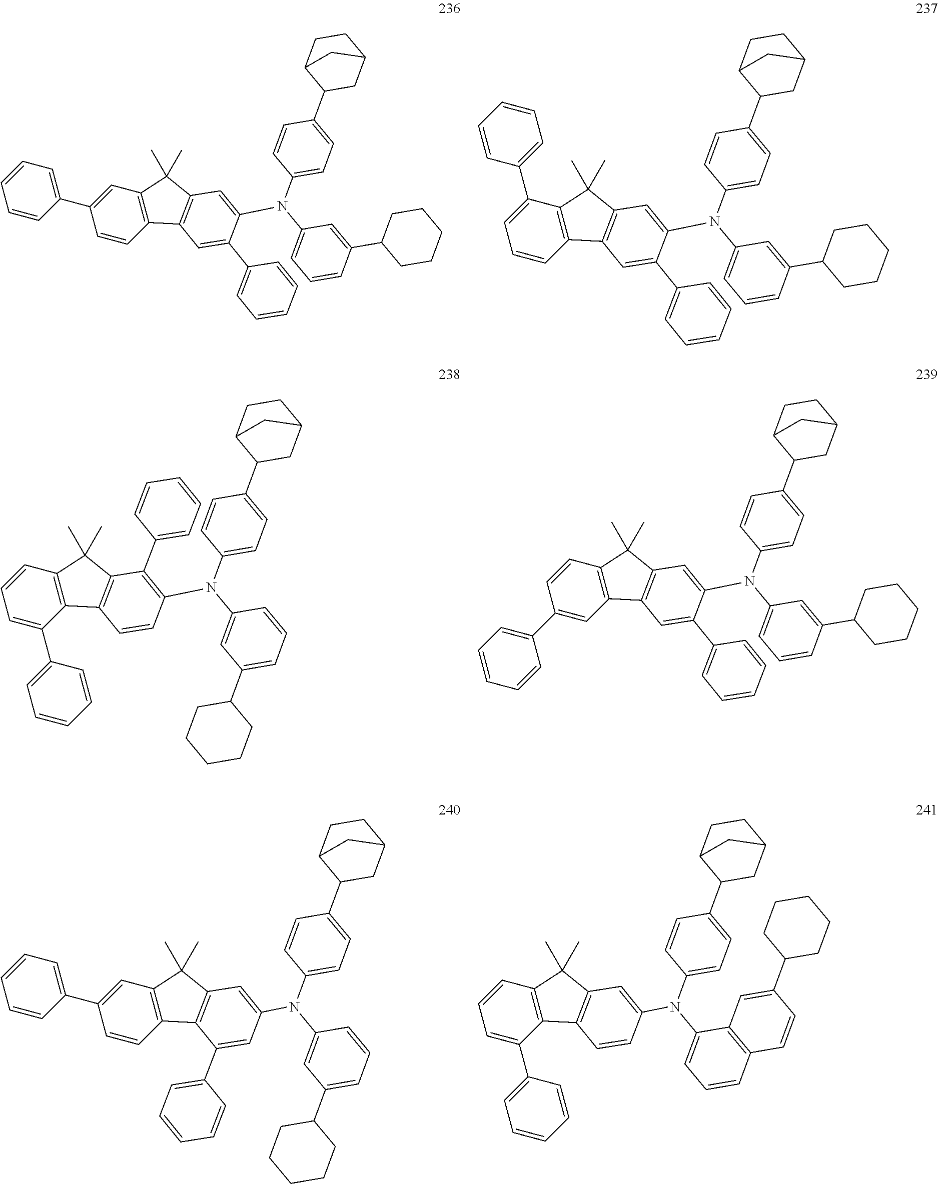 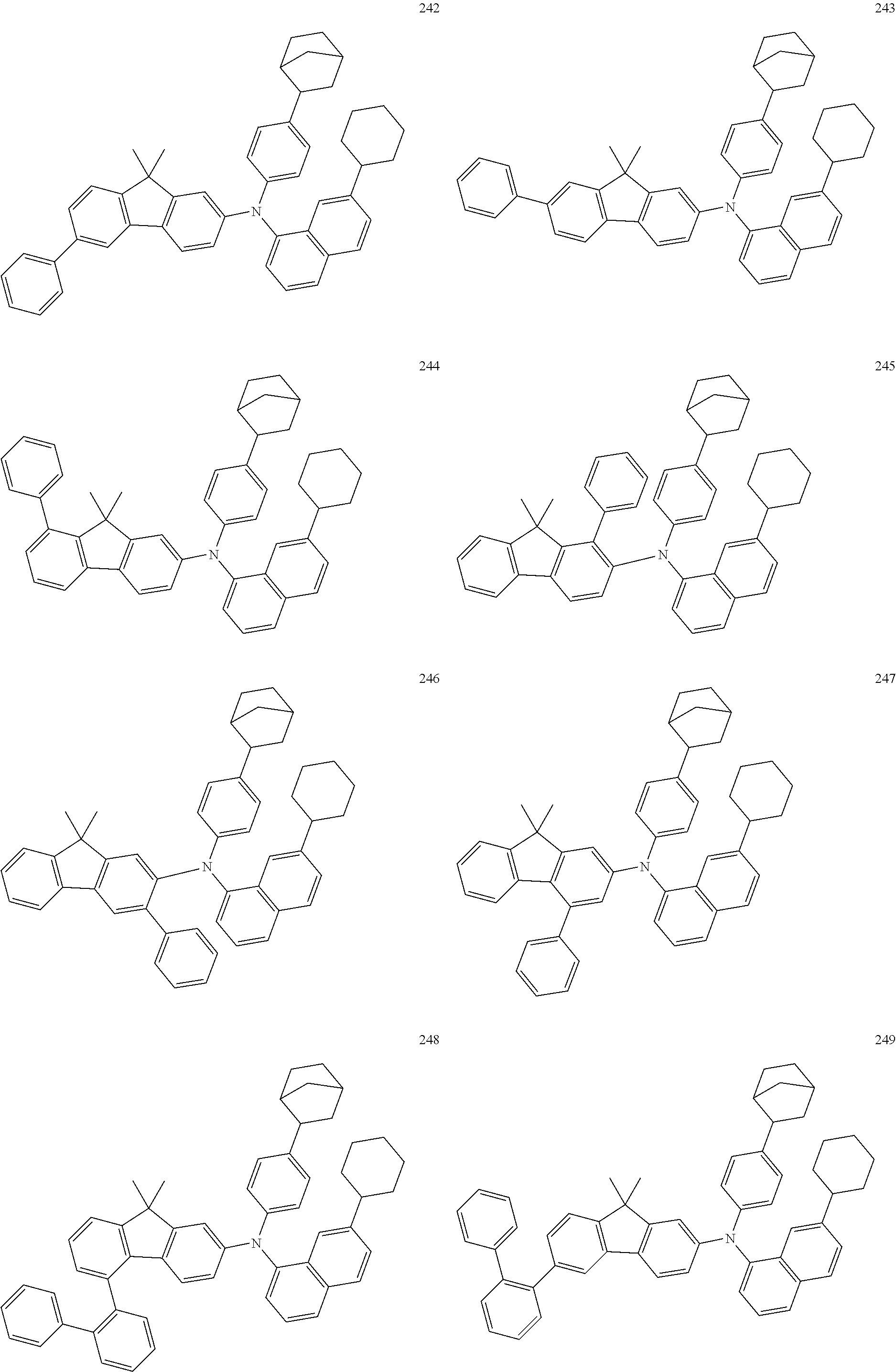 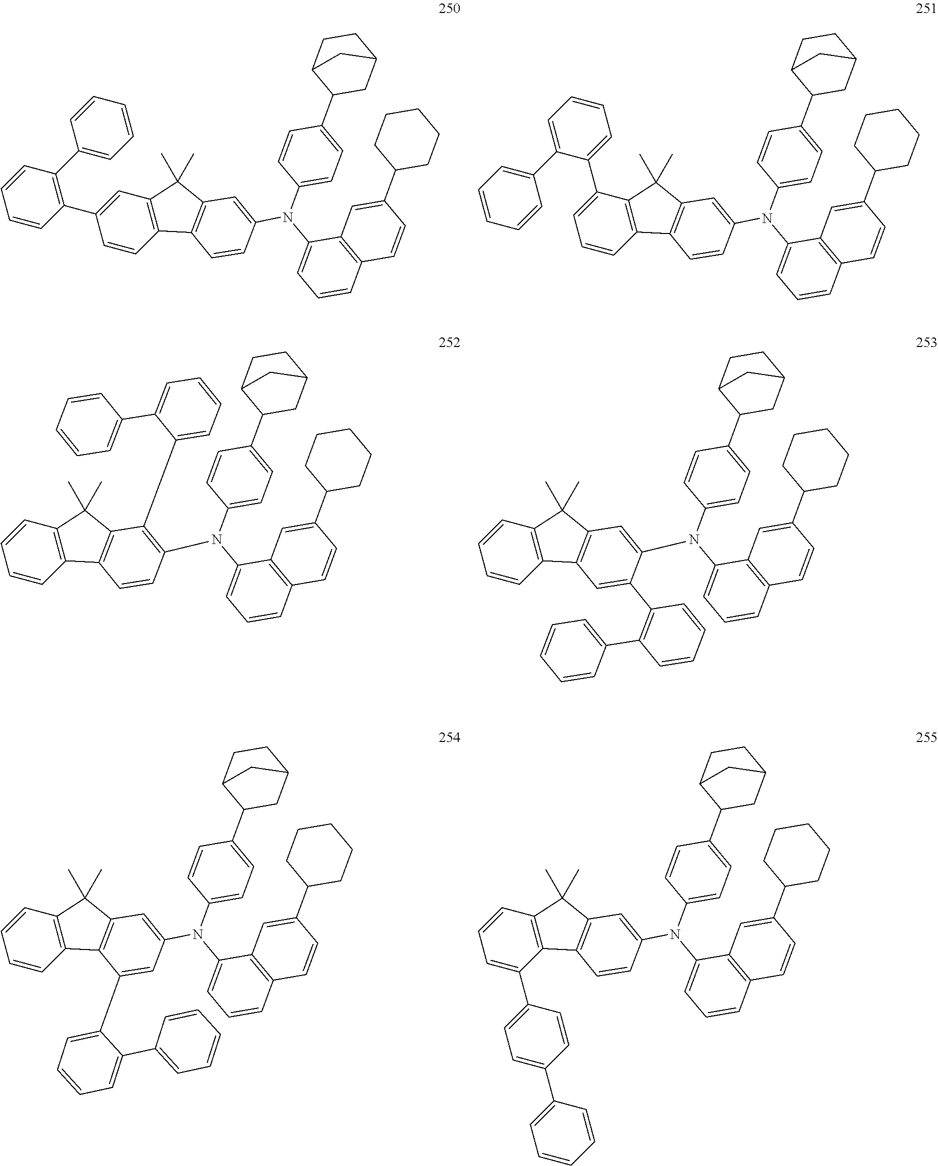 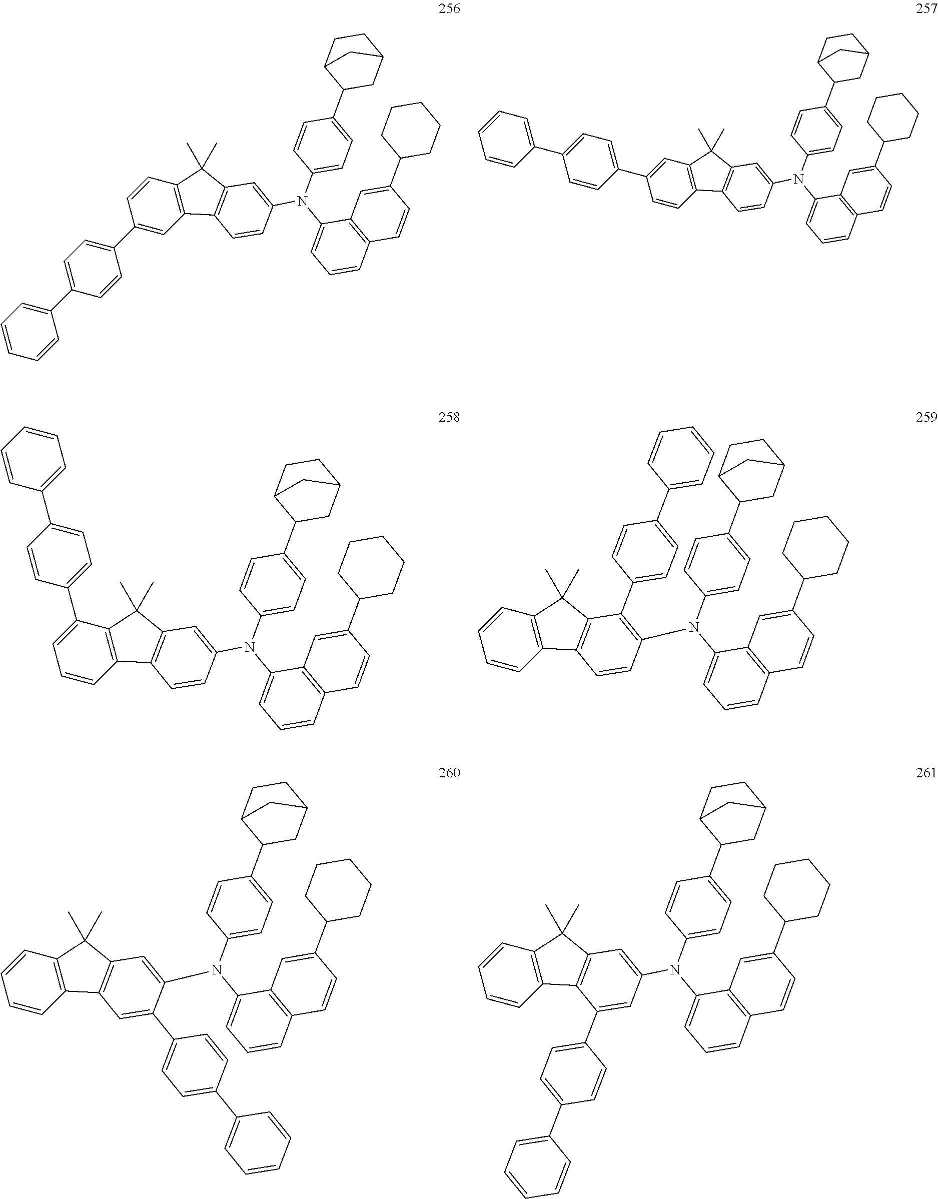 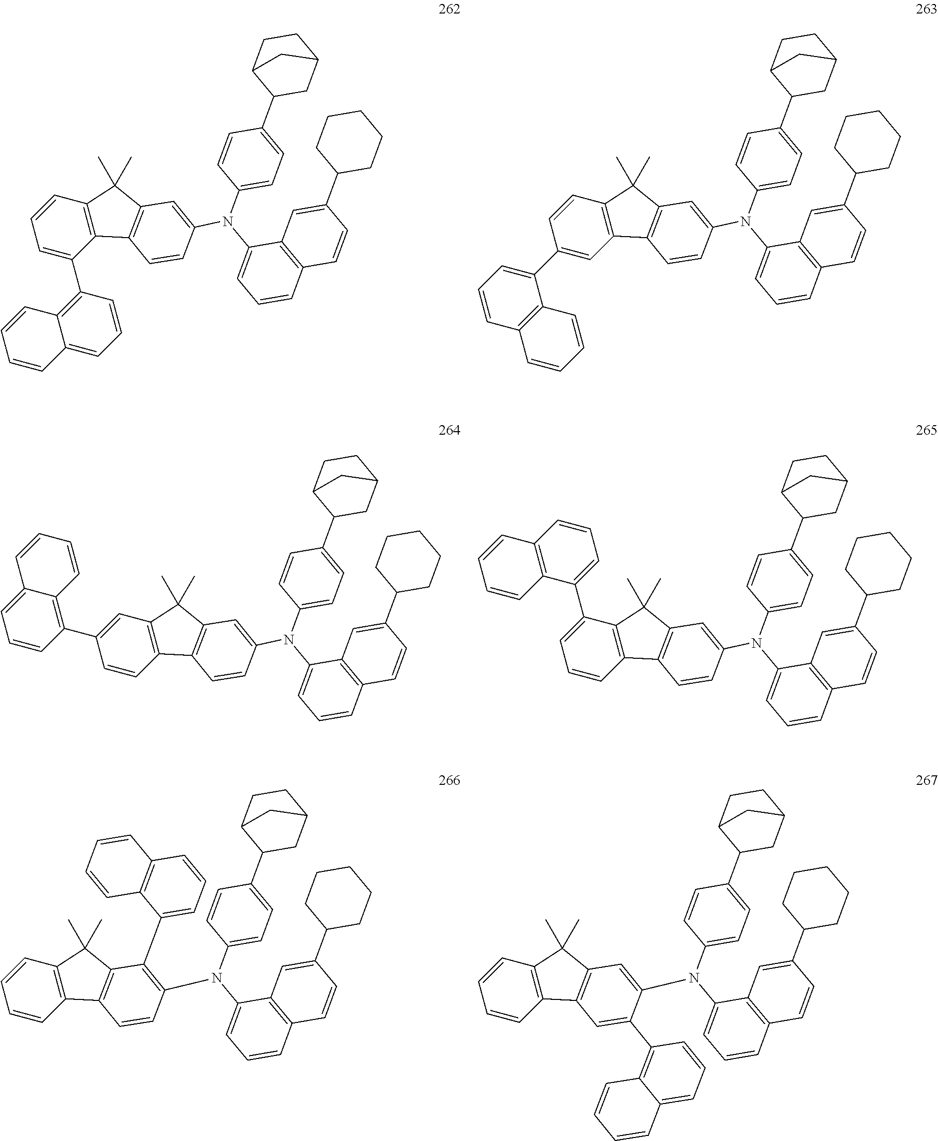 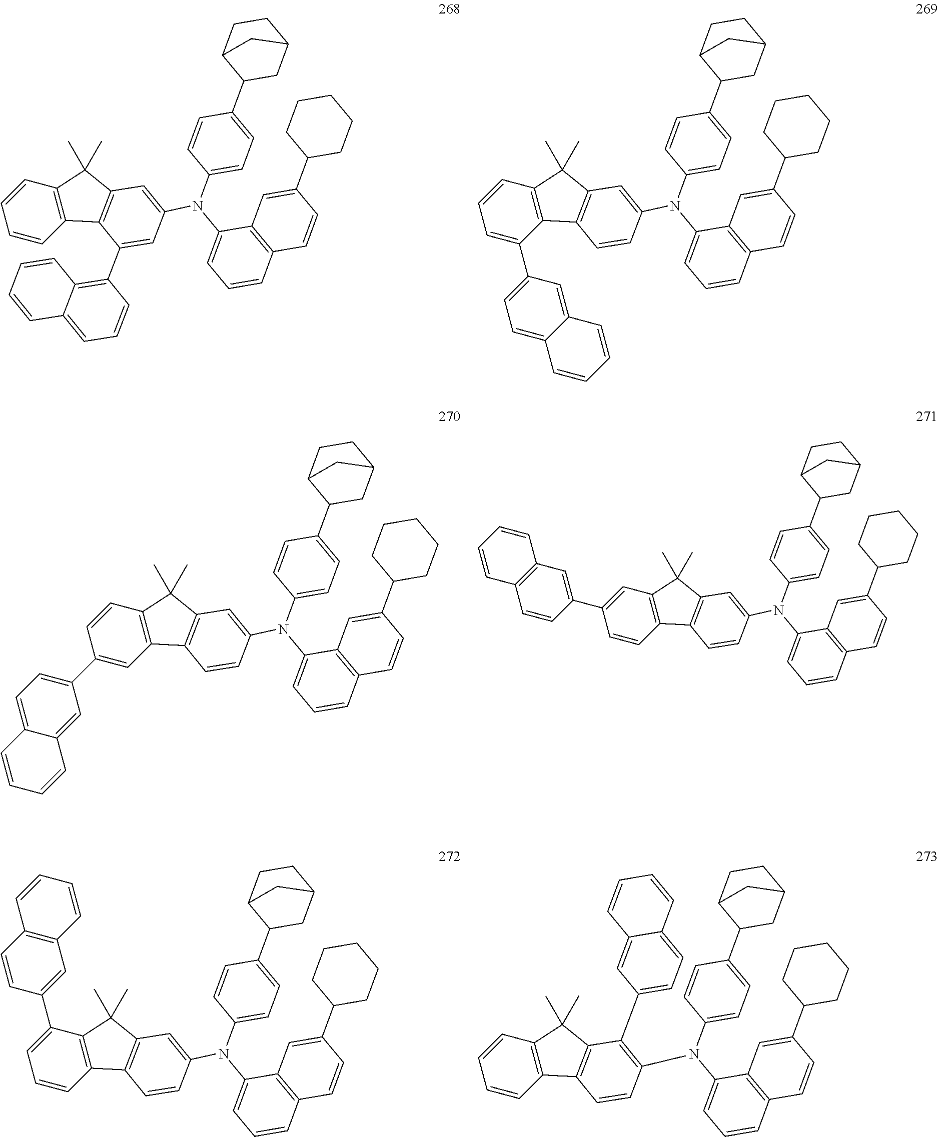 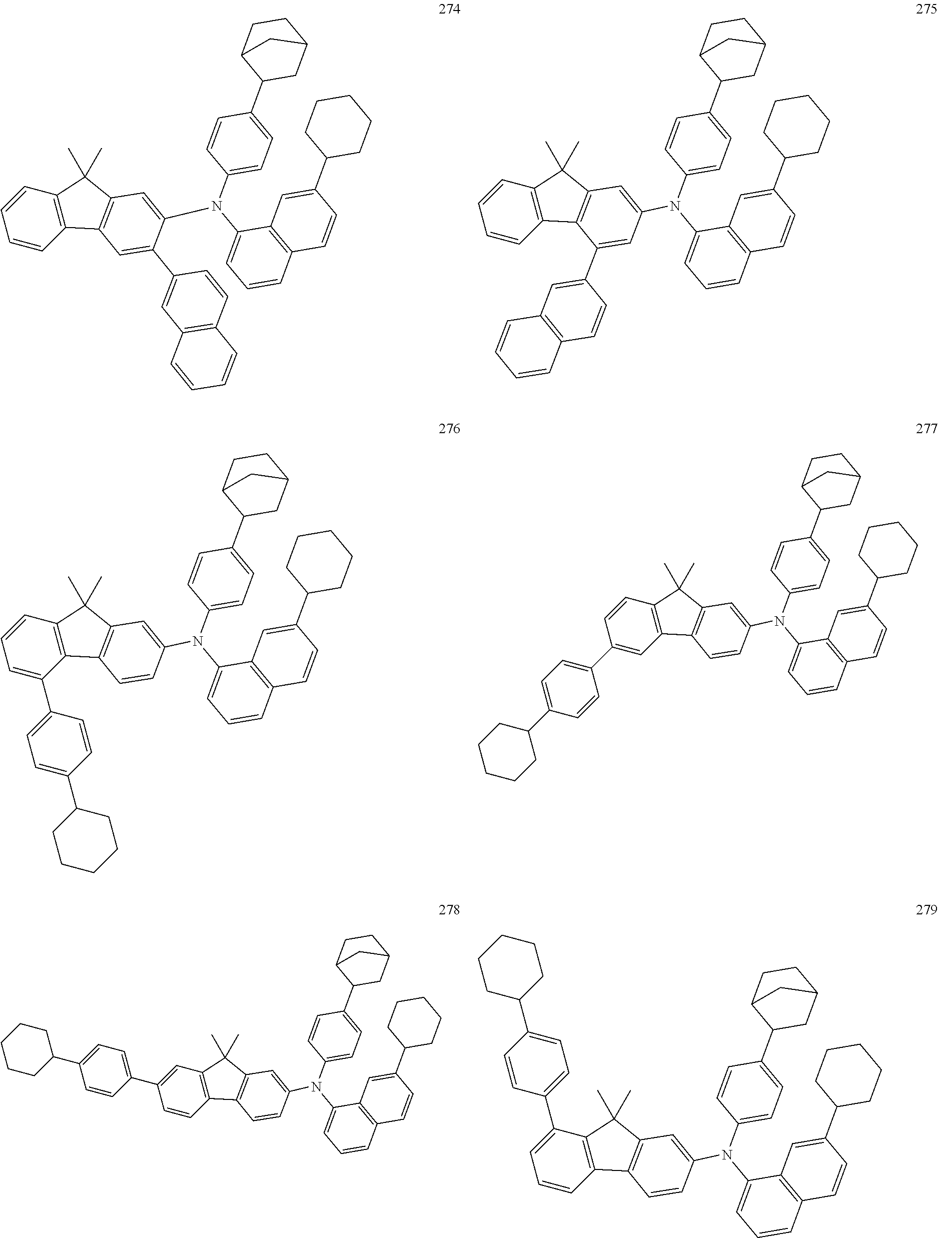 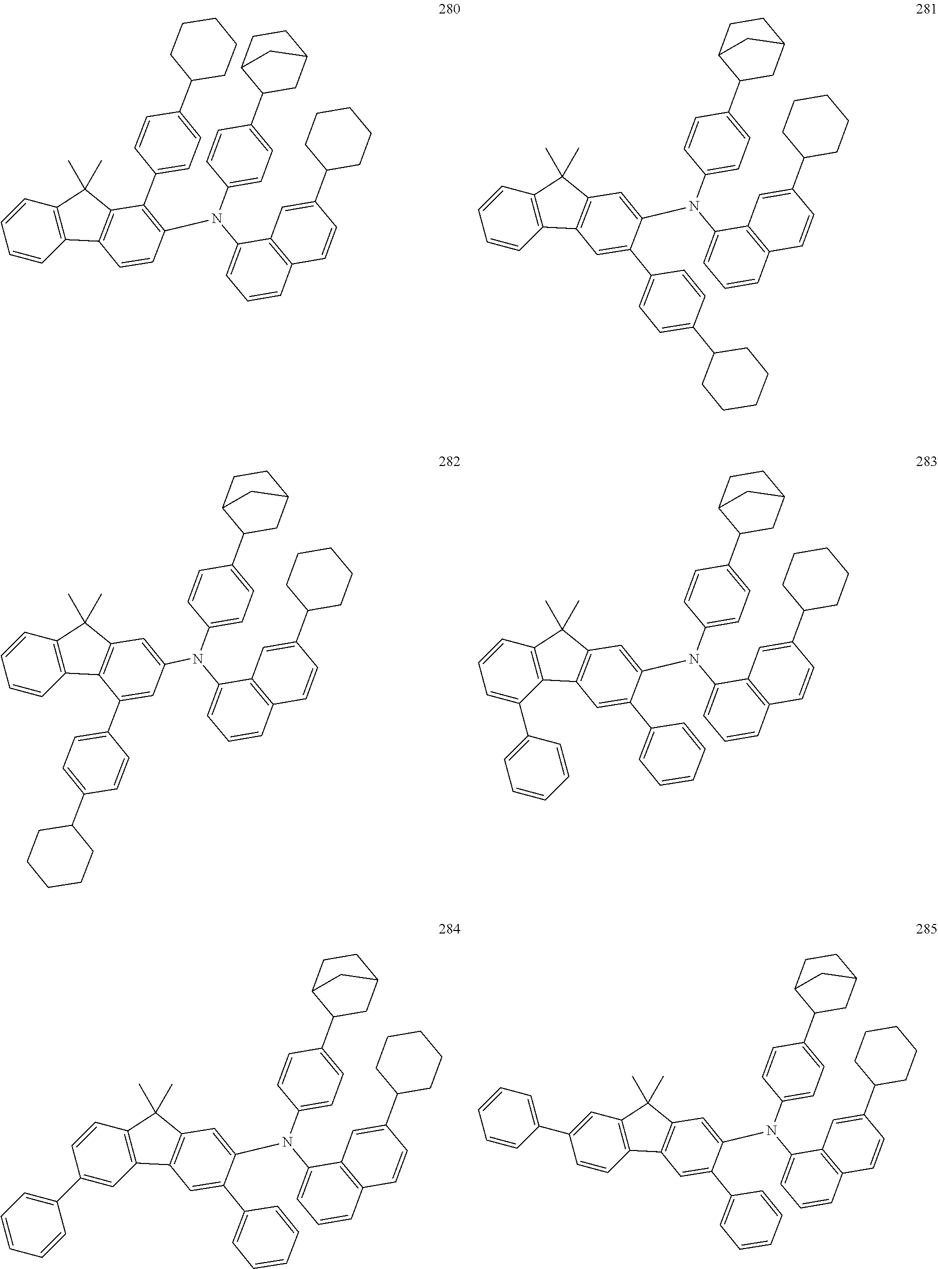 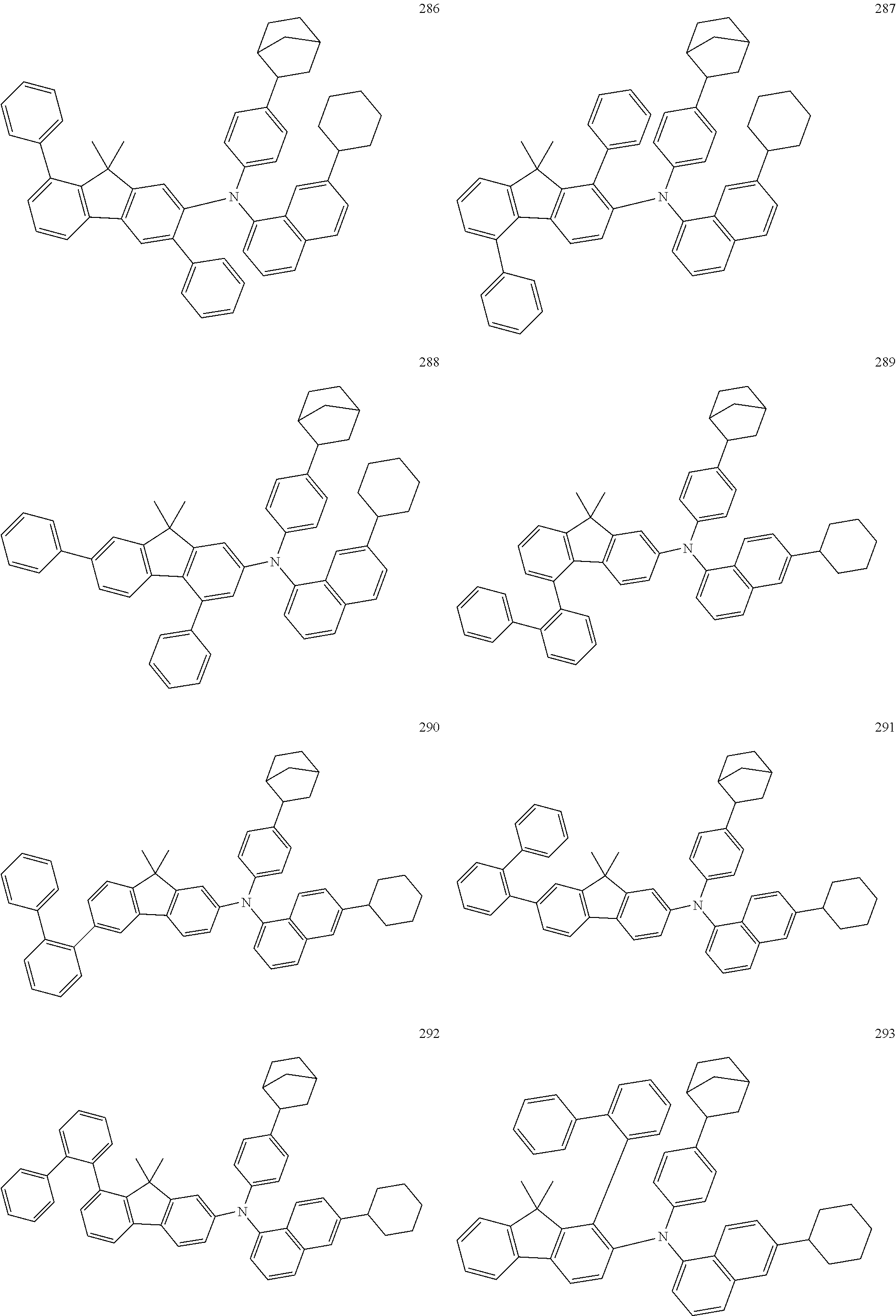 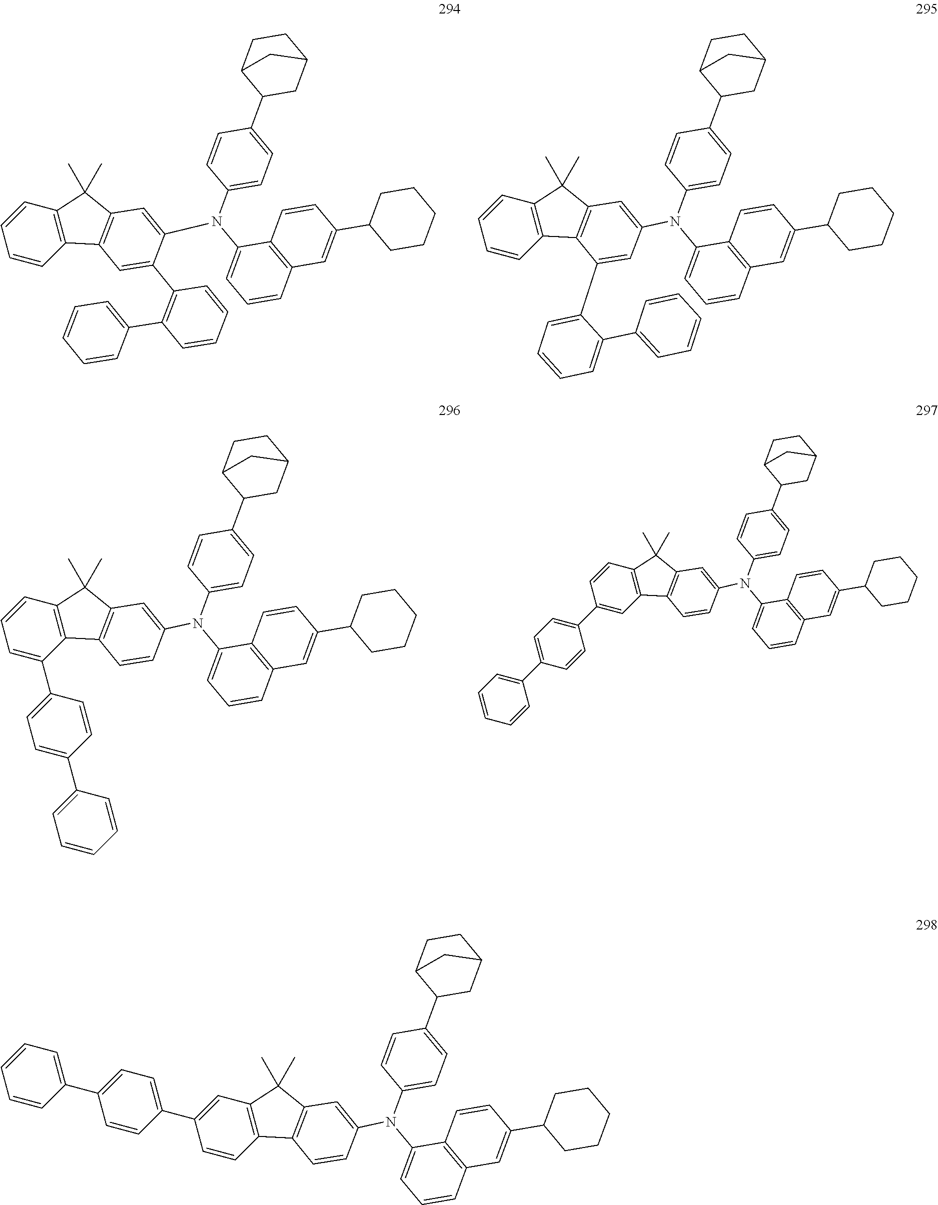 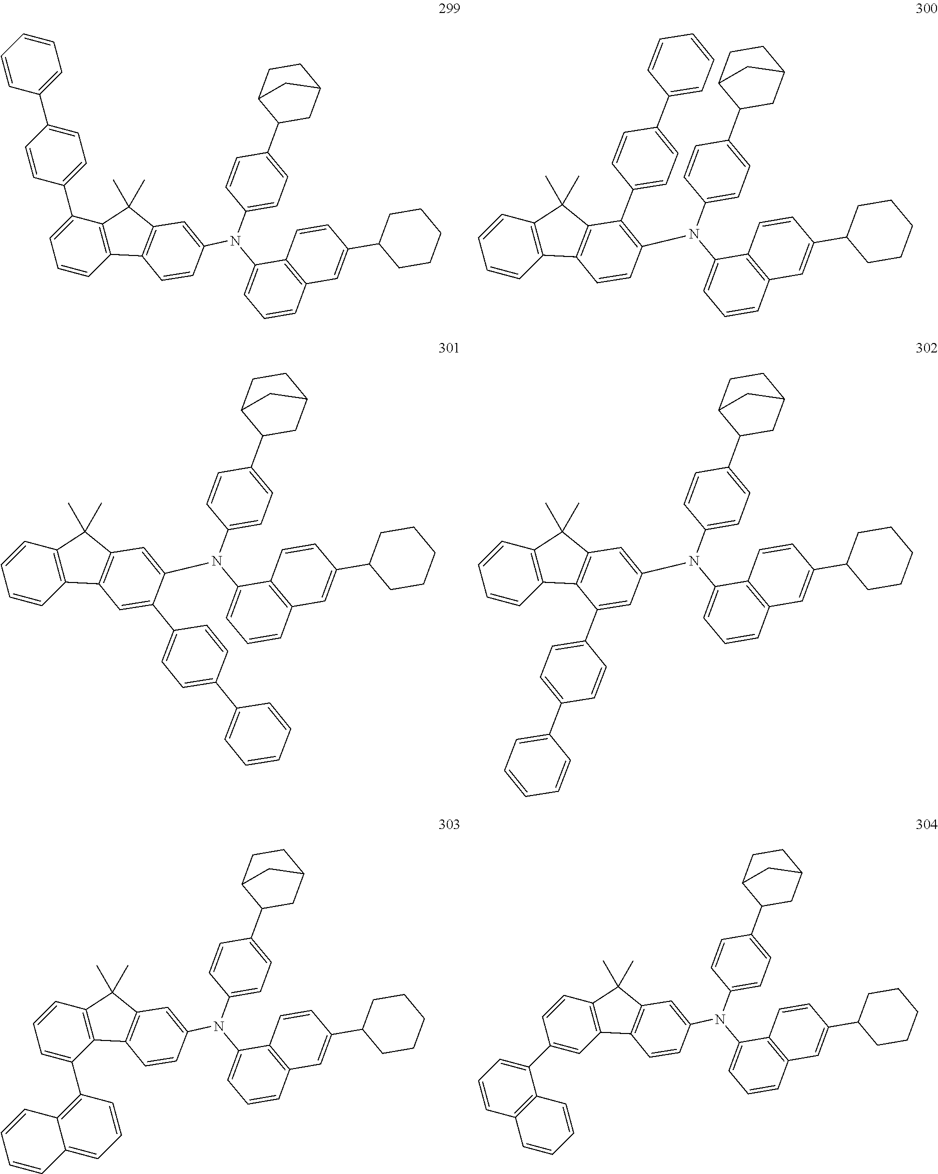 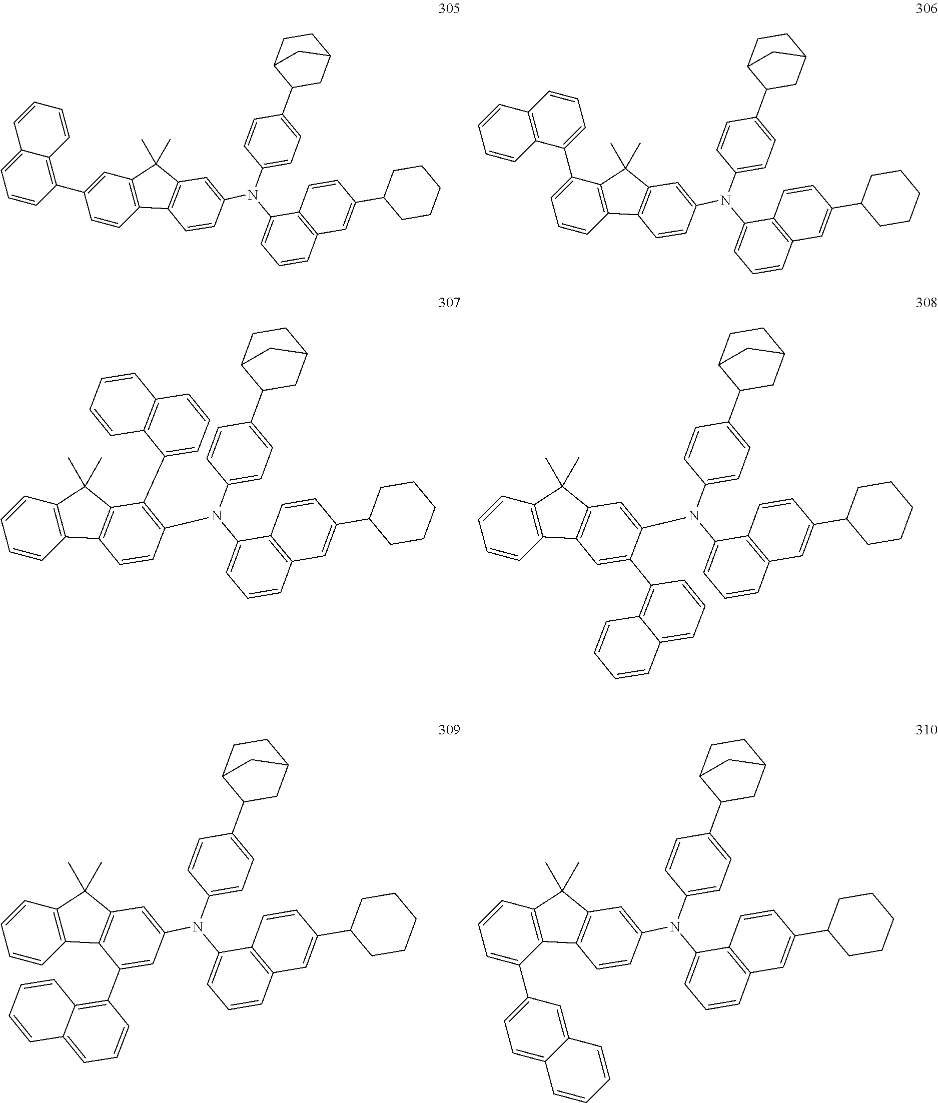 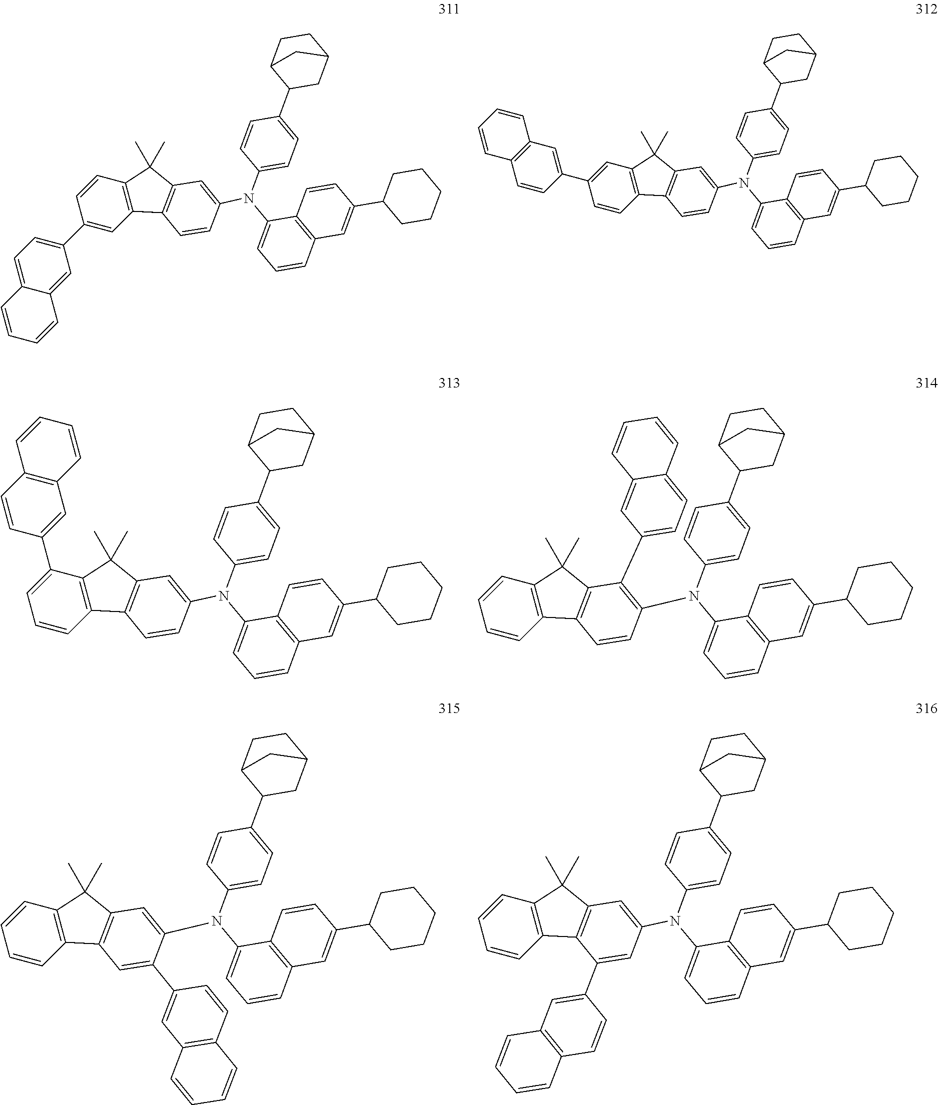 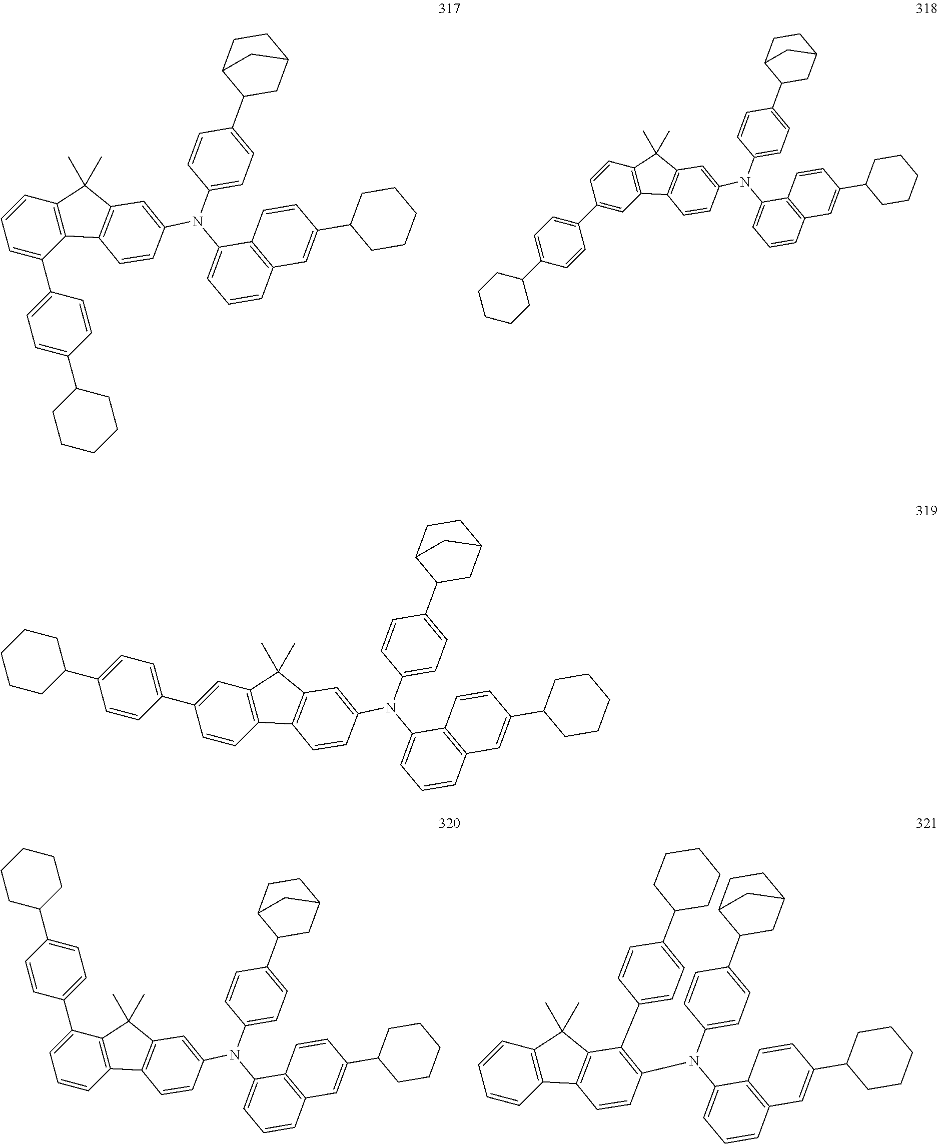 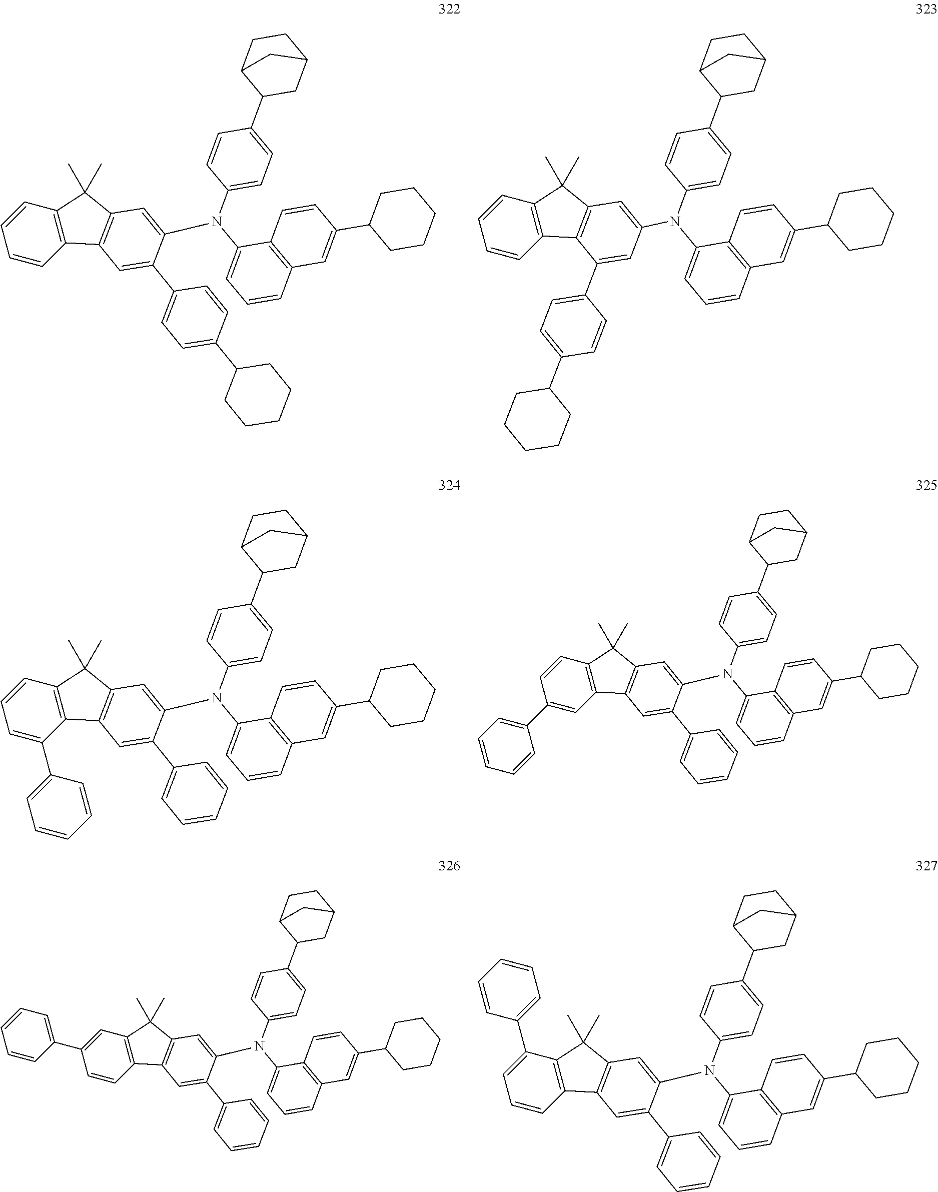 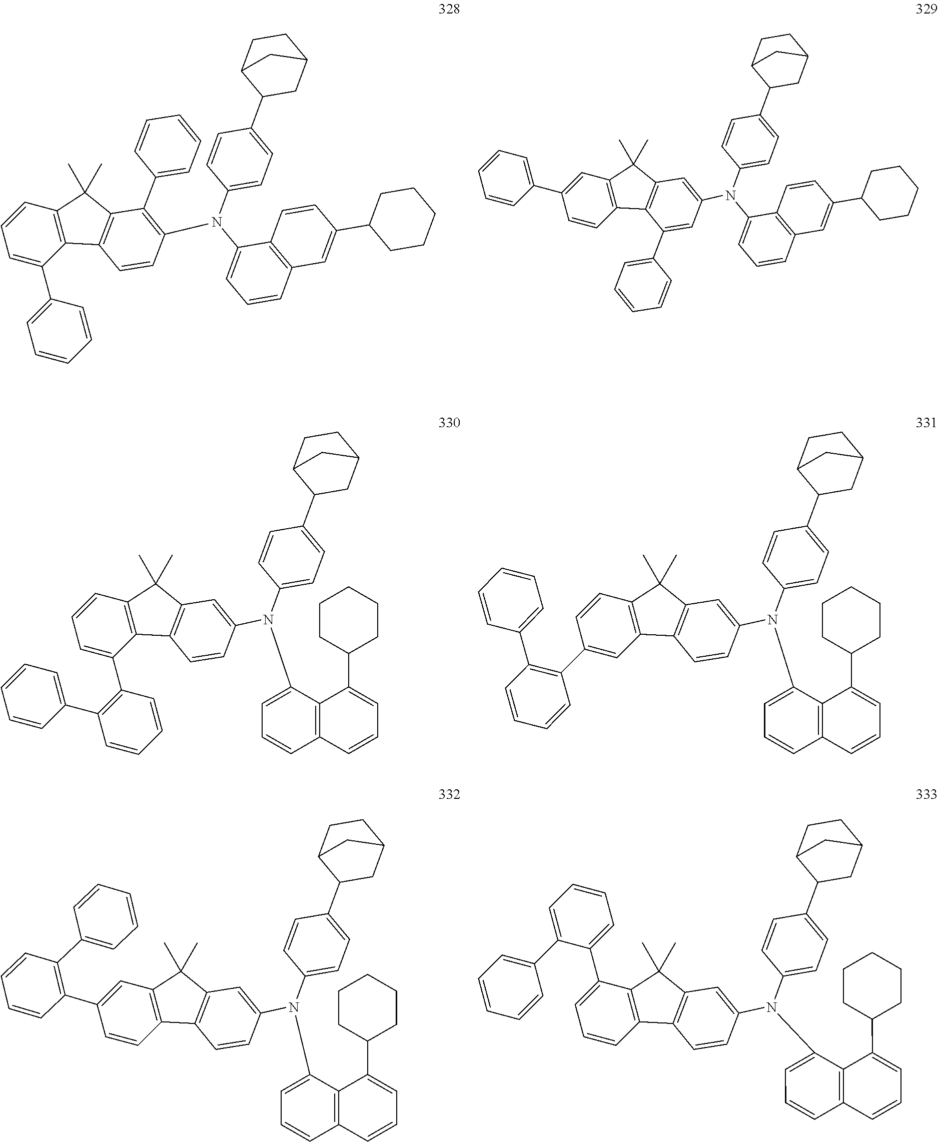 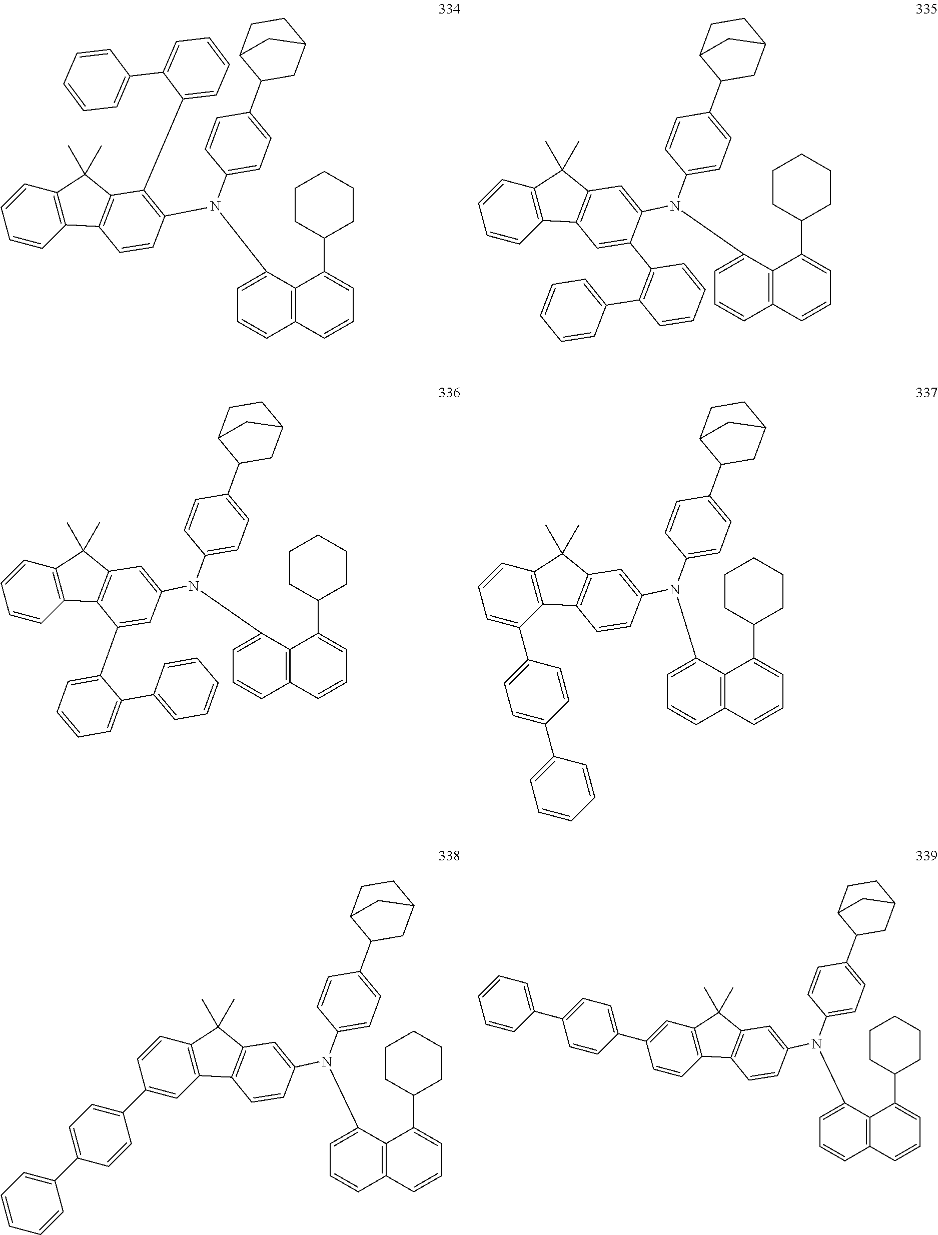 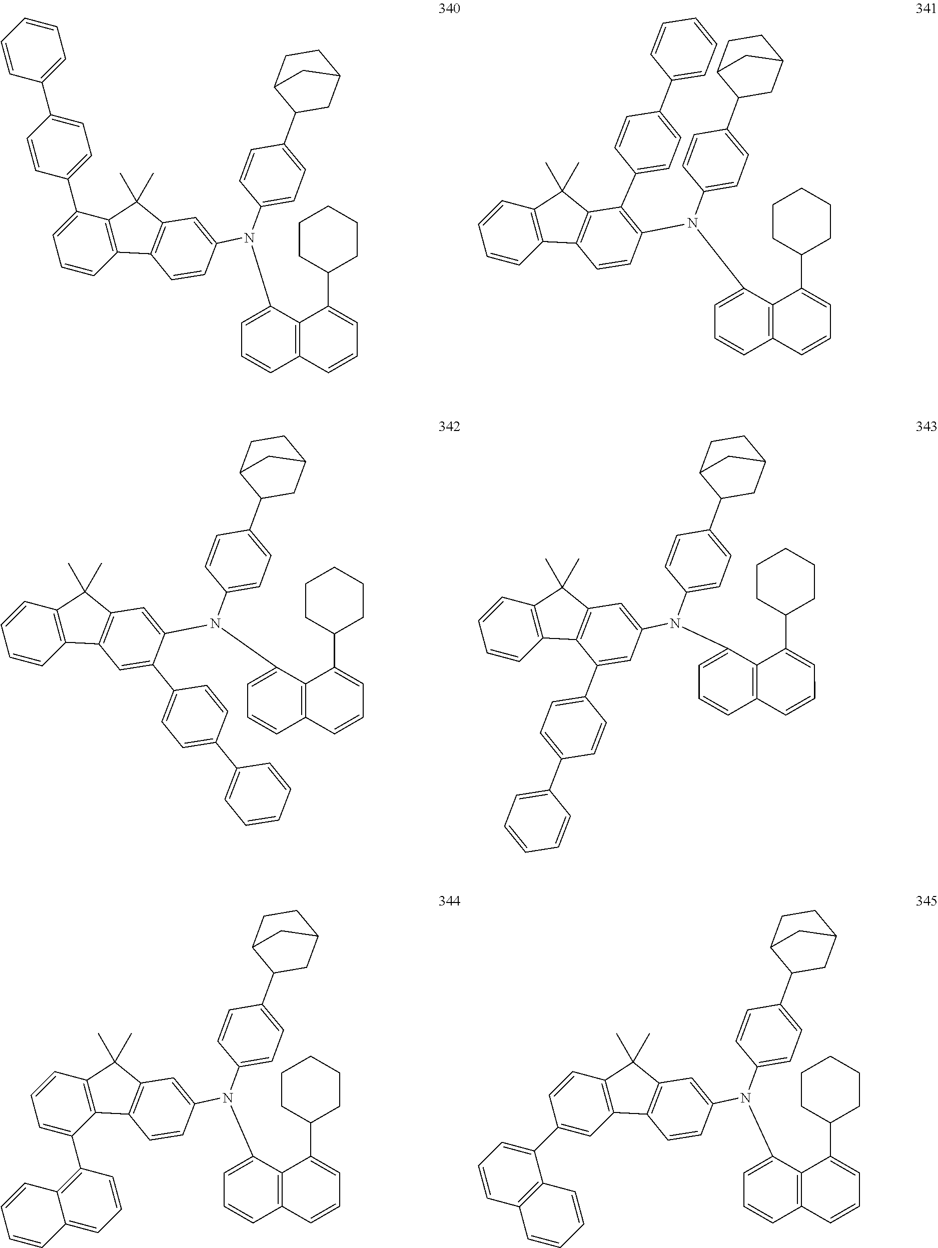 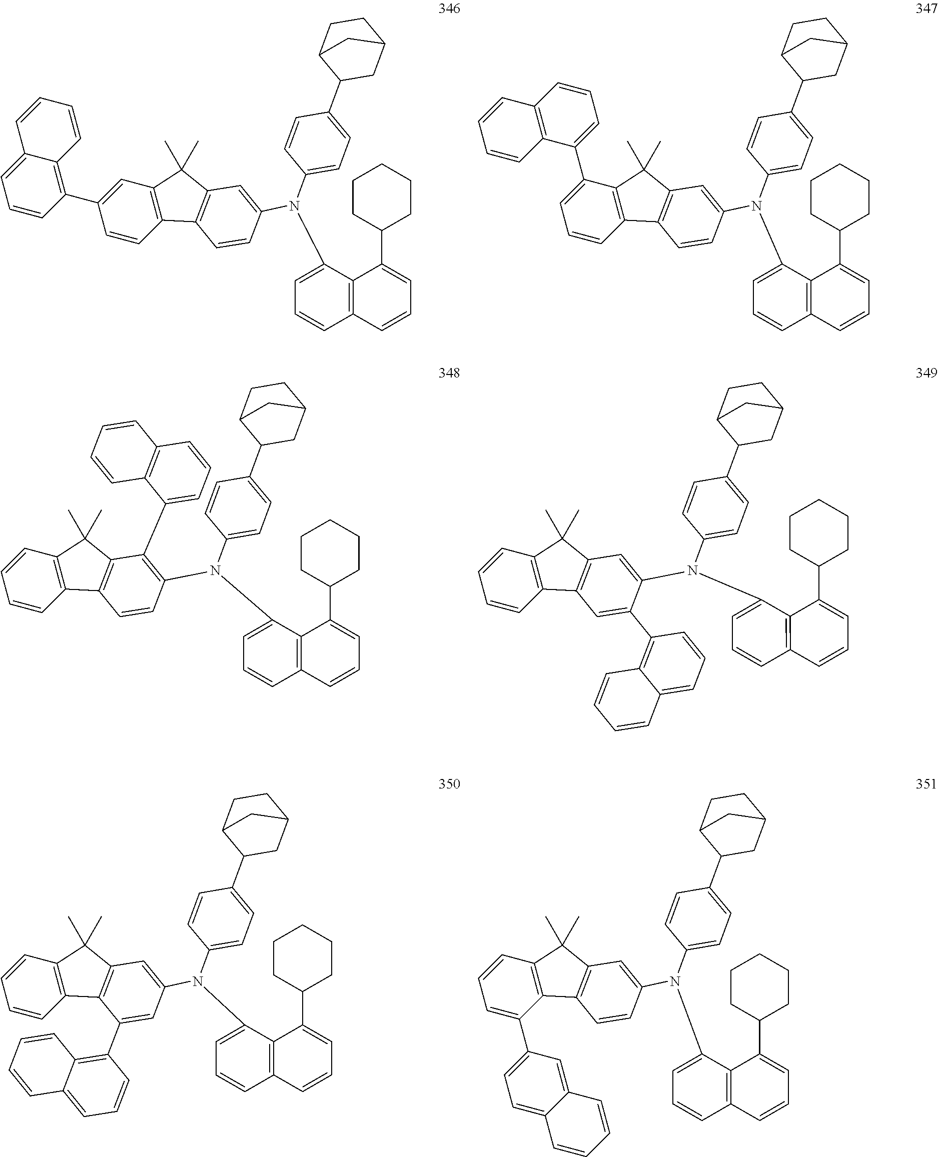 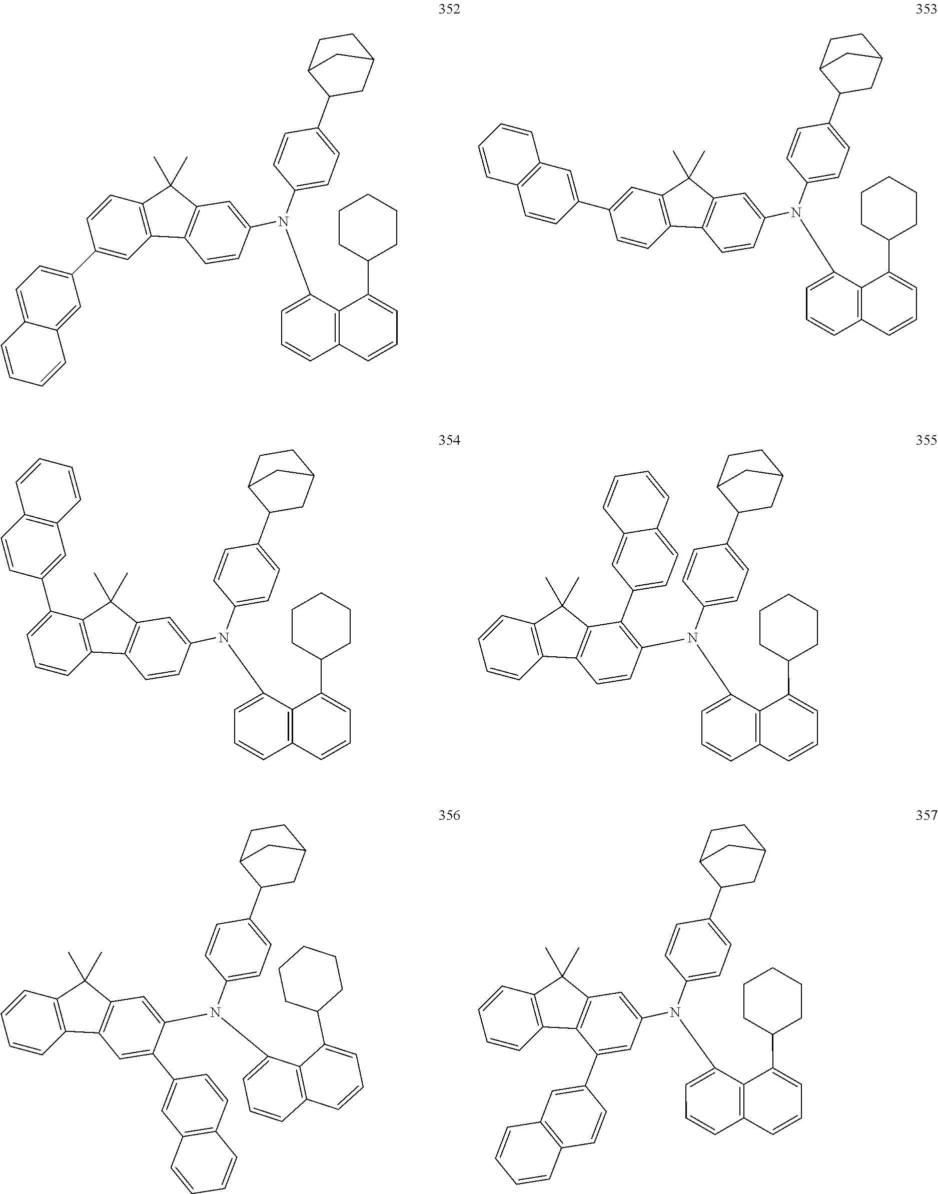 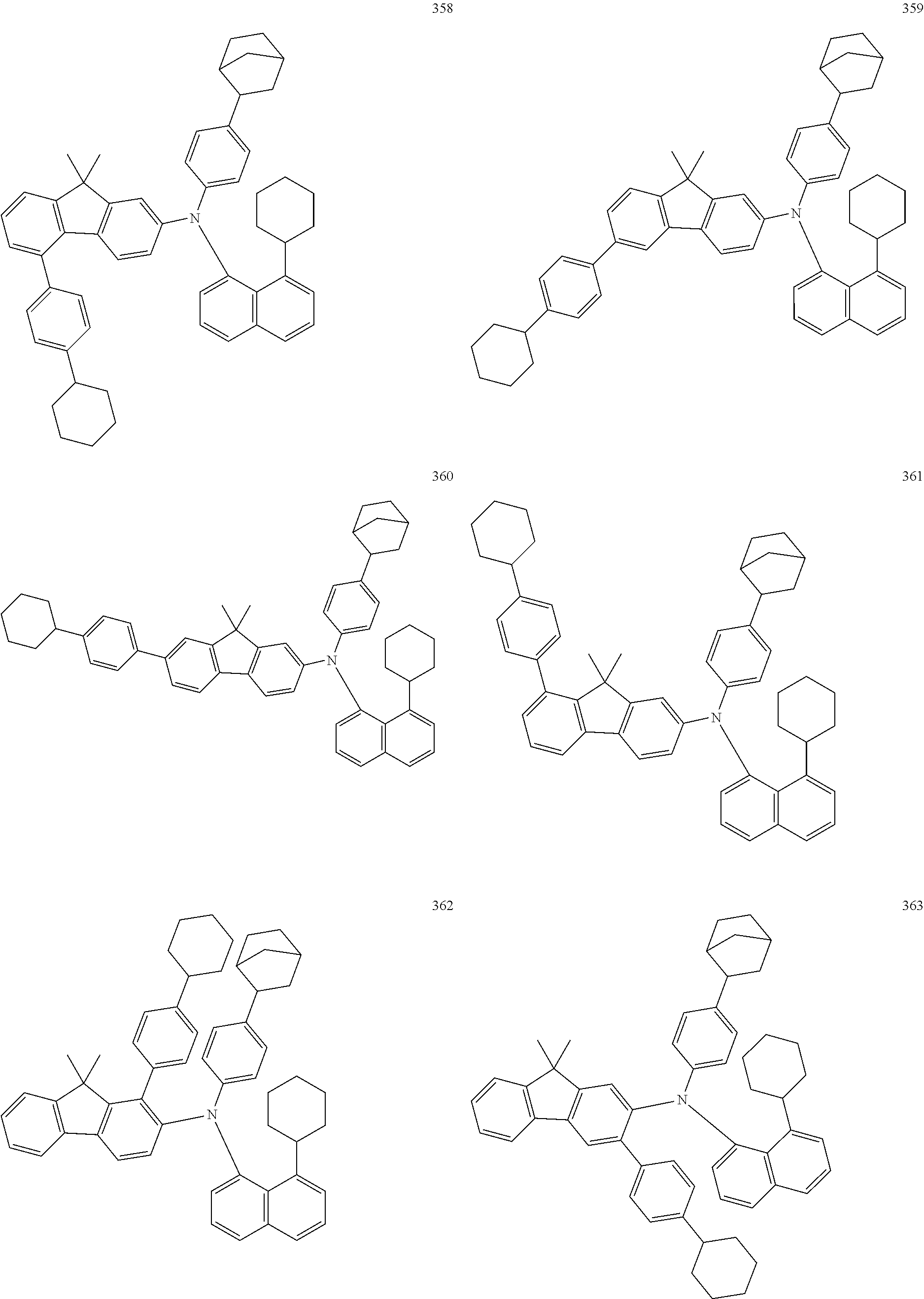 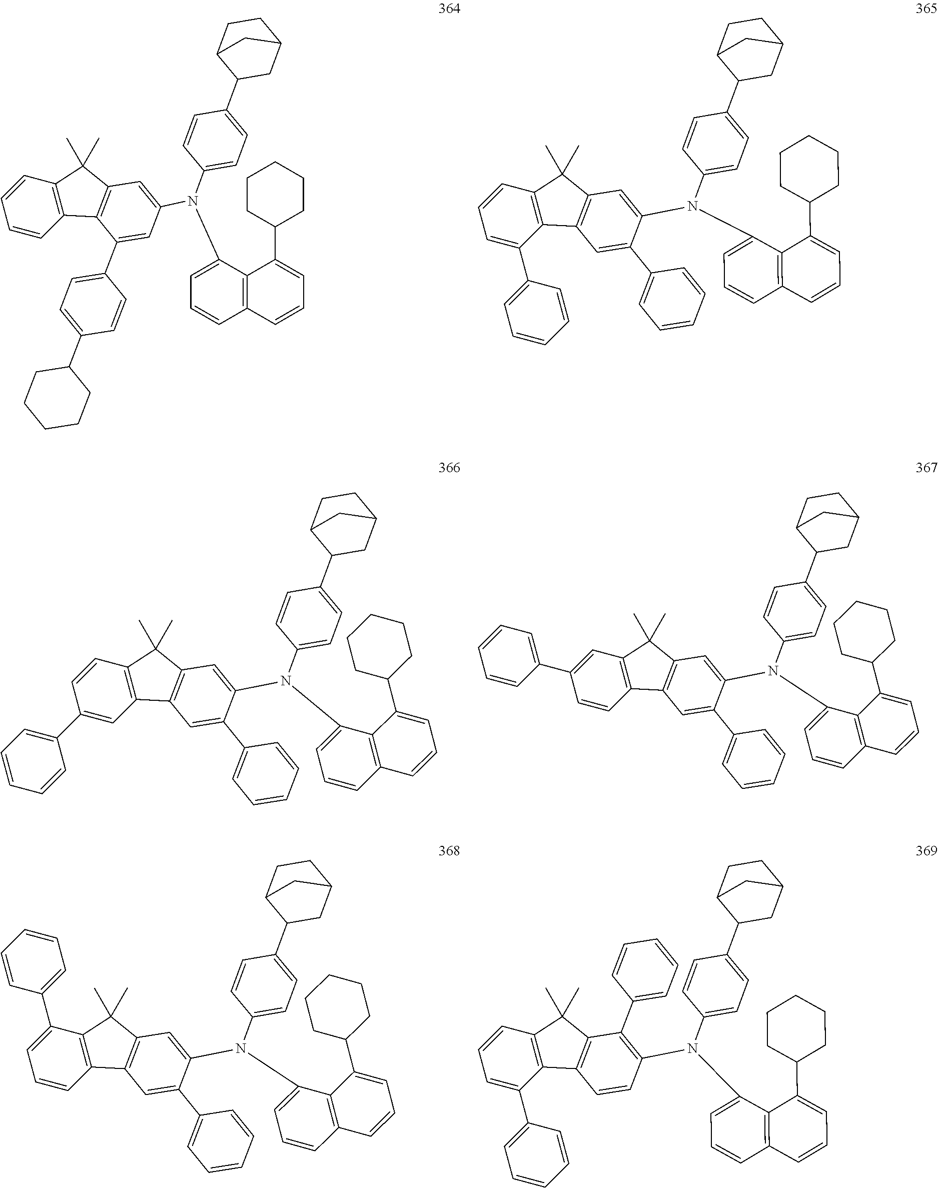 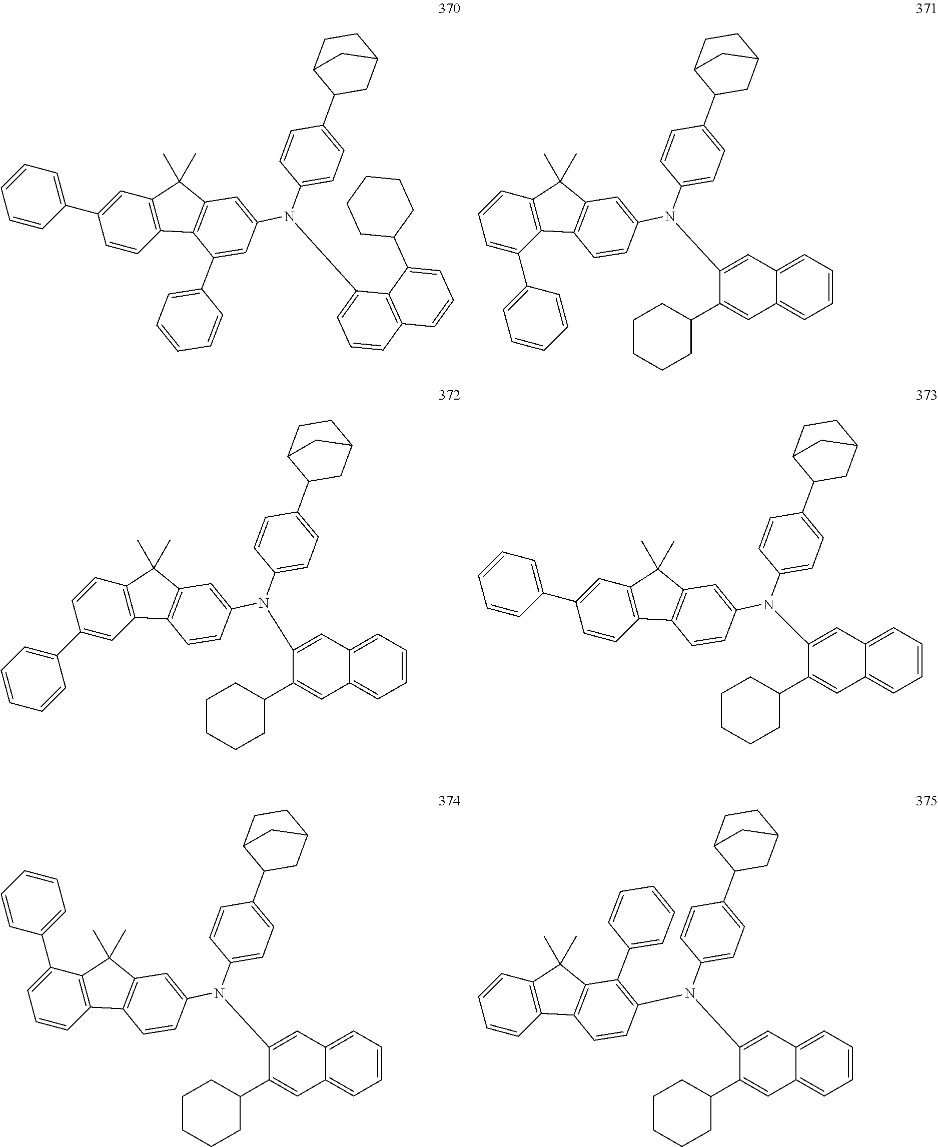 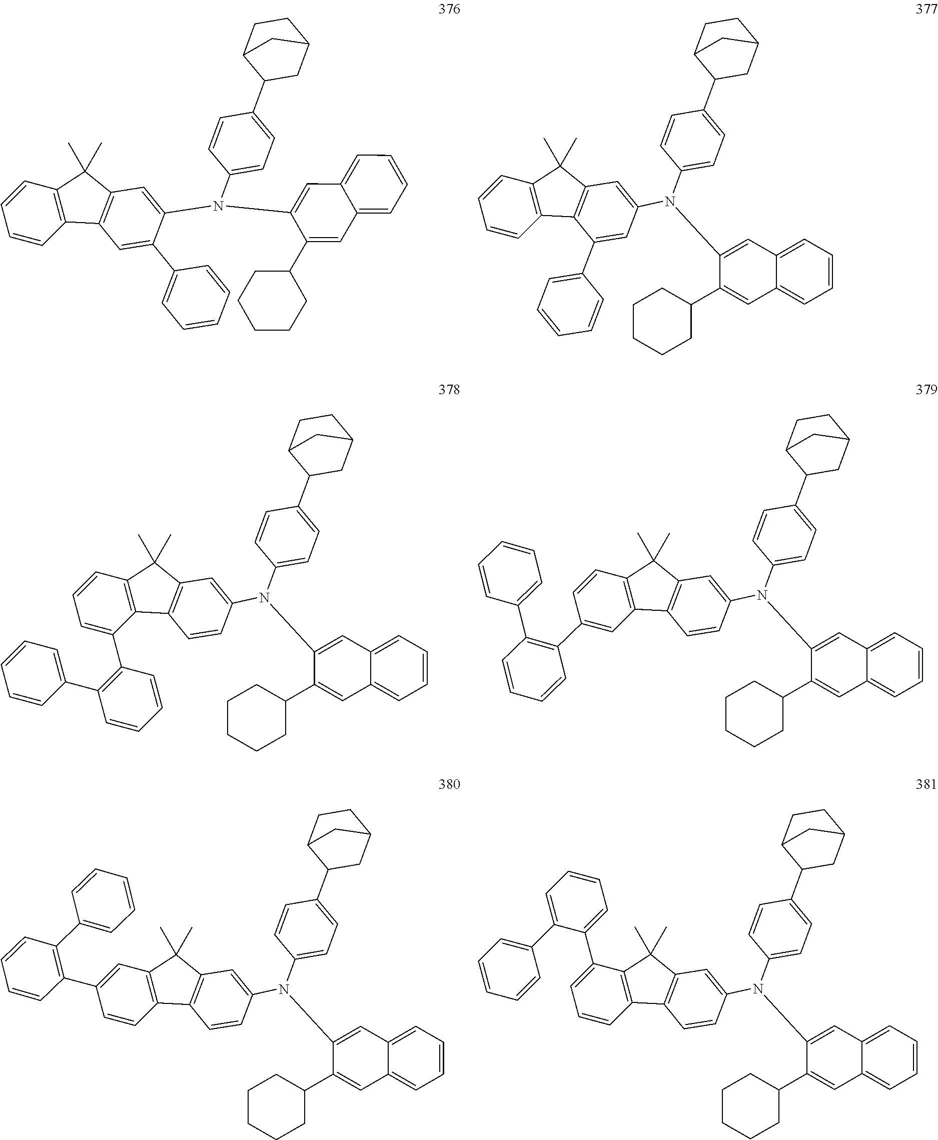 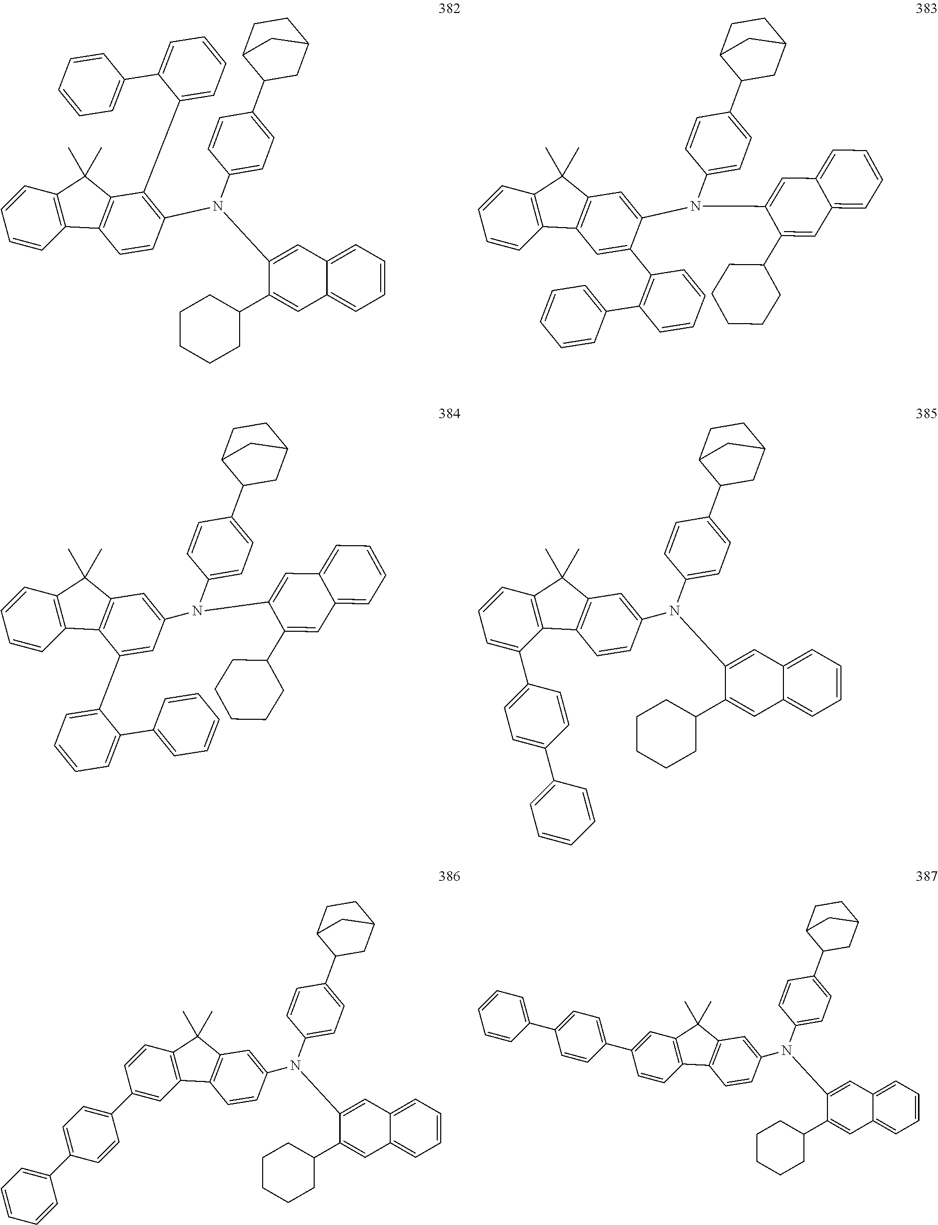 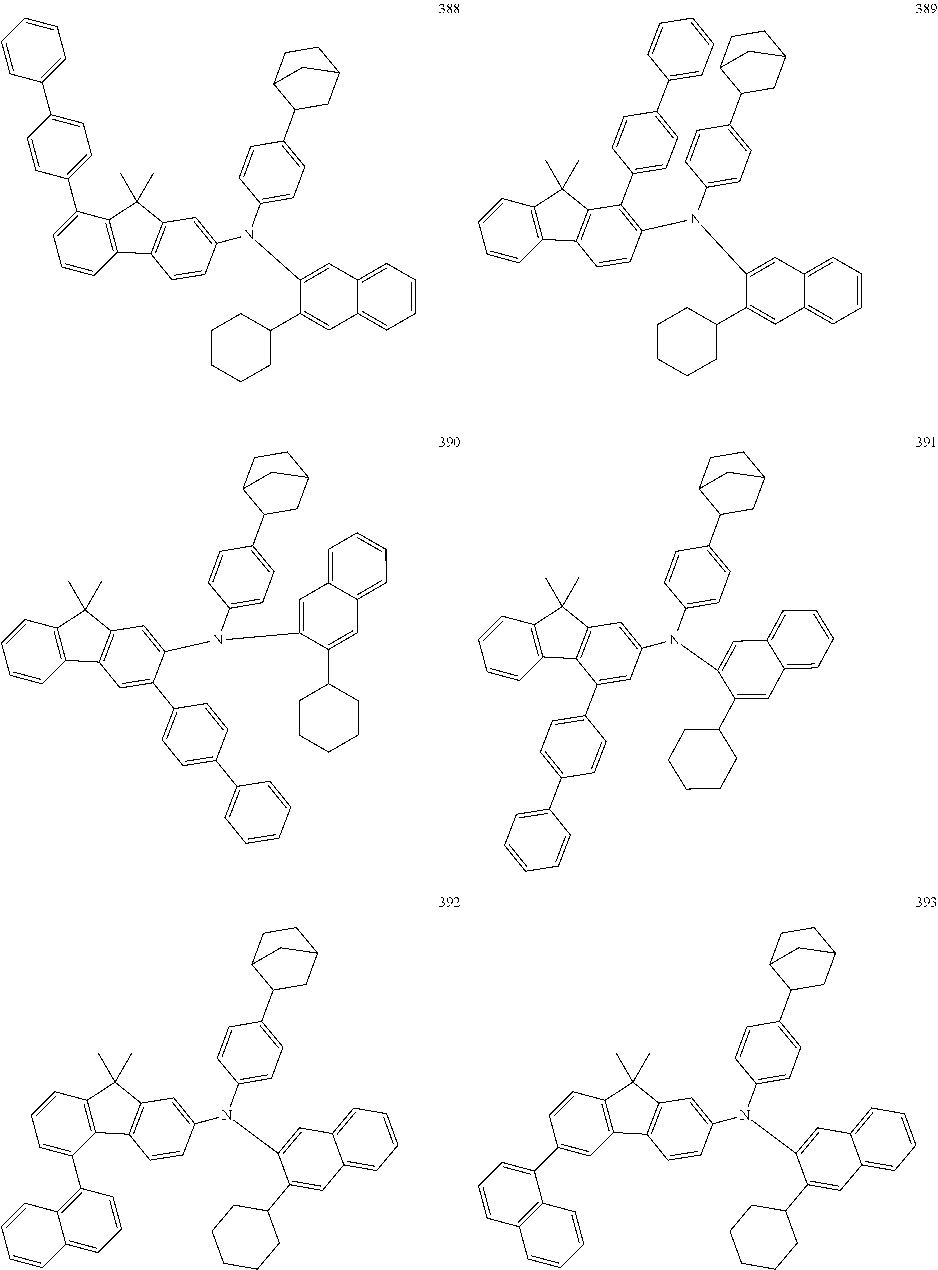 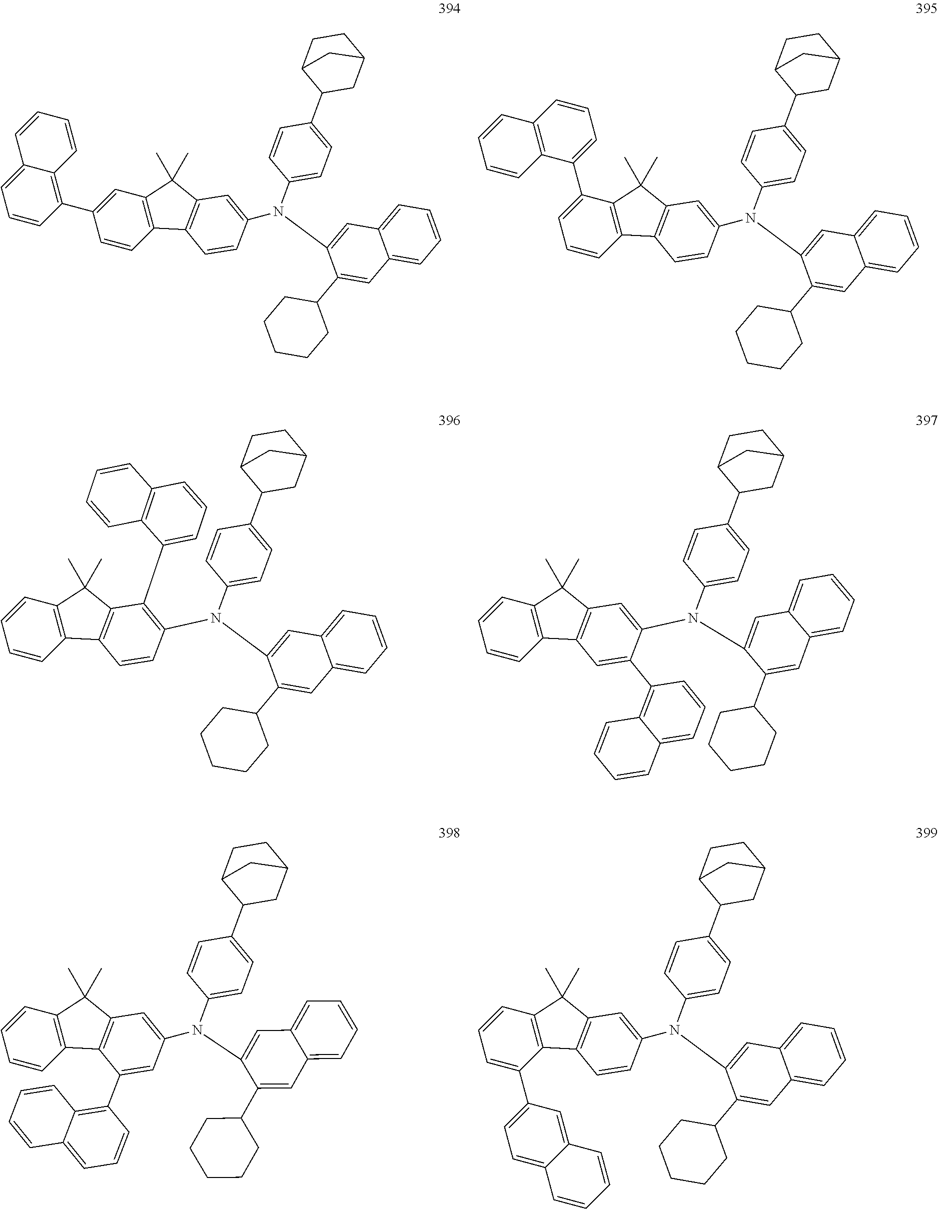 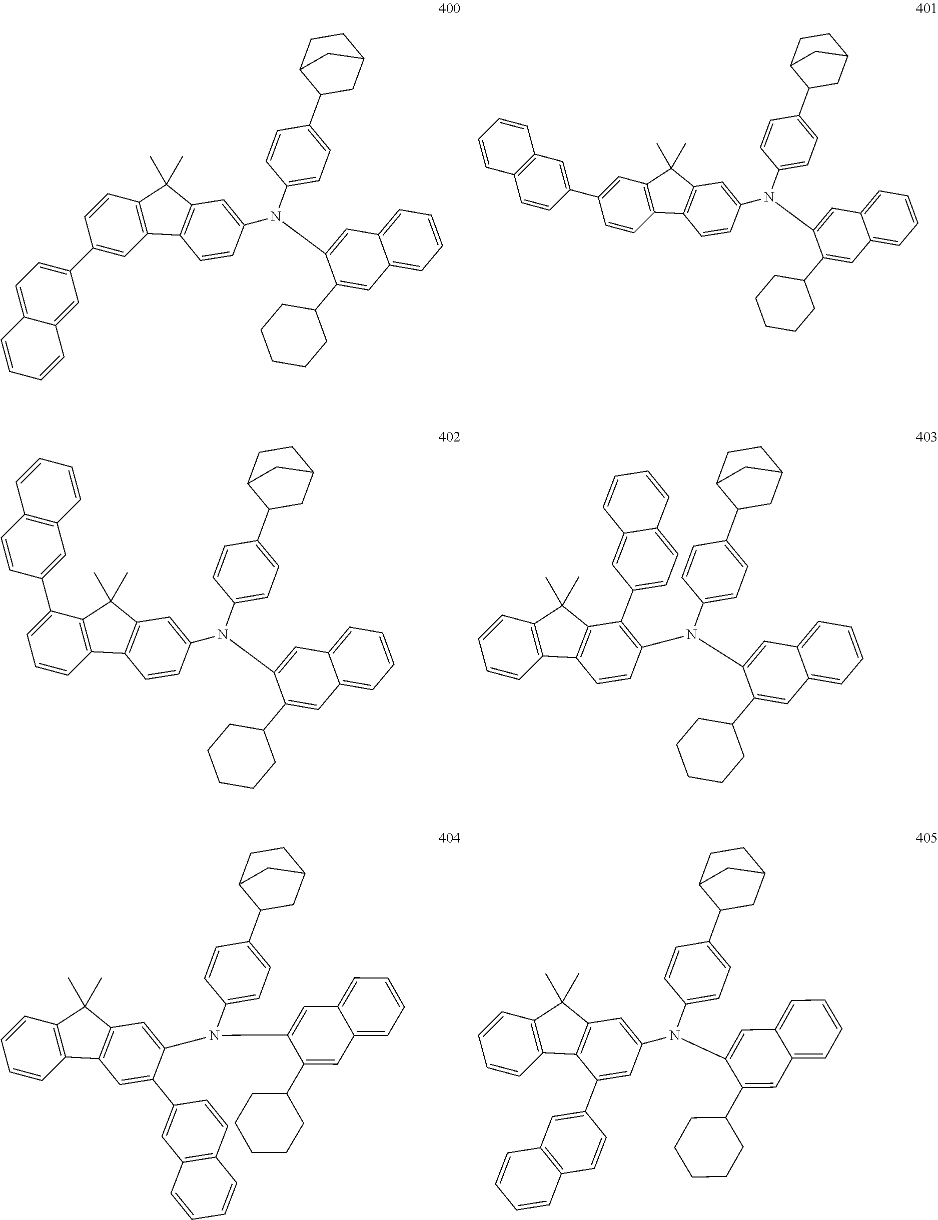 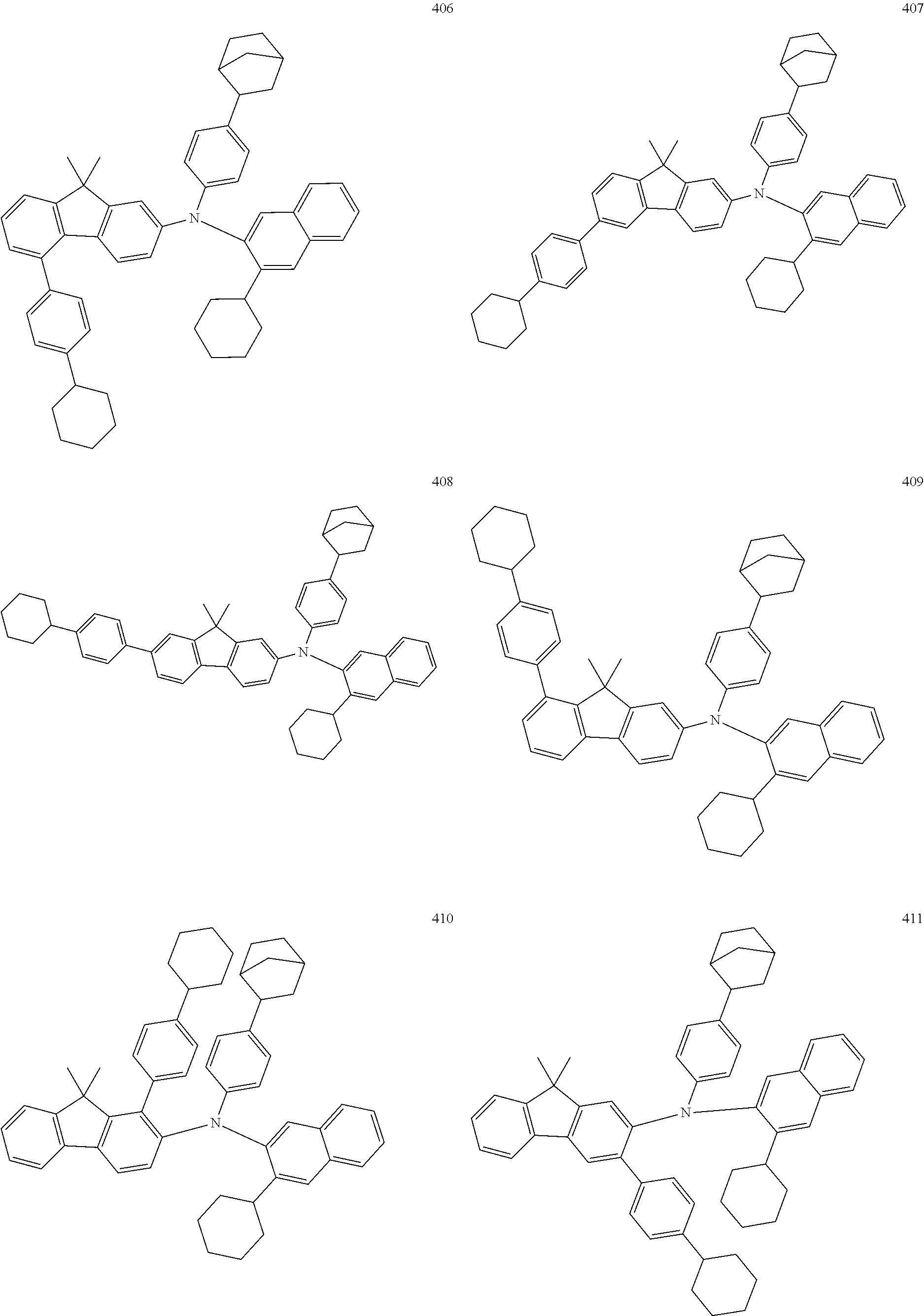 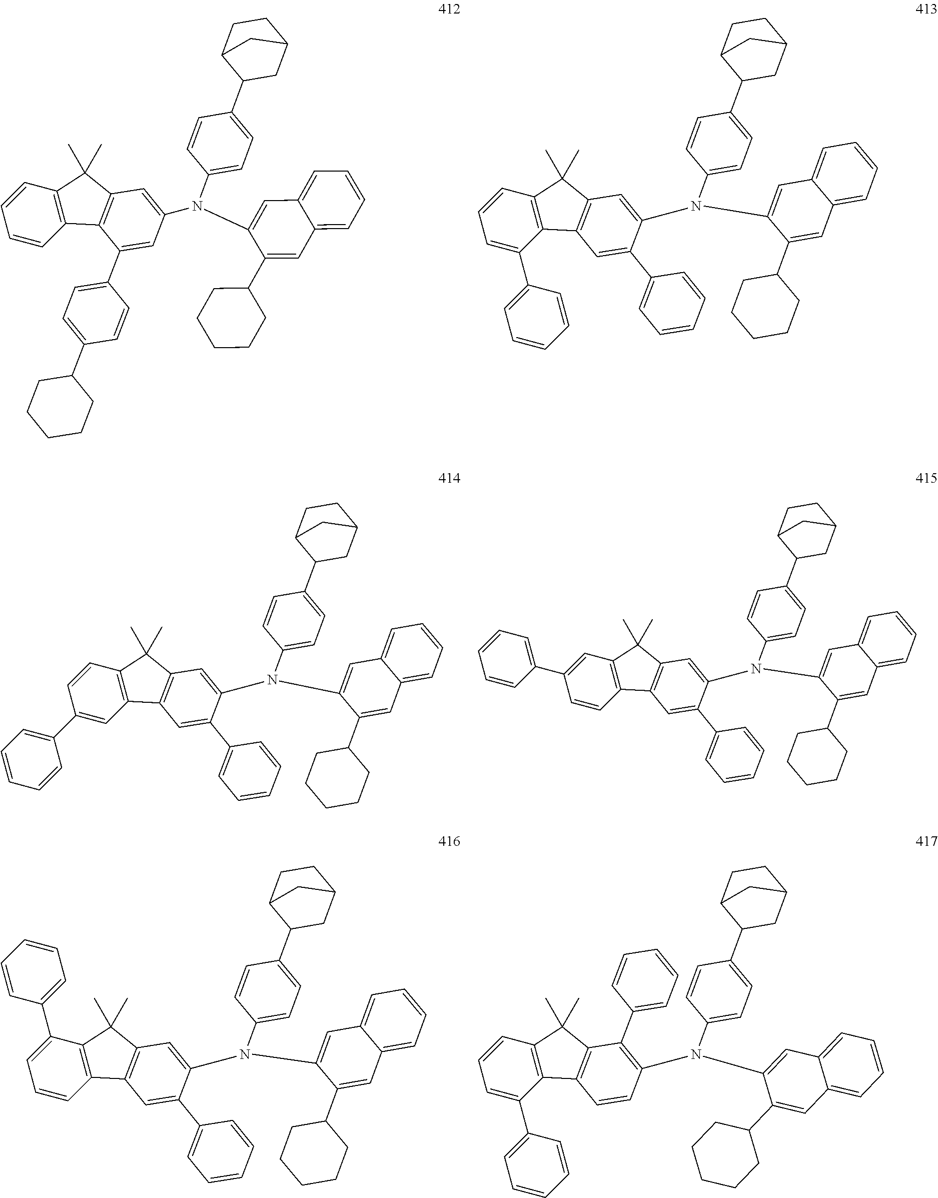 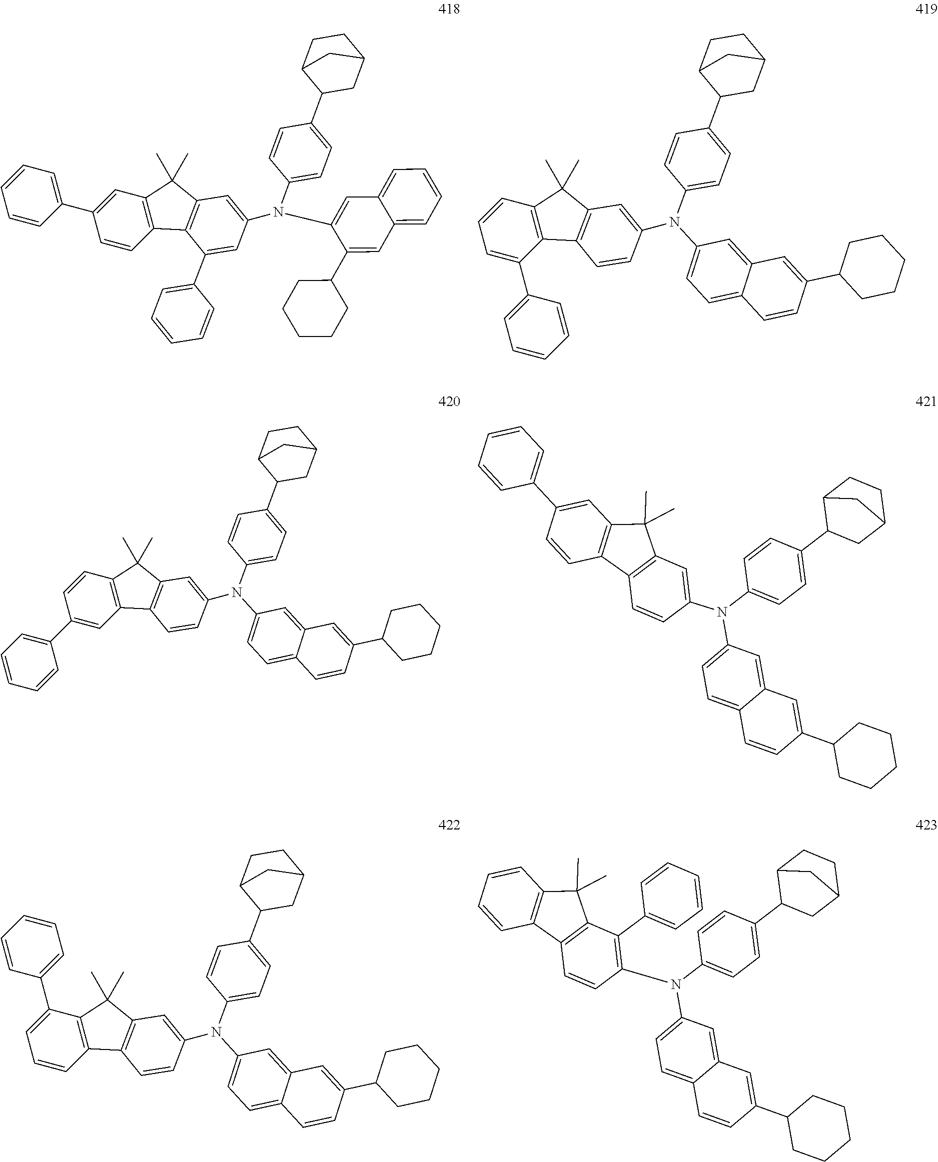 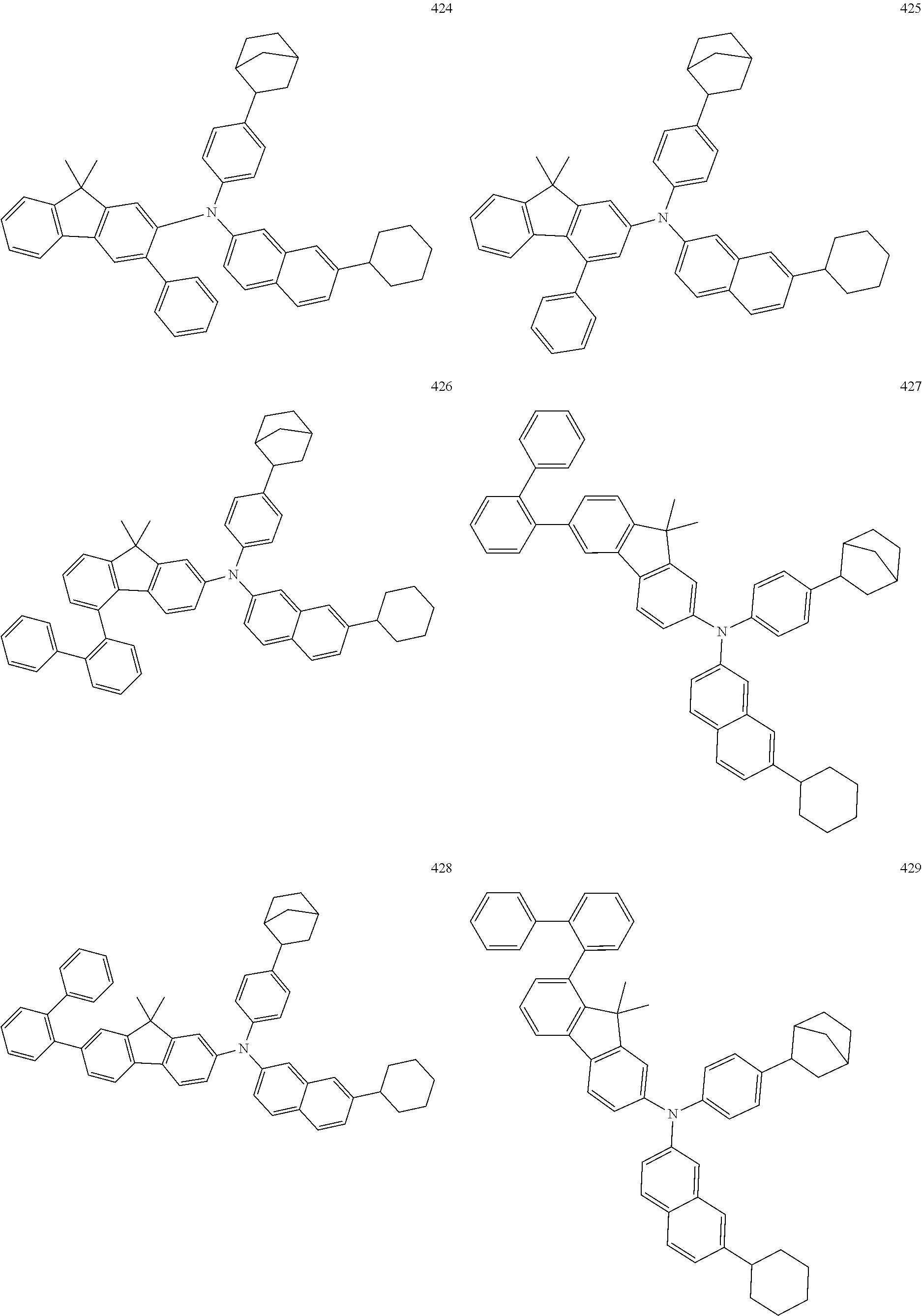 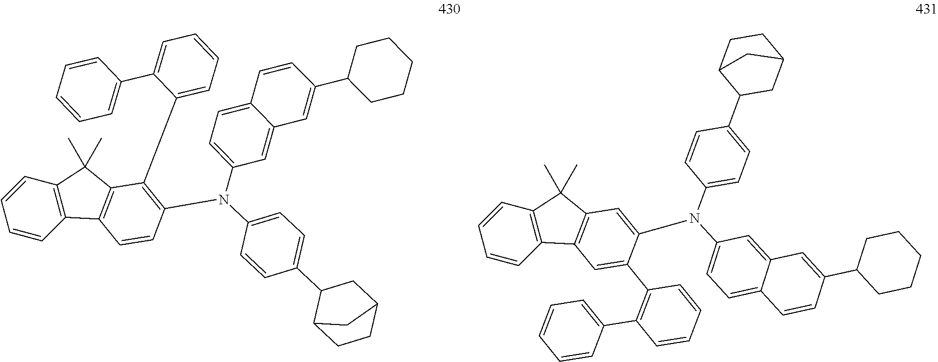 [SMILES: CC1(C)c2ccccc2-c2ccc(N(c3ccc(C4CC5CCC4C5)cc3)c3ccc4ccc(C5CCCCC5)cc4c3)c(-c3ccccc3-c3ccccc3)c21 ; CC1(C)c2ccccc2-c2cc(-c3ccccc3-c3ccccc3)c(N(c3ccc(C4CC5CCC4C5)cc3)c3ccc4ccc(C5CCCCC5)cc4c3)cc21] 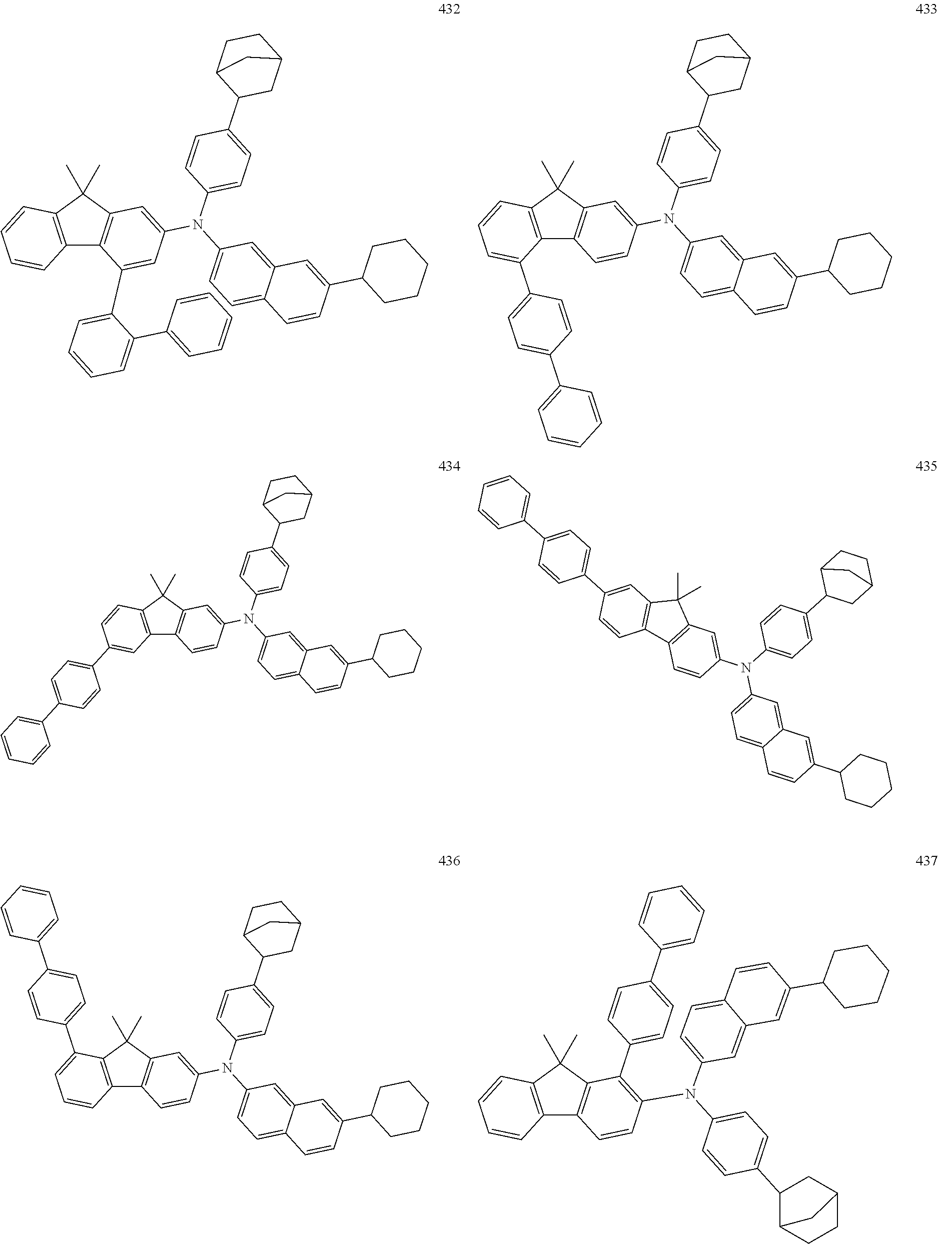 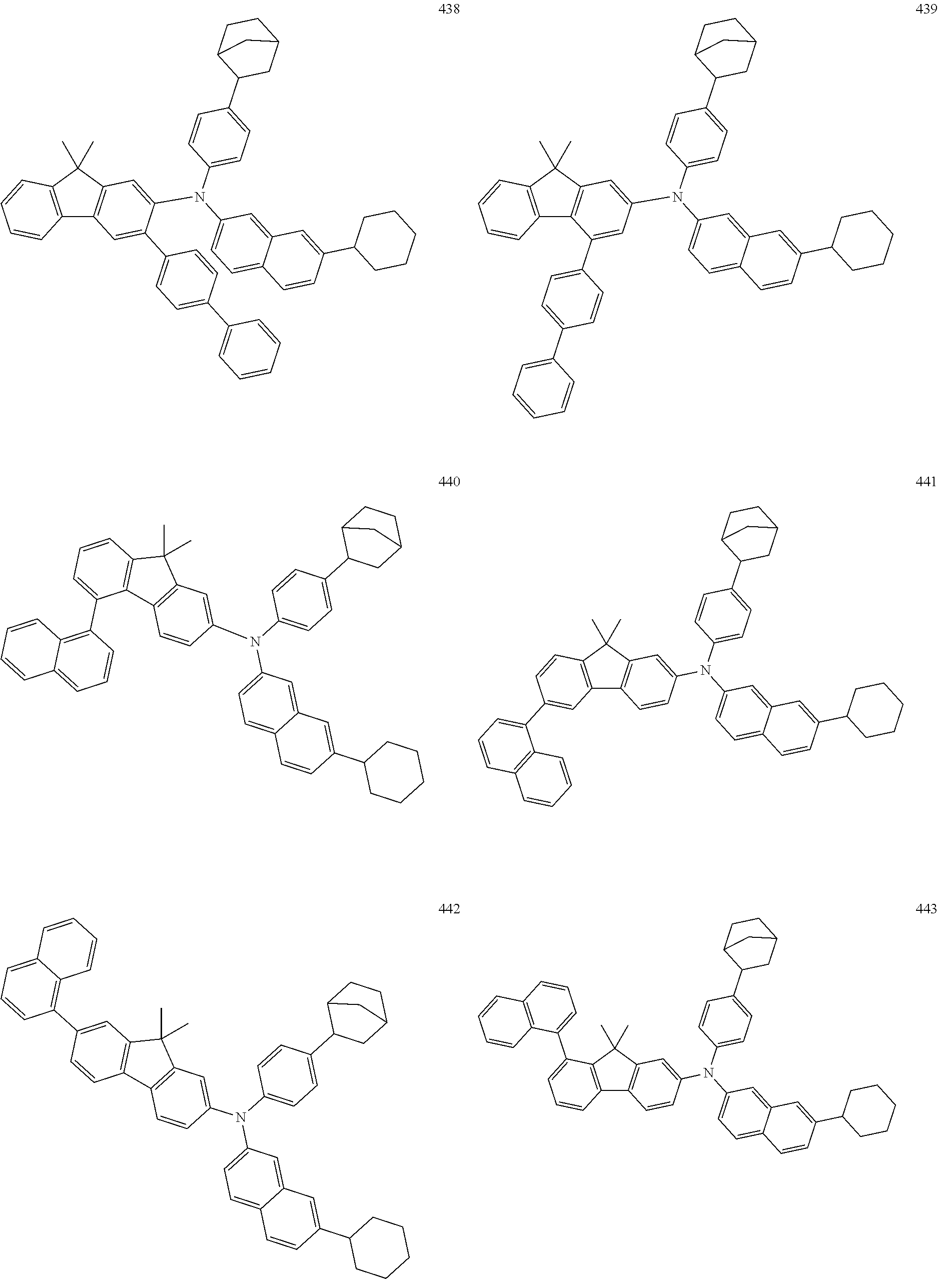 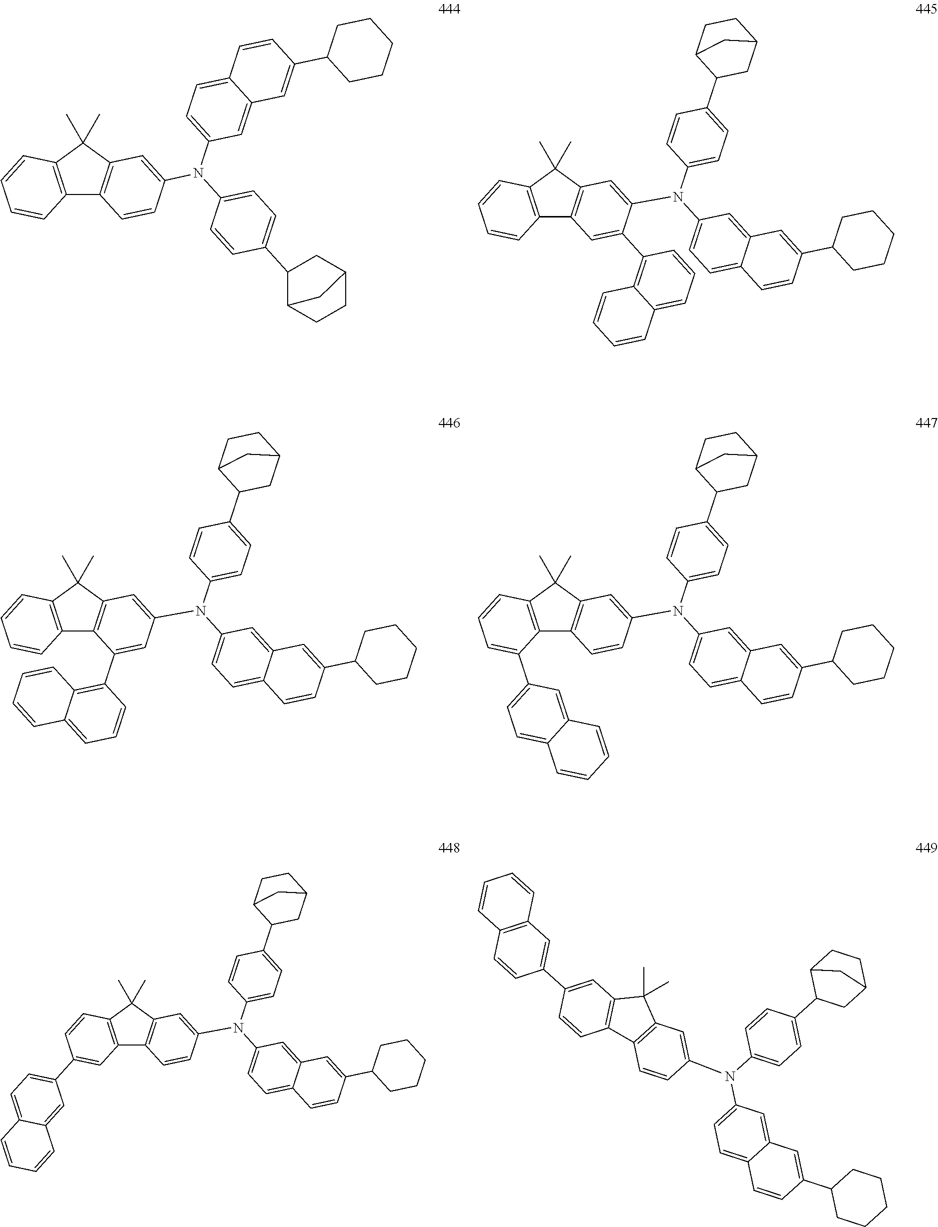 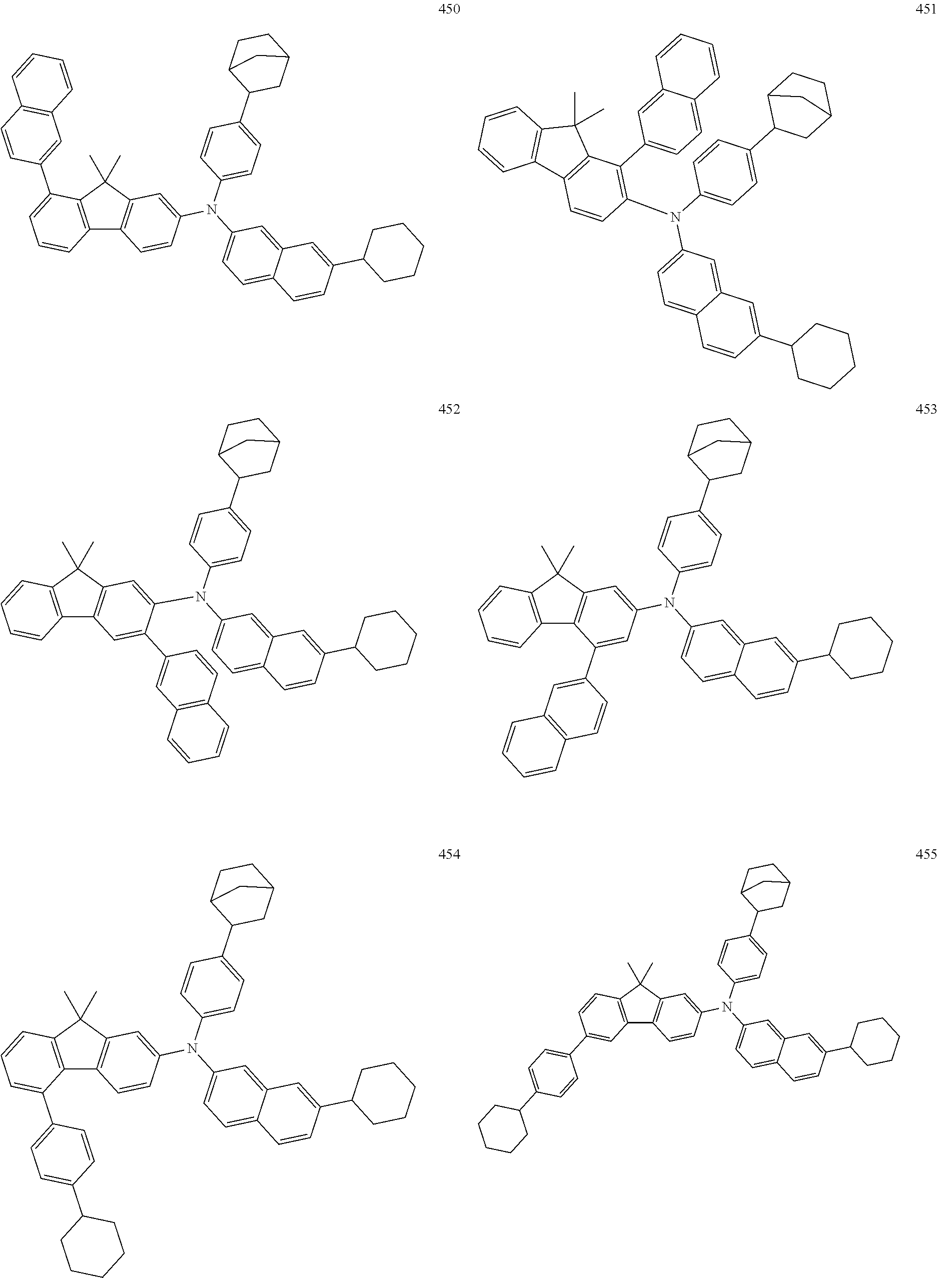 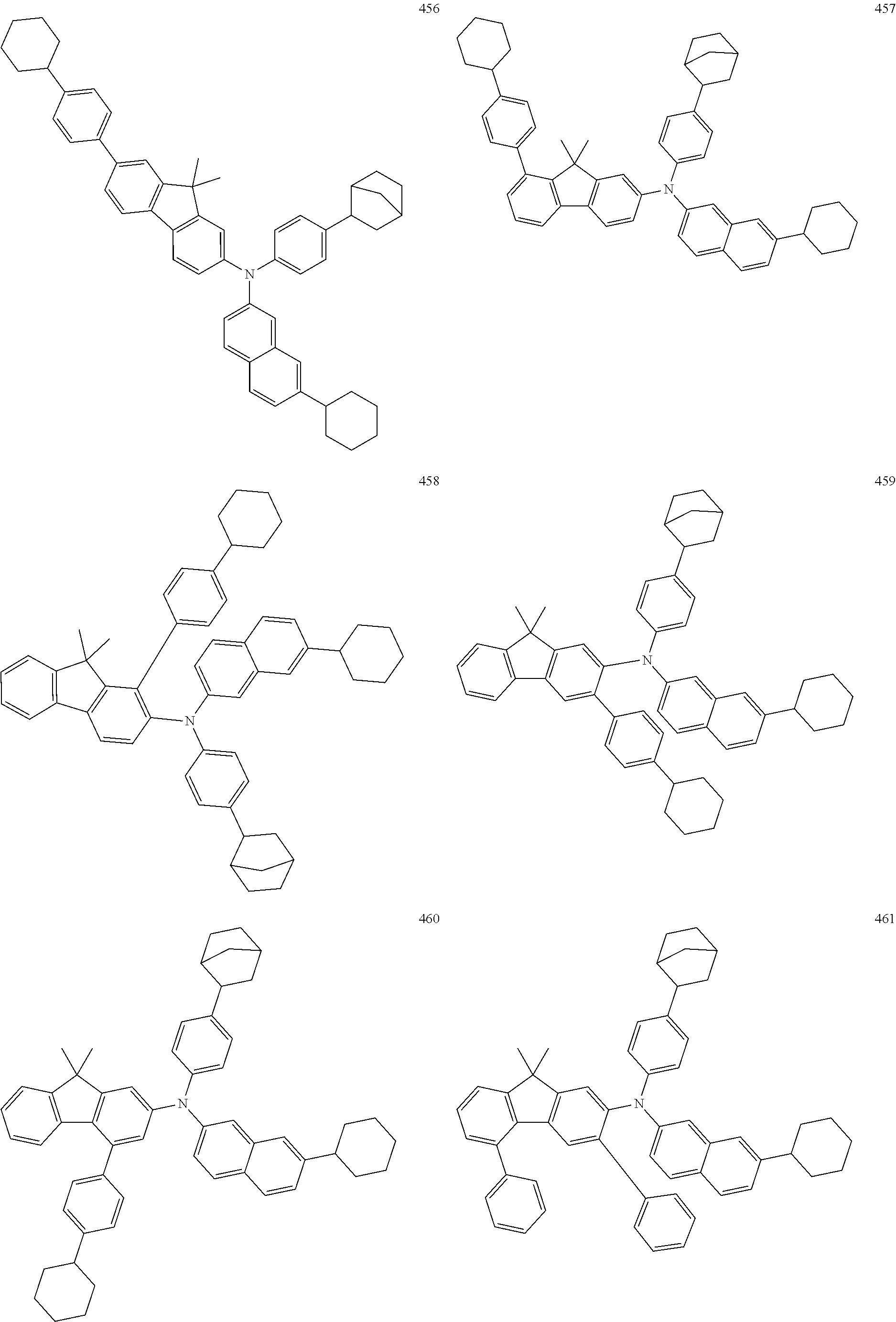 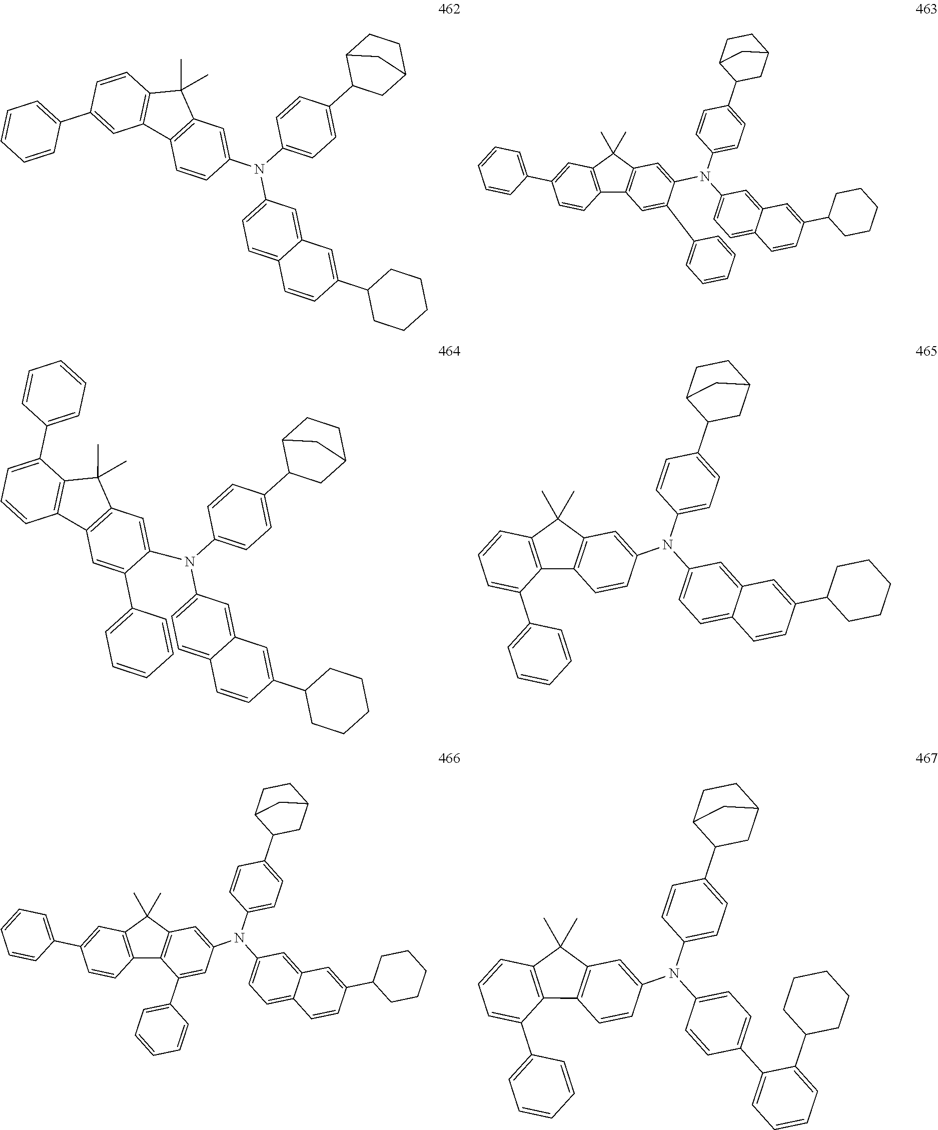 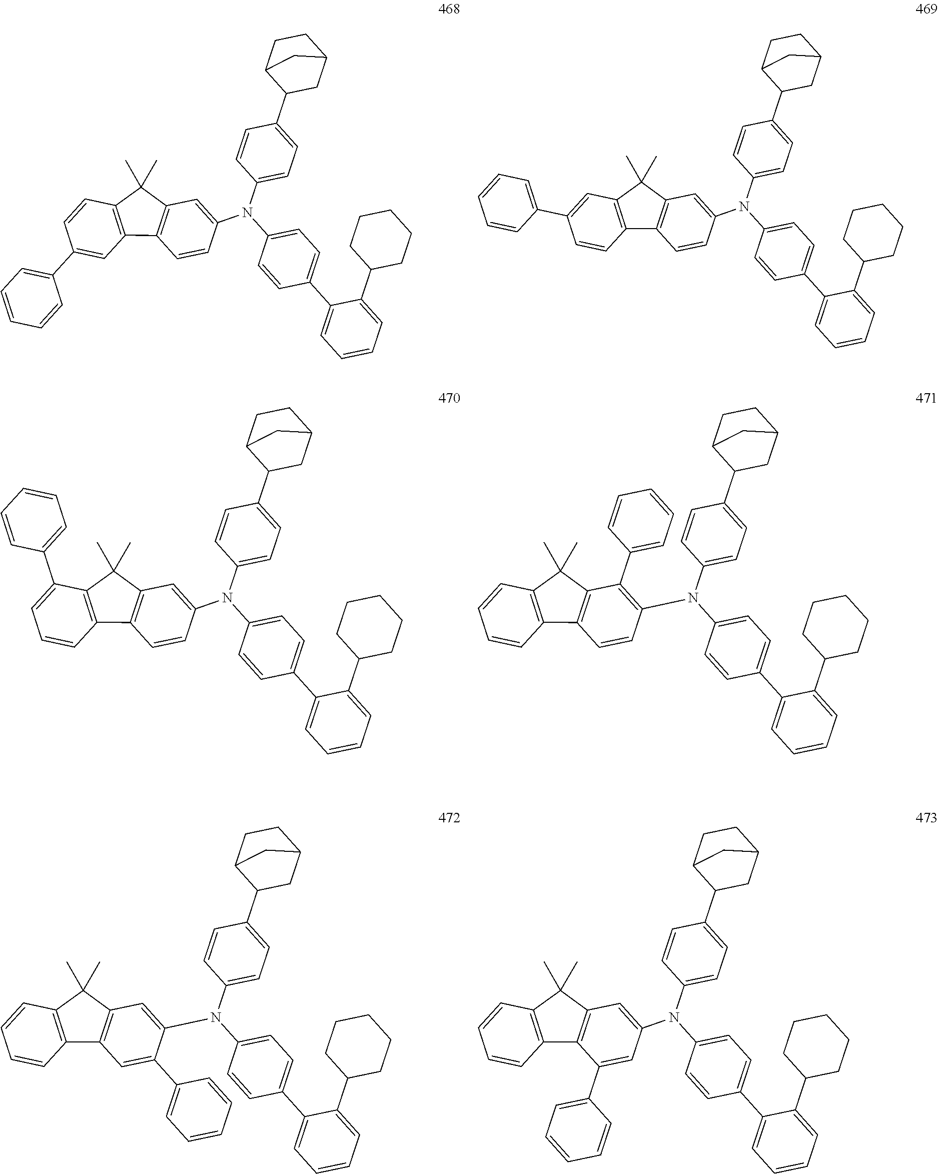 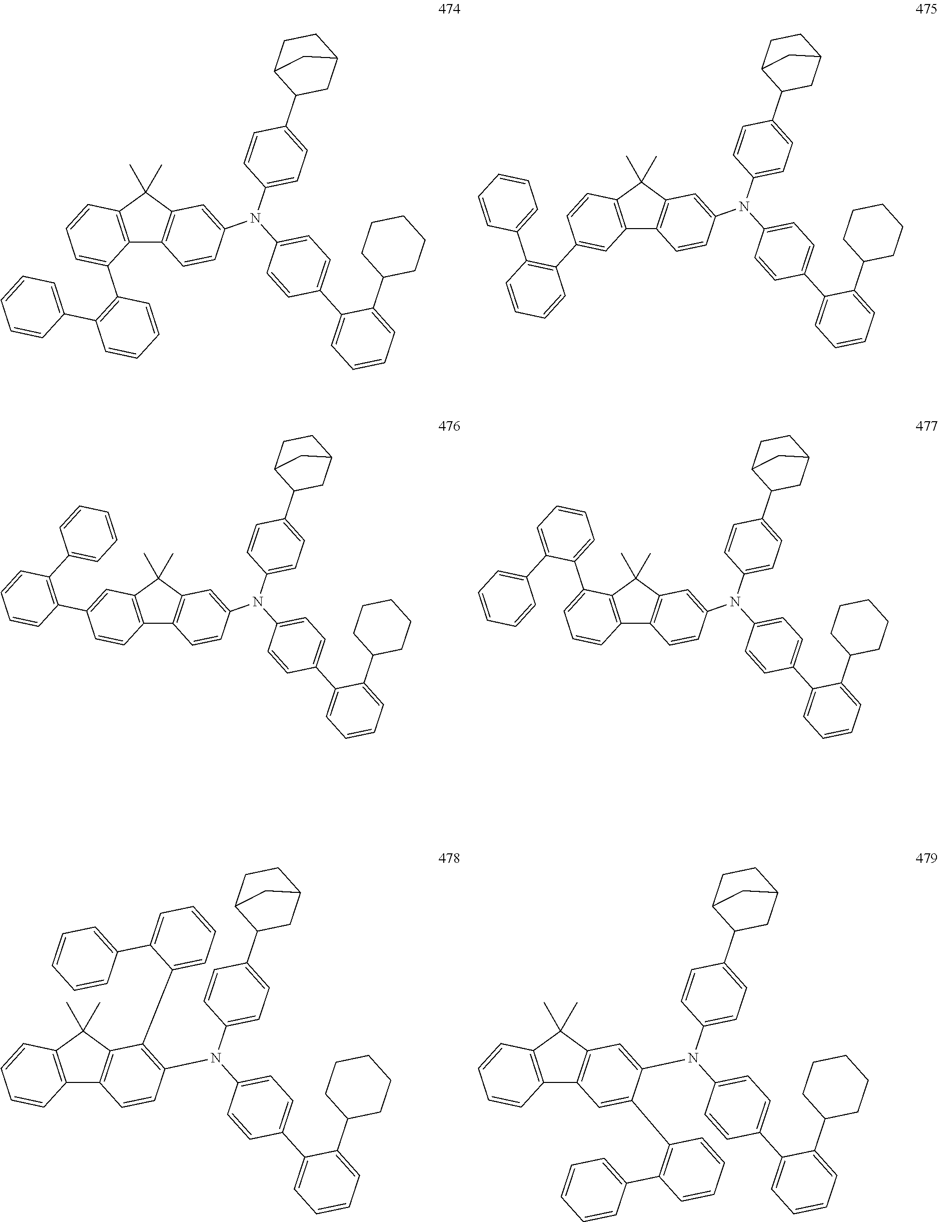 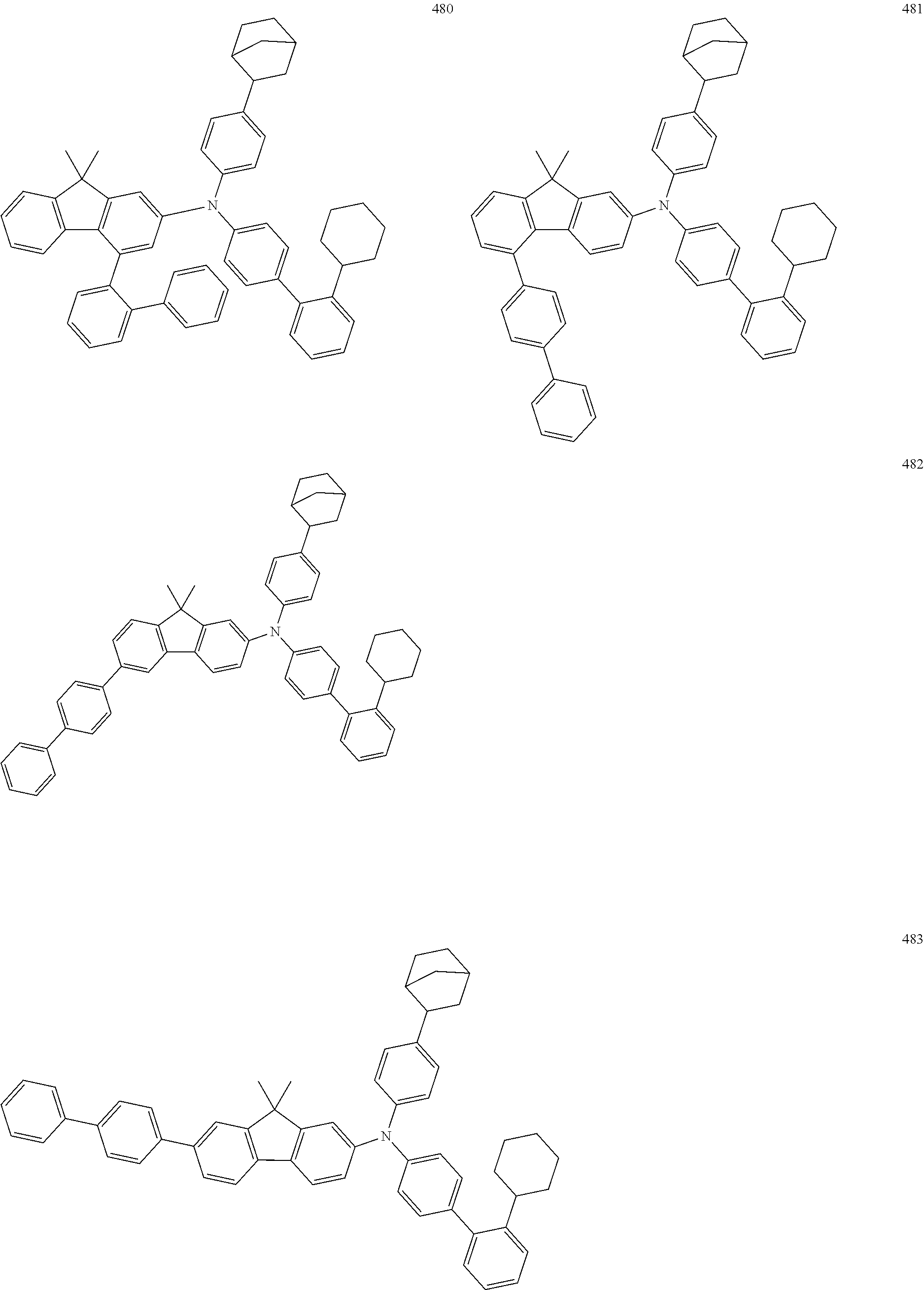 [SMILES: CC1(C)c2ccccc2-c2c(-c3ccccc3-c3ccccc3)cc(N(c3ccc(-c4ccccc4C4CCCCC4)cc3)c3ccc(C4CC5CCC4C5)cc3)cc21 ; CC1(C)c2cc(N(c3ccc(-c4ccccc4C4CCCCC4)cc3)c3ccc(C4CC5CCC4C5)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(-c4ccccc4C4CCCCC4)cc3)c3ccc(C4CC5CCC4C5)cc3)cc21 ; CC1(C)c2cc(-c3ccc(-c4ccccc4)cc3)ccc2-c2ccc(N(c3ccc(-c4ccccc4C4CCCCC4)cc3)c3ccc(C4CC5CCC4C5)cc3)cc21] 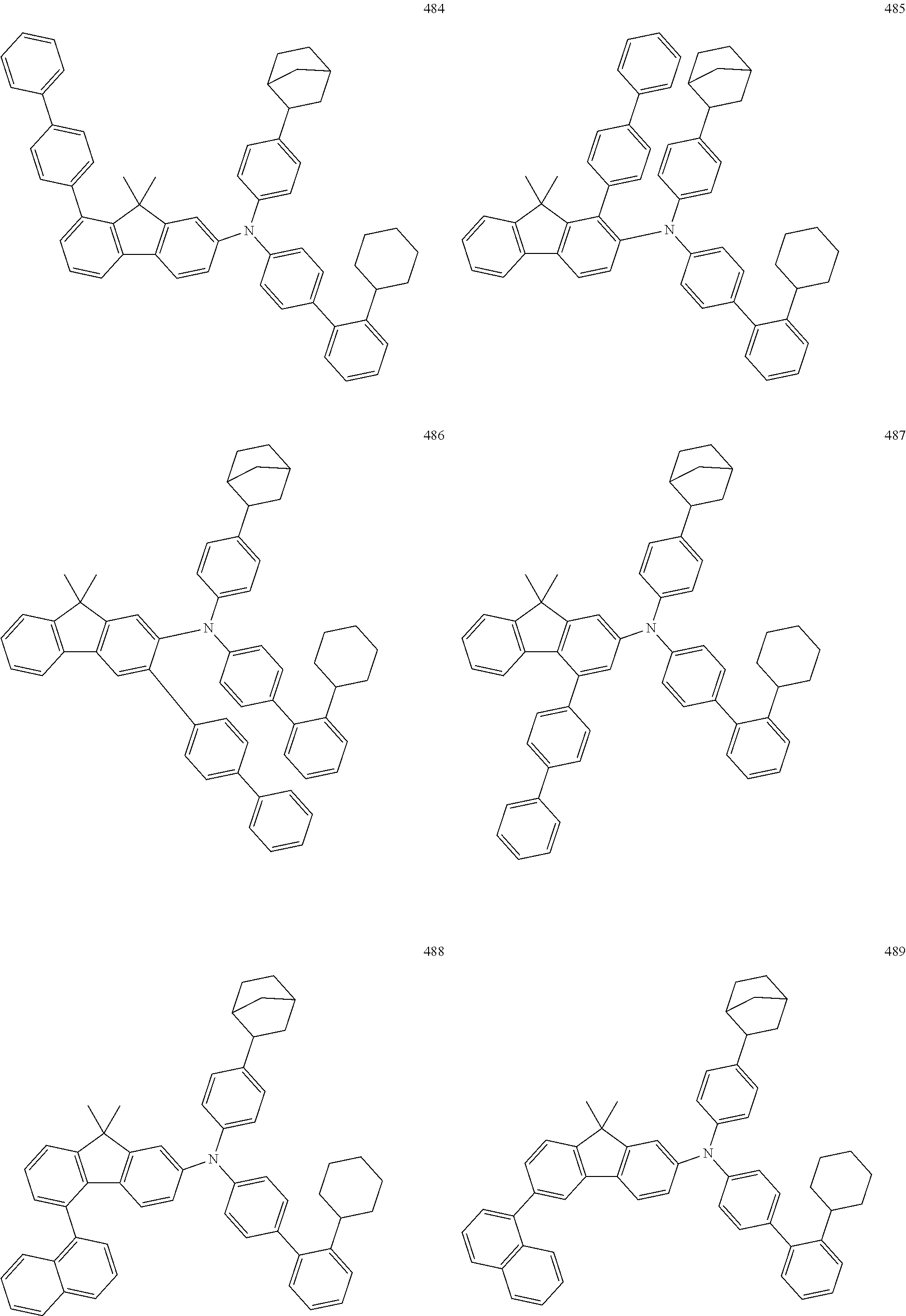 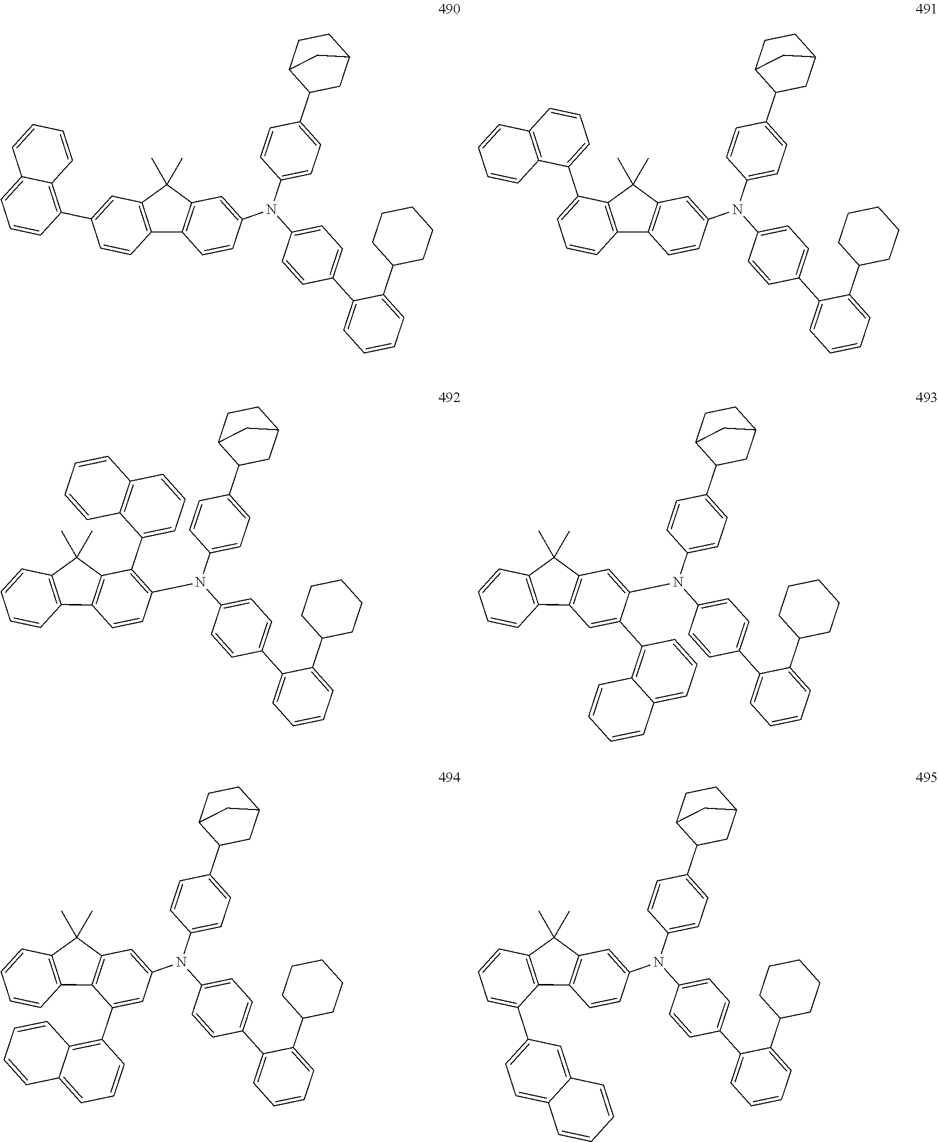 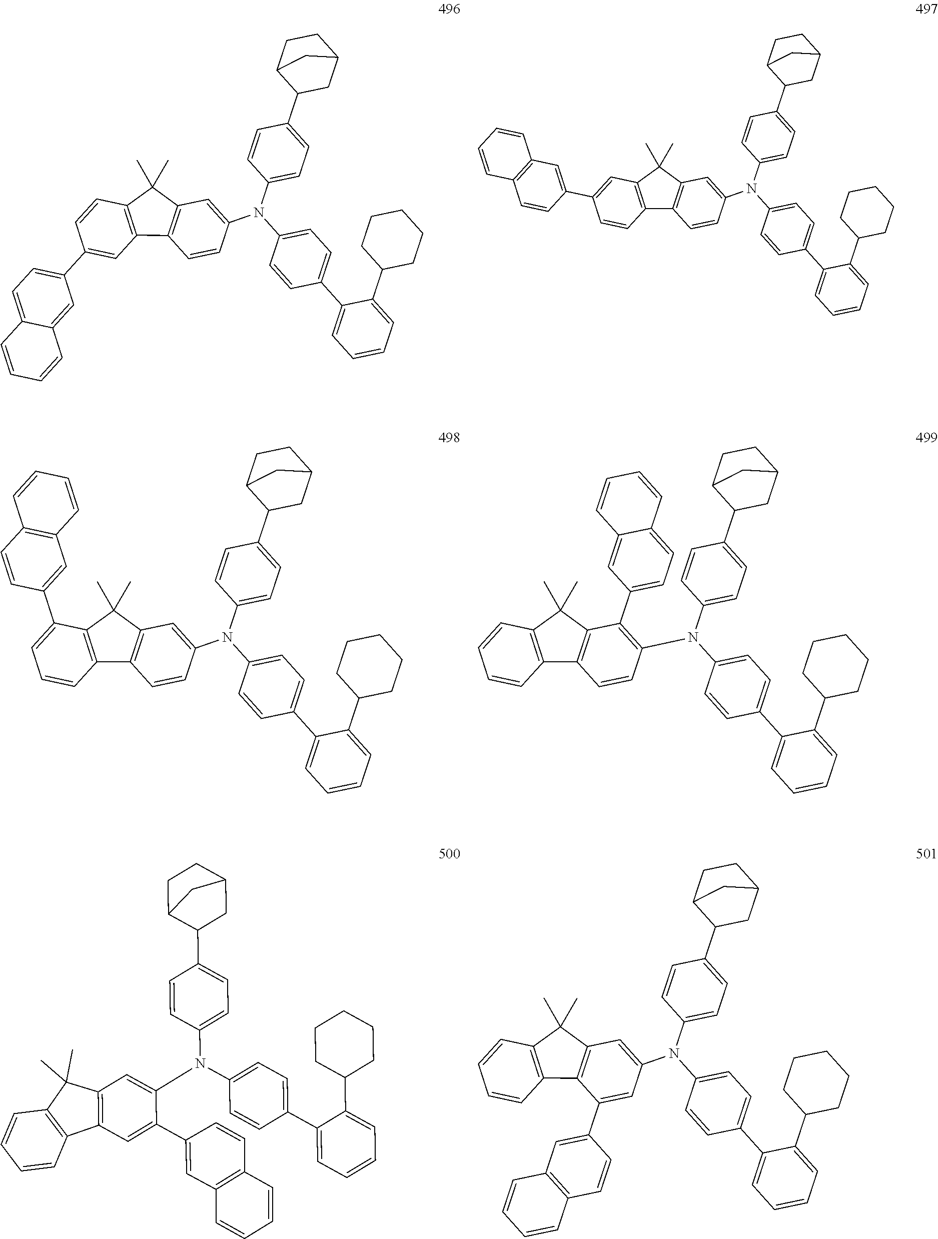 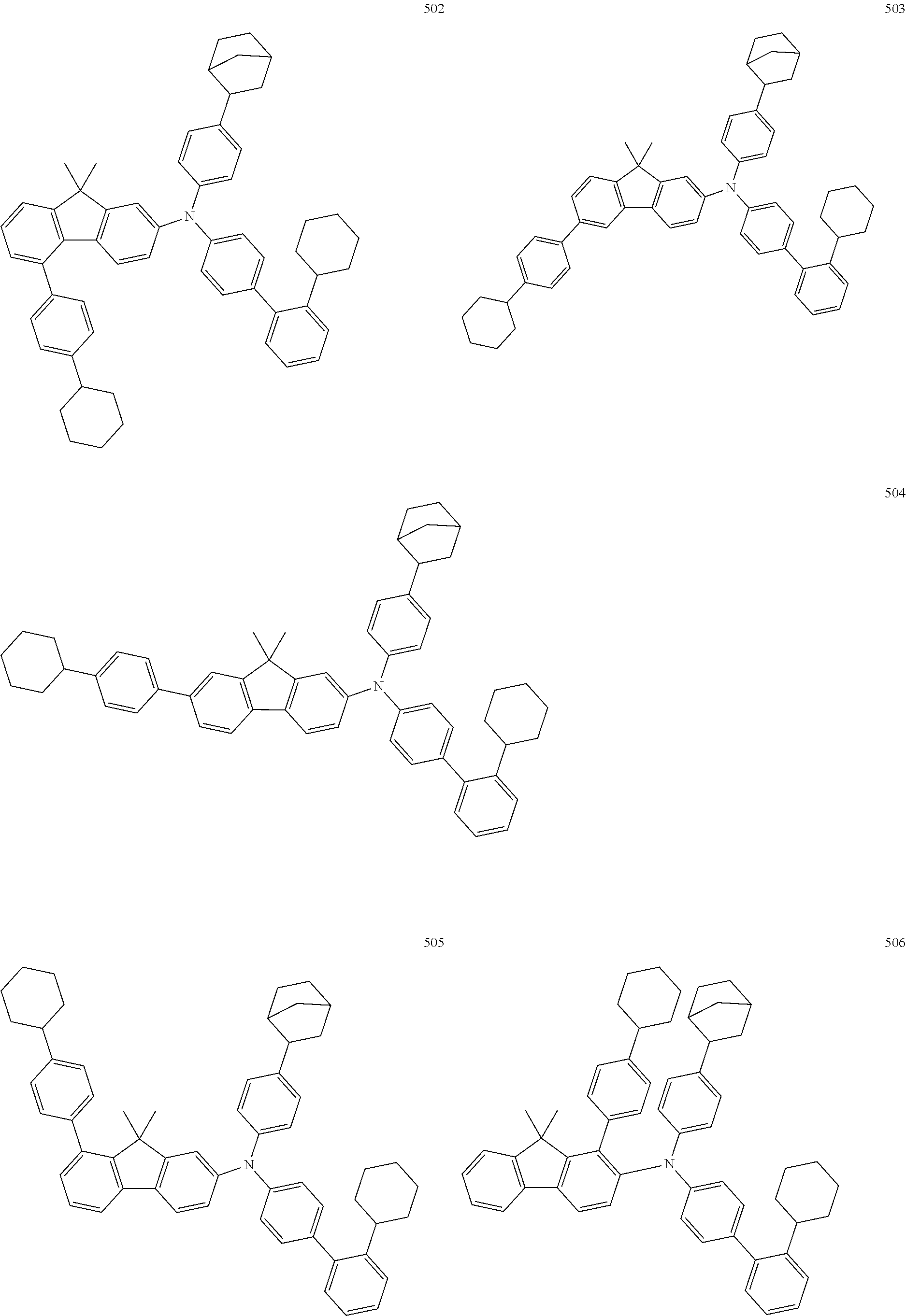 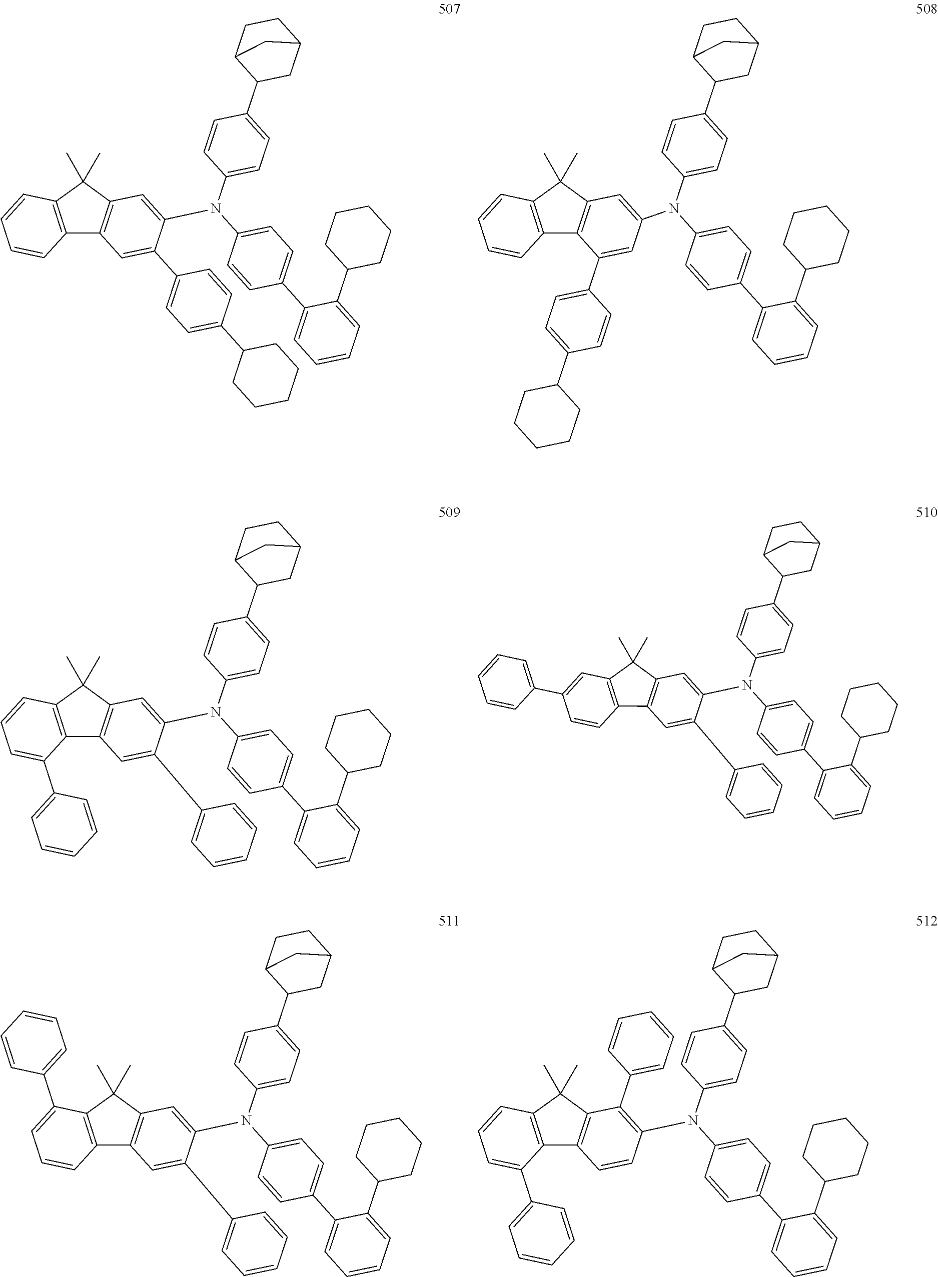 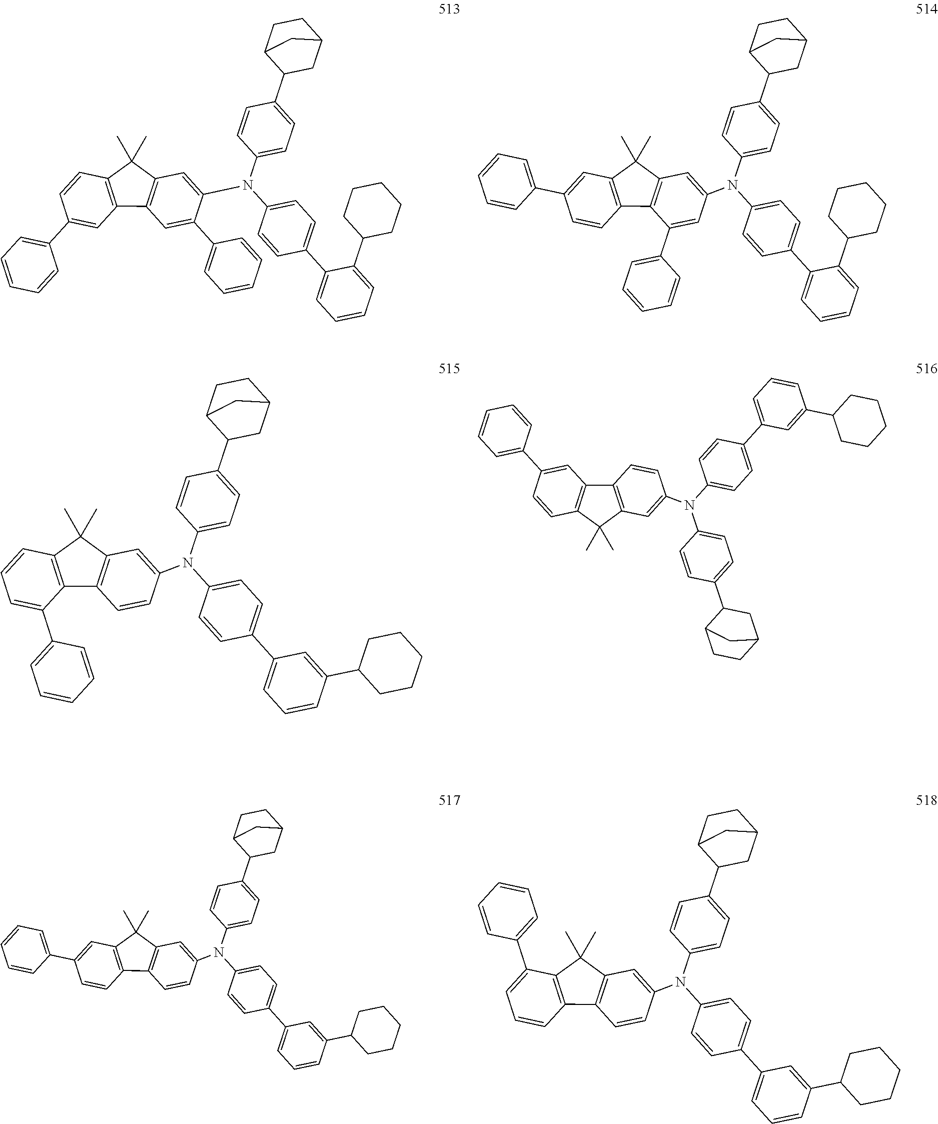 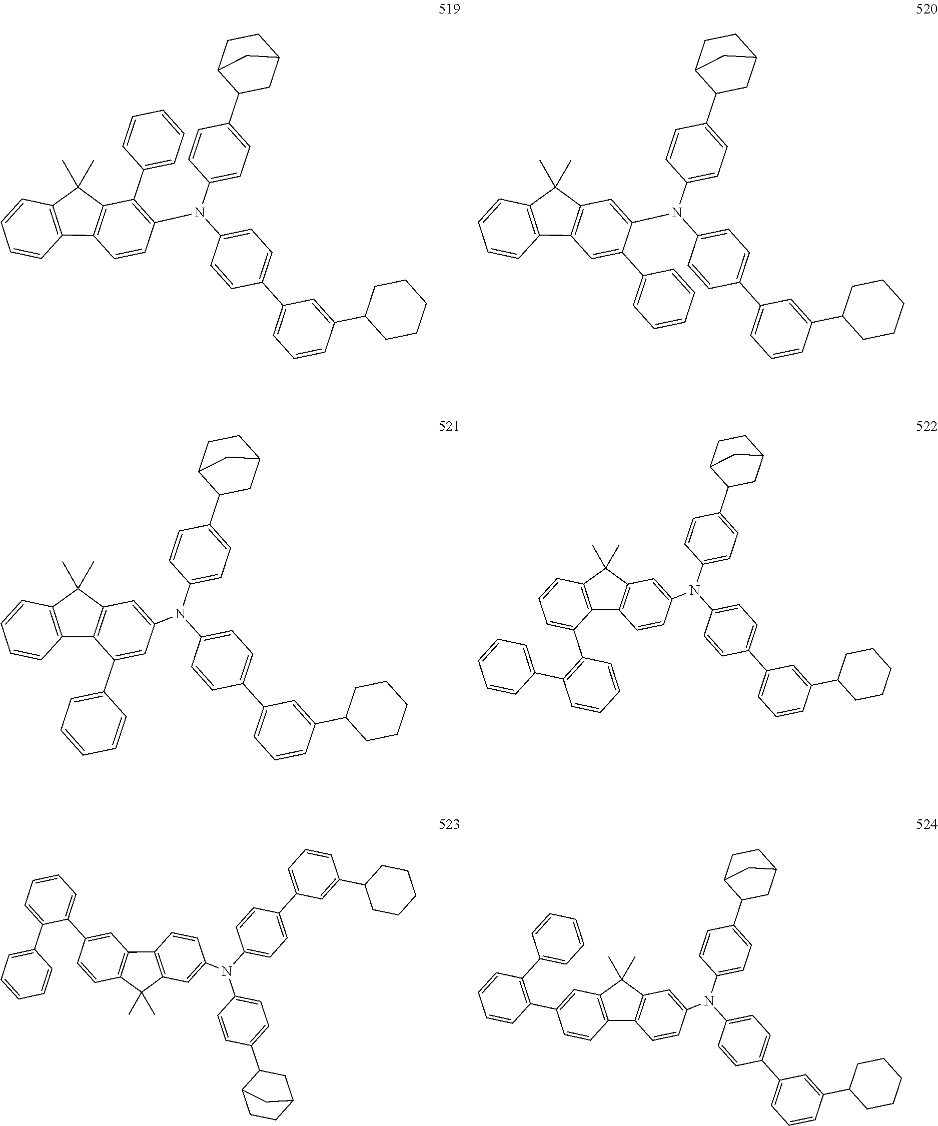 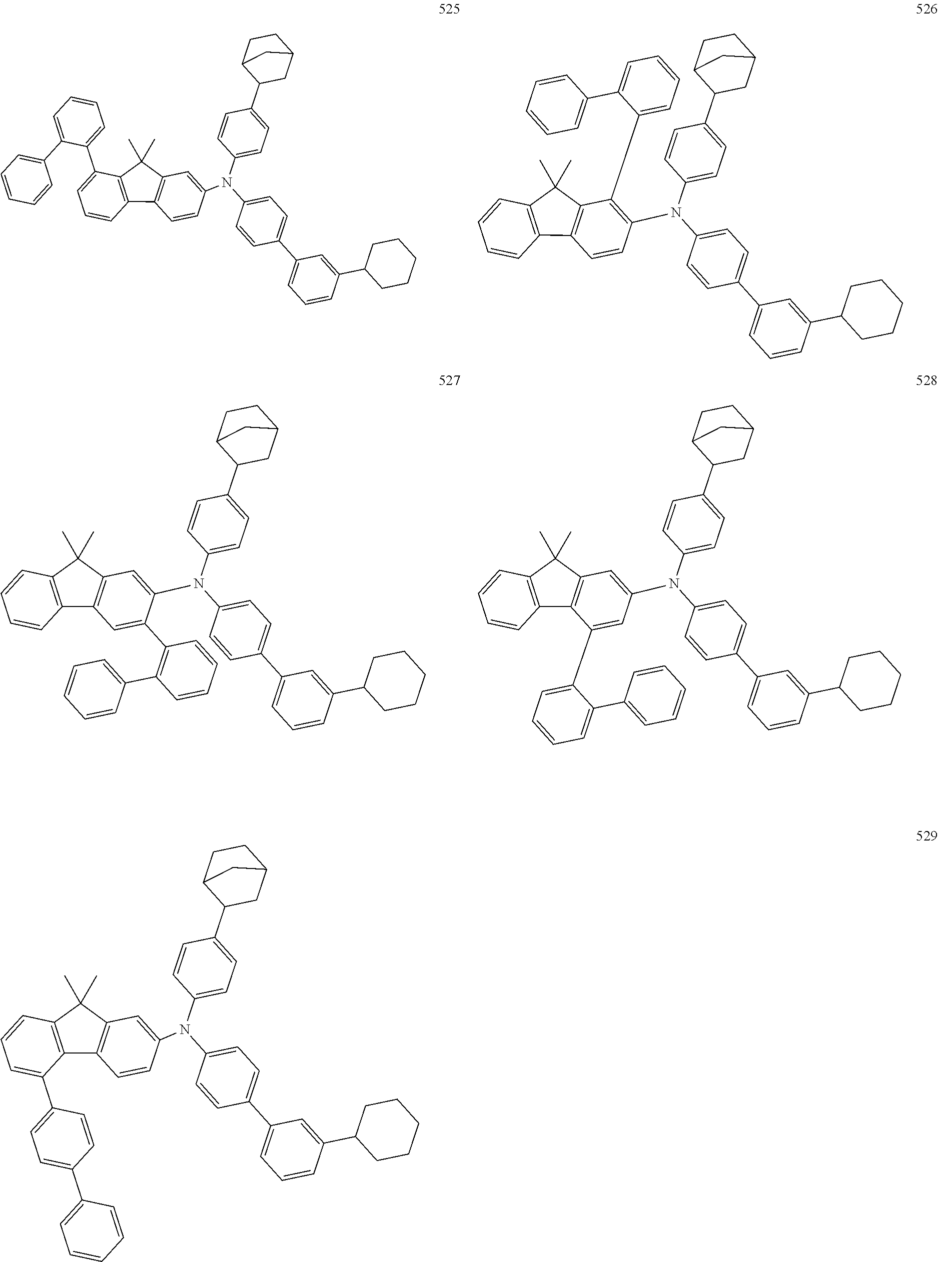 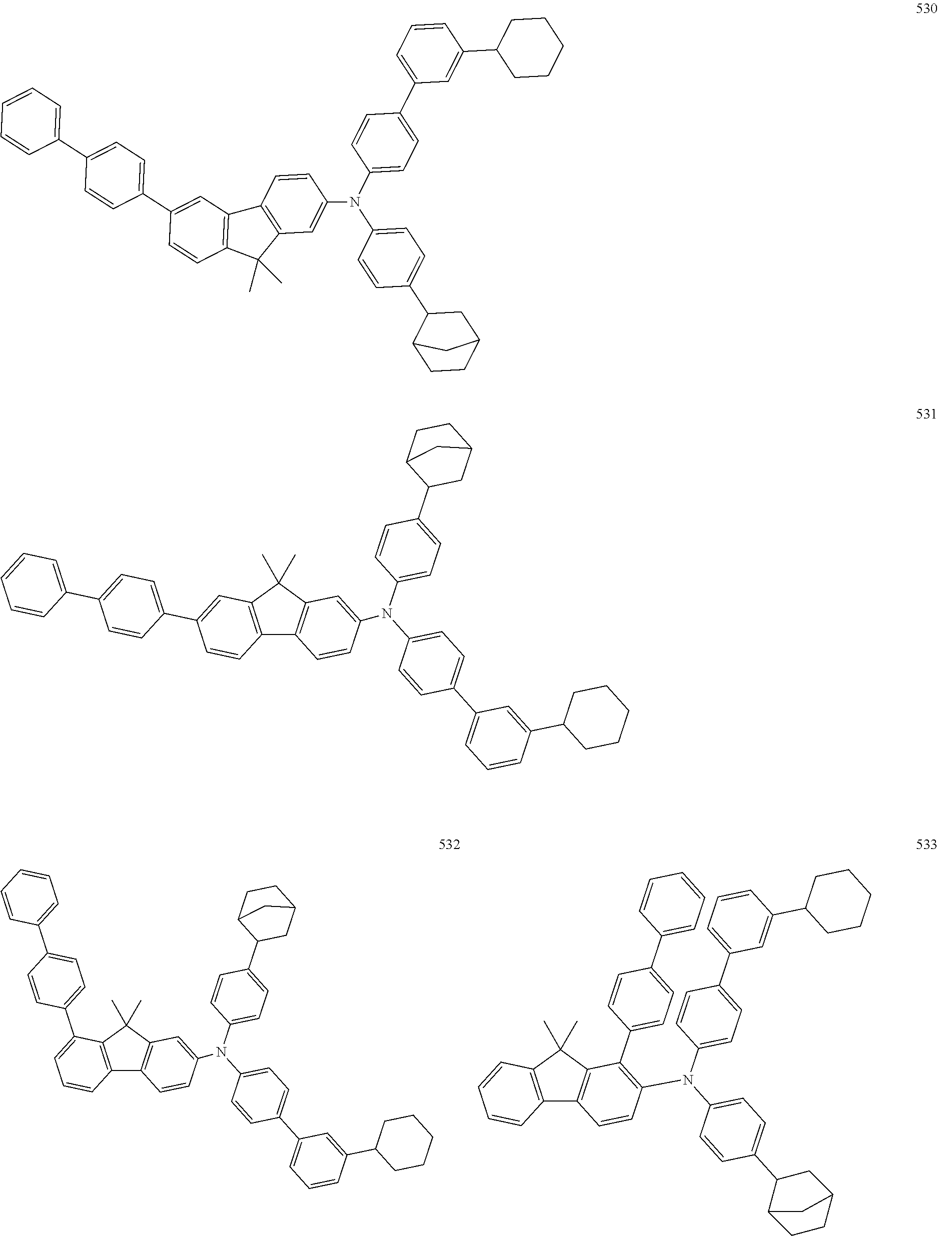 [SMILES: CC1(C)c2ccc(-c3ccc(-c4ccccc4)cc3)cc2-c2ccc(N(c3ccc(-c4cccc(C5CCCCC5)c4)cc3)c3ccc(C4CC5CCC4C5)cc3)cc21 ; CC1(C)c2cc(-c3ccc(-c4ccccc4)cc3)ccc2-c2ccc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2cccc(-c3ccc(-c4ccccc4)cc3)c21 ; CC1(C)c2ccccc2-c2ccc(N(c3ccc(-c4cccc(C5CCCCC5)c4)cc3)c3ccc(C4CC5CCC4C5)cc3)c(-c3ccc(-c4ccccc4)cc3)c21] 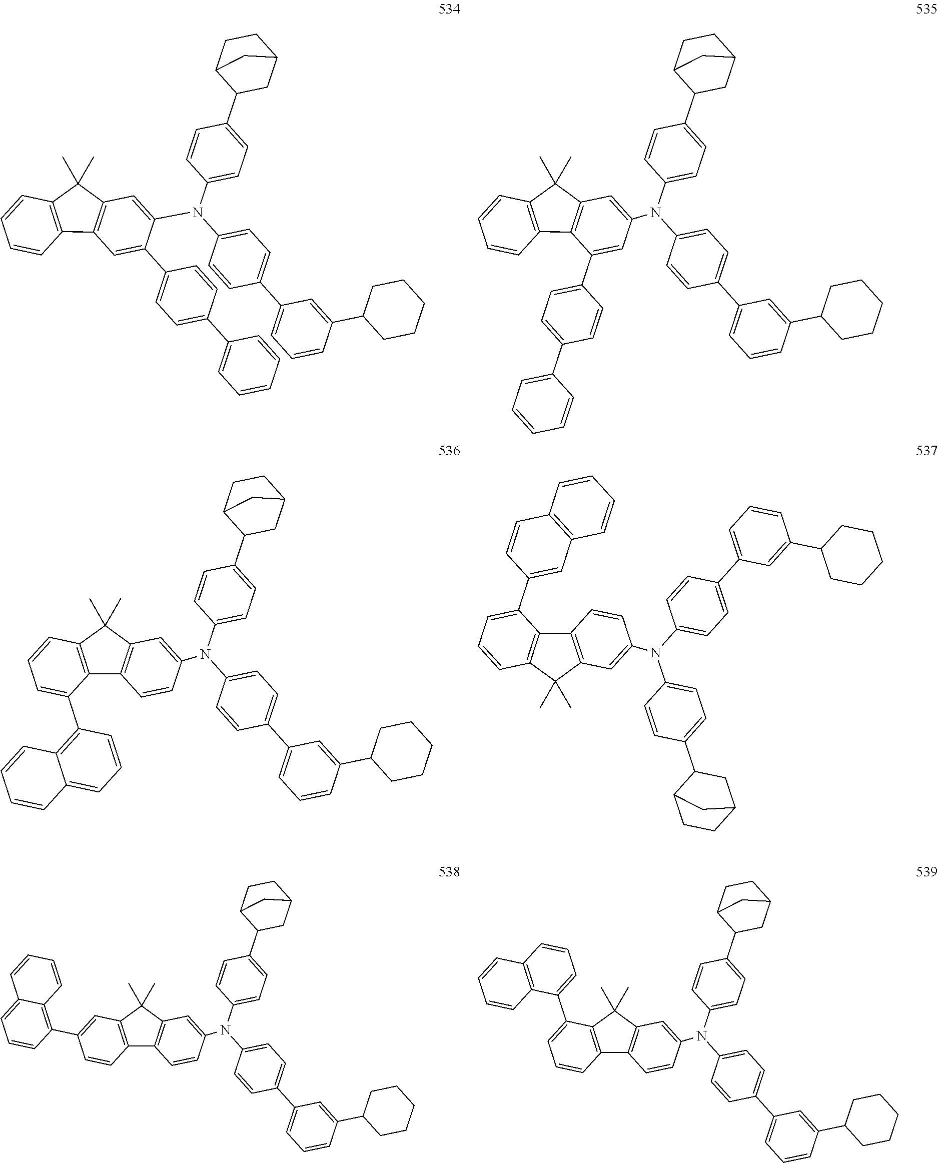 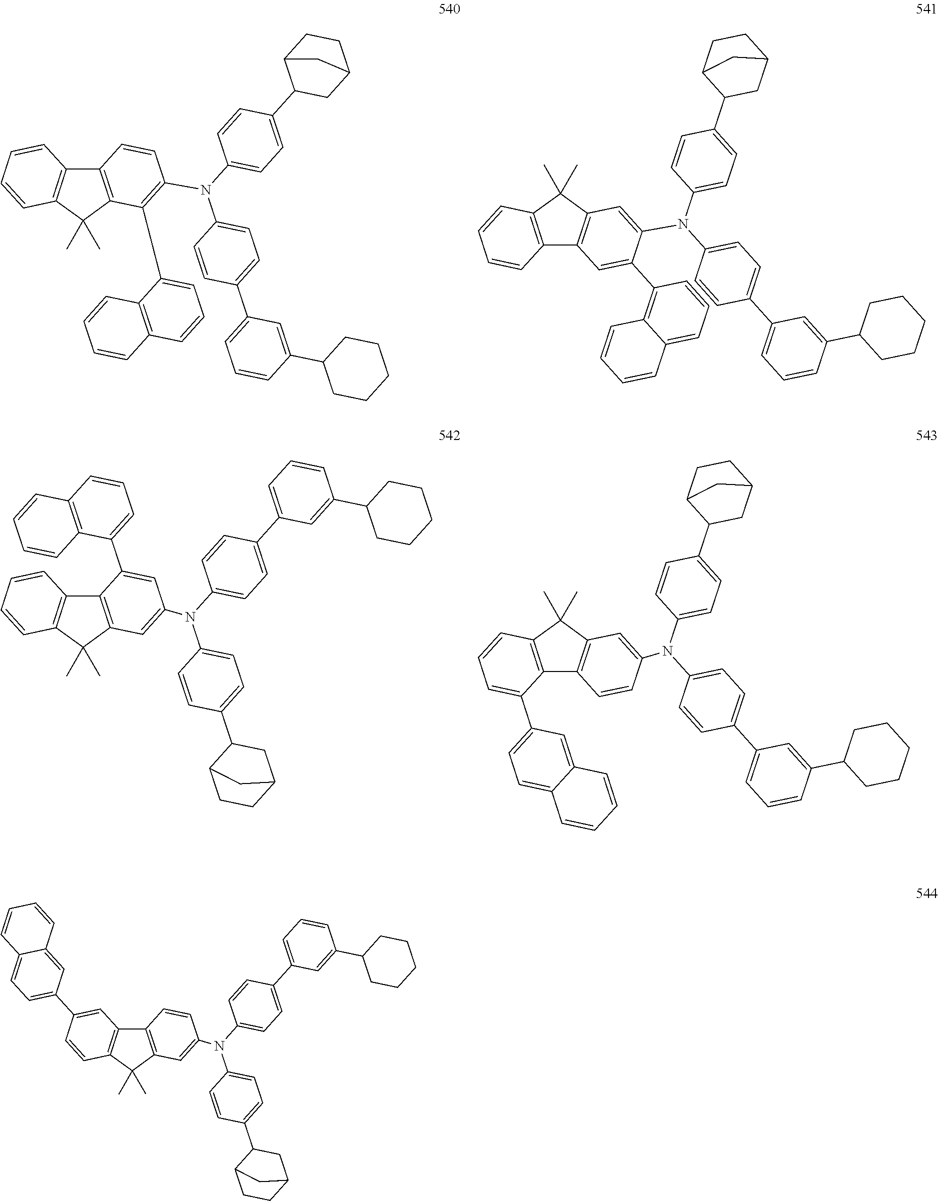 [SMILES: CC1(C)c2ccccc2-c2ccc(N(c3ccc(-c4cccc(C5CCCCC5)c4)cc3)c3ccc(C4CC5CCC4C5)cc3)c(-c3cccc4ccccc34)c21 ; CC1(C)c2ccccc2-c2cc(-c3cccc4ccccc34)c(N(c3ccc(-c4cccc(C5CCCCC5)c4)cc3)c3ccc(C4CC5CCC4C5)cc3)cc21 ; CC1(C)c2ccccc2-c2c(-c3cccc4ccccc34)cc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2c(-c3ccc4ccccc4c3)cccc21 ; CC1(C)c2ccc(-c3ccc4ccccc4c3)cc2-c2ccc(N(c3ccc(-c4cccc(C5CCCCC5)c4)cc3)c3ccc(C4CC5CCC4C5)cc3)cc21] 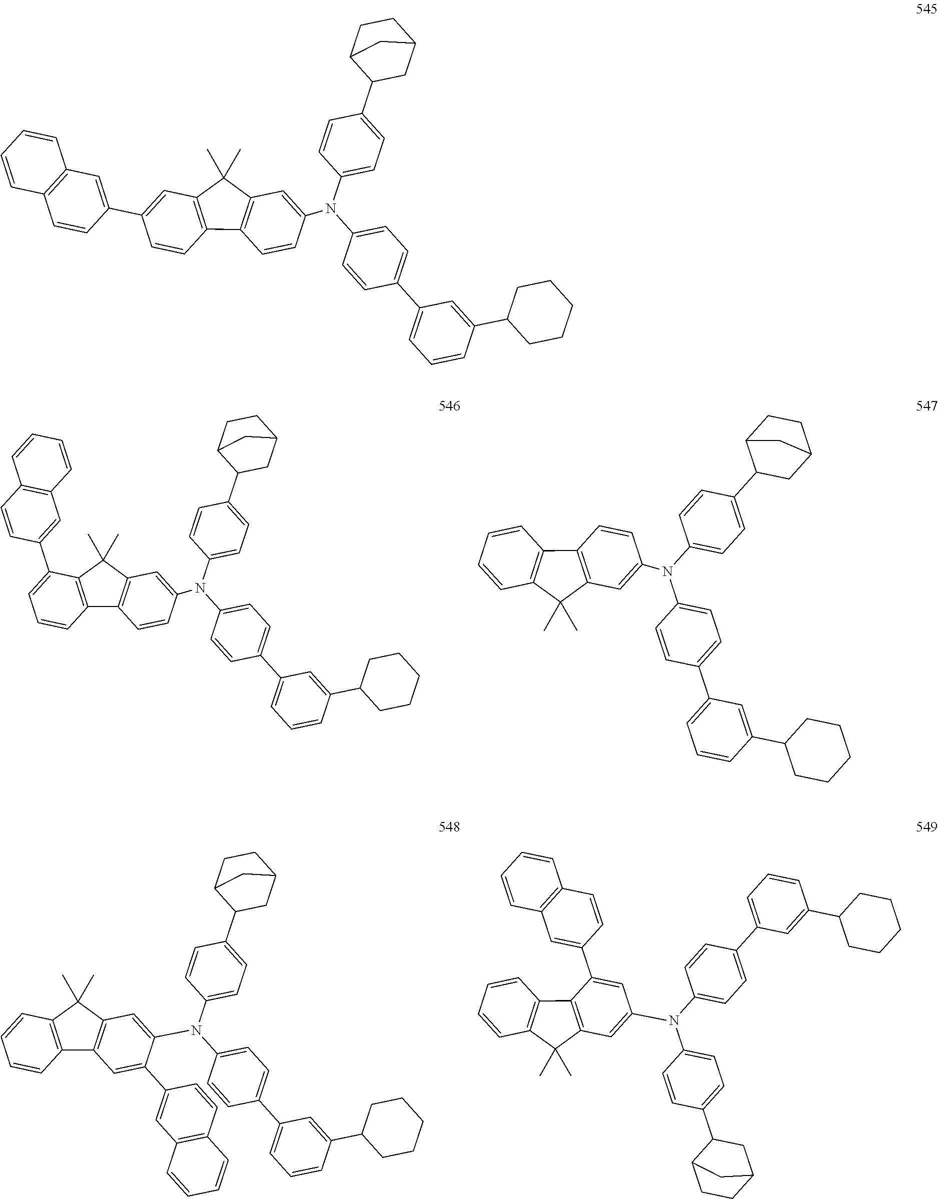 [SMILES: CC1(C)c2cc(-c3ccc4ccccc4c3)ccc2-c2ccc(N(c3ccc(-c4cccc(C5CCCCC5)c4)cc3)c3ccc(C4CC5CCC4C5)cc3)cc21 ; CC1(C)c2cc(N(c3ccc(-c4cccc(C5CCCCC5)c4)cc3)c3ccc(C4CC5CCC4C5)cc3)ccc2-c2cccc(-c3ccc4ccccc4c3)c21 ; CC1(C)c2ccccc2-c2ccc(N(c3ccc(-c4cccc(C5CCCCC5)c4)cc3)c3ccc(C4CC5CCC4C5)cc3)cc21 ; CC1(C)c2ccccc2-c2cc(-c3ccc4ccccc4c3)c(N(c3ccc(-c4cccc(C5CCCCC5)c4)cc3)c3ccc(C4CC5CCC4C5)cc3)cc21 ; CC1(C)c2ccccc2-c2c(-c3ccc4ccccc4c3)cc(N(c3ccc(-c4cccc(C5CCCCC5)c4)cc3)c3ccc(C4CC5CCC4C5)cc3)cc21] 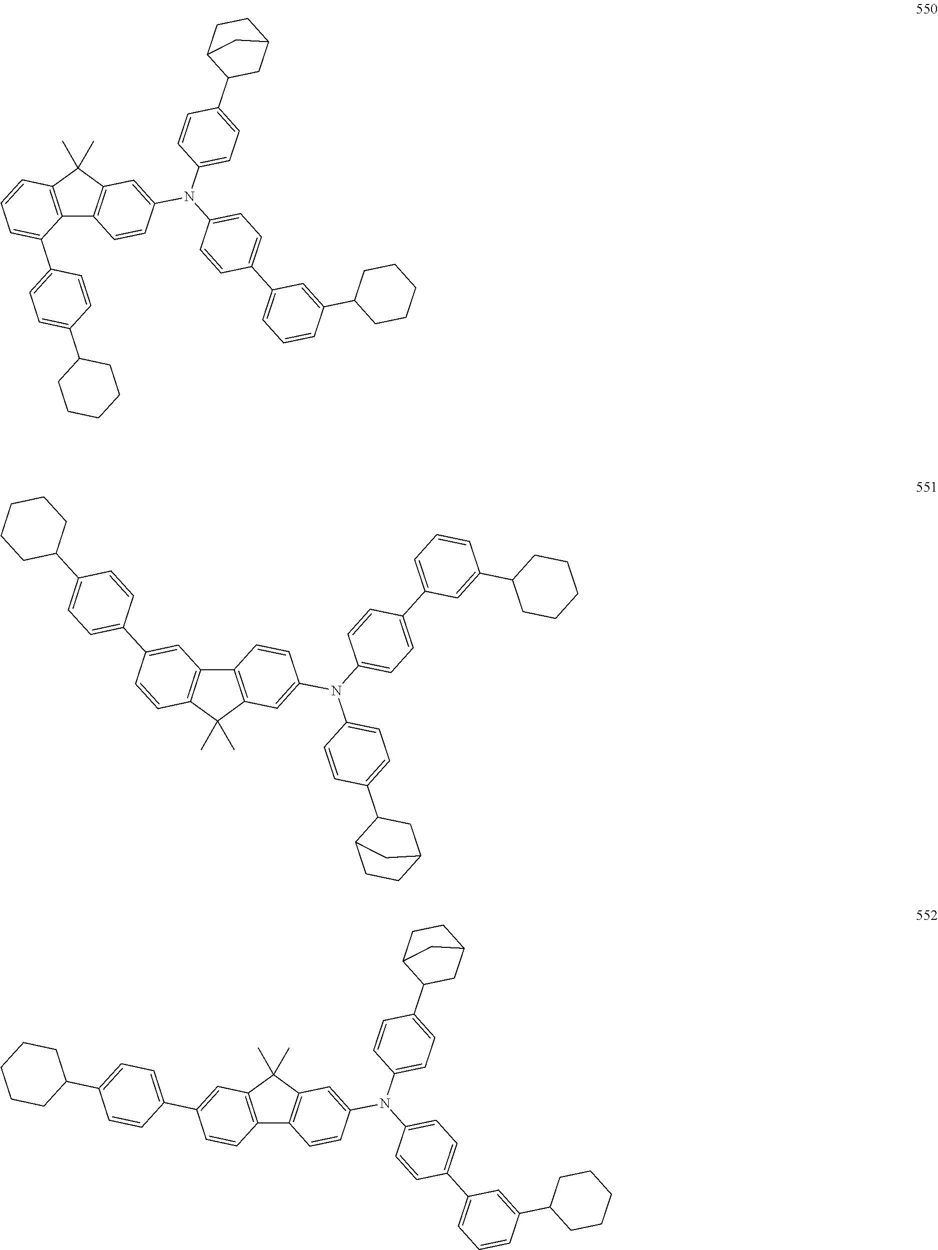 [SMILES: CC1(C)c2cc(N(c3ccc(-c4cccc(C5CCCCC5)c4)cc3)c3ccc(C4CC5CCC4C5)cc3)ccc2-c2c(-c3ccc(C4CCCCC4)cc3)cccc21 ; CC1(C)c2ccc(-c3ccc(C4CCCCC4)cc3)cc2-c2ccc(N(c3ccc(-c4cccc(C5CCCCC5)c4)cc3)c3ccc(C4CC5CCC4C5)cc3)cc21 ; CC1(C)c2cc(-c3ccc(C4CCCCC4)cc3)ccc2-c2ccc(N(c3ccc(-c4cccc(C5CCCCC5)c4)cc3)c3ccc(C4CC5CCC4C5)cc3)cc21] 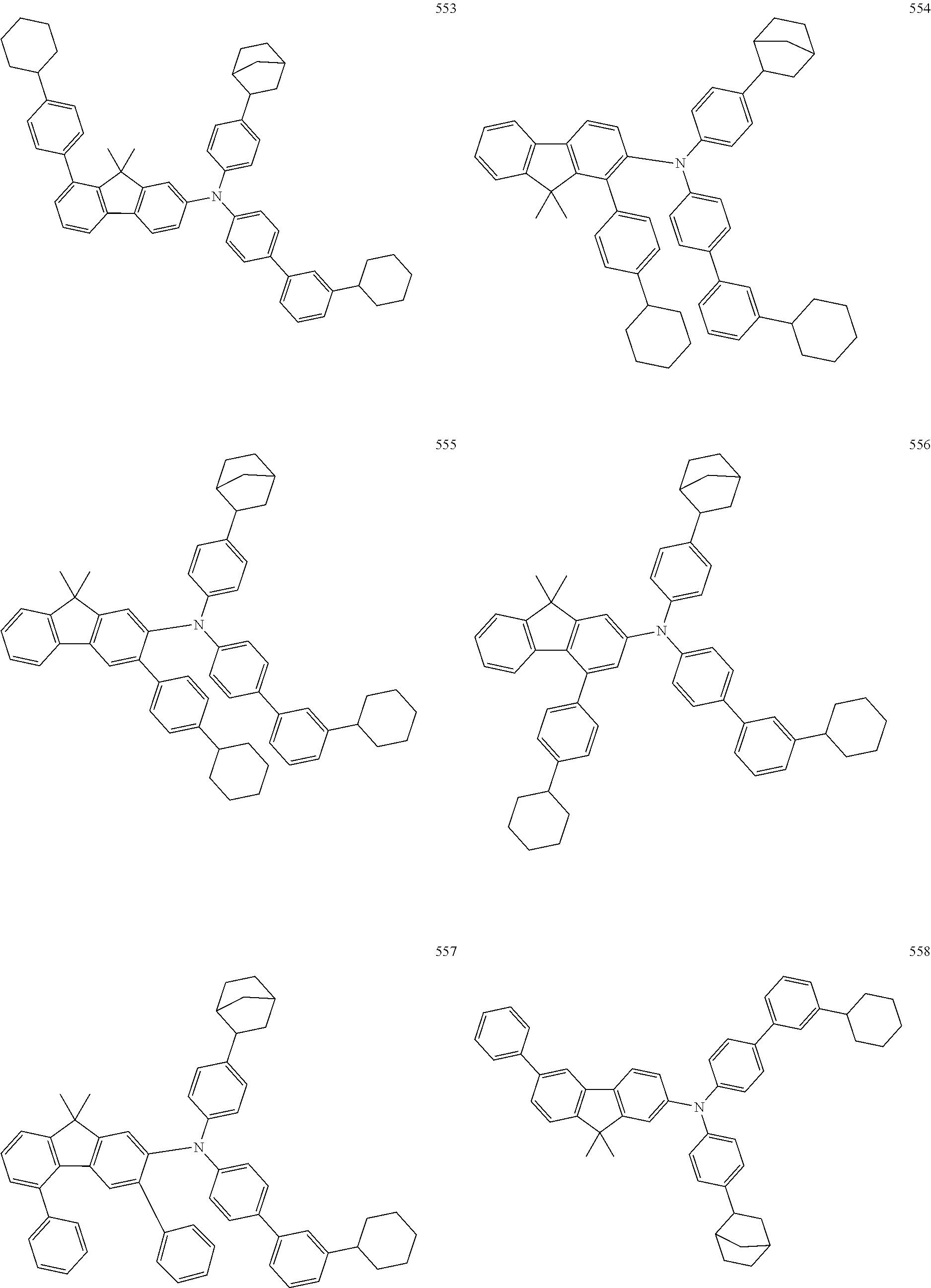 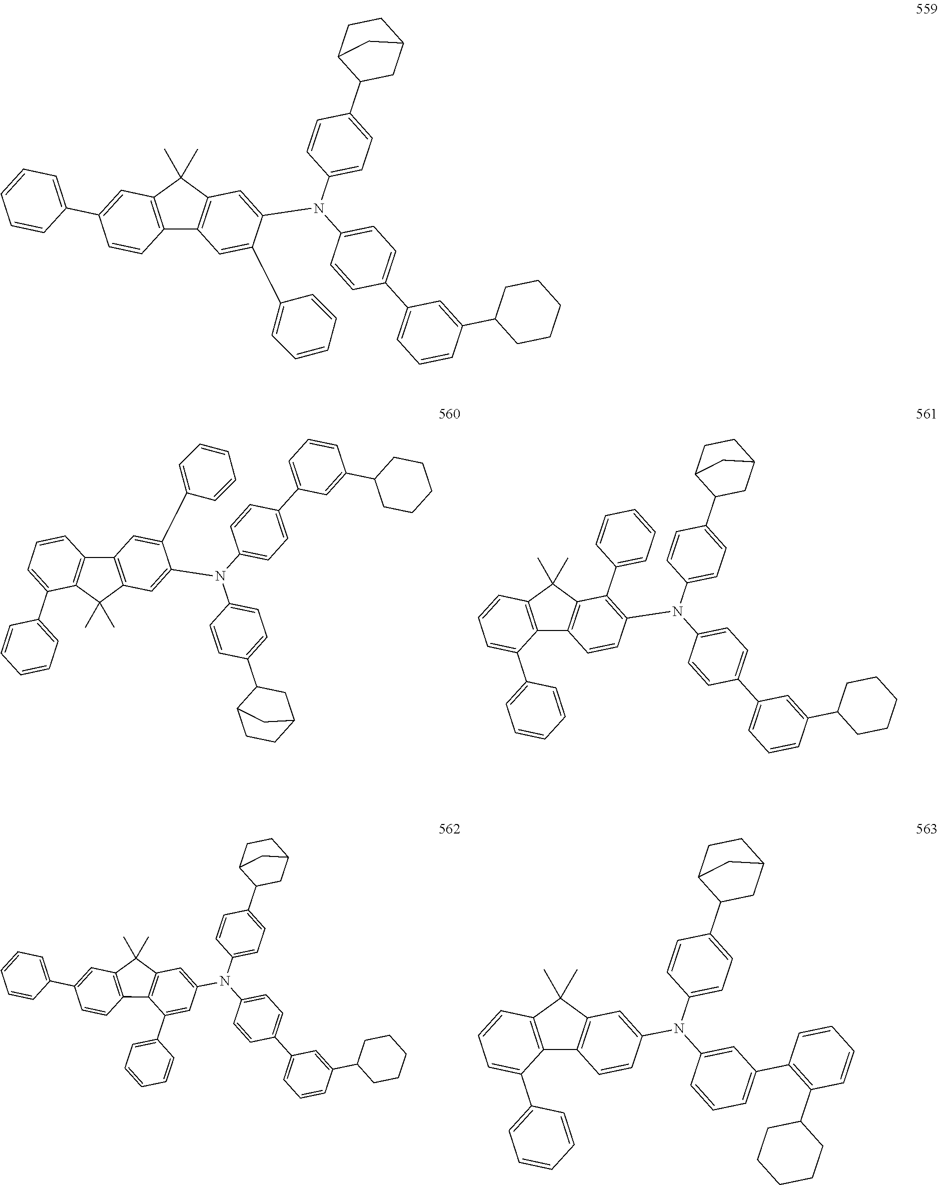 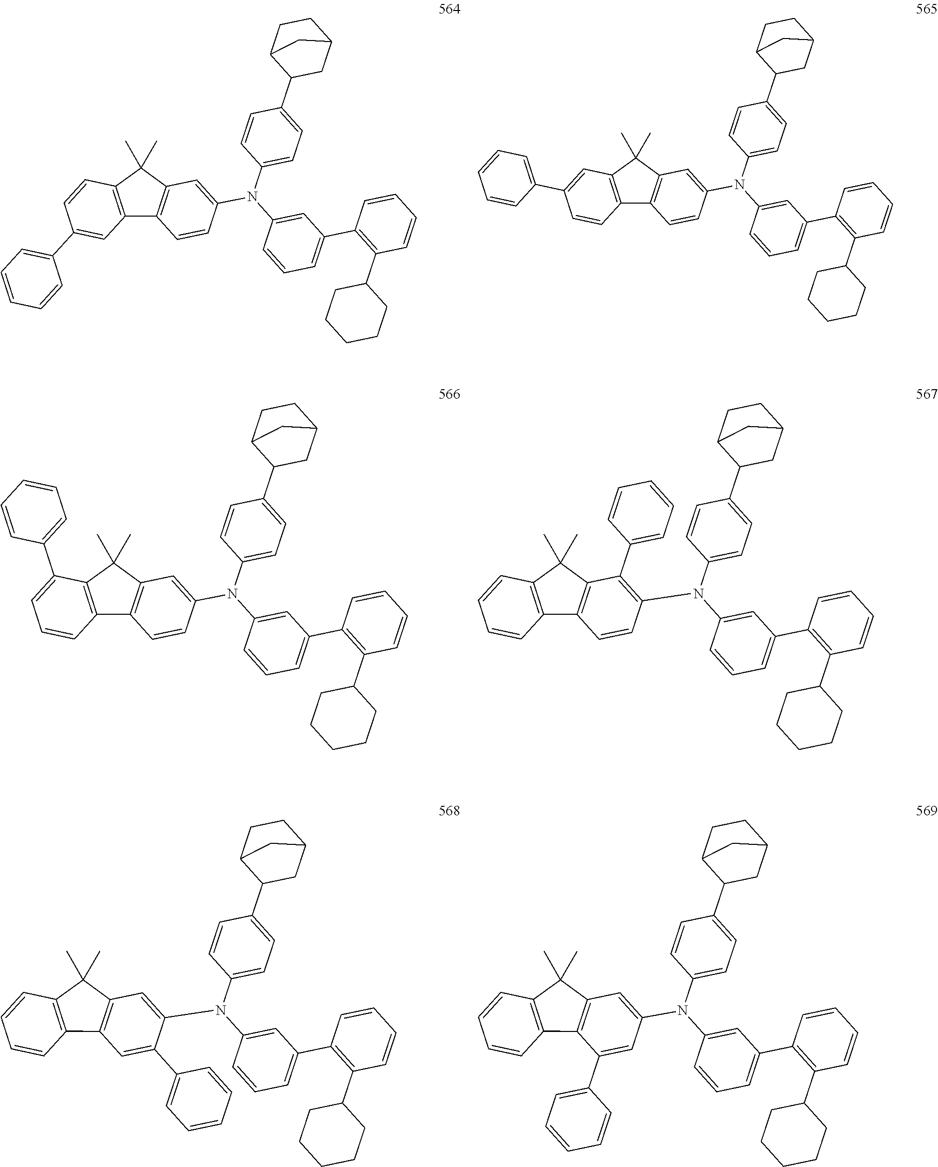 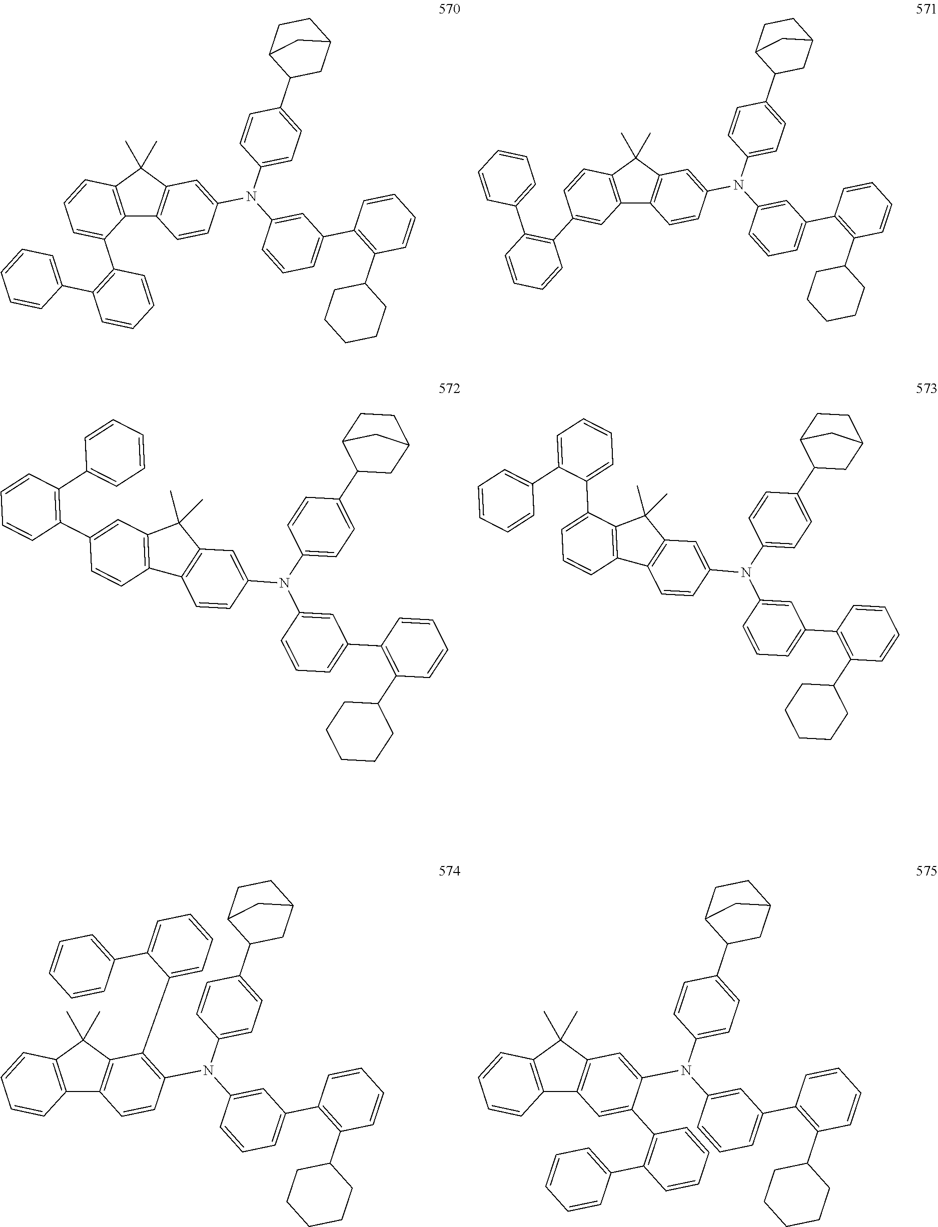 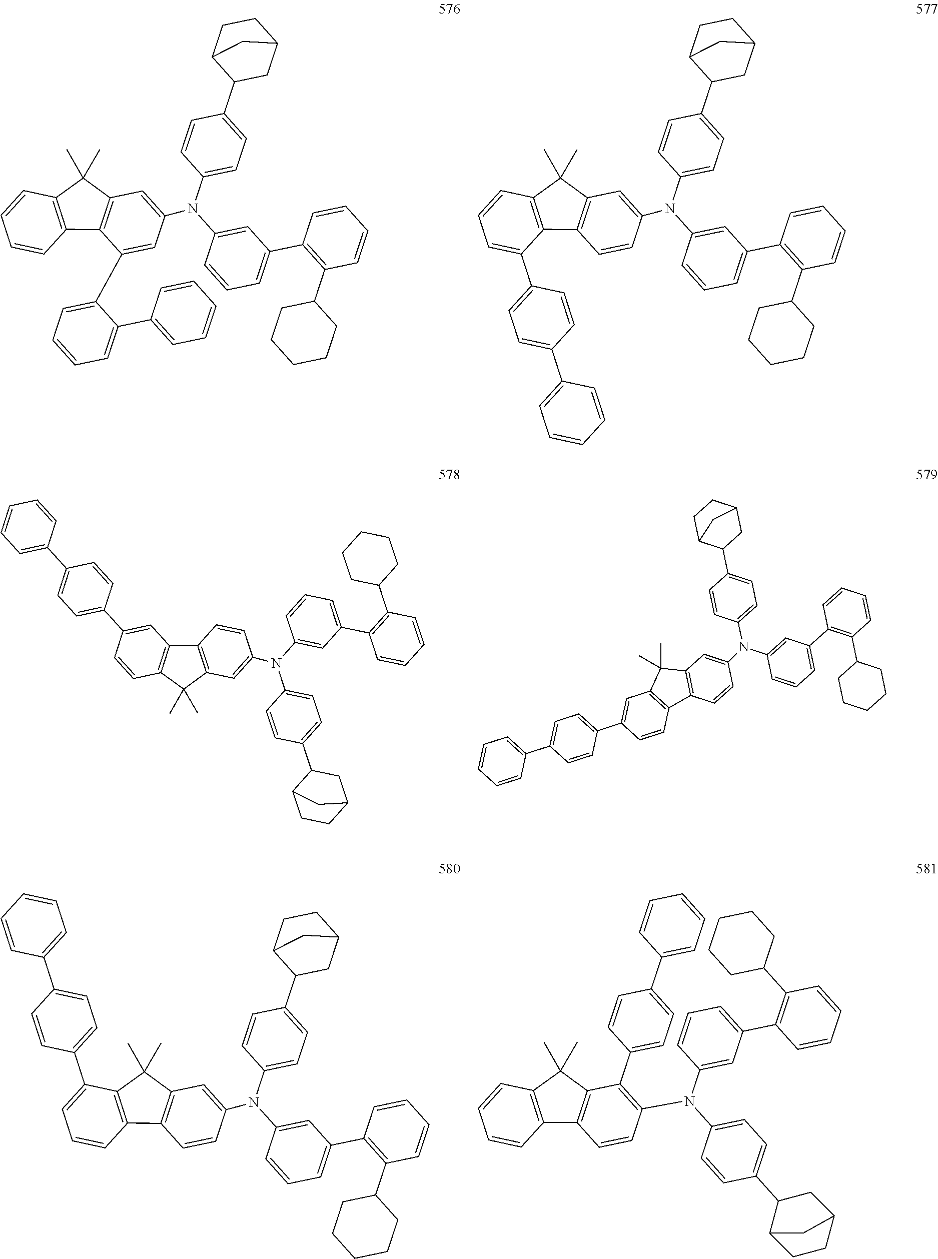 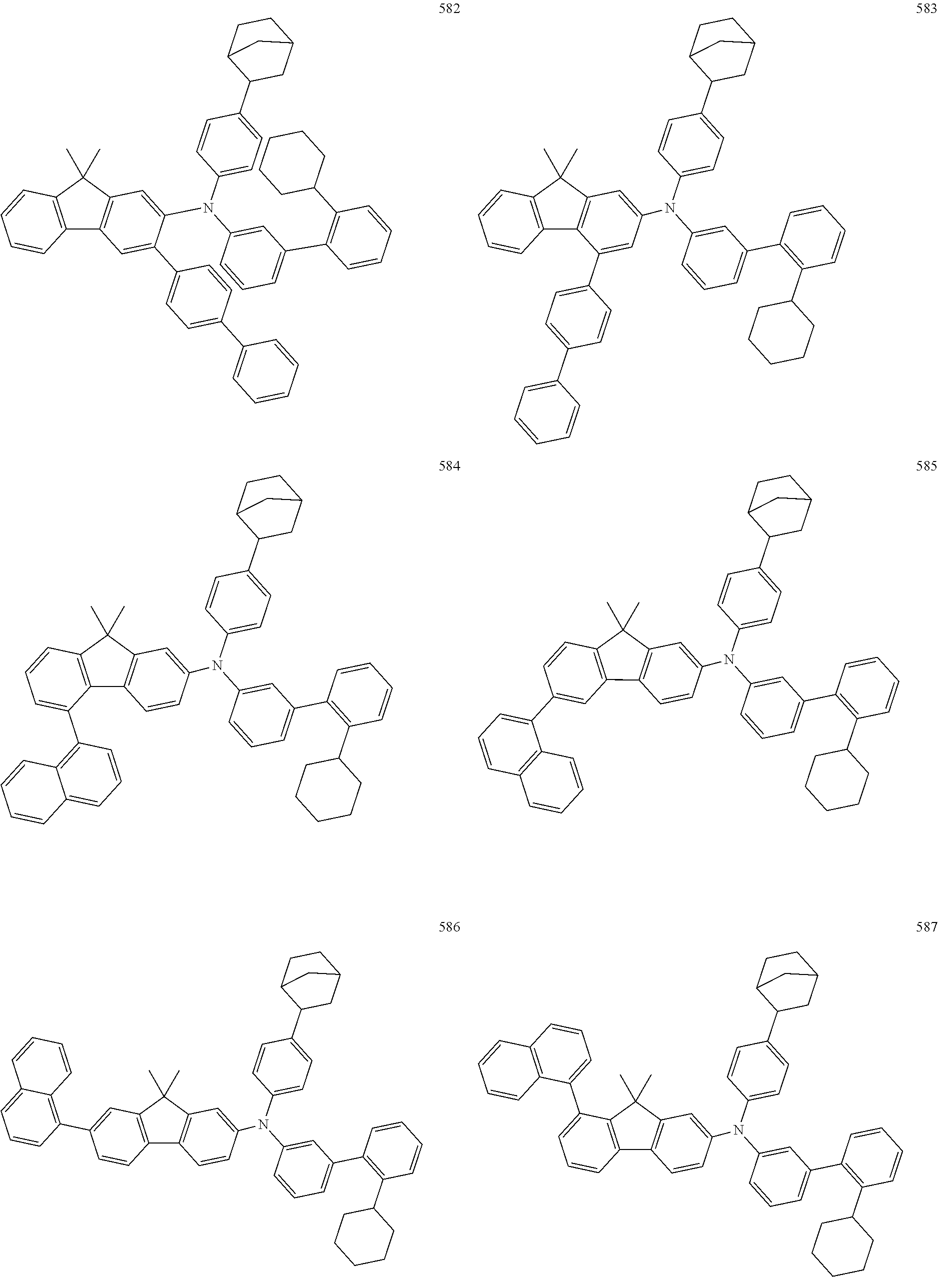 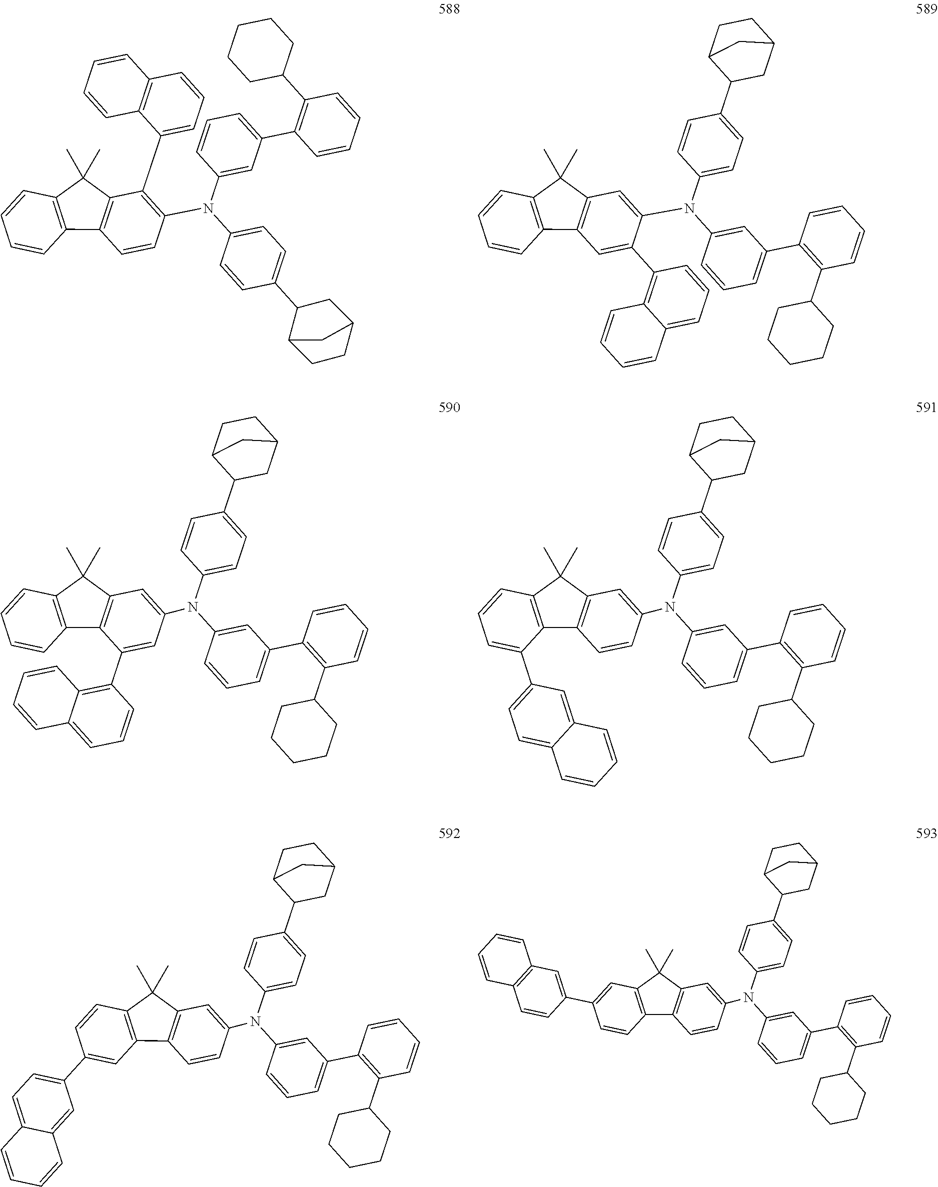 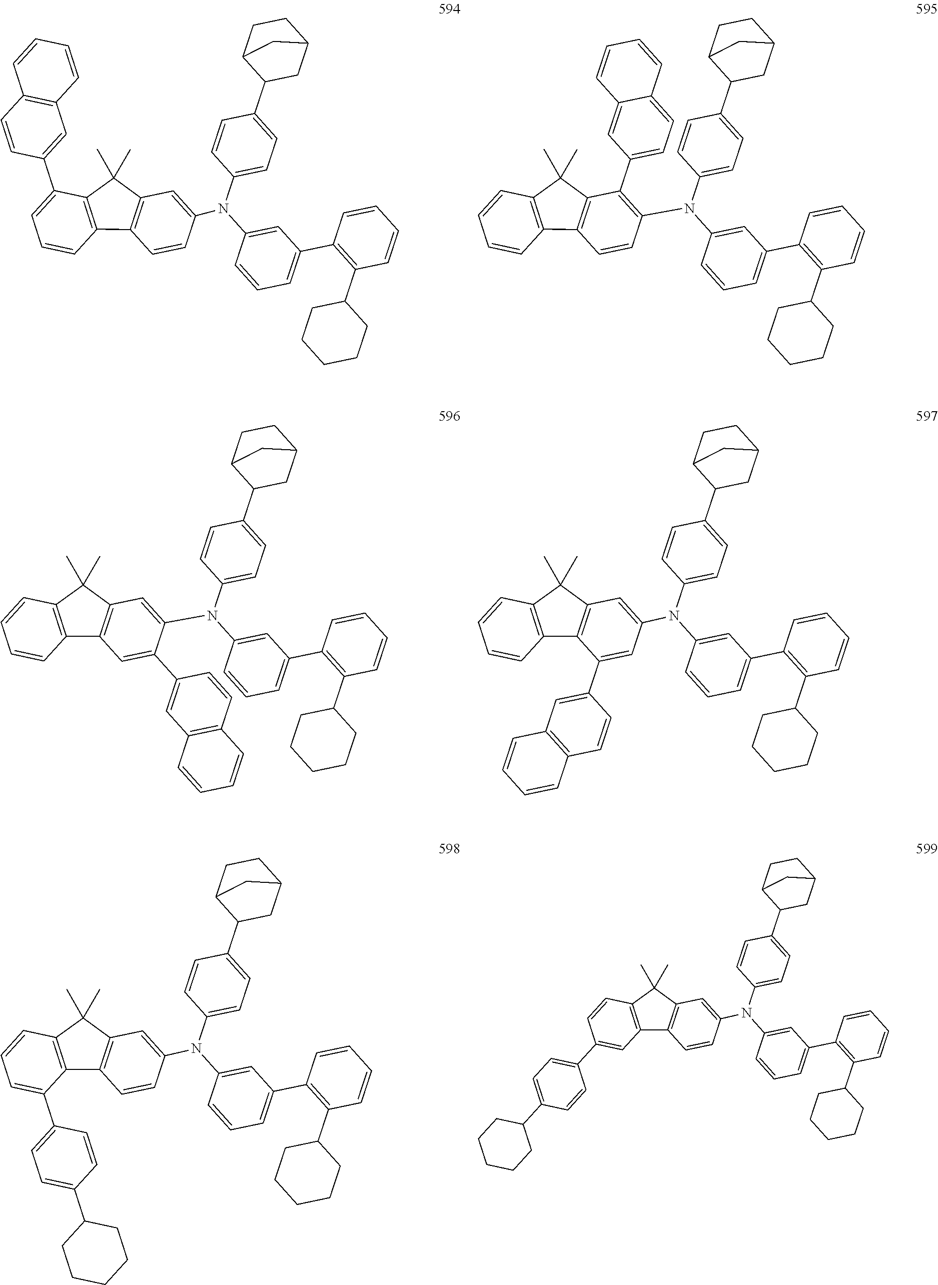 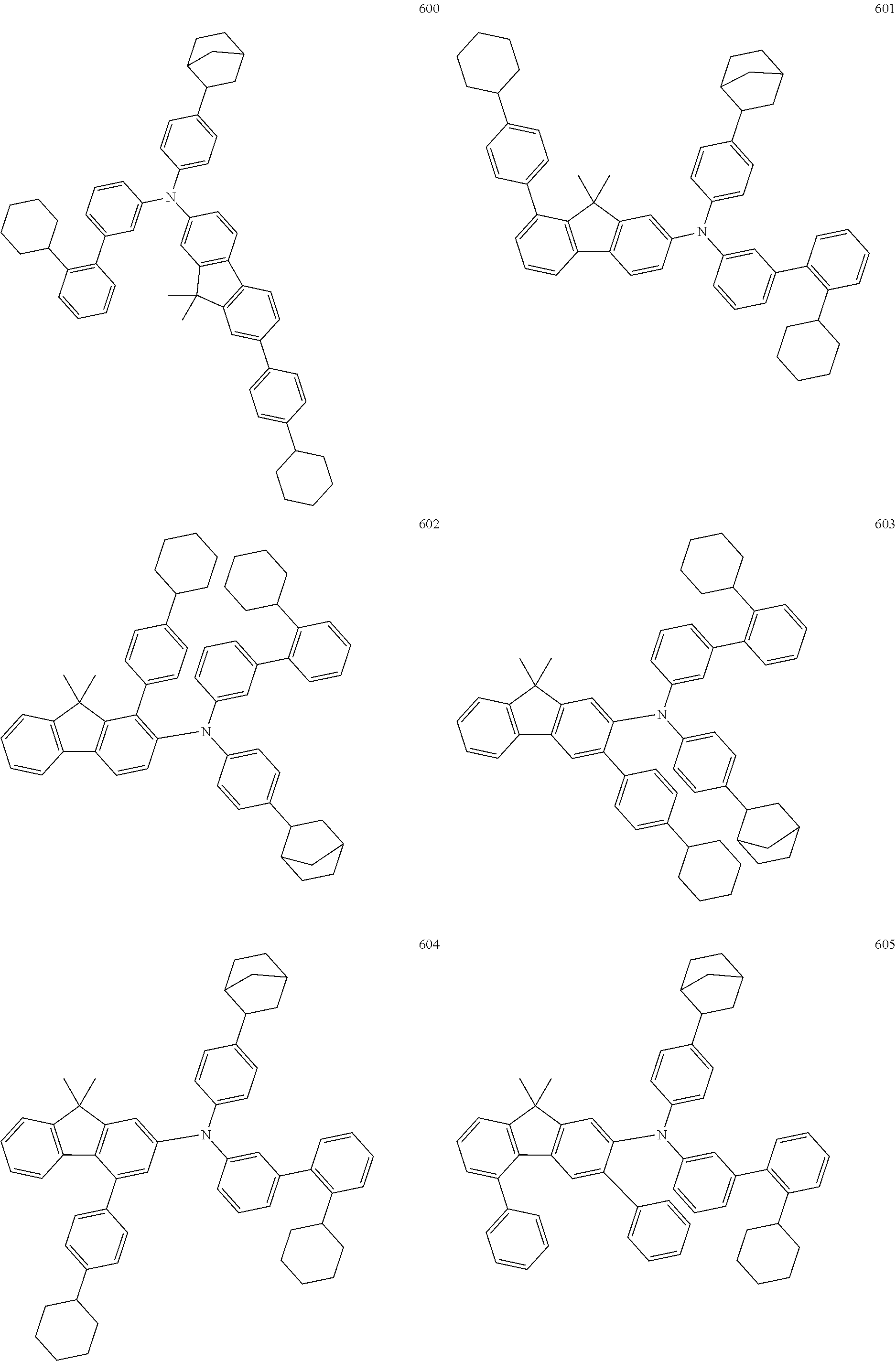 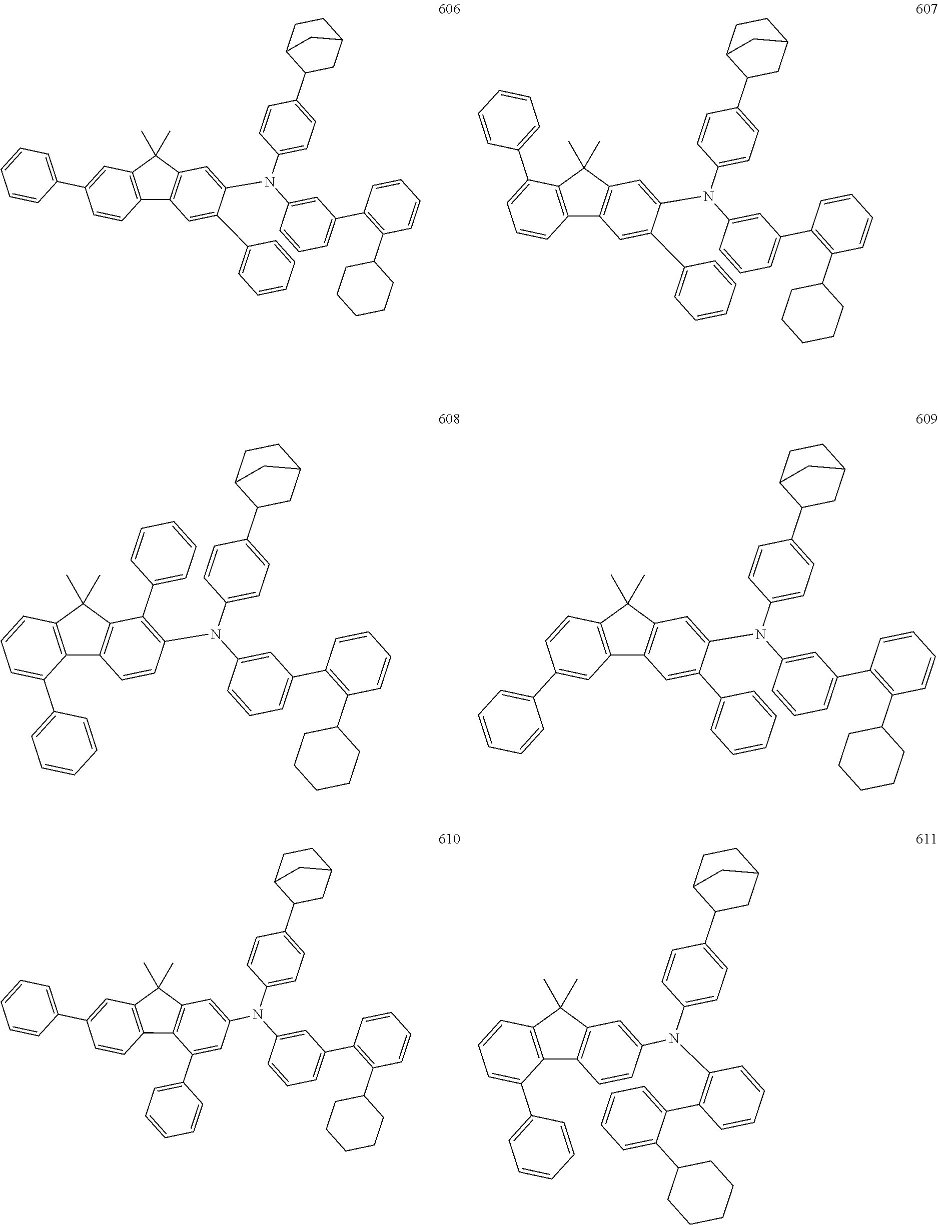 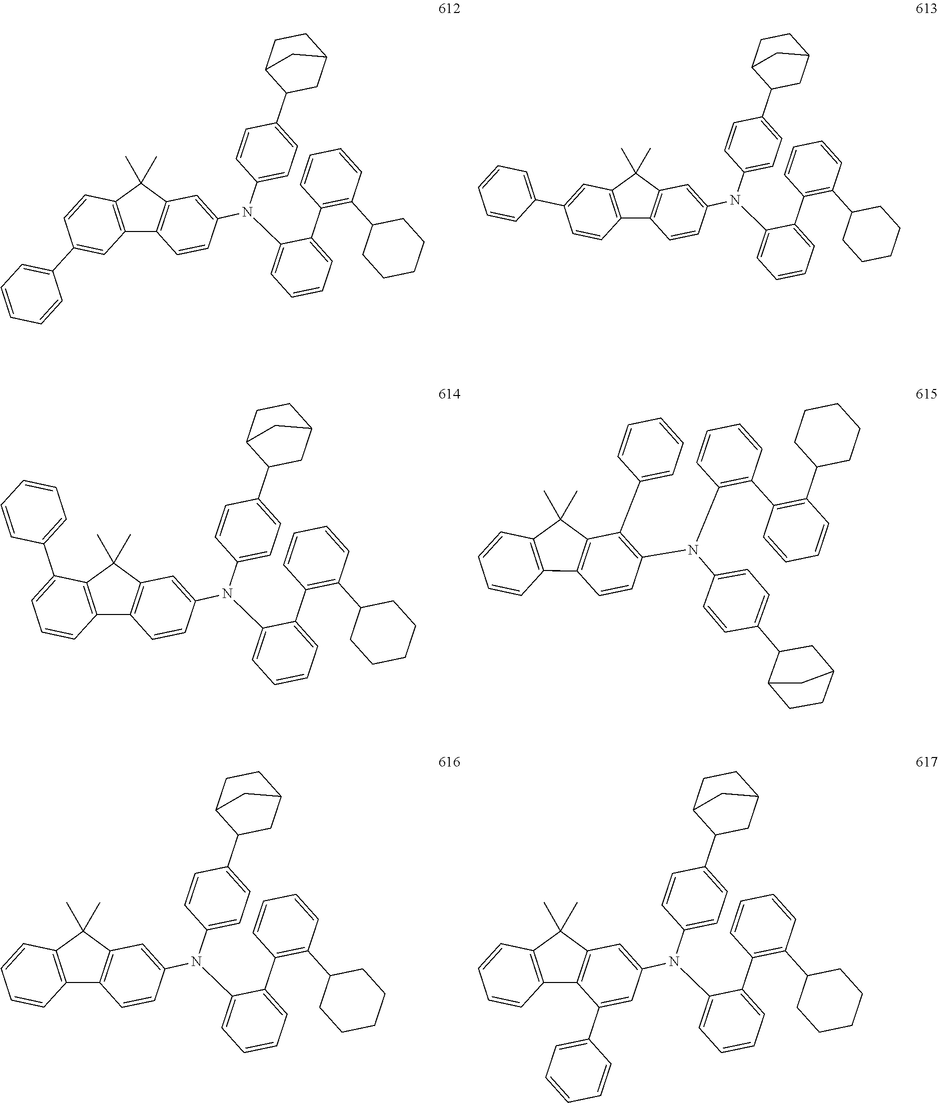 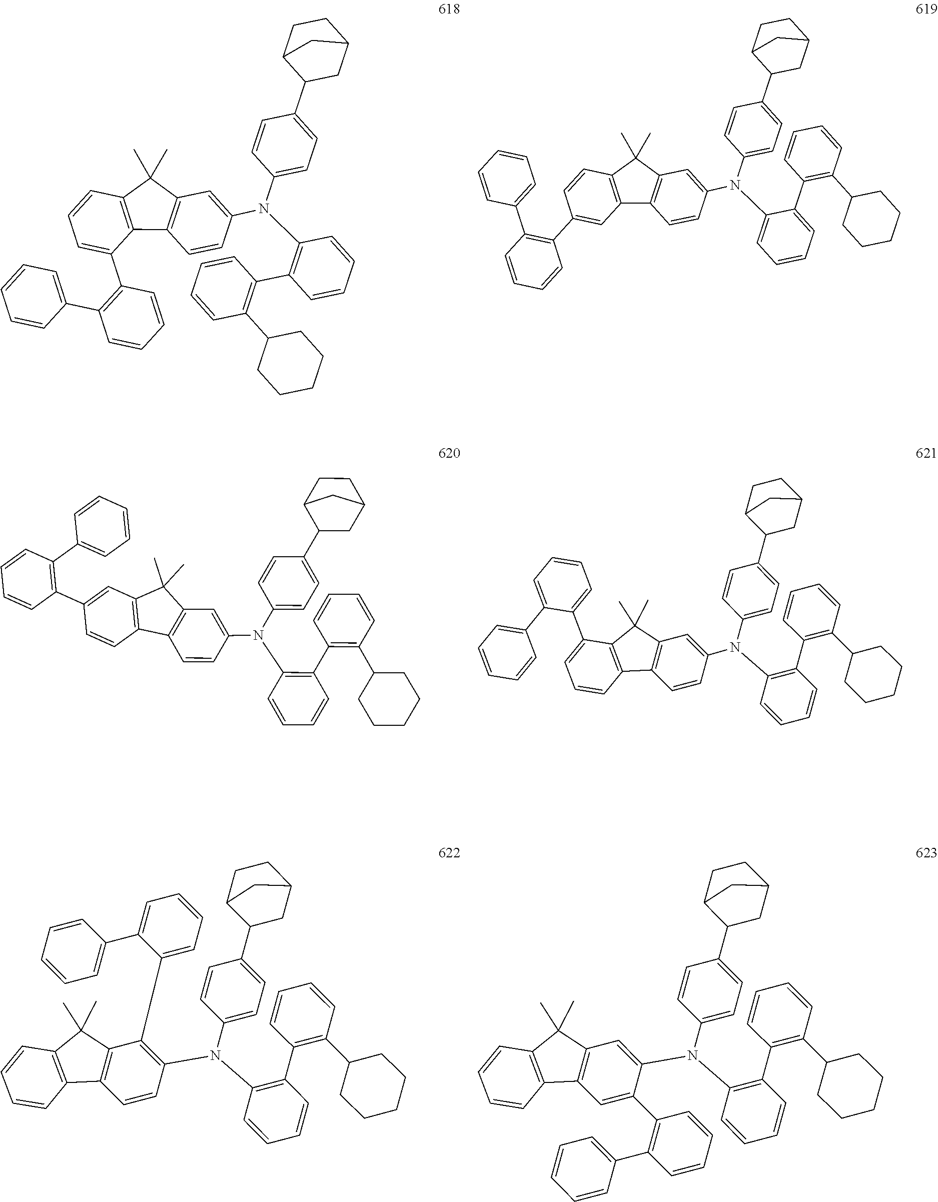 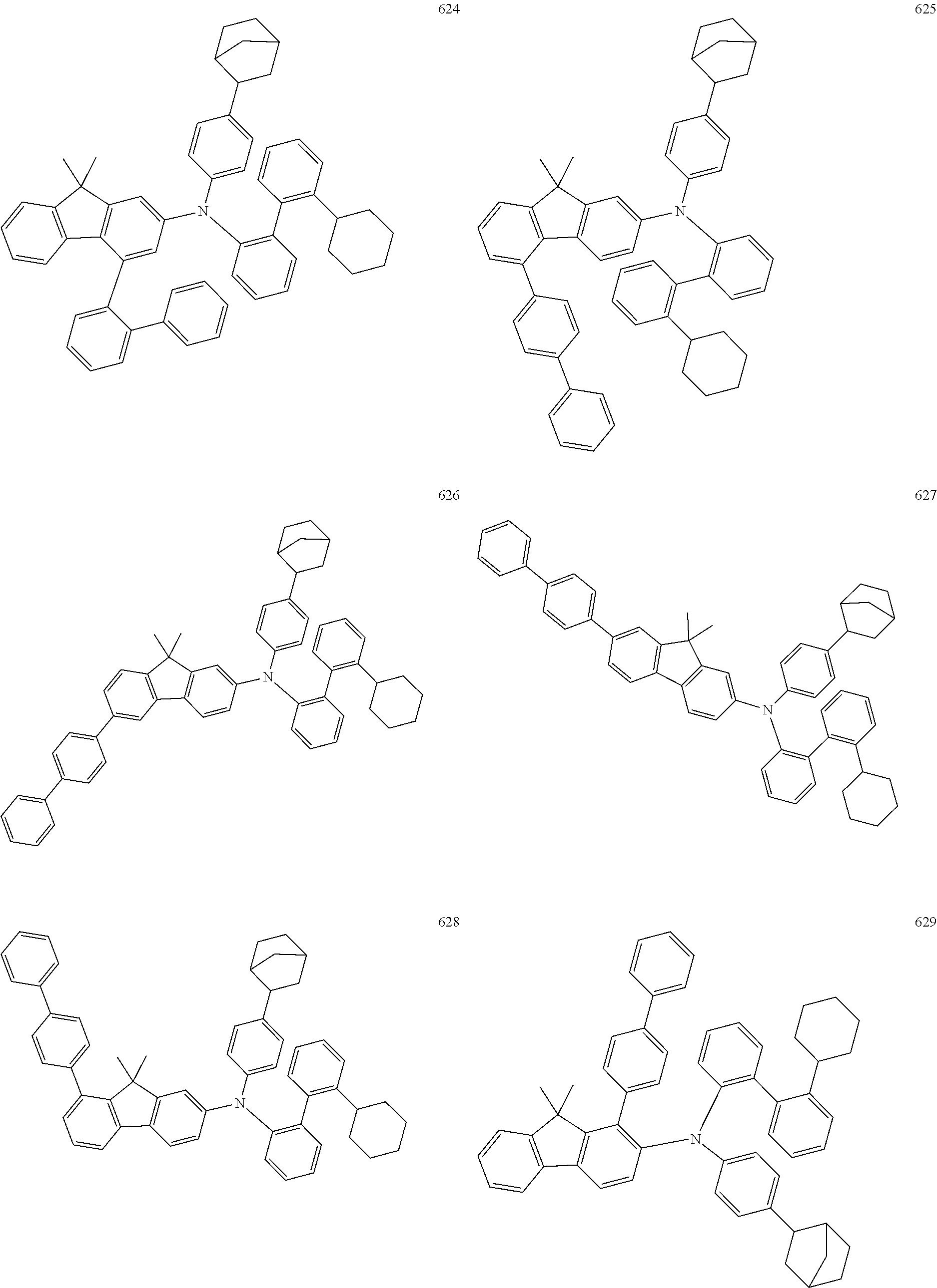 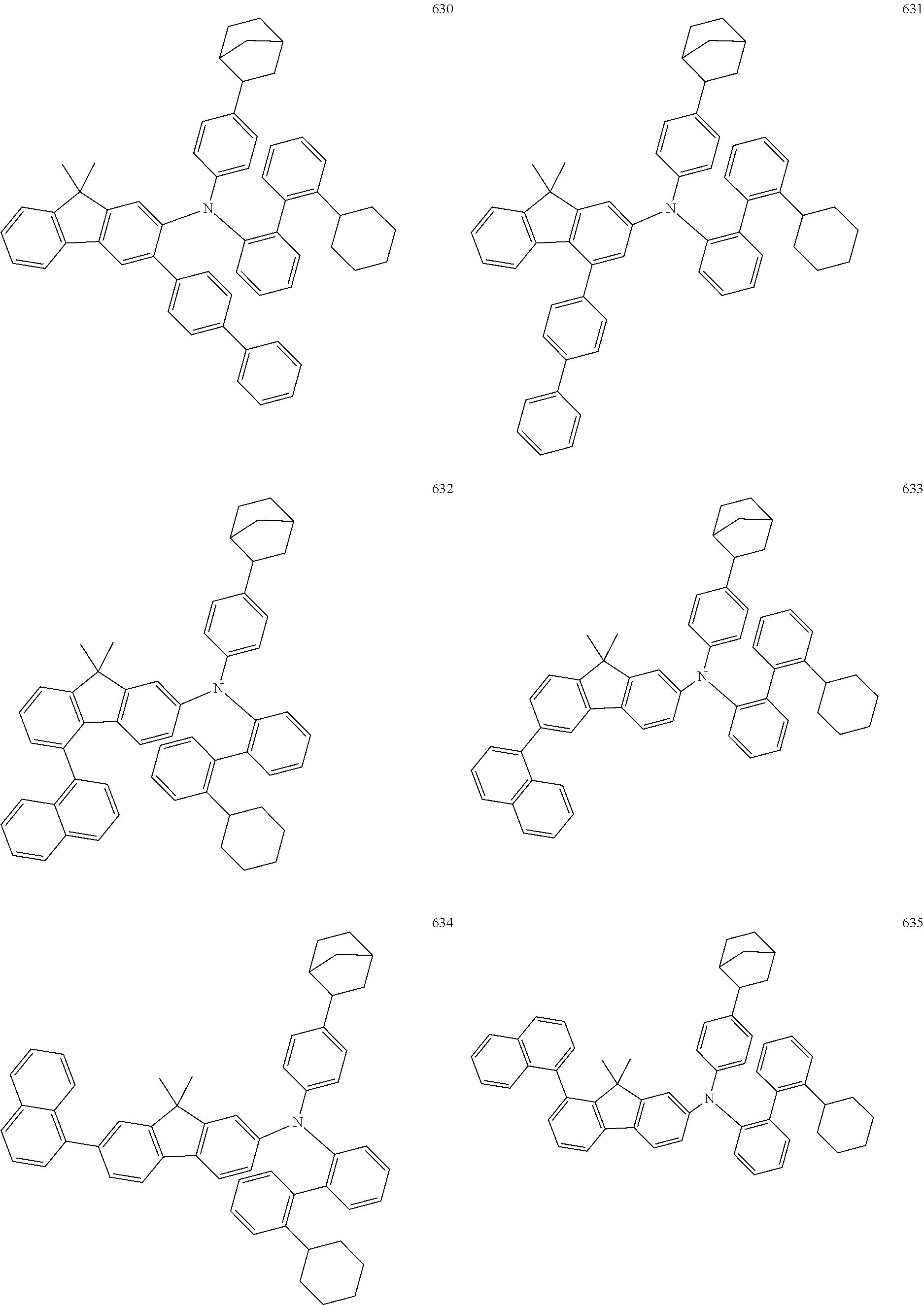 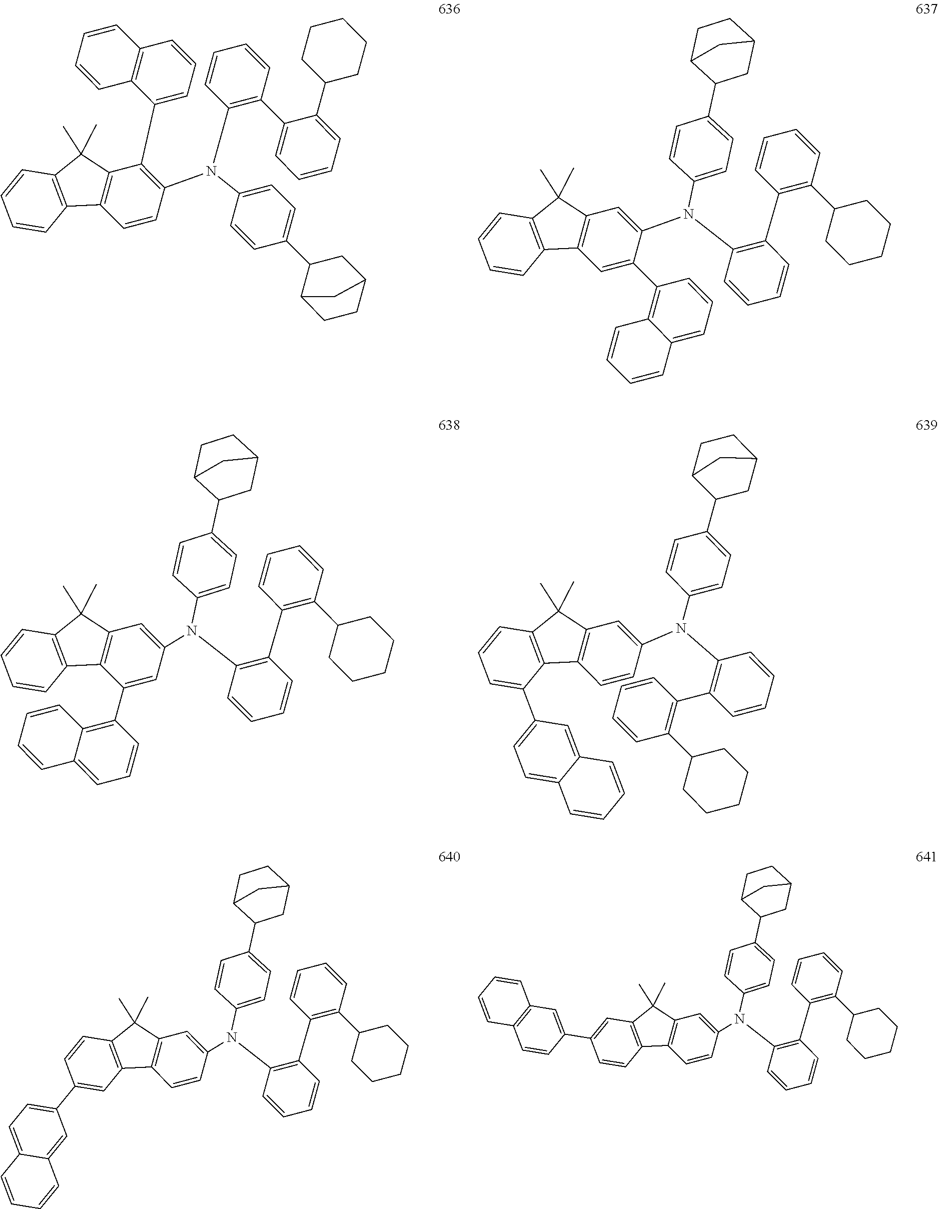 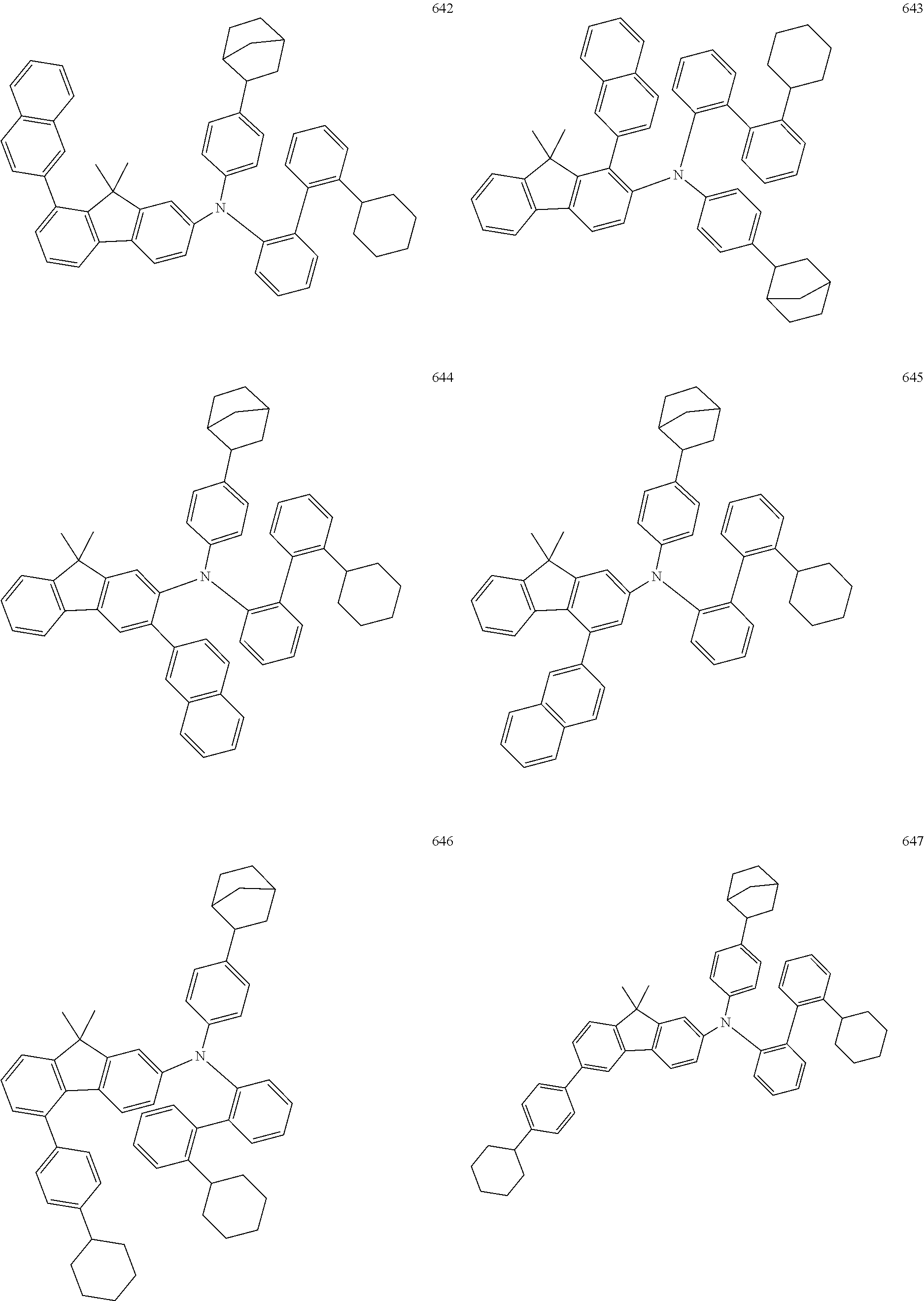 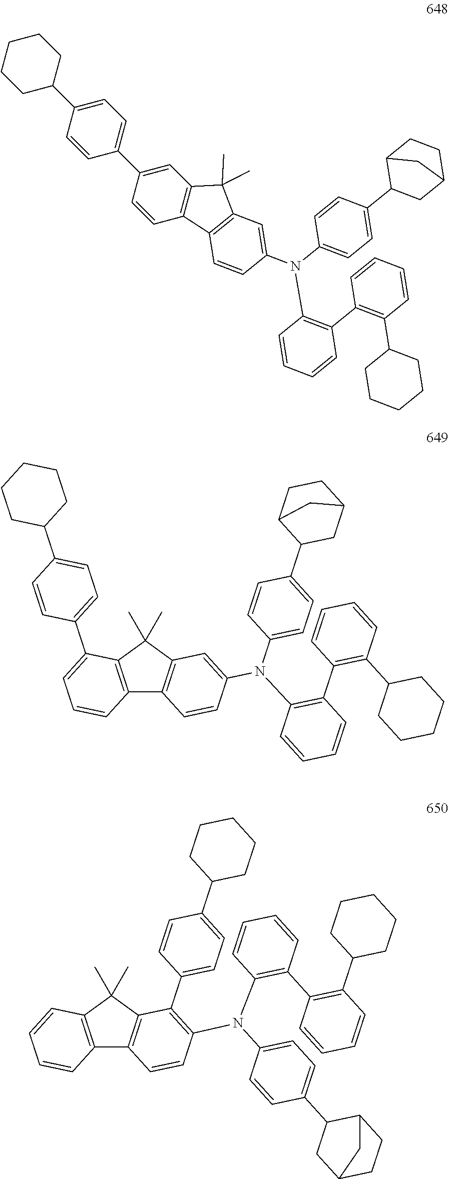 [SMILES: CC1(C)c2cc(-c3ccc(C4CCCCC4)cc3)ccc2-c2ccc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)ccc2-c2cccc(-c3ccc(C4CCCCC4)cc3)c21 ; CC1(C)c2ccccc2-c2ccc(N(c3ccc(C4CC5CCC4C5)cc3)c3ccccc3-c3ccccc3C3CCCCC3)c(-c3ccc(C4CCCCC4)cc3)c21] 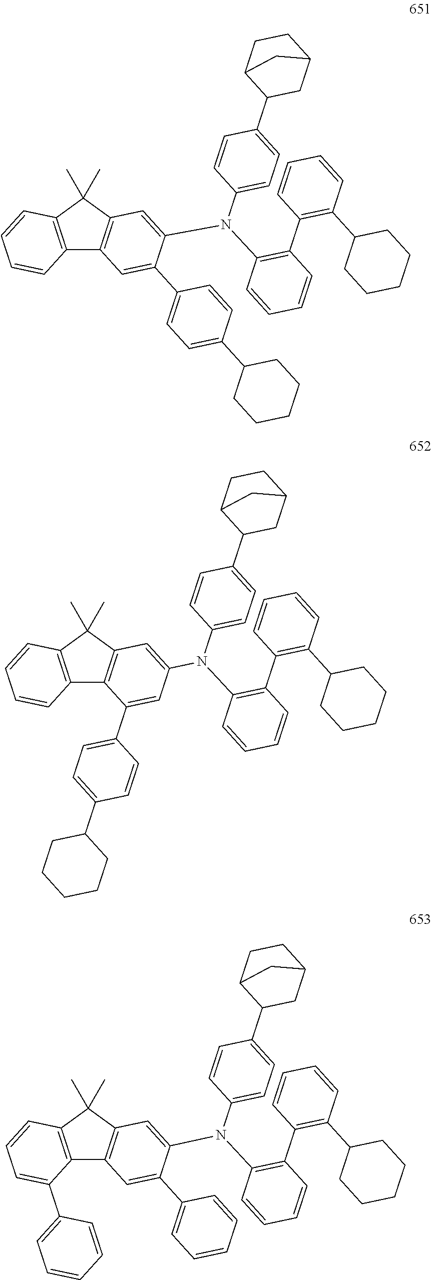 [SMILES: CC1(C)c2ccccc2-c2cc(-c3ccc(C4CCCCC4)cc3)c(N(c3ccc(C4CC5CCC4C5)cc3)c3ccccc3-c3ccccc3C3CCCCC3)cc21 ; CC1(C)c2ccccc2-c2c(-c3ccc(C4CCCCC4)cc3)cc(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)c(-c3ccccc3)cc2-c2c(-c3ccccc3)cccc21] 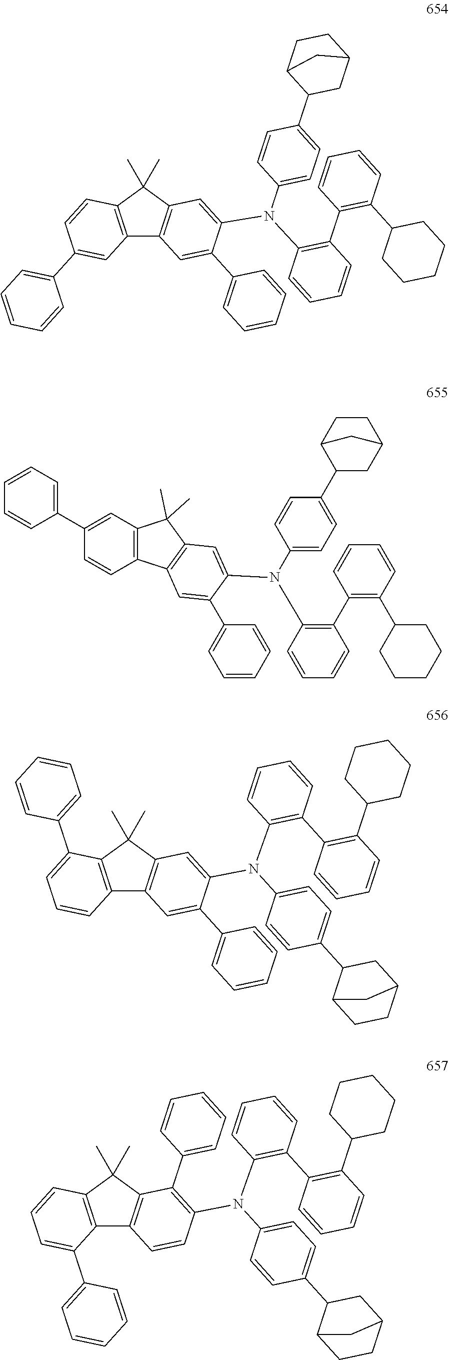 [SMILES: CC1(C)c2ccc(-c3ccccc3)cc2-c2cc(-c3ccccc3)c(N(c3ccc(C4CC5CCC4C5)cc3)c3ccccc3-c3ccccc3C3CCCCC3)cc21 ; CC1(C)c2cc(-c3ccccc3)ccc2-c2cc(-c3ccccc3)c(N(c3ccc(C4CC5CCC4C5)cc3)c3ccccc3-c3ccccc3C3CCCCC3)cc21 ; CC1(C)c2cc(N(c3ccc(C4CC5CCC4C5)cc3)c3ccccc3-c3ccccc3C3CCCCC3)c(-c3ccccc3)cc2-c2cccc(-c3ccccc3)c21 ; CC1(C)c2cccc(-c3ccccc3)c2-c2ccc(N(c3ccc(C4CC5CCC4C5)cc3)c3ccccc3-c3ccccc3C3CCCCC3)c(-c3ccccc3)c21] 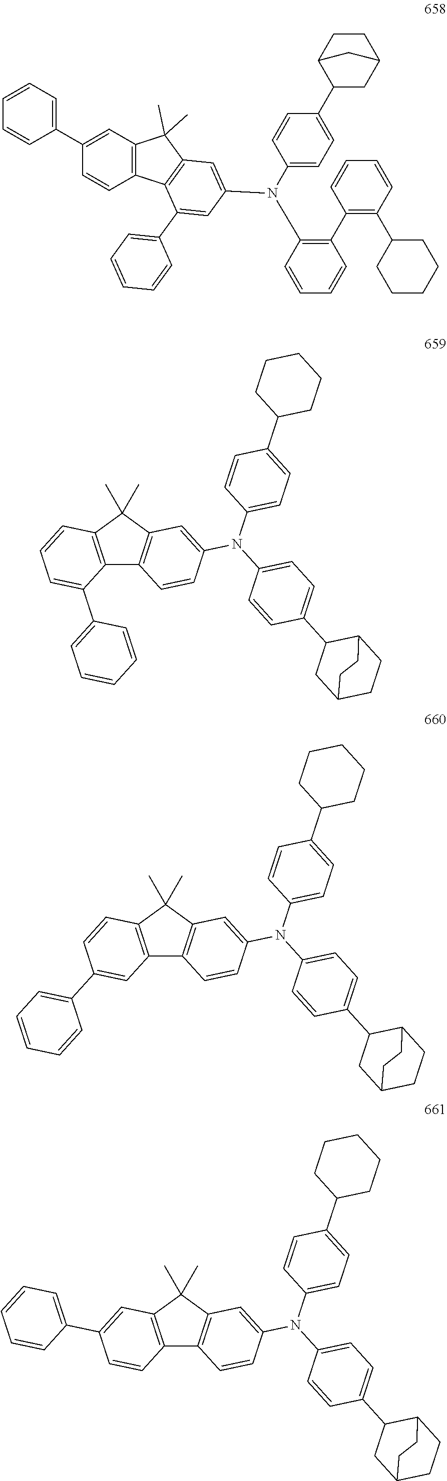 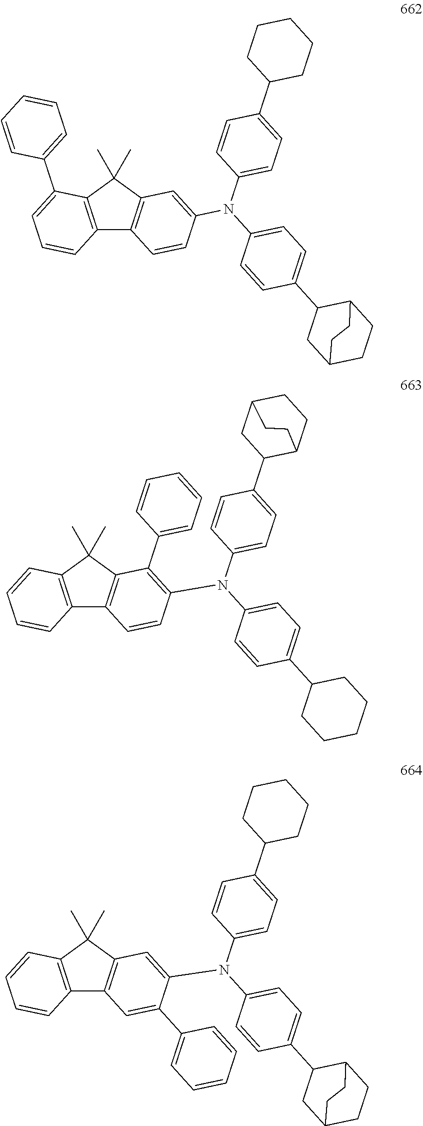 [SMILES: CC1(C)c2cc(N(c3ccc(C4CCCCC4)cc3)c3ccc(C4CC5CCC4CC5)cc3)ccc2-c2cccc(-c3ccccc3)c21 ; CC1(C)c2ccccc2-c2ccc(N(c3ccc(C4CCCCC4)cc3)c3ccc(C4CC5CCC4CC5)cc3)c(-c3ccccc3)c21 ; CC1(C)c2ccccc2-c2cc(-c3ccccc3)c(N(c3ccc(C4CCCCC4)cc3)c3ccc(C4CC5CCC4CC5)cc3)cc21] 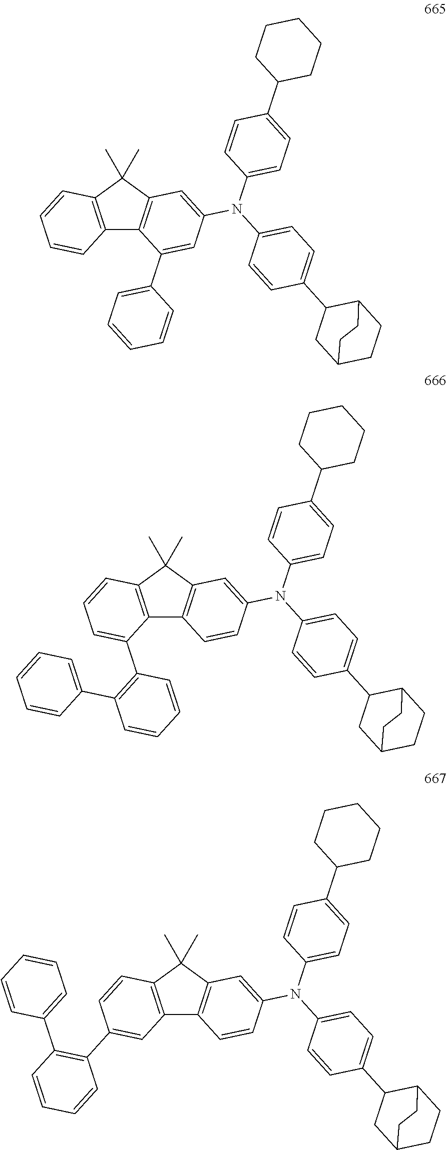 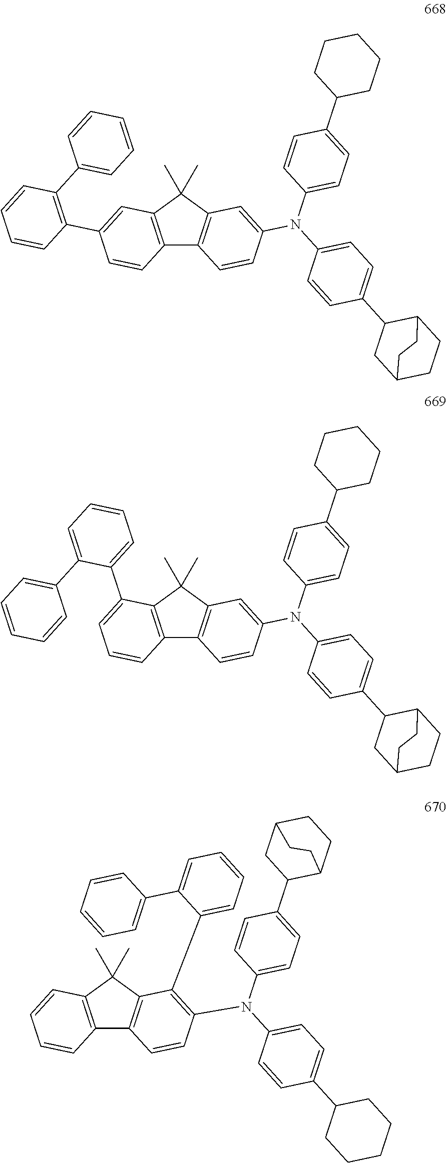 [SMILES: CC1(C)c2cc(-c3ccccc3-c3ccccc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccccc3-c3ccccc3)c21 ; CC1(C)c2ccccc2-c2ccc(N(c3ccc(C4CCCCC4)cc3)c3ccc(C4CC5CCC4CC5)cc3)c(-c3ccccc3-c3ccccc3)c21] 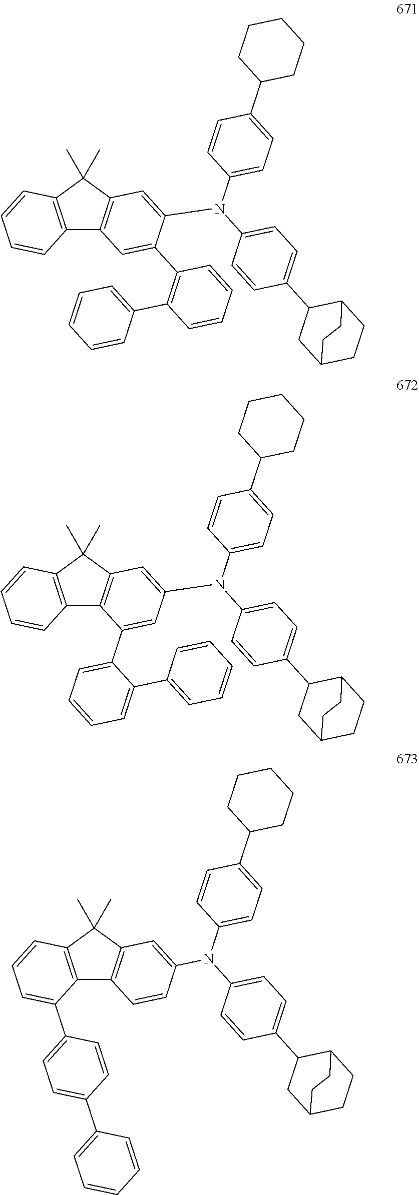 [SMILES: CC1(C)c2ccccc2-c2cc(-c3ccccc3-c3ccccc3)c(N(c3ccc(C4CCCCC4)cc3)c3ccc(C4CC5CCC4CC5)cc3)cc21 ; CC1(C)c2ccccc2-c2c(-c3ccccc3-c3ccccc3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc(-c4ccccc4)cc3)cccc21] 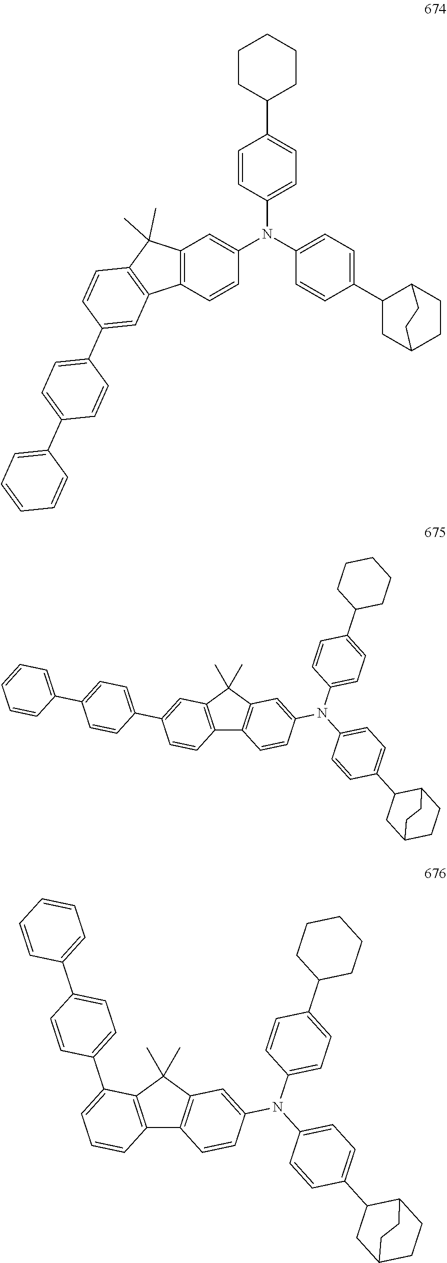 [SMILES: CC1(C)c2ccc(-c3ccc(-c4ccccc4)cc3)cc2-c2ccc(N(c3ccc(C4CCCCC4)cc3)c3ccc(C4CC5CCC4CC5)cc3)cc21 ; CC1(C)c2cc(-c3ccc(-c4ccccc4)cc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc(-c4ccccc4)cc3)c21] 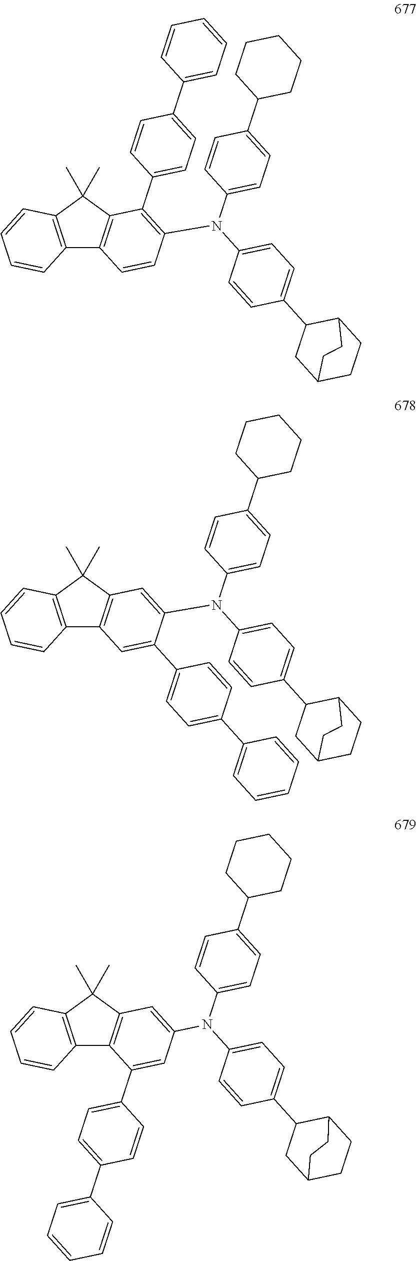 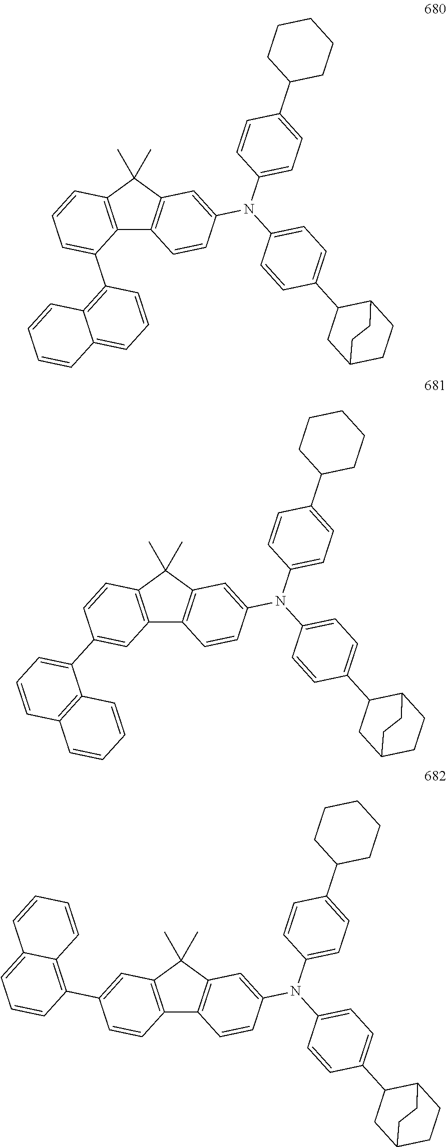 [SMILES: CC1(C)c2cc(N(c3ccc(C4CCCCC4)cc3)c3ccc(C4CC5CCC4CC5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CC5CCC4CC5)cc3)cc21 ; CC1(C)c2cc(-c3cccc4ccccc34)ccc2-c2ccc(N(c3ccc(C4CCCCC4)cc3)c3ccc(C4CC5CCC4CC5)cc3)cc21] 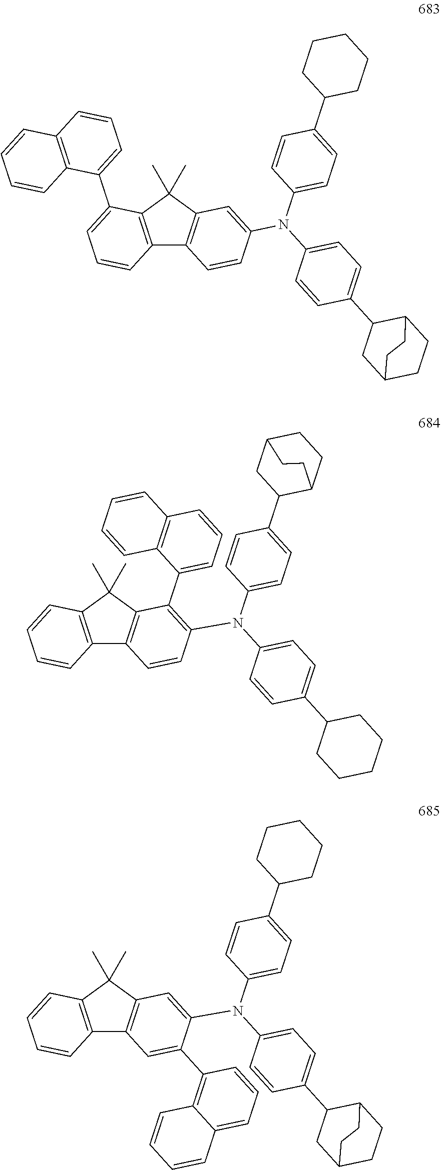 [SMILES: CC1(C)c2cc(N(c3ccc(C4CCCCC4)cc3)c3ccc(C4CC5CCC4CC5)cc3)ccc2-c2cccc(-c3cccc4ccccc34)c21 ; CC1(C)c2ccccc2-c2ccc(N(c3ccc(C4CCCCC4)cc3)c3ccc(C4CC5CCC4CC5)cc3)c(-c3cccc4ccccc34)c21 ; CC1(C)c2ccccc2-c2cc(-c3cccc4ccccc34)c(N(c3ccc(C4CCCCC4)cc3)c3ccc(C4CC5CCC4CC5)cc3)cc21] 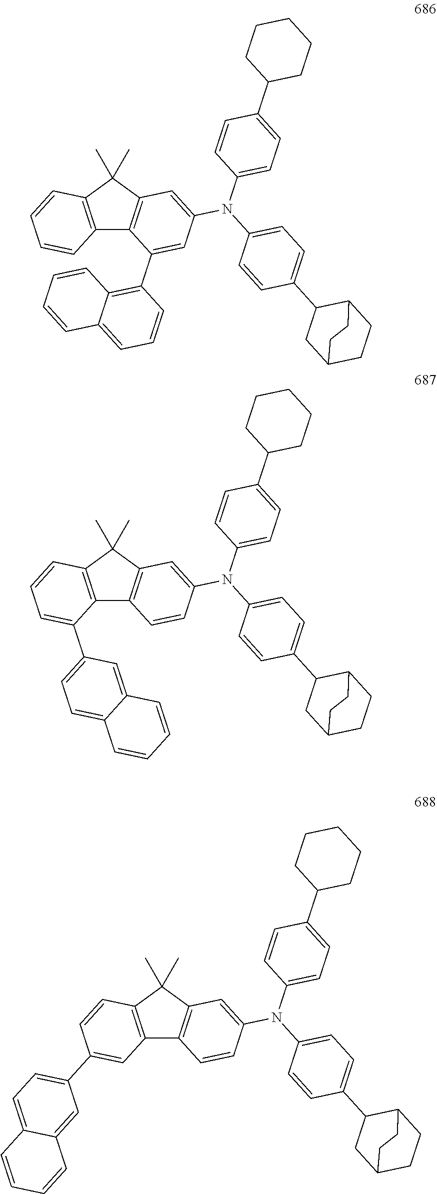 [SMILES: CC1(C)c2ccccc2-c2c(-c3cccc4ccccc34)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CC5CCC4CC5)cc3)cc21] 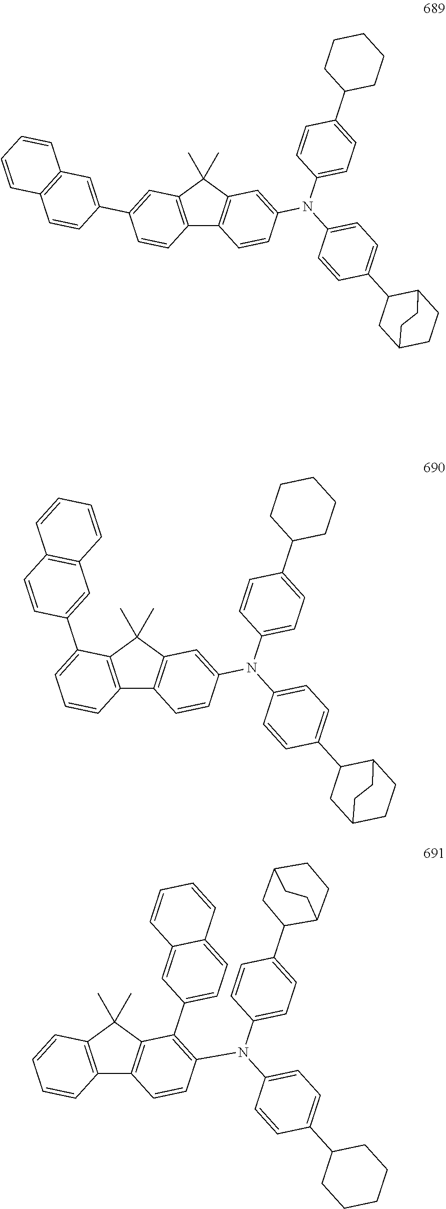 [SMILES: CC1(C)c2cc(-c3ccc4ccccc4c3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc4ccccc4c3)c21 ; CC1(C)c2ccccc2-c2ccc(N(c3ccc(C4CCCCC4)cc3)c3ccc(C4CC5CCC4CC5)cc3)c(-c3ccc4ccccc4c3)c21] 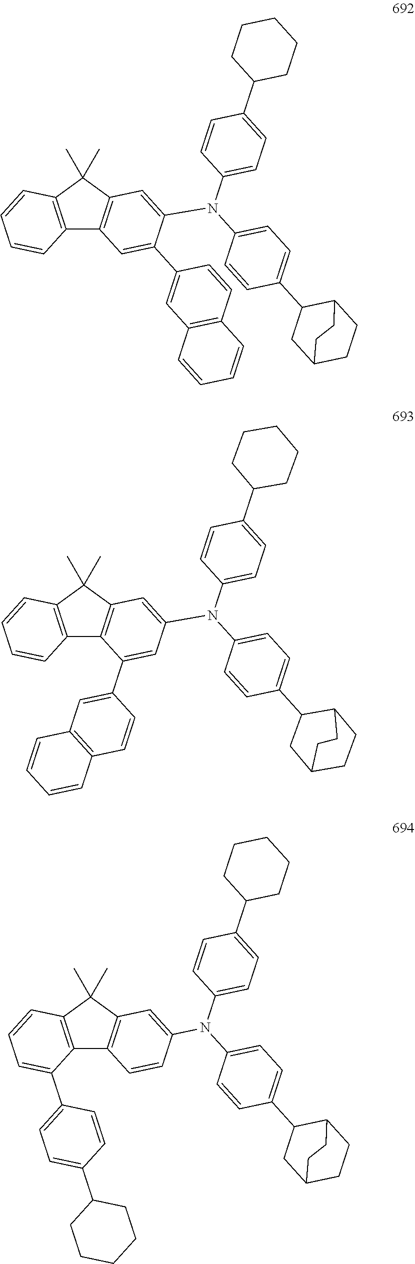 [SMILES: CC1(C)c2ccccc2-c2cc(-c3ccc4ccccc4c3)c(N(c3ccc(C4CCCCC4)cc3)c3ccc(C4CC5CCC4CC5)cc3)cc21 ; CC1(C)c2ccccc2-c2c(-c3ccc4ccccc4c3)cc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2c(-c3ccc(C4CCCCC4)cc3)cccc21] 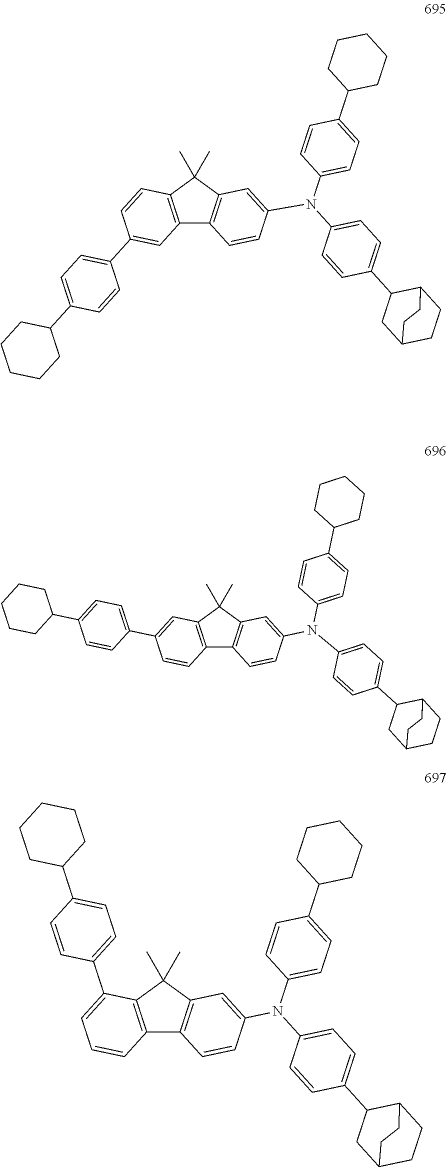 [SMILES: CC1(C)c2ccc(-c3ccc(C4CCCCC4)cc3)cc2-c2ccc(N(c3ccc(C4CCCCC4)cc3)c3ccc(C4CC5CCC4CC5)cc3)cc21 ; CC1(C)c2cc(-c3ccc(C4CCCCC4)cc3)ccc2-c2ccc(N(c3ccc(C4CCCCC4)cc3)c3ccc(C4CC5CCC4CC5)cc3)cc21 ; CC1(C)c2cc(N(c3ccc(C4CCCCC4)cc3)c3ccc(C4CC5CCC4CC5)cc3)ccc2-c2cccc(-c3ccc(C4CCCCC4)cc3)c21] 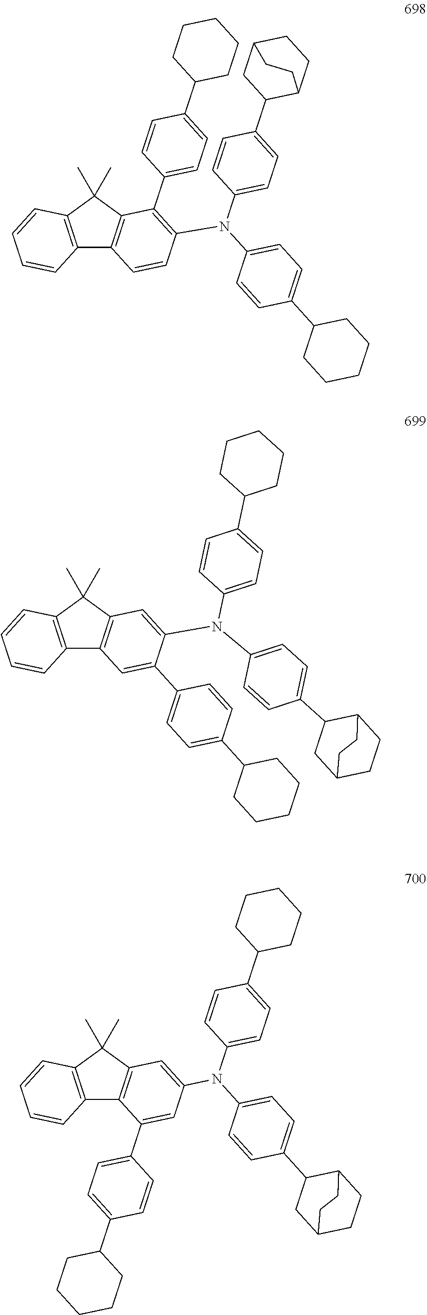 [SMILES: CC1(C)c2ccccc2-c2ccc(N(c3ccc(C4CCCCC4)cc3)c3ccc(C4CC5CCC4CC5)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C4CC5CCC4CC5)cc3)cc21 ; CC1(C)c2ccccc2-c2c(-c3ccc(C4CCCCC4)cc3)cc(N(c3ccc(C4CCCCC4)cc3)c3ccc(C4CC5CCC4CC5)cc3)cc21] 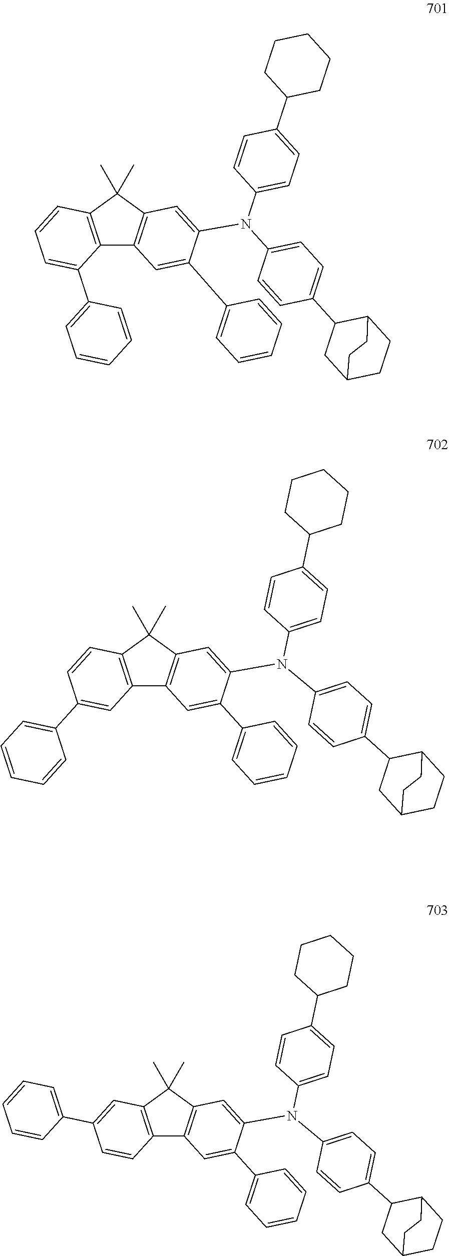 [SMILES: CC1(C)c2cc(N(c3ccc(C4CCCCC4)cc3)c3ccc(C4CC5CCC4CC5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4CC5CCC4CC5)cc3)cc21 ; CC1(C)c2cc(-c3ccccc3)ccc2-c2cc(-c3ccccc3)c(N(c3ccc(C4CCCCC4)cc3)c3ccc(C4CC5CCC4CC5)cc3)cc21] 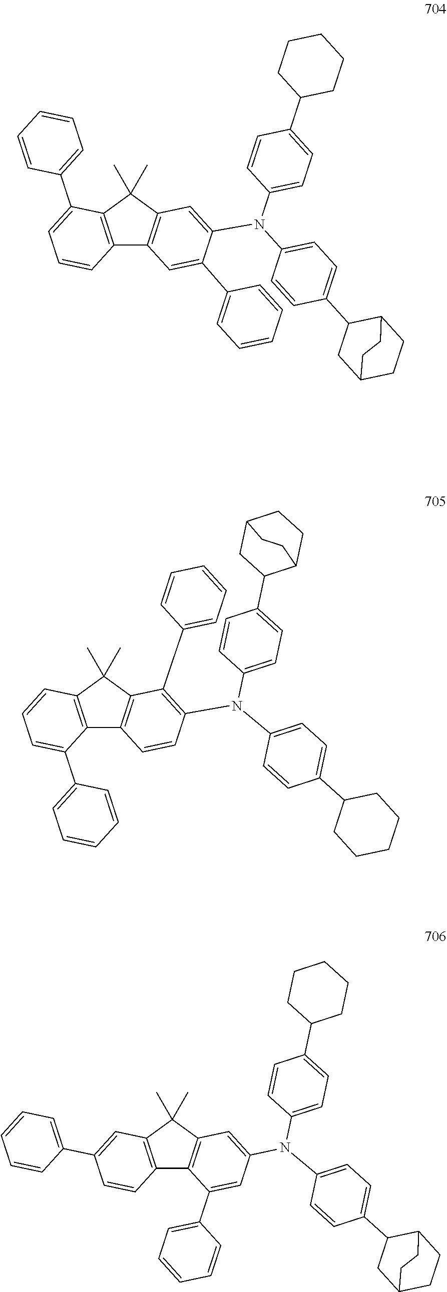 [SMILES: CC1(C)c2cc(N(c3ccc(C4CCCCC4)cc3)c3ccc(C4CC5CCC4CC5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4CC5CCC4CC5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4CC5CCC4CC5)cc3)cc21] 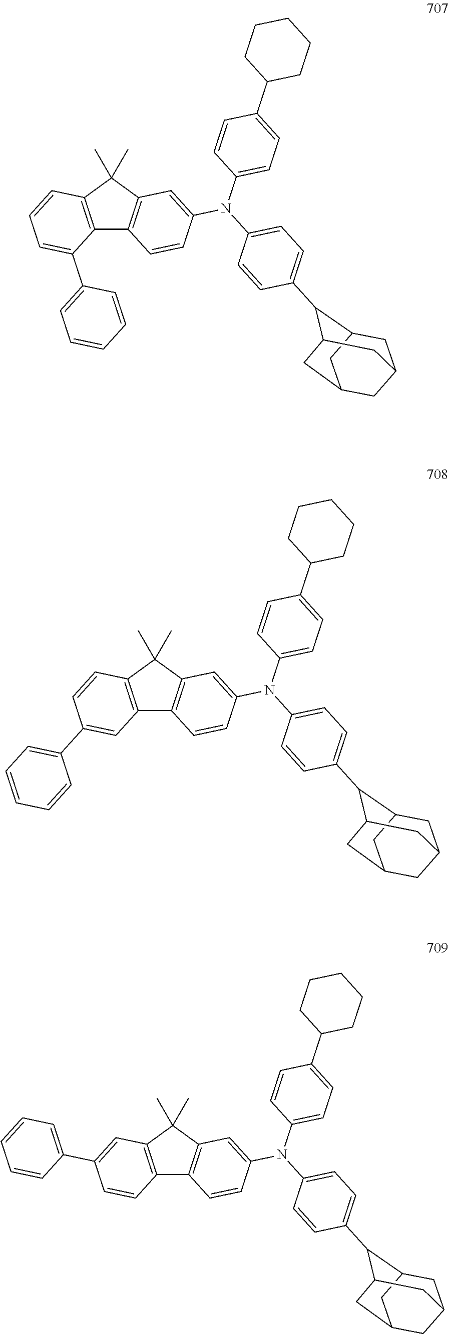 [SMILES: CC1(C)c2cc(N(c3ccc(C4CCCCC4)cc3)c3ccc(C4C5CC6CC(C5)CC4C6)cc3)ccc2-c2c(-c3ccccc3)cccc21 ; CC1(C)c2ccc(-c3ccccc3)cc2-c2ccc(N(c3ccc(C4CCCCC4)cc3)c3ccc(C4C5CC6CC(C5)CC4C6)cc3)cc21 ; CC1(C)c2cc(-c3ccccc3)ccc2-c2ccc(N(c3ccc(C4CCCCC4)cc3)c3ccc(C4C5CC6CC(C5)CC4C6)cc3)cc21] 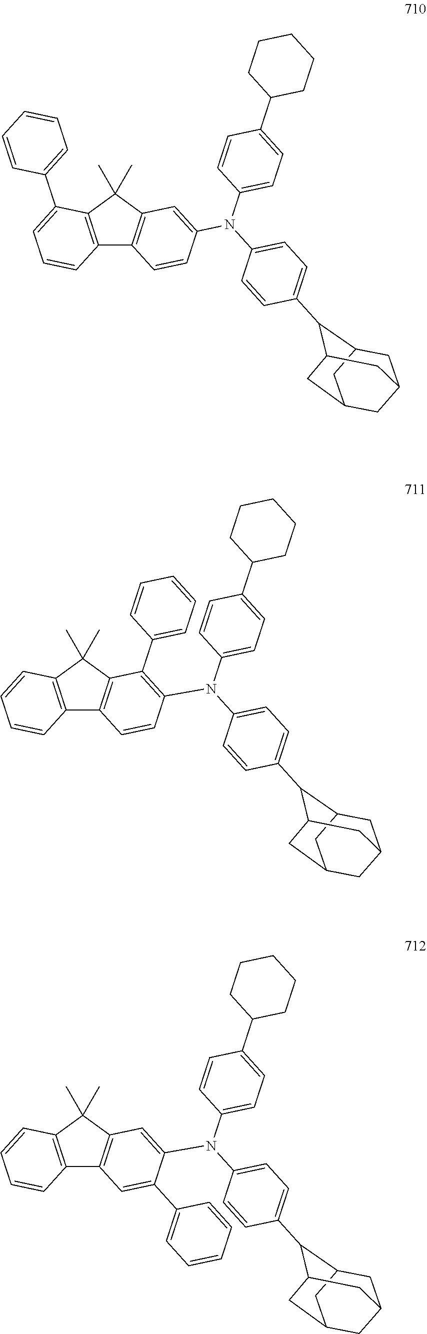 [SMILES: CC1(C)c2cc(N(c3ccc(C4CCCCC4)cc3)c3ccc(C4C5CC6CC(C5)CC4C6)cc3)ccc2-c2cccc(-c3ccccc3)c21 ; CC1(C)c2ccccc2-c2ccc(N(c3ccc(C4CCCCC4)cc3)c3ccc(C4C5CC6CC(C5)CC4C6)cc3)c(-c3ccccc3)c21 ; CC1(C)c2ccccc2-c2cc(-c3ccccc3)c(N(c3ccc(C4CCCCC4)cc3)c3ccc(C4C5CC6CC(C5)CC4C6)cc3)cc21] 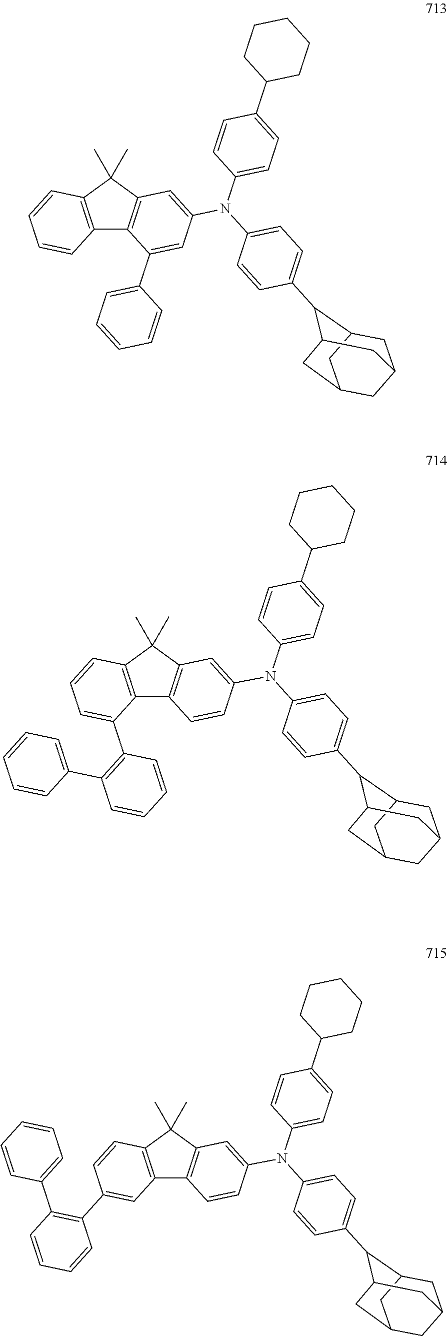 [SMILES: CC1(C)c2ccccc2-c2c(-c3ccccc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C4C5CC6CC(C5)CC4C6)cc3)cc21] 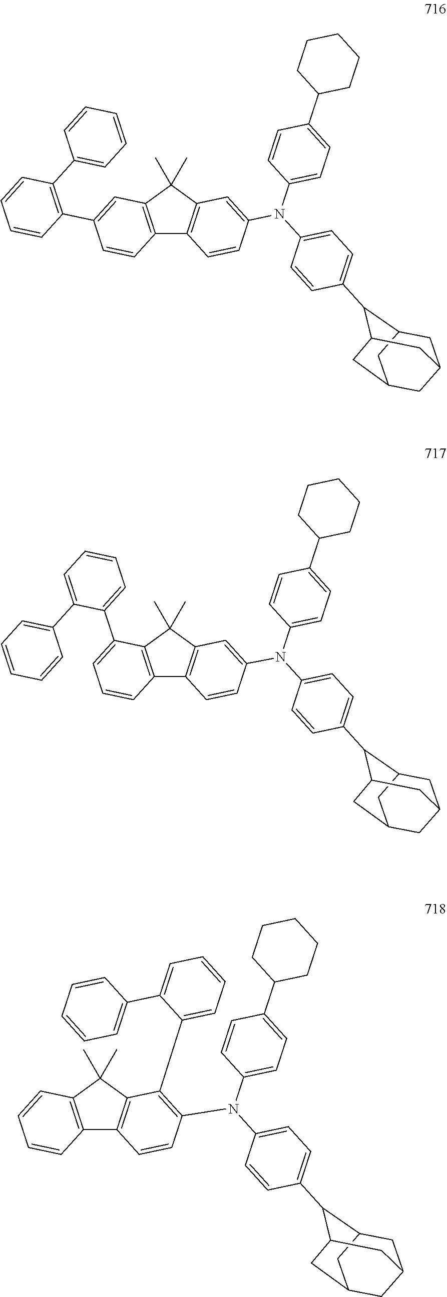 [SMILES: CC1(C)c2cc(-c3ccccc3-c3ccccc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccccc3-c3ccccc3)c21 ; CC1(C)c2ccccc2-c2ccc(N(c3ccc(C4CCCCC4)cc3)c3ccc(C4C5CC6CC(C5)CC4C6)cc3)c(-c3ccccc3-c3ccccc3)c21] 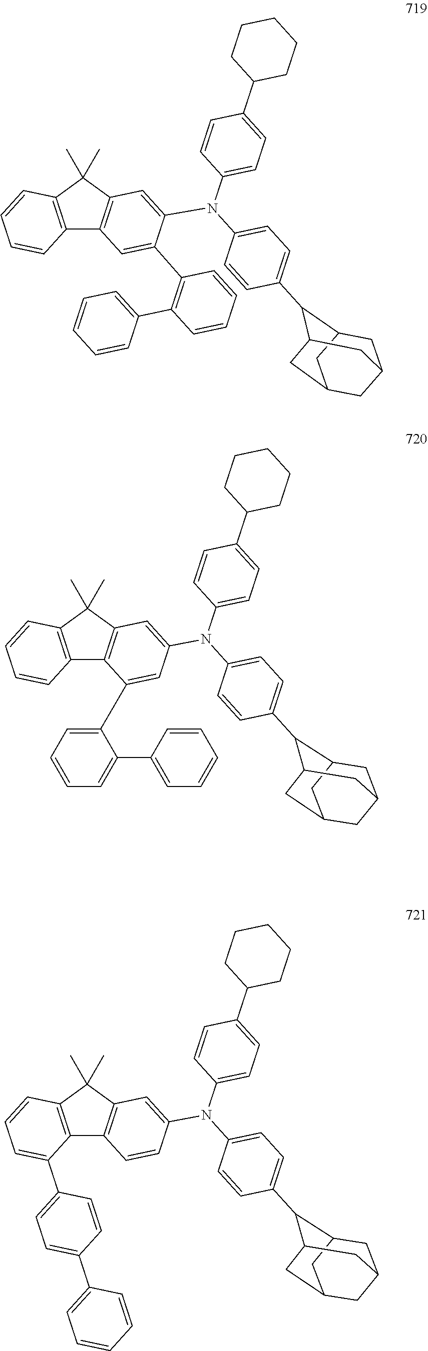 [SMILES: CC1(C)c2ccccc2-c2cc(-c3ccccc3-c3ccccc3)c(N(c3ccc(C4CCCCC4)cc3)c3ccc(C4C5CC6CC(C5)CC4C6)cc3)cc21 ; CC1(C)c2ccccc2-c2c(-c3ccccc3-c3ccccc3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc(-c4ccccc4)cc3)cccc21] 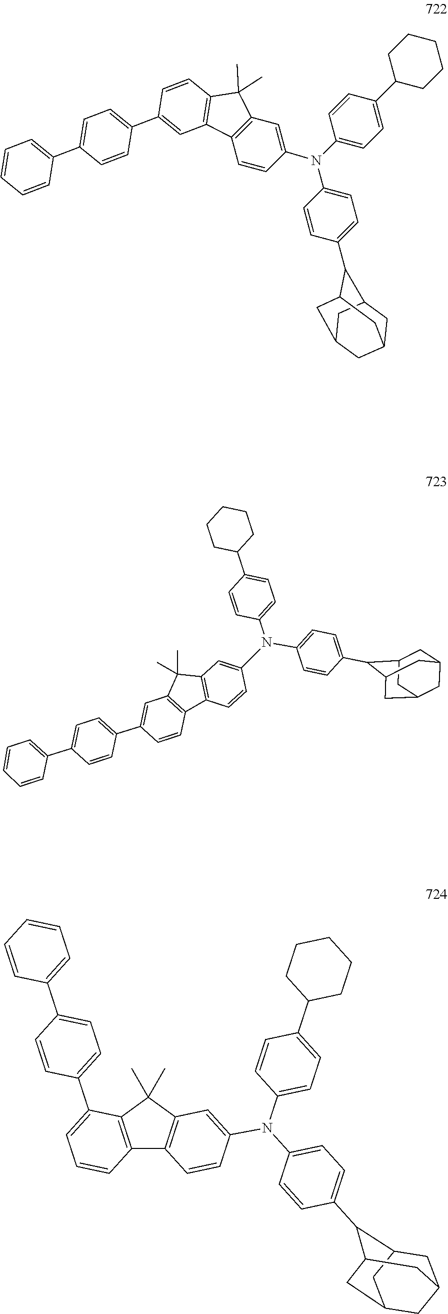 [SMILES: CC1(C)c2ccc(-c3ccc(-c4ccccc4)cc3)cc2-c2ccc(N(c3ccc(C4CCCCC4)cc3)c3ccc(C4C5CC6CC(C5)CC4C6)cc3)cc21 ; CC1(C)c2cc(-c3ccc(-c4ccccc4)cc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc(-c4ccccc4)cc3)c21] 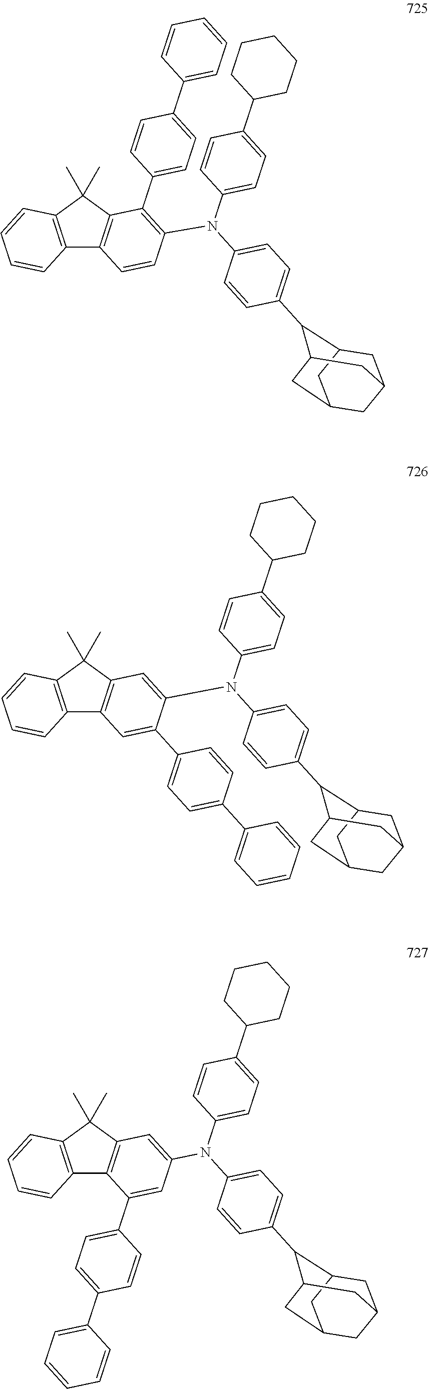 [SMILES: CC1(C)c2ccccc2-c2ccc(N(c3ccc(C4CCCCC4)cc3)c3ccc(C4C5CC6CC(C5)CC4C6)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C4C5CC6CC(C5)CC4C6)cc3)cc21 ; CC1(C)c2ccccc2-c2c(-c3ccc(-c4ccccc4)cc3)cc(N(c3ccc(C4CCCCC4)cc3)c3ccc(C4C5CC6CC(C5)CC4C6)cc3)cc21] 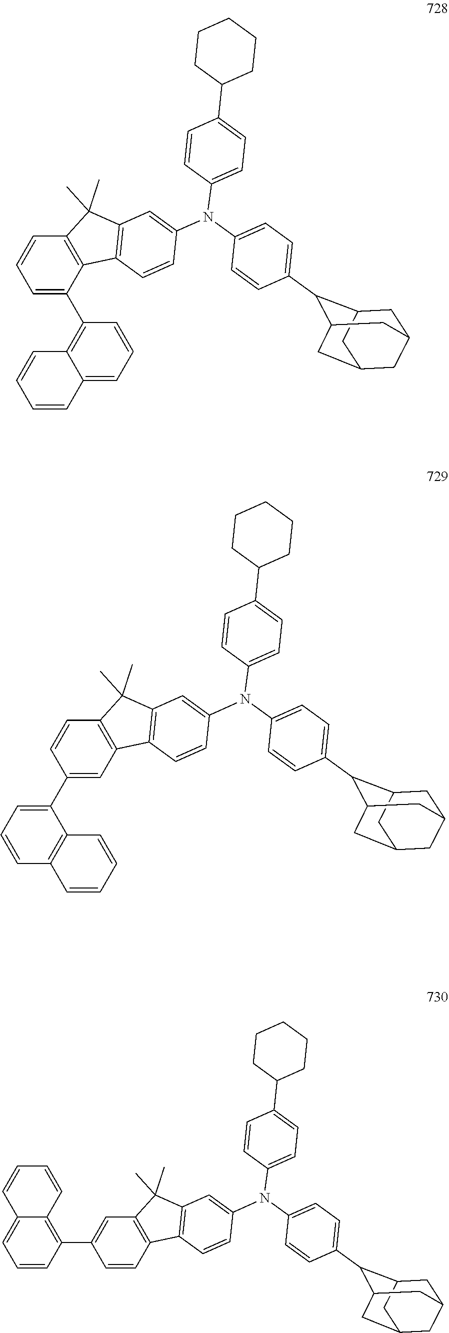 [SMILES: CC1(C)c2cc(N(c3ccc(C4CCCCC4)cc3)c3ccc(C4C5CC6CC(C5)CC4C6)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4C5CC6CC(C5)CC4C6)cc3)cc21 ; CC1(C)c2cc(-c3cccc4ccccc34)ccc2-c2ccc(N(c3ccc(C4CCCCC4)cc3)c3ccc(C4C5CC6CC(C5)CC4C6)cc3)cc21] 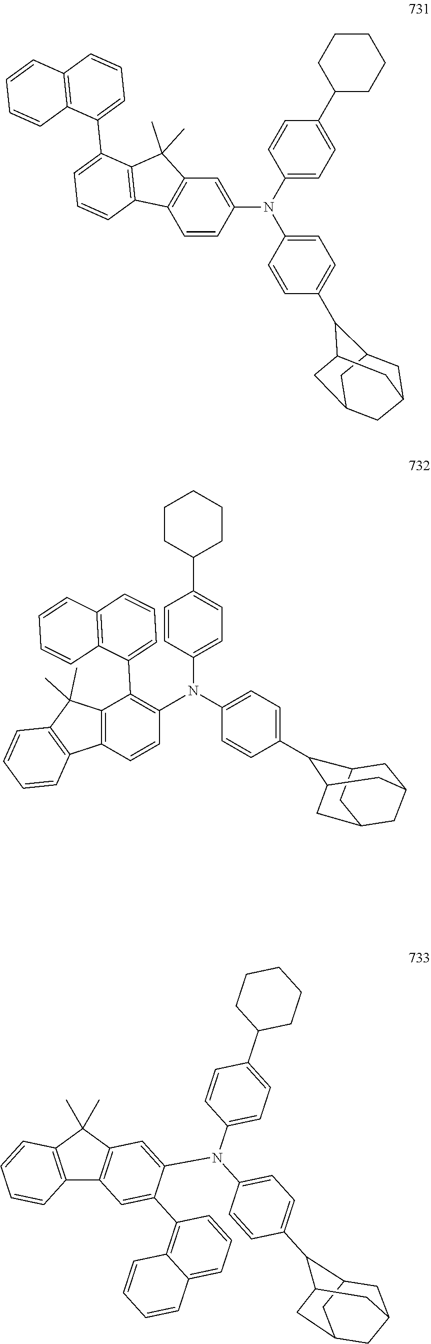 [SMILES: CC1(C)c2cc(N(c3ccc(C4CCCCC4)cc3)c3ccc(C4C5CC6CC(C5)CC4C6)cc3)ccc2-c2cccc(-c3cccc4ccccc34)c21 ; CC1(C)c2ccccc2-c2ccc(N(c3ccc(C4CCCCC4)cc3)c3ccc(C4C5CC6CC(C5)CC4C6)cc3)c(-c3cccc4ccccc34)c21 ; CC1(C)c2ccccc2-c2cc(-c3cccc4ccccc34)c(N(c3ccc(C4CCCCC4)cc3)c3ccc(C4C5CC6CC(C5)CC4C6)cc3)cc21] 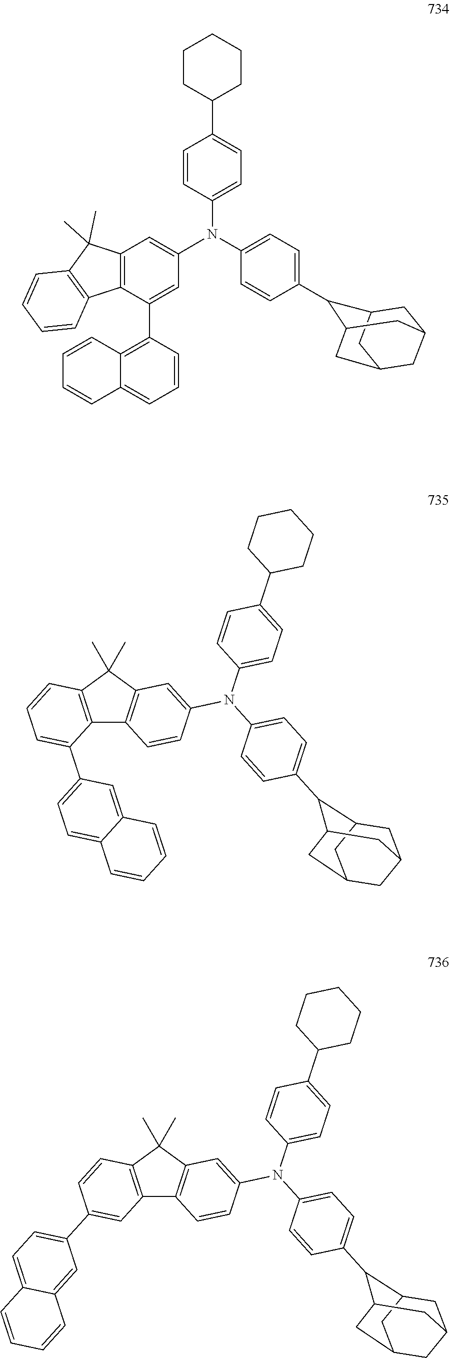 [SMILES: CC1(C)c2ccccc2-c2c(-c3cccc4ccccc34)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4C5CC6CC(C5)CC4C6)cc3)cc21] 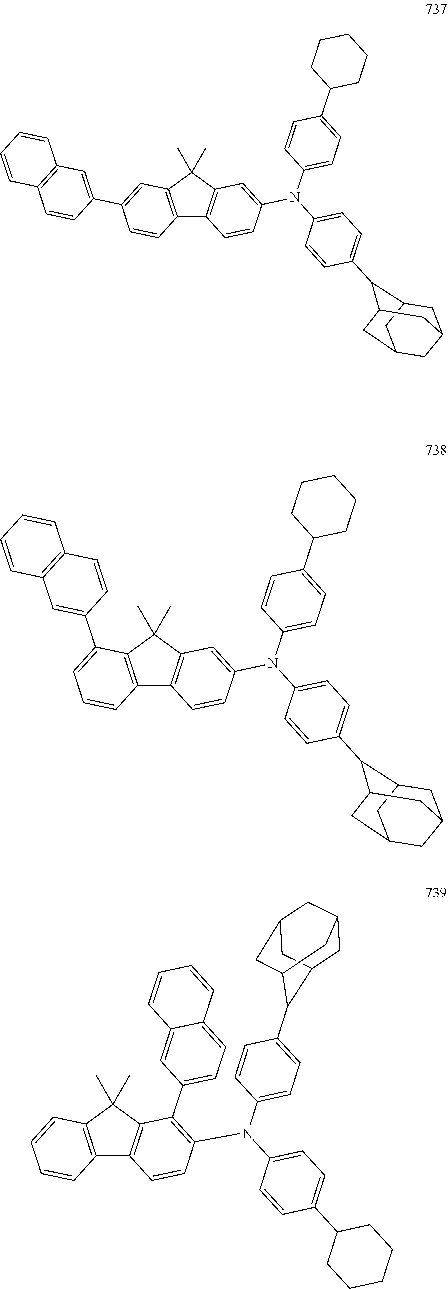 [SMILES: CC1(C)c2cc(-c3ccc4ccccc4c3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc4ccccc4c3)c21 ; CC1(C)c2ccccc2-c2ccc(N(c3ccc(C4CCCCC4)cc3)c3ccc(C4C5CC6CC(C5)CC4C6)cc3)c(-c3ccc4ccccc4c3)c21] 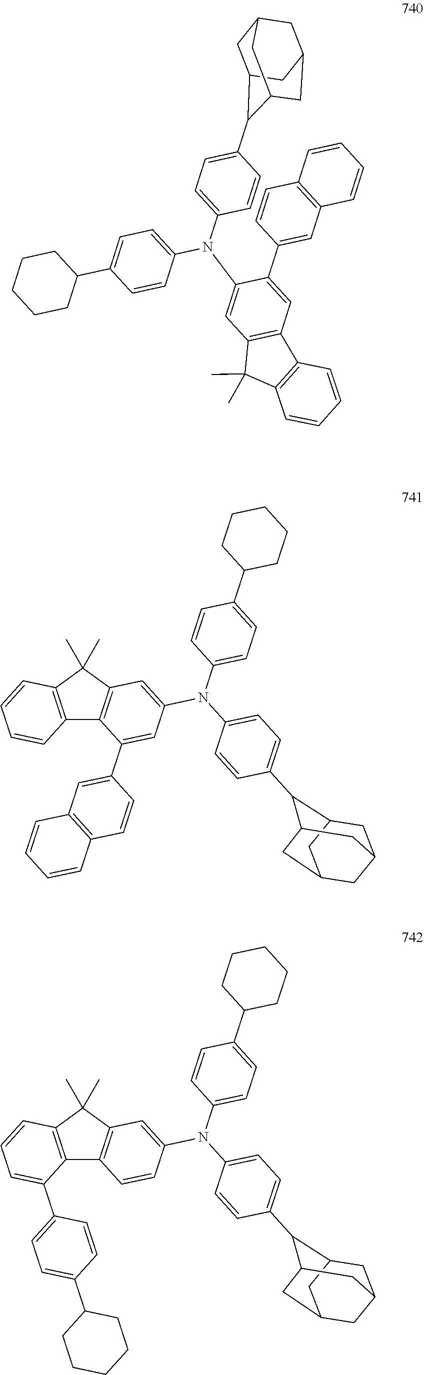 [SMILES: CC1(C)c2ccccc2-c2cc(-c3ccc4ccccc4c3)c(N(c3ccc(C4CCCCC4)cc3)c3ccc(C4C5CC6CC(C5)CC4C6)cc3)cc21 ; CC1(C)c2ccccc2-c2c(-c3ccc4ccccc4c3)cc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2c(-c3ccc(C4CCCCC4)cc3)cccc21] 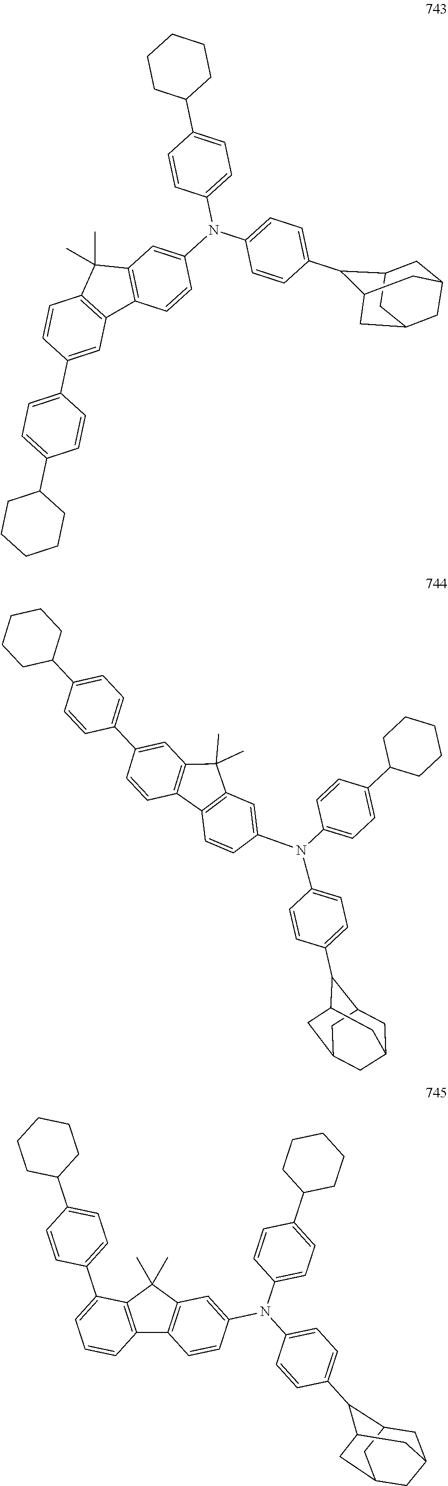 [SMILES: CC1(C)c2ccc(-c3ccc(C4CCCCC4)cc3)cc2-c2ccc(N(c3ccc(C4CCCCC4)cc3)c3ccc(C4C5CC6CC(C5)CC4C6)cc3)cc21 ; CC1(C)c2cc(-c3ccc(C4CCCCC4)cc3)ccc2-c2ccc(N(c3ccc(C4CCCCC4)cc3)c3ccc(C4C5CC6CC(C5)CC4C6)cc3)cc21 ; CC1(C)c2cc(N(c3ccc(C4CCCCC4)cc3)c3ccc(C4C5CC6CC(C5)CC4C6)cc3)ccc2-c2cccc(-c3ccc(C4CCCCC4)cc3)c21] 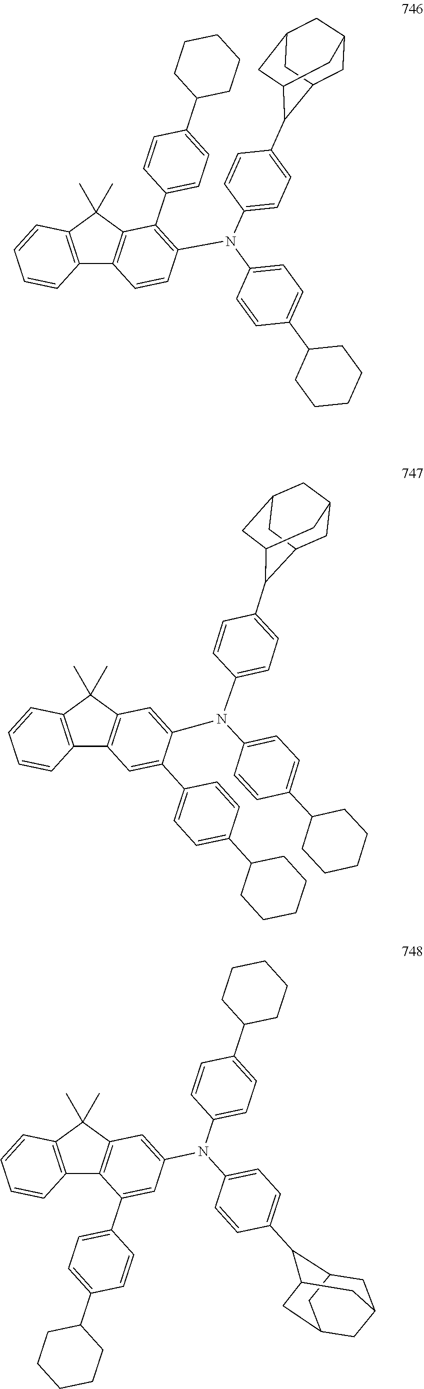 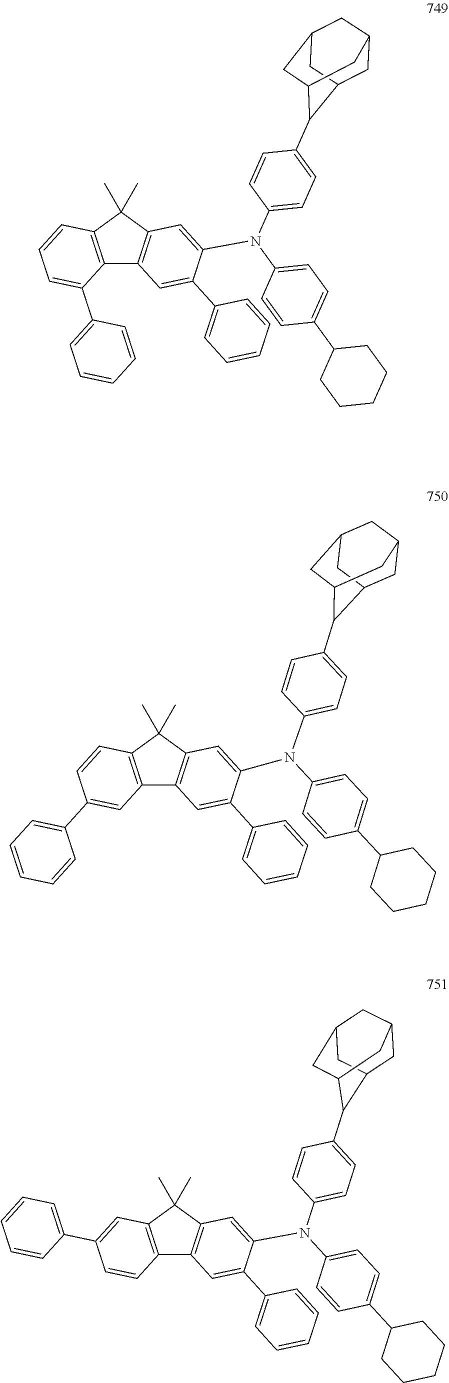 [SMILES: CC1(C)c2cc(N(c3ccc(C4CCCCC4)cc3)c3ccc(C4C5CC6CC(C5)CC4C6)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C4C5CC6CC(C5)CC4C6)cc3)cc21 ; CC1(C)c2cc(-c3ccccc3)ccc2-c2cc(-c3ccccc3)c(N(c3ccc(C4CCCCC4)cc3)c3ccc(C4C5CC6CC(C5)CC4C6)cc3)cc21] 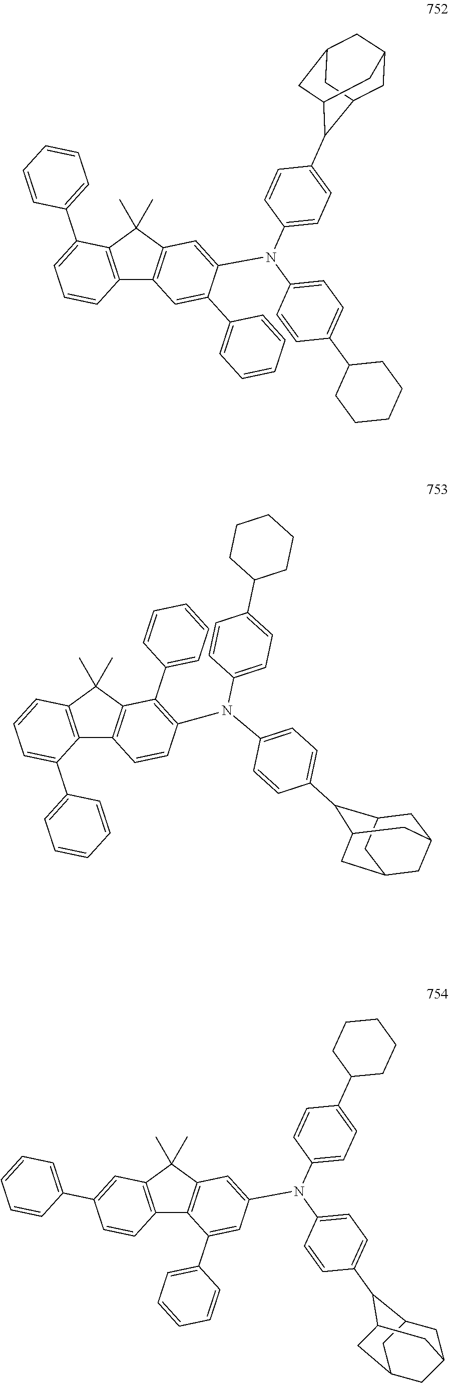 [SMILES: CC1(C)c2cc(N(c3ccc(C4CCCCC4)cc3)c3ccc(C4C5CC6CC(C5)CC4C6)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C4C5CC6CC(C5)CC4C6)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C4C5CC6CC(C5)CC4C6)cc3)cc21] 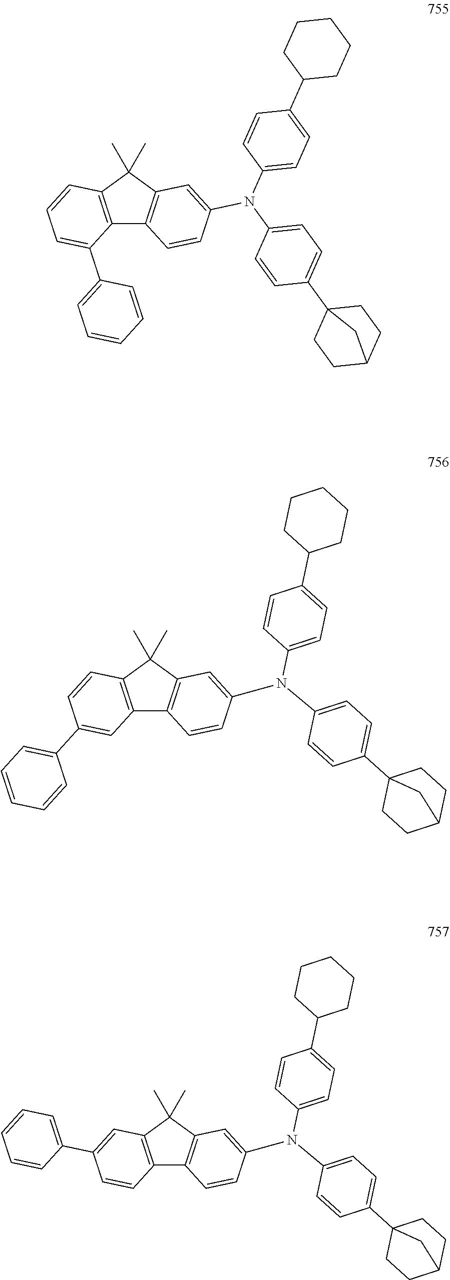 [SMILES: CC1(C)c2cc(N(c3ccc(C4CCCCC4)cc3)c3ccc(C45CCC(CC4)C5)cc3)ccc2-c2c(-c3ccccc3)cccc21 ; CC1(C)c2ccc(-c3ccccc3)cc2-c2ccc(N(c3ccc(C4CCCCC4)cc3)c3ccc(C45CCC(CC4)C5)cc3)cc21 ; CC1(C)c2cc(-c3ccccc3)ccc2-c2ccc(N(c3ccc(C4CCCCC4)cc3)c3ccc(C45CCC(CC4)C5)cc3)cc21] 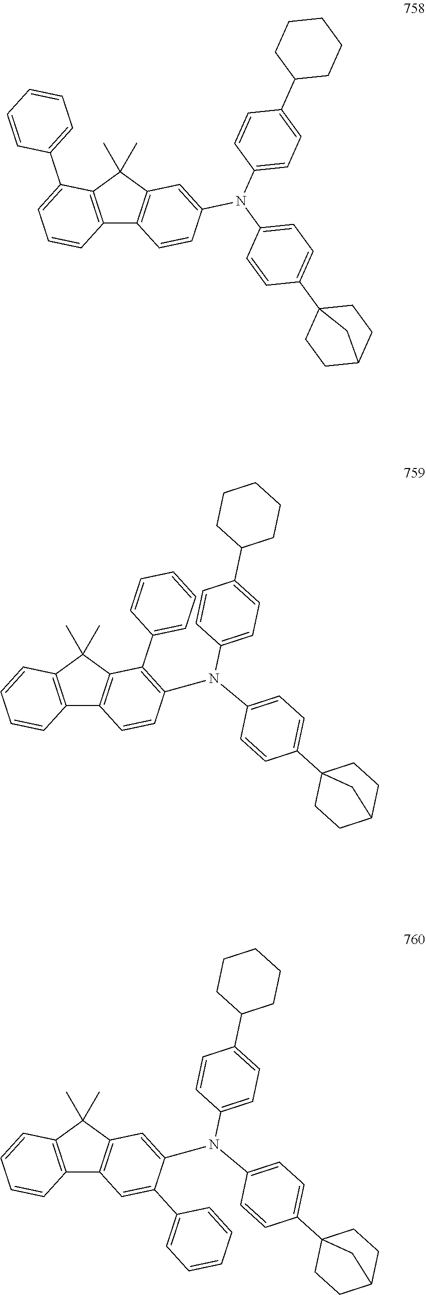 [SMILES: CC1(C)c2cc(N(c3ccc(C4CCCCC4)cc3)c3ccc(C45CCC(CC4)C5)cc3)ccc2-c2cccc(-c3ccccc3)c21 ; CC1(C)c2ccccc2-c2ccc(N(c3ccc(C4CCCCC4)cc3)c3ccc(C45CCC(CC4)C5)cc3)c(-c3ccccc3)c21 ; CC1(C)c2ccccc2-c2cc(-c3ccccc3)c(N(c3ccc(C4CCCCC4)cc3)c3ccc(C45CCC(CC4)C5)cc3)cc21] 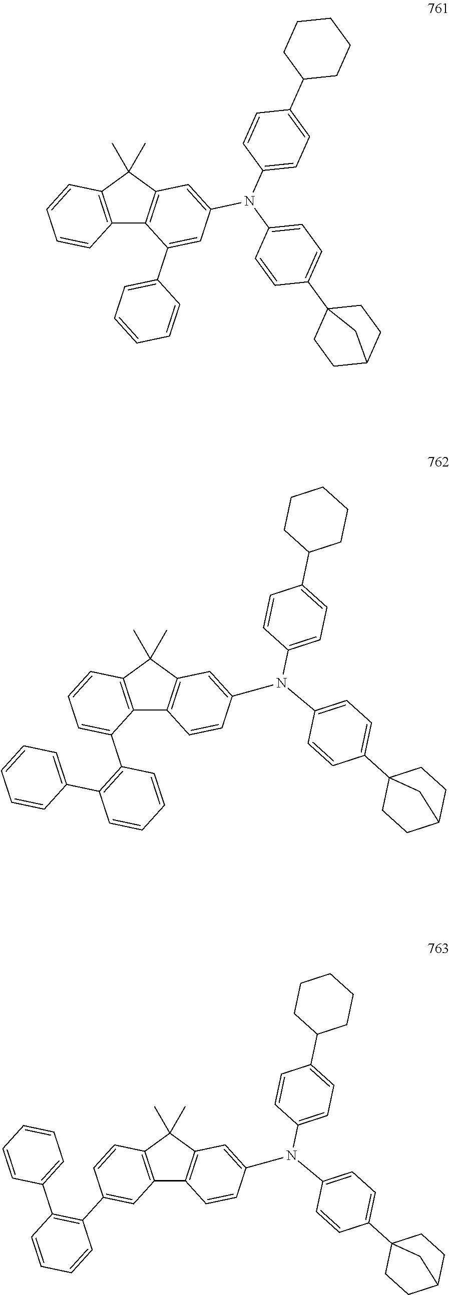 [SMILES: CC1(C)c2ccccc2-c2c(-c3ccccc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C45CCC(CC4)C5)cc3)cc21] 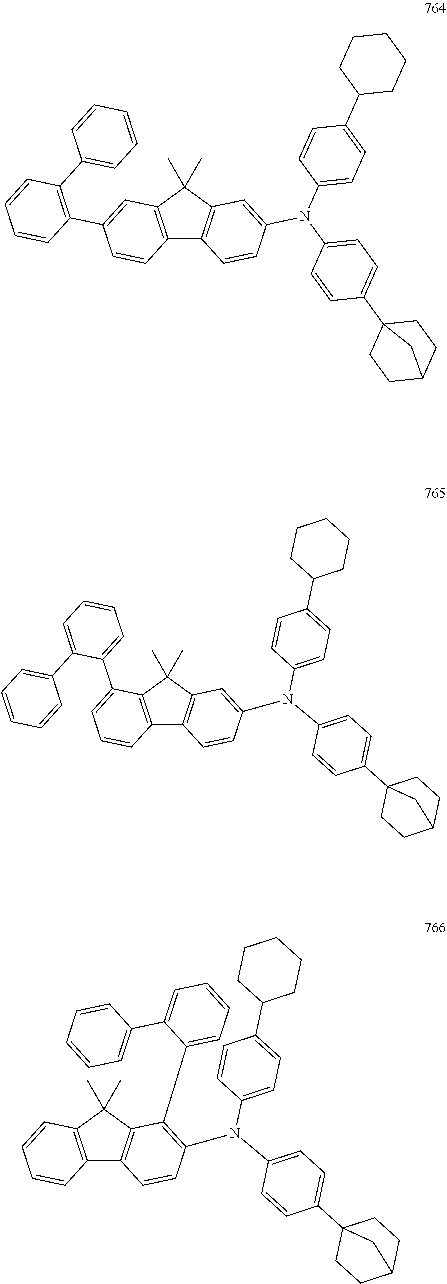 [SMILES: CC1(C)c2cc(-c3ccccc3-c3ccccc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccccc3-c3ccccc3)c21 ; CC1(C)c2ccccc2-c2ccc(N(c3ccc(C4CCCCC4)cc3)c3ccc(C45CCC(CC4)C5)cc3)c(-c3ccccc3-c3ccccc3)c21] 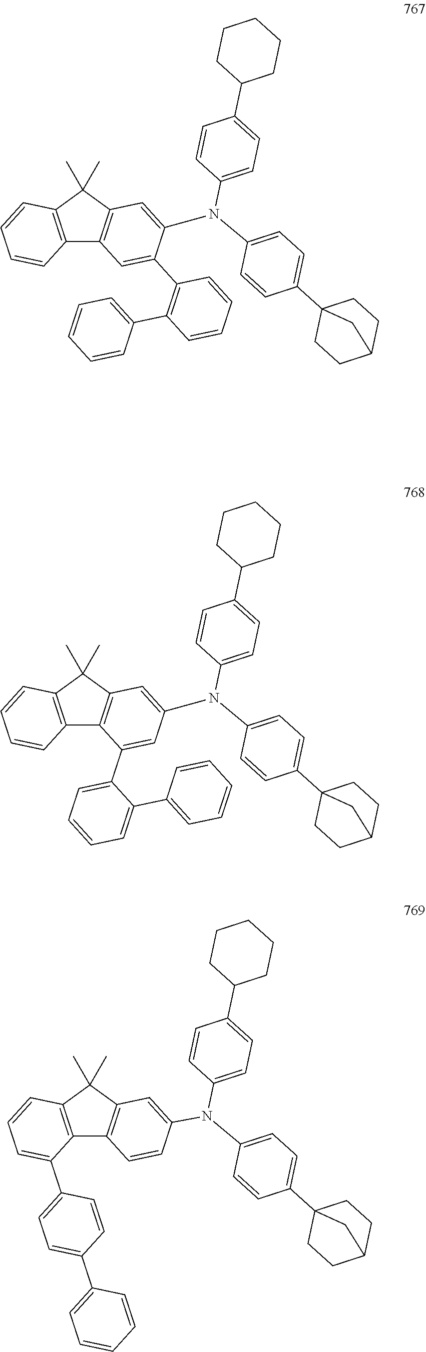 [SMILES: CC1(C)c2ccccc2-c2cc(-c3ccccc3-c3ccccc3)c(N(c3ccc(C4CCCCC4)cc3)c3ccc(C45CCC(CC4)C5)cc3)cc21 ; CC1(C)c2ccccc2-c2c(-c3ccccc3-c3ccccc3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc(-c4ccccc4)cc3)cccc21] 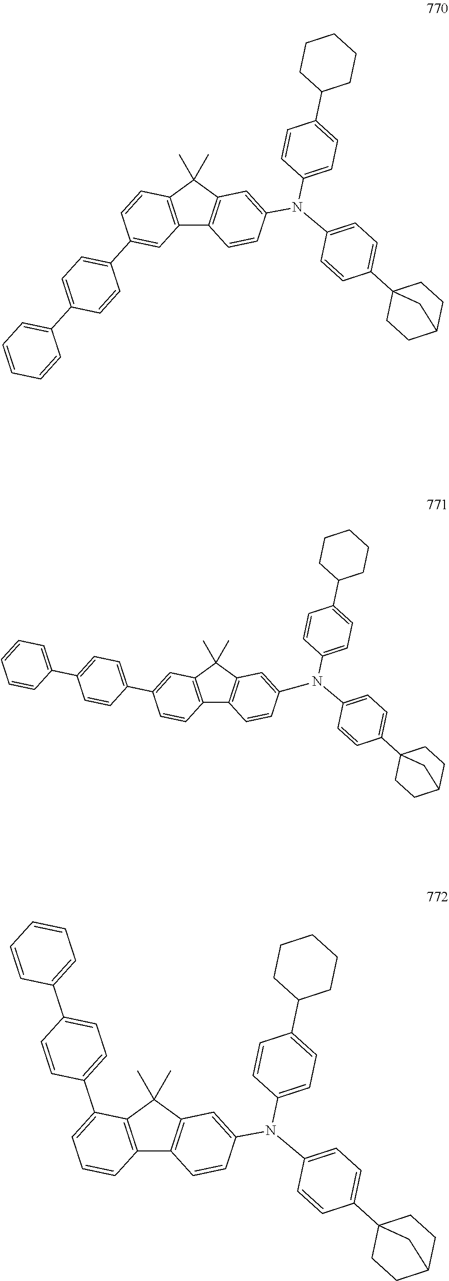 [SMILES: CC1(C)c2ccc(-c3ccc(-c4ccccc4)cc3)cc2-c2ccc(N(c3ccc(C4CCCCC4)cc3)c3ccc(C45CCC(CC4)C5)cc3)cc21 ; CC1(C)c2cc(-c3ccc(-c4ccccc4)cc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc(-c4ccccc4)cc3)c21] 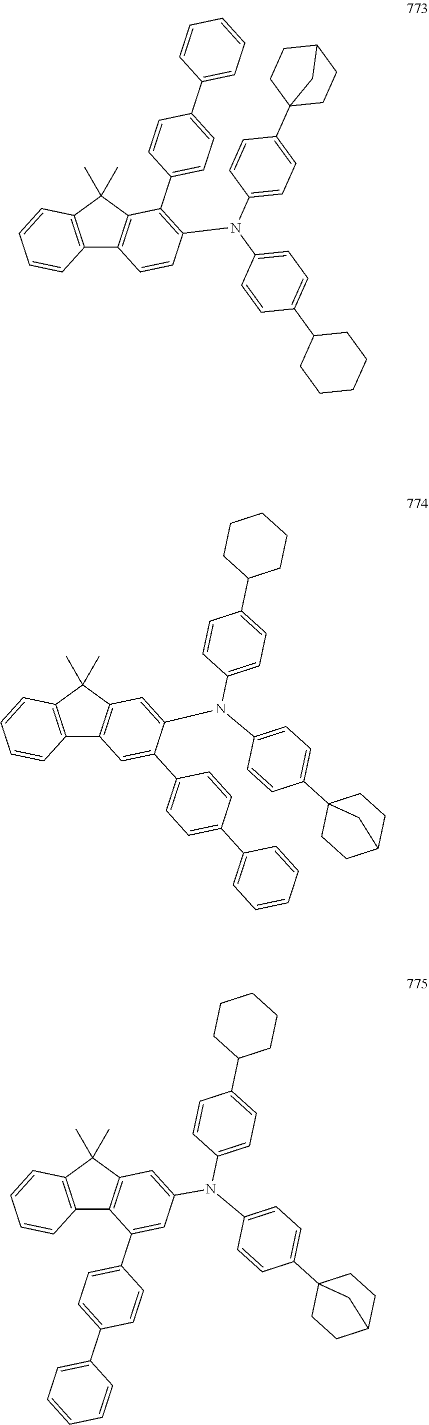 [SMILES: CC1(C)c2ccccc2-c2ccc(N(c3ccc(C4CCCCC4)cc3)c3ccc(C45CCC(CC4)C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C45CCC(CC4)C5)cc3)cc21 ; CC1(C)c2ccccc2-c2c(-c3ccc(-c4ccccc4)cc3)cc(N(c3ccc(C4CCCCC4)cc3)c3ccc(C45CCC(CC4)C5)cc3)cc21] 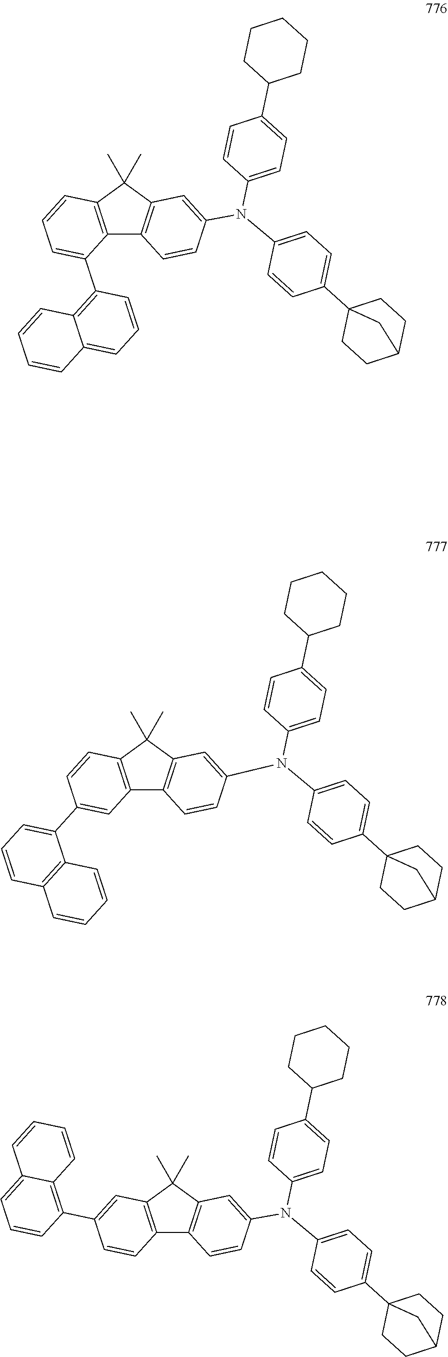 [SMILES: CC1(C)c2cc(N(c3ccc(C4CCCCC4)cc3)c3ccc(C45CCC(CC4)C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C45CCC(CC4)C5)cc3)cc21 ; CC1(C)c2cc(-c3cccc4ccccc34)ccc2-c2ccc(N(c3ccc(C4CCCCC4)cc3)c3ccc(C45CCC(CC4)C5)cc3)cc21] 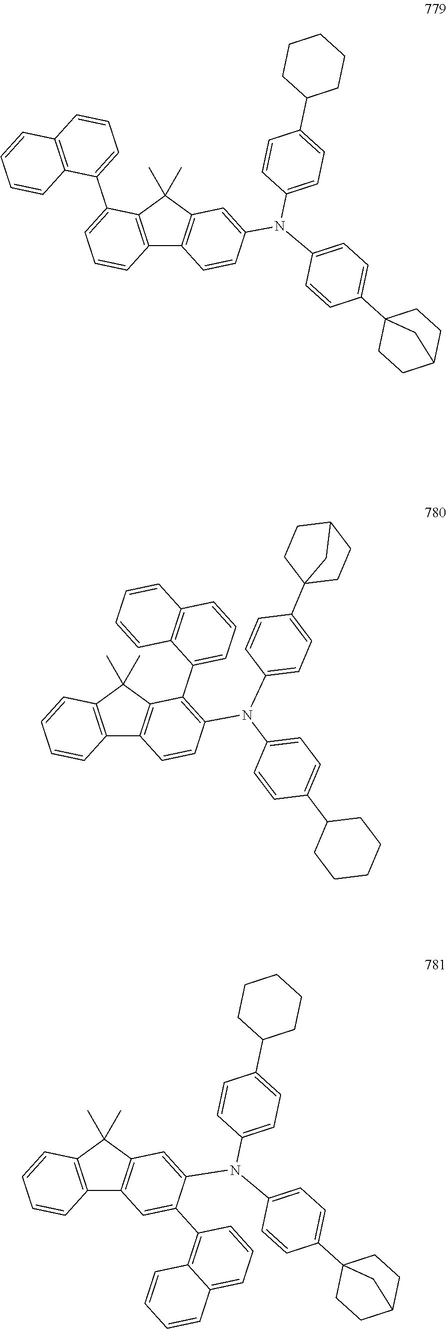 [SMILES: CC1(C)c2cc(N(c3ccc(C4CCCCC4)cc3)c3ccc(C45CCC(CC4)C5)cc3)ccc2-c2cccc(-c3cccc4ccccc34)c21 ; CC1(C)c2ccccc2-c2ccc(N(c3ccc(C4CCCCC4)cc3)c3ccc(C45CCC(CC4)C5)cc3)c(-c3cccc4ccccc34)c21 ; CC1(C)c2ccccc2-c2cc(-c3cccc4ccccc34)c(N(c3ccc(C4CCCCC4)cc3)c3ccc(C45CCC(CC4)C5)cc3)cc21] 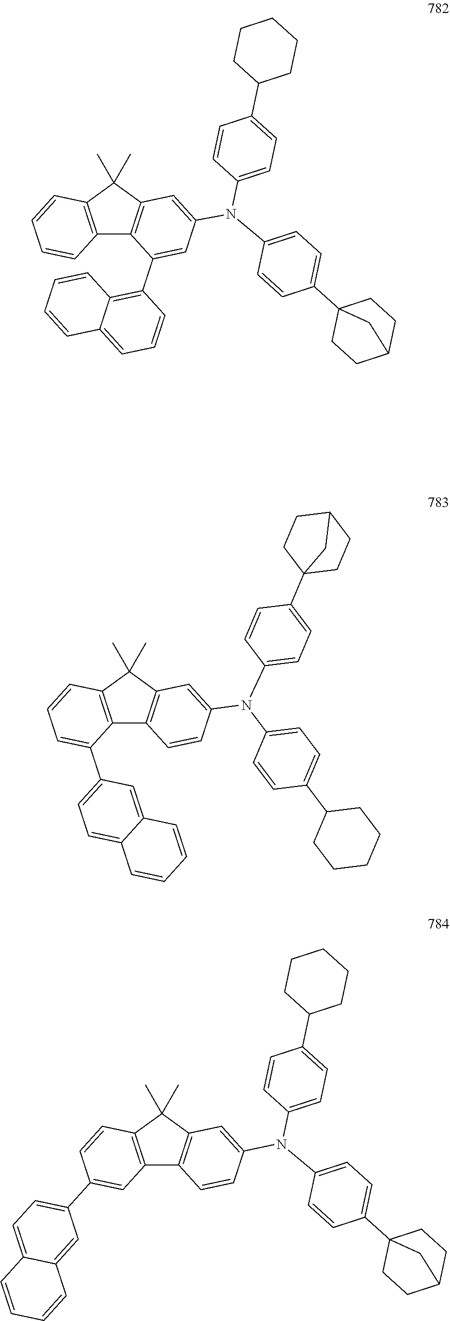 [SMILES: CC1(C)c2ccccc2-c2c(-c3cccc4ccccc34)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C45CCC(CC4)C5)cc3)cc21] 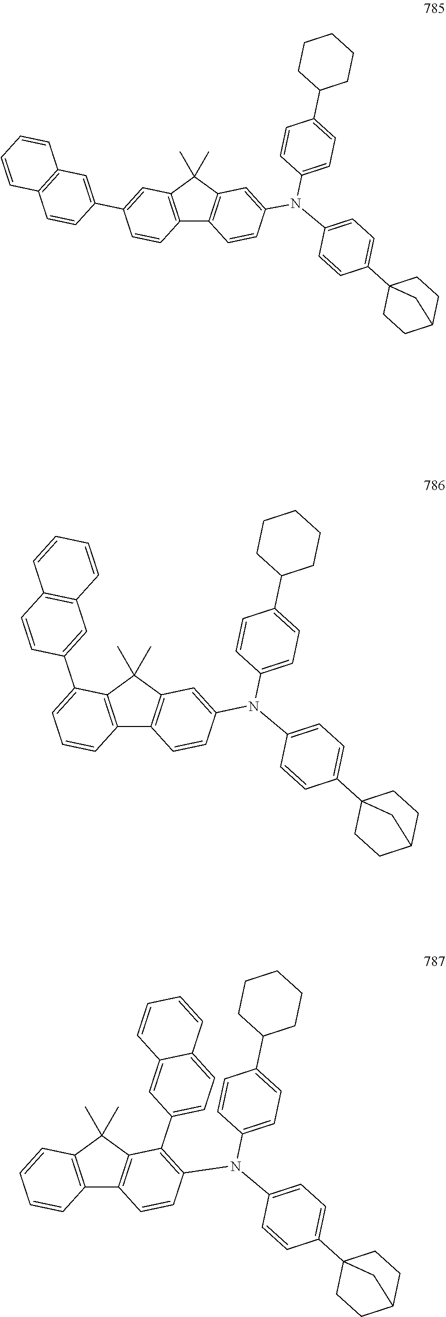 [SMILES: CC1(C)c2cc(-c3ccc4ccccc4c3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc4ccccc4c3)c21 ; CC1(C)c2ccccc2-c2ccc(N(c3ccc(C4CCCCC4)cc3)c3ccc(C45CCC(CC4)C5)cc3)c(-c3ccc4ccccc4c3)c21] 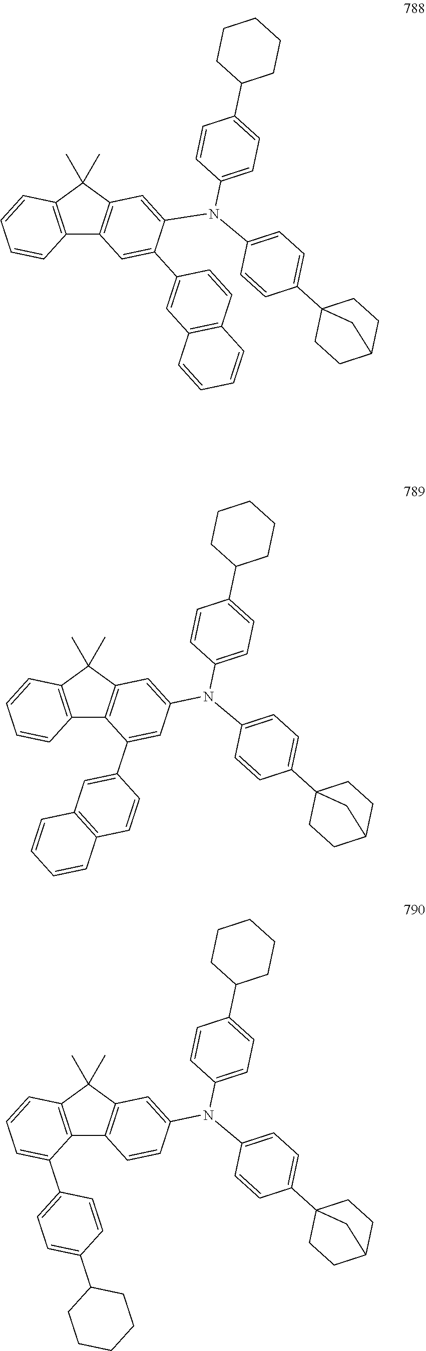 [SMILES: CC1(C)c2ccccc2-c2cc(-c3ccc4ccccc4c3)c(N(c3ccc(C4CCCCC4)cc3)c3ccc(C45CCC(CC4)C5)cc3)cc21 ; CC1(C)c2ccccc2-c2c(-c3ccc4ccccc4c3)cc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2c(-c3ccc(C4CCCCC4)cc3)cccc21] 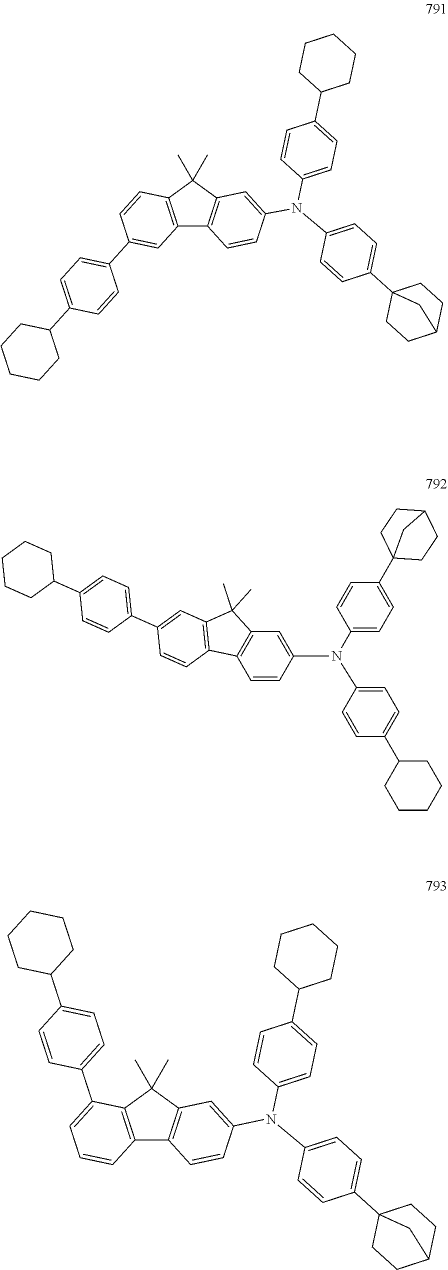 [SMILES: CC1(C)c2ccc(-c3ccc(C4CCCCC4)cc3)cc2-c2ccc(N(c3ccc(C4CCCCC4)cc3)c3ccc(C45CCC(CC4)C5)cc3)cc21 ; CC1(C)c2cc(-c3ccc(C4CCCCC4)cc3)ccc2-c2ccc(N(c3ccc(C4CCCCC4)cc3)c3ccc(C45CCC(CC4)C5)cc3)cc21 ; CC1(C)c2cc(N(c3ccc(C4CCCCC4)cc3)c3ccc(C45CCC(CC4)C5)cc3)ccc2-c2cccc(-c3ccc(C4CCCCC4)cc3)c21] 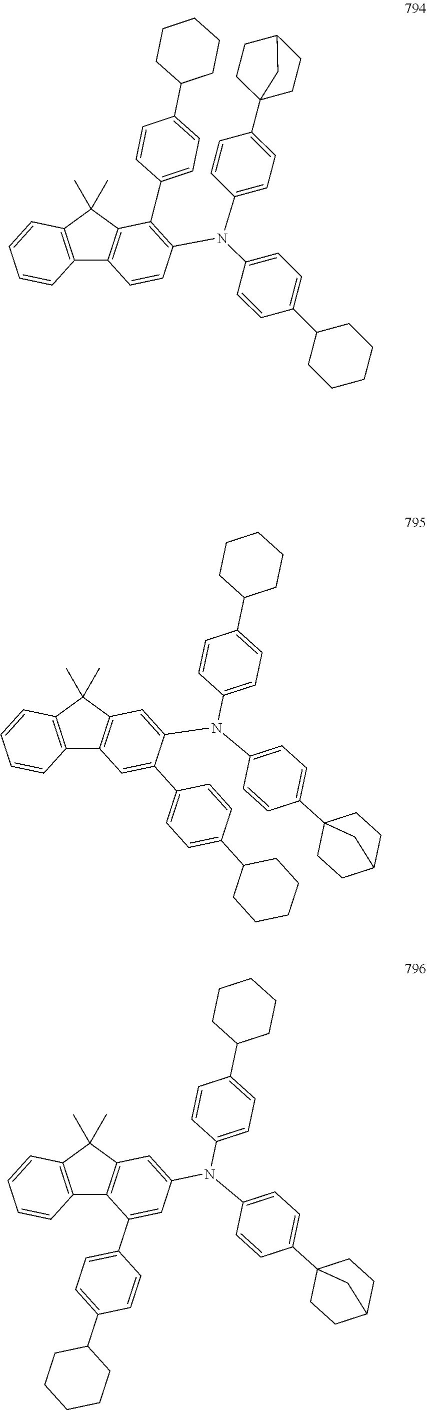 [SMILES: CC1(C)c2ccccc2-c2ccc(N(c3ccc(C4CCCCC4)cc3)c3ccc(C45CCC(CC4)C5)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C45CCC(CC4)C5)cc3)cc21 ; CC1(C)c2ccccc2-c2c(-c3ccc(C4CCCCC4)cc3)cc(N(c3ccc(C4CCCCC4)cc3)c3ccc(C45CCC(CC4)C5)cc3)cc21] 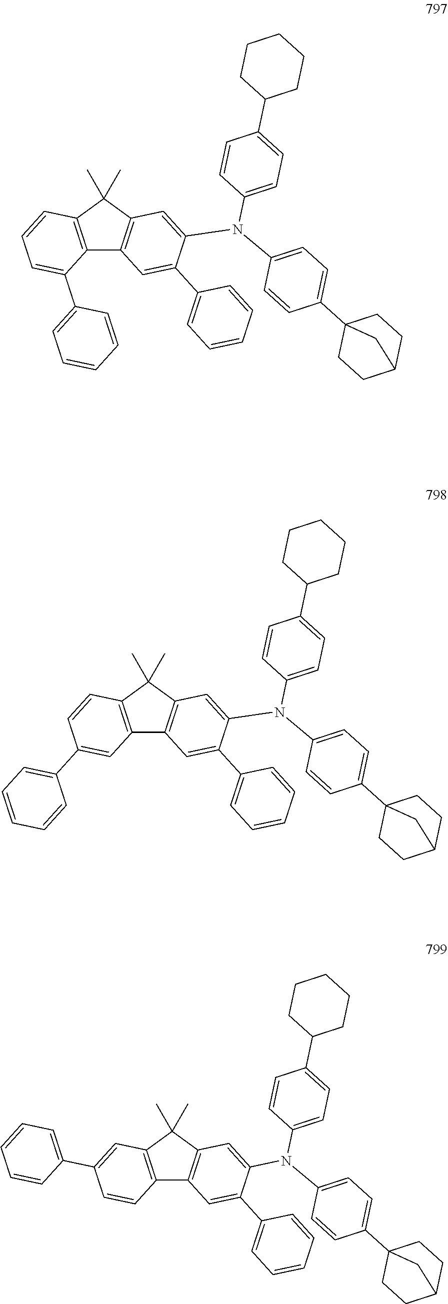 [SMILES: CC1(C)c2cc(N(c3ccc(C4CCCCC4)cc3)c3ccc(C45CCC(CC4)C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C45CCC(CC4)C5)cc3)cc21 ; CC1(C)c2cc(-c3ccccc3)ccc2-c2cc(-c3ccccc3)c(N(c3ccc(C4CCCCC4)cc3)c3ccc(C45CCC(CC4)C5)cc3)cc21] 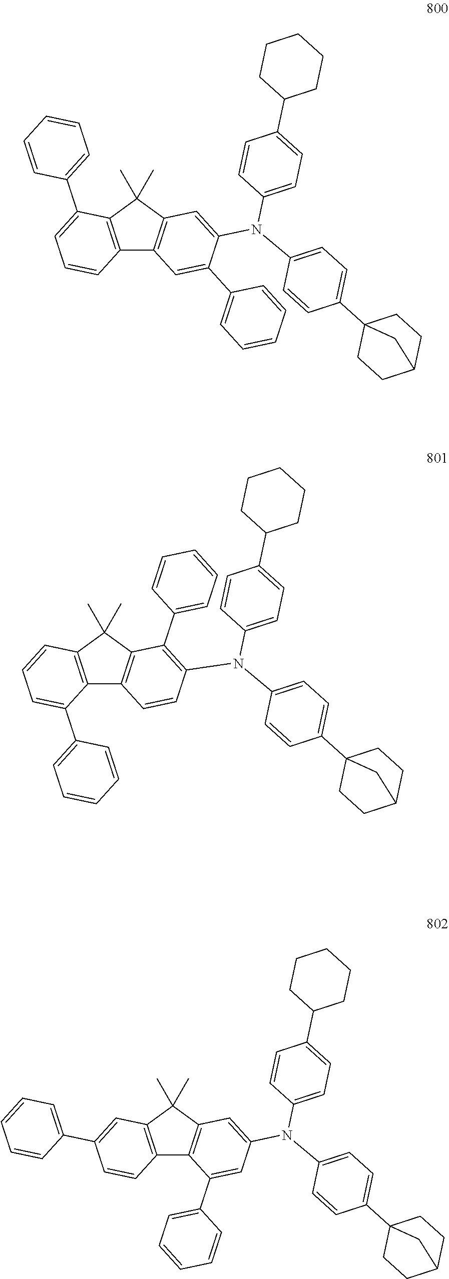 [SMILES: CC1(C)c2cc(N(c3ccc(C4CCCCC4)cc3)c3ccc(C45CCC(CC4)C5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C45CCC(CC4)C5)cc3)c(-c3ccccc3)c21 ; CC1(C)c2cc(-c3ccccc3)ccc2-c2c(-c3ccccc3)cc(N(c3ccc(C4CCCCC4)cc3)c3ccc(C45CCC(CC4)C5)cc3)cc21] 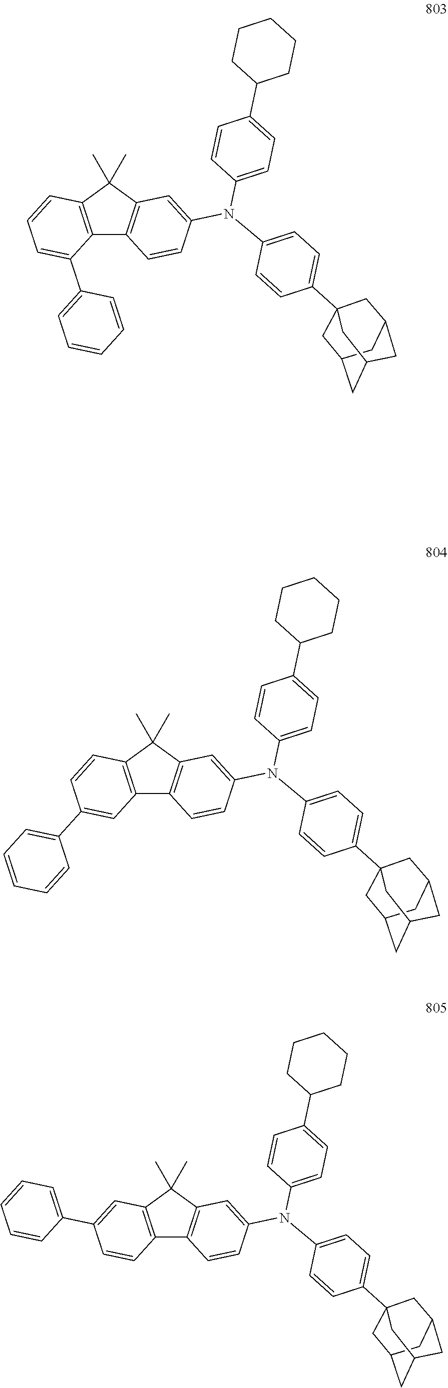 [SMILES: CC1(C)c2cc(N(c3ccc(C4CCCCC4)cc3)c3ccc(C45CC6CC(CC(C6)C4)C5)cc3)ccc2-c2c(-c3ccccc3)cccc21 ; CC1(C)c2ccc(-c3ccccc3)cc2-c2ccc(N(c3ccc(C4CCCCC4)cc3)c3ccc(C45CC6CC(CC(C6)C4)C5)cc3)cc21 ; CC1(C)c2cc(-c3ccccc3)ccc2-c2ccc(N(c3ccc(C4CCCCC4)cc3)c3ccc(C45CC6CC(CC(C6)C4)C5)cc3)cc21] 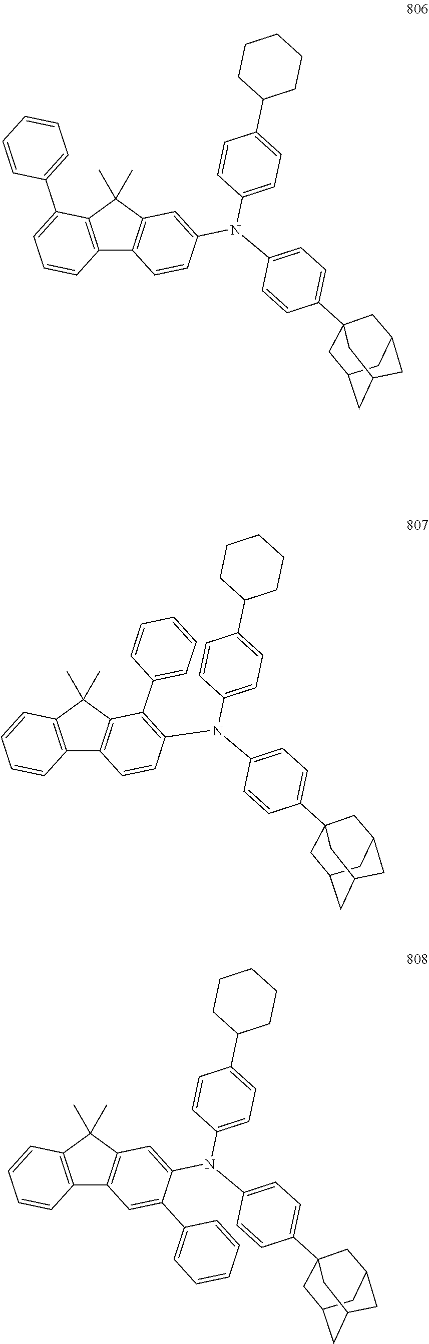 [SMILES: CC1(C)c2cc(N(c3ccc(C4CCCCC4)cc3)c3ccc(C45CC6CC(CC(C6)C4)C5)cc3)ccc2-c2cccc(-c3ccccc3)c21 ; CC1(C)c2ccccc2-c2ccc(N(c3ccc(C4CCCCC4)cc3)c3ccc(C45CC6CC(CC(C6)C4)C5)cc3)c(-c3ccccc3)c21 ; CC1(C)c2ccccc2-c2cc(-c3ccccc3)c(N(c3ccc(C4CCCCC4)cc3)c3ccc(C45CC6CC(CC(C6)C4)C5)cc3)cc21] 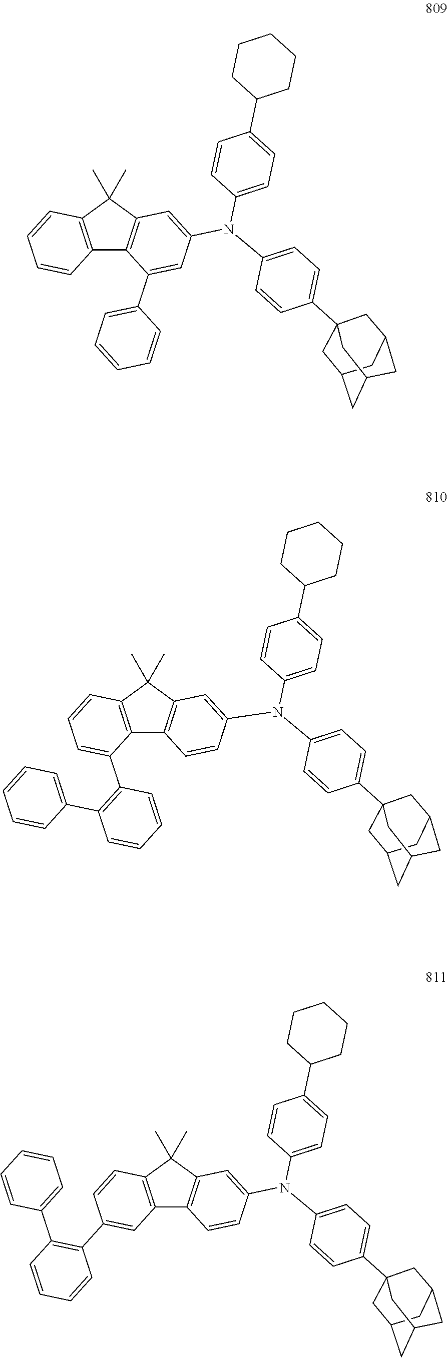 [SMILES: CC1(C)c2ccccc2-c2c(-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccccc3-c3ccccc3)cccc21 ; CC1(C)c2ccc(-c3ccccc3-c3ccccc3)cc2-c2ccc(N(c3ccc(C4CCCCC4)cc3)c3ccc(C45CC6CC(CC(C6)C4)C5)cc3)cc21] 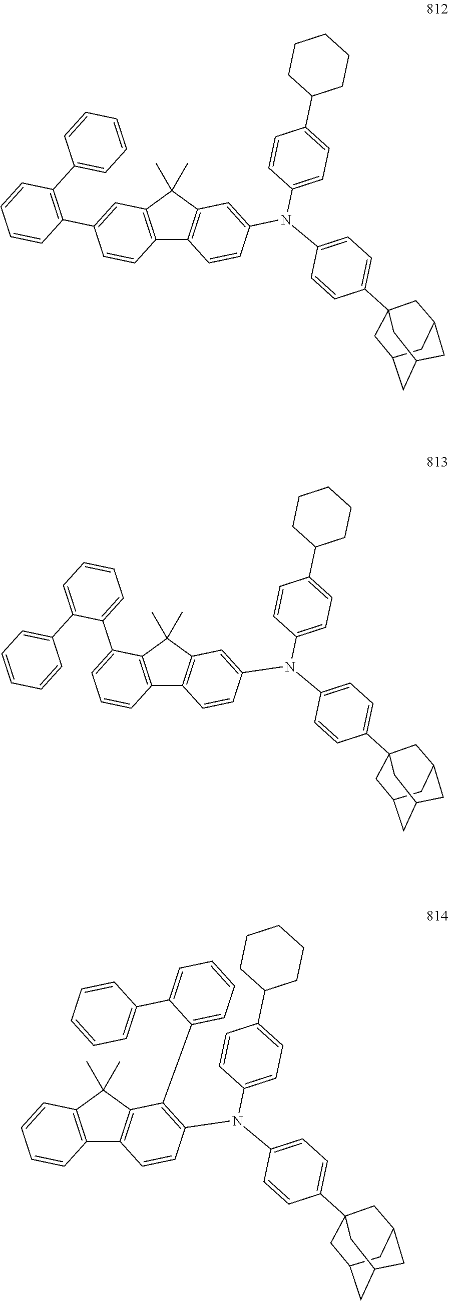 [SMILES: CC1(C)c2cc(-c3ccccc3-c3ccccc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccccc3-c3ccccc3)c21 ; CC1(C)c2ccccc2-c2ccc(N(c3ccc(C4CCCCC4)cc3)c3ccc(C45CC6CC(CC(C6)C4)C5)cc3)c(-c3ccccc3-c3ccccc3)c21] 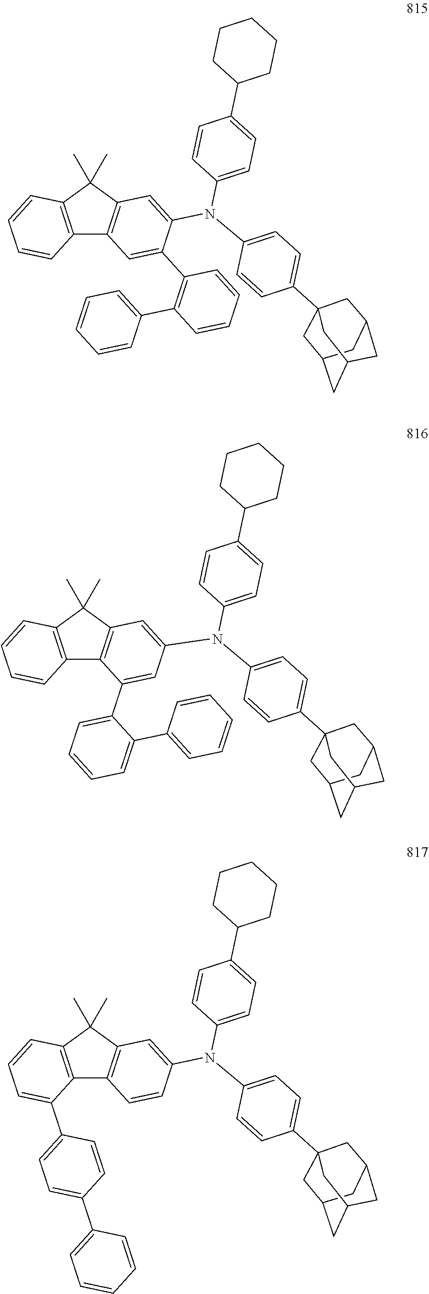 [SMILES: CC1(C)c2ccccc2-c2cc(-c3ccccc3-c3ccccc3)c(N(c3ccc(C4CCCCC4)cc3)c3ccc(C45CC6CC(CC(C6)C4)C5)cc3)cc21 ; CC1(C)c2ccccc2-c2c(-c3ccccc3-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc(-c4ccccc4)cc3)cccc21] 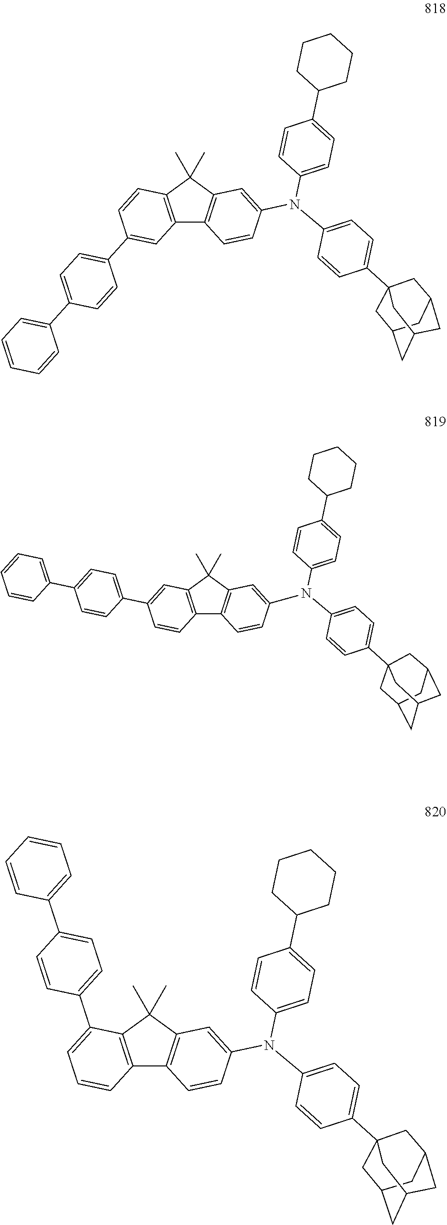 [SMILES: CC1(C)c2ccc(-c3ccc(-c4ccccc4)cc3)cc2-c2ccc(N(c3ccc(C4CCCCC4)cc3)c3ccc(C45CC6CC(CC(C6)C4)C5)cc3)cc21 ; CC1(C)c2cc(-c3ccc(-c4ccccc4)cc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc(-c4ccccc4)cc3)c21] 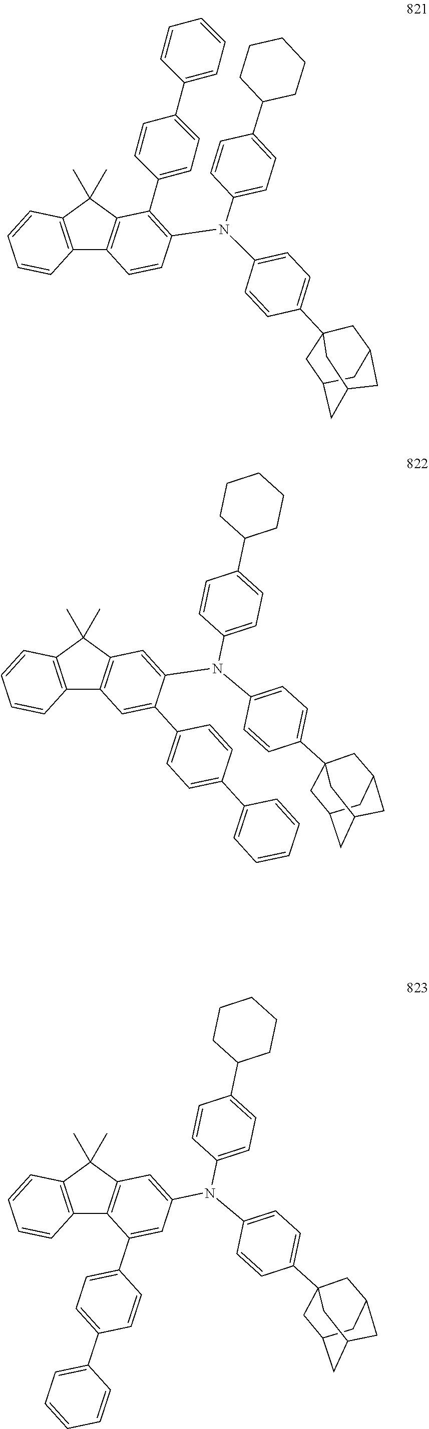 [SMILES: CC1(C)c2ccccc2-c2ccc(N(c3ccc(C4CCCCC4)cc3)c3ccc(C45CC6CC(CC(C6)C4)C5)cc3)c(-c3ccc(-c4ccccc4)cc3)c21 ; CC1(C)c2ccccc2-c2cc(-c3ccc(-c4ccccc4)cc3)c(N(c3ccc(C4CCCCC4)cc3)c3ccc(C45CC6CC(CC(C6)C4)C5)cc3)cc21 ; CC1(C)c2ccccc2-c2c(-c3ccc(-c4ccccc4)cc3)cc(N(c3ccc(C4CCCCC4)cc3)c3ccc(C45CC6CC(CC(C6)C4)C5)cc3)cc21] 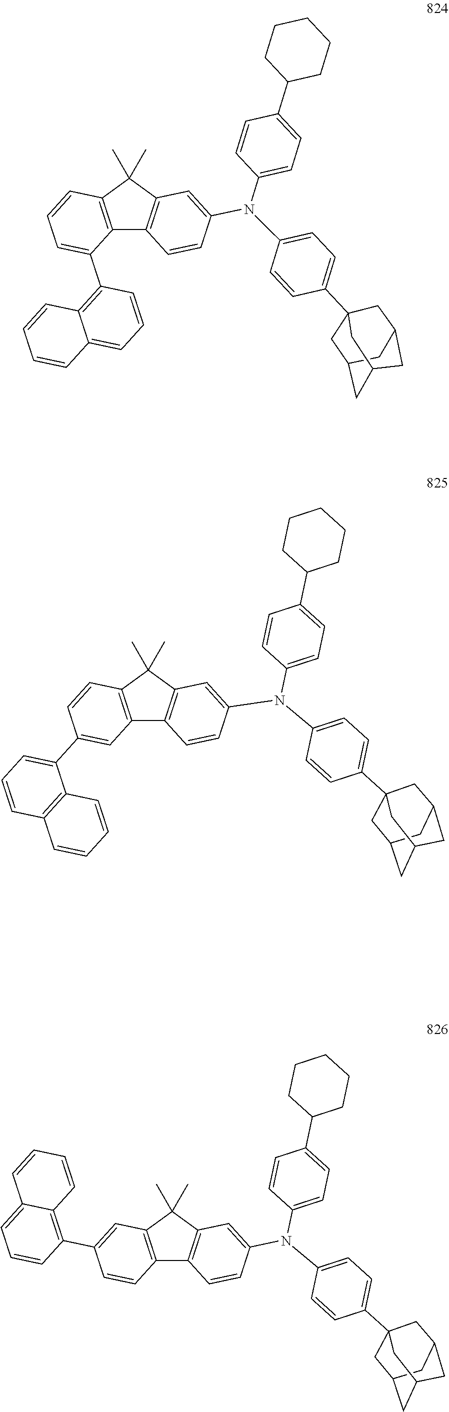 [SMILES: CC1(C)c2cc(N(c3ccc(C4CCCCC4)cc3)c3ccc(C45CC6CC(CC(C6)C4)C5)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C45CC6CC(CC(C6)C4)C5)cc3)cc21 ; CC1(C)c2cc(-c3cccc4ccccc34)ccc2-c2ccc(N(c3ccc(C4CCCCC4)cc3)c3ccc(C45CC6CC(CC(C6)C4)C5)cc3)cc21] 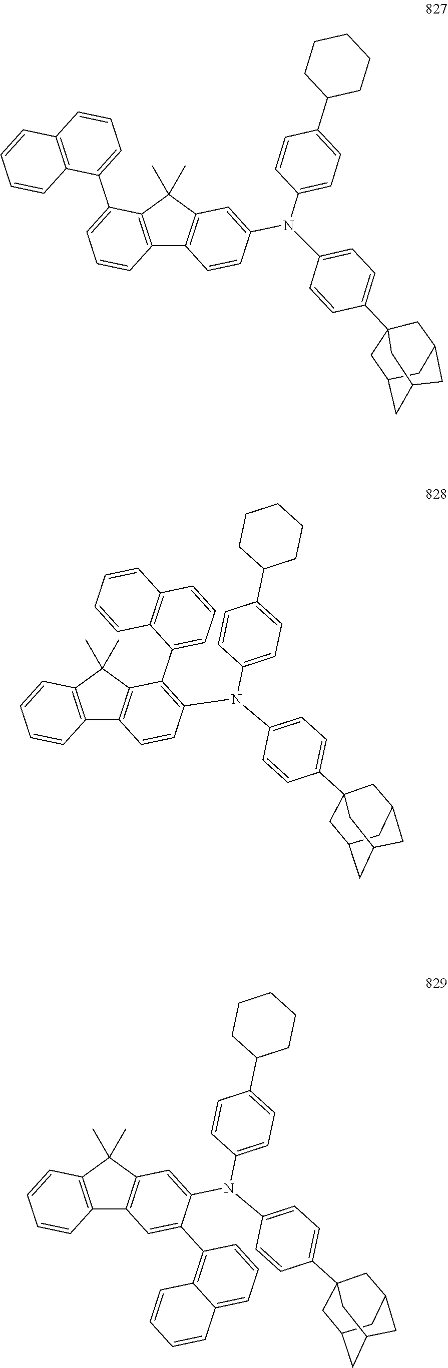 [SMILES: CC1(C)c2cc(N(c3ccc(C4CCCCC4)cc3)c3ccc(C45CC6CC(CC(C6)C4)C5)cc3)ccc2-c2cccc(-c3cccc4ccccc34)c21 ; CC1(C)c2ccccc2-c2ccc(N(c3ccc(C4CCCCC4)cc3)c3ccc(C45CC6CC(CC(C6)C4)C5)cc3)c(-c3cccc4ccccc34)c21 ; CC1(C)c2ccccc2-c2cc(-c3cccc4ccccc34)c(N(c3ccc(C4CCCCC4)cc3)c3ccc(C45CC6CC(CC(C6)C4)C5)cc3)cc21] 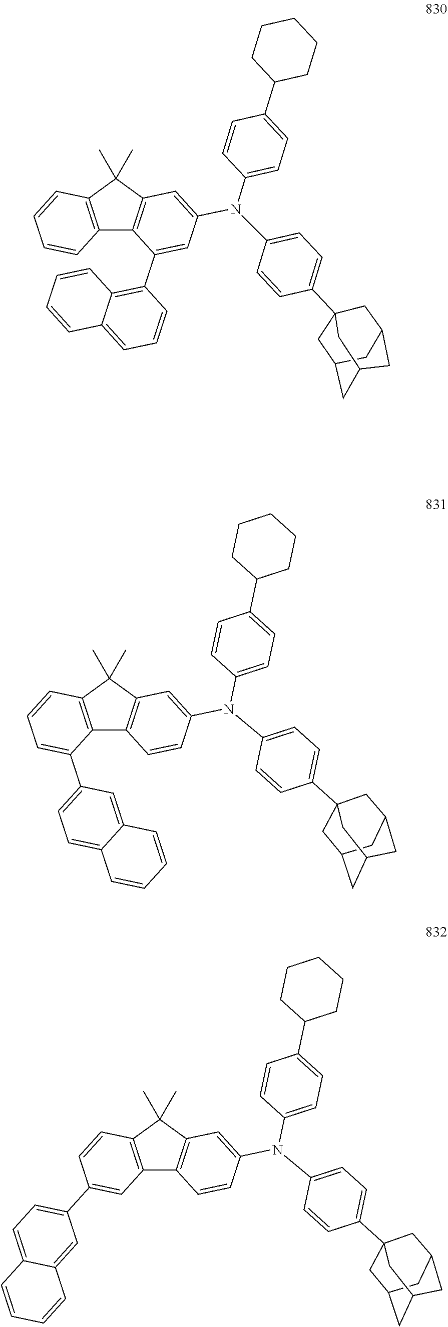 [SMILES: CC1(C)c2ccccc2-c2c(-c3cccc4ccccc34)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C45CC6CC(CC(C6)C4)C5)cc3)cc21] 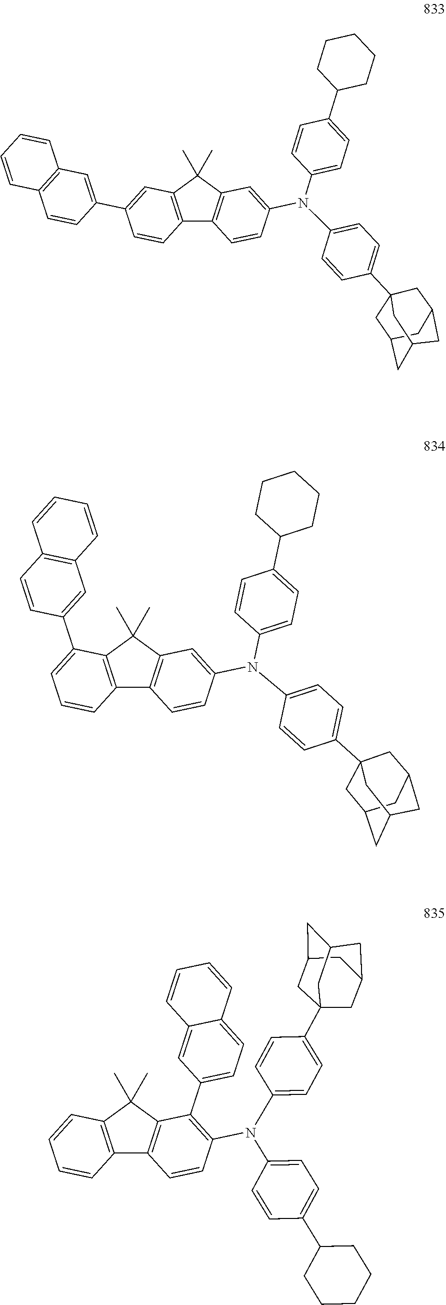 [SMILES: CC1(C)c2cc(-c3ccc4ccccc4c3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc4ccccc4c3)c21 ; CC1(C)c2ccccc2-c2ccc(N(c3ccc(C4CCCCC4)cc3)c3ccc(C45CC6CC(CC(C6)C4)C5)cc3)c(-c3ccc4ccccc4c3)c21] 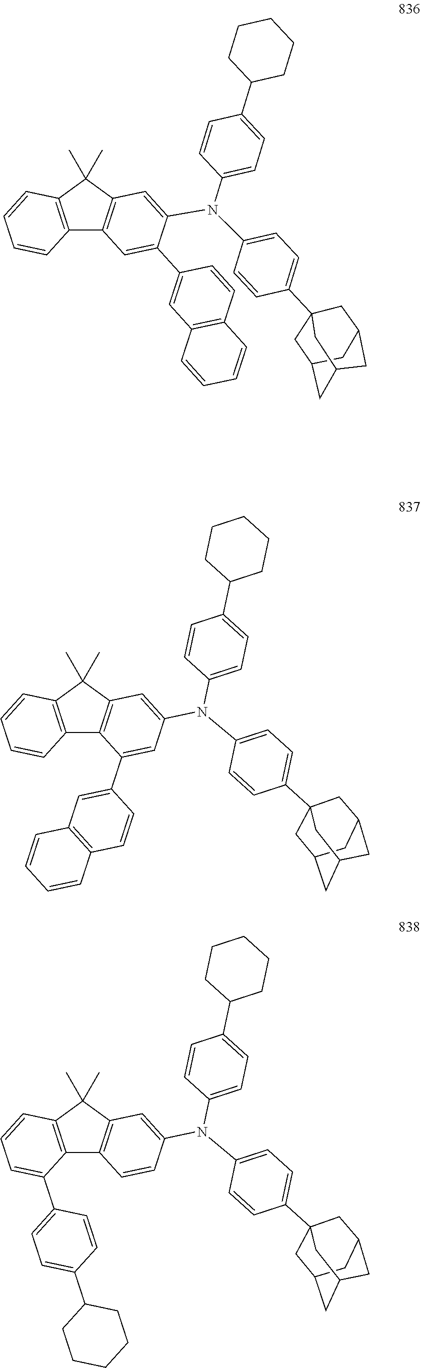 [SMILES: CC1(C)c2ccccc2-c2cc(-c3ccc4ccccc4c3)c(N(c3ccc(C4CCCCC4)cc3)c3ccc(C45CC6CC(CC(C6)C4)C5)cc3)cc21 ; CC1(C)c2ccccc2-c2c(-c3ccc4ccccc4c3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2c(-c3ccc(C4CCCCC4)cc3)cccc21] 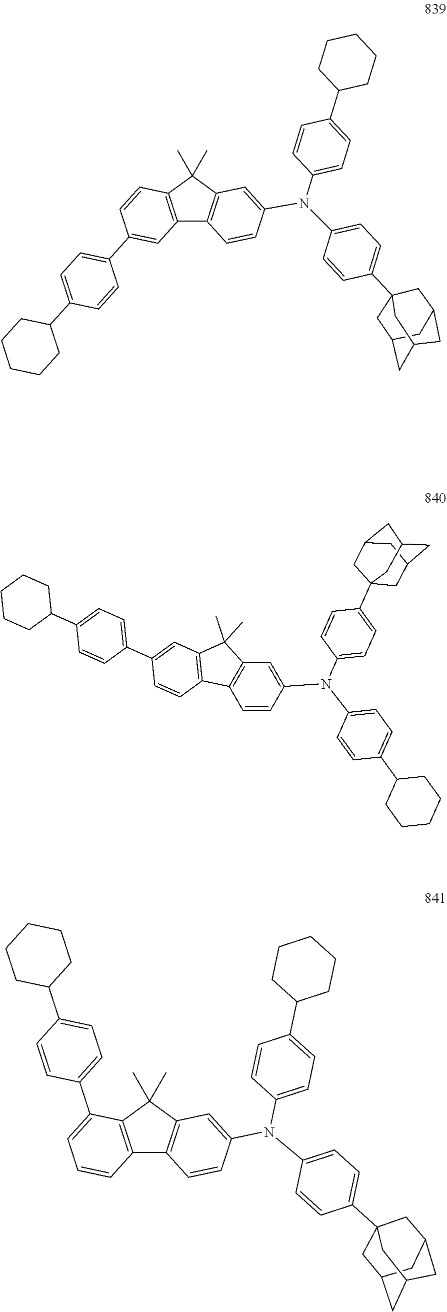 [SMILES: CC1(C)c2ccc(-c3ccc(C4CCCCC4)cc3)cc2-c2ccc(N(c3ccc(C4CCCCC4)cc3)c3ccc(C45CC6CC(CC(C6)C4)C5)cc3)cc21 ; CC1(C)c2cc(-c3ccc(C4CCCCC4)cc3)ccc2-c2ccc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)ccc2-c2cccc(-c3ccc(C4CCCCC4)cc3)c21] 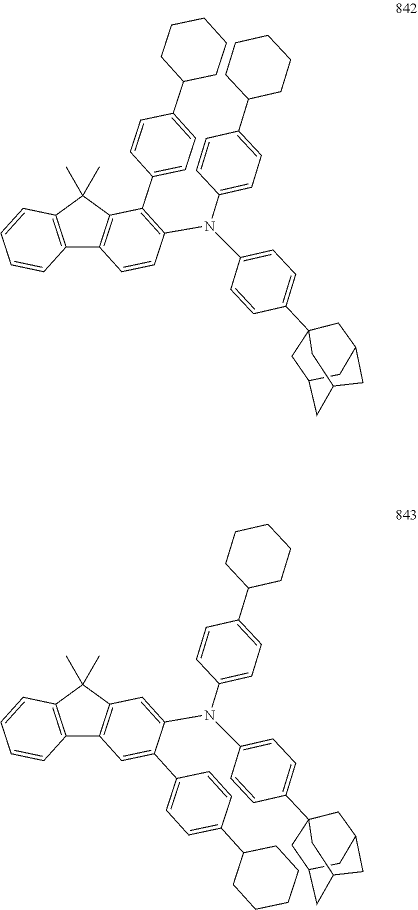 [SMILES: CC1(C)c2ccccc2-c2ccc(N(c3ccc(C4CCCCC4)cc3)c3ccc(C45CC6CC(CC(C6)C4)C5)cc3)c(-c3ccc(C4CCCCC4)cc3)c21 ; CC1(C)c2ccccc2-c2cc(-c3ccc(C4CCCCC4)cc3)c(N(c3ccc(C4CCCCC4)cc3)c3ccc(C45CC6CC(CC(C6)C4)C5)cc3)cc21] 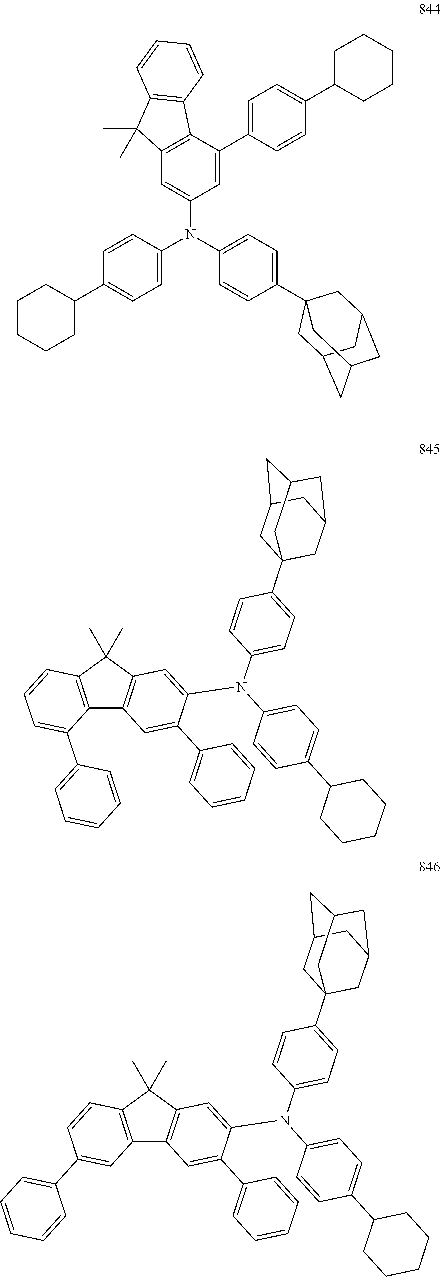 [SMILES: CC1(C)c2ccccc2-c2c(-c3ccc(C4CCCCC4)cc3)cc(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)c(-c3ccccc3)cc2-c2c(-c3ccccc3)cccc21 ; CC1(C)c2ccc(-c3ccccc3)cc2-c2cc(-c3ccccc3)c(N(c3ccc(C4CCCCC4)cc3)c3ccc(C45CC6CC(CC(C6)C4)C5)cc3)cc21] 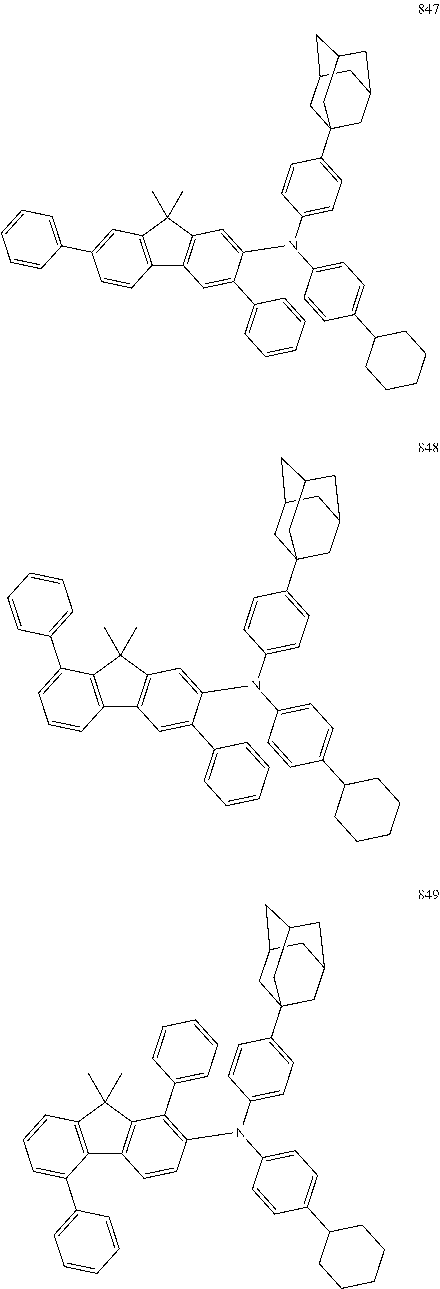 [SMILES: CC1(C)c2cc(-c3ccccc3)ccc2-c2cc(-c3ccccc3)c(N(c3ccc(C4CCCCC4)cc3)c3ccc(C45CC6CC(CC(C6)C4)C5)cc3)cc21 ; CC1(C)c2cc(N(c3ccc(C4CCCCC4)cc3)c3ccc(C45CC6CC(CC(C6)C4)C5)cc3)c(-c3ccccc3)cc2-c2cccc(-c3ccccc3)c21 ; CC1(C)c2cccc(-c3ccccc3)c2-c2ccc(N(c3ccc(C4CCCCC4)cc3)c3ccc(C45CC6CC(CC(C6)C4)C5)cc3)c(-c3ccccc3)c21] 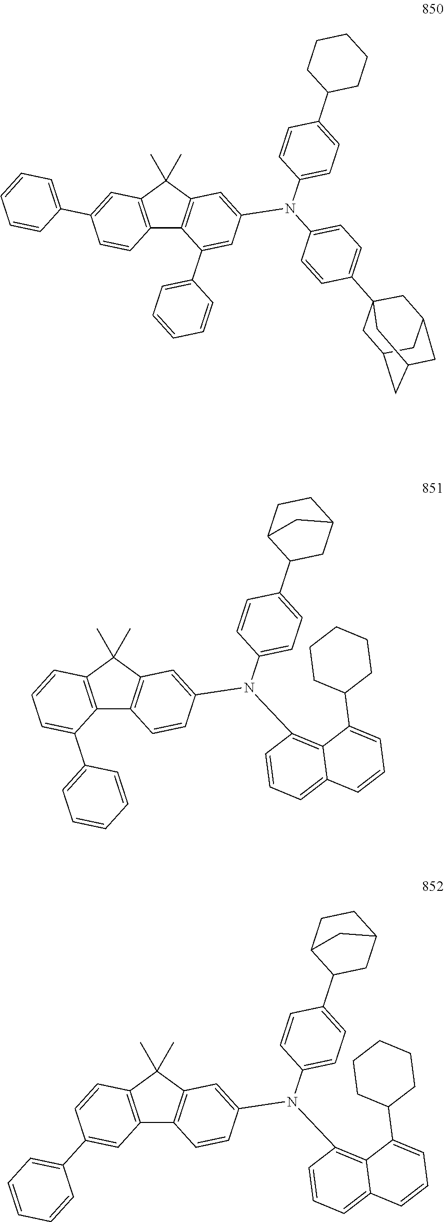 [SMILES: CC1(C)c2cc(-c3ccccc3)ccc2-c2c(-c3ccccc3)cc(N(c3ccc(C4CCCCC4)cc3)c3ccc(C45CC6CC(CC(C6)C4)C5)cc3)cc21 ; CC1(C)c2cc(N(c3ccc(C4CC5CCC4C5)cc3)c3cccc4cccc(C5CCCCC5)c34)ccc2-c2c(-c3ccccc3)cccc21 ; CC1(C)c2ccc(-c3ccccc3)cc2-c2ccc(N(c3ccc(C4CC5CCC4C5)cc3)c3cccc4cccc(C5CCCCC5)c34)cc21] 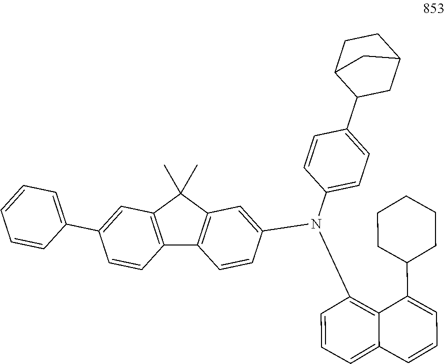 [SMILES: CC1(C)c2cc(-c3ccccc3)ccc2-c2ccc(N(c3ccc(C4CC5CCC4C5)cc3)c3cccc4cccc(C5CCCCC5)c34)cc21] 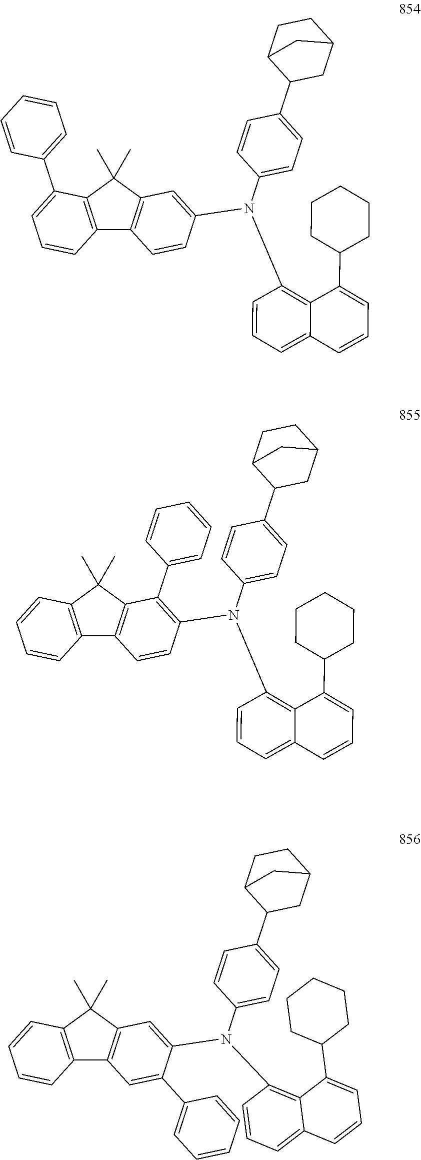 [SMILES: CC1(C)c2cc(N(c3ccc(C4CC5CCC4C5)cc3)c3cccc4cccc(C5CCCCC5)c34)ccc2-c2cccc(-c3ccccc3)c21 ; CC1(C)c2ccccc2-c2ccc(N(c3ccc(C4CC5CCC4C5)cc3)c3cccc4cccc(C5CCCCC5)c34)c(-c3ccccc3)c21 ; CC1(C)c2ccccc2-c2cc(-c3ccccc3)c(N(c3ccc(C4CC5CCC4C5)cc3)c3cccc4cccc(C5CCCCC5)c34)cc21] 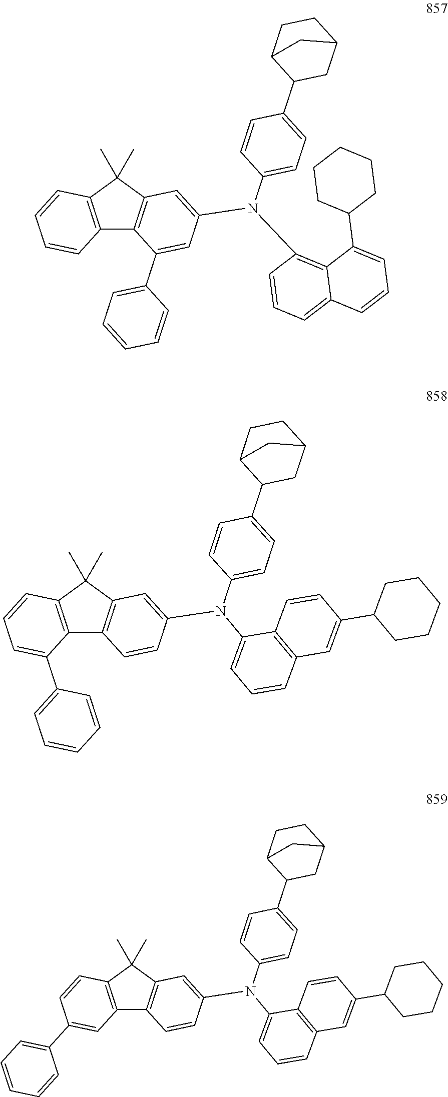 [SMILES: CC1(C)c2ccccc2-c2c(-c3ccccc3)cc(N(c3ccc(C4CC5CCC4C5)cc3)c3cccc4cccc(C5CCCCC5)c34)cc21 ; CC1(C)c2cc(N(c3ccc(C4CC5CCC4C5)cc3)c3cccc4cc(C5CCCCC5)ccc34)ccc2-c2c(-c3ccccc3)cccc21 ; CC1(C)c2ccc(-c3ccccc3)cc2-c2ccc(N(c3ccc(C4CC5CCC4C5)cc3)c3cccc4cc(C5CCCCC5)ccc34)cc21] 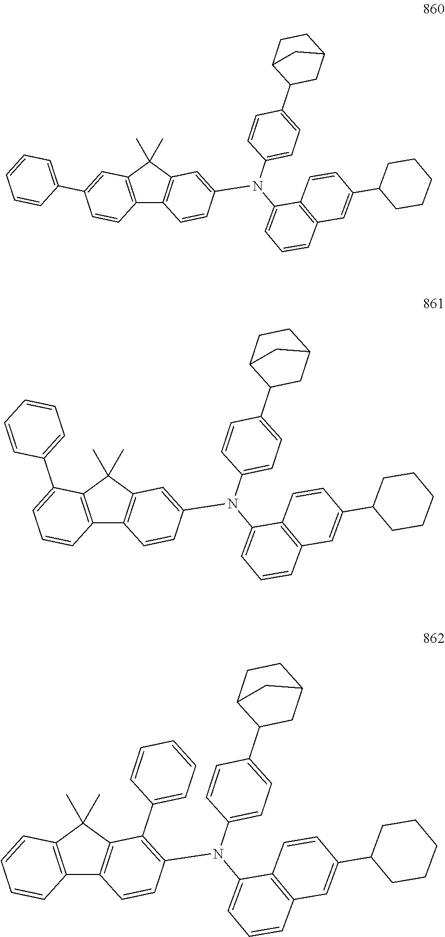 [SMILES: CC1(C)c2cc(-c3ccccc3)ccc2-c2ccc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3cccc4cc(C5CCCCC5)ccc34)ccc2-c2cccc(-c3ccccc3)c21 ; CC1(C)c2ccccc2-c2ccc(N(c3ccc(C4CC5CCC4C5)cc3)c3cccc4cc(C5CCCCC5)ccc34)c(-c3ccccc3)c21] 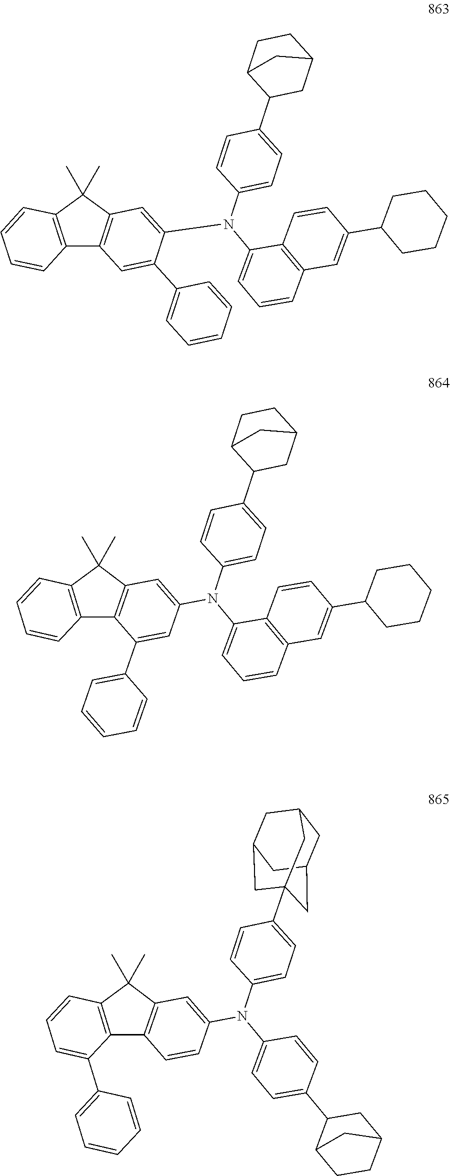 [SMILES: CC1(C)c2ccccc2-c2cc(-c3ccccc3)c(N(c3ccc(C4CC5CCC4C5)cc3)c3cccc4cc(C5CCCCC5)ccc34)cc21 ; CC1(C)c2ccccc2-c2c(-c3ccccc3)cc(N(c3ccc(C4CC5CCC4C5)cc3)c3cccc4cc(C5CCCCC5)ccc34)cc21 ; CC1(C)c2cc(N(c3ccc(C4CC5CCC4C5)cc3)c3ccc(C45CC6CC(CC(C6)C4)C5)cc3)ccc2-c2c(-c3ccccc3)cccc21] 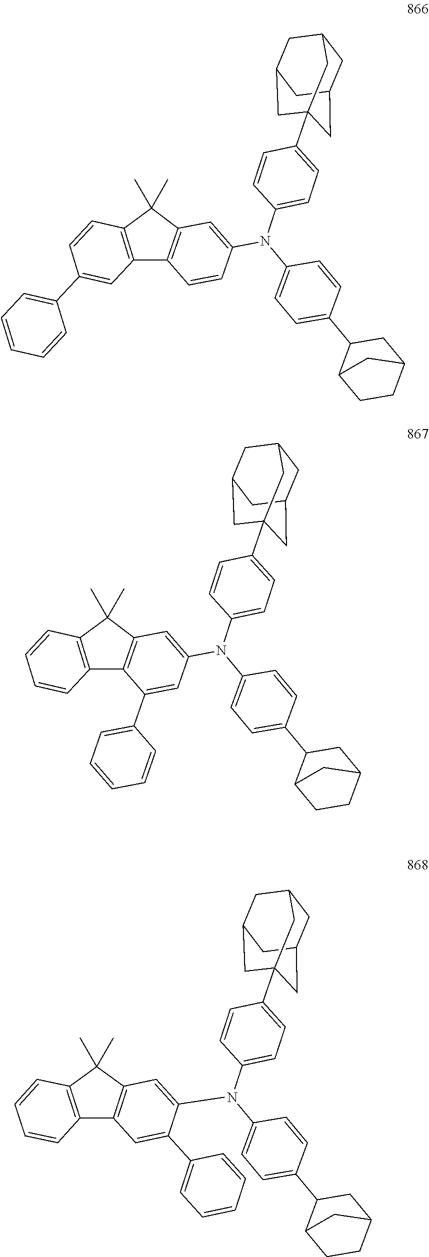 [SMILES: CC1(C)c2ccc(-c3ccccc3)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3ccccc3)cc(N(c3ccc(C4CC5CCC4C5)cc3)c3ccc(C45CC6CC(CC(C6)C4)C5)cc3)cc21 ; CC1(C)c2ccccc2-c2cc(-c3ccccc3)c(N(c3ccc(C4CC5CCC4C5)cc3)c3ccc(C45CC6CC(CC(C6)C4)C5)cc3)cc21] 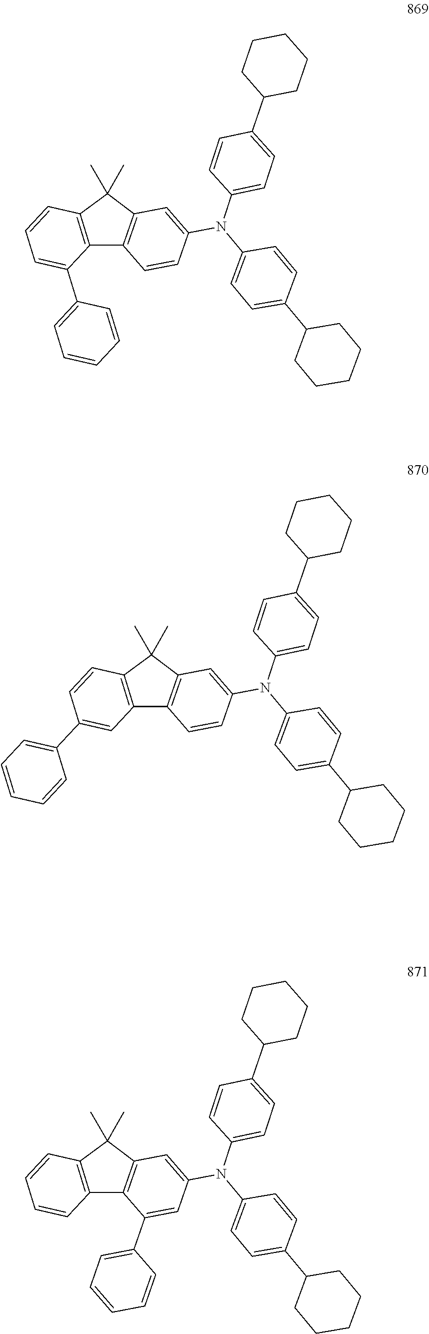 [SMILES: CC1(C)c2cc(N(c3ccc(C4CCCCC4)cc3)c3ccc(C4CCCCC4)cc3)ccc2-c2c(-c3ccccc3)cccc21 ; CC1(C)c2ccc(-c3ccccc3)cc2-c2ccc(N(c3ccc(C4CCCCC4)cc3)c3ccc(C4CCCCC4)cc3)cc21 ; CC1(C)c2ccccc2-c2c(-c3ccccc3)cc(N(c3ccc(C4CCCCC4)cc3)c3ccc(C4CCCCC4)cc3)cc21] 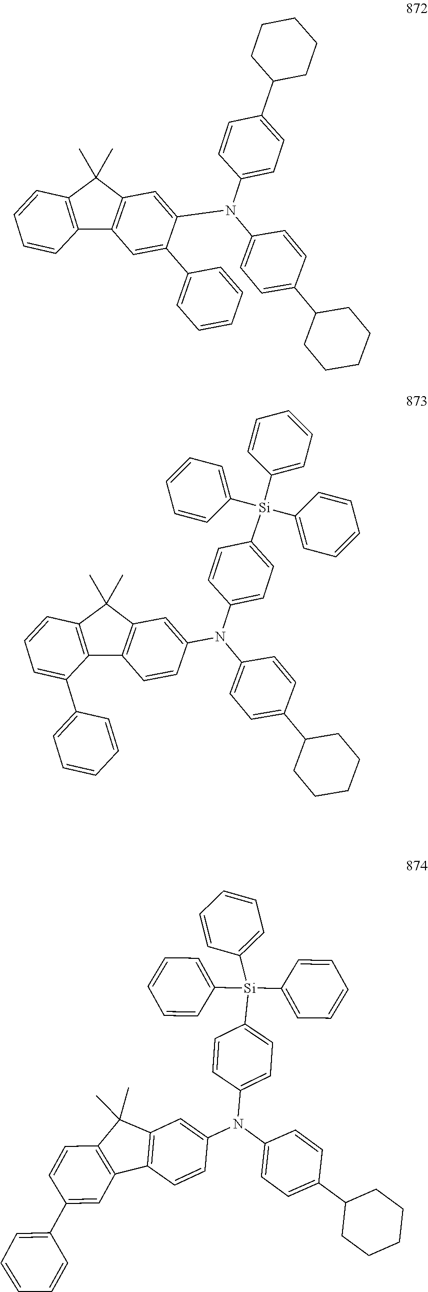 [SMILES: CC1(C)c2ccccc2-c2cc(-c3ccccc3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccccc3)cccc21 ; CC1(C)c2ccc(-c3ccccc3)cc2-c2ccc(N(c3ccc(C4CCCCC4)cc3)c3ccc([Si](c4ccccc4)(c4ccccc4)c4ccccc4)cc3)cc21] 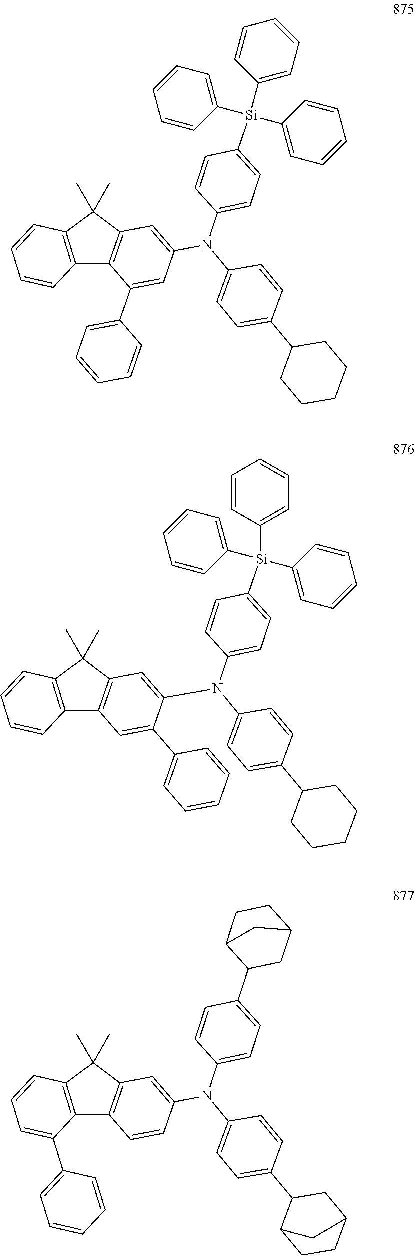 [SMILES: CC1(C)c2ccccc2-c2c(-c3ccccc3)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3ccccc3)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3ccccc3)cccc21] 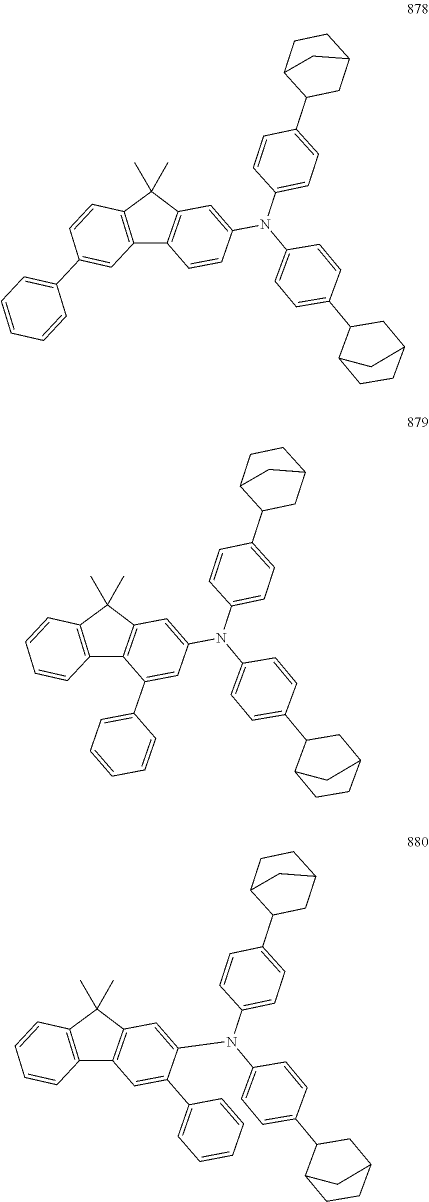 [SMILES: CC1(C)c2ccc(-c3ccccc3)cc2-c2ccc(N(c3ccc(C4CC5CCC4C5)cc3)c3ccc(C4CC5CCC4C5)cc3)cc21 ; CC1(C)c2ccccc2-c2c(-c3ccccc3)cc(N(c3ccc(C4CC5CCC4C5)cc3)c3ccc(C4CC5CCC4C5)cc3)cc21 ; CC1(C)c2ccccc2-c2cc(-c3ccccc3)c(N(c3ccc(C4CC5CCC4C5)cc3)c3ccc(C4CC5CCC4C5)cc3)cc21] 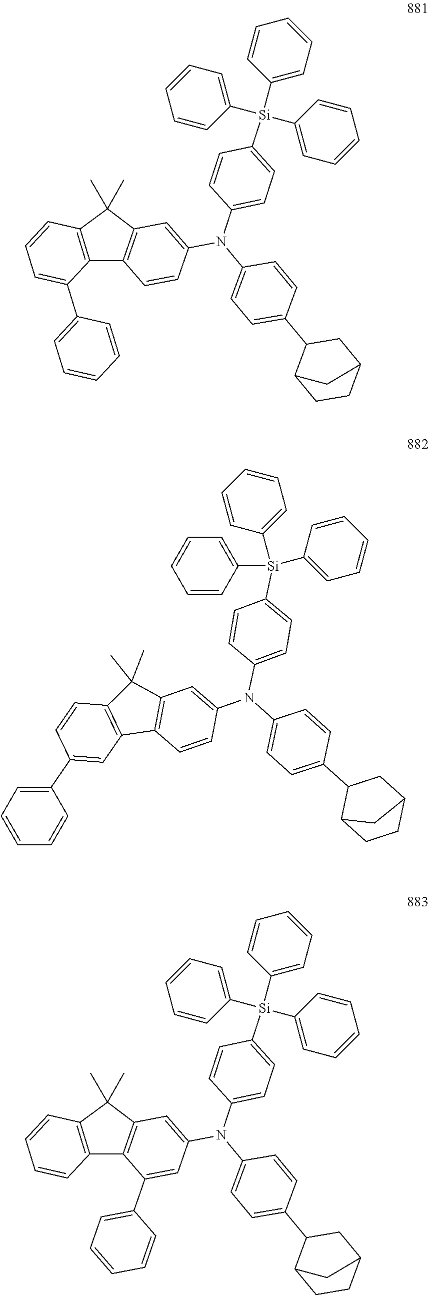 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3ccccc3)cccc21 ; CC1(C)c2ccc(-c3ccccc3)cc2-c2ccc(N(c3ccc(C4CC5CCC4C5)cc3)c3ccc([Si](c4ccccc4)(c4ccccc4)c4ccccc4)cc3)cc21 ; CC1(C)c2ccccc2-c2c(-c3ccccc3)cc(N(c3ccc(C4CC5CCC4C5)cc3)c3ccc([Si](c4ccccc4)(c4ccccc4)c4ccccc4)cc3)cc21] 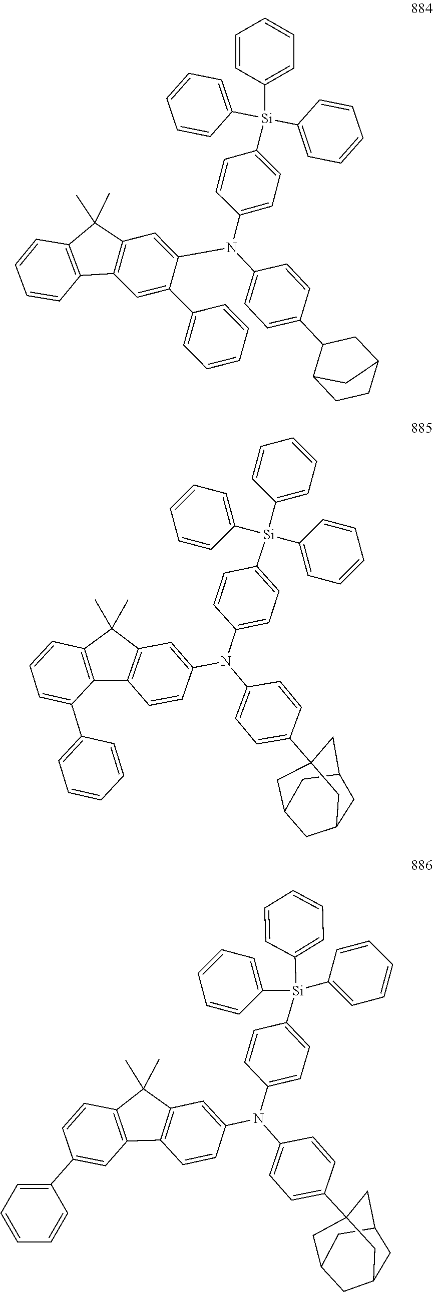 [SMILES: CC1(C)c2ccccc2-c2cc(-c3ccccc3)c(N(c3ccc(C4CC5CCC4C5)cc3)c3ccc([Si](c4ccccc4)(c4ccccc4)c4ccccc4)cc3)cc21 ; CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3ccccc3)cccc21 ; CC1(C)c2ccc(-c3ccccc3)cc2-c2ccc(N(c3ccc(C45CC6CC(CC(C6)C4)C5)cc3)c3ccc([Si](c4ccccc4)(c4ccccc4)c4ccccc4)cc3)cc21] 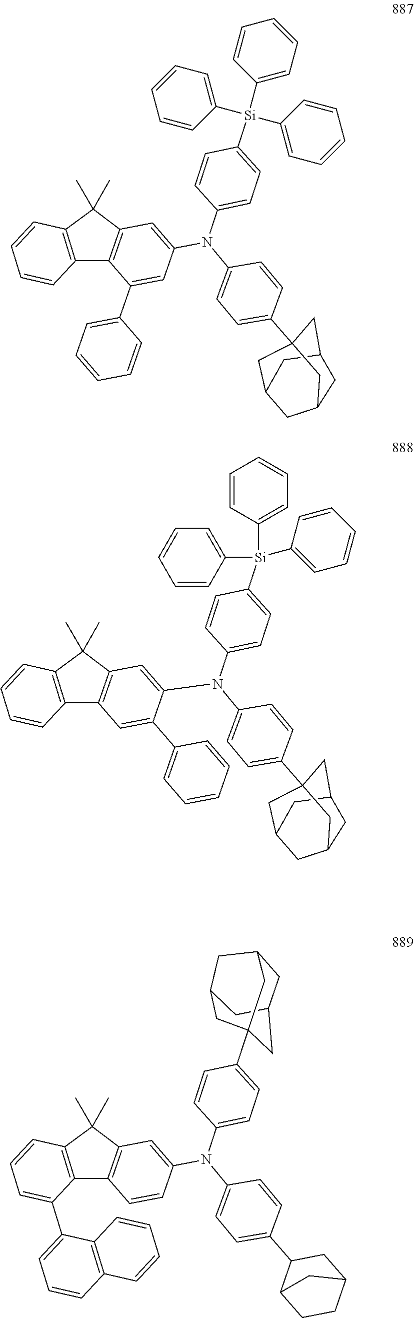 [SMILES: CC1(C)c2ccccc2-c2c(-c3ccccc3)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3ccccc3)c(N(c3ccc(C45CC6CC(CC(C6)C4)C5)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C45CC6CC(CC(C6)C4)C5)cc3)ccc2-c2c(-c3cccc4ccccc34)cccc21] 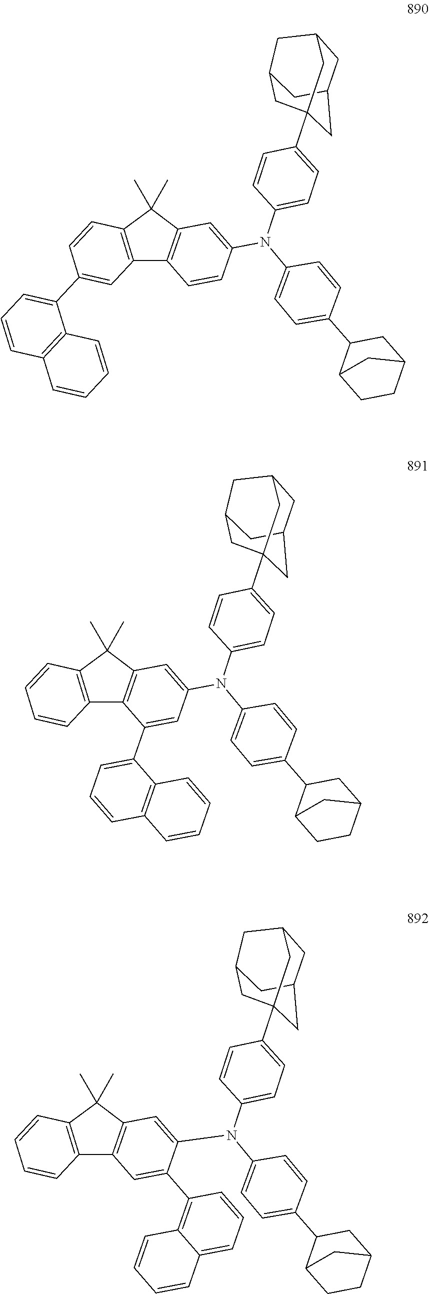 [SMILES: CC1(C)c2ccc(-c3cccc4ccccc34)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3cccc4ccccc34)cc(N(c3ccc(C4CC5CCC4C5)cc3)c3ccc(C45CC6CC(CC(C6)C4)C5)cc3)cc21 ; CC1(C)c2ccccc2-c2cc(-c3cccc4ccccc34)c(N(c3ccc(C4CC5CCC4C5)cc3)c3ccc(C45CC6CC(CC(C6)C4)C5)cc3)cc21] 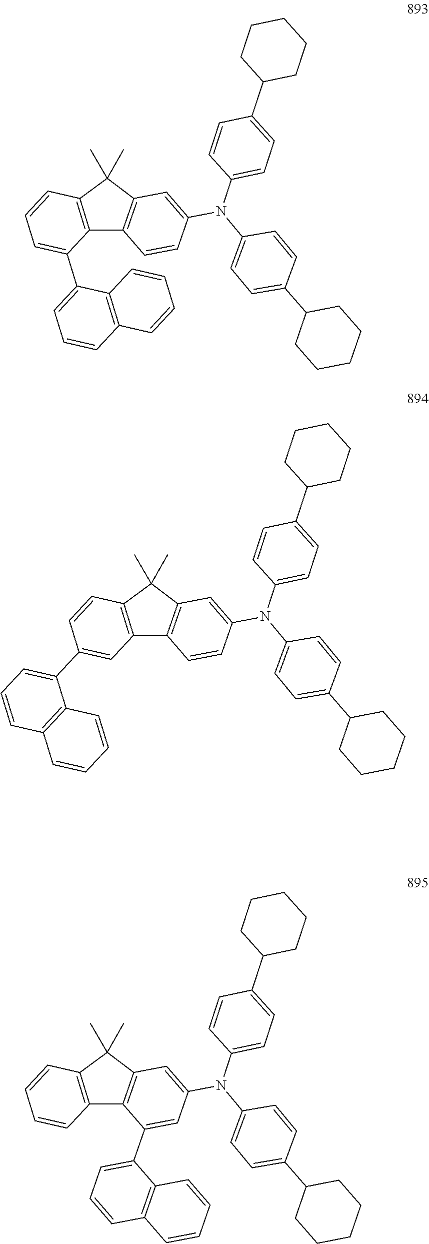 [SMILES: CC1(C)c2cc(N(c3ccc(C4CCCCC4)cc3)c3ccc(C4CCCCC4)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc(C4CCCCC4)cc3)cc21 ; CC1(C)c2ccccc2-c2c(-c3cccc4ccccc34)cc(N(c3ccc(C4CCCCC4)cc3)c3ccc(C4CCCCC4)cc3)cc21] 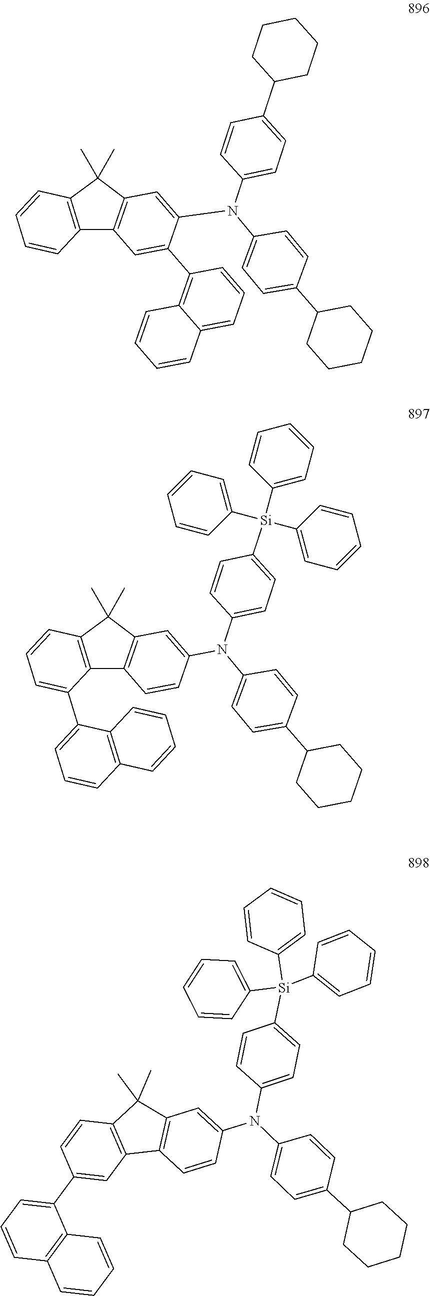 [SMILES: CC1(C)c2ccccc2-c2cc(-c3cccc4ccccc34)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3cccc4ccccc34)cccc21 ; CC1(C)c2ccc(-c3cccc4ccccc34)cc2-c2ccc(N(c3ccc(C4CCCCC4)cc3)c3ccc([Si](c4ccccc4)(c4ccccc4)c4ccccc4)cc3)cc21] 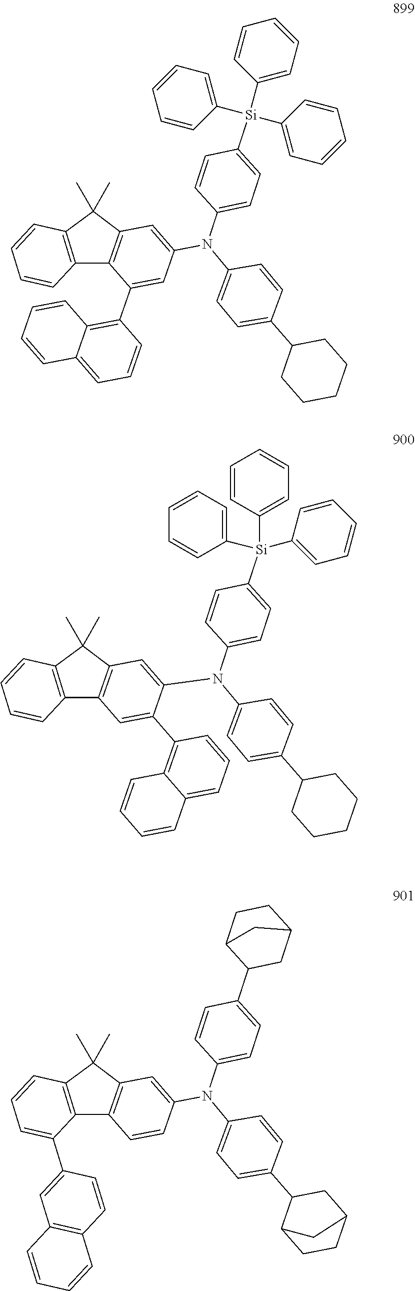 [SMILES: CC1(C)c2ccccc2-c2c(-c3cccc4ccccc34)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3cccc4ccccc34)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3ccc4ccccc4c3)cccc21] 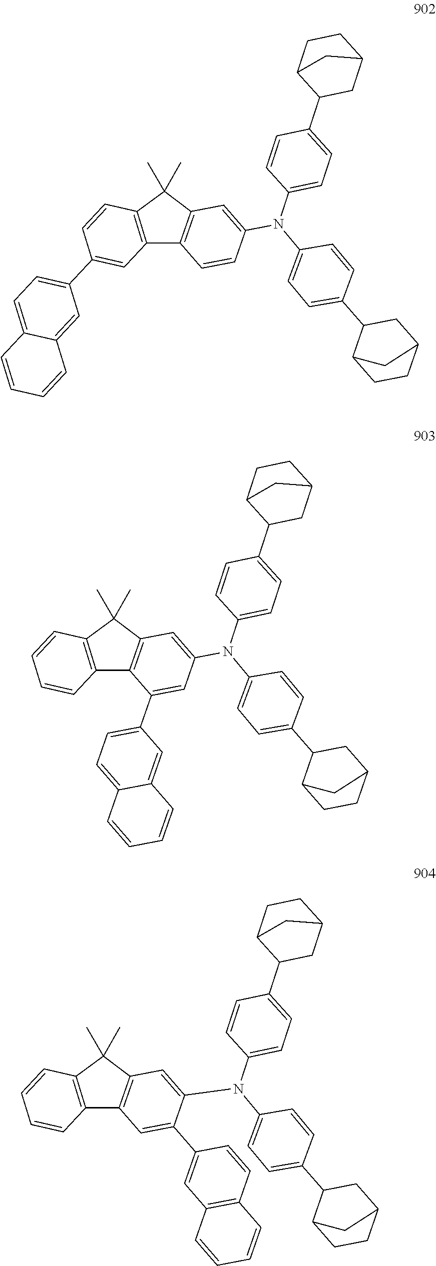 [SMILES: CC1(C)c2ccc(-c3ccc4ccccc4c3)cc2-c2ccc(N(c3ccc(C4CC5CCC4C5)cc3)c3ccc(C4CC5CCC4C5)cc3)cc21 ; CC1(C)c2ccccc2-c2c(-c3ccc4ccccc4c3)cc(N(c3ccc(C4CC5CCC4C5)cc3)c3ccc(C4CC5CCC4C5)cc3)cc21 ; CC1(C)c2ccccc2-c2cc(-c3ccc4ccccc4c3)c(N(c3ccc(C4CC5CCC4C5)cc3)c3ccc(C4CC5CCC4C5)cc3)cc21] 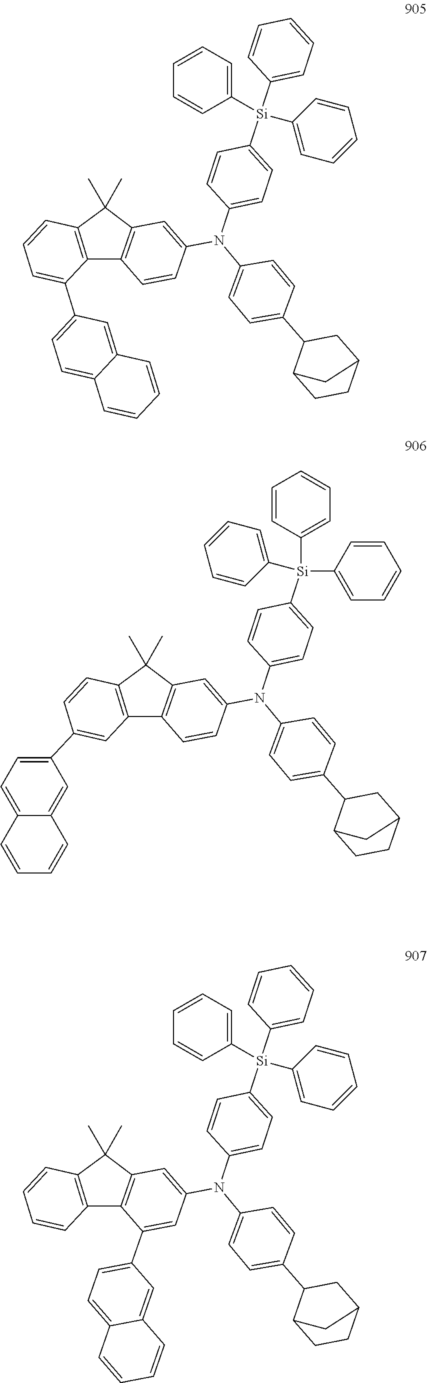 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C4CC5CCC4C5)cc3)c3ccc([Si](c4ccccc4)(c4ccccc4)c4ccccc4)cc3)cc21 ; CC1(C)c2ccccc2-c2c(-c3ccc4ccccc4c3)cc(N(c3ccc(C4CC5CCC4C5)cc3)c3ccc([Si](c4ccccc4)(c4ccccc4)c4ccccc4)cc3)cc21] 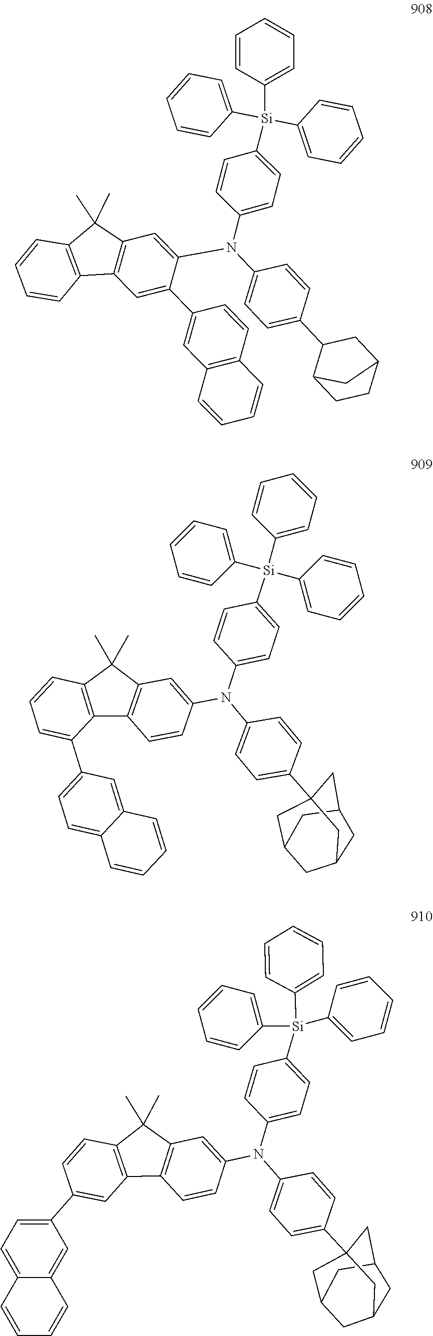 [SMILES: CC1(C)c2ccccc2-c2cc(-c3ccc4ccccc4c3)c(N(c3ccc(C4CC5CCC4C5)cc3)c3ccc([Si](c4ccccc4)(c4ccccc4)c4ccccc4)cc3)cc21 ; CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C45CC6CC(CC(C6)C4)C5)cc3)c3ccc([Si](c4ccccc4)(c4ccccc4)c4ccccc4)cc3)cc21] 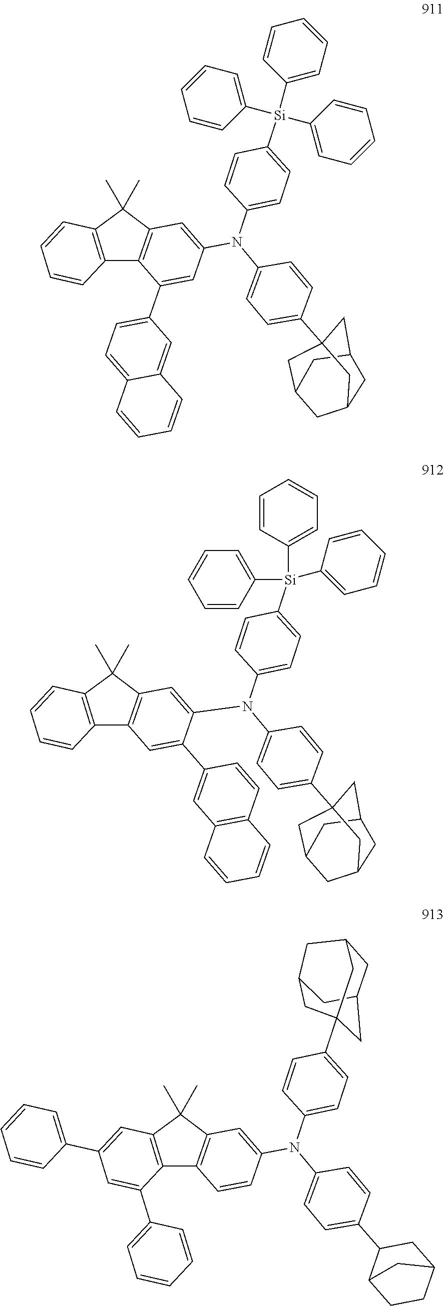 [SMILES: CC1(C)c2ccccc2-c2c(-c3ccc4ccccc4c3)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3ccc4ccccc4c3)c(N(c3ccc(C45CC6CC(CC(C6)C4)C5)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C45CC6CC(CC(C6)C4)C5)cc3)ccc2-c2c(-c3ccccc3)cc(-c3ccccc3)cc21] 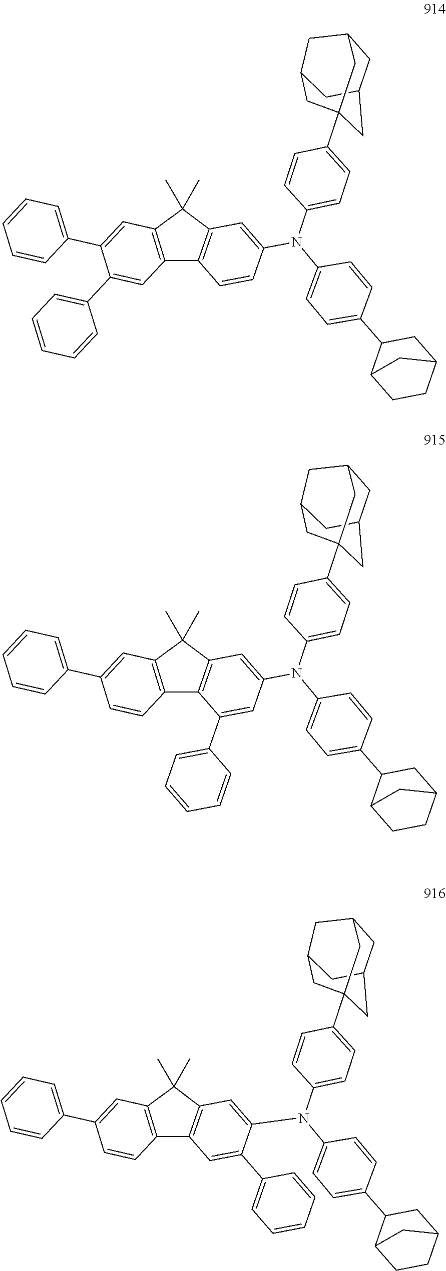 [SMILES: CC1(C)c2cc(N(c3ccc(C4CC5CCC4C5)cc3)c3ccc(C45CC6CC(CC(C6)C4)C5)cc3)ccc2-c2cc(-c3ccccc3)c(-c3ccccc3)cc21 ; CC1(C)c2cc(-c3ccccc3)ccc2-c2c(-c3ccccc3)cc(N(c3ccc(C4CC5CCC4C5)cc3)c3ccc(C45CC6CC(CC(C6)C4)C5)cc3)cc21 ; CC1(C)c2cc(-c3ccccc3)ccc2-c2cc(-c3ccccc3)c(N(c3ccc(C4CC5CCC4C5)cc3)c3ccc(C45CC6CC(CC(C6)C4)C5)cc3)cc21] 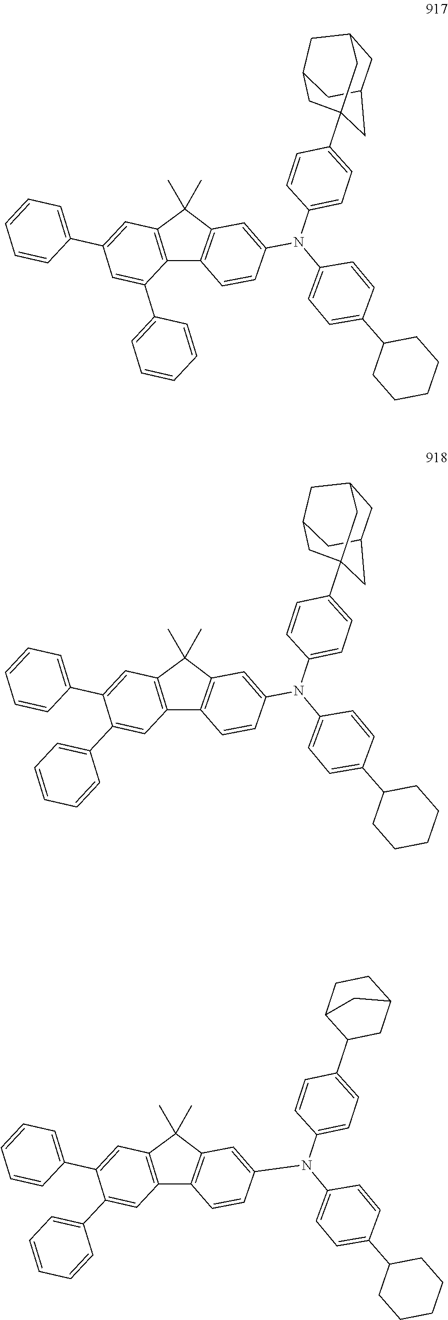 [SMILES: CC1(C)c2cc(N(c3ccc(C4CCCCC4)cc3)c3ccc(C45CC6CC(CC(C6)C4)C5)cc3)ccc2-c2c(-c3ccccc3)cc(-c3ccccc3)cc21 ; CC1(C)c2cc(N(c3ccc(C4CCCCC4)cc3)c3ccc(C45CC6CC(CC(C6)C4)C5)cc3)ccc2-c2cc(-c3ccccc3)c(-c3ccccc3)cc21 ; CC1(C)c2cc(N(c3ccc(C4CCCCC4)cc3)c3ccc(C4CC5CCC4C5)cc3)ccc2-c2cc(-c3ccccc3)c(-c3ccccc3)cc21] 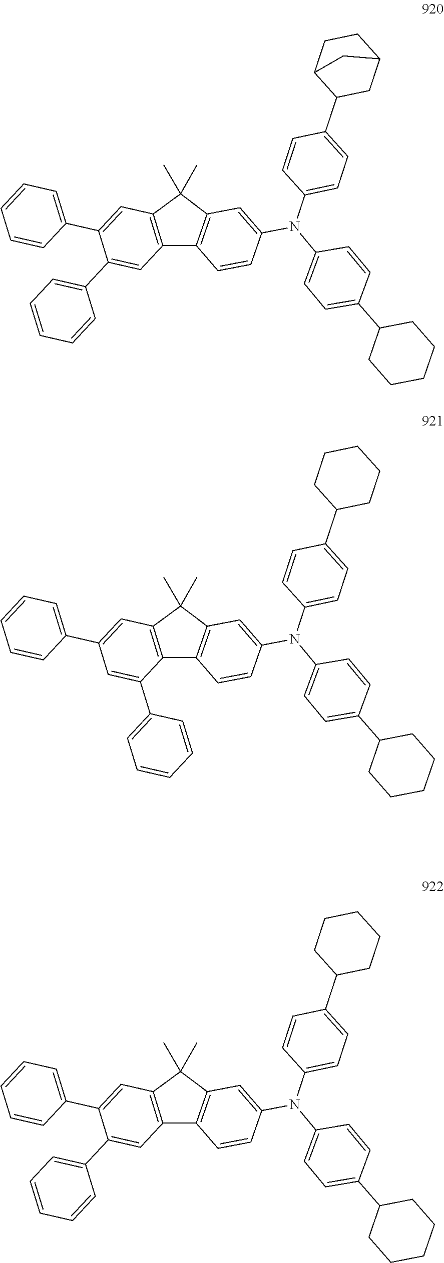 [SMILES: CC1(C)c2cc(N(c3ccc(C4CCCCC4)cc3)c3ccc(C4CC5CCC4C5)cc3)ccc2-c2cc(-c3ccccc3)c(-c3ccccc3)cc21 ; CC1(C)c2cc(N(c3ccc(C4CCCCC4)cc3)c3ccc(C4CCCCC4)cc3)ccc2-c2c(-c3ccccc3)cc(-c3ccccc3)cc21 ; CC1(C)c2cc(N(c3ccc(C4CCCCC4)cc3)c3ccc(C4CCCCC4)cc3)ccc2-c2cc(-c3ccccc3)c(-c3ccccc3)cc21] 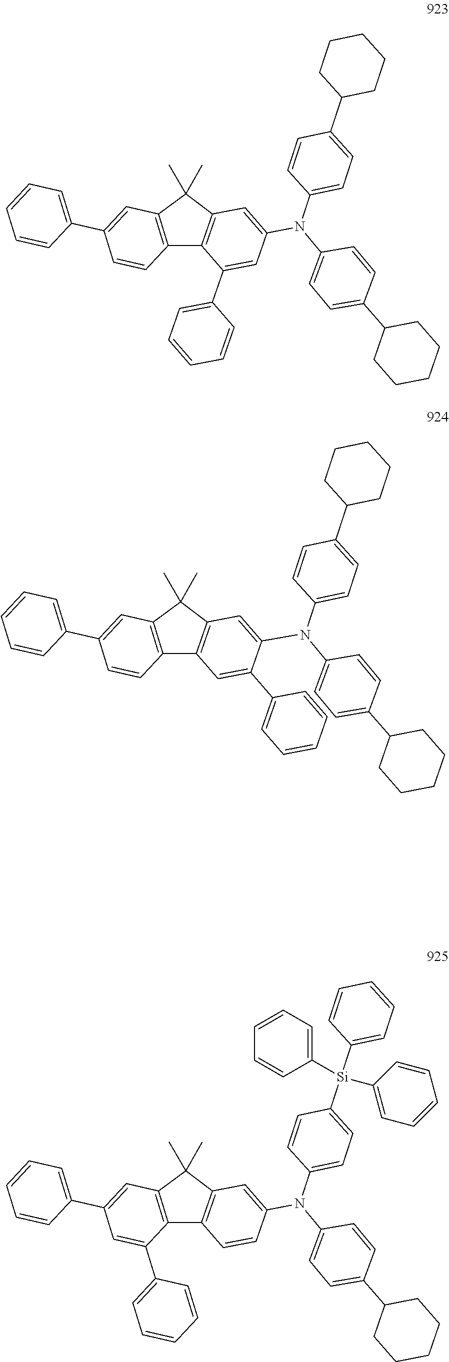 [SMILES: CC1(C)c2cc(-c3ccccc3)ccc2-c2c(-c3ccccc3)cc(N(c3ccc(C4CCCCC4)cc3)c3ccc(C4CCCCC4)cc3)cc21 ; CC1(C)c2cc(-c3ccccc3)ccc2-c2cc(-c3ccccc3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccccc3)cc(-c3ccccc3)cc21] 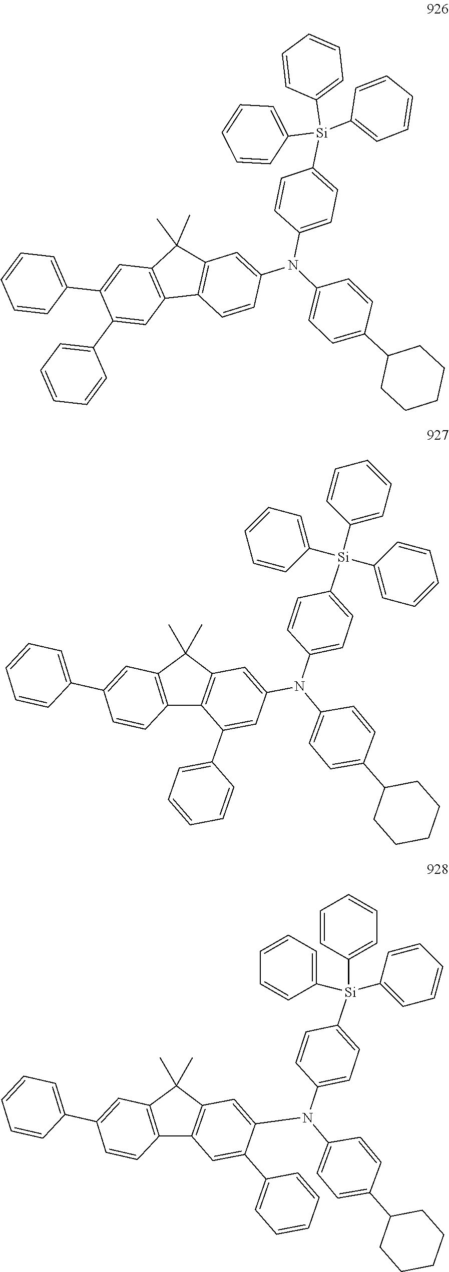 [SMILES: CC1(C)c2cc(N(c3ccc(C4CCCCC4)cc3)c3ccc([Si](c4ccccc4)(c4ccccc4)c4ccccc4)cc3)ccc2-c2cc(-c3ccccc3)c(-c3ccccc3)cc21 ; CC1(C)c2cc(-c3ccccc3)ccc2-c2c(-c3ccccc3)cc(N(c3ccc(C4CCCCC4)cc3)c3ccc([Si](c4ccccc4)(c4ccccc4)c4ccccc4)cc3)cc21 ; CC1(C)c2cc(-c3ccccc3)ccc2-c2cc(-c3ccccc3)c(N(c3ccc(C4CCCCC4)cc3)c3ccc([Si](c4ccccc4)(c4ccccc4)c4ccccc4)cc3)cc21] 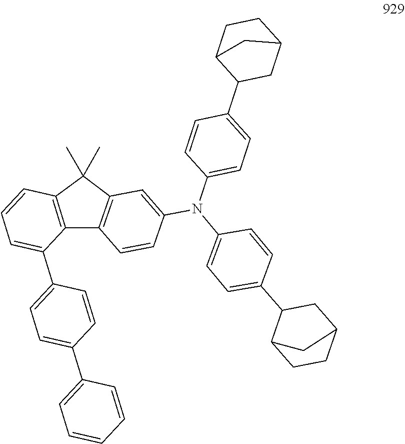 [SMILES: CC1(C)c2cc(N(c3ccc(C4CC5CCC4C5)cc3)c3ccc(C4CC5CCC4C5)cc3)ccc2-c2c(-c3ccc(-c4ccccc4)cc3)cccc21] 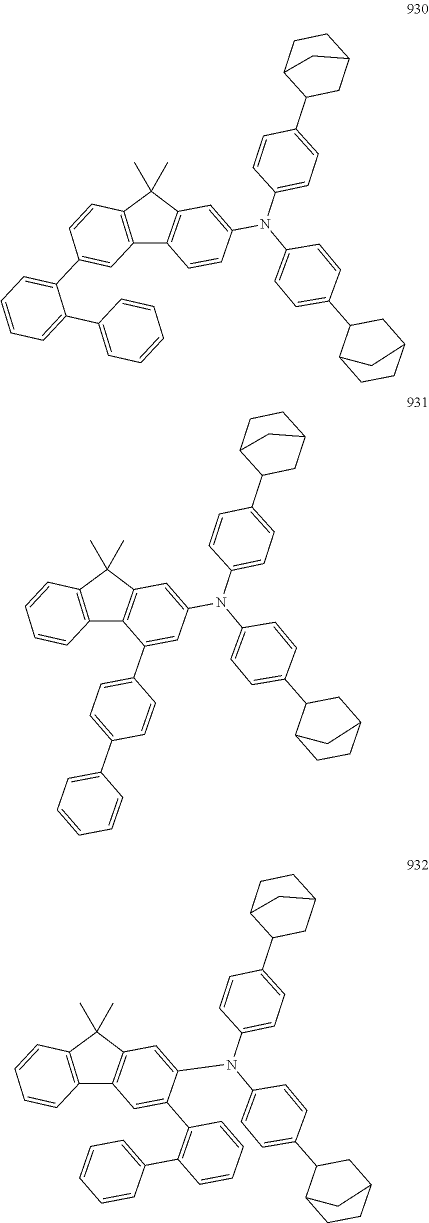 [SMILES: CC1(C)c2ccc(-c3ccccc3-c3ccccc3)cc2-c2ccc(N(c3ccc(C4CC5CCC4C5)cc3)c3ccc(C4CC5CCC4C5)cc3)cc21 ; CC1(C)c2ccccc2-c2c(-c3ccc(-c4ccccc4)cc3)cc(N(c3ccc(C4CC5CCC4C5)cc3)c3ccc(C4CC5CCC4C5)cc3)cc21 ; CC1(C)c2ccccc2-c2cc(-c3ccccc3-c3ccccc3)c(N(c3ccc(C4CC5CCC4C5)cc3)c3ccc(C4CC5CCC4C5)cc3)cc21] 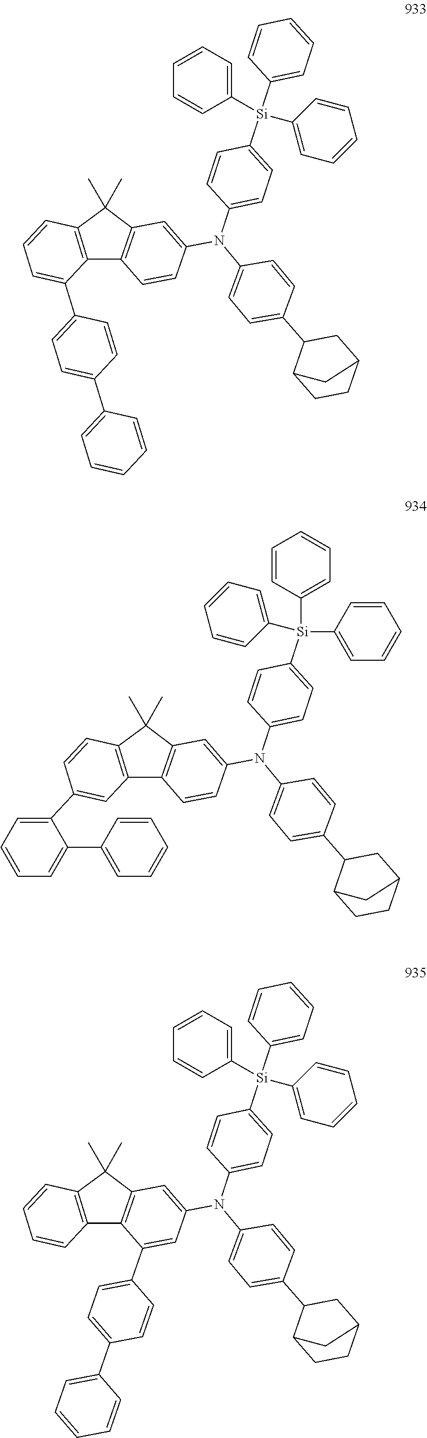 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3ccc(-c4ccccc4)cc3)cccc21 ; CC1(C)c2ccc(-c3ccccc3-c3ccccc3)cc2-c2ccc(N(c3ccc(C4CC5CCC4C5)cc3)c3ccc([Si](c4ccccc4)(c4ccccc4)c4ccccc4)cc3)cc21 ; CC1(C)c2ccccc2-c2c(-c3ccc(-c4ccccc4)cc3)cc(N(c3ccc(C4CC5CCC4C5)cc3)c3ccc([Si](c4ccccc4)(c4ccccc4)c4ccccc4)cc3)cc21] 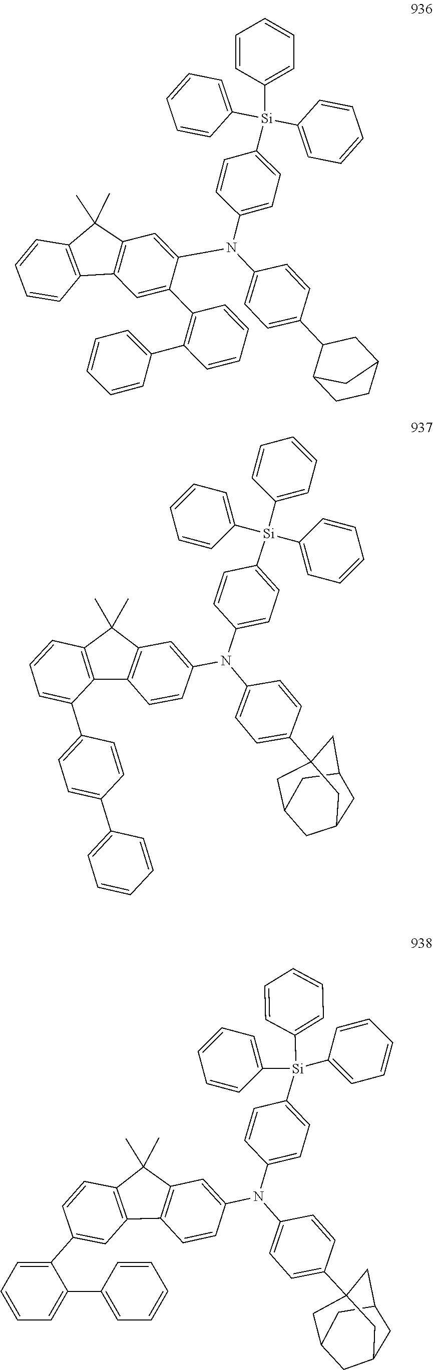 [SMILES: CC1(C)c2ccccc2-c2cc(-c3ccccc3-c3ccccc3)c(N(c3ccc(C4CC5CCC4C5)cc3)c3ccc([Si](c4ccccc4)(c4ccccc4)c4ccccc4)cc3)cc21 ; CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3ccc(-c4ccccc4)cc3)cccc21 ; CC1(C)c2ccc(-c3ccccc3-c3ccccc3)cc2-c2ccc(N(c3ccc(C45CC6CC(CC(C6)C4)C5)cc3)c3ccc([Si](c4ccccc4)(c4ccccc4)c4ccccc4)cc3)cc21] 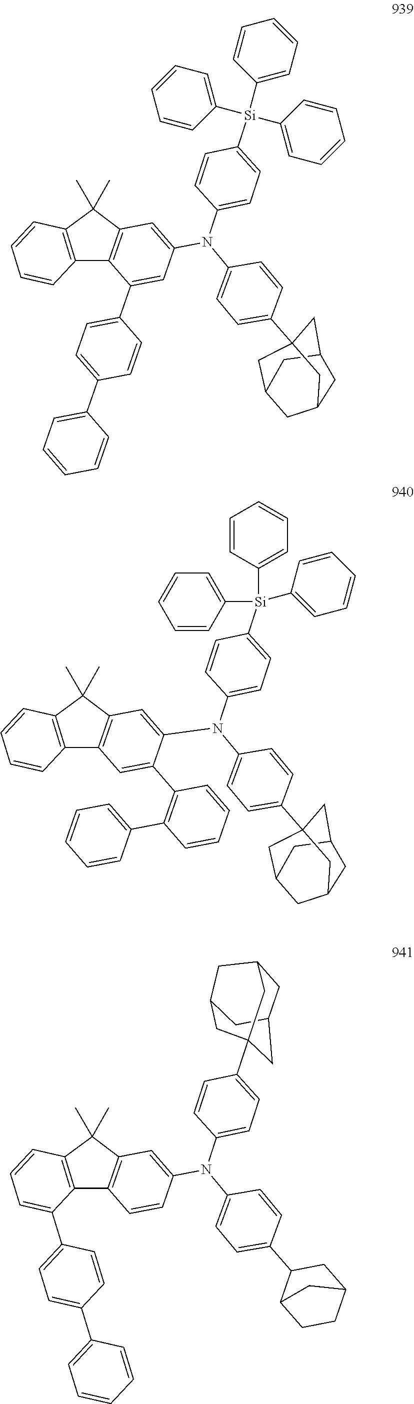 [SMILES: CC1(C)c2ccccc2-c2c(-c3ccc(-c4ccccc4)cc3)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3ccccc3-c3ccccc3)c(N(c3ccc(C45CC6CC(CC(C6)C4)C5)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C45CC6CC(CC(C6)C4)C5)cc3)ccc2-c2c(-c3ccc(-c4ccccc4)cc3)cccc21] 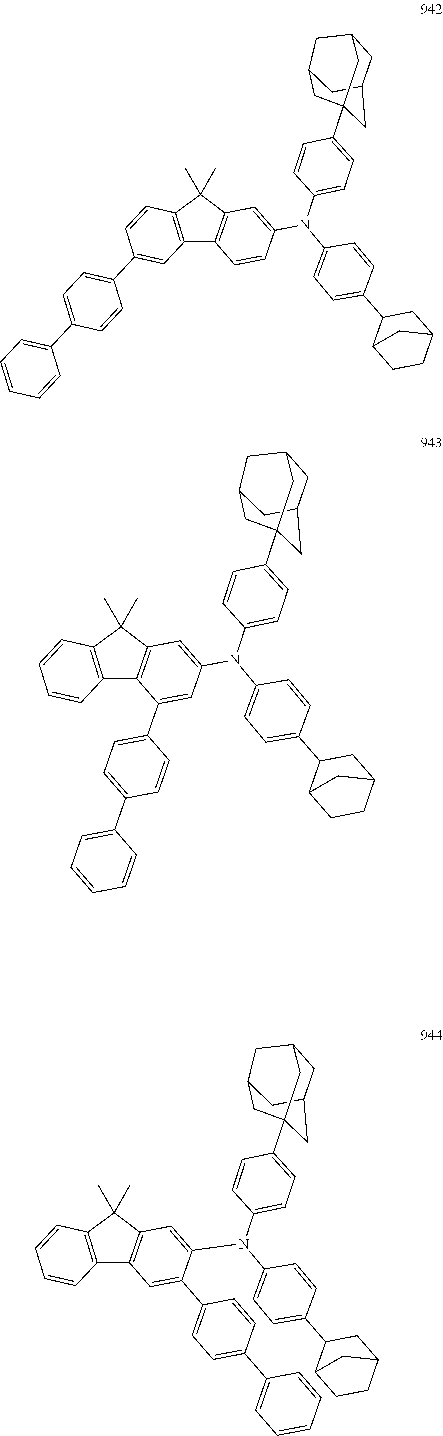 [SMILES: CC1(C)c2ccc(-c3ccc(-c4ccccc4)cc3)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3ccc(-c4ccccc4)cc3)cc(N(c3ccc(C4CC5CCC4C5)cc3)c3ccc(C45CC6CC(CC(C6)C4)C5)cc3)cc21 ; CC1(C)c2ccccc2-c2cc(-c3ccc(-c4ccccc4)cc3)c(N(c3ccc(C4CC5CCC4C5)cc3)c3ccc(C45CC6CC(CC(C6)C4)C5)cc3)cc21] 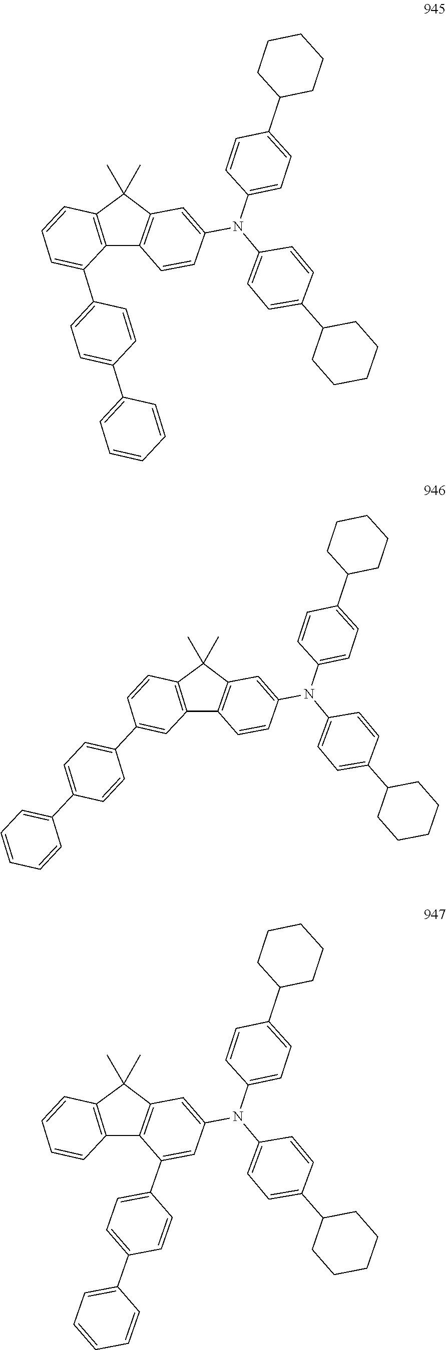 [SMILES: CC1(C)c2cc(N(c3ccc(C4CCCCC4)cc3)c3ccc(C4CCCCC4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc(C4CCCCC4)cc3)cc21 ; CC1(C)c2ccccc2-c2c(-c3ccc(-c4ccccc4)cc3)cc(N(c3ccc(C4CCCCC4)cc3)c3ccc(C4CCCCC4)cc3)cc21] 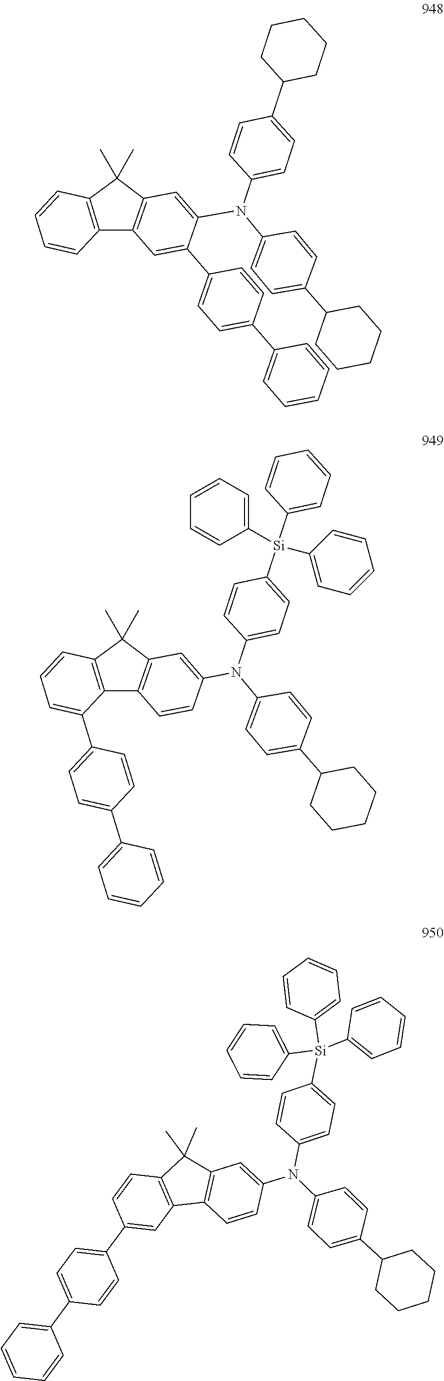 [SMILES: CC1(C)c2ccccc2-c2cc(-c3ccc(-c4ccccc4)cc3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccc(-c4ccccc4)cc3)cccc21 ; CC1(C)c2ccc(-c3ccc(-c4ccccc4)cc3)cc2-c2ccc(N(c3ccc(C4CCCCC4)cc3)c3ccc([Si](c4ccccc4)(c4ccccc4)c4ccccc4)cc3)cc21] 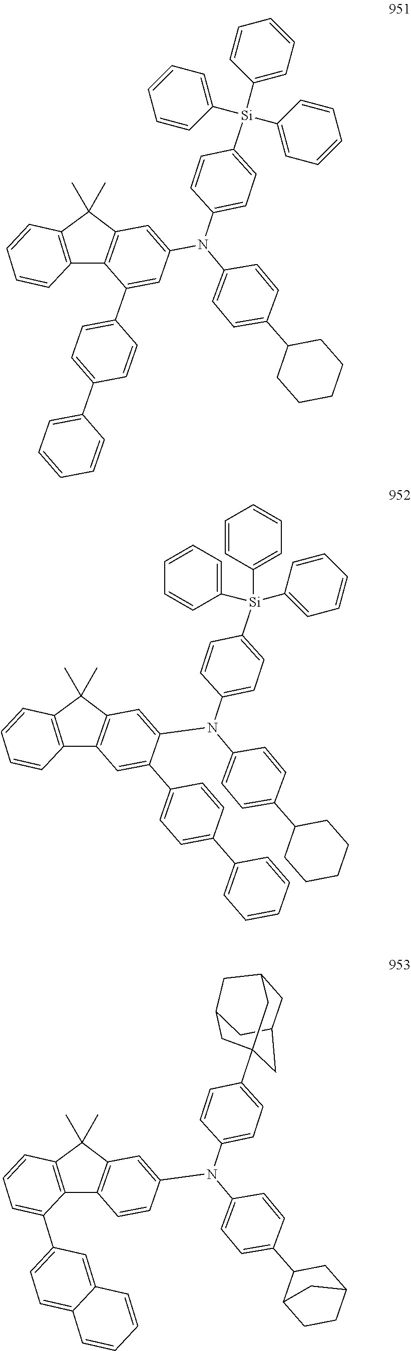 [SMILES: CC1(C)c2ccccc2-c2c(-c3ccc(-c4ccccc4)cc3)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3ccc(-c4ccccc4)cc3)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C45CC6CC(CC(C6)C4)C5)cc3)ccc2-c2c(-c3ccc4ccccc4c3)cccc21] 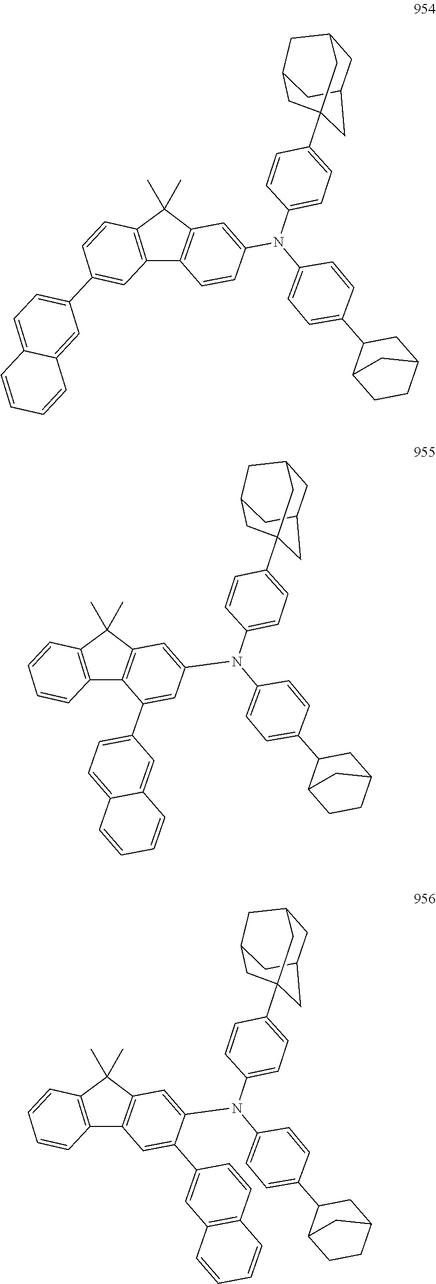 [SMILES: CC1(C)c2ccc(-c3ccc4ccccc4c3)cc2-c2ccc(N(c3ccc(C4CC5CCC4C5)cc3)c3ccc(C45CC6CC(CC(C6)C4)C5)cc3)cc21 ; CC1(C)c2ccccc2-c2c(-c3ccc4ccccc4c3)cc(N(c3ccc(C4CC5CCC4C5)cc3)c3ccc(C45CC6CC(CC(C6)C4)C5)cc3)cc21 ; CC1(C)c2ccccc2-c2cc(-c3ccc4ccccc4c3)c(N(c3ccc(C4CC5CCC4C5)cc3)c3ccc(C45CC6CC(CC(C6)C4)C5)cc3)cc21] 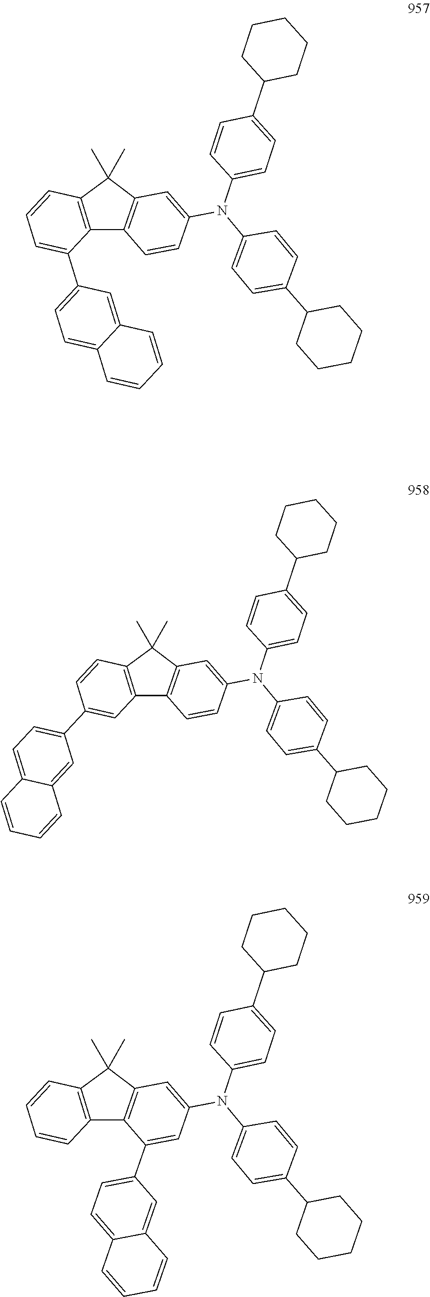 [SMILES: CC1(C)c2cc(N(c3ccc(C4CCCCC4)cc3)c3ccc(C4CCCCC4)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc(C4CCCCC4)cc3)cc21 ; CC1(C)c2ccccc2-c2c(-c3ccc4ccccc4c3)cc(N(c3ccc(C4CCCCC4)cc3)c3ccc(C4CCCCC4)cc3)cc21] 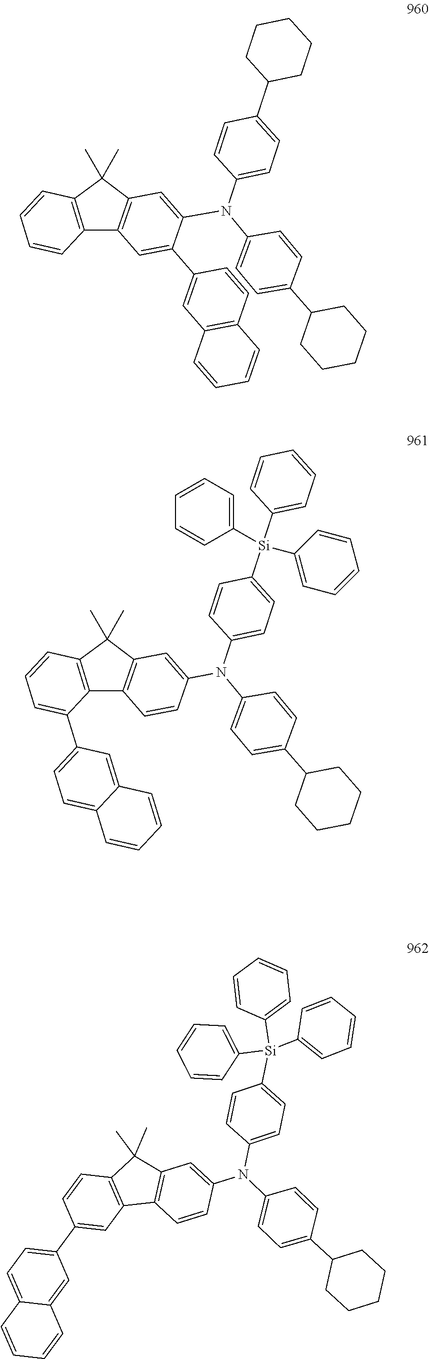 [SMILES: CC1(C)c2ccccc2-c2cc(-c3ccc4ccccc4c3)c(N(c3ccc(C4CCCCC4)cc3)c3ccc(C4CCCCC4)cc3)cc21 ; CC1(C)c2cc(N(c3ccc(C4CCCCC4)cc3)c3ccc([Si](c4ccccc4)(c4ccccc4)c4ccccc4)cc3)ccc2-c2c(-c3ccc4ccccc4c3)cccc21 ; CC1(C)c2ccc(-c3ccc4ccccc4c3)cc2-c2ccc(N(c3ccc(C4CCCCC4)cc3)c3ccc([Si](c4ccccc4)(c4ccccc4)c4ccccc4)cc3)cc21] 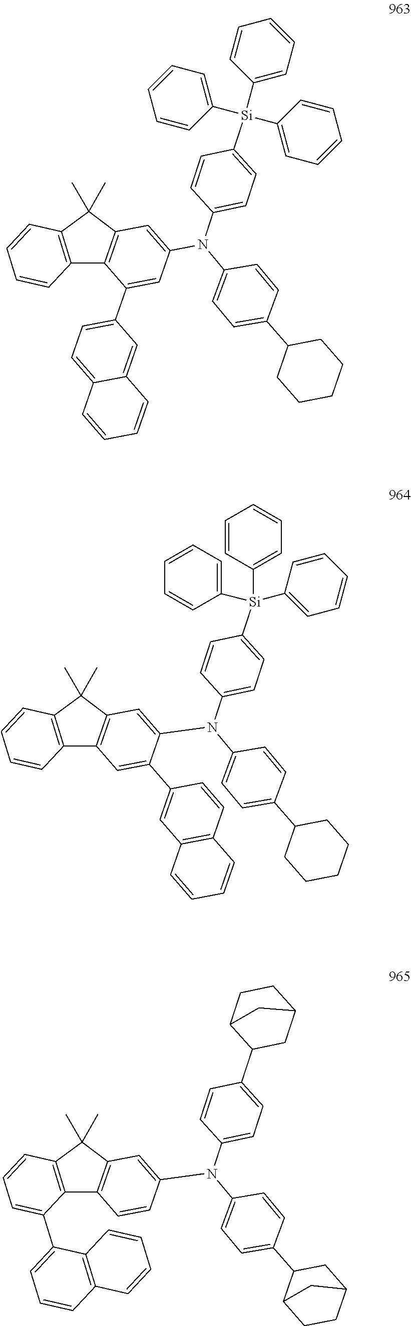 [SMILES: CC1(C)c2ccccc2-c2c(-c3ccc4ccccc4c3)cc(N(c3ccc(C4CCCCC4)cc3)c3ccc([Si](c4ccccc4)(c4ccccc4)c4ccccc4)cc3)cc21 ; CC1(C)c2ccccc2-c2cc(-c3ccc4ccccc4c3)c(N(c3ccc(C4CCCCC4)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C4CC5CCC4C5)cc3)ccc2-c2c(-c3cccc4ccccc34)cccc21] 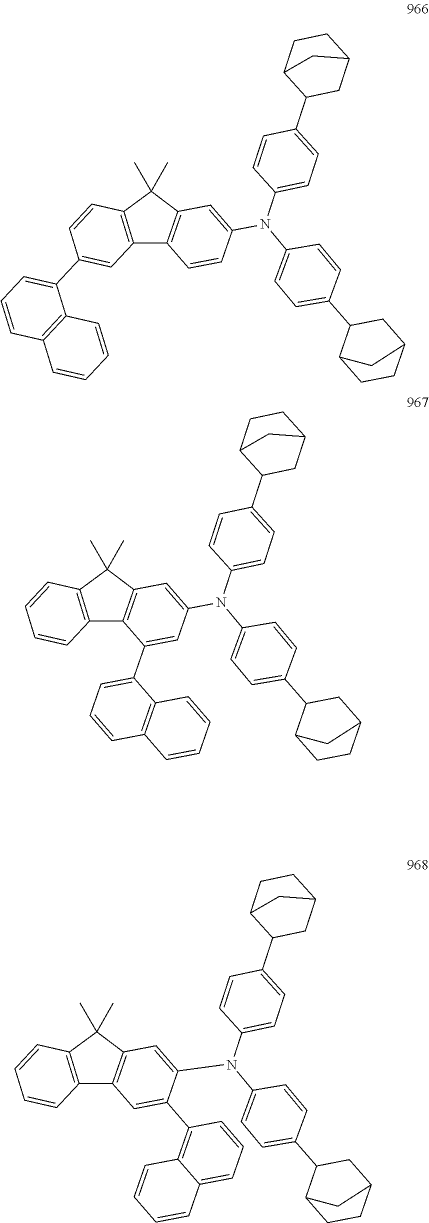 [SMILES: CC1(C)c2ccc(-c3cccc4ccccc34)cc2-c2ccc(N(c3ccc(C4CC5CCC4C5)cc3)c3ccc(C4CC5CCC4C5)cc3)cc21 ; CC1(C)c2ccccc2-c2c(-c3cccc4ccccc34)cc(N(c3ccc(C4CC5CCC4C5)cc3)c3ccc(C4CC5CCC4C5)cc3)cc21 ; CC1(C)c2ccccc2-c2cc(-c3cccc4ccccc34)c(N(c3ccc(C4CC5CCC4C5)cc3)c3ccc(C4CC5CCC4C5)cc3)cc21] 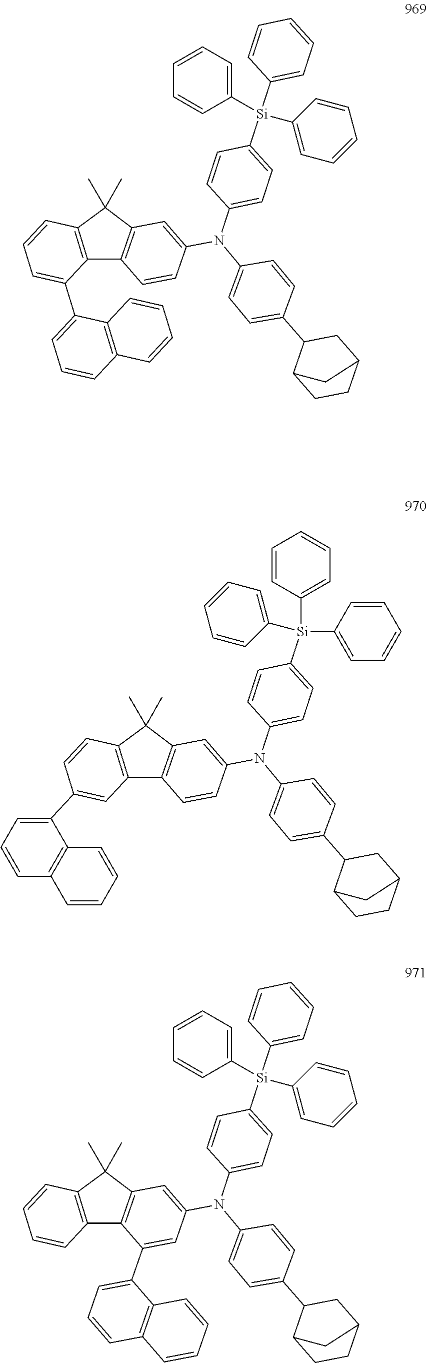 [SMILES: CC1(C)c2cc(N(c3ccc(C4CC5CCC4C5)cc3)c3ccc([Si](c4ccccc4)(c4ccccc4)c4ccccc4)cc3)ccc2-c2c(-c3cccc4ccccc34)cccc21 ; CC1(C)c2ccc(-c3cccc4ccccc34)cc2-c2ccc(N(c3ccc(C4CC5CCC4C5)cc3)c3ccc([Si](c4ccccc4)(c4ccccc4)c4ccccc4)cc3)cc21 ; CC1(C)c2ccccc2-c2c(-c3cccc4ccccc34)cc(N(c3ccc(C4CC5CCC4C5)cc3)c3ccc([Si](c4ccccc4)(c4ccccc4)c4ccccc4)cc3)cc21] 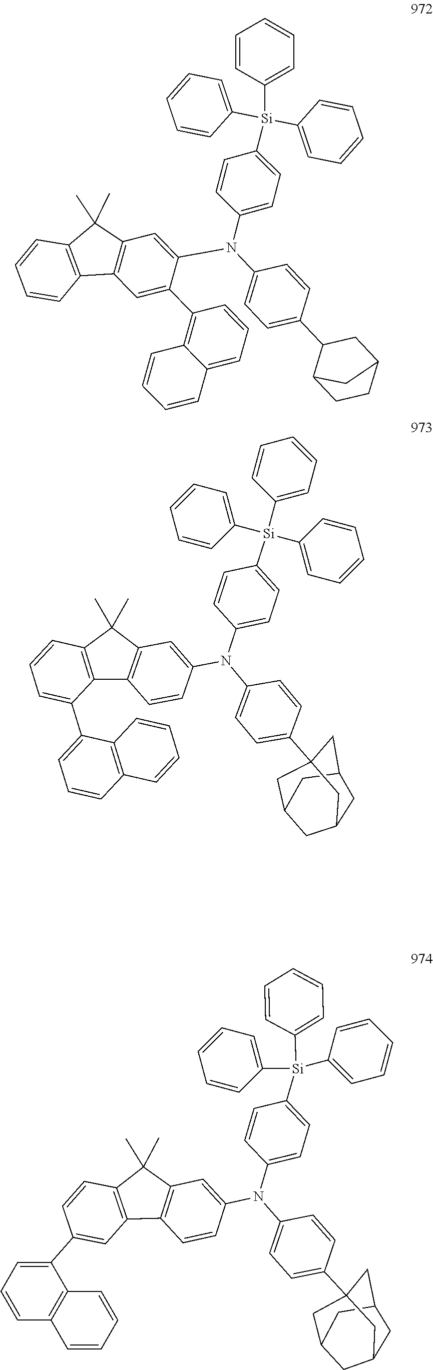 [SMILES: CC1(C)c2ccccc2-c2cc(-c3cccc4ccccc34)c(N(c3ccc(C4CC5CCC4C5)cc3)c3ccc([Si](c4ccccc4)(c4ccccc4)c4ccccc4)cc3)cc21 ; CC1(C)c2cc(N(c3ccc(C45CC6CC(CC(C6)C4)C5)cc3)c3ccc([Si](c4ccccc4)(c4ccccc4)c4ccccc4)cc3)ccc2-c2c(-c3cccc4ccccc34)cccc21 ; CC1(C)c2ccc(-c3cccc4ccccc34)cc2-c2ccc(N(c3ccc(C45CC6CC(CC(C6)C4)C5)cc3)c3ccc([Si](c4ccccc4)(c4ccccc4)c4ccccc4)cc3)cc21] 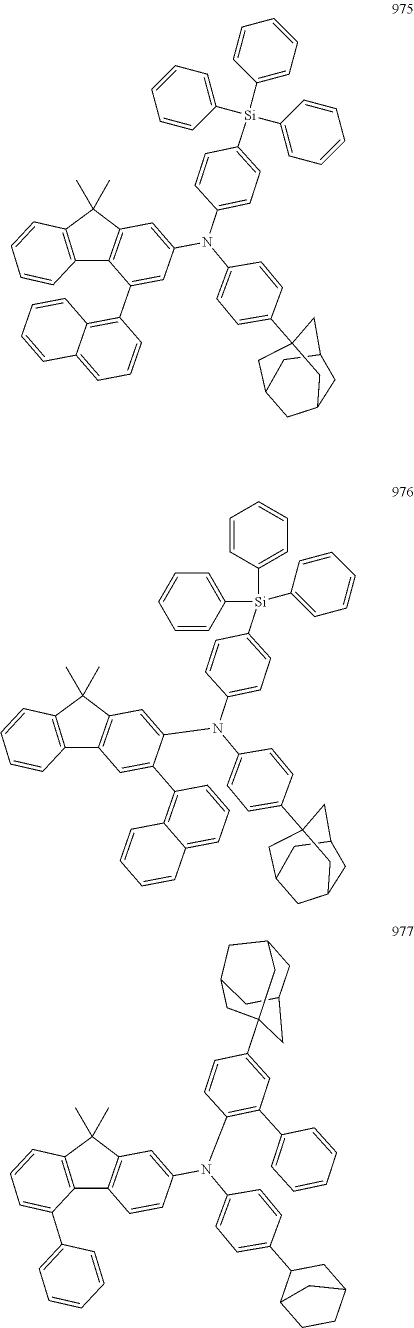 [SMILES: CC1(C)c2ccccc2-c2c(-c3cccc4ccccc34)cc(N(c3ccc(C45CC6CC(CC(C6)C4)C5)cc3)c3ccc([Si](c4ccccc4)(c4ccccc4)c4ccccc4)cc3)cc21 ; CC1(C)c2ccccc2-c2cc(-c3cccc4ccccc34)c(N(c3ccc(C45CC6CC(CC(C6)C4)C5)cc3)c3ccc([Si](c4ccccc4)(c4ccccc4)c4ccccc4)cc3)cc21 ; CC1(C)c2cc(N(c3ccc(C4CC5CCC4C5)cc3)c3ccc(C45CC6CC(CC(C6)C4)C5)cc3-c3ccccc3)ccc2-c2c(-c3ccccc3)cccc21] 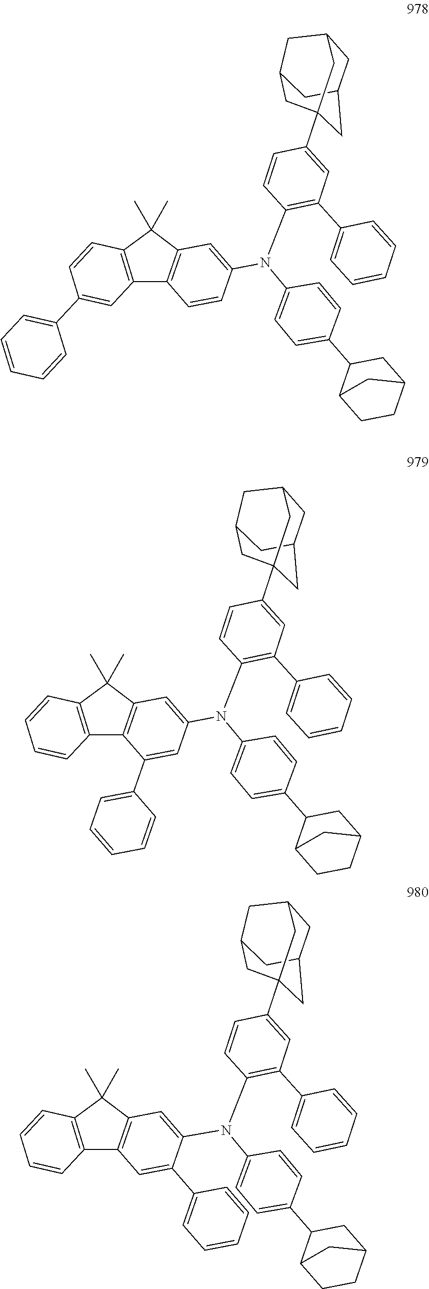 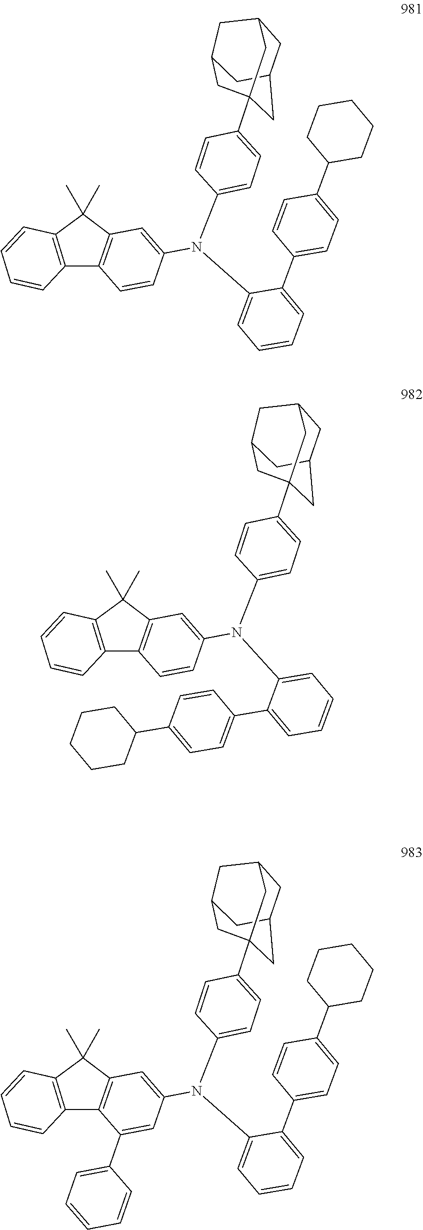 [SMILES: CC1(C)c2ccccc2-c2ccc(N(c3ccc(C45CC6CC(CC(C6)C4)C5)cc3)c3ccccc3-c3ccc(C4CCCCC4)cc3)cc21 ; CC1(C)c2ccccc2-c2ccc(N(c3ccc(C45CC6CC(CC(C6)C4)C5)cc3)c3ccccc3-c3ccc(C4CCCCC4)cc3)cc21 ; CC1(C)c2ccccc2-c2c(-c3ccccc3)cc(N(c3ccc(C45CC6CC(CC(C6)C4)C5)cc3)c3ccccc3-c3ccc(C4CCCCC4)cc3)cc21] 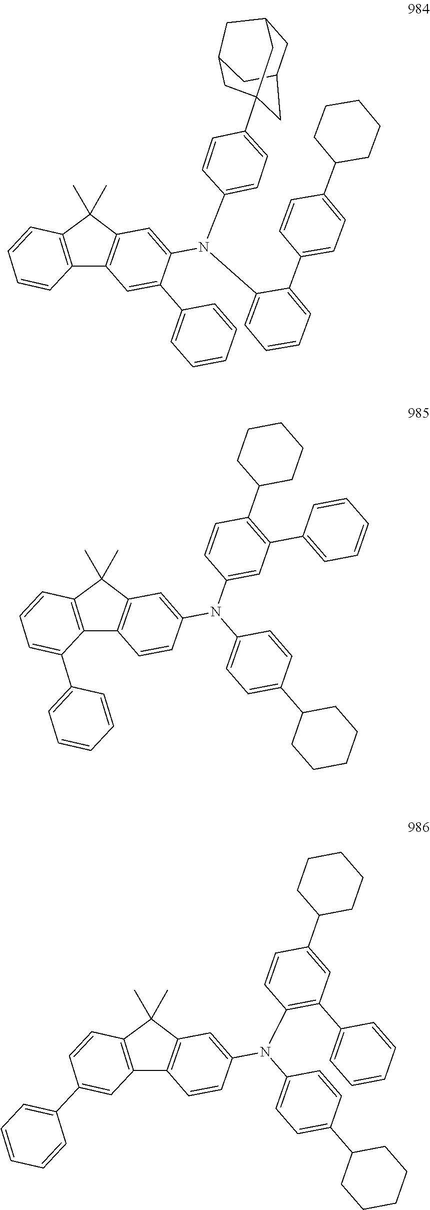 [SMILES: CC1(C)c2ccccc2-c2cc(-c3ccccc3)c(N(c3ccc(C45CC6CC(CC(C6)C4)C5)cc3)c3ccccc3-c3ccc(C4CCCCC4)cc3)cc21 ; CC1(C)c2cc(N(c3ccc(C4CCCCC4)cc3)c3ccc(C4CCCCC4)c(-c4ccccc4)c3)ccc2-c2c(-c3ccccc3)cccc21 ; CC1(C)c2ccc(-c3ccccc3)cc2-c2ccc(N(c3ccc(C4CCCCC4)cc3)c3ccc(C4CCCCC4)cc3-c3ccccc3)cc21] 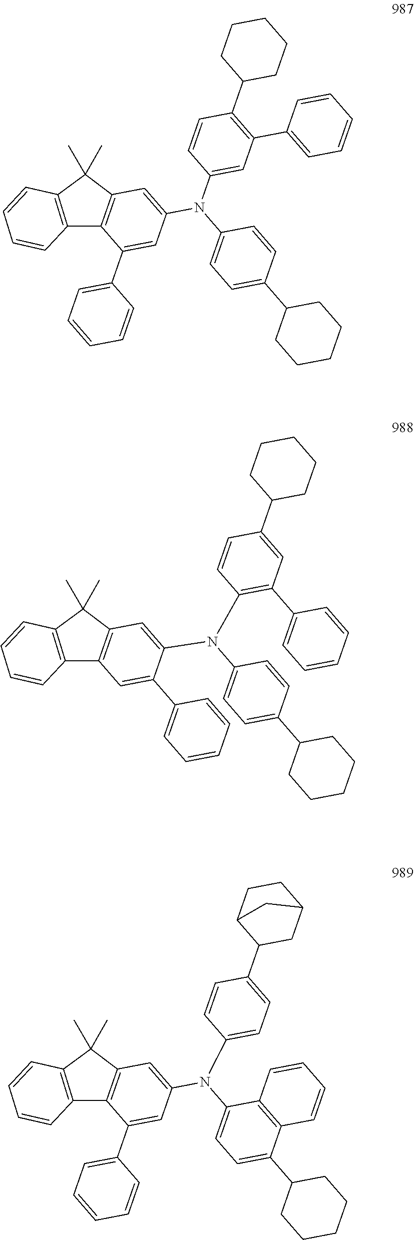 [SMILES: CC1(C)c2ccccc2-c2c(-c3ccccc3)cc(N(c3ccc(C4CCCCC4)cc3)c3ccc(C4CCCCC4)c(-c4ccccc4)c3)cc21 ; CC1(C)c2ccccc2-c2cc(-c3ccccc3)c(N(c3ccc(C4CCCCC4)cc3)c3ccc(C4CCCCC4)cc3-c3ccccc3)cc21 ; CC1(C)c2ccccc2-c2c(-c3ccccc3)cc(N(c3ccc(C4CC5CCC4C5)cc3)c3ccc(C4CCCCC4)c4ccccc34)cc21] 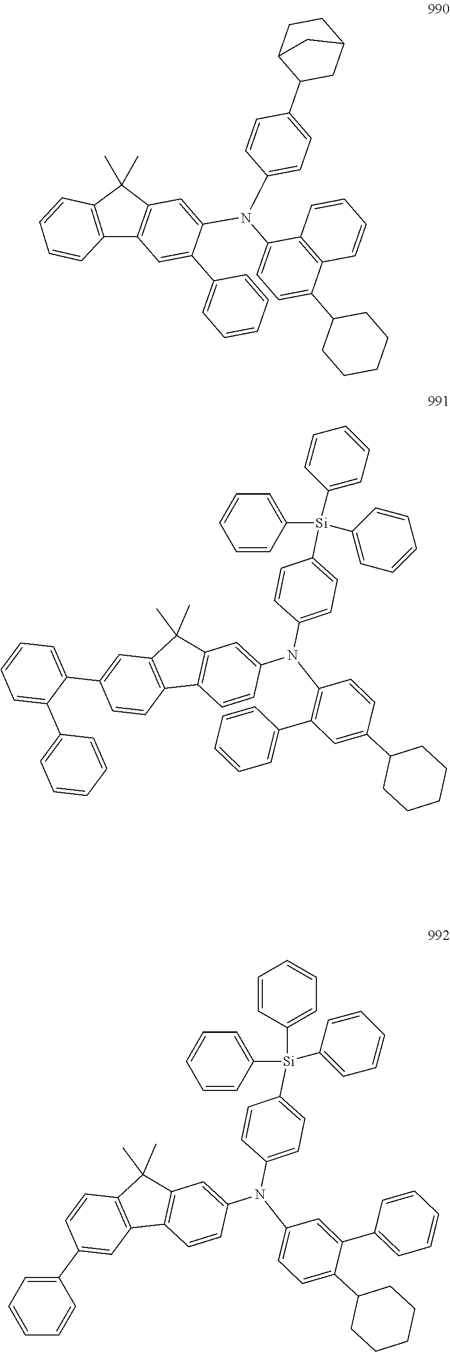 [SMILES: CC1(C)c2ccccc2-c2cc(-c3ccccc3)c(N(c3ccc(C4CC5CCC4C5)cc3)c3ccc(C4CCCCC4)c4ccccc34)cc21 ; CC1(C)c2cc(-c3ccccc3-c3ccccc3)ccc2-c2ccc(N(c3ccc([Si](c4ccccc4)(c4ccccc4)c4ccccc4)cc3)c3ccc(C4CCCCC4)cc3-c3ccccc3)cc21 ; CC1(C)c2ccc(-c3ccccc3)cc2-c2ccc(N(c3ccc([Si](c4ccccc4)(c4ccccc4)c4ccccc4)cc3)c3ccc(C4CCCCC4)c(-c4ccccc4)c3)cc21] 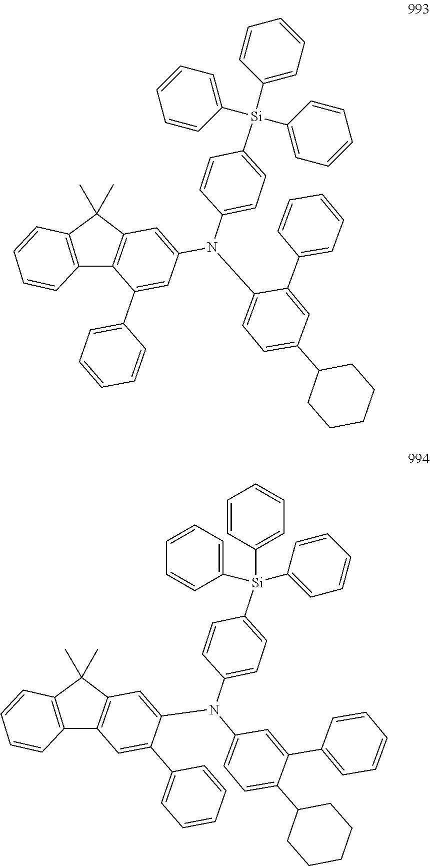 [SMILES: CC1(C)c2ccccc2-c2c(-c3ccccc3)cc(N(c3ccc([Si](c4ccccc4)(c4ccccc4)c4ccccc4)cc3)c3ccc(C4CCCCC4)cc3-c3ccccc3)cc21 ; CC1(C)c2ccccc2-c2cc(-c3ccccc3)c(N(c3ccc([Si](c4ccccc4)(c4ccccc4)c4ccccc4)cc3)c3ccc(C4CCCCC4)c(-c4ccccc4)c3)cc21] 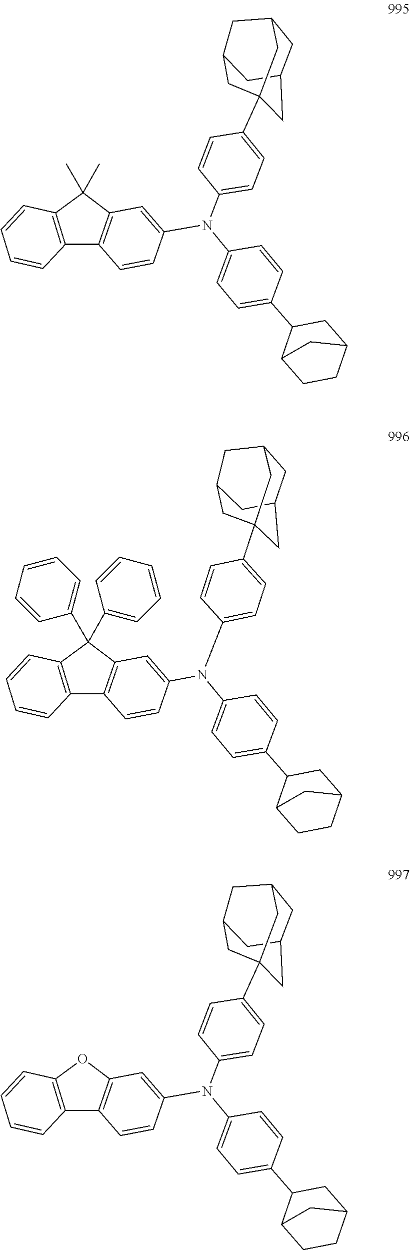 [SMILES: CC1(C)c2ccccc2-c2ccc(N(c3ccc(C4CC5CCC4C5)cc3)c3ccc(C45CC6CC(CC(C6)C4)C5)cc3)cc21 ; c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1 ; c1ccc2c(c1)oc1cc(N(c3ccc(C4CC5CCC4C5)cc3)c3ccc(C45CC6CC(CC(C6)C4)C5)cc3)ccc12] 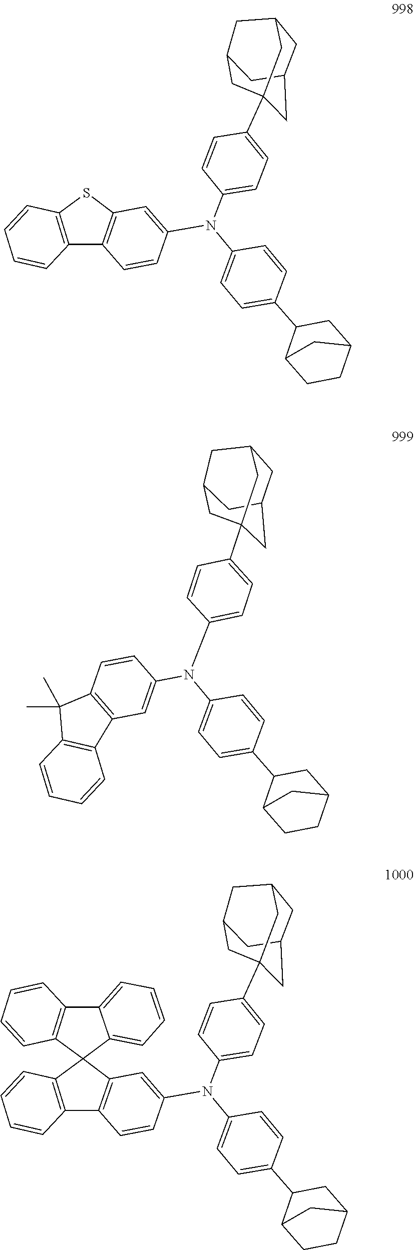 [SMILES: c1ccc2c(c1)sc1cc(N(c3ccc(C4CC5CCC4C5)cc3)c3ccc(C45CC6CC(CC(C6)C4)C5)cc3)ccc12 ; CC1(C)c2ccccc2-c2cc(N(c3ccc(C4CC5CCC4C5)cc3)c3ccc(C45CC6CC(CC(C6)C4)C5)cc3)ccc21 ; c1ccc2c(c1)-c1ccccc1C21c2ccccc2-c2ccc(N(c3ccc(C4CC5CCC4C5)cc3)c3ccc(C45CC6CC(CC(C6)C4)C5)cc3)cc21] 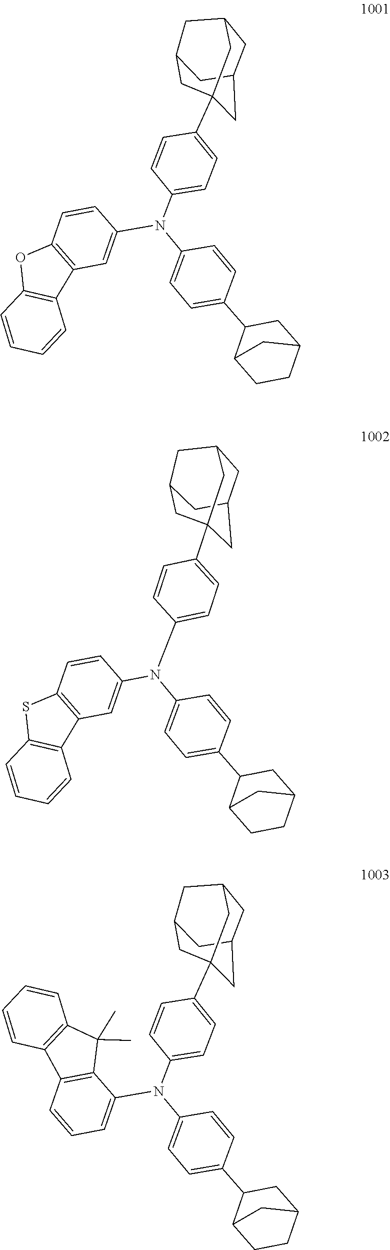 [SMILES: c1ccc2c(c1)oc1ccc(N(c3ccc(C4CC5CCC4C5)cc3)c3ccc(C45CC6CC(CC(C6)C4)C5)cc3)cc12 ; c1ccc2c(c1)sc1ccc(N(c3ccc(C4CC5CCC4C5)cc3)c3ccc(C45CC6CC(CC(C6)C4)C5)cc3)cc12 ; CC1(C)c2ccccc2-c2cccc(N(c3ccc(C4CC5CCC4C5)cc3)c3ccc(C45CC6CC(CC(C6)C4)C5)cc3)c21] 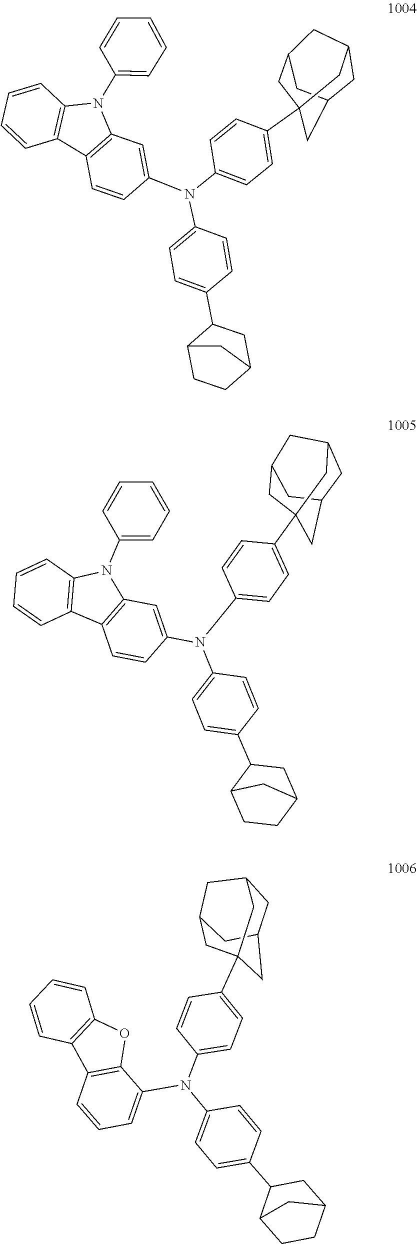 [SMILES: c1ccc(-n2c3ccccc3c3ccc(N(c4ccc(C5CC6CCC5C6)cc4)c4ccc(C56CC7CC(CC(C7)C5)C6)cc4)cc32)cc1 ; c1ccc(-n2c3ccccc3c3ccc(N(c4ccc(C5CC6CCC5C6)cc4)c4ccc(C56CC7CC(CC(C7)C5)C6)cc4)cc32)cc1 ; c1ccc2c(c1)oc1c(N(c3ccc(C4CC5CCC4C5)cc3)c3ccc(C45CC6CC(CC(C6)C4)C5)cc3)cccc12] 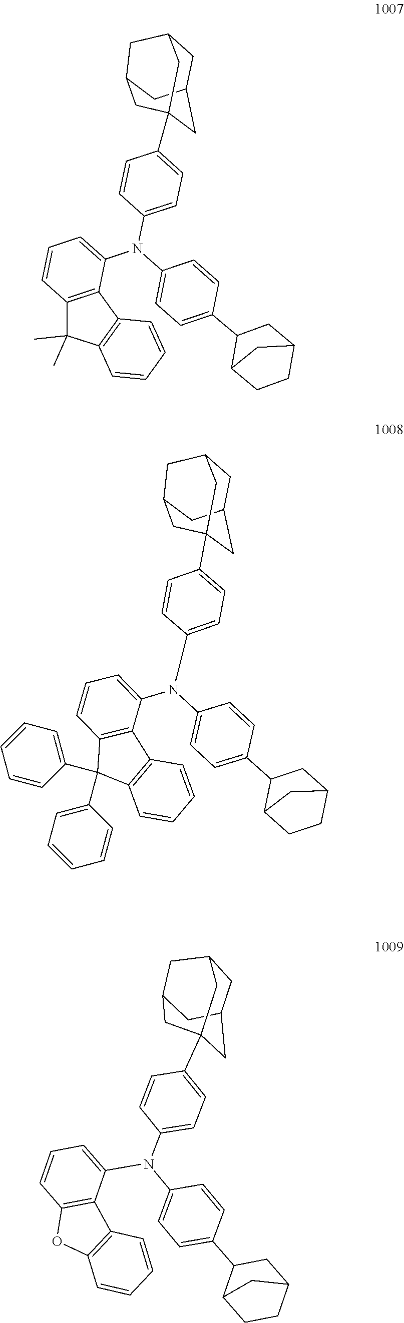 [SMILES: CC1(C)c2ccccc2-c2c(N(c3ccc(C4CC5CCC4C5)cc3)c3ccc(C45CC6CC(CC(C6)C4)C5)cc3)cccc21 ; c1ccc(C2(c3ccccc3)c3ccccc3-c3c(N(c4ccc(C5CC6CCC5C6)cc4)c4ccc(C56CC7CC(CC(C7)C5)C6)cc4)cccc32)cc1 ; c1ccc2c(c1)oc1cccc(N(c3ccc(C4CC5CCC4C5)cc3)c3ccc(C45CC6CC(CC(C6)C4)C5)cc3)c12] 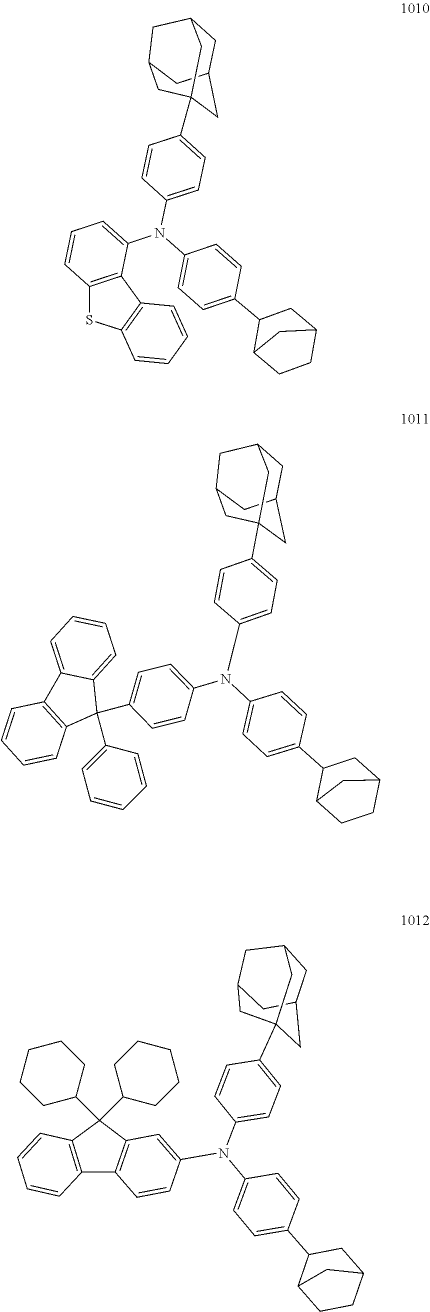 [SMILES: c1ccc2c(c1)sc1cccc(N(c3ccc(C4CC5CCC4C5)cc3)c3ccc(C45CC6CC(CC(C6)C4)C5)cc3)c12 ; c1ccc(C2(c3ccc(N(c4ccc(C5CC6CCC5C6)cc4)c4ccc(C56CC7CC(CC(C7)C5)C6)cc4)cc3)c3ccccc3-c3ccccc32)cc1 ; c1ccc2c(c1)-c1ccc(N(c3ccc(C4CC5CCC4C5)cc3)c3ccc(C45CC6CC(CC(C6)C4)C5)cc3)cc1C2(C1CCCCC1)C1CCCCC1] 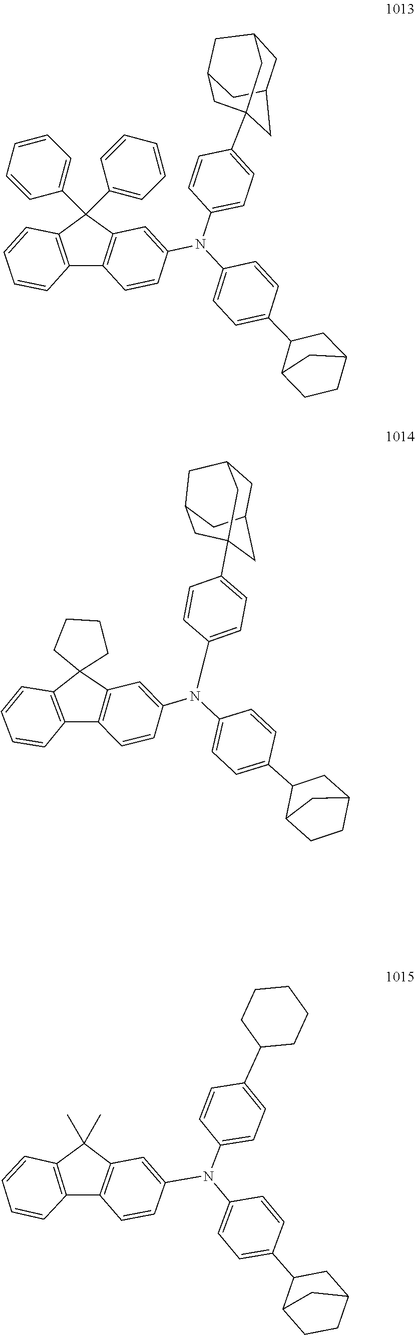 [SMILES: c1ccc(C2(c3ccccc3)c3ccccc3-c3ccc(N(c4ccc(C5CC6CCC5C6)cc4)c4ccc(C56CC7CC(CC(C7)C5)C6)cc4)cc32)cc1 ; c1ccc2c(c1)-c1ccc(N(c3ccc(C4CC5CCC4C5)cc3)c3ccc(C45CC6CC(CC(C6)C4)C5)cc3)cc1C21CCCC1 ; CC1(C)c2ccccc2-c2ccc(N(c3ccc(C4CCCCC4)cc3)c3ccc(C4CC5CCC4C5)cc3)cc21] 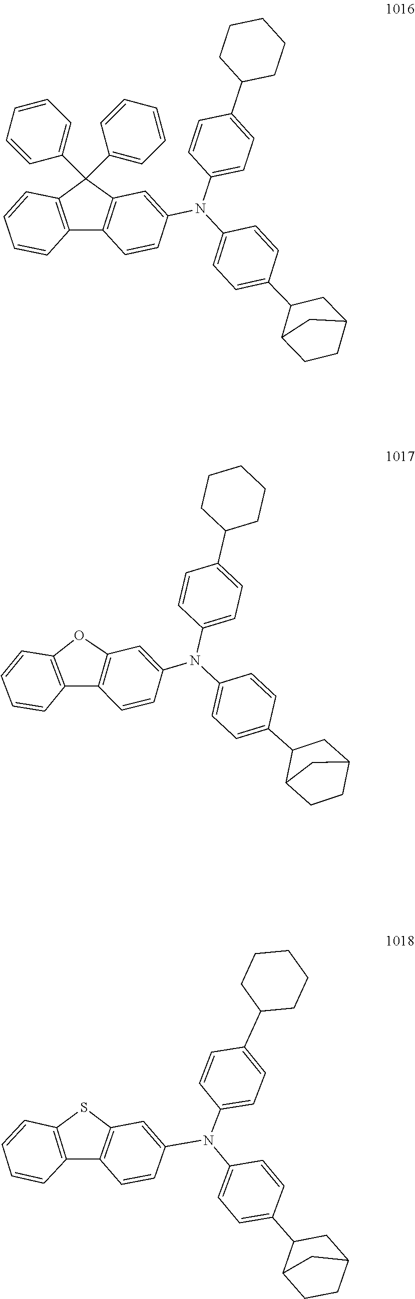 [SMILES: c1ccc(C2(c3ccccc3)c3ccccc3-c3ccc(N(c4ccc(C5CCCCC5)cc4)c4ccc(C5CC6CCC5C6)cc4)cc32)cc1 ; c1ccc2c(c1)oc1cc(N(c3ccc(C4CCCCC4)cc3)c3ccc(C4CC5CCC4C5)cc3)ccc12 ; c1ccc2c(c1)sc1cc(N(c3ccc(C4CCCCC4)cc3)c3ccc(C4CC5CCC4C5)cc3)ccc12] 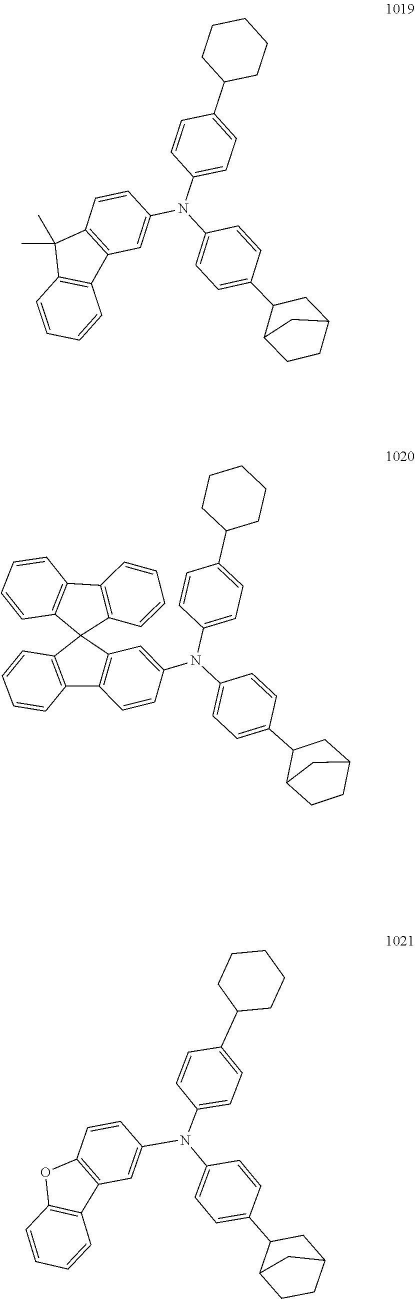 [SMILES: CC1(C)c2ccccc2-c2cc(N(c3ccc(C4CCCCC4)cc3)c3ccc(C4CC5CCC4C5)cc3)ccc21 ; c1ccc2c(c1)-c1ccccc1C21c2ccccc2-c2ccc(N(c3ccc(C4CCCCC4)cc3)c3ccc(C4CC5CCC4C5)cc3)cc21 ; c1ccc2c(c1)oc1ccc(N(c3ccc(C4CCCCC4)cc3)c3ccc(C4CC5CCC4C5)cc3)cc12] 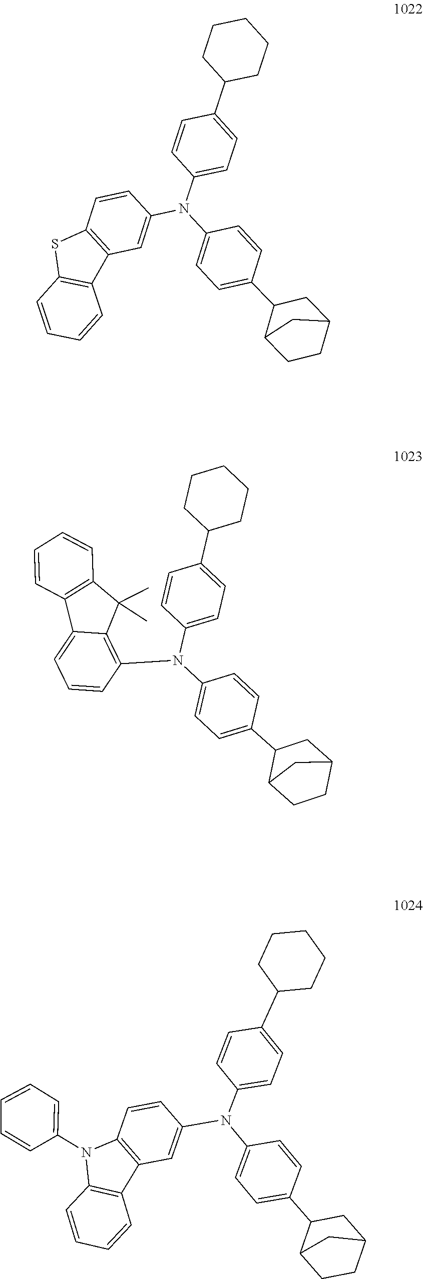 [SMILES: c1ccc2c(c1)sc1ccc(N(c3ccc(C4CCCCC4)cc3)c3ccc(C4CC5CCC4C5)cc3)cc12 ; CC1(C)c2ccccc2-c2cccc(N(c3ccc(C4CCCCC4)cc3)c3ccc(C4CC5CCC4C5)cc3)c21 ; c1ccc(-n2c3ccccc3c3cc(N(c4ccc(C5CCCCC5)cc4)c4ccc(C5CC6CCC5C6)cc4)ccc32)cc1] 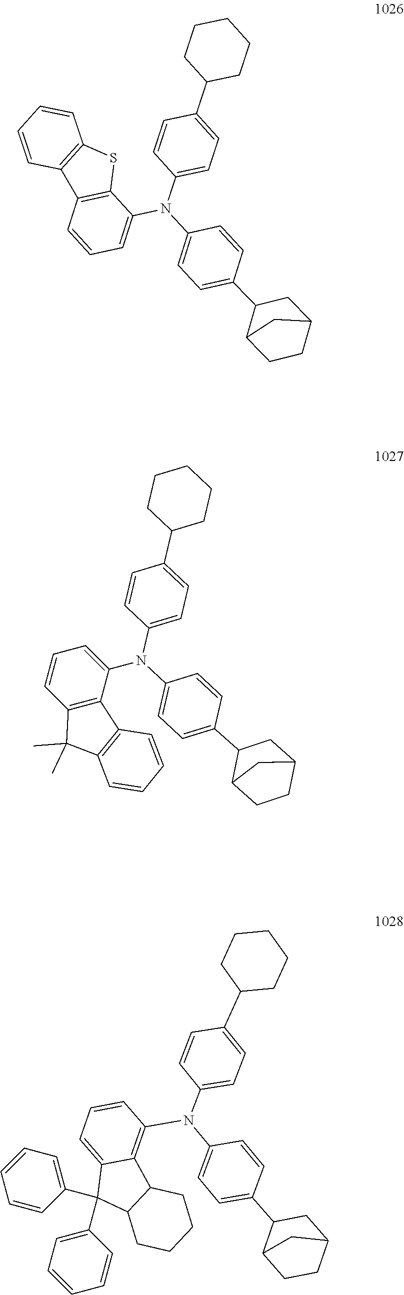 [SMILES: c1ccc2c(c1)sc1c(N(c3ccc(C4CCCCC4)cc3)c3ccc(C4CC5CCC4C5)cc3)cccc12 ; CC1(C)c2ccccc2-c2c(N(c3ccc(C4CCCCC4)cc3)c3ccc(C4CC5CCC4C5)cc3)cccc21 ; c1ccc(C2(c3ccccc3)c3cccc(N(c4ccc(C5CCCCC5)cc4)c4ccc(C5CC6CCC5C6)cc4)c3C3CCCCC32)cc1] 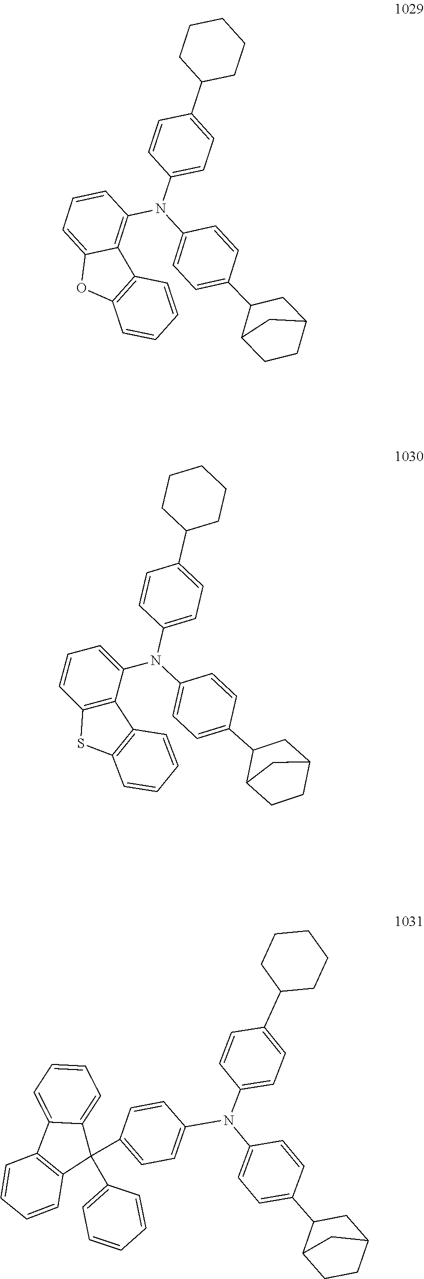 [SMILES: c1ccc2c(c1)oc1cccc(N(c3ccc(C4CCCCC4)cc3)c3ccc(C4CC5CCC4C5)cc3)c12 ; c1ccc2c(c1)sc1cccc(N(c3ccc(C4CCCCC4)cc3)c3ccc(C4CC5CCC4C5)cc3)c12 ; c1ccc(C2(c3ccc(N(c4ccc(C5CCCCC5)cc4)c4ccc(C5CC6CCC5C6)cc4)cc3)c3ccccc3-c3ccccc32)cc1] 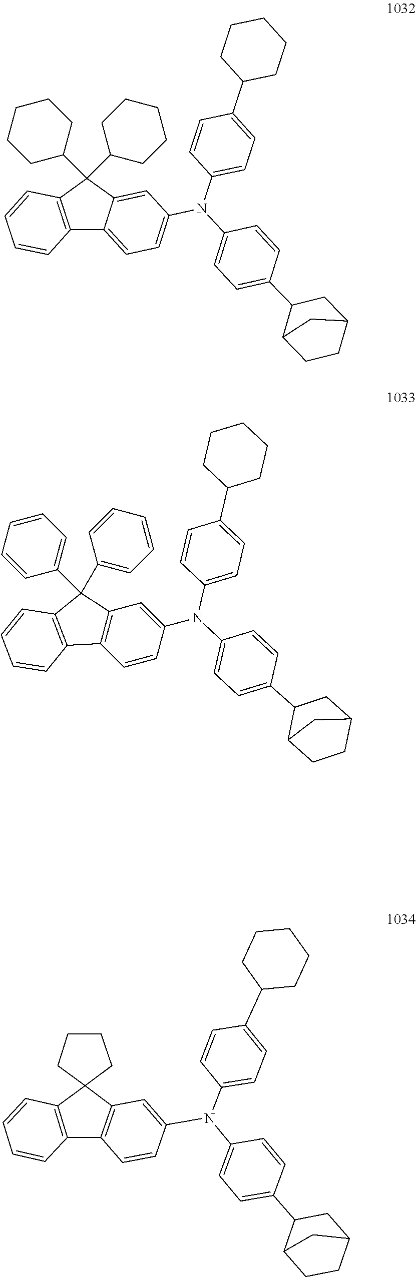 [SMILES: c1ccc2c(c1)-c1ccc(N(c3ccc(C4CCCCC4)cc3)c3ccc(C4CC5CCC4C5)cc3)cc1C2(C1CCCCC1)C1CCCCC1 ; c1ccc(C2(c3ccccc3)c3ccccc3-c3ccc(N(c4ccc(C5CCCCC5)cc4)c4ccc(C5CC6CCC5C6)cc4)cc32)cc1 ; c1ccc2c(c1)-c1ccc(N(c3ccc(C4CCCCC4)cc3)c3ccc(C4CC5CCC4C5)cc3)cc1C21CCCC1] 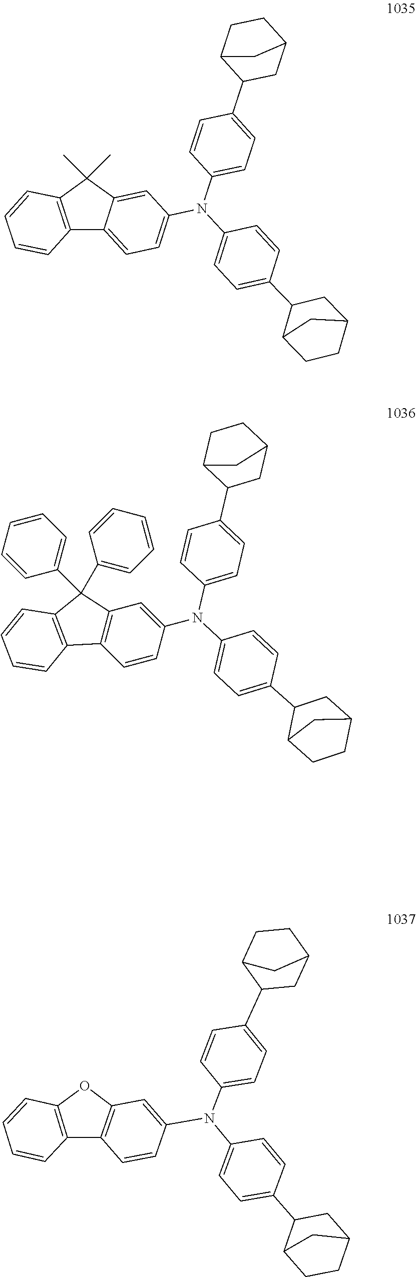 [SMILES: CC1(C)c2ccccc2-c2ccc(N(c3ccc(C4CC5CCC4C5)cc3)c3ccc(C4CC5CCC4C5)cc3)cc21 ; c1ccc(C2(c3ccccc3)c3ccccc3-c3ccc(N(c4ccc(C5CC6CCC5C6)cc4)c4ccc(C5CC6CCC5C6)cc4)cc32)cc1 ; c1ccc2c(c1)oc1cc(N(c3ccc(C4CC5CCC4C5)cc3)c3ccc(C4CC5CCC4C5)cc3)ccc12] 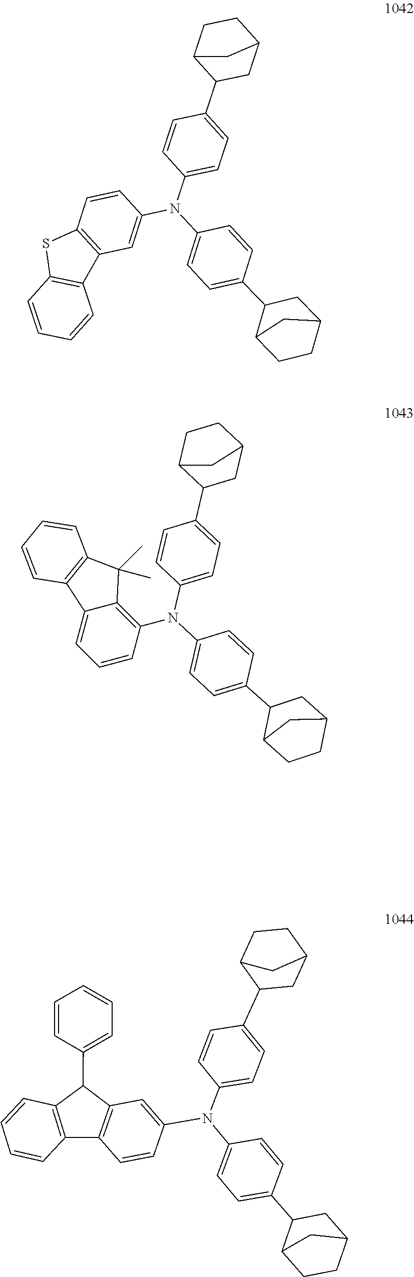 [SMILES: c1ccc2c(c1)sc1ccc(N(c3ccc(C4CC5CCC4C5)cc3)c3ccc(C4CC5CCC4C5)cc3)cc12 ; CC1(C)c2ccccc2-c2cccc(N(c3ccc(C4CC5CCC4C5)cc3)c3ccc(C4CC5CCC4C5)cc3)c21 ; c1ccc(C2c3ccccc3-c3ccc(N(c4ccc(C5CC6CCC5C6)cc4)c4ccc(C5CC6CCC5C6)cc4)cc32)cc1] 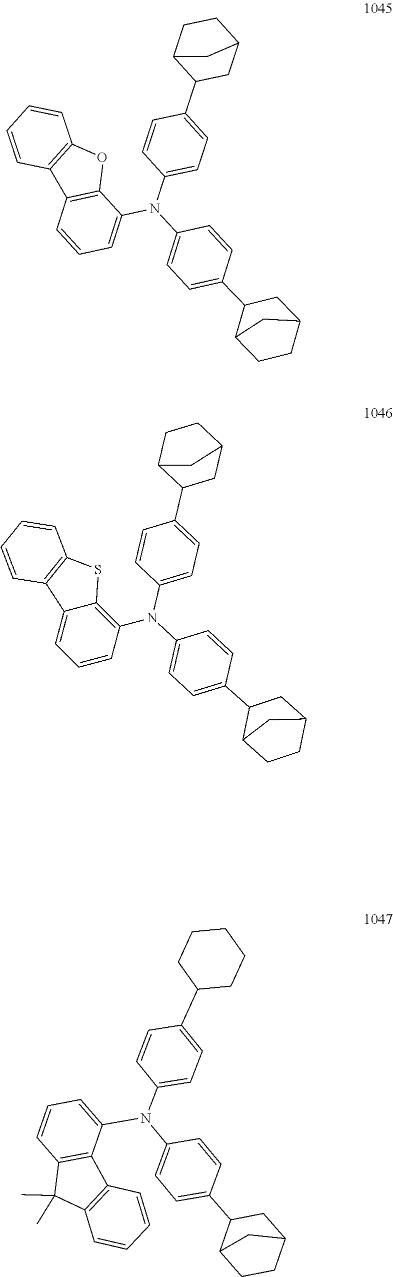 [SMILES: c1ccc2c(c1)oc1c(N(c3ccc(C4CC5CCC4C5)cc3)c3ccc(C4CC5CCC4C5)cc3)cccc12 ; c1ccc2c(c1)sc1c(N(c3ccc(C4CC5CCC4C5)cc3)c3ccc(C4CC5CCC4C5)cc3)cccc12 ; CC1(C)c2ccccc2-c2c(N(c3ccc(C4CCCCC4)cc3)c3ccc(C4CC5CCC4C5)cc3)cccc21] 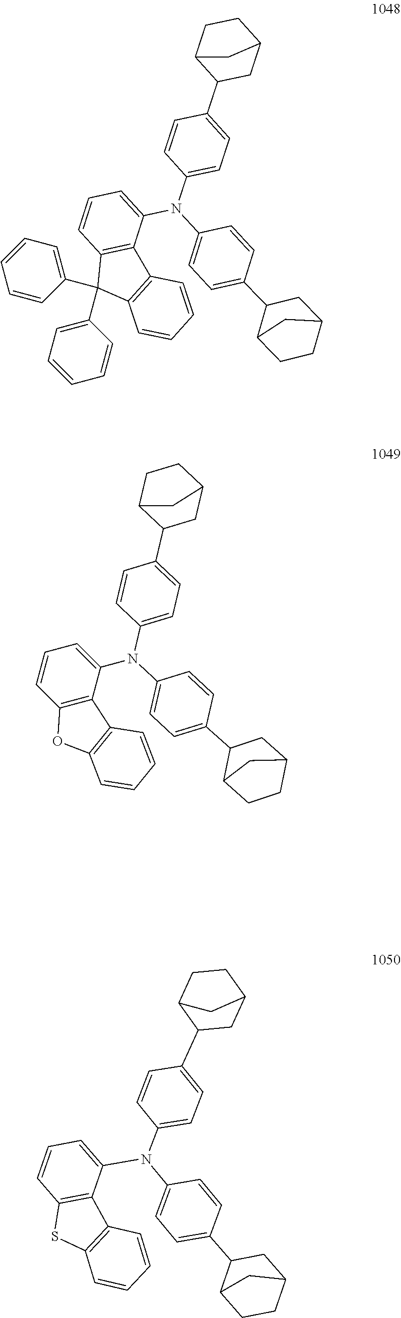 [SMILES: c1ccc(C2(c3ccccc3)c3ccccc3-c3c(N(c4ccc(C5CC6CCC5C6)cc4)c4ccc(C5CC6CCC5C6)cc4)cccc32)cc1 ; c1ccc2c(c1)oc1cccc(N(c3ccc(C4CC5CCC4C5)cc3)c3ccc(C4CC5CCC4C5)cc3)c12 ; c1ccc2c(c1)sc1cccc(N(c3ccc(C4CC5CCC4C5)cc3)c3ccc(C4CC5CCC4C5)cc3)c12] 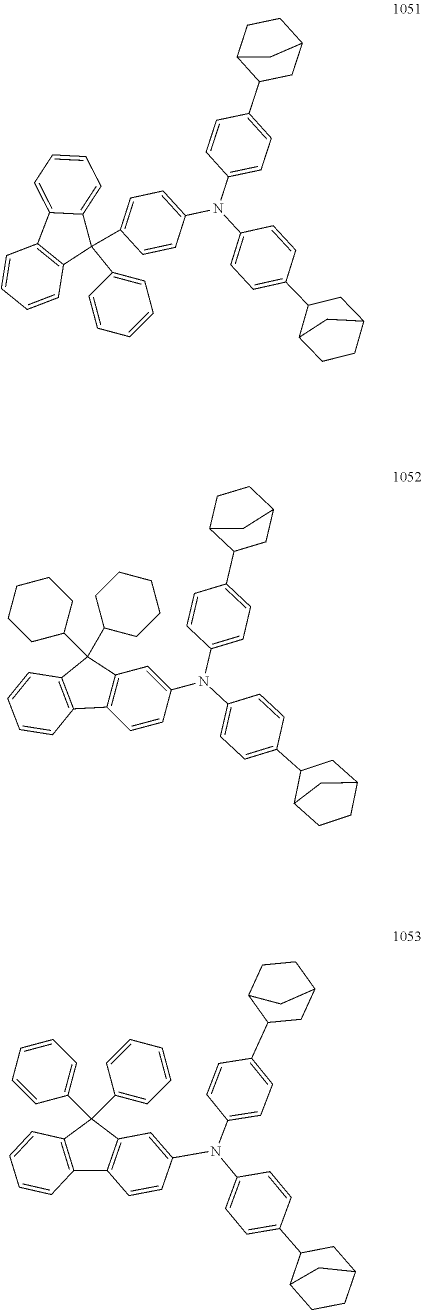 [SMILES: c1ccc(C2(c3ccc(N(c4ccc(C5CC6CCC5C6)cc4)c4ccc(C5CC6CCC5C6)cc4)cc3)c3ccccc3-c3ccccc32)cc1 ; c1ccc2c(c1)-c1ccc(N(c3ccc(C4CC5CCC4C5)cc3)c3ccc(C4CC5CCC4C5)cc3)cc1C2(C1CCCCC1)C1CCCCC1 ; c1ccc(C2(c3ccccc3)c3ccccc3-c3ccc(N(c4ccc(C5CC6CCC5C6)cc4)c4ccc(C5CC6CCC5C6)cc4)cc32)cc1] 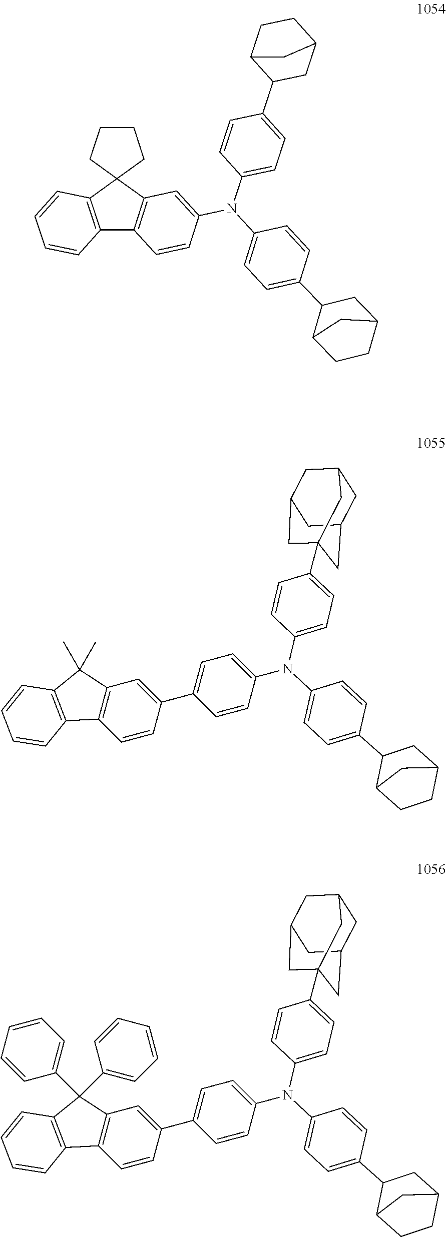 [SMILES: c1ccc2c(c1)-c1ccc(N(c3ccc(C4CC5CCC4C5)cc3)c3ccc(C4CC5CCC4C5)cc3)cc1C21CCCC1 ; CC1(C)c2ccccc2-c2ccc(-c3ccc(N(c4ccc(C5CC6CCC5C6)cc4)c4ccc(C56CC7CC(CC(C7)C5)C6)cc4)cc3)cc21 ; c1ccc(C2(c3ccccc3)c3ccccc3-c3ccc(-c4ccc(N(c5ccc(C6CC7CCC6C7)cc5)c5ccc(C67CC8CC(CC(C8)C6)C7)cc5)cc4)cc32)cc1] 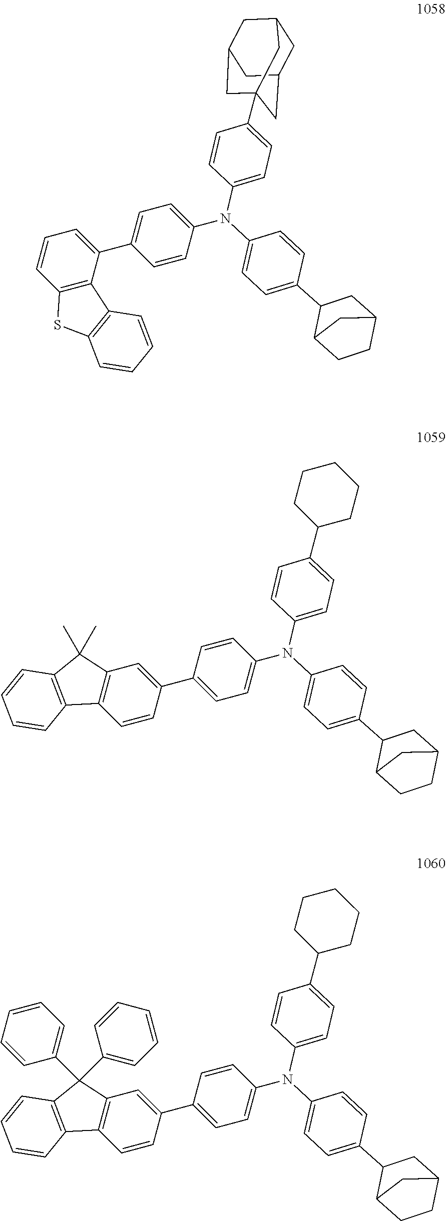 [SMILES: c1ccc2c(c1)sc1cccc(-c3ccc(N(c4ccc(C5CC6CCC5C6)cc4)c4ccc(C56CC7CC(CC(C7)C5)C6)cc4)cc3)c12 ; CC1(C)c2ccccc2-c2ccc(-c3ccc(N(c4ccc(C5CCCCC5)cc4)c4ccc(C5CC6CCC5C6)cc4)cc3)cc21 ; c1ccc(C2(c3ccccc3)c3ccccc3-c3ccc(-c4ccc(N(c5ccc(C6CCCCC6)cc5)c5ccc(C6CC7CCC6C7)cc5)cc4)cc32)cc1] 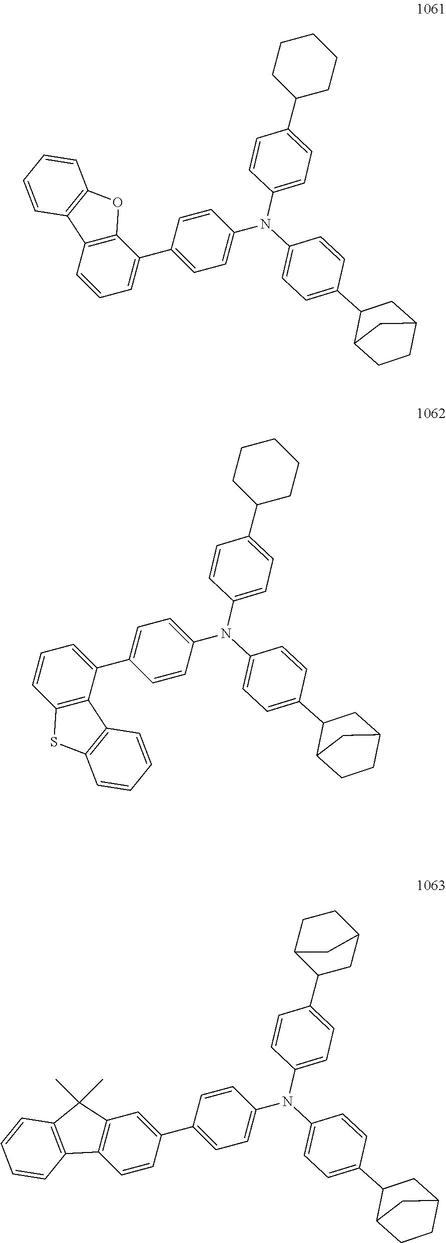 [SMILES: c1ccc2c(c1)oc1c(-c3ccc(N(c4ccc(C5CCCCC5)cc4)c4ccc(C5CC6CCC5C6)cc4)cc3)cccc12 ; c1ccc2c(c1)sc1cccc(-c3ccc(N(c4ccc(C5CCCCC5)cc4)c4ccc(C5CC6CCC5C6)cc4)cc3)c12 ; CC1(C)c2ccccc2-c2ccc(-c3ccc(N(c4ccc(C5CC6CCC5C6)cc4)c4ccc(C5CC6CCC5C6)cc4)cc3)cc21] 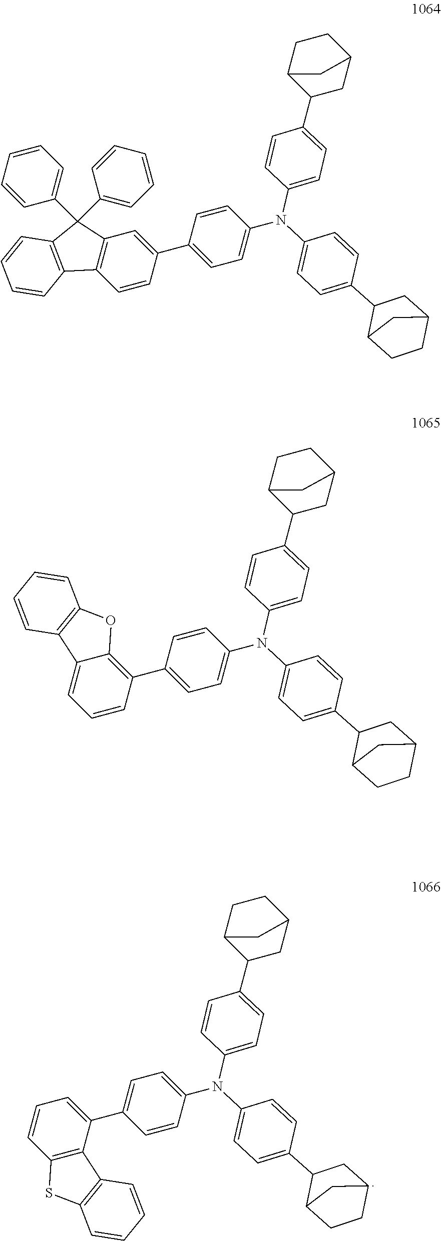 [SMILES: c1ccc(C2(c3ccccc3)c3ccccc3-c3ccc(-c4ccc(N(c5ccc(C6CC7CCC6C7)cc5)c5ccc(C6CC7CCC6C7)cc5)cc4)cc32)cc1 ; c1ccc2c(c1)oc1c(-c3ccc(N(c4ccc(C5CC6CCC5C6)cc4)c4ccc(C5CC6CCC5C6)cc4)cc3)cccc12 ; c1ccc2c(c1)sc1cccc(-c3ccc(N(c4ccc(C5CC6CCC5C6)cc4)c4ccc(C5CC6CCC5C6)cc4)cc3)c12]
